(19) United States
(12) Patent Application Publication
(10) Pub. No.: US 2026/0184707 A1
(43) Pub. Date: Jul. 2, 2026

(54) PHARMACEUTICAL COMPOUNDS FOR THE TREATMENT OF COMPLEMENT MEDIATED DISORDERS

(71) Applicant: ALEXION PHARMACEUTICALS, INC., Boston, MA (US)

(72) Inventors: Venkat Rao GADHACHANDA, Hamden, CT (US); Satishkumar GADHIYA, Billerica, MA (US); Evans ONYANGO, Hamden, CT (US); Russell Glenn FRITZEMEIER, Hamden, CT (US); Darren Whitehouse, Westbrook, CT (US); Qi Zhou, Orange, CT (US)

(21) Appl. No.: 19/101,614

(22) PCT Filed: Aug. 8, 2023
(86) PCT No.: PCT/US2023/029711

§ 371 Date: Feb. 6, 2025

Related U.S. Application Data
(60) Provisional application No. 63/397,382, filed on Aug. 11, 2022.

(51) Int. Cl. C07D 471/04; A61K 31/437; A61K 31/444; A61K 31/4545; A61K 31/497; A61K 31/4985; A61K 31/506; A61K 31/513; A61K 31/53; A61K 31/5377; A61K 31/5386; A61K 31/541; A61K 31/55; A61K 31/675; C07D 405/14; C07D 409/12; C07D 409/14; C07D 417/14; C07D 487/04; C07D 495/04; C07D 513/04; C07D 519/00; C07F 9/6561

(52) U.S. Cl. CPC C07D 471/04; A61K 31/437; A61K 31/444; A61K 31/4545; A61K 31/497; A61K 31/4985; A61K 31/506; A61K 31/513; A61K 31/53; A61K 31/5377; A61K 31/5386; A61K 31/541; A61K 31/55; A61K 31/675; C07D 405/14; C07D 409/12; C07D 409/14; C07D 417/14; C07D 487/04; C07D 495/04; C07D 513/04; C07D 519/00; C07F 9/6561

(57) ABSTRACT

This disclosure provides compounds, compositions, and methods to treat medical disorders, such as complement-mediated disorders, including complement C1s-mediated disorders.

### FIELD OF THE DISCLOSURE

Herein are provided pharmaceutical compounds to treat medical disorders, such as complement-mediated disorders, including complement C1-mediated disorders.

### BACKGROUND OF THE DISCLOSURE

The complement system is a part of the innate immune system which does not adapt to changes over the course of the subject's life but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells), and agglutination (clustering and binding of pathogens together).

The complement system has three pathways: classical, alternative, and lectin. The classical pathway is triggered by antibody-antigen complexes with the antibody isotypes IgG and IgM. The antibody-antigen complex binds to C1 and this initiates the cleavage of C4 and C2 to generate C3 convertase that then splits C3 into C3a and C3b. C3a interacts with its C3a receptor to recruit leukocytes, while C3b binds to C3 convertase to form C5 convertase. C5 convertase cleaves C5 into C5a and C5b. Similar to C3a, C5a interacts with its C5a receptor to recruit leukocytes, but C5b interacts with C6, C7, C8, and C8 and together these proteins form the cylindrical membrane attack complex (MAC) that causes the cell to swell and burst. These immune responses can be inhibited by preventing C1 from being able to bind the antibody-antigen complex.

Given the range of serious diseases mediated by a disfunction of the complement system, there is a clear medical need to provide pharmaceutically acceptable compounds, methods, compositions, and methods of manufacture to inhibit the complement system in a patient in need thereof.

Therefore, the present disclosure provides compounds and their uses and compositions to treat disorders arising from or amplified by a disfunction of the complement system. The present disclosure also provides compounds, uses, compositions, combinations, and processes of manufacture that inhibit C1s (complement C1 esterase) and thus can treat disorders mediated by C1s.

### SUMMARY

The present disclosure provides compounds, compositions, and methods for treating a disorder mediated by the complement cascade (including a dysfunctional cascade), a disorder or abnormality of a cell that adversely affects the ability of the cell to engage in or respond to normal complement activity including for example, the classical complement pathway, or an undesired complement-mediated response to a medical treatment, such as surgery or other medical procedure or a pharmaceutical or biopharmaceutical drug administration, a blood transfusion, or other allogenic tissue or fluid administration.

In some embodiments, the active compound may act as an inhibitor of the complement classical pathway by inhibiting complement C1s.

Without wishing to be bound by theory, the present disclosure is based, in part, on the unexpected discovery that compounds of the disclosure exhibit advantageous properties over other C1s inhibitors (e.g., the compounds described in WO 2020/198062 and WO2022/066774), such as improved C1s inhibiting activity, improved classical pathway hemolysis inhibiting activity, improved Caco-2 permeability, improved oral bioavailability, improved C1s selectivity (e.g., over other proteases, such as MASP-2), and/or improved metabolic stability. The present disclosure is also based, in part, on the unexpected discovery that compounds of the disclosure exhibit improved selectivity for C1s over other proteases (e.g., MASP-2), e.g., as compared to the compounds described in WO 2019/231935.

In one aspect, the present disclosure provides a compound of Formula (I):

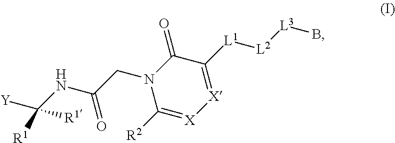

or a pharmaceutically acceptable salt thereof, in which all variables are as defined herein.

In another aspect, the present disclosure provides a pharmaceutical composition including a compound disclosed herein (e.g., any one of the compounds of formula (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof and a pharmaceutically acceptable excipient.

In another aspect, the present disclosure provides a method of treating a complement C1 esterase (C1s) mediated disorder. The method includes administering to a subject in need thereof a therapeutically effective amount of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof.

In another aspect, the present disclosure provides a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof, for use in the treatment of a C1s mediated disorder.

In another aspect, the present disclosure provides a use of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof in the preparation a medicament for use in the treatment of a C1s mediated disorder.

### Definitions

Compounds are described using standard nomenclature. Unless defined otherwise, all technical and scientific terms used herein have the same meaning as is commonly understood by one of skill in the art to which the present disclosure belongs.

The terms “a” and “an” do not denote a limitation of quantity, but rather denote the presence of at least one of the referenced items. The term “or” means “and/or.” Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely as illustration, and does not pose a limitation on the scope of the invention.

The term “alkoxy,” as used herein, refers to a —OR radical, in which R is alkyl, as defined herein.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-8, 1-6, 1-4, or 1-2 carbon atoms (e.g., C₁-C₈, C₁-C₆, C₁-C₄, or C₁-C₂). Examples include, but are not limited to, methyl, ethyl, isobutyl, sec-butyl, tert-butyl, 2-methylpropyl, and 2,2-dimethylpropyl.

The term “alkylene,” as used herein, refers to a divalent radical obtained by removing a hydrogen atom from a carbon atom of an alkyl group. The divalency of an alkylene group does not include the optional substituents on the alkylene group. Examples of alkylene groups include, but are not limited to, methylene, ethylene, and n-propylene.

The term “amino,” as used herein, refers to a monovalent radical of formula —NH₂. An “optionally substituted amino,” as used herein, refers to an amino group in which one or both hydrogen atoms are independently replaced with a substituent as defined herein.

The term “aryl,” as used herein, refers to any monocyclic or fused ring bicyclic or multicyclic system containing only carbon atoms in the ring(s), which has the characteristics of aromaticity in terms of electron distribution throughout the entire ring system, e.g., phenyl, naphthyl, or phenanthryl. An aryl group may have, e.g., 6-16, 6-14, or 6-10 carbon ring atoms (e.g., C₆-C₁₆, C₆-C₁₄, C₆-C₁₀, C₆, C₁₀, C₁₄, or C₁₆).

The term “arylene,” as used herein refers to a divalent radical obtained by removing a hydrogen atom from a carbon atom of an aryl group. The divalency of an arylene group does not include the optional substituents on the arylene group. Phenylene is a non-limiting example of an arylene group.

The term “aryloxy,” as used herein, refers to an —OR radical, in which R is aryl, as defined herein.

The term “carbocyclyl,” as used herein, refers to a monovalent, saturated (i.e., cycloalkyl) or unsaturated, non-aromatic group (e.g., cycloalkenyl, which contains at least one carbon-carbon double bond and no carbon-carbon triple bonds) containing only C and H when unsubstituted, which may be monocyclic, bicyclic, or multicyclic (e.g., tricyclic). A carbocyclyl may have, e.g., 3-14 carbons (e.g., a C₃-C₄, C₃-C₅, C₃-C₆, C₃-C₇, C₃-C₈, or C₃-C₁₄ carbocyclyl). Examples of carbocyclyls include, but are not limited to, cyclopropyl, cyclobutyl, cyclopentyl, cyclohexyl, cycloheptyl, cyclohexenyl, cycloheptenyl, and fluorenyl. The term “carbocyclyl” also includes cyclic groups having a bridged multicyclic structure in which one or more carbons bridges two non-adjacent members of a monocyclic ring, e.g., bicyclo[2.2.1]heptyl.

The term “halo,” as used herein, refers to a fluorine (fluoro; F), chlorine (chloro; Cl), bromine (bromo; Br), or iodine (iodo; I) radical.

The term “heteroaryl,” as used herein, refers to a monocyclic, bicyclic, or multicyclic aromatic ring monocyclic, bicyclic, or multicyclic group containing 1, 2, 3, or 4 heteroatoms selected from N, O, S, B, and P (e.g., 1-4, 1-3, or 1 or 2 heteroatoms selected from N, O, and S) as ring atoms, with the remaining ring atoms being carbon. In some embodiments, a heteroaryl group is a bicyclic or tricyclic system containing at least one 5, 6, or 7 membered aromatic ring which contains from 1, 2, 3, or 4 heteroatoms selected from N, O, S, B or P (e.g., 1-4, 1-3, or 1 or 2 heteroatoms selected from N, O, and S) as ring atoms, with the remaining ring atoms being carbon. In some embodiments, a heteroaryl group is a monocyclic aromatic ring having 5 or 6 ring atoms (i.e., 5- or 6-membered heteroaryl). In some embodiments, is a bicyclic aromatic ring system having 8 to 10 ring atoms (i.e., 8- to 10-membered bicyclic heteroaryl). Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl.

The term “heteroaryloxy,” as used herein, refers to a monovalent radical of formula —OR, in which R is heteroaryl, as defined herein.

The term “heterocyclyl,” as used herein, refers to saturated or unsaturated, non-aromatic, monocyclic, bicyclic, or multicyclic group containing 1, 2, 3, or 4 heteroatoms selected from N, O, S, B, and P (e.g., 1-4, 1-3, or 1 or 2 heteroatoms selected from N, O, and S) as ring atoms, with the remaining ring atoms being carbon. The term “heterocyclyl” includes, e.g., monocyclic 3- to 12-membered rings, bicyclic 5- to 16-membered ring systems, multicyclic (e.g., tricyclic) 10- to 18-membered ring systems, which may include bridged ring systems when bicyclic or multicyclic. In some embodiments, a heterocyclyl group contains 3-16 ring atoms (i.e., 3- to 16-membered heterocyclyl), e.g., 3-12 ring atoms (i.e., 3- to 12-membered heterocyclyl) or 4-10 ring atoms (i.e., 4- to 10-membered heterocyclyl). Examples of saturated heterocyclyl groups include saturated 4- to 7-membered monocyclic groups containing 1 to 4 nitrogen atoms (e.g., pyrrolidinyl, imidazolidinyl, piperidinyl, pyrrolinyl, azetidinyl, piperazinyl, and pyrazolidinyl); saturated 4 to 6-membered monocyclic groups containing 1 to 2 oxygen atoms and 1 to 3 nitrogen atoms (e.g., morpholinyl); saturated 3 to 6-membered monocyclic groups containing 1 to 2 sulfur atoms and 1 to 3 nitrogen atoms (e.g., thiazolidinyl). Examples of unsaturated, non-aromatic heterocyclyl radicals include but are not limited to, dihydrothienyl, dihydropyranyl, dihydrofuryl, and dihydrothiazolyl. Other examples of heterocyclyl radicals include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ-benzo[d]isothiazol-6-yl, dihydropyranyl, dihydrofuryl and dihydrothiazolyl. “Bicyclic heterocyclyl” includes groups in which a saturated or unsaturated, non-aromatic ring containing 1, 2, 3, or 4 heteroatoms as ring atoms is fused with an aryl group (e.g., phenyl) or a cycloalkyl group. “Bicyclic heterocyclyl” also includes groups in which a heteroaryl group, as defined herein, is fused to a saturated or unsaturated, non-aromatic ring containing 0, 1, 2, 3, or 4 heteroatoms as ring atoms.

The term “heterocyclyloxy,” as used herein, refers to a monovalent radical of formula —OR, in which R is heterocyclyl, as defined herein.

The term “oxo,” as used herein, refers to a ═O radial.

The term “substituted”, as used herein, means that any one or more hydrogens on the designated atom or group is replaced with a moiety as defined herein or selected from an indicated group of moieties, provided that the designated atom's normal valence is not exceeded, and the resulting compound is stable. For example, when the substituent is oxo (i.e., ═O), then two hydrogens on the atom are replaced. For example, a pyridyl group substituted by oxo is a pyridone. Combinations of substituents and/or variables are permissible only if such combinations result in stable compounds or useful synthetic intermediates. The phrase “optionally substituted X,” as used herein, is intended to be equivalent to “X, in which X is optionally substituted” (e.g., “alkyl, in which said alkyl is optionally substituted”). It is not intended to mean that the feature “X” (e.g., alkyl) per se is optional. The term “optionally substituted,” as used herein, refers to having 0, 1, or more substituents (e.g., 0-10 substituents, 0-5 substituents, or 0, 1, 2, 3, 4, 5, 6, 7, 8, 9, or 10 substituents).

Alkyl, alkylene, alkoxy, amino, carbocyclyl, aryl, arylene, aryloxy, heteroaryl, and heterocyclyl groups may be substituted with carbocyclyl (e.g., cycloalkyl); aryl; heteroaryl; heterocyclyl; halo; OR, in which R is H, alkyl, carbocyclyl (e.g., cycloalkyl), aryl, heteroaryl, or heterocyclyl; SR, in which R is H, alkyl, cycloalkyl, aryl, heteroaryl, or heterocyclyl; CN; NO₂; N₃; NRR′; in which each of R and R′ is, independently, H, alkyl, cycloalkyl, aryl, heteroaryl, or heterocyclyl; SO₂R, in which R is H, alkyl, or aryl; SO₂NRR′, in which each of R and R′ is, independently, H, alkyl, or aryl; SOR, in which R is H, alkyl, or aryl; or P(O)(OR)₂, in which each R is, independently, H or alkyl. Amino, aryl, carbocyclyl, heteroaryl, and heterocyclyl groups may also be substituted with alkyl. Alkyl, alkylene, carbocyclyl, and heterocyclyl groups may also be substituted with oxo or ═NR, in which R is H or alkyl. Alkyl and alkylene groups may also be substituted with spirocyclic carbocycle (e.g., spirocyclic cycloalkyl) or spirocyclic heterocyclyl. In some embodiments, a substituent is further substituted with one or more substituents as described herein. For example, a C₁ alkyl group, i.e., methyl, may be substituted with oxo to form a formyl group and further substituted with —OH or —NR₂ to form a carboxyl group or an amido group.

The term “complement-mediated disorder,” as used herein, refers to a disorder in which the amount or activity of complement is such as to cause disorder in an individual.

As used herein, a compound having “complement C1 esterase (C1s) inhibiting activity” refers to a compound exhibiting an IC₅₀ of less than 100 nM against as determined with a human complement C1s enzyme assay as described in Example 3 herein.

The term “pharmaceutical composition,” as used herein, refers to one or more active compounds, formulated together with one or more pharmaceutically acceptable excipients. In some embodiments, a compound of the disclosure (e.g., is present in a unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, or capsules; and parenteral administration, for example, by subcutaneous, intramuscular, or intravenous injection.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid.

The term “pharmaceutically acceptable excipient,” as used herein, refers to any inactive ingredient (for example, a vehicle capable of suspending or dissolving the active compound) that is biocompatible and suitable for administration to a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Those of ordinary skill in the art are familiar with a variety of agents and materials useful as excipients.

The term “subject,” as used herein, can be a human, non-human primate, or other non-human mammal, such as but not limited to dog, cat, horse, cow, pig, goat, monkey, rat, mouse, and sheep. In preferred embodiments, the subject is a human.

As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms or conditions; diminishment of extent of disease, disorder, or condition; stabilizing (i.e., not worsening) of the state of disease, disorder, or condition; delay or slowing in the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or the time course of the progression is slowed or lengthened, as compared to the extent or time course in the absence of treatment.

A “therapeutically effective amount” or an “effective amount” of an active compound pharmaceutical composition of the present disclosure refers an amount effective, when administered to a subject, to provide a therapeutic benefit, such as an amelioration of symptoms or reduction or diminution of the disease itself. In one embodiment, a therapeutically effective amount is an amount sufficient to prevent a significant increase, or will significantly reduce, the detectable level of hemolysis in the patient's blood, serum, or tissues.

### DETAILED DESCRIPTION

### Active Compounds

The present disclosure provides compounds and salts useful for the treatment of a disorder mediated by the complement cascade (e.g., a disorder mediated by C1s). In some embodiments, a compound of the present disclosure is described by Formula (I′):

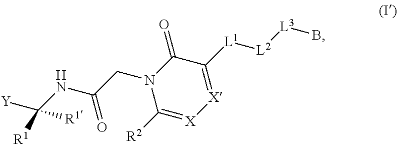

or a pharmaceutically acceptable salt thereof, in which
each of R¹ and R^{1′} is independently H or optionally substituted C₁-C₆ alkyl;
X is CR³ or N;
X is CR⁴ or N;
R² is H, C₁-C₆ alkyl, optionally substituted C₆-C₁₄ aryl, optionally substituted C₃-C₈ carbocyclyl, optionally substituted 5- to 10-membered heteroaryl, optionally substituted 4- to 10-membered heterocyclyl, optionally substituted (4- to 10-membered heterocyclyl)oxy, or optionally substituted (5- to 9-membered heteroaryl)oxy;
each of R³ and R⁴ is independently H, halo, or optionally substituted C₁-C₆ alkyl;
L¹ is a bond; NH; NHC(O); NHC(O)O; NHC(O)NH, or NHS(O)₂;
L² is a bond or optionally substituted C₁-C₆ alkylene;
L³ is a bond, NH, NHC(O), C(O), O, S(O)₂CH₂; B is halo, optionally substituted C₆-C₁₄ aryl; optionally substituted C₃-C₁₄ carbocyclyl; optionally substituted 5- to 14-membered heterocyclyl; or optionally substituted 5- to 10-membered heteroaryl; and
Y is

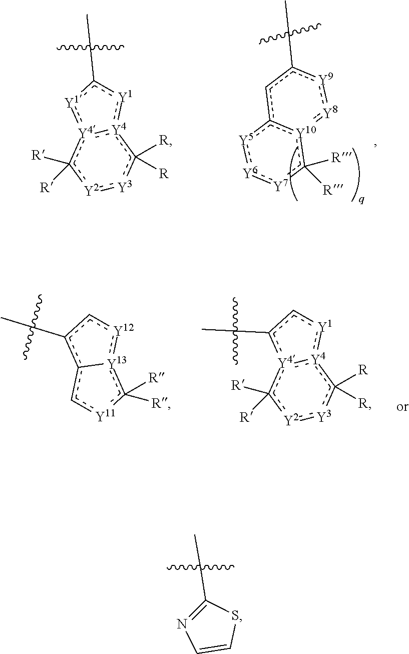

in which
Y¹ is O, S, NR^{d}, wherein each R^{d} is independently absent, H, or C₁-C₆ alkyl;
Y^{1′} is O, S, NR^{d}, or C(R^{d})₂;
each of Y² and Y³ is independently NR^{e} or C(R^{e})₂, wherein each R^{e} is independently absent; H; optionally substituted C₁-C₆ alkyl; halo; or N(R^{g})₂, wherein each R^{g} is independently H or C₁-C₆ alkyl; or both R^{e} combine to form oxo;
each of Y⁴, Y⁴, Y¹⁰, and Y¹³ is independently CR^{e} or N;
each of Y⁵, Y⁶, and Y⁷ is independently O, S, NR^{f} or C(R^{f})₂, wherein each R^{f} is independently absent; H; optionally substituted C₁-C₆ alkyl; halo; or N(R^{g})₂; or both R^{f} combine to form oxo;
each of Y⁸ and Y⁹ is independently C(R^{f})₂ or NR^{f};
each of Y¹¹ and Y¹² is independently NR^{e}, C(R^{e})₂, S, or O;
each [symbol] is independently a single bond or a double bond;
each of R, R′, R″, and R′″ is independently absent, H, optionally substituted C₁-C₆ alkyl, halo, or N(R^{g})₂; or
both R combine to form oxo; or
both R′ combine to form oxo; and
q is 0 or 1.

In some embodiments, a compound of the present disclosure is a compound of Formula (I):

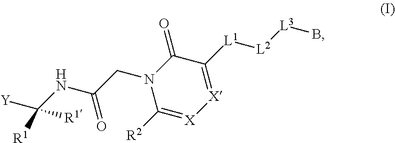

or a pharmaceutically acceptable salt thereof, in which all variables are as defined for Formula (I′), provided that at least one of the following is true:
(i) -L¹-L²-L³- do not combine to form

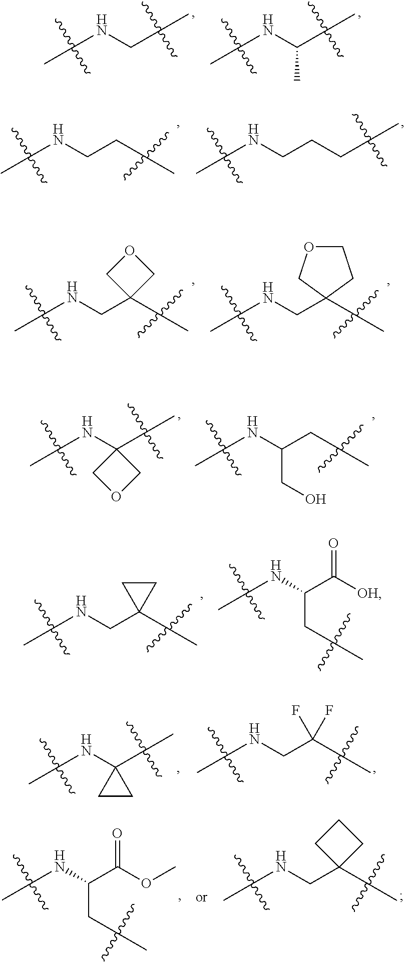

(ii) R² is not C₁-C₆ alkyl,

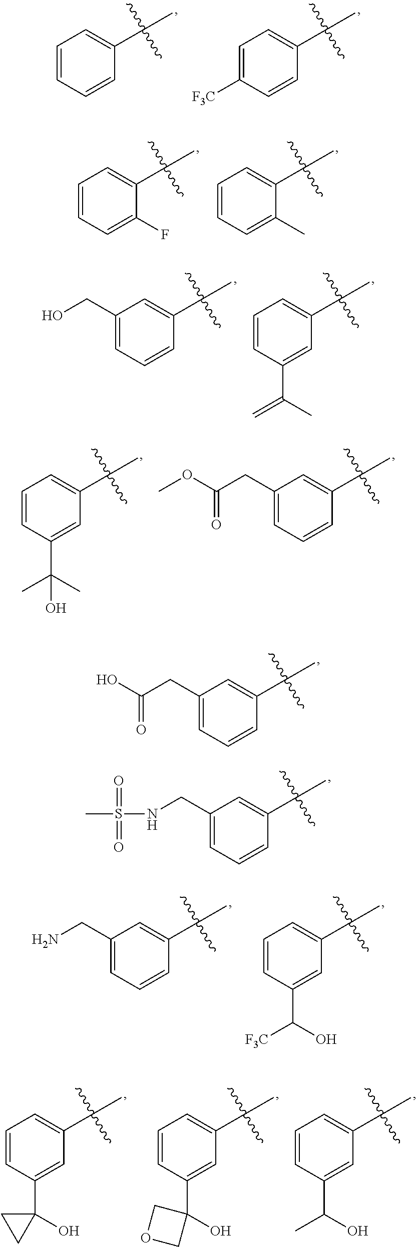

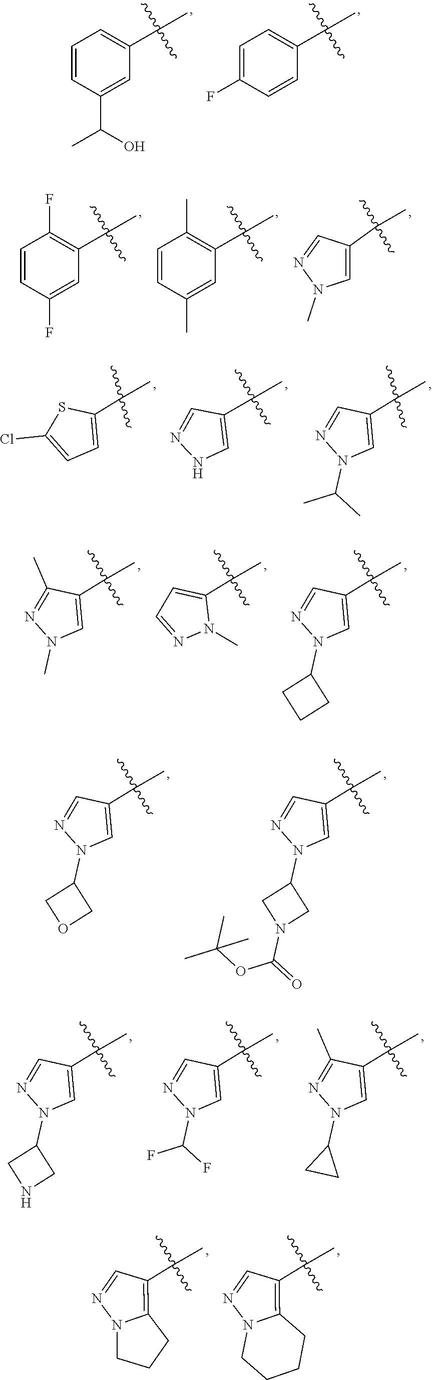

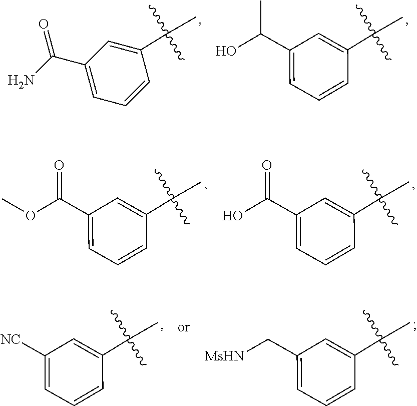

(iii) B is not

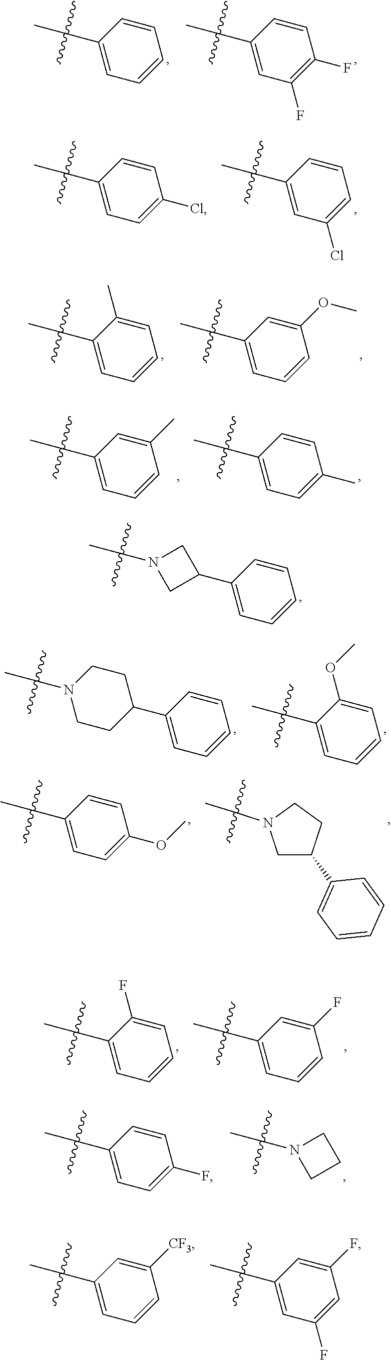

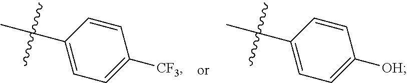 [SMILES: *c1ccc(C(F)(F)F)cc1 ; *c1ccc(O)cc1]

(iv) at least one of R¹ and R^{1′} is not H;
(v) X is N;
(vi) X′ is CR⁴, and
(vii) Y is not

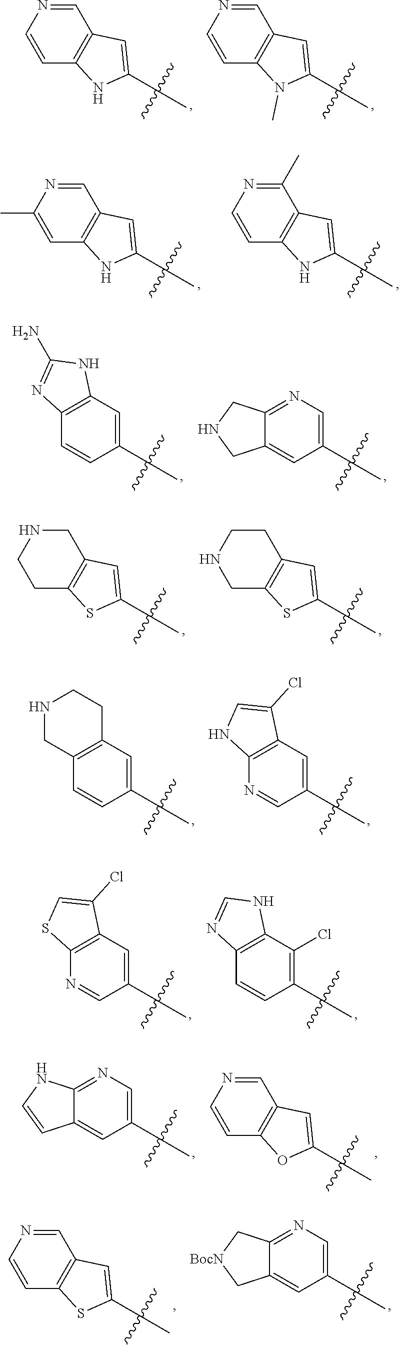

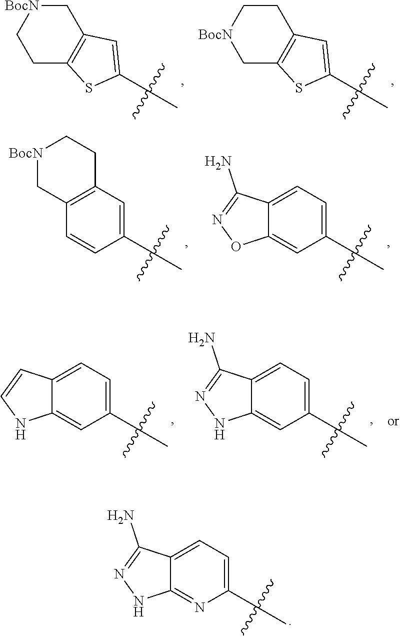

In some embodiments, R¹ is H. In some embodiments, R¹ is optionally substituted C₁-C₆ alkyl. In some embodiments, R¹ is methyl. In some embodiments, R¹ is CH₂OH.

In some embodiments, R¹ is H. In some embodiments, R¹ is optionally substituted C₁-C₆ alkyl. In some embodiments, R¹ is methyl. In some embodiments, R¹ is CH₂OH.

In some embodiments, X is CR³. In some embodiments, X is CH. In some embodiments, X is CCH₃. In some embodiments, X is N.

In some embodiments, X is CR⁴. In some embodiments, X′ is CH. In some embodiments, X′ is CCH₃. In some embodiments, X′ is N.

In some embodiments, R² is optionally substituted C₆-C₁₄ aryl, optionally substituted 5- to 10-membered heteroaryl, optionally substituted 4- to 10-membered heterocyclyl, or optionally substituted (4- to 10-membered heterocyclyl)oxy, or optionally substituted (5- to 9-membered heteroaryl)oxy.

In some embodiments, R² is optionally substituted phenyl, e.g., phenyl optionally substituted with one or more substituents independently selected from halo; optionally substituted C₁-C₆ alkyl; optionally substituted C₁-C₆ alkoxy; optionally substituted (4- to 10-membered heterocyclyl)oxy; P(O)(OH)CH₃; P(O)(OR′″)₂, wherein each R′″ is independently H or C₁-C₆ alkyl; S(O)₂CH₃; optionally substituted 4- to 10-membered heterocyclyl; SFs; S(O)(NCN)CH₃, S(O)(NH)CH₃; and optionally substituted amino. In some embodiments, R² is

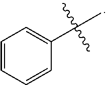 [SMILES: *c1ccccc1]

In some embodiments, R² is

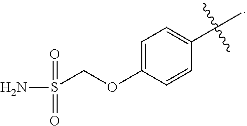 [SMILES: *c1ccc(OCS(N)(=O)=O)cc1]

In some embodiments, R² is

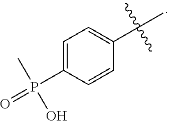 [SMILES: *c1ccc(P(C)(=O)O)cc1]

In some embodiments, R² is

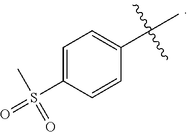 [SMILES: *c1ccc(S(C)(=O)=O)cc1]

In some embodiments, R² is

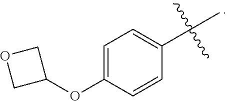 [SMILES: *c1ccc(OC2COC2)cc1]

In some embodiments, R² is

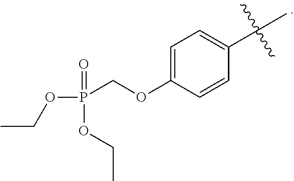 [SMILES: *c1ccc(OCP(=O)(OCC)OCC)cc1]

In some embodiments, R² is

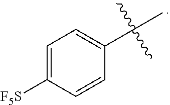

In some embodiments, R² is

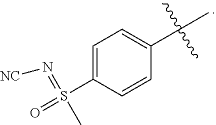 [SMILES: *c1ccc(S(C)(=O)=NC#N)cc1]

In some embodiments, R² is

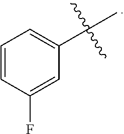 [SMILES: *c1cccc(F)c1]

In some embodiments, R² is

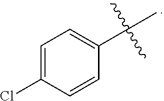 [SMILES: *c1ccc(Cl)cc1]

In some embodiments, R² is

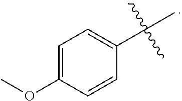 [SMILES: *c1ccc(OC)cc1]

In some embodiments, R² is

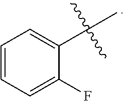 [SMILES: *c1ccccc1F]

In some embodiments, R² is

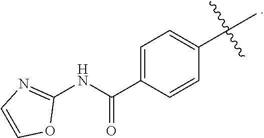 [SMILES: *c1ccc(C(=O)Nc2ncco2)cc1]

In some embodiments, R² is

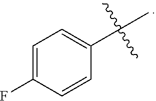 [SMILES: *c1ccc(F)cc1]

In some embodiments, R² is

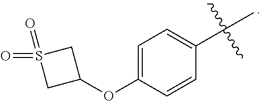 [SMILES: *c1ccc(OC2CS(=O)(=O)C2)cc1]

In some embodiments, R² is

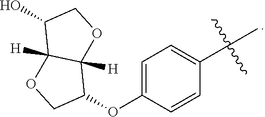

In some embodiments R² is

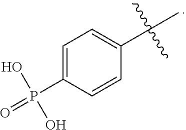 [SMILES: *c1ccc(P(=O)(O)O)cc1]

In some embodiments, R² is

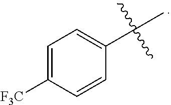 [SMILES: *c1ccc(C(F)(F)F)cc1]

In some embodiments, R² is

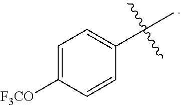 [SMILES: *c1ccc(OC(F)(F)F)cc1]

In some embodiments, R² is

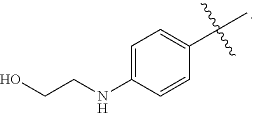 [SMILES: *c1ccc(NCCO)cc1]

In some embodiments, R² is

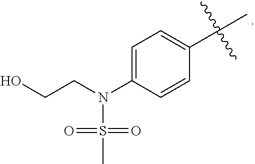 [SMILES: *c1ccc(N(CCO)S(C)(=O)=O)cc1]

In some embodiments, R² is

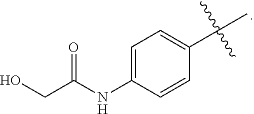 [SMILES: *c1ccc(NC(=O)CO)cc1]

In some embodiments, R² is

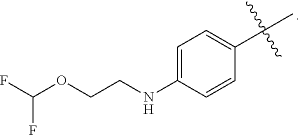 [SMILES: *c1ccc(NCCOC(F)F)cc1]

In some embodiments, R² is

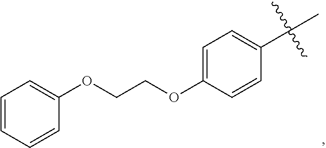 [SMILES: *c1ccc(OCCOc2ccccc2)cc1]

In some embodiments, R² is

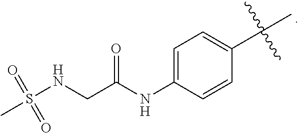 [SMILES: *c1ccc(NC(=O)CNS(C)(=O)=O)cc1]

In some embodiments, R² is

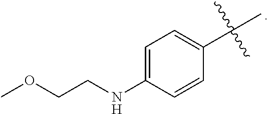 [SMILES: *c1ccc(NCCOC)cc1]

In some embodiments, R² is

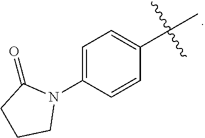 [SMILES: *c1ccc(N2CCCC2=O)cc1]

In some embodiments, R² is

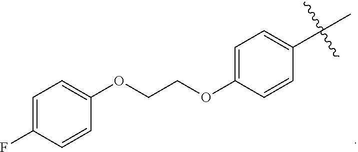 [SMILES: *c1ccc(OCCOc2ccc(F)cc2)cc1]

In some embodiments, R² is

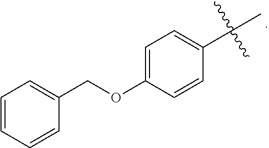 [SMILES: *c1ccc(OCc2ccccc2)cc1]

In some embodiments, R² is

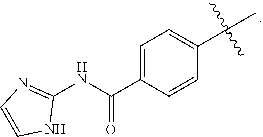 [SMILES: *c1ccc(C(=O)Nc2ncc[nH]2)cc1]

In some embodiments, R² is

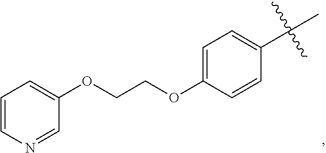 [SMILES: *c1ccc(OCCOc2cccnc2)cc1]

In some embodiments, R² is

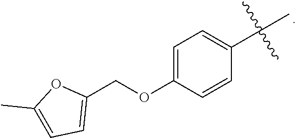 [SMILES: *c1ccc(OCc2ccc(C)o2)cc1]

In some embodiments, R² is

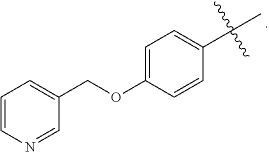 [SMILES: *c1ccc(OCc2cccnc2)cc1]

In some embodiments, R² is

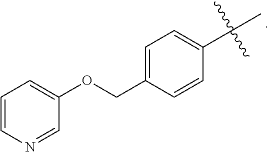 [SMILES: *c1ccc(COc2cccnc2)cc1]

In some embodiments, R² is

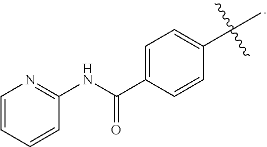 [SMILES: *c1ccc(C(=O)Nc2ccccn2)cc1]

In some embodiments, R² is

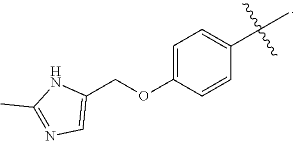 [SMILES: *c1ccc(OCc2cnc(C)[nH]2)cc1]

In some embodiments, R² is

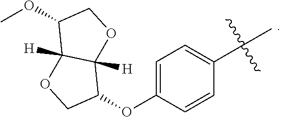

In some embodiments, R² is

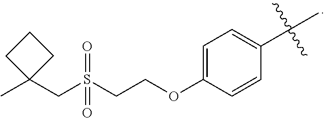 [SMILES: *c1ccc(OCCS(=O)(=O)CC2(C)CCC2)cc1]

In some embodiments, R² is

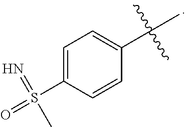 [SMILES: *c1ccc(S(C)(=N)=O)cc1]

In some embodiments, R² is

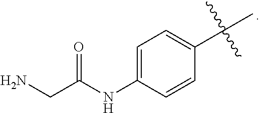 [SMILES: *c1ccc(NC(=O)CN)cc1]

In some embodiments, R² is

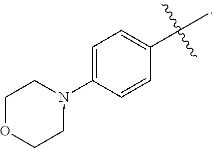 [SMILES: *c1ccc(N2CCOCC2)cc1]

In some embodiments, R² is

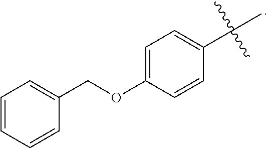 [SMILES: *c1ccc(OCc2ccccc2)cc1]

In some embodiments, R² is

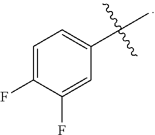 [SMILES: *c1ccc(F)c(F)c1]

In some embodiments, R² is

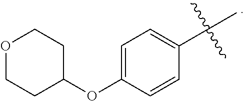 [SMILES: *c1ccc(OC2CCOCC2)cc1]

In some embodiments, R² is

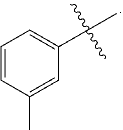 [SMILES: *c1cccc(C)c1]

In some embodiments, R² is

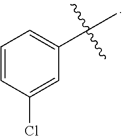 [SMILES: *c1cccc(Cl)c1]

In some embodiments, R² is

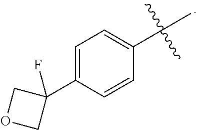 [SMILES: *c1ccc(C2(F)COC2)cc1]

In some embodiments, R² is

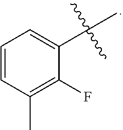 [SMILES: *c1cccc(C)c1F]

In some embodiments, R² is

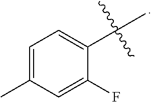 [SMILES: *c1ccc(C)cc1F]

In some embodiments, R² is

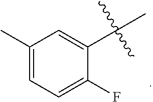 [SMILES: *c1cc(C)ccc1F]

In some embodiments, R² is

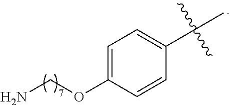

In some embodiments, R² is

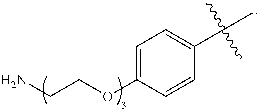

In some embodiments, R² is optionally substituted (4- to 10-membered heterocyclyl)oxy. In some embodiments, R² is

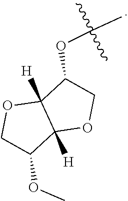 [SMILES: *O[C@@H]1CO[C@H]2[C@@H]1OC[C@H]2OC]

In some embodiments, R² is optionally substituted 4- to 10-membered heterocyclyl. In some embodiments, R² is

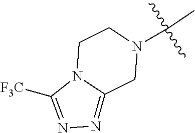 [SMILES: *N1CCn2c(nnc2C(F)(F)F)C1]

In some embodiments, R² is

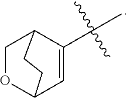 [SMILES: *C1=CC2CCC1CO2]

In some embodiments, R² is

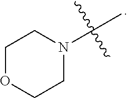 [SMILES: *N1CCOCC1]

In some embodiments, R² is

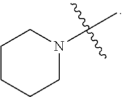 [SMILES: *N1CCCCC1]

In some embodiments, R² is

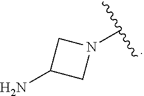 [SMILES: *N1CC(N)C1]

In some embodiments, R² is

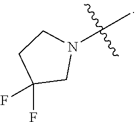 [SMILES: *N1CCC(F)(F)C1]

In some embodiments, R² is

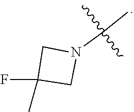 [SMILES: *N1CC(C)(F)C1]

In some embodiments, R² is

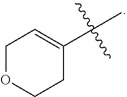 [SMILES: *C1=CCOCC1]

In some embodiments, R² is

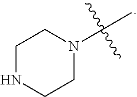 [SMILES: *N1CCNCC1]

In some embodiments, R² is

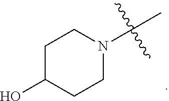 [SMILES: *N1CCC(O)CC1]

In some embodiments, R² is

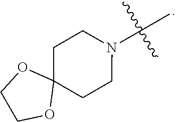 [SMILES: *N1CCC2(CC1)OCCO2]

In some embodiments, R² is

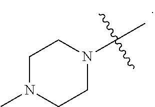 [SMILES: *N1CCN(C)CC1]

In some embodiments, R² is

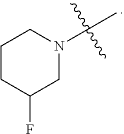 [SMILES: *N1CCCC(F)C1]

In some embodiments, R² is

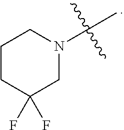 [SMILES: *N1CCCC(F)(F)C1]

In some embodiments, R² is

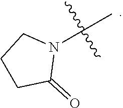 [SMILES: *N1CCCC1=O]

In some embodiments, R² is

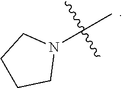 [SMILES: *N1CCCC1]

In some embodiments, R² is

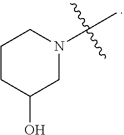 [SMILES: *N1CCCC(O)C1]

In some embodiments, R² is

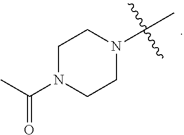 [SMILES: *N1CCN(C(C)=O)CC1]

In some embodiments, R² is

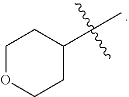 [SMILES: *C1CCOCC1]

In some embodiments, R² is

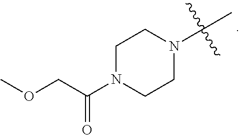 [SMILES: *N1CCN(C(=O)COC)CC1]

In some embodiments, R² is optionally substituted 5- to 10-membered heteroaryl. In some embodiments, R² is

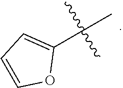 [SMILES: *c1ccco1]

In some embodiments, R² is

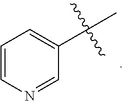 [SMILES: *c1cccnc1]

In some embodiments, R² is

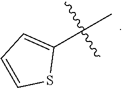 [SMILES: *c1cccs1]

In some embodiments, R² is

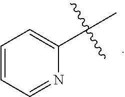 [SMILES: *c1ccccn1]

In some embodiments, R² is

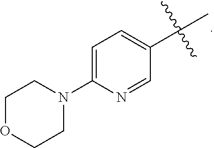 [SMILES: *c1ccc(N2CCOCC2)nc1]

In some embodiments, R² is

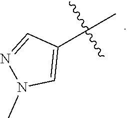 [SMILES: *c1cnn(C)c1]

In some embodiments, R² is

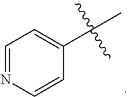 [SMILES: *c1ccncc1]

In some embodiments, R² is

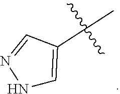 [SMILES: *c1cn[nH]c1]

In some embodiments, R² is

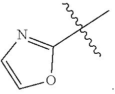 [SMILES: *c1ncco1]

In some embodiments, R² is

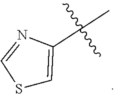 [SMILES: *c1cscn1]

In some embodiments, R² is

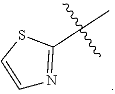 [SMILES: *c1nccs1]

In some embodiments, R² is

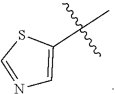 [SMILES: *c1cncs1]

In some embodiments, R² is

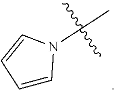 [SMILES: *n1cccc1]

In some embodiments, R² is optionally substituted C₃-C₈ carbocyclyl. In some embodiments, R² is optionally substituted C₃-C₈ cycloalkyl. In some embodiments, R² is

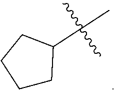 [SMILES: *C1CCCC1]

In some embodiments, R² is

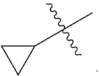 [SMILES: *C1CC1]

In some embodiments, R² is optionally substituted C₃-C₈ cycloalkenyl. In some embodiments, R² is

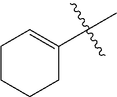 [SMILES: *C1=CCCCC1]

In some embodiments, R² is H. In some embodiments, R² is C₁-C₆ alkyl. In some embodiments, R² is CH₃. In some embodiments, R² is CH(CH₃)₂.

In some embodiments, L¹ is a bond. In some embodiments, L¹ is NH. In some embodiments, L¹ is NHS(O)₂. In some embodiments, L¹ is NHC(O). In some embodiments, L¹ is NHC(O)O. In some embodiments, L¹ is NHC(O)NH.

In some embodiments, L² is a bond. In some embodiments, L² is optionally substituted C₁-C₆ alkylene. In some embodiments, L² is C₁-C₆ alkylene. In some embodiments, L² is —CH₂—. In some embodiments, L² is —(CH₂)₂—. In some embodiments, L² is —(CH₂)₃—. In some embodiments, L² is —(CH₂)₄—. In some embodiments, L² is —(CH₂)₅—. In some embodiments, L² is

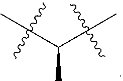

In some embodiments, L² is

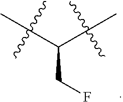

In some embodiments, L² is

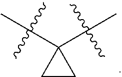 [SMILES: *C1(*)CC1]

In some embodiments, L² is

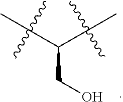

In some embodiments, L² is

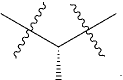

In some embodiments, L³ is a bond. In some embodiments, L³ is NH. In some embodiments, L³ is NHC(O). In some embodiments, L³ is C(O). In some embodiments, L³ is O. In some embodiments, L³ is SO₂CH₂.

In some embodiments, B is halo, e.g., Br.

In some embodiments, B is optionally substituted C₆-C₁₄ aryl or optionally substituted 5- to 10-membered heteroaryl.

In some embodiments, the compound is a compound of Formula (II):

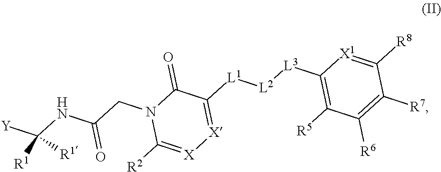

or a pharmaceutically acceptable salt thereof, in which X¹ is CR⁹ or N; each of R⁵, R⁶, and R⁹ is independently selected from H, halo, CN, SFs, optionally substituted C₁-C₆ alkyl, optionally substituted C₁-C6 alkoxy, S(O)(NH)CH₃, S(O)₂CH₃, and S(O)(NCN)CH₃; each of R⁷ and R⁸ is independently H, halo, CN, SFs, optionally substituted C₁-C₆ alkyl, optionally substituted C₁-C₆ alkoxy, optionally substituted amino, S(O)(NH)CH₃, S(O)₂CH₃, S(O)(NCN)CH₃, optionally substituted C₃-C₈ cycloalkyl, optionally substituted C₆-C₁₄ aryloxy, optionally substituted C₆-C₁₄ aryl, optionally substituted 5- to 10-membered heteroaryl, optionally substituted (5- to 10-membered heteroaryl)oxy, or optionally substituted (4- to 10-membered heterocyclyl)oxy, provided that no more than one of R⁷ and R⁸ is optionally substituted C₆-C₁₄ aryloxy, optionally substituted C₆-C₁₄ aryl, optionally substituted 5- to 10-membered heteroaryl, optionally substituted (5- to 10-membered heteroaryl)oxy, or optionally substituted (4- to 10-membered heterocyclyl)oxy; or R⁷ and R⁸, together with the atoms to which each is attached, form optionally substituted 5- to 6-membered heterocyclyl, optionally substituted 5- to 10-membered heteroaryl, or optionally substituted C₆-C₁₄ aryl; or R⁵ and A combine to for optionally substituted C₁-C₂ alkylene; R⁶ and R⁹ combine to form (C₂-C₆alkylene)(C₆-C₁₄arylene)(C₂-C₆alkylene), and each of R₅, R₇, and R₈ is H; and all other variables are as defined for Formula (I).

In some embodiments, the compound is a compound of Formula (IIA):

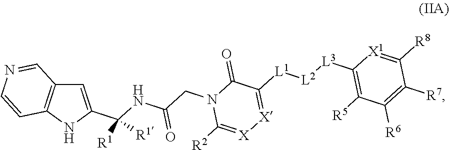

or a pharmaceutically acceptable salt thereof, in which all variables are as defined for Formula (II).

In some embodiments, X¹ is CR⁹. In some embodiments, X¹ is CH.

In some embodiments, R⁸ is H.

In some embodiments, R⁷ is optionally substituted C₆-C₁₄ aryl. In some embodiments, R⁷ is optionally substituted phenyl. In some embodiments, R⁷ is

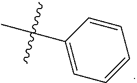 [SMILES: *c1ccccc1]

In some embodiments, R⁷ is

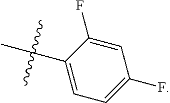 [SMILES: *c1ccc(F)cc1F]

In some embodiments, R⁷ is

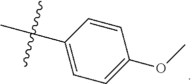 [SMILES: *c1ccc(OC)cc1]

In some embodiments, R⁷ is

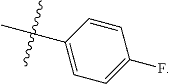 [SMILES: *c1ccc(F)cc1]

In some embodiments, R⁷ is

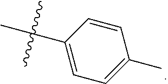 [SMILES: *c1ccc(C)cc1]

In some embodiments, R⁷ is

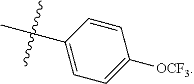 [SMILES: *c1ccc(OC(F)(F)F)cc1]

In some embodiments, R⁷ is

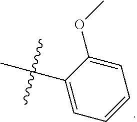 [SMILES: *c1ccccc1OC]

In some embodiments, R⁷ is

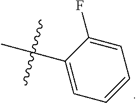 [SMILES: *c1ccccc1F]

In some embodiments, R⁷ is

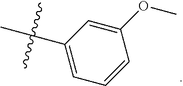 [SMILES: *c1cccc(OC)c1]

In some embodiments, R⁷ is optionally substituted 5- to 10-membered heteroaryl. In some embodiments, R⁷ is

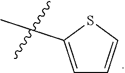 [SMILES: *c1cccs1]

In some embodiments, R⁷ is

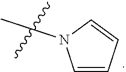 [SMILES: *n1cccc1]

In some embodiments, R⁷ is

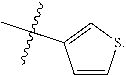 [SMILES: *c1ccsc1]

In some embodiments, R⁷ is

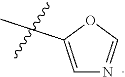 [SMILES: *c1cnco1]

In some embodiments, R⁷ is

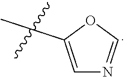 [SMILES: *c1cnco1]

In some embodiments, R⁷ is

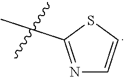 [SMILES: *c1nccs1]

In some embodiments, R⁷ is

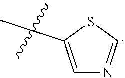 [SMILES: *c1cncs1]

In some embodiments, R⁷ is

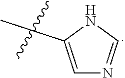 [SMILES: *c1cnc[nH]1]

In some embodiments, R⁷ is

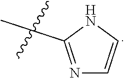 [SMILES: *c1ncc[nH]1]

In some embodiments, R⁷ is optionally substituted (5- to 10-membered heteroaryl)oxy. In some embodiments, R⁷ is

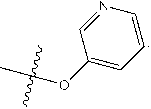 [SMILES: *Oc1cccnc1]

In some embodiments, R⁷ is

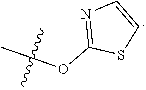 [SMILES: *Oc1nccs1]

In some embodiments, R⁷ is optionally substituted (4- to 10-membered heterocyclyl)oxy. In some embodiments, R⁷ is

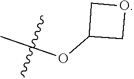 [SMILES: *OC1COC1]

In some embodiments, R⁷ is

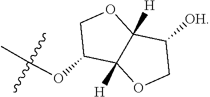 [SMILES: *O[C@@H]1CO[C@H]2[C@@H]1OC[C@H]2O]

In some embodiments, R⁷ is

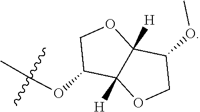 [SMILES: *O[C@@H]1CO[C@H]2[C@@H]1OC[C@H]2OC]

In some embodiments, R⁷ is

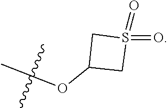 [SMILES: *OC1CS(=O)(=O)C1]

In some embodiments, R⁷ is optionally substituted C₃-C₈ cycloalkyl. In some embodiments, R⁷ is

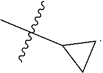 [SMILES: *C1CC1]

In some embodiments, R⁷ is optionally substituted 4- to 10-membered heterocyclyl. In some embodiments, R⁷ is

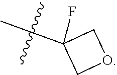 [SMILES: *C1(F)COC1]

In some embodiments, R⁷ is

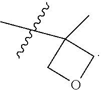 [SMILES: *C1(C)COC1]

In some embodiments, R⁸ is optionally substituted C₆-C₁₄ aryl, e.g., optionally substituted phenyl. In some embodiments, R⁸ is

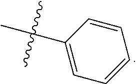 [SMILES: *c1ccccc1]

In some embodiments, R⁸ is

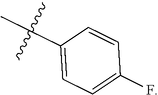 [SMILES: *c1ccc(F)cc1]

In some embodiments, R⁸ is optionally substituted 5- to 10-membered heteroaryl. In some embodiments, R⁸ is

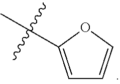 [SMILES: *c1ccco1]

In some embodiments, R⁸ is

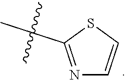 [SMILES: *c1nccs1]

In some embodiments, R⁸ is

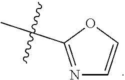 [SMILES: *c1ncco1]

In some embodiments, R⁷ is H.

In some embodiments, R⁵ is H. In some embodiments, R⁵ is optionally substituted C₁-C₆ alkoxy. In some embodiments, R⁵ is OCH₃. In some embodiments, R⁵ is halo. In some embodiments, R⁵ is F.

In some embodiments, R⁶ is H. In some embodiments, R⁶ is optionally substituted C₁-C₆ alkoxy. In some embodiments, R⁶ is OCH₃. In some embodiments, R⁶ is halo. In some embodiments, R⁶ is F.

In some embodiments, R⁶ and R⁹ combine to form (C₂-C₆alkylene)(C₆-C₄arylene)(C₂-C₆alkylene), and each of R₅, R₇, and R₈ is H. In some embodiments, B is

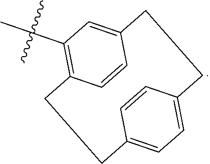 [SMILES: *c1cc2ccc1CCc1ccc(cc1)CC2]

In some embodiments, B is

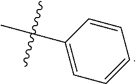 [SMILES: *c1ccccc1]

In some embodiments, B is

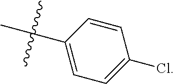 [SMILES: *c1ccc(Cl)cc1]

In some embodiments, B is

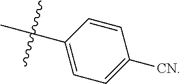 [SMILES: *c1ccc(C#N)cc1]

In some embodiments, B is

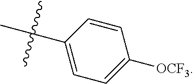 [SMILES: *c1ccc(OC(F)(F)F)cc1]

In some embodiments, B is

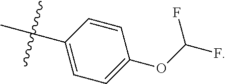 [SMILES: *c1ccc(OC(F)F)cc1]

In some embodiment, B is

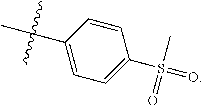 [SMILES: *c1ccc(S(C)(=O)=O)cc1]

In some embodiments, B is

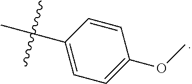 [SMILES: *c1ccc(OC)cc1]

In some embodiments, B is

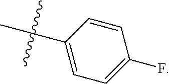 [SMILES: *c1ccc(F)cc1]

In some embodiments, B is

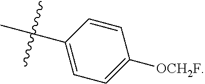

In some embodiments, B is

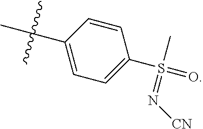 [SMILES: *c1ccc(S(C)(=O)=NC#N)cc1]

In some embodiments, B is

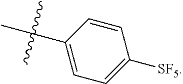

In some embodiments, B is

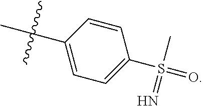 [SMILES: *c1ccc(S(C)(=N)=O)cc1]

In some embodiments, B is

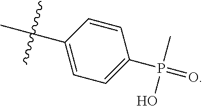 [SMILES: *c1ccc(P(C)(=O)O)cc1]

In some embodiments, B is

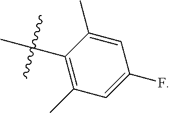 [SMILES: *c1c(C)cc(F)cc1C]

In some embodiments, the compound is a compound of Formula (III):

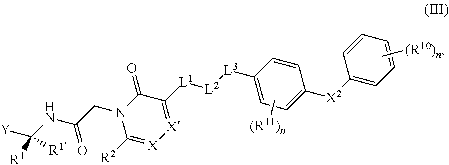

in which X² is O; C(R^{h})₂, wherein each R^{h} is independently hydrogen, halo, or optionally substituted C₁-C₆ alkyl, or both R^{h} combine to form oxo; S(O)₂, or NR^{h}; m is selected from 0, 1, 2, 3, 4, and 5; n is selected from 0, 1, 2, 3, and 4; and each R¹⁰ and R¹¹ is independently halo, CN, optionally substituted C₁-C₆ alkyl, optionally substituted C₁-C₆ alkoxy, or optionally substituted C₃-C₈ cycloalkyl, and all other variables are as defined for Formula (I).

In some embodiments, the compound is a compound of Formula (IIIA):

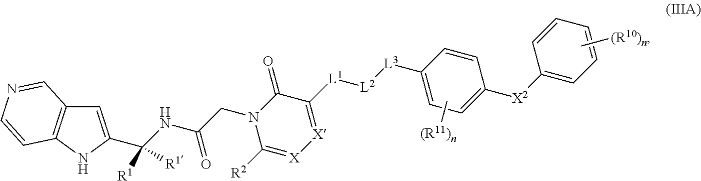

or a pharmaceutically acceptable salt thereof, in which all variables are as defined for Formula (III).

In some embodiments, the compound is a compound of Formula (IV):

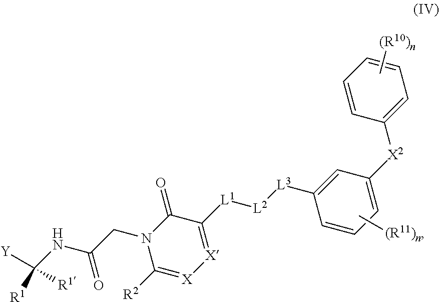

in which X² is O; C(R^{h})₂, wherein each R^{h} is independently hydrogen, halo, or optionally substituted C₁-C₆ alkyl, or both R^{h} combine to form oxo; S(O)₂, or NR^{h}; m is selected from 0, 1, 2, 3, 4, and 5; n is selected from 0, 1, 2, 3, and 4; and each R¹⁰ and R¹¹ is independently halo, CN, optionally substituted C₁-C₆ alkyl, optionally substituted C₁-C₆ alkoxy, or optionally substituted C₃-C₈ cycloalkyl, and all other variables are as defined for Formula (I).

In some embodiments, the compound is a compound of Formula (IVA):

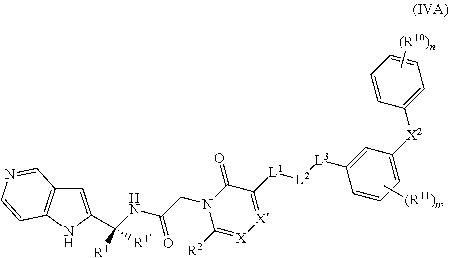

or a pharmaceutically acceptable salt thereof.

In some embodiments, X² is O. In some embodiments, X² is C(R^{a})₂. In some embodiments, X² is C(O). In some embodiments, X² is CF₂. In some embodiments, X² is S(O)₂.

In some embodiments, B is

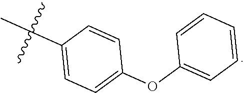 [SMILES: *c1ccc(Oc2ccccc2)cc1]

In some embodiments, B is

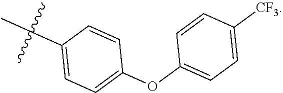 [SMILES: *c1ccc(Oc2ccc(C(F)(F)F)cc2)cc1]

In some embodiments, B is

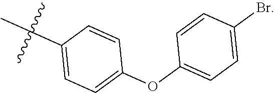 [SMILES: *c1ccc(Oc2ccc(Br)cc2)cc1]

In some embodiments, B is

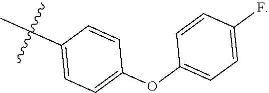 [SMILES: *c1ccc(Oc2ccc(F)cc2)cc1]

In some embodiments, B is

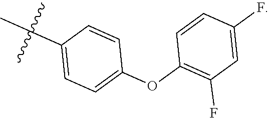 [SMILES: *c1ccc(Oc2ccc(F)cc2F)cc1]

In some embodiments, B is

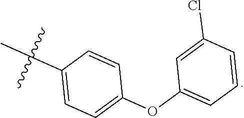 [SMILES: *c1ccc(Oc2cccc(Cl)c2)cc1]

In some embodiments, B is

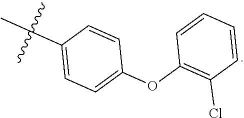 [SMILES: *c1ccc(Oc2ccccc2Cl)cc1]

In some embodiments, B is

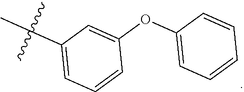 [SMILES: *c1cccc(Oc2ccccc2)c1]

In some embodiments, B is

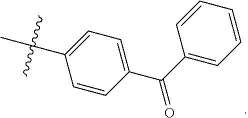 [SMILES: *c1ccc(C(=O)c2ccccc2)cc1]

In some embodiments, B is

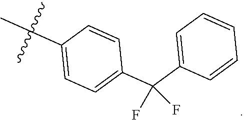 [SMILES: *c1ccc(C(F)(F)c2ccccc2)cc1]

In some embodiments, B is

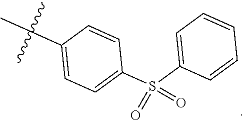 [SMILES: *c1ccc(S(=O)(=O)c2ccccc2)cc1]

In some embodiments, B is optionally substituted 5- to 10-membered heteroaryl. In some embodiments, B is

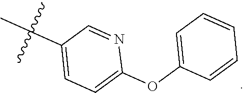 [SMILES: *c1ccc(Oc2ccccc2)nc1]

In some embodiments, B is

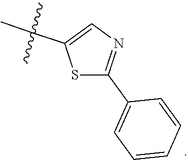 [SMILES: *c1cnc(-c2ccccc2)s1]

In some embodiments, B is

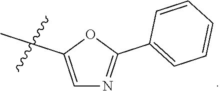 [SMILES: *c1cnc(-c2ccccc2)o1]

In some embodiments, B is

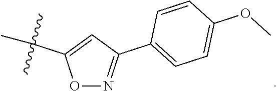 [SMILES: *c1cc(-c2ccc(OC)cc2)no1]

In some embodiments, B is

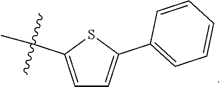 [SMILES: *c1ccc(-c2ccccc2)s1]

In some embodiments, B is

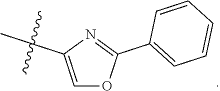 [SMILES: *c1coc(-c2ccccc2)n1]

In some embodiments, B is

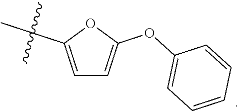 [SMILES: *c1ccc(Oc2ccccc2)o1]

In some embodiments, B is

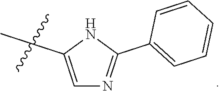 [SMILES: *c1cnc(-c2ccccc2)[nH]1]

In some embodiments, B is

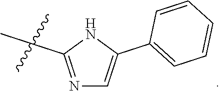 [SMILES: *c1ncc(-c2ccccc2)[nH]1]

In some embodiments, B is

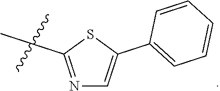 [SMILES: *c1ncc(-c2ccccc2)s1]

In some embodiments, B is

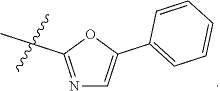 [SMILES: *c1ncc(-c2ccccc2)o1]

In some embodiments, B is

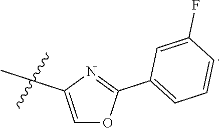 [SMILES: *c1coc(-c2cccc(F)c2)n1]

In some embodiments, B is

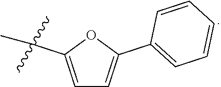 [SMILES: *c1ccc(-c2ccccc2)o1]

In some embodiments, B is

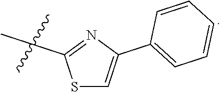 [SMILES: *c1nc(-c2ccccc2)cs1]

In some embodiments, B is

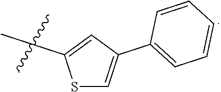 [SMILES: *c1cc(-c2ccccc2)cs1]

In some embodiments, B is

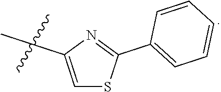 [SMILES: *c1csc(-c2ccccc2)n1]

In some embodiments, B is

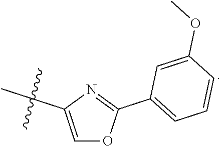 [SMILES: *c1coc(-c2cccc(OC)c2)n1]

In some embodiments, B is

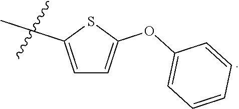 [SMILES: *c1ccc(Oc2ccccc2)s1]

In some embodiments, B is

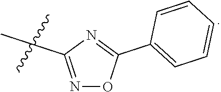 [SMILES: *c1noc(-c2ccccc2)n1]

In some embodiments, B is

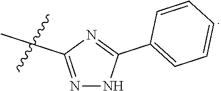 [SMILES: *c1n[nH]c(-c2ccccc2)n1]

In some embodiments, B is

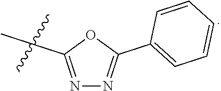 [SMILES: *c1nnc(-c2ccccc2)o1]

In some embodiments, B is

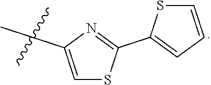 [SMILES: *c1csc(-c2cccs2)n1]

In some embodiments, B is

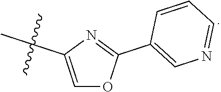 [SMILES: *c1coc(-c2cccnc2)n1]

In some embodiments, B is

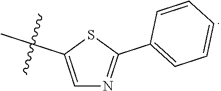 [SMILES: *c1cnc(-c2ccccc2)s1]

In some embodiments, B is

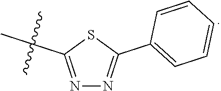 [SMILES: *c1nnc(-c2ccccc2)s1]

In some embodiments, B is

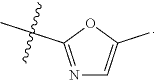 [SMILES: *c1ncc(C)o1]

In some embodiments, B is

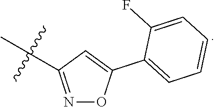 [SMILES: *c1cc(-c2ccccc2F)on1]

In some embodiments, B is

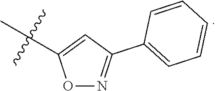 [SMILES: *c1cc(-c2ccccc2)no1]

In some embodiments, B is

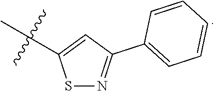 [SMILES: *c1cc(-c2ccccc2)ns1]

In some embodiments, B is

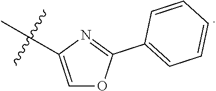 [SMILES: *c1coc(-c2ccccc2)n1]

In some embodiments, B is

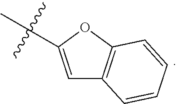 [SMILES: *c1cc2ccccc2o1]

In some embodiments, B is

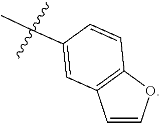 [SMILES: *c1ccc2occc2c1]

In some embodiments, B is

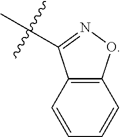 [SMILES: *c1noc2ccccc12]

In some embodiments, B is optionally substituted C₃-C₁₄ carbocyclyl or optionally substituted 5- to 14-membered heterocyclyl.

In some embodiments, the compound is a compound of Formula (V):

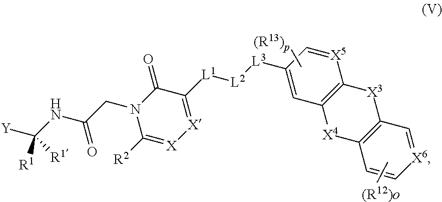

or a pharmaceutically acceptable salt thereof, in which each of X³ and X⁴ is independently a bond; 0; S; C(R′)₂, wherein each Rⁱ is independently H, OH, halo, optionally substituted C₁-C₆ alkyl, or optionally substituted C₁-C₆ alkoxy, or both Rⁱ combine to form oxo; NR^{j}, wherein R^{j} is H or C₁-C₆ alkyl; or SO₂; X⁵ is CH, CR¹³, or N; X⁶ is CH, CR¹², or N; o is selected from 0, 1, 2, and 3; p is selected from 0, 1, and 2; each R¹² and R¹³ is independently halo, optionally substituted C₁-C₆ alkyl, optionally substituted C₁-C₆ alkoxy, or optionally substituted C₃-C₈ cycloalkyl; and all other variables are as defined for Formula (I).

In some embodiments, the compound is a compound of Formula (VA):

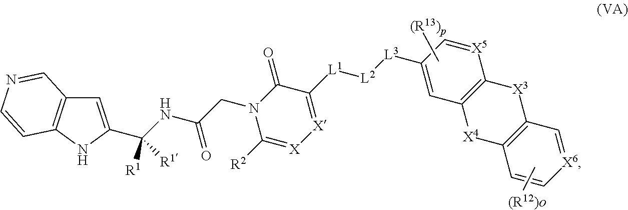

or a pharmaceutically acceptable salt thereof, in which all variables are as defined for Formula (V).

In some embodiments, the compound is a compound of Formula (VB):

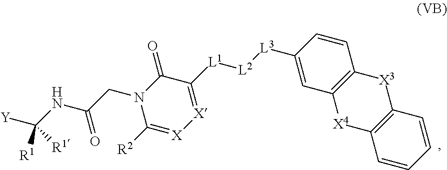

or a pharmaceutically acceptable salt thereof, in which all variables are as defined for Formula (V).

In some embodiments, the compound is a compound of formula (VC):

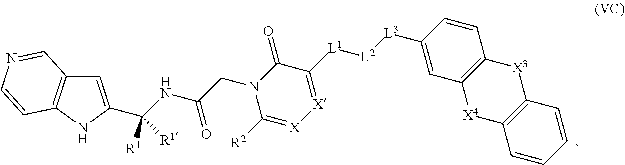

or a pharmaceutically acceptable salt thereof, in which all variables are as defined for Formula (V).

In some embodiments, the compound is a compound of Formula (VI):

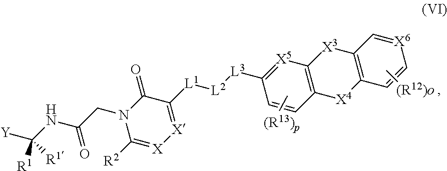

or a pharmaceutically acceptable salt thereof, in which each of X³ and X⁴ is independently a bond; 0; S; C(R′)₂, wherein each Rⁱ is independently H, OH, halo, optionally substituted C₁-C₆ alkyl, or optionally substituted C₁-C₆ alkoxy, or both Rⁱ combine to form oxo; NR^{j}, wherein R^{j} is H or C₁-C₆ alkyl; or SO₂; X⁵ is OH, OR¹³, or N; X⁶ is OH, OR¹², or N; o is selected from 0, 1, 2, and 3; p is selected from 0, 1, and 2; each R¹² and R¹³ is independently halo, optionally substituted C₁-C₆ alkyl, optionally substituted C₁-C₆ alkoxy, or optionally substituted C₃-C₈ cycloalkyl; and all other variables are as defined for Formula (I).

In some embodiments, the compound is a compound of Formula (VIA):

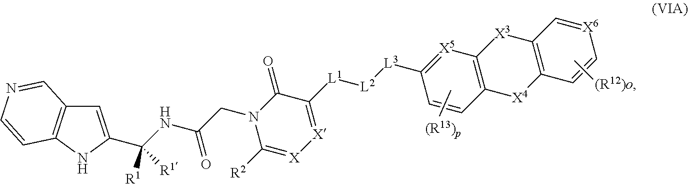

or a pharmaceutically acceptable salt thereof, in which all variables are as defined for Formula (V).

In some embodiments, the compound is a compound of Formula (VIB):

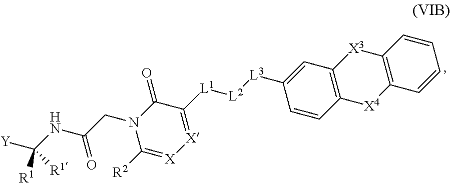

or a pharmaceutically acceptable salt thereof, in which all variables are as defined for Formula (VI).

In some embodiments, the compound is a compound of Formula (VIC):

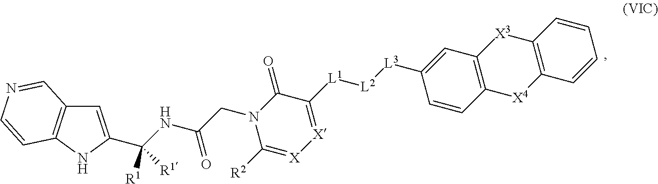

or a pharmaceutically acceptable salt thereof, in which all variables are as defined for Formula (VI).

In some embodiments, X³ is a bond. In some embodiments, X³ is O. In some embodiments, X³ is C(R′)₂. In some embodiments, X³ is CF₂. In some embodiments, X³ is S.

In some embodiments, X⁴ is a bond. In some embodiments, X⁴ is O. In some embodiments, X⁴ is C(R′)₂. In some embodiments, X³ is CF₂. In some embodiments, X⁴ is S.

In some embodiments, B is

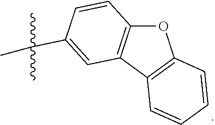 [SMILES: *c1ccc2oc3ccccc3c2c1]

In some embodiments, B is

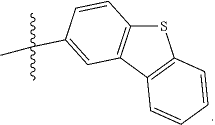 [SMILES: *c1ccc2sc3ccccc3c2c1]

In some embodiments, B is

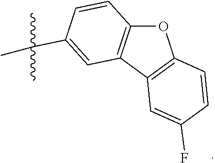 [SMILES: *c1ccc2oc3ccc(F)cc3c2c1]

In some embodiments, B is

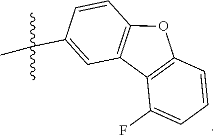 [SMILES: *c1ccc2oc3cccc(F)c3c2c1]

In some embodiments, B is

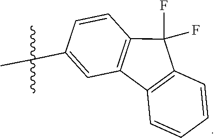 [SMILES: *c1ccc2c(c1)-c1ccccc1C2(F)F]

In some embodiments, B is

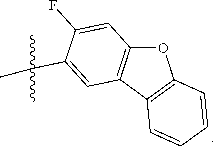 [SMILES: *c1cc2c(cc1F)oc1ccccc12]

In some embodiments, B is

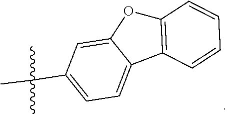 [SMILES: *c1ccc2c(c1)oc1ccccc12]

In some embodiments, B is

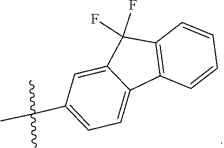 [SMILES: *c1ccc2c(c1)C(F)(F)c1ccccc1-2]

In some embodiments, B is optionally substituted C₃-C₁₄ carbocyclyl. In some embodiments, B is optionally substituted C₃-C₈ cycloalkyl. In some embodiments, B is

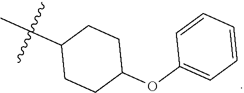 [SMILES: *C1CCC(Oc2ccccc2)CC1]

In some embodiments, B is

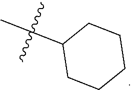 [SMILES: *C1CCCCC1]

In some embodiments, B is

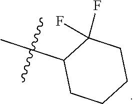 [SMILES: *C1CCCCC1(F)F]

In some embodiments, B is

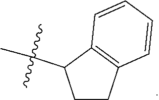 [SMILES: *C1CCc2ccccc21]

In some embodiments, B is optionally substituted 5- to 14-membered heterocyclyl. In some embodiments, B is

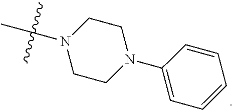 [SMILES: *N1CCN(c2ccccc2)CC1]

In some embodiments, B is

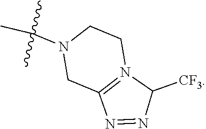

In some embodiments, B is

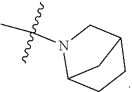 [SMILES: *N1CC2CCC1C2]

In some embodiments, B is

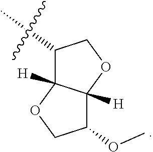

In some embodiments, B is

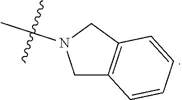 [SMILES: *N1Cc2ccccc2C1]

In some embodiments, B is

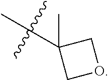 [SMILES: *C1(C)COC1]

In some embodiments, B is

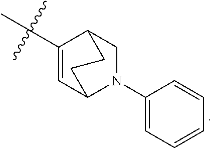 [SMILES: *C1=CC2CCC1CN2c1ccccc1]

In some embodiments, B is

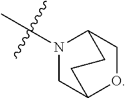 [SMILES: *N1CC2CCC1CO2]

In some embodiments, B is

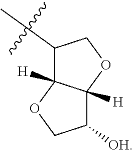 [SMILES: *C1CO[C@@H]2[C@H](O)CO[C@H]12]

In some embodiments, Y is

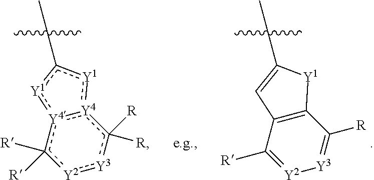

In some embodiments, Y² is N. In some embodiments, Y² is CR^{e}. In some embodiments, Y² is CH. In some embodiments, Y³ is N. In some embodiments, Y³ is CR^{e}. In some embodiments, Y³ is CH. In some embodiments, Y¹ is NR^{d}. In some embodiments, Y¹ is NH. In some embodiments, Y¹ is S.

In some embodiments, the compound is a compound of Formula (VII):

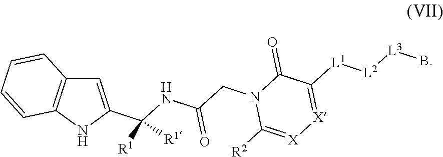

Or a pharmaceutically acceptable salt thereof.

In some embodiments, Y is

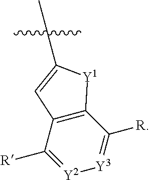

In some embodiments, Y¹ is S. In some embodiments, Y² is NRₑ. In some embodiments, Y² is NH. In some embodiments, Y² is C(R^{e})₂. In some embodiments, Y² is NH₂. In some embodiments, Y³ is NR^{e}. In some embodiments, Y³ is NH. In some embodiments, Y³ is C(R^{e})₂. In some embodiments, Y³ is CH₂.

In some embodiments, R is H. In some embodiments, R′ is H.

In some embodiments, Y is

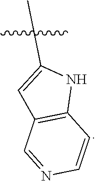 [SMILES: *c1cc2cnccc2[nH]1]

In some embodiments, Y is

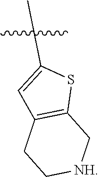 [SMILES: *c1cc2c(s1)CNCC2]

In some embodiments, Y is

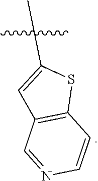 [SMILES: *c1cc2cnccc2s1]

In some embodiments, Y is

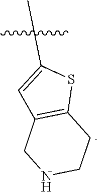 [SMILES: *c1cc2c(s1)CCNC2]

In some embodiments, Y is

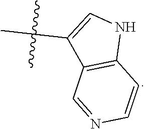 [SMILES: *c1c[nH]c2ccncc12]

In some embodiments, Y is

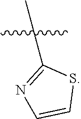 [SMILES: *c1nccs1]

In another aspect, the present disclosure provides a compound of Table 1, or a pharmaceutically acceptable salt thereof.

In some embodiments of any of the aspects provided herein, the compounds of the present disclosure have complement C1 esterase (C1s) inhibiting activity.

In some embodiments, a compound of the present disclosure is a compound of formula (II), (IIA), (III), (IIIA), (IV), (IVA), (V), (VA), (VB), (VC), (VI), (VIA), (VIB), (VIC), or (VII).

In some embodiments, a compound of the present disclosure is a compound of Table 1.

### Pharmaceutical Compositions

A pharmaceutical composition of the disclosure contains one or more of the compounds disclosed herein (e.g., one or more of the compounds of formulas (I), (I′), (II), (IIA), (III), (IIIA), (IV), (IVA), (V), (VA), (VB), (VC), (VI), (VIA), (VIB), (VIC), and (VII), and Table 1) as the therapeutic compound. In addition to a therapeutically effective amount of the compound, the pharmaceutical compositions also contain a pharmaceutically acceptable excipient, which can be formulated by methods known to those skilled in the art. In some embodiments, the pharmaceutical compositions for treating cancer contain one or more of the compounds disclosed herein (e.g., one or more of the compounds of formulas (I), (I′), (II), (IIA), (III), (IIIA), (IV), (IVA), (V), (VA), (VB), (VC), (VI), (VIA), (VIB), (VIC), and (VII), and Table 1) may be formulated and/or administered with or without other therapeutics for a particular condition. Examples of such therapeutics (second therapeutic agents) are described herein.

The compounds disclosed herein (e.g., the compounds of formulas (I), (I′), (II), (IIA), (III), (IIIA), (IV), (IVA), (V), (VA), (VB), (VC), (VI), (VIA), (VIB), (VIC), and (VII), and Table 1) may be used in the form of free base or in the form of salts. All forms are within the scope of the disclosure.

Exemplary routes of administration of the pharmaceutical compositions (or the compounds of the composition) include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. In some embodiments, a compound of the present disclosure (e.g., a compound of formulas (I), (I′), (II), (IIA), (III), (IIIA), (IV), (IVA), (V), (VA), (VB), (VC), (VI), (VIA), (VIB), (VIC), or (VII), or Table 1) is formulated for oral administration.

### Formulations for Oral Administration

The pharmaceutical compositions of the present disclosure include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers; granulating and disintegrating agents; binding agents; and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent, or as soft gelatin capsules where the active ingredient is mixed with water or an oil medium Powders, granulates, and pellets may be prepared using the ingredients mentioned above under tablets and capsules in a conventional manner using, e.g., a mixer, a fluid bed apparatus or a spray drying equipment.

The liquid forms in which the compounds and compositions of the present disclosure can be incorporated for administration orally include aqueous solutions, suitably flavored syrups, aqueous or oil suspensions, and flavored emulsions with edible oils, as well as elixirs and similar pharmaceutical vehicles.

### Formulations for Parenteral Administration

The pharmaceutical compositions of the present disclosure can be administered in a pharmaceutically acceptable parenteral (e.g., intravenous, intramuscular, subcutaneous or the like) formulation as described herein. The pharmaceutical composition may also be administered parenterally in dosage forms or formulations containing conventional, non-toxic pharmaceutically acceptable carriers and adjuvants. In particular, formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the present disclosure may be dissolved or suspended in a parenterally acceptable liquid vehicle. Among acceptable vehicles and solvents that may be employed are water; water adjusted to a suitable pH by addition of an appropriate amount of hydrochloric acid, sodium hydroxide, or a suitable buffer; 1,3-butanediol; Ringer's solution; and isotonic sodium chloride solution. The aqueous formulation may also contain one or more preservatives. Additional information regarding parenteral formulations can be found, for example, in the United States Pharmacopeia-National Formulary (USP-NF), herein incorporated by reference in its entirety.

The parenteral formulation can be any of the five general types of preparations identified by the USP-NF as suitable for parenteral administration:
(1) “Drug Injection:” a liquid preparation that is a drug substance (e.g., a compound of the present disclosure), or a solution thereof;
(2) “Drug for Injection:” the drug substance (e.g., a compound of the present disclosure) as a dry solid that will be combined with the appropriate sterile vehicle for parenteral administration as a drug injection;
(3) “Drug Injectable Emulsion:” a liquid preparation of the drug substance (e.g., a compound of the present disclosure) that is dissolved or dispersed in a suitable emulsion medium;
(4) “Drug Injectable Suspension:” a liquid preparation of the drug substance (e.g., a compound of the present disclosure) suspended in a suitable liquid medium; and
(5) “Drug for Injectable Suspension:” the drug substance (e.g., a compound of the present disclosure) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropyl cellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 23^{rd} Ed., Adejare, Ed., Academic Press (2020) and in The United States Pharmacopeia and National Formulary (USP-NF 2021 Issues 1-3), published in 2021.

Formulations for parenteral administration may, for example, contain sterile water, saline, polyalkylene glycols (e.g., polyethylene glycol), oils of vegetable origin, or hydrogenated naphthalenes.

Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain, for example, lactose, or may be aqueous solutions containing, for example, polyoxyethylene-9-lauryl ether, glycocholate and deoxycholate, or may be oily solutions for administration in the form of nasal drops, or as a gel.

The dosage of the compounds described herein (e.g., the compounds of formulas (I), (I′), (II), (IIA), (III), (IIIA), (IV), (IVA), (V), (VA), (VB), (VC), (VI), (VIA), (VIB), (VIC), and (VII), and Table 1), and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the subject to be treated. One of skill in the art can determine the appropriate dosage based on the above factors. In general, satisfactory results may be obtained when the compounds described herein are administered to a human at a daily dosage of, for example, between 0.05 mg and 3000 mg (measured as the solid form). For example, the dose range may be 10-1000 mg (e.g., 50-800 mg).

Alternatively, the dosage amount can be calculated using the body weight of the patient. For example, the dose of a compound, or pharmaceutical composition thereof, administered to a patient may be 0.1-100 mg/kg. A dosage form containing a compound disclosed herein (e.g., a compound of formula (I), (I′), (II), (IIA), (III), (IIIA), (IV), (IVA), (V), (VA), (VB), (VC), (VI), (VIA), (VIB), (VIC), or (VII), or Table 1) can be administered, for example, once a day (QD), twice a day (BID), three times a day (TID), four times a day (QID), once every other day (Q2D), once every third day (Q3D), or any dosing schedule as needed,

### Uses of Active Compounds for Treatment of Selected Disorders

In one aspect, an effective amount of an active compound described herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1, or a pharmaceutically acceptable salt thereof) is used to treat a medical disorder which is an inflammatory or immune condition, a disorder mediated by the complement cascade (including a dysfunctional cascade) including a complement-related disorder or alternative complement pathway-related disorder, a disorder or abnormality of a cell that adversely affects the ability of the cell to engage in or respond to normal complement activity, or an undesired complement-mediated response to a medical treatment, such as surgery or other medical procedure or a pharmaceutical or biopharmaceutical drug administration, a blood transfusion, or other allogenic tissue or fluid administration.

In some embodiments, the disorder is an autoimmune disease. In some embodiments, the disorder is cancer. In some embodiments, the disorder is an infectious disease. In some embodiments, the disorder is an inflammatory disease. In some embodiments, the disorder is a hematological disease. In some embodiments, the disorder is an ischemia-reperfusion injury. In some embodiments, the disorder is an oculardisease. In some embodiments, the disorder is a renal disease. In some embodiments, the disorder is transplant rejection. In some embodiments, the disorder is antibody-mediated transplant rejection, e.g., acute antibody-mediated rejection. In some embodiments, the disorder is a vascular disease. In some embodiments, the disorder is a vasculitis disorder. In some embodiments, the disorder is a neurodegenerative disorder, e.g., a tauopathy.

In some embodiments, the disorder is a medical disorder of the central nervous system (CNS) or peripheral nervous system disorders involving complement activation. In some embodiments, the disorder is an acquired brain or spinal cord injury. In some embodiments, the disorder is ischemic-reperfusion injury. In some embodiments, the disorder is stroke. In some embodiments, the disorder is traumatic brain injury (TBI). In some embodiments, the disorder is spinal cord injury (SCI).

In some embodiments, the disorder is a neuroinflammatory disorder.

In some embodiments, the neuroinflammatory disorder is cranial arteritis. In some embodiments, the neuroinflammatory disorder is giant cell arteritis. In some embodiments, the neuroinflammatory disorder is Holmes-Adie syndrome. In some embodiments, the neuroinflammatory disorder is inclusion body myositis (IBM). In some embodiments, the neuroinflammatory disorder is meningitis. In some embodiments, the neuroinflammatory disorder is a neurologic paraneoplastic syndrome, e.g., Lambert-Eaton myasthenic syndrome, stiff-person syndrome, encephalomyelitis (inflammation of the brain and spinal cord), myasthenia gravis, cerebellar degeneration, limbic and/or brainstem encephalitis, neuromyotonia, opsoclonus (involving eye movement), or sensory neuropathy. In some embodiments, the neuroinflammatory disorder is polymyositis. In some embodiments, the neuroinflammatory disorder is transverse myelitis. In some embodiments, the neuroinflammatory disorder is vasculitis, e.g., temporal arteritis. In some embodiments, the neuroinflammatory disorder is arachnoiditis. In some embodiments, the neuroinflammatory disorder is Kinsbourne syndrome. In some embodiments, the neuroinflammatory disorder is opsoclonus myoclonus syndrome (OMS). In some embodiments, the neuroinflammatory disorder is Saint Vitus Dance or Sydenham's chorea (SD) disease.

In some embodiments, the disorder is Alzheimer's disease (AD). AD is characterized by two hallmark pathologies; amyloid-β (Aβ) plaques and neurofibrillary tangles comprising hyperphosphorylated tau. Recent studies have implicated complement in AD pathogenesis, including genome-wide association studies identifying single nucleotide polymorphisms (SNPs) associated with risk of late-onset AD in genes encoding complement proteins clusterin (CLU) and CR1 (CR1). See Carpanini et al., Therapeutic Inhibition of the Complement System in Diseases of the Central Nervous System, Front. Immunol., 4 Mar. 2019. Biomarker studies have also identified complement proteins and activation products in plasma and/or CSF that distinguish AD from controls and predict risk of progression to AD.

In some embodiments, the disorder is frontotemporal dementia. In some embodiments, the disorder is Pick's disease. In some embodiments, the disorder is sporadic frontotemporal dementia, e.g., frontotemporal dementia with Parkinsonism linked to chromosome 17. In some embodiments, progressive supranuclear palsy (PSP). In some embodiments, corticobasal degeneration (CBD). In some embodiments, the disorder is subacute sclerosing panencephalitis.

In some embodiments, the disorder is amyotrophic lateral sclerosis (ALS). ALS is caused by progressive loss of upper and lower (a) motor neurons resulting in denervation of neuromuscular junctions in the peripheral nervous system, progressive muscle weakness, atrophy, spasticity, respiratory failure, and ultimately paralysis and death. Recent studies have shown increased C1q protein in motor cortex and spinal cord of ALS post-mortem tissue; C3 activation fragments and TCC in areas of pathology; C4d and TCC staining of degenerating neurons and glia in ALS motor cortex and spinal cord, and C5aR1 upregulation in areas of pathology. C3d and C4d have been found on oligodendroglia and degenerating neurites, surrounded by CR4-positive microglia, in spinal cord and motor cortex, and C1q, C3, and TCC have been shown to be present on motor endplates in intercostal muscles in ALS donors even early in the disease process. See Carpanini et al., Therapeutic Inhibition of the Complement System in Diseases of the Central Nervous System, Front. Immunol., 4 Mar. 2019.

In some embodiments, the disorder is Parkinson's disease (PD). PD is characterized by loss of dopaminergic neurons in the substantia nigra and deposits of the protein α-synuclein that form the pathological hallmarks of the disease, Lewy bodies. Patients present with resting tremor, bradykinesia, and rigidity. Complement activation has been associated with α-synuclein and Lewy bodies in Parkinson's disease; in vitro studies have demonstrated that the disease-associated splice variant α-synuclein 112, but not the full-length protein, cause activation of complement. In vivo, C3d, C4d, C7 and C9 localization in Lewy bodies has been reported. More recently, deposition of iC3b and C9 in Lewy bodies and melanized neurons has been reported, and iC3b immunoreactivity has been shown to be increased with normal ageing and was further elevated in PD vs. age-matched controls. Furthermore, correlation between the ratios of C3/AP42 or FH/AP42 in CSF and severity of Parkinson's disease motor and cognitive symptoms has been shown. See Carpanini et al., Therapeutic Inhibition of the Complement System in Diseases of the Central Nervous System, Front. Immunol., 4 Mar. 2019. In some embodiments, the subject to be treated suffers from Parkinson's Disease with dementia (PDD).

In some embodiments, the disorder is Huntington's disease (HD). HD is an autosomal dominant, inherited neurodegenerative disease characterized by progressive motor symptoms, psychiatric disturbances, and dementia. It is caused by expansion of a three-base-pair (CAG) repeat (39-121 repeats vs. normal range 8-39 repeats) in exon 1 of the HTT gene that translates into a polyglutamine tract at the N-terminus of the protein. This results in a polyglutamine length-dependent misfolding and accumulation of huntingtin protein in the striatum and cortex (layers 3, 5, and 6) followed by neuronal loss in these areas which spreads to the hippocampus. It has been shown that neurons, astrocytes, and myelin sheaths in the HD caudate and striatum were immunoreactive for C1q, C4, C3 and neo-epitopes in iC3b and TCC. Expression of mRNA encoding early complement components C1q (c-chain), C1 r, C3, and C4, complement regulators C1INH, Clusterin, MCP, DAF and CD59, and complement receptors C3a and C5a, have been shown to be upregulated in the HD striatum, see Carpanini et al., Therapeutic Inhibition of the Complement System in Diseases of the Central Nervous System, Front. Immunol., 4 Mar. 2019.

In some embodiments, the disorder is argyrophilic grain dementia. In some embodiments, the disorder is British type amyloid angiopathy. In some embodiments, the disorder is cerebral amyloid angiopathy. In some embodiments, the disorder is Creutzfeldt-Jakob disease. In some embodiments, the disorder is dementia pugilistica. In some embodiments, the disorder is diffuse neurofibrillary tangles with calcification. In some embodiments, the disorder is Down's syndrome. In some embodiments, the disorder is frontotemporal lobar degeneration. In some embodiments, the disorder is Gerstmann-Straussler-Scheinker disease. In some embodiments, the disorder is Hallervorden-Spatz disease. In some embodiments, the disorder is inclusion body myositis. In some embodiments, the disorder is multiple system atrophy (MSA). In some embodiments, the disorder is myotonic dystrophy. In some embodiments, the disorder is Niemann-Pick disease type C. In some embodiments, the disorder is non-Guamanian motor neuron disease with neurofibrillary tangles. In some embodiments, the disorder is postencephalitic parkinsonism. In some embodiments, the disorder is prion protein cerebral amyloid angiopathy. In some embodiments, the disorder is progressive subcortical gliosis. In some embodiments, the disorder is progressive supranuclear palsy. In some embodiments, the disorder is subacute sclerosing panencephalitis. In some embodiments, the disorder is Tangle only dementia. In some embodiments, the disorder is multi-infarct dementia. In some embodiments, the disorder is ischemic stroke. In some embodiments, the disorder is chronic traumatic encephalopathy (CTE).

In some embodiments, the disorder is a hereditary motor and sensory neuropathy (HMSN). In some embodiments, the HMSN is Charcot-Marie-Tooth (CMT) disease. In some embodiments, the HSMN is Charcot-Marie-Tooth disease type 1A or type 1B. In some embodiments, the HSMN is Charcot-Marie-Tooth disease type 2. In some embodiments, the HSMN is Dejerine-Sottas disease (Charcot-Marie-Tooth type 3). In some embodiments, the HSMN is Refsum disease. In some embodiments, the HSMN is Charcot-Marie-Tooth with pyramidal features. In some embodiments, the HSMN is Charcot-Marie-Tooth type 6. In some embodiments, the HSMN is HMSN+retinitis pigmentosa.

In some embodiments, the disorder is Churg-Strauss syndrome. In some embodiments, the disorder is peripheral artery disease (PAD). In some embodiments, the disorder is myasthenia gravis, e.g., myasthenia gravis with CNS involvement. In some embodiments, the disorder is dementia with Lewy bodies. In some embodiments, the disorder is prion disease. In some embodiments, the disorder is Behcet's Disease. In some embodiments, the disorder is congenital myasthenia. In some embodiments, the disorder is subacute sclerosing panencephalitis (SSPE).

In some embodiments, the disorder is a demyelinating disease. In some embodiments, the disorder is demyelinating myelinoclastic disease. In some embodiments, the disorder is demyelinating leukodystrophic disease.

In some embodiments, the demyelinating myelinoclastic disease is multiple sclerosis (MS). Multiple sclerosis (MS) is the most common cause of neurological disability in young adults in northern European-Caucasian populations, with an approximate lifetime risk of one in 400. C3 has been shown to be deposited in the brains of MS patients. T-cell clone (TCC) has been shown to be in association with capillary endothelial cells, predominantly within plaques and adjacent white matter. Localization of C activation to areas of active myelin destruction has also been shown, with TCC deposited exclusively in such areas. C3d has been shown to be deposited in association with short segments of disrupted myelin in plaques with low-grade active demyelination and provides evidence for a C contribution to disease progression as well as acute inflammation. See Ingram et al., Complement in multiple sclerosis: its role in disease and potential as a biomarker. Clin Exp Immunol. 2009 February; 155(2):128-39.

In some embodiments, the demyelinating myelinoclastic disease is neuromyelitis optica (NMO). Neuromyelitis optica (NMO) is an inflammatory demyelinating disease affecting predominantly the optic nerves and spinal cord. Traditionally seen as a variant of MS, it has been redefined recently according to new criteria using a combination of phenotypic subtyping along with a newly developed biomarker of disease, NMO-immunoglobulin G (IgG) (reported sensitivity of 58-76% and specificity of 85-99% for NMO). NMO patients have higher levels of C3a and anti-C1q antibodies than healthy controls. C3a levels correlated with disease activity, neurological disability and aquaporin-4 IgG. Nytrova et al. J Neuroimmunol. 2014 Sep. 15; 274(1-2):185-91.

In some embodiments, the demyelinating myelinoclastic disease is neuromyelitis optica spectrum disorder (NMOSD). In some embodiments, the demyelinating myelinoclastic disease is idiopathic inflammatory demyelinating diseases (IIDD). In some embodiments, the demyelinating myelinoclastic disease is anti-NMDA receptor encephalitis. In some embodiments, the demyelinating myelinoclastic disease is acute disseminated encephalomyelitis. In some embodiments, the demyelinating myelinoclastic disease is anti-MOG autoimmune encephalomyelitis. In some embodiments, the demyelinating myelinoclastic disease is chronic relapsing inflammatory optic neuritis (CRION). In some embodiments, the demyelinating myelinoclastic disease is acute disseminated encephalomyelitis (ADEM). In some embodiments, the demyelinating myelinoclastic disease is immune-mediated encephalomyelitis. In some embodiments, the demyelinating myelinoclastic disease is progressive multifocal leukoencephalopathy (PML). In some embodiments, the demyelinating myelinoclastic disease is McDonalds-positive multiple sclerosis. In some embodiments, the demyelinating myelinoclastic disease is acute hemorrhagic leukoencephalitis. In some embodiments, the demyelinating myelinoclastic disease is Rasmussen's Encephalitis. In some embodiments, the demyelinating myelinoclastic disease is Marburg multiple sclerosis. In some embodiments, the demyelinating myelinoclastic disease is pseudotumefactive or tumefactive multiple sclerosis. In some embodiments, the demyelinating myelinoclastic disease is Balo concentric sclerosis. In some embodiments, the demyelinating myelinoclastic disease is diffuse myelinoclastic sclerosis. In some embodiments, the demyelinating myelinoclastic disease is solitary sclerosis. In some embodiments, the demyelinating myelinoclastic disease is multiple sclerosis with cavitary lesions. In some embodiments, the demyelinating myelinoclastic disease is myelocortical multiple sclerosis (MCMS). In some embodiments, the demyelinating myelinoclastic disease is atypical optic-spinal multiple sclerosis. In some embodiments, the demyelinating myelinoclastic disease is pure spinal multiple sclerosis. In some embodiments, the demyelinating myelinoclastic disease is HLA DRB3*02:02 multiple sclerosis. In some embodiments, the demyelinating myelinoclastic disease is autoimmune GFAP astrocytopathy. In some embodiments, the demyelinating myelinoclastic disease is chronic inflammatory demyelinating polyneuropathy (CIDP). In some embodiments, the demyelinating myelinoclastic disease is Guillain-Barre syndrome (acute or chronic). In some embodiments, the demyelinating myelinoclastic disease is progressive inflammatory neuropathy. In some embodiments, the demyelinating myelinoclastic disease is Lewis-Sumner Syndrome. In some embodiments, the demyelinating myelinoclastic disease is combined central and peripheral demyelination (CCPD). In some embodiments, the demyelinating myelinoclastic disease is Bickerstaff brainstem encephalitis. In some embodiments, the demyelinating myelinoclastic disease is Fisher syndrome. In some embodiments, the demyelinating myelinoclastic disease is trigeminal neuralgia. In some embodiments, the demyelinating myelinoclastic disease is NMDAR anti-NMDA receptor encephalitis. In some embodiments, the demyelinating myelinoclastic disease is primary progressive MS (PPMS). In some embodiments, the demyelinating myelinoclastic disease is OPA1 variant multiple sclerosis. In some embodiments, the demyelinating myelinoclastic disease is KIR4.1 multiple sclerosis. In some embodiments, the demyelinating myelinoclastic disease is aquaporin-related multiple sclerosis. In some embodiments, the demyelinating myelinoclastic disease is chronic cerebrospinal venous insufficiency (CCSVI or CCVI). In some embodiments, the demyelinating myelinoclastic disease is diffuse sclerosis. In some embodiments, the demyelinating myelinoclastic disease is Schilder's disease.

In certain aspects, the disorder to be treated is a demyelinating leukodystrophic disease. In some embodiments, the demyelinating leukodystrophic disease is myelitis. In some embodiments, the demyelinating leukodystrophic disease is central pontine myelinolysis (CPM). In some embodiments, the demyelinating leukodystrophic disease is extrapontine myelinolysis. In some embodiments, the demyelinating leukodystrophic disease is tabes dorsalis. In some embodiments, the demyelinating leukodystrophic disease is progressive multifocal leukoencephalopathy. In some embodiments, the demyelinating leukodystrophic disease is leukoencephalopathy with vanishing white matter. In some embodiments, the demyelinating leukodystrophic disease is leukoencephalopathy with neuroaxonal spheroids. In some embodiments, the demyelinating leukodystrophic disease is reversible posterior leukoencephalopathy syndrome. In some embodiments, the demyelinating leukodystrophic disease is megalencephalic leukoencephalopathy with subcortical cysts. In some embodiments, the demyelinating leukodystrophic disease is megalencephalic leukoencephalopathy with subcortical cysts 1. In some embodiments, the demyelinating leukodystrophic disease is hypertensive leukoencephalopathy. In some embodiments, the demyelinating leukodystrophic disease is metachromatic leukodystrophy. In some embodiments, the demyelinating leukodystrophic disease is Krabbe disease. In some embodiments, the demyelinating leukodystrophic disease is Canavan disease. In some embodiments, the demyelinating leukodystrophic disease is X-linked adrenoleukodystrophy. In some embodiments, the demyelinating leukodystrophic disease is Alexander disease. In some embodiments, the demyelinating leukodystrophic disease is cerebrotendinous xanthomatosis. In some embodiments, the demyelinating leukodystrophic disease is Pelizaeus-Merzbacher disease. In some embodiments, the demyelinating leukodystrophic disease is Refsum disease.

In some embodiments, an effective amount of an active compound described herein, or a pharmaceutically acceptable salt thereof, is used to treat Buerger's disease, also known as thromboangiitis obliterans.

In some embodiments, an effective amount of an active compound described herein, or a pharmaceutically acceptable salt thereof, is used to treat giant cell arteritis.

In some embodiments, an effective amount of an active compound described herein, or a pharmaceutically acceptable salt thereof, is used to treat Raynaud's disease.

In certain aspects, the disorder to be treated is a demyelinating disease of the peripheral nervous system. In some embodiments, the demyelinating disease of the peripheral nervous system is anti-MAG peripheral neuropathy. In some embodiments, the demyelinating disease of the peripheral nervous system is hereditary neuropathy with liability to pressure palsy. In some embodiments, the demyelinating disease of the peripheral nervous system is a copper deficiency-associated condition (e.g., peripheral neuropathy, myelopathy, or rarely optic neuropathy).

In some embodiments, an effective amount of an active compound described herein, or a pharmaceutically acceptable salt thereof, is used to treat transverse myelitis.

In certain aspects, the disorder to be treated is a peripheral neuropathy. In some embodiments, the peripheral neuropathy is a mononeuropathy. In some embodiments, the neuropathy is a polyneuropathy. In some embodiments, the polyneuropathy is distal axonopathy, diabetic neuropathy, a demyelinating polyneuropathy, small fiber peripheral neuropathy, mononeuritis multiplex, polyneuritis multiplex, autonomic neuropathy, or neuritis.

In some embodiments, an effective amount of an active compound described herein, or a pharmaceutically acceptable salt thereof, is used to treat multifocal motor neuropathy.

In some embodiments, an effective amount of an active compound described herein, or a pharmaceutically acceptable salt thereof, is used to treat an autoimmune vascular disease. In some embodiments, the autoimmune vascular disease is vasculitis. In some embodiments, the vasculitis includes, but is not limited to, autoimmune inflammatory vasculitis, Cutaneous small-vessel vasculitis, Granulomatosis with polyangiitis, Eosinophilic granulomatosis with polyangiitis, Behçet's disease, Kawasaki disease, Buerger's disease, and “Limited” granulomatosis with polyangiitis vasculitis.

In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein is used to treat an arteritis. In some embodiments, the arteritis is giant cell arteritis. In some embodiments, the arteritis is Takayasu arteritis. In some embodiments, the arteritis is temporal arteritis. In some embodiments, the arteritis is polyarteritis nodosa.

In some embodiments, a method for the treatment of a glomerulonephritis is provided. In some embodiment, the glomerulonephritis is membranoproliferative glomerulonephritis (MPGN). In some embodiments, the MPGN is MPGN Type I. In some embodiments, the MPGN is MPGN Type II. In some embodiments, the MPGN is MPGN Type III. In some embodiments, the MPGN is C3 glomerulonephritis (C3G). In some embodiments, the MPGN is dense deposit disease (DDD). In some embodiments, the MPGN is a C4 deposition disorder.

In some embodiments, the glomerulonephritis is IC-MPGN. In some embodiments, the glomerulonephritis is a membranous glomerulonephritis. In some embodiments, the glomerulonephritis is IgA nephropathy. In some embodiments, the glomerulonephritis is post-infectious glomerulonephritis. In some embodiments, the glomerulonephritis is a rapidly progressive glomerulonephritis, for example Type I (Goodpasture syndrome), Type II, or Type III rapidly progressive glomerulonephritis.

In some embodiments, a method for the treatment of paroxysmal nocturnal hemoglobinuria (PNH) is provided that includes the administration of an effective amount of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof to a subject, optionally in a pharmaceutically acceptable composition.

In some embodiments, a method for the treatment of hereditary angioedema (HAE) is provided that includes the administration of an effective amount of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof to a subject, optionally in a pharmaceutically acceptable composition. Mutations in the SERPING1 gene cause hereditary angioedema type I and type II. Hereditary angioedema is a disorder characterized by recurrent episodes of severe swelling (angioedema). The most common areas of the body to develop swelling are the limbs, face, intestinal tract, and airway. The SERPING1 gene provides instructions for making the C1 inhibitor protein, which is important for controlling inflammation. C1 inhibitor blocks the activity of certain proteins that promote inflammation. Mutations that cause hereditary angioedema type I lead to reduced levels of C1 inhibitor in the blood, while mutations that cause type II result in the production of a C1 inhibitor that functions abnormally. Without the proper levels of functional C1 inhibitor, excessive amounts of a protein fragment (peptide) called bradykinin are generated. Bradykinin promotes inflammation by increasing the leakage of fluid through the walls of blood vessels into body tissues. Excessive accumulation of fluids in body tissues causes the episodes of swelling seen in individuals with hereditary angioedema type I and type II.

In some embodiments, a method for the treatment of cold agglutinin disease (CAD) is provided that includes the administration of an effective amount of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof to a subject, optionally in a pharmaceutically acceptable composition. CAD is a rare autoimmune hemolytic condition with potentially serious acute and chronic consequences that are driven by C1 activation of the classical complement pathway.

In some embodiments, a method for the treatment of atypical hemolytic uremic syndrome (aHUS) is provided that includes the administration of an effective amount of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof to a subject, optionally in a pharmaceutically acceptable composition. Atypical hemolytic-uremic syndrome is a disease that primarily affects kidney function. Atypical hemolytic uremic syndrome, which can occur at any age, causes abnormal blood clots (thrombi) to form in small blood vessels in the kidneys. These clots can cause serious medical problems if they restrict or block blood flow. Atypical hemolytic-uremic syndrome is characterized by three major features related to abnormal clotting: hemolytic anemia, thrombocytopenia, and kidney failure.

In another embodiment, a method for the treatment of wet or dry age-related macular degeneration (AMD) in a subject is provided that includes the administration of an effective amount of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof to a subject, optionally in a pharmaceutically acceptable composition. In another embodiment, a method for the treatment of rheumatoid arthritis in a subject is provided that includes the administration of an effective amount of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof to a subject, optionally in a pharmaceutically acceptable composition.

In another embodiment, a method for the treatment of multiple sclerosis in a subject is provided that includes the administration of an effective amount of a compound disclosed herein (e.g., any one of the compounds of formulas (I), (I′), (II), (IIA), (III), (IIIA), (IV), (IVA), (V), (VA), (VB), (VC), (VI), (VIA), (VIB), (VIC), and (VII), and Table 1) or a pharmaceutically acceptable salt thereof to a subject, optionally in a pharmaceutically acceptable composition.

The active compounds or pharmaceutically acceptable salts thereof disclosed herein, are also useful for administration in combination (in the same or a different dosage form) or alternation with a second pharmaceutical agent for use in ameliorating or reducing a side effect of the second pharmaceutical agent.

For example, in some embodiments, the active compound may be used in combination with an adoptive cell-transfer therapy to reduce an inflammatory response associated with such therapy, for example, a cytokine mediated response such as cytokine response syndrome.

In some embodiments, the adoptive cell-transfer therapy is a chimeric antigen receptor T-Cell (CAR T), or a dendritic cell used to treat a hematologic or solid tumor, for example, a B-cell related hematologic cancer.

In some embodiments, the hematologic or solid tumor is acute lymphoblastic leukemia (ALL), acute myeloid leukemia (AML), non-Hodgkin's lymphoma, chronic lymphocytic leukemia (CLL), pancreatic cancer, glioblastoma, or a cancer that expresses CD19.

In some embodiments, the adoptive cell-transfer therapy is a non-engineered T-cell therapy, in which the T-cells have been activated and/or expanded to one or more viral or tumor antigens. In some embodiments, the associated inflammatory response is a cytokine mediated response.

In some embodiments, the second pharmaceutical agent is a cell that has been transformed to express a protein, in which the protein in the subject is mutated or otherwise has impaired function. In some embodiments, the transformed cell includes a CRISPR gene.

Another embodiment is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or a pharmaceutically acceptable salt thereof, optionally in a pharmaceutically acceptable composition to a subject to treat an ocular, pulmonary, gastrointestinal, or other disorder.

In other embodiments of the disclosure, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) provided herein can be used to treat or prevent a disorder in a subject mediated by complement. As examples, the disclosure includes methods to treat or prevent complement associated disorders that are induced by antibody-antigen interactions, a component of an immune or autoimmune disorder or by ischemic injury. The disclosure also provides methods to decrease inflammation or an immune response, including an autoimmune response, where mediated or affected by the classical complement pathway.

In some embodiments, the disorder is selected from fatty liver and conditions stemming from fatty liver, such as nonalcoholic steatohepatitis (NASH), liver inflammation, cirrhosis, and liver failure. In some embodiments of the present disclosure, a method is provided for treating fatty liver disease in a subject by administering an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In another embodiment, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein is used to modulate an immune response prior to or during surgery or other medical procedure. One non-limiting example is use in connection with acute or chronic graft versus subject disease, which is a common complication as a result of organ transplantation, allogeneic tissue transplant, and can also occur as a result of a blood transfusion.

In some embodiments, the present disclosure provides a method of treating dermatomyositis by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating amyotrophic lateral sclerosis by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating abdominal aortic aneurysm, hemodialysis complications, hemolytic anemia, or hemodialysis by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In another embodiment, a method is provided for the treatment or prevention of cytokine or inflammatory reactions in response to the administration of pharmaceutical or biotherapeutic (e.g., CAR T-cell therapy or monoclonal antibody therapy) in a subject by administering an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein. Various types of cytokine or inflammatory reactions may occur in response to a number of factors, such as the administrations of biotherapeutics.

In some embodiments, the cytokine or inflammatory reaction is cytokine release syndrome. In some embodiments, the cytokine or inflammatory reaction is tumor lysis syndrome (which also leads to cytokine release). Symptoms of cytokine release syndrome range from fever, headache, and skin rashes to bronchospasm, hypotension, and even cardiac arrest. Severe cytokine release syndrome is described as a cytokine storm and can be fatal.

Fatal cytokine storms have been observed in response to infusion with several monoclonal antibody therapeutics. See, Abramowicz D, et al. “Release of tumor necrosis factor, interleukin-2, and gamma-interferon in serum after injection of OKT3 monoclonal antibody in kidney transplant recipients” Transplantation (1989) 47(4):606-8; Chatenoud L, et al. “In vivo cell activation following OKT3 administration. Systemic cytokine release and modulation by corticosteroids” Transplantation (1990) 49(4):697-702; and Lim L C, Koh L P, and Tan P. “Fatal cytokine release syndrome with chimeric anti-CD20 monoclonal antibody rituximab in a 71-year-old patient with chronic lymphocytic leukemia” J. Clin Oncol. (1999) 17(6):1962-3.

Also contemplated herein, is the use of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to mediate an adverse immune response in patients receiving bi-specific T-cell engagers (BiTE). A bi-specific T-cell engager directs T-cells to target and bind with a specific antigen on the surface of a cancer cell. For example, Blinatumomab (Amgen), a BiTE has recently been approved as a second line therapy in Philadelphia chromosome-negative relapsed or refractory acute lymphoblastic leukemia. Blinatumomab is given by continuous intravenous infusion in 4-week cycles. The use of BiTE agents has been associated with adverse immune responses, including cytokine release syndrome. The most significantly elevated cytokines in the CRS associated with ACT include IL-10, IL-6, and IFN-γ (Klinger et al., Immunopharmacologic response of patients with B-lineage acute lymphoblastic leukemia to continuous infusion of T cell-engaging CD19/CD3-bispecific BiTE antibody blinatumomab. Blood (2012) 119:6226-6233).

In another embodiment, the disorder is episcleritis, idiopathic episcleritis, anterior episcleritis, or posterior episcleritis. In some embodiments, the disorder is idiopathic anterior uveitis, HLA-B27 related uveitis, herpetic keratouveitis, Posner Schlossman syndrome, Fuch's heterochromic iridocyclitis, or cytomegalovirus anterior uveitis.

In some embodiments, the present disclosure provides a method of treating an IC-MPGN by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating a paroxysmal nocturnal hemoglobinuria (PNH) by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating a hereditary angioedema (HAE) by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating cold agglutinin disease (CAD) by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating atypical hemolytic syndrome (aHUS) by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating age-related macular degeneration (AMD) by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating rheumatoid arthritis by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating multiple sclerosis by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the present disclosure provides a method of treating myasthenia gravis by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), and (VII), or Table 1) or its salt or composition as described herein.

In some embodiments, the present disclosure provides a method of treating atypical hemolytic uremic syndrome (aHUS) by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In another embodiment, the present disclosure provides a method of treating a disorder as described below by administering to a subject in need thereof an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein, including: vitritis, sarcoidosis, syphilis, tuberculosis, or Lyme disease; retinal vasculitis, Eales disease, tuberculosis, syphilis, or toxoplasmosis; neuroretinitis, viral retinitis, or acute retinal necrosis; varicella zoster virus, herpes simplex virus, cytomegalovirus, Epstein-Barr virus, lichen planus, or Dengue-associated disease (e.g., hemorrhagic Dengue Fever); Masquerade syndrome, contact dermatitis, trauma induced inflammation, UVB induced inflammation, eczema, granuloma annulare, or acne.

In an additional embodiment, the disorder is selected from: acute myocardial infarction, aneurysm, cardiopulmonary bypass, dilated cardiomyopathy, complement activation during cardiopulmonary bypass operations, coronary artery disease, restenosis following stent placement, or percutaneous transluminal coronary angioplasty (PTCA); antibody-mediated transplant rejection, anaphylactic shock, anaphylaxis, allogenic transplant, humoral and vascular transplant rejection, graft dysfunction, graft-versus-subject disease, Graves' disease, adverse drug reactions, or chronic graft vasculopathy; allergic bronchopulmonary aspergillosis, allergic neuritis, drug allergy, radiation-induced lung injury, eosinophilic pneumonia, radiographic contrast media allergy, bronchiolitis obliterans, or interstitial pneumonia; parkinsonism-dementia complex, sporadic frontotemporal dementia, frontotemporal dementia with Parkinsonism linked to chromosome 17, frontotemporal lobar degeneration, tangle only dementia, cerebral amyloid angiopathy, cerebrovascular disorder, certain forms of frontotemporal dementia, chronic traumatic encephalopathy (CTE), Parkinson's Disease with dementia (PDD), argyrophilic grain dementia, dementia pugilistica, dementia with Lewy Bodies (DLB), or multi-infarct dementia; Creutzfeldt-Jakob disease, Huntington's disease, multifocal motor neuropathy (MMN), prion protein cerebral amyloid angiopathy, polymyositis, postencephalitic parkinsonism, subacute sclerosing panencephalitis, non-Guamanian motor neuron disease with neurofibrillary tangles, neural regeneration, and diffuse neurofibrillary tangles with calcification.

In some embodiments, the disorder is selected from: atopic dermatitis, dermatitis, dermatomyositis bullous pemphigoid, scleroderma, sclerodermatomyositis, psoriatic arthritis, pemphigus vulgaris, Discoid lupus erythematosus, cutaneous lupus, chilblain lupus erythematosus, or lupus erythematosus-lichen planus overlap syndrome; cryoglobulinemic vasculitis, mesenteric/enteric vascular disorder, peripheral vascular disorder, antineutrophil cytoplasm antibody (ANCA)-associated vasculitis (AAV), IL-2 induced vascular leakage syndrome, immune complex vasculitis, angioedema, low platelets (HELLP) syndrome, sickle cell disease, platelet refractoriness, red cell casts, or typical or infectious hemolytic uremic syndrome (tHUS); hematuria, hemorrhagic shock, drug-induced thrombocytopenia, autoimmune hemolytic anemia (AIHA), azotemia, blood vessel and/or lymph vessel inflammation, rotational atherectomy, or delayed hemolytic transfusion reaction; British type amyloid angiopathy, Buerger's disease, bullous pemphigoid, C1q nephropathy, cancer, and catastrophic antiphospholipid syndrome. In some embodiments, the disorder is autoimmune hemolytic anemia, e.g., warm autoimmune hemolytic anemia.

In another embodiment, the disorder is selected from: wet (exudative) AMD, dry (non-exudative) AMD, chorioretinal degeneration, choroidal neovascularization (CNV), choroiditis, loss of RPE function, loss of vision (including loss of visual acuity or visual field), loss of vision from AMD, retinal damage in response to light exposure, retinal degeneration, retinal detachment, retinal dysfunction, retinal neovascularization (RNV), retinopathy of prematurity, pathological myopia, or RPE degeneration; pseudophakic bullous keratopathy, symptomatic macular degeneration related disorder, optic nerve degeneration, photoreceptor degeneration, cone degeneration, loss of photoreceptor cells, pars planitis, scleritis, proliferative vitreoretinopathy, or formation of ocular drusen; chronic urticaria, Churg-Strauss syndrome, cold agglutinin disease (CAD), corticobasal degeneration (CBD), cryoglobulinemia, cyclitis, damage of the Bruch's membrane, Degos disease, diabetic angiopathy, elevated liver enzymes, endotoxemia, epidermolysis bullosa, or epidermolysis bullosa acquisita; essential mixed cryoglobulinemia, excessive blood urea nitrogen-BUN, focal segmental glomerulosclerosis, Gerstmann-Straussler-Scheinker disease, giant cell arteritis, gout, Hallervorden-Spatz disease, Hashimoto's thyroiditis, Henoch-Schonlein purpura nephritis, or abnormal urinary sediments; hepatitis, hepatitis A, hepatitis B, hepatitis C or human immunodeficiency virus (HIV), a viral infection more generally, for example selected from Flaviviridae, Retroviruses, Coronaviridae, Poxviridae, Adenoviridae, Herpesviridae, Caliciviridae, Reoviridae, Picornaviridae, Togaviridae, Orthomyxoviridae, Rhabdoviridae, or Hepadnaviridae; Neisseria meningitidis, shiga toxin E. coli-related hemolytic uremic syndrome (STEC-HUS), hemolytic uremic syndrome (HUS); Streptococcus, and poststreptococcal glomerulonephritis.

In a further embodiment, the disorder is selected from: hyperlipidemia, hypertension, hypoalbuminemia, hypovolemic shock, hypocomplementemic urticarial vasculitis syndrome, hypophosphastasis, hypovolemic shock, idiopathic pneumonia syndrome, or idiopathic pulmonary fibrosis; inclusion body myositis, intestinal ischemia, iridocyclitis, iritis, juvenile chronic arthritis, Kawasaki's disease (arteritis), or lipiduria; membranoproliferative glomerulonephritis (MPGN) I, microscopic polyangiitis, mixed cryoglobulinemia, molybdenum cofactor deficiency (MoCD) type A, pancreatitis, panniculitis, Pick's disease, polyarteritis nodosa (PAN), progressive subcortical gliosis, proteinuria, reduced glomerular filtration rate (GFR), or renovascular disorder; multiple organ failure, multiple system atrophy (MSA), myotonic dystrophy, Niemann-Pick disease type C, chronic demyelinating diseases, or progressive supranuclear palsy; spinal cord injury, spinal muscular atrophy, spondyloarthropathies, Reiter's syndrome, spontaneous fetal loss, recurrent fetal loss, pre-eclampsia, synucleinopathy, Takayasu's arteritis, post-partum thyroiditis, thyroiditis, Type I cryoglobulinemia, Type II mixed cryoglobulinemia, Type III mixed cryoglobulinemia, ulcerative colitis, uremia, urticaria, venous gas embolus (VGE), or Wegener's granulomatosis; von Hippel-Lindau disease, histoplasmosis of the eye, hard drusen, soft drusen, pigment clumping, and photoreceptor and/or retinal pigmented epithelia (RPE) loss.

In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein is useful for treating a disorder selected from autoimmune oophoritis, endometriosis, autoimmune orchitis, Ord's thyroiditis, autoimmune enteropathy, coeliac disease, Hashimoto's encephalopathy, antiphospholipid syndrome (APLS) (Hughes syndrome), aplastic anemia, autoimmune lymphoproliferative syndrome (Canale-Smith syndrome), autoimmune neutropenia, Evans syndrome, pernicious anemia, pure red cell aplasia, thrombocytopenia, adipose dolorosa (Dercum's disease), adult onset Still's disease, ankylosing spondylitis, CREST syndrome, drug-induced lupus, eosinophilic fasciitis (Shulman's syndrome), Felty syndrome, IgG4-related disease, mixed connective tissue disease (MCTD), palindromic rheumatism (Hench-Rosenberg syndrome), Parry-Romberg syndrome, Parsonage-Turner syndrome, relapsing polychondritis (Meyenburg-Altherr-Uehlinger syndrome), retroperitonial fibrosis, rheumatic fever, Schnitzler syndrome, fibromyalgia, neuromyotonia (Isaac's disease), paraneoplastic degeneration, autoimmune inner ear disease, Meniere's disease, interstitial cystitis, autoimmune pancreatitis, zika virus-related disorders, chikungunya virus-related disorders, subacute bacterial endocarditis (SBE), IgA nephropathy, IgA vasculitis, polymyalgia rheumatic, rheumatoid vasculitis, alopecia areata, autoimmune progesterone dermatitis, dermatitis herpetiformis, erythema nodosum, gestational pemphigoid, hidradenitis suppurativa, lichen sclerosus, linear IgA disease (LAD), morphea, myositis, pityriasis lichenoides et varioliformis acuta, vitiligo post-myocardial infarction syndrome (Dressler's syndrome), post-pericardiotomy syndrome, autoimmune retinopathy, Cogan syndrome, Graves opthalmopathy, ligneous conjunctivitis, Mooren's ulcer, opsoclonus myoclonus syndrome, optic neuritis, retinocochleocerebral vasculopathy (Susac's syndrome), sympathetic ophthalmia, Tolosa-Hunt syndrome, interstitial lung disease, antisynthetase syndrome, Addison's disease, autoimmune polyendocrine syndrome (APS) type I, autoimmune polyendocrine syndrome (APS) type II, autoimmune polyendocrine syndrome (APS) type III, disseminated sclerosis (multiple sclerosis, pattern II), rapidly progressing glomerulonephritis (RPGN), juvenile rheumatoid arthritis, enthesitis-related arthritis, reactive arthritis (Reiter's syndrome), autoimmune hepatitis or lupoid hepatitis, primary biliary cirrhosis (PBS), primary sclerosing cholangitis, microscopic colitis, latent lupus (undifferentiated connective tissue disease (UCTD)), acute disseminated encephalomyelitis (ADEM), acute motor axonal neuropathy, anti-(R)—N-methyl-D-aspartate receptor encephalitis, Balo concentric sclerosis (Schilders disease), Bickerstaff's encephalitis, chronic inflammatory demyelinating polyneuropathy, idiopathic inflammatory demyelinating disease, Lambert-Eaton mysathenic syndrome, Oshtoran syndrome, pediatric autoimmune neuropsychiatric disorder associated with streptococcus (PANDAS), progressive inflammatory neuropathy, restless leg syndrome, stiff person syndrome, Sydenhem syndrome, transverse myelitis, lupus vasculitis, leukocytoclastic vasculitis, Microscopic Polyangiitis, polymyositis, and ischemic-reperfusion injury of the eye.

Examples of eye disorders that may be treated according to the compositions and methods disclosed herein include amoebic keratitis, fungal keratitis, bacterial keratitis, viral keratitis, onchocercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma, and combinations thereof.

In a further embodiment, the disorder is selected from glaucoma, diabetic retinopathy, blistering cutaneous diseases (including bullous pemphigoid, pemphigus, and epidermolysis bullosa), ocular cicatricial pemphigoid, uveitis, adult macular degeneration, diabetic retinopathy, retinitis pigmentosa, macular edema, diabetic macular edema, Behcet's uveitis, multifocal choroiditis, Vogt-Koyanagi-Harada syndrome, intermediate uveitis, birdshot retinochorioditis, sympathetic ophthalmia, ocular cicatricial pemphigoid, ocular pemphigus, nonarteritic ischemic optic neuropathy, postoperative inflammation, and retinal vein occlusion, and central retinal vein occlusion (CVRO).

In some embodiments, a method for the treatment of an autoimmune blistering disease in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, a method for the treatment of bullous pemphigoid in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, the complement mediated disorder is an ophthalmic disease (e.g., early or neovascular age-related macular degeneration and geographic atrophy), an autoimmune disease (e.g., arthritis or rheumatoid arthritis), a respiratory diseases, or a cardiovascular disease. In other embodiments, the compounds of the disclosure are suitable for use in the treatment of diseases and disorders associated with fatty acid metabolism, including obesity and other metabolic disorders.

In some embodiments, a method for the treatment of geographic atrophy in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

Disorders that may be treated or prevented by an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein also include, but are not limited to: hereditary angioedema, capillary leak syndrome, hemolytic uremic syndrome (HUS), neurological disorders, Guillain-Barre Syndrome, diseases of the central nervous system and other neurodegenerative conditions, glomerulonephritis (including membrane proliferative glomerulonephritis), SLE nephritis, proliferative nephritis, liver fibrosis, tissue regeneration and neural regeneration, or Barraquer-Simons Syndrome; inflammatory effects of sepsis, systemic inflammatory response syndrome (SIRS), disorders of inappropriate or undesirable complement activation, interleukin-2 induced toxicity during IL-2 therapy, inflammatory disorders, inflammation of autoimmune diseases, systemic lupus erythematosus (SLE), lupus nephritis, arthritis, immune complex disorders and autoimmune diseases, systemic lupus, or lupus erythematosus; ischemia/reperfusion injury (I/R injury), myocardial infarction, myocarditis, post-ischemic reperfusion conditions, balloon angioplasty, atherosclerosis, post-pump syndrome in cardiopulmonary bypass or renal bypass, renal ischemia, mesenteric artery reperfusion after aortic reconstruction, antiphospholipid syndrome, autoimmune heart disease, ischemia-reperfusion injuries, obesity, or diabetes; Alzheimer's dementia, stroke, schizophrenia, traumatic brain injury, trauma, Parkinson's disease, epilepsy, transplant rejection, prevention of fetal loss, biomaterial reactions (e.g. in hemodialysis, implants), hyperacute allograft rejection, xenograft rejection, transplantation, psoriasis, burn injury, thermal injury including burns or frostbite, or crush injury; asthma, allergy, acute respiratory distress syndrome (ARDS), cystic fibrosis, adult respiratory distress syndrome, dyspnea, hemoptysis, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), bronchoconstriction, hypersensitivity pneumonitis, parasitic diseases, Goodpasture's Syndrome (anti-glomerular basement membrane nephritis), pulmonary vasculitis, Pauci-immune vasculitis, and immune complex-associated inflammation.

In some embodiments, a method for the treatment of sickle cell disease in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, a method for the treatment of immune thrombocytopenic purpura (ITP), thrombotic thrombocytopenic purpura (TTP), or idiopathic thrombocytopenic purpura (ITP) in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), Table 1) or its salt or composition as described herein. In some embodiments, a method for the treatment of immune thrombocytopenic purpura (ITP).

In some embodiments, a method for the treatment of ANCA-vasculitis in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, a method for the treatment of IgA nephropathy in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), Table 1) or its salt or composition as described herein.

In some embodiments, a method for the treatment of rapidly progressing glomerulonephritis (RPGN), in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, a method for the treatment of lupus nephritis, in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In some embodiments, a method for the treatment of hemorrhagic dengue fever, in a subject is provided that includes the administration of an effective amount of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein.

In an additional alternative embodiment, an active compound (e.g., a compound of formula (I), (I′), (II), (IIA), (III), (IIIA), (IV), (IVA), (V), (VA), (VB), (VC), (VI), (VIA), (VIB), (VIC), or (VII), Table 1) or its salt or composition as described herein is used in the treatment of an autoimmune disorder. The complement pathway enhances the ability of antibodies and phagocytic cells to clear microbes and damaged cells from the body. It is part of the innate immune system and in healthy individuals is an essential process. Inhibiting the complement pathway will decrease the body's immune system response. Therefore, it is an object of the present disclosure to treat autoimmune disorders by administering an effective does of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to a subject in need thereof.

In some embodiments, the autoimmune disorder is caused by activity of the complement system.

In some embodiments the autoimmune disorder is caused by activity of the alternative complement pathway. In some embodiments the autoimmune disorder is caused by activity of the classical complement pathway. In another embodiment the autoimmune disorder is caused by a mechanism of action that is not directly related to the complement system, such as the over-proliferation of T-lymphocytes or the over-production of cytokines.

Non-limiting examples of autoimmune disorders include: lupus, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), diabetes, multiple sclerosis, pernicious anemia, psoriasis, rheumatoid arthritis, sarcoidosis, and scleroderma.

In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein is used in the treatment of lupus. Non-limiting examples of lupus include lupus erythematosus, cutaneous lupus, discoid lupus erythematosus, chilblain lupus erythematosus, and lupus erythematosus-lichen planus overlap syndrome.

Lupus erythematosus is a general category of disease that includes both systemic and cutaneous disorders. The systemic form of the disease can have cutaneous as well as systemic manifestations. However, there are also forms of the disease that are only cutaneous without systemic involvement. For example, SLE is an inflammatory disorder of unknown etiology that occurs predominantly in women, and is characterized by articular symptoms, butterfly erythema, recurrent pleurisy, pericarditis, generalized adenopathy, splenomegaly, as well as CNS involvement and progressive renal failure. The sera of most patients (over 98%) contain antinuclear antibodies, including anti-DNA antibodies. High titers of anti-DNA antibodies are essentially specific for SLE. Conventional treatment for this disease has been the administration of corticosteroids or immunosuppressants.

There are three forms of cutaneous lupus: chronic cutaneous lupus (also known as discoid lupus erythematosus or DLE), subacute cutaneous lupus, and acute cutaneous lupus. DLE is a disfiguring chronic disorder primarily affecting the skin with sharply circumscribed macules and plaques that display erythema, follicular plugging, scales, telangiectasia, and atrophy. The condition is often precipitated by sun exposure, and the early lesions are erythematous, round scaling papules that are 5 to 10 mm in diameter and display follicular plugging. DLE lesions appear most commonly on the cheeks, nose, scalp, and ears, but they may also be generalized over the upper portion of the trunk, extensor surfaces of the extremities, and on the mucous membranes of the mouth. If left untreated, the central lesion atrophies and leaves a scar. Unlike SLE, antibodies against double-stranded DNA (e.g., DNA-binding test) are almost invariably absent in DLE.

Diabetes can refer to either type 1 or type 2 diabetes. In some embodiments an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein is provided at an effective dose to treat a patient with type 1 diabetes. In some embodiments an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein is provided at an effective dose to treat a patient with type 2 diabetes. Type 1 diabetes is an autoimmune disease. An autoimmune disease results when the body's system for fighting infection (the immune system) attacks a part of the body. In the case of diabetes type 1, the pancreas then produces little or no insulin.

In some embodiments, the complement-mediated disease or disorder comprises transplant rejection. In some embodiments, the complement-mediated disease or disorder is antibody-mediated transplant rejection.

In certain aspects, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein is used to treat a proliferative disorder, including, but not limited to, cancer. Targeted cancers suitable for administration of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt described herein include, but are not limited to, estrogen-receptor positive cancer, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, adenocarcinoma of the colon, adenocarcinoma of the rectum, central nervous system germ cell tumors, teratomas, estrogen receptor-negative breast cancer, estrogen receptor-positive breast cancer, familial testicular germ cell tumors, HER2-negative breast cancer, HER2-positive breast cancer, male breast cancer, ovarian immature teratomas, ovarian mature teratoma, ovarian monodermal and highly specialized teratomas, progesterone receptor-negative breast cancer, progesterone receptor-positive breast cancer, recurrent breast cancer, recurrent colon cancer, recurrent extragonadal germ cell tumors, recurrent extragonadal non-seminomatous germ cell tumor, recurrent extragonadal seminomas, recurrent malignant testicular germ cell tumors, recurrent melanomas, recurrent ovarian germ cell tumors, recurrent rectal cancer, stage III extragonadal non-seminomatous germ cell tumors, stage III extragonadal seminomas, stage III malignant testicular germ cell tumors, stage III ovarian germ cell tumors, stage IV breast cancers, stage IV colon cancers, stage IV extragonadal non-seminomatous germ cell tumors, stage IV extragonadal seminoma, stage IV melanomas, stage IV ovarian germ cell tumors, stage IV rectal cancers, testicular immature teratomas, testicular mature teratomas. In particular embodiments, the targeted cancers included estrogen-receptor positive, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, metastatic colorectal cancer, metastatic melanoma with CDK4 mutation or amplification, or cisplatin-refractory, unresectable germ cell tumors, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, fibrosarcoma, myxosarcoma, chondrosarcoma, osteosarcoma, chordoma, malignant fibrous histiocytoma, hemangiosarcoma, angiosarcoma, lymphangiosarcoma, Mesothelioma, leiomyosarcoma, rhabdomyosarcoma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phyllodes, salivary cancers, thymic carcinomas, bladder cancer, and Wilms tumor, a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others, T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sezary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); blastic NK-cell lymphoma; enteropathy-type T-cell lymphoma; hepatosplenic gamma-delta T-cell lymphoma; lymphoblastic Lymphoma; nasal NK/T-cell lymphomas; treatment-related T-cell lymphomas; for example lymphomas that appear after solid organ or bone marrow transplantation; T-cell prolymphocytic leukemia; T-cell large granular lymphocytic leukemia; chronic lymphoproliferative disorder of NK-cells; aggressive NK cell leukemia; systemic EBV+ T-cell lymphoproliferative disease of childhood (associated with chronic active EBV infection); hydroa vacciniforme-like lymphoma; adult T-cell leukemia/lymphoma; Enteropathy-associated T-cell lymphoma; Hepatosplenic T-cell lymphoma; or Subcutaneous panniculitis-like T-cell lymphoma.

In some embodiments, the methods described herein can be used to treat a subject, for example a human, with a lymphoma or lymphocytic or myelocytic proliferation disorder or abnormality. For example, the methods as described herein can be administered to a subject with a Hodgkin Lymphoma or a Non-Hodgkin Lymphoma. For example, the subject can have a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia, a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL, a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+DLBCL of the elderly; Primary mediastinal (thymic) large B-cell lymphoma; Primary cutaneous DLBCL, leg type; ALK+large B-cell lymphoma; plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma, a leukemia, for example, an acute or chronic leukemia of a lymphocytic or myelogenous origin, such as, but not limited to: acute lymphoblastic leukemia (ALL); Acute myelogenous leukemia (AML); chronic lymphocytic leukemia (CLL); chronic myelogenous leukemia (CML); juvenile myelomonocytic leukemia (JMML); hairy cell leukemia (HCL); acute promyelocytic leukemia (a subtype of AML); large granular lymphocytic leukemia; or adult T-cell chronic leukemia. In some embodiments, the patient has an acute myelogenous leukemia, for example an undifferentiated AML (MO); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7), small cell lung cancer, retinoblastoma, HPV positive malignancies like cervical cancer and certain head and neck cancers, MYC amplified tumors such as Burkitts' Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancers, and certain classes of testicular cancers.

In certain aspects, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt as described herein can be used to preserve or prevent damage to an organ or blood product. For example, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt described herein can be used to prevent damage to an organ, tissue, cell product, or blood product, that has been harvested for transplantation. In some embodiments, the organ is the heart, kidney, pancreas, lung, liver, or intestine. In some embodiments, the tissue is derived from the cornea, bone, tendon, muscle, heart valve, nerve, artery or vein, or the skin. In some embodiments, the blood product is whole blood, plasma, red blood cells or reticulocytes.

In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein prevents or delays the onset of at least one symptom of a complement-mediated disease or disorder in an individual. In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein reduces or eliminates at least one symptom of a complement-mediated disease or disorder in an individual. Examples of symptoms include, but are not limited to, symptoms associated with autoimmune disease, cancer, hematological disease, infectious disease, inflammatory disease, ischemia-reperfusion injury, neurodegenerative disease, neurodegenerative disorder, renal disease, transplant rejection, ocular disease, vascular disease, or a vasculitis disorder. The symptom can be a neurological symptom, for example, impaired cognitive function, memory impairment, loss of motor function, etc. The symptom can also be the activity of C1s protein in a cell, tissue, or fluid of an individual. The symptom can also be the extent of complement activation in a cell, tissue, or fluid of an individual.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual modulates complement activation in a cell, tissue, or fluid of an individual. In some embodiments, administration of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual inhibits complement activation in a cell, tissue, or fluid of an individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein, when administered in one or more doses as monotherapy or in combination therapy to an individual having a complement-mediated disease or disorder, inhibits complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the compounds described herein.

In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein reduces C3 deposition onto red blood cells; for example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein reduces deposition of C3b, iC3b, etc., onto RBCs. In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein inhibits complement-mediated red blood cell lysis.

In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein reduces C3 deposition onto platelets; for example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein reduces deposition of C3b, iC3b, etc., onto platelets.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), Table 1) or its salt or composition as described herein results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (1) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction of C3 deposition on red blood cells (e.g., a reduction of deposition of C3b, iC3b, etc., onto RBCs); and (ad) a reduction in C3 deposition on platelets (e.g., a reduction of deposition of C3b, iC3b, etc., onto platelets); and (ae) a reduction of anaphylatoxin toxin production; (af) a reduction in autoantibody mediated blister formation; (ag) a reduction in autoantibody induced pruritis; (ah) a reduction in autoantibody induced erythematosus; (ai) a reduction in autoantibody mediated skin erosion; (aj) a reduction in red blood cell destruction due to transfusion reactions; (ak) a reduction in red blood cell lysis due to alloantibodies; (al) a reduction in hemolysis due to transfusion reactions; (am) a reduction in allo-antibody mediated platelet lysis; (an) a reduction in platelet lysis due to transfusion reactions; (ao) a reduction in mast cell activation; (ap) a reduction in mast cell histamine release; (aq) a reduction in vascular permeability; (ar) a reduction in edema; (as) a reduction in complement deposition on transplant graft endothelium; (at) a reduction of anaphylatoxin generation in transplant graft endothelium; (au) a reduction in the separation of the dermal-epidermal junction; (av) a reduction in the generation of anaphylatoxins in the dermal-epidermal junction; (aw) a reduction in alloantibody mediated complement activation in transplant graft endothelium; (ax) a reduction in antibody mediated loss of the neuromuscular junction; (ay) a reduction in complement activation at the neuromuscularjunction; (az) a reduction in anaphylatoxin generation at the neuromuscular junction; (ba) a reduction in complement deposition at the neuromuscular junction; (bb) a reduction in paralysis; (bc) a reduction in numbness; (bd) increased bladder control; (be) increased bowel control; (bf a reduction in mortality associated with autoantibodies; and (bg) a reduction in morbidity associated with autoantibodies.

In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein, when administered in one or more doses to an individual having a complement-mediated disease or disorder, is effective to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; (j) oligodendrocyte loss; (k) dendritic cell infiltration; (I) neutrophil infiltration; (m) red blood cell lysis; (n) red blood cell phagocytosis; (o) platelet phagocytosis; (p) platelet lysis; (q) transplant graft rejection; I macrophage mediated phagocytosis; (s) vision loss; (t) antibody mediated complement activation; (u) autoantibody mediated complement activation; (v) demyelination; (w) eosinophilia; compared to the level or degree of the outcome in the individual before treatment with the active compound or its salt.

In some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein, when administered in one or more doses to an individual having a complement-mediated disease or disorder, is effective to achieve an improvement of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation; f) clotting; g) kidney function; and h) hematocrit (red blood cell count), compared to the level or degree of the outcome in the individual before treatment with the active compound or its salt.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual reduces complement activation in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein, when administered in one or more doses to an individual having a complement-mediated disease or disorder, reduces complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the active compound or its salt.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein improves cognitive function in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) described herein, when administered in one or more doses to an individual having a complement-mediated disease or disorder, improves cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the cognitive function in the individual before treatment with the active compound or its salt.

In some embodiments, administering an active compound (e.g., a compound of formula (I), (I′), (II), (IIA), (III), (IIA), (IV), (IVA), (V), (VA), (VB), (VC), (VI), (VIA), (VIB), (VIC), or (VII), or Table 1) or its salt or composition as described herein reduces the rate of decline in cognitive function in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt, when administered in one or more doses to an individual having a complement-mediated disease or disorder, reduces the rate of decline of cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the rate of decline in cognitive function in the individual before treatment with the active compound or its salt.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual reduces neuron loss in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt, when administered in one or more doses to an individual having a complement-mediated disease or disorder, reduces neuron loss in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to neuron loss in the individual before treatment with the active compound or its salt.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual reduces phospho-Tau levels in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt, when administered in one or more doses to an individual having a complement-mediated disease or disorder, reduces phospho-Tau in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the phospho-Tau level in the individual before treatment with the active compound or its salt.

In some embodiments, administering an active compound (e.g., a compound of formula (I), (I′), (II), (IA), (III), (IIA), (IV), (IVA), (V), (VA), (VB), (VC), (VI), (VIA), (VIB), (VIC), or (VII), or Table 1) or its salt or composition as described herein to an individual reduces glial cell activation in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt, when administered in one or more doses to an individual having a complement-mediated disease or disorder, reduces glial activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to glial cell activation in the individual before treatment with the active compound or its salt. In some embodiments, the glial cells are astrocytes or microglia.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual reduces lymphocyte infiltration in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt, when administered in one or more doses to an individual having a complement-mediated disease or disorder, reduces lymphocyte infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to lymphocyte infiltration in the individual before treatment with the active compound or its salt.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual reduces macrophage infiltration in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt, when administered in one or more doses to an individual having a complement-mediated disease or disorder, reduces macrophage infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to macrophage infiltration in the individual before treatment with the active compound or its salt.

In some embodiments, administering an active compound (e.g., a compound of (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual reduces antibody deposition in the individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt, when administered in one or more doses to an individual having a complement-mediated disease or disorder, reduces antibody deposition in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to antibody deposition in the individual before treatment with the active compound or its salt.

In some embodiments, administering an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt or composition as described herein to an individual reduces anaphylatoxin (e.g., C3a, C4a, C5a) production in an individual. For example, in some embodiments, an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt, when administered in one or more doses to an individual having a complement-mediated disease or disorder, reduces anaphylatoxin production in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of anaphylatoxin production in the individual before treatment with the active compound or its salt.

The present disclosure provides a use of an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt of the present disclosure or a pharmaceutical composition comprising an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt of the present disclosure and a pharmaceutically acceptable excipient to treat an individual having a complement-mediated disease or disorder. In some embodiments, the present disclosure provides a use of an active compound (e.g., a compound of formula (I), (I′), (II), (IIA), (III), (IIIA), (IV), (IVA), (V, (VA), (VB), (VC), (VI), (VIA), (VIB), (VIC), or (VII), or Table 1) or its salt of the present disclosure to treat an individual having a complement-mediated disease or disorder. In some embodiments, the present disclosure provides a use of a pharmaceutical composition comprising an active compound (e.g., a compound of formula (I), (I′), (II), (IIA), (III), (IIIA), (IV), (IVA), (V), (VA), (VB), (VC), (VI), (VIA), (VIB), (VIC), or (VII), or Table 1) or its salt of the present disclosure and a pharmaceutically acceptable excipient to treat an individual having a complement-mediated disease or disorder.

### EXAMPLES

The following examples are merely illustrative and should not be construed as limiting the scope of this disclosure in any way as many variations and equivalents will become apparent to those skilled in the art upon reading the present disclosure. The contents of all references, patents, and patent applications cited throughout this application are expressly incorporated herein by reference.

### Example 1. Non-Limiting Synthetic Examples of Compounds of the Present Disclosure

The below schemes are non-limiting examples of methods to make compounds of the present disclosure. The skilled artisan will recognize that there are various modifications that can be performed to make analogs or prepare compounds in otherways. Abbreviations

| | |
|---|---|
| AcOH | acetic acid |
| AcOK | potassium acetate |
| B₂pin₂ | bis(pinacolato)diboron |
| BH₃•Sme₂ | borane dimethylsulfide |
| BH₃•THF | borane tetrahydrofuran |
| BINAP | 2,2′-bis(diphenylphosphino)-1,1′-binaphthyl |
| Cu—Tc, | cuprous 2-thiophenecarboxylate |
| Tc—Cu, | |
| CuTc, or | |
| CuTC | |
| DBU | 1,8-diazabicyclo[5.4.0]undec-7-ene |
| DCE | dichloroethane |
| DCM | dichloromethane |
| DIBAL-H | diisobutylaluminium hydride |
| DIEA, DIPEA | N,N-diisopropylethylamine |
| DMBNH₂ | 2,4-dimethoxybenzylamine |
| DMF | N,N-dimethylformamide |
| DMP | Dess-Martin periodinane |
| DMSO | dimethylsulfoxide |
| EtOAc | ethyl acetate |
| EtOH | ethanol |
| Et₃N or TEA | triethylamine |
| FA | formic acid |
| HATU | 1-[bis(dimethylamino)methylene]-1H-1,2,3-triazolo[4,5- |
| | b]pyridinium3-oxide hexafluorophosphate |
| HBTU | 2-(1H-benzotriazol-1-yl)-1,1,3,3-tetramethyluronium |
| | hexafluorophosphate |
| KOAc | potassium acetate |
| LiHMDS | lithium bis(trimethylsilyl)amide |
| m-CPBA | meta-chloroperoxybenzoic acid |
| MeCN | acetonitrile |
| MeOH | methanol |
| Me₂S | dimethyl sulfide |
| MTBE | methyl ^{t}butylether |
| NaBH(OAc)₃ | sodium triacetoxyborohydride |
| NBS | N-bromo succinimide |
| n-BuLi | n-butyllithium |
| NMI | N-methylimidazole |
| NMP | N-methyl-2-pyrrolidone |
| OTf-Ag | silver trifluoromethanesulfonate |
| PCC | pyridinium chlorochromate |
| Pd(OAc)₂ | palladium acetate |
| Pd(dppf)Cl₂ | [1,1′-bis(diphenylphosphino) |
| | ferrocene]dichloropalladium(II) |
| Pd(PPh₃)₄ | tetrakis(triphenylphosphine)palladium(0) |
| Pd(PPh₃)₂Cl₂ | bis(triphenylphosphine)palladium(II) dichloride |
| Pd/C | palladium on carbon |
| PE | petroleum ether |
| PhI(OAc)₂ | (diacetoxyiodo)benzene |
| PyBOP | benzotriazol-1-yloxytripyrrolidinophosphonium |
| | hexafluorophosphate |
| T3P or T₃P | propane phosphonic acid anhydride |
| TBAF | tetra-n-butylammonium fluoride |
| TBD | 1,5,7-triazabicyclo[4.4.0]dec-5-ene |
| TBDPSCl | tert-butyl(chloro)diphenylsilane |
| TBSCl | tert-butyldimethylsilyl chloride |
| TBTU | 2-(1H-benzotriazole-1-yl)-1,1,3,3-tetramethylaminium |
| | tetrafluoroborate |
| t-BuOK | potassium tert-butoxide |
| t-BuONO | tert-butyl nitrile |
| TCFH | chloro-N,N,N′,N′-tetramethylformamidinium |
| | hexafluorophosphate |
| TFA | trifluoroacetic acid |
| THF | tetrahydrofuran |
| TMSCHN₂ | trimethylsilyldiazomethane |
| TMSCN | trimethylsilyl cyanide |
| TsOH H₂O | p-toluenesulfonic acid monohydrate |

### General Methods

All nonaqueous reactions were performed under an atmosphere of dry argon or nitrogen gas using anhydrous solvents. The progress of reactions and the purity of target compounds were determined using one of the two liquid chromatography (LC) methods A or B disclosed herein. The structure of starting materials, intermediates, and final products was confirmed by standard analytical techniques, including NMR spectroscopy and mass spectrometry.

### LC Method A

Instrument: Waters Acquity Ultra Performance LC
Column: ACQUITY UPLC BEH C18 2.1′50 mm, 1.7 mm
Column Temperature: 40° C.
Mobile Phase: Solvent A: H₂O+0.05% FA; Solvent B: CH₃CN+0.05% FA
Flow Rate: 0.8 mL/min
Gradient: 0.24 min @ 15% B, 3.5 min gradient (15-85% B), then 0.5 min @ 85% B.
Detection: UV (210-410 nm) and MS (SQ in ES+ mode)

### Scheme 1. Synthesis of N-(1-(2-(((1H-pyrrolo[3,2-c]pyridin-2-yl)methyl)amino)-2-oxoethyl)-6-oxo-2-phenyl-1,6-dihydropyrimidin-5-yl)-3-phenylpropanamide (Compound 5)

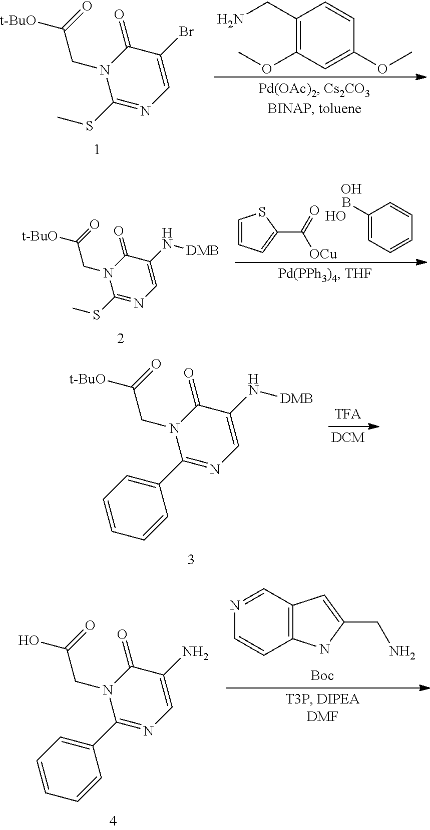

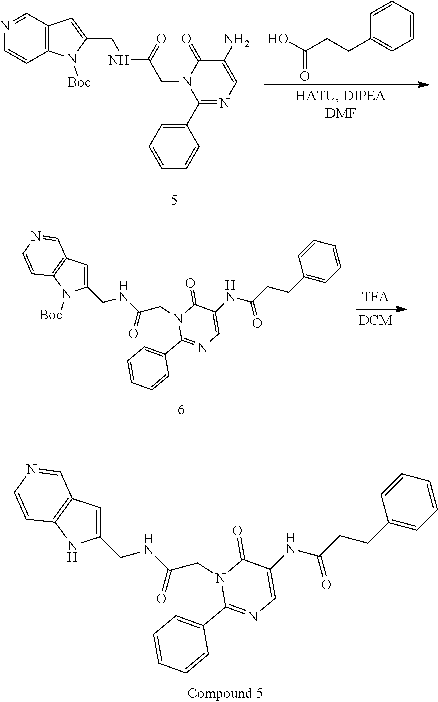 [SMILES: CC(C)(C)OC(=O)n1c(CNC(=O)Cn2c(-c3ccccc3)ncc(N)c2=O)cc2cnccc21 ; O=C(O)CCc1ccccc1 ; CC(C)(C)OC(=O)n1c(CNC(=O)Cn2c(-c3ccccc3)ncc(NC(=O)CCc3ccccc3)c2=O)cc2cnccc21 ; O=C(Cn1c(-c2ccccc2)ncc(NC(=O)CCc2ccccc2)c1=O)NCc1cc2cnccc2[nH]1]

### Step 1: Tert-butyl 2-(5-((2,4-dimethoxybenzyl)amino)-2-(methylthio)-6-oxopyrimidin-1(6H)-yl)acetate

To a mixture of tert-butyl 2-(5-bromo-2-(methylthio)-6-oxopyrimidin-1(6H)-yl)acetate (1.0 g, 2.99 mmol) and (2,4-dimethoxyphenyl)methanamine (1.0 g, 5.99 mmol) in toluene (10 mL) was added rac-BINAP (373 mg, 0.60 mmol), Cs₂CO₃ (1.95 g, 5.99 mmol) and Pd(OAc)₂ (67 mg, 0.30 mmol) under N₂ atmosphere, the mixture was degassed under N₂ atmosphere for three times and stirred at 120° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-18% EtOAc in PE) to give tert-butyl 2-(5-((2,4-dimethoxybenzyl)amino)-2-(methylthio)-6-oxopyrimidin-1(6H)-yl)acetate (1.06 g, yield 84.3%) as a yellow oil. LC/MS (ESI) m/z: 422 (M+H)⁺.

### Step 2: Tert-butyl 2-(5-((2,4-dimethoxybenzyl)amino)-6-oxo-2-phenylpyrimidin-1(6H)-yl)acetate

To a mixture of tert-butyl 2-(5-((2,4-dimethoxybenzyl)amino)-2-(methylthio)-6-oxopyrimidin-1(6H)-yl)acetate (345 mg, 0.82 mmol) and phenylboronic acid (200 mg, 1.64 mmol) in THF (6 mL) was added ((thiophene-2-carbonyl)oxy)copper (344 mg, 1.80 mmol) and Pd(PPh₃)₄ (95 mg, 0.082 mmol) under N₂ atmosphere. The mixture was degassed under N₂ atmosphere for three times and stirred at 55° C. overnight in a sealed tube. The reaction mixture was diluted with EtOAc and filtered. The filtrate was washed with saturated aq. NaHCO₃ solution and brine, dried over anhydrous Na₂SO₄, filtered, and concentrated under reduced pressure to dryness. The residue was purified by flash chromatography (silica gel, 0-24% EtOAc in PE) to give tert-butyl 2-(5-((2,4-dimethoxybenzyl)amino)-6-oxo-2-phenylpyrimidin-1(6H)-yl) acetate (310 mg, yield 83.9%) as a yellow oil. LC/MS (ESI) m/z: 452 (M+H)⁺.

### Step 3: 2-(5-Amino-6-oxo-2-phenylpyrimidin-1(6H)-yl)acetic acid

To a solution of tert-butyl 2-(5-((2,4-dimethoxybenzyl)amino)-6-oxo-2-phenylpyrimidin-1(6H)-yl)acetate (180 mg, 0.40 mmol) in DCM (5 mL) was added TFA (2 mL) and the reaction mixture was stirred at room temperature for 3 hours. The mixture was concentrated under reduced pressure to dryness to give 2-(5-amino-6-oxo-2-phenylpyrimidin-1(6H)-yl)acetic acid (97 mg, crude) as a yellow solid, which was used directly in the next step without further purification. LC/MS (ESI) m/z: 246 (M+H)⁺.

### Step 4: Tert-butyl 2-((2-(5-amino-6-oxo-2-phenylpyrimidin-1(6H)-yl)acetamido) methyl)-1H-pyrrolo[3,2-c]pyridine-1-carboxylate

To a mixture of 2-(5-amino-6-oxo-2-phenylpyrimidin-1(6H)-yl)acetic acid (80 mg, 0.33 mmol) and tert-butyl 2-(aminomethyl)-1H-pyrrolo[3,2-c]pyridine-1-carboxylate (80 mg, 0.33 mmol) in DMF (3 mL) was added DIPEA (251 mg, 1.94 mmol) and T₃P (372 mg, 0.58 mmol, 50% wt. in EtOAc) under N₂ atmosphere and the mixture was stirred at 35° C. overnight. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-5% MeOH in DCM) to give tert-butyl 2-((2-(5-amino-6-oxo-2-phenylpyrimidin-1(6H)-yl)acetamido)methyl)-1H-pyrrolo[3,2-c]pyridine-1-carboxylate (80 mg, yield 51.7%) as a light-yellow oil. LC/MS (ESI) m/z: 475 (M+H)⁺.

### Step 5: Tert-butyl 2-((2-(6-oxo-2-phenyl-5-(3-phenylpropanamido)pyrimidin-1(6H)-yl)acetamido)methyl)-1H-pyrrolo[3,2-c]pyridine-1-carboxylate

To a mixture of 3-phenylpropanoic acid (16 mg, 0.11 mmol) and HATU (48 mg, 0.13 mmol) in DMF (3 mL) was added DIPEA (40 mg, 0.31 mmol) under N₂ atmosphere and the mixture was stirred at room temperature for 30 minutes. Tert-butyl 2-((2-(5-amino-6-oxo-2-phenylpyrimidin-1(6H)-yl)acetamido)methyl)-1H-pyrrolo[3,2-c]pyridine-1-carboxylate (50 mg, 0.11 mmol) was added to the stirring mixture and the resulting mixture was stirred at 35° C. overnight. The mixture was diluted with EtOAc, washed with water and brine, dried over anhydrous Na₂SO₄, filtered, and concentrated under reduced pressure to dryness. The residue was purified by prep-TLC (DCM:MeOH=10:1) to give tert-butyl 2-((2-(6-oxo-2-phenyl-5-(3-phenylpropanamido)pyrimidin-1(6H)-yl)acetamido)methyl)-1H-pyrrolo[3,2-c]pyridine-1-carboxylate (30 mg, yield 57.8%) as a yellow oil. LC/MS (ESI) m/z: 607 (M+H)⁺.

### Step 6: N-(1-(2-(((1H-pyrrolo[3,2-c]pyridin-2-yl)methyl)amino)-2-oxoethyl)-6-oxo-2-phenyl-1,6-dihydropyrimidin-5-yl)-3-phenylpropanamide (Compound 5)

To a solution of tert-butyl 2-((2-(6-oxo-2-phenyl-5-(3-phenylpropanamido)pyrimidin-1(6H)-yl)acetamido)methyl)-1H-pyrrolo[3,2-c]pyridine-1-carboxylate (30 mg, 0.050 mmol) in DCM (1.5 mL) was added TFA (1.5 mL) and the reaction mixture was stirred at room temperature for 3 hours. The mixture was concentrated under reduced pressure to dryness. The residue was purified by prep-HPLC to give Compound 5 (6.7 mg, yield 26.8%) as a white solid. ¹H NMR (400 MHz, CD₃OD) δ 8.92 (d, J=3.3 Hz, 2H), 8.24 (d, J=6.4 Hz, 1H), 7.72 (d, J=6.2 Hz, 1H), 7.56-7.50 (m, 3H), 7.47-7.42 (m, 2H), 7.26 (d, J=4.6 Hz, 4H), 7.17 (dd, J=9.0, 4.8 Hz, 1H), 6.74 (s, 1H), 4.67 (s, 2H), 4.59 (s, 2H), 3.01 (t, J=7.6 Hz, 2H), 2.81 (t, J=7.6 Hz, 2H). LC/MS (ESI) m/z: 507 (M+H)⁺. RT (Method A): 1.14 min.

The following compounds were prepared based on Steps 5-6 in Scheme 1:

| # | Reactant A | Reactant B | Characterization Data |
|---|---|---|---|
| 4 | 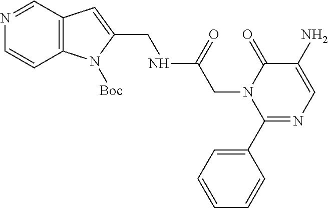 [SMILES: CC(C)(C)OC(=O)n1c(CNC(=O)Cn2c(-c3ccccc3)ncc(N)c2=O)cc2cnccc21] | 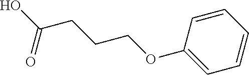 [SMILES: O=C(O)CCCOc1ccccc1] | ¹H NMR (400 MHz, CD₃OD) δ 8.94 (s, 1H), 8.85 (s, 1H), 8.20 (d, J = 5.9 Hz, 1H), 7.61 (d, J = 6.2 Hz, 1H), 7.52 (dd, J = 8.8, 7.2 Hz, 3H), 7.44 (t, J = 7.3 Hz, 2H), 7.23 (dd, J = 8.8, 7.3 Hz, 2H), 6.89 (dd, J = 11.2, 7.6 Hz, 3H), 6.66 (s, 1H), 4.67 (s, 2H), 4.58 (s, 2H), 4.06 (t, J = 6.1 Hz, 2H), 2.70 (t, J = 7.3 Hz, 2H), 2.16 (p, J = 6.7 Hz, 2H). LC/MS (ESI) m/z: 537 (M + H)⁺. RT (Method A): 1.25 min. |
| 24ª | 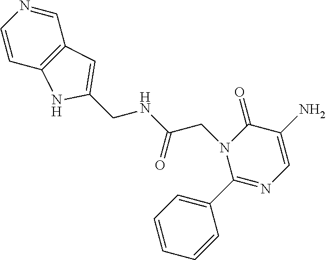 [SMILES: Nc1cnc(-c2ccccc2)n(CC(=O)NCc2cc3cnccc3[nH]2)c1=O] | 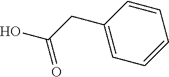 | ¹H NMR (400 MHz, CD₃OD) δ 8.89 (d, J = 18.8 Hz, 2H), 8.47 (td, J = 5.4, 3.7 Hz, 1H), 8.21 (d, J = 6.3 Hz, 1H), 7.62 (d, J = 6.2 Hz, 1H), 7.54-7.50 (m, 3H), 7.46-7.41 (m, 2H), 7.39-7.33 (m, 4H), 7.29 (ddd, J = 6.5, 3.9, 1.9 Hz, 1H), 6.66 (s, 1H), 4.66 (s, 2H), 4.57 (s, 2H), 3.83 (s, 2H). LC/MS (ESI) m/z: 493 (M + H)⁺. RT (Method A): 0.96 min. |
| 38^{a} | 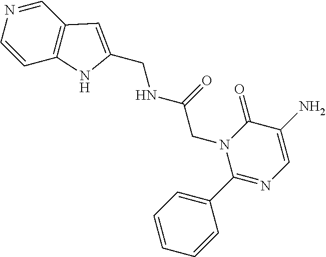 [SMILES: Nc1cnc(-c2ccccc2)n(CC(=O)NCc2cc3cnccc3[nH]2)c1=O] | 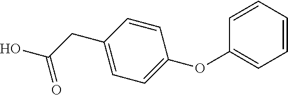 [SMILES: O=C(O)Cc1ccc(Oc2ccccc2)cc1] | ¹H NMR (400 MHz, CD₃OD) δ 8.92 (s, 1H), 8.87 (s, 1H), 8.21 (d, J = 6.3 Hz, 1H), 7.64 (dd, J = 6.2, 0.6 Hz, 1H), 7.54-7.50 (m, 3H), 7.46-7.42 (m, 2H), 7.38-7.31 (m, 4H), 7.09 (t, J = 7.4 Hz, 1H), 6.98 (d, J = 8.5 Hz, 4H), 6.69 (s, 1H), 4.67 (s, 2H), 4.59 (s, 2H), 3.82 (s, 2H). LC/MS (ESI) m/z: 585 (M + H)⁺. RT (Method A): 1.54 min. |
| 78ª | 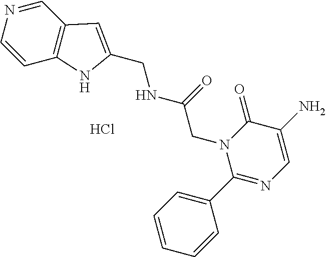 [SMILES: Nc1cnc(-c2ccccc2)n(CC(=O)NCc2cc3cnccc3[nH]2)c1=O ; Cl] | 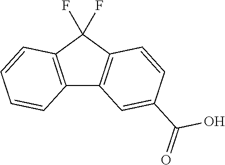 [SMILES: O=C(O)c1ccc2c(c1)-c1ccccc1C2(F)F] | ¹H NMR (400 MHz, CD₃OD) δ 9.07 (s, 1H), 8.71 (s, 1H), 8.31 (s, 1H), 8.10 (d, J = 5.8 Hz, 1H), 8.00 (dd, J = 7.8, 1.4 Hz, 1H), 7.85 (d, J = 7.7 Hz, 1H), 7.79 (d, J = 7.9 Hz, 1H), 7.67 (d, J = 7.0 Hz, 1H), 7.62 - 7.52 (m, 4H), 7.46 (d, J = 7.5 Hz, 3H), 7.38 (d, J = 5.8 Hz, 1H), 6.49 (s, 1H), 4.73 (s, 2H), 4.56 (s, 2H). LC/MS (ESI) m/z: 603 (M + H)⁺. RT (Method A): 1.68 min. |
| 90^{a} | 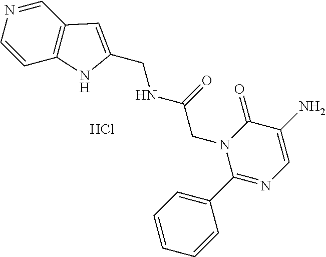 [SMILES: Nc1cnc(-c2ccccc2)n(CC(=O)NCc2cc3cnccc3[nH]2)c1=O ; Cl] | 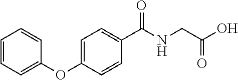 [SMILES: O=C(O)CNC(=O)c1ccc(Oc2ccccc2)cc1] | ¹H NMR (400 MHz, CD₃OD) δ 9.02 (s, 1H), 8.95 (s, 1H), 8.29 (d, J = 6.7 Hz, 1H), 7.92-7.86 (m, 3H), 7.54 (t, J = 6.9 Hz, 3H), 7.47-7.39 (m, 4H), 7.20 (t, J = 7.5 Hz, 1H), 7.07-7.00 (m, 4H), 6.87 (s, 1H), 4.69 (s, 2H), 4.62 (s, 2H), 4.27 (s, 2H). LC/MS (ESI) m/z: 628 (M + H)⁺. RT (Method A): 1.44 min. |
| 98ª | 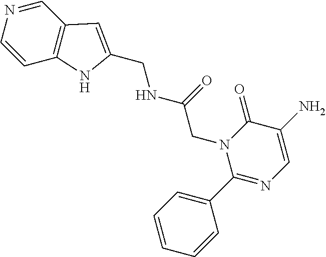 [SMILES: Nc1cnc(-c2ccccc2)n(CC(=O)NCc2cc3cnccc3[nH]2)c1=O] | 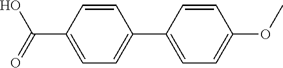 | ¹H NMR (400 MHz, CD₃OD) δ 8.11 (d, J = 5.7 Hz, 1H), 8.05 (d, J = 8.3 Hz, 2H), 7.70 (d, J = 8.3 Hz, 2H), 7.63 (d, J = 8.7 Hz, 2H), 7.47 (t, J = 6.6 Hz, 4H), 7.44-7.38 (m, 4H), 7.03 (d, J = 8.7 Hz, 2H), 6.48 (s, 1H), 4.62 (s, 2H), 4.55 (s, 2H), 3.85 (s, 3H). LC/MS (ESI) m/z: 585 (M + H)⁺. RT (Method A): 1.29 min. |
| 99^{a} | 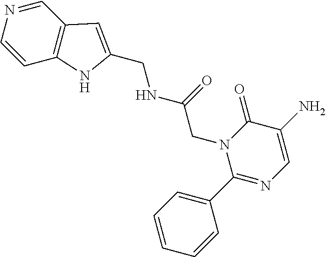 [SMILES: Nc1cnc(-c2ccccc2)n(CC(=O)NCc2cc3cnccc3[nH]2)c1=O] | 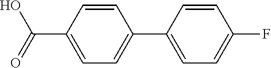 [SMILES: O=C(O)c1ccc(-c2ccc(F)cc2)cc1] | ¹H NMR (400 MHz, CD₃OD) δ 8.70 (s, 1H), 8.10 (m, 3H), 7.70 (t, J = 7.1 Hz, 4H), 7.49-7.43 (m, 4H), 7.40 (d, J = 5.6 Hz, 3H), 7.21 (m, 3H), 6.47 (s, 1H), 4.62 (s, 2H), 4.55 (s, 2H). LC/MS (ESI) m/z: 573 (M + H)⁺. RT (Method A): 1.52 min. |
| 100^{a} | 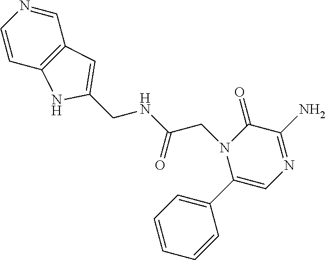 [SMILES: Nc1ncc(-c2ccccc2)n(CC(=O)NCc2cc3cnccc3[nH]2)c1=O] | 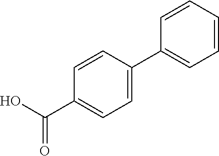 [SMILES: O=C(O)c1ccc(-c2ccccc2)cc1] | ¹H NMR (400 MHz, CD₃OD) δ 8.71 (s, 1H), 8.14-8.07 (m, 3H), 7.96 (d, J = 8.3 Hz, 1H), 7.74 (d, J = 8.2 Hz, 2H), 7.68 (d, J= 7.5 Hz, 3H), 7.47 (m, 3H), 7.40-7.38 (m, 2H), 7.35 (s, 2H), 6.66 (s, 1H), 6.47 (s, 1H), 4.53 (d, J = 3.0 Hz, 4H). LC/MS (ESI) m/z: 555 (M + H)⁺. RT (Method A): 1.47 min. |
| 101^{a} | 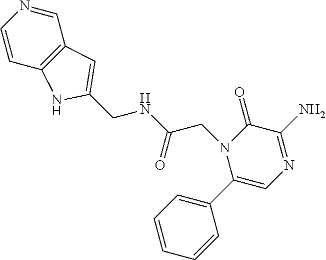 [SMILES: Nc1ncc(-c2ccccc2)n(CC(=O)NCc2cc3cnccc3[nH]2)c1=O] | 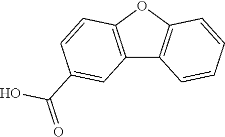 [SMILES: O=C(O)c1ccc2oc3ccccc3c2c1] | ¹H NMR (400 MHz, CD₃OD) δ 8.74 (d, J = 1.5 Hz, 1H), 8.19 (m, 1H), 8.13-8.06 (m, 3H), 7.65 (m, 2H), 7.55 (s, 1H), 7.41 (m, 3H), 7.34 (m, 4H), 6.66 (s, 1H), 6.47 (s, 1H), 4.53 (d, J = 3.0 Hz, 4H). LC/MS (ESI) m/z: 569 (M + H)⁺. RT (Method A): 1.47 min. |

^{a}Step 5 only.

### Scheme 2. Synthesis of N-(1-(2-(((1H-pyrrolo[3,2-c]pyridin-2-yl)methyl)amino)-2-oxoethyl)-6-oxo-2-phenyl-1,6-dihydropyrimidin-5-yl)-4-phenoxybenzamide (Compound 8)

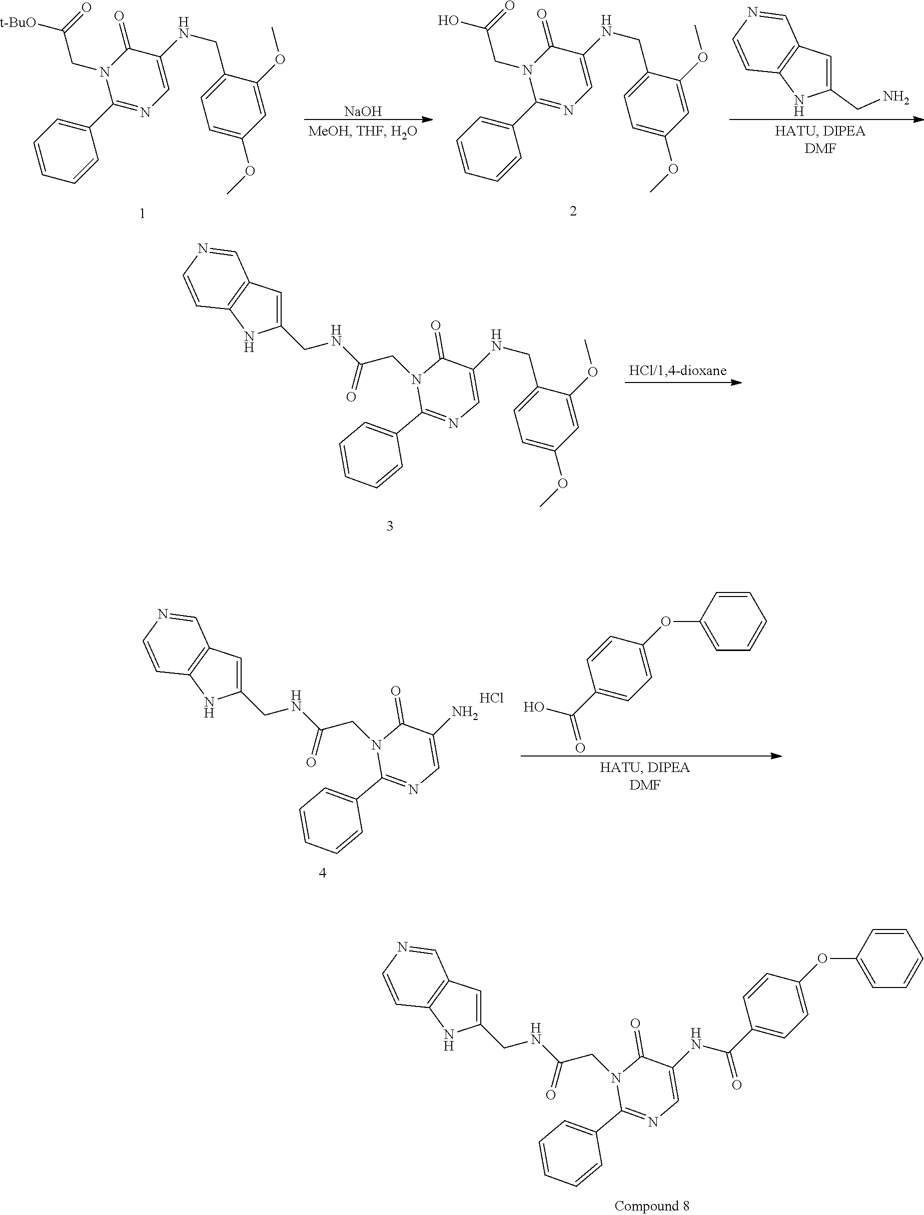

### Step 1: 2-(5-((2,4-Dimethoxybenzyl)amino)-6-oxo-2-phenylpyrimidin-1(6H)-yl)acetic acid

To a solution of tert-butyl 2-(5-((2,4-dimethoxybenzyl)amino)-6-oxo-2-phenylpyrimidin-1(6H)-yl)acetate (3.9 g, 8.6 mmol) in MeOH/H₂O/THF (40 mL, 2/1/1) was added NaOH (1.3 g, 34.4 mmol) under N₂ atmosphere and the reaction mixture was stirred at 50° C. for 2 hours. The mixture was acidified with 1N aq. HCl to pH˜3 and extracted with EtOAc twice. The combined organic layers were dried over anhydrous Na₂SO₄, filtered, and concentrated under reduced pressure to give 2-(5-((2,4-dimethoxybenzyl)amino)-6-oxo-2-phenylpyrimidin-1(6H)-yl)acetic acid (2.8 g, yield 82.4%) as a yellow solid, which was used directly in the next step without further purification. LC/MS (ESI) m/z: 396 (M+H)⁺.

### Step 2: N-((1H-pyrrolo[3,2-c]pyridin-2-yl)methyl)-2-(5-((2,4-dimethoxybenzyl) amino)-6-oxo-2-phenylpyrimidin-1(6H)-yl)acetamide

To a mixture of 2-(5-((2,4-dimethoxybenzyl)amino)-6-oxo-2-phenylpyrimidin-1(6H)-yl)acetic acid (2.0 g, 4.8 mmol) and (1H-pyrrolo[3,2-c]pyridin-2-yl)methanamine (705 mg, 4.8 mmol) in DMF (20 mL) was added DIPEA (2.8 g, 21.5 mmol) and HATU (1.8 g, 4.7 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) and to give N-((1H-pyrrolo[3,2-c]pyridin-2-yl)methyl)-2-(5-((2,4-dimethoxybenzyl)amino)-6-oxo-2-phenylpyrimidin-1(6H)-yl)acetamide (2.3 g, yield 91.7%) as a yellow solid. LC/MS (ESI) m/z: 524 (M+H)⁺.

### Step 3: N-((1H-pyrrolo[3,2-c]pyridin-2-yl)methyl)-2-(5-amino-6-oxo-2-phenylpyrimidin-1(6H)-yl)acetamide hydrochloride

A solution of N-((1H-pyrrolo[3,2-c]pyridin-2-yl)methyl)-2-(5-((2,4-dimethoxybenzyl) amino)-6-oxo-2-phenylpyrimidin-1(6H)-yl)acetamide (2.3 g, 4.4 mmol) in HCl/1,4-dioxane (20 mL, 4M) was stirred under N₂ atmosphere at room temperature for 2 hours. The reaction mixture was concentrated to dryness under reduced pressure to give N-((1H-pyrrolo[3,2-c]pyridin-2-yl)methyl)-2-(5-amino-6-oxo-2-phenylpyrimidin-1(6H)-yl)acetamide hydrochloride (1.6 g, yield 97.7%) as a brown solid, which was used directly in the next step without further purification. LC/MS (ESI) m/z: 375 (M+H)⁺.

### Step 4: N-(1-(2-(((1H-pyrrolo[3,2-c]pyridin-2-yl)methyl)amino)-2-oxoethyl)-6-oxo-2-phenyl-1,6-dihydropyrimidin-5-yl)-4-phenoxybenzamide (Compound 8)

To a mixture of N-((1H-pyrrolo[3,2-c]pyridin-2-yl)methyl)-2-(5-amino-6-oxo-2-phenylpyrimidin-1(6H)-yl)acetamide hydrochloride (30 mg, 0.080 mmol) and 4-phenoxybenzoic acid (17 mg, 0.080 mmol) in DMF (0.8 mL) was added DIPEA (41 mg, 0.032 mmol) and HATU (33 mg, 0.090 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (DCM:MeOH=8:1) and further purified by prep-HPLC to give Compound 8 (0.8 mg, yield 1.8%) as a white solid. ¹H-NMR (400 MHz, CD₃OD) δ 9.02 (s, 1H), 8.90 (s, 1H), 8.44 (s, 1H), 8.21 (d, J=6.1 Hz, 1H), 8.00-7.97 (m, 2H), 7.66 (d, J=6.2 Hz, 1H), 7.56 (dd, J=7.6, 6.1 Hz, 3H), 7.48-7.42 (m, 4H), 7.23 (t, J=7.4 Hz, 1H), 7.12-7.08 (m, 4H), 6.71 (s, 1H), 4.60 (s, 2H), 4.58 (s, 2H). LC/MS (ESI) m/z: 571 (M+H)⁺. RT (Method A): 1.50 min.

### Scheme 3. Synthesis of (S)—N-(1-(1H-pyrrolo[3,2-c]pyridin-2-yl)ethyl)-2-(6-oxo-2-phenyl-5-((3-phenylpropyl)amino)pyrimidin-1(6H)-yl)acetamide (Compound 9)

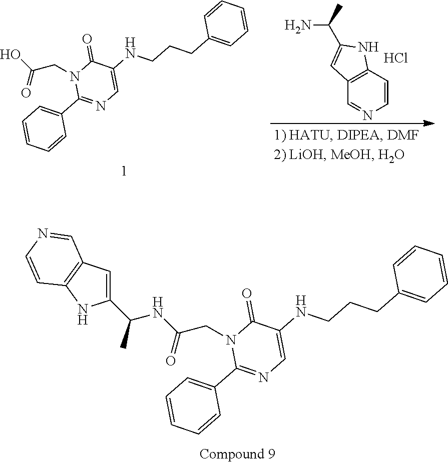 [SMILES: O=C(O)Cn1c(-c2ccccc2)ncc(NCCCc2ccccc2)c1=O ; C[C@H](N)c1cc2cnccc2[nH]1 ; Cl ; C[C@H](NC(=O)Cn1c(-c2ccccc2)ncc(NCCCc2ccccc2)c1=O)c1cc2cnccc2[nH]1]

To a mixture of (S)-1-(1H-pyrrolo[3,2-c]pyridin-2-yl)ethan-1-amine hydrochloride (25 mg, 0.083 mmol) and 2-(6-oxo-2-phenyl-5-((3-phenylpropyl)amino)pyrimidin-1(6H)-yl)acetic acid (25 mg, 0.069 mmol) in DMF (1 mL) was added DIPEA (36 mg, 0.27 mmol) and HATU (31 mg, 0.090 mmol) under N₂ atmosphere and the reaction mixture was stirred at room temperature for 1 hours. To the mixture was added LiOH (4.8 mg, 0.20 mmol), MeOH (0.5 mL) and water (0.1 mL) and the mixture was stirred at room temperature for 6 hours. The mixture was diluted with EtOAc and washed with saturated aq. NaHCO₃ solution and brine, dried over anhydrous Na₂SO₄, filtered, and concentrated under reduced pressure to dryness. The residue was purified by prep-TLC (DCM:MeOH=10:1) and further purified by prep-HPLC to give Compound 9 (5.3 mg, yield 15.2%) as a white solid. ¹H NMR (400 MHz, CD₃OD) δ 8.73 (s, 1H), 8.12 (d, J=5.9 Hz, 1H), 7.47 (d, J=6.8 Hz, 3H), 7.44-7.38 (m, 3H), 7.23 (t, J=7.9 Hz, 4H), 7.16 (t, J=7.2 Hz, 1H), 7.07 (s, 1H), 6.50 (s, 1H), 5.24 (q, J=6.8 Hz, 1H), 4.60 (t, J=13.5 Hz, 2H), 3.17 (t, J=7.0 Hz, 2H), 2.74 (t, J=7.6 Hz, 2H), 2.02-1.94 (m, 2H), 1.55 (d, J=6.9 Hz, 3H). LC/MS (ESI) m/z: 507 (M+H)⁺. RT (Method A): 1.41 min.

The following compounds were prepared based on Scheme 3:

| # | Reactant A | Reactant B | Characterization Data |
|---|---|---|---|
| 10^{a} | 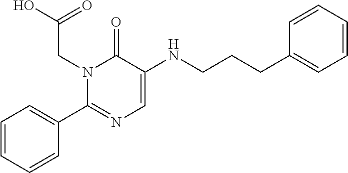 [SMILES: O=C(O)Cn1c(-c2ccccc2)ncc(NCCCc2ccccc2)c1=O] | 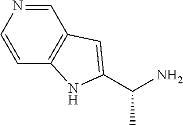 | ¹H NMR (400 MHz, CD₃OD) δ 8.71 (s, 1H), 8.10 (s, 1H), 7.47 (s, 3H), 7.41 (s, 3H), 7.26 (d, J = 6.6 Hz, 2H), 7.24 (s, 2H), 7.17 (s, 1H), 7.08 (s, 1H), 6.48 (s, 1H), 5.23 (s, 1H), 4.63 (d, J = 8.9 Hz, 2H), 3.18 (s, 3H), 2.75 (s, 2H), 2.00 (s, 2H), 1.56 (d, J = 6.9 Hz, 4H),. LC/MS (ESI) m/z: 507 (M + H)⁺. RT (Method A): 1.42 min. |
| 12 | 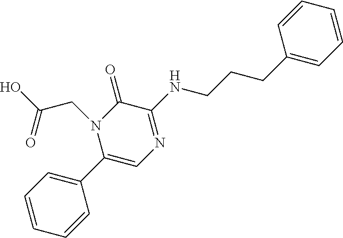 [SMILES: O=C(O)Cn1c(-c2ccccc2)cnc(NCCCc2ccccc2)c1=O] | 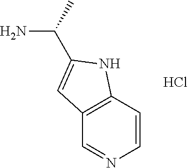 | ¹H NMR (400 MHz, CD₃OD) δ 8.70 (s, 1H), 8.10 (d, J = 5.9 Hz, 1H), 7.42- 7.33 (m, 6H), 7.23 (dd, J = 13.4, 7.2 Hz, 4H), 7.15 (t, J = 7.1 Hz, 1H), 6.71 (s, 1H), 6.45 (s, 1H), 5.25-5.20 (m, 1H), 4.56 (d, J = 16.2 Hz, 1H), 4.49 (d, J = 16.1 Hz, 1H), 3.44 (t, J = 7.0 Hz, 2H), 2.75 - 2.69 (m, 2H), 1.97 (dd, J = 15.0, 7.5 Hz, 2H), 1.54 (d, J = 6.9 Hz, 3H). LC/MS (ESI) m/z: 507 (M + H)⁺. RT (Method A): 1.51 min. |
| 13 | 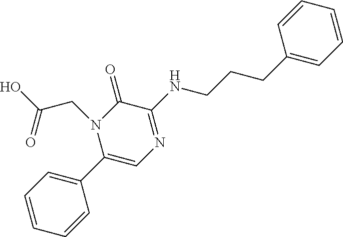 [SMILES: O=C(O)Cn1c(-c2ccccc2)cnc(NCCCc2ccccc2)c1=O] | 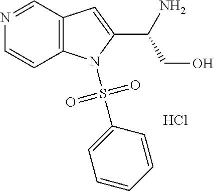 [SMILES: N[C@H](CO)c1cc2cnccc2n1S(=O)(=O)c1ccccc1 ; Cl] | ¹H NMR (400 MHz, CD₃OD) δ 8.71 (s, 1H), 8.10 (d, J = 5.8 Hz, 1H), 7.42-7.33 (m, 6H), 7.27-7.19 (m, 4H), 7.15 (d, J = 6.8 Hz, 1H), 6.71 (s, 1H), 6.49 (s, 1H), 5.22 (t, J = 5.6 Hz, 1H), 4.56 (q, J = 15.9 Hz, 2H), 3.88 (dt, J = 17.3, 11.1 Hz, 2H), 3.44 (t, J = 6.9 Hz, 2H), 2.71 (t, J = 7.7 Hz, 2H), 2.02-1.94 (m, 2H). |
| 14^{b} | 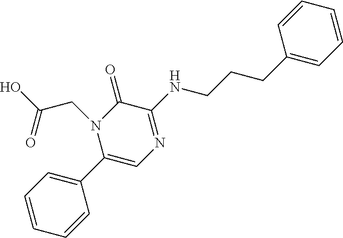 [SMILES: O=C(O)Cn1c(-c2ccccc2)cnc(NCCCc2ccccc2)c1=O] | 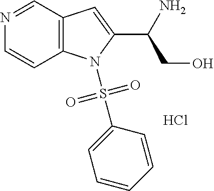 [SMILES: N[C@@H](CO)c1cc2cnccc2n1S(=O)(=O)c1ccccc1 ; Cl] | ¹H NMR (400 MHz, CD₃OD) δ 8.71 (s, 1H), 8.10 (d, J = 5.8 Hz, 1H), 7.42- 7.33 (m, 6H), 7.27-7.20 (m, 4H), 7.15 (d, J = 7.0 Hz, 1H), 6.71 (s, 1H), 6.49 (s, 1H), 5.22 (s, 1H), 4.60 (d, J = 15.8 Hz, 1H), 4.53 (d, J = 16.2 Hz, 1H), 3.88 (dt, J = 17.2, 11.3 Hz, 2H), 3.44 (t, J = 6.9 Hz, 2H), 2.72 (t, J = 7.6 Hz, 2H), 2.02-1.94 (m, 2H). LC/MS (ESI) m/z: 523 (M + H)⁺. RT (Method A): 1.38 min. |
| 15^{b} | 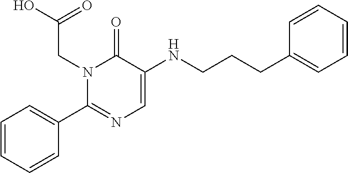 [SMILES: O=C(O)Cn1c(-c2ccccc2)ncc(NCCCc2ccccc2)c1=O] | 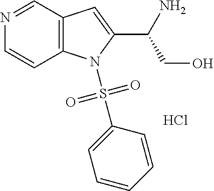 [SMILES: N[C@H](CO)c1cc2cnccc2n1S(=O)(=O)c1ccccc1 ; Cl] | ¹H NMR (400 MHz, CD₃OD) δ 8.71 (s, 1H), 8.10 (d, J = 6.0 Hz, 1H), 7.47 (d, J = 7.4 Hz, 3H), 7.40 (d, J = 6.5 Hz, 3H), 7.30-7.21 (m, 4H), 7.17 (d, J = 6.5 Hz, 1H), 7.08 (s, 1H), 6.51 (s, 1H), 5.23 (s, 1H), 4.67 (d, J = 11.7 Hz, 2H), 3.94-3.82 (m, 2H), 3.17 (t, J = 6.7 Hz, 2H), 2.74 (t, J = 7.2 Hz, 2H), 2.03-1.96 (m, 2H). LC/MS (ESI) m/z: 523 (M + H)⁺. RT (Method A): 1.30 min. |
| 16^{b} | 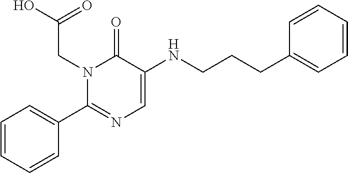 [SMILES: O=C(O)Cn1c(-c2ccccc2)ncc(NCCCc2ccccc2)c1=O] | 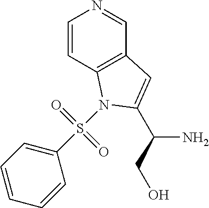 [SMILES: N[C@@H](CO)c1cc2cnccc2n1S(=O)(=O)c1ccccc1] | ¹H NMR (400 MHz, CD₃OD) δ 8.91 (s, 1H), 8.48 (s, 2H), 8.22 (s, 1H), 7.70 (s, 1H), 7.48 (s, 3H), 7.42 (d, J = 7.1 Hz, 2H), 7.26( d, J = 6.8 Hz, 2H), 7.22 (s, 1H), 7.16 (s, 1H), 7.08 (s, 1H), 6.76 (s, 1H), 5.25 (s, 1H), 4.69 (s, 3H), 3.91 (s, 3H), 3.17 (s, 3H), 2.74 (t, J = 7.1 Hz, 3H), 1.99 (d, J = 6.8 Hz, 3H). LC/MS (ESI) (m/z): 523 (M + H)⁺. RT (Method A): 1.29 min. |
| 17^{a,c} | 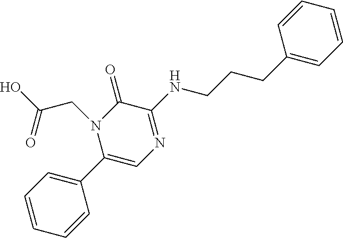 [SMILES: O=C(O)Cn1c(-c2ccccc2)cnc(NCCCc2ccccc2)c1=O] | 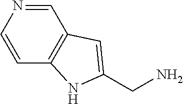 [SMILES: NCc1cc2cnccc2[nH]1] | ¹H NMR (400 MHz, CD₃OD) δ 8.89 (s, 1H), 8.22 (d, J = 6.3 Hz, 1H), 7.68 (d, J = 6.3 Hz, 1H), 7.42-7.33 (m, 5H), 7.23 (dt, J = 14.7, 7.2 Hz, 4H), 7.15 (t, J = 7.1 Hz, 1H), 6.71 (d, J = 8.1 Hz, 2H), 4.55 (d, J = 16.6 Hz, 4H), 3.44 (t, J = 7.1 Hz, 2H), 2.74-2.68 (m, 2H), 2.02- 1.93 (m, 2H). LC/MS (ESI) m/z: 493 (M + H)⁺. RT (Method A): 1.44 min. |
| 47 | 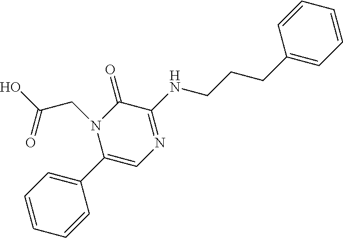 [SMILES: O=C(O)Cn1c(-c2ccccc2)cnc(NCCCc2ccccc2)c1=O] | 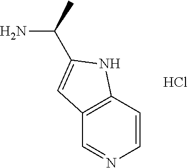 | ¹H NMR (400 MHz, CD₃OD) δ 8.78 (s, 1H), 8.52 (s, 1H), 8.14 (d, J = 6.0 Hz, 1H), 7.51 (d, J = 5.9 Hz, 1H), 7.44-7.32 (m, 5H), 7.29-7.18 (m, 4H), 7.15 (t, J = 6.6 Hz, 1H), 6.71 (s, 1H), 6.55 (s, 1H), 5.24 (q, J = 6.8 Hz, 1H), 4.52 (q, J = 16.3 Hz, 2H), 3.44 (t, J = 7.0 Hz, 2H), 2.72 (t, J = 7.7 Hz, 2H), 2.01-1.94 (m, 2H), 1.55 (d, J = 7.0 Hz, 3H). LC/MS (ESI) m/z: 507 (M + H)⁺. RT (Method A): 1.60 min. |
| 239^{a} | 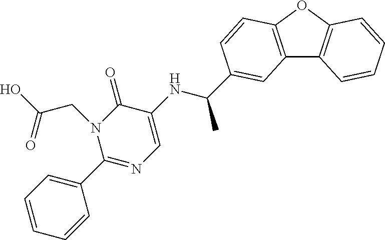 | 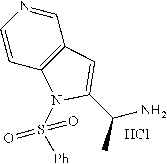 | ¹H NMR (400 MHz, DMSO-d₆) δ 11.54 (s, 1H), 8.82 (s, 1H), 8.63 (d, J = 7.9 Hz, 1H), 8.22 (d, J = 1.2 Hz, 1H), 8.16 (d, J = 5.8 Hz, 1H), 8.14 (s, 1H), 8.09 (d, J = 7.1 Hz, 1H), 7.70-7.65 (m, 2H), 7.63-7.60 (m, 1H), 7.53-7.49 (m, 1H), 7.42-7.38 (m, 3H), 7.37-7.35 (m, 2H), 7.34-7.31 (m, 1H), 6.98 (d, J = 9.1 Hz, 1H), 6.38 (d, J = 14.5 Hz, 1H), 6.00-5.89 (m, 1H), 5.13-5.06 (m, 1H), 4.70-4.64 (m, 1H), 4.57-4.44 (m, 2H), 1.61 (d, J = 6.7 Hz, 3H), 1.43 (d, J = 6.9 Hz, 3H). LC/MS (ESI) m/z: 583 (M + H)⁺. RT (Method A): 1.78 min. |
| 294 | 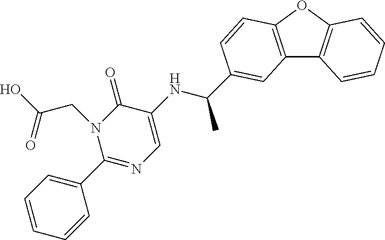 | 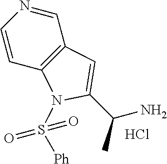 | LC/MS (ESI) m/z: 583 (M + H)⁺. RT (Method A): 1.78 min. |

^{a}Step 1 only.
^{b}Intermediate formed from the coupling reaction was isolated prior to deprotection.
^{c}HBTU was used in place of HATU.

### Scheme 4. Synthesis of N-((1H-pyrrolo[3,2-c]pyridin-2-yl)methyl)-2-(6-oxo-5-((4-phenoxybutyl)amino)-2-phenylpyrimidin-1(6H)-yl)acetamide (Compound 11)

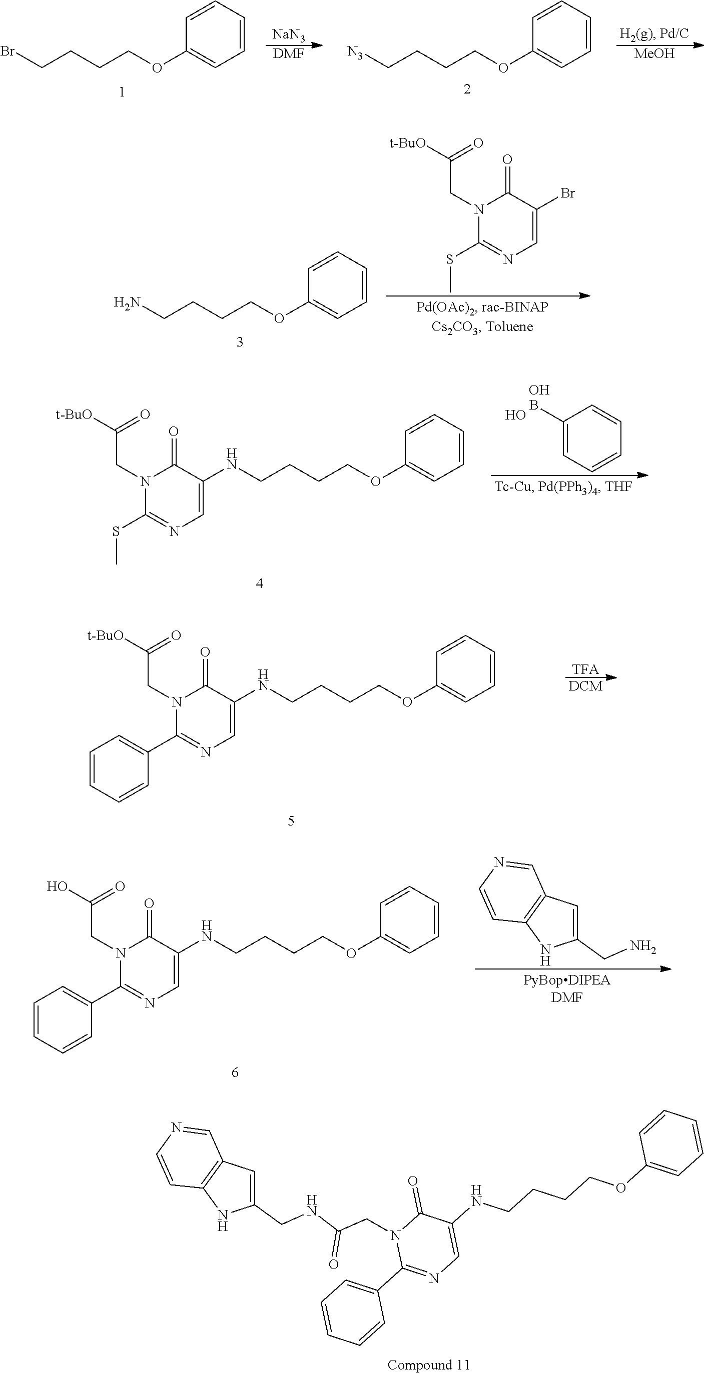

### Step 1: (4-azidobutoxy)benzene

To a solution of (4-bromobutoxy)benzene (200 mg, 0.88 mmol) in DMF (3 mL) was added NaN₃ (112 mg, 1.75 mmol) under N₂ atmosphere at 0° C. and the mixture was stirred at 50° C. for 16 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-8% EtOAc in PE) to give (4-azidobutoxy)benzene (150 mg, yield 93.0%) as a colorless oil. LC/MS (ESI) m/z: 192 (M+H)⁺.

### Step 2: 4-Phenoxybutan-1-amine

To a solution of (4-azidobutoxy)benzene (150 mg, 0.78 mmol) in MeOH (3 mL) was added Pd/C (20 mg, 10% wt.), the mixture was degassed under N₂ atmosphere for three times and stirred under a H₂ balloon at 25° C. overnight. The mixture was filtered, and the filtrate was concentrated to dryness to give 4-phenoxybutan-1-amine (120 mg, yield 99%) as a colorless oil, which was used directly in the next step without further purification. LC/MS (ESI) m/z: 166 (M+H)⁺.

### Step 3: Tert-butyl 2-(2-(methylthio)-6-oxo-5-((4-phenoxybutyl)amino)pyrimidin-1(6H)-yl)acetate

To a mixture of 4-phenoxybutan-1-amine (120 mg, 0.73 mmol) and tert-butyl 2-(5-bromo-2-(methylthio)-6-oxopyrimidin-1(6H)-yl)acetate (253 mg, 0.73 mmol) in toluene (3 mL) was added Cs₂CO₃ (494 mg, 1.46 mmol), rac-BINAP (94.3 mg, 0.15 mmol) and Pd(OAc)₂ (17 mg, 0.07 mmol) under N₂ atmosphere, the reaction mixture was degassed under N₂ atmosphere for three times and stirred at 120° C. overnight. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (PE:EtOAc=1:1) to give tert-butyl 2-(2-(methylthio)-6-oxo-5-((4-phenoxybutyl) amino)pyrimidin-1(6H)-yl)acetate (100 mg, yield 32.8%) as a yellow solid. LC/MS (ESI) m/z: 420 (M+H)⁺.

### Step 4: Tert-butyl 2-(6-oxo-5-((4-phenoxybutyl)amino)-2-phenylpyrimidin-1(6H)-yl)acetate

To a mixture of tert-butyl 2-(2-(methylthio)-6-oxo-5-((4-phenoxybutyl)amino) pyrimidin-1(6H)-yl)acetate (100 mg, 0.24 mmol) and phenylboronic acid (58 mg, 0.48 mmol) in THF (2 mL) was added ((thiophene-2-carbonyl)oxy)copper (100 mg, 0.53 mmol) and Pd(PPh₃)₄ (55 mg, 0.05 mmol) under N₂ atmosphere, the reaction mixture was degassed under N₂ atmosphere for three times and stirred at 55° C. overnight. The mixture was diluted with water and extracted with EtOAc twice. The combined organic layers were washed with saturated aq. NaHCO₃ and brine, dried over anhydrous Na₂SO₄, filtered, and concentrated under reduced pressure to dryness. The residue was purified by prep-TLC (PE:EtOAc=1:1) to give tert-butyl 2-(6-oxo-5-((4-phenoxybutyl)amino)-2-phenylpyrimidin-1(6H)-yl)acetate (30 mg, yield 28.0%) as a yellow solid. LC/MS (ESI) m/z: 450 (M+H)⁺.

### Step 5: 2-(6-oxo-5-((4-phenoxybutyl)amino)-2-phenylpyrimidin-1(6H)-yl)acetic acid

To a solution of tert-butyl 2-(6-oxo-5-((4-phenoxybutyl)amino)-2-phenylpyrimidin-1(6H)-yl)acetate (30 mg, 0.07 mmol) in DCM (2 mL) was added TFA (1 mL) and the mixture was stirred under N₂ atmosphere at room temperature overnight. The reaction mixture was concentrated under reduced pressure to dryness to give 2-(6-oxo-5-((4-phenoxybutyl)amino)-2-phenylpyrimidin-1(6H)-yl)acetic acid (25 mg, yield 95.2%) as a yellow solid, which was used directly in the next step without further purification. LC/MS (ESI) m/z: 394 (M+H)⁺.

### Step 6: N-((1H-pyrrolo[3,2-c]pyridin-2-yl)methyl)-2-(6-oxo-5-((4-phenoxybutyl) amino)-2-phenylpyrimidin-1(6H)-yl)acetamide (Compound 11)

To a mixture of 2-(6-oxo-5-((4-phenoxybutyl)amino)-2-phenylpyrimidin-1(6H)-yl)acetic acid (25 mg, 0.06 mmol) and (1H-pyrrolo[3,2-c]pyridin-2-yl)methanamine (19 mg, 0.12 mmol) in DMF (2 mL) was added DIPEA (49 mg, 0.36 mmol) and PyBop (33 mg, 0.06 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=12:1) and further purified by prep-HPLC to give Compound 11 (4.0 mg, yield 12.1%) as a white solid. ¹H NMR (400 MHz, CD₃OD) δ 8.73 (s, 1H), 8.12 (d, J=5.9 Hz, 1H), 7.49 (s, 1H), 7.47 (d, J=1.7 Hz, 2H), 7.45 (d, J=4.8 Hz, 1H), 7.42 (d, J=5.5 Hz, 1H), 7.40 (s, 1H), 7.27-7.23 (m, 2H), 7.18 (s, 1H), 6.93-6.89 (m, 3H), 6.52 (s, 1H), 4.62 (s, 2H), 4.55 (s, 2H), 4.05 (d, J=5.8 Hz, 2H), 3.27 (s, 2H), 1.90 (d, J=2.9 Hz, 4H). LC/MS (ESI) m/z: 523 (M+H)⁺. RT (Method A): 1.41 min.

The following compounds were prepared based on Steps 3-6 of Scheme 4:

| # | Reactant A | Reactant B | Reactant C | Reactant D | Characterization Data |
|---|---|---|---|---|---|
| 1 | 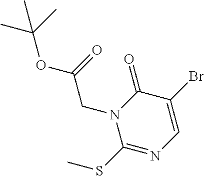 [SMILES: CSc1ncc(Br)c(=O)n1CC(=O)OC(C)(C)C] | 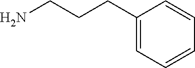 | 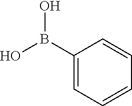 | 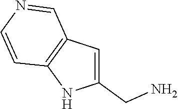 [SMILES: NCc1cc2cnccc2[nH]1] | ¹H NMR (400 MHz, CD₃OD) δ 9.05 (s, 1H), 8.31 (dd, J = 6.7, 0.8 Hz, 1H), 7.90 (d, J = 6.7 Hz, 1H), 7.52 (d, J = 7.4 Hz, 3H), 7.46 (d, J = 6.7 Hz, 2H), 7.27 (dd, J = 10.0, 4.6 Hz, 2H), 7.22 (d, J = 6.7 Hz, 2H), 7.17 (t, J = 7.1 Hz, 1H), 7.09 (s, 1H), 6.90 (s, 1H), 4.65 (d, J= 12.0 Hz, 4H), 3.19 (t, J = 7.0 Hz, 2H), 2.77-2.72 (m, 2H), 2.03- 1.96 (m, 2H). LC/MS (ESI) m/z: |
| | | | | | 493 (M + H)⁺. RT (Method A): 1.42 |
| | | | | | min. |
| 2 | 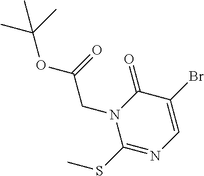 | 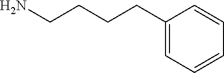 [SMILES: NCCCCc1ccccc1] | 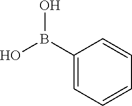 | 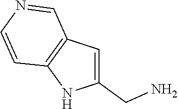 [SMILES: NCc1cc2cnccc2[nH]1] | ¹H NMR (400 MHz, CD₃OD) δ 9.03 (s, 1H), 8.28 (d, J = 6.6 Hz, 1H), 7.88 (d, J = 6.7 Hz, 1H), 7.52- 7.47 (m, 3H), 7.46 -7.40 (m, 2H), 7.23 (dt, J = 18.1, 7.1 Hz, 4H), 7.16-7.12 (m, 2H), 6.89 (d, J = 0.7 Hz, 1H), 4.64 (d, J = 6.6 Hz, 4H), 3.19 (t, J = 6.6 Hz, 2H), 2.68 (t, J = 7.2 Hz, 2H), 1.79- 1.68 (m, 4H). LC/MS (ESI) m/z: 507 (M + H)⁺. RT (Method A): 1.44 |
| | | | | | min. |
| 3 | 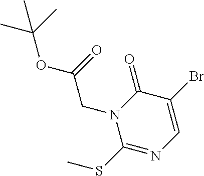 [SMILES: CSc1ncc(Br)c(=O)n1CC(=O)OC(C)(C)C] | 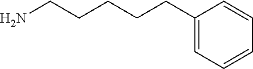 [SMILES: NCCCCCc1ccccc1] | 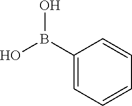 | 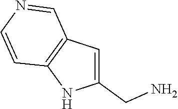 [SMILES: NCc1cc2cnccc2[nH]1] | ¹H NMR (400 MHz, CD₃OD) δ 9.04 (s, 1H), 8.30 (d, J = 6.7 Hz, 1H), 7.89 (d, J = 6.7 Hz, 1H), 7.50 (dd, J = 6.7, 4.2 Hz, 3H), 7.46- 7.41 (m, 2H), 7.24 (t, J = 7.4 Hz, 2H), 7.15 (dd, J = 13.7, 7.2 Hz, 4H), 6.89 (s, 1H), 4.64 (d, J = 7.5 Hz, 4H), 3.16 (t, J = 7.0 Hz, 2H), 2.63 (t, J = 7.6 Hz, 2H), 1.70 (dd, J = 15.6, 7.7 Hz, 4H), 1.47 (t, J = 7.8 Hz, 2H). LC/MS (ESI) m/z: |
| | | | | | 521 (M + H)⁺. RT (Method A): 1.57 |
| | | | | | min. |
| 6 | 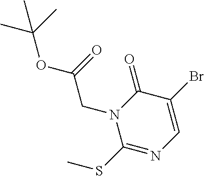 | 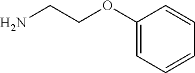 | 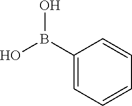 | 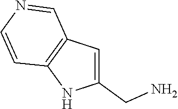 [SMILES: NCc1cc2cnccc2[nH]1] | ¹H NMR (400 MHz, CD₃OD) δ 8.89 (s, 1H), 8.46 (s, 1H), 8.19 (d, J = 6.3 Hz, 1H), 7.67 (d, J = 6.3 Hz, 1H), 7.49 (td, J = 6.7, 1.4 Hz, 3H), 7.45-7.40 (m, 2H), 7.35 (s, 1H), 7.29-7.25 (m, 2H), 6.97- 6.92 (m, 3H), 6.72 (s, 1H), 4.62 (d, J = 19.6 Hz, 4H), 4.22 (t, J = 5.2 Hz, 2H), 3.60 (t, J = 5.2 Hz, 2H). LC/MS (ESI) m/z: 495 (M + H)⁺. RT (Method A): 1.21 min. |
| 7 | 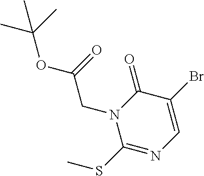 | 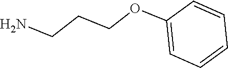 [SMILES: NCCCOc1ccccc1] | 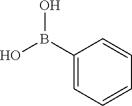 | 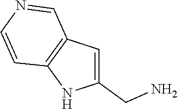 [SMILES: NCc1cc2cnccc2[nH]1] | ¹H NMR (400 MHz, CD₃OD) δ 8.87 (s, 1H), 8.48 (s, 1H), 8.20 (d, J = 6.2 Hz, 1H), 7.65 (d, J = 6.3 Hz, 1H), 7.50-7.46 (m, 3H), 7.43- 7.39 (m, 2H), 7.26-7.21 (m, 3H), 6.96-6.88 (m, 3H), 6.70 (s, 1H), 4.62 (d, J = 18.9 Hz, 4H), 4.12 (t, J = 5.8 Hz, 2H), 3.39 (t, J = 6.6 Hz, 2H), 2.15 (p, J = 6.3 Hz, 2H). LC/MS (ESI) m/z: 509 (M + H)⁺. RT (Method A): 1.31 min. |
| 37ª | 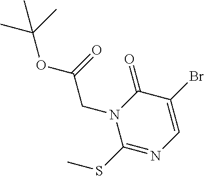 | 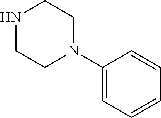 [SMILES: c1ccc(N2CCNCC2)cc1] | 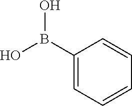 | 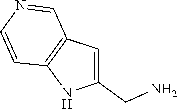 [SMILES: NCc1cc2cnccc2[nH]1] | ¹H NMR (400 MHz, CD₃OD) δ 8.86 (s, 1H), 8.20 (d, J = 6.3 Hz, 1H), 7.61 (d, J = 7.3 Hz, 2H), 7.53 (t, J = 8.3 Hz, 3H), 7.46 (t, J = 7.5 Hz, 2H), 7.27 (t, J = 8.0 Hz, 2H), 7.04 (d, J = 8.3 Hz, 2H), 6.88 (t, J = 7.1 Hz, 1H), 6.68 (s, 1H), 4.62 (d, J = 17.0 Hz, 4H), 3.34 (s, 8H). LC/MS (ESI) m/z: 520 (M + H)⁺. RT (Method A): 1.19 min. |
| 44^{b} | 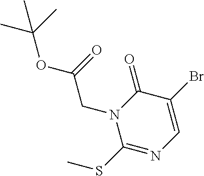 | 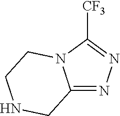 [SMILES: FC(F)(F)c1nnc2n1CCNC2] | 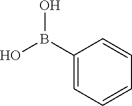 | 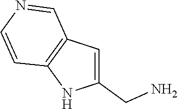 [SMILES: NCc1cc2cnccc2[nH]1] | ¹H NMR (400 MHz, CD₃OD) δ 8.73 (s, 1H), 8.12 (d, J = 5.9 Hz, 1H), 7.74 (s, 1H), 7.55-7.49 (m, 3H), 7.46-7.39 (m, 3H), 6.51 (s, 1H), 4.70 (s, 2H), 4.66 (s, 2H), 4.56 (s, 2H), 4.37 (t, J = 5.4 Hz, 2H), 3.82 (t, J = 5.4 Hz, 2H). LC/MS (ESI) m/z: 550 (M + H)⁺. RT (Method A): 0.65 min. |
| 61^{b} | 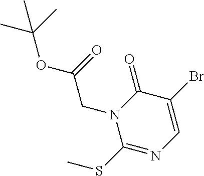 | 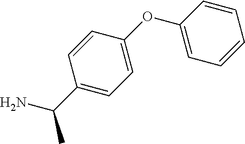 | 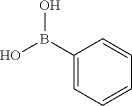 | 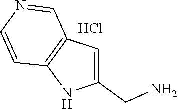 [SMILES: NCc1cc2cnccc2[nH]1 ; Cl] | ¹H NMR (400 MHz, CD₃OD) δ 8.75 (s, 1H), 8.13 (d, J = 5.3 Hz, 1H), 7.43 (d, J = 6.4 Hz, 1H), 7.37 (d, J = 7.0 Hz, 1H), 7.33 (s, 1H), 7.09 (s, 1H), 6.96 (s, 1H), 6.88 (s, 1H), 6.52 (s, 1H), 4.62 (s, 1H), 4.55 (s, 1H), 4.50 (d, J = 5.1 Hz, 1H), 1.59 (d, J = 5.9 Hz, 1H). LC/MS (ESI) m/z: 571 (M + H)⁺. RT (Method A): 1.76 min. |
| 62^{b,c} | 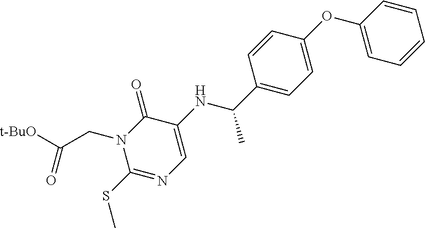 | | 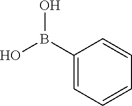 | 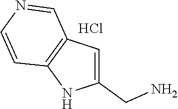 [SMILES: NCc1cc2cnccc2[nH]1 ; Cl] | ¹H NMR (400 MHz, CD₃OD) δ 8.73 (s, 1H), 8.12 (s, 1H), 7.45- 7.31 (m, 10H), 7.09 (t, J = 7.4 Hz, 1H), 6.96 (s, 4H), 6.88 (s, 1H), 6.50 (s, 1H), 4.62 (s, 2H), 4.54 (s, 2H), 4.50 (d, J = 5.9 Hz, 1H), 1.59 (d, J = 4.6 Hz, 3H). LC/MS (ESI) m/z: 571 (M + H)⁺. RT (Method A): 1.76 min. |
| 65^{b,d} | 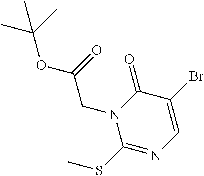 | 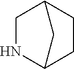 | 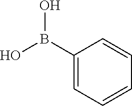 | 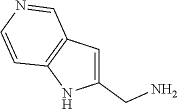 [SMILES: NCc1cc2cnccc2[nH]1] | ¹H NMR (400 MHz, CD₃OD) δ 8.72 (s, 1H), 8.12 (d, J = 4.0 Hz, 1H), 7.49-7.45 (m, 3H), 7.41 (d, J = 7.6 Hz, 3H), 7.09 (s, 1H), 6.52 (s, 1H), 4.68 (s, 1H), 4.56 (s, 4H), 3.66 (dd, J = 6.6, 2.8 Hz, 1H), 2.95 (d, J = 9.4 Hz, 1H), 2.58 (s, 1H), 1.82-1.75 (m, 2H), 1.71 (d, J = 16.1 Hz, 2H), 1.52 (d, J = 9.3 Hz, 1H), 1.37 (d, J = 5.4 Hz, 1H). LC/MS (ESI) m/z: 455 (M + H)⁺. |
| | | | | | RT (Method A): 1.03 min. |

| | | | | |
|---|---|---|---|---|
| 216^{b,c,d} | 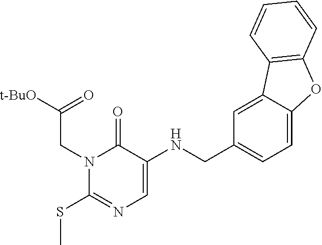 | 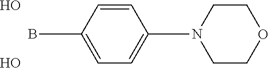 | 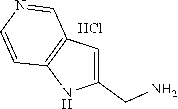 [SMILES: NCc1cc2cnccc2[nH]1 ; Cl] | ¹H NMR (400 MHz, CD₃OD) δ 8.99 (s, 1H), 8.27 (d, J = 6.3 Hz, 1H), 8.07 (s, 1H), 8.01-7.98 (m, 1H), 7.84 (d, J = 6.2 Hz, 1H), 7.55 (d, J = 4.0 Hz, 2H), 7.50-7.46 (m, 1H), 7.34 (d, J = 8.7 Hz, 3H), 7.12 (s, 1H), 6.92-6.87 (m, 4H), 4.70 (s, 2H), 4.65 (s, 2H), 4.59 (s, 2H), 3.82-3.79 (m, 4H), 3.15-3.12 (m, 4H). LC/MS (ESI) m/z: 640 M + H)⁺. RT (Method A): 1.63 min. |

^{a}HBTU was used in place of PyBOP in Step 6.
^{b}HATU was used in place of PyBOP in Step 6.
^{c}Steps 4-6 only.
^{d}The intermediate obtained from Step 4 was deprotected with NaOH.

### Scheme 5. Synthesis of N-(1-(2-(((1H-pyrrolo[3,2-c]pyridin-2-yl)methyl)amino)-2-oxoethyl)-6-oxo-2-phenyl-1,6-dihydropyrimidin-5-yl)-4-(4-phenoxyphenyl) butanamide (Compound 1.8)

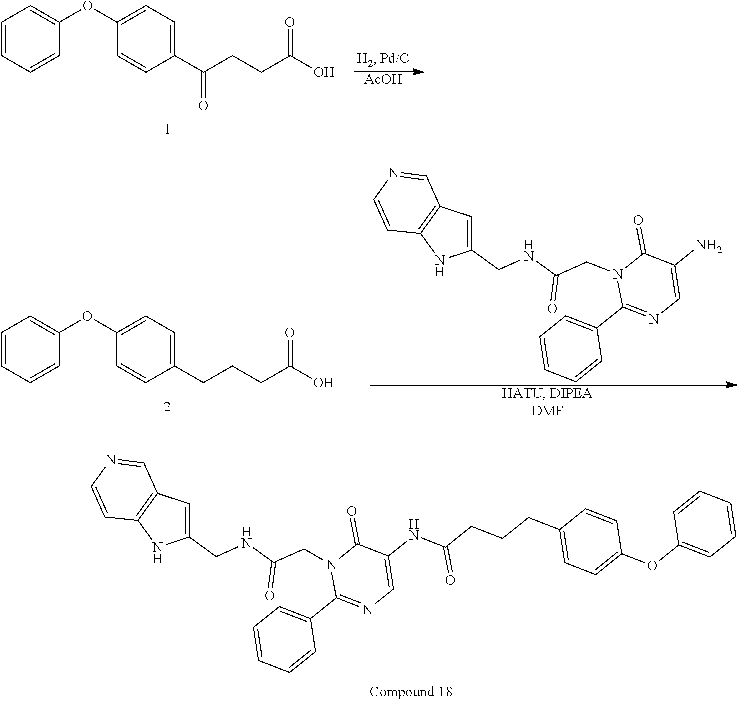

### Step 1: 4-(4-phenoxyphenyl)butanoic acid

To a solution of 4-oxo-4-(4-phenoxyphenyl)butanoic acid (1.0 g, 3.70 mmol) in AcOH (110 mL) was added Pd/C (80 mg, 10% wt.), the mixture was degassed under N₂ atmosphere for three times and the mixture was stirred under a H₂ balloon at 70° C. for 5 hours. The mixture was filtered, and filtrate was concentrated under reduced pressure to dryness to give 4-(4-phenoxyphenyl)butanoic acid (880 mg, yield 92.8%) as a yellow oil, which was used directly in the next step without further purification. LC/MS (ESI) (m/z): 257 (M+H)⁺.

### Step 2: N-(1-(2-(((1H-pyrrolo[3,2-c]pyridin-2-yl)methyl)amino)-2-oxoethyl)-6-oxo-2-phenyl-1,6-dihydropyrimidin-5-yl)-4-(4-phenoxyphenyl)butanamide (Compound 18)

To a mixture of 4-(4-phenoxyphenyl)butanoic acid (40 mg, 0.15 mmol) and N-((1H-pyrrolo[3,2-c]pyridin-2-yl)methyl)-2-(5-amino-6-oxo-2-phenylpyrimidin-1(6H)-yl) acetamide (58 mg, 0.15 mmol) in DMF (2 mL) was added DIPEA (120 mg, 0.90 mmol) and HATU (71 mg, 0.18 mmol) under N₂ atmosphere and the reaction mixture was stirred at room temperature for 2 hours. The mixture was diluted with EtOAc, washed with saturated aq. NaHCO₃ solution and brine, dried over anhydrous Na SO₄, filtered, and concentrated under reduced pressure to dryness. The residue was purified by prep-TLC (DCM:MeOH=10:1) and further purified by prep-HPLC to give Compound 18 (10 mg, yield 10.5%) as a white solid. ¹H NMR (400 MHz, CD₃OD) δ 8.92 (s, 1H), 8.73 (s, 1H), 8.12 (d, J=5.9 Hz, 1H), 7.52 (d, J=8.1 Hz, 3H), 7.43 (t, J=5.5 Hz, 3H), 7.34-7.30 (m, 2H), 7.22 (d, J=8.5 Hz, 2H), 7.07 (t, J=7.4 Hz, 1H), 6.93 (dd, J=14.5, 8.1 Hz, 4H), 6.52 (s, 1H), 4.67 (s, 2H), 4.55 (s, 2H), 2.70 (t, J=7.6 Hz, 2H), 2.53 (t, J=7.3 Hz, 2H), 2.04-2.00 (m, 2H). LC/MS (ESI) m/z: 613 (M+H)⁺. RT (Method A): 1.68 min.

### Scheme 6. Synthesis of N-((1H-pyrrolo[3,2-c]pyridin-2-yl)methyl)-2-(6-oxo-5-((4-(4-phenoxyphenyl)butyl)amino)-2-phenylpyrimidin-1(6H)-yl)acetamide (Compound 21)

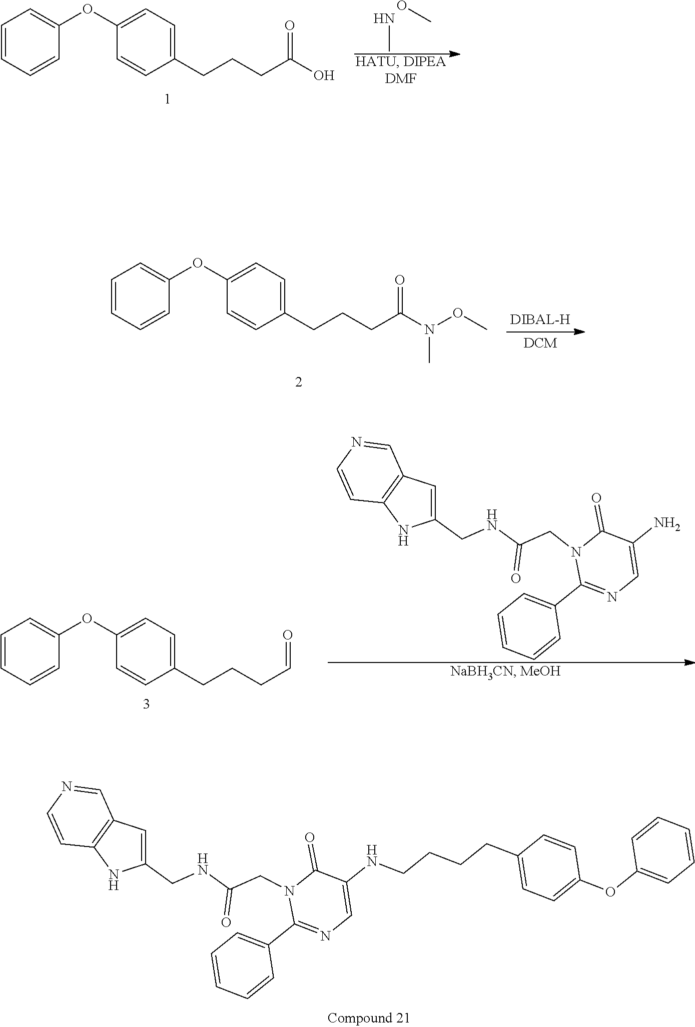

### Step 1: N-methoxy-N-methyl-4-(4-phenoxyphenyl)butanamide

To a mixture of 4-(4-phenoxyphenyl)butanoic acid (300 mg, 1.17 mmol) and N,O-dimethylhydroxylamine (229 mg, 2.34 mmol) in DMF (3 mL) was added DIPEA (907 mg, 7.02 mmol) and HATU (534 mg, 1.40 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-35% EtOAc in PE) to give N-methoxy-N-methyl-4-(4-phenoxyphenyl)butanamide (242 mg, yield 69.1%) as a brown oil. LC/MS (ESI) m/z: 300 (M+H)⁺.

### Step 2: 4-(4-Phenoxyphenyl)butanal

To a solution of N-methoxy-N-methyl-4-(4-phenoxyphenyl)butanamide (242 mg, 0.81 mmol) in DCM (3 mL) was added DIBAL-H (1.20 mL, 1.22 mmol, 1 N in THF) drop-wisely under N₂ atmosphere at −78° C. and the mixture was stirred at −78° C. for 1 hour. The mixture was quenched with saturated aq. Potassium sodium tartrate solution and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give 4-(4-phenoxyphenyl)butanal (80 mg, yield 41.2%) as a colorless oil. LC/MS (ESI) m/z: 241 (M+H)⁺.

### Step 3: N-((1H-pyrrolo[3,2-c]pyridin-2-yl)methyl)-2-(6-oxo-5-((4-(4-phenoxyphenyl) butyl)amino)-2-phenylpyrimidin-1(6H)-yl)acetamide (Compound 21)

To a mixture of N-((1H-pyrrolo[3,2-c]pyridin-2-yl)methyl)-2-(5-amino-6-oxo-2-phenylpyrimidin-1(6H)-yl)acetamide (30 mg, 0.08 mmol) and 4-(4-phenoxyphenyl)butanal (38 mg, 0.16 mmol) in MeOH (3 mL) was added NaBH₃CN (20 mg, 0.32 mmol) under N₂ atmosphere and the reaction mixture was stirred at 50° C. for 1 hour. The mixture was diluted with EtOAc, washed with brine, dried over anhydrous Na₂SO₄, filtered, and concentrated under reduced pressure to dryness. The residue was purified by prep-TLC (DCM:MeOH=15:1) and further purified by prep-HPLC to give Compound 21 (12 mg, yield 25.0%) as a white solid. ¹H NMR (400 MHz, CD₃OD) δ 8.97 (s, 1H), 8.26 (d, J=6.5 Hz, 1H), 7.79 (d, J=6.5 Hz, 1H), 7.49 (t, J=5.9 Hz, 3H), 7.45-7.40 (m, 2H), 7.30 (t, J=7.9 Hz, 2H), 7.19 (d, J=8.4 Hz, 2H), 7.14 (s, 1H), 7.06 (t, J=7.4 Hz, 1H), 6.91 (dd, J=16.2, 8.2 Hz, 4H), 6.81 (s, 1H), 4.63 (d, J=13.1 Hz, 4H), 3.20 (t, J=6.4 Hz, 2H), 2.67 (t, J=7.0 Hz, 2H), 1.74 (dt, J=8.4, 4.9 Hz, 4H). LC/MS (ESI) m/z: 599 (M+H)⁺. RT (Method A): 1.94 min.

### Scheme 7. Synthesis of N-(1-(2-(((1H-pyrrolo[3,2-c]pyridin-2-yl)methyl)amino)-2-oxoethyl)-6-oxo-2-phenyl-1,6-dihydropyrimidin-5-yl)-4-oxo-4-(4-phenoxyphenyl butanamide (Compound 23)

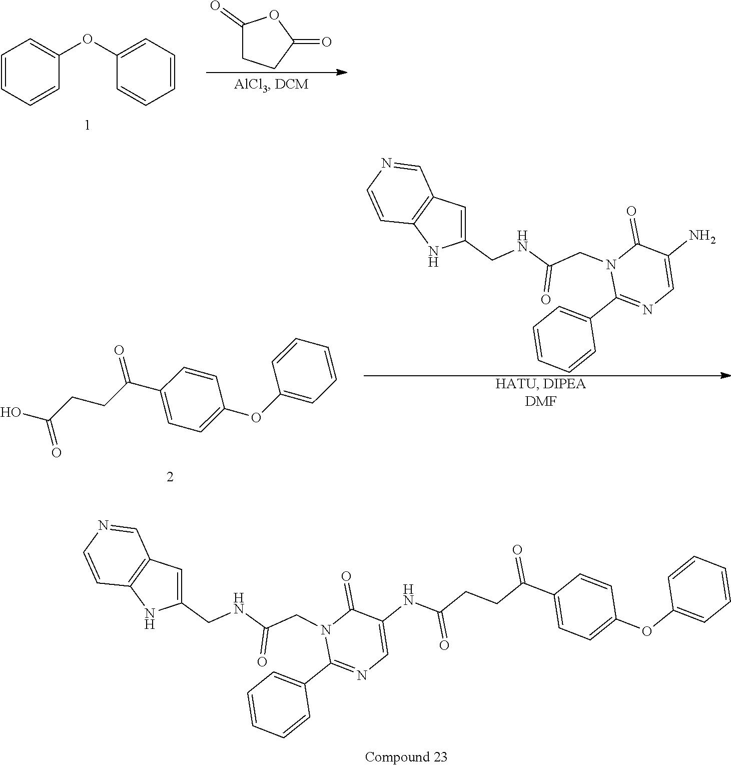

### Step 1: 4-oxo-4-(4-phenoxyphenyl)butanoic acid

To a mixture of oxydibenzene (5.0 g, 29.4 mmol) and dihydrofuran-2,5-dione (2.9 g, 29.4 mmol) in DCM (50 mL) was added AlCl₃ (5.9 g, 44.1 mmol) under N₂ atmosphere at 0° C. and the mixture was stirred at room temperature overnight. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give 4-oxo-4-(4-phenoxyphenyl)butanoic acid (7.8 g, yield 98.2%) as a white solid. LC/MS (ESI) (m/z): 271 (M+H)⁺.

### Step 2: N-(1-(2-(((1H-pyrrolo[3,2-c]pyridin-2-yl)methyl)amino)-2-oxoethyl)-6-oxo-2-phenyl-1,6-dihydropyrimidin-5-yl)-4-oxo-4-(4-phenoxyphenyl)butanamide (Compound 23)

To a mixture of 4-oxo-4-(4-phenoxyphenyl)butanoic acid (40 mg, 0.15 mmol) and N-((1H-pyrrolo[3,2-c]pyridin-2-yl)methyl)-2-(5-amino-6-oxo-2-phenylpyrimidin-1(6H)-yl)acetamide (56 mg, 0.15 mmol) in DMF (2 mL) was added DIPEA (120 mg, 0.90 mmol) and HATU (72 mg, 0.19 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=10:1) and further purified by prep-HPLC to give Compound 23 (4.3 mg, yield 4.63%) as a white solid. ¹H NMR (400 MHz, CD₃OD) δ 8.91 (s, 1H), 8.75 (s, 1H), 8.14 (d, J=5.9 Hz, 1H), 8.04 (d, J=8.8 Hz, 2H), 7.53 (d, J=8.3 Hz, 3H), 7.46-7.41 (m, 5H), 7.24 (d, J=7.4 Hz, 1H), 7.09 (d, J=7.8 Hz, 2H), 7.02 (d, J=8.8 Hz, 2H), 6.54 (s, 1H), 4.68 (s, 2H), 4.56 (s, 2H), 3.41 (t, J=6.4 Hz, 2H), 2.92 (t, J=6.4 Hz, 2H). LC/MS (ESI) m/z: 627 (M+H)⁺. RT (Method A): 1.59 min.

### Scheme 7. Synthesis of N-((1H-pyrrolo[3,2-c]pyridin-2-yl)methyl)-2-(6-oxo-2,5-diphenylpyrimidin-1(6H)-yl)acetamide (Compound 25)

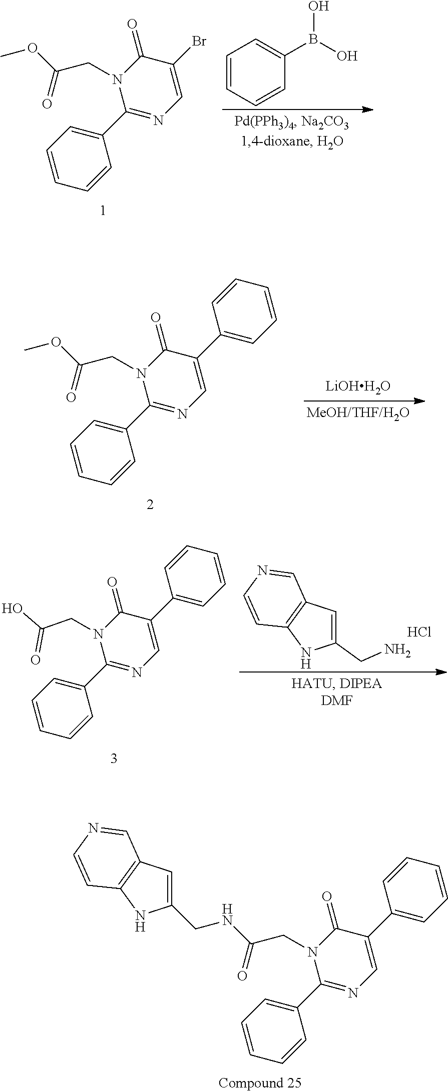

### Step 1: Methyl 2-(6-oxo-2,5-diphenylpyrimidin-1(6H)-yl)acetate

To a mixture of methyl 2-(5-bromo-6-oxo-2-phenylpyrimidin-1(6H)-yl)acetate (40 mg, 0.12 mmol) and phenylboronic acid (23 mg, 0.18 mmol) in 1,4-dioxane (1.5 mL) and water (1.5 mL) was added Na₂CO₃ (32 mg, 0.30 mmol) and Pd(PPh₃)₄ (15 mg, 0.01 mmol) under N₂ atmosphere, the reaction mixture was degassed under N₂ atmosphere for three times and stirred at 80° C. for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-70% EtOAc in PE) to give methyl 2-(6-oxo-2,5-diphenylpyrimidin-1(6H)-yl)acetate (39 mg, yield 98.1%) as a white solid. LC/MS (ESI) m/z: 321 (M+H)⁺.

### Step 2: 2-(6-Oxo-2,5-diphenylpyrimidin-1(6H)-yl)acetic acid

To a solution of methyl 2-(6-oxo-2,5-diphenylpyrimidin-1(6H)-yl)acetate (39 mg, 0.15 mmol) in MeOH/THF/H₂O (4 mL, 2/1/1) was added LiOH·H₂O (15 mg, 0.30 mmol) under N₂ atmosphere and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to give 2-(6-oxo-2,5-diphenylpyrimidin-1(6H)-yl)acetic acid (37 mg, yield 99.2%) as a white solid, which was used directly in the next step without further purification. LC/MS (ESI) m/z: 307 (M+H)⁺.

### Step 3: N-((1H-pyrrolo[3,2-c]pyridin-2-yl)methyl)-2-(6-oxo-2,5-diphenylpyrimidin-1(6H)-yl)acetamide (Compound 25)

To a mixture of 2-(6-oxo-2,5-diphenylpyrimidin-1(6H)-yl)acetic acid (35 mg, 0.11 mmol) and (1H-pyrrolo[3,2-c]pyridin-2-yl)methanamine hydrochloride (20 mg, 0.11 mmol) in DMF (2 mL) was added DIPEA (78 mg, 0.55 mmol) and HATU (54 mg, 0.14 mmol) under N₂ atmosphere and the reaction mixture was stirred at room temperature for 1 hour. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=10:1) and further purified by prep-HPLC to give Compound 25 (1.1 mg, yield 2.21%) as a white solid. ¹H NMR (400 MHz, CD₃OD) δ 8.68 (s, 1H), 8.52 (s, 1H), 8.45-8.41 (m, 1H), 8.23 (d, J=7.2 Hz, 2H), 8.10 (d, J=6.5 Hz, 1H), 7.76 (d, J=7.1 Hz, 2H), 7.48 (dd, J=9.5, 5.3 Hz, 3H), 7.43 (d, J=7.3 Hz, 1H), 7.33 (t, J=7.3 Hz, 1H), 7.27 (d, J=7.8 Hz, 2H), 6.64 (s, 1H), 5.08 (s, 2H), 4.64 (s, 2H). LC/MS (ESI) m/z: 436 (M+H)⁺. RT (Method A): 1.31 min.

### Scheme 8. Synthesis of N-((1H-pyrrolo[3,2-c]pyridin-2-yl)methyl)-2-(5-((dibenzo[b,d]furan-3-ylmethyl)amino)-6-oxo-2-phenylpyrimidin-1(6H)-yl)acetamide (Compound 26)

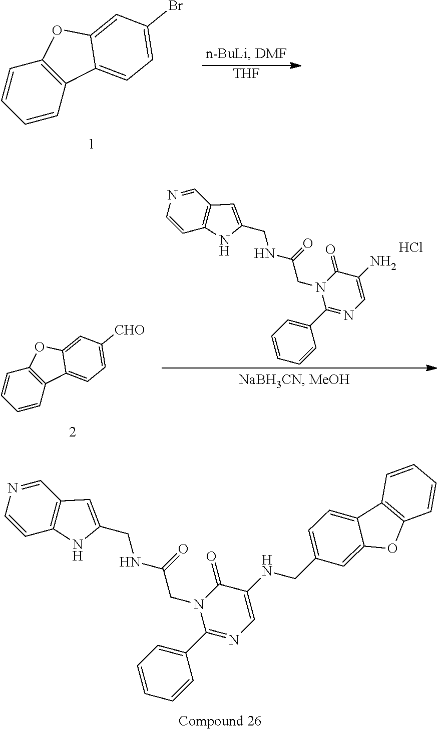 [SMILES: Brc1ccc2c(c1)oc1ccccc12 ; O=Cc1ccc2c(c1)oc1ccccc12 ; Nc1cnc(-c2ccccc2)n(CC(=O)NCc2cc3cnccc3[nH]2)c1=O ; Cl ; O=C(Cn1c(-c2ccccc2)ncc(NCc2ccc3c(c2)oc2ccccc23)c1=O)NCc1cc2cnccc2[nH]1 ; "THF"]

### Step 1: Dibenzo[b,d]furan-3-carbaldehyde

To a solution of 3-bromodibenzo[b,d]furan (300 mg, 1.21 mmol) in THF (10 mL) was added n-BuLi (0.8 mL, 2.02 mmol, 2.5 M in hexane) drop-wisely under N₂ atmosphere at −78° C., the reaction mixture was slowly warmed to 0° C. and stirred for 10 minutes. Then the reaction mixture was cooled down to −78° C., DMF (266 mg, 3.64 mmol) was added into the above mixture. The resulting mixture was stirred at −78° C. for 1 hour. The reaction mixture was quenched with saturated aq. NH₄Cl solution at 0° C. and extracted with EtOAc twice. The combined organic layers were washed with water and brine, dried over anhydrous Na₂SO₄, filtered, and concentrated under reduced pressure to dryness. The residue was purified by flash chromatography (silica gel, 0-20% EtOAc in PE) to give dibenzo[b,d]furan-3-carbaldehyde (30 mg, yield 12.6%) as a white solid. ¹H NMR (400 MHz, CDCl₃) δ 10.14 (s, 1H), 8.11-8.02 (m, 3H), 7.91 (dd, J=7.9, 1.3 Hz, 1H), 7.65-7.55 (m, 2H), 7.42 (dd, J=11.6, 4.3 Hz, 1H).

### Step 2: N-((1H-pyrrolo[3,2-c]pyridin-2-yl)methyl)-2-(5-((dibenzo[b,d]furan-3-ylmethyl)amino)-6-oxo-2-phenylpyrimidin-1(6H)-yl)acetamide (Compound 26)

To a mixture of dibenzo[b,d]furan-3-carbaldehyde (30 mg, 0.15 mmol) and N-((1H-pyrrolo[3,2-c]pyridin-2-yl)methyl)-2-(5-amino-6-oxo-2-phenylpyrimidin-1(6H)-yl) acetamide hydrochloride (21 mg, 0.05 mmol) in MeOH (5 mL) was added NaBH₃CN (12 mg, 0.19 mmol) under N₂ atmosphere and the reaction mixture was stirred at 50° C. for 2 hours. The mixture was diluted with EtOAc, washed with brine, dried over anhydrous Na₂SO₄, filtered, and concentrated under reduced pressure to dryness. The residue was purified by prep-TLC (DCM:MeOH=15:1) and further purified by prep-HPLC to give Compound 26 (5 mg, yield 17.6%) as a white solid. ¹H NMR (400 MHz, CD₃OD) δ 8.82 (d, J=5.7 Hz, 1H), 8.51 (s, 1H), 8.17 (t, J=6.8 Hz, 1H), 8.03-7.96 (m, 2H), 7.64 (d, J=5.8 Hz, 1H), 7.60-7.54 (m, 2H), 7.48-7.41 (m, 5H), 7.39-7.33 (m, 3H), 7.08 (s, 1H), 6.63 (d, J=6.2 Hz, 1H), 4.66-4.57 (m, 6H). LC/MS (ESI) m/z: 555 (M+H)⁺. RT (Method A): 1.61 min.

The following compounds were prepared based on Scheme 8:

| # | Reactant A | Reactant B | Characterization Data |
|---|---|---|---|
| 19^{a} | 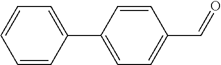 [SMILES: O=Cc1ccc(-c2ccccc2)cc1] | 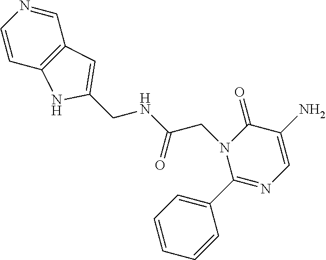 [SMILES: Nc1cnc(-c2ccccc2)n(CC(=O)NCc2cc3cnccc3[nH]2)c1=O] | ¹H NMR (400 MHz, CD₃OD) δ 8.64 (d, J = 70.2 Hz, 1H), 8.12 (d, J = 5.9 Hz, 1H), 7.60 (d, J = 7.8 Hz, 4H), 7.49-7.44 (m, 5H), 7.43 (d, J = 1.2 Hz, 1H), 7.36 (ddd, J = 20.6, 12.0, 5.8 Hz, 5H), 7.06 (s, 1H), 6.51 (s, 1H), 4.64 (s, 2H), 4.56 (s, 2H), 4.48 (s, 2H). LC/MS (ESI) m/z: 541 (M + H)⁺. RT (Method A): 1.57 min. |
| 20^{a} | 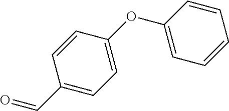 [SMILES: O=Cc1ccc(Oc2ccccc2)cc1] | 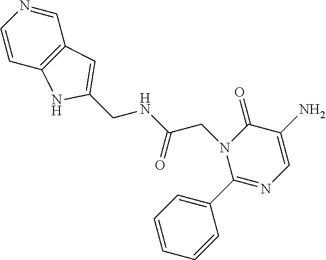 [SMILES: Nc1cnc(-c2ccccc2)n(CC(=O)NCc2cc3cnccc3[nH]2)c1=O] | ¹H NMR (400 MHz, CD₃OD) δ 8.79 (s, 1H), 8.52 (s, 1H), 8.16 (d, J = 6.1 Hz, 1H), 7.53 (d, J = 6.1 Hz, 1H), 7.46 (d, J = 7.8 Hz, 3H), 7.39 (dd, J = 7.4, 4.8 Hz, 4H), 7.33 (d, J = 7.7 Hz, 2H), 7.12-7.06 (m, 2H), 6.97 (dd, J = 8.6, 2.8 Hz, 4H), 6.59 (s, 1H), 4.64 (s, 2H), 4.57 (s, 2H), 4.41 (s, 2H). LC/MS (ESI) m/z: 557 (M + H)⁺. RT (Method A): 1.62 min. |
| 22 | 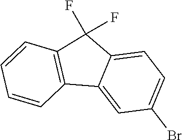 [SMILES: FC1(F)c2ccccc2-c2cc(Br)ccc21] | 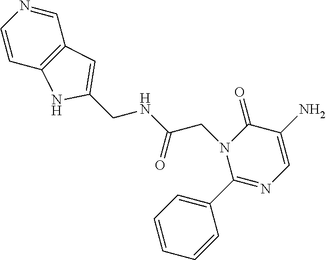 [SMILES: Nc1cnc(-c2ccccc2)n(CC(=O)NCc2cc3cnccc3[nH]2)c1=O] | ¹H NMR (400 MHz, CD₃OD) δ 8.78 (s, 1H), 8.53 (s, 1H), 8.15 (d, J = 6.0 Hz, 1H), 7.76 (s, 1H), 7.69 (d, J = 7.6 Hz, 1H), 7.58 (d, J = 6.6 Hz, 2H), 7.53- 7.49 (m, 2H), 7.44 (t, J = 8.3 Hz, 4H), 7.40-7.36 (m, 3H), 7.06 (s, 1H), 6.58 (s, 1H), 4.65 (s, 2H), 4.56 (d, J = 17.3 Hz, 4H). LC/MS (ESI) m/z: 589 (M + H)⁺. RT (Method A): 1.66 min. |
| 32ª | 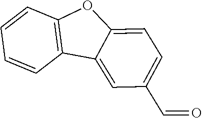 [SMILES: O=Cc1ccc2oc3ccccc3c2c1] | 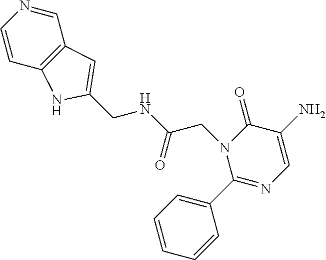 [SMILES: Nc1cnc(-c2ccccc2)n(CC(=O)NCc2cc3cnccc3[nH]2)c1=O] | ¹H NMR (400 MHz, CD₃OD) δ 8.77 (s, 1H), 8.14 (d, J = 6.0 Hz, 1H), 8.08 (s, 1H), 8.00 (d, J = 7.3 Hz, 1H), 7.56 (t, J = 5.8 Hz, 3H), 7.51-7.43 (m, 5H), 7.36 (dd, J = 15.2, 7.5 Hz, 3H), 7.12 (s, 1H), 6.58 (s, 1H), 4.64 (s, 2H), 4.58 (d, J = 8.0 Hz, 4H). LC/MS (ESI) m/z: 555 (M + H)⁺. RT (Method A): 1.61 min. |
| 43 | 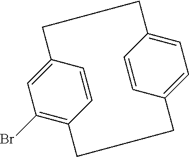 [SMILES: Brc1cc2ccc1CCc1ccc(cc1)CC2] | 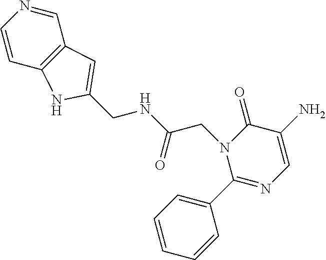 [SMILES: Nc1cnc(-c2ccccc2)n(CC(=O)NCc2cc3cnccc3[nH]2)c1=O] | ¹H NMR (400 MHz, CD₃OD) δ 8.74 (s, 1H), 8.13 (d, J = 5.9 Hz, 1H), 7.88 (d, J = 8.3 Hz, 2H), 7.46 (ddd, J = 20.6, 15.3, 7.7 Hz, 8H), 7.11 (s, 1H), 6.52 (s, 1H), 4.62 (s, 2H), 4.55 (s, 2H), 3.23 (t, J = 6.9 Hz, 2H), 3.10 (s, 3H), 2.92-2.85 (m, 2H), 2.09-2.00 (m, 2H). LC/MS (ESI) m/z: 595 (M + H)⁺. RT (Method A): 1.77 min. |
| 112 | 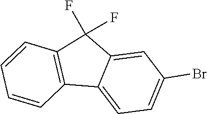 [SMILES: FC1(F)c2ccccc2-c2ccc(Br)cc21] | 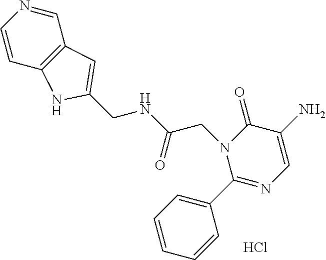 [SMILES: Nc1cnc(-c2ccccc2)n(CC(=O)NCc2cc3cnccc3[nH]2)c1=O ; Cl] | ¹H NMR (400 MHz, CD₃OD) δ 8.71 (s, 1H), 8.11 (d, J = 6.0 Hz, 1H), 7.69 (d, J = 9.5 Hz, 3H), 7.58 (t, J = 7.6 Hz, 2H), 7.52 (t, J = 7.6 Hz, 1H), 7.46 (d, J = 8.2 Hz, 3H), 7.41-7.35 (m, 4H), 7.07 (s, 1H), 6.49 (s, 1H), 4.64 (s, 2H), 4.55 (s, 2H), 4.51 (s, 2H). LC/MS (ESI) m/z: 589 (M + H)⁺. RT (Method A): 1.75 min. |
| 176ª | 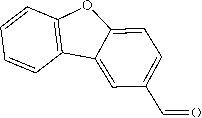 [SMILES: O=Cc1ccc2oc3ccccc3c2c1] | 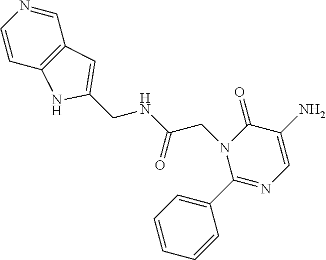 [SMILES: Nc1cnc(-c2ccccc2)n(CC(=O)NCc2cc3cnccc3[nH]2)c1=O] | ¹H-NMR (400 MHz, CD₃OD) δ 8.77 (s, 1H), 8.13 (d, J = 6.0 Hz, 1H), 8.06- 7.98 (m, 2H), 7.52 (dt, J = 16.9, 8.1 Hz, 5H), 7.40-7.32 (m, 6H), 6.76 (s, 1H), 6.56 (s, 1H), 4.80 (s, 2H), 4.55 (s, 4H). |
| 233^{a,b} | 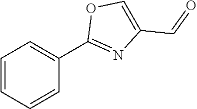 [SMILES: O=Cc1coc(-c2ccccc2)n1] | 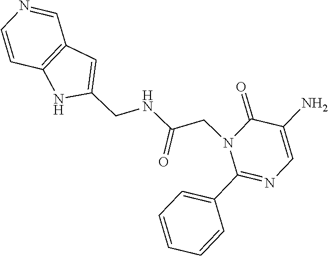 [SMILES: Nc1cnc(-c2ccccc2)n(CC(=O)NCc2cc3cnccc3[nH]2)c1=O] | ¹H NMR (400 MHz, CD₃OD) δ 8.70 (s, 1H), 8.10 (d, J = 5.9 Hz, 1H), 8.05- 8.00 (m, 2H), 7.93 (s, 1H), 7.52-7.45 (m, 6H), 7.41-7.36 (m, 3H), 7.30 (s, 1H), 6.47 (s, 1H), 4.63 (s, 2H), 4.54 (s, 2H), 4.41 (s, 2H). LC/MS (ESI) m/z: 532 (M + H)⁺. RT (Method A): 1.25 min. |

^{a}Step 2 only.
^{b}Step 2 was performed with NaBH₃CN in MeOH in the presence of MgSO₄.

Compound 178 can be prepared based on Step 2 in Scheme 8:

| # | Reactant A | Reactant B |
|---|---|---|
| 178 | 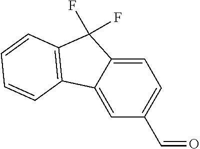 [SMILES: O=Cc1ccc2c(c1)-c1ccccc1C2(F)F] | 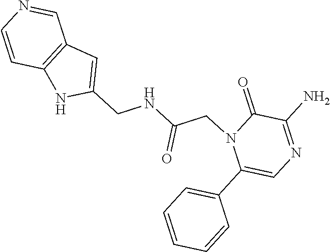 [SMILES: Nc1ncc(-c2ccccc2)n(CC(=O)NCc2cc3cnccc3[nH]2)c1=O] |

### Scheme 9. Synthesis of N-(1-(2-(((1H-pyrrolo[3,2-c]pyridin-2-yl)methyl)amino)-2-oxoethyl)-6-oxo-2-phenyl-1,6-dihydropyrimidin-5-yl)-[1,1′-biphenyl]-4-carboxamide_(Compound 29)

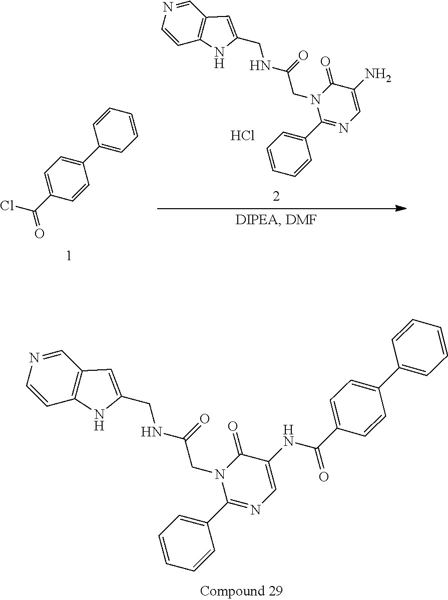 [SMILES: O=C(Cl)c1ccc(-c2ccccc2)cc1 ; Nc1cnc(-c2ccccc2)n(CC(=O)NCc2cc3cnccc3[nH]2)c1=O ; Cl ; O=C(Cn1c(-c2ccccc2)ncc(NC(=O)c2ccc(-c3ccccc3)cc2)c1=O)NCc1cc2cnccc2[nH]1]

To a mixture of [1,1′-biphenyl]-4-carbonyl chloride (22 mg, 0.1 mmol) and N-((1H-pyrrolo[3,2-c]pyridin-2-yl)methyl)-2-(5-amino-6-oxo-2-phenylpyrimidin-1(6H)-yl) acetamide hydrochloride (41 mg, 0.1 mmol) in DMF (0.5 mL) was added DIPEA (65 mg, 0.5 mmol) under N₂ atmosphere and the reaction mixture was stirred at room temperature for 2 hours. The mixture was purified by prep-HPLC to give Compound 29 (4.8 mg, yield 8.7%) as a white solid. ¹H NMR (400 MHz, CD₃OD) δ 9.08 (s, 1H), 8.77 (s, 1H), 8.14 (d, J=6.0 Hz, 1H), 8.07 (d, J=8.5 Hz, 2H), 7.83 (d, J=8.5 Hz, 2H), 7.75-7.70 (m, 2H), 7.60-7.56 (m, 2H), 7.55-7.52 (m, 1H), 7.50 (s, 1H), 7.49-7.40 (m, 5H), 6.56 (s, 1H), 4.74 (s, 2H), 4.58 (s, 2H). LC/MS (ESI) m/z: 555 (M+H)⁺. RT (Method A): 1.61 min.

Compound 31 was prepared based on Scheme 9:

| # | Reactant A | Reactant B | Characterization Data |
|---|---|---|---|
| 31 | 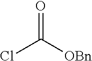 [SMILES: O=C(Cl)OCc1ccccc1] | 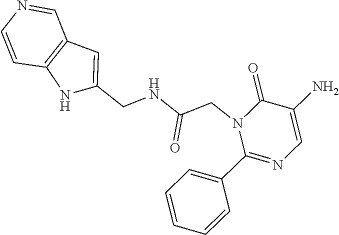 [SMILES: Nc1cnc(-c2ccccc2)n(CC(=O)NCc2cc3cnccc3[nH]2)c1=O] | ¹H NMR (400 MHz, CD₃OD) δ 8.79 (s, 1H), 8.33 (d, J = 6.0 Hz, 1H), 8.04 (d, J = 5.9 Hz, 1H), 7.58-7.54 (m, 2H), 7.46 (dd, J = 11.0, 5.0 Hz, 4H), 7.43-7.36 (m, 5H), 6.75 (s, 1H), 5.54 (s, 2H), 4.73 (s, 2H), 4.65 (s, 2H). LC/MS (ESI) m/z: 509 (M + H)⁺. RT (Method A): 1.15 min. |

### Scheme 10. Synthesis of N-((1H-pyrrolo[3,2-c]pyridin-2-yl)methyl)-2-(6-oxo-2-phenyl-5-(4,5,6,7-tetrahydrobenzo[d]thiazol-2-yl)pyrimidin-1(6H)-yl)acetamide (Compound 30)

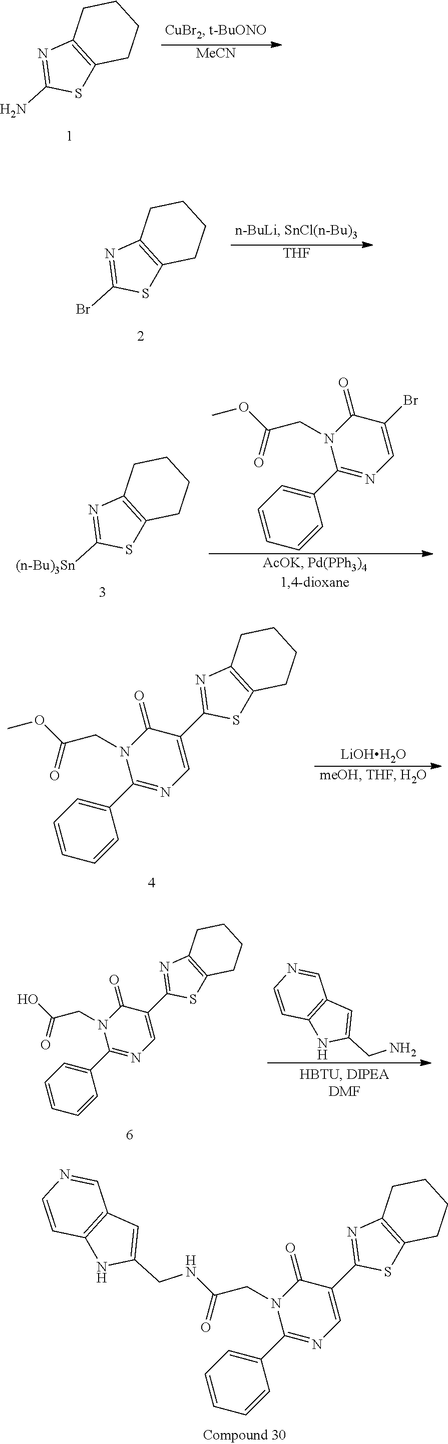

### Step 1: 2-Bromo-4,5,6,7-tetrahydrobenzo[d]thiazole (2)

To a solution of CuBr₂ (200 mg, 1.30 mmol) in MeCN (5 mL) was added tert-butyl nitrite (174 mg, 1.69 mmol) drop-wisely at 40° C. and the reaction mixture was stirred under N₂ atmosphere at 40° C. for 10 minutes. A solution of 4,5,6,7-tetrahydrobenzo[d]thiazol-2-amine (200 mg, 1.30 mmol) in MeCN (5 mL) was added to the mixture and the resulting mixture was stirred at 40° C. for 2 hours. The mixture was quenched with 0.5 N aq. HCl and extracted with EtOAc twice. The combined organic layers were washed with saturated aq. NaHCO₃ solution, brine, dried over anhydrous Na₂SO₄, filtered, and concentrated under reduced pressure to dryness. The residue was purified by flash chromatography (silica gel, 0-15% EtOAc in PE) to give 2-bromo-4,5,6,7-tetrahydrobenzo[d]thiazole (174 mg, yield 61.5%) as a light-yellow oil. LC/MS (ESI) m/z: 218 (M+H)⁺.

### Step 2: 2-(Tributylstannyl)-4,5,6,7-tetrahydrobenzo[d]thiazole (3)

At −78° C., to a solution of 2-bromo-4,5,6,7-tetrahydrobenzo[d]thiazole (77 mg, 0.35 mmol) in THF (3 mL) was added n-BuLi (0.6 mL, 0.40 mmol, 2.5 M) drop-wisely under N₂ atmosphere and the reaction mixture was stirred at −78° C. for 30 minutes. Tributylchlorostannane (104 mg, 0.32 mmol) was added to the mixture and the resulting mixture was stirred at −78° C. to room temperature for 1.5 hours. The mixture was concentrated under reduced pressure to dryness (15° C.). The residue was dissolved in hexane and the reaction mixture was stirred at room temperature for 5 minutes. The mixture was filtered through diatomite and the filtrate was concentrated under reduced pressure to dryness (15° C.) to give 2-(tributylstannyl)-4,5,6,7-tetrahydrobenzo[d]thiazole (150 mg, yield 99.2%) as a light-yellow oil, which was used directly in the next reaction without further purification.

### Step 3: Methyl 2-(6-oxo-2-phenyl-5-(4,5,6,7-tetrahydrobenzo[d]thiazol-2-yl) pyrimidin-1(6H)-yl)acetate (4)

To a mixture of 2-(tributylstannyl)-4,5,6,7-tetrahydrobenzo[d]thiazole (150 mg, 0.35 mmol) and methyl 2-(5-bromo-6-oxo-2-phenylpyrimidin-1(6H)-yl)acetate (77 mg, 0.24 mmol) in 1,4-dioxane (3 mL) was added AcOK (47 mg, 0.48 mmol) and Pd(PPh₃)₄ (28 mg, 0.024 mmol) under N₂ atmosphere at 0° C. and the mixture was stirred at 120° C. for 3 hours. The mixture was diluted with EtOAc, washed with water and brine, dried over anhydrous Na₂SO₄, filtered, and concentrated under reduced pressure to dryness. The residue was purified by prep-TLC (PE:EtOAc=5:1) to give methyl 2-(6-oxo-2-phenyl-5-(4,5,6,7-tetrahydrobenzo[d]thiazol-2-yl)pyrimidin-1(6H)-yl)acetate (30 mg, yield 33.0%) as a yellow solid. ¹H NMR (400 MHz, CD₃OD) δ 9.37 (s, 1H), 8.38 (dd, J=7.7, 1.8 Hz, 2H), 7.51 (t, J=6.3 Hz, 3H), 5.28 (s, 2H), 3.80 (s, 3H), 2.88 (dd, J=13.5, 5.7 Hz, 4H), 1.98-1.90 (m, 4H). LC/MS (ESI) m/z: 382 (M+H)⁺.

### Step 4: 2-(6-Oxo-2-phenyl-5-(4,5,6,7-tetrahydrobenzo[d]thiazol-2-yl)pyrimidin-1(6H)-yl)acetic acid (5)

To a solution of methyl 2-(6-oxo-2-phenyl-5-(4,5,6,7-tetrahydrobenzo[d]thiazol-2-yl)pyrimidin-1(6H)-yl)acetate (30 mg, 0.079 mmol) in MeOH (2 mL) and THF (1 mL) was added a solution of LiOH·H₂O (13 mg, 0.31 mmol) in water (1 mL) and the mixture was stirred at room temperature for 1 hour. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give 2-(6-oxo-2-phenyl-5-(4,5,6,7-tetrahydrobenzo[d]thiazol-2-yl)pyrimidin-1(6H)-yl)acetic acid (20 mg, yield 69.2%) as a white solid, which was used directly in the next reaction without further purification. LC/MS (ESI) m/z: 368 (M+H)⁺.

### Step 5: N-((1H-pyrrolo[3,2-c]pyridin-2-yl)methyl)-2-(6-oxo-2-phenyl-5-(4,5,6,7-tetrahydrobenzo[d]thiazol-2-yl)pyrimidin-1(6H)-yl)acetamide (Compound 30)

To a mixture of 2-(6-oxo-2-phenyl-5-(4,5,6,7-tetrahydrobenzo[d]thiazol-2-yl) pyrimidin-1(6H)-yl)acetic acid (20 mg, 0.054 mmol) and ((1H-pyrrolo[3,2-c]pyridin-2-yl)methanamine (12 mg, 0.082 mmol) in DMF (3 mL) was added DIPEA (31 mg, 0.27 mmol) and HBTU (35 mg, 0.082 mmol) under N₂ atmosphere and the reaction mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 30 (1.4 mg, yield 5.2%) as a white solid. ¹H NMR (400 MHz, CD₃OD) δ 9.34 (s, 1H), 8.48 (s, 1H), 8.31-8.28 (m, 2H), 8.05 (d, J=6.1 Hz, 1H), 7.42-7.37 (m, 1H), 7.33-7.29 (m, 3H), 6.53 (s, 1H), 5.23 (s, 2H), 4.64 (s, 2H), 2.85-2.80 (m, 4H), 1.94-1.87 (m, 4H). LC/MS (ESI) m/z: 497 (M+H)⁺. RT (Method A): 1.53 min.

### Scheme 11. Synthesis of N-(2-((1-(2-(((1H-pyrrolo[3,2-c]pyridin-2-yl)methyl)amino)-2-oxoethyl)-6-oxo-2-phenyl-1,6-dihydropyrimidin-5-yl)amino)ethyl)-4-phenoxybenzamide (Compound 33)

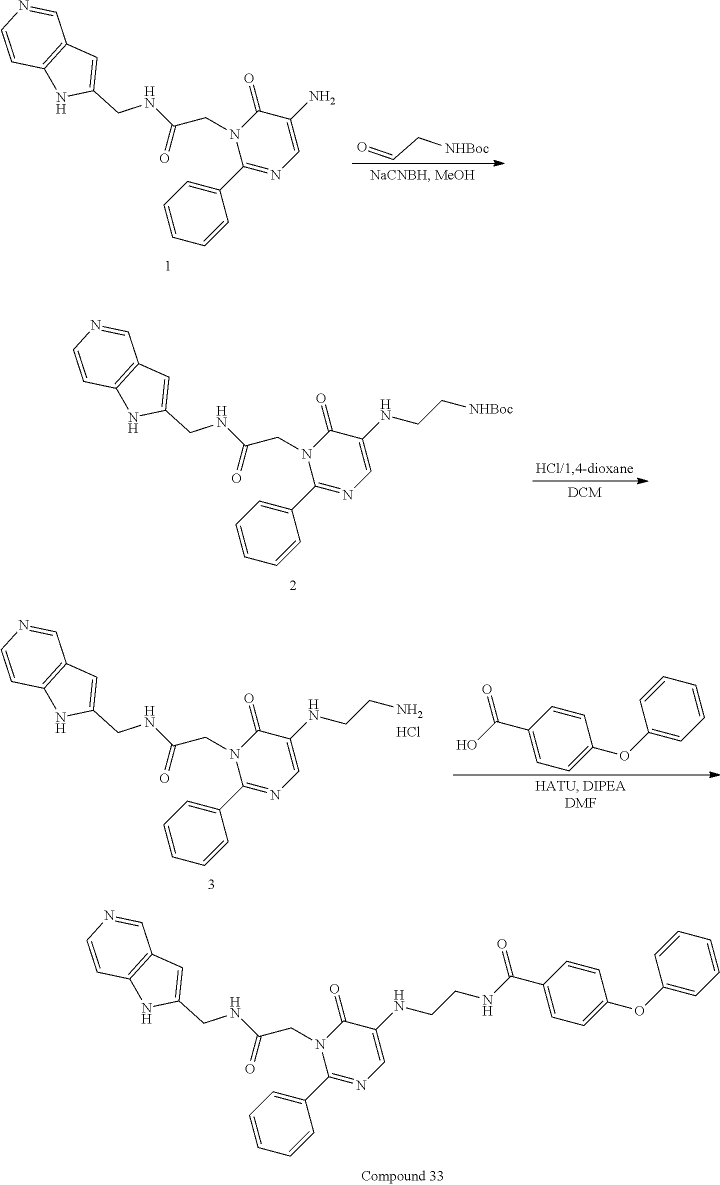

### Step 1: Tert-butyl (2-((1-(2-(((1H-pyrrolo[3,2-c]pyridin-2-yl)methyl)amino)-2-oxoethyl)-6-oxo-2-phenyl-1,6-dihydropyrimidin-5-yl)amino)ethyl)carbamate (2)

To a mixture of tert-butyl (2-oxoethyl)carbamate (85 mg, 0.52 mmol) and N-((1H-pyrrolo[3,2-c]pyridin-2-yl)methyl)-2-(5-amino-6-oxo-2-phenylpyrimidin-1(6H)-yl)acetamide (50 mg, 0.13 mmol) in MeOH (2 mL) was added NaBH₃CN (74 mg, 1.14 mmol) at room temperature and the reaction mixture was stirred at 50° C. for 2 hours. The mixture was diluted with EtOAc, washed with saturated aq. NH₄Cl solution and brine, dried over anhydrous Na₂SO₄, filtered, and concentrated under reduced pressure to dryness to give tert-butyl (2-((1-(2-(((1H-pyrrolo[3,2-c]pyridin-2-yl)methyl)amino)-2-oxoethyl)-6-oxo-2-phenyl-1,6-dihydropyrimidin-5-yl)amino)ethyl)carbamate (60 mg, yield 87.0%) as a white solid. LC/MS (ESI) m/z: 518 (M+H)⁺.

### Step 2: N-((1H-pyrrolo[3,2-c]pyridin-2-yl)methyl)-2-(5-((2-aminoethyl)amino)-6-oxo-2-phenylpyrimidin-1(6H)-yl)acetamide hydrochloride (3)

To a solution of tert-butyl (2-((1-(2-(((1H-pyrrolo[3,2-c]pyridin-2-yl)methyl)amino)-2-oxoethyl)-6-oxo-2-phenyl-1,6-dihydropyrimidin-5-yl)amino)ethyl)carbamate (60 mg, 0.12 mmol) in DCM (1 mL) was added HCl/1,4-dioxane (2 mL, 4M) and the mixture was stirred at room temperature for 2 hours. The reaction mixture was concentrated under reduced pressure to dryness to give N-((1H-pyrrolo[3,2-c]pyridin-2-yl)methyl)-2-(5-((2-aminoethyl)amino)-6-oxo-2-phenylpyrimidin-1(6H)-yl)acetamide hydrochloride (50 mg, crude) as a colorless oil, which was used directly in the next step without further purification. LC/MS (ESI) m/z: 418 (M+H)⁺.

### Step 3: N-(2-((1-(2-(((1H-pyrrolo[3,2-c]pyridin-2-yl)methyl)amino)-2-oxoethyl)-6-oxo-2-phenyl-1,6-dihydropyrimidin-5-yl)amino)ethyl)-4-phenoxybenzamide (Compound 33)

To a mixture of 4-phenoxybenzoic acid (24 mg, 0.11 mmol) and N-((1H-pyrrolo[3,2-c]pyridin-2-yl)methyl)-2-(5-((2-aminoethyl)amino)-6-oxo-2-phenylpyrimidin-1(6H)-yl)acetamide hydrochloride (50 mg, 0.11 mmol) in DMF (2 mL) was added DIPEA (87 mg, 0.66 mmol) and HATU (50 mg, 0.13 mmol) under N₂ atmosphere and the reaction mixture was stirred at room temperature for 2 hours. The mixture was concentrated under reduced pressure to dryness. The residue was purified by prep-TLC (DCM:MeOH=7:1) and further purified by prep-HPLC to give Compound 33 (2 mg, yield 3.0%) as a white solid. ¹H NMR (400 MHz, CD₃OD) δ 8.75 (d, J=0.8 Hz, 1H), 8.12 (d, J=6.0 Hz, 1H), 7.85-7.77 (m, 2H), 7.52-7.43 (m, 4H), 7.39 (dd, J=11.2, 4.5 Hz, 4H), 7.29 (s, 1H), 7.19 (t, J=7.4 Hz, 1H), 7.04 (dd, J=8.6, 1.0 Hz, 2H), 6.99-6.95 (m, 2H), 6.53 (d, J=8.3 Hz, 1H), 4.62 (s, 2H), 4.55 (s, 2H), 3.66 (t, J=6.1 Hz, 2H), 3.43 (t, J=6.1 Hz, 2H). LC/MS (ESI) m/z: 614 (M+H)⁺. RT (Method A): 1.38 min.

### Scheme 12. Synthesis of N-(2-((1-(2-(((1H-pyrrolo[3,2-c]pyridin-2-yl)methyl)amino)-2-oxoethyl)-6-oxo-2-phenyl-1,6-dihydropyrimidin-5-yl)amino)ethyl)dibenzo[b,d]furan-2-carboxamide (Compound 35)

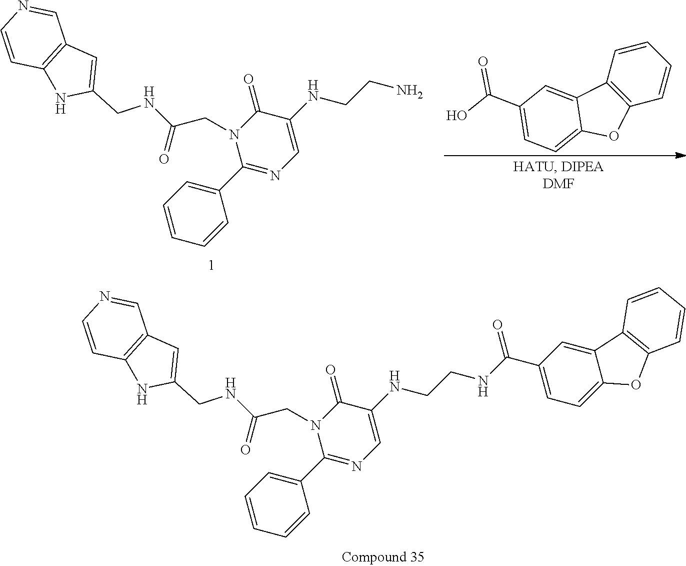 [SMILES: NCCNc1cnc(-c2ccccc2)n(CC(=O)NCc2cc3cnccc3[nH]2)c1=O ; O=C(O)c1ccc2oc3ccccc3c2c1 ; O=C(Cn1c(-c2ccccc2)ncc(NCCNC(=O)c2ccc3oc4ccccc4c3c2)c1=O)NCc1cc2cnccc2[nH]1]

To a mixture of dibenzo[b,d]furan-2-carboxylic acid (20 mg, 0.09 mmol) and N-((1H-pyrrolo[3,2-c]pyridin-2-yl)methyl)-2-(5-((2-aminoethyl)amino)-6-oxo-2-phenylpyrimidin-1(6H)-yl)acetamide (50 mg, 0.12 mmol) in DMF (2 mL) was added DIPEA (73 mg, 0.54 mmol) and HATU (36 mg, 0.09 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=10:1) and further purified by prep-HPLC to give Compound 35 (11 mg, yield 19.1%) as a white solid. ¹H NMR (400 MHz, CD₃OD) δ 8.72 (s, 1H), 8.53 (d, J=1.5 Hz, 1H), 8.10 (d, J=6.1 Hz, 1H), 8.05 (d, J=7.2 Hz, 1H), 8.00 (dd, J=8.7, 1.9 Hz, 1H), 7.63 (d, J=3.2 Hz, 1H), 7.61 (d, J=2.8 Hz, 1H), 7.54 (d, J=7.2 Hz, 1H), 7.49-7.45 (m, 4H), 7.38 (dd, J=8.9, 6.0 Hz, 3H), 7.34 (s, 1H), 6.56 (s, 1H), 4.63 (s, 2H), 4.57 (s, 2H), 3.74 (t, J=5.9 Hz, 2H), 3.51 (t, J=6.1 Hz, 2H). LC/MS (ESI) m/z: 612 (M+H)⁺. RT (Method A): 1.33 min.

Compound 36 was prepared based on Scheme 12:

| # | Reactant A | Reactant B | Characterization Data |
|---|---|---|---|
| 36 | 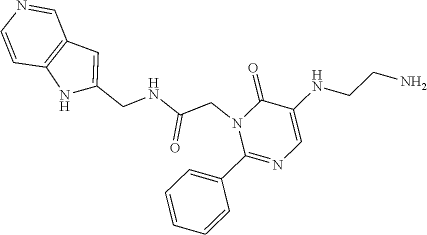 [SMILES: NCCNc1cnc(-c2ccccc2)n(CC(=O)NCc2cc3cnccc3[nH]2)c1=O] | 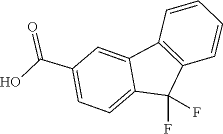 [SMILES: O=C(O)c1ccc2c(c1)-c1ccccc1C2(F)F] | ¹H NMR (400 MHz, CD₃OD) δ 8.72 (s, 1H), 8.14-8.07 (m, 2H), 7.87-7.81 (m, 1H), 7.74 (d, J = 7.6 Hz, 1H), 7.68 (d, J = 7.6 Hz, 1H), 7.63 (d, J = 7.6 Hz, 1H), 7.51 (t, J = 7.7 Hz, 1H), 7.44 (t, J = 6.3 Hz, 4H), 7.39 (dd, J = 14.0, 7.4 Hz, 3H),7.32 (s, 1H), 6.52 (s, 1H), 4.63 (s, 2H), 4.55 (s, 2H), 3.71 (t, J = 6.0 Hz, 2H), 3.49 (t, J = 5.9 Hz, 2H). LC/MS (ESI) m/z: 646 (M + H)⁺. RT (Method A): 1.45 min. |

### Scheme 13. Synthesis of (S)-7-((4-(4-fluorophenoxy)benzoyl)glycyl)-N—((S)-2-hydroxy-1-(1H-pyrrolo[3,2-c]pyridin-2-yl)ethyl)-1,4-dioxa-7-azaspiro[4.4]nonane-8-carboxamide (Compound 42)

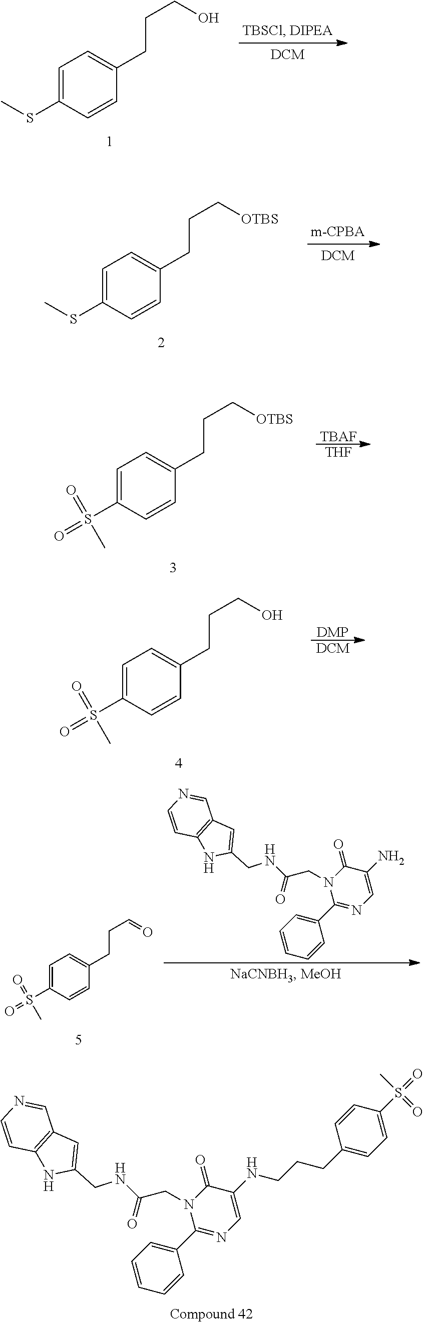

### Step 1: Tert-butyldimethyl(3-(4-(methylthio)phenyl)propoxy)silane (2)

To a solution of 3-(4-(methylthio)phenyl)propan-1-ol (260 mg, 1.43 mmol) in DCM (3 mL) was added TBSCl (323 g, 2.14 mmol) and TEA (554 mg, 4.29 mmol) successively at 0° C. and the mixture was stirred at room temperature for 3 hours. The mixture was diluted with water and extracted with DCM twice. The combined organic layers were washed with brine, dried over anhydrous Na₂SO₄, filtered, and concentrated to dryness. The residue was purified by flash chromatography on silica gel (0-20% EtOAc in PE) to give tert-butyldimethyl(3-(4-(methylthio)phenyl)propoxy)silane (330 mg, yield 78.0%) as a colorless oil.

### Step 2: Tert-butyldimethyl(3-(4-(methylsulfonyl)phenyl)propoxy)silane (3)

To a solution of tert-butyldimethyl(3-(4-(methylthio)phenyl)propoxy)silane (100 mg, 0.34 mmol) in DCM (2 mL) was added m-CPBA (233 mg, 1.35 mmol) under N₂ atmosphere and the reaction mixture was stirred at room temperature for 2 hours. The mixture was diluted with DCM, quenched with saturated aq. Na₂S₂O₃ solution, wash with saturated aq. NaHCO₃ solution and brine, dried over anhydrous Na₂SO₄, filtered, and concentrated under reduced pressure to dryness. The residue was purified by flash chromatography (silica gel, 0-50% EtOAc in PE) to give tert-butyldimethyl(3-(4-(methylsulfonyl)phenyl)propoxy)silane (100 mg, yield 89.7%) as a white solid.

### Step 3: 3-(4-(Methylsulfonyl)phenyl)propan-1-ol (4)

To a solution of tert-butyldimethyl(3-(4-(methylsulfonyl)phenyl)propoxy)silane (100 mg, 0.31 mmol) in THF (2 mL) was added TBAF (1 mL, 1 M in THF) and the mixture was stirred under N₂ atmosphere at room temperature for 2 hours. The reaction mixture was concentrated under reduced pressure to dryness. The residue was purified by prep-TLC (PE:EtOAc=1:2) to give 3-(4-(methylsulfonyl)phenyl)propan-1-ol (58 mg, yield 87.9%) as a colorless oil. ¹H NMR (400 MHz, CDCl₃) δ 7.80 (s, 1H), 7.78 (s, 1H), 7.34 (d, J=7.9 Hz, 2H), 3.62 (t, J=6.2 Hz, 2H), 2.98 (s, 3H), 2.75 (t, J=7.7 Hz, 2H), 1.88-1.81 (m, 2H).

### Step 4: 3-(4-(Methylsulfonyl)phenyl)propanal (5)

To a solution of 3-(4-(methylsulfonyl)phenyl)propan-1-ol (58 mg, 0.27 mmol) in DCM (2 mL) was added Dess Martin (172 mg, 0.41 mmol) under N₂ atmosphere and the reaction mixture was stirred at room temperature for 2 hours. The mixture was diluted with DCM, quenched with saturated aq. Na₂S₂O₃ solution, wash with saturated aq. NaHCO₃ solution and brine, dried over anhydrous Na₂SO₄, filtered, and concentrated under reduced pressure to dryness. The residue was purified by prep-TLC (DCM:MeOH=20:1) to give 3-(4-(methylsulfonyl)phenyl)propanal (55 mg, yield 94.5%) as a white solid.

### Step 5: N-((1H-pyrrolo[3,2-c]pyridin-2-yl)methyl)-2-(5-((3-(4-(methylsulfonyl)phenyl) propyl)amino)-6-oxo-2-phenylpyrimidin-1(6H)-yl)acetamide (Compound 42)

To a solution of N-((1H-pyrrolo[3,2-c]pyridin-2-yl)methyl)-2-(5-amino-6-oxo-2-phenylpyrimidin-1(6H)-yl)acetamide (34 mg, 0.090 mmol) in MeOH (1 mL) was added 3-(4-(methylsulfonyl)phenyl)propanal (57 mg, 0.27 mmol) and NaBH₃CN (23 mg, 0.36 mmol) under N₂ atmosphere and the reaction mixture was stirred at 50° C. for 2 hours. The mixture was concentrated under reduced pressure to dryness and the residue was purified by prep-HPLC to give Compound 42 (1.5 mg, yield 2.9%) as a white solid. ¹H NMR (400 MHz, CD₃OD) δ 8.74 (s, 1H), 8.13 (d, J=5.9 Hz, 1H), 7.88 (d, J=8.3 Hz, 2H), 7.46 (ddd, J=20.6, 15.3, 7.7 Hz, 8H), 7.11 (s, 1H), 6.52 (s, 1H), 4.62 (s, 2H), 4.55 (s, 2H), 3.23 (t, J=6.9 Hz, 2H), 3.10 (s, 3H), 2.92-2.85 (m, 2H), 2.09-2.00 (m, 2H). LC/MS (ESI) m/z: 571 (M+H)⁺.

### Scheme 14. Synthesis of N-((1H-pyrrolo[3,2-c]pyridin-2-yl)methyl)-2-(5-((3-(4-methoxyphenyl)propyl)amino)-6-oxo-2-phenylpyrimidin-1(6H)-yl)acetamide (Compound 45)

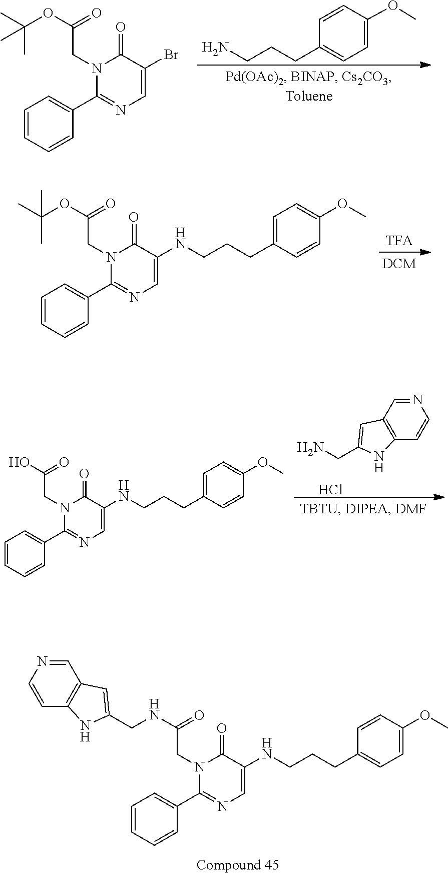

### Step 1: tert-butyl 2-(5-((3-(4-methoxyphenyl)propyl)amino)-6-oxo-2-phenylpyrimidin-1(6H)-yl)acetate

A vial was charged with tert-butyl 2-(5-bromo-6-oxo-2-phenyl-pyrimidin-1-yl)acetate (0.05 g, 0.1369 mmol), 3-(4-methoxyphenyl)propan-1-amine (0.023 g, 0.1369 mmol), cesium carbonate (0.0893 g, 0.2738 mmol), BINAP (0.01705 g, 0.02738 mmol) and palladium acetate (0.003074 g, 0.01369 mmol). Toluene was added in it and the reaction mixture was purged with nitrogen for 3 minutes. The reaction mixture was allowed to stir at 120° C. for 10 minutes under microwave radiation. The crude mixture was purified using flash chromatography (silica gel, 0-50% EtOAc in heptane) to give tert-butyl 2-(5-((3-(4-methoxyphenyl)propyl)amino)-6-oxo-2-phenylpyrimidin-1(6H)-yl)acetate (0.02 g, 37% yield). LC/MS (ESI) m/z: 450 (M+H)⁺.

### Step 2: 2-(5-((3-(4-methoxyphenyl)propyl)amino)-6-oxo-2-phenylpyrimidin-1(6H)-yl)acetic acid

tert-butyl 2-(5-((3-(4-methoxyphenyl)propyl)amino)-6-oxo-2-phenylpyrimidin-1(6H)-yl)acetate (0.02 g, 0.054 mmol) was dissolved DCM (3 mL). TFA (3 ML) was added in it and the reaction was allowed to stir at rt for 1 hr. Excess TFA was evaporated, and 1 M HCl in MeOH was added into the crude mass and allowed to stir for 5 minutes. Methanol was evaporated to dryness to give 2-(5-((3-(4-methoxyphenyl)propyl)amino)-6-oxo-2-phenylpyrimidin-1(6H)-yl)acetic acid (0.020 g, 96% yield) as a brown solid which was used in the next step without further purification. LC/MS (ESI) m/z: 394 (M+H)⁺.

### Step 3: N-((1H-pyrrolo[3,2-c]pyridin-2-yl)methyl)-2-(5-((3-(4-methoxyphenyl)propyl)amino)-6-oxo-2-phenylpyrimidin-1(6H)-yl)acetamide (Compound 45)

2-[5-[3-(4-methoxyphenyl)propylamino]-6-oxo-2-phenyl-pyrimidin-1-yl]acetic acid (0.015 g, 0.038 mmol), 1H-pyrrolo[3,2-c]pyridin-2-ylmethanamine hydrochloride (0.0070 g, 0.038 mmol), TBTU (0.025 g, 0.076 mmol), and DIPEA (0.14 mL, 0.076 mmol) were stirred in DMF (5 ml) at 3 for 30 minute. Water (30 mL) was added into the reaction mixture and the solid was filtered. The solid was dissolved in DCM and dried over anhydrous Na₂SO₄, filtered, and concentrated to dryness. The residue was purified by flash chromatography (silica gel, 0-5% MeOH in DCM) to give 2-[5-[3-(4-methoxyphenyl)propylamino]-6-oxo-2-phenyl-pyrimidin-1-yl]-N-(1H-pyrrolo[3,2-c]pyridin-2-ylmethyl)acetamide as a pale white solid (0.007 g, 40%) yield). 1H NMR (400 MHz, MeOD-CDCl₃) 8.71 (s, 1 NH), 8.12 (d, J=5.8 Hz, 1H), 7.51-7.42 (d, 3H), 7.38 (dd, J=11.9, 6.7 Hz, 3H), 7.17-7.07 (m, 3H), 6.89-6.81 (s, 2H), 6.47 (s, 1H), 4.61 (s, 2H), 4.56 (s, 2H), 3.79 (s, 3H), 3.16 (t, J=7.0 Hz, 2H), 2.71 (t, J=7.5 Hz, 2H), 2.05-1.93 (m, 2H), 1.28 (s, 13H). LC/MS (ESI) m/z: 523 (M+H)⁺. RT (Method A): 1.45 min.

The following compounds were prepared based on Scheme 14:

| # | Reactant A | Reactant B | Reactant C | Characterization Data |
|---|---|---|---|---|
| 27 | 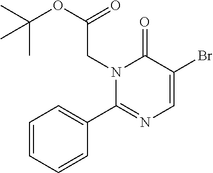 [SMILES: CC(C)(C)OC(=O)Cn1c(-c2ccccc2)ncc(Br)c1=O] | 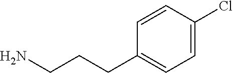 [SMILES: NCCCc1ccc(Cl)cc1] | 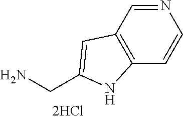 [SMILES: NCc1cc2cnccc2[nH]1] | ¹H NMR (400 MHz, MeOD- CDCl₃) δ 8.74 (dt, J = 3.6, 1.7 Hz, 1H), 8.13 (d, J = 4.5 Hz, 1H), 7.71 (s, 1H), 7.56- 7.45 (m, 3H), 7.42 (dd, J = 12.8, 10.1 Hz, 3H), 7.27 (d, J = 8.8 Hz, 2H), 7.19 (d, J = 8.9 Hz, 2H), 7.11 (s, 1H), 6.51 (s, 1H), 4.59 (d, J = 20.9 Hz, 4H), 3.23-3.14 (m, 2H), 2.80-2.71 |
| | | | | (m, 2H), 2.07-1.92 (m, 2H). |
| | | | | LC/MS (ESI) m/z: 527 (M + |
| | | | | H)⁺. RT (Method A): 1.70 |
| | | | | min. |
| 28 | 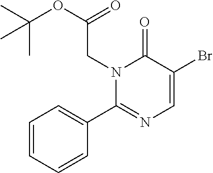 | 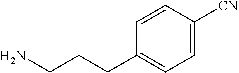 [SMILES: N#Cc1ccc(CCCN)cc1] | 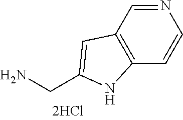 [SMILES: NCc1cc2cnccc2[nH]1] | ¹H NMR (400 MHZ, CDCl₃- MeOD) δ 8.71 (s, 1H), 8.13 (d, J = 6.6 Hz, 1H), 7.64 (d, J = 8.6 Hz, 2H), 7.55-7.37 (m, 7H), 7.34 (q, J = 4.7 Hz, 2H), 7.19 (s, 1H), 6.46 (s, 1H), 4.59 (s, 2H), 4.55 (s, 2H), 3.52-3.43 (m, 2H), 3.11-3.02 (m, 2H). LC/MS (ESI) m/z: 504 (M + H)⁺. RT (Method A): 1.17 min. |
| 46 | 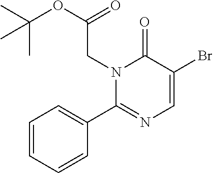 | 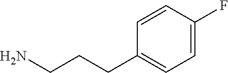 [SMILES: NCCCc1ccc(F)cc1] | 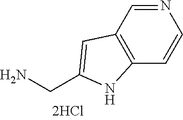 [SMILES: NCc1cc2cnccc2[nH]1] | ¹H NMR (400 MHz, MeOD- CDCl₃) δ 8.72 (s, 1H), 8.12 (d, J = 5.9 Hz, 1H), 7.52-7.44 (m, 3H), 7.39 (t, J = 7.1 Hz, 3H), 7.25-7.16 (m, 2H), 7.10 (s, 1H), 7.04-6.94 (m, 2H), 6.49 (s, 1H), 4.62 (s, 2H), 4.56 (s, 2H), 3.18 (t, J = 6.9 Hz, 2H), 2.75 (t, J = 7.6 Hz, 2H), 2.01 (q, J = 7.2 Hz, 2H). LC/MS (ESI) m/z: 511 (M + |
| | | | | H)⁺. RT (Method A): |
| | | | | 1.56 min. |
| 212^{a} | 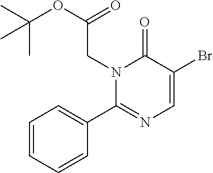 | 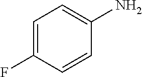 | 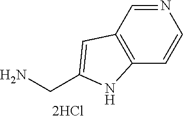 [SMILES: NCc1cc2cnccc2[nH]1] | ¹H-NMR (400 MHz, DMSO- d6) δ 11.42 (s, 1H), 8.89-8.63 (m, 2H), 8.40 (s, 1H), 8.11 (s, 1H), 7.80 (dd, J = 8.9, 3.8 Hz, 2H), 7.55-7.25 (m,7 H), 7.19- 7.07 (m, 2H), 6.32 (s, 1H), 4.56 (s, 2H), 4.44 (d, J = 5.8 Hz, 2H). LC/MS (ESI) m/z: 469 (M + H)⁺. RT (Method A): 1.20 min. |
| 224^{b} | 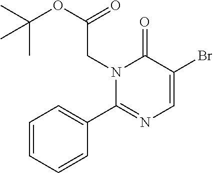 | 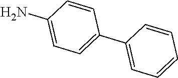 [SMILES: Nc1ccc(-c2ccccc2)cc1] | 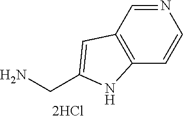 [SMILES: NCc1cc2cnccc2[nH]1] | ¹H-NMR (400 MHz, DMSO- d6) δ 11.34 (s, 2H), 8.75 (s, 2H), 8.49 (s, 1H), 8.23 (s, 1H), 7.97 (d, J = 8.1 Hz, 2H), 7.68-7.24 (m, 12H), 6.34 (s, 1H), 4.62-4.37 (m, 4H). LC/MS (ESI) m/z = 527 (M + H)⁺. RT (Method A): 1.70 min. |
| 228ª | 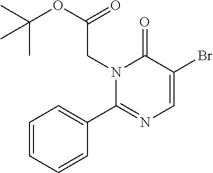 [SMILES: CC(C)(C)OC(=O)Cn1c(-c2ccccc2)ncc(Br)c1=O] | 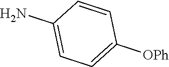 [SMILES: Nc1ccc(Oc2ccccc2)cc1] | 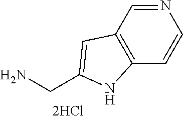 [SMILES: NCc1cc2cnccc2[nH]1] | ¹H-NMR (400 MHz, MeOD) δ 8.88 (s, 1H), 8.48 (s, 1H), 8.21 (d, J = 6.2 Hz, 1H), 7.77 (s, 1H), 7.64 (d, J = 6.2 Hz, 1H), 7.57-7.40 (m, 5H), 7.38-7.31 (m, 2H), 7.31-7.23 (m, 2H), 7.09 (t, J = 7.4 Hz, 1H), 7.10-6.95 (m, 2H), 6.70 (s, 1H), 4.70 (s, 2H), 4.61 (s, 2H), 4.54 (s, 2H). LC/MS |
| | | | | (ESI) m/z: 543 (M + H)⁺. |
| | | | | RT (Method A): |
| | | | | RT (Method A): 1.60 min. |
| 234^{c} | 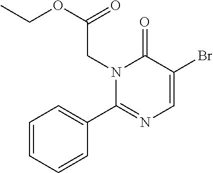 | 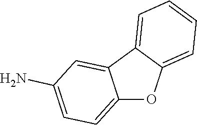 [SMILES: Nc1ccc2oc3ccccc3c2c1] | 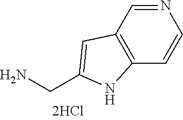 [SMILES: NCc1cc2cnccc2[nH]1] | ¹H-NMR (400 MHz, DMSO- d6) δ 8.95-8.80 (m, 1H), 8.24- 7.99 (m, 4H), 7.95-7.80 (m, 4H), 7.66 (t, J = 8.0 Hz, 1H), 7.55-7.30 (m, 9H), 4.68-4.40 (m, 4H). LC/MS (ESI) m/z: 541 (M + H)⁺. RT (Method A): 1.62 min. |
| 236ª | 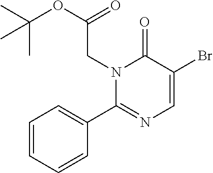 | 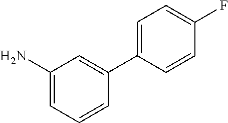 [SMILES: Nc1cccc(-c2ccc(F)cc2)c1] | 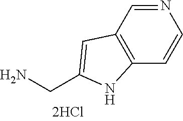 [SMILES: NCc1cc2cnccc2[nH]1] | ¹H-NMR (400 MHz, DMSO- d6) δ 11.27 (s, 1H), 8.67 (s, 2H), 8.27 (s, 1H), 8.12-7.74 (m, 3H), 7.62 (s, 2H), 7.51- 7.04 (m, 10H), 6.26 (s, 1H), 4.51 (s, 2H), 4.37 (d, J = 5.5 Hz, 2H). LC/MS (ESI) m/z: 545 (M + H)⁺. RT (Method A): 1.72 min. |
| 244^{a} | 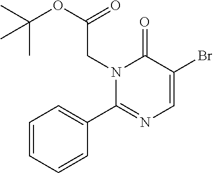 | 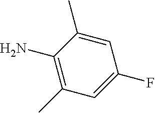 | 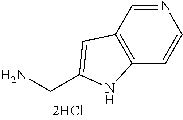 [SMILES: NCc1cc2cnccc2[nH]1] | ¹H-NMR (400 MHz, DMSO- d6) δ 11.38 (s, 1H), 8.74 (d, J = 5.8 Hz, 2H), 8.32 (s, 1H), 8.12 (d, J = 5.7 Hz, 1H), 7.53- 7.22 (m, 5H), 7.18-6.90 (m, 2H), 6.49 (s, 1H), 6.34 (s, 1H), 4.64-4.28 (m, 4H), 2.18 (d, J = 6.2 Hz, 6H). LC/MS (ESI) m/z: 497 (M + H)⁺. RT (Method A): 1.42 min. |
| 245ª | 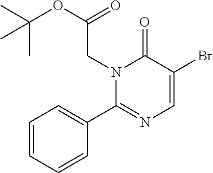 | 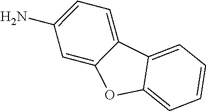 [SMILES: Nc1ccc2c(c1)oc1ccccc12] | 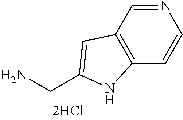 [SMILES: NCc1cc2cnccc2[nH]1] | ¹H-NMR (400 MHz, DMSO- d6) δ 11.35 (s, 1H), 8.75 (d, J = 6.1 Hz, 2H), 8.27-8.04 (m, 5H), 8.00 (dd, J = 8.1, 6.0 Hz, 2H), 7.62 (d, J = 8.1 Hz, 1H), 7.57-7.21 (m, 8H), 6.34 (s, 1H), 4.59 (s, 2H), 4.44 (d, J = 5.6 Hz, 2H). LC/MS (ESI) m/z: 541 (M + H)⁺. RT (Method A): |
| | | | | 1.65 min. |
| 307^{d} | 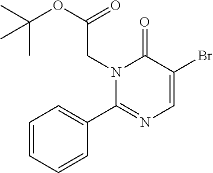 [SMILES: CC(C)(C)OC(=O)Cn1c(-c2ccccc2)ncc(Br)c1=O] | 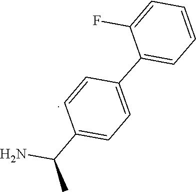 [SMILES: C[C@@H](N)c1ccc(-c2ccccc2F)cc1] | 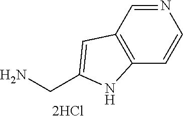 [SMILES: NCc1cc2cnccc2[nH]1] | ¹H NMR (400 MHz, CD₃OD) δ 8.71 (s, 1H), 8.11 (d, J = 5.6 Hz, 1H), 7.55-7.45 (m, 5H), 7.41 (t, J = 8.5 Hz, 4H), 7.35 (d, J = 7.1 Hz, 3H), 7.23 (t, J = 7.5 Hz, 1H), 7.19-7.14 (m, 1H), 6.90 (s, 1H), 6.48 (s, 1H), 4.63 (s, 2H), 4.57 (d, J = 6.6 Hz, 1H), 4.55 (s, 2H), 1.63 (d, J = 6.5 Hz, 3H). LC/MS (ESI) |
| | | | | m/z: 573 (M + H)⁺. |
| | | | | RT (Method A): 1.72 |
| | | | | min. |
| 454^{e} | 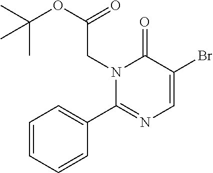 | 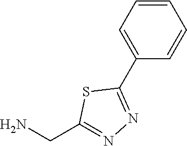 [SMILES: NCc1nnc(-c2ccccc2)s1] | 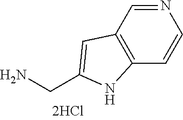 [SMILES: NCc1cc2cnccc2[nH]1] | ¹H NMR (400 MHz, MeOD- CDCl3) δ 8.79 (s, 1H), 8.17 (s, 1H), 7.95 (t, J = 7.6 Hz, 1H), 7.60- 7.36 (m, 7H), 7.36-7.28 (m, 2H), 7.24 (dd, J = 11.1, 8.4 Hz, 1H), 6.81 (d, J = 3.7 Hz, 1H), 6.61 (s, 1H), 4.68-4.50 (m, 6H). LC/MS (ESI) m/z: 550 (M + H)⁺. |
| | | | | RT (Method A): |
| | | | | 1.10 min. |
| 455^{e} | 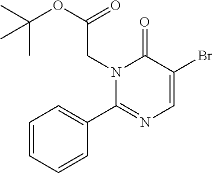 | 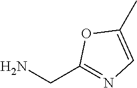 | 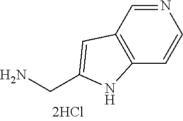 [SMILES: NCc1cc2cnccc2[nH]1] | ¹H NMR (400 MHz, MeOD- CDCl3) δ 7.65 (s, 1H), 7.59-7.44 (m, 4H), 7.39 (t, J = 7.4 Hz, 3H), 7.28 (s, 1H), 6.72 (s, 1H), 6.50 (s, 1H), 4.59 (d, J = 12.8 Hz, 4H), 4.49 (s, 2H), 2.33 (s, 3H). LC/MS (ESI) m/z: 470 (M + H)⁺. RT (Method A): 0.70 min. |
| 456^{e} | 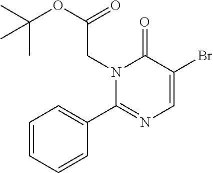 | 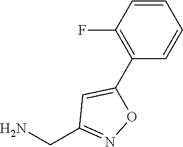 [SMILES: NCc1cc(-c2ccccc2F)on1] | 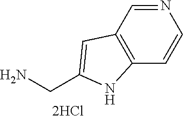 [SMILES: NCc1cc2cnccc2[nH]1] | ¹H NMR (400 MHz, MeOD- CDCl₃) δ 8.79 (s, 1H), 8.17 (s, 1H), 7.95 (t, J = 7.6 Hz, 1H), 7.60-7.36 (m, 7H), 7.36-7.28 (m, 2H), 7.24 (dd, J = 11.1, 8.4 Hz, 1H), 6.81 (d, J = 3.7 Hz, 1H), 6.61 (s, 1H), 4.68- 4.50 (m, 6H). LC/MS (ESI) m/z: 550 (M + H)⁺. RT (Method A): 1.35 min. |
| 457^{e} | 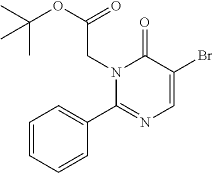 | 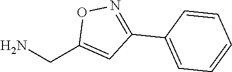 [SMILES: NCc1cc(-c2ccccc2)no1] | 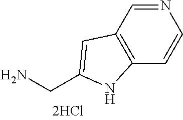 [SMILES: NCc1cc2cnccc2[nH]1] | ¹H NMR (400 MHz, MeOD- CDCl₃) δ 8.72 (s, 1H), 8.13 (d, J = 5.9 Hz, 1H), 7.80 (dd, J = 6.7, 3.0 Hz, 2H), 7.67 (s, 1H), 7.47 (td, J = 8.2, 5.5 Hz, 6H), 7.40 (d, J = 6.8 Hz, 3H), 7.26 (s, 1H), 6.73 (s, 1H), 6.49 (s, 1H), 4.69-4.53 (m, 6H). LC/MS (ESI) m/z: 532 (M + H)⁺. RT (Method A): 1.23 min. |
| 458^{e} | 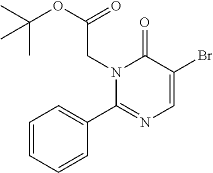 | 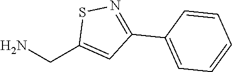 [SMILES: NCc1cc(-c2ccccc2)ns1] | 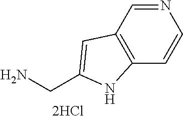 [SMILES: NCc1cc2cnccc2[nH]1] | ¹H NMR (400 MHz, MeOD- CDCl₃) δ 8.72 (s, 1H), 8.12 (d, J = 5.9 Hz, 1H), 7.98-7.88 (m, 2H), 7.69 (s, 1H), 7.56- 7.33 (m, 9H), 7.22 (s, 1H), 6.49 (s, 1H), 4.81 (s, 2H), 4.64 (s, 2H), 4.58 (s, 2H). LC/MS (ESI) m/z: 548 (M + H)⁺. RT (Method A): 1.32 min. |
| 459^{e} | 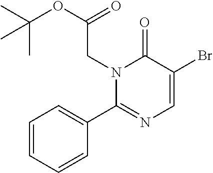 | 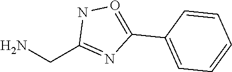 | 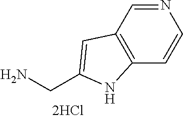 [SMILES: NCc1cc2cnccc2[nH]1] | ¹H NMR (400 MHz, MeOD- CDCl₃) δ 8.74 (s, 1H), 8.13 (dd, J = 6.6, 2.5 Hz, 3H), 7.65 (d, J = 9.0 Hz, 1H), 7.57 (t, J = 7.6 Hz, 2H), 7.54-7.33 (m, 7H), 6.52 (s, 1H), 4.67-4.55 (m, 6H). LC/MS (ESI) m/z: 533 (M + H)⁺. RT (Method A): 1.20 min. |
| 464^{e} | 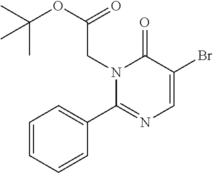 | 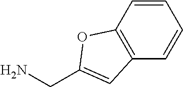 [SMILES: NCc1cc2ccccc2o1] | 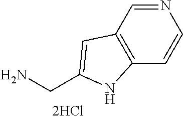 [SMILES: NCc1cc2cnccc2[nH]1] | ¹H NMR (400 MHz, MeOD- CDCl₃) δ 8.73 (s, 1H), 8.13 (d, J = 5.9 Hz, 1H), 7.59- 7.16 (m, 11H), 6.72 (s, 1H), 6.49 (s, 1H), 4.63-4.52 (m, 6H). LC/MS: (ESI) m/z = 505 [M + H]⁺. RT (Method A): 1.11 min. |
| 465^{e} | 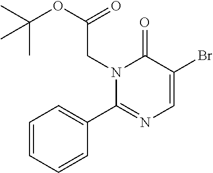 | 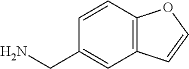 [SMILES: NCc1ccc2occc2c1] | 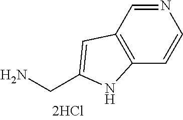 [SMILES: NCc1cc2cnccc2[nH]1] | ¹H NMR (400 MHz, MeOD- CDCl₃) δ 8.71 (s, 1H), 8.11 (d, J = 5.8 Hz, 1H), 7.73-7.58 (m, 2H), 7.53-7.41 (m, 4H), 7.41- 7.29 (m, 4H), 7.14 (s, 1H), 6.78 (d, J = 2.2 Hz, 1H), 6.47 (s, 1H), 4.59 (d, J = 14.7 Hz, 4H), 4.50 (s, 2H). LC-MS: (ESI+) m/z = 505 [M + H]+. RT (Method A): 1.22 min. |
| 472^{e} | 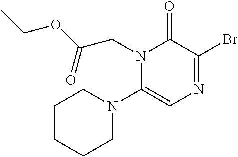 [SMILES: CCOC(=O)Cn1c(N2CCCCC2)cnc(Br)c1=O] | 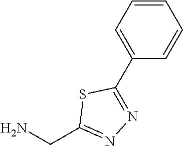 [SMILES: NCc1nnc(-c2ccccc2)s1] | 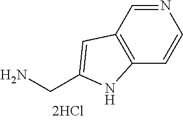 [SMILES: NCc1cc2cnccc2[nH]1] | ¹H NMR (400 MHz, CDCl₃) δ 8.59 (s, 1H), 8.13 (s, 2H), 7.92-7.77 (m, 2H), 7.45 (q, J = 7.1 Hz, 3H), 7.23 (d, J = 5.7 Hz, 1H), 7.06 (s, 1H), 6.78 (s, 1H), 6.31 (s, 1H), 4.92 (d, J = 5.9 Hz, 4H), 4.62 (d, J = 6.0 Hz, 2H), 3.13 (d, J = 11.2 Hz, 2H), 2.62 (t, J = 11.5 |
| | | | | Hz, 2H), 1.69 (dd, J = 87.1, |
| | | | | 12.8 Hz, 6H). LC/MS (ESI) |
| | | | | m/z: 557 (M + H)⁺. RT |
| | | | | (Method A): 1.40 min. |
| 473^{e} | 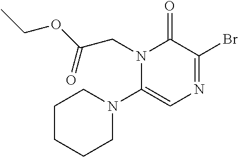 [SMILES: CCOC(=O)Cn1c(N2CCCCC2)cnc(Br)c1=O] | 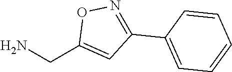 [SMILES: NCc1cc(-c2ccccc2)no1] | 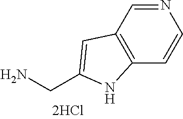 [SMILES: NCc1cc2cnccc2[nH]1] | ¹H NMR (400 MHz, DMSO) δ 11.38 (s, 1H), 8.75 (d, J = 5.4 Hz, 2H), 8.12 (d, J = 5.7 Hz, 1H), 7.84( dd, J = 6.6, 3.0 Hz, 2H), 7.62 (t, J = 6.2 Hz, 1H), 7.48 (p, J = 3.8 Hz, 3H), 7.36 (d, J = 5.7 Hz, 1H), 6.81 (s, 1H), 6.64 (s, 1H), 6.45 (s, 1H), 4.69 (s, 2H), 4.64 |
| | | | | (d, J = 6.1 Hz, 2H), 4.48 |
| | | | | (d, J = 5.6 Hz, 2H), 2.94 (d, |
| | | | | J = 11.1 Hz, 2H), 1.68- |
| | | | | 1.26 (m, 6H). LC/MS (ESI) |
| | | | | m/z: 539 (M + H)⁺. |
| | | | | RT (Method A): 1.45 min. |
| 474^{e} | 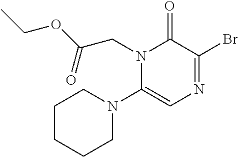 [SMILES: CCOC(=O)Cn1c(N2CCCCC2)cnc(Br)c1=O] | 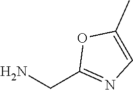 | 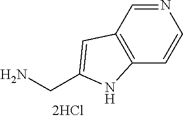 [SMILES: NCc1cc2cnccc2[nH]1] | ¹H NMR (400 MHz, DMSO) δ 11.48 (s, 1H), 8.79 (d, J = 6.6 Hz, 2H), 8.14 (d, J = 5.7 Hz, 1H), 7.50- 7.25 (m, 2H), 6.70 (d, J = 1.4 Hz, 1H), 6.61 (s, 1H), 6.48 (s, 1H), 4.68 (s, 2H), 4.49 (t, J = 5.7 Hz, 4H), 2.93 (d, J = 11.1 Hz, 2H), 2.46 (s, 2H), 2.24 (s, 3H), |
| | | | | 1.74-1.25 (m, 6H). LC/MS |
| | | | | (ESI) m/z: 477 (M + H)⁺. |
| | | | | RT (Method A): 1.03 min. |
| 477^{e} | 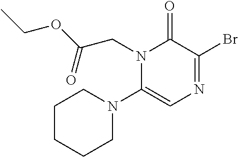 [SMILES: CCOC(=O)Cn1c(N2CCCCC2)cnc(Br)c1=O] | 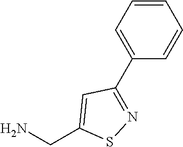 [SMILES: NCc1cc(-c2ccccc2)ns1] | 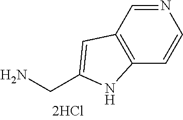 [SMILES: NCc1cc2cnccc2[nH]1] | ¹H NMR (400 MHz, DMSO) δ 12.52 (s, 1H), 9.15 (s, 1H), 8.90 (t, J = 5.8 Hz, 1H), 8.36 (d, J = 6.5 Hz, 1H), 8.00-7.92 (m, 2H), 7.88 (d, J = 6.7 Hz, 1H), 7.78 (d, J = 5.5 Hz, 2H), 7.46 (dt, J = 13.8, 6.9 Hz, 3H), 6.85 (s, 1H), 6.70 (s, 1H), 5.76 (s, 1H), 4.77 (d, |
| | | | | J = 6.1 Hz, 2H), 4.70 (s, |
| | | | | 2H), 4.56 (d, J = 5.7 Hz, |
| | | | | 2H), 3.62 (td, J = 6.7, |
| | | | | 3.0 Hz, 1H), 3.14 (dt, J = |
| | | | | 10.4, 5.3 Hz, 1H), |
| | | | | 2.96 (dd, J = 14.6, |
| | | | | 10.1 Hz, 2H), 1.69-1.32 (m, |
| | | | | 6H). LC/MS (ESI) |
| | | | | m/z: 555 (M + H)⁺. |
| | | | | RT (Method A): 1.49 min. |
| 478^{e} | 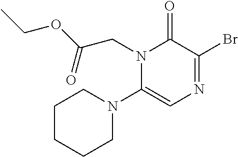 [SMILES: CCOC(=O)Cn1c(N2CCCCC2)cnc(Br)c1=O] | 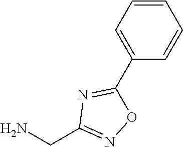 [SMILES: NCc1noc(-c2ccccc2)n1] | 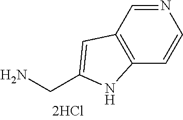 [SMILES: NCc1cc2cnccc2[nH]1] | ¹H NMR (400 MHz, DMSO) δ 12.40 (s, 1H), 9.11 (s, 1H), 8.96-8.85 (m, 1H), 8.33 (d, J = 6.5 Hz, 1H), 8.13- 8.03 (m, 2H), 7.83 (d, J = 6.5 Hz, 1H), 7.71 (t, J = 7.4 Hz, 1H), 7.63 (t, J = 7.6 Hz, 2H), 7.52 (t, J = 6.1 Hz, 1H), 6.82 (s, 1H), 6.63 (s, 1H), 4.71 (s, 2H), 4.66 (d, J = 6.1 Hz, 2H), 4.56 (d, J = 5.7 Hz, 2H), 3.62 |
| | | | | (d, J = 8.5 Hz, 1H), 3.14 (d, |
| | | | | J = 7.4 Hz, 1H), 2.93 (d, J = |
| | | | | 10.8 Hz, 3H), 1.66-1.30 (m, |
| | | | | 6H). LC/MS (ESI) m/z: |
| | | | | 540 (M + H)⁺. |
| | | | | RT (Method A): 1.36 min. |
| 479^{e} | 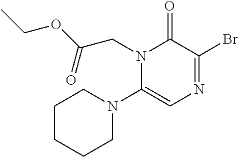 [SMILES: CCOC(=O)Cn1c(N2CCCCC2)cnc(Br)c1=O] | 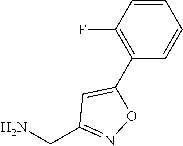 [SMILES: NCc1cc(-c2ccccc2F)on1] | 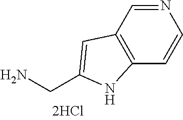 [SMILES: NCc1cc2cnccc2[nH]1] | ¹H NMR (400 MHz, MeOD) δ 8.90 (s, 1H), 8.21 (d, J = 6.7 Hz, 1H), 7.91 (t, J = 7.7 Hz, 1H), 7.77 (d, J = 6.6 Hz, 1H), 7.49 (d, J = 6.4 Hz, 1H), 7.28 (dt, J = 30.3, 8.7 Hz, 2H), 6.86 (s, 1H), 6.75 (d, J = 15.7 Hz, 2H), 4.87 (s, 2H), 4.70 (d, J = 17.1 Hz, |
| | | | | 4H), 3.05 (d, J = 11.3 Hz, |
| | | | | 2H), 2.62 (t, J = 11.6 Hz, 2H), |
| | | | | 1.84-1.38 (m, 6H). LC/MS |
| | | | | (ESI) m/z: 557 (M + H)⁺. |
| | | | | RT (Method A): 1.78 min. |
| 480^{e} | 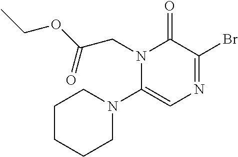 [SMILES: CCOC(=O)Cn1c(N2CCCCC2)cnc(Br)c1=O] | 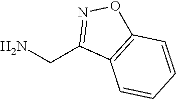 [SMILES: NCc1noc2ccccc12] | 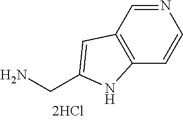 [SMILES: NCc1cc2cnccc2[nH]1] | ¹H NMR (400 MHz, MeOD) δ 8.75 (s, 1H), 8.13 (d, J = 6.0 Hz, 1H), 7.87 (d, J = 8.0 Hz, 1H), 7.59 (d, J = 3.9 Hz, 2H), 7.48 (d, J = 6.0 Hz, 1H), 7.31 (ddd, J = 8.0, 4.6, 3.2 Hz, 1H), 6.72 (s, 1H), 6.63 (s, 1H), 5.00 (s, 3H), 4.84 (s, 2H), 4.62 (s, 2H), 3.02 (d, J = 11.4 Hz, |
| | | | | 2H), 2.60 (t, J = 11.5 Hz, 2H), |
| | | | | 1.60 (td, J = 44.3, 12.9 Hz, |
| | | | | 6H). LC/MS (ESI) |
| | | | | m/z: 513 (M + H)⁺. |
| | | | | RT (Method A): 1.28 min. |
| 484^{e} | 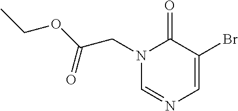 | 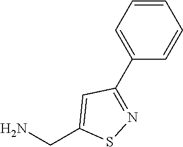 [SMILES: NCc1cc(-c2ccccc2)ns1] | 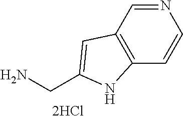 [SMILES: NCc1cc2cnccc2[nH]1] | ¹H NMR (400 MHz, DMSO) δ 11.67 (s, 1H), 9.10 (t, J = 5.7 Hz, 1H), 8.73 (s, 1H), 8.10 (t, J = 5.5 Hz, 1H), 7.97 (d, J = 7.5 Hz, 2H), 7.88 (s, 1H), 7.79 (s, 1H), 7.46 (dt, J = 13.9, 7.2 Hz, 3H), 7.32 (d, J = 5.7 Hz, 1H), 7.10 (s, 1H), 6.47-6.31 |
| | | | | (m, 2H), 4.75-4.59 (m, |
| | | | | 4H), 4.48 (d, J = 5.6 Hz, |
| | | | | 2H). LC/MS (ESI) m/z: |
| | | | | 472 (M + H)⁺. |
| | | | | RT (Method A): 1.11 min. |
| 485^{e} | 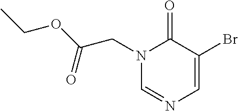 | 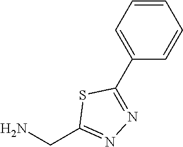 [SMILES: NCc1nnc(-c2ccccc2)s1] | 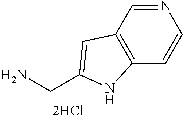 [SMILES: NCc1cc2cnccc2[nH]1] | ¹H NMR (400 MHz, MeOD) δ 8.70 (s, 1H), 8.10 (d, J = 5.8 Hz, 1H), 7.96-7.86 (m, 2H), 7.81 (s, 1H), 7.51 (q, J = 7.1 Hz, 3H), 7.38 (d, J = 5.9 Hz, 1H), 7.22 (s, 1H), 6.55 (s, 1H), 4.85 (s, 2H), 4.72 (s, 2H), 4.62 (s, 2H). |
| | | | | LC/MS (ESI) m/z: 473 |
| | | | | (M + H)⁺. RT (Method A): |
| | | | | 0.72 min. |
| 486^{e} | 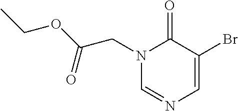 | 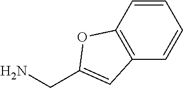 [SMILES: NCc1cc2ccccc2o1] | 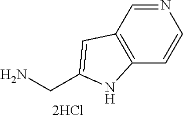 [SMILES: NCc1cc2cnccc2[nH]1] | ¹H NMR (400 MHz, DMSO) δ 11.37 (s, 1H), 8.94- 8.66 (m, 2H), 8.12 (d, J = 5.6 Hz, 1H), 7.76 (s, 1H), 7.55 (dd, J = 21.2, 7.7 Hz, 2H), 7.40-7.07 (m, 4H), 6.80 (s, 1H), 6.44 (s, 1H), 6.09 (t, J = 6.4 Hz, |
| | | | | 1H), 4.64 (s, 2H), 4.54- |
| | | | | 4.35 (m, 4H). LC/MS (ESI) |
| | | | | m/z: 429 (M + H)⁺. |
| | | | | RT (Method A): 1.00 min. |

^{a}Step 1 was performed with Pd₂(dba)₃ and XantPhos in 1,4-dioxane in the presence of CS₂CO₃, Step 2 was performed with TFA in THF/MeOH/H₂O or DCM (for Compounds 236, 244, and 245), and HATU was used in place of TBTU in Step 3.
^{b}Step 1 was performed with Pd₂(dba)₃ and XantPhos in DMF in the presence of CS₂CO₃, Step 2 was performed with TFA in DCM, and HATU was used in place of TBTU in Step 3.
^{c}Step 1 was performed with Pd₂(dba)₃ and XantPhos in 1,4-dioxane in the presence of Cs₂CO₃, Step 2 was performed with NaOH in THF/MeOH/H₂O, and HATU was used in place of TBTU in Step 3.
^{d}Step 2 was performed with LiOH•H₂O in MeOH/H₂O.
^{e}Step 2 was performed with LiOH in THF•MeOH, and HATU was used in place of TBTU in Step 3.

Compound 181 is prepared based on Scheme 14:

| # | Reactant A | Reactant B | Reactant C |
|---|---|---|---|
| 181 | 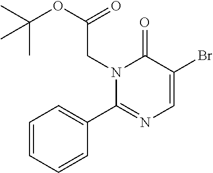 [SMILES: CC(C)(C)OC(=O)Cn1c(-c2ccccc2)ncc(Br)c1=O] | 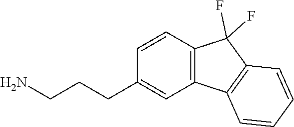 [SMILES: NCCCc1ccc2c(c1)-c1ccccc1C2(F)F] | 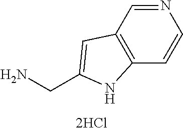 [SMILES: NCc1cc2cnccc2[nH]1] |

### Scheme 15. Synthesis of N-((1H-pyrrolo[3,2-c]pyridin-2-yl)methyl)-2-(5-((3-(4-fluorophenyl)propyl)amino)-6-oxo-2-phenylpyrimidin-1(6H)-yl)acetamide (Compound 46)

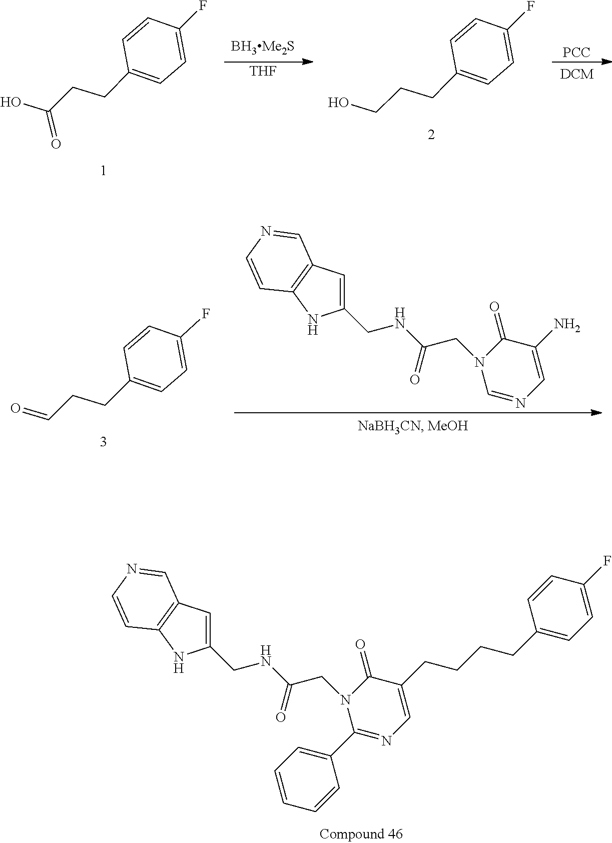

### Step 1: 3-(4-Fluorophenyl)propan-1-ol (2)

To a solution of 3-(4-fluorophenyl)propanoic acid (900 mg, 5.35 mmol) in THF (10 mL) was added BH₃·Me₂S (1 mL, 10 mol/L) drop-wisely at room temperature, the mixture was stirred at 50° C. for 1 hour. The mixture was quenched by drop-wise addition of MeOH at 0° C. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give 3-(4-fluorophenyl)propan-1-ol (600 mg, yield 72.8%) as a colorless oil. ¹H NMR (400 MHz, CDCl₃) δ 7.07 (dd, J=7.9, 5.8 Hz, 2H), 6.89 (t, J=8.6 Hz, 2H), 3.58 (t, J=6.4 Hz, 2H), 2.60 (t, J=7.7 Hz, 2H), 1.83-1.75 (m, 2H).

### Step 2: 3-(4-Fluorophenyl)propanal (3)

To a solution of 3-(4-fluorophenyl)propan-1-ol (425 mg, 2.76 mmol) in DCM (4 mL) was added PCC (1.1 g, 5.19 mmol) under N₂ atmosphere and the reaction mixture was stirred at room temperature for 2 hours. The mixture was concentrated under reduced pressure to dryness. The residue was purified by flash chromatography (silica gel, 0-30% EtOAc in PE) to give 3-(4-fluorophenyl)propanal (250 mg, yield 59.5%) as a colorless oil. ¹H NMR (400 MHz, CDCl₃) δ 9.74 (s, 1H), 7.07 (dd, J=7.8, 5.5 Hz, 2H), 6.91 (d, J=8.6 Hz, 2H), 2.86 (t, J=7.4 Hz, 2H), 2.69 (t, J=7.4 Hz, 2H).

### Step 3: N-((1H-pyrrolo[3,2-c]pyridin-2-yl)methyl)-2-(5-((3-(4-fluorophenyl)propyl) amino)-6-oxo-2-phenylpyrimidin-1(6H)-yl)acetamide (Compound 46)

To a mixture of 3-(4-fluorophenyl)propanal (146 mg, 0.96 mmol) and N-((1H-pyrrolo[3,2-c]pyridin-2-yl)methyl)-2-(5-amino-6-oxo-2-phenylpyrimidin-1(6H)-yl) acetamide (90 mg, 0.24 mmol) in MeOH (3 mL) was added NaBH₃CN (60 mg, 0.96 mmol) under N₂ atmosphere at 0° C. and the reaction mixture was stirred at 50° C. for 2 hours. The mixture was diluted with EtOAc, washed with water and brine, dried over anhydrous Na₂SO₄, filtered, and concentrated under reduced pressure to dryness. The residue was purified by prep-HPLC to give Compound 46 (6.2 mg, yield 5.1%) as a white solid. ¹H NMR (400 MHz, CD₃OD) δ 8.70 (s, 1H), 8.10 (d, J=5.6 Hz, 1H), 7.46 (t, J=7.0 Hz, 3H), 7.40 (d, J=6.1 Hz, 3H), 7.23 (t, J=6.2 Hz, 2H), 7.08 (s, 1H), 6.99 (t, J=8.1 Hz, 2H), 6.47 (s, 1H), 4.62 (s, 2H), 4.54 (s, 2H), 3.18 (t, J=6.9 Hz, 2H), 2.74 (t, J=7.3 Hz, 2H), 2.03-1.94 (m, 2H). LC/MS (ESI) m/z: 511 (M+H)⁺. RT (Method A): 1.56 min.

Compound 88 was prepared based on Scheme 15:

| # | Reactant A | Reactant B | Characterization Data |
|---|---|---|---|
| 88^{a} | 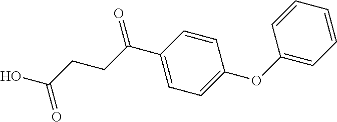 [SMILES: O=C(O)CCC(=O)c1ccc(Oc2ccccc2)cc1] | 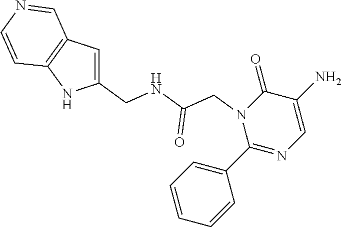 [SMILES: Nc1cnc(-c2ccccc2)n(CC(=O)NCc2cc3cnccc3[nH]2)c1=O] | ¹H NMR (400 MHz, CD₃OD) δ 8.78 (s, 1H), 8.14 (d, J = 6.0 Hz, 1H), 8.01 (d, J = 8.8 Hz, 2H), 7.48 (dd, J = 14.9, 6.5 Hz, 4H), 7.41 (d, J = 8.3 Hz, 4H), 7.23-7.19 (m, 2H), 7.07 (d, J = 7.7 Hz, 2H), 7.00 (d, J = 4.6 Hz, 2H), 6.57 (s, 1H), 4.62 (s, 2H), 4.56 (s, 2H), 3.27 (d, J = 6.9 Hz, 2H), 3.14 (d, J = 6.7 Hz, 2H), 2.13-2.05 (m, 2H). LC/MS (ESI) m/z: 613 (M + H)⁺. RT (Method A): 1.71 min. |

### Scheme 16. Synthesis of N-((1H-pyrrolo[3,2-c]pyridin-2-yl)methyl)-2-(5-((3-(4-cyanophenyl)propyl)amino)-6-oxo-2-phenyl)pyrimidin-1(6H)-yl)acetamide (Compound 48)

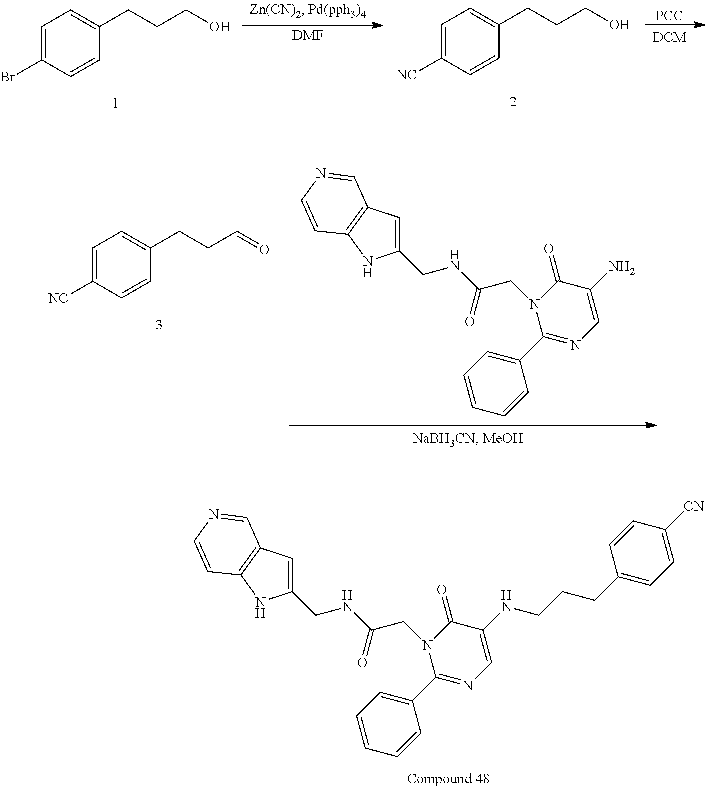

### Step 1: 4-(3-hydroxypropyl)benzonitrile

To a mixture of 3-(4-bromophenyl)propan-1-ol (2.0 g, 9.35 mmol) and Zn(CN)₂ (2.2 g, 18.70 mmol) in DMF (20 mL) was added Pd(PPh₃)₄ (1.1 g, 0.94 mmol) at 0° C. under N₂ atmosphere. The mixture was degassed under N₂ atmosphere for three times and stirred at 110° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-25% EtOAc in PE) to give 4-(3-hydroxypropyl)benzonitrile (500 mg, yield 33.3%) as a colorless oil. LC/MS (ESI) m/z: 162 (M+H)⁺.

### Step 2: 4-(3-oxopropyl)benzonitrile

To a solution of 4-(3-hydroxypropyl)benzonitrile (300 mg, 1.86 mmol) in DCM (5 mL) was added PCC (483 mg, 2.23 mmol) at 0° C. and the mixture was stirred at 0° C. for 2 hours. To the mixture was added silica gel and stirred at 0° C. for 0.5 hour, filtered, and concentrated under reduced pressure to dryness to give 4-(3-oxopropyl)benzonitrile (125 mg, yield 42.2%) as a colorless oil, which was used directly in the next step without further purification.

### Step 3: N-((1H-pyrrolo[3,2-c]pyridin-2-yl)methyl)-2-(5-((3-(4-cyanophenyl)propyl) amino)-6-oxo-2-phenylpyrimidin-1(6H)-yl)acetamide (Compound 48)

To a mixture of N-((1H-pyrrolo[3,2-c]pyridin-2-yl)methyl)-2-(5-amino-6-oxo-2-phenylpyrimidin-1(6H)-yl)acetamide (30 mg, 0.08 mmol) and 4-(3-oxopropyl) benzonitrile (64 mg, 0.40 mmol) in MeOH (3 mL) was added NaBH₃CN (40 mg, 0.64 mmol) under N₂ atmosphere and the reaction mixture was stirred at 50° C. for 1 hour. The mixture was diluted with EtOAc, washed with brine, dried over anhydrous Na₂SO₄, filtered, and concentrated under reduced pressure to dryness. The residue was purified by prep-TLC (DCM:MeOH=10:1) and further purified by prep-HPLC to give Compound 48 (2 mg, yield 4.8%) as a white solid. ¹H NMR (400 MHz, CD₃OD) δ 8.87 (s, 1H), 8.47 (s, 1H), 8.20 (d, J=6.2 Hz, 1H), 7.65 (d, J=8.2 Hz, 3H), 7.50-7.40 (m, 7H), 7.11 (s, 1H), 6.68 (s, 1H), 4.64 (s, 2H), 4.59 (s, 2H), 3.21 (t, J=6.8 Hz, 2H), 2.85 (t, J=7.6 Hz, 2H), 2.08-1.85 (m, 2H). LC/MS (ESI) m/z: 518 (M+H)⁺. RT (Method A): 1.29 min.

### Scheme 17. Synthesis of N-[6-oxo-1-[2-oxo-2-(1H-pyrrolo[3,2-c]pyridin-2-ylmethylamino)ethyl]-2-phenyl-pyrimidin-5-yl]-4-phenoxy-cyclohexanecarboxamide (Compound 49)

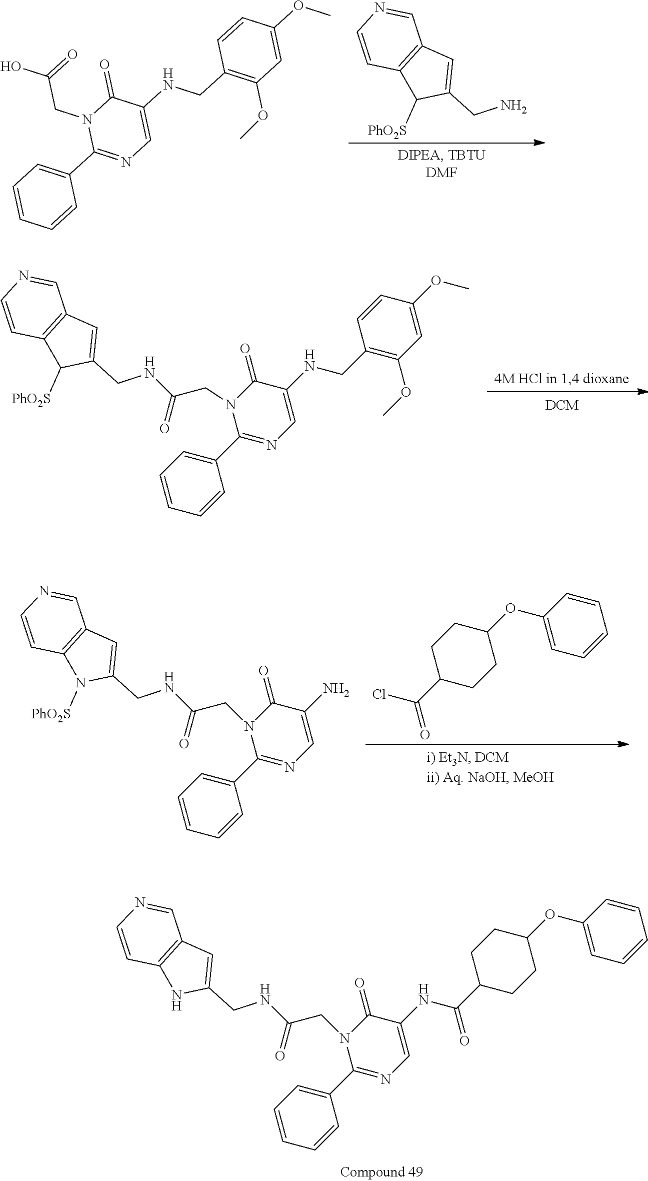

### Step 1: N-[[1-(benzenesulfonyl)pyrrolo[3,2-c]pyridin-2-yl]methyl]-2-[5-[(2,4-dimethoxyphenyl) methylamino]-6-oxo-2-phenyl-pyrimidin-1-yl]acetamide

2-[5-[(2,4-dimethoxyphenyl)methylamino]-6-oxo-2-phenyl-pyrimidin-1-yl]acetic acid (1 g, 2.529 mmol), [1-(benzenesulfonyl)pyrrolo[3,2-c]pyridin-2-yl]methanamine (0.7266 g, 2.529 mmol), TBTU (1.657 g, 5.058 mmol), and DIPEA (0.6537 g, 5.058 mmol) were stirred in DMF (20 mL) at rt for 30 minute. Water (30 mL) was added into the reaction mixture and the solid was filtered. The solid was dissolved in DCM and dried over anhydrous Na₂SO₄, filtered, and concentrated to dryness. The residue was purified by flash chromatography (silica gel, 0-5% MeOH in DCM) to give N-[[1-(benzenesulfonyl) pyrrolo[3,2-c]pyridin-2-yl]methyl]-2-[5-[(2,4-dimethoxyphenyl)methylamino]-6-oxo-2-phenyl-pyrimidin-1-yl]acetamide (1.2 g, 71% yield) as a white solid. LC/MS (ESI) m/z: 665 (M+H)⁺.

### Step 2: 2-(5-amino-6-oxo-2-phenyl-pyrimidin-1-yl)-N-[[1-(benzenesulfonyl)pyrrolo[3,2-c]pyridin-2-yl]methyl]acetamide

N-[[1-(benzenesulfonyl)pyrrolo[3,2-c]pyridin-2-yl]methyl]-2-[5-[(2,4-dimethoxyphenyl)methylamino]-6-oxo-2-phenyl-pyrimidin-1-yl]acetamide (0.15 g, 0.2257 mmol) was stirred in TFA at 60° C. for 1 hr. Excess reagent was evaporated to complete dryness and crude material was washed with saturated NaHCO₃ and extracted with DCM. The organic layer was dried over sodium sulfate and evaporated to dryness to give 2-(5-amino-6-oxo-2-phenyl-pyrimidin-1-yl)-N-[[1-(benzenesulfonyl)pyrrolo[3,2-c]pyridin-2-yl]methyl]acetamide (0.1 g, 90% yield) as a buff white solid. LC/MS (ESI) m/z: 515 (M+H)⁺.

### Step 3: N-[6-oxo-1-[2-oxo-2-(1H-pyrrolo[3,2-c]pyridin-2-ylmethylamino)ethyl]-2-phenyl-pyrimidin-5-yl]-4-phenoxy-cyclohexanecarboxamide (Compound 49)

2-(5-amino-6-oxo-2-phenyl-pyrimidin-1-yl)-N-[[1-(benzenesulfonyl)pyrrolo[3,2-c]pyridin-2-yl]methyl]acetamide (0.01 g, 0.01943 mmol) and triethylamine (0.005 mL, 0.03887 mmol) were stirred in DCM (1 mL) at 0° C. for 5 minute. A solution of 4-phenoxycyclohexanecarbonyl chloride (0.009277 g, 0.03887 mmol) in DCM was added drop wise into the reaction mixture and allowed it to stir for 10 minutes at rt. The solvent was evaporated, and the crude mass was dissolved in MeOH (1 mL). Aq. NaOH (5% in water, 0.5 mL) was added in it and allowed it to stir at 50° C. for 30 minutes. The solvent was evaporated and extracted with DCM. The organic layer was dried over anhydrous Na₂SO₄, filtered, and concentrated to dryness. The residue was purified by flash chromatography (silica gel, 0-3% MeOH in DCM) to give Compound 49 (2 mg, 17.85)% yield). ¹H NMR (400 MHz, MeOD-CDCl₃) δ 8.83 (d, J=5.0 Hz, 1H), 8.62 (s, 1H), 8.43 (s, 3H), 8.02 (s, 1H), 7.41 (t, J=8.2 Hz, 3H), 7.36-7.28 (m, 3H), 7.21-7.10 (m, 2H), 6.83 (td, J=13.1, 7.7 Hz, 3H), 6.39 (s, 1H), 4.57 (s, 2H), 4.52 (s, 1H), 4.45 (s, 2H), 2.56 (s, 1H), 2.13 (s, 1H), 2.03-1.87 (m, 3H), 1.64 (s, 3H), 1.41 (d, J=11.8 Hz, 1H). LC/MS (ESI) m/z: 577 (M+H)⁺. RT (Method A): 1.87 min.

The following compounds were prepared based on Step 3 in Scheme 17:

| # | Reactant A | Reactant B | Characterization Data |
|---|---|---|---|
| 50 | 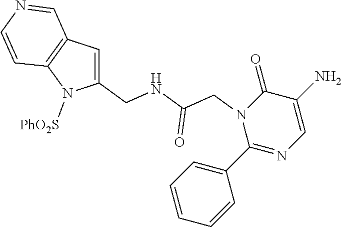 | 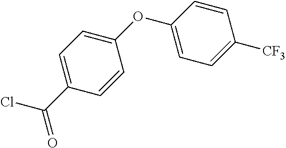 [SMILES: O=C(Cl)c1ccc(Oc2ccc(C(F)(F)F)cc2)cc1] | ¹H NMR (400 MHz, MeOD-CDCl₃) δ 9.08 (s, 1H), 8.87 (s, 1H), 8.20 (d, J = 6.3 Hz, 1H), 8.03 (d, J = 8.7 Hz, 2H), 7.71 (d, J = 8.4 Hz, 2H), 7.63 (d, J = 6.2 Hz, 1H), 7.60-7.50 (m, 3H), 7.46 (t, J = 7.5 Hz, 2H), 7.27-7.15 (m, 4H), 6.69 (s, 1H), 4.73 (s, 2H), 4.61 (s, 2H). LC/MS (ESI) m/z: 639 (M + H)⁺. RT (Method A): 1.97 min. |
| 51 | 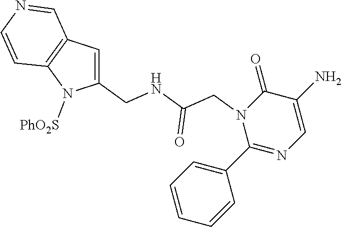 | 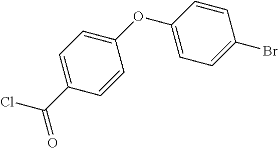 [SMILES: O=C(Cl)c1ccc(Oc2ccc(Br)cc2)cc1] | ¹H NMR (400 MHz, MeOD-CDCl₃) δ 9.07 (s, 1H), 8.86 (s, 1H), 8.19 (d, J = 6.3 Hz, 1H), 7.98 (d, J = 8.7 Hz, 2H), 7.77 (s, 4H), 7.64-7.57 (m, 2H), 7.57-7.50 (m, 4H), 7.46 (t, J = 7.5 Hz, 2H), 7.11 (d, J = 8.7 Hz, 2H), 7.02 (d, J = 8.9 Hz, 2H), 6.68 (s, 1H), 4.61 (s, 2H). LC/MS (ESI) m/z: 650 (M + H)⁺. RT (Method A): 1.89 min. |
| 52 | 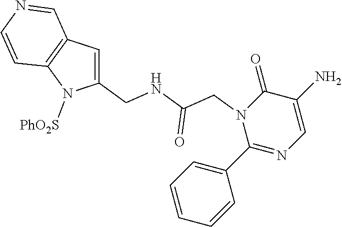 | 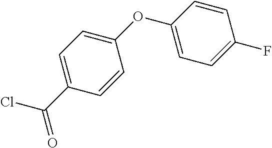 [SMILES: O=C(Cl)c1ccc(Oc2ccc(F)cc2)cc1] | ¹H NMR (400 MHz, MeOD-CDCl₃) δ 9.04 (s, 1H), 8.99 (s, 1H), 8.27 (d, J = 6.5 Hz, 1H), 8.00 (d, J = 8.8 Hz, 2H), 7.78 (d, J = 6.5 Hz, 1H), 7.63-7.45 (m, 6H), 7.24-7.13 (m, 5H), 7.10 (d, J = 8.7 Hz, 2H), 6.81 (s, 1H), 4.76 (s, 3H), 4.64 (s, 3H). LC/MS (ESI) m/z: 589 (M + H)⁺. RT (Method A): 1.75 min. |
| 53 | 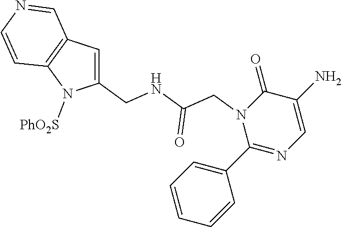 | 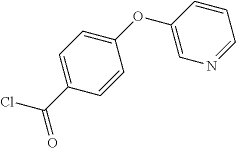 [SMILES: O=C(Cl)c1ccc(Oc2cccnc2)cc1] | ¹H NMR (400 MHz, MeOD-CDCl₃) δ 9.07 (s, 1H), 8.91 (s, 1H), 8.41 (t, J = 3.9 Hz, 3H), 8.22 (d, J = 6.4 Hz, 1H), 8.07-7.99 (m, 2H), 7.70 (d, J = 6.4 Hz, 1H), 7.61-7.43 (m, 7H), 7.21- 7.14 (m, 2H), 6.75 (s, 1H), 4.73 (s, 2H), 4.62 (s, 2H). LC/MS (ESI) m/z: 572 (M + H)⁺. RT (Method A): 1.14 min. |

### Scheme 18. Synthesis of N-(1-(2-(((1H-pyrrolo[3,2-c]pyridin-2-yl)methyl)amino)-2-oxoethyl)-6-oxo-2-phenyl-1,6-dihydropyrimidin-5-yl)-4-(2,4-difluorophenoxy)benzamide (Compound 55)

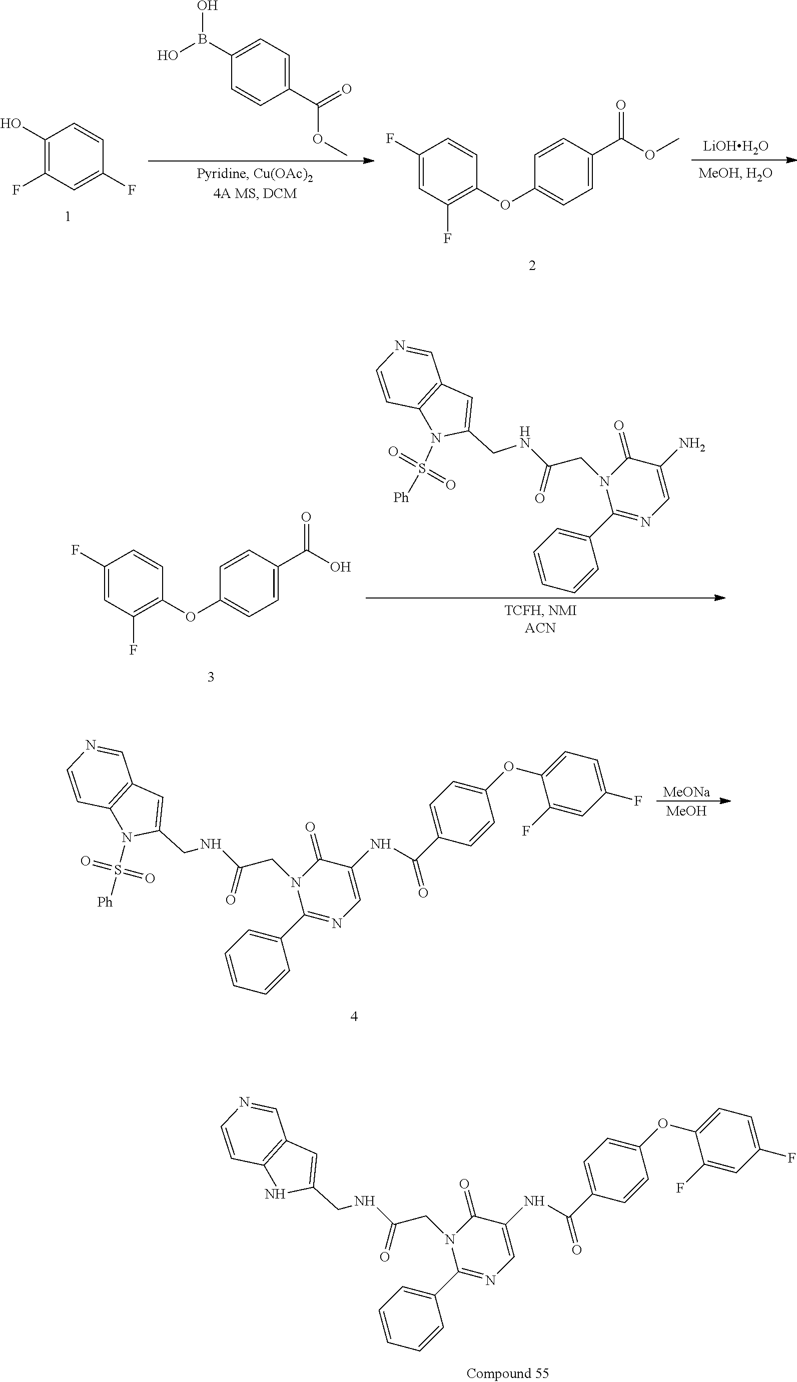

### Step 1: Methyl 4-(2,4-difluorophenoxy)benzoate (2)

To a mixture of 2,4-difluorophenol (500 mg, 3.85 mmol) and (4-(methoxycarbonyl) phenyl)boronic acid (1.04 g, 5.78 mmol) in DCM (15 mL) was added pyridine (0.84 mL, 7.70 mmol), Cu(OAc)₂ (1.05 g, 5.78 mmol) and 4 A molecular sieves (3.0 g) at 0° C. and the mixture was stirred under O₂ atmosphere at room temperature overnight. The mixture was filtered, and the filtrate was concentrated to dryness. The residue was purified by flash chromatography (silica gel, 0-4% EtOAc in PE) to give the title compound (790 mg, yield 77.8%) as a light-yellow oil. ¹H NMR (400 MHz, CDCl₃) δ 7.98 (d, J=8.9 Hz, 2H), 7.15-7.08 (m, 1H), 6.99-6.83 (m, 4H), 3.87 (s, 3H). LC/MS (ESI) m/z: 265 (M+H)⁺.

### Step 2: 4-(2,4-Difluorophenoxy)benzoic acid (3)

To a solution of methyl 4-(2,4-difluorophenoxy)benzoate (790 mg, 2.99 mmol) in MeOH (10 mL) and water (4 mL) was added LiOH H₂O (450 g, 10.72 mmol) and the mixture was stirred at room temperature for 4 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (650 mg, yield 86.9%) as a white solid, which was used directly in the next step without further purification. LC/MS (ESI) m/z: 251 (M+H)⁺.

### Step 3: 4-(2,4-Difluorophenoxy)-N-(6-oxo-1-(2-oxo-2-(((1-(phenylsulfonyl)-1H-pyrrolo[3,2-c]pyridin-2-yl)methyl)amino)ethyl)-2-phenyl-1,6-dihydropyrimidin-5-yl)benzamide (4)

To a mixture of 4-(2,4-difluorophenoxy)benzoic acid (200 mg, 0.80 mmol) and 2-(5-amino-6-oxo-2-phenylpyrimidin-1(6H)-yl)-N-((1-(phenylsulfonyl)-1H-pyrrolo[3,2-c]pyridin-2-yl)methyl)acetamide (270 mg, 0.52 mmol) in MeCN (5 mL) was added TCFH (440 mg, 1.57 mmol) and NMI (130 mg, 1.57 mmol) under N₂ atmosphere and the reaction mixture was stirred at room temperature for 1 hour. The mixture was diluted with DCM, washed with 10% aq.NaHCO₃ solution and brine, dried over anhydrous Na₂SO₄, filtered, and concentrated under reduced pressure to dryness. The residue was purified by flash chromatography (silica gel, 0-4% MeOH in DCM) to give the title compound (260 mg, yield 66.3%) as a yellow solid. LC/MS (ESI) m/z: 747 (M+H)⁺.

### Step 4: N-(1-(2-(((1H-pyrrolo[3,2-c]pyridin-2-yl)methyl)amino)-2-oxoethyl)-6-oxo-2-phenyl-1,6-dihydropyrimidin-5-yl)-4-(2,4-difluorophenoxy)benzamide (Compound 55)

A solution of 4-(2,4-difluorophenoxy)-N-(6-oxo-1-(2-oxo-2-(((1-(phenylsulfonyl)-1H-pyrrolo[3,2-c]pyridin-2-yl)methyl)amino)ethyl)-2-phenyl-1,6-dihydropyrimidin-5-yl)benzamide (260 mg, 0.35 mmol) in MeONa/MeOH solution (7.0 mL, 0.5 M) and the mixture was stirred at room temperature for 2 hours. The reaction mixture was diluted with water and extracted with DCM twice. The combined organic layers were washed with brine, dried over anhydrous Na₂SO₄, filtered, and concentrated under reduced pressure to dryness. The residue was purified by prep-HPLC to give Compound 55 (30 mg, yield 14.2%) as a white solid. ¹H NMR (400 MHz, DMSO-d₆) δ 11.34 (s, 1H), 9.48 (s, 1H), 8.83-8.71 (m, 3H), 8.11 (d, J=5.6 Hz, 1H), 8.04-7.97 (m, 2H), 7.59-7.52 (m, 4H), 7.49-7.41 (m, 3H), 7.34-7.30 (m, 1H), 7.24-7.17 (m, 1H), 7.11-7.05 (m, 2H), 6.32 (s, 1H), 4.58 (s, 2H), 4.43 (d, J=5.5 Hz, 2H). LC/MS (ESI) m/z: 607 (M+H)⁺. RT (Method A): 1.73 min.

The following compounds were prepared based on Steps 3 and 4 in Scheme 18:

| # | Reactant A | Reactant B | Characterization Data |
|---|---|---|---|
| 54^{a,b} | 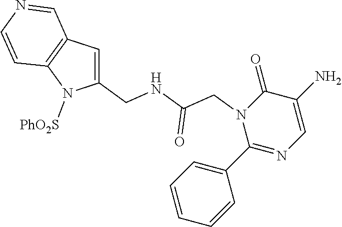 | 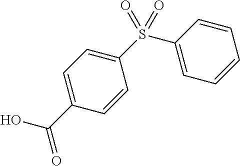 [SMILES: O=C(O)c1ccc(S(=O)(=O)c2ccccc2)cc1] | ¹H NMR (400 MHz, MeOD-CDCl₃) δ 9.06 (s, 1H), 8.12 (s, 4H), 7.99 (dq, J = 9.2, 6.4 Hz, 3H), 7.70-7.51 (m, 7H), 7.46 (dd, J = 8.8, 6.2 Hz, 2H), 6.69 (s, 1H), 4.71 (s, 3H), 4.61 (s, 2H). LC/MS (ESI) m/z: 619 (M + H)⁺. |
| 56^{a,b} | 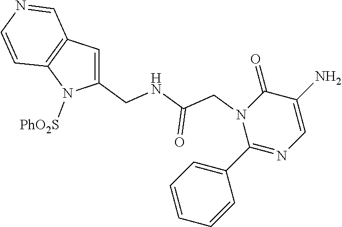 | 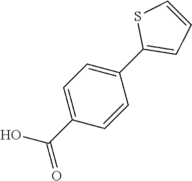 [SMILES: O=C(O)c1ccc(-c2cccs2)cc1] | ¹H NMR (400 MHz, MeOD-CDCl₃) δ 9.14 (s, 1H), 8.89 (s, 1H), 8.21 (d, J = 6.3 Hz, 1H), 7.99 (d, J = 8.1 Hz, 2H), 7.81 (d, J = 8.1 Hz, 2H), 7.70 (s, 1H), 7.62- 7.42 (m, 7H), 7.15 (dd, J = 5.1, 3.6 Hz, 1H), 6.75 (s, 1H), 4.73 (s, 2H), 4.64 (s, 2H). LC/MS (ESI) m/z: 561 (M + H)⁺. RT (Method A): 1.66 min. |
| 57^{a,b} | 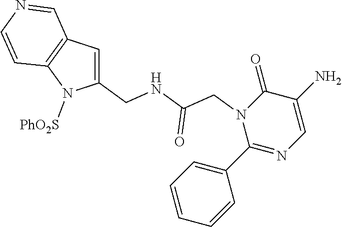 | 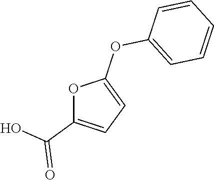 [SMILES: O=C(O)c1ccc(Oc2ccccc2)o1] | ¹H NMR (400 MHz, MeOD-CDCl₃) δ 9.03 (s, 1H), 8.92 (s, 1H), 8.37 (s, 2H), 8.21 (d, J = 6.4 Hz, 1H), 7.76 (s, 1H), 7.61-7.51 (m, 3H), 7.51-7.40 (m, 4H), 7.32 (d, J = 3.7 Hz, 1H), 7.30- 7.15 (m, 3H), 6.79 (s, 1H), 5.71 (d, J = 3.7 Hz, 1H), 4.72 (s, 5H), 4.64 (s, 2H). LC/MS (ESI) m/z: 561 (M + H)⁺. RT (Method A): 1.47 min. |
| 58^{a,b} | 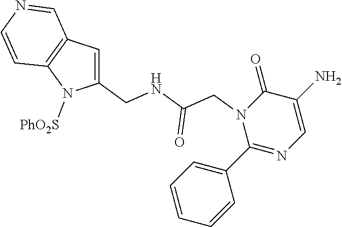 | 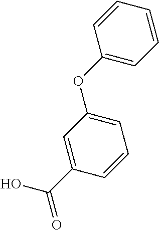 [SMILES: O=C(O)c1cccc(Oc2ccccc2)c1] | ¹H NMR (400 MHz, MeOD-CDCl₃) δ 9.04 (s, 1H), 8.87 (s, 1H), 8.19 (d, J = 6.3 Hz, 1H), 7.67 (dd, J = 16.3, 7.0 Hz, 2H), 7.62- 7.49 (m, 6H), 7.43 (dt, J = 25.2, 7.7 Hz, 5H), 7.28-7.14 (m, 2H), 7.06 (d, J = 8.1 Hz, 2H), 6.70 (s, 1H), 4.72 (s, 2H), 4.61 (s, 2H). LC/MS (ESI) m/z: 571 (M + H)⁺. RT (Method A): 1.69 min. |
| 59^{a,b} | 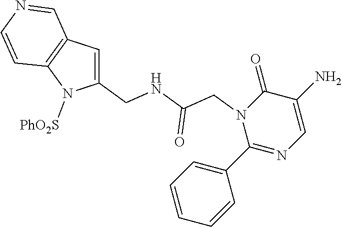 | 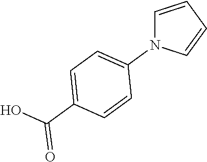 [SMILES: O=C(O)c1ccc(-n2cccc2)cc1] | ¹H NMR (400 MHz, MeOD-CDCl₃) δ 9.07 (s, 1H), 8.91 (s, 1H), 8.43 (s, 2H), 8.22 (s, 1H), 8.07 (d, J = 8.6 Hz, 2H), 7.76-7.62 (m, 3H), 7.56 (dt, J = 9.6, 6.9 Hz, 3H), 7.47 (t, J = 7.5 Hz, 2H), 7.31 (t, J = 2.2 Hz, 2H), 6.75 (s, 1H), 6.36 (t, J = 2.2 Hz, 2H), 4.74 (s, 3H), 4.62 (s, 2H). LC/MS (ESI) m/z: 544 (M + H)⁺. RT (Method A): 1.48 min. |
| 72^{a,b} | 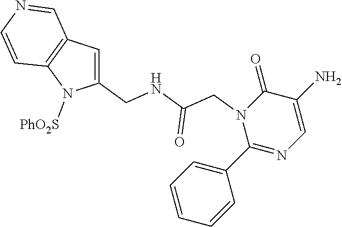 | 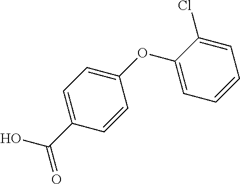 [SMILES: O=C(O)c1ccc(Oc2ccccc2Cl)cc1] | ¹H NMR (400 MHz, MeOD-CDCl₃) δ 9.05 (s, 1H), 8.86 (s, 1H), 8.49 (s, 1H), 8.19 (d, J = 6.2 Hz, 1H), 7.97 (d, J = 8.8 Hz, 2H), 7.63-7.44 (m, 6H), 7.38 (dd, J = 7.9, 1.6 Hz, 1H), 7.24 (ddd, J = 31.2, 7.9, 1.5 Hz, 2H), 7.02 (d, J = 8.7 Hz, 2H), 6.67 (s, 1H), 4.72 (s, 2H). LC/MS (ESI) m/z: 606 (M + H)⁺. RT (Method A): 1.70 min. |
| 73^{a,b} | 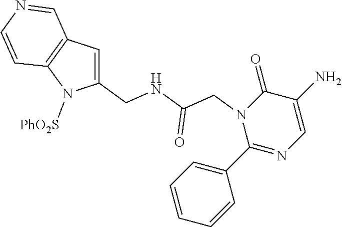 | 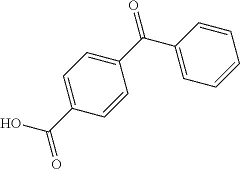 [SMILES: O=C(O)c1ccc(C(=O)c2ccccc2)cc1] | ¹H NMR (400 MHz, MeOD-CDCl₃) δ 9.12 (s, 1H), 8.93 (s, 1H), 8.37 (s, 2H), 8.23 (d, J = 6.4 Hz, 1H), 8.11 (d, J = 8.2 Hz, 2H), 7.93 (d, J = 8.1 Hz, 2H), 7.86- 7.79 (m, 2H), 7.76 (d, J = 6.5 Hz, 1H), 7.69 (t, J = 7.4 Hz, 1H), 7.62-7.52 (m, 5H), 7.48 (t, J = 7.5 Hz, 2H), 6.80 (s, 1H), 4.64 (s, 2H). LC/MS (ESI) m/z: 583 (M + H)⁺. RT (Method A): 1.49 min. |
| 74^{a,b} | 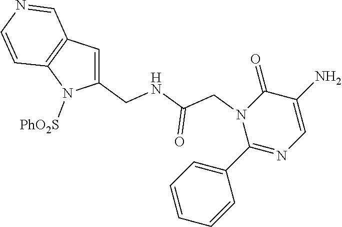 | 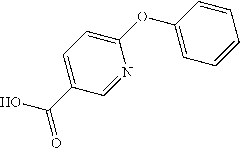 [SMILES: O=C(O)c1ccc(Oc2ccccc2)nc1] | ¹H NMR (400 MHz, MeOD-CDCl₃) δ 9.06 (s, 1H), 8.84 (s, 1H), 8.76 (d, J = 2.5 Hz, 1H), 8.51 (s, 2H), 8.34 (dd, J = 8.7, 2.6 Hz, 1H), 8.18 (s, 1H), 7.67-7.50 (m, 4H), 7.47 (dt, J = 8.0, 4.1 Hz, 4H), 7.29 (t, J = 7.4 Hz, 1H), 7.21- 7.15 (m, 2H), 7.06 (d, J = 8.7 Hz, 1H), 6.67 (s, 1H), 4.61 (s, 2H). LC/MS (ESI) m/z: 572 (M + H)⁺. RT (Method A): 1.29 min. |
| 75^{a,b} | 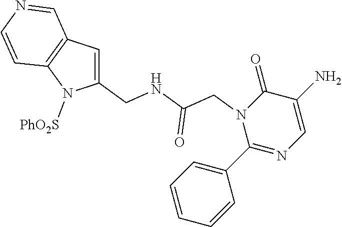 | 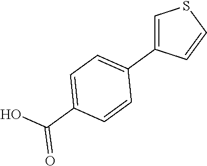 [SMILES: O=C(O)c1ccc(-c2ccsc2)cc1] | ¹H NMR (400 MHz, MeOD-CDCl₃) δ 9.13 (s, 1H), 8.86 (s, 1H), 8.45 (s, 3H), 8.19 (d, J = 6.2 Hz, 1H), 8.01 (d, J = 8.3 Hz, 2H), 7.83 (d, J = 8.4 Hz, 2H), 7.75 (dd, J = 2.8, 1.5 Hz, 1H), 7.63- 7.56 (m, 3H), 7.53 (tt, J = 5.4, 2.7 Hz, 3H), 7.47 (q, J = 6.7 Hz, 2H), 6.69 (s, 1H), 4.62 (s, 2H). LC/MS (ESI) m/z: 561 (M + H)⁺. RT (Method A): 1.55 min. |
| 92^{a,b} | 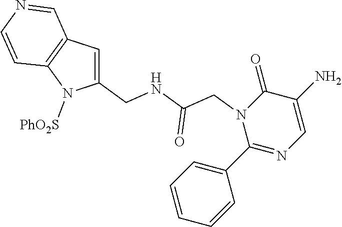 | 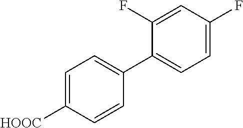 [SMILES: O=C(O)c1ccc(-c2ccc(F)cc2F)cc1] | ¹H NMR (400 MHz, MeOD-CDCl3) δ 9.16 (s, 1H), 8.81 (s, 1H), 8.17 (d, J = 6.1 Hz, 1H), 8.05 (d, J = 8.1 Hz, 2H), 7.71 (d, J = 7.8 Hz, 2H), 7.63-7.51 (m, 6H), 7.48 (d, J = 7.5 Hz, 2H), 7.12- 6.96 (m, 2H), 6.63 (s, 1H), 4.62 (s, 2H). LC/MS (ESI) m/z: 591 (M + H)⁺. RT (Method A): 1.53 min. |
| 93^{b} | 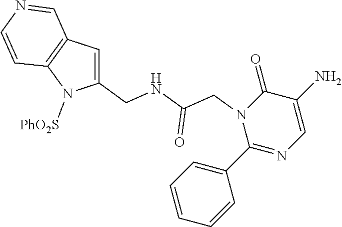 | 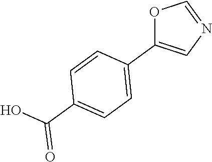 [SMILES: O=C(O)c1ccc(-c2cnco2)cc1] | ¹H NMR (400 MHz, MeOD-CDCl₃) δ 9.19 (s, 1H), 8.73 (s, 1H), 8.20 (s, 1H), 8.14 (d, J = 5.9 Hz, 1H), 8.06 (d, J = 8.3 Hz, 2H), 7.88 (d, J = 8.3 Hz, 2H), 7.60 (s, 1H), 7.58 (s, 2H), 7.52 (d, J = 7.6 Hz, 1H), 7.44 (t, J = 7.6 Hz, 2H), 7.35 (d, J = 5.8 Hz, 1H), 6.49 (s, 1H), 4.70 (s, 3H), 4.59 (s, 2H). LC/MS (ESI) m/z: 546 (M + H)⁺. RT (Method A): 0.97 min. |
| 94^{a,b} | 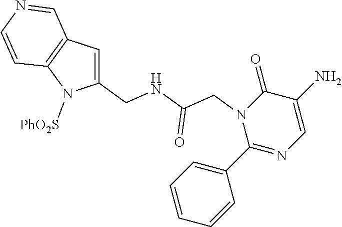 | 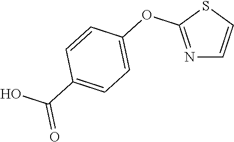 [SMILES: O=C(O)c1ccc(Oc2nccs2)cc1] | ¹H NMR (400 MHz, MeOD-CDCl₃) δ 9.12 (s, 1H), 8.82 (s, 1H), 8.57 (s, 1H), 8.18 (d, J = 6.2 Hz, 1H), 8.10-8.01 (m, 2H), 7.62- 7.50 (m, 4H), 7.46 (dd, J = 8.3, 6.6 Hz, 4H), 7.31 (d, J = 3.8 Hz, 1H), 7.10 (d, J = 3.8 Hz, 1H), 6.65 (s, 1H), 4.72 (s, 2H), 4.61 (s, 2H). LC/MS (ESI) m/z: 578 (M + H)⁺. RT (Method A): 1.14 min. |
| 102^{a,b} | 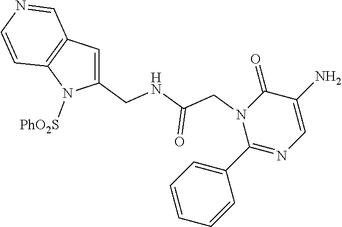 | 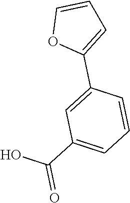 [SMILES: O=C(O)c1cccc(-c2ccco2)c1] | ¹H NMR (400 MHz, MeOD-CDCl₃) δ 9.15 (s, 1H), 8.71 (s, 1H), 8.27 (t, J = 1.9 Hz, 1H), 8.10 (d, J = 5.8 Hz, 1H), 7.97-7.76 (m, 3H), 7.62-7.49 (m, 5H), 7.49- 7.39 (m, 3H), 7.36 (d, J = 5.8 Hz, 1H), 6.99 (d, J = 21.2 Hz, 1H), 6.89 (d, J = 3.4 Hz, 1H), 6.56 (dd, J = 3.4, 1.8 Hz, 1H), 6.49 (s, 1H), 4.71 (s, 2H), 4.59 (s, 2H). LC/MS (ESI) m/z: 545 (M + H)⁺. RT (Method A): 1.37 min. |
| 106 | 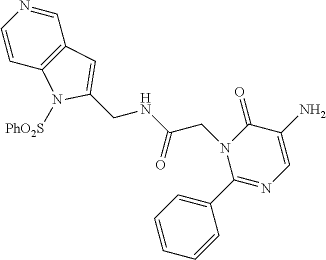 | 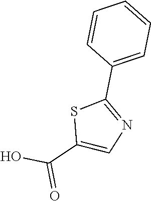 [SMILES: O=C(O)c1cnc(-c2ccccc2)s1] | LC/MS (ESI) m/z: 562 (M + H)⁺. RT (Method A): 1.44 min. |
| 107 | 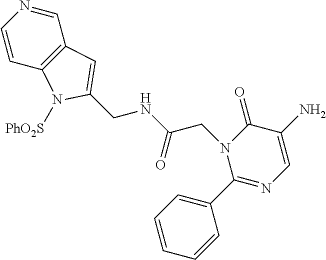 | 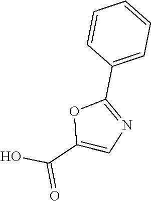 [SMILES: O=C(O)c1cnc(-c2ccccc2)o1] | LC/MS (ESI) m/z: 546 (M + H)⁺. RT (Method A): 1.22 min. |
| 108 | 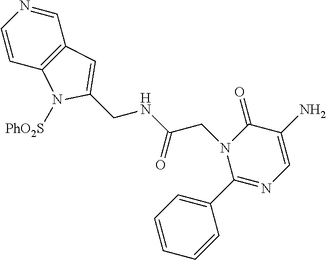 | 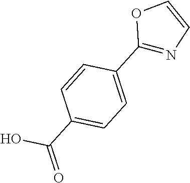 [SMILES: O=C(O)c1ccc(-c2ncco2)cc1] | LC/MS (ESI) m/z: 546 (M + H)⁺. RT (Method A): 0.98 min. |
| 109^{a,b} | 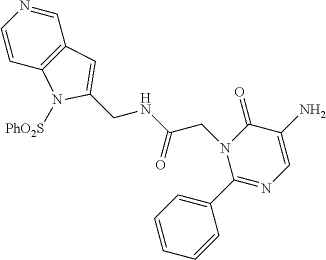 | 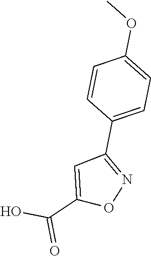 | ¹H NMR (400 MHz, MeOD-CDCl3) δ 9.11 (s, 1H), 8.88 (s, 1H), 8.25 (s, 1H), 7.84 (d, J = 8.8 Hz, 2H), 7.74 (s, 1H), 7.60-7.44 (m, 7H), 7.06 (d, J = 8.8 Hz, 2H), 6.76 (s, 1H), 4.73 (s, 4H), 3.90 (s, 3H). LC/MS (ESI) m/z: 576 (M + H)⁺. RT (Method A): 1.35 min. |
| 110^{a,b} | 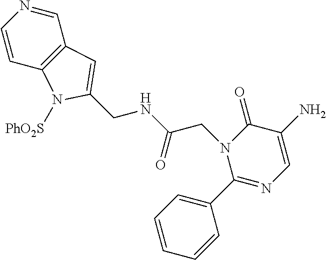 | 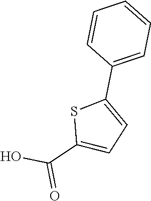 [SMILES: O=C(O)c1ccc(-c2ccccc2)s1] | ¹H NMR (400 MHz, MeOD-CDCl₃) δ 9.10 (s, 1H), 8.79 (s, 1H), 8.47 (s, 1H), 8.16 (d, J = 6.0 Hz, 1H), 7.80 (d, J = 4.0 Hz, 1H), 7.73-7.65 (m, 2H), 7.58 (s, 1H), 7.53-7.37 (m, 9H), 6.59 (s, 1H), 4.70 (s, 2H), 4.61 (s, 2H). LC/MS (ESI) m/z: 561 (M + H)⁺. RT (Method A): 1.52 min. |
| 111^{a,b} | 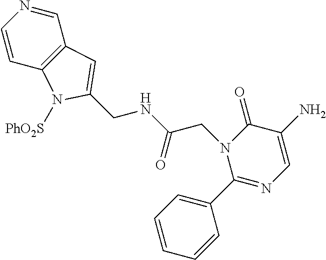 | 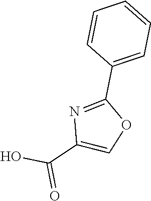 [SMILES: O=C(O)c1coc(-c2ccccc2)n1] | ¹H NMR (400 MHz, MeOD-CDCl₃) δ 9.17 (s, 1H), 8.88 (s, 1H), 8.57 (s, 1H), 8.14 (dd, J = 7.5, 2.0 Hz, 3H), 7.66-7.41 (m, 9H), 6.74 (s, 1H), 4.75 (s, 2H), 4.66 (s, 2H). LC/MS (ESI) m/z: 546 (M + H)⁺. RT (Method A): 1.28 min. |
| 115^{a,b} | 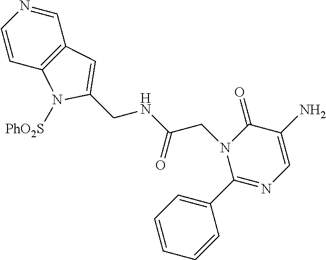 | 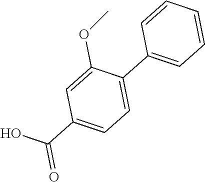 | ¹H NMR (400 MHz, MeOD-CDCl₃) δ 9.18 (s, 1H), 8.86 (s, 1H), 8.39 (s, 1H), 8.20 (d, J = 6.3 Hz, 1H), 7.64 (d, J = 1.7 Hz, 2H), 7.61-7.51 (m, 6H), 7.50-7.35 (m, 6H), 6.73 (s, 1H), 4.74 (s, 3H), 3.93 (s, 3H). LC/MS (ESI) m/z: 585 (M + H)⁺. RT (Method A): 1.65 min. |
| 206 | 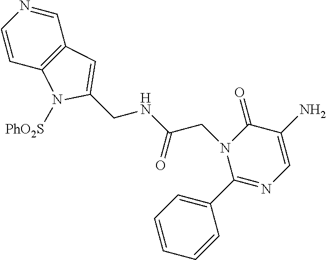 | 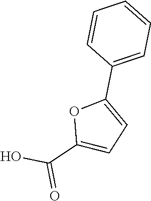 [SMILES: O=C(O)c1ccc(-c2ccccc2)o1] | ¹H NMR (400 MHz, DMSO-d₆) δ 11.35 (s, 1H), 9.40 (s, 1H), 8.80 (d, J = 6.1 Hz, 2H), 8.75 (s, 1H), 8.11 (d, J = 5.7 Hz, 1H), 7.91 (d, J = 7.5 Hz, 2H), 7.58 (d, J = 7.0 Hz, 2H), 7.54 (d, J = 7.7 Hz, 2H), 7.50 (d, J = 8.4 Hz, 2H), 7.49-7.40 (m, 3H), 7.33 (d, J = 5.7 Hz, 1H), 7.23 (d, J = 3.6 Hz, 1H), 6.35 (s, 1H), 4.60 (s, 2H), 4.44 (s, 2H). LC/MS (ESI) m/z: 545 (M + H)⁺. RT (Method A): 1.37 min. |
| 208 | 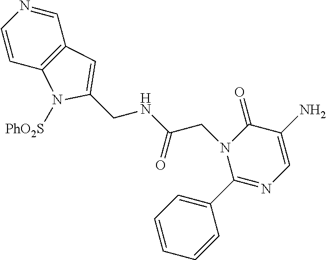 | 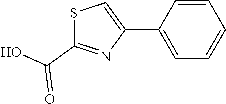 [SMILES: O=C(O)c1nc(-c2ccccc2)cs1] | ¹H NMR (400 MHz, CD₃OD) δ 9.10 (s, 1H), 8.73 (s, 1H), 8.26 (s, 1H), 8.08-8.02 (m, 3H), 7.59 (d, J = 7.0 Hz, 2H), 7.55- 7.41 (m, 7H), 6.53 (s, 1H), 4.75 (s, 2H), 4.60 (s, 2H). LC/MS (ESI) m/z: 562 (M + H)⁺. RT (Method A): 1.38 min. |
| 210 | 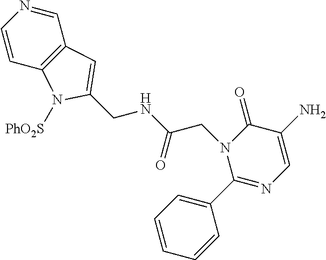 | 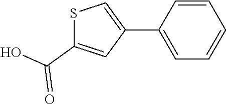 [SMILES: O=C(O)c1cc(-c2ccccc2)cs1] | ¹H NMR (400 MHz, CD₃OD) δ 8.97 (s, 1H), 8.72 (d, J = 0.9 Hz, 1H), 8.31 (M, 1H), 8.09 (d, J = 5.8 Hz, 1H), 8.00 (d, J = 1.4 Hz, 1H), 7.75-7.72 (m, 2H), 7.58- 7.51 (m, 3H), 7.45 (t, J = 7.6 Hz, 4H), 7.39 (d, J = 5.8 Hz, 1H), 7.34 (t, J = 7.4 Hz, 1H), 6.49 (m, 1H), 4.72 (s, 2H), 4.56 (s, 2H). LC/MS (ESI) m/z: 561 (M + H)⁺. RT (Method A): 1.49 min. |
| 227^{c} | 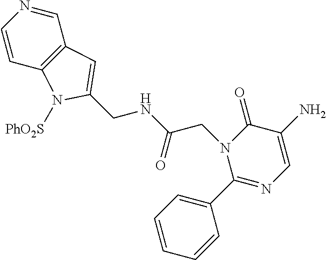 | 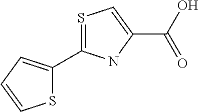 | ¹H-NMR (400 MHz, DMSO-d6) δ 11.34 (s, 1H), 9.77 (s, 1H), 9.00 (s, 1H), 8.83-8.68 (m, 2H), 8.52 (s, 1H), 8.11 (d, J = 5.6 Hz, 1H), 7.84 (td, J = 4.4, 1.2 Hz, 2H), 7.62-7.42 (m, 5H), 7.38-7.18 (m, 2H), 6.36 (s, 1H), 4.61 (s, 2H), 4.45 (d, J = 5.6 Hz, 2H). LC/MS (ESI) m/z = 568 (M + H)⁺. RT (Method A): 1.36 min. |
| 409 | 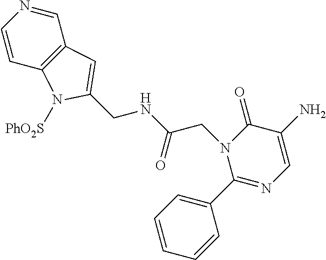 | 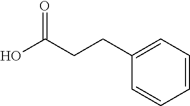 [SMILES: O=C(O)CCc1ccccc1] | ¹H NMR (400 MHz, DMSO-d₆) δ 11.39 (s, 1H), 9.32 (s, 1H), 8.96-8.89 (m, 1H), 8.73 (s, 1H), 8.57 (s, 1H), 8.11 (d, J = 5.6 Hz, 1H), 7.35-7.14 (m, 6H), 6.44 (s, 1H), 4.68 (s, 2H), 4.47 (d, J = 4.0 Hz, 2H), 3.64-3.54 (m, 4H), 3.02-2.91 (m, 4H), 2.89-2.82 (m, 2H), 2.75-2.69 (m, 2H). LC/MS (ESI) m/z: 516 (M + H)⁺. RT (Method A): 0.88 min. |
| 410 | 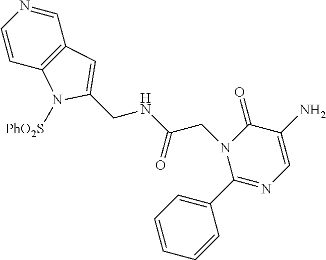 | 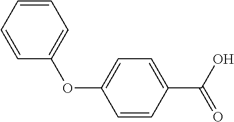 [SMILES: O=C(O)c1ccc(Oc2ccccc2)cc1] | ¹H NMR(400 MHz, DMSO-d₆) δ 11.35 (s, 1H), 9.30 (s, 1H), 8.91 (t, J = 5.1 Hz, 1H), 8.73 (s, 1H), 8.46 (s, 1H), 8.10 (d, J = 5.6 Hz, 1H), 8.00-7.96 (m, 2H), 7.48-7.44 (m, 2H), 7.31 (d, J = 5.3 Hz, 1H), 7.24 (t, J = 7.5 Hz, 1H), 7.14-7.11 (m, 2H), 7.09-7.06 (m, 2H), 6.45 (s, 1H), 4.71 (s, 2H), 4.48 (d, J = 5.3 Hz, 2H), 3.66-3.62 (m, 4H), 3.09- 3.05 (m, 4H). LC/MS (ESI) (m/z): 580 (M + H)⁺. RT (Method A): 1.33 min. |
| 411 | 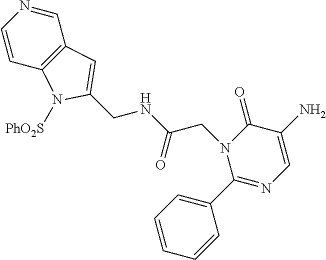 | 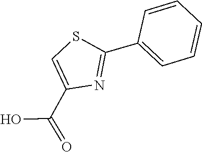 [SMILES: O=C(O)c1csc(-c2ccccc2)n1] | ¹H NMR (400 MHz, CD₃OD) δ 8.76 (s, 1H), 8.70 (s, 1H), 8.23 (d, J = 1.4 Hz, 1H), 8.21 (d, J = 1.9 Hz, 1H), 8.11 (d, J = 5.8 Hz, 1H), 8.05 (s, 1H), 7.65-7.63 (m, 2H), 7.62 (s, 1H), 7.44 (d, J = 5.8 Hz, 1H), 6.63 (s, 1H), 4.87 (s, 2H), 4.65 (s, 2H), 3.76- 3.74 (m, 4H), 3.22-3.19 (m, 4H). LC/MS (ESI) (m/z): 571 (M + H)⁺. RT (Method A): 1.23 min. |
| 413 | 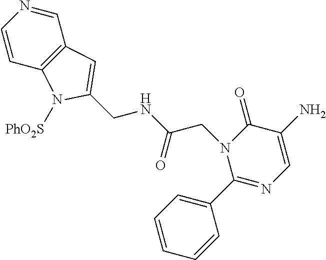 | 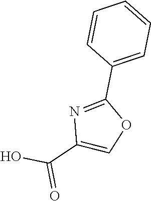 [SMILES: O=C(O)c1coc(-c2ccccc2)n1] | ¹H NMR (400 MHz, DMSO-d₆) δ 11.67 (s, 1H), 9.20 (s, 1H), 8.96 (s, 1H), 8.74 (d, J = 4.2 Hz, 2H), 8.12-7.99 (m, 4H), 7.63- 7.60 (m, 3H), 7.32 (d, J = 5.5 Hz, 1H), 6.47 (s, 1H), 4.74 (s, 2H), 4.50 (d, J = 5.1 Hz, 2H), 3.66-3.61 (m, 4H), 3.08-3.03 (m, 4H). LC/MS (ESI) m/z: 555 (M + H)⁺. RT (Method A): 1.13 min. |

^{a}LiOH was used in place of NaOMe for deprotection.
^{b}The intermediate from the coupling reaction was not isolated prior to deprotection.
^{c}Step 4 was performed with NaOH in DMSO.

Compounds 163 and 173 are prepared based on Steps 3 and 4 in Scheme 18:

| # | Reactant A | Reactant B |
|---|---|---|
| 163 | 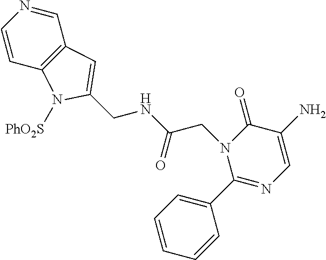 | 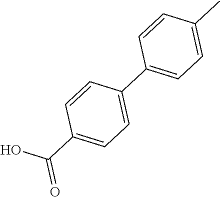 [SMILES: Cc1ccc(-c2ccc(C(=O)O)cc2)cc1] |
| 173 | 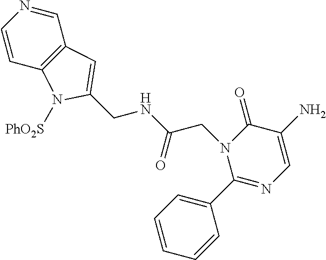 | 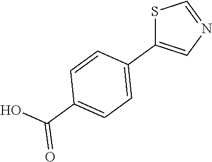 [SMILES: O=C(O)c1ccc(-c2cncs2)cc1] |

### Scheme 19. Synthesis of N-((1H-pyrrolo[3,2-c]pyridin-2-yl)methyl)-2-(3-(([1,1′-biphenyl]-4-ylmethyl)amino)-2-oxo-6-phenylpyrazin-1(2H)-yl)acetamide (Compound 60)

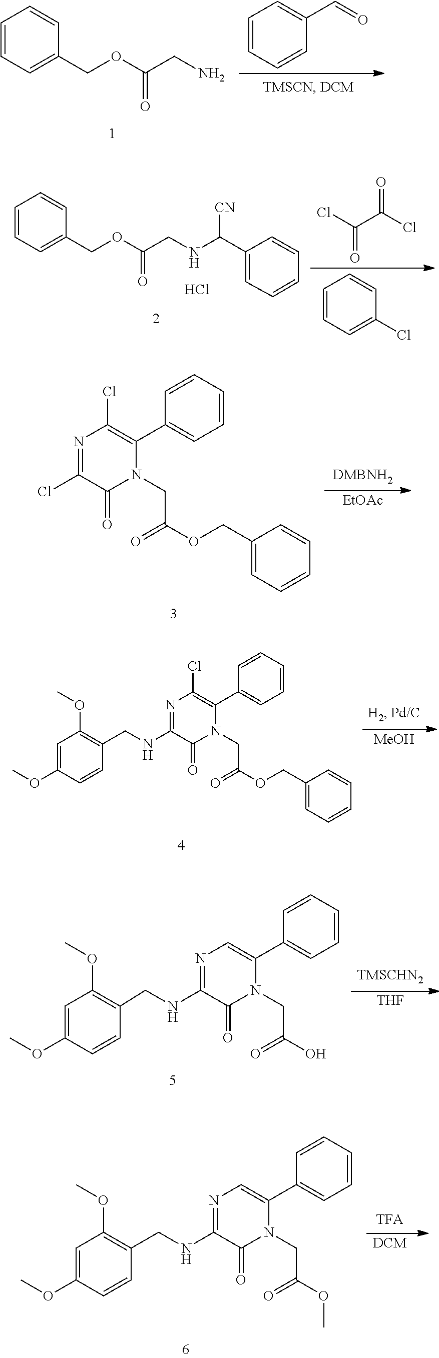

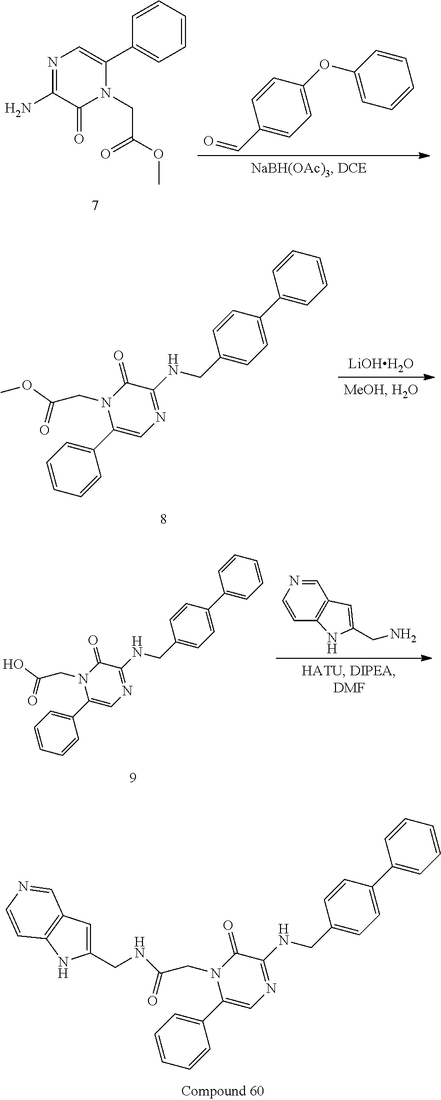

### Step 1: Benzyl (cyano(phenyl)methyl)glycinate hydrochloride (2)

To a solution of benzyl glycinate (8.5 g, 51.5 mmol) in DCM (15 mL) was added benzaldehyde (5.5 g, 51.5 mmol) in portions followed by drop-wise addition of TMSCN (5.61 g, 56.7 mmol) over 10 minutes at room temperature. The reaction mixture was stirred at room temperature for 4 hours. The mixture was concentrated under reduced pressure to dryness and the residue was dissolved in EtOAc. The mixture was washed with water and 10N aq. HCl was added drop-wisely at 0° C. The formed precipitate was collected by filtration, washed with PE, and dried under vacuum to give benzyl (cyano(phenyl)methyl)glycinate hydrochloride (6.5 g, yield 46.4%) as a white solid. LC/MS (ESI) m/z: 281 (M+H)⁺.

### Step 2: Benzyl 2-(3,5-dichloro-2-oxo-6-phenylpyrazin-1(2H)-yl)acetate (3)

To a solution of benzyl (cyano(phenyl)methyl)glycinate hydrochloride (6.5 g, 23.2 mmol) in chlorobenzene (65 mL) was added oxalyl dichloride (12.0 g, 92.9 mmol) under N₂ atmosphere and the reaction mixture was stirred at 100° C. for 16 hours. The mixture was concentrated under reduced pressure to dryness. The residue was purified by flash chromatography (silica gel, 0-25% EtOAc in PE) to give benzyl 2-(3,5-dichloro-2-oxo-6-phenylpyrazin-1(2H)-yl)acetate (6.17 g, yield 68.9%) as a brown solid. LC/MS (ESI) m/z: 389 (M+H)⁺.

### Step 3: Benzyl 2-(5-chloro-3-((2,4-dimethoxybenzyl)amino)-2-oxo-6-phenylpyrazin-1(2H)-yl)acetate (4)

To a solution of benzyl 2-(3,5-dichloro-2-oxo-6-phenylpyrazin-1(2H)-yl)acetate (6.0 g, 15.5 mmol) in EtOAc (60 mL) was added 2,4-dimethoxybenzylamine (3.87 g, 23.3 mmol) under N₂ atmosphere and the reaction mixture was stirred at 80° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give benzyl 2-(5-chloro-3-((2,4-dimethoxybenzyl)amino)-2-oxo-6-phenylpyrazin-1(2H)-yl)acetate (7.5 g, yield 93.8%) as a light-yellow solid. LC/MS (ESI) m/z: 520 (M+H)⁺.

### Step 4: 2-(3-((2,4-dimethoxybenzyl)amino)-2-oxo-6-phenylpyrazin-1(2H)-yl)acetic acid (5)

To a solution of benzyl 2-(5-chloro-3-((2,4-dimethoxybenzyl)amino)-2-oxo-6-phenylpyrazin-1(2H)-yl)acetate (4.7 g, 9.1 mmol) in MeOH (50 mL) was added Pd/C (200 mg, wt. 10%), the mixture was degassed under N₂ atmosphere for ten times and stirred under a H₂ balloon at 40° C. for 16 hours. The mixture was filtered, and the filtrate was concentrated under reduced pressure to dryness to give 2-(3-((2,4-dimethoxybenzyl)amino)-2-oxo-6-phenylpyrazin-1(2H)-yl)acetic acid (2.4 g, yield 66.7%) as a yellow solid, which was used directly in the next step without further purification. LC/MS (ESI) (m/z): 396 (M+H)⁺.

### Step 5: Methyl 2-(3-((2,4-dimethoxybenzyl)amino)-2-oxo-6-phenylpyrazin-1(2H)-yl)acetate (6)

To a solution of 2-(3-((2,4-dimethoxybenzyl)amino)-2-oxo-6-phenylpyrazin-1(2H)-yl)acetic acid (2.5 g, 6.33 mmol) in THF (30 mL) was added TMSCHN₂ (4.1 mL, 8.23 mmol) at 0° C. under N₂ atmosphere and the reaction mixture was stirred at room temperature for 2 hours. The mixture was quenched with ice-water and extracted with EtOAc twice. The combined organic layers were washed with water and brine, dried over anhydrous Na₂SO₄, filtered, and concentrated under reduced pressure to dryness. The residue was purified by flash chromatography (silica gel, 0-30% EtOAc in PE) to give methyl 2-(3-((2,4-dimethoxybenzyl)amino)-2-oxo-6-phenylpyrazin-1(2H)-yl)acetate (850 mg, yield 32.7%) as a brown solid. LC/MS (ESI) m/z: 410 (M+H)⁺.

### Step 6: Methyl 2-(3-amino-2-oxo-6-phenylpyrazin-1(2H)-yl)acetate (7)

To a solution of methyl 2-(3-((2,4-dimethoxybenzyl)amino)-2-oxo-6-phenylpyrazin-1(2H)-yl)acetate (850 mg, 2.08 mmol) in DCM (1 mL) was added TFA (10 mL), and the mixture was stirred at room temperature for 16 hours. The mixture was concentrated to dryness and the residue was dissolved in EtOAc. The mixture was washed saturated aq. NaHCO₃ solution and brine, dried over anhydrous Na₂SO₄, filtered, and concentrated under reduced pressure to dryness to give methyl 2-(3-amino-2-oxo-6-phenylpyrazin-1(2H)-yl)acetate (520 mg, yield 96.7%) as a brown solid. LC/MS (ESI) m/z: 260 (M+H)⁺.

### Step 7: Methyl 2-(3-(([1,1′-biphenyl]-4-ylmethyl)amino)-2-oxo-6-phenylpyrazin-1(2H)-yl)acetate (8)

To a solution of methyl 2-(3-amino-2-oxo-6-phenylpyrazin-1(2H)-yl)acetate (80 mg, 0.31 mmol) in DCE (3 mL) was added [1,1′-biphenyl]-4-carbaldehyde (224 mg, 1.24 mmol) and AcOH (0.05 mL) under N₂ atmosphere and the reaction mixture was stirred at 80° C. for 16 hours. The mixture was cooled to 0° C. and NaBH(OAc)₃ (327 mg, 1.55 mmol) was added. The resulting mixture was stirred at 0° C. to r.t. for 1 hour. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give methyl 2-(3-(([1,1′-biphenyl]-4-ylmethyl)amino)-2-oxo-6-phenylpyrazin-1(2H)-yl) acetate (28 mg, yield 21.4%) as a white solid. LC/MS (ESI) m/z: 426 (M+H)⁺.

### Step 8: 2-(3-(([1,1′-biphenyl]-4-ylmethyl)amino)-2-oxo-6-phenylpyrazin-1(2H)-yl) acetic acid (9)

To a solution of methyl 2-(3-(([1,1′-biphenyl]-4-ylmethyl)amino)-2-oxo-6-phenylpyrazin-1(2H)-yl)acetate (28 mg, 0.07 mmol) in MeOH (3 mL) and water (1 mL) was added LiOH·H₂O (4.4 mg, 0.11 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give 2-(3-(([1,1′-biphenyl]-4-ylmethyl)amino)-2-oxo-6-phenylpyrazin-1(2H)-yl)acetic acid (25 mg, yield 92.6%) as a white solid, which was used directly in the next step without further purification. LC/MS (ESI) m/z: 412 (M+H)⁺.

### Step 9: N-((1H-pyrrolo[3,2-c]pyridin-2-yl)methyl)-2-(3-(([1,1′-biphenyl]-4-ylmethyl) amino)-2-oxo-6-phenylpyrazin-1(2H)-yl)acetamide (Compound 60)

To a mixture of 2-(3-(([1,1′-biphenyl]-4-ylmethyl)amino)-2-oxo-6-phenylpyrazin-1(2H)-yl)acetic acid (25 mg, 0.06 mmol) and (1H-pyrrolo[3,2-c]pyridin-2-yl)methanamine hydrochloride (18 mg, 0.12 mmol) in DMF (2 mL) was added DIPEA (47 mg, 0.36 mmol) and HATU (34 mg, 0.09 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-HPLC to give Compound 60 (3 mg, yield 9.1%) as a white solid. ¹H NMR (400 MHz, DMSO-d₆) δ 11.31 (s, 1H), 8.74 (s, 1H), 8.64 (s, 1H), 8.12 (d, J=5.2 Hz, 1H), 7.90 (s, 1H), 7.62 (dd, J=13.2, 8.0 Hz, 4H), 7.45 (t, J=8.3 Hz, 5H), 7.35 (d, J=9.3 Hz, 6H), 6.66 (s, 1H), 6.31 (s, 1H), 4.58 (d, J=5.3 Hz, 2H), 4.41 (s, 4H). LC/MS (ESI) m/z: 541 (M+H)⁺. RT (Method A): 1.81 min.

### Scheme 20. Synthesis of (R)—N-((1H-pyrrolo[3,2-c]pyridine-2-yl)methyl)-2-(6-oxo-5-((1-(4-phenoxyphenyl)ethyl)amino)-2-phenylpyrimidin-1(6H)-yl)acetamide (Compound 61)

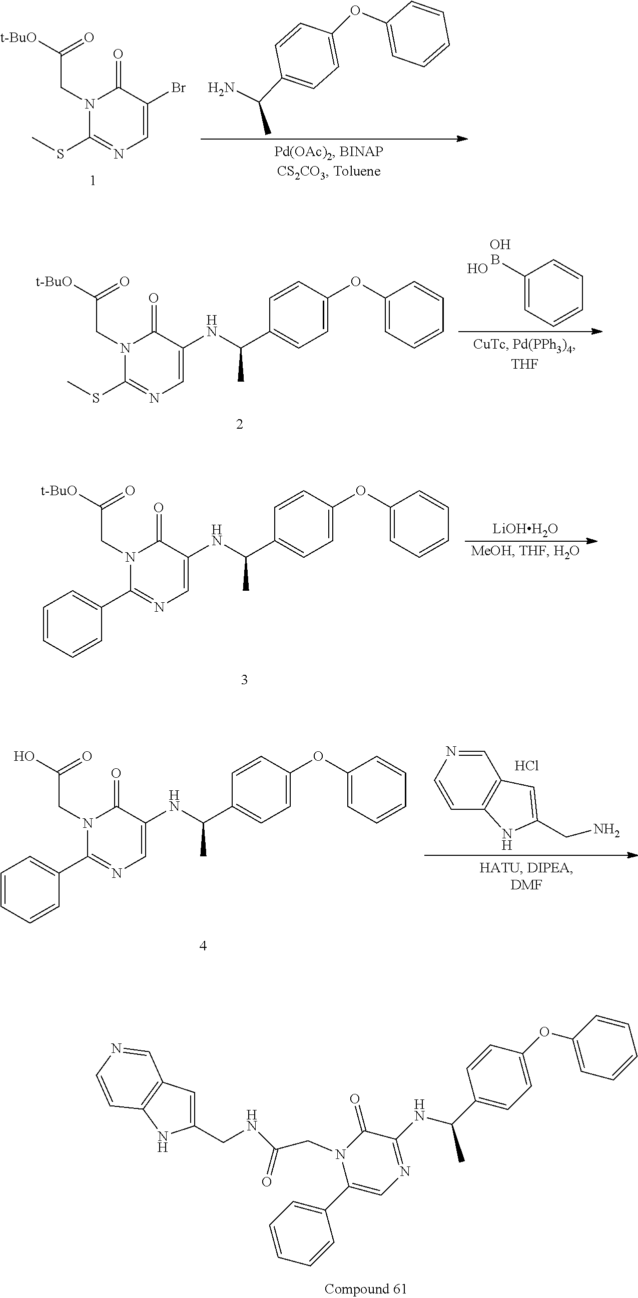

### Step 1: Tert-butyl (R)-2-(2-(methylthio)-6-oxo-5-((1-(4-phenoxyphenyl)ethyl) amino)pyrimidin-1(6H)-yl)acetate (2)

To a mixture of tert-butyl 2-(5-bromo-2-(methylthio)-6-oxopyrimidin-1(6H)-yl)acetate (960 mg, 2.87 mmol) and (R)-1-(4-phenoxyphenyl)ethan-1-amine (1.16 g, 5.44 mmol) in toluene (20 mL) was added Pd(OAc)₂ (65 mg, 0.29 mmol), Cs₂CO₃ (1.89 g, 5.75 mmol) and BINAP (360 mg, 0.58 mmol) under N₂ atmosphere at 0° C. and the mixture was stirred at 120° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (750 mg, yield 55.9%) as a colorless oil. LC/MS (ESI) (m/z): 468 (M+H)⁺.

### Step 2: Tert-butyl 2-(6-oxo-5-((1-(4-phenoxyphenyl)ethyl)amino)-2-phenylpyrimidin-1(6H)-yl)acetate (3)

To a mixture of tert-butyl I-2-(2-(methylthio)-6-oxo-5-((1-(4-phenoxyphenyl)ethyl) amino)pyrimidin-1(6H)-yl)acetate (400 mg, 0.86 mmol) and phenylboronic acid (209 mg, 1.71 mmol) in THF (5 mL) was added CuTc (360 mg, 1.88 mmol) and Pd(PPh₃)₄ (199 mg, 0.17 mmol) at room temperature and the mixture was stirred at 80° C. under N₂ atmosphere for 2 hours. The mixture was quenched with saturated aq. NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (230 mg, yield 54.1%) as a white solid. LC/MS (ESI) m/z: 498 (M+H)⁺.

### Step 3: 2-(6-oxo-5-((1-(4-phenoxyphenyl)ethyl)amino)-2-phenylpyrimidin-1(6H)-yl)acetic acid (4)

To a solution of tert-butyl I-2-(6-oxo-5-((1-(4-phenoxyphenyl)ethyl)amino)-2-phenylpyrimidin-1(6H)-yl)acetate (220 mg, 0.44 mmol) in MeOH/THF/H₂O (2 mL, 2/1/1) was added LiOH·H₂O (56 mg, 1.32 mmol) under N₂ atmosphere and the reaction mixture was stirred at room temperature for 2 hours. The mixture was acidified with 1N aq. HCl to pH˜3 and extracted with EtOAc twice. The combined organic layers were dried over anhydrous Na₂SO₄, filtered, and concentrated under reduced pressure to give the title compound (153 mg, yield 77%) as a white solid, which was used directly in the next step without further purification. LC/MS (ESI) m/z: 442 (M+H)⁺.

### Step 4: (R)—N-((1H-pyrrolo[3,2-c]pyridine-2-yl)methyl)-2-(6-oxo-5-((1-(4-phenoxyphenyl)ethyl)amino)-2-phenylpyrimidin-1(6H)-yl)acetamide (Compound 61)

To a mixture of p2-(6-oxo-5-((1-(4-phenoxyphenyl)ethyl)amino)-2-phenylpyrimidin-1(6H)-yl)acetic acid (60 mg, 0.07 mmol) and (1H-pyrrolo[3,2-c]pyridine-2-yl)methanamine (12 mg, 0.08 mmol) in DMF (1 mL) was added DIPEA (53 mg, 0.40 mmol) and HATU (34 mg, 0.09 mmol) at 0° C. under N₂ atmosphere and the mixture was stirred at room temperature for 2 hour. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by prep-TLC (DCM:MeOH=10:1) and further purified by prep-HPLC to give Compound 61 (1.8 mg, yield 2.3%) as a white solid. ¹H NMR (400 MHz, CD₃OD) δ 8.75 (s, 1H), 8.13 (d, J=5.3 Hz, 1H), 7.43 (d, J=6.4 Hz, 1H), 7.37 (d, J=7.0 Hz, 1H), 7.33 (s, 1H), 7.09 (s, 1H), 6.96 (s, 1H), 6.88 (s, 1H), 6.52 (s, 1H), 4.62 (s, 1H), 4.55 (s, 1H), 4.50 (d, J=5.1 Hz, 1H), 1.59 (d, J=5.9 Hz, 1H). LC/MS (ESI) m/z: 571 (M+H)⁺. RT (Method A): 1.76 min.

### Scheme 21. Synthesis of N-((1H-pyrrolo[3,2-c]pyridine-2-yl)methyl)-2-(2-oxo-3-((4-phenoxybenzyl)amino)-6-phenylpyrazin-1(2H)-yl)acetamide (Compound 63)

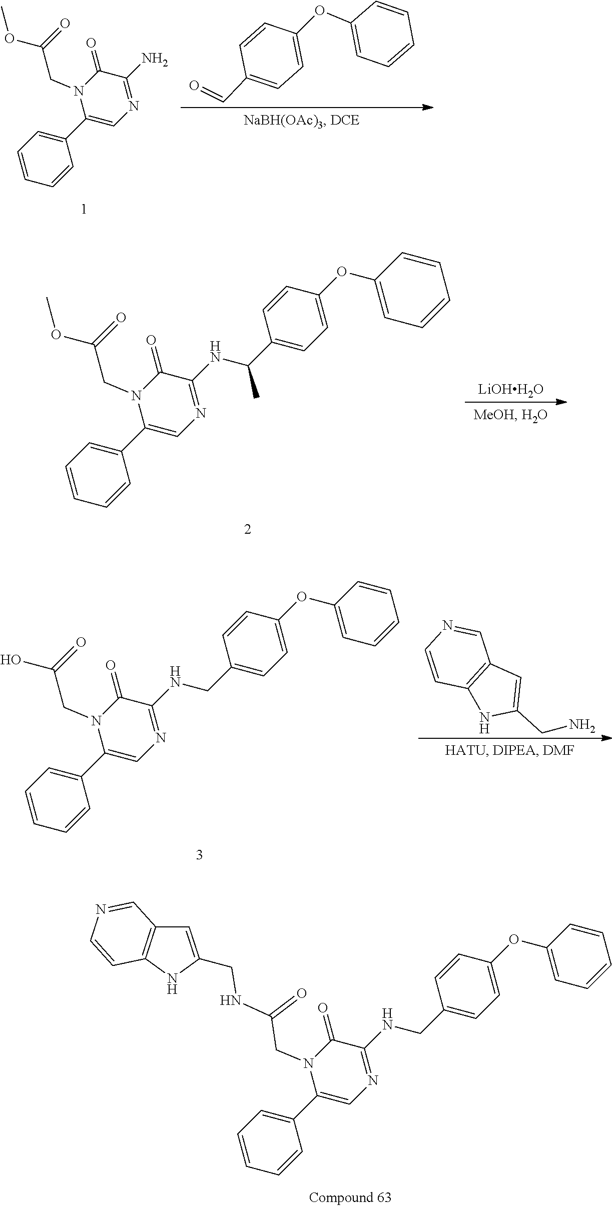

### Step 1: Methyl 2-(2-oxo-3-((4-phenoxybenzyl)amino)-6-phenylpyrazin-1(2H)-yl)acetate (2)

To a solution of methyl 2-(3-amino-2-oxo-6-phenylpyrazin-1(2H)-yl)acetate (80 mg, 0.31 mmol) in DCE (3 mL) was added 4-phenoxybenzaldehyde (198 mg, 1.24 mmol) and AcOH (0.05 mL) under N₂ atmosphere and the reaction mixture was stirred at 80° C. for 16 hours. NaBH(OAc)₃ (327 mg, 1.55 mmol) was added to the mixture and the resulting mixture was stirred at 80° C. for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-25% EtOAc in PE) to give the title compound (18 mg, yield 13.2%) as a white solid. LC/MS (ESI) m/z: 442 (M+H)⁺.

### Step 2: 2-(2-oxo-3-((4-phenoxybenzyl)amino)-6-phenylpyrazin-1(2H)-yl)acetic acid (3)

To a solution of methyl 2-(2-oxo-3-((4-phenoxybenzyl)amino)-6-phenylpyrazin-1(2H)-yl)acetate (18 mg, 0.04 mmol) in MeOH (2.1 mL) and water (0.7 mL) was added LiOH·H₂O (2.6 mg, 0.06 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (17 mg, yield 97.7%) as a yellow oil, which was used directly in the next step without further purification. LC/MS (ESI) m/z: 428 (M+H)⁺.

### Step 3: N-((1H-pyrrolo[3,2-c]pyridine-2-yl)methyl)-2-(2-oxo-3-((4-phenoxybenzyl) amino)-6-phenylpyrazin-1(2H)-yl)acetamide (Compound 63)

To a mixture of 2-(2-oxo-3-((4-phenoxybenzyl)amino)-6-phenylpyrazin-1(2H)-yl)acetic acid (20 mg, 0.05 mmol) and (1H-pyrrolo[3,2-c]pyridine-2-yl)methanamine hydrochloride (14 mg, 0.10 mmol) in DMF (2 mL) was added DIPEA (36 mg, 0.30 mmol) and HATU (27 mg, 0.08 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-HPLC to give Compound 63 (1.0 mg, yield 3.8%) as a white solid. ¹H NMR (400 MHz, CD₃OD) δ 8.90 (s, 1H), 8.46 (s, 1H), 8.22 (d, J=4.9 Hz, 1H), 7.69 (d, J=6.3 Hz, 1H), 7.41-7.30 (m, 8H), 7.09 (t, J=7.9 Hz, 1H), 6.97 (dd, J=19.3, 12.1 Hz, 4H), 6.73 (d, J=16.3 Hz, 2H), 4.62 (s, 2H), 4.58 (s, 2H), 4.56 (s, 2H). LC/MS (ESI) m/z: 557 (M+H)⁺. RT (Method A): 1.27 min.

### Scheme 22. Synthesis of N-((1H-pyrrolo[3,2-c]pyridine-2-yl)methyl)-2-(5-((3-(4-(fluoromethoxy)phenyl)propyl)amino)-6-oxo-2-phenylpyrimidin-1(6H)-yl)acetamide (Compound 66)

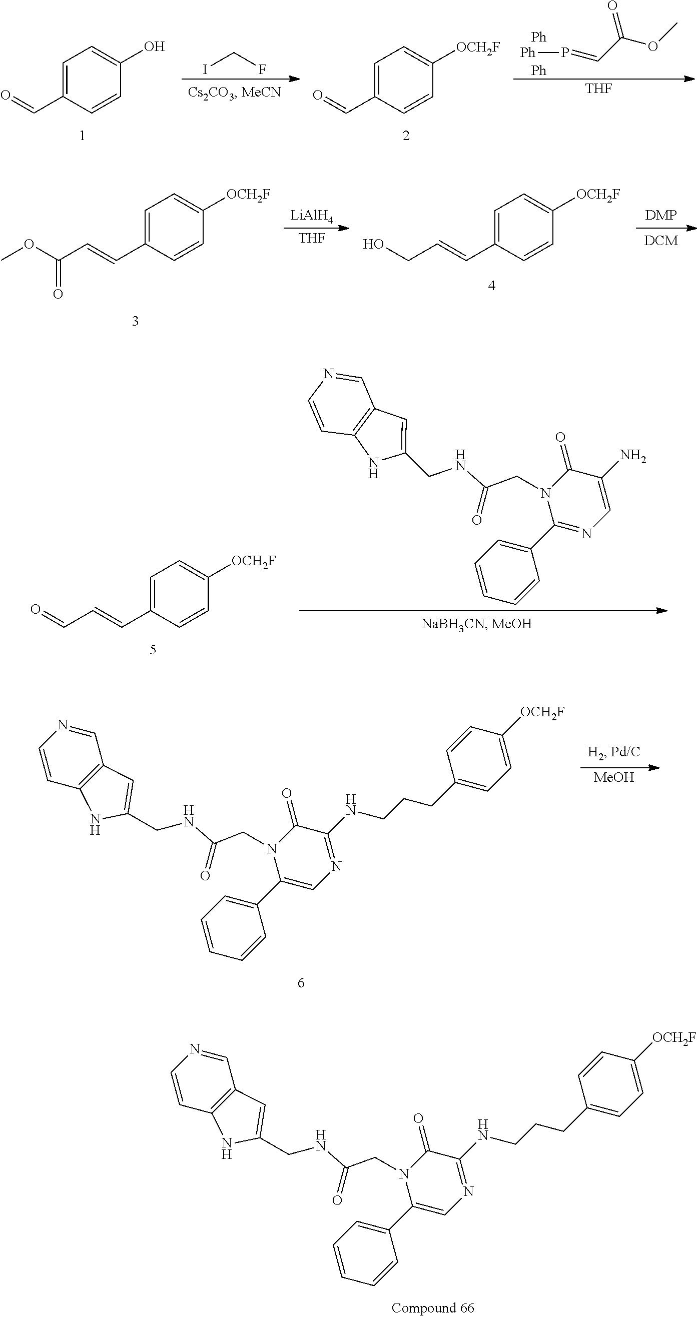

### Step 1: 4-(Fluoromethoxy)benzaldehyde (2)

To a mixture of 4-hydroxybenzaldehyde (700 mg, 5.73 mmol) and fluoroiodomethane (1.00 g, 6.25 mmol) in MeCN (12 mL) was added Cs₂CO₃ (2.24 g, 6.88 mmol) and the mixture was stirred in a sealed tube at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-20% EtOAc in PE) to give the title compound (880 mg, yield 99.6%) as a light-yellow oil. ¹H NMR (400 MHz, CDCl₃) δ 9.94 (s, 1H), 7.92-7.85 (m, 2H), 7.20 (d, J=8.6 Hz, 2H), 5.85 (s, 1H), 5.72 (s, 1H). LC/MS (ESI) m/z: 155 (M+H)⁺.

### Step 2: Methyl (E)-3-(4-(fluoromethoxy)phenyl)acrylate (3)

To a solution of 4-(fluoromethoxy)benzaldehyde (750 mg, 4.87 mmol) in THF (15 mL) was added methyl 2-(triphenyl-λ⁵-phosphaneylidene)acetate (4.83 g, 14.44 mmol) under N₂ atmosphere and the mixture was stirred at 60° C. overnight. The mixture was concentrated under reduced pressure to dryness. The residue was purified by flash chromatography (silica gel, 0-20% EtOAc in PE) to give the title compound (770 mg, yield 75.3%) as a white solid. ¹H NMR (400 MHz, CDCl₃) δ 7.65 (d, J=15.8 Hz, 1H), 7.50 (d, J=7.9 Hz, 2H), 7.08 (d, J=7.9 Hz, 2H), 6.35 (d, J=16.0 Hz, 1H), 5.80 (s, 1H), 5.66 (s, 1H), 3.80 (s, 3H). LC/MS (ESI) m/z: 211 (M+H)⁺.

### Step 3: (E)-3-(4-(fluoromethoxy)phenyl)prop-2-en-1-ol (4)

To a solution of methyl (E)-3-(4-(fluoromethoxy)phenyl)acrylate (400 mg, 1.90 mmol) in THF (8 mL) was added LiAlH₄ (9.5 mL, 9.50 mmol, 1 M) drop-wisely at −20° C. under N₂ atmosphere and the reaction mixture was stirred at −20° C. for 2 hours. The mixture was quenched with Na₂SO₄·10H₂O at 0° C. and the mixture was stirred at room temperature for 10 mins. The mixture was filtered, and the filtrate was dried over anhydrous Na₂SO₄, filtered, and concentrated under reduced pressure to dryness. The residue was purified by flash chromatography (silica gel, 0-30% EtOAc in PE) to give the title compound (230 mg, yield 66.3%) as a white solid. ¹H NMR (400 MHz, CDCl₃) δ 7.35 (d, J=7.4 Hz, 2H), 7.03 (d, J=7.5 Hz, 2H), 6.58 (d, J=15.8 Hz, 1H), 6.30 (s, 1H), 5.77 (s, 1H), 5.64 (s, 1H), 4.31 (s, 2H). LC/MS (ESI) m/z: 165 (M-OH)⁺.

### Step 4: (E)-3-(4-(fluoromethoxy)phenyl)acrylaldehyde (5)

To a solution of (E)-3-(4-(fluoromethoxy)phenyl)prop-2-en-1-ol (230 mg, 1.26 mmol) in DCM (10 mL) was added Dess-Martin periodinane (1.49 g, 3.51 mmol) and NaHCO₃ (294 mg, 3.50 mmol) at 0° C., the reaction mixture was stirred at room temperature for 1 hour. The mixture was diluted with DCM, washed with water and brine, dried over anhydrous Na₂SO₄, filtered, and concentrated under reduced pressure to dryness. The residue was purified by flash chromatography (silica gel, 0-10% EtOAc in PE) to give the title compound (123 mg, yield 54.1%) as a white solid. ¹H NMR (400 MHz, CDCl₃) δ 9.68 (d, J=7.5 Hz, 1H), 7.56 (d, J=7.8 Hz, 2H), 7.44 (d, J=16.0 Hz, 1H), 7.13 (d, J=7.8 Hz, 2H), 6.64 (dd, J=15.9, 7.6 Hz, 1H), 5.75 (d, J=54.0 Hz, 2H). LC/MS (ESI) m/z: 181 (M+H)⁺.

### Step 5: (E)-N-((1H-pyrrolo[3,2-c]pyridine-2-yl)methyl)-2-(5-((3-(4-(fluoromethoxy) phenyl)allyl)amino)-6-oxo-2-phenylpyrimidin-1(6H)-yl)acetamide (6)

To a solution of (E)-3-(4-(fluoromethoxy)phenyl)acrylaldehyde (14 mg, 0.078 mmol) in MeOH (2 mL) was added N-((1H-pyrrolo[3,2-c]pyridine-2-yl)methyl)-2-(5-amino-6-oxo-2-phenylpyrimidin-1(6H)-yl)acetamide (20 mg, 0.053 mmol) and NaBH₃CN (7 mg, 0.11 mmol) under N₂ atmosphere and the reaction mixture was stirred at 55° C. for 1 hour. Then another batch of (E)-3-(4-(fluoromethoxy)phenyl)acrylaldehyde (14 mg, 0.078 mmol) and NaBH₃CN (7 mg, 0.11 mmol) were added to the mixture and the resulting mixture was stirred at 55° C. overnight. The mixture was quenched with saturated aq. NH₄Cl solution and extracted with DCM twice. The combined organic layers were washed with brine, dried over anhydrous Na₂SO₄, filtered, and concentrated under reduced pressure to dryness. The residue was purified by prep-TLC (DCM:MeOH=15:1) to give the title compound (15 mg, yield 52.1%) as a light-yellow oil. LC/MS (ESI) m/z: 539 (M+H)⁺.

### Step 6: N-((1H-pyrrolo[3,2-c]pyridine-2-yl)methyl)-2-(5-((3-(4-(fluoromethoxy)phenyl) propyl)amino)-6-oxo-2-phenylpyrimidin-1(6H)-yl)acetamide (Compound 66)

To a solution of (E)-N-((1H-pyrrolo[3,2-c]pyridine-2-yl)methyl)-2-(5-((3-(4-(fluoromethoxy)phenyl)allyl)amino)-6-oxo-2-phenylpyrimidin-1(6H)-yl)acetamide (15 mg, 0.028 mmol) in MeOH (2 mL) was added Pd/C (5 mg, 10% wt.), the mixture was degassed under N₂ atmosphere for three times and stirred under a H₂ balloon at room temperature for 10 minutes. The mixture was filtered, and the filtrate was concentrated to dryness. The residue was purified by prep-HPLC to give Compound 66 (0.76 mg, yield 5.0%) as a white solid. ¹H NMR (400 MHz, CD₃OD) δ 8.78 (s, 1H), 8.15 (d, J=6.0 Hz, 1H), 7.50-7.45 (m, 4H), 7.42-7.38 (m, 2H), 7.21 (d, J=8.5 Hz, 2H), 7.09 (s, 1H), 7.03-6.99 (m, 2H), 6.57 (s, 1H), 5.77 (s, 1H), 5.64 (s, 1H), 4.63 (s, 2H), 4.56 (s, 2H), 3.19 (t, J=6.9 Hz, 2H), 2.73 (t, J=7.6 Hz, 2H), 2.01-1.95 (m, 2H). LC/MS (ESI) m/z: 541 (M+H)⁺. RT (Method a): 1.49 min.

### Scheme 23. Synthesis of N-((1H-pyrrolo[3,2-c]pyridine-2-yl)methyl)-2-(6-oxo-5-((3-phenylpropyl)amino)-2-(4-(sulfamoylmethoxy)phenyl)pyrimidin-1(6H)-yl)acetamide (Compound 67)

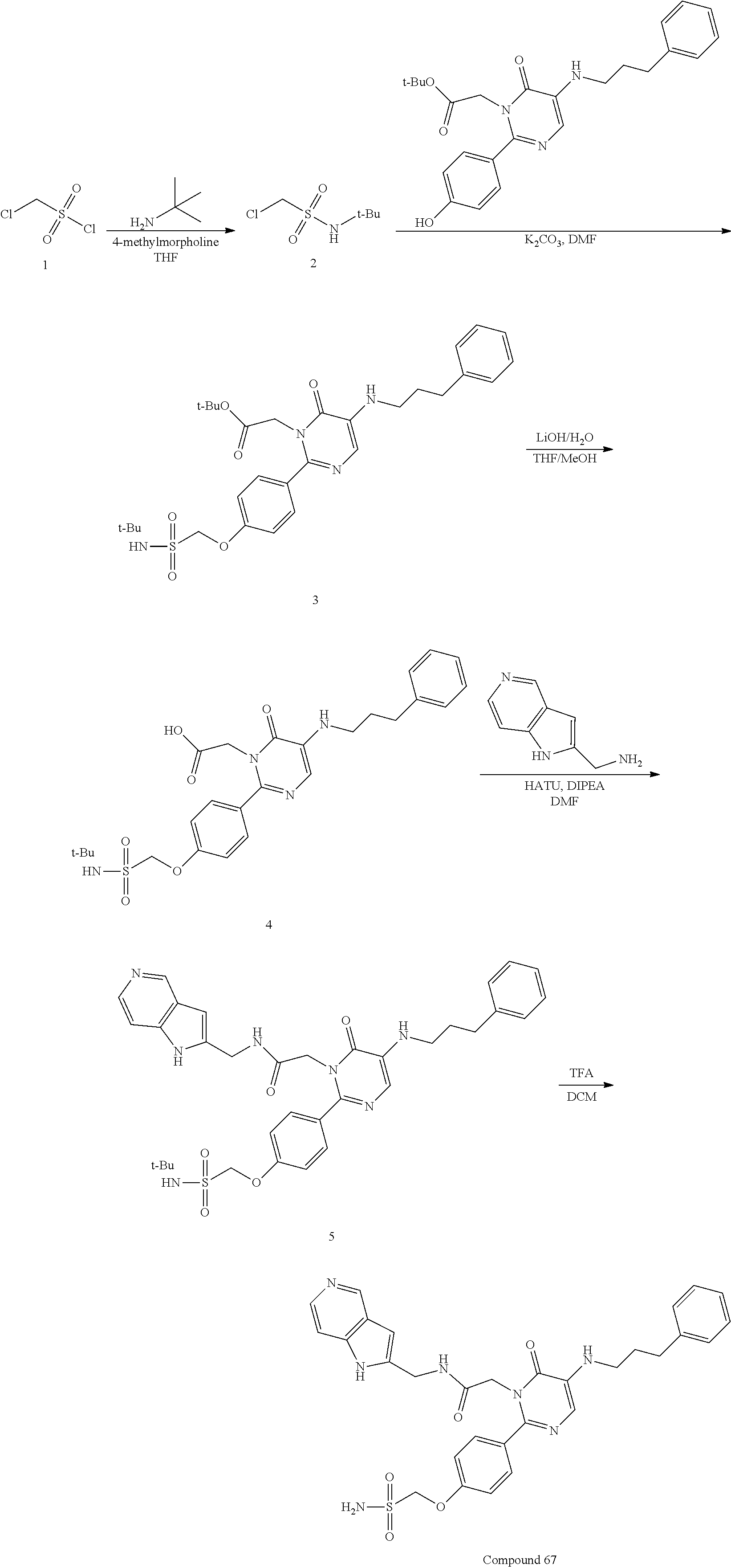

### Step 1: N-(tert-butyl)-1-chloromethanesulfonamide (2)

To a mixture of 4-methylmorpholine (375 mg, 3.70 mmol) and 2-methylpropan-2-amine (271 mg, 3.70 mmol) in THF (5 mL) was added chloromethanesulfonyl chloride (500 mg, 3.37 mmol) at 0° C. and the mixture was stirred at room temperature for 2 hours. The mixture was diluted with EtOAc, washed with 1 M aq. HCl and brine, dried over anhydrous Na₂SO₄, filtered, and concentrated under reduced pressure to dryness to give the title compound (360 mg, yield 57.6%) as a white solid, which was used directly in the next step without further purification. LC/MS (ESI) m/z: 185 (M+H)⁺.

### Step 2: Tert-butyl 2-(2-(4-((N-(tert-butyl)sulfamoyl)methoxy)phenyl)-6-oxo-5-((3-phenylpropyl)amino)pyrimidin-1(6H)-yl)acetate (3)

To a solution of tert-butyl 2-(2-(4-hydroxyphenyl)-6-oxo-5-((3-phenylpropyl)amino) pyrimidin-1(6H)-yl)acetate (150 mg, 0.34 mmol) in DMF (2 mL) was added K₂CO₃ (142 mg, 1.02 mmol) and N-(tert-butyl)-1-chloromethanesulfonamide (64 mg, 0.34 mmol) at 0° C. and the mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-60% EtOAc in PE) to give the title compound (120 mg, yield 59.7%) as a yellow solid. LC/MS (ESI) m/z: 585 (M+H).

### Step 3: 2-(2-(4-((N-(tert-butyl)sulfamoyl)methoxy)phenyl)-6-oxo-5-((3-phenylpropyl) amino)pyrimidin-1(6H)-yl)acetic acid (4)

To a solution of tert-butyl 2-(2-(4-((N-(tert-butyl)sulfamoyl)methoxy)phenyl)-6-oxo-5-((3-phenylpropyl)amino)pyrimidin-1(6H)-yl)acetate (120 mg, 0.20 mmol) in MeOH (1 mL), THF (1 mL) and water (1 mL) was added NaOH (16 mg, 0.40 mmol) and the mixture was stirred at 40° C. for 2 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (88 mg, yield 81.2%) as a yellow solid, which was used directly in the next step without further purification. LC/MS (ESI) m/z: 529 (M+H)⁺.

### Step 4: N-((1H-pyrrolo[3,2-c]pyridine-2-yl)methyl)-2-(2-(4-((N-(tert-butyl)sulfamoyl) methoxy)phenyl)-6-oxo-5-((3-phenylpropyl)amino)pyrimidin-1(6H)-yl)acetamide (5)

To a mixture of 2-(2-(4-((N-(tert-butyl)sulfamoyl)methoxy)phenyl)-6-oxo-5-((3-phenylpropyl)amino)pyrimidin-1(6H)-yl)acetic acid (88 mg, 0.16 mmol) and (1H-pyrrolo[3,2-c]pyridine-2-yl)methanamine (73 mg, 0.48 mmol) in DMF (2 mL) was added DIPEA (107 mg, 0.80 mmol) and HATU (95 mg, 0.24 mmol) at 0° C. and the mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=10:1) to give the title compound (70 mg, yield 64.2%) as a yellow solid. LC/MS (ESI) m/z: 658 (M+H)⁺.

### Step 5: N-((1H-pyrrolo[3,2-c]pyridine-2-yl)methyl)-2-(6-oxo-5-((3-phenylpropyl) amino)-2-(4-(sulfamoylmethoxy)phenyl)pyrimidin-1(6H)-yl)acetamide (Compound 67)

To a solution of N-((1H-pyrrolo[3,2-c]pyridine-2-yl)methyl)-2-(2-(4-((N-(tert-butyl)sulfamoyl)methoxy)phenyl)-6-oxo-5-((3-phenylpropyl)amino)pyrimidin-1(6H)-yl)acetamide (70 mg, 0.10 mmol) in DCM (1 mL) was added TFA (0.5 mL) and the mixture was stirred under N₂ atmosphere at room temperature for 1 hour. The reaction mixture was concentrated under reduced pressure to dryness. The residue was purified by prep-HPLC to give Compound 67 (13.9 mg, yield 21.7%) as a white solid. ¹H NMR (400 MHz, DMSO-d₆) δ 11.32 (s, 1H), 8.74 (s, 1H), 8.70 (t, J=5.7 Hz, 1H), 8.11 (d, J=5.7 Hz, 1H), 7.43 (d, J=8.7 Hz, 2H), 7.35-7.26 (m, 4H), 7.24 (d, J=6.7 Hz, 3H), 7.20-7.17 (m, 1H), 7.13 (d, J=8.8 Hz, 2H), 7.08 (s, 1H), 6.36 (s, 1H), 5.48 (t, J=5.9 Hz, 1H), 5.11 (s, 2H), 4.50 (s, 2H), 4.43 (d, J=5.4 Hz, 2H), 3.12-3.06 (m, 2H), 2.67 (t, J=7.6 Hz, 2H), 1.93-1.85 (m, 2H). LC/MS (ESI) m/z: 602 (M+H)⁺. RT (Method A): 1.32 min.

Compounds 131 and 139 are prepared based on the procedures set forth in Scheme 23.

| # | Reactant A | Reactant B | Reactant C | Reactant D |
|---|---|---|---|---|
| 131 | 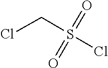 | 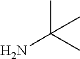 | 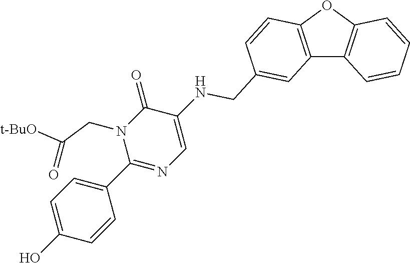 [SMILES: CC(C)(C)OC(=O)Cn1c(-c2ccc(O)cc2)ncc(NCc2ccc3oc4ccccc4c3c2)c1=O] | 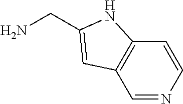 [SMILES: NCc1cc2cnccc2[nH]1] |
| 139 | 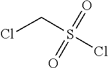 | 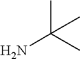 | 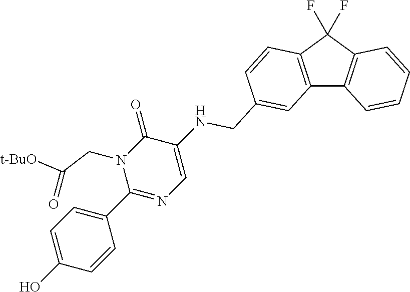 [SMILES: CC(C)(C)OC(=O)Cn1c(-c2ccc(O)cc2)ncc(NCc2ccc3c(c2)-c2ccccc2C3(F)F)c1=O] | 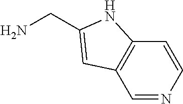 [SMILES: NCc1cc2cnccc2[nH]1] |

### Scheme 24. Synthesis of (4-(1-(2-(((1H-pyrrolo[3,2-c]pyridine-2-yl)methyl)amino)-2-oxoethyl)-6-oxo-5-((3-phenylpropyl)amino)-1,6-dihydropyrimidin-2-yl)phenyl)(methyl) phosphinic acid (Compound 68)

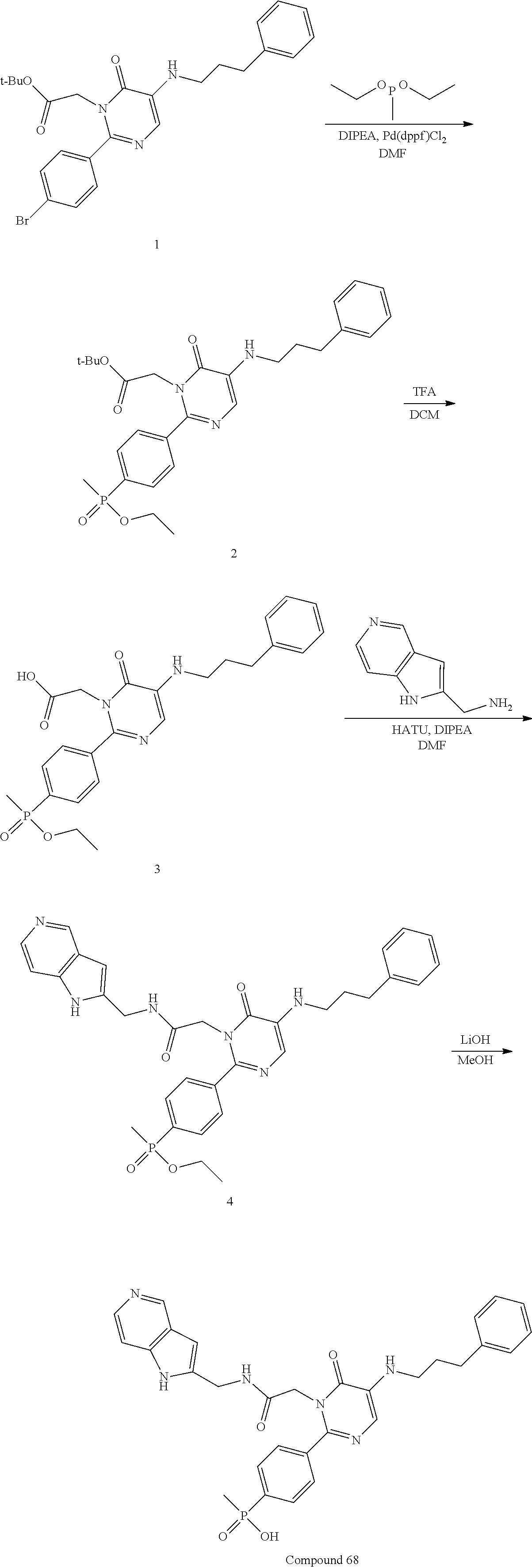

### Step 1: Tert-butyl 2-(2-(4-(ethoxy(methyl)phosphoryl)phenyl)-6-oxo-5-((3-phenylpropyl)amino)pyrimidin-1(6H)-yl)acetate (2)

To a mixture of tert-butyl 2-(2-(4-bromophenyl)-6-oxo-5-((3-phenylpropyl)amino) pyrimidin-1(6H)-yl)acetate (120 mg, 0.24 mmol) and diethyl methylphosphonite (164 mg, 1.20 mmol) in DMF (3 mL) was added DIPEA (156 mg, 1.2 mmol) followed by Pd(dppf)Cl₂ (17 mg, 0.02 mmol) and the reaction mixture was degassed under N₂ atmosphere for three times and stirred in a CEM microwave reactor at 130° C. for 30 mins. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-90% EtOAc in PE) to give the title compound (75 mg, yield 59.3%) as a yellow oil. LC/MS (ESI) m/z: 526 (M+H)⁺.

### Step 2: 2-(2-(4-(Ethoxy(methyl)phosphoryl)phenyl)-6-oxo-5-((3-phenylpropyl)amino) pyrimidin-1(6H)-yl)acetic acid (3)

To a solution of tert-butyl 2-(2-(4-(ethoxy(methyl)phosphoryl)phenyl)-6-oxo-5-((3-phenylpropyl)amino)pyrimidin-1(6H)-yl)acetate (75 mg, 0.14 mmol) in DCM (3 mL)) was added TFA (1 mL) and the reaction mixture was stirred at room temperature for 6 hours. The reaction mixture was concentrated under reduced pressure to dryness to give the title compound (80 mg, yield 97.8%) as a yellow solid, which was used directly in the next step without further purification. LC/MS (ESI) m/z: 470 (M+H)⁺.

### Step 3: Ethyl (4-(1-(2-(((1H-pyrrolo[3,2-c]pyridine-2-yl)methyl)amino)-2-oxoethyl)-6-oxo-5-((3-phenylpropyl)amino)-1,6-dihydropyrimidin-2-yl)phenyl)(methyl) phosphinate (4)

To a mixture of 2-(2-(4-(ethoxy(methyl)phosphoryl)phenyl)-6-oxo-5-((3-phenylpropyl) amino)pyrimidin-1(6H)-yl)acetic acid (50 mg, 0.106 mol) and (1H-pyrrolo[3,2-c]pyridine-2-yl)methanamine (31 mg, 0.21 mmol) in DMF (2 mL) was added DIPEA (41 mg, 0.32 mmol) and HATU (61 mg, 0.16 mol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-100% EtOAc in PE) to give the title compound (45 mg, yield 70.6%) as a yellow oil. LC/MS (ESI) m/z: 599 (M+H)⁺.

### Step 4: (4-(1-(2-(((1H-pyrrolo[3,2-c]pyridine-2-yl)methyl)amino)-2-oxoethyl)-6-oxo-5-((3-phenylpropyl)amino)-1,6-dihydropyrimidin-2-yl)phenyl)(methyl)phosphinic acid (Compound 68)

To a solution of ethyl (4-(1-(2-(((1H-pyrrolo[3,2-c]pyridine-2-yl)methyl)amino)-2-oxoethyl)-6-oxo-5-((3-phenylpropyl)amino)-1,6-dihydropyrimidin-2-yl)phenyl)(methyl) phosphinate (45 mg, 0.08 mmol) in MeOH (2 mL) and water (0.5 mL) was added LiOH·H₂O (6 mg, 0.15 mmol) and the mixture was stirred at room temperature for 2 hours. The mixture was purified by prep-HPLC to give Compound 68 (15 mg, yield 35.0%) as a white solid. ¹H NMR (400 MHz, DMSO) δ 12.29-11.96 (m, 1H), 9.30-8.83 (m, 1H), 8.74 (s, 1H), 8.09 (d, J=5.7 Hz, 1H), 7.69 (d, J=7.6 Hz, 2H), 7.40-7.32 (m, 3H), 7.32-7.27 (m, 2H), 7.25 (d, J=6.9 Hz, 2H), 7.21-7.16 (m, 1H), 7.08 (s, 1H), 6.34 (s, 1H), 5.47 (t, J=5.8 Hz, 1H), 4.51 (s, 2H), 4.36 (s, 2H), 3.10 (dd, J=13.1, 6.6 Hz, 2H), 2.67 (t, J=7.7 Hz, 2H), 1.94-1.86 (m, 2H), 1.16-1.08 (m, 3H). LC/MS (ESI) m/z: 571 (M+H)⁺. RT (Method A): 1.18 min.

Compounds 132 and 140 are prepared based on the procedures set forth in Scheme 24.

| # | Reactant A | Reactant B | Reactant C |
|---|---|---|---|
| 132 | 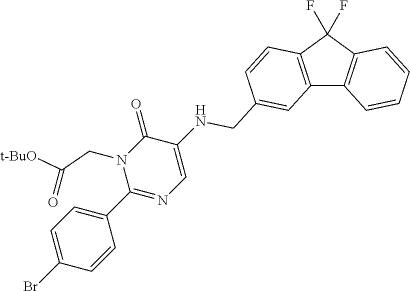 [SMILES: CC(C)(C)OC(=O)Cn1c(-c2ccc(Br)cc2)ncc(NCc2ccc3c(c2)-c2ccccc2C3(F)F)c1=O] | 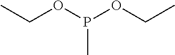 | 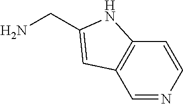 [SMILES: NCc1cc2cnccc2[nH]1] |
| 140 | 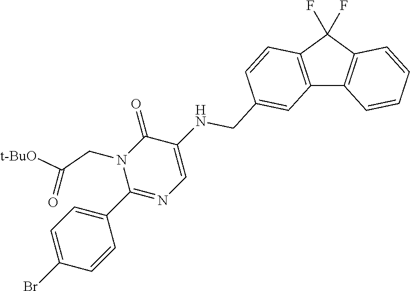 [SMILES: CC(C)(C)OC(=O)Cn1c(-c2ccc(Br)cc2)ncc(NCc2ccc3c(c2)-c2ccccc2C3(F)F)c1=O] | 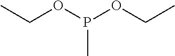 | 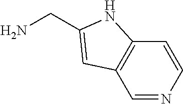 [SMILES: NCc1cc2cnccc2[nH]1] |

### Scheme 25. Synthesis of N-((1H-pyrrolo[3,2-c]pyridine-2-yl)methyl)-2-(2-(4-(methylsulfonyl) phenyl)-6-oxo-5-((3-phenylpropyl)amino)pyrimidin-1(6H)-yl)acetamide_(Compound 69)

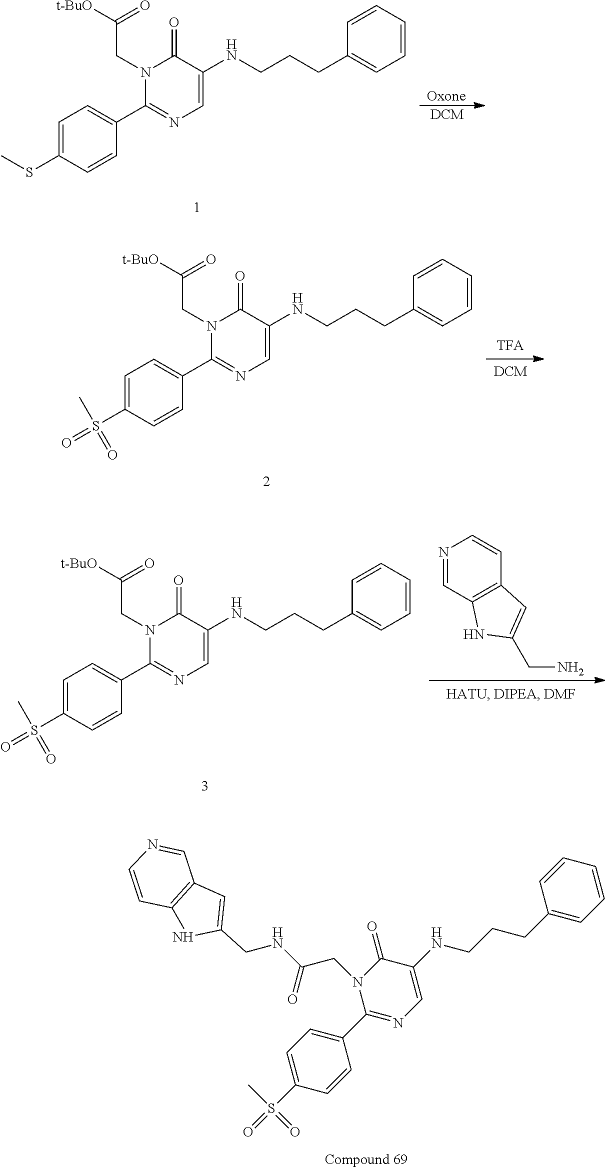

### Step 1: Tert-butyl 2-(2-(4-(methylsulfonyl)phenyl)-6-oxo-5-((3-phenylpropyl)amino) pyrimidin-1(6H)-yl)acetate (2)

To a mixture of tert-butyl 2-(2-(4-(methylthio)phenyl)-6-oxo-5-((3-phenylpropyl) amino)pyrimidin-1(6H)-yl)acetate (100 mg, 0.22 mol) in DCM (2 mL) was added Oxone (396 mg, 0.64 mmol) at 0° C. and the mixture was stirred at room temperature for 1 hour. The mixture was diluted with DCM, washed with water and brine, dried over anhydrous Na₂SO₄, filtered, and concentrated under reduced pressure to dryness. The residue was purified by flash chromatography (silica gel, 0-40% EtOAc in PE) to give the title compound (70 mg, yield 62.0%) as a yellow solid. LC/MS (ESI) (m/z): 498 (M+H)⁺.

### Step 2: 2-(2-(4-(methylsulfonyl)phenyl)-6-oxo-5-((3-phenylpropyl)amino)pyrimidin-1(6H)-yl)acetic acid (3)

To a solution of tert-butyl 2-(2-(4-(methylsulfonyl)phenyl)-6-oxo-5-((3-phenylpropyl) amino)pyrimidin-1(6H)-yl)acetate (60 mg, 0.12 mmol) in DCM (2 mL) was added TFA (1 mL) at 0° C. under N₂ atmosphere and the reaction mixture was stirred at room temperature for 2 hours. The mixture was concentrated under reduced pressure to dryness to give the title compound (35 mg, yield 65.6%) as a yellow solid, which was used directly in the next step without further purification. LC/MS (ESI) m/z: 442 (M+H)⁺.

### Step 3: N-((1H-pyrrolo[3,2-c]pyridine-2-yl)methyl)-2-(2-(4-(methylsulfonyl)phenyl)-6-oxo-5-((3-phenylpropyl)amino)pyrimidin-1(6H)-yl)acetamide (Compound 69)

To a mixture of 2-(2-(4-(methylsulfonyl)phenyl)-6-oxo-5-((3-phenylpropyl)amino) pyrimidin-1(6H)-yl)acetic acid (35 mg, 0.079 mol) and (1H-pyrrolo[2,3-c]pyridine-2-yl)methanamine (17 mg, 0.12 mmol) in DMF (1 mL) was added DIPEA (51 mg, 0.40 mmol) and HATU (45 mg, 0.12 mol) under N₂ atmosphere and the reaction mixture was stirred at room temperature for 30 mins. The mixture was diluted with EtOAc, washed with saturated aq. NH₄Cl solution and brine, dried over anhydrous Na₂SO₄, filtered, and concentrated under reduced pressure to dryness. The residue was purified by prep-HPLC to give Compound 69 (2.3 mg, yield 5.1%) as a white solid. ¹H NMR (400 MHz, DMSO-d₆) δ 11.32 (s, 1H), 8.76-8.71 (m, 2H), 8.11 (d, J=5.6 Hz, 1H), 7.97 (d, J=8.4 Hz, 2H), 7.75 (d, J=8.4 Hz, 2H), 7.33-7.23 (m, 5H), 7.18 (t, J=7.0 Hz, 1H), 7.12 (s, 1H), 6.33 (s, 1H), 5.69 (t, J=6.0 Hz, 1H), 4.52 (s, 2H), 4.42 (d, J=5.5 Hz, 2H), 3.24 (s, 3H), 3.15-3.10 (m, 2H), 2.67 (t, J=7.7 Hz, 2H), 1.93-1.86 (m, 2H). LC/MS (ESI) m/z: 571 (M+H)⁺. RT (Method A): 1.42 min.

### Scheme 26. Synthesis of N-((1H-pyrrolo[3,2-c]pyridine-2-yl)methyl)-2-(5-(3-(cyclohexylmethyl)ureido)-6-oxo-2-phenylpyrimidin-1(6H)-yl)acetamide (Compound 76)

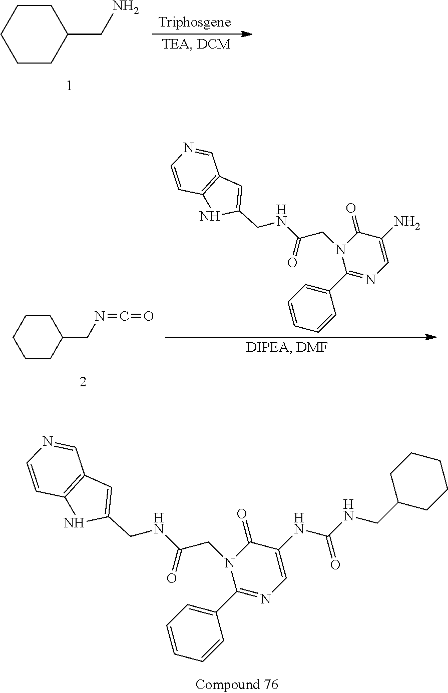

### Step 1: (Isocyanatomethyl)cyclohexane (2)

To a solution of cyclohexylmethanamine (113 mg, 1.0 mmol) in DCM (2 mL) was added a solution of bis(trichloromethyl) carbonate (297 mg, 1.0 mmol) in DCM (2 mL) and TEA (303 mg, 3.0 mmol) under N₂ atmosphere at 0° C. and the mixture was stirred at room temperature for 1 hour. The mixture was concentrated under reduced pressure to dryness to give the title compound (400 mg, crude) as a white solid, which was used directly in the next step without further purification.

### Step 2: N-((1H-pyrrolo[3,2-c]pyridine-2-yl)methyl)-2-(5-(3-(cyclohexylmethyl)ureido)-6-oxo-2-phenylpyrimidin-1(6H)-yl)acetamide (Compound 76)

To a solution of N-((1H-pyrrolo[3,2-c]pyridine-2-yl)methyl)-2-(5-amino-6-oxo-2-phenylpyrimidin-1(6H)-yl)acetamide (30 mg, 0.08 mmol) in DMF (1 mL) was added (isocyanatomethyl)cyclohexane (22 mg, 0.16 mmol) and DIPEA (16 mg, 0.12 mmol) under N₂ atmosphere and the reaction mixture was stirred at room temperature for 2 hours. The mixture was concentrated under reduced pressure to dryness. The residue was purified by prep-HPLC to give Compound 76 (2.3 mg, yield 5.6%) as a white solid. ¹H NMR (400 MHz, CD₃OD) δ 8.81 (s, 1H), 8.30 (d, J=6.0 Hz, 1H), 7.66 (d, J=6.0 Hz, 1H), 7.52-7.47 (m, 3H), 7.42 (t, J=7.4 Hz, 3H), 6.70 (s, 1H), 4.67 (s, 2H), 4.64 (s, 2H), 3.28 (d, J=6.9 Hz, 2H), 1.86 (d, J=12.5 Hz, 2H), 1.82-1.76 (m, 2H), 1.74-1.67 (m, 2H), 1.35-1.25 (m, 3H), 1.12-1.03 (m, 2H). LC/MS (ESI) m/z: 514 (M+H)⁺. RT (Method A): 1.20 min.

Compound 79 was prepared based on Step 2 in Scheme 26:

| # | Reactant A | Reactant B | Characterization Data |
|---|---|---|---|
| 79 | 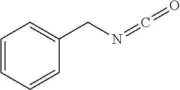 | 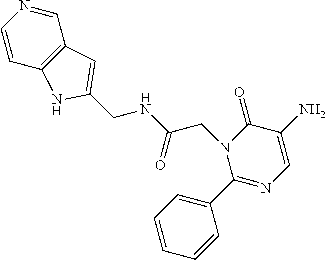 [SMILES: Nc1cnc(-c2ccccc2)n(CC(=O)NCc2cc3cnccc3[nH]2)c1=O] | ¹H NMR (400 MHz, CD₃OD) δ 7.51-7.34 (m, 13H), 7.31 (d, J = 7.2 Hz, 1H), 6.72 (s, 1H), 4.69 (s, 2H), 4.63 (s, 2H), 4.60 (s, 2H). LC/MS (ESI) m/z: 508 (M + H)⁺. RT (Method A): 0.95 min. |

### Scheme 27. Synthesis of N-((1H-pyrrolo[3,2-c]pyridine-2-yl)methyl)-2-(5-phenyl-2-((3-phenylpropyl)amino)91yridine-4-yl)acetamide (Compound 80)

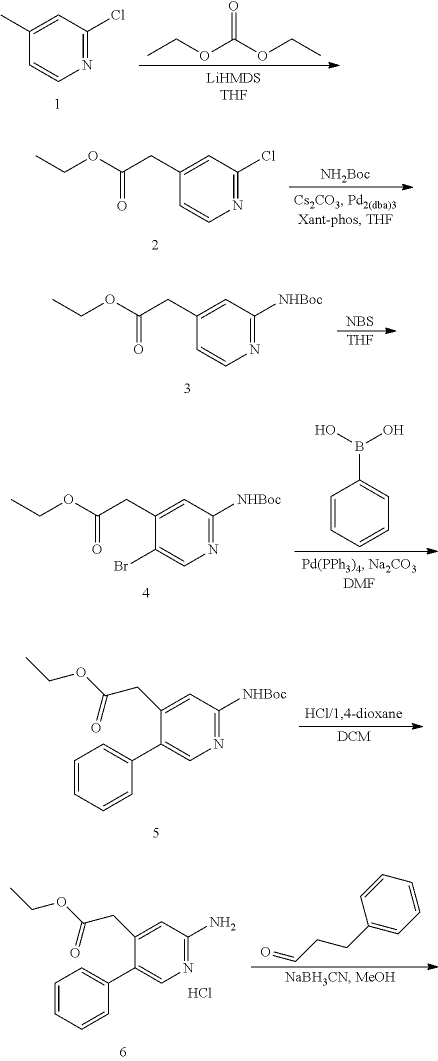

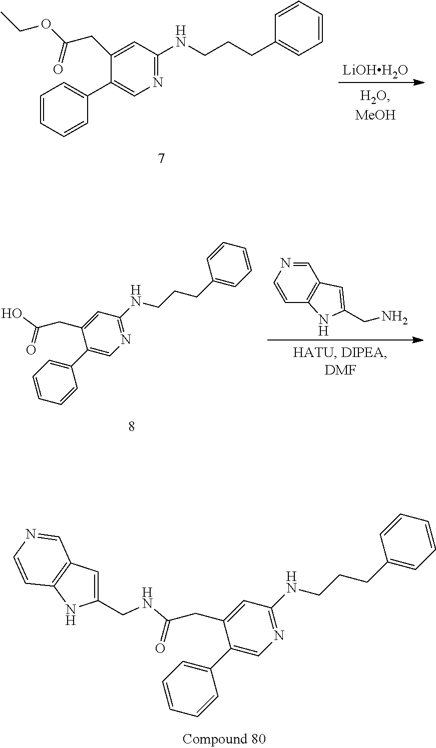

### Step 1: Ethyl 2-(2-chloropyridin-4-yl)acetate (2)

To a solution of 2-chloro-4-methylpyridine (5.0 g, 39.4 mmol) in THF (50 mL) was added LiHMDS (48 mL, 47.3 mmol, 1 mol/L in THF) drop-wisely at −78° C. and the mixture was stirred at −78° C. for 1 hour. Diethyl carbonate (7.0 g, 59.1 mmol) was added to the mixture at −78° C. and the resulting mixture was stirred at −78° C. for 2 hours. The mixture was quenched with saturated aq. NH₄Cl solution and extracted with EtOAc twice. The combined organic layers were washed with brine, dried over anhydrous Na₂SO₄, filtered, and concentrated to dryness. The residue was purified by flash chromatography o (silica gel, 0-20% EtOAc in PE) to give the title compound (3.5 g, yield 44.9%) as a yellow oil. LC/MS (ESI) m/z: 200 (M+H)⁺.

### Step 2: Ethyl 2-(2-((tert-butoxycarbonyl)amino)91yridine-4-yl)acetate (3)

To a mixture of ethyl 2-(2-chloropyridin-4-yl)acetate (1.5 g, 7.5 mmol) and NH₂Boc (2.6 g, 22.6 mmol) in THF (20 mL) was added Cs₂CO₃ (3.68 g, 11.3 mmol), Xant-Phos (218 mg, 0.4 mmol) and Pd₂(dba)₃ (173 mg, 0.2 mmol) under N₂ atmosphere, the mixture was degassed under N₂ atmosphere for ten times and stirred under N₂ atmosphere at 75° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-20% EtOAc in PE) to give the title compound (2.1 g, yield 99.5%) as a white solid. LC/MS (ESI) m/z: 281 (M+H)⁺.

### Step 3: Ethyl 2-(5-bromo-2-((tert-butoxycarbonyl)amino)91yridine-4-yl)acetate (4)

To a solution of ethyl 2-(2-((tert-butoxycarbonyl)amino)91yridine-4-yl)acetate (1.6 g, 5.7 mmol) in THF (20 mL) was added NBS (1.2 g, 6.8 mmol) at 0° C. and the reaction mixture was stirred at room temperature for 2 hours. The mixture was diluted with EtOAc, washed with brine, dried over anhydrous Na₂SO₄, filtered, and concentrated under reduced pressure to dryness. The residue was purified by flash chromatography (silica gel, 0-20% EtOAc in PE) to give the title compound (725 mg, yield 36.3%) as a white solid. LC/MS (ESI) m/z: 359/361 (M+H)⁺.

### Step 4: Ethyl 2-(2-((tert-butoxycarbonyl)amino)-5-phenylpyridin-4-yl)acetate (5)

To a mixture of ethyl 2-(5-bromo-2-((tert-butoxycarbonyl)amino)92yridine-4-yl)acetate (750 mg, 2.1 mmol) and phenylboronic acid (383 mg, 3.2 mmol) in 1,4-dioxane (9 mL) and H₂O (1.5 mL) was added Na₂CO₃ (666 mg, 9.6 mmol) and Pd(PPh₃)₄ (242 mg, 0.21 mmol) under N₂ atmosphere, the mixture was degassed under N₂ atmosphere for ten times and stirred under N₂ atmosphere at 80° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-20% EtOAc in PE) to give the title compound (740 mg, yield 99.2%) as a white solid. LC/MS (ESI) m/z: 357 (M+H)⁺.

### Step 5: Ethyl 2-(2-amino-5-phenylpyridin-4-yl)acetate hydrochloride (6)

To a solution of ethyl 2-(2-((tert-butoxycarbonyl)amino)-5-phenylpyridin-4-yl)acetate (740 mg, 2.1 mmol) in DCM (1 mL) was added HCl/1,4-dioxane (9 mL) and the reaction mixture was stirred at room temperature for 2 hours. The mixture was concentrated under reduced pressure to dryness to give the title compound (550 mg, crude) as a white solid, which was used directly in the next step without further purification. LC/MS (ESI) m/z: 257 (M+H)⁺.

### Step 6: Ethyl 2-(5-phenyl-2-((3-phenylpropyl)amino)pyridine-4-yl)acetate (7)

To a mixture of ethyl 2-(2-amino-5-phenylpyridin-4-yl)acetate hydrochloride (114 mg, 0.39 mmol) and 3-phenylpropanal (209 mg, 1.56 mmol) in MeOH (5 mL) was added NaBH₃CN (196 mg, 2.34 mmol) under N₂ atmosphere and the reaction mixture was stirred at 50° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-80% EtOAc in PE) to give the title compound (120 mg, yield 82.2%) as a colorless oil. LC/MS (ESI) m/z: 375 (M+H)⁺.

### Step 7: 2-(5-phenyl-2-((3-phenylpropyl)amino)pyridine-4-yl)acetic acid (8)

To a solution of ethyl 2-(5-phenyl-2-((3-phenylpropyl)amino)pyridine-4-yl)acetate (120 mg, 0.32 mmol) in MeOH (3 mL) and water (1 mL) was added LiOH·H₂O (27 mg, 0.64 mmol) and the reaction mixture was stirred at 25° C. for 2 hours. The mixture was acidified with 1 N aq. HCl to pH˜6 and extracted with EtOAc twice. The combined organic layers were dried over anhydrous Na₂SO₄, filtered, and concentrated under reduced pressure to dryness to give the title compound (100 mg, yield 90.1%) as a white solid, which was used directly in the next step without further purification. LC/MS (ESI) m/z: 347 (M+H)⁺.

### Step 8: N-((1H-pyrrolo[3,2-c]pyridine-2-yl)methyl)-2-(5-phenyl-2-((3-phenylpropyl) amino)pyridine-4-yl)acetamide (Compound 80)

To a mixture of 2-(5-phenyl-2-((3-phenylpropyl)amino)pyridine-4-yl)acetic acid (80 mg, 0.23 mmol) and (1H-pyrrolo[3,2-c]pyridine-2-yl)methanamine hydrochloride (43 mg, 0.46 mmol) in DMF (3 mL) was added DIPEA (179 mg, 1.38 mmol) and HATU (131 mg, 0.35 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-HPLC to give Compound 80 (5 mg, yield 4.5%) as a white solid. ¹H NMR (400 MHz, CD₃OD) δ 8.69 (s, 1H), 8.10 (d, J=5.8 Hz, 1H), 7.76 (s, 1H), 7.36 (d, J=5.8 Hz, 1H), 7.27-7.23 (m, 7H), 7.21-7.15 (m, 3H), 6.48 (s, 1H), 6.42 (s, 1H), 4.45 (s, 2H), 3.49 (s, 2H), 3.26 (d, J=7.0 Hz, 2H), 2.72-2.68 (m, 2H), 1.93-1.89 (m, 2H). LC/MS (ESI) m/z: 476 (M+H)⁺. RT (Method A): 0.86 min.

### Scheme 28. Synthesis of 2-(5-(1H-benzo[d]pyridine-2-yl)-6-oxo-2-phenylpyrimidin-1(6H)-yl)-N-((1H-pyrrolo[3,2-c]pyridine-2-yl)methyl)acetamide (Compound 82)

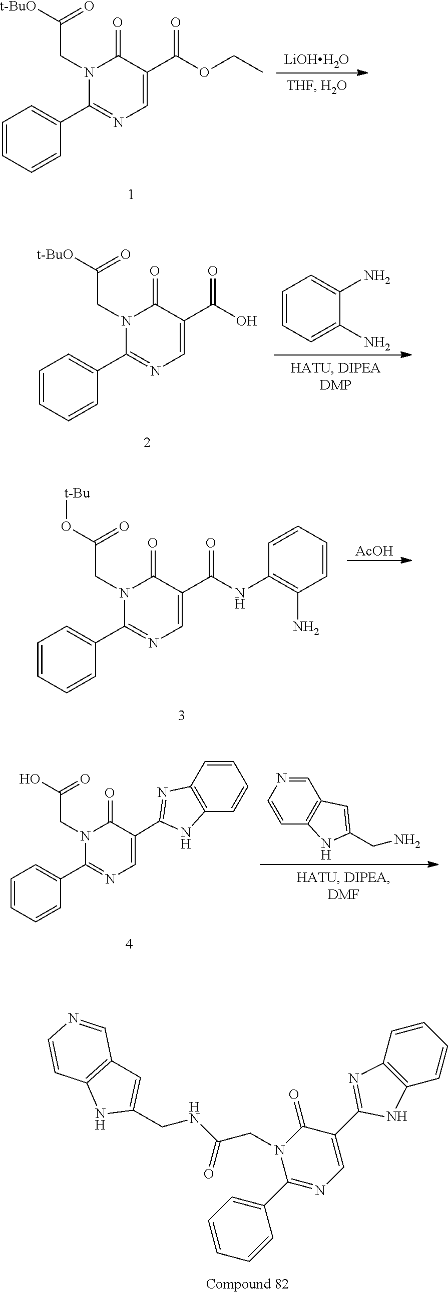

### Step 1: 1-(2-(tert-butoxy)-2-oxoethyl)-6-oxo-2-phenyl-1,6-dihydropyrimidine-5-carboxylic acid (2)

To a solution of ethyl 1-(2-(tert-butoxy)-2-oxoethyl)-6-oxo-2-phenyl-1,6-dihydropyrimidine-5-carboxylate (200 mg, 0.56 mmol) in THF (3 mL) and water (1 mL) was added LiOH·H₂O (23 mg, 0.56 mmol) and the reaction mixture was stirred at −5° C. for 2 hours. The mixture was acidified with 1 N aq. HCl to pH˜3 and extracted with EtOAc twice. The combined organic layers were dried over anhydrous Na₂SO₄, filtered, and concentrated under reduced pressure to dryness to give the title compound (150 mg, yield 81.5%) as a white solid, which was used directly in the next step without further purification. LC/MS (ESI) m/z: 331 (M+H)⁺.

### Step 2: Tert-butyl 2-(5-((2-aminophenyl)carbamoyl)-6-oxo-2-phenylpyrimidin-1(6H)-yl)acetate (3)

To a mixture of 1-(2-(tert-butoxy)-2-oxoethyl)-6-oxo-2-phenyl-1,6-dihydropyrimidine-5-carboxylic acid (190 mg, 0.58 mmol) and benzene-1,2-diamine (76 mg, 0.70 mmol) in DMF (5 mL) was added DIPEA (450 mg, 3.48 mmol) and HATU (332 mg, 0.87 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-25% EtOAc in PE) to give the title compound (100 mg, yield 40.8%) as a yellow solid. LC/MS (ESI) m/z: 421 (M+H)⁺.

### Step 3: 2-(5-(1H-benzo[d]pyridine-2-yl)-6-oxo-2-phenylpyrimidin-1(6H)-yl)acetic acid (4)

A solution of tert-butyl 2-(5-((2-aminophenyl)carbamoyl)-6-oxo-2-phenylpyrimidin-1(6H)-yl)acetate (80 mg, 0.20 mmol) in AcOH (2 mL) was stirred in a CEM microwave reactor at 120° C. for 2 hours. The mixture was filtered, and the filter cake was dried under vacuum to give the title compound (30 mg, yield 45.5%) as a white solid. LC/MS (ESI) m/z: 347 (M+H)⁺.

### Step 4: 2-(5-(1H-benzo[d]pyridine-2-yl)-6-oxo-2-phenylpyrimidin-1(6H)-yl)-N-((1H-pyrrolo[3,2-c]pyridine-2-yl)methyl)acetamide (Compound 82)

To a mixture of 2-(5-(1H-benzo[d]pyridine-2-yl)-6-oxo-2-phenylpyrimidin-1(6H)-yl)acetic acid (30 mg, 0.09 mmol) and (1H-pyrrolo[3,2-c]pyridine-2-yl)methanamine hydrochloride (25 mg, 0.18 mmol) in DMF (2 mL) was added DIPEA (70 mg, 0.54 mmol) and HATU (51 mg, 0.14 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-HPLC to give Compound 82 (3 mg, yield 7.3%) as a white solid. ¹H NMR (400 MHz, CD₃OD) δ 9.43 (s, 1H), 8.46 (s, 1H), 8.32 (d, J=7.3 Hz, 2H), 8.05 (d, J=6.2 Hz, 1H), 7.70-7.66 (m, 2H), 7.39 (d, J=7.3 Hz, 1H), 7.31 (dt, J=7.2, 4.3 Hz, 5H), 6.54 (s, 1H), 5.28 (s, 2H), 4.66 (s, 2H). LC/MS (ESI) m/z: 476 (M+H)⁺. RT (Method A): 1.00 min.

Compounds 81 and 425 were prepared based on Scheme 28:

| # | Reactant A | Reactant B | Reactant C | Characterization Data |
|---|---|---|---|---|
| 81ª | 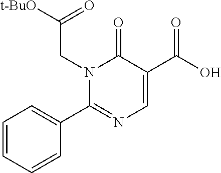 [SMILES: CC(C)(C)OC(=O)Cn1c(-c2ccccc2)ncc(C(=O)O)c1=O] | 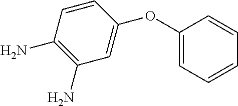 [SMILES: Nc1ccc(Oc2ccccc2)cc1N] | 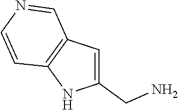 [SMILES: NCc1cc2cnccc2[nH]1] | ¹H NMR (400 MHz, CD₃OD) δ 9.41 (s, 1H), 8.39 (s, 1H), 8.36 (s, 1H), 8.34 (d, J = 1.3 Hz, 1H), 8.00 (d, J = 5.9 Hz, 1H), 7.65 (d, J = 8.0 Hz, 1H), 7.42 (t, J = 7.3 Hz, 1H), 7.33 (dd, J = 8.6, 7.3 Hz, 4H), 7.24 (s, 1H), 7.19 (d, J = 5.8 Hz, 1H), 7.10 (t, J = 7.4 Hz, 1H), 7.03 (dd, J = 13.9, 5.1 Hz, 3H), 6.43 (s, 1H), 5.28 (s, 2H), 4.63 (s, 2H). |
| | | | | LC/MS (ESI) m/z: 568 (M + |
| | | | | H)⁺. RT (Method A): 1.58 min. |

| | | | |
|---|---|---|---|
| 425^{b} | 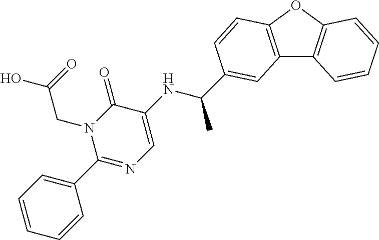 | 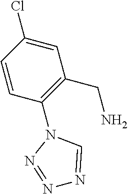 [SMILES: NCc1cc(Cl)ccc1-n1cnnn1] | ¹H NMR (400 MHz, CD₃OD) δ 9.55 (s, 1H), 8.09 (s, 1H), 8.03 (d, J = 7.8 Hz, 1H), 7.69 (s, 1H), 7.61-7.55 (m, 4H), 7.53-7.44 (m, 3H), 7.39 (t, J = 8.6 Hz, 5H), 6.90 (s, 1H), 4.69 (m, 1H), 4.54 (m, 2H), 4.23 (s, 2H), 1.68 (d, J = 6.4 Hz, 3H). LC/MS (ESI) m/z: 631 (M + H)⁺. RT (Method A): 2.66 min. |

^{a}Steps 2-4 only.
^{b}Step 4 only.

### Scheme 29. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-1,4(1,4)-dibenzenacyclohexaphane-12-carboxamide (Compound 83)

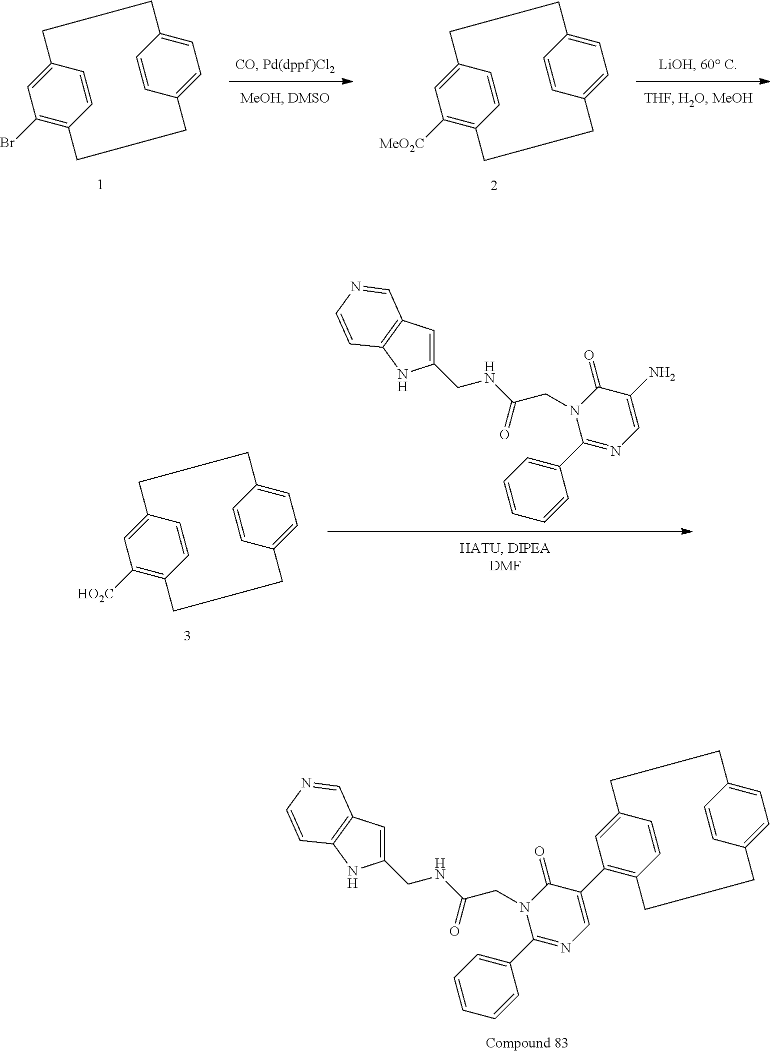 [SMILES: Brc1cc2ccc1CCc1ccc(cc1)CC2 ; COC(=O)c1cc2ccc1CCc1ccc(cc1)CC2 ; O=C(O)c1cc2ccc1CCc1ccc(cc1)CC2 ; Nc1cnc(-c2ccccc2)n(CC(=O)NCc2cc3cnccc3[nH]2)c1=O ; O=C(Cn1c(-c2ccccc2)ncc(-c2cc3ccc2CCc2ccc(cc2)CC3)c1=O)NCc1cc2cnccc2[nH]1]

### Step 1: Methyl 1,4(1,4)-dibenzenacyclohexaphane-1²-carboxylate (2)

To a mixture of 1²-bromo-1,4(1,4)-dibenzenacyclohexaphane (100 mg, 0.35 mmol) in 1,4-dioxane (15 mL) was added Pd(dppf)Cl₂ (25.4 mg, 0.035 mmol) under N₂ atmosphere at 0° C. The mixture was degassed under CO atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (57 mg, yield 61%) as an off-white solid. LC/MS (ESI) m/z: 267 (M+H)⁺.

### Step 2: 1,4(1,4)-Dibenzenacyclohexaphane-1²-carboxylic acid (3)

To a solution of methyl 1,4(1,4)-dibenzenacyclohexaphane-12-carboxylate (57 mg, 0.21 mmol) in THF/MeOH/H₂O (6 mL, 4/1/1) was added LiOH·H₂O (26.5 mg, 0.69 mmol) under N₂ atmosphere and the reaction mixture was stirred at 60° C. overnight. The mixture was acidified with 1N aq. HCl to pH˜3 and extracted with EtOAc twice. The combined organic layers were dried over anhydrous Na₂SO₄, filtered, and concentrated under reduced pressure to give the title compound (45 mg, yield 85%) as a white solid, which was used directly in the next step without further purification. LC/MS (ESI) m/z: 253 (M+H)⁺.

### Step 3: N-(1-(2-(((1H-pyrrolo[3,2-c]pyridine-2-yl)methyl)amino)-2-oxoethyl)-6-oxo-2-phenyl-1,6-dihydropyrimidin-5-yl)-1,4(1,4)-dibenzenacyclohexaphane-1²-carboxamide (Compound 83)

To a mixture of 1,4(1,4)-dibenzenacyclohexaphane-12-carboxylic acid (20 mg, 0.079 mmol) and N-((1H-pyrrolo[3,2-c]pyridine-2-yl)methyl)-2-(5-amino-6-oxo-2-phenylpyrimidin-1(6H)-yl)acetamide (35.4 mg, 0.095 mmol) in DMF (3 mL) was added DIPEA (51 mg, 0.395 mmol) and HATU (39 mg, 011 mol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=20:1) and further purified by prep-HPLC to give Compound 83 (3.2 mg, yield 6.7%) as a white solid. ¹H NMR (400 MHz, CD₃OD) δ 8.74 (s, 1H), 7.50 (t, J=6.1 Hz, 3H), 7.43 (t, J=7.3 Hz, 4H), 6.93-6.73 (m, 3H), 6.71 (d, J=5.1 Hz, 2H), 6.62 (s, 2H), 6.38 (d, J=8.0 Hz, 1H), 4.69 (s, 4H), 3.24-3.07 (m, 5H), 3.06-2.90 (m, 3H). LC/MS (ESI) m/z: 609 (M+H)⁺. RT (Method A): 1.58 min.

### Scheme 30. Synthesis of N-((1H-pyrrolo[3,2-c]pyridine-2-yl)methyl)-2-(5-((3-(4-(N-cyano-S-methylsulfonimidoyl)phenyl)propyl)amino)-6-oxo-2-phenylpyrimidin-1(6H)-yl)acetamide (Compound 84)

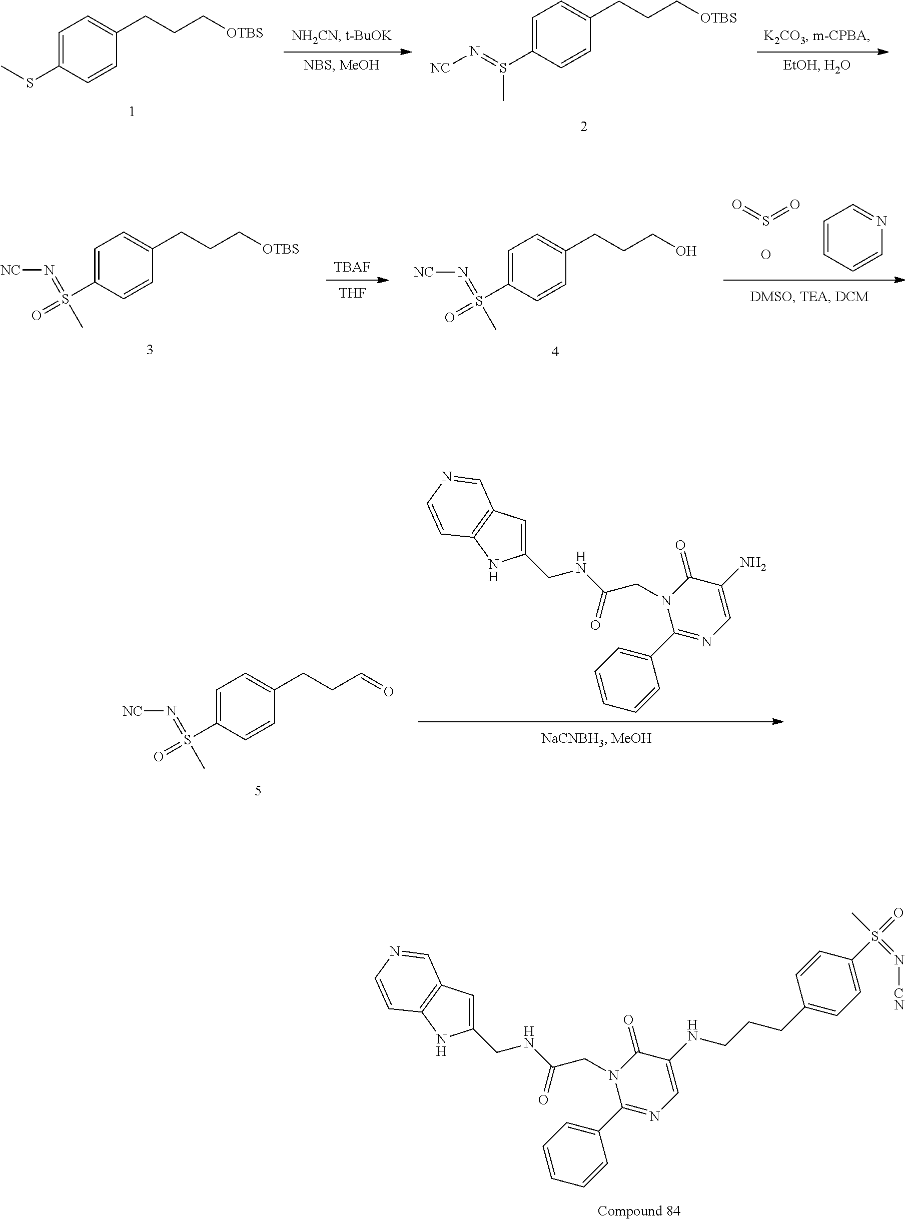

### Step 1: (E)-N-((4-(3-((tert-butyldimethylsilyl)oxy)propyl)phenyl)(methyl)-14-sulfanylidene)cyanamide (2)

To a solution of tert-butyldimethyl(3-(4-(methylthio)phenyl)propoxy)silane (150 mg, 0.51 mol) in MeCN (2 mL) was added NH₂CN (28 mg, 0.67 mmol), t-BuOK (0.6 mL, 1.18 mmol) and NBS (135 mg, 0.76 mmol) under N₂ atmosphere and the mixture was stirred at room temperature for 3 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (170 mg, yield 99.4%) as a colorless oil. LC/MS (ESI) (m/z): 337 (M+H)⁺.

### Step 2: N-((4-(3-((tert-butyldimethylsilyl)oxy)propyl)phenyl)(methyl)(oxo)-l6-sulfanylidene)cyanamide (2)

To a solution of (E)-N-((4-(3-((tert-butyldimethylsilyl)oxy)propyl)phenyl)(methyl)-4-sulfanylidene)cyanamide (120 mg, 0.36 mmol) in EtOH (0.9 mL) and H₂O (0.3 mL) was added K₂CO₃ (148 mg, 1.07 mmol) and m-CPBA (92 mg, 0.53 mmol) under N₂ atmosphere and the reaction mixture was stirred at room temperature overnight. The mixture was diluted with EtOAc, washed with aq. Na₂S₂O₃ solution and brine, dried over anhydrous Na₂SO₄, filtered, and concentrated under reduced pressure to dryness. The residue was purified by flash chromatography (silica gel, 0-50% EtOAc in PE) to give the title compound (85 mg, yield 67.6%) as a colorless oil. LC/MS (ESI) m/z: 353(M+H)⁺.

### Step 3: N-((4-(3-hydroxypropyl)phenyl)(methyl)(oxo)-l6-sulfanylidene)cyanamide (3)

To a solution of N-((4-(3-((tert-butyldimethylsilyl)oxy)propyl)phenyl)(methyl)(oxo)-l6-sulfanylidene)cyanamide (80 mg, 0.23 mmol) in THF (1 mL) was added TBAF (0.7 mL, 0.68 mmol, 1 M) at 0° C. and the mixture was stirred at room temperature for 1 hour. The mixture was diluted with EtOAc, washed with aq. NH₄Cl solution and brine, dried over anhydrous Na₂SO₄, filtered, and concentrated under reduced pressure to dryness. The residue was purified by flash chromatography (silica gel, 0-100% EtOAc in PE) to give the title compound (35 mg, yield 64.7%) as a colorless oil. LC/MS (ESI) m/z: 239 (M+H)⁺.

### Step 4: N-(methyl(oxo)(4-(3-oxopropyl)phenyl)-l6-sulfanylidene)cyanamide (4)

To a solution of N-((4-(3-hydroxypropyl)phenyl)(methyl)(oxo)-l6-sulfanylidene) cyanamide (35 mg, 0.15 mmol) in DCM (1 mL) was added DMSO (328 mg, 4.19 mmol), Py·SO₃ (100 mg, 0.62 mmol) and TEA (98 mg, 0.97 mmol) at 0° C. and the mixture was stirred under N₂ atmosphere at room temperature for 2 hours. The mixture was diluted with water and extracted with DCM twice. The combined organic layers were washed with aq. Na₂S₂O₃ solution, dried over anhydrous Na₂SO₄, filtered, and concentrated under reduced pressure to give the title compound (20 mg, yield 57.1%) as a colorless oil, which was used directly in the next step without further purification. LC/MS (ESI) m/z: 237 (M+H)⁺.

### Step 5: N-((1H-pyrrolo[3,2-c]pyridine-2-yl)methyl)-2-(5-((3-(4-(N-cyano-S-methylsulfonimidoyl)phenyl)propyl)amino)-6-oxo-2-phenylpyrimidin-1(6H)-yl)acetamide (Compound 84)

To a mixture of N-((1H-pyrrolo[3,2-c]pyridine-2-yl)methyl)-2-(5-amino-6-oxo-2-phenylpyrimidin-1(6H)-yl)acetamide (25 mg, 0.067 mol) and N-(methyl(oxo)(4-(3-oxopropyl)phenyl)-l6-sulfanylidene)cyanamide (20 mg, 0.084 mmol) in MeOH (1 mL) was added NaBH₃CN (12 mg, 0.19 mol) at 0° C. and the reaction mixture was stirred at 50° C. for 4 hours. The mixture was concentrated under reduced pressure to dryness. The residue was purified by prep-HPLC to give Compound 84 (1.1 mg, yield 2.8%) as a white solid. ¹H NMR (400 MHz, CD₃OD) δ 8.77 (s, 1H), 8.15 (d, J=6.0 Hz, 1H), 7.98 (d, J=8.5 Hz, 2H), 7.63 (d, J=8.4 Hz, 2H), 7.50-7.45 (m, 4H), 7.42-7.38 (m, 2H), 7.12 (s, 1H), 6.56 (s, 1H), 4.62 (s, 2H), 4.56 (s, 2H), 3.51 (s, 3H), 3.23 (d, J=6.8 Hz, 2H), 2.96-2.90 (m, 2H), 2.10-2.02 (m, 2H). LC/MS (ESI) m/z: 595 (M+H)⁺. RT (Method A): 1.02 min.

### Scheme 31. Synthesis of N-((1H-pyrrolo[3,2-c]pyridine-2-yl)methyl)-2-(3-methyl-4-((3-phenylpropyl)amino)-[1,1′-biphenyl]-2-yl)acetamide (Compound 85)

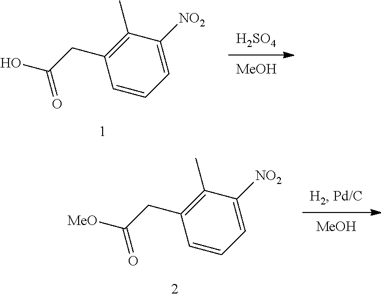

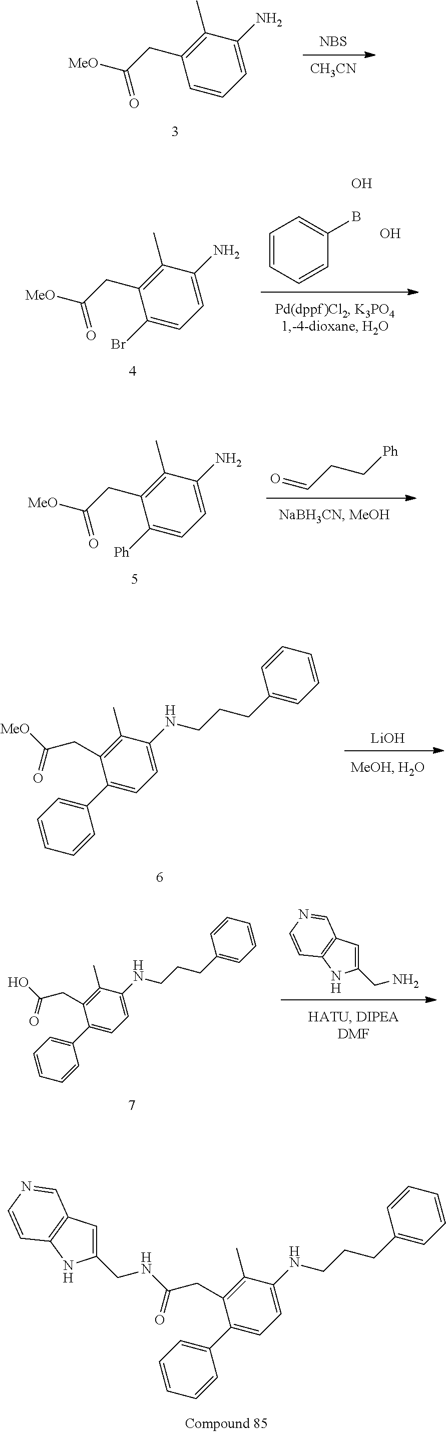

### Step 1: Methyl 2-(2-methyl-3-nitrophenyl)acetate (2)

To a mixture of 2-(2-methyl-3-nitrophenyl)acetic acid (1.95 g, 10 mmol) in MeOH (20 mL) was added sulfuric acid (0.18 g, 0.18 mmol) at room temperature and refluxed for 18 hours. The reaction mixture was cooled down to room temperature and added to 20% sodium carbonate solution (100 mL). The reaction mass was extracted with dichloromethane (50 mL×2). The combined organic layer was washed with water (100 mL), dried over sodium sulfate, and concentrated under reduced pressure to obtain the title product (1.9 g, 91.0%) as a colorless oil, which was used directly in the next step without further purification.

### Step 2: Methyl 2-(3-amino-2-methylphenyl)acetate (2)

To a solution of methyl 2-(2-methyl-3-nitrophenyl)acetate (1.9 g, 9 mmol) in MeOH (5 mL) was added Pd/C (20 mg, 10% wt.), the mixture was degassed under N₂ atmosphere for three times and stirred under a H₂ balloon at 25° C. overnight. The mixture was filtered, and the filtrate was concentrated to dryness to give title compound (0.78 g, yield 49%) as a yellow solid, which was used directly in the next step without further purification. LC/MS (ESI) m/z: 180 (M+H)⁺.

### Step 3: Methyl 2-(3-amino-6-bromo-2-methylphenyl)acetate (3)

To a solution of methyl 2-(3-amino-2-methylphenyl)acetate (0.78 g, 4.4 mmol) in CH₃CN (3 mL) was added N-bromosuccinimide (0.78 g, 4.4 mmol) at room temperature. After 2 hours, the reaction mixture was concentrated, and the residue partitioned between dichloromethane and water. The organic layer was washed with aq. Na₂S₂O₃ solution, dried over anhydrous Na₂SO₄, filtered, and concentrated under reduced pressure to dryness. The residue was purified by flash chromatography (silica gel, 0-25% EtOAc in PE) to give title compound (0.86 g, yield 76%) as a white solid. LC/MS (ESI) m/z: 258 (M+H)⁺.

### Step 4: Methyl 2-(4-amino-3-methyl-[1,1′-biphenyl]-2-yl)acetate (4)

To a mixture of methyl 2-(3-amino-6-bromo-2-methylphenyl) acetate (510 mg, 1.98 mmol) and phenylboronic acid (241.6 mg, 1.98 mmol) in water (3 mL) was added K₃PO₄ (1.3 g, 5.94 mmol) and Pd(PPh₃)₄ (110 mg, 0.096 mmol) at 25° C. under N₂ atmosphere and the reaction mixture was degassed under N₂ atmosphere for three times and stirred at 100° C. After 1 hours, the mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (460 mg, yield 91%) as a colorless solid. LC/MS (ESI) m/z: 256 (M+H)⁺.

### Step 5: Methyl 2-(3-methyl-4-((3-phenylpropyl)amino)-[1,1′-biphenyl]-2-yl)acetate (5)

To a mixture of methyl 2-(4-amino-3-methyl-[1,1′-biphenyl]-2-yl)acetate (60 mg, 0.23 mmol) and 3-phenylpropanal (94 mg, 0.70 mmol) in MeOH (2 mL) was added NaBH₃CN (86 mg, 0.70 mmol) at room temperature and the reaction mixture was stirred at 50° C. for 2 hours. The mixture was concentrated under reduced pressure to dryness. The residue was purified by prep-HPLC to give the title compound (23 mg, yield 26.8%) as a yellow oil. LC/MS (ESI) m/z: 374 (M+H)⁺.

### Step 6: 2-(3-Methyl-4-((3-phenylpropyl)amino)-[1,1′-biphenyl]-2-yl)acetic acid (6)

To a solution of methyl 2-(3-methyl-4-((3-phenylpropyl)amino)-[1,1′-biphenyl]-2-yl)acetate (28 mg, 0.075 mmol) in THF/MeOH/H₂O (6 mL, 4/1/1) was added LiOH·H₂O (9.5 mg, 0.225 mmol) under N₂ atmosphere and the reaction mixture was stirred at 100° C. overnight. The mixture was acidified with 1 N aq. HCl to pH˜3 and extracted with EtOAc twice. The combined organic layers were dried over anhydrous Na₂SO₄, filtered, and concentrated under reduced pressure to give the title compound (20 mg, yield 74.0%) as a white solid, which was used directly in the next step without further purification. LC/MS (ESI) m/z: 360(M+H)⁺.

### Step 7: N-((1H-pyrrolo[3,2-c]pyridine-2-yl)methyl)-2-(3-methyl-4-((3-phenylpropyl) amino)-[1,1′-biphenyl]-2-yl)acetamide (Compound 85)

To a mixture of 2-(3-methyl-4-((3-phenylpropyl)amino)-[1,1′-biphenyl]-2-yl)acetic acid (20 mg, 0.056 mmol) and (1H-pyrrolo[3,2-c]pyridine-2-yl)methanamine (24.7 mg, 0.168 mmol) in DMF (3 mL) was added DIPEA (36 mg, 0.28 mmol) and HATU (25.6 mg, 0.068 mol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=20:1) to give Compound 85 (2.4 mg, yield 8.9%) as a white solid. ¹H NMR (400 MHz, DMSO) δ 11.31 (s, 1H), 8.74 (s, 1H), 8.29 (t, J=5.7 Hz, 1H), 8.12 (d, J=5.6 Hz, 1H), 7.35-7.24 (m, 10H), 7.19 (t, J=7.0 Hz, 1H), 6.88 (d, J=8.3 Hz, 1H), 6.50 (d, J=8.4 Hz, 1H), 6.36 (s, 1H), 4.86 (t, J=5.5 Hz, 1H), 4.44 (d, J=5.6 Hz, 2H), 3.45 (s, 2H), 3.15-3.08 (m, 2H), 2.71 (t, J=7.6 Hz, 2H), 1.98 (s, 3H), 1.96-1.89 (m, 2H). LC/MS (ESI) m/z: 489 (M+H)⁺. RT (Method A): 1.92 min.

### Scheme 32. Synthesis of N-((1H-pyrrolo[3,2-c]pyridine-2-yl)methyl)-2-(2-(4-(oxetan-3-yloxy)phenyl)-6-oxo-5-((3-phenylpropyl)amino)pyrimidin-1(6H)-yl)acetamide (Compound 86)

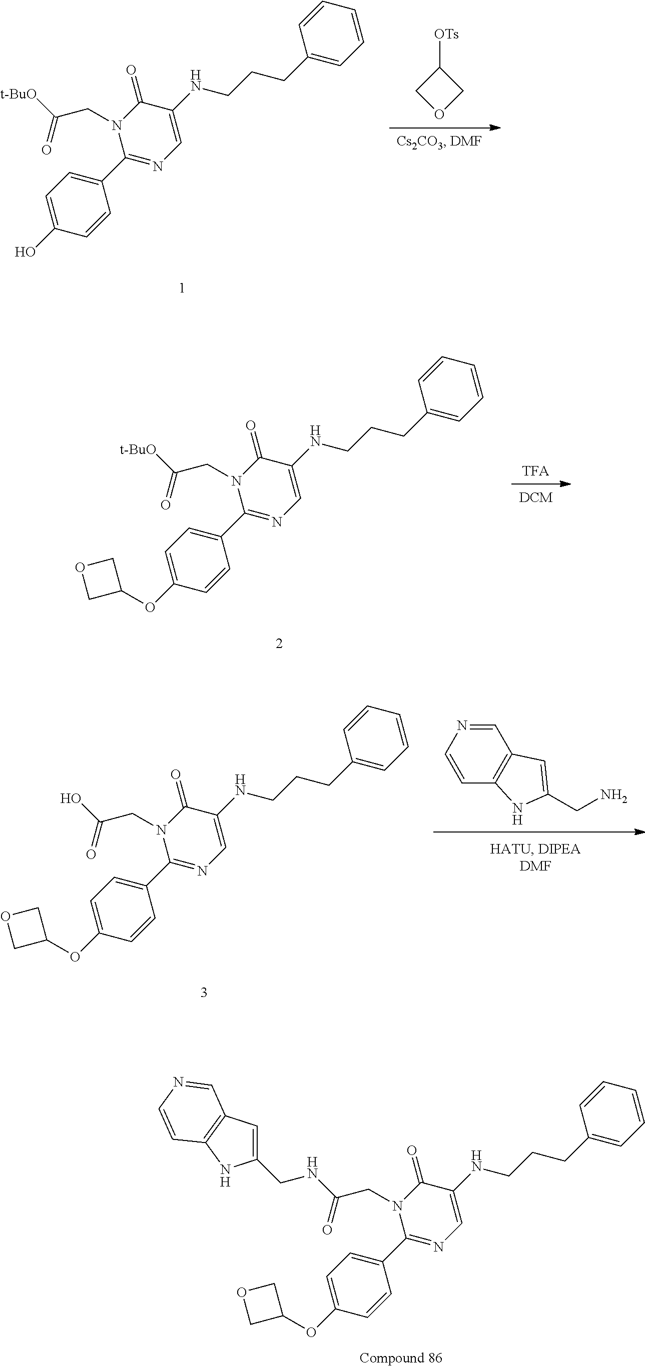

### Step 1: Tert-butyl 2-(2-(4-(oxetan-3-yloxy)phenyl)-6-oxo-5-((3-phenylpropyl)amino) pyrimidin-1(6H)-yl)acetate (2)

To a solution of tert-butyl 2-(2-(4-hydroxyphenyl)-6-oxo-5-((3-phenylpropyl) amino)pyrimidin-1(6H)-yl)acetate (96 mg, 0.22 mmol) and Cs₂CO₃ (0.66 mmol, 215 mg) in DMF (1 mL) was added oxetan-3-yl 4-methylbenzenesulfonate (50.2 mg, 0.22 mmol), and the reaction mixture was stirred at 100° C. for 10 hours. The residue was purified by flash chromatography (silica gel, 0-25% EtOAc in PE) to give the title compound (86 mg, yield 79%) as a pale-yellow solid. LC/MS (ESI) m/z: 492 (M+H)⁺.

### Step 2: 2-(2-(4-(Oxetan-3-yloxy)phenyl)-6-oxo-5-((3-phenylpropyl)amino)pyrimidin-1(6H)-yl)acetic acid (3)

To a solution of tert-butyl 2-(2-(4-(oxetan-3-yloxy)phenyl)-6-oxo-5-((3-phenylpropyl)amino)pyrimidin-1(6H)-yl)acetate (52 mg, 0.11 mmol) in DCM (1 mL) was added TFA (10 mL) and the mixture was stirred at room temperature for 16 hours. The mixture was concentrated, diluted with EtOAc, washed saturated aq. NaHCO₃ solution, dried over anhydrous Na₂SO₄, filtered, and concentrated under reduced pressure to dryness to give the title compound (41 mg, yield 85.0%) as a brown solid. LC/MS (ESI) m/z: 436 (M+H)*, which was used directly in the next step without further purification.

### Step 3: N-((1H-pyrrolo[3,2-c]pyridine-2-yl)methyl)-2-(2-(4-(oxetan-3-yloxy)phenyl)-6-oxo-5-((3-phenylpropyl)amino)pyrimidin-1(6H)-yl)acetamide (Compound 86)

To a mixture of 2-(2-(4-(oxetan-3-yloxy)phenyl)-6-oxo-5-((3-phenylpropyl)amino) pyrimidin-1(6H)-yl)acetic acid (25 mg, 0.057 mmol) and (1H-pyrrolo[3,2-c]pyridine-2-yl)methanamine (25.1 mg, 0.171 mmol) in DMF (3 mL) was added DIPEA (36.8 mg, 0.285 mmol) and HATU (32 mg, 0.084 mol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=20:1) and further purified by prep-HPLC to give Compound 86 (5.9 mg, yield 18.4%) as a white solid. ¹H NMR (400 MHz, CD₃OD) δ 8.74 (s, 1H), 8.13 (d, J=5.9 Hz, 1H), 7.43-7.39 (m, 2H), 7.30-7.14 (m, 5H), 7.06 (s, 1H), 6.72 (d, J=8.7 Hz, 2H), 6.50 (s, 1H), 5.18 (t, J=5.4 Hz, 1H), 4.95 (t, J=6.6 Hz, 2H), 4.63 (t, J=6.0 Hz, 4H), 4.54 (s, 2H), 3.17 (t, J=6.9 Hz, 2H), 2.74 (t, J=7.6 Hz, 2H), 2.02-1.90 (m, 2H). LC/MS (ESI) m/z: 564 (M+H)⁺. RT (Method A): 1.37 min.

### Scheme 33. Synthesis of N-((1H-pyrrolo[3,2-c]pyridine-2-yl)methyl)-2-(2-(((3R,3aR,6R,6aR)-6-methoxyhexahydrofuro[3,2-b]furan-3-yl)oxy)-6-oxo-5-((3-phenylpropyl)amino) pyrimidin-1(6H)-yl)acetamide (Compound 87)

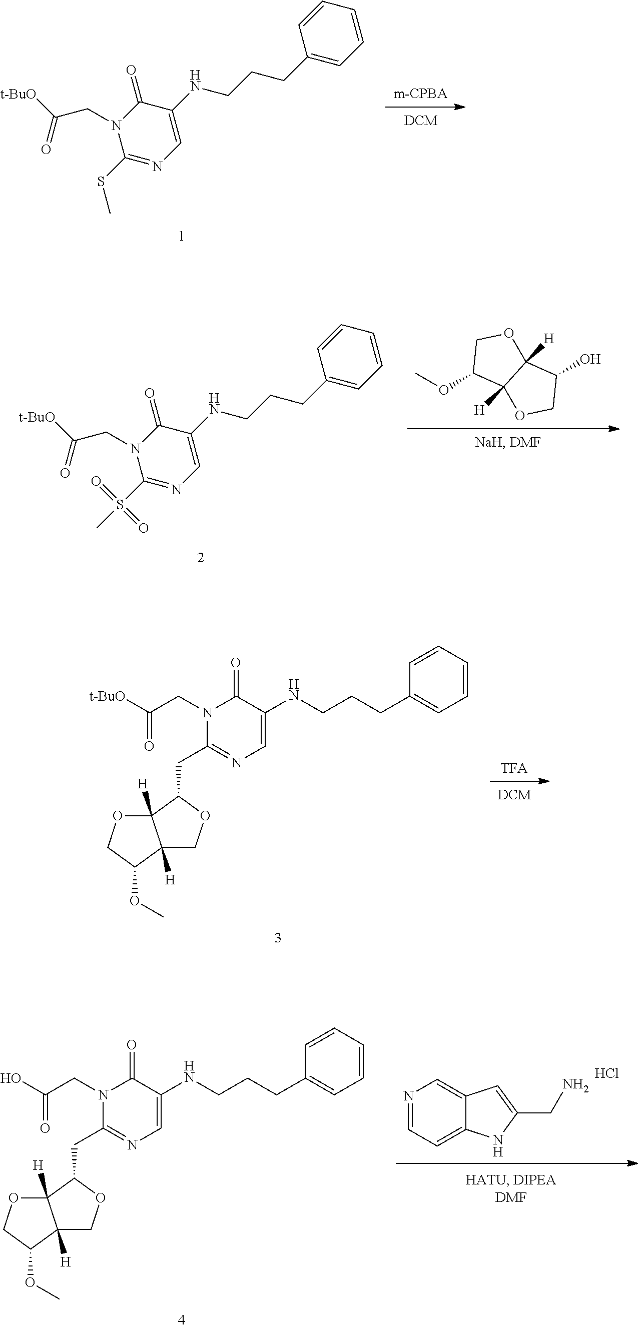

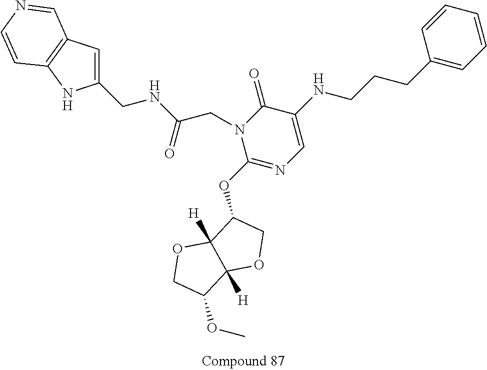 [SMILES: CO[C@@H]1CO[C@H]2[C@@H]1OC[C@H]2Oc1ncc(NCCCc2ccccc2)c(=O)n1CC(=O)NCc1cc2cnccc2[nH]1]

### Step 1: Tert-butyl 2-(2-(methylsulfonyl)-6-oxo-5-((3-phenylpropyl)amino)pyrimidin-1(6H)-yl)acetate (2)

To a solution of tert-butyl 2-(2-(methylsulfonyl)-6-oxo-5-((3-phenylpropyl)amino) pyrimidin-1(6H)-yl)acetate (50 mg, 0.13 mmol) in DCM (3 mL) was added m-CPBA (44 mg, 0.36 mmol) at 0° C. and the mixture was stirred at room temperature for 3 hours. The mixture was quenched with aq. Na₂S₂O₃ solution and extracted with DCM twice. The combined organic layers were washed with brine, dried over anhydrous Na₂SO₄, filtered, and concentrated under reduced pressure to dryness to give the title compound (54 mg, yield 99.8%) as a white solid, which was used directly in the next reaction without further purification. LC/MS (ESI) m/z: 422 (M+H)⁺.

### Step 2: tert-butyl 2-(2-(((3R,3aR,6R,6aR)-6-methoxyhexahydrofuro[3,2-b]furan-3-yl)oxy)-6-oxo-5-((3-phenylpropyl)amino)pyrimidin-1(6H)-yl)acetate (3)

To a solution of (3R,3aR,6R,6aR)-6-methoxyhexahydrofuro[3,2-b]furan-3-ol (20 mg, 0.12 mmol) in DMF (3 mL) was added NaH (8.0 mg, 0.20 mmol, 60% wt.) at 0° C. and the mixture was stirred at 0° C. for 0.5 hour. Then a solution of tert-butyl 2-(2-(methylsulfonyl)-6-oxo-5-((3-phenylpropyl)amino)pyrimidin-1(6H)-yl)acetate (50 mg, 0.12 mmol) in DMF (1 mL) was added and the resulting mixture was stirred at room temperature overnight. The mixture was quenched with saturated aq. NH₄Cl solution and extracted with EtOAc twice. The combined organic layers were washed with brine, dried over anhydrous Na₂SO₄, filtered, and concentrated to dryness. The residue was purified by prep-TLC (PE:EtOAc=1:1) to give the title compound (30 mg, yield 50.4%) as a light oil. LC/MS (ESI) m/z: 502 (M+H)⁺.

### Step 3: 2-(2-(((3R,3aR,6R,6aR)-6-methoxyhexahydrofuro[3,2-b]furan-3-yl)oxy)-6-oxo-5-((3-phenylpropyl)amino)pyrimidin-1(6H)-yl)acetic acid (4)

To a solution of tert-butyl 2-(2-(((3R,3aR,6R,6aR)-6-methoxyhexahydrofuro[3,2-b]furan-3-yl)oxy)-6-oxo-5-((3-phenylpropyl)amino)pyrimidin-1(6H)-yl)acetate (30 mg, 0.060 mmol) in DCM (2 mL) was added TFA (2 mL) and the reaction mixture was stirred at room temperature for 5 hours. The mixture was concentrated under reduced pressure to dryness to give the title compound (25 mg, yield 94.0%) as a yellow solid, which was used directly in the next step without further purification. LC/MS (ESI) m/z: 446 (M+H)⁺.

### Step 4: N-((1H-pyrrolo[3,2-c]pyridine-2-yl)methyl)-2-(2-(((3R,3aR,6R,6aR)-6-methoxyhexahydrofuro[3,2-b]furan-3-yl)oxy)-6-oxo-5-((3-phenylpropyl)amino) pyrimidin-1(6H)-yl)acetamide (Compound 87)

To a mixture of 2-(2-(((3R,3aR,6R,6aR)-6-methoxyhexahydrofuro[3,2-b]furan-3-yl)oxy)-6-oxo-5-((3-phenylpropyl)amino)pyrimidin-1(6H)-yl)acetic acid (25 mg, 0.056 mmol) and (1H-pyrrolo[3,2-c]pyridine-2-yl)methanamine hydrochloride (29 mg, 0.16 mmol) in DMF (2 mL) was added DIPEA (36 mg, 0.28 mmol) and HATU (32 mg, 0.084 mmol) under N₂ atmosphere and the reaction mixture was stirred at room temperature for 1 hour. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=10:1) and further purified by prep-HPLC to give Compound 87 (4.2 mg, yield 13.0%) as a white solid. ¹H NMR (400 MHz, CD₃OD) δ 8.71 (d, J=0.8 Hz, 1H), 8.10 (d, J=5.8 Hz, 1H), 7.38 (d, J=5.8 Hz, 1H), 7.29-7.23 (m, 2H), 7.22-7.13 (m, 3H), 6.80 (s, 1H), 6.56 (s, 1H), 5.31 (q, J=5.6 Hz, 1H), 4.89 (d, J=16.0 Hz, 1H), 4.79 (t, J=5.2 Hz, 1H), 4.73 (d, J=16.0 Hz, 1H), 4.57 (dd, J=9.2, 4.4 Hz, 3H), 3.99 (dd, J=9.9, 6.0 Hz, 1H), 3.96-3.91 (m, 1H), 3.91-3.86 (m, 1H), 3.83 (dd, J=9.9, 5.2 Hz, 1H), 3.51 (t, J=8.1 Hz, 1H), 3.42 (s, 3H), 3.04 (t, J=7.0 Hz, 2H), 2.74-2.69 (m, 2H), 1.95 (dd, J=15.0, 7.2 Hz, 2H). LC/MS (ESI) m/z: 575 (M+H)⁺. RT (Method A): 1.30 min.

### Scheme 34. Synthesis of diethyl ((4-(1-(2-(((1H-pyrrolo[3,2-c]pyridine-2-yl)methyl)amino)-2-oxoethyl)-6-oxo-5-((3-phenylpropyl)amino)-1,6-dihydropyrimidin-2-yl)phenoxy)methyl)phosphonate (Compound 89)

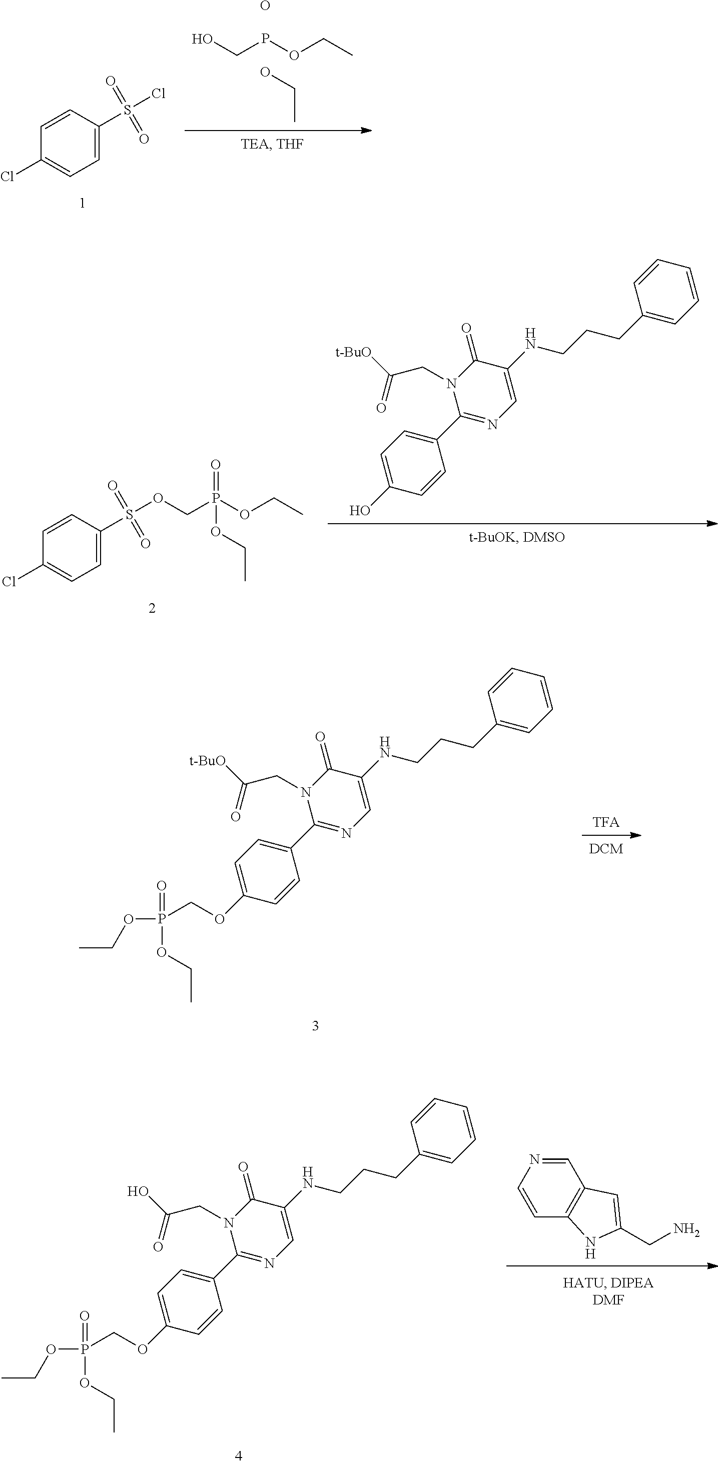

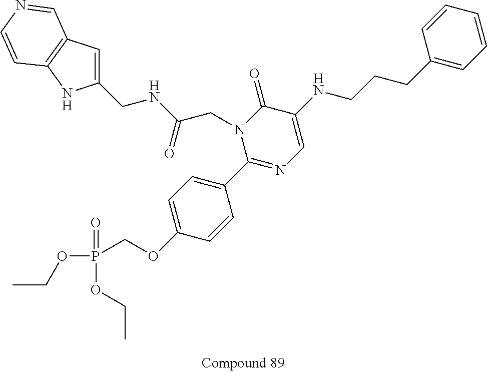 [SMILES: CCOP(=O)(COc1ccc(-c2ncc(NCCCc3ccccc3)c(=O)n2CC(=O)NCc2cc3cnccc3[nH]2)cc1)OCC]

### Step 1: (Diethoxyphosphoryl)methyl 4-chlorobenzenesulfonate (2)

To a mixture of diethyl (hydroxymethyl)phosphonate (2 g, 11.9 mmol) in THF (20 mL) was added TEA (1.82 mL, 13.0 mmol). Then 4-chlorobenzenesulfonyl chloride (2.7 g, 13.0 mmol) was added into the mixture at −10° C. The mixture was stirred under N₂ at 25° C. for 17 hours. The mixture was diluted with H₂O and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (3.2 g, yield 78%) as a yellow oil. LC/MS (ESI) (m/z): 343 (M+H)⁺.

### Step 2: Tert-butyl 2-(2-(4-((diethoxyphosphoryl)methoxy)phenyl)-6-oxo-5-((3-phenylpropyl)amino)pyrimidin-1(6H)-yl)acetate (3)

To a solution of tert-butyl 2-(2-(4-hydroxyphenyl)-6-oxo-5-((3-phenylpropyl)amino) pyrimidin-1(6H)-yl)acetate (100 mg, 0.229 mmol) in DMSO (2 mL) was added t-BuOK (52 mg, 0.45 mmol) and (diethoxyphosphoryl)methyl 4-chlorobenzenesulfonate (95 mg, 0.275 mmol). The mixture was stirred under N₂ at 25° C. for 3 hours. The mixture was diluted with H₂O and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (40 mg, yield 30%) as a white solid. LC/MS (ESI) (m/z): 586 (M+H)⁺.

### Step 3: 2-(2-(4-((Diethoxyphosphoryl)methoxy)phenyl)-5-(hex-5-en-1-ylamino)-6-oxopyrimidin-1(6H)-yl)acetic acid (4)

To a solution of tert-butyl 2-(2-(4-((diethoxyphosphoryl)methoxy)phenyl)-6-oxo-5-((3-phenylpropyl)amino)pyrimidin-1(6H)-yl)acetate (40 mg, 0.068 mmol) in HCl/dioxane (2 mL, 4 M). The mixture was stirred at 25° C. for 1 hour. The mixture was concentrated under reduced pressure to give the title compound (35 mg, yield 95%) as a white solid. LC/MS (ESI) (m/z): 494 (M+H)⁺.

### Step 4: Diethyl ((4-(1-(2-(((1H-pyrrolo[3,2-c]pyridine-2-yl)methyl)amino)-2-oxoethyl)-6-oxo-5-((3-phenylpropyl)amino)-1,6-dihydropyrimidin-2-yl)phenoxy)methyl) phosphonate (Compound 89)

To a solution of 2-(2-(4-((diethoxyphosphoryl)methoxy)phenyl)-5-(hex-5-en-1-ylamino)-6-oxopyrimidin-1(6H)-yl)acetic acid (35 mg, 0.065 mmol) in DMF (2 mL) was added HATU (75 mg, 0.098 mmol) and DIEA (0.04 mL, 0.195 mmol), then (1H-pyrrolo[3,2-c]pyridine-2-yl)methanamine (10 mg, 0.068 mmol) was added into the mixture. The mixture was stirred under N₂ at 25° C. for 0.5 hour. The mixture was diluted with EtOAc, dried over anhydrous Na₂SO₄, concentrated under reduced pressure to dryness. The residue was purified by prep-HPLC to give Compound 89 (4.6 mg, yield 10.7%) as a white solid. ¹H NMR (400 MHz, CD₃OD) δ 8.72 (d, J=0.9 Hz, 1H), 8.11 (d, J=5.8 Hz, 1H), 7.49-7.38 (m, 3H), 7.30-7.25 (m, 2H), 7.24-7.20 (m, 2H), 7.19-7.13 (m, 1H), 7.06 (s, 1H), 7.01-6.96 (m, 2H), 6.51 (d, J=0.6 Hz, 1H), 4.62 (s, 2H), 4.54 (s, 2H), 4.37 (d, J=10.0 Hz, 2H), 4.27-4.18 (m, 4H), 3.16 (t, J=7.0 Hz, 2H), 2.79-2.69 (m, 2H), 2.04-1.93 (m, 2H), 1.35 (t, J=7.1 Hz, 6H). LC/MS (ESI) (m/z): 659 (M+H)⁺. RT (Method A): 1.53 min.

### Scheme 35. Synthesis of (4-(3-((1-(2-(((1H-pyrrolo[3,2-c]pyridine-2-yl)methyl)amino)-2-oxoethyl)-6-oxo-2-phenyl-1,6-dihydropyrimidin-5-yl)amino)propyl)phenyl) (methyl)phosphinic acid (Compound 91)

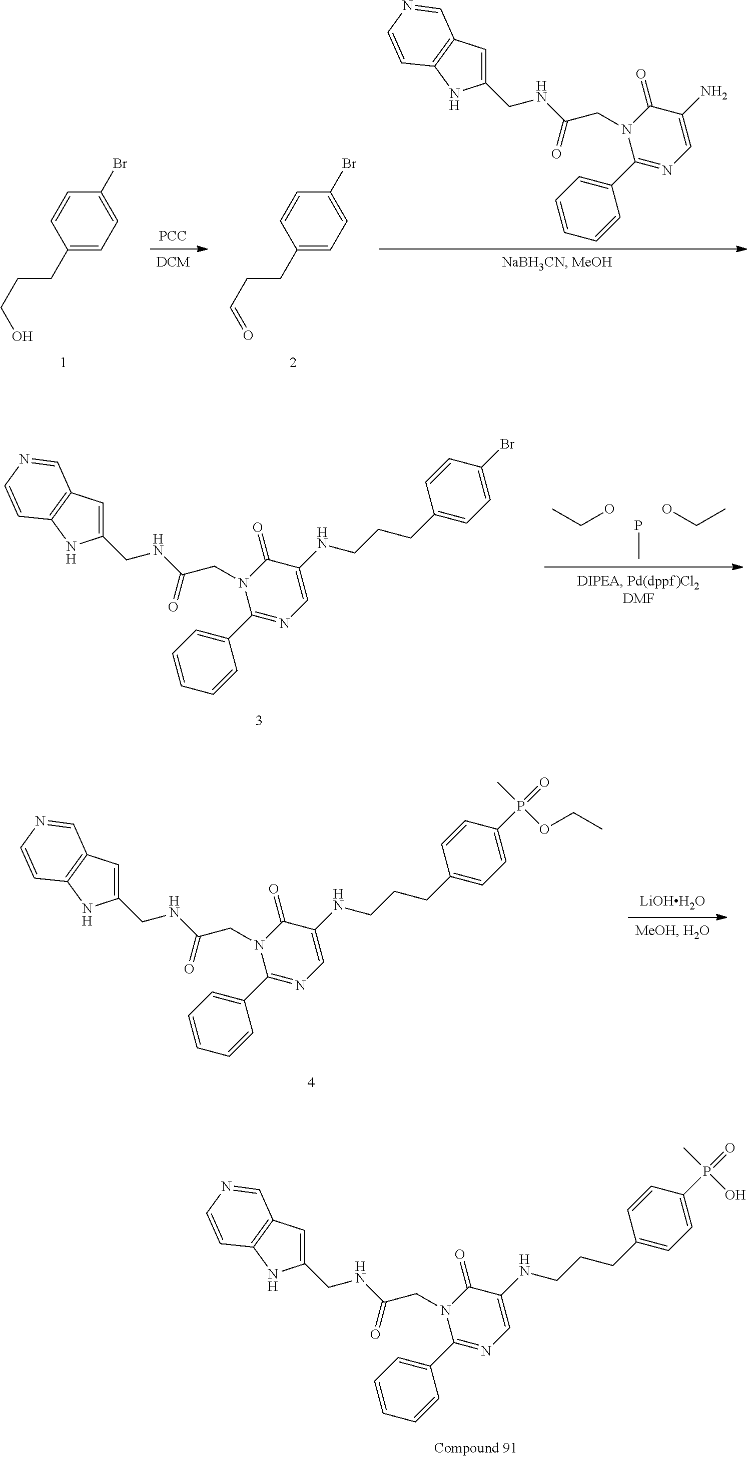

### Step 1: 3-(4-Bromophenyl)propanal (2)

To a solution of 3-(4-bromophenyl)propan-1-ol (1.0 g, 4.67 mmol) in DCM (10 mL) was added PCC (1.5 g, 6.96 mmol) at 0° C. and the mixture was stirred at room temperature for 1 hour. Silica gel was added, and the mixture was stirred at room temperature for 0.5 hour. The mixture was filtered, and the filtrate was concentrated to dryness to give the title compound (620 mg, yield 62.9%) as a colorless oil, which was used directly in the next reaction without further purification.

### Step 2: N-((1H-pyrrolo[3,2-c]pyridine-2-yl)methyl)-2-(5-((3-(4-bromophenyl) propyl) amino)-6-oxo-2-phenylpyrimidin-1(6H)-yl)acetamide (3)

To a mixture of N-((1H-pyrrolo[3,2-c]pyridine-2-yl)methyl)-2-(5-amino-6-oxo-2-phenylpyrimidin-1(6H)-yl)acetamide (100 mg, 0.27 mmol) and 3-(4-bromophenyl) propanal (171 mg, 0.81 mmol) in MeOH (3 mL) was added NaBH₃CN (134 mg, 2.1 mmol) under N₂ atmosphere and the reaction mixture was stirred at 50° C. overnight. The mixture was diluted with EtOAc, washed with brine, dried over anhydrous Na₂SO₄, filtered, and concentrated under reduced pressure to dryness. The residue was purified by flash chromatography (silica gel, 0-10% MeOH in DCM) to give the title compound (40 mg, yield 26.3%) as a white solid. LC/MS (ESI) m/z: 571 (M+H)⁺.

### Step 3: Ethyl (4-(3-((1-(2-(((1H-pyrrolo[3,2-c]pyridine-2-yl)methyl)amino)-2-oxoethyl)-6-oxo-2-phenyl-1,6-dihydropyrimidin-5-yl)amino)propyl)phenyl)(methyl) phosphinate (4)

To a mixture of N-((1H-pyrrolo[3,2-c]pyridine-2-yl)methyl)-2-(5-((3-(4-bromophenyl) propyl)amino)-6-oxo-2-phenylpyrimidin-1(6H)-yl)acetamide (40 mg, 0.07 mmol) and diethyl methylphosphonite (48 mg, 0.35 mmol) in DMF (2 mL) was added DIPEA (46 mg, 0.36 mmol) and Pd(dppf)Cl₂ (3 mg, 0.004 mmol) under N₂ atmosphere and the reaction mixture was stirred in a CEM Microwave Reactor at 130° C. for 30 minutes. The mixture was diluted with EtOAc, washed with water and brine, dried over anhydrous Na₂SO₄, filtered, and concentrated under reduced pressure to dryness. The residue was purified by prep-TLC (DCM:MeOH=10:1) to give the title compound (18 mg, yield 42.9%) as a white solid. LC/MS (ESI) m/z: 599 (M+H)⁺.

### Step 4: (4-(3-((1-(2-(((1H-pyrrolo[3,2-c]pyridine-2-yl)methyl)amino)-2-oxoethyl)-6-oxo-2-phenyl-1,6-dihydropyrimidin-5-yl)amino)propyl)phenyl)(methyl)phosphinic acid (Compound 91)

To a solution of ethyl (4-(3-((1-(2-(((1H-pyrrolo[3,2-c]pyridine-2-yl)methyl)amino)-2-oxoethyl)-6-oxo-2-phenyl-1,6-dihydropyrimidin-5-yl)amino)propyl)phenyl)(methyl) phosphinate (18 mg, 0.03 mmol) in MeOH (2 mL) and water (1 mL) was added LiOH·H₂O (4 mg, 0.09 mmol) and the reaction mixture was stirred at 25° C. overnight. The reaction mixture was concentrated under reduced pressure to dryness. The residue was purified by prep-HPLC to give Compound 91 (2.6 mg, yield 15.2%) as a white solid. ¹H NMR (400 MHz, CD₃OD) δ 8.95 (s, 1H), 8.36 (s, 1H), 8.23 (d, J=6.6 Hz, 1H), 7.83 (d, J=6.5 Hz, 1H), 7.70 (m, 2H), 7.53-7.47 (m, 3H), 7.44 (d, J=7.5 Hz, 2H), 7.24 (d, J=6.4 Hz, 2H), 7.14 (s, 1H), 6.86 (s, 1H), 4.63 (d, J=6.3 Hz, 4H), 3.19 (t, J=6.7 Hz, 2H), 2.75 (t, J=7.7 Hz, 2H), 2.02-1.96 (m, 2H), 1.40-1.36 (m, 3H). LC/MS (ESI) m/z: 571 (M+H)⁺. RT (Method A): 0.80 min.

### Scheme 36. Synthesis of N-((1H-pyrrolo[3,2-c]pyridine-2-yl)methyl)-2-(5-((3-(((3R,3aR,6R,6aR)-6-methoxyhexahydrofuro[3,2-b]furan-3-yl)oxy)propyl)amino)-6-oxo-2-phenylpyrimidin-1(6H)-yl)acetamide (Compound 5)

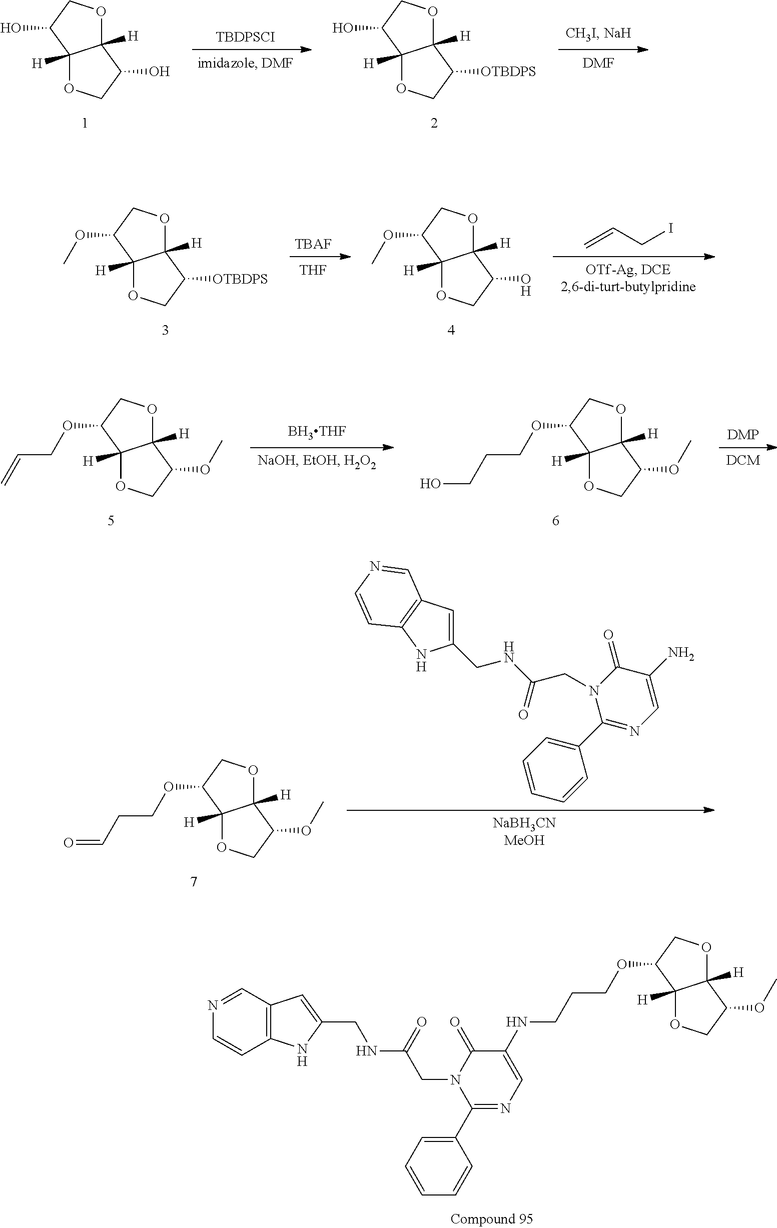

### Step 1: (3R,3aR,6R,6aS)-6-((tert-butyldiphenylsilyl)oxy)hexahydrofuro[3,2-b]furan-3-ol (2)

To a solution of (3R,3aR,6R,6aR)-hexahydrofuro[3,2-b]furan-3,6-diol (15.0 g, 102.7 mmol) in DMF (30 mL) was added TBDPSCl (33.9 g, 123.2 mmol) and imidazole (21.0 g, 308.2 mmol) at 0° C. and the mixture was stirred at room temperature for 4 hours. The mixture was diluted with water and extracted with EtOAc twice. The combined organic layers were washed with brine, dried over anhydrous Na₂SO₄, filtered, and concentrated to dryness. The residue was purified by flash chromatography (silica gel, 0-20% EtOAc in PE) to give the title compound (13.5 g, yield 34.2%) as a colorless oil. ¹H NMR (400 MHz, CDCl₃) δ 7.78-7.73 (m, 2H), 7.70-7.66 (m, 2H), 7.47-7.38 (m, 6H), 4.35 (d, J=5.2 Hz, 1H), 4.26-4.20 (m, 3H), 4.02 (m, 1H), 3.78-3.72 (m, 2H), 3.68-3.63 (m, 1H). LC/MS (ESI) m/z: 407(M+Na)⁺.

### Step 2: Tert-butyl(((3R,3aS,6R,6aR)-6-methoxyhexahydrofuro[3,2-b]furan-3-yl)oxy) diphenylsilane (3)

To a solution of (3R,3aR,6R,6aS)-6-((tert-butyldiphenylsilyl)oxy)hexahydrofuro[3,2-b]furan-3-ol (5.0 g, 13.0 mmol) in DMF (50 mL) was added NaH (469 mg, 19.5 mmol, 60 wt. %) at 0° C. and the reaction solution was stirred at 0° C. for 0.5 hours. The mixture was added Iodomethane (2.8 g, 19.5 mmol) at 0° C. and the resulting mixture was stirred at room temperature for 3 hours. The mixture was quenched with aq. NH₄Cl and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (2.3 g, yield 44.4%) as a colorless oil. LC/MS (ESI) m/z: 421 (M+Na)⁺.

### Step 3: (3R,3aR,6R,6aR)-6-methoxyhexahydrofuro[3,2-b]furan-3-ol (4)

To a solution of tert-butyl(((3R,3aS,6R,6aR)-6-methoxyhexahydrofuro[3,2-b]furan-3-yl)oxy)diphenylsilane (2.2 g, 5.53 mmol) in THF (25 mL) was added TBAF (1.9 mL, 3.9 mmol) and the reaction mixture was stirred at room temperature overnight. The mixture was diluted with aq. NH₄Cl and extracted with CHCl₃/i-PrOH (3/1, v/v) twice. The combined organic layers were washed with brine, dried over anhydrous Na₂SO₄, filtered, and concentrated to dryness. The residue was purified by flash chromatography (silica gel, 0-15% MeOH in DCM) to give the title compound (700 mg, yield 79.2%) as a white solid. ¹H NMR (400 MHz, CDCl₃) δ 4.57 (t, J=4.8 Hz, 1H), 4.51 (t, J=5.2 Hz, 1H), 4.27 (q, J=5.9 Hz, 1H), 4.07 (m, 1H), 4.01-3.92 (m, 2H), 3.73-3.65 (m, 2H), 3.47 (s, 3H).

### Step 4: (3R,3aR,6R,6aR)-3-(allyloxy)-6-methoxyhexahydrofuro[3,2-b]furan (5)

To a solution of (3R,3aR,6R,6aR)-6-methoxyhexahydrofuro[3,2-b]furan-3-ol (300 mg, 1.9 mmol) in DCE (10 mL) was added AgOTf (1.4 g, 5.6 mmol) followed by 3-iodoprop-1-ene (1.3 g, 7.5 mmol), 2,6-di-tert-butylpyridine (1.4 g, 7.5 mmol) and the mixture was stirred at 30° C. for 16 hours under N₂ atmosphere. The mixture was diluted with water and extracted with EtOAc twice. The combined organic layers were washed with brine, dried over anhydrous Na₂SO₄, filtered, and concentrated to dryness. The residue was purified by flash chromatography (silica gel, 0-15% MeOH in DCM) to give the title compound (320 mg, yield 85.3%) as a yellow oil. ¹H NMR (400 MHz, CDCl₃) δ 5.93 (m, 1H), 5.29 (m, 1H), 5.20 (m, 1H), 4.58-4.53 (m, 2H), 4.19-4.14 (m, 1H), 4.07-4.02 (m, 4H), 3.96-3.91 (m, 1H), 3.72-3.67 (m, 2H), 3.45 (s, 3H).

### Step 5: 3-(((3R,3aR,6R,6aR)-6-methoxyhexahydrofuro[3,2-b]furan-3-yl)oxy)propan-1-ol (6)

To a solution of (3R,3aR,6R,6aR)-3-(allyloxy)-6-methoxyhexahydrofuro[3,2-b]furan (160 mg, 0.8 mmol) in THF (2 mL) was added BH₃·THF (1.1 mL, 1.1 mmol) drop-wisely at 0° C. and the reaction was stirred at 0° C. for 2.5 hours. The mixture was added the solution of NaOH (1.0 mL, 10M in water) and H₂O₂ (1.0 mL, 30 wt. %) successively at 0° C. The resulting mixture was stirred at room temperature for 0.5 hour. The mixture was added water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (120 mg, yield 68.8%) as a colorless oil. ¹H NMR (400 MHz, CDCl₃) δ 4.62 (m, 2H), 4.06 (t, J=7.2 Hz, 3H), 3.92 (m, 2H), 3.78-3.65 (m, 5H), 3.46 (s, 3H), 1.83 (m, 2H).

### Step 6: 3-(((3R,3aR,6R,6aR)-6-methoxyhexahydrofuro[3,2-b]furan-3-yl)oxy)propanal (7)

To a solution of 3-(((3R,3aR,6R,6aR)-6-methoxyhexahydrofuro[3,2-b]furan-3-yl)oxy)propan-1-ol (80 mg, 0.37 mmol) in DCM (3 mL) was added DMP (233 mg, 0.55 mmol) at 0° C. and the mixture was stirred at room temperature for 16 hours. The mixture was quenched with aq. Na₂S₂O₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-20% MeOH in DCM) to give the title compound (60 mg, yield 79.3%) as a yellow oil.

### Step 7: N-((1H-pyrrolo[3,2-c]pyridine-2-yl)methyl)-2-(5-((3-(((3R,3aR,6R,6aR)-6-methoxyhexahydrofuro[3,2-b]furan-3-yl)oxy)propyl)amino)-6-oxo-2-phenylpyrimidin-1(6H)-yl)acetamide (Compound 95)

To a mixture of N-((1H-pyrrolo[3,2-c]pyridine-2-yl)methyl)-2-(5-amino-6-oxo-2-phenylpyrimidin-1(6H)-yl)acetamide (30 mg, 0.08 mmol) and 3-(((3R,3aR,6R,6aR)-6-methoxyhexahydrofuro[3,2-b]furan-3-yl)oxy)propanal (50 mg, 0.23 mmol) in MeOH (3 mL) was added NaBH₃CN (15 mg, 0.24 mmol) under N₂ atmosphere and the reaction mixture was stirred at 50° C. for 16 hours. The mixture was diluted with EtOAc, washed with brine, dried over anhydrous Na₂SO₄, filtered, and concentrated under reduced pressure to dryness. The residue was purified by prep-HPLC to give Compound 95 (3.2 mg, yield 7.0%) as a white solid. ¹H NMR (400 MHz, CD₃OD) δ 8.71 (s, 1H), 8.11 (d, J=5.8 Hz, 1H), 7.47 (m, 3H), 7.39 (m, 3H), 7.17 (s, 1H), 6.48 (s, 1H), 4.61 (d, J=7.6 Hz, 4H), 4.54 (s, 2H), 4.07 (m, 1H), 3.98 (m, 3H), 3.78 (m, 1H), 3.68-3.59 (m, 3H), 3.42 (s, 3H), 3.29 (s, 2H), 1.98-1.90 (m, 2H). LC/MS (ESI) m/z: 575 (M+H)⁺. RT (Method A): 0.56 min.

### Scheme 37. Synthesis of N-((1H-pyrrolo[3,2-c]pyridine-2-yl)methyl)-2-(6-oxo-2-(4-(pentafluoro-λ⁶-sulfanyl)phenyl)-5-((3-phenylpropyl)amino)pyrimidin-1(6H)-yl)acetamide (Compound 96)

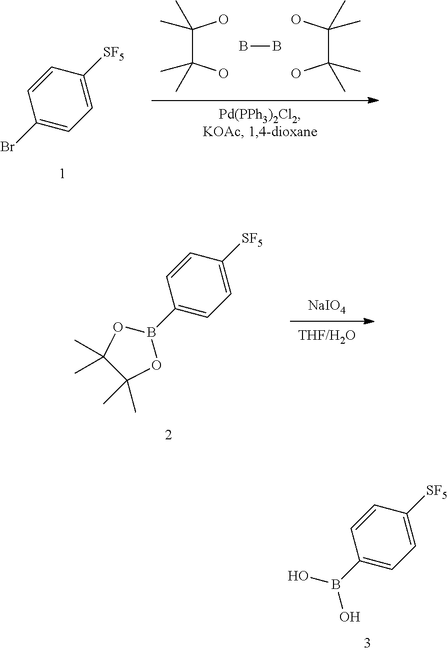

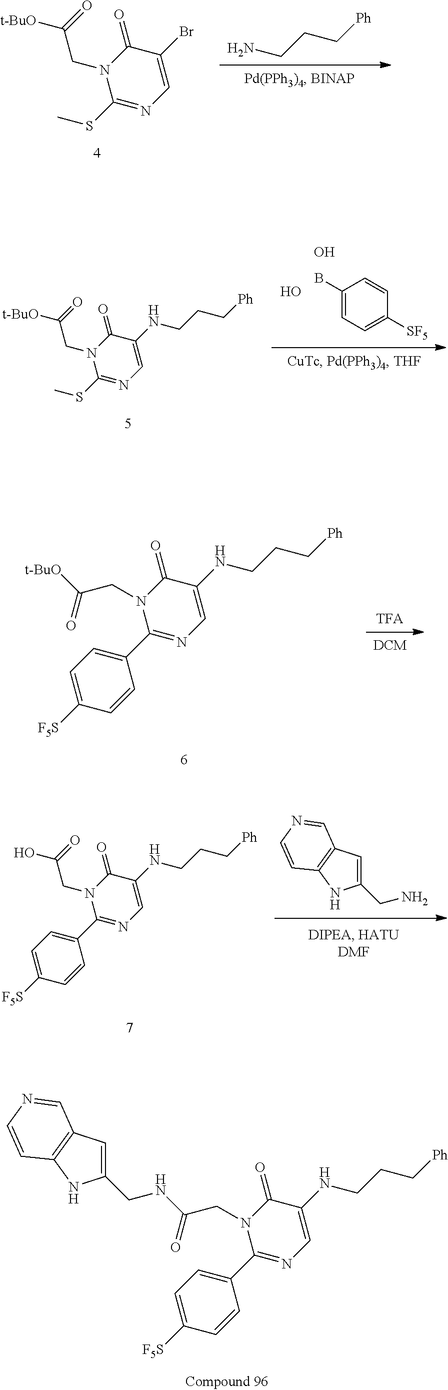

### Step 1: 4,4,5,5-tetramethyl-2-(4-(pentafluoro-λ⁶-sulfanyl)phenyl)-1,3,2-dioxaborolane (2)

To a mixture of (4-bromophenyl)pentafluoro-λ⁶-sulfane (500 mg, 1.78 mmol) and Pin₂B₂ (1.8 g, 7.12 mmol) in 1,4-dioxane (1 mL) was added Pd(PPh₃)₂Cl₂ (385 mg, 0.55 mmol) and KOAc (524 mg, 5.34 mmol) under N₂ atmosphere, the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (300 mg, yield 46.1%) as a white solid. LC/MS (ESI) m/z:331 (M+H)⁺.

### Step 2: (4-(Pentafluoro-λ⁶-sulfanyl)phenyl)boronic acid (3)

To a solution of 4,4,5,5-tetramethyl-2-(4-(pentafluoro-λ⁶-sulfanyl)phenyl)-1,3,2-dioxaborolane (300 mg, 0.91 mmol) in THF (3 mL) and water (3 mL) was added NaIO₄ (27 mg, 0.65 mol), and the mixture was stirred at room temperature for 6 hours. The mixture was concentrated under reduced pressure to dryness. The residue was purified by prep-HPLC to give the title compound as a white solid.

### Step 3: Tert-butyl 2-(2-(methylthio)-6-oxo-5-((3-phenylpropyl)amino)pyrimidin-1(6H)-yl)acetate (3)

To a mixture of tert-butyl 2-(5-bromo-2-(methylthio)-6-oxopyrimidin-1(6H)-yl)acetate (500 mg, 1.49 mmol) and 3-phenylpropan-1-amine (402 mg, 3.0 mmol) in toluene (5 mL) was added Cs₂CO₃ (974 mg, 2.99 mmol), BINAP (187 mg, 0.3 mmol) and Pd(OAc)₂ (34 mg, 0.15 mmol) under N₂ atmosphere, the reaction mixture was degassed under N₂ atmosphere for three times and stirred at 120° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give the title compound (170 mg, yield 29.3%) as a brown oil. LC/MS (ESI) m/z: 390 (M+H)⁺.

### Step 4: Tert-butyl 2-(6-oxo-2-(4-(pentafluoro-λ⁶-sulfanyl)phenyl)-5-((3-phenylpropyl) amino)pyrimidin-1(6H)-yl)acetate (4)

To a mixture of tert-butyl 2-(2-(methylthio)-6-oxo-5-((3-phenylpropyl)amino) pyrimidin-1(6H)-yl)acetate (170 mg, 0.44 mmol) and (4-(pentafluoro-λ⁶-sulfanyl)phenyl)boronic acid (107 mg, 0.43 mmol) in THF (3 mL) was added ((thiophene-2-carbonyl)oxy)copper (185 mg, 0.044 mmol) and Pd(PPh₃)₄ (51 mg, 0.044 mmol) under N₂ atmosphere, the reaction mixture was degassed under N₂ atmosphere for three times and stirred at 55° C. overnight. The mixture was diluted with water and extracted with EtOAc twice. The combined organic layers were washed with saturated aq. NaHCO₃ and brine, dried over anhydrous Na₂SO₄, filtered, and concentrated under reduced pressure to dryness. The residue was purified by flash chromatography (silica gel, 0-20% EtOAc in PE) to give the title compound (55 mg, yield 23.5%) as a brown oil. LC/MS (ESI) m/z: 546 (M+H)⁺.

### Step 5: Tert-butyl 2-(6-oxo-2-(4-(pentafluoro-λ⁶-sulfanyl)phenyl)-5-((3-phenylpropyl) amino)pyrimidin-1(6H)-yl)acetate (5)

To a solution of tert-butyl 2-(6-oxo-2-phenyl-5-((3-phenylpropyl)amino)pyrimidin-1(6H)-yl)acetate (55 mg, 0.10 mmol) in DCM (5 mL) was added TFA (2.5 mL) and the mixture was stirred under N₂ atmosphere at room temperature for 4 hours. The reaction mixture was concentrated under reduced pressure to dryness to give the title compound (46 mg, yield 94.1%) as a yellow solid, which was used directly in the next step without further purification. LC/MS (ESI) m/z: 490 (M+H)⁺.

### Step 6: N-((1H-pyrrolo[3,2-c]pyridine-2-yl)methyl)-2-(6-oxo-2-(4-(pentafluoro-λ⁶-sulfanyl)phenyl)-5-((3-phenylpropyl)amino)pyrimidin-1(6H)-yl)acetamide (Compound 96)

To a mixture of (2S,4R)-1-((9,9-dimethyl-9H-fluorene-3-carbonyl)glycyl)-4-(methylsulfonyl)pyrrolidine-2-carboxylic acid (46 mg, 0.09 mmol) and 1-(1-(phenylsulfonyl)-1H-pyrrolo[3,2-c]pyridine-2-yl)ethan-1-amine hydrochloride (35 mg, 0.1 mmol) in DMF (2 mL) was added DIPEA (50 mg, 0.38 mmol) and HATU (35 mg, 0.09 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=15:1) to give Compound 96 (1.2 mg, yield 2.2%) as a yellow solid. ¹H NMR (400 MHz, CD₃OD) δ 8.75 (s, 1H), 8.14 (d, J=5.7 Hz, 1H), 7.80 (d, J=8.8 Hz, 2H), 7.67 (d, J=8.2 Hz, 2H), 7.47 (d, J=5.1 Hz, 1H), 7.30-7.21 (m, 4H), 7.17 (d, J=7.0 Hz, 1H), 6.56 (s, 1H), 4.58 (d, J=16.9 Hz, 4H), 3.19 (t, J=7.0 Hz, 2H), 2.75 (t, J=7.7 Hz, 2H), 2.05-1.91 (m, 2H). LC/MS (ESI) m/z: 619 (M+H)⁺. RT (Method A): 1.89 min.

### Scheme 38. Synthesis of N-((1H-pyrrolo[3,2-c]pyridine-2-yl)methyl)-2-(5-((3-(4-(oxetan-3-yl-oxy)phenyl)propyl)amino)-6-oxo-2-phenylpyrimidin-1(6H)-yl)acetamide (Compound 103)

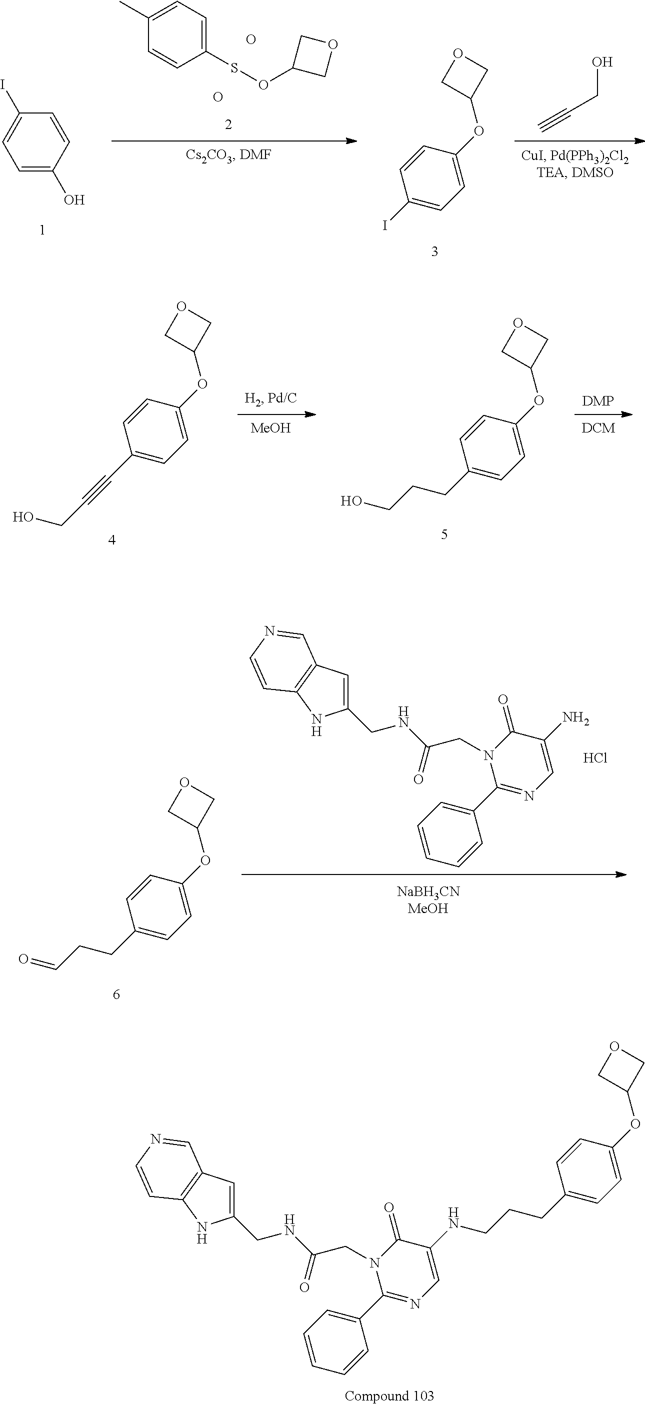

### Step 1: 3-(4-Iodophenoxy)oxetane (2)

To a mixture of oxetan-3-yl 4-methylbenzenesulfonate (3.0 g, 13.6 mmol) and 4-iodophenol (3.1 mg, 13.6 mmol) in DMF (40 mL) was added Cs₂CO₃ (13.3 g, 40.9 mmol) at 0° C. under N₂ atmosphere and the reaction mixture was degassed under N₂ atmosphere for three times and stirred at 70° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-40% EtOAc in PE) to give the title compound (3 g, yield 79.7%) as a white solid. ¹H NMR (400 MHz, CDCl₃) δ 7.57-7.53 (m, 2H), 6.49-6.46 (m, 2H), 5.18-5.12 (m, 1H), 4.95 (t, J=6.9 Hz, 2H), 4.75-4.71 (m, 2H).

### Step 2: 3-(4-(Oxetan-3-yloxy)phenyl)prop-2-yn-1-ol (3)

To a mixture of 3-(4-iodophenoxy)oxetane (713 mg, 2.58 mmol) and prop-2-yn-1-ol (208 mg, 3.85 mmol) in TEA (5 mL) and DMSO (5 mL) was added CuI (49 mg, 0.25 mmol), Pd(PPh₃)₂Cl₂ (178 mg, 0.25 mmol) under N₂ atmosphere, the mixture was degassed under N₂ atmosphere for ten times and stirred under N₂ atmosphere at room temperature 2 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (426 mg, yield 80.8%) as a yellow solid. ¹H NMR (400 MHz, CDCl₃) δ 7.36 (d, J=7.7 Hz, 2H), 6.63 (d, J=7.7 Hz, 2H), 5.24-5.17 (m, 1H), 4.97 (t, J=6.4 Hz, 2H), 4.75 (t, J=5.7 Hz, 2H), 4.48 (s, 2H).

### Step 3: 3-(4-(Oxetan-3-yloxy)phenyl)propan-1-ol (4)

To a solution of 3-(4-(oxetan-3-yloxy)phenyl)prop-2-yn-1-ol (426 mg, 2.01 mmol) in MeOH (4 mL) was added Pd/C (40 mg, 10 wt. %), the mixture was degassed under N₂ atmosphere for three times and stirred under a H₂ balloon at room temperature overnight. The mixture was filtered, and the filtrate was concentrated to dryness to give the title compound (390 mg, yield 58.9%) as a yellow oil, which was used directly in the next step without further purification.

### Step 4: 3-(4-(Oxetan-3-yloxy)phenyl)propanal (5)

To a solution of 3-(4-(oxetan-3-yloxy)phenyl)propan-1-ol (90 mg, 0.43 mmol) in DCM (2 mL) was added DMP (275 mg, 0.65 mmol) under N₂ atmosphere and the reaction mixture was stirred at room temperature for 2 hours. The mixture was diluted with EtOAc, washed with aq. NaHCO₃ solution and brine, dried over anhydrous Na₂SO₄, filtered, and concentrated under reduced pressure to dryness. The residue was purified by prep-TLC (PE:EtOAc=10:1) to give the title compound (49 mg, yield 53.2%). ¹H NMR (400 MHz, CDCl₃) δ 9.83 (t, J=1.4 Hz, 1H), 7.40 (s, 1H), 7.37 (s, 1H), 6.78 (s, 1H), 6.75 (s, 1H), 5.03 (s, 3H), 4.98 (s, 1H), 4.82 (s, 1H), 2.93 (t, J=7.4 Hz, 2H), 2.78 (dd, J=10.6, 3.4 Hz, 2H).

### Step 5: N-((1H-pyrrolo[3,2-c]pyridine-2-yl)methyl)-2-(5-((3-(4-(oxetan-3-yloxy)phenyl) propyl)amino)-6-oxo-2-phenylpyrimidin-1(6H)-yl)acetamide (Compound 103)

To a mixture of 3-(4-(oxetan-3-yloxy)phenyl)propanal (30 mg, 0.14 mmol) and N-((1H-pyrrolo[3,2-c]pyridine-2-yl)methyl)-2-(5-amino-6-oxo-2-phenylpyrimidin-1(6H)-yl) acetamide hydrochloride (26 mg, 0.07 mol) in MeOH (2 mL) was added MgSO₄ (60 mg, 0.49 mmol) and NaBH₃CN (120 mg, 1.9 mmol) under N₂ atmosphere and the reaction mixture was stirred at 50° C. for 2 hours. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by prep-HPLC to give Compound 103 (1 mg, yield 2.5%) as a white solid. ¹H NMR (400 MHz, CD₃OD) δ 8.85 (s, 1H), 8.19 (d, J=6.2 Hz, 1H), 7.61 (d, J=6.0 Hz, 1H), 7.48 (d, J=7.2 Hz, 3H), 7.41 (dd, J=6.6, 2.9 Hz, 2H), 7.26 (d, J=8.6 Hz, 1H), 7.16-7.07 (m, 2H), 6.78 (d, J=8.6 Hz, 1H), 6.66 (d, J=6.4 Hz, 2H), 4.70-4.65 (m, 4H), 4.58 (s, 4H), 4.06 (d, J=5.9 Hz, 1H), 3.15 (t, J=6.9 Hz, 2H), 2.69 (dd, J=9.1, 5.7 Hz, 2H), 2.04-1.91 (m, 2H). LC/MS (ESI) m/z: 565 (M+H)⁺. RT (Method A): 1.30.

The following compounds were prepared based on Scheme 38:

| # | Reactant A | Reactant B | Reactant C | Characterization Data |
|---|---|---|---|---|
| 34^{a,b} | 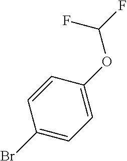 [SMILES: FC(F)Oc1ccc(Br)cc1] | 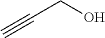 | 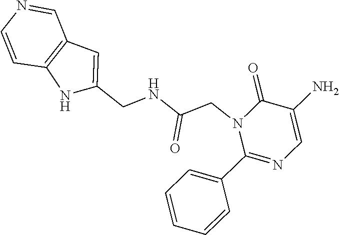 [SMILES: Nc1cnc(-c2ccccc2)n(CC(=O)NCc2cc3cnccc3[nH]2)c1=O] | ¹H NMR (400 MHz, CD₃OD) δ 8.72 (s, 1H), 8.12 (d, J = 5.9 Hz, 1H), 7.42 (ddd, J = 17.6, 14.5, 7.9 Hz, 6H), 7.25 (d, J = 8.4 Hz, 2H), 7.09-7.04 (m, 3H), 6.75 (t, J = 74.5 Hz, 1H), 6.50 (s, 1H), 4.62 (s, 2H), 4.54 (s, 2H), 3.17 (t, J = 6.9 Hz, 2H), 2.74 (t, J = 7.6 Hz, 2H), 2.01-1.93 (m, 2H). 1.51 LC/MS (ESI) m/z: 559 (M + H)⁺. RT (Method A): 1.51 min. |
| 39^{a} | 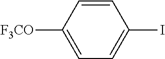 [SMILES: FC(F)(F)Oc1ccc(I)cc1] | 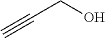 | 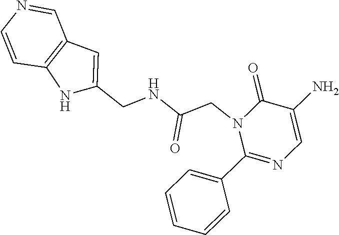 [SMILES: Nc1cnc(-c2ccccc2)n(CC(=O)NCc2cc3cnccc3[nH]2)c1=O] | ¹H NMR (400 MHz, CD₃OD) δ 8.83 (s, 1H), 8.18 (d, J = 6.1 Hz, 1H), 7.58 (d, J = 6.2 Hz, 1H), 7.48 (t, J = 6.1 Hz, 3H), 7.42 (d, J = 7.0 Hz, 2H), 7.33 (d, J = 8.6 Hz, 2H), 7.18 (d, J = 8.0 Hz, 2H), 7.12 (s, 1H), 6.64 (s, 1H), 4.64 (s, 2H), 4.58 (s, 2H), 3.19 (d, J = 7.0 Hz, 2H), 2.81-2.77 (m, 2H), 2.03-1.98 (m, 2H). LC/MS (ESI) m/z: 577 (M + H)⁺. RT (Method A): 1.15 min. |
| 40^{a} | 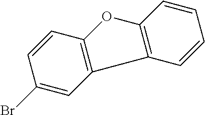 [SMILES: Brc1ccc2oc3ccccc3c2c1] | 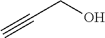 | 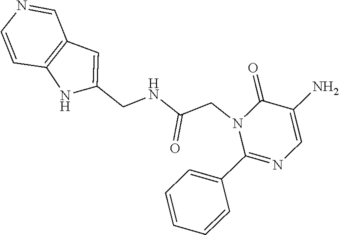 [SMILES: Nc1cnc(-c2ccccc2)n(CC(=O)NCc2cc3cnccc3[nH]2)c1=O] | ¹H NMR (400 MHz, CD₃OD) δ 8.74 (s, 1H), 8.12 (d, J = 5.9 Hz, 1H), 8.00 (d, J = 7.4 Hz, 1H), 7.89 (s, 1H), 7.55 (d, J = 8.3 Hz, 1H), 7.50 (d, J = 8.4 Hz, 1H), 7.45 (dd, J = 8.0, 5.3 Hz, 5H), 7.37 (ddd, J = 11.2, 9.3, 7.3 Hz, 4H), 7.10 (s, 1H), 6.52 (s, 1H), 4.60 (s, 2H), 4.55 (s, 2H), 3.24 (t, J = 6.9 Hz, 2H), 2.94 (t, J = 7.5 Hz, 2H), 2.11 (dd, J = 14.9, 7.1 Hz, 2H). LC/MS (ESI) m/z: 583 (M + H)⁺. RT (Method A): 1.81 min. |
| 41ª | 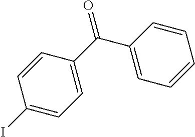 [SMILES: O=C(c1ccccc1)c1ccc(I)cc1] | 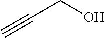 | 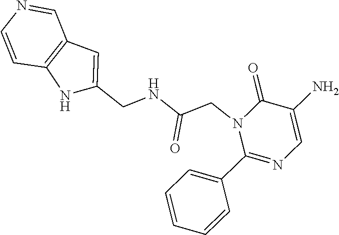 [SMILES: Nc1cnc(-c2ccccc2)n(CC(=O)NCc2cc3cnccc3[nH]2)c1=O] | ¹H NMR (400 MHz, CD₃OD) δ 8.70 (s, 1H), 8.10 (d, J = 5.8 Hz, 1H), 7.74 (dd, J = 8.5, 4.8 Hz, 4H), 7.62 (d, J = 7.5 Hz, 1H), 7.52 (d, J = 7.8 Hz, 2H), 7.50-7.45 (m, 3H), 7.43 (s, 1H), 7.39 (dd, J = 9.8, 3.5 Hz, 4H), 7.11 (s, 1H), 6.47 (s, 1H), 4.62 (s, 2H), 4.53 (s, 2H), 3.24 (t, J = 6.8 Hz, 2H), 2.87 (t, J = 7.6 Hz, 2H), 2.11-2.02 (m, 2H). LC/MS (ESI) m/z: 597 (M + H)⁺. RT (Method A): 1.63 min. |
| 45^{a,b} | 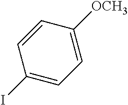 | 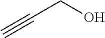 | 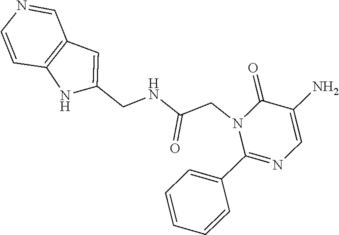 [SMILES: Nc1cnc(-c2ccccc2)n(CC(=O)NCc2cc3cnccc3[nH]2)c1=O] | ¹H NMR (400 MHz, CD₃OD) δ 8.70 (s, 1H), 8.11 (d, J = 4.9 Hz, 1H), 7.46 (t, J = 7.1 Hz, 3H), 7.38 (t, J = 7.0 Hz, 3H), 7.13 (d, J = 7.5 Hz, 2H), 7.07 (s, 1H), 6.84 (d, J = 7.7 Hz, 2H), 6.48 (s, 1H), 4.62 (s, 2H), 4.54 (s, 2H), 3.76 (s, 3H), 3.16 (t, J = 6.8 Hz, 2H), 2.69 (t, J = 7.3 Hz, 2H), 2.00-1.92 (m, 2H). LC/MS (ESI) m/z: 523 (M + H)⁺. RT (Method A): 1.45 min. |
| 64^{a,b} | 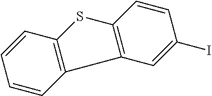 [SMILES: Ic1ccc2sc3ccccc3c2c1] | 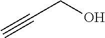 | 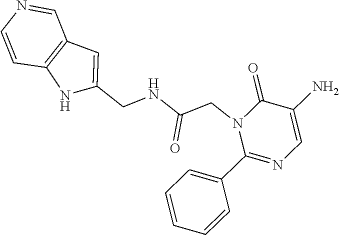 [SMILES: Nc1cnc(-c2ccccc2)n(CC(=O)NCc2cc3cnccc3[nH]2)c1=O] | ¹H NMR (400 MHz, CD₃OD) δ 8.71 (d, J = 0.8 Hz, 1H), 8.23-8.19 (m, 1H), 8.11 (d, J = 5.9 Hz, 2H), 7.85 (d, J = 5.7 Hz, 1H), 7.79 (d, J = 8.2 Hz, 1H), 7.46-7.41 (m, 5H), 7.40 (d, J = 5.8 Hz, 2H), 7.36 (d, J = 7.4 Hz, 2H), 7.10 (s, 1H), 6.49 (s, 1H), 4.59 (s, 2H), 4.54 (s, 2H), 3.24 (d, J = 6.8 Hz, 2H), 2.95 (d, J = 7.6 Hz, 2H), 2.14-2.09 (m, 2H). LC/MS (ESI) m/z: 599 (M + H)⁺. |
| | | | | RT (Method A): 2.06 min. |
| 77^{a,c} | 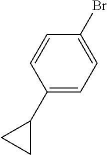 | 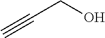 | 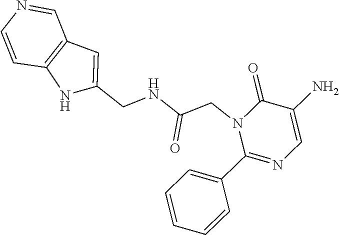 [SMILES: Nc1cnc(-c2ccccc2)n(CC(=O)NCc2cc3cnccc3[nH]2)c1=O] | ¹H NMR (400 MHz, CD₃OD) δ 8.89 (s, 1H), 8.47 (s, 1H), 8.21 (d, J = 6.3 Hz, 1H), 7.67 (d, J = 6.3 Hz, 1H), 7.48 (t, J = 6.3 Hz, 3H), 7.43-7.39 (m, 2H), 7.09 (d, J = 7.6 Hz, 3H), 6.99 (d, J = 8.1 Hz, 2H), 6.71 (s, 1H), 4.64 (s, 2H), 4.59 (s, 2H), 3.16 (t, J = 7.0 Hz, 2H), 2.69 (t, J = 7.5 Hz, 2H), 1.99-1.92 (m, 2H), 1.89-1.83 (m, 1H), 0.93-0.88 (m, 2H), 0.64-0.60 (m, 2H). LC/MS (ESI) m/z: 533 (M + H)⁺. |
| | | | | RT (Method A): 1.76 min. |
| 104 | 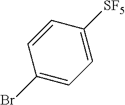 | 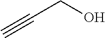 | 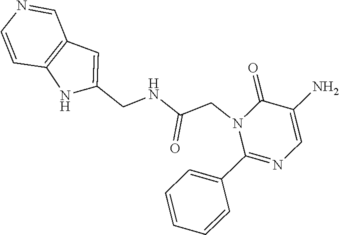 [SMILES: Nc1cnc(-c2ccccc2)n(CC(=O)NCc2cc3cnccc3[nH]2)c1=O] | ¹H NMR (400 MHz, CD₃OD) δ 8.72 (s, 1H), 8.12 (d, J = 5.9 Hz, 1H), 7.72 (d, J = 8.7 Hz, 2H), 7.44 (dt, J = 23.5, 7.5 Hz, 8H), 7.12 (s, 1H), 6.50 (s, 1H), 4.62 (s, 2H), 4.55 (s, 2H), 3.21 (t, J = 6.9 Hz, 2H), 2.88-2.81 (m, 2H), 2.08-1.99 (m, 2H). LC/MS (ESI) m/z: 619 (M + H)⁺. RT (Method A): 1.88 min. |

^{a}Steps 2-5 only.
^{b}PCC was used in place of DMP in Step 4.
^{c}Step 2 was performed with Pd(PPh₃)₄ and CuI in the presence of DBU in THF.

### Scheme 39. Synthesis of N-((1H-pyrrolo[3,2-c]pyridine-2-yl)methyl)-2-(5-((3-(4-(S-methylsulfonimidoyl)phenyl)propyl)amino)-6-oxo-2-phenylpyrimidin-1(6H)-yl) acetamide (Compound 105)

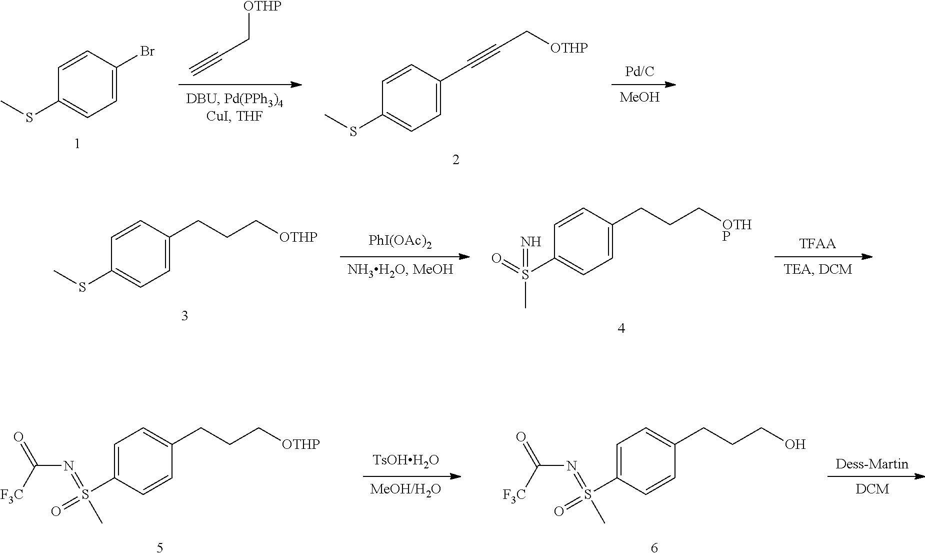

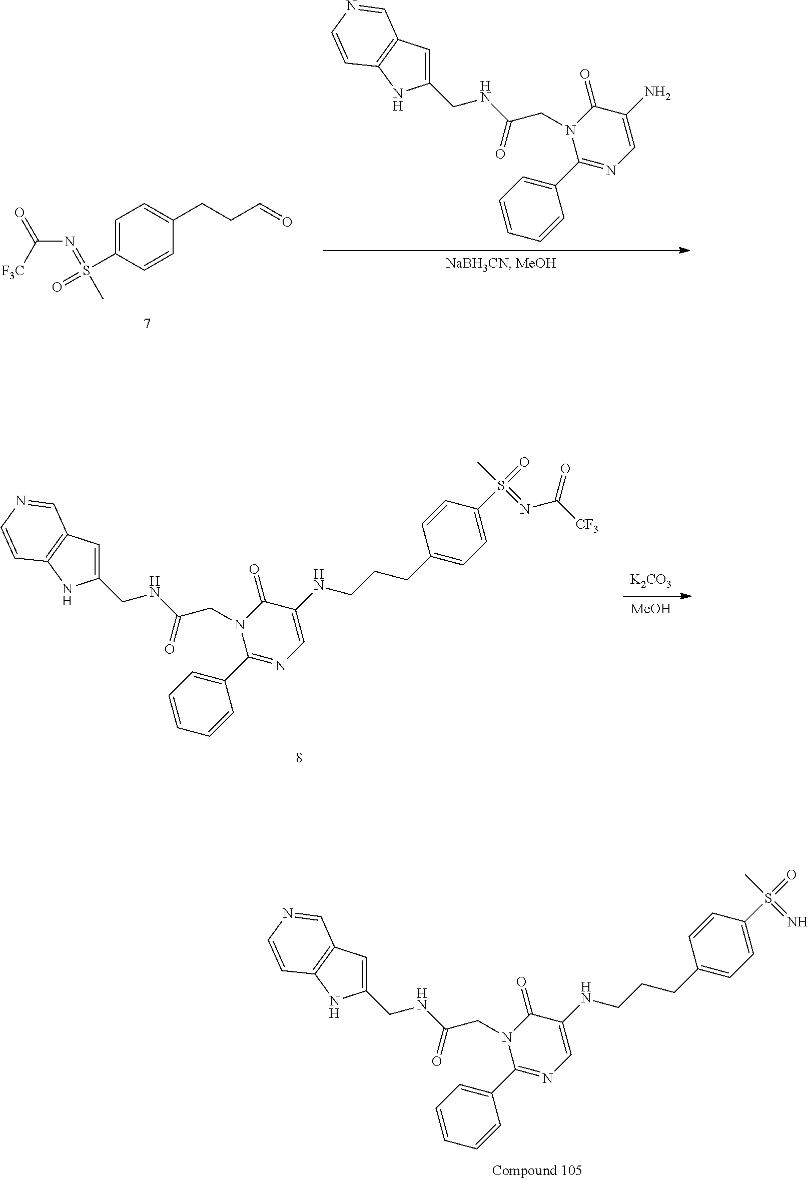

### Step 1: 2-((3-(4-(Methylthio)phenyl)prop-2-yn-1-yl)oxy)tetrahydro-2H-pyran (2)

To a solution of 6-methylbenzo[b]thiophene-2-carboxylic acid (3.0 g, 14.7 mmol) in THF (30 mL) was added 2-(prop-2-yn-1-yloxy)tetrahydro-2H-pyran (2.5 g, 17.7 mmol) followed by DBU (2.7 g, 17.7 mmol), CuI (84 mg, 0.44 mmol) and Pd(PPh₃)₄ (515 mg, 0.45 mmol) under N₂ atmosphere. The mixture degassed under N₂ atmosphere for three times and stirred at 80° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-6% EtOAc in PE) to give the title compound (3.8 g, yield 98.0%) as a colorless oil. LC/MS (ESI) m/z: 263 (M+H)⁺.

### Step 2: 2-(3-(4-(Methylthio)phenyl)propoxy)tetrahydro-2H-pyran (3)

To a solution of 2-((3-(4-(methylthio)phenyl)prop-2-yn-1-yl)oxy)tetrahydro-2H-pyran (4.0 g, 1.08 mmol) in MeOH (4 mL) was added Pd/C (400 mg, 10 wt. %), the mixture was degassed under N₂ atmosphere for three times and stirred under a H₂ balloon at 0° C. for 16 hours. The mixture was filtered, and the filtrate was concentrated under reduced pressure to give the title compound (3.4 g, yield 88.1%) as a yellow oil, which was directly used in the next step without further purification. ¹H NMR (400 MHz, CDCl₃) δ 7.21-7.18 (m, 2H), 7.15-7.10 (m, 2H), 4.59-4.54 (m, 1H), 3.90-3.84 (m, 1H), 3.80-3.73 (m, 1H), 3.52-3.47 (m, 1H), 3.43-3.36 (m, 1H), 2.71-2.63 (m, 2H), 2.47 (d, J=5.4 Hz, 3H), 1.94-1.88 (m, 2H), 1.87-1.80 (m, 1H), 1.76-1.70 (m, 1H), 1.62-1.52 (m, 4H).

### Step 3: Imino(methyl)(4-(3-((tetrahydro-2H-pyran-2-yl)oxy)propyl)phenyl)-l6-sulfanone (4)

To a solution of 2-(3-(4-(methylthio)phenyl)propoxy)tetrahydro-2H-pyran (800 mg, 3.00 mol) in MeOH (10 mL) was added NH₃·H₂O (2.0 mL, 25 wt. % in water) followed by PhI(OAc)₂ (2.9 g, 9.03 mmol) at 0° C. and the mixture was stirred at room temperature overnight. The mixture was diluted with EtOAc, washed with saturated aq. Na₂S₂O₃ solution and brine, dried over anhydrous Na₂SO₄, filtered, and concentrated under reduced pressure to dryness. The residue was purified by flash chromatography (silica gel, 0-100% EtOAc in PE) to give the title compound (700 mg, yield 78.3%) as a colorless oil. LC/MS (ESI) m/z: 298 (M+H)⁺.

### Step 4: 2,2,2-Trifluoro-N-(methyl(oxo)(4-(3-((tetrahydro-2H-pyran-2-yl)oxy)propyl) phenyl)-l6-sulfanylidene)acetamide (5)

To a solution of imino(methyl)(4-(3-((tetrahydro-2H-pyran-2-yl)oxy)propyl)phenyl)-l6-sulfanone (300 mg, 1.01 mmol) in DCM (4 mL) was added TEA (310 mg, 3.07 mmol) followed by TFAA (430 mg, 2.05 mmol) at 0° C. and the mixture was stirred at room temperature for 3 hours. The mixture was diluted with DCM, washed with water and brine, dried over anhydrous Na₂SO₄, filtered, and concentrated under reduced pressure to give the title compound (200 mg, yield 50.4%) as a yellow solid, which was directly used in the next step without further purification. ¹H NMR (400 MHz, DMSO-d₆) δ 7.93 (d, J=8.4 Hz, 2H), 7.59 (d, J=8.4 Hz, 2H), 4.54 (d, J=3.8 Hz, 1H), 3.75 (s, 3H), 3.74-3.68 (m, 1H), 3.67-3.61 (m, 1H), 3.44-3.39 (m, 1H), 3.36 (dd, J=6.4, 3.5 Hz, 1H), 2.78 (dd, J=8.3, 5.7 Hz, 2H), 1.88 (dd, J=14.5, 7.0 Hz, 2H), 1.71 (d, J=9.0 Hz, 1H), 1.61 (dd, J=9.0, 2.7 Hz, 1H), 1.45 (dd, J=7.4, 4.0 Hz, 4H).

### Step 5: 2,2,2-Trifluoro-N-((4-(3-hydroxypropyl)phenyl)(methyl)(oxo)-l6-sulfanylidene)acetamide (6)

To a solution of 2,2,2-trifluoro-N-(methyl(oxo)(4-(3-((tetrahydro-2H-pyran-2-yl)oxy)propyl)phenyl)-16-sulfanylidene)acetamide (200 mg, 0.51 mmol) in MeOH (2.4 mL) and water (0.6 mL) was added TsOH·H₂O (145 mg, 0.76 mmol) at 0° C. and the mixture was stirred at room temperature for 3 hours. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-100% EtOAc in PE) to give the title compound (150 mg, yield 95.3%) as a colorless oil. LC/MS (ESI) m/z: 310(M+H)⁺. ¹H NMR (400 MHz, DMSO-d₆) δ 7.80 (d, J=8.3 Hz, 2H), 7.47 (d, J=8.4 Hz, 2H), 4.26 (t, J=6.4 Hz, 2H), 3.60 (s, 3H), 3.40 (d, J=18.5 Hz, 1H), 2.73-2.63 (m, 2H), 1.97-1.86 (m, 2H).

### Step 6: 2,2,2-Trifluoro-N-(methyl(oxo)(4-(3-oxopropyl)phenyl)-l6-sulfanylidene) acetamide (7)

To a solution of 2,2,2-trifluoro-N-((4-(3-hydroxypropyl)phenyl)(methyl)(oxo)-l6-sulfanylidene)acetamide (80 mg, 0.26 mmol) in DCM (2 mL) was added Dess-Martin periodinane (165 mg, 0.39 mmol) at 0° C. and the mixture was stirred at room temperature for 2 hours. The mixture was filtered and concentrated under reduced pressure to dryness. The residue was purified by flash chromatography (silica gel, 0-96% EtOAc in PE) to give the title compound (65 mg, yield 81.7%) as a colorless oil. LC/MS (ESI) m/z: 308 (M+H)⁺.

### Step 7: N-((4-(3-((1-(2-(((1H-pyrrolo[3,2-c]pyridine-2-yl)methyl)amino)-2-oxoethyl)-6-oxo-2-phenyl-1,6-dihydropyrimidin-5-yl)amino)propyl)phenyl)(methyl)(oxo)-l6-sulfanylidene)-2,2,2-trifluoroacetamide (7)

To a mixture of N-((1H-pyrrolo[3,2-c]pyridine-2-yl)methyl)-2-(5-amino-6-oxo-2-phenylpyrimidin-1(6H)-yl)acetamide (50 mg, 0.13. mmol) and 2,2,2-trifluoro-N-(methyl(oxo)(4-(3-oxopropyl)phenyl)-l6-sulfanylidene)acetamide (62 mg, 0.20 mmol) in MeOH (3 mL) was added NaBH₃CN (25 mg, 0.40 mmol) under N₂ atmosphere and the reaction mixture was stirred at 50° C. for 3 hours. The mixture was diluted with EtOAc, washed with brine, dried over anhydrous Na₂SO₄, filtered, and concentrated under reduced pressure to give the title compound (60 mg, yield 67.4%) as a white solid, which was directly used in the next step without further purification. LC/MS (ESI) m/z: 666 (M+H)⁺.

### Step 8: N-((1H-pyrrolo[3,2-c]pyridine-2-yl)methyl)-2-(5-((3-(4-(S-methylsulfonimidoyl) phenyl)propyl)amino)-6-oxo-2-phenylpyrimidin-1(6H)-yl)acetamide (Compound 105)

To a solution of N-((4-(3-((1-(2-(((1H-pyrrolo[3,2-c]pyridine-2-yl)methyl)amino)-2-oxoethyl)-6-oxo-2-phenyl-1,6-dihydropyrimidin-5-yl)amino)propyl)phenyl)(methyl) (oxo)-l6-sulfanylidene)-2,2,2-trifluoroacetamide (50 mg, 0.075 mol) in MeOH (2 mL) was added K₂CO₃ (30 mg, 0.22 mmol) and the mixture was stirred at room temperature for 3 hours. The mixture was concentrated under reduced pressure to dryness. The residue was purified by prep-HPLC to give Compound 105 (6.1 mg, yield 14.2%) as a white solid. ¹H NMR (400 MHz, CD₃OD) δ 8.92 (s, 1H), 8.24 (d, J=6.3 Hz, 1H), 7.94 (d, J=8.4 Hz, 2H), 7.71 (d, J=6.3 Hz, 1H), 7.53-7.49 (m, 5H), 7.45-7.42 (m, 2H), 7.11 (s, 1H), 6.74 (s, 1H), 4.65 (s, 2H), 4.61 (s, 2H), 3.24 (t, J=6.9 Hz, 2H), 3.15 (s, 3H), 2.89 (t, J=7.6 Hz, 2H), 2.09-2.02 (m, 2H). LC/MS (ESI) m/z: 570 (M+H)⁺. RT (Method A): 0.80 min.

### Scheme 40. Synthesis of 2-(5-((3-(1,4(1,4)-dibenzenacyclohexaphane-1²-yl)propyl)amino)-6-oxo-2-phenylpyrimidin-1(6H)-yl)-N-((1H-pyrrolo[3,2-c]pyridine-2-yl)methyl)acetamide (Compound 113)

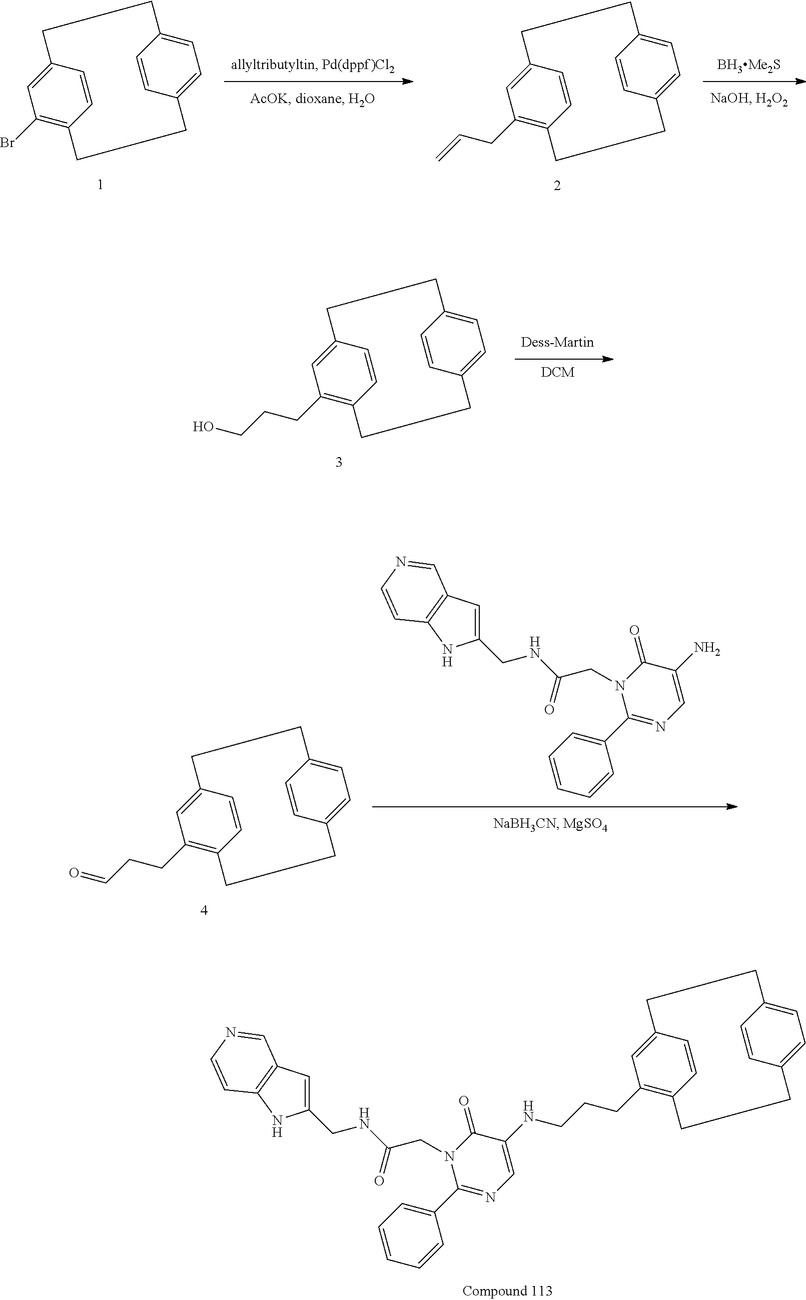

### Step 1: 1²-Allyl-1,4(1,4)-dibenzenacyclohexaphane (2)

To a mixture of 1²-bromo-1,4(1,4)-dibenzenacyclohexaphane (100 mg, 0.35 mmol) and allyltributyltin (173 mg, 0.53 mmol) in DMF (2 mL) was added Pd(PPh₃)₄ (20 mg, 0.017 mmol) under N₂ atmosphere at 0° C. The mixture was degassed under N₂ atmosphere for three times and stirred at 100° C. overnight. The residue was purified by flash chromatography (silica gel, 0-10% EtOAc in PE) to give the title compound (71 mg, yield 81.9%) as a white solid.

### Step 2: 3-(1,4(1,4)-Dibenzenacyclohexaphane-1²-yl)propan-1-ol (3)

To a solution of 1²-allyl-1,4(1,4)-dibenzenacyclohexaphane (71 mg, 0.28 mmol) in THF (2 mL) was added BH₃·Me₂S (1.0 mL, 1.0 mmol) drop-wisely at 0° C. and the reaction solution was stirred at 0° C. for 2 hours. To the above mixture, ethanol (1.4 mL) was added at 0° C. followed by drop-wise addition of aq. NaOH solution (3M, 0.4 mL) and H₂O₂ (0.8 mL, 30% wt.) successively at −10° C. The resulting mixture was stirred at room temperature for 3 hours. The layers were separated, and the aqueous layer was extracted with MTBE twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (36 mg, yield 47.3%) as a colorless oil.

### Step 3: 3-(1,4(1,4)-Dibenzenacyclohexaphane-1²-yl)propanal (4)

To a solution of 3-(1,4(1,4)-dibenzenacyclohexaphane-1²-yl)propan-1-ol (36 mg, 0.14 mmol) in DCM (5 mL) was added Dess-Martin periodinane (85 mg, 0.20 mmol) under N₂ atmosphere and the reaction mixture was stirred at room temperature for 2 hours. The mixture was diluted with EtOAc, washed with aq. Na₂SO₃ solution and brine, dried over anhydrous Na₂SO₄, filtered, and concentrated under reduced pressure to dryness. The residue was purified by flash chromatography (silica gel, 0-10% MeOH in DCM) to give the title compound (21 mg, yield 58.9%) as a white solid.

### Step 4: 2-(5-((3-(1,4(1,4)-Dibenzenacyclohexaphane-1²-yl)propyl)amino)-6-oxo-2-phenylpyrimidin-1(6H)-yl)-N-((1H-pyrrolo[3,2-c]pyridine-2-yl)methyl)acetamide (Compound 113)

To a mixture of 3-(1,4(1,4)-dibenzenacyclohexaphane-1²-yl)propanal (21 mg, 0.08 mmol) and N-((1H-pyrrolo[3,2-c]pyridine-2-yl)methyl)-2-(5-amino-6-oxo-2-phenylpyrimidin-1(6H)-yl)acetamide (28 mg, 0.08 mmol) in MeOH (2 mL) was added NaBH₃CN (38 mg, 0.63 mmol) under N₂ atmosphere and the reaction mixture was stirred at 50° C. for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=10:1) to give Compound 113 (1.5 mg, yield 3.34%) as a white solid. ¹H NMR (400 MHz, CD₃OD) δ 8.72 (s, 1H), 8.11 (d, J=5.9 Hz, 1H), 7.49-7.33 (m, 6H), 7.02 (s, 1H), 6.67 (d, J=7.9 Hz, 1H), 6.51-6.32 (m, 6H), 6.17 (s, 1H), 4.58 (d, J=27.6 Hz, 4H), 3.38 (d, J=11.2 Hz, 1H), 3.13-2.94 (m, 8H), 2.79 (dd, J=15.1, 8.5 Hz, 2H), 2.48-2.36 (m, 1H), 1.87-1.73 (m, 2H). LC/MS (ESI) m/z: 623 (M+H)⁺.

### Scheme 41. Synthesis of N-((1H-pyrrolo[3,2-c]pyridine-2-yl)methyl)-2-(2-(4-(N-cyano-S-methylsulfonimidoyl)phenyl)-6-oxo-5-((3-phenylpropyl)amino)pyrimidin-1(6H)-yl)acetamide (Compound 114)

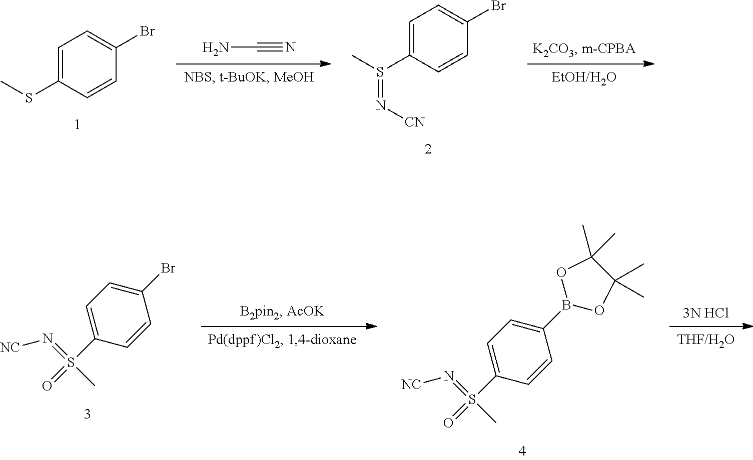

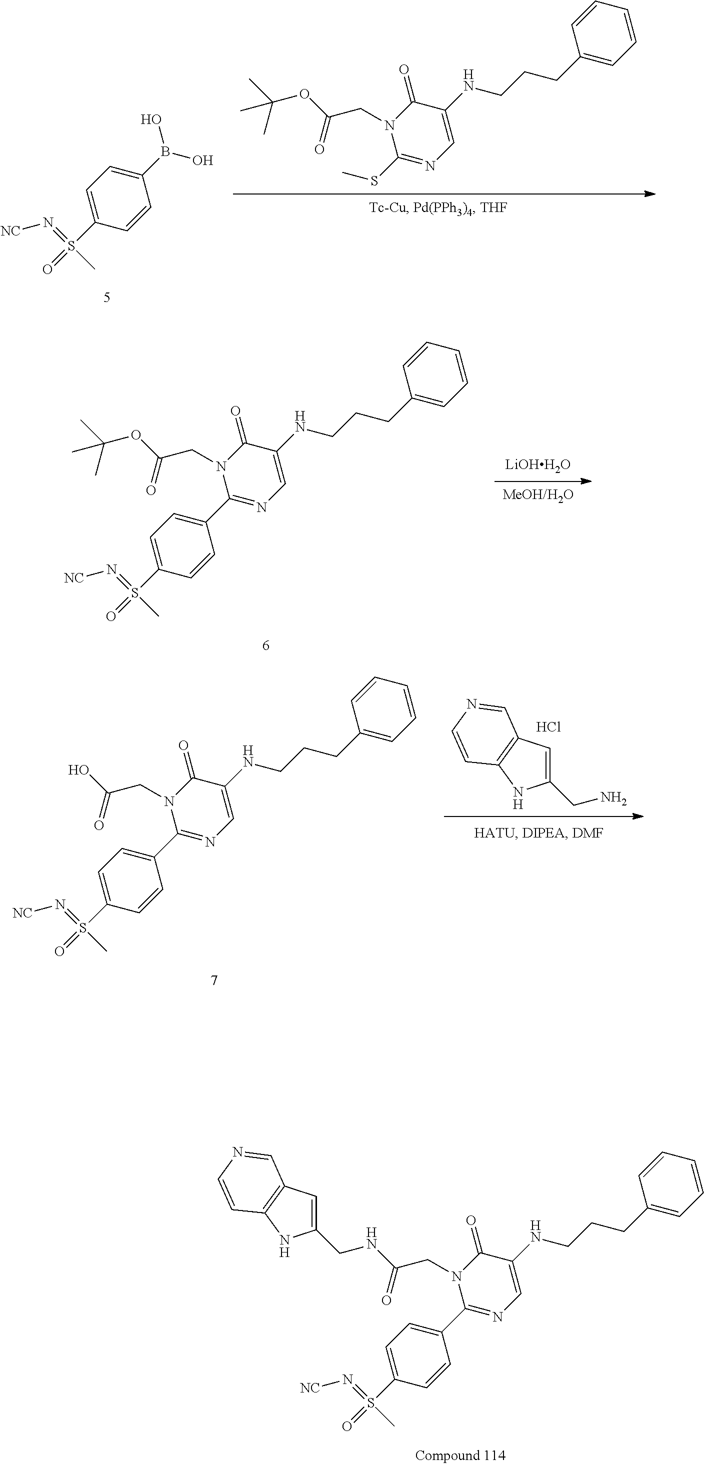

### Step 1: (Z)—N-((4-bromophenyl)(methyl)-λ⁴-sulfanylidene)cyanamide (2)

To a mixture of (4-bromophenyl)(methyl)sulfane (5.0 g, 24.6 mmol), cyanamide (1.35 g, 32.0 mmol) and NBS (6.57 g, 36.9 mmol) in MeOH (50 mL) was added t-BuOK (29.5 mL, 29.5 mmol, 1 M in THF) drop-wisely at 0° C. and the mixture was stirred at room temperature for 16 hours. The mixture was diluted with water and extracted with DCM twice. The combined organic layers were washed with brine, dried over anhydrous Na₂SO₄, filtered, and concentrated to dryness. The residue was purified by flash chromatography (silica gel, 0-50% EtOAc in PE) to give title compound (4.0 g, yield 66.9%) as a colorless oil. LC/MS (ESI) m/z: 243 (M+H)⁺.

### Step 2: N-((4-bromophenyl)(methyl)(oxo)-λ⁶-sulfanylidene)cyanamide (3)

To a mixture of (Z)—N-((4-bromophenyl)(methyl)-λ⁴-sulfanylidene)cyanamide (4.0 g, 16.5 mmol) and K₂CO₃ (6.57 g, 49.4 mmol) in EtOH (30 mL) and water (10 mL) was added m-CPBA (4.3 g, 24.7 mmol) in portions at 0° C. and the mixture was stirred at room temperature for 16 hours. The mixture was quenched with saturated aq. Na₂S₂O₃ solution and extracted with DCM twice. The combined organic layers were washed with saturated aq. NaHCO₃ solution and brine, dried over anhydrous Na₂SO₄, filtered, and concentrated to dryness. The residue was purified by flash chromatography (silica gel, 0-10% MeOH in DCM) to give title compound (2.3 g, yield 53.5%) as a white solid. LC/MS (ESI) m/z: 259 (M+H)⁺.

### Step 3: N-(methyl(oxo)(4-(4,4,5,5-tetramethyl-1,3,2-dioxaborolan-2-yl)phenyl)-λ⁶-sulfanylidene)cyanamide (4)

To a mixture of N-((4-bromophenyl)(methyl)(oxo)-λ⁶-sulfanylidene)cyanamide (2.3 g, 8.91 mmol) and bis(pinacolato)diboron (3.4 g, 13.4 mmol) in 1,4-dioxane (23 mL) was added AcOK (2.6 g, 26.7 mmol) and Pd(dppf)Cl₂ (651 mg, 0.89 mmol) under N₂ atmosphere, the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give title compound (2.0 g, yield 49.3%) as a white solid. LC/MS (ESI) m/z: 307 (M+H)⁺.

### Step 4: (4-(N-cyano-S-methylsulfonimidoyl)phenyl)boronic acid (5)

To a solution of N-(methyl(oxo)(4-(4,4,5,5-tetramethyl-1,3,2-dioxaborolan-2-yl)phenyl)-λ⁶-sulfanylidene)cyanamide (2.0 g, 6.51 mmol) in THF (16 mL) and water (4 mL) was added 3 N aq. HCl (4 mL), and the mixture was stirred at room temperature overnight. The reaction mixture was concentrated under reduced pressure to dryness to give title compound (1.3 g, yield 89.0%) as a white solid, which was used directly in the next step without further purification. LC/MS (ESI) m/z: 225 (M+H)⁺.

### Step 5: Tert-butyl 2-(2-(4-(N-cyano-S-methylsulfonimidoyl)phenyl)-6-oxo-5-((3-phenylpropyl)amino)pyrimidin-1(6H)-yl)acetate (6)

To a mixture of (4-(N-cyano-S-methylsulfonimidoyl)phenyl)boronic acid (300 mg, 1.34 mmol) and tert-butyl 2-(2-(methylthio)-6-oxo-5-((3-phenylpropyl)amino)pyrimidin-1(6H)-yl)acetate (260 mg, 0.67 mmol) in THF (3 mL) was added CuTc (281 mg, 1.47 mmol) and Pd(PPh₃)₄ (155 mg, 0.13 mmol) under N₂ atmosphere, the reaction mixture was degassed under N₂ atmosphere for three times and stirred at 80° C. overnight. The mixture was diluted with water and extracted with EtOAc twice. The combined organic layers were washed with saturated aq. NaHCO₃ solution and brine, dried over anhydrous Na₂SO₄, filtered, and concentrated under reduced pressure to dryness. The residue was purified by flash chromatography (silica gel, 0-50% EtOAc in PE) to give title compound (69 mg, yield 19.8%) as a brown oil. LC/MS (ESI) m/z: 522 (M+H)⁺.

### Step 6: 2-(2-(4-(N-cyano-S-methylsulfonimidoyl)phenyl)-6-oxo-5-((3-phenylpropyl) amino)pyrimidin-1(6H)-yl)acetic acid (7)

To a solution of tert-butyl 2-(2-(4-(N-cyano-S-methylsulfonimidoyl)phenyl)-6-oxo-5-((3-phenylpropyl)amino)pyrimidin-1(6H)-yl)acetate (30 mg, 0.058 mmol) in MeOH/H₂O (1 mL, v/v=4/1) was added LiOH·H₂O (5 mg, 0.12 mmol) and the mixture was stirred at room temperature for 2 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give title compound (10 mg, yield 37.0%) as a brown solid, which was used directly in the next step without further purification. LC/MS (ESI) m/z: 466 (M+H)⁺.

### Step 7: N-((1H-pyrrolo[3,2-c]pyridine-2-yl)methyl)-2-(2-(4-(N-cyano-S-methylsulfonimidoyl)phenyl)-6-oxo-5-((3-phenylpropyl)amino)pyrimidin-1(6H)-yl)acetamide (Compound 114)

To a mixture of 2-(2-(4-(N-cyano-S-methylsulfonimidoyl)phenyl)-6-oxo-5-((3-phenylpropyl)amino)pyrimidin-1(6H)-yl)acetic acid (10 mg, 0.021 mmol) and (1H-pyrrolo[3,2-c]pyridine-2-yl)methanamine hydrochloride (8 mg, 0.042 mmol) in DMF (1 mL) was added DIPEA (14 mg, 0.11 mmol) and HATU (12 mg, 0.032 mmol) under N₂ atmosphere and the reaction mixture was stirred at room temperature for 2 hours. The mixture was concentrated under reduced pressure to dryness. The residue was purified by prep-HPLC to give Compound 114 (1.0 mg, yield 8.1%) as a white solid. ¹H NMR (400 MHz, CD₃OD) δ 8.97 (s, 1H), 8.42-8.39 (m, 1H), 8.26 (d, J=6.3 Hz, 1H), 8.07 (d, J=8.5 Hz, 2H), 7.88 (d, J=8.6 Hz, 2H), 7.78 (d, J=6.6 Hz, 1H), 7.27 (d, J=7.2 Hz, 2H), 7.22 (d, J=6.8 Hz, 2H), 7.18 (d, J=7.0 Hz, 1H), 7.13 (s, 1H), 6.78 (s, 1H), 4.67 (d, J=10.0 Hz, 4H), 3.58 (s, 3H), 3.20 (t, J=7.0 Hz, 2H), 2.77-2.73 (m, 2H), 2.02-1.98 (m, 2H). LC/MS (ESI) m/z: 595 (M+H)⁺. RT (Method A): 1.38 min.

### Scheme 42. Synthesis of N-((1H-pyrrolo[3,2-c]pyridine-2-yl)methyl)-2-(5-((4-cyanophenethyl)amino)-6-oxo-2-phenylpyrimidin-1(6H)-yl)acetamide (Compound 28)

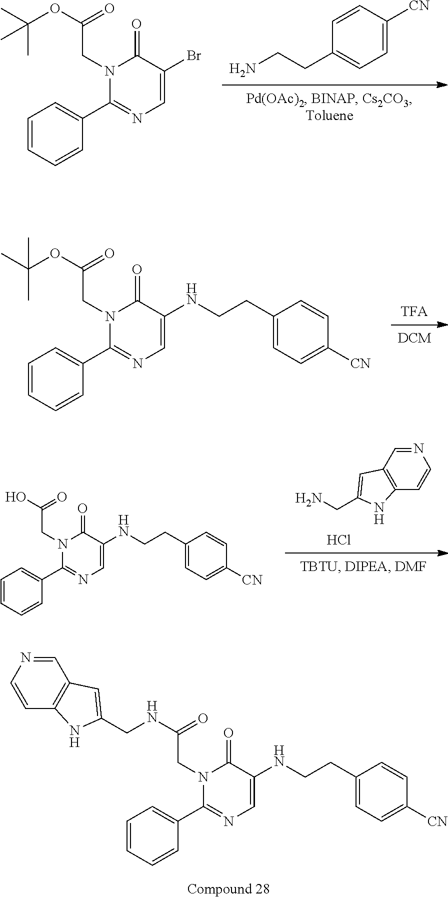

### Step 1: tert-butyl 2-(5-((4-cyanophenethyl)amino)-6-oxo-2-phenylpyrimidin-1(6H)-yl)acetate

A vial was charged with tert-butyl 2-(5-bromo-6-oxo-2-phenyl-pyrimidin-1-yl)acetate (0.05 g, 0.1369 mmol), 4-(2-aminoethyl)benzonitrile (0.02289 g, 0.1369 mmol), cesium carbonate (0.0893 g, 0.2738 mmol), BINAP (0.01705 g, 0.02738 mmol) and palladium acetate (0.003074 g, 0.01369 mmol). Toluene was added to the reaction mixture, and the reaction mixture was purged with nitrogen for 3 minutes. The reaction mixture was allowed to stir at 120° C. for 10 minutes under microwave radiation. The crude mixture was purified using flash chromatography (silica gel, 0-50% EtOAc in heptane) to give tert-butyl 2-(5-((4-cyanophenethyl)amino)-6-oxo-2-phenylpyrimidin-1(6H)-yl)acetate (0.02 g, 30% yield). LC/MS (ESI) m/z: 431 (M+H)⁺.

### Step 2: 2-(5-((4-cyanophenethyl)amino)-6-oxo-2-phenylpyrimidin-1(6H)-yl)acetic acid

tert-butyl 2-(5-((4-cyanophenethyl)amino)-6-oxo-2-phenylpyrimidin-1(6H)-yl)acetate (0.02 g, 0.044 mmol) was dissolved DCM (3 mL). TFA (3 ML) was added in it and reaction was allowed to stir at rt for 1 hr. Excess TFA was evaporated, and 1 M HCl in MeOH was added into the crude mass, which was allowed to stir for 5 minutes. Methanol was evaporated to dryness to give 2-(5-((4-cyanophenethyl)amino)-6-oxo-2-phenylpyrimidin-1(6H)-yl)acetic acid (0.015 g, 98% yield) as a white solid which was used in the next step without further purification. LC/MS (ESI) m/z: 375 (M+H)⁺.

### Step 3: N-((1H-pyrrolo[3,2-c]pyridine-2-yl)methyl)-2-(5-((4-cyanophenethyl)amino)-6-oxo-2-phenylpyrimidin-1(6H)-yl)acetamide (Compound 28)

2-[5-[2-(4-cyanophenyl)ethylamino]-6-oxo-2-phenyl-pyrimidin-1-yl]acetic acid (0.05 g, 0.1 mmol), (1H-pyrrolo[3,2-c]pyridine-2-yl)methanamine hydrochloride (0.04 g, 0.3 mmol), TBTU (0.09 g, 0.3 mmol), and DIPEA (0.04 mL, 0.3 mmol) were stirred in DMF (5 mL) at rt for 30 minute. Water (30 mL)) was added into the reaction mixture and solid was filtered. Solid was dissolved in DCM and dried over anhydrous Na₂SO₄, filtered, and concentrated to dryness. The residue was purified by flash chromatography (silica gel, 0-5% MeOH in DCM) to give Compound 28 (7 mg, 10% yield) as a white solid. ¹H NMR (400 MHz, CDCl₃-MeOD) δ 8.71 (s, 1H), 8.13 (d, J=6.6 Hz, 1H), 7.64 (d, J=8.6 Hz, 2H), 7.55-7.37 (m, 7H), 7.34 (q, J=4.7 Hz, 2H), 7.19 (s, 1H), 6.46 (s, 1H), 4.59 (s, 2H), 4.55 (s, 2H), 3.52-3.43 (m, 2H), 3.11-3.02 (m, 2H). LC/MS (ESI) m/z: 504 (M+H)⁺. RT (Method A): 1.17 min.

### Scheme 43. Synthesis of N-((1H-pyrrolo[3,2-c]pyridine-2-yl)methyl)-2-(5-((3-(((3R,3aR,6R,6aR)-6-hydroxyhexahydrofuro[3,2-b]furan-3-yl)oxy)propyl)amino)-6-oxo-2-phenylpyrimidin-1(6H)-yl)acetamide (Compound 190)

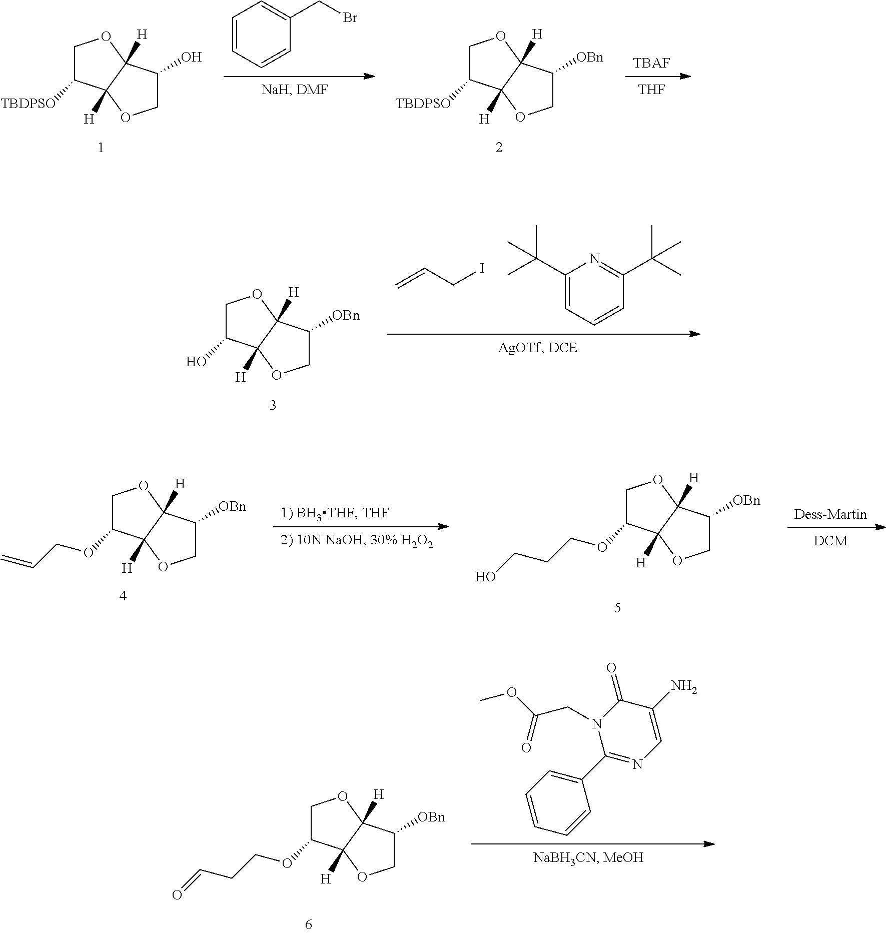

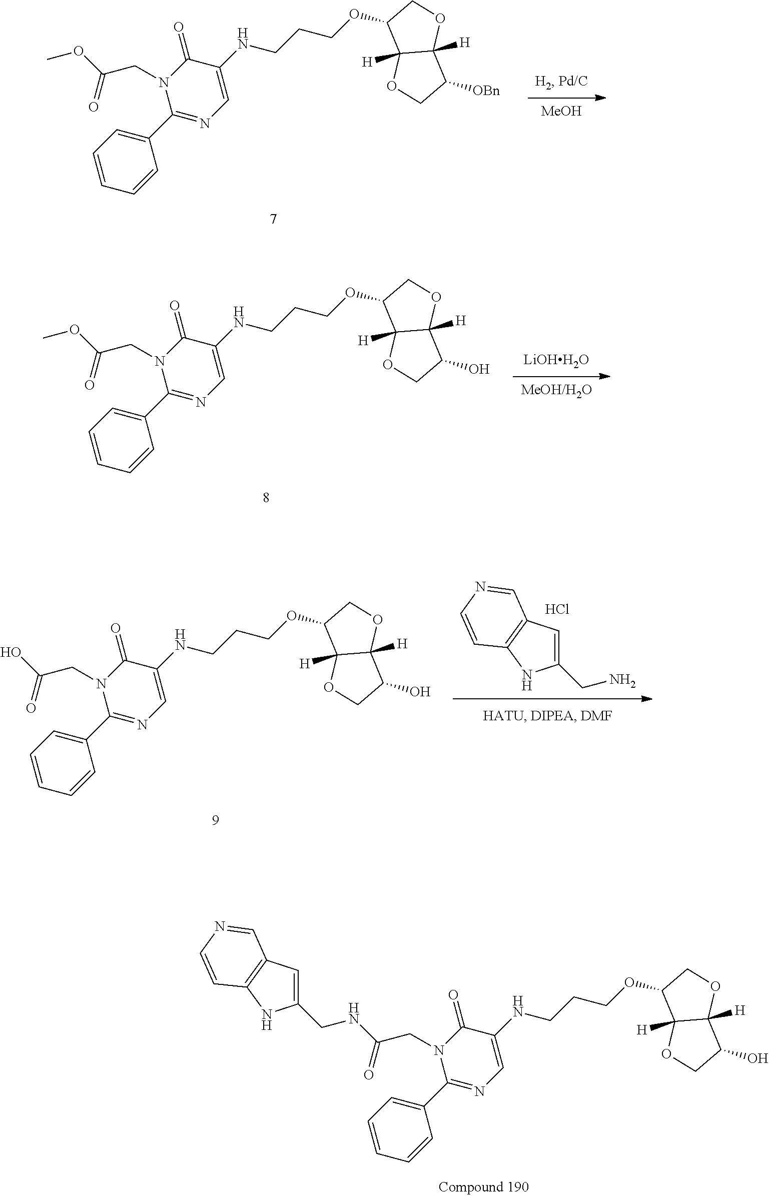

### Step 1: (((3R,3aS,6R,6aR)-6-(benzyloxy)hexahydrofuro[3,2-b]furan-3-yl)oxy)(tert-butyl)diphenylsilane (2)

To a solution of (3R,3aR,6R,6aS)-6-((tert-butyldiphenylsilyl)oxy)hexahydrofuro[3,2-b]furan-3-ol (5.0 g, 13.02 mmol) in DMF (50 mL) was added NaH (781 mg, 19.53 mmol, 60% dispersion in mineral oil) in portions at 0° C. and the mixture was stirred at 0° C. for 0.5 hour. And then, to the reaction mixture was added (bromomethyl)benzene (3.34 g, 19.53 mmol) drop-wisely at 0° C. and the reaction mixture was stirred at room temperature overnight. The mixture was quenched with saturated aq.NH₄Cl solution and extracted with EtOAc twice. The combined organic layers were washed with brine, dried over anhydrous Na₂SO₄, filtered, and concentrated to dryness. The residue was purified by flash chromatography on silica gel (0-10% EtOAc in PE) to give the title compound (4.23 g, yield 68.4%) as a colorless oil. LC/MS (ESI) m/z: 475 (M+H)⁺.

### Step 2: (3R,3aR,6R,6aR)-6-(benzyloxy)hexahydrofuro[3,2-b]furan-3-ol (3)

To a solution of (((3R,3aS,6R,6aR)-6-(benzyloxy)hexahydrofuro[3,2-b]furan-3-yl)oxy)(tert-butyl)diphenylsilane (4.2 g, 8.86 mmol) in THF (42 mL) was added TBAF (13 mL, 4M in THF) at 0° C. and the mixture was stirred at room temperature for 3 hours. The mixture was diluted with EtOAc, washed with water and brine, dried over anhydrous Na₂SO₄, filtered, and concentrated under reduced pressure to dryness. The residue was purified by flash chromatography on silica gel (0-7% MeOH in DCM) to give the title compound (1.32 g, yield 63.2%) as a colorless oil. LC/MS (ESI) m/z: 237 (M+H)⁺.

### Step 3: (3R,3aR,6R,6aR)-3-(allyloxy)-6-(benzyloxy)hexahydrofuro[3,2-b]furan (4)

To a solution of AgOTf (1.31 g, 5.08 mmol) in DCE (20 mL) was added a solution of (3R,3aR,6R,6aR)-6-(benzyloxy)hexahydrofuro[3,2-b]furan-3-ol (400 mg, 1.69 mmol) in DCE (20 mL), 2,6-di-tert-butylpyridine (1.30 g, 6.78 mmol) and 3-iodoprop-1-ene (1.14 g, 6.78 mmol) drop-wisely at 0° C. under N₂ atmosphere and the mixture was stirred at 30° C. under N₂ atmosphere overnight. The reaction mixture was diluted with DCM and filtered. The filtrate was washed with water and brine, dried over anhydrous Na₂SO₄, filtered, and concentrated under reduced pressure to dryness. The residue was purified by flash chromatography on silica gel (0-20% EtOAc in PE) to give the title compound (300 mg, yield 64.1%) as a colorless oil. LC/MS (ESI) m/z: 277 (M+H)⁺.

### Step 4: 3-(((3R,3aR,6R,6aR)-6-(benzyloxy)hexahydrofuro[3,2-b]furan-3-yl)oxy) propan-1-ol (5)

To a solution of (3R,3aR,6R,6aR)-3-(allyloxy)-6-(benzyloxy)hexahydrofuro[3,2-b]furan (150 mg, 0.54 mmol) in THF (2 mL) was added BH₃·THF (1.0 mL, 1M in THF) drop-wisely at 0° C. and the mixture was stirred at room temperature for 2.5 hours. The above mixture was cooled downed 0° C. and to the reaction mixture was added aq.NaOH solution (1.0 mL, 10M) and H₂O₂ (1.0 mL, 30% wt.) successively at 0° C. The resulting mixture was stirred at room temperature for 1 hour. The mixture was diluted with EtOAc, washed with water and brine, dried over anhydrous Na₂SO₄, filtered, and concentrated under reduced pressure to dryness. The residue was purified by flash chromatography on silica gel (0-7% MeOH in DCM) to give the title compound (70 mg, yield 44.0%) as a colorless oil. LC/MS (ESI) m/z: 295 (M+H)⁺.

### Step 5: 3-(((3R,3aR,6R,6aR)-6-(benzyloxy)hexahydrofuro[3,2-b]furan-3-yl)oxy) propanal (6)

To a solution of 3-(((3R,3aR,6R,6aR)-6-(benzyloxy)hexahydrofuro[3,2-b]furan-3-yl)oxy)propan-1-ol (70 mg, 0.24 mmol) in DCM (2 mL) was added Dess-Martin periodinane (303 mg, 0.71 mmol) at 0° C. and the reaction mixture was stirred at room temperature for 2 hours. The mixture was diluted with DCM, washed with saturated aq.Na₂S₂O₃ solution and brine, dried over anhydrous Na₂SO₄, filtered, and concentrated under reduced pressure to dryness. The residue was purified by flash chromatography (silica gel, 0-5% MeOH in DCM) to give the title compound (60 mg, yield 86.9%) as a white solid. LC/MS (ESI) m/z: 293 (M+H)⁺.

### Step 6: Methyl 2-(5-((3-(((3R,3aR,6R,6aR)-6-(benzyloxy)hexahydrofuro[3,2-b]furan-3-yl)oxy)propyl)amino)-6-oxo-2-phenylpyrimidin-1(6H)-yl)acetate (7)

To a mixture of methyl 3-(((3R,3aR,6R,6aR)-6-(benzyloxy)hexahydrofuro[3,2-b]furan-3-yl)oxy)propanal (30 mg, 0.10 mmol) and methyl 2-(5-amino-6-oxo-2-phenylpyrimidin-1(6H)-yl)acetate (26 mg, 0.10 mmol) in MeOH (2 mL) was added NaBH₃CN (19 mg, 0.30 mmol) at room temperature and the reaction mixture was stirred at 50° C. for 2 hours. The mixture was concentrated under reduced pressure to dryness. The residue was purified by prep-HPLC to give the title compound (20 mg, yield 37.3%) as a white solid. LC/MS (ESI) m/z: 536 (M+H)⁺.

### Step 7: Methyl 2-(5-((3-(((3R,3aR,6R,6aR)-6-hydroxyhexahydrofuro[3,2-b]furan-3-yl)oxy)propyl)amino)-6-oxo-2-phenylpyrimidin-1(6H)-yl)acetate (8)

To a solution of methyl 2-(5-((3-(((3R,3aR,6R,6aR)-6-(benzyloxy)hexahydrofuro[3,2-b]furan-3-yl)oxy)propyl)amino)-6-oxo-2-phenylpyrimidin-1(6H)-yl)acetate (20 mg, 0.037 mmol) in MeOH (1 mL) was added Pd(OH)₂ (20 mg, 10% wt.) and three drops AcOH, the mixture was degassed under N₂ atmosphere for three times and stirred under a H₂ balloon at room temperature for 2 days. The mixture was filtered, and the filtrate was concentrated to dryness to give the title compound (15 mg, yield 90.9%) as a colorless oil, which was used directly in the next step without further purification. LC/MS (ESI) m/z: 446 (M+H)⁺.

### Step 8: 2-(5-((3-(((3R,3aR,6R,6aR)-6-hydroxyhexahydrofuro[3,2-b]furan-3-yl)oxy) propyl)amino)-6-oxo-2-phenylpyrimidin-1(6H)-yl)acetic acid (9)

To a solution of methyl 2-(5-((3-(((3R,3aR,6R,6aR)-6-hydroxyhexahydrofuro[3,2-b]furan-3-yl)oxy)propyl)amino)-6-oxo-2-phenylpyrimidin-1(6H)-yl)acetate (15 mg, 0.034 mmol) in MeOH/H₂O (1 mL, 4/1) was added LiOH·H₂O (4.0 mg, 0.10 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (10 mg, yield 68.5%) as a white solid, which was used directly in the next step without further purification. LC/MS (ESI) m/z: 432 (M+H)⁺.

### Step 9: N-((1H-pyrrolo[3,2-c]pyridine-2-yl)methyl)-2-(5-((3-(((3R,3aR,6R,6aR)-6-hydroxyhexahydrofuro[3,2-b]furan-3-yl)oxy)propyl)amino)-6-oxo-2-phenylpyrimidin-1(6H)-yl)acetamide (Compound 190)

To a mixture of 2-(5-((3-(((3R,3aR,6R,6aR)-6-hydroxyhexahydrofuro[3,2-b]furan-3-yl)oxy)propyl)amino)-6-oxo-2-phenylpyrimidin-1(6H)-yl)acetic acid (10 mg, 0.023 mmol) and (1H-pyrrolo[3,2-c]pyridine-2-yl)methanamine hydrochloride (8 mg, 0.042 mmol) in DMF (1 mL) was added DIPEA (15 mg, 0.12 mmol) and HATU (13 mg, 0.035 mmol) under N₂ atmosphere and the reaction mixture was stirred at room temperature for 2 hours. The mixture was concentrated under reduced pressure to dryness. The residue was purified by prep-HPLC to give Compound 190 (1.0 mg, yield 7.8%) as a yellow solid. ¹H-NMR (400 MHz, CD₃OD) δ 8.70 (s, 1H), 8.11 (d, J=6.6 Hz, 1H), 7.47 (d, J=8.0 Hz, 3H), 7.40 (d, J=6.9 Hz, 3H), 7.17 (s, 1H), 6.47 (s, 1H), 4.62 (s, 2H), 4.54 (s, 2H), 4.43 (d, J=4.9 Hz, 1H), 4.24 (d, J=6.9 Hz, 1H), 4.10-4.06 (m, 1H), 4.03 (d, J=8.3 Hz, 1H), 3.93 (d, J=1.9 Hz, 1H), 3.81-3.76 (m, 1H), 3.68 (s, 1H), 3.58 (s, 1H), 2.89 (dd, J=16.6, 6.9 Hz, 2H), 2.24-2.13 (m, 1H), 2.03 (d, J=6.5 Hz, 1H), 1.98-1.91 (m, 2H). LC/MS (ESI) m/z: 561 (M+H)⁺. RT (Method A): 0.46 min.

### Scheme 44. Synthesis of N-((1H-pyrrolo[3,2-c]pyridine-2-yl)methyl)-2-(5-((3-(4-(((3R,3aR,6R,6aR)-6-methoxyhexahydrofuro[3,2-b]furan-3-yl)oxy)phenyl)propyl) amino)-6-oxo-2-phenylpyrimidin-1(6H)-yl)acetamide (Compound 192)

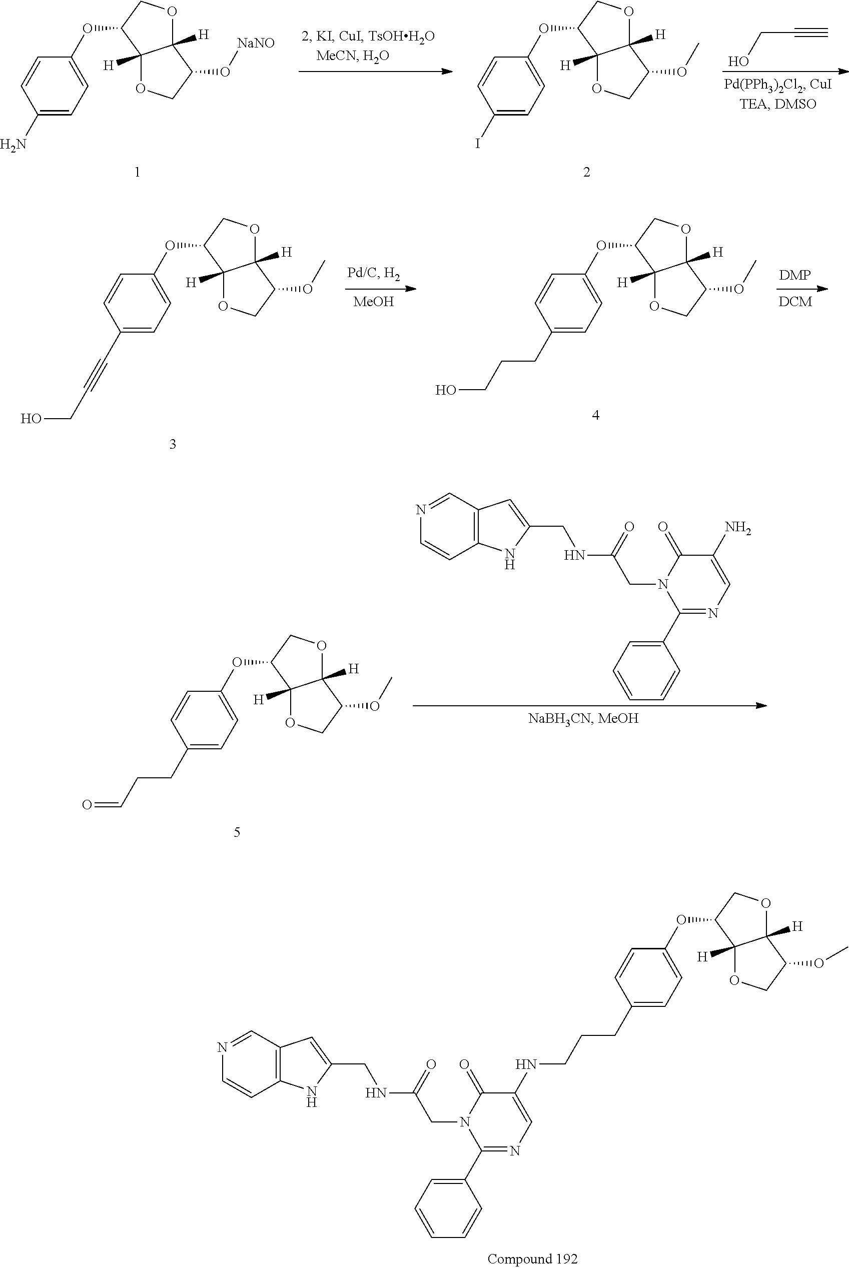

### Step 1: (3R,3aR,6R,6aR)-3-(4-iodophenoxy)-6-methoxyhexahydrofuro[3,2-b]furan (2)

To a solution of TsOH·H₂O (227 mg, 1.2 mmol) in MeCN (4 mL) was added 4-(((3R,3aR,6R,6aR)-6-methoxyhexahydrofuro[3,2-b]furan-3-yl)oxy)aniline (150 mg, 0.6 mmol), NaNO₂ (124 mg, 1.8 mmol) and KI (248 mg, 1.5 mmol) at 0° C. The above mixture was added CuI (285 mg, 1.5 mmol) at 0° C. for 0.5 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (135 mg, yield 62.5%) as a yellow solid. LC/MS (ESI) m/z: 363 (M+H)⁺.

### Step 2: 3-(4-(((3R,3aR,6R,6aR)-6-methoxyhexahydrofuro[3,2-b]furan-3-yl)oxy) phenyl)prop-2-yn-1-ol (3)

To a mixture of (3R,3aR,6R,6aR)-3-(4-iodophenoxy)-6-methoxyhexahydrofuro[3,2-b]furan (130 mg, 0.36 mmol) and prop-2-yn-1-ol (29 mg, 0.54 mmol) in TEA (1 mL)/DMSO (1 mL) was added CuI (3 mg, 0.02 mmol) and Pd(PPh₃)₂Cl₂ (13 mg, 0.02 mmol) under N₂ atmosphere, the mixture was degassed under N₂ atmosphere for three times and stirred at room temperature for 3 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give the title compound (94 mg, yield 90.4%) as a yellow solid. LC/MS (ESI) m/z: 291 (M+H)⁺.

### Step 3: 3-(4-(((3R,3aR,6R,6aR)-6-methoxyhexahydrofuro[3,2-b]furan-3-yl)oxy) phenyl)propan-1-ol (4)

To a solution of 3-(4-(((3R,3aR,6R,6aR)-6-methoxyhexahydrofuro[3,2-b]furan-3-yl)oxy)phenyl)prop-2-yn-1-ol (90 mg, 0.31 mmol) in MeOH (3 mL) was added Pd/C (10 mg, 10% wt.), the mixture was degassed under N₂ atmosphere for three times and stirred under a H₂ balloon at room temperature overnight. The mixture was filtered, and the filtrate was concentrated to dryness to give 3-(4-(((3R,3aR,6R,6aR)-6-methoxyhexahydrofuro[3,2-b]furan-3-yl)oxy)phenyl)propan-1-ol (85 mg, yield 93.4%) as a colorless oil, which was used directly in the next step without further purification. LC/MS (ESI) m/z: 257 (M+H)⁺.

### Step 4: 3-(4-(((3R,3aR,6R,6aR)-6-methoxyhexahydrofuro[3,2-b]furan-3-yl)oxy) phenyl)propanal (5)

To a solution of 3-(4-(((3R,3aR,6R,6aR)-6-methoxyhexahydrofuro[3,2-b]furan-3-yl)oxy)phenyl)propan-1-ol (80 mg, 0.27 mmol) in DCM (3 mL) was added DMP (173 mg, 0.41 mmol) at 0° C. and the mixture was stirred at room temperature for 2 hours. The mixture was quenched with aq.Na₂S₂O₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (40 mg, yield 50.3%) as a colorless oil.

### Step 5: N-((1H-pyrrolo[3,2-c]pyridine-2-yl)methyl)-2-(5-((3-(4-(((3R,3aR,6R,6aR)-6-methoxyhexahydrofuro[3,2-b]furan-3-yl)oxy)phenyl)propyl)amino)-6-oxo-2-phenylpyrimidin-1(6H)-yl)acetamide (Compound 192)

To a mixture of N-((1H-pyrrolo[3,2-c]pyridine-2-yl)methyl)-2-(5-amino-6-oxo-2-phenylpyrimidin-1(6H)-yl)acetamide (30 mg, 0.08 mmol) and 3-(4-(((3R,3aR,6R,6aR)-6-methoxyhexahydrofuro[3,2-b]furan-3-yl)oxy)phenyl)propanal (20 mg, 0.08 mmol) in MeOH (3 mL) was added NaBH₃CN (21 mg, 0.32 mmol) and MgSO₄ (30 mg) under N₂ atmosphere and the reaction mixture was stirred at 50° C. for 16 hours. The mixture was filtered and concentrated under reduced pressure to dryness. The residue was purified by prep-HPLC to give Compound 192 (1.0 mg, yield 1.9%) as a white solid. ¹H-NMR (400 MHz, CD₃OD): δ 8.84 (s, 1H), 8.18 (d, J=6.1 Hz, 1H), 7.59 (d, J=6.1 Hz, 1H), 7.48 (t, J=6.4 Hz, 3H), 7.43-7.38 (m, 2H), 7.14 (d, J=8.6 Hz, 2H), 7.08 (s, 1H), 6.92 (d, J=8.6 Hz, 2H), 6.65 (s, 1H), 4.81 (d, J=4.4 Hz, 2H), 4.63 (s, 2H), 4.58 (s, 1H), 4.13-4.08 (m, 1H), 4.01-3.93 (m, 2H), 3.86 (m, J=9.2, 6.1 Hz, 1H), 3.63 (t, J=6.8 Hz, 1H), 3.45 (s, 3H), 3.17 (t, J=7.0 Hz, 2H), 2.69 (t, J=7.5 Hz, 2H), 1.99-1.92 (m, 2H). LC/MS (ESI) m/z: 651 (M+H)⁺. RT (Method A): 1.17 min.

### Scheme 45. Synthesis of N-((1H-pyrrolo[3,2-c]pyridine-2-yl)methyl)-2-(2-(4-(((3R,3aR,6R,6aR)-6-methoxyhexahydrofuro[3,2-b]furan-3-yl)oxy)phenyl)-6-oxo-5-((3-phenylpropyl)amino)pyrimidin-1(6H)-yl)acetamide (Compound 189)

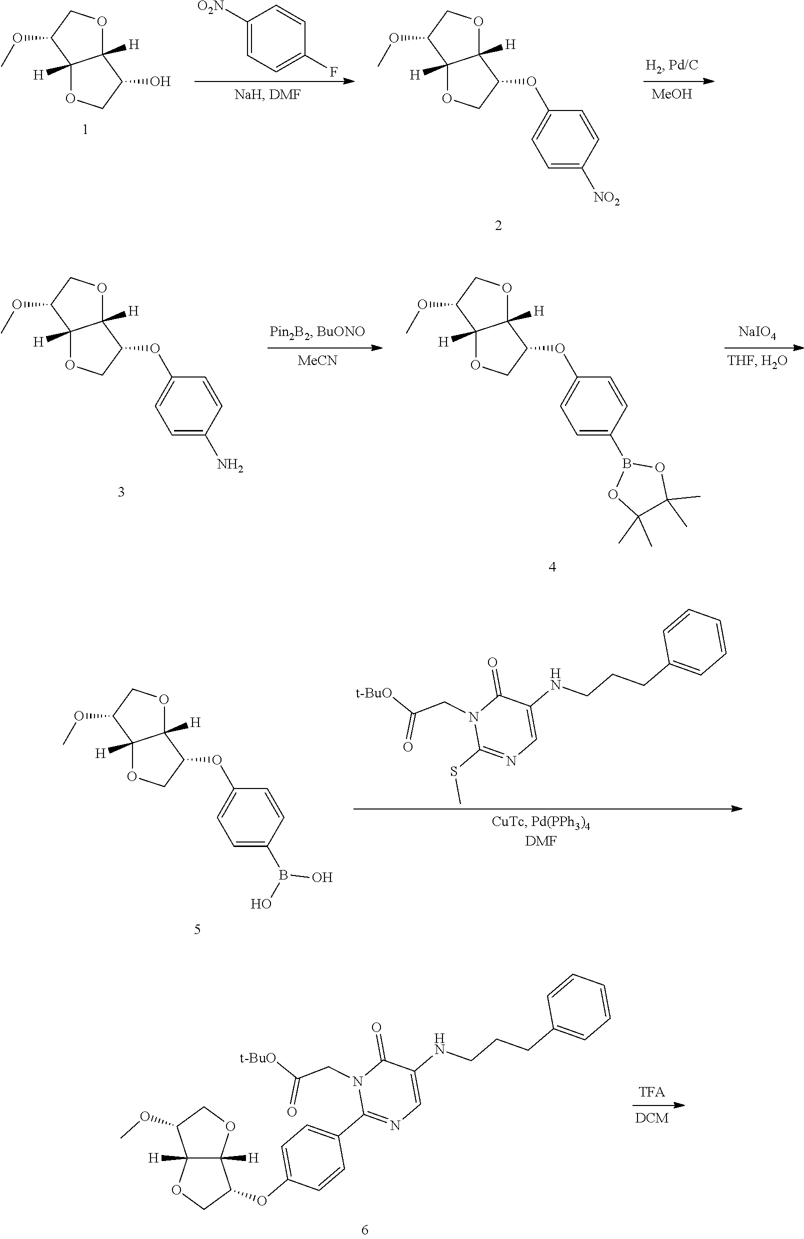

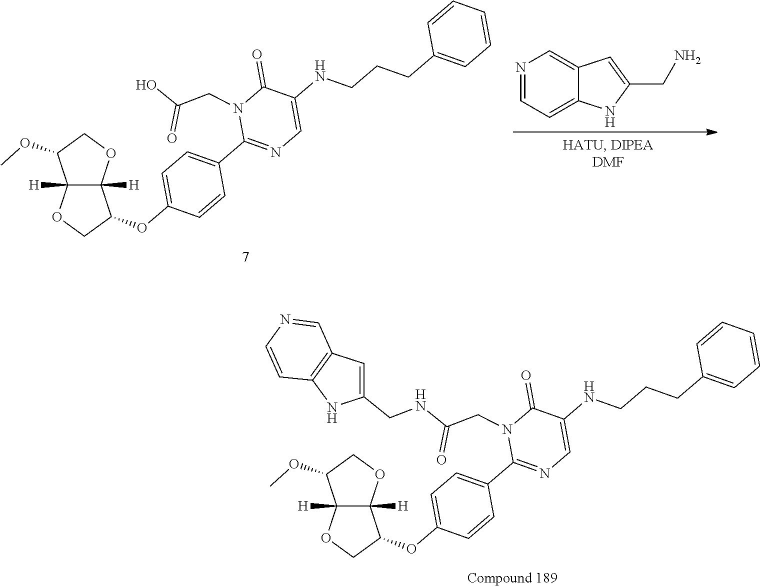

### Step 1: (3R,3aR,6R,6aR)-3-methoxy-6-(4-nitrophenoxy)hexahydrofuro[3,2-b]furan (2)

To a solution of (3R,3aR,6R,6aR)-6-methoxyhexahydrofuro[3,2-b]furan-3-ol (600 mg, 3.7 mmol) in DMF (3 mL) was added NaH (224 mg, 9.3 mmol) at 0° C. and the reaction solution was stirred at 0° C. for 0.5 hours. The mixture was added 1-fluoro-4-nitrobenzene (788 mg, 5.6 mmol) at 0° C. and the resulting mixture was stirred at room temperature for 3 hours. The mixture was diluted with aq.NH₄Cl and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (950 mg, yield 44.4%) as a yellow solid. LC/MS (ESI) m/z: 282 (M+H)⁺.

### Step 2: 4-(((3R,3aR,6R,6aR)-6-methoxyhexahydrofuro[3,2-b]furan-3-yl)oxy)aniline (3)

To a solution of (3R,3aR,6R,6aR)-3-methoxy-6-(4-nitrophenoxy)hexahydrofuro[3,2-b]furan (900 mg, 3.2 mmol) in MeOH (5 mL) was added Pd/C (90 mg, 10% wt.), the mixture was degassed under N₂ atmosphere for three times and stirred under a H₂ balloon at room temperature for 3 hours. The mixture was filtered, and the filtrate was concentrated to dryness to the title compound (790 mg, yield 98.4%) as a brown solid, which was used directly in the next step without further purification. LC/MS (ESI) m/z: 252 (M+H)⁺.

### Step 3: 2-(4-(((3R,3aR,6R,6aR)-6-methoxyhexahydrofuro[3,2-b]furan-3-yl)oxy) phenyl)-4,4,5,5-tetramethyl-1,3,2-dioxaborolane (4)

To a solution of 4-(((3R,3aR,6R,6aR)-6-methoxyhexahydrofuro[3,2-b]furan-3-yl)oxy)aniline (300 mg, 2.4 mmol) in MeCN (5 mL) was added BuONO (370 mg, 3.6 mmol) and Pin₂B₂ (899 mg, 5.91 mmol) at 0° C., the reaction mixture was stirred at 0° C. room temperature for 3 hours. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) and further purified by prep-HPLC to give the title compound (430 mg, yield 99.4%) as a yellow oil. LC/MS (ESI) m/z: 363 (M+H)⁺.

### Step 4: (4-(((3R,3aR,6R,6aR)-6-methoxyhexahydrofuro[3,2-b]furan-3-yl)oxy)phenyl) boronic acid (5)

To a solution of 2-(4-(((3R,3aR,6R,6aR)-6-methoxyhexahydrofuro[3,2-b]furan-3-yl)oxy)phenyl)-4,4,5,5-tetramethyl-1,3,2-dioxaborolane (100 mg, 0.3 mmol) in THF (2 mL) and H₂O (2 mL) was added NaIO₄ (236 mg, 1.1 mmol) under N₂ atmosphere and the reaction mixture was stirred at room temperature for 16 hours. The mixture was filtered and concentrated under reduced pressure to dryness. The residue was purified by prep-TLC (PE:EtOAc=1:1) to give the title compound (40 mg, yield 51.5%) as a colorless oil. LC/MS (ESI) m/z: 281 (M+H)⁺.

### Step 5: Tert-butyl 2-(2-(4-(((3R,3aR,6R,6aR)-6-methoxyhexahydrofuro[3,2-b]furan-3-yl)oxy)phenyl)-6-oxo-5-((3-phenylpropyl)amino)pyrimidin-1(6H)-yl)acetate (6)

To a mixture of (4-(((3R,3aR,6R,6aR)-6-methoxyhexahydrofuro[3,2-b]furan-3-yl)oxy)phenyl)boronic acid (40 mg, 0.14 mmol) and tert-butyl 2-(2-(methylthio)-6-oxo-5-((3-phenylpropyl)amino)pyrimidin-1(6H)-yl)acetate (56 mg, 0.14 mmol) in DMF (1 mL) was added Cu Tc (60 mg, 0.31 mmol) and Pd(PPh₃)₄ (17 mg, 0.01 mmol) at room temperature under N₂ atmosphere and the reaction mixture was degassed under N₂ atmosphere for three times and stirred at 70° C. overnight. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (PE:EtOAc=1:1) to give the title compound (30 mg, yield 36.4%) as a yellow oil. LC/MS (ESI) m/z: 578 (M+H)⁺.

### Step 6: 2-(2-(4-(((3R,3aR,6R,6aR)-6-methoxyhexahydrofuro[3,2-b]furan-3-yl)oxy) phenyl)-6-oxo-5-((3-phenylpropyl)amino)pyrimidin-1(6H)-yl)acetic acid (7)

A solution of tert-butyl 2-(2-(4-(((3R,3aR,6R,6aR)-6-methoxyhexahydrofuro[3,2-b]furan-3-yl)oxy)phenyl)-6-oxo-5-((3-phenylpropyl)amino)pyrimidin-1(6H)-yl)acetate (30 mg, 0.05 mmol) in DCM (1 mL)/TFA (1 mL) was stirred under N₂ atmosphere at room temperature for 2 hours. The reaction mixture was concentrated to dryness under reduced pressure to give the title compound (20 mg, yield 74.1%) as a yellow solid, which was used directly in the next step without further purification. LC/MS (ESI) m/z: 522 (M+H)⁺.

### Step 7: N-((1H-pyrrolo[3,2-c]pyridine-2-yl)methyl)-2-(2-(4-(((3R,3aR,6R,6aR)-6-methoxyhexahydrofuro[3,2-b]furan-3-yl)oxy)phenyl)-6-oxo-5-((3-phenylpropyl) amino)pyrimidin-1(6H)-yl)acetamide (Compound 189)

To a mixture of 2-(2-(4-(((3R,3aR,6R,6aR)-6-methoxyhexahydrofuro[3,2-b]furan-3-yl)oxy)phenyl)-6-oxo-5-((3-phenylpropyl)amino)pyrimidin-1(6H)-yl)acetic acid (15 mg, 0.03 mmol) and (1H-pyrrolo[3,2-c]pyridine-2-yl)methanamine (6 mg, 0.04 mmol) in DMF (2 mL) was added DIPEA (19 mg, 0.14 mmol) and HATU (16 mg, 0.04 mmol) under N₂ atmosphere and the reaction mixture was stirred at room temperature for 2 hours. The mixture was filtered and concentrated under reduced pressure to dryness. The residue was purified by prep-HPLC to give Compound 189 (3 mg, yield 16.0%) as a yellow solid. ¹H-NMR (400 MHz, CD₃OD): δ 8.94 (s, 1H), 8.24 (d, J=6.4 Hz, 1H), 7.73 (d, J=6.4 Hz, 1H), 7.40 (d, J=8.7 Hz, 2H), 7.29-7.21 (m, 4H), 7.17 (d, J=7.0 Hz, 1H), 7.07 (s, 1H), 6.99 (d, J=8.7 Hz, 2H), 6.74 (s, 1H), 4.69-4.63 (m, 3H), 4.60 (s, 1H), 4.11-4.07 (m, 1H), 4.00-3.88 (m, 3H), 3.59 (t, J=8.4 Hz, 1H), 3.47 (s, 3H), 3.17 (t, J=7.0 Hz, 2H), 2.74 (t, J=7.6 Hz, 2H), 2.02-1.95 (m, 2H). LC/MS (ESI) m/z: 651 (M+H)⁺. RT (Method A): 1.32 min.

### Scheme 46. Synthesis of N-((1H-pyrrolo[3,2-c]pyridine-2-yl)methyl)-2-(5-((3-(4-(3-fluorooxetan-3-yl)phenyl)propyl)amino)-6-oxo-2-phenylpyrimidin-1(6H)-yl)acetamide (Compound 116)

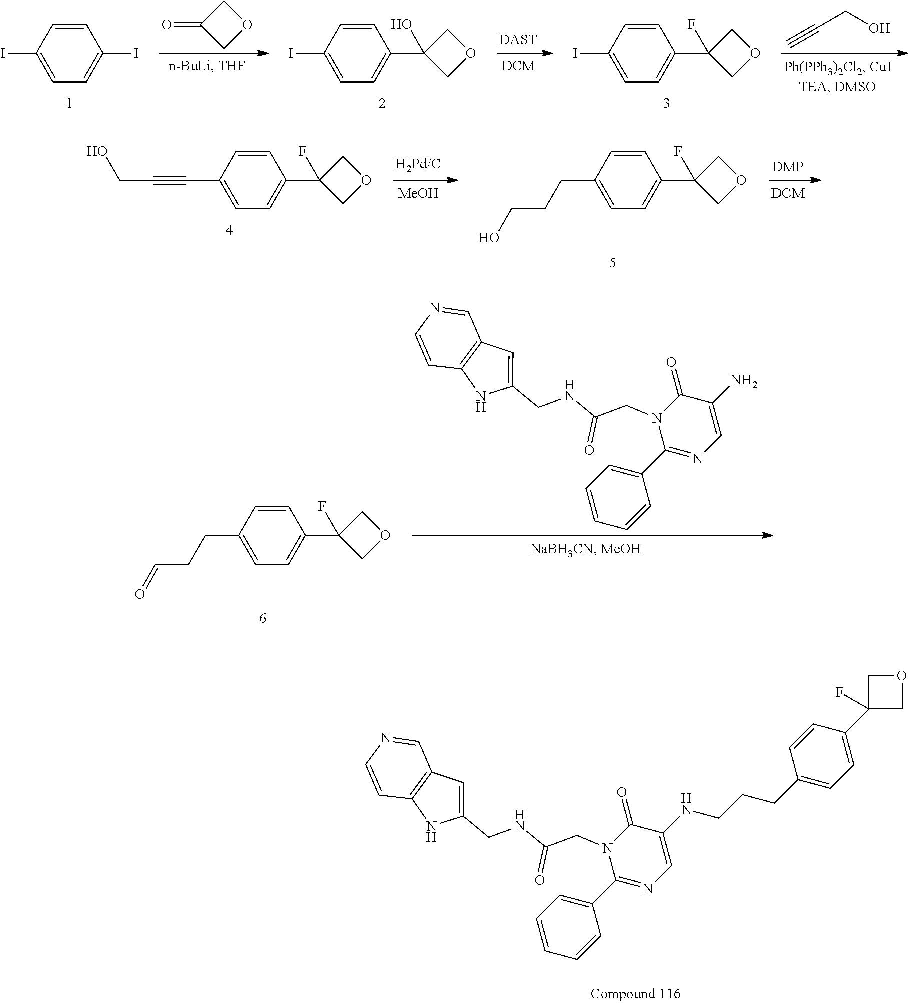

### Step 1: 3-(4-Iodophenyl)oxetan-3-ol (2)

To a mixture of 1,4-diiodobenzene (4.6 g, 13.9 mmol) and oxetan-3-one (1 g, 13.9 mmol) in THF (15 mL) was added n-BuLi (6.1 mL, 15.3 mmol, 2.5 M in THF) drop-wisely under N₂ atmosphere and the reaction mixture was stirred at −65° C. for 2 hours. The mixture was quenched with 1 N aq. HCl solution and extracted with EtOAc twice. The combined organic layers were dried over anhydrous Na₂SO₄, filtered, and concentrated under reduced pressure to dryness. The residue was purified by flash chromatography (silica gel, 0-5% EtOAc in PE) to give the title compound (1.1 g, yield 28.8%) as a yellow oil.

### Step 2: 3-Fluoro-3-(4-iodophenyl)oxetane (3)

To a solution of 3-(4-iodophenyl)oxetan-3-ol (1.1 g, 4.0 mmol) in DCM (15 mL) was added BAST (1.7 g, 10.8 mmol) drop-wisely at 0° C. under N₂ atmosphere and the reaction mixture was stirred at room temperature for 4 hours. The mixture was quenched with saturated aq.NaHCO₃ solution and extracted with EtOAc twice. The combined organic layers were dried over anhydrous Na₂SO₄, filtered, and concentrated under reduced pressure to dryness. The residue was purified by flash chromatography (silica gel, 0-5% EtOAc in PE) to give 3-fluoro-3-(4-iodophenyl)oxetane (600 mg, yield 61.1%) as a colorless oil. ¹H NMR (400 MHz, CDCl₃) δ 7.77 (d, J=8.2 Hz, 2H), 7.31 (d, J=8.4 Hz, 2H), 5.12 (d, J=8.7 Hz, 1H), 5.07 (d, J=8.7 Hz, 1H), 4.83 (d, J=8.8 Hz, 1H), 4.78 (d, J=8.8 Hz, 1H).

### Step 3: 3-(4-(3-Fluorooxetan-3-yl)phenyl)prop-2-yn-1-ol (4)

To a solution of 3-fluoro-3-(4-iodophenyl)oxetane (800 mg, 2.9 mmol) in TEA (5 mL) and DMSO (5 mL) was added prop-2-yn-1-ol (320 mg, 5.8 mmol), Pd(PPh₃)₂Cl₂ (200 mg, 0.29 mmol) and CuI (56 mg, 0.29 mmol) under N₂ atmosphere, the reaction mixture was degassed under N₂ atmosphere for three times and stirred at room temperature for 10 minutes. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give the title compound (350 mg, yield 58.6%) as a colorless oil. ¹H NMR (400 MHz, CDCl₃) δ 7.45 (d, J=1.5 Hz, 4H), 5.23 (s, 1H), 5.06 (d, J=8.8 Hz, 1H), 5.01 (d, J=8.8 Hz, 1H), 4.79 (s, 1H), 4.76 (s, 1H), 4.44 (s, 2H).

### Step 4: 3-(4-(3-Fluorooxetan-3-yl)phenyl)propan-1-ol (5)

To a solution of 3-(4-(3-fluorooxetan-3-yl)phenyl)prop-2-yn-1-ol (350 mg, 1.7 mmol) in MeOH (5 mL) was added Pd/C (40 mg, 10% wt.), the mixture was degassed under N₂ atmosphere for three times and stirred under a H₂ balloon at 25° C. overnight. The mixture was filtered, and the filtrate was concentrated to dryness to give the title compound (300 mg, yield 84.0%) as a light-yellow oil, which was used directly in the next step without further purification. ¹H NMR (400 MHz, CDCl₃) δ 7.41 (dd, J=13.8, 11.4 Hz, 2H), 7.21 (s, 2H), 5.07-4.99 (m, 2H), 4.85-4.77 (m, 2H), 4.37 (d, J=6.4 Hz, 1H), 3.62 (dd, J=7.8, 4.8 Hz, 1H), 2.67 (t, J=7.6 Hz, 1H), 1.87-1.81 (m, 1H), 1.22 (dd, J=17.2, 14.0 Hz, 2H).

### Step 5: 3-(4-(3-Fluorooxetan-3-yl)phenyl)propanal (6)

To a solution of 3-(4-(3-fluorooxetan-3-yl)phenyl)propan-1-ol (50 mg, 0.48 mmol) in DCM (1 mL) was added Dess-Martin periodinane (100 mg, 0.48 mmol) under N₂ atmosphere and the reaction mixture was stirred at room temperature for 5 minutes. The mixture was quenched with saturated aq.NaHSO₃ solution and extracted with DCM twice. The combined organic layer was dried over anhydrous Na₂SO₄, filtered, and concentrated under reduced pressure to dryness to give the title compound (25 mg, yield 25%) as a colorless oil, which was used directly in the next step without further purification.

### Step 6: N-((1H-pyrrolo[3,2-c]pyridine-2-yl)methyl)-2-(5-((3-(4-(3-fluorooxetan-3-yl)phenyl)propyl)amino)-6-oxo-2-phenylpyrimidin-1(6H)-yl)acetamide (Compound 116)

To a mixture of 3-(4-(3-fluorooxetan-3-yl)phenyl)propanal (25 mg, 0.12 mmol) and N-((1H-pyrrolo[3,2-c]pyridine-2-yl)methyl)-2-(5-amino-6-oxo-2-phenylpyrimidin-1(6H)-yl)acetamide (10 mg, 0.027 mmol) in MeOH (0.6 mL) was added NaBH₃CN (5 mg, 0.06 mmol) under N₂ atmosphere and the reaction mixture was stirred at 50° C. for 10 minutes. The mixture was purified by prep-HPLC to give Compound 116 (1.7 mg, yield 11.1%) as a white solid. ¹H NMR (400 MHz, CD₃OD) δ 8.70 (d, J=0.9 Hz, 1H), 8.10 (d, J=5.8 Hz, 1H), 7.49-7.45 (m, 5H), 7.39 (dd, J=6.4, 2.8 Hz, 3H), 7.34 (d, J=8.0 Hz, 2H), 7.08 (s, 1H), 6.47 (s, 1H), 5.07-4.91 (m, 4H), 4.62 (s, 2H), 4.54 (s, 2H), 3.19 (t, J=6.9 Hz, 2H), 2.79 (t, J=7.6 Hz, 2H), 2.03-1.99 (m, 2H). LC/MS (ESI) m/z: 567 (M+H)⁺. RT (Method A): 1.40 min.

### Scheme 47. Synthesis of N-((1H-pyrrolo[3,2-c]pyridine-2-yl)methyl)-2-(2-(4-(S-methylsulfonimidoyl)phenyl)-6-oxo-5-((3-phenylpropyl)amino)pyrimidin-1(6H)-yl)acetamide (Compound 200)

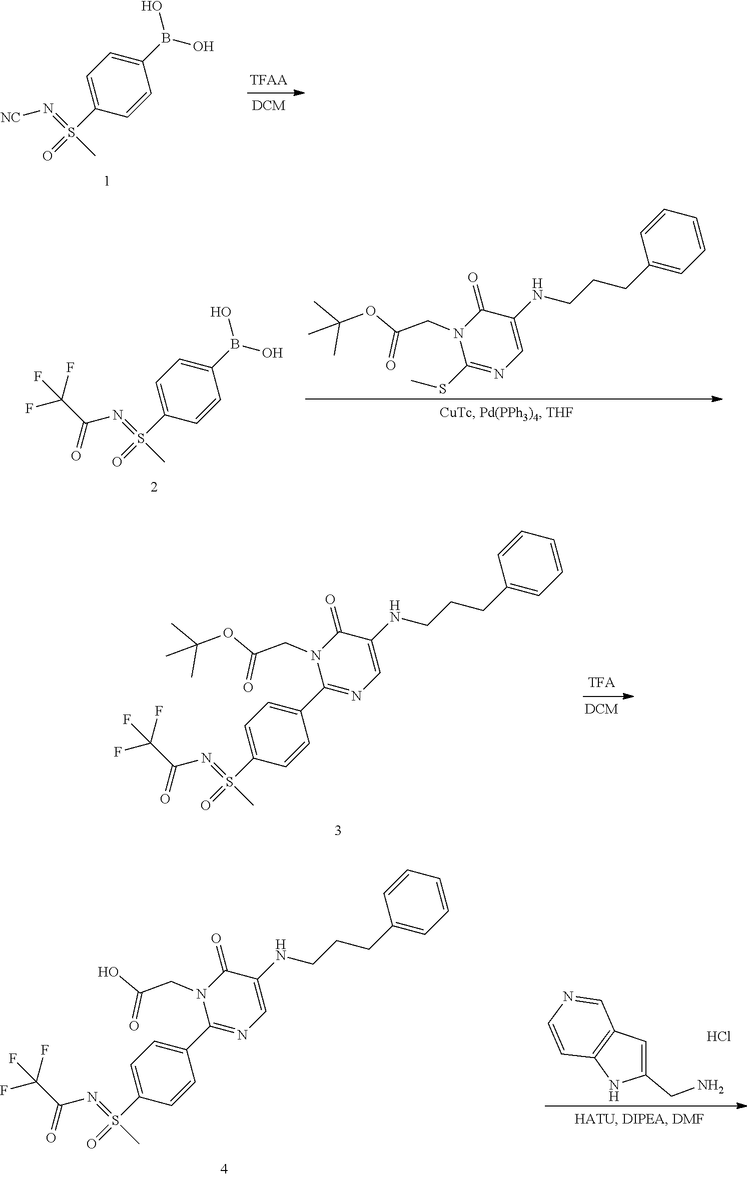

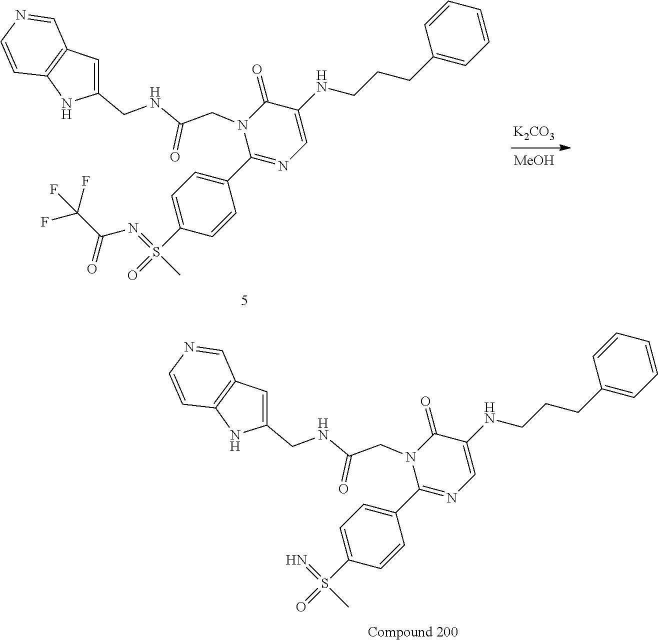 [SMILES: "MeOH" ; CS(=O)(=NC(=O)C(F)(F)F)c1ccc(-c2ncc(NCCCc3ccccc3)c(=O)n2CC(=O)NCc2cc3cnccc3[nH]2)cc1 ; "K2CO3" ; CS(=N)(=O)c1ccc(-c2ncc(NCCCc3ccccc3)c(=O)n2CC(=O)NCc2cc3cnccc3[nH]2)cc1]

### Step 1: (4-(S-methyl-N-(2,2,2-trifluoroacetyl)sulfonimidoyl)phenyl)boronic acid (2)

To a solution of (4-(N-cyano-S-methylsulfonimidoyl)phenyl)boronic acid (500 mg, 2.26 mmol) in DCM (5 mL) was added TFAA (950 mg, 4.52 mmol) drop-wisely at 0° C. and the mixture was stirred at room temperature for 6 hours. The reaction mixture was concentrated to dryness. The residue was purified by flash chromatography (silica gel, 0-10% MeOH in DCM) to give the title compound (250 mg, yield 37.4%) as a white solid. LC/MS (ESI) m/z: 296 (M+H)⁺.

### Step 2: Tert-butyl 2-(2-(4-(S-methyl-N-(2,2,2-trifluoroacetyl)sulfonimidoyl)phenyl)-6-oxo-5-((3-phenylpropyl)amino)pyrimidin-1(6H)-yl)acetate (3)

To a mixture of tert-butyl 2-(2-(methylthio)-6-oxo-5-((3-phenylpropyl)amino) pyrimidin-1(6H)-yl)acetate (177 mg, 0.42 mmol) and (4-(S-methyl-N-(2,2,2-trifluoroacetyl)sulfonimidoyl)phenyl)boronic acid (250 mg, 0.85 mmol) in THF (3 mL) was added CuTc (177 mg, 0.93 mmol) and Pd(PPh₃)₄ (98 mg, 0.085 mmol) under N₂ atmosphere, the reaction mixture was degassed under N₂ atmosphere for three times and stirred at 80° C. overnight. The mixture was diluted with water and extracted with EtOAc twice. The combined organic layers were washed with saturated aq.NaHCO₃ solution and brine, dried over anhydrous Na₂SO₄, filtered, and concentrated under reduced pressure to dryness. The residue was purified by flash chromatography (silica gel, 0-50% EtOAc in PE) to give the title compound (20 mg, yield 8.0%) as a brown oil. LC/MS (ESI) m/z: 593 (M+H)⁺.

### Step 3: 2-(2-(4-(S-methyl-N-(2,2,2-trifluoroacetyl)sulfonimidoyl)phenyl)-6-oxo-5-((3-phenylpropyl)amino)pyrimidin-1(6H)-yl)acetic acid (4)

A solution of tert-butyl 2-(2-(4-(S-methyl-N-(2,2,2-trifluoroacetyl)sulfonimidoyl) phenyl)-6-oxo-5-((3-phenylpropyl)amino)pyrimidin-1(6H)-yl)acetate (20 mg, 0.034 mmol) in TFA (1 mL) was stirred under N₂ atmosphere at room temperature for 2 hours. The reaction mixture was concentrated under reduced pressure to dryness to give the title compound (18 mg, yield 98.6%) as a yellow solid, which was used directly in the next step without further purification. LC/MS (ESI) m/z: 537 (M+H)⁺.

### Step 4: N-((4-(1-(2-(((1H-pyrrolo[3,2-c]pyridine-2-yl)methyl)amino)-2-oxoethyl)-6-oxo-5-((3-phenylpropyl)amino)-1,6-dihydropyrimidin-2-yl)phenyl)(methyl)(oxo)-l6-sulfanylidene)-2,2,2-trifluoroacetamide (5)

To a mixture of 2-(2-(4-(S-methyl-N-(2,2,2-trifluoroacetyl)sulfonimidoyl)phenyl)-6-oxo-5-((3-phenylpropyl)amino)pyrimidin-1(6H)-yl)acetic acid (18 mg, 0.034 mmol) and (1H-pyrrolo[3,2-c]pyridine-2-yl)methanamine hydrochloride (10 mg, 0.067 mmol) in DMF (1 mL) was added DIPEA (22 mg, 0.17 mmol) and HATU (19 mg, 0.051 mmol) under N₂ atmosphere and the reaction mixture was stirred at room temperature for 2 hours. The mixture was concentrated under reduced pressure to dryness. The residue was purified by prep-TLC (DCM:MeOH=10:1) to give the title compound (15 mg, yield 66.7%) as a brown solid. LC/MS (ESI) m/z: 666 (M+H)⁺.

### Step 5: N-((1H-pyrrolo[3,2-c]pyridine-2-yl)methyl)-2-(2-(4-(S-methylsulfonimidoyl) phenyl)-6-oxo-5-((3-phenylpropyl)amino)pyrimidin-1(6H)-yl)acetamide (Compound 200)

To a solution of N-((4-(1-(2-(((1H-pyrrolo[3,2-c]pyridine-2-yl)methyl)amino)-2-oxoethyl)-6-oxo-5-((3-phenylpropyl)amino)-1,6-dihydropyrimidin-2-yl)phenyl)(methyl) (oxo)-l6-sulfanylidene)-2,2,2-trifluoroacetamide (15 mg, 0.023 mmol) in MeOH (1 mL) was added K₂CO₃ (15 mg, 0.23 mmol) and the mixture was stirred at room temperature for 3 hours. The mixture was concentrated under reduced pressure to dryness. The residue was purified by prep-HPLC to give Compound 200 (1.0 mg, yield 7.6%) as a white solid. ¹H NMR (400 MHz, CD₃OD) δ 8.73 (s, 1H), 8.11 (d, J=5.8 Hz, 1H), 8.03 (d, J=8.4 Hz, 2H), 7.74 (d, J=8.4 Hz, 2H), 7.40 (d, J=5.8 Hz, 1H), 7.25 (dd, J=15.7, 7.0 Hz, 4H), 7.17 (s, 1H), 7.11 (s, 1H), 6.51 (s, 1H), 4.62 (s, 2H), 4.56 (s, 2H), 3.21 (d, J=7.0 Hz, 2H), 3.13 (s, 3H), 2.75 (t, J=7.6 Hz, 2H), 2.02-1.98 (m, 2H). LC/MS (ESI) m/z: 570 (M+H)⁺. RT (Method A): 1.22 min.

### Scheme 48. Synthesis of N-((1H-pyrrolo[3,2-c]pyridine-2-yl)methyl)-2-(2-(4-((1,1-dioxidothietan-3-yl)oxy)phenyl)-6-oxo-5-((3-phenylpropyl)amino)pyrimidin-1(6H)-yl)acetamide (Compound 124)

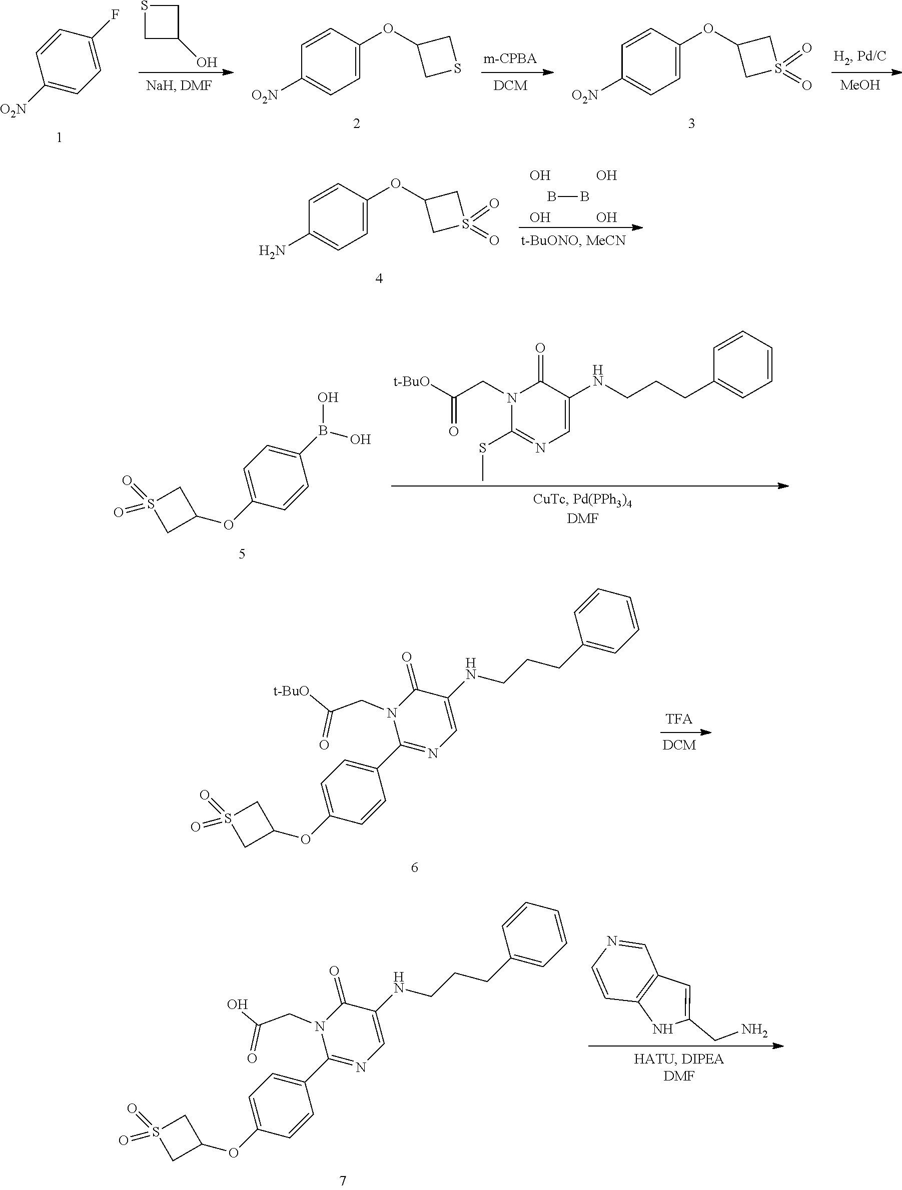

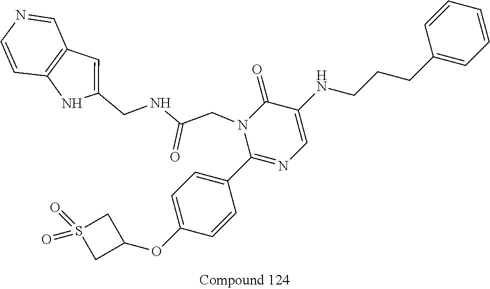 [SMILES: O=C(Cn1c(-c2ccc(OC3CS(=O)(=O)C3)cc2)ncc(NCCCc2ccccc2)c1=O)NCc1cc2cnccc2[nH]1]

### Step 1: 3-(4-Nitrophenoxy)thietane (2)

To a solution of thietan-3-ol (3.00 g, 33.33 mmol) in DMF (45 mL) was added 1-fluoro-4-nitrobenzene (6.24 g, 44.26 mmol) followed by portion-wise addition of NaH (1.73 g, 43.33 mmol, 60% dispersion in mineral oil) at 0° C. under N₂ atmosphere and the reaction mixture was stirred at room temperature overnight. The reaction mixture was quenched with saturated aq.NH₄Cl solution at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give the title compound (7.00 g, yield 99.6%) as a colorless oil. LC/MS (ESI) m/z: 212 (M+H)⁺.

### Step 2: 3-(4-Nitrophenoxy)thietane 1,1-dioxide (3)

To a solution of 3-(4-nitrophenoxy)thietane (6.0 g, 28.44 mmol) in DCM (30 mL) was added m-CPBA (14.7 g, 85.28 mmol) and the mixture was stirred at room temperature for 3 hours. The mixture was diluted with DCM, washed with saturated aq.Na₂S₂O₃ solution and brine, dried over anhydrous Na₂SO₄, filtered, and concentrated under reduced pressure to dryness. The residue was purified by flash chromatography (silica gel, 0-30% EtOAc in PE) to give the title compound (6.5 g, yield 94.1%) as a yellow solid. LC/MS (ESI) m/z: 244 (M+H)⁺.

### Step 3: 3-(4-Aminophenoxy)thietane 1,1-dioxide (4)

To a solution of methyl 3-(4-nitrophenoxy)thietane 1,1-dioxide (3.0 g, 12.35 mmol) in MeOH (30 mL) was added Pd/C (500 mg, 10% wt.), the mixture was degassed under N₂ atmosphere for three times and stirred under a H₂ balloon at room temperature for 1 hour. The mixture was filtered, and the filtrate was concentrated under reduced pressure to dryness. The residue was purified by flash chromatography (silica gel, 0-50% EtOAc in PE) to give the title compound (2.2 g, yield 82.5%) as a brown solid. LC/MS (ESI) m/z: 214 (M+H)⁺.

### Step 4: (4-((1,1-Dioxidothietan-3-yl)oxy)phenyl)boronic acid (5)

To a solution of 3-(4-aminophenoxy)thietane 1,1-dioxide (600 mg, 2.82 mmol) in MeCN (15 mL) was added t-BuONO (1450 mg, 14.1 mmol) and hypodiboric acid (758 mg, 8.45 mmol) under N₂ atmosphere at 0° C. and the reaction mixture was stirred at room temperature for 5 hours. The mixture was diluted with DCM, washed with water and brine, dried over anhydrous Na₂SO₄, filtered, and concentrated under reduced pressure to dryness. The residue was purified by flash chromatography (silica gel, 0-10% MeOH in DCM) to give the title compound (128 mg, yield 18.8%) as a brown solid. ¹H NMR (400 MHz, DMSO-d₆) δ 7.92 (s, 2H), 7.76 (d, J=8.6 Hz, 2H), 6.88 (d, J=8.6 Hz, 2H), 5.17 (td, J=7.0, 4.0 Hz, 1H), 4.84-4.77 (m, 2H), 4.22 (dd, J=15.7, 2.8 Hz, 2H).

### Step 5: Tert-butyl 2-(2-(4-((1,1-Dioxidothietan-3-yl)oxy)phenyl)-6-oxo-5-((3-phenylpropyl)amino)pyrimidin-1(6H)-yl)acetate (6)

To a mixture of (4-((1,1-dioxidothietan-3-yl)oxy)phenyl)boronic acid (103 mg, 0.43 mmol) and tert-butyl 2-(2-(methylthio)-6-oxo-5-((3-phenylpropyl)amino)pyrimidin-1(6H)-yl)acetate (113 mg, 0.29 mmol) in DMF (4 mL) was added CuTc (174 mg, 0.91 mmol) and Pd(PPh₃)₄ (48 mg, 0.04 mmol) under N₂ atmosphere. The mixture was degassed under N₂ atmosphere for three times and stirred in a sealed tube at 80° C. overnight. The reaction mixture was diluted with EtOAc and filtered. The filtrate was washed with saturated aq.NaHCO₃ solution and brine, dried over anhydrous Na₂SO₄, filtered, and concentrated under reduced pressure to dryness. The residue was purified by prep-TLC (PE:EtOAc=3:1) to give the title compound (50 mg, yield 21.8%) as a brown solid. LC/MS (ESI) m/z: 540 (M+H)⁺.

### Step 6: 2-(2-(4-((1,1-Dioxidothietan-3-yl)oxy)phenyl)-6-oxo-5-((3-phenylpropyl) amino)pyrimidin-1(6H)-yl)acetic acid (7)

To a solution of tert-butyl 2-(2-(4-((1,1-dioxidothietan-3-yl)oxy)phenyl)-6-oxo-5-((3-phenylpropyl)amino)pyrimidin-1(6H)-yl)acetate (50 mg, 0.09 mmol) in DCM (2 mL) was added TFA (1 mL) and the mixture was stirred at room temperature for 1 hour. The mixture was concentrated under reduced pressure to dryness to give the title compound (30 mg, yield 67.0%) as a brown oil, which was used directly in the next step without further purification. LC/MS (ESI) m/z: 484 (M+H)⁺.

### Step 7: N-((1H-pyrrolo[3,2-c]pyridine-2-yl)methyl)-2-(2-(4-((1,1-dioxidothietan-3-yl)oxy)phenyl)-6-oxo-5-((3-phenylpropyl)amino)pyrimidin-1(6H)-yl)acetamide (Compound 124)

To a mixture of 2-(2-(4-((1,1-dioxidothietan-3-yl)oxy)phenyl)-6-oxo-5-((3-phenylpropyl)amino)pyrimidin-1(6H)-yl)acetic acid (30 mg, 0.06 mmol) and (1H-pyrrolo[3,2-c]pyridine-2-yl)methanamine (14 mg, 0.10 mmol) in DMF (1 mL) was added DIPEA (48 mg, 0.37 mmol) and HATU (43 mg, 0.11 mmol) under N₂ atmosphere and the reaction mixture was stirred at room temperature for 1 hour. The mixture was diluted with EtOAc, washed with saturated aq.NH₄Cl solution and brine, dried over anhydrous Na₂SO₄, filtered, and concentrated under reduced pressure to dryness. The residue was purified by prep-TLC (DCM:MeOH=8:1) and further purified by prep-HPLC to give Compound 124 (3.6 mg, yield 9.5%) as a colorless solid. ¹H NMR (400 MHz, CD₃OD) δ 8.74 (s, 1H), 8.13 (d, J=5.8 Hz, 1H), 7.48-7.40 (m, 3H), 7.31-7.22 (m, 4H), 7.17 (t, J=7.3 Hz, 1H), 7.08 (s, 1H), 6.89 (d, J=8.5 Hz, 2H), 6.51 (s, 1H), 5.13-5.06 (m, 1H), 4.71-4.62 (m, 4H), 4.55 (s, 2H), 4.22-4.15 (m, 2H), 3.18 (t, J=6.9 Hz, 2H), 2.75 (t, J=7.7 Hz, 2H), 2.03-1.96 (m, 2H). LC/MS (ESI) m/z: 613 (M+H)⁺. RT (Method A): 1.43 min.

### Scheme 49. Synthesis of N-((1H-pyrrolo[3,2-c]pyridine-2-yl)methyl)-2-(2-(4-(((3R,3aR,6R,6aR)-6-hydroxyhexahydrofuro[3,2-b]furan-3-yl)oxy)phenyl)-6-oxo-5-((3-phenylpropyl) amino)pyrimidin-1(6H)-yl)acetamide (Compound 125)

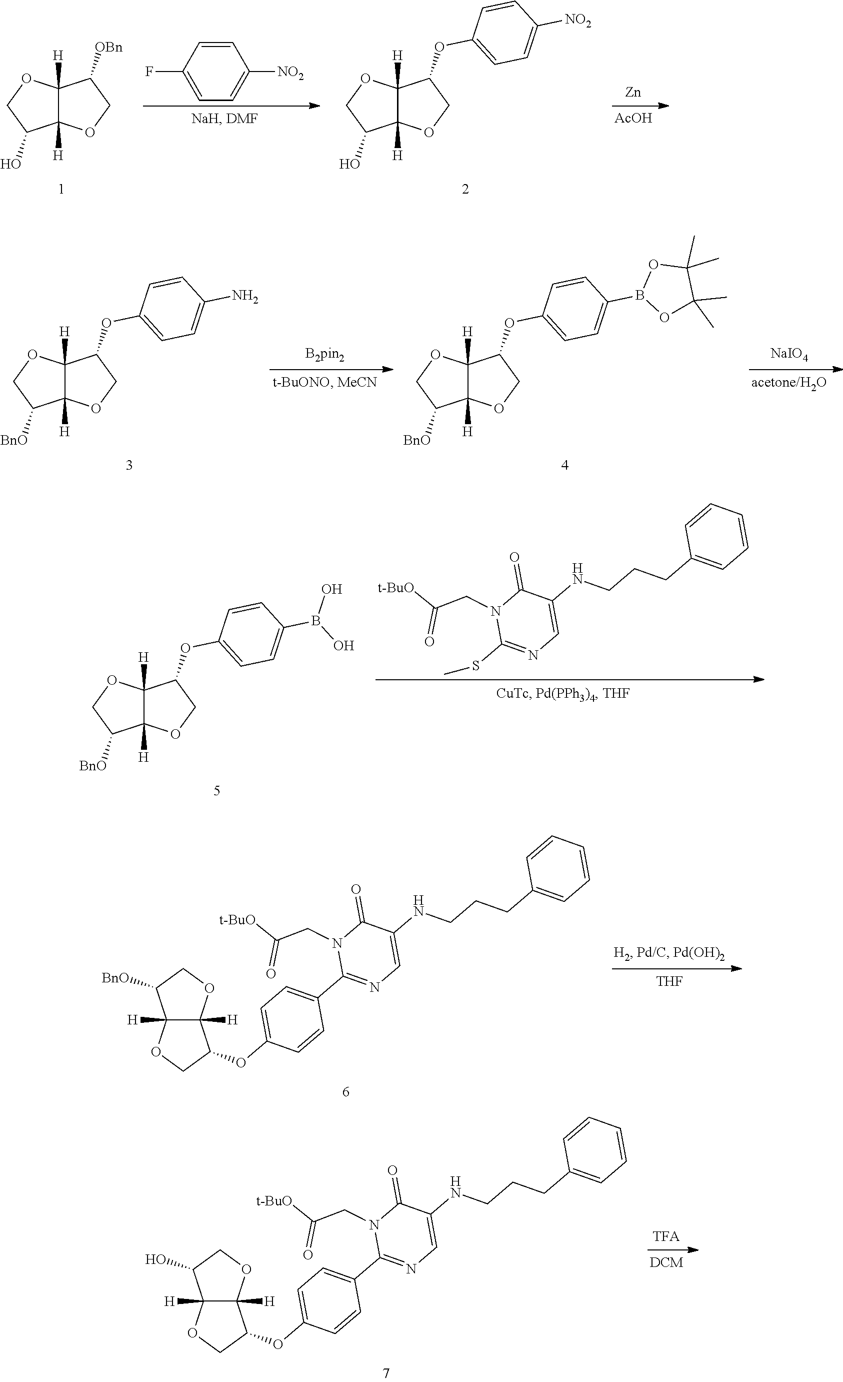

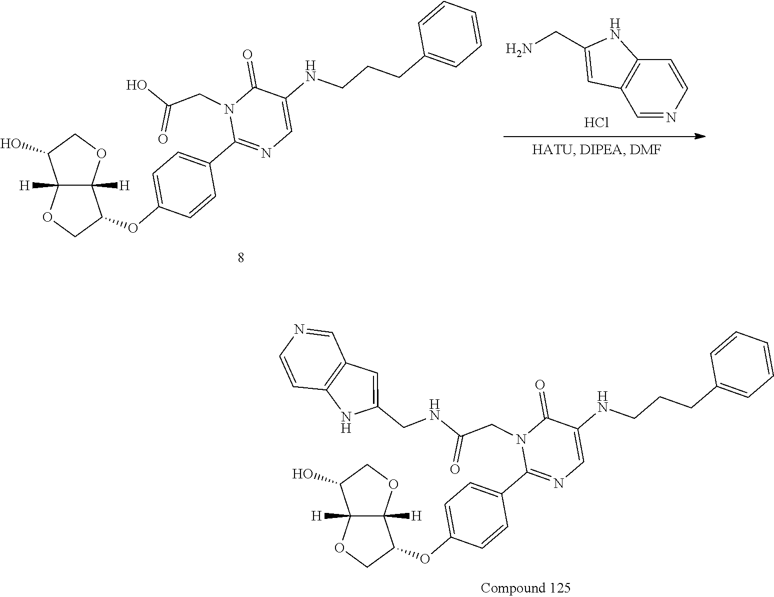

### Step 1: (3R,3aR,6R,6aR)-3-(Benzyloxy)-6-(4-nitrophenoxy)hexahydrofuro[3,2-b]furan (2)

To a solution of (3R,3aR,6R,6aR)-6-(benzyloxy)hexahydrofuro[3,2-b]furan-3-ol (1.0 g, 4.23 mmol) in dry DMF (10 mL) was added NaH (122 mg, 5.08 mmol, 60% dispersion in mineral oil) in portions at 0° C. and the mixture was stirred at 0° C. for 0.5 hour. And then, to the reaction mixture was added 1-fluoro-4-nitrobenzene (780 mg, 5.52 mmol) portion-wise at 0° C. and the reaction mixture was stirred at room temperature for 3 hours. The mixture was quenched with saturated aq.NH₄Cl solution and extracted with EtOAc twice. The combined organic layers were washed with brine, dried over anhydrous Na₂SO₄, filtered, and concentrated to dryness. The residue was purified by flash chromatography (silica gel, 0-50% EtOAc in PE) to give the title compound (1.2 g, yield 79.3%) as a yellow oil. LC/MS (ESI) m/z: 358 (M+H)⁺.

### Step 2: 4-(((3R,3aR,6R,6aR)-6-(Benzyloxy)hexahydrofuro[3,2-b]furan-3-yl)oxy) aniline (3)

To a solution of (3R,3aR,6R,6aR)-3-(benzyloxy)-6-(4-nitrophenoxy)hexahydrofuro[3,2-b]furan (1.2 g, 3.35 mmol) in AcOH (12 mL) was added Zn (874 mg, 13.45 mmol) under N₂ atmosphere and the reaction mixture was stirred at 50° C. overnight. The mixture was filtered, and the filtrate was concentrated to dryness. The residue was purified by flash chromatography (silica gel, 0-94% EtOAc in PE) to give the title compound (450 mg, yield 40.9%) as a yellow oil. LC/MS (ESI) m/z: 328 (M+H)⁺.

### Step 3: 2-(4-(((3R,3aR,6R,6aR)-6-(Benzyloxy)hexahydrofuro[3,2-b]furan-3-yl)oxy) phenyl)-4,4,5,5-tetramethyl-1,3,2-dioxaborolane (4)

To a solution of 4-(((3R,3aR,6R,6aR)-6-(benzyloxy)hexahydrofuro[3,2-b]furan-3-yl)oxy)aniline (450 mg, 1.37 mmol) in MeCN (5 mL) was added t-BuONO (425 mg, 4.12 mmol) followed by 4,4,4′,4′,5,5,5′,5′-octamethyl-2,2′-bi(1,3,2-dioxaborolane) (524 mg, 2.06 mmol) under N₂ atmosphere and the reaction mixture was degassed under N₂ atmosphere for three times and stirred at room temperature for 3 hours. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give the title compound (330 mg, yield 54.8%) as a yellow oil. LC/MS (ESI) m/z: 439 (M+H)⁺.

### Step 4: (4-(((3R,3aR,6R,6aR)-6-(Benzyloxy)hexahydrofuro[3,2-b]furan-3-yl)oxy) phenyl)boronic acid (5)

To a solution of 2-(4-(((3R,3aR,6R,6aR)-6-(benzyloxy)hexahydrofuro[3,2-b]furan-3-yl)oxy)phenyl)-4,4,5,5-tetramethyl-1,3,2-dioxaborolane (300 mg, 0.68 mol) in acetone/water (4 mL, v/v=3/1) was added NaIO₄ (731 mg, 3.41 mmol) at 0° C. and the reaction mixture was stirred at room temperature overnight. The mixture was diluted with EtOAc, washed with water and brine, dried over anhydrous Na₂SO₄, filtered, and concentrated under reduced pressure to dryness. The residue was purified by prep-HPLC to give the title compound (220 mg, yield 90.2%) as a yellow solid. LC/MS (ESI) m/z: 357 (M+H)⁺.

### Step 5: Tert-butyl 2-(2-(4-(((3R,3aR,6R,6aR)-6-(benzyloxy)hexahydrofuro[3,2-b]furan-3-yl)oxy)phenyl)-6-oxo-5-((3-phenylpropyl)amino)pyrimidin-1(6H)-yl)acetate (6)

To a mixture of (4-(((3R,3aR,6R,6aR)-6-(benzyloxy)hexahydrofuro[3,2-b]furan-3-yl)oxy)phenyl)boronic acid (220 mg, 0.62 mmol) and tert-butyl 2-(2-(methylthio)-6-oxo-5-((3-phenylpropyl)amino)pyrimidin-1(6H)-yl)acetate (240 mg, 0.62 mmol) in THF (3 mL) was added CuTc (260 mg, 1.36 mmol) and Pd(PPh₃)₄ (142 mg, 0.12 mmol) under N₂ atmosphere and the reaction mixture was degassed under N₂ atmosphere for three times and stirred in a sealed tube at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by prep-HPLC to give the title compound (55 mg, yield 13.6%) as a white solid. LC/MS (ESI) m/z: 654 (M+H)⁺.

### Step 6: Tert-butyl 2-(2-(4-(((3R,3aR,6R,6aR)-6-hydroxyhexahydrofuro[3,2-b]furan-3-yl)oxy)phenyl)-6-oxo-5-((3-phenylpropyl)amino)pyrimidin-1(6H)-yl)acetate (7)

To a solution of tert-butyl 2-(2-(4-(((3R,3aR,6R,6aR)-6-(benzyloxy)hexahydrofuro[3,2-b]furan-3-yl)oxy)phenyl)-6-oxo-5-((3-phenylpropyl)amino)pyrimidin-1(6H)-yl)acetate (55 mg, 0.084 mmol) in THF (3 mL) was added Pd/C (20 mg, 10% wt.) and Pd(OH)₂ (20 mg, 10% wt.), the reaction mixture was degassed under N₂ atmosphere for three times and stirred under a H₂ balloon at 50° C. for 2 days. The mixture was filtered, and the filtrate was concentrated to dryness to give the title compound (45 mg, yield 94.9%) as a white solid, which was used directly in the next step without further purification. LC/MS (ESI) m/z: 564 (M+H)⁺.

### Step 7: 2-(2-(4-(((3R,3aR,6R,6aR)-6-hydroxyhexahydrofuro[3,2-b]furan-3-yl)oxy) phenyl)-6-oxo-5-((3-phenylpropyl)amino)pyrimidin-1(6H)-yl)acetic acid (8)

To a solution of tert-butyl 2-(2-(4-(((3R,3aR,6R,6aR)-6-hydroxyhexahydrofuro[3,2-b]furan-3-yl)oxy)phenyl)-6-oxo-5-((3-phenylpropyl)amino)pyrimidin-1(6H)-yl)acetate (35 mg, 0.062 mol) in DCM (1 mL) was added TFA (1 mL) at 0° C. and the mixture was stirred at room temperature for 5 hours. The mixture was concentrated under reduced pressure to give the title compound (30 mg, yield 95.1%) as a yellow oil, which was used in next reaction without purification. LC/MS (ESI) m/z: 508 (M+H)⁺.

### Step 8: N-((1H-pyrrolo[3,2-c]pyridine-2-yl)methyl)-2-(2-(4-(((3R,3aR,6R,6aR)-6-hydroxyhexahydrofuro[3,2-b]furan-3-yl)oxy)phenyl)-6-oxo-5-((3-phenylpropyl) amino)pyrimidin-1(6H)-yl)acetamide (Compound 125)

To a mixture of 2-(2-(4-(((3R,3aR,6R,6aR)-6-hydroxyhexahydrofuro[3,2-b]furan-3-yl)oxy)phenyl)-6-oxo-5-((3-phenylpropyl)amino)pyrimidin-1(6H)-yl)acetic acid (30 mg, 0.059 mmol) and (1H-pyrrolo[3,2-c]pyridine-2-yl)methanamine hydrochloride (13 mg, 0.071 mmol) in DMF (1 mL) was added DIPEA (39 mg, 0.30 mmol) and HATU (27 mg, 0.071 mmol) under N₂ atmosphere and the mixture was stirred at room temperature for 1 hour. The mixture was concentrated under reduced pressure to dryness. The residue was purified by prep-HPLC to give Compound 125 (6.2 mg, yield 16.4%) as a white solid. ¹H NMR (400 MHz, CD₃OD) δ 8.73 (s, 1H), 8.12 (d, J=5.8 Hz, 1H), 7.41 (d, J=5.8 Hz, 1H), 7.38 (d, J=8.6 Hz, 2H), 7.29-7.24 (m, 2H), 7.22 (d, J=7.1 Hz, 2H), 7.16 (t, J=7.1 Hz, 1H), 7.06 (s, 1H), 6.93 (d, J=8.5 Hz, 2H), 6.50 (s, 1H), 4.78 (d, J=5.5 Hz, 1H), 4.72 (t, J=4.9 Hz, 1H), 4.63 (d, J=2.2 Hz, 2H), 4.54 (s, 2H), 4.47 (t, J=4.9 Hz, 1H), 4.27-4.22 (m, 1H), 4.08 (dd, J=9.4, 6.0 Hz, 1H), 3.91 (dd, J=9.3, 5.8 Hz, 1H), 3.84-3.80 (m, 1H), 3.54 (t, J=8.5 Hz, 1H), 3.16 (t, J=6.5 Hz, 2H), 2.74 (t, J=7.6 Hz, 2H), 2.02-1.95 (m, 2H), 1.31 (d, J=19.0 Hz, 1H). LC/MS (ESI) m/z: 637 (M+H)⁺. RT (Method A): 1.28 min.

Compound 288 was prepared based on Steps 5-8 in Scheme 49:

| # | Reactant A | Reactant B | Reactant C | Characterization Data |
|---|---|---|---|---|
| 288ª | 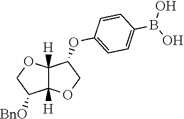 [SMILES: OB(O)c1ccc(O[C@@H]2CO[C@H]3[C@@H]2OC[C@H]3OCc2ccccc2)cc1] | 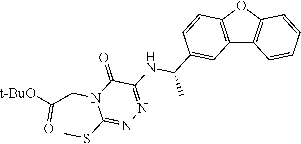 [SMILES: CSc1nnc(N[C@@H](C)c2ccc3oc4ccccc4c3c2)c(=O)n1CC(=O)OC(C)(C)C] | 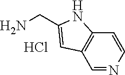 [SMILES: NCc1cc2cnccc2[nH]1 ; Cl] | ¹H NMR (400 MHz, DMSO- d₆) δ 11.33 (s, 1H), 8.75 (s, 1H), 8.71 (t, J = 5.6 Hz, 1H), 8.22 (s, 1H), 8.13-8.09 (m, 2H), 7.70-7.61 (m, 3H), 7.51 (t, J = 7.3 Hz, 1H), 7.40 (t, J = 7.5 Hz, 1H), 7.34 (d, J = 5.6 Hz, 1H), 7.29 (d, J = 8.7 Hz, 2H), 6.97 (s, 1H), 6.89 (d, J = 8.8 Hz, 2H), |
| | | | | 6.36 (s, 1H), 5.87 (d, J = 7.0 Hz, 1H), |
| | | | | 4.90 (d, J = 6.5 Hz, 1H), 4.77 (dd, J = |
| | | | | 11.8, 6.3 Hz, 1H), 4.66 (dd, J = |
| | | | | 9.1, 4.5 Hz, 2H), 4.54 (d, |
| | | | | J = 16.1 Hz, 1H), 4.43 |
| | | | | (dd, J = 10.7, 5.1 Hz, 3H), 4.12- |
| | | | | 4.34 (t, J = 4.9 Hz, 1H), 4.06 (m, |
| | | | | 1H), 4.01 (dd, J = 9.0, 6.4 Hz, |
| | | | | 1H), 3.76-3.69 (m, 2H), 3.36 (d, |
| | | | | J = 8.5 Hz, 1H), 1.60 (d, J = 6.7 |
| | | | | Hz, 3H). LC/MS (ESI) m/z: 713 (M + |
| | | | | H)⁺. RT (Method A): 1.52 min. |

^{a}step 5 was performed in DMF, Step 6 was performed in THF, and Step 7 was performed with LiOH in MeOH/H₂O.

### Scheme 50. Synthesis of N-((1H-pyrrolo[3,2-c]pyridine-2-yl)methyl)-2-(5-((dibenzo[b,d]furan-2-ylmethyl)amino)-2-(4-fluorophenyl)-6-oxopyrimidin-1(6H)-yl)acetamide (Compound 123)

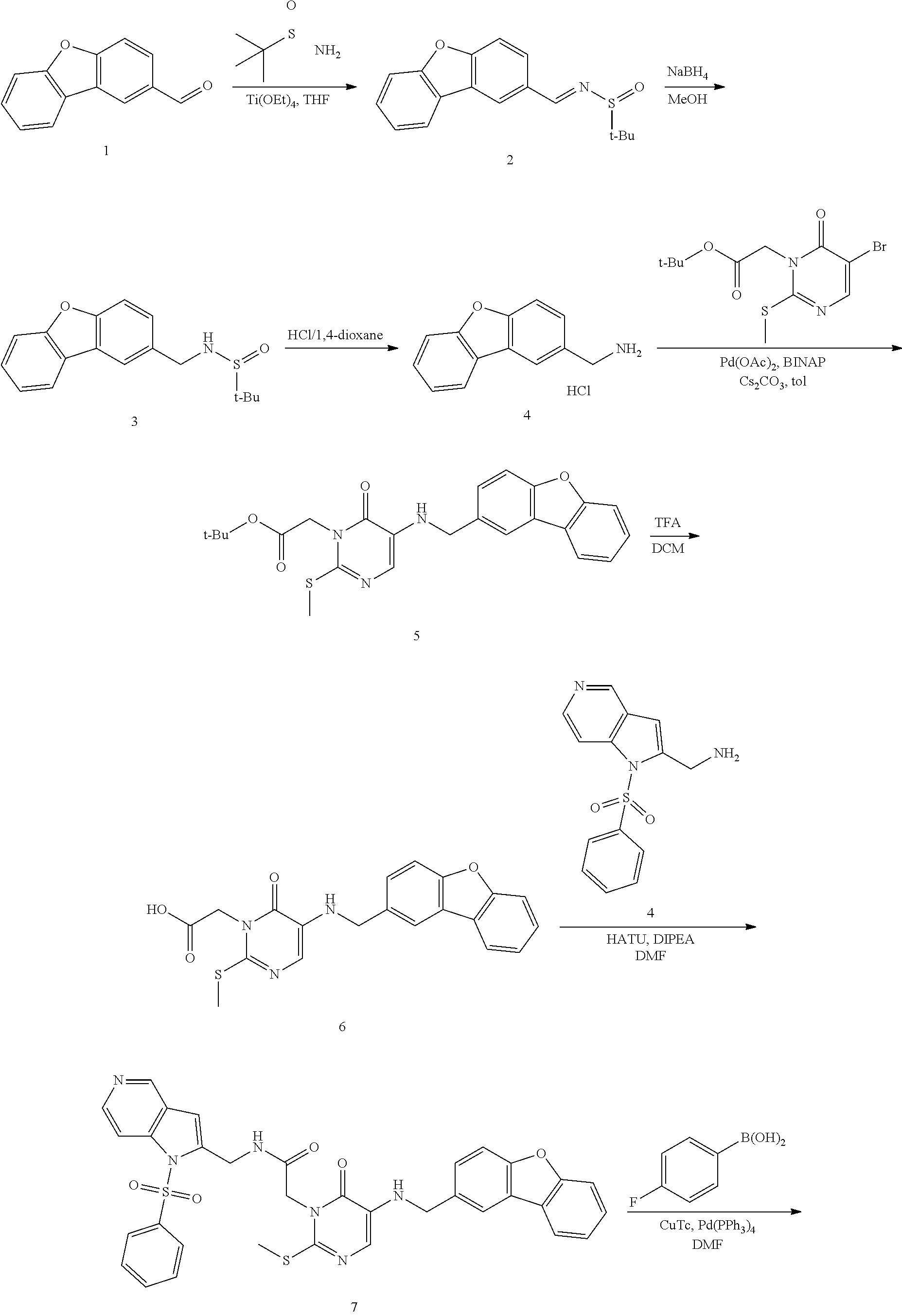

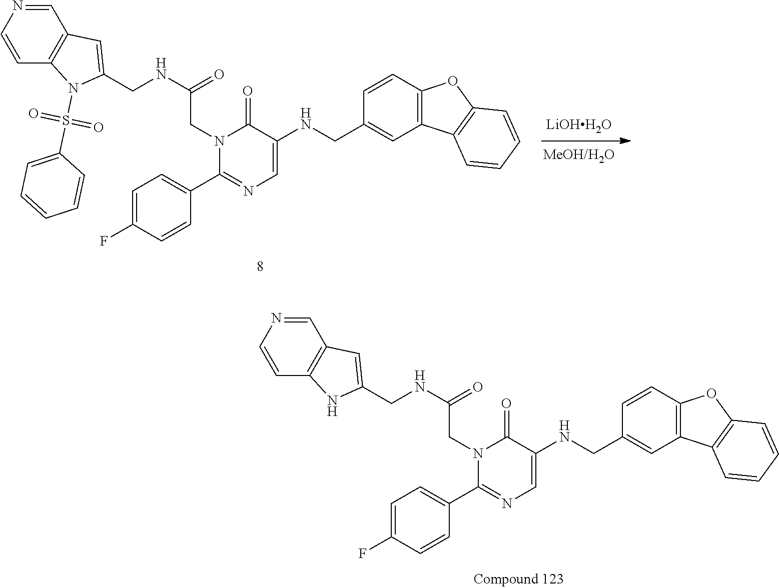

### Step 1: (E)-N-(dibenzo[b,d]furan-2-ylmethylene)-2-methylpropane-2-sulfinamide (2)

To a mixture of dibenzo[b,d]furan-2-carbaldehyde (10.4 g, 53 mmol) and 2-methylpropane-2-sulfinamide (12.8 g, 106 mmol) in THF (100 mL) was added Ti(OEt)₄ under N₂ atmosphere and the reaction mixture was stirred at 70° C. for 30 minutes. The mixture was quenched with water and filtered. The filtrate was extracted with EtOAc twice. The combined organic layers were dried over anhydrous Na₂SO₄, filtered, and concentrated under reduced pressure to give the title compound (15 g, 94.7% yield) as a yellow solid, which was used in the next step without further purification. LC/MS (ESI) m/z: 300 (M+H)⁺.

### Step 2: N-(dibenzo[b,d]furan-2-ylmethyl)-2-methylpropane-2-sulfinamide (3)

To a solution of (E)-N-(dibenzo[b,d]furan-2-ylmethylene)-2-methylpropane-2-sulfinamide (15 g, 50.2 mmol) in MeOH (150 mL) was added NaBH₄ (7.5 g, 198 mmol) under N₂ atmosphere and the reaction mixture was stirred at room temperature for 10 minutes. The mixture was quenched with 0.5 N aq. HCl solution and extracted with EtOAc twice. The combined organic layer was dried over anhydrous Na₂SO₄, filtered, and concentrated under reduced pressure to dryness. The residue was purified by flash chromatography (silica gel, 0-50% EtOAc in PE) to give the title compound (12 g, yield 79.5%) as a white solid. LC/MS (ESI) m/z: 302 (M+H)⁺.

### Step 3: Dibenzo[b,d]furan-2-ylmethanamine hydrochloride (4)

To a solution of N-(dibenzo[b,d]furan-2-ylmethyl)-2-methylpropane-2-sulfinamide (15 g, 49.8 mmol) in HCl/1,4-dioxane (4 M, 80 mL) was stirred under N₂ atmosphere at room temperature for 2 hours. The reaction mixture was concentrated under reduced pressure to dryness to give the title compound (11.4 g, yield 98.4%) as a yellow oil, which was used directly in the next step without further purification. LC/MS (ESI) m/z: 198 (M+H)⁺.

### Step 4: Tert-butyl 2-(5-((dibenzo[b,d]furan-2-ylmethyl)amino)-2-(methylthio)-6-oxopyrimidin-1(6H)-yl)acetate (5)

To a solution of dibenzo[b,d]furan-2-ylmethanamine hydrochloride (9 g, 45.7 mmol) in toluene (90 mL) was added tert-butyl 2-(5-bromo-2-(methylthio)-6-oxopyrimidin-1(6H)-yl)acetate (15 g, 45.7 mmol), Pd(OAc)₂ (1 g, 4.6 mmol), BINAP (5.7 g, 9.2 mmol) and Cs₂CO₃ (37 g, 114.2 mmol) under N₂ atmosphere, the mixture was degassed under N₂ atmosphere for three times and stirred at 120° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (9 g, yield 43.8%) as a yellow solid.

### Step 5: 2-(5-((dibenzo[b,d]furan-2-ylmethyl)amino)-2-(methylthio)-6-oxopyrimidin-1(6H)-yl)acetic acid (6)

To a solution of tert-butyl 2-(5-((dibenzo[b,d]furan-2-ylmethyl)amino)-2-(methylthio)-6-oxopyrimidin-1(6H)-yl)acetate (4.5 g, 10 mmol) in DCM (10 mL) was added TFA (10 mL) under N₂ atmosphere and the reaction mixture was stirred at room temperature for 1 hour. The mixture was diluted with DCM and washed with water twice. The organic layer was dried over anhydrous Na₂SO₄, filtered, and concentrated under reduced pressure to dryness to give the title compound (3.9 g, yield 98%) as a yellow solid, which was used directly in the next step without further purification. LC/MS (ESI) m/z: 396 (M+H)⁺.

### Step 6: 2-(5-((Dibenzo[b,d]furan-2-ylmethyl)amino)-2-(methylthio)-6-oxopyrimidin-1(6H)-yl)-N-((1-(phenylsulfonyl)-1H-pyrrolo[3,2-c]pyridine-2-yl)methyl)acetamide (7)

To a mixture of 2-(5-((dibenzo[b,d]furan-2-ylmethyl)amino)-2-(methylthio)-6-oxopyrimidin-1(6H)-yl)acetic acid (500 mg, 1.3 mmol) and (1-(phenylsulfonyl)-1H-pyrrolo[3,2-c]pyridine-2-yl)methanamine (373 mg, 1.3 mmol) in DMF (6 mL) was added HATU (570 mg, 1.5 mmol) and DIPEA (671 mg, 5.2 mmol) under N₂ atmosphere and the reaction mixture was stirred at room temperature for 10 minutes. The mixture was concentrated under reduced pressure to dryness and the residue was purified by flash chromatography (silica gel, 0-10% MeOH in DCM) to give the title compound (150 mg, yield 17.4%) as a yellow solid. LC/MS (ESI) m/z: 665 (M+H)⁺.

### Step 7: 2-(5-((dibenzo[b,d]furan-2-ylmethyl)amino)-2-(4-fluorophenyl)-6-oxopyrimidin-1(6H)-yl)-N-((1-(phenylsulfonyl)-1H-pyrrolo[3,2-c]pyridine-2-yl)methyl) acetamide (8)

To a solution of 2-(5-((dibenzo[b,d]furan-2-ylmethyl)amino)-2-(methylthio)-6-oxopyrimidin-1(6H)-yl)-N-((1-(phenylsulfonyl)-1H-pyrrolo[3,2-c]pyridine-2-yl)methyl) acetamide (50 mg, 0.075 mmol) in DMF (1.5 mL) was added (4-fluorophenyl)boronic acid (21 mg, 0.15 mmol), Pd(PPh₃)₄ (17 mg, 0.015 mmol) and CuTc (31 mg, 0.16 mmol) under N₂ atmosphere and the reaction mixture was stirred at 80° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-60% EtOAc in PE) to give the title compound (15 mg, yield 28.1%) as a yellow solid. LC/MS (ESI) m/z: 713 (M+H)⁺.

### Step 8: N-((1H-pyrrolo[3,2-c]pyridine-2-yl)methyl)-2-(5-((dibenzo[b,d]furan-2-ylmethyl)amino)-2-(4-fluorophenyl)-6-oxopyrimidin-1(6H)-yl)acetamide (Compound 123)

To a solution of 2-(5-((dibenzo[b,d]furan-2-ylmethyl)amino)-2-(4-fluorophenyl)-6-oxopyrimidin-1(6H)-yl)-N-((1-(phenylsulfonyl)-1H-pyrrolo[3,2-c]pyridine-2-yl)methyl) acetamide (15 mg, 0.021 mmol) in MeOH/water (1.5 mL, v/v=1/1) was added LiOH·H₂O (31 mg, 0.16 mmol) and the reaction mixture was stirred at 60° C. for 1 hour. The mixture concentrated to dryness and the residue was purified by prep-HPLC to give Compound 123 (2 mg, yield 16.6%) as a white solid. ¹H NMR (400 MHz, CD₃OD) δ 8.71 (s, 1H), 8.10 (dd, J=8.8, 6.1 Hz, 2H), 8.00 (d, J=7.8 Hz, 1H), 7.63-7.51 (m, 3H), 7.50-7.45 (m, 3H), 7.40-7.33 (m, 2H), 7.11-7.05 (m, 3H), 6.48 (s, 1H), 5.35 (dd, J=9.6, 4.9 Hz, 2H), 4.59 (s, 2H), 4.55 (s, 2H). LC/MS (ESI) m/z: 573 (M+H)⁺. RT (Method A: 1.76 min.

### Scheme 51. Synthesis of (S)—N-((1H-pyrrolo[3,2-c]pyridine-2-yl)methyl)-2-(5-((1-(dibenzo[b,d]furan-2-yl)ethyl)amino)-6-oxo-2-phenylpyrimidin-1(6H)-yl)acetamide (Compound 127)

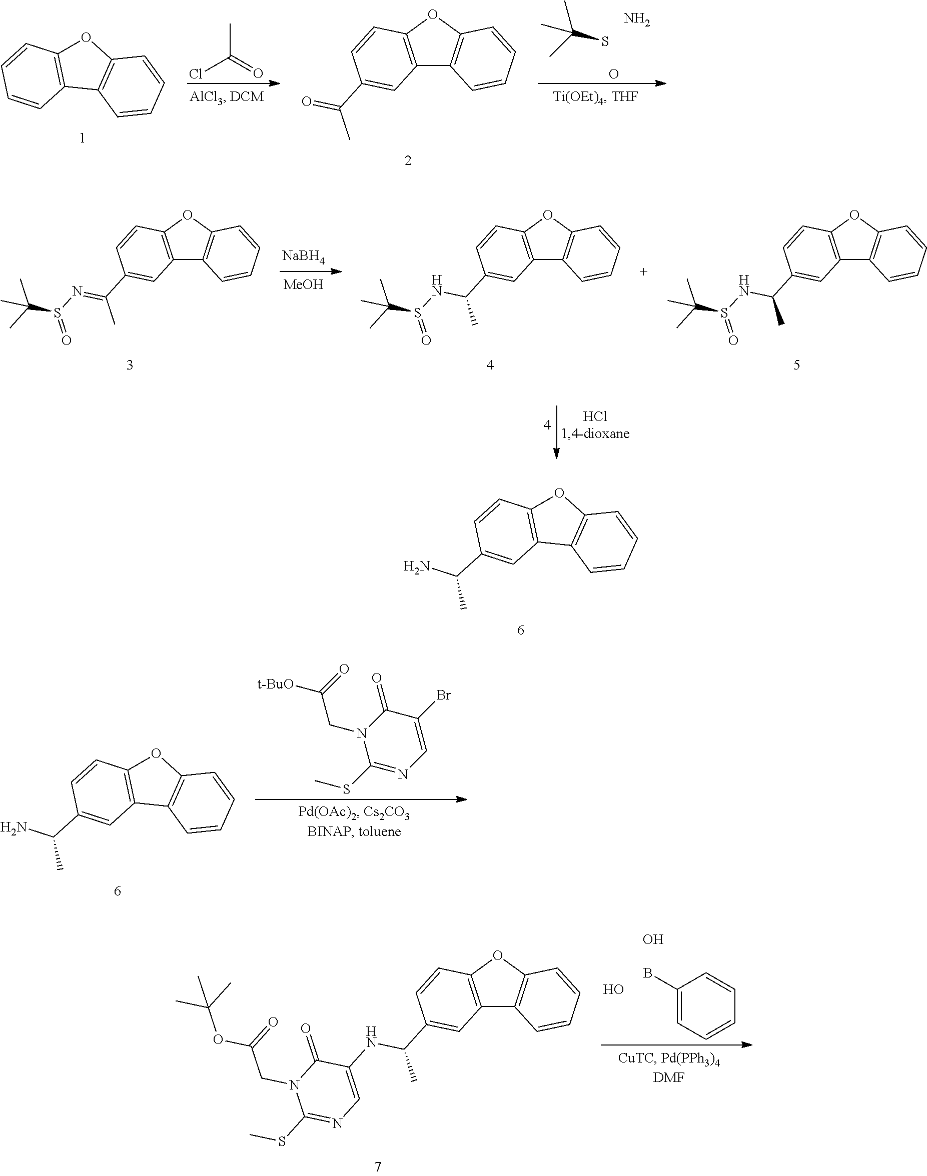

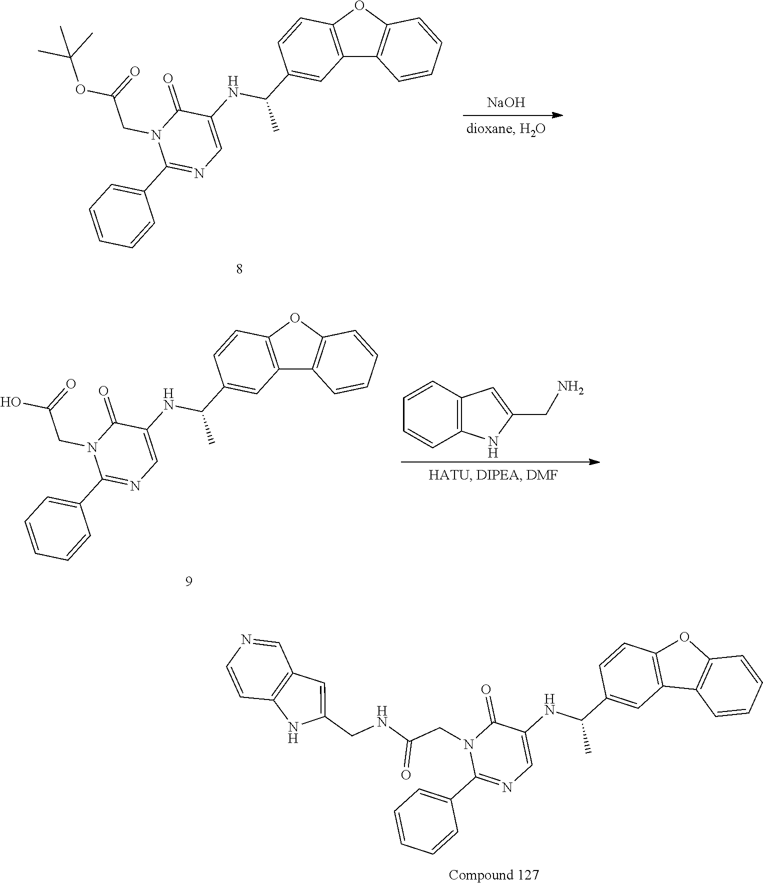

### Step 1: 1-(Dibenzo[b,d]furan-2-yl)ethan-1-one (2)

To a mixture of dibenzo[b,d]furan (1.5 g, 8.93 mmol) and acetyl chloride (1.08 g, 13.8 mmol) in DCM (15 mL) was added AlCl₃ (1.78 g, 13.3 mmol) at 0° C. under N₂ atmosphere and the reaction mixture was stirred under N₂ atmosphere at room temperature overnight. The mixture was quenched with ice-water and extracted with DCM twice. The combined organic layers were washed with brine, dried over anhydrous Na₂SO₄, filtered, and concentrated under reduced pressure to dryness. The residue was purified by flash chromatography (silica gel, 0-15% EtOAc in PE) to give the title compound (1.7 g, yield 90.9%) as a white solid.

### Step 2: (S,E)-N-(1-(dibenzo[b,d]furan-2-yl)ethylidene)-2-methylpropane-2-sulfinamide (3)

To a mixture of 1-(dibenzo[b,d]furan-2-yl)ethan-1-one (1.7 g, 8.09 mol) and S-(tert-butyl)thiohydroxylamine (2.48 g, 20.5 mmol) in THF (18 mL) was added Ti(OEt)₄ (7.47 g, 32.7 mmol) at 0° C. under N₂ atmosphere and the reaction mixture was stirred under N₂ atmosphere at 90° C. overnight. The mixture was quenched with ice-water and filtered. The filtrate was extracted with EtOAc twice. The combined organic layers were washed with brine, dried over anhydrous Na₂SO₄, filtered, and concentrated under reduced pressure to dryness to give the title compound (2.5 g, yield 98.6%) as a yellow solid, which was used directly in the next step without further purification. LC/MS (ESI) m/z: 314 (M+H)⁺.

### Step 3: (S)—N—((S)-1-(dibenzo[b,d]furan-2-yl)ethyl)-2-methylpropane-2-sulfinamide (4) and (S)—N—(®-1-(dibenzo[b,d]furan-2-yl)ethyl)-2-methylpropane-2-sulfinamide (5)

To a solution of (S,E)-N-(1-(dibenzo[b,d]furan-2-yl)ethylidene)-2-methylpropane-2-sulfinamide (2.5 g, 7.98 mmol) in MeOH (25 mL) was added NaBH₄ (755 mg, 19.9 mmol) in portions under N₂ atmosphere at 0° C. and the reaction mixture was stirred at room temperature for 4 hours. The mixture was quenched with 0.5 N aq. HCl and extracted with EtOAc twice. The combined organic layers were washed with brine, dried over anhydrous Na₂SO₄, filtered, and concentrated under reduced pressure to dryness. The residue was purified by flash chromatography (silica gel, 0-60% EtOAc in PE) to give (S)—N—((S)-1-(dibenzo[b,d]furan-2-yl)ethyl)-2-methylpropane-2-sulfinamide (4) (1.3 g, yield 51.7%) as a yellow solid. LC/MS (ESI) m/z: 316 (M+H)*, and (S)—N—(®-1-(dibenzo[b,d]furan-2-yl)ethyl)-2-methylpropane-2-sulfinamide (6) (1.3 g, yield 45%) as a yellow solid. LC/MS (ESI) m/z: 316 (M+H)⁺.

### Step 4: (S)-1-(dibenzo[b,d]furan-2-yl)ethan-1-amine (6)

A solution of (S)—N—((S)-1-(dibenzo[b,d]furan-2-yl)ethyl)-2-methylpropane-2-sulfinamide (1.3 g, 4.13 mmol) in HCl/1,4-dioxane (15 mL, 4M) was stirred under N₂ atmosphere at room temperature for 2 hours. The reaction mixture was concentrated under reduced pressure to dryness to give the title compound (850 mg, yield 97.5%) as a yellow solid, which was used directly in the next step without further purification. LC/MS (ESI) m/z: 212 (M+H)⁺.

### Step 5: Tert-butyl (S)-2-(5-((1-(dibenzo[b,d]furan-2-yl)ethyl)amino)-2-(methylthio)-6-oxopyrimidin-1(6H)-yl)acetate (7)

To a mixture of (S)-1-(dibenzo[b,d]furan-2-yl)ethan-1-amine (850 mg, 4.03 mmol) and tert-butyl 2-(5-bromo-2-(methylthio)-6-oxopyrimidin-1(6H)-yl)acetate (450 mg, 1.35 mmol) in toluene (10 mL) was added Cs₂CO₃ (1.09 g, 16.02 mmol), BINAP (168 mg, 0.27 mmol) and Pd(OAc)₂ (30.3 mg, 0.13 mmol) at 25° C. under N₂ atmosphere, the reaction mixture was degassed under N₂ atmosphere for three times and stirred at 120° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-29% EtOAc in PE) to give the title compound (700 mg, yield 37.3%) as a yellow oil. LC/MS (ESI) m/z: 466 (M+H)⁺.

### Step 6: Tert-butyl (S)-2-(5-((1-(dibenzo[b,d]furan-2-yl)ethyl)amino)-6-oxo-2-phenylpyrimidin-1(6H)-yl)acetate (8)

To a mixture of tert-butyl (S)-2-(5-((1-(dibenzo[b,d]furan-2-yl)ethyl)amino)-2-(methylthio)-6-oxopyrimidin-1(6H)-yl)acetate (400 mg, 0.86 mmol) and phenylboronic acid (209 mg, 1.71 mmol) in DMF (10 mL) was added CuTC (359 mg, 1.89 mmol), Pd(PPh₃)₄ (99.4 mg, 0.08 mmol) at 25° C. under N₂ atmosphere, the reaction mixture was degassed under N₂ atmosphere for three times and stirred in a sealed tube at 80° C. for 2 days. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-17% EtOAc in PE) to give the title compound (250 mg, yield 58.6%) as a white solid. LC/MS (ESI) m/z: 496 (M+H)⁺.

### Step 7: (S)-2-(5-((1-(dibenzo[b,d]furan-2-yl)ethyl)amino)-6-oxo-2-phenylpyrimidin-1(6H)-yl)-acetic acid (9)

To a solution of tert-butyl (S)-2-(5-((1-(dibenzo[b,d]furan-2-yl)ethyl)amino)-6-oxo-2-phenylpyrimidin-1(6H)-yl)acetate (100 mg, 0.5 mol) in 1,4-dioxane (1 mL) and water (1 mL) was added NaOH (24.2 mg, 0.61 mol), and the mixture was stirred at 90° C. for 5 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (60 mg, yield 27.2%) as a yellow oil, which was used directly in the next step without further purification. LC/MS (ESI) m/z: 440 (M+H)⁺.

### Step 8: (S)—N-((1H-pyrrolo[3,2-c]pyridine-2-yl)methyl)-2-(5-((1-(dibenzo[b,d]furan-2-yl)ethyl)-amino)-6-oxo-2-phenylpyrimidin-1(6H)-yl)acetamide (Compound 127)

To a mixture of (S)-2-(5-((1-(dibenzo-2-yl)ethyl)amino)-6-oxo-2-phenylpyrimidin-1(6H)-yl)acetic acid (60 mg, 0.14 mmol) and (1H-pyrrolo[3,2-c]pyridine-2-yl)methanamine (39.1 mg, 0.26 mmol) in DMF (4 mL) was added DIPEA (52.9 mg, 0.41 mmol) and HATU (62.3 mg, 0.16 mol) and the mixture was stirred at room temperature for 6 hours. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=8:1) and further purified by prep-HPLC to give Compound 127 (22 mg, yield 27.6%) as a white solid. ¹H NMR (400 MHz, DMSO) δ 11.32 (s, 1H), 8.75 (s, 1H), 8.68 (t, J=5.7 Hz, 1H), 8.23 (s, 1H), 8.10 (d, J=8.1 Hz, 2H), 7.70-7.66 (m, 2H), 7.63 (m, 1H), 7.52 (t, J=7.2 Hz, 1H), 7.42-7.37 (m, 4H), 7.35-7.32 (m, 3H), 6.99 (s, 1H), 6.32 (s, 1H), 5.92 (d, J=7.1 Hz, 1H), 4.70-4.64 (m, 1H), 4.54 (d, J=16.2 Hz, 1H), 4.44 (d, J=10.4 Hz, 1H), 4.42 (d, J=5.3 Hz, 2H), 1.61 (d, J=6.7 Hz, 3H). LC/MS (ESI) m/z: 569 (M+H)⁺. RT (Method A): 1.78 min.

| # | Reactant A | Reactant B | Reactant C | Reactant D | Characterization Data |
|---|---|---|---|---|---|
| 117^{a} | 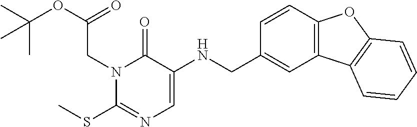 [SMILES: CSc1ncc(NCc2ccc3oc4ccccc4c3c2)c(=O)n1CC(=O)OC(C)(C)C] | | 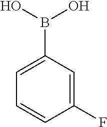 | 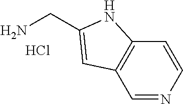 [SMILES: NCc1cc2cnccc2[nH]1 ; Cl] | ¹H NMR (400 MHz, CD₃OD) δ 8.71 (s, 1H), 8.12-8.07 (m, 2H), 8.00 (d, J = 7.4 Hz, 1H), 7.55 (d, J = 3.0 Hz, 2H), 7.48 (s, 1H), 7.45- 7.32 (m, 4H), 7.28 (d, J = 7.5 Hz, 2H), 7.20 (s, 1H), 7.12 (s, 1H), 6.51 (s, 1H), 4.65 (s, 2H), 4.60 (s, 2H), 4.57 (s, 2H). LC/MS (ESI) |
| | | | | | m/z: 573 (M + H)⁺. RT (Method A): |
| | | | | | 1.71 min. |
| 118ª | 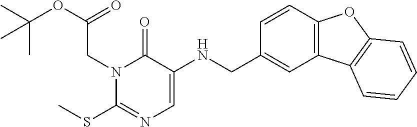 | | 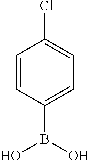 | 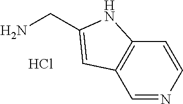 [SMILES: NCc1cc2cnccc2[nH]1 ; Cl] | ¹H NMR (400 MHz, CD₃OD) δ 8.74 (s, 1H), 8.12 (d, J = 5.9 Hz, 1H), 8.08 (s, 1H), 8.00 (d, J = 7.6 Hz, 1H), 7.58-7.55 (m, 2H), 7.48 (s, 1H), 7.43 (d, J = 8.4 Hz, 4H), 7.33 (d, J = 8.5 Hz, 3H), 7.11 (s, 1H), 6.50 (s, 1H), 4.64 (s, 2H), 4.60 (s, 2H), 4.56 (s, 2H). LC/MS (ESI) m/z: 589 (M + H)⁺. RT (Method A): 1.83 min. |
| 119^{a} | 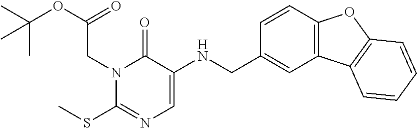 [SMILES: CSc1ncc(NCc2ccc3oc4ccccc4c3c2)c(=O)n1CC(=O)OC(C)(C)C] | | 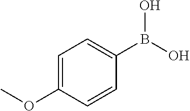 | 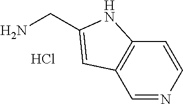 [SMILES: NCc1cc2cnccc2[nH]1 ; Cl] | ¹H NMR (400 MHz, DMSO) δ 11.32 (s, 1H), 8.70 (dd, J = 15.0, 9.6 Hz, 2H), 8.18-8.09 (m, 3H), 7.72-7.65 (m, 2H), 7.60-7.49 (m, 2H), 7.39 (t, J = 7.5 Hz, 1H), 7.34 (d, J = 8.8 Hz, 3H), 7.07 (d, J = 15.5 Hz, 1H), 6.85 (d, J = 8.7 Hz, 2H), 6.29 (m, 2H), 4.50 (d, J = |
| | | | | | 5.7 Hz, 4H), 4.43 (d, J = 5.5 Hz, |
| | | | | | 2H), 3.71 (s, 3H). LC/MS (ESI) |
| | | | | | m/z: 585 (M + H)⁺. RT (Method |
| | | | | | A): 1.68 min. |
| 120^{a} | 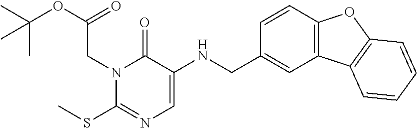 [SMILES: CSc1ncc(NCc2ccc3oc4ccccc4c3c2)c(=O)n1CC(=O)OC(C)(C)C] | | 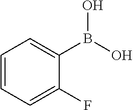 | 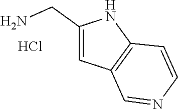 [SMILES: NCc1cc2cnccc2[nH]1 ; Cl] | ¹H NMR (400 MHz, CD₃OD) δ 8.79 (s, 1H), 8.15 (d, J = 6.0 Hz, 1H), 8.08 (s, 1H), 8.00 (d, J = 7.9 Hz, 1H), 7.59-7.53 (m, 3H), 7.53- 7.50 (m, 1H), 7.50-7.45 (m, 2H), 7.44-7.38 (m, 1H), 7.38- 7.34 (m, 1H), 7.20 (t, J = 9.1 Hz, 1H), 7.15-7.09 (m, 2H), 6.55 (s, |
| | | | | | 1H), 4.85-4.83 (m, 2H), 4.62- |
| | | | | | 4.58 (m, 2H), 4.50 (s, 2H). LC/MS |
| | | | | | (ESI) m/z: 573.4 (M + H)⁺. RT |
| | | | | | (Method A): 1.68 min. |
| 126^{b} | 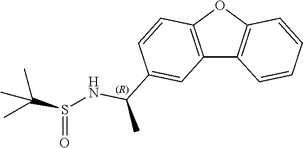 | 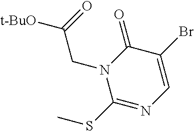 | 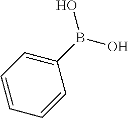 | 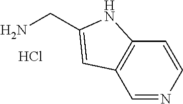 [SMILES: NCc1cc2cnccc2[nH]1 ; Cl] | ¹H NMR (400 MHz, CD₃OD) δ 8.72 (s, 1H), 8.11 (d, J = 5.7 Hz, 1H), 8.08 (s, 1H), 8.00 (d, J = 7.7 Hz, 1H), 7.57-7.51 (m, 3H), 7.49- 7.44 (m, 1H), 7.41-7.37 (m, 4H), 7.33 (d, J = 7.5 Hz, 3H), 6.92 (s, 1H), 6.49 (s, 1H), 4.70-4.65 (m, 1H), 4.62 (d, J = 3.0 Hz, 2H), 4.55 (s, 2H), 1.68 (d, J = 6.7 Hz, 3H). LC/MS (ESI) m/z: 569 |
| | | | | | (M + H)⁺. RT (Method A): 1.76 min. |
| 134^{a} | 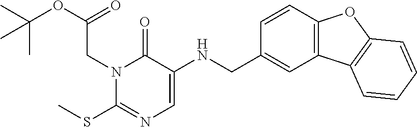 [SMILES: CSc1ncc(NCc2ccc3oc4ccccc4c3c2)c(=O)n1CC(=O)OC(C)(C)C] | | 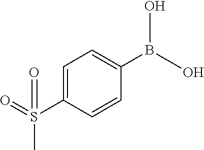 | 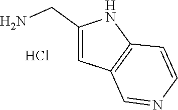 [SMILES: NCc1cc2cnccc2[nH]1 ; Cl] | ¹H NMR (400 MHz, DMSO) δ 11.33 (s, 1H), 8.75 (s, 2H), 8.18 (s, 1H), 8.12 (d, J = 5.8 Hz, 2H), 7.92 (d, J = 8.4 Hz, 2H), 7.71- 7.69 (m, 2H), 7.68 (d, J = 5.9 Hz, 1H), 7.60-7.57 (m, 1H), 7.54- 7.50 (m, 1H), 7.39 (dd, J = 11.1, 4.0 Hz, 1H), 7.33 (d, J = 5.6 Hz, 1H), 7.14 (s, 1H), 6.54-6.47 (m, 1H), 6.34-6.33 (m, 1H), 5.33 (dd, |
| | | | | | J = 7.0, 2.5 Hz, 1H), 4.52 (s, 4H), |
| | | | | | 4.43 (d, J = 5.5 Hz, 2H), 3.22 (s, |
| | | | | | 3H). LC/MS (ESI) m/z: 633 |
| | | | | | (M + H)⁺. RT (Method A): 1.57 min. |
| 135^{a} | 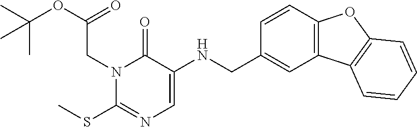 [SMILES: CSc1ncc(NCc2ccc3oc4ccccc4c3c2)c(=O)n1CC(=O)OC(C)(C)C] | | 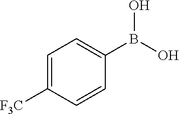 [SMILES: OB(O)c1ccc(C(F)(F)F)cc1] | 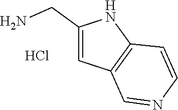 [SMILES: NCc1cc2cnccc2[nH]1 ; Cl] | ¹H NMR (400 MHz, DMSO-d₆) δ 11.30 (s, 1H), 8.76-8.61 (m, 2H), 8.19-8.08 (m, 3H), 7.71-7.61 (m, 6H), 7.57 (m, 1H), 7.53-7.48 (m, 1H), 7.38 (t, J = 7.5 Hz, 1H), 7.32 (d, J = 5.7 Hz, 1H), 7.11 (m, 1H), 6.49 (t, J = 6.4 Hz, 1H), 6.31 (s, 1H), 4.51 (d, J = 5.9 Hz, 4H), |
| | | | | | 4.41 (d, J = 5.5 Hz, 2H). LC/MS |
| | | | | | (ESI) m/z: 623 (M + H)⁺. RT |
| | | | | | (Method A): 1.94 min. |
| 136ª | 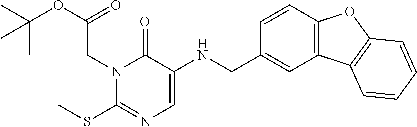 [SMILES: CSc1ncc(NCc2ccc3oc4ccccc4c3c2)c(=O)n1CC(=O)OC(C)(C)C] | | 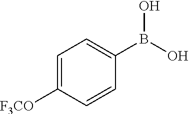 [SMILES: OB(O)c1ccc(OC(F)(F)F)cc1] | 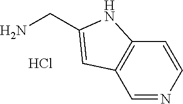 [SMILES: NCc1cc2cnccc2[nH]1 ; Cl] | ¹H NMR (400 MHz, CD₃OD) δ 8.72-8.71 (m, 1H), 8.11 (d, J = 5.9 Hz, 1H), 8.08-8.06 (m, 1H), 7.99 (d, 1H), 7.57-7.53 (m, 5H), 7.49-7.45 (m, 1H), 7.42-7.41 (m, 1H), 7.35 (t, J = 7.0 Hz, 1H), 7.23 (d, J = 8.1 Hz, 2H), 7.11 (s, 1H), 6.51 (s, 1H), 4.63 (s, 2H), |
| | | | | | 4.60 (s, 2H), 4.56 (s, 2H). LC/MS |
| | | | | | (ESI) m/z: 639 (M + H)⁺. RT |
| | | | | | (Method A): 1.90 min. |
| 155^{a,e} | 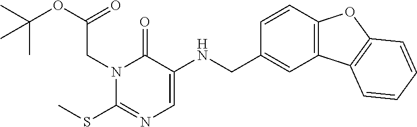 [SMILES: CSc1ncc(NCc2ccc3oc4ccccc4c3c2)c(=O)n1CC(=O)OC(C)(C)C] | | 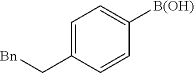 | 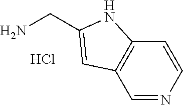 [SMILES: NCc1cc2cnccc2[nH]1 ; Cl] | ¹H NMR (400 MHz, DMSO-d₆) δ 11.32 (s, 1H), 8.75 (s, 1H), 8.70 (t, J = 5.6 Hz, 1H), 8.17 (s, 1H), 8.13 (s, 1H), 8.11 (d, J = 1.5 Hz, 1H), 7.71-7.66 (m, 2H), 7.57 (dd, J = 8.5, 1.6 Hz, 1H), 7.51 (dd, J = 11.3, 4.2 Hz, 1H), 7.43-7.39 (m, 5H), 7.36 (d, J = 4.1 Hz, 2H), 7.34 |
| | | | | | (d, J = 1.6 Hz, 2H), 7.09 (s, 1H), |
| | | | | | 6.95 (s, 1H), 6.93 (s, 1H), 6.36 (s, |
| | | | | | 1H), 6.28 (t, J = 6.3 Hz, 1H), 5.03 |
| | | | | | (s, 2H), 4.50 (d, J = 6.8 Hz, 4H), |
| | | | | | 4.43 (d, J = 5.2 Hz, 2H). LC/MS |
| | | | | | (ESI) m/z: 661 (M + H)⁺. RT |
| | | | | | (Method A): 2.05 min. |
| 226ª | 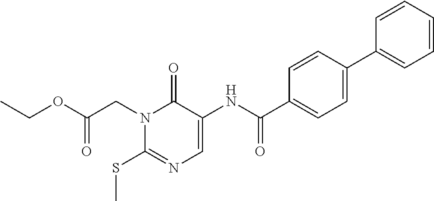 | | 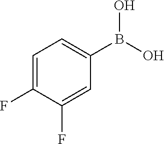 [SMILES: OB(O)c1ccc(F)c(F)c1] | 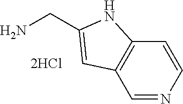 [SMILES: NCc1cc2cnccc2[nH]1 ; "2HCl"] | ¹H NMR (400 MHz, DMSO-d₆) δ 11.35 (1H, s), 9.58 (1H, s), 8.79- 8.84 (2H, m), 8.08 (2H, d, J = 8.0 Hz), 7.86 (2H, d, J = 8.0 Hz), 7.77 (2H, d, J = 7.3 Hz), 7.71 (2H, m), 7.49-7.56 (4H, m), 7.40-7.46 (3H, m), 6.33 (1H, s), 4.65 (2H, s) and 4.43 (2H, d, J = 5.4 Hz). LC/MS (ESI) m/z: 591 (M + H)⁺. RT (Method A): 1.68 min. |
| 229^{c,d} | 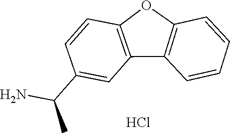 [SMILES: C[C@@H](N)c1ccc2oc3ccccc3c2c1 ; Cl] | 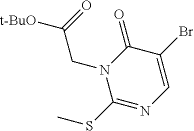 [SMILES: CSc1ncc(Br)c(=O)n1CC(=O)OC(C)(C)C] | 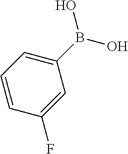 | 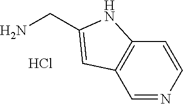 [SMILES: NCc1cc2cnccc2[nH]1 ; Cl] | ¹H NMR (400 MHz, CD₃OD) δ 8.70 (s, 1H), 8.10 (d, J = 5.8 Hz, 1H), 8.06 (s, 1H), 7.98 (d, J = 7.6 Hz, 1H), 7.56-7.52 (m, 3H), 7.46 (m, 1H), 7.38 (d, J = 5.8 Hz, 1H), 7.35-7.30 (m, 2H), 7.21 (d, J = 6.7 Hz, 1H), 7.20-7.13 (m, 2H), 6.92 (s, 1H), 6.49 (s, 1H), 4.67 (d, J = 6.5 Hz, 1H), 4.62 (d, J = 5.9 Hz, 2H), 4.55 (d, J = 1.1 Hz, 2H), |
| | | | | | 1.66 (d, J = 6.7 Hz, 3H). LC/MS |
| | | | | | (ESI) m/z: 587 (M + H)⁺. RT |
| | | | | | (Method A): 1.80 min. |
| 230^{a} | 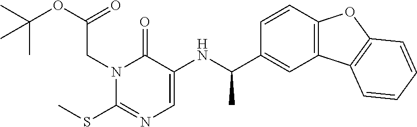 [SMILES: CSc1ncc(N[C@H](C)c2ccc3oc4ccccc4c3c2)c(=O)n1CC(=O)OC(C)(C)C] | | 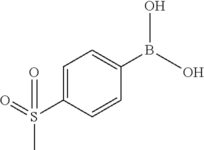 | 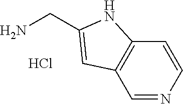 [SMILES: NCc1cc2cnccc2[nH]1 ; Cl] | ¹H NMR (400 MHz, DMSO-d₆) δ 11.34 (s, 1H), 8.76 (s, 2H), 8.23 (s, 1H), 8.13-8.09 (m, 2H), 7.91 (d, J = 8.3 Hz, 2H), 7.69 (d, J = 7.7 Hz, 2H), 7.65 (d, J = 6.6 Hz, 3H), 7.51 (d, J = 7.9 Hz, 1H), 7.40 (t, J = 7.4 Hz, 1H), 7.32 (d, J = 5.7 Hz, 1H), 7.02 (s, 1H), 6.33 (s, 1H), 6.09 (d, J = 7.1 Hz, 1H), 4.72- 4.66 (m, 1H), 4.56 (d, J = 16.3 Hz, |
| | | | | | 1H), 4.47-4.41 (m, 3H), 3.21 (s, |
| | | | | | 3H), 1.61 (d, J = 6.7 Hz, 3H). |
| | | | | | LC/MS (ESI) (m/z): 647 (M + H)⁺. |
| | | | | | RT (Method A): 1.64 min. |
| 232^{a,c} | 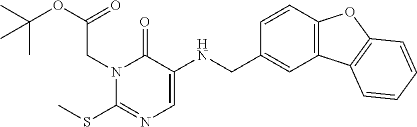 [SMILES: CSc1ncc(NCc2ccc3oc4ccccc4c3c2)c(=O)n1CC(=O)OC(C)(C)C] | | 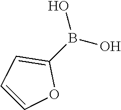 | 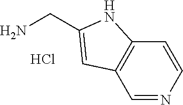 [SMILES: NCc1cc2cnccc2[nH]1 ; Cl] | ¹H NMR (400 MHz, DMSO-d₆) δ 11.35 (s, 1H), 8.80-8.76 (m, 1H), 8.74 (s, 1H), 8.17-8.15 (m, 1H), 8.12-8.10 (m, 2H), 7.68 (dd, J = 9.1, 4.7 Hz, 3H), 7.53 (m, 2H), 7.39 (t, J = 7.2 Hz, 1H), 7.32 (d, J = 5.7 Hz, 1H), 7.12 (s, 1H), 6.78 (d, J = 3.4 Hz, 1H), 6.55 (d, J = |
| | | | | | 3.4, 1.7 Hz, 1H), 6.51 (t, J = 6.4 |
| | | | | | Hz, 1H), 6.37 (s, 1H), 4.87 (s, 2H), |
| | | | | | 4.51 (d, J = 6.4 Hz, 2H), 4.45 (d, |
| | | | | | J = 5.5 Hz, 2H). LC/MS (ESI) m/z: |
| | | | | | 545 (M + H)⁺. RT (Method A): |
| | | | | | 1.56 min. |
| 237^{a,d} | 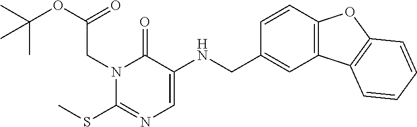 [SMILES: CSc1ncc(NCc2ccc3oc4ccccc4c3c2)c(=O)n1CC(=O)OC(C)(C)C] | | 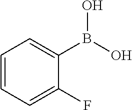 | 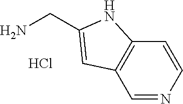 [SMILES: NCc1cc2cnccc2[nH]1 ; Cl] | ¹H NMR (400 MHz, DMSO-d₆) δ 12.44 (s, 1H), 9.14 (s, 1H), 8.83 (t, J = 5.6 Hz, 1H), 8.35 (d, J = 6.5 Hz, 1H), 7.85 (d, J = 6.4 Hz, 1H), 7.67-7.63 (m, 4H), 7.49-7.41 (m, 7H), 7.38-7.33 (m, 3H), 7.05 (s, 1H), 6.71 (s, 1H), 6.28 (t, J = 6.4 Hz, 1H), 4.55-4.49 (m, 4H), |
| | | | | | 4.40 (d, J = 6.4 Hz, 2H). LC/MS |
| | | | | | (ESI) m/z: 587 (M + H)⁺. RT |
| | | | | | (Method A): 1.76 min. |
| 238^{a,d} | 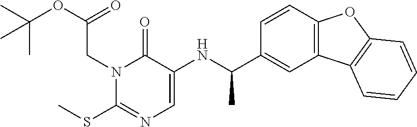 [SMILES: CSc1ncc(N[C@H](C)c2ccc3oc4ccccc4c3c2)c(=O)n1CC(=O)OC(C)(C)C] | | 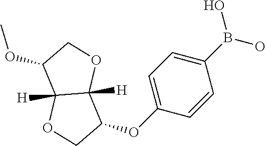 [SMILES: CO[C@@H]1CO[C@H]2[C@@H]1OC[C@H]2Oc1ccc(B(O)O)cc1] | 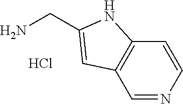 [SMILES: NCc1cc2cnccc2[nH]1 ; Cl] | ¹H NMR (400 MHz, DMSO-d₆) δ 11.34 (s, 1H), 8.76 (s, 1H), 8.70 (t, J = 5.5 Hz, 1H), 8.22 (s, 1H), 8.15- 8.11 (m, 2H), 8.10 (d, J = 7.6 Hz, 1H), 7.70-7.65 (m, 2H), 7.62 (M, 1H), 7.51 (t, J = 7.2 Hz, 1H), 7.40 (d, J = 7.4 Hz, 1H), 7.35 (d, J = 5.6 Hz, 1H), 7.29 (d, J = 8.8 Hz, 2H), 6.97 (s, 1H), 6.90 (d, J = |
| | | | | | 8.8 Hz, 2H), 6.37 (s, 1H), 5.86 (d, |
| | | | | | J = 7.1 Hz, 1H), 4.77 (d, J = 5.4 |
| | | | | | Hz, 1H), 4.69 (s, 1H), 4.65 (d, J = |
| | | | | | 6.7 Hz, 1H), 4.57 (d, J = 4.9 Hz, |
| | | | | | 1H), 4.48 (d, J = 14.2 Hz, 1H), |
| | | | | | 4.43 (d, J = 5.4 Hz, 2H), 4.03- |
| | | | | | 3.99 (m, 1H), 3.89-3.86 (m, 1H), |
| | | | | | 3.79 (d, J = 6.7 Hz, 1H), 3.72 (d, |
| | | | | | J = 2.6 Hz, 1H), 3.42 (s, 3H), |
| | | | | | 1.60 (d, J = 6.7 Hz, 3H). LC/MS |
| | | | | | (ESI) m/z: 727 (M + H)⁺. |
| | | | | | RT (Method A): 1.67 min. |
| 241^{a,d} | 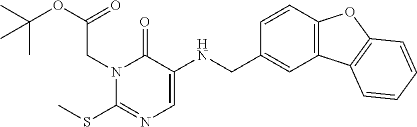 [SMILES: CSc1ncc(NCc2ccc3oc4ccccc4c3c2)c(=O)n1CC(=O)OC(C)(C)C] | | 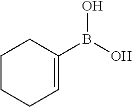 | 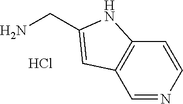 [SMILES: NCc1cc2cnccc2[nH]1 ; Cl] | ¹H NMR (400 MHz, DMSO-d₆) δ 11.31 (s, 1H), 8.80-8.72 (m, 2H), 8.15-8.09 (m, 3H), 7.70-7.65 (m, 2H), 7.56-7.43 (m, 2H), 7.41- 7.32 (m, 2H), 7.01-6.92 (m, 1H), 6.42 (s, 1H), 6.24-6.10 (m, 1H), 5.81-5.76 (m, 1H), 4.67 (s, 2H), 4.49-4.43 (m, 4H), 2.10- |
| | | | | | 2.04 (m, 2H), 1.98-1.92 (m, 2H), |
| | | | | | 1.55-1.45 (m, 4H). LC/MS (ESI) |
| | | | | | (m/z): 559 (M + H)⁺. RT (Method |
| | | | | | A): 1.68 min. |
| 242^{a,d} | 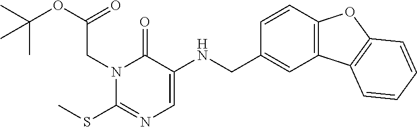 [SMILES: CSc1ncc(NCc2ccc3oc4ccccc4c3c2)c(=O)n1CC(=O)OC(C)(C)C] | | 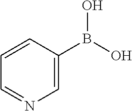 | 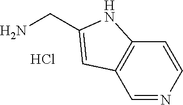 [SMILES: NCc1cc2cnccc2[nH]1 ; Cl] | ¹H NMR (400 MHz, DMSO-d₆) δ 11.32 (s, 1H), 8.77-8.71 (m, 2H), 8.64-8.59 (m, 2H), 8.22-8.14 (m, 1H), 8.13-8.09 (m, 2H), 7.86- 7.82 (m, 1H), 7.72-7.66 (m, 2H), 7.59-7.45 (m, 2H), 7.41- 7.35 (m, 2H), 7.34-7.30 (m, 1H), 7.15-7.10 (m, 1H), 6.55-6.45 |
| | | | | | (m, 1H), 6.31 (s, 1H), 4.57-4.49 |
| | | | | | (m, 4H), 4.43-4.38 (m, 2H). |
| | | | | | LC/MS (ESI) (m/z): 556 (M + H)⁺. |
| | | | | | RT (Method A): 1.42 min. |
| 248^{a,d} | 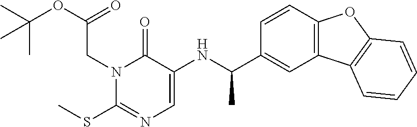 [SMILES: CSc1ncc(N[C@H](C)c2ccc3oc4ccccc4c3c2)c(=O)n1CC(=O)OC(C)(C)C] | | 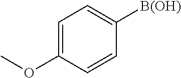 | 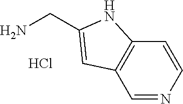 [SMILES: NCc1cc2cnccc2[nH]1 ; Cl] | ¹H NMR (400 MHz, DMSO-d₆) δ 11.33 (s, 1H), 8.74 (s, 1H), 8.69 (t, J = 5.6 Hz, 1H), 8.22 (d, J = 1.3 Hz, 1H), 8.12 (d, J = 5.7 Hz, 1H), 8.09 (s, 1H), 7.70 - 7.65 (m, 2H), 7.62 (dd, J = 8.5, 1.7 Hz, 1H), 7.50 (d, J = 7.2 Hz, 1H), 7.40 (t, J = 5.7 Hz, 1H), 7.34 (d, J = 5.7 |
| | | | | | Hz, 1H), 7.32 (s, 1H), 7.30 (s, 1H), |
| | | | | | 6.97 (s, 1H), 6.84 (s, 1H), 6.82 (s, |
| | | | | | 1H), 6.35 (s, 1H), 5.86 (d, J = 7.1 |
| | | | | | Hz, 1H), 4.68-4.63 (m, 1H), 4.54 |
| | | | | | (d, J = 16.3 Hz, 1H), 4.46-4.41 |
| | | | | | (m, 3H), 3.70 (s, 3H), 1.60 (d, J = |
| | | | | | 6.7 Hz, 3H). LC/MS (ESI) m/z: |
| | | | | | 599 (M + H)⁺. RT (Method A): |
| | | | | | 1.74 min. |
| 251^{a,d} | 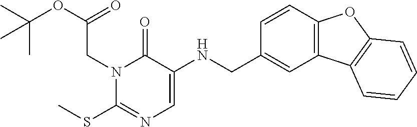 [SMILES: CSc1ncc(NCc2ccc3oc4ccccc4c3c2)c(=O)n1CC(=O)OC(C)(C)C] | | 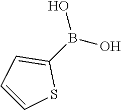 | 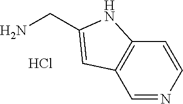 [SMILES: NCc1cc2cnccc2[nH]1 ; Cl] | ¹H NMR (400 MHz, DMSO-d₆) δ 11.46 (s, 1H), 8.91-8.85 (m, 1H), 8.79-8.75 (m, 1H), 8.17-8.16 (m, 1H), 8.15-8.10 (m, 2H), 7.72- 7.65 (m, 2H), 7.65-7.62 (m, 1H), 7.60-7.53 (m, 1H), 7.53- 7.49 (m, 1H), 7.41-7.36 (m, 2H), 7.23-7.20 (m, 1H), 7.14-7.07 |
| | | | | | (m, 1H), 7.02-6.99 (m, 1H), 6.51- |
| | | | | | 6.43 (m, 1H), 6.44-6.40 (m, |
| | | | | | 1H), 4.79 (s, 2H), 4.53-4.48 (m, |
| | | | | | 4H). LC/MS (ESI) m/z: 561 |
| | | | | | (M + H)⁺. RT (Method A): 1.65 min. |
| 252^{a,d} | 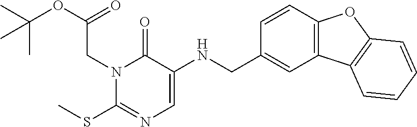 [SMILES: CSc1ncc(NCc2ccc3oc4ccccc4c3c2)c(=O)n1CC(=O)OC(C)(C)C] | | 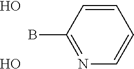 | 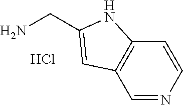 [SMILES: NCc1cc2cnccc2[nH]1 ; Cl] | ¹H NMR (400 MHz, DMSO-d₆) δ 11.23 (s, 1H), 8.71 (s, 1H), 8.59 (t, J = 5.8 Hz, 1H), 8.48 (d, J = 4.1 Hz, 1H), 8.18 - 8.09 (m, 3H), 7.85- 7.79 (m, 1H), 7.73-7.66 (m, 3H), 7.60-7.47 (m, 2H), 7.39 (t, J = 6.3 Hz, 2H), 7.28 (d, J = 5.6 Hz, 1H), 7.16-7.11 (m, 1H), 6.65- |
| | | | | | 6.51 (m, 1H), 6.18 (s, 1H), 5.18 (s, |
| | | | | | 2H), 4.57-4.50 (m, 2H), 4.32 (d, |
| | | | | | J = 5.4 Hz, 2H). LC/MS (ESI) |
| | | | | | (m/z): 556 (M + H)⁺. RT (Method |
| | | | | | A): 1.52 min. |
| 272^{a,d} | 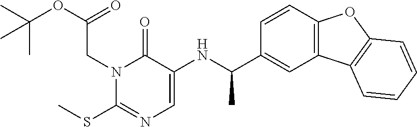 [SMILES: CSc1ncc(N[C@H](C)c2ccc3oc4ccccc4c3c2)c(=O)n1CC(=O)OC(C)(C)C] | | 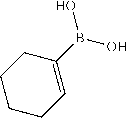 | 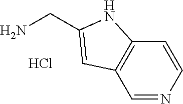 [SMILES: NCc1cc2cnccc2[nH]1 ; Cl] | ¹H NMR (400 MHz, DMSO-d₆) δ 11.31 (s, 1H), 8.77-8.72 (m, 2H), 8.19 (d, J = 1.3 Hz, 1H), 8.10 (t, J = 6.3 Hz, 2H), 7.68 (d, J = 8.2 Hz, 1H), 7.65 (d, J = 8.5 Hz, 1H), 7.59 (dd, J = 8.5, 1.7 Hz, 1H), 7.53-7.49 (m, 1H), 7.39 (t, J = 7.5 Hz, 1H), 7.32 (d, J = |
| | | | | | 5.7 Hz, 1H), 6.86 |
| | | | | | (s, 1H), 6.41 (s, 1H), 5.77-5.71 |
| | | | | | (m, 2H), 4.69 (d, J = 16.2 Hz, 1H), |
| | | | | | 4.61 (dd, J = 14.5, 7.0 Hz, 2H), |
| | | | | | 4.45 (d, J = 5.6 Hz, 2H), 2.04 (s, |
| | | | | | 2H), 1.93 (s, 2H), 1.58 (d, J = 6.7 |
| | | | | | Hz, 3H), 1.52-1.43 (m, 4H). |
| | | | | | LC/MS (ESI) m/z: 573 (M + H)⁺. |
| | | | | | RT (Method A): 1.76 min. |
| 273^{a,d} | 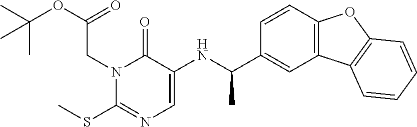 [SMILES: CSc1ncc(N[C@H](C)c2ccc3oc4ccccc4c3c2)c(=O)n1CC(=O)OC(C)(C)C] | | 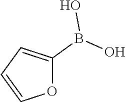 | 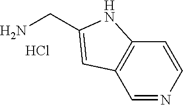 [SMILES: NCc1cc2cnccc2[nH]1 ; Cl] | ¹H NMR (400 MHz, CD₃OD) δ 8.73 (s, 1H), 8.14-8.10 (m, 1H), 8.06 (s, 1H), 8.00-7.97 (m, 1H), 7.55-7.53 (m, 2H), 7.48-7.44 (m, 2H), 7.41 (d, J = 5.9 Hz, 1H), 7.36-7.31 (m, 1H), 6.92 (s, 1H), 6.78 (d, J = 3.3 Hz, 1H), 6.56 (s, 1H), 6.46-6.41 (m, 1H), 4.96 (s, |
| | | | | | 2H), 4.71-4.66 (m, 1H), 4.60 (s, |
| | | | | | 2H), 1.67 (d, J = 6.7 Hz, 3H). |
| | | | | | LC/MS (ESI) m/z: 559 (M + H)⁺. |
| | | | | | RT (Method A): 1.73 min. |
| 274a,d | 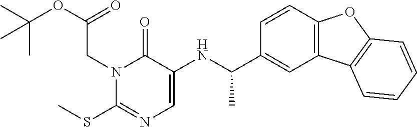 [SMILES: CSc1ncc(N[C@@H](C)c2ccc3oc4ccccc4c3c2)c(=O)n1CC(=O)OC(C)(C)C] | | 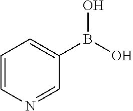 | 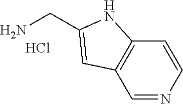 [SMILES: NCc1cc2cnccc2[nH]1 ; Cl] | ¹H NMR (400 MHz, CD₃OD) δ 8.72 (s, 1H), 8.64 (s, 1H), 8.57 (d, J = 3.9 Hz, 1H), 8.12 (d, J = 5.8 Hz, 1H), 8.08 (s, 1H), 7.99 (d, J = 7.7 Hz, 1H), 7.89 (d, J = 7.9 Hz, 1H), 7.56 (d, J = 9.6 Hz, 3H), 7.47 (t, J = 7.6 Hz, 1H), 7.40 (d, J = 6.0 Hz, 1H), 7.38-7.31 (m, 2H), 6.97 |
| | | | | | (s, 1H), 6.50 (s, 1H), 4.71 (d, J = |
| | | | | | 6.5 Hz, 1H), 4.65 (d, J = 6.0 Hz, |
| | | | | | 2H), 4.56 (s, 2H), 1.68 (d, J = 6.7 |
| | | | | | Hz, 3H). LC/MS (ESI) m/z: 570 |
| | | | | | (M + H)⁺. RT (Method A): 1.53 min. |
| 279^{a} | 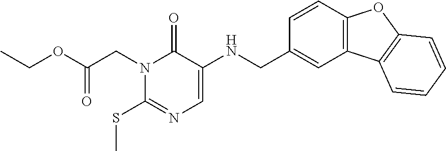 [SMILES: CCOC(=O)Cn1c(SC)ncc(NCc2ccc3oc4ccccc4c3c2)c1=O] | | 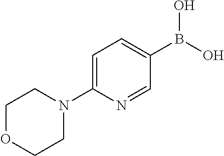 [SMILES: OB(O)c1ccc(N2CCOCC2)nc1] | 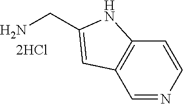 [SMILES: "2HCl" ; NCc1cc2cnccc2[nH]1] | ¹H NMR (400 MHz, DMSO-d₆) δ 11.33 (1H, s), 8.71-8.76 (2H, m), 8.17 (2H, m), 8.09-8.14 (2H, m), 7.67 (2H, t, J = 7.9 Hz), 7.57 (2H, m), 7.51 (1H, t, J = 7.9 Hz), 7.39 (1H, t, J = 7.5 Hz), 7.34 (1H, d, J = 5.7 Hz), 7.10 (1H, s), 6.67 (1H, d, J = 9.0 Hz), 6.34 (1H, s), 6.27 (1H, t, J = 6.4 Hz), 4.55 (2H, s), 4.49 (2H, d, J = 6.3 Hz), 4.44 (2H, |
| | | | | | d, J = 5.6 Hz), 3.63-3.67 (4H, m) |
| | | | | | and 3.40-3.47 (4H, m). LC/MS |
| | | | | | (ESI) m/z: 641.33 (M + H)⁺. RT |
| | | | | | (Method A): 1.56 min. |
| 280^{a} | 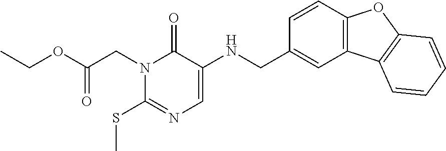 [SMILES: CCOC(=O)Cn1c(SC)ncc(NCc2ccc3oc4ccccc4c3c2)c1=O] | | 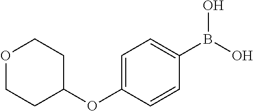 [SMILES: OB(O)c1ccc(OC2CCOCC2)cc1] | 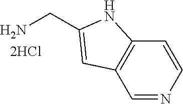 [SMILES: "2HCl" ; NCc1cc2cnccc2[nH]1] | ¹H NMR (400 MHz, DMSO-d₆) δ 11.95 (1H, s), 8.94 (1H, s), 8.79 (1H, t, J = 6.8 Hz), 8.24 (1H, d, J = 6.1 Hz), 8.16 (1H, s), 8.11 (1H, d, J = 7.6 Hz), 7.68 (2H, m), 7.59 (2H, m), 7.52 (1H, t, J = 7.6 Hz), 7.39 (1H, t, J = 7.5 Hz), 7.32 (2H, d, J = 8.4 Hz), 7.09 (1H, s), 6.89 (2H, d, J = 6.7 Hz), 6.55 (1H, s), 6.23 (1H, t, J = 6.5 Hz), 4.49-4.55 |
| | | | | | (7H, m), 3.83 (2H, m), 3.45 (2H, |
| | | | | | m), 1.93 (2H, m) and 1.55 (2H, |
| | | | | | m). LC/MS (ESI) m/z: 655.40 |
| | | | | | (M + H)⁺. RT (Method A): 1.70 min. |
| 283^{a,d} | 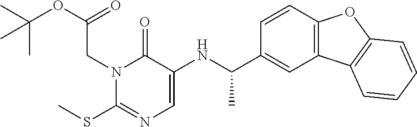 [SMILES: CSc1ncc(N[C@@H](C)c2ccc3oc4ccccc4c3c2)c(=O)n1CC(=O)OC(C)(C)C] | | 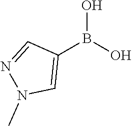 | 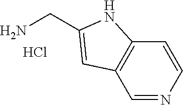 [SMILES: NCc1cc2cnccc2[nH]1 ; Cl] | ¹H NMR (400 MHz, DMSO-d₆) δ 11.44 (s, 1H), 8.84 (t, J = 5.4 Hz, 1H), 8.77 (s, 1H), 8.20 (s, 1H), 8.15-8.09 (m, 2H), 7.76 (d, J = 6.8 Hz, 1H), 7.69-7.64 (m, 2H), 7.60 (d, J = 8.1 Hz, 1H), 7.53-7.49 (m, 2H), 7.41-7.35 (m, 2H), 6.93 (d, J= 8.5 Hz, 1H), |
| | | | | | 6.42 (s, 1H), 5.78 (d, J = |
| | | | | | 7.2 Hz, 1H), 4.77 (d, J = 16.7 Hz, |
| | | | | | 1H), 4.69-4.62 (m, 2H), 4.48 (d, J = |
| | | | | | 5.3 Hz, 2H), 3.74 (s, 3H), 1.59 (d, |
| | | | | | J = 6.7 Hz, 3H). LCMS (ESI) |
| | | | | | (m/z): 573 (M + H)⁺. |
| 287^{a,d} | 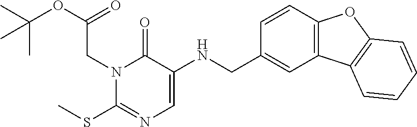 [SMILES: CSc1ncc(NCc2ccc3oc4ccccc4c3c2)c(=O)n1CC(=O)OC(C)(C)C] | | 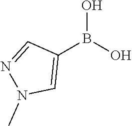 | 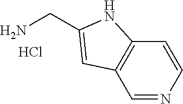 [SMILES: NCc1cc2cnccc2[nH]1 ; Cl] | ¹H NMR (400 MHz, DMSO-d₆) δ 11.37 (s, 1H), 8.84 (t, J = 5.6 Hz, 1H), 8.74 (s, 1H), 8.17-8.08 (m, 3H), 7.80 (s, 1H), 7.71-7.63 (m, 2H), 7.58-7.47 (m, 3H), 7.41- 7.32 (m, 2H), 7.05 (d, J = 14.8 Hz, 1H), 6.40 (s, 1H), 6.30-6.18 (m, 1H), 4.73 (s, 2H), 4.52-4.46 (m, |
| | | | | | 4H), 3.76 (s, 3H). LC/MS (ESI) |
| | | | | | m/z: 559 (M + H)⁺. RT (Method |
| | | | | | A): 1.36 min. |
| 292^{c} | 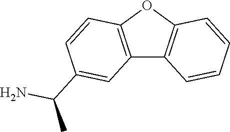 [SMILES: C[C@@H](N)c1ccc2oc3ccccc3c2c1] | 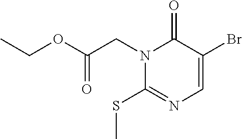 [SMILES: CCOC(=O)Cn1c(SC)ncc(Br)c1=O] | 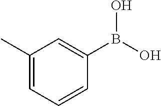 | 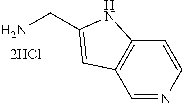 [SMILES: "2HCl" ; NCc1cc2cnccc2[nH]1] | ¹H NMR (400 MHz, DMSO-d₆) δ 11.31 (1H, s), 8.74 (1H, s), 8.66 (1H, t, J = 6.7 Hz), 8.11 (2H, m), 7.61-7.69 (3H, m), 7.52 (1H, t, J = 7.6 Hz), 7.39 (1H, t, J = 7.5 Hz), 7.32 (1H, d, J = 5.6 Hz), 7.22 (2H, m), 7.17 (2H, m), 6.98 (1H, s), 6.31 (1H, s), 5.86 (1H, d, J = 7.0 Hz), 4.67 (1H, quint, J = 6.7 Hz), |
| | | | | | 4.54 (1H, d, J = 16.3 Hz), 4.32 |
| | | | | | (3H, m), 2.19 (3H, s) and 1.61 |
| | | | | | (3H, d, J = 6.7 Hz). LC/MS (ESI) |
| | | | | | m/z: 583.14 (M + H)⁺. RT (Method |
| | | | | | A): 1.78 min. |
| 293^{a,f} | 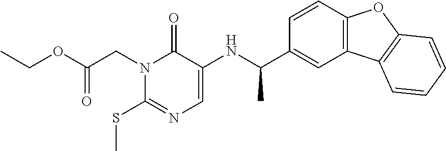 [SMILES: CCOC(=O)Cn1c(SC)ncc(N[C@H](C)c2ccc3oc4ccccc4c3c2)c1=O] | | 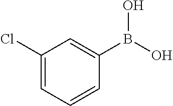 | 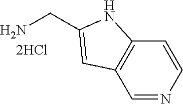 [SMILES: "2HCl" ; NCc1cc2cnccc2[nH]1] | ¹H NMR (400 MHz, DMSO-d₆) δ 11.34 (1H, s), 8.75 (1H, s), 8.72 (1H, t, J = 5.7 Hz), 8.22 (1H, s), 8.12 (2H, m), 7.60-7.69 (3H, m), 7.45-7.53 (3H, m), 7.32-7.40 (4H, m), 7.00 (1H, s), 6.32 (1H, s), 5.97 (1H, d, J = 7.1 Hz), 4.68 (1H, quint, J = 6.8 Hz), 4.57 (1H, d, J = 16.3 Hz), 4.42-4.46 (3H, m) and 1.61 (3H, d, J = 6.7 Hz). LC/MS |
| | | | | | (ESI) m/z: 603.31 (M + H)⁺. RT |
| | | | | | (Method A): 1.86 min. |
| 296^{a,d} | 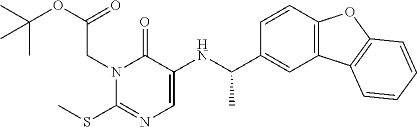 [SMILES: CSc1ncc(N[C@@H](C)c2ccc3oc4ccccc4c3c2)c(=O)n1CC(=O)OC(C)(C)C] | | 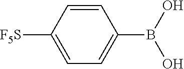 | 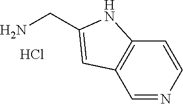 [SMILES: NCc1cc2cnccc2[nH]1 ; Cl] | ¹H NMR (400 MHz, CD₃OD) δ 8.72 (s, 1H), 8.12 (d, J = 5.8 Hz, 1H), 8.07 (s, 1H), 7.99 (d, J = 7.7 Hz, 1H), 7.72 (d, J = 8.5 Hz, 2H), 7.60-7.53 (m, 5H), 7.46 (t, J = 7.7 Hz, 1H), 7.40 (d, J = 5.9 Hz, 1H), 7.34 (t, J = 7.5 Hz, 1H), 6.94 (s, 1H), 6.51 (s, 1H), 4.70 (d, J = |
| | | | | | 6.7 Hz, 1H), 4.60 (d, J = 5.5 Hz, |
| | | | | | 2H), 4.55 (s, 2H), 1.68 (d, J = 6.7 |
| | | | | | Hz, 3H). LC/MS (ESI) m/z: 695 |
| | | | | | (M + H)⁺. RT (Method A): 2.07 min. |
| 297^{a,d} | 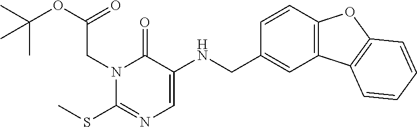 [SMILES: CSc1ncc(NCc2ccc3oc4ccccc4c3c2)c(=O)n1CC(=O)OC(C)(C)C] | | 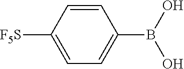 | 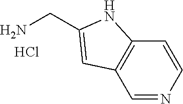 [SMILES: Cl ; NCc1cc2cnccc2[nH]1] | ¹H NMR (400 MHz, CD₃OD) δ 9.00 (d, J = 6.5 Hz, 1H), 8.29 (t, J = 6.3 Hz, 1H), 8.06 (s, 1H), 7.98 (d, J = 7.5 Hz, 1H), 7.88 (t, J = 6.1 Hz, 1H), 7.83 (d, J = 8.4 Hz, 2H), 7.70-7.61 (m, 3H), 7.58-7.53 (m, 3H), 7.47 (t, J = 7.7 Hz, 1H), 7.34 (t, J = 7.2 Hz, 1H), 7.14 (d, |
| | | | | | J = 20.0 Hz, 1H), 6.92 (s, 1H), |
| | | | | | 4.65 (s, 4H), 4.61 (d, J = 8.6 Hz, |
| | | | | | 2H). |
| | | | | | LC/MS (ESI) m/z: 681 (M + H)⁺. |
| | | | | | RT (Method A): 1.99 min. |
| 298^{c,d} | 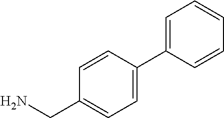 [SMILES: NCc1ccc(-c2ccccc2)cc1] | 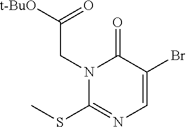 [SMILES: CSc1ncc(Br)c(=O)n1CC(=O)OC(C)(C)C] | 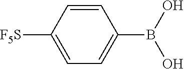 | 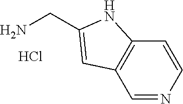 [SMILES: NCc1cc2cnccc2[nH]1 ; Cl] | ¹H NMR (400 MHz, DMSO-d₆) δ 11.36 (s, 1H), 8.76-8.72 (m, 2H), 8.15-8.12 (m, 1H), 7.88-7.85 (m, 2H), 7.68 (s, 1H), 7.66-7.62 (m, 5H), 7.49-7.47 (m, 2H), 7.46- 7.42 (m, 2H), 7.37-7.33 (m, 2H), 7.06 (s, 1H), 6.48 (t, J = 6.3 Hz, 1H), 6.36 (s, 1H), 4.52 (s, 2H), |
| | | | | | 4.45-4.40 (m, 4H). LC/MS (ESI) |
| | | | | | m/z: 667 (M + H)⁺. RT (Method |
| | | | | | A): 2.00 min. |
| 290^{a} | 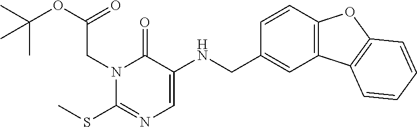 [SMILES: CSc1ncc(NCc2ccc3oc4ccccc4c3c2)c(=O)n1CC(=O)OC(C)(C)C] | | 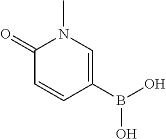 | 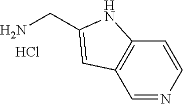 [SMILES: Cl ; NCc1cc2cnccc2[nH]1] | LC/MS (ESI) m/z: 600 (M + H)⁺. RT (Method A): 1.47 min. |
| 291^{a,h} | 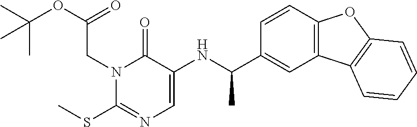 | | 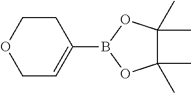 | 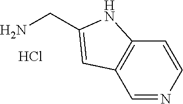 [SMILES: NCc1cc2cnccc2[nH]1 ; Cl] | LC/MS (ESI) m/z: 575 (M + H)⁺. RT (Method A): 1.54 min. |
| 309^{c,d} | 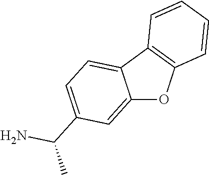 [SMILES: C[C@H](N)c1ccc2c(c1)oc1ccccc12] | 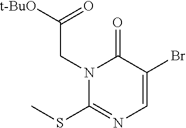 [SMILES: CSc1ncc(Br)c(=O)n1CC(=O)OC(C)(C)C] | 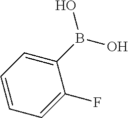 | 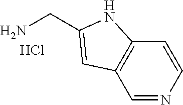 [SMILES: NCc1cc2cnccc2[nH]1 ; Cl] | ¹H NMR (400 MHz, DMSO-d₆) δ 11.32 (s, 1H), 8.74 (d, J = 11.4 Hz, 1H), 8.60 (s, 1H), 8.13 (s, 1H), 8.09 (d, J = 7.3 Hz, 2H), 7.79 (s, 1H), 7.68 (d, J = 7.9 Hz, 1H), 7.50 (dd, J = 11.4, 7.0 Hz, 3H), 7.38 (t, J = 7.5 Hz, 1H), 7.32 (d, J = 5.9 Hz, 2H), 7.27 (d, J = 8.0 Hz, 1H), 7.16-7.10 (m, 1H), 6.98 (s, 1H), 6.23 (d, J = 11.2 Hz, 1H), 6.13 (d, J = 7.9 Hz, 1H), 4.69 (dd, J = |
| | | | | | 11.7, 5.7 Hz, 2H), 4.33 (s, 3H), |
| | | | | | 1.60 (d, J = 6.1 Hz, 3H). LC/MS |
| | | | | | (ESI) m/z: 587 (M + H)⁺. RT |
| | | | | | (Method A): 1.73 min. |
| 325^{a,d} | 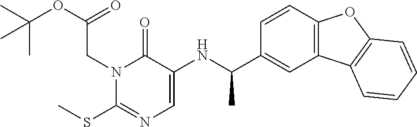 [SMILES: CSc1ncc(N[C@H](C)c2ccc3oc4ccccc4c3c2)c(=O)n1CC(=O)OC(C)(C)C] | | 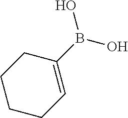 | 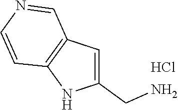 [SMILES: NCc1cc2cnccc2[nH]1 ; Cl] | ¹H NMR (400 MHz, DMSO-d₆) δ 11.31 (s, 1H), 8.74 (d, J = 9.9 Hz, 2H), 8.19 (s, 1H), 8.10 (d, J = 6.8 Hz, 2H), 7.70-7.63 (m, 2H), 7.59 (d, J = 8.2 Hz, 1H), 7.51 (t, J = 7.3 Hz, 1H), 7.39 (t, J = 7.3 Hz, 1H), 7.32 (s, 1H), 6.85 (s, 1H), 6.41 (s, 1H), 5.74 (d, J = 8.0 Hz, 2H), 4.71- |
| | | | | | 4.60 (m, 3H), 4.44 (s, 2H), 2.06 |
| | | | | | 2.03 (m, 2H), 1.95-1.92 (m, |
| | | | | | 2H), 1.58 (d, J = 5.7 Hz, 3H), 1.48- |
| | | | | | 1.46 (m, 4H). LC/MS (ESI) m/z: |
| | | | | | 573 (M + H)⁺. RT (Method A): |
| | | | | | 1.71 min. |
| 326^{a,d} | 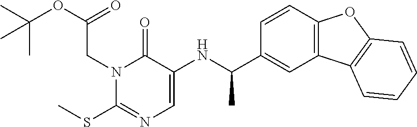 [SMILES: CSc1ncc(N[C@H](C)c2ccc3oc4ccccc4c3c2)c(=O)n1CC(=O)OC(C)(C)C] | | 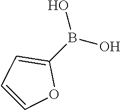 | 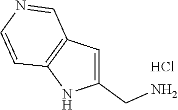 [SMILES: NCc1cc2cnccc2[nH]1 ; Cl] | ¹H NMR (400 MHz, CD₃OD) δ 8.71 (s, 1H), 8.10 (d, J = 5.3 Hz, 1H), 8.06 (s, 1H), 7.98 (d, J = 7.6 Hz, 1H), 7.57-7.50 (m, 3H), 7.49- 7.43 (m, 2H), 7.38 (d, J = 5.5 Hz, 1H), 7.36-7.31 (m, 1H), 6.92 (s, 1H), 6.77 (s, 1H), 6.53 (s, 1H), 6.43 (s, 1H), 4.95 (s, 2H), 4.71- |
| | | | | | 4.65 (m, 1H), 4.59 (s, 2H), 1.66 (d, |
| | | | | | J = 6.5 Hz, 3H). LC/MS (ESI) |
| | | | | | m/z: 559 (M + H)⁺. RT (Method |
| | | | | | A): 1.60 min. |
| 328^{a,d} | 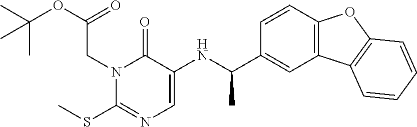 [SMILES: CSc1ncc(N[C@H](C)c2ccc3oc4ccccc4c3c2)c(=O)n1CC(=O)OC(C)(C)C] | | 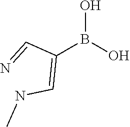 | 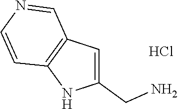 [SMILES: NCc1cc2cnccc2[nH]1 ; Cl] | ¹H NMR (400 MHz, DMSO-d₆) δ 11.37 (s, 1H), 8.83 (s, 1H), 8.75 (s, 1H), 8.20 (s, 1H), 8.10 (d, J = 8.4 Hz, 2H), 7.77 (s, 1H), 7.70- 7.63 (m, 2H), 7.60 (d, J = 8.5 Hz, 1H), 7.51 (s, 2H), 7.40 (d, J = 7.6 Hz, 1H), 7.33 (s, 1H), 6.94 (s, 1H), 6.40 (s, 1H), 5.78 (d, J = 6.7 Hz, |
| | | | | | 1H), 4.77 (d, J = 16.1 Hz, 1H), |
| | | | | | 4.66 (d, J = 17.1 Hz, 2H), 4.47 (s, |
| | | | | | 2H), 3.74 (s, 3H), 1.59 (d, J = 6.2 |
| | | | | | Hz, 3H). LC/MS (ESI) m/z: 573 |
| | | | | | (M + H)⁺. RT (Method A): 1.46 min. |
| 335^{a,d} | 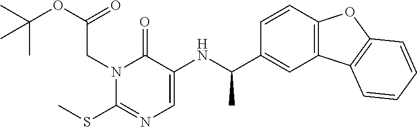 [SMILES: CSc1ncc(N[C@H](C)c2ccc3oc4ccccc4c3c2)c(=O)n1CC(=O)OC(C)(C)C] | | 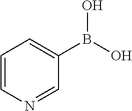 | 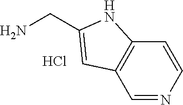 [SMILES: NCc1cc2cnccc2[nH]1 ; Cl] | ¹H NMR (400 MHz, DMSO-d₆) δ 11.36 (s, 1H), 8.75 (s, 2H), 8.60 (s, 2H), 8.23 (s, 1H), 8.10 (d, J = 7.7 Hz, 1H), 7.81 (d, J = 8.0 Hz, 1H), 7.71-7.60 (m, 3H), 7.52 (t, J = 7.7 Hz, 1H), 7.43-7.27 (m, 3H), 7.02 (s, 1H), 6.30 (s, 1H), 6.04 (d, J = 6.7 Hz, 1H), 4.68 (d, J = |
| | | | | | 6.1 Hz, 1H), 4.58 (m, 1H), 4.54- |
| | | | | | 4.37 (m, 3H), 1.61 (d, J = 6.2 |
| | | | | | Hz, 3H). LC/MS (ESI) m/z: 570 |
| | | | | | (M + H)⁺. RT (Method A): 1.47 |
| | | | | | min. |
| 342^{a,d} | 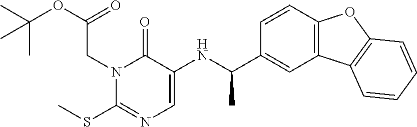 [SMILES: CSc1ncc(N[C@H](C)c2ccc3oc4ccccc4c3c2)c(=O)n1CC(=O)OC(C)(C)C] | | 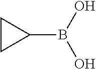 | 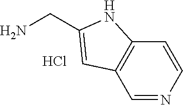 [SMILES: NCc1cc2cnccc2[nH]1 ; Cl] | ¹H NMR (400 MHz, DMSO-d₆) δ 1.37 (s, 1H), 8.84 (s, 1H), 8.74 (s, 1H), 8.17 (s, 1H), 8.10 (d, J = 7.2 Hz, 2H), 7.68 (d, J = 8.0 Hz, 1H), 7.63 (d, J = 8.1 Hz, 1H), 7.57 (d, J = 8.2 Hz, 1H), 7.51 (t, J = 7.8 Hz, 1H), 7.39 (t, J = 6.9 Hz, 1H), 7.33 (s, 1H), 6.76 (s, 1H), 6.44 (s, |
| | | | | | 1H), 5.58 (d, J = 6.5 Hz, 1H), 5.02- |
| | | | | | 4.84 (m, 2H), 4.59-4.51 (m, |
| | | | | | 1H), 4.48 (s, 2H), 1.85-1.75 (m, |
| | | | | | 1H), 1.56 (d, J = 5.6 Hz, 3H), 0.81- |
| | | | | | 0.68 (m, 4H). LC/MS (ESI) m/z: |
| | | | | | 533 (M + H)⁺. RT (Method A): |
| | | | | | 1.62 min. |
| 345^{a,d} | 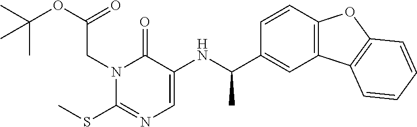 [SMILES: CSc1ncc(N[C@H](C)c2ccc3oc4ccccc4c3c2)c(=O)n1CC(=O)OC(C)(C)C] | | 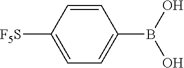 | 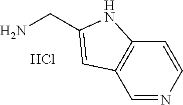 [SMILES: NCc1cc2cnccc2[nH]1 ; Cl] | ¹H NMR (400 MHz, CD₃OD) δ 8.71 (s, 1H), 8.12 (d, J = 5.6 Hz, 1H), 8.07 (s, 1H), 7.99 (d, J = 7.7 Hz, 1H), 7.71 (d, J = 8.1 Hz, 2H), 7.59-7.53 (m, 5H), 7.46 (t, J = 7.8 Hz, 1H), 7.40 (d, J = 5.8 Hz, 1H), 7.35 (d, J = 7.3 Hz, 1H), 6.94 (s, 1H), 6.51 (s, 1H), 4.69 (m, 1H), |
| | | | | | 4.60 (m, 2H), 4.55 (s, 2H), 1.68 (d, |
| | | | | | J = 6.5 Hz, 3H). LC/MS (ESI) |
| | | | | | m/z: 695 (M + H)⁺. RT (Method |
| | | | | | A): 2.05 min. |
| 378^{a,d,g} | 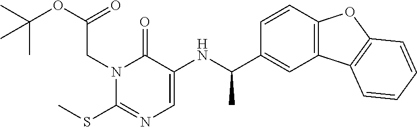 [SMILES: CSc1ncc(N[C@H](C)c2ccc3oc4ccccc4c3c2)c(=O)n1CC(=O)OC(C)(C)C] | | 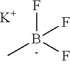 | 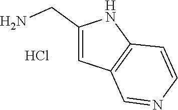 [SMILES: NCc1cc2cnccc2[nH]1 ; Cl] | ¹H NMR (400 MHz, DMSO-d₆) δ 12.11 (s, 1H), 9.00 (s, 1H), 8.94 (t, J = 5.5 Hz, 1H), 8.27 (d, J = 6.0 Hz, 1H), 8.16 (s, 1H), 8.08 (d, J = 7.6 Hz, 1H), 7.66 (m, 3H), 7.53 (m, 2H), 7.39 (t, J = 7.5 Hz, 1H), 6.77 (s, 1H), 6.68 (s, 1H), 5.52 (d, J = 7.0 Hz, 1H), 4.83-4.69 (m, |
| | | | | | 2H), 4.61-4.56 (m, 1H), 4.53 (m, |
| | | | | | 2H), 2.23 (s, 3H), 1.56 (d, J = 6.7 |
| | | | | | Hz, 3H). LC/MS (ESI) m/z: 507 |
| | | | | | (M + H)⁺. RT (Method A): 1.30 |
| | | | | | min. |

| | | | | |
|---|---|---|---|---|
| 404^{a,d,g} | 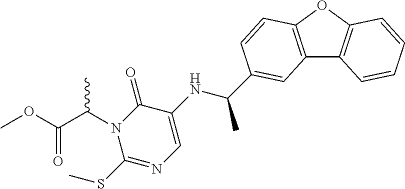 | 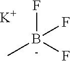 | 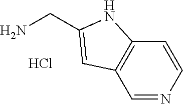 [SMILES: NCc1cc2cnccc2[nH]1 ; Cl] | ¹H NMR (400 MHz, CD₃OD) δ 8.79-8.69 (m, 1H), 8.12-8.07 (m, 1H), 8.06-7.74 (m, 2H), 7.57- 7.45 (m, 4H), 7.41-7.22 (m, 2H), 6.73-6.67 (m, 1H), 6.63- 6.53 (m, 1H), 5.13-4.91 (m, 1H), 4.72-4.40 (m, 3H), 2.41 (s, 3H), 1.71 (d, J = 6.8 Hz, 3H), 1.63 (dd, J = 14.2, 6.8 Hz, 3H). LC/MS (ESI) m/z: 521 (M + H)⁺. RT (Method A): 1.45 min. |

^{a}Steps 6-8 only.
^{b}Steps 4-8 only.
^{c}Steps 5-8 only.
^{d}The deprotection reaction was performed with LiOH..
^{e}The deprotection reaction was performed with TFA.
^{f}Step 6 was performed in THF.
^{g}Step 6 was performed in the presence of Pd(dppf)Cl₂, CuTC, and Cs₂CO₃ in DMF.
^{h}Reactant E was first deprotected to obtain the boronic acid.

The following compounds are prepared based on Steps 6-8 in Scheme 51:

| # | Reactant A | Reactant B | Reactant C |
|---|---|---|---|
| 121 | 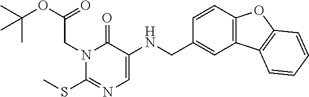 [SMILES: CSc1ncc(NCc2ccc3oc4ccccc4c3c2)c(=O)n1CC(=O)OC(C)(C)C] | 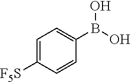 | 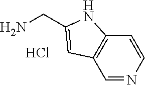 [SMILES: NCc1cc2cnccc2[nH]1 ; Cl] |
| 122 | 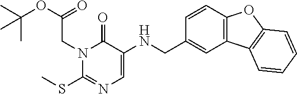 [SMILES: CSc1ncc(NCc2ccc3oc4ccccc4c3c2)c(=O)n1CC(=O)OC(C)(C)C] | 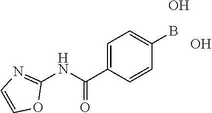 | 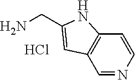 [SMILES: NCc1cc2cnccc2[nH]1 ; Cl] |
| 133 | 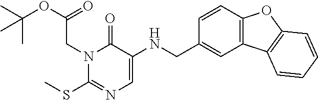 [SMILES: CSc1ncc(NCc2ccc3oc4ccccc4c3c2)c(=O)n1CC(=O)OC(C)(C)C] | 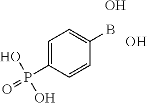 | 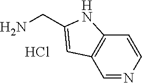 [SMILES: NCc1cc2cnccc2[nH]1 ; Cl] |
| 137 | 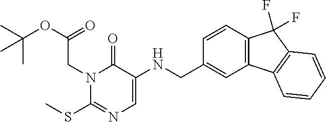 [SMILES: CSc1ncc(NCc2ccc3c(c2)-c2ccccc2C3(F)F)c(=O)n1CC(=O)OC(C)(C)C] | 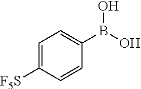 | 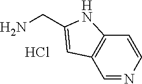 [SMILES: NCc1cc2cnccc2[nH]1 ; Cl] |
| 145 | 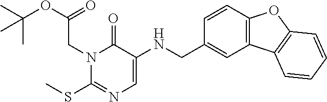 [SMILES: CSc1ncc(NCc2ccc3oc4ccccc4c3c2)c(=O)n1CC(=O)OC(C)(C)C] | 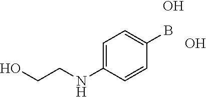 | 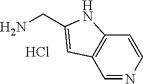 [SMILES: NCc1cc2cnccc2[nH]1 ; Cl] |
| 146 | 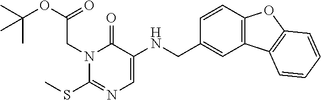 [SMILES: CSc1ncc(NCc2ccc3oc4ccccc4c3c2)c(=O)n1CC(=O)OC(C)(C)C] | 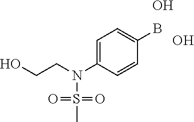 | 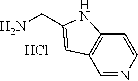 [SMILES: NCc1cc2cnccc2[nH]1 ; Cl] |
| 148 | 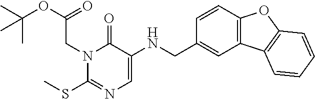 [SMILES: CSc1ncc(NCc2ccc3oc4ccccc4c3c2)c(=O)n1CC(=O)OC(C)(C)C] | 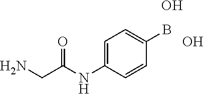 | 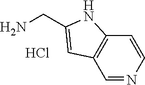 [SMILES: NCc1cc2cnccc2[nH]1 ; Cl] |
| 149 | 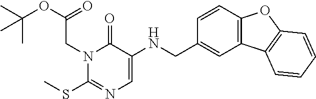 [SMILES: CSc1ncc(NCc2ccc3oc4ccccc4c3c2)c(=O)n1CC(=O)OC(C)(C)C] | 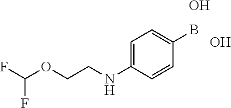 | 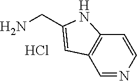 [SMILES: NCc1cc2cnccc2[nH]1 ; Cl] |
| 150 | 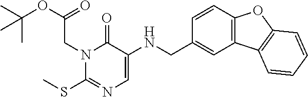 [SMILES: CSc1ncc(NCc2ccc3oc4ccccc4c3c2)c(=O)n1CC(=O)OC(C)(C)C] | 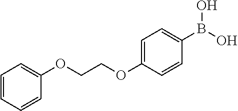 [SMILES: OB(O)c1ccc(OCCOc2ccccc2)cc1] | 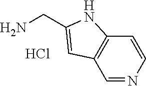 [SMILES: NCc1cc2cnccc2[nH]1 ; Cl] |
| 151 | 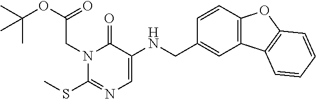 [SMILES: CSc1ncc(NCc2ccc3oc4ccccc4c3c2)c(=O)n1CC(=O)OC(C)(C)C] | 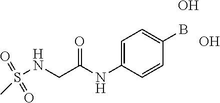 | 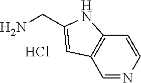 [SMILES: NCc1cc2cnccc2[nH]1 ; Cl] |
| 152 | 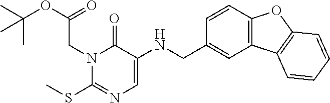 [SMILES: CSc1ncc(NCc2ccc3oc4ccccc4c3c2)c(=O)n1CC(=O)OC(C)(C)C] | 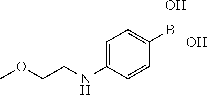 | 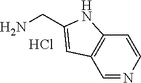 [SMILES: NCc1cc2cnccc2[nH]1 ; Cl] |
| 156 | 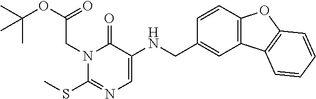 [SMILES: CSc1ncc(NCc2ccc3oc4ccccc4c3c2)c(=O)n1CC(=O)OC(C)(C)C] | 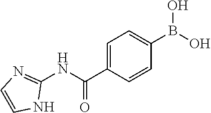 [SMILES: O=C(Nc1ncc[nH]1)c1ccc(B(O)O)cc1] | 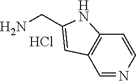 [SMILES: NCc1cc2cnccc2[nH]1 ; Cl] |
| 157 | 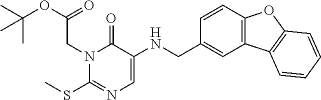 [SMILES: CSc1ncc(NCc2ccc3oc4ccccc4c3c2)c(=O)n1CC(=O)OC(C)(C)C] | 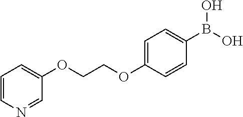 [SMILES: OB(O)c1ccc(OCCOc2cccnc2)cc1] | 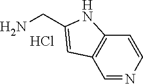 [SMILES: NCc1cc2cnccc2[nH]1 ; Cl] |
| 158 | 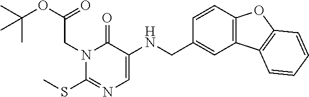 [SMILES: CSc1ncc(NCc2ccc3oc4ccccc4c3c2)c(=O)n1CC(=O)OC(C)(C)C] | 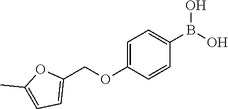 [SMILES: Cc1ccc(COc2ccc(B(O)O)cc2)o1] | 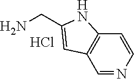 [SMILES: NCc1cc2cnccc2[nH]1 ; Cl] |
| 159 | 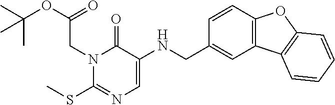 [SMILES: CSc1ncc(NCc2ccc3oc4ccccc4c3c2)c(=O)n1CC(=O)OC(C)(C)C] | 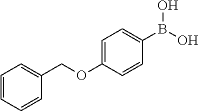 [SMILES: OB(O)c1ccc(OCc2ccccc2)cc1] | 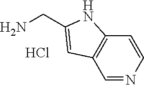 [SMILES: NCc1cc2cnccc2[nH]1 ; Cl] |
| 160 | 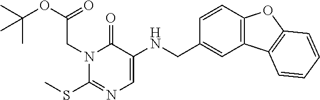 [SMILES: CSc1ncc(NCc2ccc3oc4ccccc4c3c2)c(=O)n1CC(=O)OC(C)(C)C] | 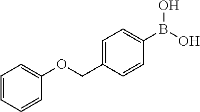 [SMILES: OB(O)c1ccc(COc2ccccc2)cc1] | 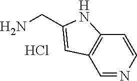 [SMILES: NCc1cc2cnccc2[nH]1 ; Cl] |
| 161 | 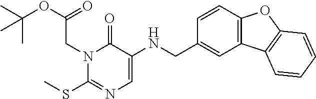 [SMILES: CSc1ncc(NCc2ccc3oc4ccccc4c3c2)c(=O)n1CC(=O)OC(C)(C)C] | 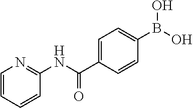 [SMILES: O=C(Nc1ccccn1)c1ccc(B(O)O)cc1] | 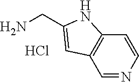 [SMILES: NCc1cc2cnccc2[nH]1 ; Cl] |
| 162 | 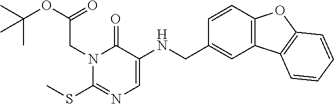 [SMILES: CSc1ncc(NCc2ccc3oc4ccccc4c3c2)c(=O)n1CC(=O)OC(C)(C)C] | 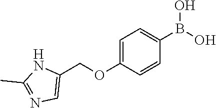 [SMILES: Cc1ncc(COc2ccc(B(O)O)cc2)[nH]1] | 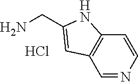 [SMILES: NCc1cc2cnccc2[nH]1 ; Cl] |

### Scheme 52. Synthesis of 2-(5-((dibenzo[b,d]furan-2-ylmethyl)amino)-6-oxo-2-phenylpyrimidin-1(6H)-yl)-N-(pyrrolo[1,2-a]pyrazin-7-ylmethyl)acetamide (Compound 144)

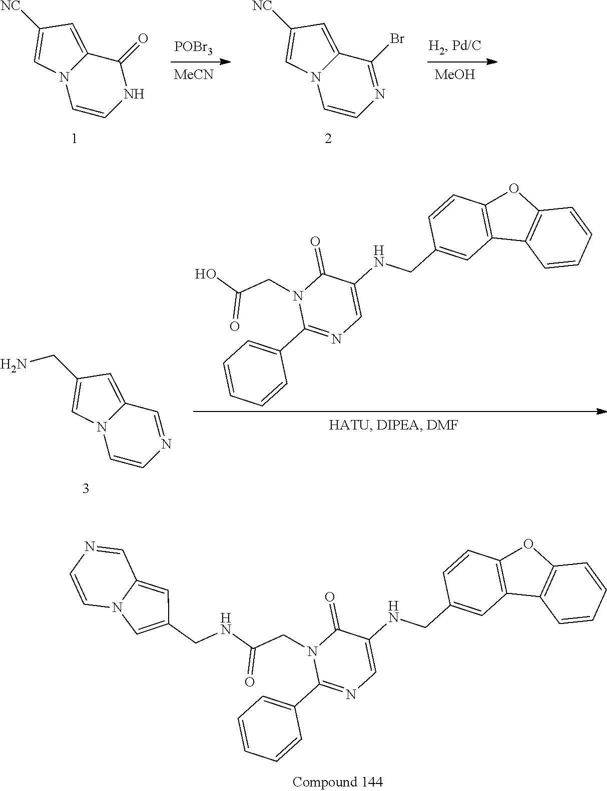

### Step 1: 1-Bromopyrrolo[1,2-a]pyrazine-7-carbonitrile (2)

To a solution of 1-oxo-1,2-dihydropyrrolo[1,2-a]pyrazine-7-carbonitrile (1.0 g, 6.29 mmol) in MeCN (10 mL) was added POBr₃ (1.9 g, 12.57 mmol) under N₂ atmosphere and the mixture was stirred at 90° C. overnight. The reaction mixture was filtered, and the filter cake was washed with MeOH, dried under vacuum to give the title compound (1.0 g, yield 71.4%) as a yellow solid. LC/MS (ESI) m/z: 222 (M+H)⁺.

### Step 2: Pyrrolo[1,2-a]pyrazin-7-ylmethanamine (3)

To a solution of 1-bromopyrrolo[1,2-a]pyrazine-7-carbonitrile (100 mg, 0.45 mmol) in MeOH (4 mL) was added Pd/C (20 mg, 10% wt.), the mixture was degassed under N₂ atmosphere for three times and stirred under a H₂ balloon at room temperature overnight. The mixture was filtered, and the filtrate was concentrated under reduced pressure to dryness to give the title compound (50 mg, yield 75.1%) as a colorless oil, which was used directly in the next step without further purification. LC/MS (ESI) m/z: 148 (M+H)⁺.

### Step 3: 2-(5-((Dibenzo[b,d]furan-2-ylmethyl)amino)-6-oxo-2-phenylpyrimidin-1(6H)-yl)-N-(pyrrolo[1,2-a]pyrazin-7-ylmethyl)acetamide (Compound 144)

To a mixture of 2-(5-((dibenzo[b,d]furan-2-ylmethyl)amino)-6-oxo-2-phenylpyrimidin-1(6H)-yl)acetic acid (72 mg, 0.17 mmol) and pyrrolo[1,2-a]pyrazin-7-ylmethanamine (50 mg, 0.34 mmol) in DMF (2 mL) was added DIPEA (109 mg, 0.85 mmol) and HATU (84 mg, 0.22 mmol) and the mixture was stirred at room temperature for 1 hour. The mixture was concentrated under reduced pressure to dryness. The residue was purified by prep-TLC (DCM:MeOH=8:1) and further purified by prep-HPLC to give Compound 144 (1.2 mg, yield 1.3%) as a white solid. ¹H NMR (400 MHz, CD₃OD) δ 8.08 (s, 1H), 8.03 (d, J=7.6 Hz, 1H), 7.56 (t, J=7.4 Hz, 3H), 7.49 (dd, J=16.7, 9.2 Hz, 2H), 7.38 (dd, J=13.9, 7.0 Hz, 7H), 7.34 (s, 2H), 7.10 (s, 1H), 6.22 (d, J=36.7 Hz, 1H), 4.58 (s, 2H), 4.01 (t, J=4.9 Hz, 1H), 3.92 (t, J=4.5 Hz, 2H), 3.80 (d, J=5.9 Hz, 1H). LC/MS (ESI) (m/z): 555 (M+H)⁺. RT (Method A): 2.48 min.

### Scheme 53. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-2-(3-fluorophenyl)oxazole-4-carboxamide (Compound 205)

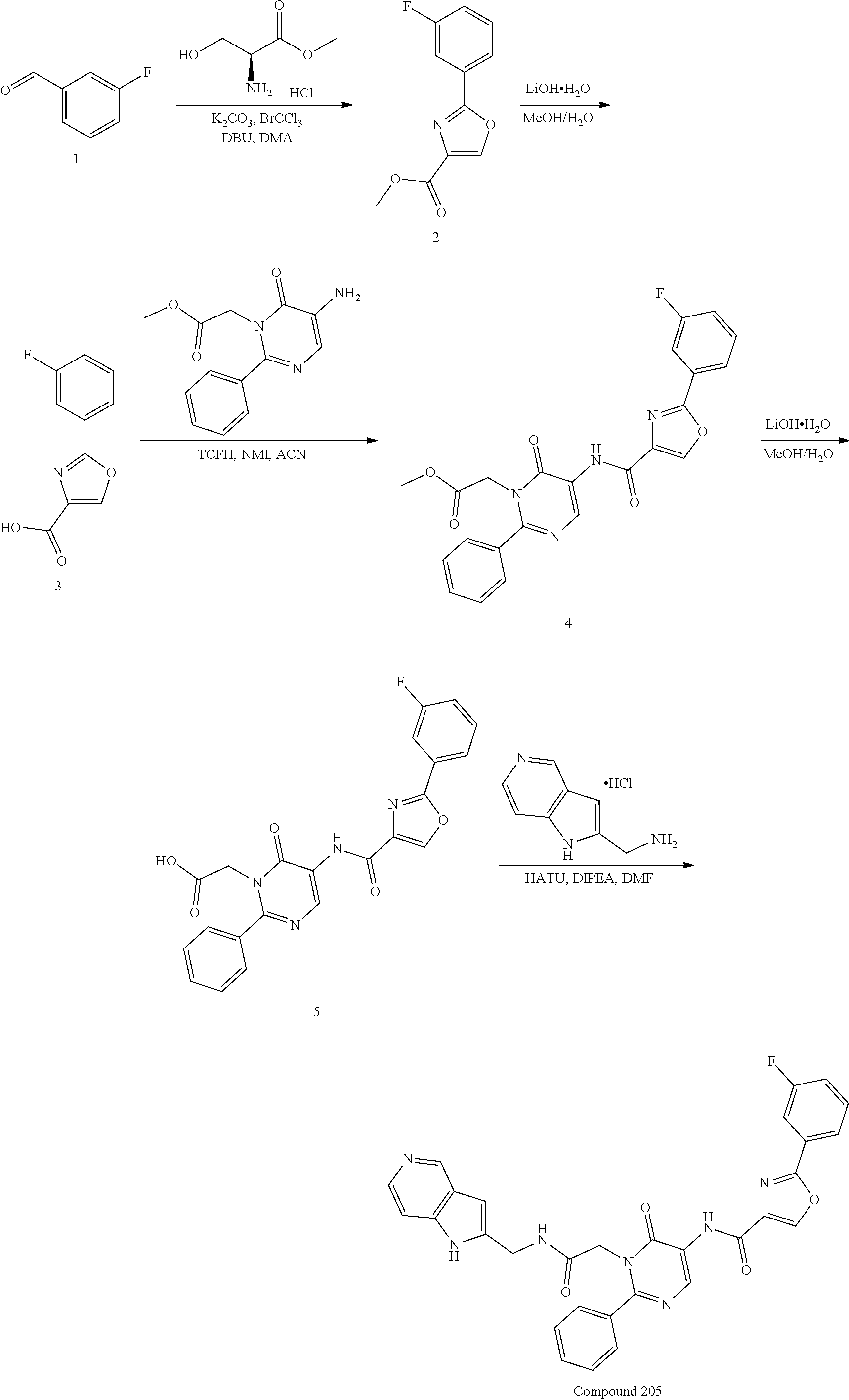

### Step 1: Methyl 2-(3-fluorophenyl)oxazole-4-carboxylate (2)

To a mixture of 3-fluorobenzaldehyde (1.24 g, 10.0 mmol) and methyl L-serinate hydrochloride (1.56 g, 10.0 mmol) in DMA (20 mL) was added K₂CO₃ (2.76 g, 20.0 mmol) under N₂ atmosphere and the mixture was stirred at room temperature overnight. Afterwards, to the reaction mixture was added BrCCl₃ (2.90 mL, 30.0 mmol) and DBU (4.60 mL, 30.0 mmol) at 0° C. and the reaction mixture was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give the title compound (760 mg, yield 34.2%) as a yellow solid. LC/MS (ESI) m/z: 222 (M+H)⁺.

### Step 2: 2-(3-Fluorophenyl)oxazole-4-carboxylic acid (3)

To a solution of methyl 2-(3-fluorophenyl)oxazole-4-carboxylate (100 mg, 0.45 mmol) in MeOH/H₂O (1 mL, v/v=4/1) was added LiOH·H₂O (38 mg, 0.90 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (90 mg, yield 96.2%) as a yellow solid, which was used directly in the next step without further purification. LC/MS (ESI) m/z: 208 (M+H)⁺.

### Step 3: Methyl 2-(5-(2-(3-fluorophenyl)oxazole-4-carboxamido)-6-oxo-2-phenylpyrimidin-1(6H)-yl)acetate (4)

To a mixture of 2-(3-fluorophenyl)oxazole-4-carboxylic acid (50 mg, 0.24 mmol) and methyl 2-(5-amino-6-oxo-2-phenylpyrimidin-1(6H)-yl)acetate (63 mg, 0.24 mmol) in CH₃CN (1 mL) was added NMI (60 mg, 0.73 mmol) and TCFH (203 mg, 0.73 mmol) and the mixture was stirred at room temperature for 1 hour. The mixture was diluted with DCM, washed with saturated aq.NaHCO₃ solution and brine, dried over anhydrous Na₂SO₄, filtered, and concentrated under reduced pressure to dryness. The residue was purified by flash chromatography (silica gel, 0-7% MeOH in DCM) to give the title compound (50 mg, yield 46.4%) as a yellow solid. LC/MS (ESI) m/z: 449 (M+H)⁺.

### Step 4: 2-(5-(2-(3-Fluorophenyl)oxazole-4-carboxamido)-6-oxo-2-phenylpyrimidin-1(6H)-yl)acetic acid (5)

To a solution of methyl 2-(5-(2-(3-fluorophenyl)oxazole-4-carboxamido)-6-oxo-2-phenylpyrimidin-1(6H)-yl)acetate (50 mg, 0.11 mmol) in MeOH/water (1 mL, v/v=4/1) was added LiOH·H₂O (9 mg, 0.22 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (46 mg, yield 96.1%) as a yellow solid, which was used directly in the next step without further purification. LC/MS (ESI) m/z: 435 (M+H)⁺.

### Step 5: N-(1-(2-(((1H-pyrrolo[3,2-c]pyridine-2-yl)methyl)amino)-2-oxoethyl)-6-oxo-2-phenyl-1,6-dihydropyrimidin-5-yl)-2-(3-fluorophenyl)oxazole-4-carboxamide (Compound 205)

To a mixture of 2-(5-(2-(3-fluorophenyl)oxazole-4-carboxamido)-6-oxo-2-phenylpyrimidin-1(6H)-yl)acetic acid (30 mg, 0.069 mmol) and (1H-pyrrolo[3,2-c]pyridine-2-yl)methanamine hydrochloride (15 mg, 0.10 mmol) in DMF (1 mL) was added DIPEA (45 mg, 0.35 mmol) and HATU (40 mg, 0.10 mmol) under N₂ atmosphere and the reaction mixture was stirred at room temperature for 2 hours. The mixture was concentrated under reduced pressure to dryness. The residue was purified by prep-HPLC to give Compound 205 (6.6 mg, yield 17.0%) as a white solid. ¹H NMR (400 MHz, DMSO-d₆) δ 11.35 (s, 1H), 9.47 (s, 1H), 9.07 (s, 1H), 8.97 (s, 1H), 8.80 (t, J=5.5 Hz, 1H), 8.76 (s, 1H), 8.11 (d, J=5.6 Hz, 1H), 7.94 (d, J=7.9 Hz, 1H), 7.84 (d, J=8.8 Hz, 1H), 7.68 (dd, J=10.9, 5.1 Hz, 1H), 7.58-7.55 (m, 3H), 7.53 (dd, J=6.0, 1.9 Hz, 1H), 7.48 (d, J=7.1 Hz, 2H), 7.33 (d, J=5.6 Hz, 1H), 6.35 (s, 1H), 4.61 (s, 2H), 4.45 (d, J=5.4 Hz, 2H). LC/MS (ESI) m/z: 564 (M+H)⁺. RT (Method A): 0.94 min.

The following compounds were prepared based on Steps 3-5 in Scheme 53:

| # | Reactant A | Reactant B | Reactant C | Characterization Data |
|---|---|---|---|---|
| 97 | 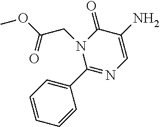 | 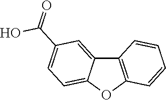 [SMILES: O=C(O)c1ccc2oc3ccccc3c2c1] | 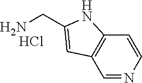 [SMILES: NCc1cc2cnccc2[nH]1 ; Cl] | ¹H NMR (400 MHz, DMSO-d₆) δ 11.35 (s, 1H), 9.66 (s, 1H), 8.90-8.85 (m, 2H), 8.81-8.73 (m, 2H), 8.30 (d, J = 7.4 Hz, 1H), 8.17 (dd, J = 8.7, 1.8 Hz, 1H), 8.11 (d, J = 5.6 Hz, 1H), 7.87 (d, J = 8.6 Hz, 1H), 7.79 (d, J = 8.3 Hz, 1H), 7.63-7.54 (m, 4H), 7.51-7.46 (m, 3H), 7.33 (d, J = 5.6 Hz, 1H), 6.34 (s, 1H), 4.61 (s, 2H), 4.45 (d, J = 5.4 Hz, 2H). LC/MS (ESI) m/z: 569 (M + H)⁺. RT (Method A): 0.26 min. |
| 165 | 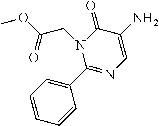 | 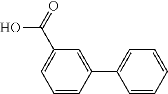 [SMILES: O=C(O)c1cccc(-c2ccccc2)c1] | 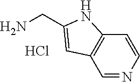 [SMILES: NCc1cc2cnccc2[nH]1 ; Cl] | ¹H NMR (400 MHz, CD₃OD) δ 9.07 (s, 1H), 8.71 (s, 1H), 8.22 (s, 1H), 8.08 (d, J = 5.9 Hz, 1H), 7.95 (d, J = 8.0 Hz, 1H), 7.90 (d, J = 8.1 Hz, 1H), 7.72 (d, J = 7.5 Hz, 2H), 7.64 (t, J = 7.8 Hz, 1H), 7.57 (d, J = 7.2 Hz, 2H), 7.53-7.49 (m, 2H), 7.46 (d, J = 9.2 Hz, 2H), 7.44-7.36 (m, 3H), 6.50 (s, 1H), 4.72 (s, 2H), 4.56 (s, 2H). LC/MS (ESI) m/z: 555 (M + H)⁺. |
| 169 | 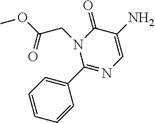 | 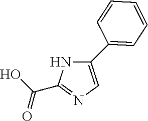 [SMILES: O=C(O)c1ncc(-c2ccccc2)[nH]1] | 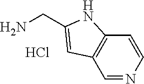 [SMILES: NCc1cc2cnccc2[nH]1 ; Cl] | ¹H NMR (400 MHz, DMSO-d₆) δ 11.36 (s, 1H), 9.70 (s, 1H), 8.99 (s, 1H), 8.82 (s, 1H), 8.77 (s, 1H), 8.19 (s, 1H), 8.12 (d, J = 5.0 Hz, 1H), 8.01 (s, 1H), 7.91 (d, J = 7.4 Hz, 2H), 7.56 (s, 3H), 7.49 (d, J = 7.2 Hz, 2H), 7.45 (s, 2H), 7.35 (d, J = 5.7 Hz, 2H), 6.37 (s, 1H), 4.62 (s, 2H), 4.46 (d, J = 5.4 Hz, 2H). LC/MS (ESI) m/z: 545 (M + H)⁺. RT (Method A): 1.17 min. |
| 170^{a} | 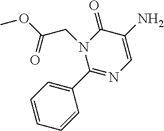 | 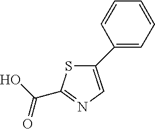 [SMILES: O=C(O)c1ncc(-c2ccccc2)s1] | 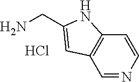 [SMILES: NCc1cc2cnccc2[nH]1 ; Cl] | ¹H NMR (400 MHz, DMSO-d₆) δ 11.37 (s, 1H), 9.73 (s, 1H), 8.93 (s, 1H), 8.79 (d, J = 5.4 Hz, 1H), 8.76 (s, 1H), 8.59 (s, 1H), 8.16-8.12 (m, 2H), 7.85 (d, J = 7.0 Hz, 2H), 7.57-7.53 (m, 4H), 7.48 (d, J = 7.1 Hz, 3H), 7.34 (d, J = 5.7 Hz, 1H), 6.33 (s, 1H), 4.62 (s, 2H), 4.44 (d, J = 5.6 Hz, 2H). LC/MS (ESI) m/z: 562 (M + H)⁺. RT (Method A): 1.61 min. |
| 175 | 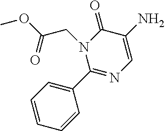 | 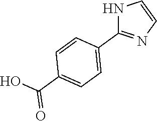 [SMILES: O=C(O)c1ccc(-c2ncc[nH]2)cc1] | 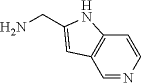 [SMILES: NCc1cc2cnccc2[nH]1] | ¹H NMR (400 MHz, DMSO-d₆) δ 12.75 (s, 1H), 11.35 (s, 1H), 9.55 (s, 1H), 8.85-8.73 (m, 3H), 8.13-8.04 (m, 5H), 7.61-7.52 (m, 3H), 7.47 (t, J = 7.4 Hz, 2H), 7.35-7.00 (m, 3H), 6.33 (s, 1H), 4.60 (s, 2H), 4.43 (d, J = 5.4 Hz, 2H). LC/MS (ESI) m/z: 545 (M + H)⁺. |
| 209 | 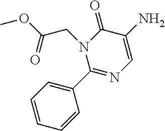 | 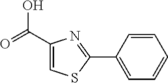 [SMILES: O=C(O)c1csc(-c2ccccc2)n1] | 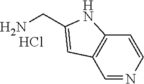 [SMILES: NCc1cc2cnccc2[nH]1 ; Cl] | ¹H NMR (400 MHz, DMSO-d₆) δ 11.35 (s, 1H), 10.03 (s, 1H), 8.82 (s, 1H), 8.80-8.74 (m, 2H), 8.69 (s, 1H), 8.12 (d, J = 5.6 Hz, 1H), 8.07-8.01 (m, 2H), 7.60-7.53 (m, 6H), 7.52-7.44 (m, 2H), 7.33 (d, J = 5.6 Hz, 1H), 6.34 (s, 1H), 4.59 (s, 2H), 4.44 (d, J = 5.5 Hz, 2H). LC/MS (ESI) m/z: 562 (M + H)⁺. RT (Method A): 1.38 min. |
| 213 | 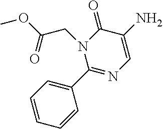 [SMILES: COC(=O)Cn1c(-c2ccccc2)ncc(N)c1=O] | 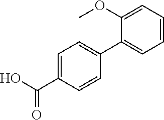 [SMILES: COc1ccccc1-c1ccc(C(=O)O)cc1] | 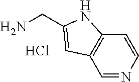 [SMILES: NCc1cc2cnccc2[nH]1 ; Cl] | ¹H NMR (400 MHz, DMSO-d₆) δ 11.33 (s, 1H), 9.52 (s, 1H), 8.85 (s, 1H), 8.79-8.74 (m, 2H), 8.12 (d, J = 5.6 Hz, 1H), 8.01 (d, J = 8.2 Hz, 2H), 7.66 (d, J = 8.2 Hz, 2H), 7.58 (d, J = 7.4 Hz, 2H), 7.54 (d, J = 7.2 Hz, 1H), 7.47 (m, 2H), 7.41 (m, 1H), 7.37 (d, J = 7.6 Hz, 1H), 7.32 (d, J = 5.6 Hz, 1H), 7.16 (d, J = 8.3 Hz, 1H), 7.08 (m, 1H), 6.33 (s, 1H), 4.60 (s, 2H), 4.44 (d, J = 5.5 Hz, 2H), |
| | | | | 3.80 (s, 3H). LC/MS (ESI) m/z: 585 (M + H)⁺. |
| 214 | 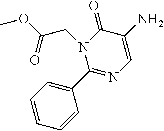 | 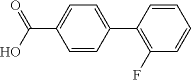 [SMILES: O=C(O)c1ccc(-c2ccccc2F)cc1] | 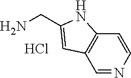 [SMILES: NCc1cc2cnccc2[nH]1 ; Cl] | ¹H NMR (400 MHz, DMSO-d₆) δ 11.34 (s, 1H), 9.62 (s, 1H), 8.84 (s, 1H), 8.80-8.76 (m, 1H), 8.76-8.74 (m, 1H), 8.13-8.11 (m, 1H), 8.11- 8.08 (m, 2H), 7.76-7.75 (m, 1H), 7.74-7.73 (m, 1H), 7.66-7.61 (m, 1H), 7.60-7.58 (m, 1H), 7.58-7.57 (m, 1H), 7.56-7.52 (m, 1H), 7.51-7.46 (m, 3H), 7.40-7.35 (m, 2H), 7.33- 7.31 (m, 1H), 6.33 (s, 1H), 4.60 (s, 2H), 4.44 (d, J = 5.6 Hz, 2H). LC/MS (ESI) m/z: 573 (M + H)⁺. |
| 243^{a} | 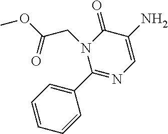 | 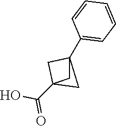 [SMILES: O=C(O)C12CC(c3ccccc3)(C1)C2] | 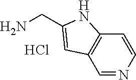 [SMILES: NCc1cc2cnccc2[nH]1 ; Cl] | ¹H NMR (400 MHz, CD₃OD) δ 8.90 (s, 1H), 8.75 (d, J = 0.8 Hz, 1H), 8.14 (d, J = 5.9 Hz, 1H), 7.57-7.51 (m, 3H), 7.45 (t, J = 7.1 Hz, 3H), 7.35- 7.24 (m, 5H), 6.52 (s, 1H), 4.70 (s, 2H), 4.57 (s, 2H), 2.43 (s, 6H). LC/MS (ESI) m/z: 545 (M + H)⁺. RT (Method A): 1.39 min. |
| 254 | 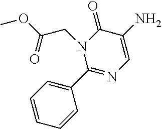 | 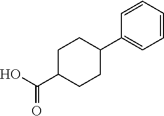 [SMILES: O=C(O)C1CCC(c2ccccc2)CC1] | 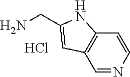 [SMILES: NCc1cc2cnccc2[nH]1 ; Cl] | ¹H NMR (400 MHz, CD₃OD) δ 8.95 (d, J = 4.1 Hz, 1H), 8.82 (d, J = 8.0 Hz, 1H), 8.18 (d, J = 5.9 Hz, 1H), 7.56-7.50 (m, 4H), 7.45 (d, J = 7.3 Hz, 2H), 7.25 (m, 4H), 7.18-7.12 (m, 1H), 6.62 (s, 1H), 4.69 (s, 2H), 4.58 (s, 2H), 2.69-2.52 (m, 2H), 2.17-2.04 (m, 2H), 1.98 (d, J = 12.8 Hz, 2H), 1.80-1.70 (m, 2H), 1.69-1.57 (m, 2H). LC/MS (ESI) m/z: 561 (M + H)⁺. |
| | | | | RT (Method A): 1.57 min. |
| 392^{a} | 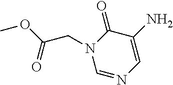 | 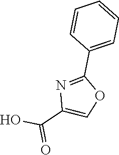 [SMILES: O=C(O)c1coc(-c2ccccc2)n1] | 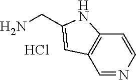 [SMILES: NCc1cc2cnccc2[nH]1 ; Cl] | ¹H NMR (400 MHz, DMSO-d₆) δ 11.43 (s, 1H), 9.44 (s, 1H), 9.00 (s, 2H), 8.88 (s, 1H), 8.75 (s, 1H), 8.31 (d, J = 9.4 Hz, 1H), 8.11 (d, J = 5.6 Hz, 1H), 8.09-8.05 (m, 2H), 7.63-7.59 (m, 3H), 7.33 (d, J = 5.5 Hz, 1H), 6.47 (s, 1H), 4.78 (s, 2H), 4.50 (d, J = 5.4 Hz, 2H). LC/MS (ESI) m/z: 470 (M + H)⁺. RT (Method A): 1.00 min. |
| 393^{a} | 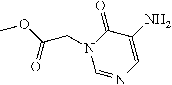 | 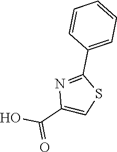 [SMILES: O=C(O)c1csc(-c2ccccc2)n1] | 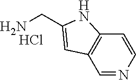 [SMILES: NCc1cc2cnccc2[nH]1 ; Cl] | ¹H NMR (400 MHz, DMSO-d₆) δ 11.39 (s, 1H), 9.89 (s, 1H), 8.95 (t, J = 5.5 Hz, 1H), 8.92 (s, 1H), 8.74 (s, 1H), 8.59 (s, 1H), 8.30 (s, 1H), 8.10 (d, J = 5.6 Hz, 1H), 8.05-8.02 (m, 2H), 7.60- 7.57 (m, 3H), 7.32 (d, J = 5.5 Hz, 1H), 6.48 (s, 1H), 4.78 (s, 2H), 4.50 (d, J = 5.5 Hz, 2H). LC/MS (ESI) m/z: 486 (M + H)⁺. RT (Method A): 1.11 min. |
| 405^{a} | 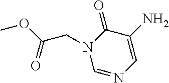 | 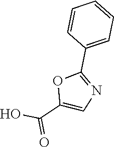 [SMILES: O=C(O)c1cnc(-c2ccccc2)o1] | 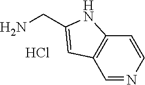 [SMILES: NCc1cc2cnccc2[nH]1 ; Cl] | ¹H NMR (400 MHz, DMSO-d₆) δ 11.45 (s, 1H), 9.00 (t, J = 5.4 Hz, 1H), 8.74 (d, J = 0.9 Hz, 1H), 8.64 (s, 1H), 8.32 (s, 1H), 8.15-8.09 (m, 4H), 7.63-7.59 (m, 3H), 7.32 (d, J = 5.6 Hz, 1H), 6.46 (s, 1H), 4.76 (s, 2H), 4.49 (d, J = 5.3 Hz, 2H). LC/MS (ESI) m/z: 470 (M + H)⁺. RT (Method A): 0.77 min. |

^{a}Step 4 was performed with TBD in a mixture of MeCN and H₂O.

### Scheme 54. Synthesis of N-((1H-pyrrolo[3,2-c]pyridine-2-yl)methyl)-2-(2-phenyl-5-((3-phenylpropyl)amino)146yridine-3-yl)acetamide (Compound 207)

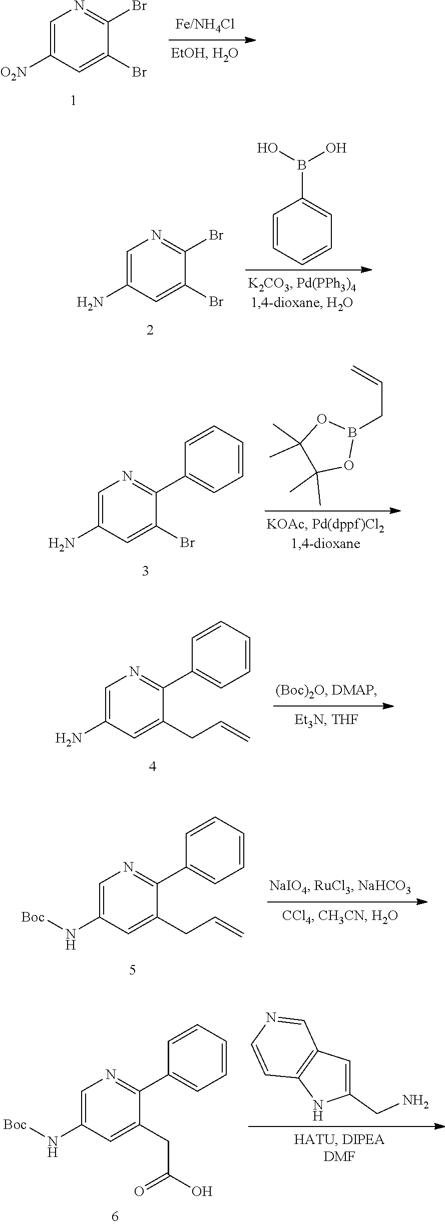

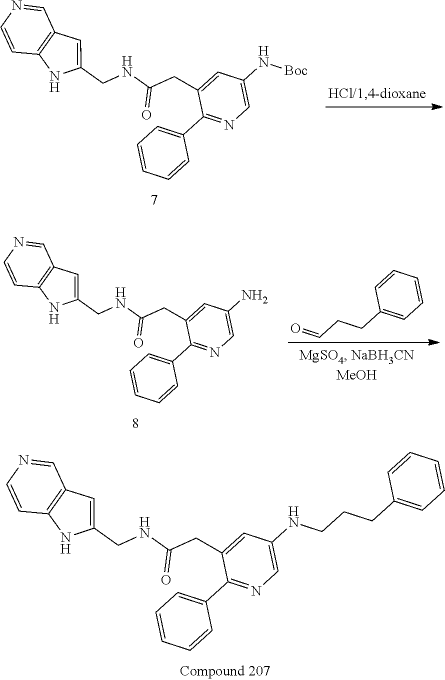 [SMILES: CC(C)(C)OC(=O)Nc1cnc(-c2ccccc2)c(CC(=O)NCc2cc3cnccc3[nH]2)c1 ; Nc1cnc(-c2ccccc2)c(CC(=O)NCc2cc3cnccc3[nH]2)c1 ; O=CCCc1ccccc1 ; O=C(Cc1cc(NCCCc2ccccc2)cnc1-c1ccccc1)NCc1cc2cnccc2[nH]1]

### Step 1: 5,6-Dibromopyridin-3-amine (2)

To a mixture of 2,3-dibromo-5-nitropyridine (15 g, 53.21 mmol) and Fe (8.94 g, 159.6 mmol) in EtOH (75 mL) and water (7.5 mL) was added NH₄Cl (5.7 g, 106.42 mmol) and the mixture was stirred at 85° C. overnight. The mixture was filtered, and the filter cake was washed with EtOAc twice. The filtrate was washed with water and brine, dried over anhydrous Na₂SO₄, filtered, and concentrated under reduced pressure to dryness. The residue was purified by flash chromatography (silica gel, 0-30% EtOAc in PE) to give the title compound (9.40 g, yield 70.1%) as a yellow solid. LC/MS (ESI) m/z: 251 (M+H)⁺.

### Step 2: 5-Bromo-6-phenylpyridin-3-amine (3)

To a mixture of 5,6-dibromopyridin-3-amine (9.40 g, 37.6 mmol) and phenylboronic acid (5.07 g, 41.5 mmol) in 1,4-dioxane (100 mL) and water (50 mL) was added K₂CO₃ (15.63 g, 113.25 mmol) and Pd(PPh₃)₄ (4.36 g, 3.77 mmol) under N₂ atmosphere, the reaction mixture was degassed under N₂ atmosphere for three times and stirred at 80° C. for 2 hours. The mixture was diluted EtOAc, washed with water and brine, dried over anhydrous Na₂SO₄, filtered, and concentrated under reduced pressure to dryness. The residue was purified by flash chromatography (silica gel, 0-70% EtOAc in PE) to give the title compound (5.6 g, yield 59.6%) as a white solid. LC/MS (ESI) m/z: 249 (M+H)⁺.

### Step 3: 5-Allyl-6-phenylpyridin-3-amine (4)

To a mixture of 5-bromo-6-phenylpyridin-3-amine (5.60 g, 22.58 mmol) and 2-allyl-4,4,5,5-tetramethyl-1,3,2-dioxaborolane (7.59 g, 45.16 mmol) in 1,4-dioxane (50 mL) was added KOAc (9.35 mg, 67.74 mmol) and Pd(dppf)Cl₂ (3.30 mg, 4.52 mmol) under N₂ atmosphere, the reaction mixture was degassed under N₂ atmosphere for three times and stirred at 90° C. for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (2.30 g, yield 55.5%) as a colorless oil. LC/MS (ESI) m/z: 211 (M+H)⁺.

### Step 4: Tert-butyl (5-allyl-6-phenylpyridin-3-yl)carbamate (5)

To a mixture of 5-allyl-6-phenylpyridin-3-amine (2.3 g, 10.95 mmol) and (Boc)₂O (7.16 g, 32.86 mmol) in THF (30 mL) was added DMAP (134 mg, 1.10 mmol) and the mixture was stirred at room temperature for 6 hours. The mixture was concentrated under reduced pressure to dryness. The residue was purified by flash chromatography (silica gel, 0-30% EtOAc in PE) to give the title compound (960 mg, yield 28.2%) as a white solid. LC/MS (ESI) m/z: 311 (M+H)⁺.

### Step 5: 2-(5-((Tert-butoxycarbonyl)amino)-2-phenylpyridin-3-yl)acetic acid (6)

To a solution of tert-butyl (5-allyl-6-phenylpyridin-3-yl)carbamate (950 mg, 2.32 mmol) in CCl₄ (10 mL) and MeCN (10 mL) was added NaIO₄ (2.48 g, 11.56 mmol) followed by the addition of a solution of RuCl₃ (25 mg, 0.12 mmol) in water (5 mL) at 25° C. and the reaction mixture was stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (150 mg, yield 19.7%) as a colorless oil. LC/MS (ESI) m/z: 329 (M+H)⁺.

### Step 6: Tert-butyl (5-(2-(((1H-pyrrolo[3,2-c]pyridine-2-yl)methyl)amino)-2-oxoethyl)-6-phenylpyridin-3-yl)carbamate (7)

To a solution of 2-(5-((tert-butoxycarbonyl)amino)-2-phenylpyridin-3-yl)acetic acid (80 mg, 0.24 mmol) in DMF (2 mL) was added (1H-pyrrolo[3,2-c]pyridine-2-yl)methanamine (45 mg, 0.24 mmol), HATU (139 mg, 0.36 mmol) and DIPEA (189 mg, 1.46 mmol) and the mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give the title compound (20 mg, yield 17.8%) as a white solid. LC/MS (ESI) m/z: 458 (M+H)⁺.

### Step 8: N-((1H-pyrrolo[3,2-c]pyridine-2-yl)methyl)-2-(5-amino-2-phenylpyridin-3-yl)acetamide (8)

To a solution of tert-butyl (5-(2-(((1H-pyrrolo[3,2-c]pyridine-2-yl)methyl)amino)-2-oxoethyl)-6-phenylpyridin-3-yl)carbamate (20 mg, 0.04 mmol) in MeOH (2 mL) was added HCl/1,4-dioxane (1.0 mL, 4M) at 25° C. under N₂ atmosphere and the reaction mixture was stirred at room temperature for 3 hours. The reaction mixture was concentrated under reduced pressure to dryness to give the title compound (15 mg, yield 96.2%) as a yellow solid, which was used directly in the next step without further purification. LC/MS (ESI) m/z: 358 (M+H)⁺.

### Step 9: N-((1H-pyrrolo[3,2-c]pyridine-2-yl)methyl)-2-(2-phenyl-5-((3-phenylpropyl) amino) 148 yridine-3-yl)acetamide (Compound 207)

To a solution of N-((1H-pyrrolo[3,2-c]pyridine-2-yl)methyl)-2-(5-amino-2-phenylpyridin-3-yl)acetamide (15 mg, 0.04 mmol) in MeOH (2 mL) was added 3-phenylpropanal (21 mg, 0.12 mmol), NaBH₃CN (19 mg, 0.12 mmol) and MgSO₄ (50 mg) successively at 25° C. under N₂ atmosphere and the reaction mixture was stirred at 50° C. for 3 hours. The mixture was filtered, and the filtrate was concentrated under reduced pressure to dryness. The residue was purified by prep-HPLC to give Compound 207 (2.0 mg, yield 10.0%) as a white solid. ¹H NMR (400 MHz, CD₃OD) δ 8.69 (s, 1H), 8.10 (d, J=5.8 Hz, 1H), 7.86 (d, J=2.6 Hz, 1H), 7.37-7.34 (m, 3H), 7.31-7.28 (m, 3H), 7.24 (d, J=7.4 Hz, 2H), 7.19 (d, J=7.1 Hz, 2H), 7.15 (s, 1H), 6.91 (d, J=2.6 Hz, 1H), 6.43 (s, 1H), 4.48 (s, 3H), 3.53 (s, 2H), 3.15-3.01 (m, 2H), 2.75-2.61 (m, 2H), 2.01-1.82 (m, 3H). LC/MS (ESI) m/z: 476 (M+H)⁺. RT (Method A): 0.94 min.

### Scheme 55. Synthesis of N-((1H-pyrrolo[3,2-c]pyridine-2-yl)methyl)-2-(5-((3-(4-((1,1-dioxidothietan-3-yl)oxy)phenyl)propyl)amino)-6-oxo-2-phenylpyrimidin-1(6H)-yl) acetamide (Compound 193)

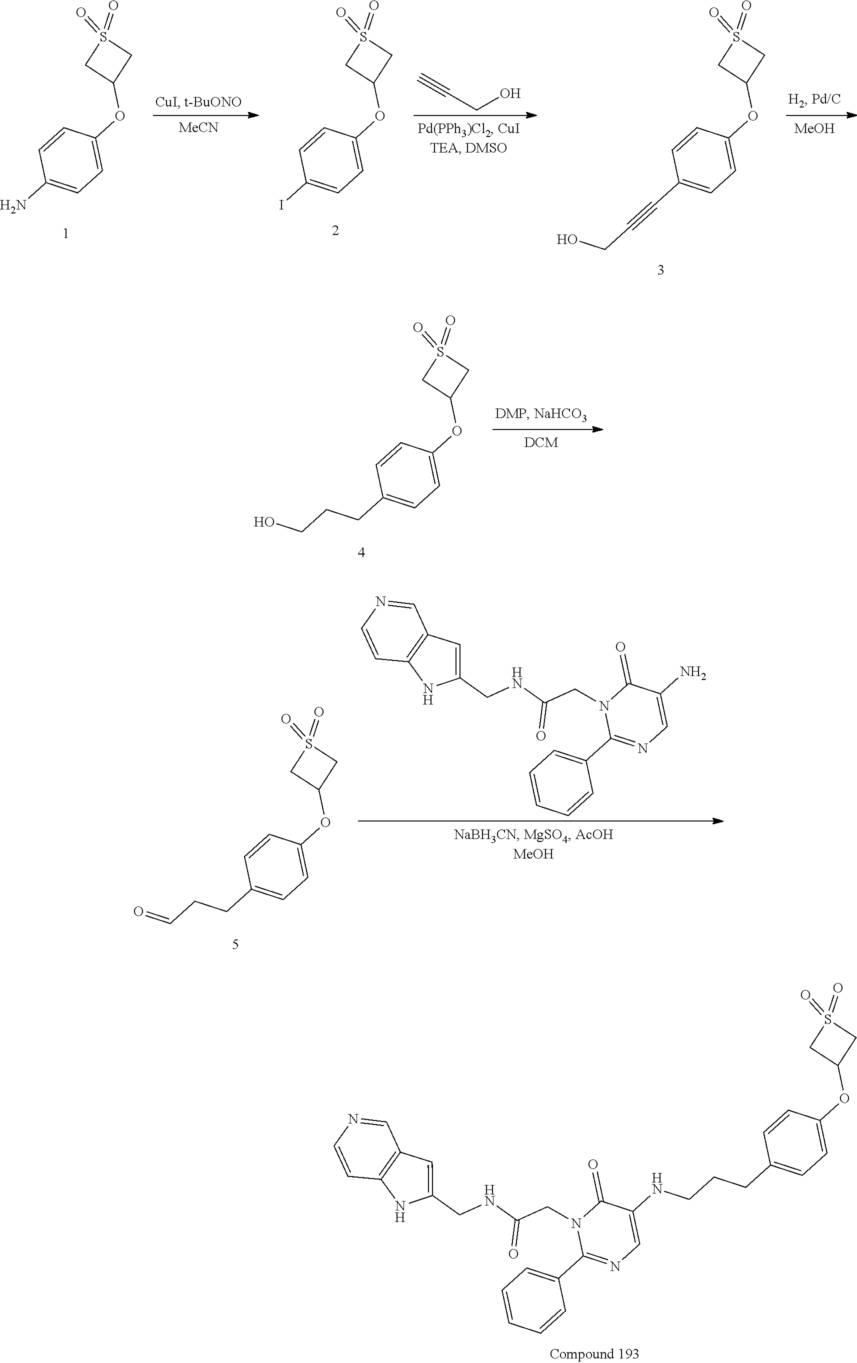

### Step 1: 3-(4-Iodophenoxy)thietane 1,1-dioxide (2)

To a solution of 3-(4-aminophenoxy)thietane 1,1-dioxide (1.3 g, 6.10 mmol) in MeCN (16 mL) was added t-BuONO (3.14 g, 30.52 mmol) at 0° C. under N₂ atmosphere and the reaction mixture was stirred at 0° C. for 5 minutes. CuI (3.49 g, 18.3 mmol) was added to the stirring mixture and the resulting mixture was stirred under N₂ atmosphere at 0° C. for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% MeOH in DCM) to give the title compound (750 mg, yield 37.9%) as a yellow solid. ¹H NMR (400 MHz, DMSO-d₆) δ 7.64 (d, J=8.7 Hz, 2H), 6.78 (d, J=8.7 Hz, 2H), 5.19-5.11 (m, 1H), 4.80-4.73 (m, 2H), 4.26-4.19 (m, 2H).

### Step 2: 3-(4-(3-Hydroxyprop-1-yn-1-yl)phenoxy)thietane 1,1-dioxide (3)

To a mixture of 3-(4-iodophenoxy)thietane 1,1-dioxide (50 mg, 0.15 mmol) and prop-2-yn-1-ol (26 mg, 0.46 mmol) in DMSO (1.4 mL) and TEA (1.4 mL) was added Pd(PPh₃)Cl₂ (11 mg, 0.015 mmol) and CuI (3 mg, 0.015 mmol) at 0° C. under N₂ atmosphere, the reaction mixture was degassed under N₂ atmosphere for three times and stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-46% EtOAc in PE) to give the title compound (37 mg, yield 95.1%) as a yellow solid. ¹H NMR (400 MHz, DMSO-d₆) δ 7.39 (d, J=8.7 Hz, 2H), 6.93 (d, J=8.7 Hz, 2H), 5.28 (t, J=5.9 Hz, 1H), 5.22-5.14 (m, 1H), 4.83-4.74 (m, 2H), 4.30-4.21 (m, 4H).

### Step 3: 3-(4-(3-Hydroxypropyl)phenoxy)thietane 1,1-dioxide (4)

To a solution of 3-(4-(3-hydroxyprop-1-yn-1-yl)phenoxy)thietane 1,1-dioxide (37 mg, 0.15 mmol) in MeOH (3 mL) was added Pd/C (10 mg, 10% wt.), the mixture was degassed under N₂ atmosphere for three times and stirred under a H₂ balloon at room temperature overnight. The mixture was filtered, and the filtrate was concentrated to dryness. The residue was purified by flash chromatography (silica gel, 0-70% EtOAc in PE) to give the title compound (24 mg, yield 63.9%) as a white solid. ¹H NMR (400 MHz, DMSO-d₆) δ 7.86 (d, J=7.0 Hz, 2H), 7.42-7.29 (m, 3H), 6.19 (s, 2H), 4.93 (t, J=5.2 Hz, 1H), 3.96 (t, J=5.9 Hz, 2H), 3.71 (q, J=5.6 Hz, 2H). LC/MS (ESI) m/z: 257 (M+H)⁺.

### Step 4: 3-(4-((1,1-Dioxidothietan-3-yl)oxy)phenyl)propanal (5)

To a solution of 3-(4-(3-hydroxypropyl)phenoxy)thietane 1,1-dioxide (23 mg, 0.090 mmol) in DCM (2 mL) was added DMP (76 mg, 0.18 mmol) and NaHCO₃ (15 mg, 0.18 mmol) at 0° C. and the mixture was stirred at room temperature for 3 hours. The reaction mixture was diluted with water and extracted with DCM twice. The combined organic layers were washed with saturated aq.NaHCO₃ solution and brine, dried over anhydrous Na₂SO₄, filtered, and concentrated under reduced pressure to dryness to give the title compound (22 mg, yield 96.4%) as a colorless oil, which was used directly in the next step without further purification. LC/MS (ESI) m/z: 255 (M+H)⁺.

### Step 5: N-((1H-pyrrolo[3,2-c]pyridine-2-yl)methyl)-2-(5-((3-(4-((1,1-dioxidothietan-3-yl)oxy)phenyl)propyl)amino)-6-oxo-2-phenylpyrimidin-1(6H)-yl)acetamide (Compound 193)

To a mixture of 3-(4-((1,1-dioxidothietan-3-yl)oxy)phenyl)propanal (22 mg, 0.087 mmol) and N-((1H-pyrrolo[3,2-c]pyridine-2-yl)methyl)-2-(5-amino-6-oxo-2-phenylpyrimidin-1(6H)-yl)acetamide (16 mg, 0.043 mmol) in MeOH (2 mL) was added MgSO₄ (16 mg, 0.13 mmol), AcOH (10 mg) and NaBH₃CN (10 mg, 0.16 mmol) successively and the reaction mixture was stirred under N₂ atmosphere at room temperature overnight. The reaction mixture was filtered, and the filtrate was concentrated under reduced pressure to dryness. The residue was purified by prep-HPLC to give Compound 193 (2.8 mg, yield 11.0%) as a white solid. ¹H NMR (400 MHz, CD₃OD) δ 8.70 (s, 1H), 8.11 (d, J=5.8 Hz, 1H), 7.46 (t, J=6.2 Hz, 3H), 7.41-7.36 (m, 3H), 7.20 (d, J=8.6 Hz, 2H), 7.05 (s, 1H), 6.85 (d, J=8.6 Hz, 2H), 6.48 (s, 1H), 5.16-5.09 (m, 1H), 4.69-4.61 (m, 4H), 4.54 (s, 2H), 4.22-4.16 (m, 2H), 3.16 (t, J=6.9 Hz, 2H), 2.71 (t, J=7.5 Hz, 2H), 2.01-1.92 (m, 2H). LC/MS (ESI) m/z: 613 (M+H)⁺. RT (Method A): 1.26 min.

### Scheme 56. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-2-(3-methoxyphenyl)oxazole-4-carboxamide (Compound 211)

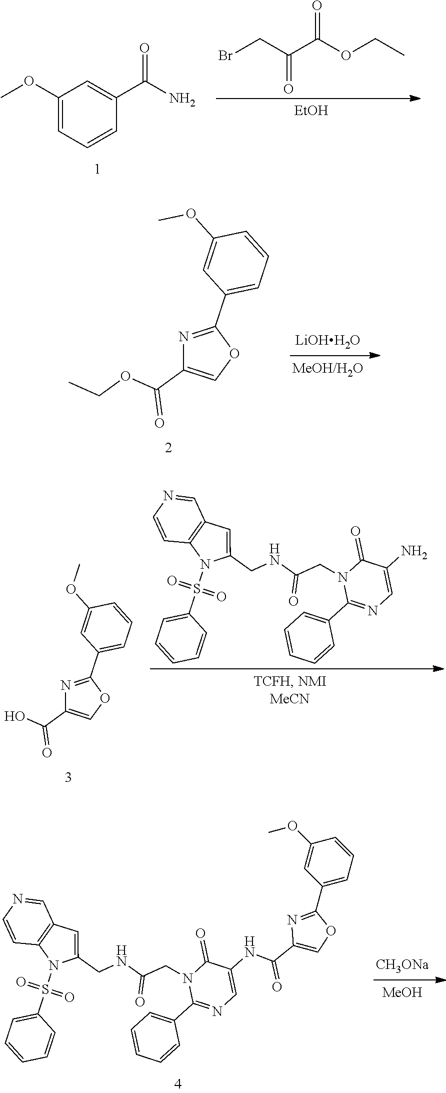

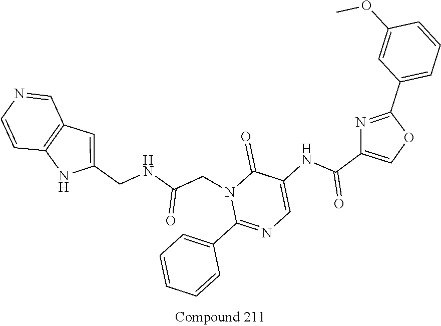 [SMILES: COc1cccc(-c2nc(C(=O)Nc3cnc(-c4ccccc4)n(CC(=O)NCc4cc5cnccc5[nH]4)c3=O)co2)c1]

### Step 1: Ethyl 2-(3-methoxyphenyl)oxazole-4-carboxylate (2)

To a mixture of 3-methoxybenzamide (500 mg, 3.31 mmol) in EtOH (8 mL) was added ethyl 3-bromo-2-oxopropanoate (1.29 g, 6.62 mmol) at 0° C. and the mixture was stirred under N₂ atmosphere at 70° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-60% EtOAc in PE) to give the title compound (302 mg, yield 36.9%) as a colorless oil. LC/MS (ESI) m/z: 248 (M+H)⁺.

### Step 2: 2-(3-Methoxyphenyl)oxazole-4-carboxylic acid (3)

To a solution of ethyl 2-(3-methoxyphenyl)oxazole-4-carboxylate (100 mg, 0.4 mmol) in MeOH/THF/water (2 mL, v/v/v=2/1/1) was added LiOH·H₂O (42 mg, 1 mmol) and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (78 mg, yield 88.9%) as a yellow solid, which was used directly in the next step without further purification. LC/MS (ESI) m/z: 220 (M+H)⁺.

### Step 3: 2-(3-methoxyphenyl)-N-(6-oxo-1-(2-oxo-2-(((1-(phenylsulfonyl)-1H-pyrrolo[3,2-c]pyridine-2-yl)methyl)amino)ethyl)-2-phenyl-1,6-dihydropyrimidin-5-yl)oxazole-4-carboxamide (4)

To a mixture of 2-(3-methoxyphenyl)oxazole-4-carboxylic acid (18 mg, 0.08 mmol) and 2-(5-amino-6-oxo-2-phenylpyrimidin-1(6H)-yl)-N-((1-(phenylsulfonyl)-1H-pyrrolo[3,2-c]pyridine-2-yl)methyl)acetamide (40 mg, 0.08 mmol) in MeCN (1 mL) was added NMI (19 mg, 0.23 mmol) and TCFH (53 mg, 0.23 mmol) at 0° C. under N₂ atmosphere and the mixture was stirred at room temperature for 1 hour. The mixture was quenched with saturated aq.NaHCO₃ solution and extracted with CHCl₃/i-PrOH (3/1, v/v) twice. The combined organic layers were washed with brine, dried over anhydrous Na₂SO₄, filtered, and concentrated under reduced pressure to dryness. The residue was purified by prep-TLC (DCM:MeOH=10:1) to give the title compound (31 mg, yield 52.7%) as a yellow solid. LC/MS (ESI) (m/z): 716 (M+H)⁺.

### Step 4: N-(1-(2-(((1H-pyrrolo[3,2-c]pyridine-2-yl)methyl)amino)-2-oxoethyl)-6-oxo-2-phenyl-1,6-dihydropyrimidin-5-yl)-2-(3-methoxyphenyl)oxazole-4-carboxamide (Compound 211)

To a solution of 2-(3-methoxyphenyl)-N-(6-oxo-1-(2-oxo-2-(((1-(phenylsulfonyl)-1H-pyrrolo[3,2-c]pyridine-2-yl)methyl)amino)ethyl)-2-phenyl-1,6-dihydropyrimidin-5-yl)oxazole-4-carboxamide (31 mg, 0.04 mmol) in MeOH (1 mL) was added MeONa (23 mg, 0.43 mmol) and the mixture was stirred at room temperature for 1 hour. The mixture was diluted with ice-water and extracted with DCM twice. The combined organic layers were washed with brine, dried over anhydrous Na₂SO₄, filtered, and concentrated under reduced pressure to dryness. The residue was purified by prep-HPLC to give Compound 211 (3.1 mg, yield 12.4%) as a white solid. ¹H NMR (400 MHz, DMSO) δ 11.36 (s, 1H), 9.47 (s, 1H), 9.03 (s, 1H), 8.98 (s, 1H), 8.84-8.78 (m, 1H), 8.75 (s, 1H), 8.11 (d, J=5.6 Hz, 1H), 7.68-7.66 (m, 1H), 7.55 (dd, J=4.4, 3.0 Hz, 5H), 7.48 (d, J=7.1 Hz, 2H), 7.33 (d, J=5.7 Hz, 1H), 7.21 (dd, J=8.0, 2.4 Hz, 1H), 6.35 (s, 1H), 4.60 (s, 2H), 4.46-4.44 (m, 2H), 3.87 (s, 3H).

### Scheme 57. Synthesis of 2-(5-((dibenzo[b,d]furan-2-ylmethyl)amino)-6-oxo-2-phenylpyrimidin-1(6H)-yl)-N-((4,5,6,7-tetrahydrothieno[3,2-c]pyridine-2-yl)methyl)acetamide (Compound 142)

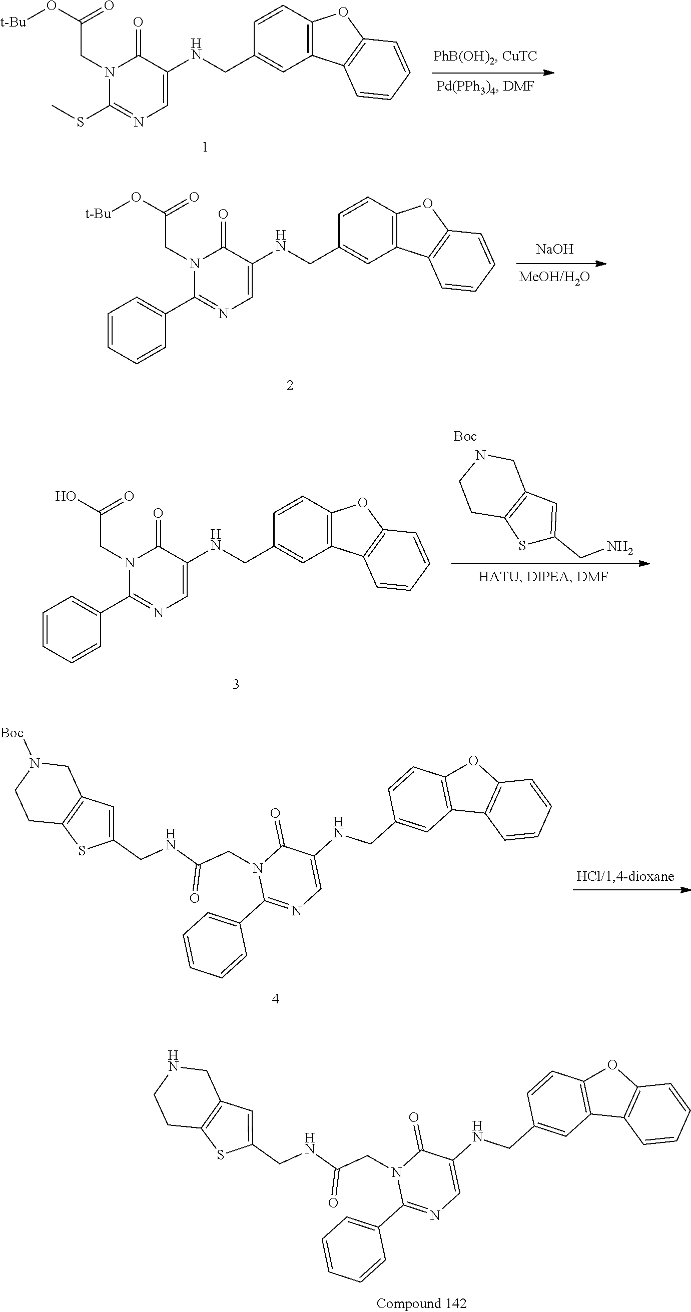

### Step 1: Tert-butyl 2-(5-((dibenzo[b,d]furan-2-ylmethyl)amino)-6-oxo-2-phenylpyrimidin-1(6H)-yl)acetate (2)

To a mixture of tert-butyl 2-(5-((dibenzo[b,d]furan-2-ylmethyl)amino)-2-(methylthio)-6-oxopyrimidin-1(6H)-yl)acetate (1.5 g, 3.3 mmol) and phenylboronic acid (198 mg, 1.3 mmol) in DMF (15 mL) was added CuTC (1.4 g, 7.3 mmol) and Pd(PPh₃)₄ (580 mg, 0.50 mmol) under N₂ atmosphere. The mixture was degassed under N₂ atmosphere, and the reaction mixture was stirred at 80° C. overnight. The mixture was diluted with EtOAc and filtered. The filtrate was washed with water and brine, dried over anhydrous Na₂SO₄, filtered, and concentrated under reduced pressure to dryness. The residue was purified by flash chromatography (silica gel, 0-30% EtOAc in PE) to give the title compound (1.3 g, yield 81.8%) as a yellow solid. LC/MS (ESI) m/z: 482 (M+H)⁺.

### Step 2: 2-(5-((Dibenzo[b,d]furan-2-ylmethyl)amino)-6-oxo-2-phenylpyrimidin-1(6H)-yl)acetic acid (3)

To a solution of tert-butyl 2-(5-((dibenzo[b,d]furan-2-ylmethyl)amino)-6-oxo-2-phenylpyrimidin-1(6H)-yl)acetate (1.3 g, 2.7 mmol) in MeOH/water (12 mL, v/v=1/1) was added NaOH (270 mg, 6.8 mmol) under N₂ atmosphere and the reaction mixture was stirred at 90° C. for 2 hours. The mixture was acidified with 1N aq. HCl to pH˜3 and extracted with EtOAc twice. The combined organic layers were dried over anhydrous Na₂SO₄, filtered, and concentrated under reduced pressure to dryness to give the title compound (1.1 g, yield 92.6%) as a yellow solid, which was used directly in the next step without further purification. LC/MS (ESI) m/z: 426 (M+H)⁺.

### Step 3: Tert-butyl 2-((2-(5-((dibenzo[b,d]furan-2-ylmethyl)amino)-6-oxo-2-phenylpyrimidin-1(6H)-yl)acetamido)methyl)-6,7-dihydrothieno[3,2-c]pyridine-5(4H)-carboxylate (4)

To a solution of 2-(5-((dibenzo[b,d]furan-2-ylmethyl)amino)-6-oxo-2-phenylpyrimidin-1(6H)-yl)acetic acid (20 mg, 0.048 mmol) in DMF (0.6 mL) was added tert-butyl 2-(aminomethyl)-6,7-dihydrothieno[3,2-c]pyridine-5(4H)-carboxylate (13 mg, 0.048 mmol), HATU (20 mg, 0.053 mmol) and DIPEA (31 mg, 0.24 mmol) and the mixture was stirred at room temperature for 10 minutes. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give the title compound (10 mg, yield 30.9%) as a white solid. LC/MS (ESI) m/z: 676 (M+H)⁺.

### Step 4: 2-(5-((dibenzo[b,d]furan-2-ylmethyl)amino)-6-oxo-2-phenylpyrimidin-1(6H)-yl)-N-((4,5,6,7-tetrahydrothieno[3,2-c]pyridine-2-yl)methyl)acetamide (Compound 142)

A solution of tert-butyl 2-((2-(5-((dibenzo[b,d]furan-2-ylmethyl)amino)-6-oxo-2-phenylpyrimidin-1(6H)-yl)acetamido)methyl)-6,7-dihydrothieno[3,2-c]pyridine-5(4H)-carboxylate (10 mg, 0.0148 mmol) in HCl/1,4-dioxane (0.5 mL, 4M) was stirred under N₂ atmosphere at room temperature for 2 hours. The reaction mixture was concentrated under reduced pressure to dryness. The residue was purified by prep-HPLC to give Compound 142 (3.5 mg, yield 41.1%) as a white solid. ¹H NMR (400 MHz, CD₃OD) δ 8.08-7.99 (m, 2H), 7.56 (t, J=6.1 Hz, 3H), 7.46 (dd, J=15.4, 10.1 Hz, 3H), 7.39 (dd, J=12.1, 4.8 Hz, 4H), 7.07 (d, J=18.6 Hz, 1H), 6.60 (s, 1H), 4.56 (s, 2H), 4.43 (s, 2H), 3.78 (s, 2H), 3.07 (t, J=5.6 Hz, 2H), 2.77 (s, 2H), 2.19 (t, J=7.5 Hz, 2H). LC/MS (ESI) m/z: 576 (M+H)⁺. RT (Method A): 1.70 min.

Compound 143 was prepared based on Steps 3-4 in Scheme 57:

| # | Reactant A | Reactant B | Characterization Data |
|---|---|---|---|
| 143 | 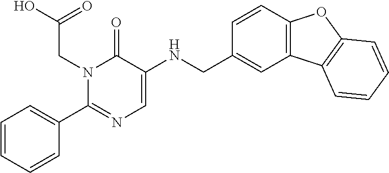 [SMILES: O=C(O)Cn1c(-c2ccccc2)ncc(NCc2ccc3oc4ccccc4c3c2)c1=O] | 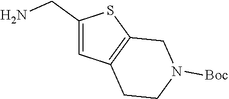 [SMILES: CC(C)(C)OC(=O)N1CCc2cc(CN)sc2C1] | ¹H NMR (400 MHz, CD₃OD) δ 8.49 (s, 1H), 8.08 (s, 1H), 8.02 (s, 1H), 7.57 (t, J = 6.6 Hz, 3H), 7.50-7.46 (m, 3H), 7.42 (t, J = 4.8 Hz, 4H), 7.36 (s, 1H), 7.11 (s, 1H), 6.74 (s, 1H), 4.58 (s, 2H), 4.57 (s, 2H), 4.46 (s, 2H), 4.29 (s, 2H), 3.40 (t, J = 6.1 Hz, 2H), 2.88 (t, J = 6.3 Hz, 2H). LC/MS (ESI) m/z: 576 (M + H)⁺. RT (Method A): 1.68 min. |

### Scheme 58. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenoxythiophene-2-carboxamide (Compound 215)

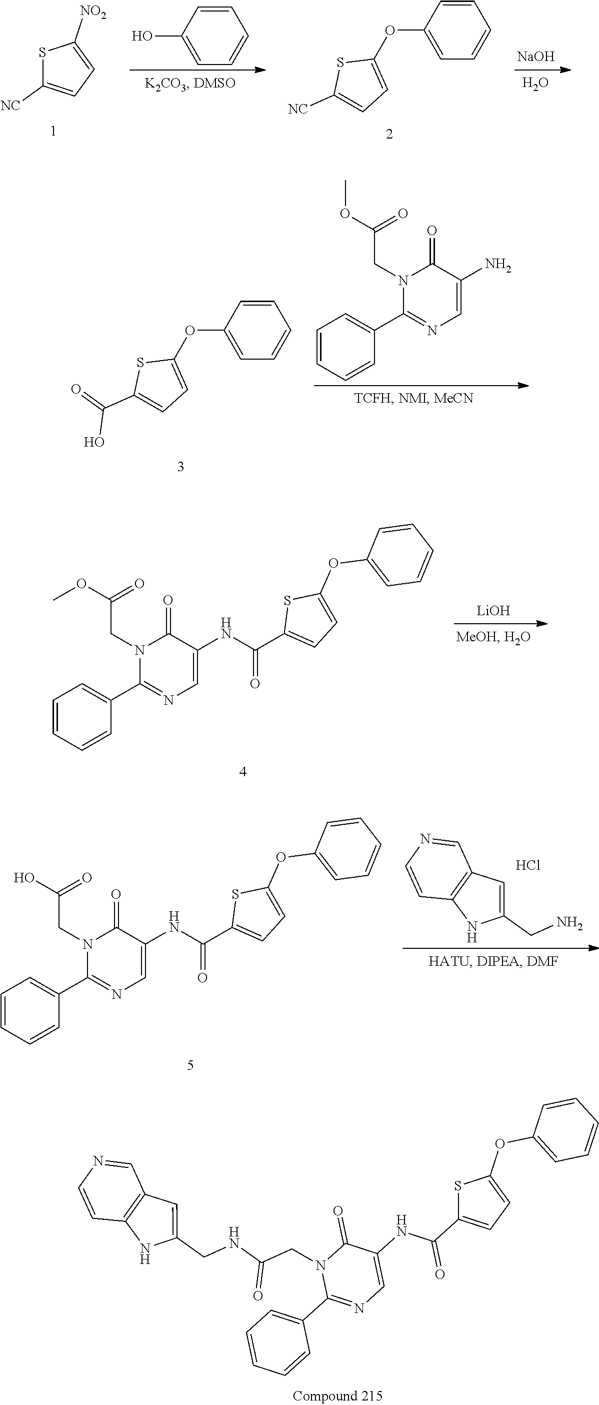

### Step 1: 5-Phenoxythiophene-2-carbonitrile (2)

To a mixture of 5-nitrothiophene-2-carbonitrile (1.0 g, 6.49 mmol) and phenol (610 mg, 6.49 mmol) in DMSO (15 mL) was added K₂CO₃ (2.7 g, 19.56 mmol) under N₂ atmosphere and the reaction mixture was stirred at 25° C. overnight. The mixture was diluted with EtOAc, washed with brine, dried over anhydrous Na₂SO₄, filtered, and concentrated under reduced pressure to dryness. The residue was purified by flash chromatography (silica gel, 100% PE) to give the title compound (560 mg, yield 42.9%) as a yellow oil. LC/MS (ESI) m/z: 202 (M+H)⁺.

### Step 2: 5-Phenoxythiophene-2-carboxylic acid (3)

To a solution of 5-phenoxythiophene-2-carbonitrile (400 mg, 1.99 mmol) in water (6 mL) was added NaOH (318 mg, 7.96 mmol) and the mixture was stirred at 100° C. overnight. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (330 mg, yield 75.4%) as a white solid, which was used directly in the next step without further purification. LC/MS (ESI) m/z: 221 (M+H)⁺.

### Step 3: Methyl 2-(6-oxo-5-(5-phenoxythiophene-2-carboxamido)-2-phenylpyrimidin-1(6H)-yl)acetate (4)

To a mixture of methyl 2-(5-amino-6-oxo-2-phenylpyrimidin-1(6H)-yl)acetate (130 mg, 0.50 mmol) and 5-phenoxythiophene-2-carboxylic acid (166 mg, 0.75 mmol) in MeCN (3 mL) was added NMI (247 mg, 3.00 mmol), TCFH (281 mg, 1.00 mmol) under N₂ atmosphere and the reaction mixture was stirred at room temperature for 2 hours. The mixture was diluted with EtOAc, washed with brine, dried over anhydrous Na₂SO₄, filtered, and concentrated under reduced pressure to dryness. The residue was purified by flash chromatography (silica gel, 0-36% EtOAc in PE) to give the title compound (180 mg, yield 77.9%) as a colorless oil. LC/MS (ESI) m/z: 462 (M+H)⁺.

### Step 4: 2-(6-Oxo-5-(5-phenoxythiophene-2-carboxamido)-2-phenylpyrimidin-1(6H)-yl)acetic acid (5)

To a solution of methyl 2-(6-oxo-5-(5-phenoxythiophene-2-carboxamido)-2-phenylpyrimidin-1(6H)-yl)acetate (180 mg, 0.39 mmol) in MeOH (3 mL) and water (1 mL) was added LiOH·H₂O (49 mg, 1.17 mmol) and the mixture was stirred at room temperature for 1 hour. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (160 mg, yield 91.9%) as a yellow solid, which was used directly in the next step without further purification. LC/MS (ESI) m/z: 448 (M+H)⁺.

### Step 5: N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenoxythiophene-2-carboxamide (Compound 215)

To a mixture of 2-(6-oxo-5-(5-phenoxythiophene-2-carboxamido)-2-phenylpyrimidin-1(6H)-yl)acetic acid (160 mg, 0.36 mmol) and (1H-pyrrolo[3,2-c]pyridine-2-yl)methanamine hydrochloride (121 mg, 0.82 mmol) in DMF (3 mL) was added DIPEA (277 mg, 2.16 mmol) and HATU (204 mg, 0.54 mmol) under N₂ atmosphere at 0° C. and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 215 (30 mg, yield 14.6%) as a white solid. ¹H NMR (400 MHz, CD₃OD) δ 8.90 (s, 1H), 8.72 (s, 1H), 8.11 (d, J=5.8 Hz, 1H), 7.70 (d, J=4.2 Hz, 1H), 7.55 (d, J=7.4 Hz, 2H), 7.52 (d, J=7.1 Hz, 1H), 7.46 (d, J=7.6 Hz, 2H), 7.43 (d, J=7.3 Hz, 2H), 7.39 (d, J=5.9 Hz, 1H), 7.27 (d, J=7.3 Hz, 1H), 7.22 (d, J=8.6 Hz, 2H), 6.57 (d, J=4.2 Hz, 1H), 6.49 (s, 1H), 4.70 (s, 2H), 4.55 (s, 2H). LC/MS (ESI) (m/z): 577 (M+H)⁺.

Compound 203 was prepared based on Steps 2-5 in Scheme 58:

| # | Reactant A | Reactant C | Reactant D | Characterization Data |
|---|---|---|---|---|
| 203^{a} | 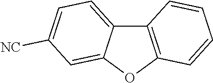 [SMILES: N#Cc1ccc2c(c1)oc1ccccc12] | 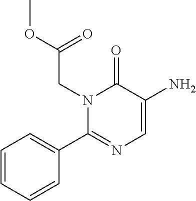 | 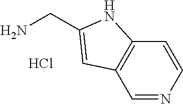 [SMILES: NCc1cc2cnccc2[nH]1 ; Cl] | ¹H NMR (400 MHz, DMSO-d₆) δ 11.40 (s, 1H), 9.74 (s, 1H), 8.83 (s, 1H), 8.81-8.78 (m, 1H), 8.78-8.70 (m, 1H), 8.34-8.33 (m, 1H), 8.33-8.30 (m, 1H), 8.26 (d, J = 7.7 Hz, 1H), 8.15-8.13 (m, 1H), 8.05 (dd, J = 8.2, 1.3 Hz, 1H), 7.82-7.78 (m, 1H), 7.65-7.60 (m, 1H), 7.60-7.58 (m, 1H), 7.58-7.57 (m, 1H), 7.57-7.52 (m, 1H), 7.51-7.49 (m, 1H), 7.49-7.48 (m, 1H), 7.47-7.44 (m, 1H), 7.38-7.33 (m, 1H), 6.35 (s, 1H), 4.61 (s, 2H), 4.44 (d, J = 5.5 Hz, 2H). LC/MS (ESI) m/z: 569 (M + H)⁺. RT (Method A): 1.54 min. |

^{a}NaOH was used in place of KOH in Step 2.

### Scheme 59. Synthesis of N-((1H-pyrrolo[3,2-c]pyridine-2-yl)methyl)-2-(5-(isoindolin-2-yl)-6-oxo-2-phenylpyrimidin-1(6H)-yl)acetamide (Compound 184)

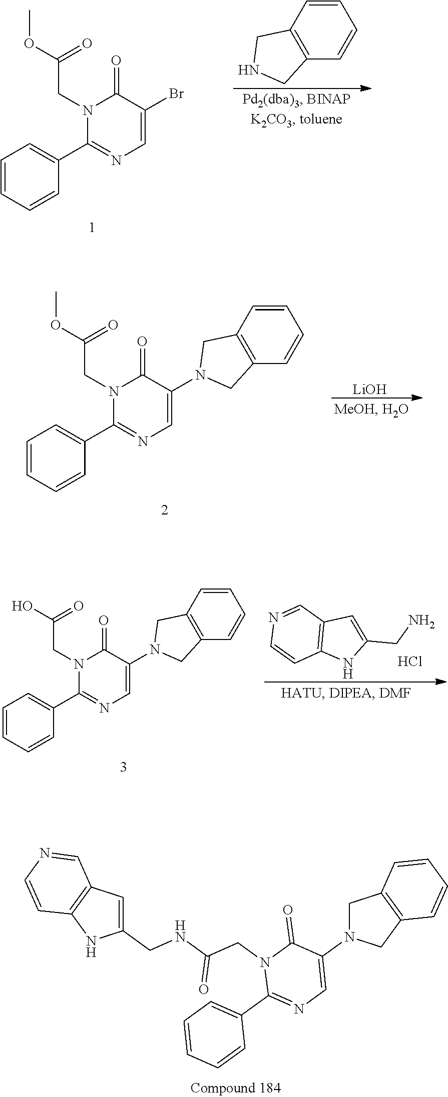

### Step 1: Methyl 2-(5-(isoindolin-2-yl)-6-oxo-2-phenylpyrimidin-1(6H)-yl)acetate (2)

To a solution of methyl 2-(5-bromo-6-oxo-2-phenylpyrimidin-1(6H)-yl)acetate (100 mg, 0.31 mmol) in toluene (5 mL) was added isoindoline (74 mg, 0.62 mmol), Cs₂CO₃ (306 mg, 0.93 mmol), BINAP (19 mg, 0.031 mmol) and Pd₂(dba)₃ (151 mg, 0.031 mmol) under N₂ atmosphere, the reaction mixture was degassed under N₂ atmosphere for three times and stirred at 80° C. for 2 hours. The mixture was diluted EtOAc, washed with water and brine, dried over anhydrous Na₂SO₄, filtered, and concentrated under reduced pressure to dryness. The residue was purified by flash chromatography (silica gel, 0-15% EtOAc in PE) to give the title compound (90 mg, yield 80.4%) as a white solid. LC/MS (ESI) m/z: 362 (M+H)⁺.

### Step 2: 2-(5-(isoindolin-2-yl)-6-oxo-2-phenylpyrimidin-1(6H)-yl)acetic acid (3)

To a solution of methyl 2-(5-(isoindolin-2-yl)-6-oxo-2-phenylpyrimidin-1(6H)-yl)acetate (90 mg, 0.25 mmol) in MeOH/H₂O (4 mL, v/v=3/1) was added LiOH (21 mg, 0.50 mmol) and the mixture was stirred at room temperature for 2 hours. The mixture was acidified with 1 N aq. HCl to pH˜6 and extracted with EtOAc twice. The combined organic layers were washed with brine, dried over anhydrous Na₂SO₄, filtered, and concentrated under reduced pressure to dryness to give the title compound (80 mg, yield 92.0%) as a white solid, which was used directly in the next step without further purification. LC/MS (ESI) m/z: 348 (M+H)⁺.

### Step 3: N-((1H-pyrrolo[3,2-c]pyridine-2-yl)methyl)-2-(5-(isoindolin-2-yl)-6-oxo-2-phenylpyrimidin-1(6H)-yl)acetamide (Compound 184)

To a mixture of 2-(5-(isoindolin-2-yl)-6-oxo-2-phenylpyrimidin-1(6H)-yl)acetic acid (87 mg, 0.25 mmol) and (1H-pyrrolo[3,2-c]pyridine-2-yl)methanamine hydrochloride (74 mg, 0.50 mmol) in DMF (3 mL) was added DIPEA (194 mg, 1.50 mmol), HATU (143 mg, 0.38 mmol) under N₂ atmosphere and the reaction mixture was stirred at room temperature for 2 hours. The mixture was diluted with EtOAc, washed with saturated aq.NH₄Cl solution, dried over anhydrous Na₂SO₄, filtered, and concentrated under reduced pressure to dryness. The residue was purified by flash chromatography (silica gel, 0-10% MeOH in DCM) and further purified by prep-HPLC to give the title compound (15 mg, yield 12.5%) as a white solid. ¹H NMR (400 MHz, DMSO-d₆) δ 11.33 (s, 1H), 8.77 (t, J=5.8 Hz, 1H), 8.42 (s, 1H), 8.27-8.23 (m, 2H), 8.08-8.05 (m, 2H), 7.42-7.36 (m, 5H), 7.33-7.30 (m, 2H), 7.25 (d, J=5.6 Hz, 1H), 6.32 (s, 1H), 5.08 (s, 2H), 4.93 (s, 4H), 4.50 (d, J=5.7 Hz, 2H). LC/MS (ESI) m/z: 477 (M+H)⁺.

### Scheme 60. Synthesis of N-((1H-pyrrolo[3,2-c]pyridine-2-yl)methyl)-2-(5-((dibenzo[b,d]furan-2-ylmethyl)amino)-2-(4-(oxetan-3-yloxy)phenyl)-6-oxopyrimidin-1(6H)-yl)acetamide (Compound 130)

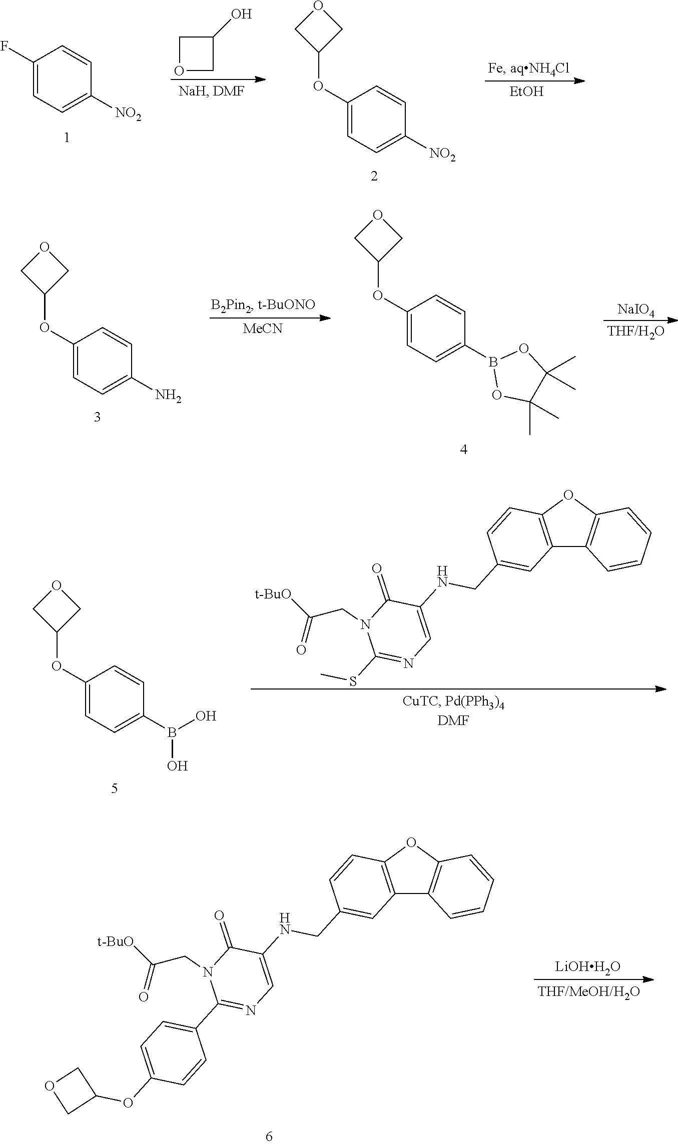

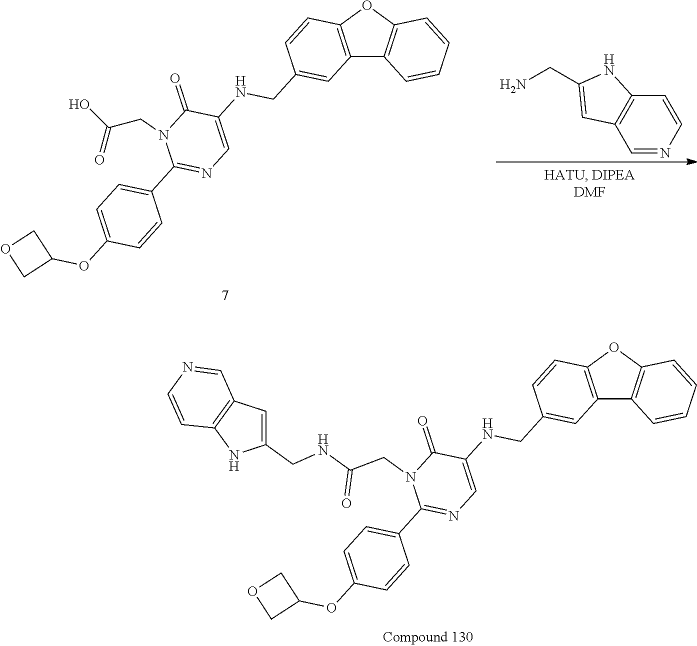 [SMILES: O=C(O)Cn1c(-c2ccc(OC3COC3)cc2)ncc(NCc2ccc3oc4ccccc4c3c2)c1=O ; NCc1cc2cnccc2[nH]1 ; O=C(Cn1c(-c2ccc(OC3COC3)cc2)ncc(NCc2ccc3oc4ccccc4c3c2)c1=O)NCc1cc2cnccc2[nH]1]

### Step 1: 3-(4-Nitrophenoxy)oxetane (2)

To a solution of oxetan-3-ol (1.8 g, 24.30 mmol) in DMF (20 mL) was added NaH (1.2 g, 29.16 mmol, 60% dispersion in mineral oil) under N₂ atmosphere and the reaction mixture was stirred at 0° C. for 30 minutes. Then 1-fluoro-4-nitrobenzene (5.1 g, 36.45 mmol) was added into the above mixture and the reaction mixture was stirred at room temperature for 2 hours. The reaction mixture was quenched with saturated aq.NH₄Cl solution and extracted with EtOAc twice. The combined organic layers were washed with water and brine, dried over anhydrous Na₂SO₄, filtered, and concentrated under reduced pressure to dryness. The residue was purified by flash chromatography (silica gel, 0-10% EtOAc in PE) to give the title compound (1.8 g, yield 38.0%) as a white solid. ¹H NMR (400 MHz, CDCl₃) δ 8.21-8.12 (m, 2H), 6.78-6.71 (m, 2H), 5.32-5.26 (m, 1H), 4.99 (t, J=6.9 Hz, 2H), 4.74 (dd, J=7.6, 5.3 Hz, 2H).

### Step 2: 4-(Oxetan-3-yloxy)aniline (3)

To a solution of 3-(4-nitrophenoxy)oxetane (500 mg, 2.56 mmol) in EtOH (5 mL) and saturated aq.NH₄Cl solution (5 mL) was added Fe (717 mg, 12.81 mmol) under N₂ atmosphere, and the reaction mixture was stirred at 80° C. overnight. The mixture was diluted with EtOAc and filtered. The filtrate was washed with water and brine, dried over anhydrous Na₂SO₄, filtered, and concentrated under reduced pressure to dryness. The residue was purified by flash chromatography (silica gel, 40-60% EtOAc in PE) to give the title compound (400 mg, yield 94.5%) as a white solid. ¹H NMR (400 MHz, DMSO-d₆) δ 6.49 (s, 4H), 5.12-5.02 (m, 1H), 4.83 (t, J=6.6 Hz, 2H), 4.65 (s, 2H), 4.52-4.46 (m, 2H).

### Step 3: 4,4,5,5-Tetramethyl-2-(4-(oxetan-3-yloxy)phenyl)-1,3,2-dioxaborolane (4)

To a solution of 4-(oxetan-3-yloxy)aniline (100 mg, 0.61 mmol) in MeCN (5 mL) was added t-BuONO (312 mg, 3.03 mmol) under N₂ atmosphere and the reaction mixture was stirred at 0° C. for 10 minutes. Then 4,4,4′,4′,5,5,5′,5′-octamethyl-2,2′-bi(1,3,2-dioxaborolane) (461 mg, 1.82 mmol) was added into the above mixture and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% EtOAc in PE) to give the title compound (160 mg, yield 95.7%) as a white solid. ¹H NMR (400 MHz, CDCl₃) δ 7.73 (d, J=8.4 Hz, 2H), 6.68 (d, J=8.4 Hz, 2H), 5.26-5.18 (m, 1H), 4.97 (t, J=6.7 Hz, 2H), 4.80-4.69 (m, 2H), 1.32 (s, 12H), 1.24 (d, J=8.0 Hz, 39H).

### Step 4: (4-(Oxetan-3-yloxy)phenyl)boronic acid (5)

To a solution of 4,4,5,5-tetramethyl-2-(4-(oxetan-3-yloxy)phenyl)-1,3,2-dioxaborolane (160 mg, 0.58 mmol) in THF (5 mL) and water (5 mL) was added NaIO₄ (248 mg, 1.16 mmol) under N₂ atmosphere and the reaction mixture was stirred at 50° C. overnight. The mixture was diluted with EtOAc, washed with water and brine, dried over anhydrous Na₂SO₄, filtered, and concentrated under reduced pressure to dryness. The residue was purified by flash chromatography (silica gel, 40-60% EtOAc in PE) to give the title compound (90 mg, yield 80.1%) as the white solid. ¹H NMR (400 MHz, DMSO-d₆) δ 7.88 (s, 1H), 7.66 (dd, J=49.0, 8.3 Hz, 2H), 6.75 (dd, J=20.1, 8.3 Hz, 2H), 5.28 (dd, J=11.0, 5.6 Hz, 1H), 4.92 (t, J=6.6 Hz, 2H), 4.56-4.47 (m, 2H).

### Step 5: Tert-butyl 2-(5-((dibenzo[b,d]furan-2-ylmethyl)amino)-2-(4-(oxetan-3-yloxy) phenyl)-6-oxopyrimidin-1(6H)-yl)acetate (6)

To a mixture of tert-butyl 2-(5-((dibenzo[b,d]furan-2-ylmethyl)amino)-2-(methylthio)-6-oxopyrimidin-1(6H)-yl)acetate (105 mg, 0.23 mmol) and (4-(oxetan-3-yloxy)phenyl)boronic acid (90 mg, 0.46 mmol) in DMF (5 mL) was added CuTC (97 mg, 0.51 mmol) and Pd(PPh₃)₄ (27 mg, 0.023 mmol) under N₂ atmosphere and the reaction mixture was stirred at 80° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give the title compound (40 mg, yield 31.1%) as a white solid. LC/MS (ESI) m/z: 554 (M+H)⁺.

### Step 6: 2-(5-((Dibenzo[b,d]furan-2-ylmethyl)amino)-2-(4-(oxetan-3-yloxy)phenyl)-6-oxopyrimidin-1(6H)-yl)acetic acid (7)

To a solution of tert-butyl 2-(5-((dibenzo[b,d]furan-2-ylmethyl)amino)-2-(4-(oxetan-3-yloxy)phenyl)-6-oxopyrimidin-1(6H)-yl)acetate (40 mg, 0.072 mmol) in THF/MeOH/water (3 mL, v/v/v=4/1/1) was added LiOH H₂O (6 mg, 0.14 mmol) under N₂ atmosphere and the reaction mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (30 mg, yield 83.5%) as a white solid, which was used directly in the next step without further purification. LC/MS (ESI) m/z: 498 (M+H)⁺.

### Step 7: N-((1H-pyrrolo[3,2-c]pyridine-2-yl)methyl)-2-(5-((dibenzo[b,d]furan-2-ylmethyl)amino)-2-(4-(oxetan-3-yloxy)phenyl)-6-oxopyrimidin-1(6H)-yl)acetamide (Compound 130)

To a mixture of 2-(5-((dibenzo[b,d]furan-2-ylmethyl)amino)-2-(4-(oxetan-3-yloxy)phenyl)-6-oxopyrimidin-1(6H)-yl)acetic acid (30 mg, 0.060 mmol) and (1H-pyrrolo[3,2-c]pyridine-2-yl)methanamine hydrochloride (17 mg, 0.090 mmol) in DMF (3 mL) was added HATU (25 mg, 0.066 mmol) and DIPEA (47 mg, 0.36 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (DCM:MeOH=5:1) and further purified by prep-HPLC to give Compound 130 (4 mg, yield 10.6%) as a white solid. ¹H NMR (400 MHz, DMSO) δ 11.32 (s, 1H), 8.78-8.66 (m, 2H), 8.18-8.08 (m, 3H), 7.73-7.64 (m, 2H), 7.59-7.45 (m, 2H), 7.42-7.33 (m, 4H), 7.06 (d, J=14.9 Hz, 1H), 6.72 (d, J=8.7 Hz, 2H), 6.39-6.24 (m, 2H), 5.25-5.16 (m, 1H), 4.87 (t, J=6.6 Hz, 2H), 4.54-4.46 (m, 6H), 4.43 (d, J=5.3 Hz, 2H). LC/MS (ESI) m/z: 627 (M+H)⁺.

Compound 153 was prepared based on Steps 3-7 of Scheme 60:

| # | Reactant A | Reactant B | Reactant C | Characterization Data |
|---|---|---|---|---|
| 153a | 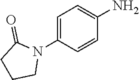 [SMILES: Nc1ccc(N2CCCC2=O)cc1] | 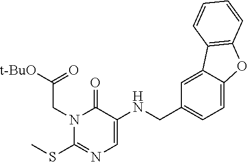 | 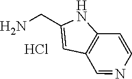 [SMILES: NCc1cc2cnccc2[nH]1 ; Cl] | ¹H NMR (400 MHz, DMSO-d₆) δ 14.55 (s, 1H), 12.62 (s, 1H), 9.19 (s, 1H), 8.87 (s, 1H), 8.39 (d, J = 7.0 Hz, 1H), 8.17 (s, 1H), 8.11 (d, J = 7.4 Hz, 1H), 7.93 (d, J = 6.7 Hz, 1H), 7.69 (dd, J = 8.2, 6.7 Hz, 2H), 7.62 (d, J = 8.8 Hz, 2H), 7.57 (m, 1H), 7.51 (d, J = 7.1 Hz, 1H), 7.40 (d, J = 8.6 Hz, 2H), 7.10 (m, 1H), 6.80 (s, 1H), 6.30 (t, J = 6.4 Hz, 1H), 4.56-4.49 (m, 6H), 3.78 (t, J = 7.1 Hz, 2H), 2.53 (t, 2H), 2.07 (dd, J = 14.9, 7.5 Hz, 2H). LC/MS (ESI) m/z: 638 (M + H)⁺. RT (Method A): 1.57 min. |

### Scheme 61. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyloxazole-2-carboxamide (Compound 171)

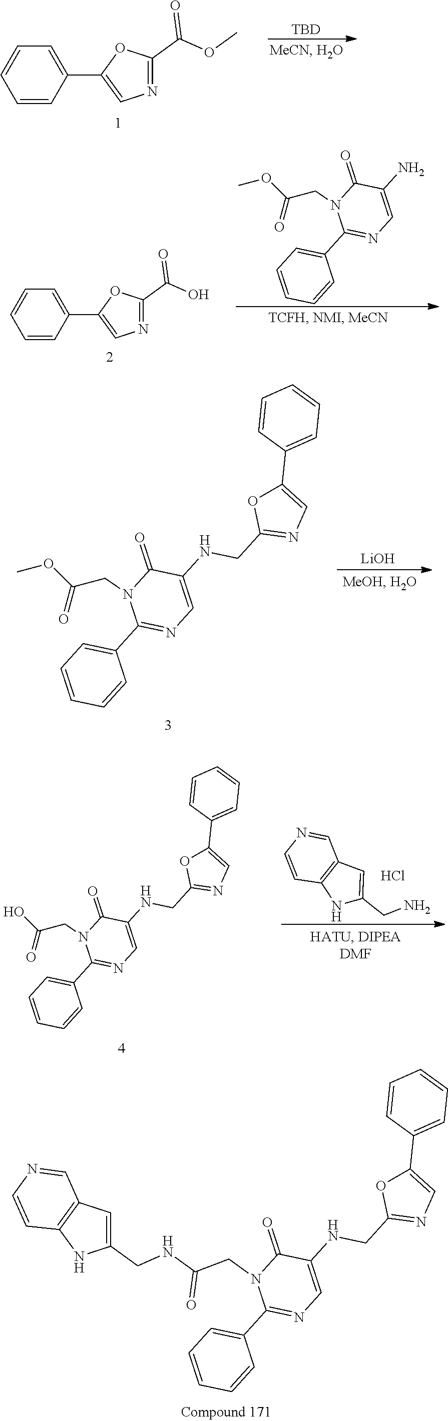

### Step 1: 5-Phenyloxazole-2-carboxylic acid (2)

To a solution of ethyl 5-phenyloxazole-2-carboxylate (120 mg, 0.55 mmol) in MeCN/H₂O (1.25 mL, 4/1) was added 1,5,7-triazabicyclo[4.4.0]dec-5-ene (115 mg, 0.83 mmol) and the mixture was stirred at room temperature for 2 hours. The mixture was concentrated under reduced pressure to dryness to give the title compound (140 mg, crude) as a yellow oil, which was used directly in the next step without further purification. LC/MS (ESI) m/z: 190 (M+H)⁺.

### Step 2: Methyl 2-(6-oxo-2-phenyl-5-(5-phenyloxazole-2-carboxamido)pyrimidin-1(6H)-yl)acetate (3)

To a mixture of methyl 2-(5-amino-6-oxo-2-phenylpyrimidin-1(6H)-yl)acetate (120 mg, 0.46 mmol) and 5-phenyloxazole-2-carboxylic acid (114 mg, 0.60 mmol) in MeCN (2 mL) was added NMI (113 mg, 1.38 mmol) and TCFH (387 mg, 1.38 mmol) and the mixture was stirred at room temperature for 1 hour. The mixture was diluted with DCM, washed with saturated aq.NaHCO₃ solution and brine, dried over anhydrous Na₂SO₄, filtered, and concentrated under reduced pressure to dryness. The residue was purified by flash chromatography (silica gel, 0-5% MeOH in DCM) to give the title compound (155 mg, yield 78.3%) as a white solid. LC/MS (ESI) m/z: 431 (M+H)⁺.

### Step 3: 2-(6-Oxo-2-phenyl-5-(5-phenyloxazole-2-carboxamido)pyrimidin-1(6H)-yl)acetic acid (4)

To a solution of methyl 2-(6-oxo-2-phenyl-5-(5-phenyloxazole-2-carboxamido) pyrimidin-1(6H)-yl)acetate (155 mg, 0.36 mmol) in MeOH/water (1.25 mL, v/v=4/1) was added LiOH (30 mg, 0.72 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (120 mg, yield 80.0%) as a white solid, which was used directly in the next step without further purification. LC/MS (ESI) m/z: 417 (M+H)⁺.

### Step 5: N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyloxazole-2-carboxamide (Compound 171)

To a mixture of 2-(6-oxo-2-phenyl-5-(5-phenyloxazole-2-carboxamido)pyrimidin-1(6H)-yl)acetic acid (120 mg, 0.29 mmol) and (1H-pyrrolo[3,2-c]pyridine-2-yl)methanamine hydrochloride (85 mg, 0.58 mmol) in DMF (2 mL) was added DIPEA (186 mg, 1.44 mmol) and HATU (164 mg, 0.43 mmol) and the reaction mixture was stirred at room temperature for 2 hours. The mixture was concentrated under reduced pressure to dryness. The residue was purified by prep-HPLC to give Compound 171 (23.4 mg, yield 14.9%) as a white solid. ¹H NMR (400 MHz, DMSO-d₆) δ 12.62 (s, 1H), 9.61 (s, 1H), 9.21 (s, 1H), 8.94 (t, J=5.7 Hz, 1H), 8.88 (s, 1H), 8.38 (d, J=6.6 Hz, 1H), 8.05 (s, 1H), 7.91 (d, J=6.6 Hz, 1H), 7.87 (d, J=7.4 Hz, 2H), 7.59-7.55 (m, 5H), 7.50-7.45 (m, 3H), 6.76 (s, 1H), 4.64 (s, 2H), 4.54 (d, J=5.6 Hz, 2H). LC/MS (ESI) m/z: 546 (M+H)⁺. RT (Method A): 1.35 min.

### Scheme 62. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-2-fluoro-3′-methoxy-[1,1′-biphenyl]-4-carboxamide (Compound 217)

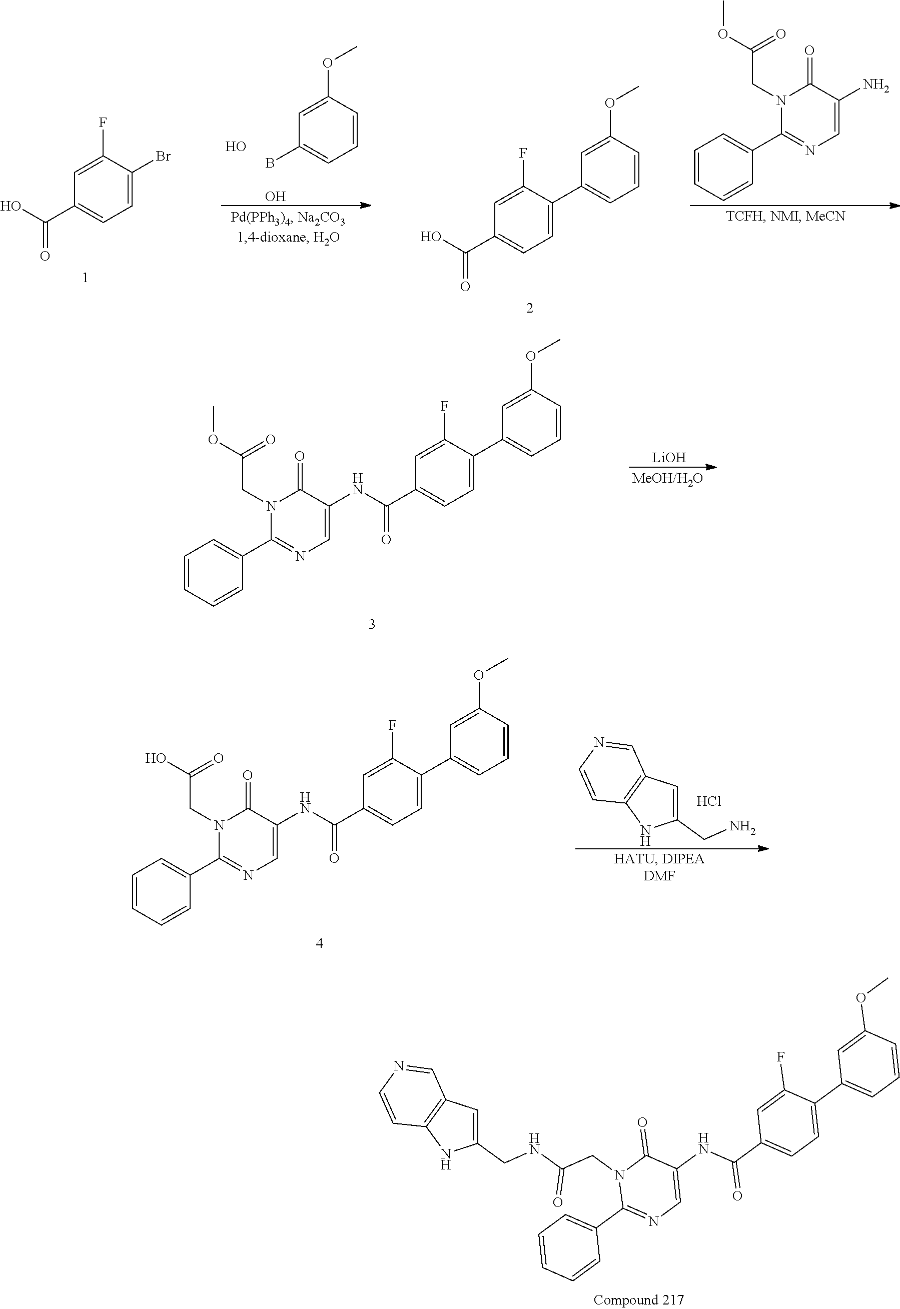

### Step 1: 2-Fluoro-3′-methoxy-[1,1′-biphenyl]-4-carboxylic acid (2)

To a solution of 4-bromo-3-fluorobenzoic acid (500 mg, 2.28 mmol) and (3-methoxyphenyl)boronic acid (382 mg, 2.51 mmol) in 1,4-dioxane (5 mL) and water (2 mL) was added Pd(PPh₃)₄ (264 mg, 0.23 mmol), Na₂CO₃ (739 mg, 6.84 mmol) at 25° C. under N₂ atmosphere, the reaction mixture was degassed under N₂ atmosphere for three times and stirred under N₂ atmosphere at 80° C. overnight. The mixture was diluted with water and extracted with EtOAc twice. The combined organic layers were washed with brine, dried over anhydrous Na₂SO₄, filtered and concentrated under reduced pressure to dryness. The residue was purified by flash chromatography (silica gel, 0-30% EtOAc in PE) to give the title compound (240 mg, yield 42.8%) as a colorless oil. LC/MS (ESI) m/z: 247 (M+H)⁺.

### Step 2: Methyl 2-(5-(2-fluoro-3′-methoxy-[1,1′-biphenyl]-4-carboxamido)-6-oxo-2-phenylpyrimidin-1(6H)-yl)acetate (4)

To a mixture of 2-fluoro-3′-methoxy-[1,1′-biphenyl]-4-carboxylic acid (160 mg, 0.65 mmol) and methyl 2-(5-amino-6-oxo-2-phenylpyrimidin-1(6H)-yl)acetate (120 mg, 0.46 mmol) in MeCN (2 mL) was added NMI (115 mg, 1.39 mmol), TCFH (390 mg, 1.39 mmol) and the mixture was stirred at room temperature for 5 hours. The mixture was diluted with EtOAc, washed with water and brine, dried over anhydrous Na₂SO₄, filtered and concentrated under reduced pressure to dryness. The residue was purified by flash chromatography (silica gel, 0-5% MeOH in DCM) to give the title compound (173 mg, yield 76.9%) as a yellow oil. LC/MS (ESI) m/z: 488 (M+H)⁺.

### Step 3: 2-(5-(2-Fluoro-3′-methoxy-[1,1′-biphenyl]-4-carboxamido)-6-oxo-2-phenylpyrimidin-1(6H)-yl)acetic acid (5)

To a solution of methyl 2-(5-(2-fluoro-3′-methoxy-[1,1′-biphenyl]-4-carboxamido)-6-oxo-2-phenylpyrimidin-1(6H)-yl)acetate (170 mg, 0.35 mol) in MeOH (4 mL) and water (2 mL) was added LiOH (22 mg, 0.52 mol) and the mixture was stirred at 25° C. for 5 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (80 mg, yield 48.5%) as a yellow oil. LC/MS (ESI) m/z: 474 (M+H)⁺.

### Step 4: N-(1-(2-(((1H-pyrrolo[3,2-c]pyridine-2-yl)methyl)amino)-2-oxoethyl)-6-oxo-2-phenyl-1,6-dihydropyrimidin-5-yl)-2-fluoro-3′-methoxy-[1,1′-biphenyl]-4-carboxamide (Compound 217)

To a mixture of 2-(5-(2-fluoro-3′-methoxy-[1,1′-biphenyl]-4-carboxamido)-6-oxo-2-phenylpyrimidin-1(6H)-yl)acetic acid (80 mg, 0.17 mmol) and (1H-pyrrolo[3,2-c]pyridine-2-yl)methanamine hydrochloride (38 mg, 0.20 mmol) in DMF (3 mL) was added DIPEA (66 mg, 0.51 mmol), HATU (97 mg, 0.25 mol) and the mixture was stirred at room temperature for 6 hours. The mixture was diluted with water and extracted with EtOAc twice. The combined organic layer was washed with brine, dried over anhydrous Na₂SO₄, filtered, and concentrated under reduced pressure to dryness. The residue was purified by prep-TLC (DCM:MeOH=10:1) and further purified by prep-HPLC to give Compound 217 (22.4 mg, yield 22.2%) as a white solid. ¹H NMR (400 MHz, DMSO-d₆) δ 9.75 (s, 1H), 9.17 (s, 1H), 8.93 (t, J=5.7 Hz, 1H), 8.78 (s, 1H), 8.37 (d, J=6.6 Hz, 1H), 7.96-7.83 (m, 3H), 7.74 (t, J=8.0 Hz, 1H), 7.64-7.52 (m, 3H), 7.51-7.39 (m, 3H), 7.18 (d, J=18.2 Hz, 2H), 7.08-6.99 (m, 1H), 6.74 (s, 1H), 4.63 (s, 2H), 4.53 (d, J=5.5 Hz, 2H), 3.83 (s, 3H). LC/MS (ESI) m/z: 603 (M+H)⁺. RT (Method A): 1.64 min.

The following compounds were prepared based on Scheme 62:

| # | Reactant A | Reactant B | Reactant C | Reactant D | Characterization Data |
|---|---|---|---|---|---|
| 168^{a} | 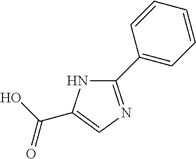 [SMILES: O=C(O)c1cnc(-c2ccccc2)[nH]1] | | 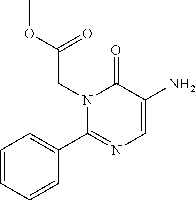 | 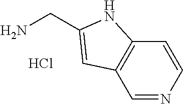 [SMILES: NCc1cc2cnccc2[nH]1 ; Cl] | ¹H NMR (400 MHz, DMSO-d₆) δ 13.32 (s, 1H), 11.41 (s, 1H), 9.69 (s, 1H), 9.01 (s, 1H), 8.81 (m, 1H), 8.78 (s, 1H), 8.15 (s, 1H), 8.11 (d, J = 5.7 Hz, 1H), 8.05 (s, 2H), 8.03 (s, 1H), 7.55 (s, 2H), 7.53 (d, J = 7.7 Hz, 2H), 7.49-7.44 (m, 3H), 7.36 (d, J = 5.7 Hz, 1H), 6.39 (s, 1H), 4.60 (s, 2H), 4.46 (d, J = 5.5 Hz, 2H). LCMS (ESI) (m/z): 545 (M + H)⁺. RT |
| | | | | | (Method A): 1.01 min. |
| 177^{a,c} | 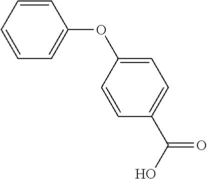 [SMILES: O=C(O)c1ccc(Oc2ccccc2)cc1] | | 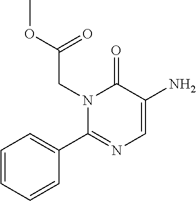 | 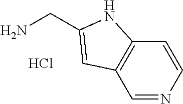 [SMILES: NCc1cc2cnccc2[nH]1 ; Cl] | ¹H NMR (400 MHz, DMSO-d₆) δ11.35 (s, 1H), 10.14 (s, 1H), 8.81-8.64 (m, 2H), 8.11 (d, J = 5.7 Hz, 1H), 8.00 (d, J = 8.8 Hz, 2H), 7.54-7.43 (m, 7H), 7.32 (d, J = 5.6 Hz, 1H), 7.24 (t, J = 7.4 Hz, 1H), 7.17-7.06 (m, 5H), 6.31 (s, 1H), 4.49 (s, 2H), 4.41 (d, J = 5.5 Hz, 2H). LC/MS (ESI) m/z: 571 (M + H)⁺. RT (Method A): 1.46 min. |
| 179ª | 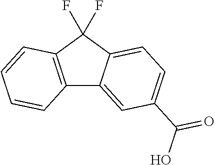 [SMILES: O=C(O)c1ccc2c(c1)-c1ccccc1C2(F)F] | | 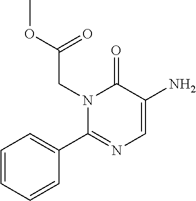 | 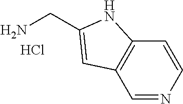 [SMILES: NCc1cc2cnccc2[nH]1 ; Cl] | ¹H NMR (400 MHz, DMSO-d₆) δ 11.40 (s, 1H), 10.50 (m, 1H), 8.79-8.73 (m, 2H), 8.36 (m, 1H), 8.23- 8.04 (m, 2H), 7.94 (m, 2H), 7.79 (t, J = 7.9 Hz, 1H), 7.66 (dd, J = 12.9, 7.4 Hz, 1H), 7.50 (m, 6H), 7.35 (d, J = 5.7 Hz, 1H), 7.16 (d, J = 2.2 Hz, 1H), 6.34 (s, 1H), 4.52 (s, 2H), 4.43 (d, J = 5.5 Hz, 2H). LC/MS (ESI) m/z: 603 (M + H)⁺. RT (Method A): 1.59 min. |
| 204^{a} | 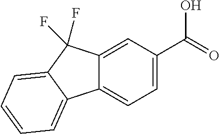 [SMILES: O=C(O)c1ccc2c(c1)C(F)(F)c1ccccc1-2] | | 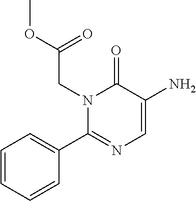 | 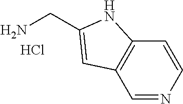 [SMILES: NCc1cc2cnccc2[nH]1 ; Cl] | ¹H NMR (400 MHz, CD₃OD) δ 9.04 (s, 1H), 8.91 (s, 1H), 8.22 (d, J = 6.5 Hz, 2H), 8.19 - 8.16 (m, 1H), 7.91 (d, J = 8.1 Hz, 1H), 7.84 (d, J = 7.6 Hz, 1H), 7.69 (d, J = 6.7 Hz, 2H), 7.61-7.57 (m, 3H), 7.56-7.53 (m, 1H), 7.51-7.46 (m, 3H), 6.75 (s, 1H), 4.74 (s, 2H), 4.62 (s, 2H). LC/MS (ESI) m/z: 603 (M + H)⁺. RT (Method A): 1.63 min. |
| 218^{b} | 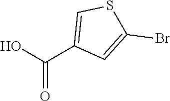 | 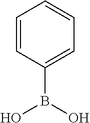 | 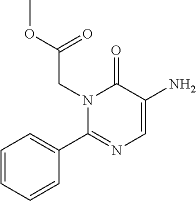 [SMILES: COC(=O)Cn1c(-c2ccccc2)ncc(N)c1=O] | 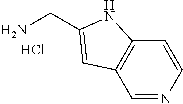 [SMILES: NCc1cc2cnccc2[nH]1 ; Cl] | ¹H NMR (400 MHz, CD₃OD) δ 9.00 (s, 1H), 8.87 (s, 1H), 8.48 (s, 1H), 8.24 (d, J = 1.4 Hz, 1H), 8.18 (d, J = 6.3 Hz, 1H), 7.88 (d, J = 1.4 Hz, 1H), 7.71 (d, J = 1.3 Hz, 1H), 7.69 (s, 1H), 7.62 (d, J = 6.2 Hz, 1H), 7.58-7.56 (m, 2H), 7.53 (d, J = 7.4 Hz, 1H), 7.47 (d, J = 7.8 Hz, 2H), 7.43 (d, J = 7.9 Hz, 2H), 7.36 (t, J = 7.4 Hz, 1H), 6.68 (s, 1H), 4.73 (s, 2H), 4.60 (s, 2H). LC/MS (ESI) (m/z): |
| | | | | | 561 (M + H)⁺. RT (Method A): |
| | | | | | 1.49 min. |
| 220 | 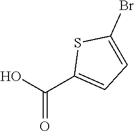 | 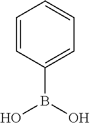 | 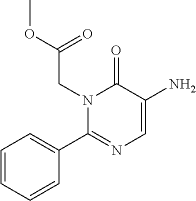 | 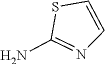 | LC/MS (ESI) (m/z): 514 (M + H)⁺. RT (Method A): 2.43 min. |
| 235 | 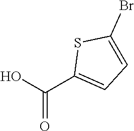 | 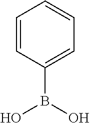 | 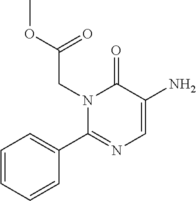 | 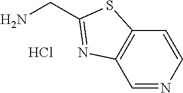 [SMILES: NCc1nc2cnccc2s1 ; Cl] | LC/MS (ESI) (m/z): 565 (M + H)⁺. RT (Method A): 2.21 min. |
| 240ª | 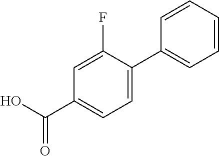 [SMILES: O=C(O)c1ccc(-c2ccccc2)c(F)c1] | | 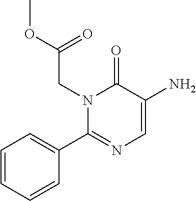 [SMILES: COC(=O)Cn1c(-c2ccccc2)ncc(N)c1=O] | 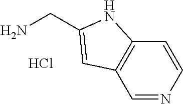 [SMILES: NCc1cc2cnccc2[nH]1 ; Cl] | ¹H NMR (400 MHz, DMSO-d₆) δ 11.35 (s, 1H), 9.78 (s, 1H), 8.83- 8.71 (m, 3H), 8.12 (d, J = 5.6 Hz, 1H), 7.93 (d, J = 9.8 Hz, 2H), 7.73 (t, J = 7.9 Hz, 1H), 7.64 (d, J = 8.2 Hz, 2H), 7.60-7.55 (m, 3H), 7.53 (d, J = 7.7 Hz, 2H), 7.47 (t, J = 7.3 Hz, 3H), 7.33 (d, J = 5.7 Hz, 1H), 6.33 (s, 1H), 4.60 (s, 2H), 4.43 (d, J = 5.5 Hz, 2H). LC/MS (ESI) |
| | | | | | m/z: 573 (M + H)⁺. |
| | | | | | RT (Method A): 1.60 min. |
| 256 | 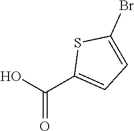 | 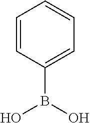 | 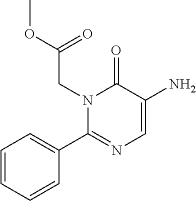 | 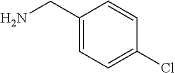 | LC/MS (ESI) (m/z): 555 (M + H)⁺. RT (Method A): 2.76 min. |

^{a}Steps 2-4 only.
^{b}Step 1 was performed in a mixture of DME and H₂O in the presence of Pd(PPh₃)₄ and NaHCO₃.
^{c}The deprotection reaction was performed with 1,5,7-triazabicyclo[4.4.0]dec-5-ene (TBD) in a mixture of MeCN and H₂O.

### Scheme 63. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-3-(thiazol-2-yl)benzamide (Compound 167)

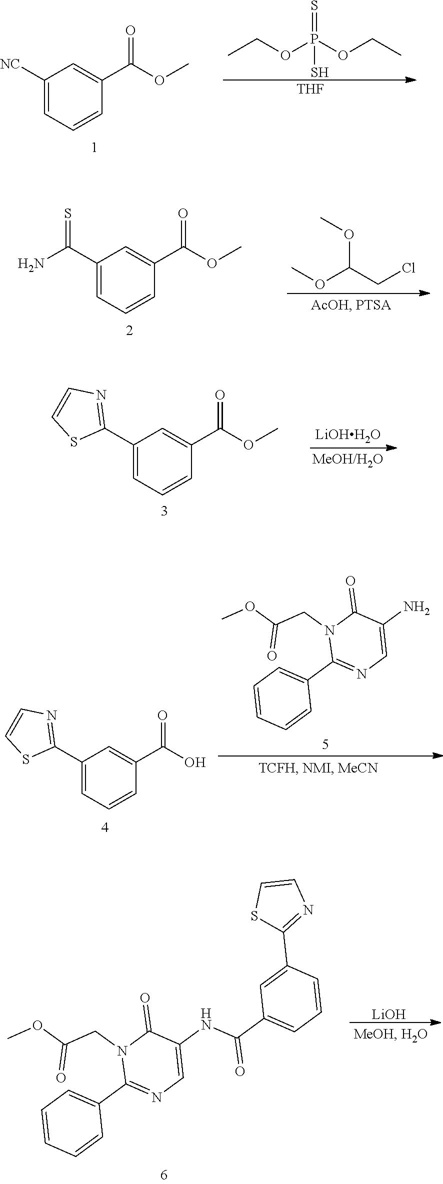

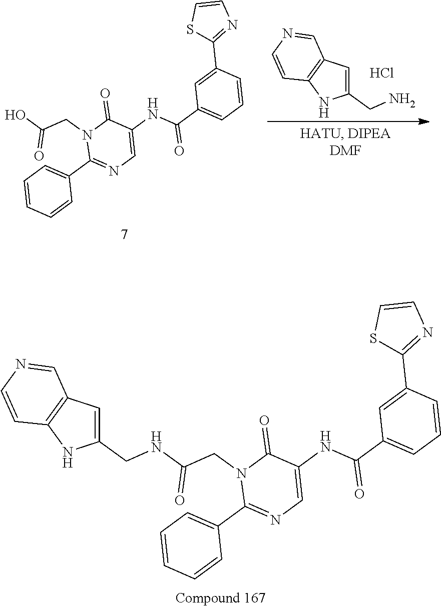

### Step 1: Methyl 3-carbamothioylbenzoate (2)

To a solution of methyl 3-cyanobenzoate (2 g, 12.4 mmol) in THF/water (20 mL, v/v=3/1) was added O,O-diethyl S-hydrogen phosphorodithioate (2.8 g, 14.9 mmol) under N₂ atmosphere and the reaction mixture was stirred at 80° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give the title compound (2.3 g, yield 95.2%) as a yellow solid. LC/MS (ESI) m/z: 196 (M+H)⁺.

### Step 2: Methyl 3-(thiazol-2-yl)benzoate (3)

To a solution of methyl 3-carbamothioylbenzoate (1.2 g, 6.1 mmol) in AcOH (12 mL) was added 2-chloro-1,1-dimethoxyethane (2.3 g, 18.3 mmol) and TsOH (50 mg, 0.29 mmol) under N₂ atmosphere and the reaction mixture was stirred at 120° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-5% EtOAc in PE) to give the title compound (1.1 g, yield 82.0%) as a yellow solid. LC/MS (ESI) m/z: 220 (M+H)⁺.

### Step 3: 3-(Thiazol-2-yl)benzoic acid (4)

To a solution of methyl 3-(thiazol-2-yl)benzoate (200 mg, 0.91 mmol) in MeOH/water (3 mL, v/v=1/1) was added LiOH (130 mg, 5.5 mmol) under N₂ atmosphere and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound 4 (170 mg, yield 91.2%) as a white solid, which was used directly in the next step without further purification. LC/MS (ESI) m/z: 206 (M+H)⁺.

### Step 4: Methyl 2-(6-oxo-2-phenyl-5-(3-(thiazol-2-yl)benzamido)pyrimidin-1(6H)-yl)acetate (5)

To a solution of 3-(thiazol-2-yl)benzoic acid (95 mg, 0.46 mmol) in MeCN (2 mL) was added methyl 2-(5-amino-6-oxo-2-phenylpyrimidin-1(6H)-yl)acetate (120 mg, 0.46 mmol), TCFH (393 mg, 1.4 mmol) and NMI (115 mg, 1.4 mmol) under N₂ atmosphere and the reaction mixture was stirred at room temperature for 10 minutes. The mixture was diluted with EtOAc and washed with water and brine. The organic layer was dried over anhydrous Na₂SO₄, filtered, and concentrated under reduced pressure to dryness. The residue was purified by flash chromatography (silica gel, 0-40% EtOAc in PE) to give the title compound (190 mg, yield 92.6%) as a white solid. LC/MS (ESI) m/z: 447 (M+H)⁺.

### Step 5: 2-(6-Oxo-2-phenyl-5-(3-(thiazol-2-yl)benzamido)pyrimidin-1(6H)-yl)acetic acid (6)

To a solution of methyl 2-(6-oxo-2-phenyl-5-(3-(thiazol-2-yl)benzamido)pyrimidin-1(6H)-yl)acetate (190 mg, 0.43 mmol) in MeOH/water (3 mL, v/v=1/1) was added LiOH (130 mg, 5.5 mmol) under N₂ atmosphere and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (170 mg, yield 91.5%) as a white solid, which was used directly in the next step without further purification. LC/MS (ESI) m/z: 433 (M+H)⁺.

### Step 6: N-(1-(2-(((1H-pyrrolo[3,2-c]pyridine-2-yl)methyl)amino)-2-oxoethyl)-6-oxo-2-phenyl-1,6-dihydropyrimidin-5-yl)-3-(thiazol-2-yl)benzamide (Compound 167)

To a solution of 2-(6-oxo-2-phenyl-5-(3-(thiazol-2-yl)benzamido)pyrimidin-1(6H)-yl)acetic acid (150 mg, 0.35 mmol) in DMF (2 mL) was added (1H-pyrrolo[3,2-c]pyridine-2-yl)methanamine hydrochloride (77 mg, 0.42 mmol), HATU (156 mg, 0.42 mmol) and DIPEA (180 mg, 1.4 mmol) under N₂ atmosphere and the reaction mixture was stirred at room temperature for 10 minutes. The mixture was diluted with EtOAc and washed with water and brine. The organic layer was dried over anhydrous Na₂SO₄, filtered, and concentrated under reduced pressure to dryness. The residue was purified by flash chromatography (silica gel, 0-8% MeOH in DCM) and further purified prep-HPLC to give Compound 167 (24 mg, yield 12.2%) as a white solid. ¹H NMR (400 MHz, DMSO-d₆) δ 12.23 (s, 1H), 9.86 (s, 1H), 9.05 (s, 1H), 8.89 (t, J=5.7 Hz, 1H), 8.79 (s, 1H), 8.51 (s, 1H), 8.29 (s, 1H), 8.20 (d, J=7.8 Hz, 1H), 8.13 (s, 1H), 8.07 (d, J=7.9 Hz, 1H), 8.00 (d, J=3.2 Hz, 1H), 7.88 (d, J=3.2 Hz, 1H), 7.70 (dd, J=16.3, 8.5 Hz, 2H), 7.59-7.57 (m, 2H), 7.54 (d, J=7.4 Hz, 1H), 7.47 (t, J=7.4 Hz, 2H), 6.63 (s, 1H), 4.62 (s, 2H), 4.51 (d, J=5.5 Hz, 2H). LC/MS (ESI) m/z: 562 (M+H)⁺.

### Scheme 64. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyl-1,2,4-oxadiazole-3-carboxamide (Compound 219)

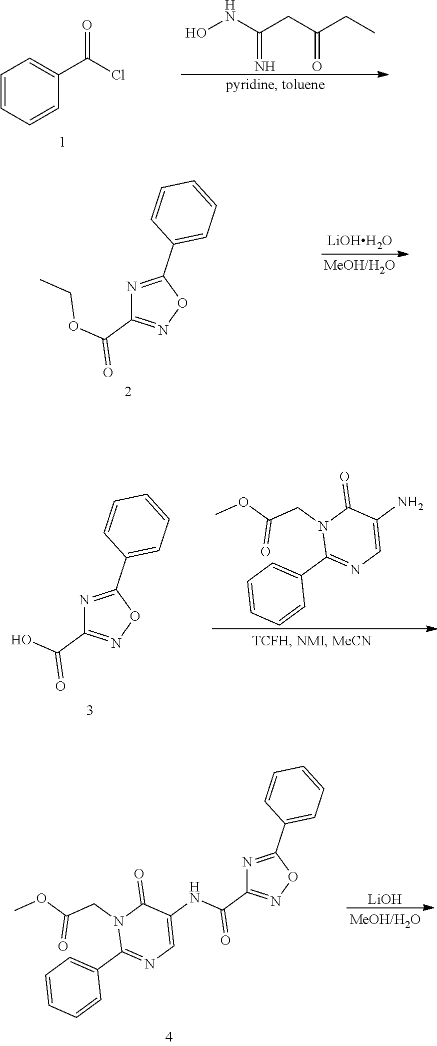

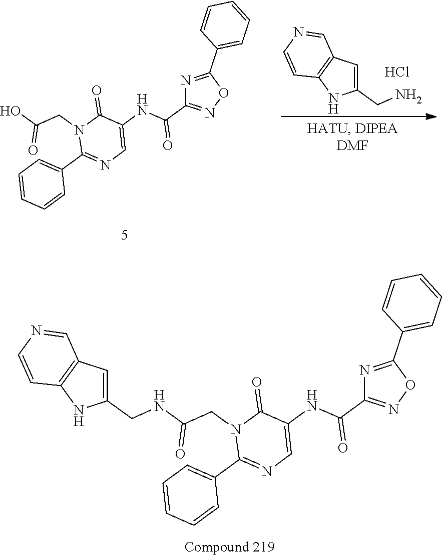 [SMILES: O=C(O)Cn1c(-c2ccccc2)ncc(NC(=O)c2noc(-c3ccccc3)n2)c1=O ; NCc1cc2cnccc2[nH]1 ; Cl ; O=C(Cn1c(-c2ccccc2)ncc(NC(=O)c2noc(-c3ccccc3)n2)c1=O)NCc1cc2cnccc2[nH]1]

### Step 1: Ethyl 5-phenyl-1,2,4-oxadiazole-3-carboxylate (2)

To a solution of benzoyl chloride (1.3 g, 9.1 mmol) in toluene (15 mL) was added N-hydroxy-3-oxopentanimidamide (1 g, 7.6 mmol) and pyridine (720 mg, 9.1 mmol) under N₂ atmosphere and the reaction mixture was stirred at 120° C. for 4 hours. The mixture was diluted with EtOAc and washed with water and brine. The organic layer was dried over anhydrous Na₂SO₄, filtered, and concentrated under reduced pressure to dryness. The residue was purified by flash chromatography (silica gel, 0-30% EtOAc in PE) to give the title compound (1.3 g, yield 78.9%) as a white solid. LC/MS (ESI) m/z: 219 (M+H)⁺.

### Step 2: 5-Phenyl-1,2,4-oxadiazole-3-carboxylic acid (3)

To a solution of ethyl 5-phenyl-1,2,4-oxadiazole-3-carboxylate (1.3 g, 6.0 mmol) in MeOH/water (15 mL, v/v=1/1) was added LiOH (864 mg, 36 mmol) and the reaction mixture was stirred at room temperature for 20 minutes. The mixture was acidified with 1 N aq. HCl to pH˜3 and extracted with EtOAc twice. The combined organic layers were dried over anhydrous Na₂SO₄, filtered, and concentrated under reduced pressure to give the title compound (1.1 g, yield 95.0%) as a white solid, which was used directly in the next step without further purification. LC/MS (ESI) m/z: 191 (M+H)⁺.

### Step 3: Methyl 2-(6-oxo-2-phenyl-5-(5-phenyl-1,2,4-oxadiazole-3-carboxamido) pyrimidin-1(6H)-yl)acetate (4)

To a solution of 5-phenyl-1,2,4-oxadiazole-3-carboxylic acid (73 mg, 0.39 mmol) in MeCN (2 mL) was added methyl 2-(5-amino-6-oxo-2-phenylpyrimidin-1(6H)-yl)acetate (100 mg, 0.39 mmol), TCFH (329 mg, 1.2 mmol) and NMI (98 mg, 1.2 mmol) under N₂ atmosphere and the reaction mixture was stirred at room temperature for 10 minutes. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (120 mg, yield 71.4%) as a white solid. LC/MS (ESI) m/z: 432 (M+H)⁺.

### Step 4: 2-(6-Oxo-2-phenyl-5-(5-phenyl-1,2,4-oxadiazole-3-carboxamido)pyrimidin-1(6H)-yl)acetic acid (5)

To a solution of methyl 2-(6-oxo-2-phenyl-5-(5-phenyl-1,2,4-oxadiazole-3-carboxamido)pyrimidin-1(6H)-yl)acetate (120 mg, 0.28 mmol) in MeOH/water (2 mL, v/v=1/1) was added LiOH (86 mg, 3.6 mmol) and the reaction mixture was stirred at room temperature for 20 minutes. The mixture was acidified with 1N aq. HCl to pH˜3 and extracted with EtOAc twice. The combined organic layers were dried over anhydrous Na₂SO₄, filtered, and concentrated under reduced pressure to give the title compound (110 mg, yield 96.4%) as a white solid, which was used directly in the next step without further purification. LC/MS (ESI) m/z: 418 (M+H)⁺.

### Step 5: N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyl-1,2,4-oxadiazole-3-carboxamide (Compound 219)

To a solution of 2-(6-oxo-2-phenyl-5-(5-phenyl-1,2,4-oxadiazole-3-carboxamido) pyrimidin-1(6H)-yl)acetic acid (80 mg, 0.19 mmol) in DMF (1.5 mL) was added (1H-pyrrolo[3,2-c]pyridine-2-yl)methanamine hydrochloride (35 mg, 0.19 mmol), HATU (87 mg, 0.23 mmol) and DIPEA (98 mg, 0.76 mmol) under N₂ atmosphere and the reaction mixture was stirred at room temperature for 10 minutes. The mixture was diluted with EtOAc, washed with water and brine, dried over anhydrous Na₂SO₄, filtered, and concentrated under reduced pressure to dryness. The residue was purified by flash chromatography (silica gel, 0-8% MeOH in DCM) and further purified by prep-HPLC to give Compound 219 (24 mg, yield 23.1%) as a white solid. ¹H NMR (400 MHz, CD₃OD) δ 9.09 (s, 1H), 8.93 (s, 1H), 8.41 (s, 1H), 8.27 (d, J=7.1 Hz, 2H), 8.21 (d, J=6.4 Hz, 1H), 7.76-7.71 (m, 2H), 7.67 (t, J=7.6 Hz, 2H), 7.61-7.58 (m, 2H), 7.55 (d, J=7.6 Hz, 1H), 7.48 (t, J=7.4 Hz, 2H), 6.75 (s, 1H), 4.76 (s, 2H), 4.63 (s, 2H). LC/MS (ESI) m/z: 547 (M+H)⁺. RT (Method A): 1.15 min.

### Scheme 65. Synthesis of 2-(5-((Dibenzo[b,d]furan-2-ylmethyl)amino)-6-oxo-2-phenylpyrimidin-1(6H)-yl)-N-(thieno[3,2-c]pyridin-2-ylmethyl)acetamide (Compound 141)

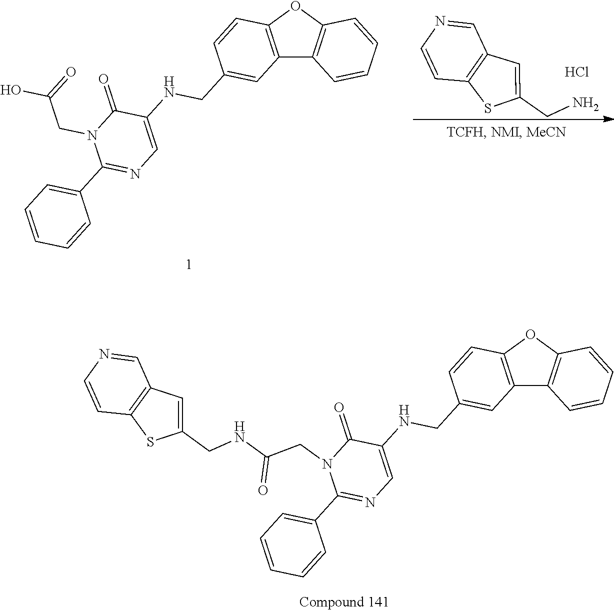

To a solution of 2-(5-((dibenzo[b,d]furan-2-ylmethyl)amino)-6-oxo-2-phenylpyrimidin-1(6H)-yl)acetic acid (43 mg, 0.10 mmol) and thieno[3,2-c]pyridin-2-ylmethanamine hydrochloride (30 mg, 0.15 mmol) in MeCN (1.0 mL) was added NMI (13 mg, 0.15 mmol) and TCFH (43 mg, 0.15 mmol) at 0° C. The mixture was stirred at 25° C. for 0.5 hour. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=15:1) and further purified by prep-HPLC to give Compound 141 (4.2 mg, yield 7.35%) as a white solid. ¹H NMR (400 MHz, DMSO-d₆) δ 9.04 (s, 1H), 8.89 (m, 1H), 8.38 (d, J=5.5 Hz, 1H), 8.17 (s, 1H), 8.12-8.10 (m, 1H), 8.01 (d, J=5.5 Hz, 1H), 7.70-7.65 (m, 2H), 7.59-7.55 (m, 1H), 7.53-7.49 (m, 1H), 7.42 (s, 1H), 7.40 (s, 2H), 7.39-7.36 (m, 3H), 7.35 (s, 1H), 7.10 (s, 1H), 6.34 (m, 1H), 4.57 (d, J=5.8 Hz, 2H), 4.50 (d, J=6.2 Hz, 2H), 4.47 (s, 2H). LC/MS (ESI) m/z: 572 (M+H)⁺. RT (Method A): 1.81 min.

### Scheme 66. Synthesis of N-((1H-pyrrolo[3,2-c]pyridine-2-yl)methyl)-2-(5-((3-(4-(3-methyloxetan-3-yl)phenyl)propyl)amino)-6-oxo-2-phenylpyrimidin-1(6H)-yl)acetamide (Compound 180)

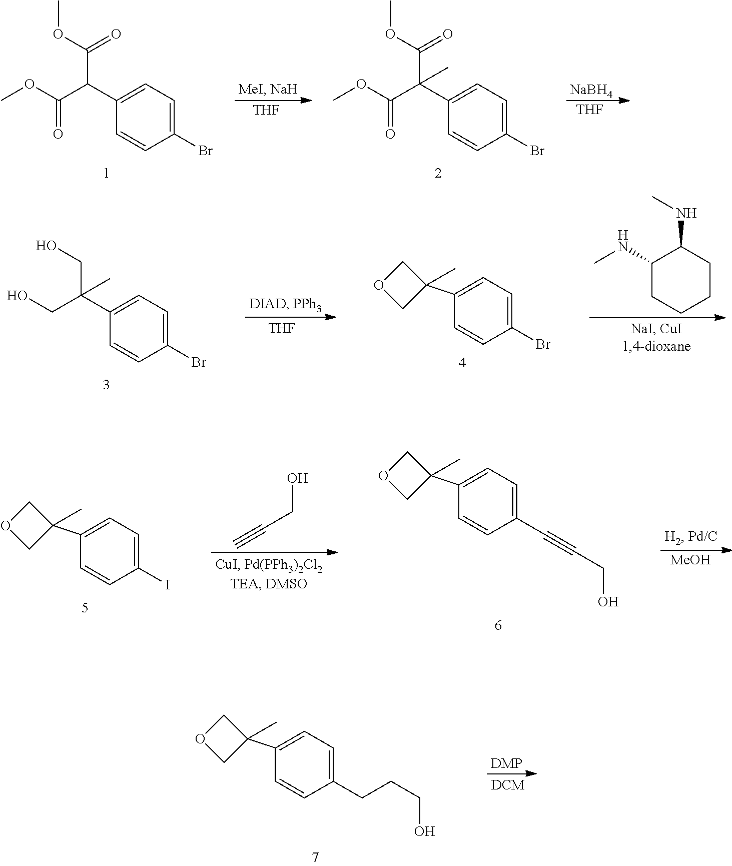

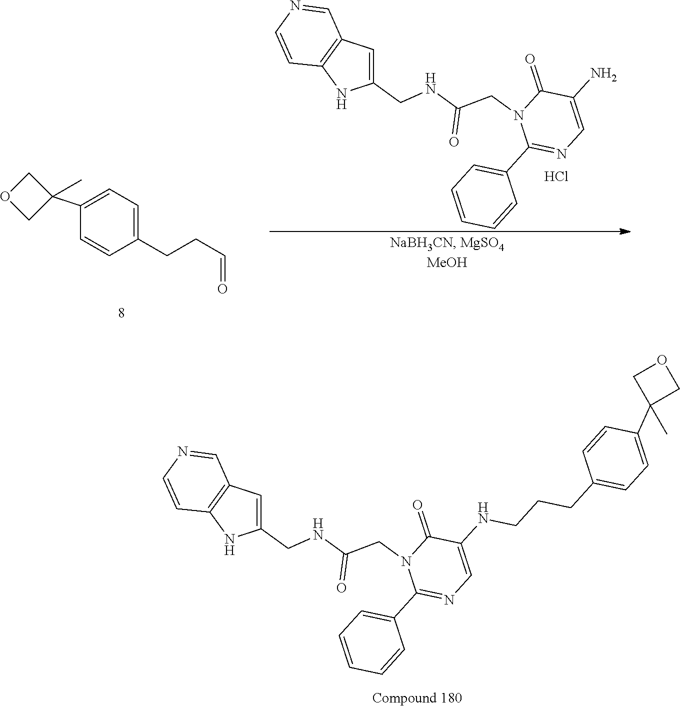 [SMILES: CC1(c2ccc(CCC=O)cc2)COC1 ; Nc1cnc(-c2ccccc2)n(CC(=O)NCc2cc3cnccc3[nH]2)c1=O ; Cl ; CC1(c2ccc(CCCNc3cnc(-c4ccccc4)n(CC(=O)NCc4cc5cnccc5[nH]4)c3=O)cc2)COC1]

### Step 1: Dimethyl 2-(4-bromophenyl)-2-methylmalonate (2)

To a solution of dimethyl 2-(4-bromophenyl)malonate (1.0 g, 3.48 mmol) in THF (10 mL) was added NaH (209 mg, 60% dispersion in mineral oil) at 0° C. under N₂ atmosphere and the reaction mixture was stirred under N₂ atmosphere at 0° C. for 30 minutes. Subsequently, to the above mixture was added a solution of CH₃I (984 mg, 6.96 mmol) drop-wisely at 0° C. under N₂ atmosphere and the resulting mixture was stirred at room temperature under N₂ atmosphere for 2 hours. The mixture was quenched with saturated aq.NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (832 mg, yield 79.7%) as a white solid. ¹H NMR (400 MHz, CDCl₃) δ 7.49 (s, 1H), 7.46 (s, 1H), 7.25 (s, 1H), 7.23 (s, 1H), 3.76 (s, 6H), 1.85 (s, 3H).

### Step 2: 2-(4-Bromophenyl)-2-methylpropane-1,3-diol (3)

To a mixture of dimethyl 2-(4-bromophenyl)-2-methylmalonate (832 mg, 2.76 mmol) in THF (8 mL) was added NaBH₄ (211 mg, 5.58 mmol) under N₂ atmosphere and the reaction mixture was stirred at room temperature for 8 hours. The mixture was quenched with saturated aq.NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (522 mg, yield 77.2%) as a white solid. LC/MS (ESI) m/z: 245 (M+H)⁺.

### Step 3: 3-(4-Bromophenyl)-3-methyloxetane (4)

To a solution of 2-(4-bromophenyl)-2-methylpropane-1,3-diol (522 mg, 2.13 mmol) in toluene (5 mL) was added PPh₃ (1.12 g, 4.28 mmol) and DIAD (865 mg, 4.28 mmol) at 0° C. under N₂ atmosphere and the mixture was stirred in CEM-microwave reactor at 140° C. for 20 minutes. The reaction mixture was diluted with EtOAc, washed with water and brine, dried over anhydrous Na₂SO₄, filtered, and concentrated under reduced pressure to dryness. The residue was purified by flash chromatography (silica gel, 0-40% EtOAc in PE) to give the title compound (210 mg, yield 43.4%) as a colorless oil. ¹H NMR (400 MHz, CDCl₃) δ 7.48 (d, J=8.5 Hz, 2H), 7.09 (d, J=8.5 Hz, 2H), 4.91 (d, J=5.6 Hz, 2H), 4.63 (d, J=5.6 Hz, 2H), 1.71 (s, 3H).

### Step 4: 3-(4-Iodophenyl)-3-methyloxetane (5)

To a mixture of 3-(4-bromophenyl)-3-methyloxetane (263 mg, 1.16 mmol) and NaI (1.1 g, 6.98 mmol) in 1,4-dioxnae (6 mL) was added CuI (22 mg, 0.11 mmol) and (1S,2S)—N1,N2-dimethylcyclohexane-1,2-diamine (496 mg, 3.49 mmol) under N₂ atmosphere, the mixture was degassed under N₂ atmosphere for three times and stirred at 120° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-40% EtOAc in PE) to give the title compound (268 mg, yield 84.2%) as a white solid.

### Step 5: 3-(4-(3-Methyloxetan-3-yl)phenyl)prop-2-yn-1-ol (6)

To a mixture of 3-(4-iodophenyl)-3-methyloxetane (268 mg, 0.97 mmol) and prop-2-yn-1-ol (96 mg, 1.78 mmol) in TEA (2 mL) and DMSO (2 mL) was added CuI (23 mg, 0.12 mmol), Pd(PPh₃)₂Cl₂ (84 mg, 0.12 mmol) under N₂ atmosphere, the mixture was degassed under N₂ atmosphere for three times and stirred at 50° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give the title compound (196 mg, yield 99.9%) as a yellow solid. ¹H NMR (400 MHz, CDCl₃) δ 7.44 (s, 1H), 7.42 (s, 1H), 7.17 (s, 1H), 7.15 (s, 1H), 4.94 (d, J=5.6 Hz, 2H), 4.63 (d, J=5.7 Hz, 2H), 4.50 (d, J=5.7 Hz, 2H), 1.72 (s, 3H). LC/MS (ESI) m/z: 203 (M+H)⁺.

### Step 6: 3-(4-(3-Methyloxetan-3-yl)phenyl)propan-1-ol (7)

To a solution of 3-(4-(3-methyloxetan-3-yl)phenyl)prop-2-yn-1-ol (196 mg, 0.97 mmol) in MeOH (3 mL) was added Pd/C (40 mg, 10% wt.), the mixture was degassed under N₂ atmosphere for three times and stirred under a H₂ balloon at room temperature overnight. The mixture was filtered, and the filtrate was concentrated to dryness to give the title compound (200 mg, yield 99.9%) as a yellow oil, which was used directly in the next step without further purification. ¹H NMR (400 MHz, CDCl₃) δ 7.20 (d, J=8.1 Hz, 2H), 7.13 (d, J=8.2 Hz, 2H), 4.96 (d, J=5.5 Hz, 2H), 4.62 (d, J=5.5 Hz, 2H), 3.69-3.65 (m, 2H), 2.75-2.66 (m, 2H), 1.94-1.85 (m, 2H), 1.72 (s, 3H).

### Step 7: 3-(4-(3-Methyloxetan-3-yl)phenyl)propanal (8)

To a solution of 3-(4-(3-methyloxetan-3-yl)phenyl)propan-1-ol (50 mg, 0.24 mmol) in DCM (2 mL) was added Dess-Martin periodinane (205 mg, 0.48 mmol) under N₂ atmosphere and the reaction mixture was stirred at room temperature for 20 minutes. The mixture was diluted with DCM, washed with saturated aq.NaHCO₃ solution and brine, dried over anhydrous Na₂SO₄, filtered, and concentrated under reduced pressure to give the title compound (45 mg, yield 91.9%) as a yellow oil, which was used directly in the next step without further purification. ¹H NMR (400 MHz, CDCl₃) δ 9.76 (s, 1H), 7.50 (d, J=8.2 Hz, 2H), 7.23-7.20 (m, 2H), 4.89 (m, 2H), 4.61 (m, 2H), 2.89 (m, 2H), 2.72 (m, 2H), 1.31 (s, 3H).

### Step 8: N-((1H-pyrrolo[3,2-c]pyridine-2-yl)methyl)-2-(5-((3-(4-(3-methyloxetan-3-yl)phenyl)propyl)amino)-6-oxo-2-phenylpyrimidin-1(6H)-yl)acetamide (Compound 180)

To a mixture of 3-(4-(3-methyloxetan-3-yl)phenyl)propanal (45 mg, 0.22 mmol) and N-((1H-pyrrolo[3,2-c]pyridine-2-yl)methyl)-2-(5-amino-6-oxo-2-phenylpyrimidin-1(6H)-yl)acetamide hydrochloride (30 mg, 0.08 mol) in MeOH (3 mL) was added MgSO₄ (30 mg, 0.24 mmol) and NaBH₃CN (44 mg, 0.66 mmol) under N₂ atmosphere and the reaction mixture was stirred at 50° C. for 2 hours. The mixture was quenched with saturated aq.NH₄Cl solution and extracted with EtOAc twice. The combined organic layers were washed with brine, dried over anhydrous Na₂SO₄, filtered, and concentrated under reduced pressure to dryness. The residue was purified by prep-HPLC to give Compound 180 (5 mg, yield 4.03%) as a white solid. ¹H NMR (400 MHz, CD₃OD) δ 8.70 (s, 1H), 8.10 (d, J=5.8 Hz, 1H), 7.48-7.44 (m, 3H), 7.40-7.37 (m, 3H), 7.23 (d, J=8.2 Hz, 2H), 7.14 (d, J=8.2 Hz, 2H), 7.08 (s, 1H), 6.47 (s, 1H), 4.95 (d, J=5.6 Hz, 2H), 4.63 (d, J=5.6 Hz, 4H), 4.54 (s, 2H), 3.18 (t, J=6.9 Hz, 2H), 2.74 (t, J=7.6 Hz, 2H), 2.02-1.96 (m, 2H), 1.68 (s, 3H). LC/MS (ESI) m/z: 563 (M+H)⁺. RT (Method A): 1.37 min.

### Scheme 67. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-3-fluoro-[1,1′-biphenyl]-4-carboxamide (Compound 166)

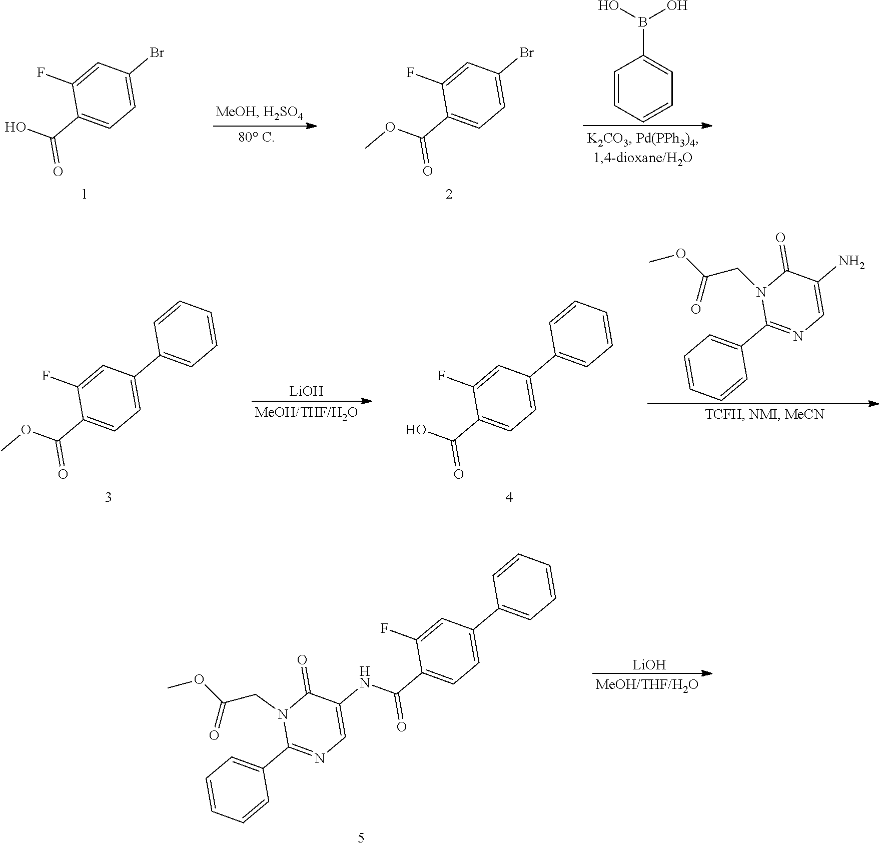

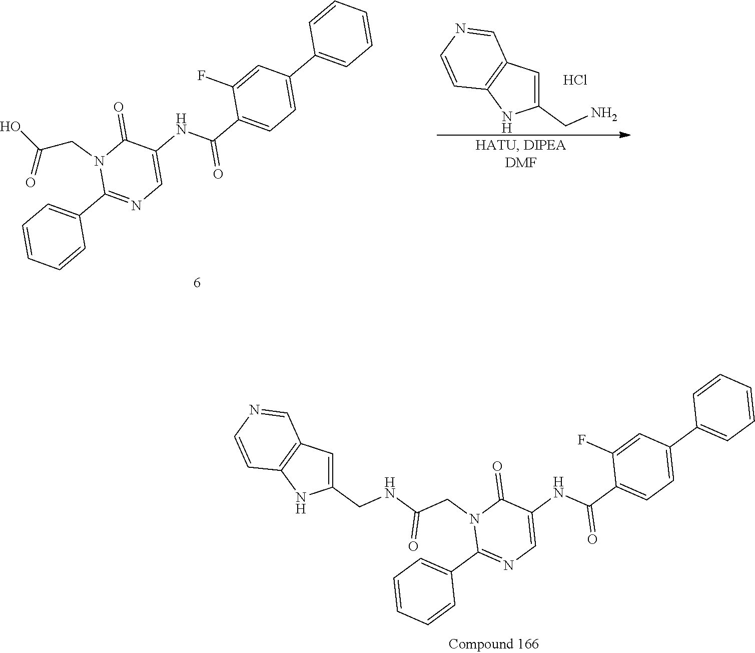 [SMILES: O=C(O)Cn1c(-c2ccccc2)ncc(NC(=O)c2ccc(-c3ccccc3)cc2F)c1=O ; NCc1cc2cnccc2[nH]1 ; Cl ; O=C(Cn1c(-c2ccccc2)ncc(NC(=O)c2ccc(-c3ccccc3)cc2F)c1=O)NCc1cc2cnccc2[nH]1]

### Step 1: Methyl 4-bromo-2-fluorobenzoate (2)

To a solution of 4-bromo-2-fluorobenzoic acid (1.50 g, 6.88 mmol) in MeOH (10 mL) was added conc.H₂SO₄ (0.5 ml) and the reaction mixture was stirred at 80° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-25% EtOAc in PE) to give the title compound (1.30 g, yield 81.4%) as a white solid.

### Step 2: Methyl 3-fluoro-[1,1′-biphenyl]-4-carboxylate (3)

To a mixture of methyl 4-bromo-2-fluorobenzoate (400 mg, 1.72 mmol) and phenylboronic acid (230 mg, 1.89 mmol) in 1,4-dioxane (8 mL) and water (3 mL) was added K₂CO₃ (711 mg, 5.15 mmol) and Pd(PPh₃)₄ (198 mg, 0.17 mmol) at 25° C. under N₂ atmosphere, the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (352 mg, yield 89.1%) as a yellow oil. LC/MS (ESI) m/z: 231 (M+H)⁺.

### Step 3: 3-Fluoro-[1,1′-biphenyl]-4-carboxylic acid (4)

To a solution of methyl 3-fluoro-[1,1′-biphenyl]-4-carboxylate (352 mg, 1.41 mmol) in MeOH/THF/water (3 mL, 2/1/1) was added LiOH·H₂O (77 mg, 1.84 mmol) under N₂ atmosphere and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (325 mg, yield 98.3%) as a yellow solid, which was used directly in the next step without further purification. LC/MS (ESI) m/z: 217 (M+H)⁺.

### Step 4: Methyl 2-(5-(3-fluoro-[1,1′-biphenyl]-4-carboxamido)-6-oxo-2-phenylpyrimidin-1(6H)-yl)acetate (5)

To a mixture of 3-fluoro-[1,1′-biphenyl]-4-carboxylic acid (150 mg, 1.04 mmol) and methyl 2-(5-amino-6-oxo-2-phenylpyrimidin-1(6H)-yl)acetate (120 mg, 0.46 mmol) in MeCN (2 mL) was added TCFH (389 mg, 1.39 mmol) and NMI (114 mg, 1.39 mmol) under N₂ atmosphere and the reaction mixture was stirred at room temperature for 2 hours. The mixture was diluted with EtOAc, washed with saturated aq.NaHCO₃ solution and brine, dried over anhydrous Na₂SO₄, filtered, and concentrated under reduced pressure to dryness. The residue was purified by flash chromatography (silica gel, 0-50% EtOAc in PE) to give the title compound (190 mg, yield 89.7%) as a white solid. LC/MS (ESI) m/z: 458 (M+H)⁺.

### Step 5: 2-(5-(3-Fluoro-[1,1′-biphenyl]-4-carboxamido)-6-oxo-2-phenylpyrimidin-1(6H)-yl)acetic acid (6)

To a solution of methyl 2-(5-(3-fluoro-[1,1′-biphenyl]-4-carboxamido)-6-oxo-2-phenylpyrimidin-1(6H)-yl)acetate (190 mg, 0.42 mmol) in MeOH/THF/water (3 mL, 2/1/1) was added LiOH·H₂O (20 mg, 0.54 mmol) and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (125 mg, yield 99.4%) as a yellow solid, which was used directly in the next step without further purification. LC/MS (ESI) m/z: 444 (M+H)⁺.

### Step 6: N-(1-(2-(((1H-pyrrolo[3,2-c]pyridine-2-yl)methyl)amino)-2-oxoethyl)-6-oxo-2-phenyl-1,6-dihydropyrimidin-5-yl)-3-fluoro-[1,1′-biphenyl]-4-carboxamide (Compound 166)

To a mixture of 2-(5-(3-fluoro-[1,1′-biphenyl]-4-carboxamido)-6-oxo-2-phenylpyrimidin-1(6H)-yl)acetic acid (159 mg, 0.39 mmol) and (1H-pyrrolo[3,2-c]pyridine-2-yl)methanamine hydrochloride (95 mg, 0.64 mmol) in DMF (2 mL) was added DIPEA (186 mg, 1.43 mmol) and HATU (177 mg, 0.47 mmol) under N₂ atmosphere and the reaction mixture was stirred at room temperature for 2 hours. The mixture was diluted with EtOAc, washed with saturated aq.NaHCO₃ solution and brine, dried over anhydrous Na₂SO₄, filtered, and concentrated under reduced pressure to dryness. The residue was purified by flash chromatography (silica gel, 0-70% EtOAc in PE) to give Compound 166 (34.9 mg, yield 16.9%) as a white solid. ¹H NMR (400 MHz, DMSO-d₆) δ 12.57 (s, 1H), 9.63 (d, J=10.4 Hz, 1H), 9.18 (s, 1H), 9.02 (s, 1H), 8.93 (t, J=5.7 Hz, 1H), 8.38 (d, J=6.7 Hz, 1H), 8.03 (t, J=8.2 Hz, 1H), 7.89 (d, J=6.6 Hz, 1H), 7.83 (s, 1H), 7.81 (s, 1H), 7.80-7.73 (m, 2H), 7.58-7.52 (m, 5H), 7.49-7.45 (m, 3H), 6.76 (s, 1H), 4.63 (s, 2H), 4.54 (d, J=5.5 Hz, 2H). LC/MS (ESI) m/z: 573 (M+H)⁺. RT (Method A): 1.66 min.

Compound 164 was prepared based on Steps 2-5 in Scheme 67:

| # | Reactant A | Reactant B | Reactant C | Reactant D |
|---|---|---|---|---|
| 164^{a} | 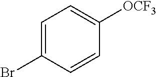 [SMILES: FC(F)(F)Oc1ccc(Br)cc1] | 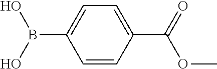 | 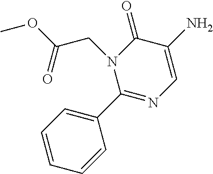 [SMILES: COC(=O)Cn1c(-c2ccccc2)ncc(N)c1=O] | 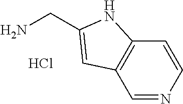 [SMILES: NCc1cc2cnccc2[nH]1 ; Cl] |
| | | | # | Characterization Data |
| | | | 164^{a} | ¹H NMR (400 MHz, DMSO-d₆) δ |
| | | | | 9.61 (s, 1H), 8.94 (d, J = 16.4 Hz, |
| | | | | 1H), 8.88-8.81 (m, 2H), 8.23 (s, |
| | | | | 1H), 8.09 (d, J = 8.2 Hz, 2H), 7.92- |
| | | | | 7.87 (m, 4H), 7.59-7.45 (m, |
| | | | | 8H), 6.51 (d, J = 13.3 Hz, 1H), |
| | | | | 4.61 (s, 2H), 4.48 (s, 2H). LC/MS |
| | | | | (ESI) m/z: 638 (M + H)⁺. RT |
| | | | | (Method A): 1.87 min. |

^{a}Step 4 was performed with TBD in a mixture of MeCN and H₂O.

### Scheme 68. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-4-(thiazol-2-yl)benzamide (Compound 172)

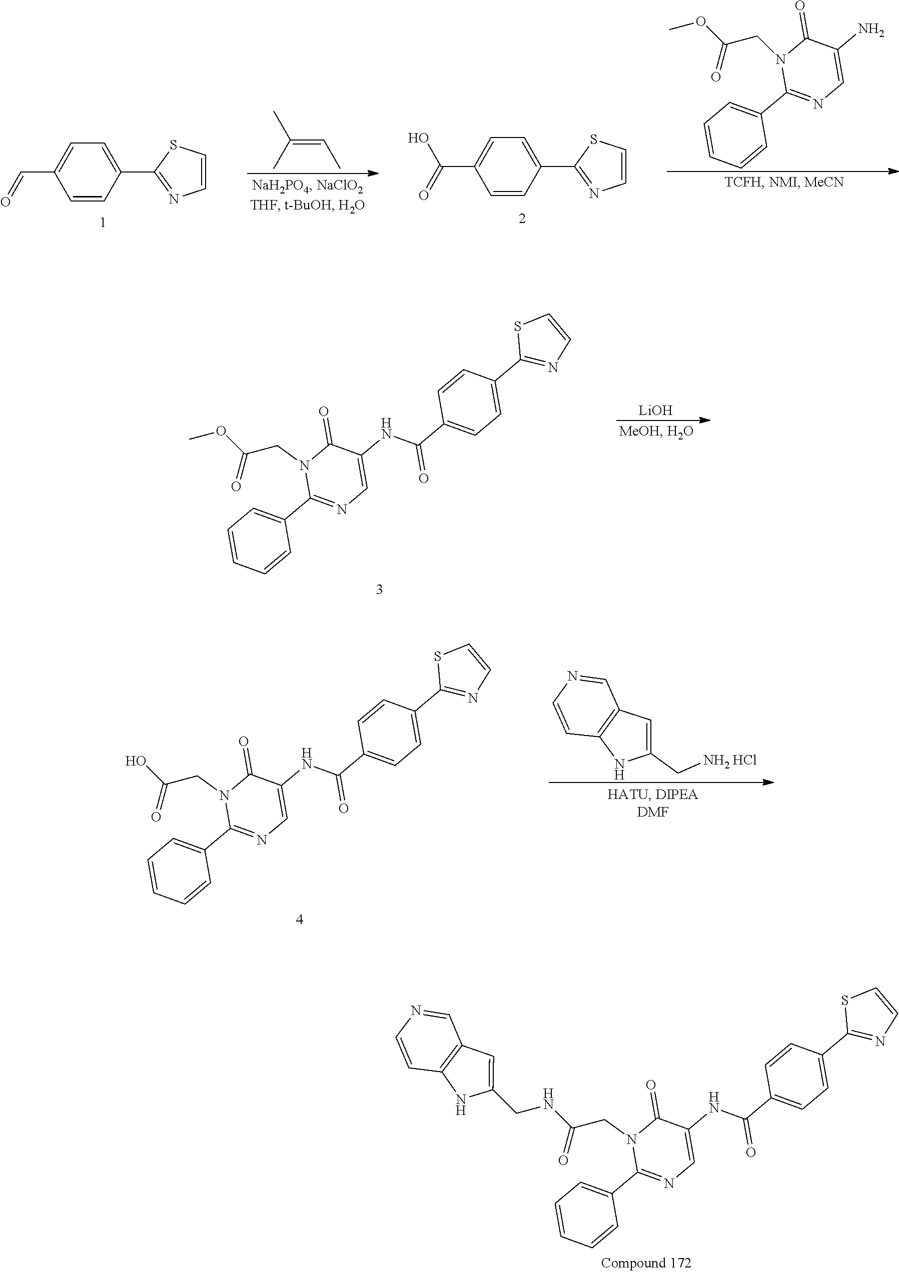

### Step 1: 4-(Thiazol-2-yl)benzoic acid (2)

To a mixture of 4-(thiazol-2-yl)benzaldehyde (500 mg, 2.64 mmol) and 2-methylbut-2-ene (1.9 g, 15.84 mmol) in THF/t-BuOH (10 mL, v/v=1/1) was added a mixture of NaH₂PO₄ (1.43 g, 15.84 mmol) and NaClO₂ (741 mg, 10.57 mmol) in water (4 mL) and the mixture was stirred at room temperature for 0.5 hour. The mixture was diluted with EtOAc, washed with water and brine, dried over anhydrous Na₂SO₄, filtered, and concentrated under reduced pressure to give the title compound (450 mg, yield 83.0%) as a white solid. LC/MS (ESI) m/z: 205 (M+H)⁺.

### Step 2: Methyl 2-(6-oxo-2-phenyl-5-(4-(thiazol-2-yl)benzamido)pyrimidin-1(6H)-yl)acetate (3)

To a mixture of 4-(thiazol-2-yl)benzoic acid (145 mg, 0.70 mmol) and methyl 2-(5-amino-6-oxo-2-phenylpyrimidin-1(6H)-yl)acetate (120 mg, 0.46 mmol) in MeCN (4 mL) was added NMI (114 mg, 1.38 mmol) and TCFH (389 mg, 1.38 mmol) and the mixture was stirred at room temperature for 1 hour. The mixture was diluted with EtOAc, washed with saturated aq.NaHCO₃ solution, dried over anhydrous Na₂SO₄, filtered, and concentrated under reduced pressure to dryness. The residue was purified by flash chromatography (silica gel, 0-10% MeOH in DCM) to give the title compound (200 mg, yield 96.8%) as a yellow solid. LC/MS (ESI) m/z: 447 (M+H)⁺.

### Step 3: 2-(6-Oxo-2-phenyl-5-(4-(thiazol-2-yl)benzamido)pyrimidin-1(6H)-yl)acetic acid (4)

To a solution of methyl 2-(6-oxo-2-phenyl-5-(4-(thiazol-2-yl)benzamido)pyrimidin-1(6H)-yl)acetate (200 mg, 0.45 mmol) in MeOH/water (4.0 mL, v/v=4/1) was added LiOH·H₂O (38 mg, 0.90 mmol) and the reaction mixture was stirred at 50° C. for 3 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (130 mg, yield 67.1%) as a yellow solid, which was used directly in the next step without further purification. LC/MS (ESI) m/z: 433 (M+H)⁺.

### Step 4: N-(1-(2-(((1H-pyrrolo[3,2-c]pyridine-2-yl)methyl)amino)-2-oxoethyl)-6-oxo-2-phenyl-1,6-dihydropyrimidin-5-yl)-4-(thiazol-2-yl)benzamide (Compound 172)

To a mixture of 2-(6-oxo-2-phenyl-5-(4-(thiazol-2-yl)benzamido)pyrimidin-1(6H)-yl)acetic acid (130 mg, 0.30 mmol) and (1H-pyrrolo[3,2-c]pyridine-2-yl)methanamine hydrochloride (88 mg, 0.60 mmol) in DMF (2 mL) was added DIPEA (193 mg, 1.49 mmol) and HATU (137 mg, 0.36 mmol) and the mixture was stirred at room temperature for 1 hour. The mixture was diluted with EtOAc, washed with saturated aq.NaHCO₃ solution, dried over anhydrous Na₂SO₄, filtered, and concentrated under reduced pressure to dryness. The residue was purified by flash chromatography (silica gel, 0-20% MeOH in DCM) and further purified by prep-HPLC to give Compound 172 (23.7 mg, yield 14.0%) as a white solid. ¹H NMR (400 MHz, DMSO-d₆) δ 11.37 (s, 1H), 9.70 (s, 1H), 8.82 (s, 1H), 8.79 (d, J=5.6 Hz, 1H), 8.75 (s, 1H), 8.15-8.10 (m, 5H), 8.02 (d, J=3.2 Hz, 1H), 7.91 (d, J=3.2 Hz, 1H), 7.58 (d, J=7.1 Hz, 2H), 7.54 (d, J=7.4 Hz, 1H), 7.50-7.45 (m, 2H), 7.33 (d, J=5.6 Hz, 1H), 6.33 (s, 1H), 4.60 (s, 2H), 4.44 (d, J=5.5 Hz, 2H). LC/MS (ESI) m/z: 562 (M+H)⁺. RT (Method A): 0.99 min.

### Scheme 69. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyl-1H-1,2,4-triazole-3-carboxamide (Compound 221)

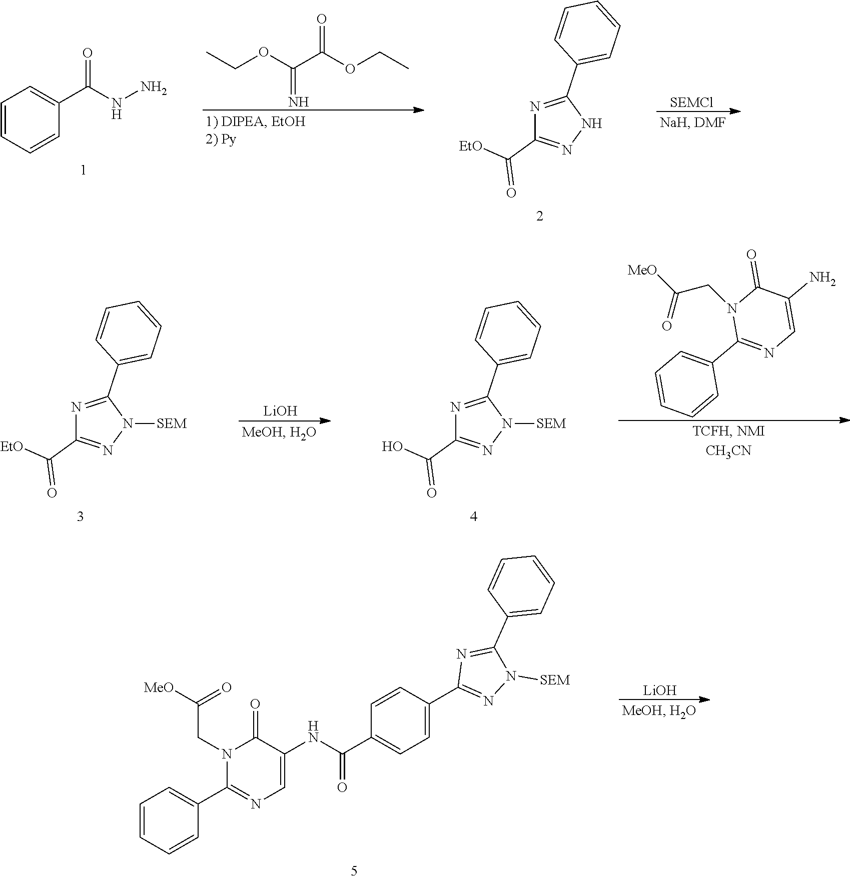

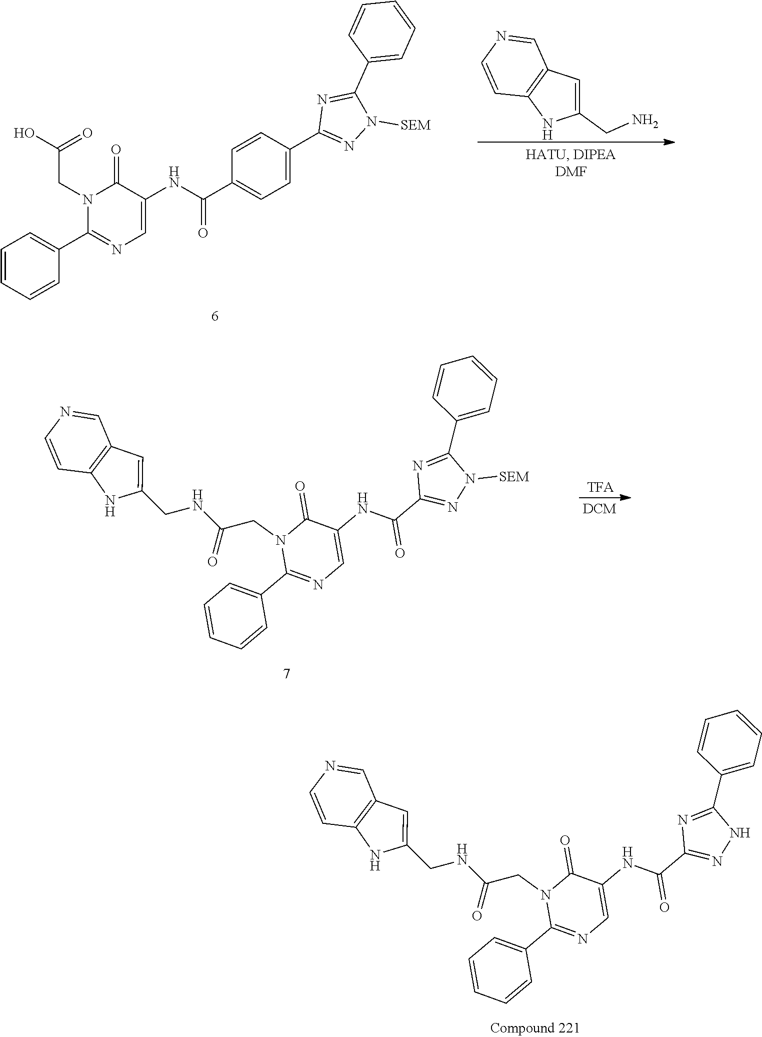 [SMILES: C[Si](C)(C)CCOCn1nc(-c2ccc(C(=O)Nc3cnc(-c4ccccc4)n(CC(=O)O)c3=O)cc2)nc1-c1ccccc1 ; NCc1cc2cnccc2[nH]1 ; C[Si](C)(C)CCOCn1nc(C(=O)Nc2cnc(-c3ccccc3)n(CC(=O)NCc3cc4cnccc4[nH]3)c2=O)nc1-c1ccccc1 ; O=C(Cn1c(-c2ccccc2)ncc(NC(=O)c2n[nH]c(-c3ccccc3)n2)c1=O)NCc1cc2cnccc2[nH]1]

### Step 1: Ethyl 5-phenyl-1H-1,2,4-triazole-3-carboxylate (2)

To a mixture of benzohydrazide (4.0 g, 29.41 mmol) and ethyl 2-ethoxy-2-iminoacetate (4.26 g, 29.41 mmol) in EtOH (70 mL) was added DIPEA (7.6 g, 58.82 mmol) at 0° C. under N₂ atmosphere and the reaction mixture was stirred at room temperature overnight. The mixture was concentrated under reduced pressure to dryness. The residue was dissolved in pyridine (50 mL) was stirred under N₂ atmosphere at 120° C. overnight. The mixture was concentrated under reduced pressure to dryness. The residue was diluted with water and extracted with DCM twice. The combined organic layers were washed with brine, dried over anhydrous Na₂SO₄, filtered, and concentrated under reduced pressure to dryness. The residue was purified by flash chromatography (silica gel, 0-4% MeOH in DCM) to give the title compound (2.8 g, yield 43.9%) as a yellow solid. LC/MS (ESI) m/z: 218 (M+H)⁺.

### Step 2: Ethyl 5-phenyl-1-((2-(trimethylsilyl)ethoxy)methyl)-1H-1,2,4-triazole-3-carboxylate (3)

To a solution of NaH (103 mg, 2.58 mmol, 60%, dispersion in mineral oil) in DMF (3 mL) was added a solution of ethyl 5-phenyl-1H-1,2,4-triazole-3-carboxylate (500 mg, 2.30 mmol) in DMF (3 mL) at 0° C. and the reaction mixture was stirred at room temperature for 10 minutes. At 0° C., SEMCl (390 mg. 2.34 mmol) was added to the stirring reaction mixture and the resulting mixture was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give the title compound (350 mg, yield 43.8%) as a yellow oil. ¹H NMR (400 MHz, DMSO-d₆) δ 8.08-8.02 (m, 2H), 7.54-7.48 (m, 3H), 5.85 (s, 2H), 4.43 (q, J=7.1 Hz, 2H), 3.68 (t, J=7.9 Hz, 2H), 1.37 (t, J=7.1 Hz, 3H), 0.85 (t, J=7.9 Hz, 2H), −0.08 (d, J=3.3 Hz, 9H).

### Step 3: 5-Phenyl-1-((2-(trimethylsilyl)ethoxy)methyl)-1H-1,2,4-triazole-3-carboxylic acid (4)

To a solution of ethyl 5-phenyl-1-((2-(trimethylsilyl)ethoxy)methyl)-1H-1,2,4-triazole-3-carboxylate (250 mg, 0.72 mmol) in MeOH (4 mL) and water (1 mL) was added LiOH (26 mg, 1.08 mmol) and the mixture was stirred at room temperature for 2 hours. The mixture was concentrated under reduced pressure to dryness to give the title compound (229 mg, yield 99.7%) as a white solid, which was used directly in the next step without further purification. LC/MS (ESI) m/z: 318 (M−H)⁻.

### Step 4: Methyl 2-(6-oxo-2-phenyl-5-(5-phenyl-1-((2-(trimethylsilyl)ethoxy)methyl)-1H-1,2,4-triazole-3-carboxamido)pyrimidin-1(6H)-yl)acetate (5)

To a mixture of 5-phenyl-1-((2-(trimethylsilyl)ethoxy)methyl)-1H-1,2,4-triazole-3-carboxylic acid (229 mg, 0.72 mmol) and methyl 2-(5-amino-6-oxo-2-phenylpyrimidin-1(6H)-yl)acetate (130 mg, 0.50 mmol) in MeCN (5 mL) was added TCFH (421 mg, 1.50 mmol) and NMI (123 mg, 1.50 mmol) under N₂ atmosphere and the reaction mixture was stirred at room temperature for 1 hour. The mixture was diluted with DCM, washed with 10% aq.NaHCO₃ solution and brine, dried over anhydrous Na₂SO₄, filtered, and concentrated under reduced pressure to dryness. The residue was purified by flash chromatography (silica gel, 0-10% EtOAc in PE) to give the title compound (210 mg, yield 74.7%) as a light oil. LC/MS (ESI) m/z: 561 (M+H)⁺.

### Step 5: 2-(6-Oxo-2-phenyl-5-(5-phenyl-1-((2-(trimethylsilyl)ethoxy)methyl)-1H-1,2,4-triazole-3-carboxamido)pyrimidin-1(6H)-yl)acetic acid (6)

To a solution of methyl 2-(6-oxo-2-phenyl-5-(5-phenyl-1-((2-(trimethylsilyl) ethoxy)methyl)-1H-1,2,4-triazole-3-carboxamido)pyrimidin-1(6H)-yl)acetate (210 mg, 0.37 mmol) in MeOH (4 mL) and water (2 mL) was added LiOH (20 mg, 0.83 mmol) and the mixture was stirred at room temperature for 1 hour. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (190 mg, yield 92.8%) as a white solid, which was used directly in the next step without further purification. LC/MS (ESI) m/z: 547 (M+H)⁺.

### Step 6: N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyl-1-((2-(trimethylsilyl)ethoxy)methyl)-1H-1,2,4-triazole-3-carboxamide (7)

To a mixture of 2-(6-oxo-2-phenyl-5-(5-phenyl-1-((2-(trimethylsilyl)ethoxy)methyl)-1H-1,2,4-triazole-3-carboxamido)pyrimidin-1(6H)-yl)acetic acid (190 mg, 0.35 mmol) and (1H-pyrrolo[3,2-c]pyridine-2-yl)methanamine hydrochloride (99 mg, 0.54 mmol) in DMF (4 mL) was added DIPEA (180 mg, 1.40 mmol) and HATU (170 mg, 0.45 mmol) under N₂ atmosphere and the reaction mixture was stirred at room temperature for 0.5 hour. The mixture was diluted with EtOAc, washed with saturated aq.NH₄Cl solution and brine, dried over anhydrous Na₂SO₄, filtered, and concentrated under reduced pressure to dryness. The residue was purified by flash chromatography (silica gel, 0-10% MeOH in DCM) to give the title compound (220 mg, yield 93.7%) as a yellow solid. LC/MS (ESI) m/z: 676 (M+H)⁺.

### Step 7: N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyl-1H-1,2,4-triazole-3-carboxamide (Compound 221)

To a solution of N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyl-1-((2-(trimethylsilyl)ethoxy)methyl)-1H-1,2,4-triazole-3-carboxamide (200 mg, 0.30 mmol) in DCM (4 mL) was added TFA (2 mL) and the reaction mixture was stirred at room temperature for 2 hours. The mixture was concentrated under reduced pressure to dryness. The residue was purified by prep-HPLC to give Compound 221 (40 mg, yield 24.8%) as a white solid. ¹H NMR (400 MHz, DMSO-d₆) δ 11.54 (s, 1H), 9.71 (s, 1H), 8.99 (s, 1H), 8.86-8.80 (m, 2H), 8.15 (d, J=7.1 Hz, 2H), 8.11-8.07 (m, 2H), 7.61-7.54 (m, 6H), 7.47 (t, J=7.3 Hz, 2H), 7.42 (d, J=5.8 Hz, 1H), 6.42 (s, 1H), 4.62 (s, 2H), 4.46 (d, J=5.5 Hz, 2H). LC/MS (ESI) m/z: 546 (M+H)⁺. RT (Method A): 0.98 min.

### Scheme 70. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-3-(oxazol-2-yl)benzamide (Compound 222)

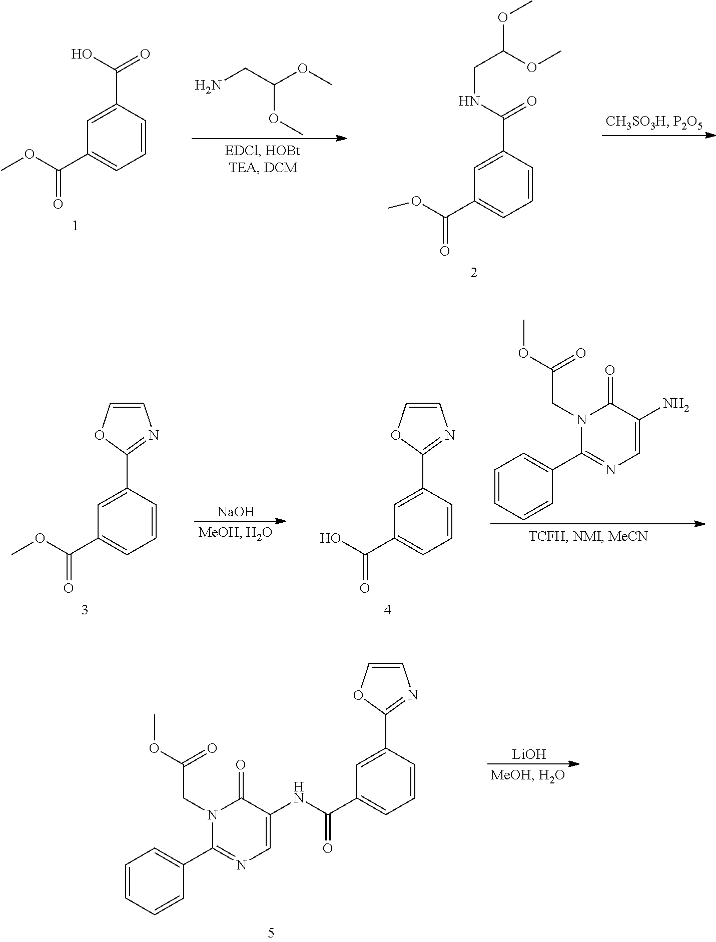

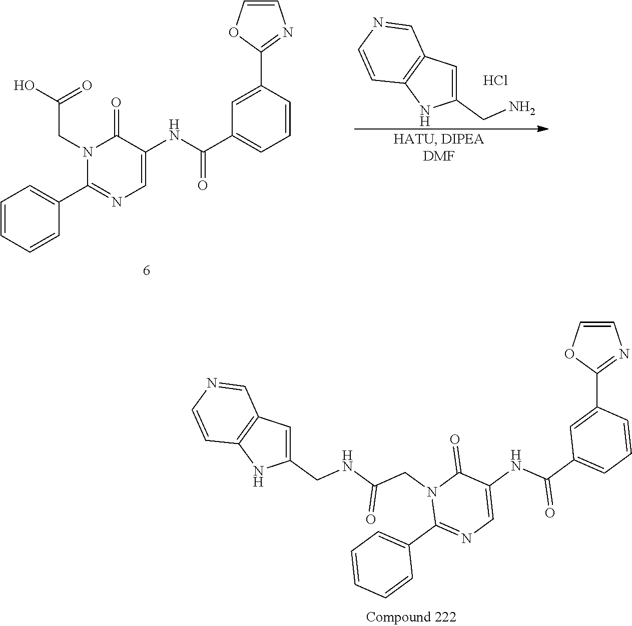 [SMILES: O=C(O)Cn1c(-c2ccccc2)ncc(NC(=O)c2cccc(-c3ncco3)c2)c1=O ; NCc1cc2cnccc2[nH]1 ; Cl ; O=C(Cn1c(-c2ccccc2)ncc(NC(=O)c2cccc(-c3ncco3)c2)c1=O)NCc1cc2cnccc2[nH]1]

### Step 1: Methyl 3-((2,2-dimethoxyethyl)carbamoyl)benzoate (2)

To a mixture of 3-(methoxycarbonyl) benzoic acid (2.3 g, 12.77 mmol) in DCM (200 mL) was added HOBt (2.59 g, 19.16 mmol), EDCl (3.66 g, 19.16 mmol), TEA (3.87 g, 38.31 mmol) and 2,2-dimethoxyethan-1-amine (1.34 g, 12.77 mmol), the reaction mixture was stirred at 25° C. for 16 hours. The mixture was diluted with DCM, washed with brine, dried over anhydrous Na₂SO₄, filtered, and concentrated under reduced pressure to dryness. The residue was purified by flash chromatography (silica gel, 0-30% EtOAc in PE) to give the title compound (3.20 g, yield 93.8%) as a yellow solid. LC/MS (ESI) m/z: 268 (M+H)⁺.

### Step 2: Methyl 3-(oxazol-2-yl) benzoate (3)

To a mixture of methyl 3-((2,2-dimethoxyethyl)carbamoyl)benzoate (3.2 g, 11.9 mmol) in MeSO₃H (50 mL) was added P₂O₅ (10.2 g, 71.8 mmol) and the mixture was stirred at 140° C. for 3 hours. The mixture was quenched with saturated aq.NaHCO₃ solution and extracted with DCM twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (960 mg, yield 39.5%) as a white solid. LC/MS (ESI) m/z: 204 (M+H)⁺.

### Step 3: 3-(Oxazol-2-yl) benzoic acid (4)

To a solution of methyl 3-(oxazol-2-yl) benzoate (500 mg, 2.46 mmol) in MeOH/water (6 mL, v/v/=3/1) was added NaOH (157 mg, 3.94 mmol) and the mixture was stirred at 25° C. for 16 hours. The mixture was acidified with 1 N aq. HCl to pH˜3 and extracted with EtOAc twice. The combined organic layers were concentrated to dryness to give the title compound (400 mg, yield 85.9%) as a white solid, which was used directly in the next step without further purification. LC/MS (ESI) m/z: 190 (M+H)⁺.

### Step 4: Methyl 2-(5-(3-(oxazol-2-yl) benzamido)-6-oxo-2-phenylpyrimidin-1(6H)-yl) acetate (5)

To a solution of 3-(oxazol-2-yl) benzoic acid (131 mg, 0.579 mmol) in MeCN (5 mL) was added methyl 2-(5-amino-6-oxo-2-phenylpyrimidin-1(6H)-yl)acetate (150 mg, 0.579 mmol), NMI (142 mg, 1.74 mmol) and TCFH (488 mg, 1.74 mmol), the reaction mixture was stirred at 25° C. for 16 hours. The mixture was diluted with EtOAc, washed with saturated aq.NaHCO₃ solution and brine, dried over anhydrous Na₂SO₄, filtered, and concentrated under reduced pressure to dryness. The residue was purified by flash chromatography (silica gel, 0-80% EtOAc in PE) to give the title compound (220 mg, yield 88.3%) as a yellow oil. LC/MS (ESI) m/z: 431 (M+H)⁺.

### Step 5: 2-(5-(3-(oxazol-2-yl) benzamido)-6-oxo-2-phenylpyrimidin-1(6H)-yl)acetic acid (6)

To a solution of methyl 2-(5-(3-(oxazol-2-yl) benzamido)-6-oxo-2-phenylpyrimidin-1(6H)-yl) acetate (220 mg, 0.511 mmol) in MeOH/water (4 mL, 3/1) was added LiOH (36.7 mg, 1.53 mmol) and the mixture was stirred at 25° C. for 16 hours. The mixture was acidified with 1N aq. HCl to pH˜6 and extracted with EtOAc twice. The combined organic layers were concentrated to dryness to give the title compound (150 mg, yield 70.5%) as a white solid, which was used in next step without further purification. LC/MS (ESI) m/z: 417 (M+H)⁺.

### Step 6: N-(1-(2-(((1H-pyrrolo[3,2-c]pyridine-2-yl)methyl)amino)-2-oxoethyl)-6-oxo-2-phenyl-1,6-dihydropyrimidin-5-yl)-3-(oxazol-2-yl)benzamide (Compound 222)

To a solution of 2-(5-(3-(oxazol-2-yl) benzamido)-6-oxo-2-phenylpyrimidin-1(6H)-yl)acetic acid (150 mg, 0.360 mmol) in DMF (10 mL) was added HATU (178 mg, 0.468 mmol) and DIPEA (139 mg, 1.08 mmol), (1H-pyrrolo[3,2-c]pyridine-2-yl)methanamine hydrochloride (165 mg, 0.90 mmol) and the reaction mixture was stirred at 25° C. for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-100% EtOAc in PE) and further purified by prep-HPLC to give Compound 222 (35 mg, yield 17.8%) as a white solid. ¹H NMR (400 MHz, DMSO-d₆) δ 11.40 (s, 1H), 9.90 (s, 1H), 8.80 (s, 2H), 8.77 (s, 1H), 8.56 (d, J=1.4 Hz, 1H), 8.31 (s, 1H), 8.21 (d, J=7.8 Hz, 1H), 8.14-8.10 (m, 2H), 7.74-7.70 (m, 1H), 7.60-7.57 (m, 2H), 7.56-7.52 (m, 1H), 7.50-7.45 (m, 3H), 7.34 (d, J=5.4 Hz, 1H), 6.35 (s, 1H), 4.60 (s, 2H), 4.44 (d, J=5.5 Hz, 2H). LC/MS (ESI) m/z: 546 (M+H)⁺. RT (Method A): 1.04 min.

### Scheme 71. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyl-1,3,4-oxadiazole-2-carboxamide (Compound 223)

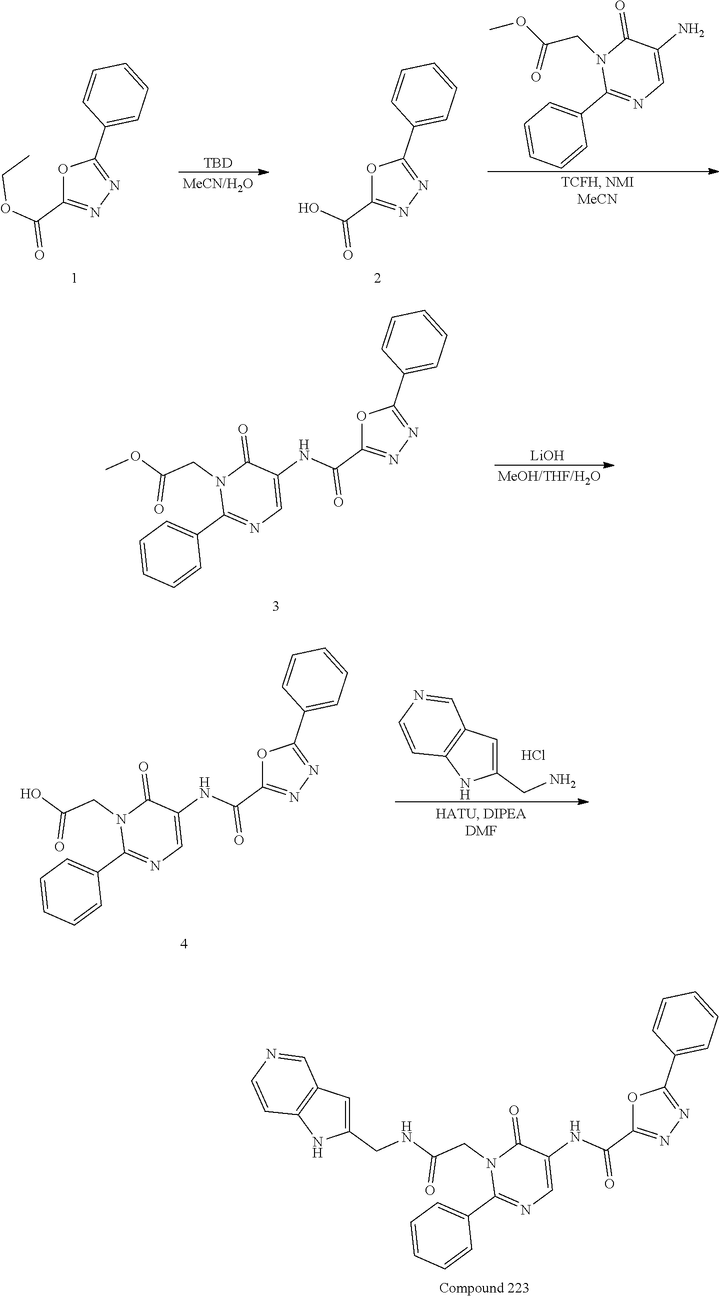

### Step 1: 5-Phenyl-1,3,4-oxadiazole-2-carboxylic acid (2)

To a solution of ethyl 5-phenyl-1,3,4-oxadiazole-2-carboxylate (90 mg, 0.41 mmol) in MeCN (2 mL) was added TBD (12 mg, 0.30 mmol) at room temperature for 2 hours. The reaction mixture was concentrated to dryness under reduced pressure to give the title compound (78 mg, yield 99.9%) as a yellow oil, which was used directly in the next step without further purification. LC/MS (ESI) m/z: 191 (M+H)⁺.

### Step 2: Methyl 2-(6-oxo-2-phenyl-5-(5-phenyl-1,3,4-oxadiazole-2-carboxamido) pyrimidin-1(6H)-yl)acetate (3)

To a mixture of 5-phenyl-1,3,4-oxadiazole-2-carboxylic acid (78 mg, 0.41 mmol) and methyl 2-(5-amino-6-oxo-2-phenylpyrimidin-1(6H)-yl)acetate (106 mg, 0.41 mmol) in MeCN (2 mL) was added NMI (101 mg, 1.23 mmol) and TCFH (344 mg, 1.23 mmol) at 0° C. under N₂ atmosphere and the mixture was stirred at room temperature for 1 hour. The mixture was quenched with saturated aq.NaHCO₃ solution and extracted with CHCl₃/i-PrOH (3/1, v/v) twice. The combined organic layers were washed with brine, dried over anhydrous Na₂SO₄, filtered, and concentrated under reduced pressure to dryness. The residue was purified by flash chromatography (silica gel, 0-60% EtOAc in PE) to give the title compound (145 mg, yield 82.1%) as a yellow oil. LC/MS (ESI) (m/z): 432 (M+H)⁺.

### Step 3: 2-(6-Oxo-2-phenyl-5-(5-phenyl-1,3,4-oxadiazole-2-carboxamido)pyrimidin-1(6H)-yl)acetic acid (4)

To a solution of methyl 2-(6-oxo-2-phenyl-5-(5-phenyl-1,3,4-oxadiazole-2-carboxamido)pyrimidin-1(6H)-yl)acetate (135 mg, 0.31 mmol) in MeOH/THF/water (4 mL, 2/1/1) was added LiOH (39 mg, 0.94 mmol) and the mixture was stirred at room temperature for 1 hour. The mixture was acidified with 1N aq. HCl to pH˜3 and extracted with EtOAc twice. The combined organic layers were dried over anhydrous Na₂SO₄, filtered, and concentrated under reduced pressure to give the title compound (130 mg, yield 99.9%) as a yellow solid, which was used directly in the next step without further purification. LC/MS (ESI) m/z: 418 (M+H)⁺.

### Step 4: N-(1-(2-(((1H-pyrrolo[3,2-c]pyridine-2-yl)methyl)amino)-2-oxoethyl)-6-oxo-2-phenyl-1,6-dihydropyrimidin-5-yl)-5-phenyl-1,3,4-oxadiazole-2-carboxamide (Compound 223)

To a mixture of 2-(6-oxo-2-phenyl-5-(5-phenyl-1,3,4-oxadiazole-2-carboxamido) pyrimidin-1(6H)-yl)acetic acid (125 mg, 0.29 mmol) and (1H-pyrrolo[3,2-c]pyridine-2-yl)methanamine hydrochloride (157 mg, 0.85 mmol) in DMF (3 mL) was added DIPEA (193 mg, 1.5 mmol) and HATU (148 mg, 0.39 mmol) under N₂ atmosphere and the reaction mixture was stirred at room temperature for 2 hours. The mixture was diluted with EtOAc, washed with saturated aq.NaHCO₃ solution and brine, dried over anhydrous Na₂SO₄, filtered, and concentrated under reduced pressure to dryness. The residue was purified by flash chromatography (silica gel, 0-11% MeOH in DCM) and further purified by prep-HPLC to give Compound 223 (22.5 mg, yield 13.7%) as a white solid. ¹H NMR (400 MHz, DMSO-d₆) δ 11.46 (s, 1H), 10.03-9.94 (m, 1H), 8.82 (s, 1H), 8.80 (s, 1H), 8.79 (s, 1H), 8.14 (d, J=5.0 Hz, 2H), 8.13 (s, 1H), 7.73-7.65 (m, 3H), 7.60-7.54 (m, 3H), 7.51-7.46 (m, 2H), 7.38 (d, J=5.5 Hz, 1H), 6.36 (s, 1H), 4.62 (s, 2H), 4.45 (d, J=5.5 Hz, 2H). LC/MS (ESI) m/z: 547 (M+H)⁺. RT (Method A): 1.20 min.

### Scheme 72. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-2-(pyridine-3-yl)oxazole-4-carboxamide (Compound 231)

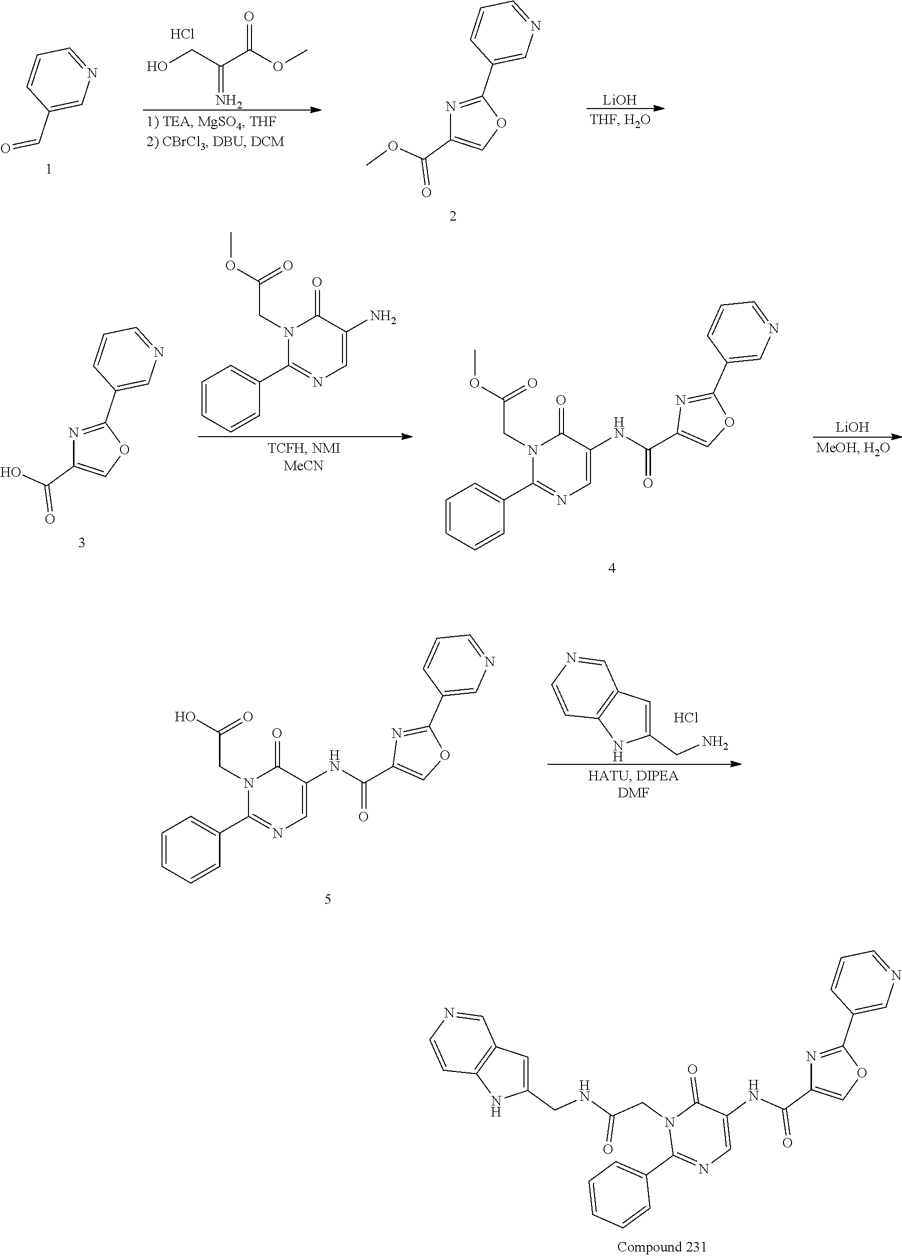

### Step 1: Methyl 2-(pyridine-3-yl)oxazole-4-carboxylate (2)

To a mixture of nicotinaldehyde (2.0 g, 18.7 mmol) and methyl L-serinate hydrochloride (5.8 g, 37.4 mmol) in THF (25 mL) was added MgSO₄ (2.2 g, 18.7 mmol), TEA (3.7 g, 37.4 mmol) under N₂ atmosphere and the reaction mixture was stirred at 25° C. overnight. The mixture was filtered, and the filtrate was concentrated under reduced pressure to dryness. The mixture was diluted with DCM (25 mL) and DBU (8.5 g, 56.1 mmol), CBrCl₃ (7.4 g, 56.1 mmol) were added. The resulting mixture was stirred at 25° C. overnight. The mixture was diluted with EtOAc, washed with brine, dried over anhydrous Na₂SO₄, filtered, and concentrated under reduced pressure to dryness. The residue was purified by flash chromatography (silica gel, 0-28% EtOAc in PE) to give the title compound (1.3 g, yield 34.2%) as a white solid. LC/MS (ESI) m/z: 205 (M+H)⁺.

### Step 2: 2-(Pyridin-3-yl)oxazole-4-carboxylic acid (3)

To a solution of methyl 2-(pyridine-3-yl)oxazole-4-carboxylate (1.3 g, 6.37 mmol) in THF (12 mL) and water (4 mL) was added LiOH (401 mg, 9.56 mmol) and the mixture was stirred at room temperature overnight. The mixture was acidified with 1N aq. HCl to pH˜4 and extracted with EtOAc twice. The combined organic layers were washed with brine, dried over anhydrous Na₂SO₄, filtered, and concentrated under reduced pressure to dryness to give the title compound (1.1 g, yield 90.9%) as a white solid, which was used directly in the next step without further purification. LC/MS (ESI) m/z: 191 (M+H)⁺.

### Step 3: Methyl 2-(6-oxo-2-phenyl-5-(2-(pyridine-3-yl)oxazole-4-carboxamido) pyrimidin-1(6H)-yl)acetate (4)

To a mixture of methyl 2-(5-amino-6-oxo-2-phenylpyrimidin-1(6H)-yl)acetate (130 mg, 0.50 mmol) and 2-(174yridine-3-yl)oxazole-4-carboxylic acid (143 mg, 0.75 mmol) in MeCN (5 mL) was added NMI (247 mg, 3.00 mmol) and TCFH (423 mg, 1.50 mmol). The reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-4% MeOH in DCM) to give the title compound (200 mg, yield 92.5%) as a yellow solid. LC/MS (ESI) m/z: 432 (M+H)⁺.

### Step 4: 2-(6-Oxo-2-phenyl-5-(2-(pyridine-3-yl)oxazole-4-carboxamido)pyrimidin-1(6H)-yl)acetic acid (5)

To a solution of methyl 2-(6-oxo-2-phenyl-5-(2-(pyridine-3-yl)oxazole-4-carboxamido) pyrimidin-1(6H)-yl)acetate (200 mg, 0.46 mmol) in MeOH (3 mL) and water (1 mL) was added LiOH (58 mg, 1.38 mmol) and the mixture was stirred at room temperature for 2 hours. The mixture was acidified with 1 N aq. HCl to pH˜4 and extracted with EtOAc twice. The combined organic layers were washed with brine, dried over anhydrous Na₂SO₄, filtered, and concentrated under reduced pressure to dryness to give the title compound (130 mg, yield 67.2%) as a white solid, which was used directly in the next step without further purification. LC/MS (ESI) m/z: 418 (M+H)⁺.

### Step 5: N-(1-(2-(((1H-pyrrolo[3,2-c]pyridine-2-yl)methyl)amino)-2-oxoethyl)-6-oxo-2-phenyl-1,6-dihydropyrimidin-5-yl)-2-(174yridine-3-yl)oxazole-4-carboxamide (Compound 231)

To a mixture of 2-(6-oxo-2-phenyl-5-(2-(pyridine-3-yl)oxazole-4-carboxamido)pyrimidin-1(6H)-yl)acetic acid (130 mg, 0.31 mmol) and (1H-pyrrolo[3,2-c]pyridine-2-yl)methanamine hydrochloride (115 mg, 0.78 mmol) in DMF (3 mL) was added DIPEA (241 mg, 1.87 mmol) and HATU (142 mg, 0.37 mmol). The mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) and further purified by prep-HPLC to give Compound 231 (35 mg, yield 20.6%) as a white solid. ¹H NMR (400 MHz, DMSO-d₆) δ 11.32 (s, 1H), 9.48 (s, 1H), 9.24 (d, J=2.1 Hz, 1H), 9.10 (s, 1H), 8.98 (s, 1H), 8.81-8.79 (m, 1H), 8.76 (d, J=9.4 Hz, 2H), 8.45-8.42 (m, 1H), 8.11 (d, J=5.6 Hz, 1H), 7.67-7.64 (m, 1H), 7.58-7.54 (m, 3H), 7.48 (d, J=7.3 Hz, 2H), 7.33 (d, J=5.6 Hz, 1H), 6.35 (s, 1H), 4.61 (s, 2H), 4.45 (d, J=5.5 Hz, 2H). LC/MS (ESI) (m/z): 547 (M+H)⁺. RT (Method A): 0.92 min.

### Scheme 73. Synthesis of N-(1-(2-(((1H-pyrrolo[3,2-c]pyridine-2-yl)methyl)amino)-2-oxoethyl)-6-oxo-2-phenyl-1,6-dihydropyrimidin-5-yl)-4-(1H-imidazol-5-yl)benzamide (Compound 174)

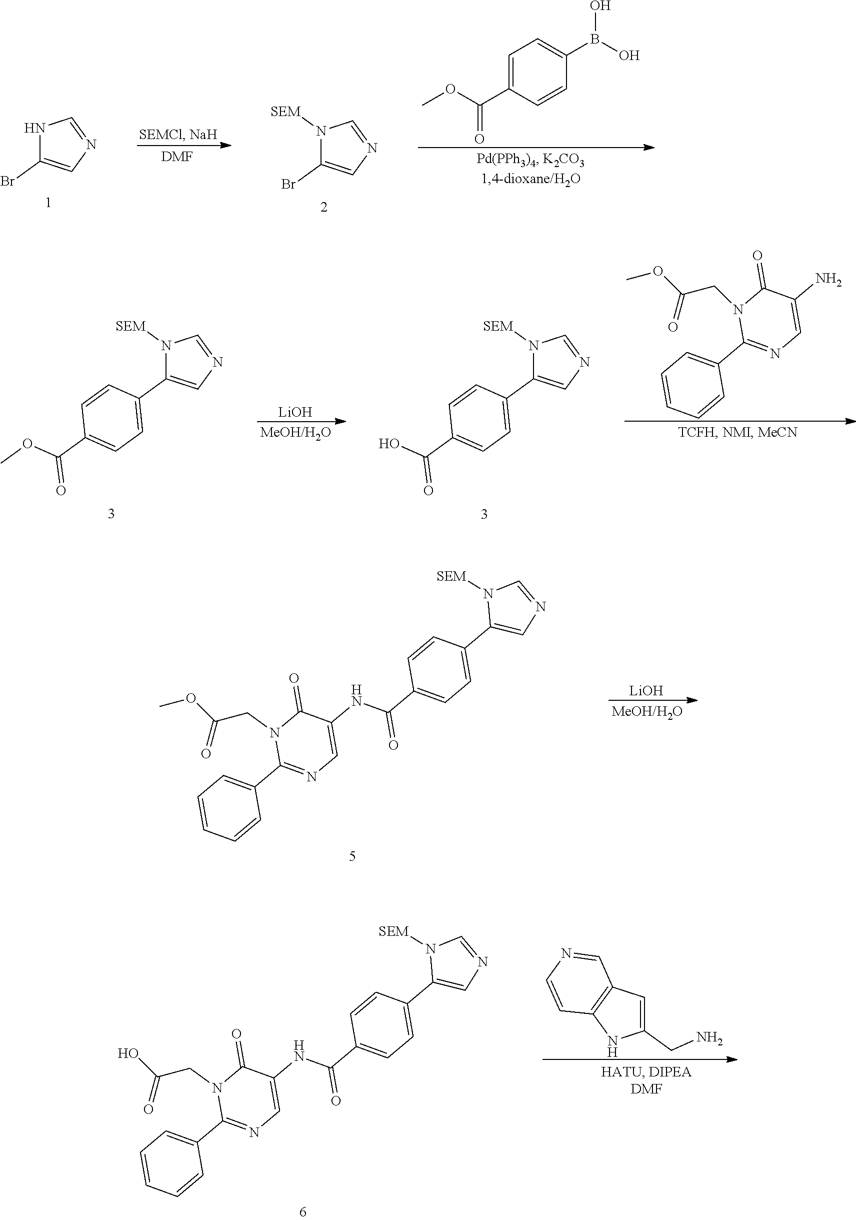

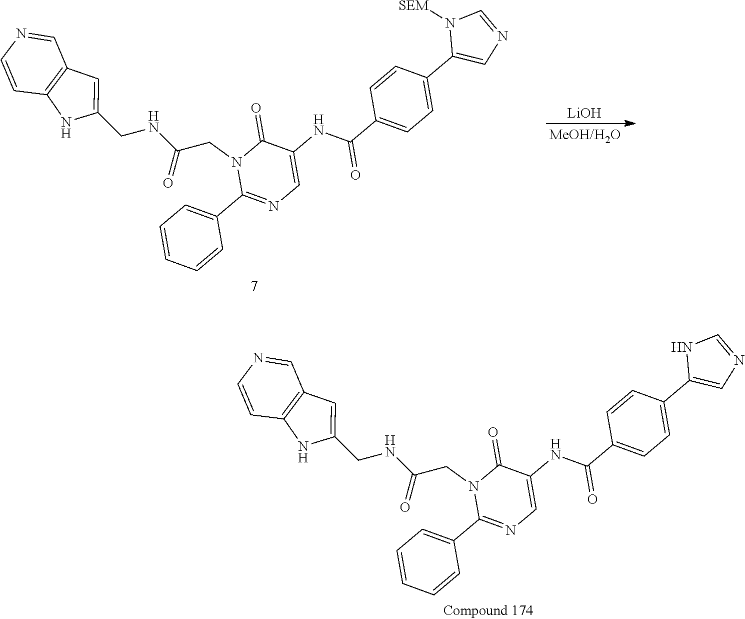 [SMILES: C[Si](C)(C)CCOCn1cncc1-c1ccc(C(=O)Nc2cnc(-c3ccccc3)n(CC(=O)NCc3cc4cnccc4[nH]3)c2=O)cc1 ; "LiOH" ; O=C(Cn1c(-c2ccccc2)ncc(NC(=O)c2ccc(-c3cnc[nH]3)cc2)c1=O)NCc1cc2cnccc2[nH]1]

### Step 1: 5-Bromo-1-((2-(trimethylsilyl)ethoxy)methyl)-1H-imidazole (2)

To solution of 5-bromo-1H-imidazole (2.0 g, 13.6 mmol) in DMF (2 mL) was added NaH (817 mg, 20.4 mmol, 60% dispersion in mineral oil) in portions at 0° C. followed by drop-wise addition of SEMCl (2.9 g, 17.6 mmol) and the reaction mixture was stirred at 80° C. overnight. The mixture was quenched with saturated aq.NH₄Cl solution at 0° C. and extracted with EtOAc twice. The combined organic layers were washed with water and brine, dried over anhydrous Na₂SO₄, filtered, and concentrated under reduced pressure to dryness to give the title compound (3.0 g, yield 79.5%) as a light-yellow oil, which was used directly in the next step without further purification. LC/MS (ESI) m/z: 277 (M+H)⁺.

### Step 2: Methyl 4-(1-((2-(trimethylsilyl)ethoxy)methyl)-1H-imidazol-5-yl)benzoate (3)

To a mixture of 5-bromo-1-((2-(trimethylsilyl)ethoxy)methyl)-1H-imidazole (3.0 g, 10.8 mmol) and (4-(methoxycarbonyl)phenyl)boronic acid (3.6 g, 16.2 mmol) in 1,4-dioxane (30 mL) and water (10 mL) was added K₂CO₃ (5.6 g, 32.4 mmol) and Pd(PPh₃)₄ (1.5 g, 1.1 mmol) under N₂ atmosphere, the reaction mixture was degassed under N₂ atmosphere for three times and stirred at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% MeOH in DCM) to give the title compound (1.9 g, yield 52.8%) as a colorless oil. LC/MS (ESI) m/z: 333 (M+H)⁺.

### Step 3: 4-(1-((2-(Trimethylsilyl)ethoxy)methyl)-1H-imidazol-5-yl)benzoic acid (4)

To a solution of methyl 4-(1-((2-(trimethylsilyl)ethoxy)methyl)-1H-imidazol-5-yl)benzoate (500 mg, 1.50 mmol) in MeOH/water (10 mL, v/v=2/1) was added LiOH·H₂O (81 mg, 1.95 mmol) under N₂ atmosphere and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (240 mg, yield 50.1%) as a yellow solid, which was used directly in the next step without further purification. LC/MS (ESI) m/z: 319 (M+H)⁺.

### Step 4: Methyl 2-(6-oxo-2-phenyl-5-(4-(1-((2-(trimethylsilyl)ethoxy)methyl)-1H-imidazol-5-yl)benzamido)pyrimidin-1(6H)-yl)acetate (5)

To a mixture of 4-(1-((2-(trimethylsilyl)ethoxy)methyl)-1H-imidazol-5-yl)benzoic acid (240 mg, 0.75 mmol) and methyl 2-(5-amino-6-oxo-2-phenylpyrimidin-1(6H)-yl)acetate (130 mg, 0.50 mmol) in MeCN (2 mL) was added TCFH (422 mg, 1.51 mmol) and NMI (123 mg, 1.51 mmol) under N₂ atmosphere and the reaction mixture was stirred at room temperature for 2 hours. The mixture was diluted with EtOAc, washed with saturated aq.NaHCO₃ solution and brine, dried over anhydrous Na₂SO₄, filtered, and concentrated under reduced pressure to dryness. The residue was purified by flash chromatography (silica gel, 0-5% MeOH in DCM) to give the title compound (171 mg, yield 60.8%) as a white solid. LC/MS (ESI) m/z: 560 (M+H)⁺.

### Step 5: 2-(6-Oxo-2-phenyl-5-(4-(1-((2-(trimethylsilyl)ethoxy)methyl)-1H-imidazol-5-yl)benzamido)pyrimidin-1(6H)-yl)acetic acid (6)

To a solution of methyl 2-(6-oxo-2-phenyl-5-(4-(1-((2-(trimethylsilyl)ethoxy)methyl)-1H-imidazol-5-yl)benzamido)pyrimidin-1(6H)-yl)acetate (171 mg, 0.31 mmol) in MeOH/water (3 mL, v/v=2/1) was added LiOH·H₂O (17 mg, 0.39 mmol) under N₂ atmosphere and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (94 mg, yield 99.4%) as a yellow solid, which was used directly in the next step without further purification. LC/MS (ESI) m/z: 546 (M+H)⁺.

### Step 6: N-(1-(2-(((1H-pyrrolo[3,2-c]pyridine-2-yl)methyl)amino)-2-oxoethyl)-6-oxo-2-phenyl-1,6-dihydropyrimidin-5-yl)-4-(1-((2-(trimethylsilyl)ethoxy)methyl)-1H-imidazol-5-yl)benzamide (7)

To a mixture of 2-(6-oxo-2-phenyl-5-(4-(1-((2-(trimethylsilyl)ethoxy)methyl)-1H-imidazol-5-yl)benzamido)pyrimidin-1(6H)-yl)acetic acid (94 mg, 0.17 mmol) and (1H-pyrrolo[3,2-c]pyridine-2-yl)methanamine hydrochloride (38 mg, 0.25 mmol) in DMF (2 mL) was added DIPEA (89 mg, 0.69 mmol) and HATU (85 mg, 0.22 mmol) under N₂ atmosphere and the reaction mixture was stirred at room temperature for 2 hours. The mixture was diluted with EtOAc, washed with saturated aq.NaHCO₃ solution and brine, dried over anhydrous Na₂SO₄, filtered, and concentrated under reduced pressure to dryness. The residue was purified by flash chromatography (silica gel, 0-20% MeOH in DCM) to give the title compound (101 mg, yield 86.8%) as a white solid. LC/MS (ESI) m/z: 675 (M+H)⁺.

### Step 7: N-(1-(2-(((1H-pyrrolo[3,2-c]pyridine-2-yl)methyl)amino)-2-oxoethyl)-6-oxo-2-phenyl-1,6-dihydropyrimidin-5-yl)-4-(1H-imidazol-5-yl)benzamide (Compound 174)

To a solution of N-(1-(2-(((1H-pyrrolo[3,2-c]pyridine-2-yl)methyl)amino)-2-oxoethyl)-6-oxo-2-phenyl-1,6-dihydropyrimidin-5-yl)-4-(1-((2-(trimethylsilyl)ethoxy)methyl)-1H-imidazol-5-yl)benzamide (101 mg, 0.15 mmol) in MeOH/water (3 mL, v/v=2/1) was added LiOH·H₂O (8.1 mg, 0.19 mmol) and the reaction mixture was stirred at room temperature for 2 hours. The mixture was acidified with 1 N aq. HCl to pH˜3 and extracted with EtOAc twice. The combined organic layers were dried over anhydrous Na₂SO₄, filtered, and concentrated under reduced pressure to dryness to give Compound 174 (13.7 mg, yield 16.8%) as a white solid, which was used directly in the next step without further purification. ¹H NMR (400 MHz, DMSO-d₆) δ 12.32 (s, 1H), 11.34 (s, 1H), 9.47 (d, J=34.0 Hz, 1H), 8.89-8.70 (m, 3H), 8.12 (d, J=5.6 Hz, 1H), 8.06-7.88 (m, 4H), 7.79 (d, J=18.7 Hz, 2H), 7.59-7.53 (m, 3H), 7.47 (t, J=7.3 Hz, 2H), 7.32 (d, J=5.6 Hz, 1H), 6.33 (s, 1H), 4.59 (s, 2H), 4.43 (d, J=5.5 Hz, 2H). LC/MS (ESI) m/z: 545 (M+H)⁺. RT (Method A): 0.32 min.

### Scheme 74. Synthesis of N-(1-(2-(((1H-pyrrolo[3,2-c]pyridine-2-yl)methyl)amino)-2-oxoethyl)-2-(4-(((3R,3aR,6R,6aR)-6-methoxyhexahydrofuro[3,2-b]furan-3-yl)oxy) phenyl)-6-oxo-1,6-dihydropyrimidin-5-yl)-5-phenylthiophene-2-carboxamide (Compound 249)

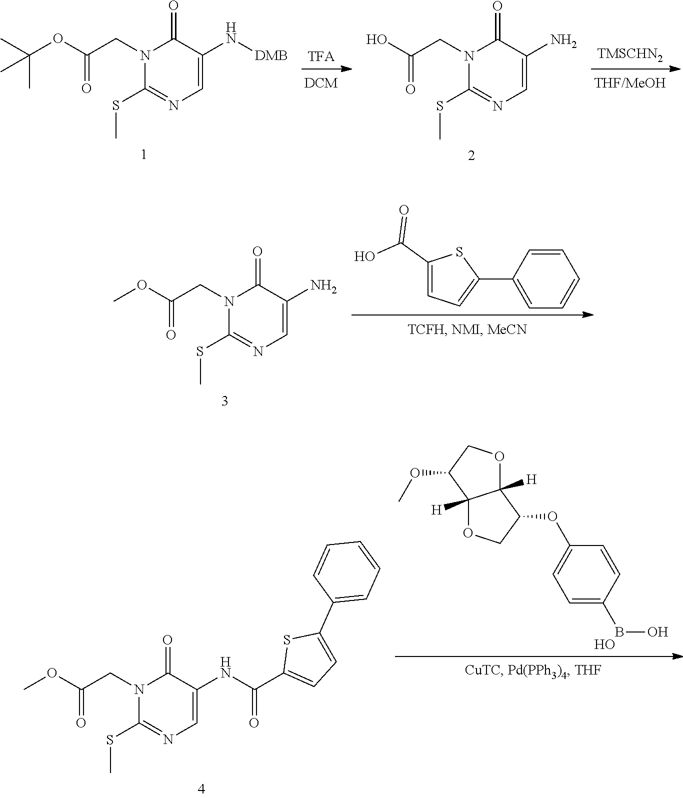

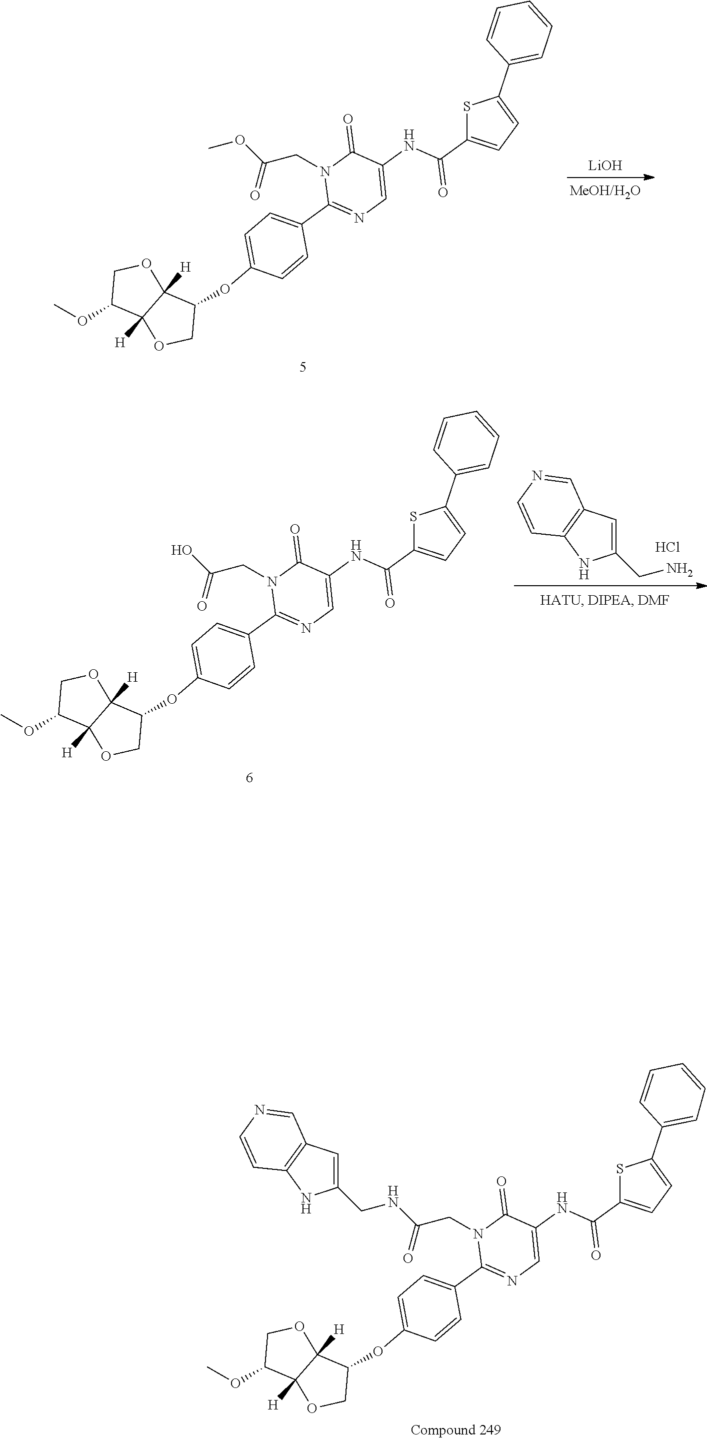

### Step 1: 2-(5-Amino-2-(methylthio)-6-oxopyrimidin-1(6H)-yl)acetic acid (2)

To a solution of tert-butyl 2-(5-((2,4-dimethoxybenzyl)amino)-2-(methylthio)-6-oxopyrimidin-1(6H)-yl)acetate (2.0 g, 4.75 mmol) in DCM (20 mL) was added TEA (20 mL) and the mixture was stirred at room temperature for 2 hours. The reaction mixture was concentrated under reduced pressure to dryness to give the title compound (1 g, yield 97.5%) as a yellow solid, which was used directly in the next step without further purification. LC/MS (ESI) m/z: 216 (M+H)⁺.

### Step 2: Methyl 2-(5-amino-2-(methylthio)-6-oxopyrimidin-1(6H)-yl)acetate (3)

To a solution of 2-(5-amino-2-(methylthio)-6-oxopyrimidin-1(6H)-yl)acetic acid (1.0 g, 4.65 mmol) in THF (5 mL) and MeOH (5 mL) was added TMSCHN₂ (5 mL, 2M in hexane) drop-wisely at 0° C. and the mixture was stirred at room temperature for 3 hours. The mixture was diluted with DCM, washed with saturated aq.NaHCO₃ solution and brine, dried over anhydrous Na₂SO₄, filtered, and concentrated under reduced pressure to dryness. The residue was purified by flash chromatography (silica gel, 0-5% MeOH in DCM) to give the title compound (360 mg, yield 33.6%) as a yellow solid. LC/MS (ESI) m/z: 230 (M+H)⁺.

### Step 3: Methyl 2-(2-(methylthio)-6-oxo-5-(5-phenylthiophene-2-carboxamido) pyrimidin-1(6H)-yl)acetate (4)

To a mixture of methyl 2-(5-amino-2-(methylthio)-6-oxopyrimidin-1(6H)-yl)acetate (360 mg, 1.57 mmol) and 5-phenylthiophene-2-carboxylic acid (417 mg, 2.04 mmol) in MeCN (4 mL) was added NMI (377 mg, 4.71 mmol) and TCEH (1.32 g, 4.71 mmol), the mixture was stirred at room temperature for 1 hour. The mixture was diluted with DCM, washed with saturated aq.NaHCO₃ solution and brine, dried over anhydrous Na₂SO₄, filtered, and concentrated under reduced pressure to dryness. The residue was purified by flash chromatography (silica gel, 0-5% MeOH in DCM) to give the title compound (320 mg, yield 49.2%) as a yellow solid. LC/MS (ESI) m/z: 416 (M+H)⁺.

### Step 4: Methyl 2-(2-(4-(((3R,3aR,6R,6aR)-6-methoxyhexahydrofuro[3,2-b]furan-3-yl)oxy)phenyl)-6-oxo-5-(5-phenylthiophene-2-carboxamido)pyrimidin-1(6H)-yl) acetate (5)

To a mixture of methyl 2-(2-(methylthio)-6-oxo-5-(5-phenylthiophene-2-carboxamido) pyrimidin-1(6H)-yl)acetate (160 mg, 0.39 mmol) and (4-(((3R,3aR,6R,6aR)-6-methoxyhexahydrofuro[3,2-b]furan-3-yl)oxy)phenyl)boronic acid (162 mg, 0.58 mmol) in THF (5 mL) was added CuTC (162 mg, 0.85 mmol) and Pd(PPh₃)₄ (89 mg, 0.077 mmol) at room temperature under N₂ atmosphere. The reaction mixture was degassed under N₂ atmosphere for three times and stirred at 70° C. overnight. The mixture was diluted with EtOAc and filtered. The filtrate was washed with saturated aq.NaHCO₃ solution and brine, dried over anhydrous Na₂SO₄, filtered, and concentrated under reduced pressure to dryness. The residue was purified by flash chromatography (silica gel, 0-10% MeOH in DCM) to give the title compound (110 mg, yield 46.8%) as a brown solid. LC/MS (ESI) m/z: 604 (M+H)⁺.

### Step 5: 2-(2-(4-(((3R,3aR,6R,6aR)-6-methoxyhexahydrofuro[3,2-b]furan-3-yl)oxy) phenyl)-6-oxo-5-(5-phenylthiophene-2-carboxamido)pyrimidin-1(6H)-yl)acetic acid (6)

To a solution of methyl 2-(2-(4-(((3R,3aR,6R,6aR)-6-methoxyhexahydrofuro[3,2-b]furan-3-yl)oxy)phenyl)-6-oxo-5-(5-phenylthiophene-2-carboxamido)pyrimidin-1(6H)-yl)acetate (110 mg, 0.18 mmol) in MeOH/water (1.25 mL, v/v=4/1) was added LiOH (15 mg, 0.36 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (100 mg, yield 94.3%) as a white solid, which was used directly in the next step without further purification. LC/MS (ESI) m/z: 590 (M+H)⁺.

### Step 6: N-(1-(2-(((1H-pyrrolo[3,2-c]pyridine-2-yl)methyl)amino)-2-oxoethyl)-2-(4-(((3R,3aR,6R,6aR)-6-methoxyhexahydrofuro[3,2-b]furan-3-yl)oxy)phenyl)-6-oxo-1,6-dihydropyrimidin-5-yl)-5-phenylthiophene-2-carboxamide (Compound 249)

To a mixture of 2-(2-(4-(((3R,3aR,6R,6aR)-6-methoxyhexahydrofuro[3,2-b]furan-3-yl)oxy)phenyl)-6-oxo-5-(5-phenylthiophene-2-carboxamido)pyrimidin-1(6H)-yl)acetic acid (100 mg, 0.17 mmol) and (1H-pyrrolo[3,2-c]pyridine-2-yl)methanamine hydrochloride (48 mg, 0.26 mmol) in DMF (2 mL) was added DIPEA (110 mg, 0.85 mmol) and HATU (84 mg, 0.22 mmol), the reaction mixture was stirred at room temperature for 2 hours. The mixture was concentrated under reduced pressure to dryness. The residue was purified by flash chromatography (silica gel, 0-15% MeOH in DCM) and further purified by prep-HPLC to give Compound 249 (22.2 mg, yield 18.2%) as a white solid. ¹H NMR (400 MHz, DMSO-d₆) δ 11.38 (s, 1H), 9.64 (s, 1H), 8.82 (t, J=5.1 Hz, 1H), 8.76 (s, 1H), 8.68 (s, 1H), 8.16-8.13 (m, 1H), 8.11 (d, J=4.1 Hz, 1H), 7.77 (d, J=7.4 Hz, 2H), 7.63 (d, J=4.0 Hz, 1H), 7.51 (d, J=8.8 Hz, 2H), 7.47 (d, J=7.8 Hz, 2H), 7.40 (t, J=7.3 Hz, 1H), 7.35 (d, J=5.7 Hz, 1H), 7.04 (d, J=8.8 Hz, 2H), 6.40 (s, 1H), 4.89-4.84 (m, 1H), 4.77 (t, J=5.0 Hz, 1H), 4.60 (s, 2H), 4.46 (d, J=5.4 Hz, 2H), 4.08-4.04 (m, 1H), 3.93-3.89 (m, 1H), 3.86-3.82 (m, 1H), 3.78 (d, J=2.7 Hz, 1H), 3.46 (t, J=8.2 Hz, 2H), 3.35 (s, 3H). LC/MS (ESI) m/z: 719 (M+H)⁺. RT (Method A): 1.50 min.

### Scheme 75. Synthesis of N-((1H-pyrrolo[3,2-c]pyridin-2-yl)methyl)-2-(5-(((2′-fluoro-[1,1′-biphenyl]-4-yl)methyl)amino)-6-oxo-2-phenylpyrimidin-1(6H)-yl)acetamide (Compound 250)

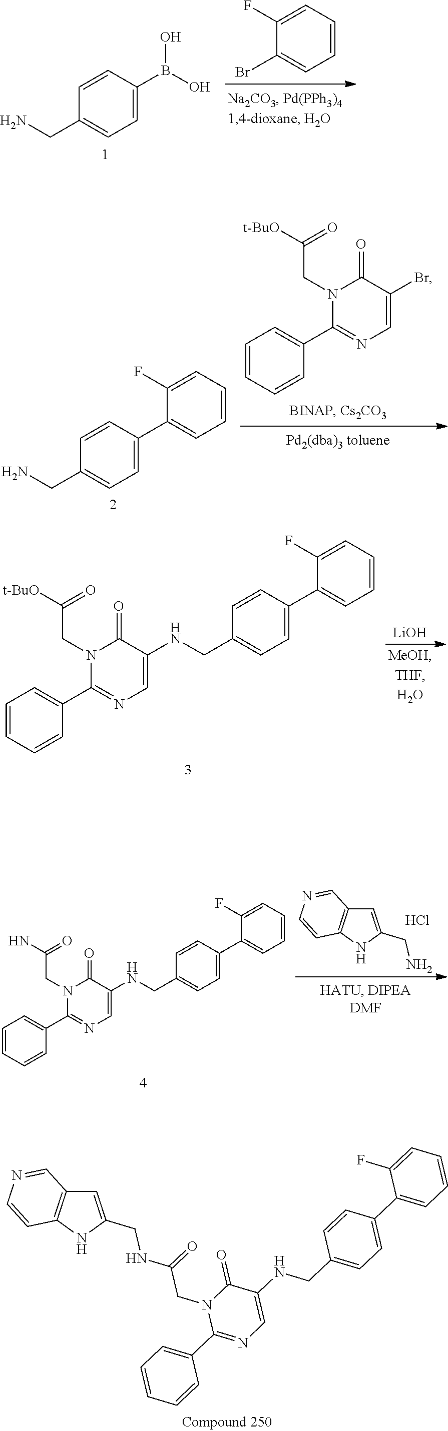

### Step 1: (2′-Fluoro-[1,1′-biphenyl]-4-yl)methanamine (2)

To a mixture of 1-bromo-2-fluorobenzene (500 mg, 2.86 mmol) and (4-(aminomethyl)phenyl)boronic acid (696 mg, 3.71 mmol) in 1,4-dioxane (6 mL) and water (1 mL) was added Na₂CO₃ (908 mg, 5.57 mmol), Pd(PPh₃)₄ (330 mg, 0.29 mmol) under N₂ atmosphere and the reaction mixture was stirred at 90° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give the title compound (370 mg, yield 64.4%) as a brown solid. LC/MS (ESI) m/z: 202 (M+H)⁺.

### Step 2: Tert-butyl 2-(5-(((2′-fluoro-[1,1′-biphenyl]-4-yl)methyl)amino)-6-oxo-2-phenylpyrimidin-1(6H)-yl)acetate (3)

To a mixture of tert-butyl 2-(5-bromo-6-oxo-2-phenylpyrimidin-1(6H)-yl)acetate (200 mg, 0.55 mmol) and (2′-fluoro-[1,1′-biphenyl]-4-yl)methanamine (133 mg, 0.66 mmol) in toluene (3 mL) was added Cs₂CO₃ (537 mg, 1.65 mmol), BINAP (34 mg, 0.06 mmol), Pd₂(dba)₃ (50 mg, 0.06 mmol) under N₂ atmosphere, the reaction mixture was degassed under N₂ atmosphere for three times and stirred at 100° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-18% EtOAc in PE) to give the title compound (240 mg, yield 90.2%) as orange solid. LC/MS (ESI) m/z: 486 (M+H)⁺.

### Step 3: 2-(5-(((2′-fluoro-[1,1′-biphenyl]-4-yl)methyl)amino)-6-oxo-2-phenylpyrimidin-1(6H)-yl)acetic acid (4)

To a solution of tert-butyl 2-(5-(((2′-fluoro-[1,1′-biphenyl]-4-yl)methyl)amino)-6-oxo-2-phenylpyrimidin-1(6H)-yl)acetate (240 mg, 0.49 mmol) in MeOH (2 mL), THF (2 mL) and water (2 mL) was added LiOH (36 mg, 1.47 mmol) and the mixture was stirred at 50° C. overnight. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (210 mg, yield 99.1%) as a brown solid, which was used directly in the next step without further purification. LC/MS (ESI) m/z: 430 (M+H)⁺.

### Step 4: N-((1H-pyrrolo[3,2-c]pyridin-2-yl)methyl)-2-(5-(((2′-fluoro-[1,1′-biphenyl]-4-yl)methyl)amino)-6-oxo-2-phenylpyrimidin-1(6H)-yl)acetamide (Compound 250)

To a mixture of 2-(5-(((2′-fluoro-[1,1′-biphenyl]-4-yl)methyl)amino)-6-oxo-2-phenylpyrimidin-1(6H)-yl)acetic acid (200 mg, 0.46 mmol) and (1H-pyrrolo[3,2-c]pyridine-2-yl)methanamine hydrochloride (136 mg, 0.74 mmol) in DMF (3 mL) was added DIPEA (360 mg, 2.76 mmol) and HATU (212 mg, 0.55 mmol) at 0° C. and the mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give the title compound (40 mg, yield 15.4%) as a white solid. ¹H NMR (400 MHz, CD₃OD) δ 8.93 (s, 1H), 8.44 (s, 1H), 8.24 (d, J=6.4 Hz, 1H), 7.73 (d, J=6.4 Hz, 1H), 7.54-7.51 (m, 2H), 7.49 (d, J=2.6 Hz, 3H), 7.47 (d, J=2.0 Hz, 2H), 7.45 (s, 1H), 7.41 (d, J=7.2 Hz, 2H), 7.37-7.33 (m, 1H), 7.25 (d, J=7.5 Hz, 1H), 7.20-7.15 (m, 1H), 7.07 (s, 1H), 6.76 (s, 1H), 4.66 (s, 2H), 4.61 (s, 2H), 4.49 (s, 2H). LC/MS (ESI) (m/z): 559 (M+H)⁺. RT (Method A): 1.65 min.

Compound 257 was prepared based on Scheme 75:

| # | Reactant A | Reactant B | Reactant C | Reactant D |
|---|---|---|---|---|
| 257 | 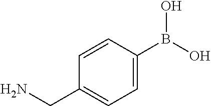 [SMILES: NCc1ccc(B(O)O)cc1] | 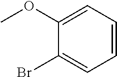 | 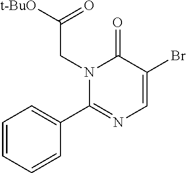 [SMILES: CC(C)(C)OC(=O)Cn1c(-c2ccccc2)ncc(Br)c1=O] | 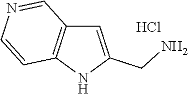 [SMILES: NCc1cc2cnccc2[nH]1 ; Cl] |
| | | | # | Characterization Data |
| | | | 257 | ¹H NMR (400 MHz, DMSO-d₆) δ 11.32 (s, |
| | | | | 1H), 8.74 (s, 1H), 8.69 (t, J = 5.7 Hz, 1H), |
| | | | | 8.12 (d, J = 5.7 Hz, 1H), 7.46-7.44 (m, |
| | | | | 2H), 7.43 (d, J = 3.3 Hz, 5H), 7.39 (t, J = |
| | | | | 6.8 Hz, 2H), 7.35-7.32 (m, 2H), 7.31- |
| | | | | 7.27 (m, 1H), 7.10 (d, J = 7.7 Hz, 1H), 7.06 |
| | | | | (s, 1H), 7.03-6.99 (m, 1H), 6.32 (s, 1H), |
| | | | | 6.29 (t, J = 6.4 Hz, 1H), 4.51 (s, 2H), 4.42 |
| | | | | (d, J = 5.6 Hz, 2H), 4.38 (d, J = 6.4 Hz, |
| | | | | 2H), 3.75 (s, 3H). LCMS (ESI) (m/z): 571 |
| | | | | (M + H)⁺. RT (Method A): 1.65 min. |

### Scheme 76. Synthesis of N-((1H-pyrrolo[3,2-c]pyridine-2-yl)methyl)-2-(2-cyclopentyl-5-((dibenzo[b,d]furan-2-ylmethyl)amino)-6-oxopyrimidin-1(6H)-yl)acetamide (Compound 253)

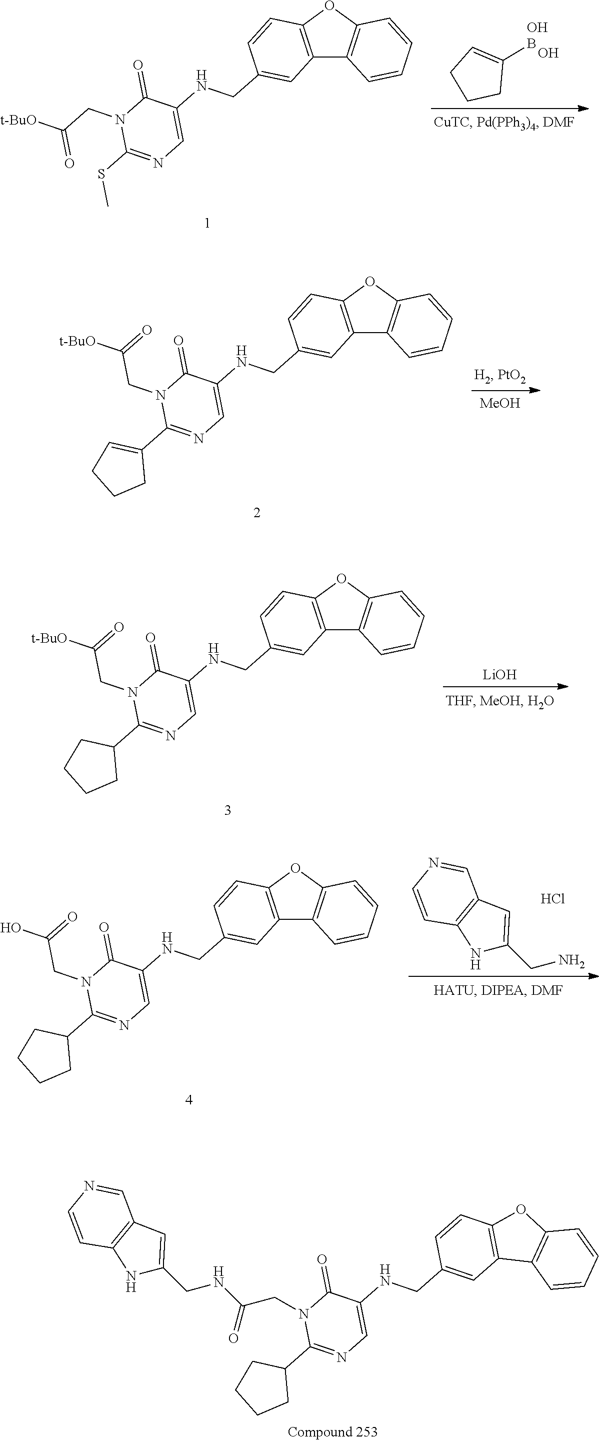

### Step 1: Tert-butyl 2-(2-(cyclopent-1-en-1-yl)-5-((dibenzo[b,d]furan-2-ylmethyl) amino)-6-oxopyrimidin-1(6H)-yl)acetate (2)

To a mixture of tert-butyl 2-(5-((dibenzo[b,d]furan-2-ylmethyl)amino)-2-(methylthio)-6-oxopyrimidin-1(6H)-yl)acetate (100 mg, 0.22 mmol) and cyclopent-1-en-1-ylboronic acid (99 mg, 0.88 mmol) in DMF (1 mL) were added CuTC (93 mg, 0.49 mmol) and Pd(PPh₃)₄ (26 mg, 0.02 mmol) under N₂ atmosphere. The mixture was degassed under N₂ atmosphere for three times and stirred at 80° C. for 16 hours. The reaction mixture was diluted with EtOAc (10 mL), washed with saturated aq.NaHCO₃ solution (10 mL×4) and brine (10 mL), dried over anhydrous Na₂SO₄, filtered, and concentrated under reduced pressure to dryness. The residue was purified by flash chromatography (silica gel, 0-51% EtOAc in PE) to give title compound (77 mg, yield 73.7%) as a yellow solid. LC/MS (ESI) m/z: 472 (M+H)⁺.

### Step 2: Tert-butyl 2-(2-cyclopentyl-5-((dibenzo[b,d]furan-2-ylmethyl)amino)-6-oxopyrimidin-1(6H)-yl)acetate (3)

To a solution of tert-butyl 2-(2-(cyclopent-1-en-1-yl)-5-((dibenzo[b,d]furan-2-ylmethyl) amino)-6-oxopyrimidin-1(6H)-yl)acetate (62 mg, 0.13 mmol) in MeOH (3 mL) was added PtO₂ (30 mg, 0.13 mmol), the mixture was degassed under N₂ atmosphere for three times and stirred under a H₂ balloon at 50° C. overnight. The mixture was filtered, and the filtrate was concentrated to dryness to give the title compound (60 mg, yield 96.4%) as a white solid, which was used directly in the next step without further purification. LC/MS (ESI) m/z: 474 (M+H)⁺.

### Step 3: 2-(2-Cyclopentyl-5-((dibenzo[b,d]furan-2-ylmethyl)amino)-6-oxopyrimidin-1(6H)-yl)acetic acid (4)

To a solution of tert-butyl 2-(2-cyclopentyl-5-((dibenzo[b,d]furan-2-ylmethyl)amino)-6-oxopyrimidin-1(6H)-yl)acetate (30 mg, 0.06 mmol) in MeOH (1 mL), THF (2 mL) and water (0.5 mL) was added LiOH (6 mg, 0.25 mmol) and the mixture was stirred at 60° C. for 2 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (20 mg, yield 75.6%) as a white solid, which was used directly in the next step without further purification. LC/MS (ESI) m/z: 418 (M+H)⁺.

### Step 4: N-((1H-pyrrolo[3,2-c]pyridine-2-yl)methyl)-2-(2-cyclopentyl-5-((dibenzo[b,d]furan-2-ylmethyl)amino)-6-oxopyrimidin-1(6H)-yl)acetamide (Compound 253)

To a mixture of 2-(2-cyclopentyl-5-((dibenzo[b,d]furan-2-ylmethyl)amino)-6-oxopyrimidin-1(6H)-yl)acetic acid (20 mg, 0.05 mmol) and (1H-pyrrolo[3,2-c]pyridine-2-yl)methanamine hydrochloride (13 mg, 0.07 mmol) in DMF (1 mL) was added DIPEA (31 mg, 0.24 mmol) and HATU (22 mg, 0.06 mmol) at 0° C. and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 253 (5.9 mg, yield 22.5%) as a white solid. ¹H NMR (400 MHz, DMSO-d₆) δ 11.36 (s, 1H), 8.82 (t, J=5.5 Hz, 1H), 8.73 (s, 1H), 8.15-8.06 (m, 3H), 7.70-7.63 (m, 2H), 7.56-7.43 (m, 2H), 7.39 (t, J=7.2 Hz, 1H), 7.32 (d, J=5.6 Hz, 1H), 6.95 (d, J=15.7 Hz, 1H), 6.43 (s, 1H), 6.10-5.96 (m, 1H), 4.80 (s, 2H), 4.49-4.40 (m, 4H), 3.02-2.93 (m, 1H), 1.82-1.67 (m, 4H), 1.63-1.44 (m, 4H). LC/MS (ESI) (m/z): 547 (M+H)⁺. RT (Method A): 1.80 min.

The following compounds were prepared based on Scheme 76:

| # | Reactant A | Reactant B | Reactant C |
|---|---|---|---|
| 277 | 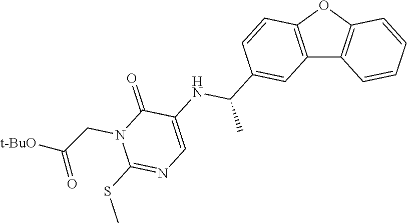 [SMILES: CSc1ncc(N[C@@H](C)c2ccc3oc4ccccc4c3c2)c(=O)n1CC(=O)OC(C)(C)C] | 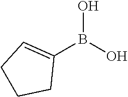 | 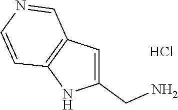 [SMILES: NCc1cc2cnccc2[nH]1 ; Cl] |
| 324 | 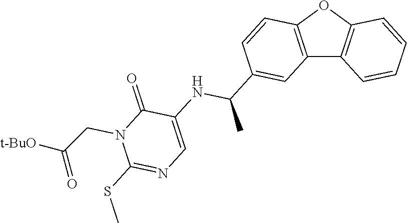 [SMILES: CSc1ncc(N[C@H](C)c2ccc3oc4ccccc4c3c2)c(=O)n1CC(=O)OC(C)(C)C] | 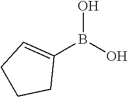 | 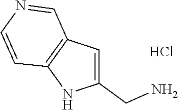 [SMILES: NCc1cc2cnccc2[nH]1 ; Cl] |
| 420^{a} | 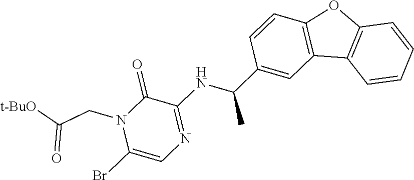 [SMILES: C[C@@H](Nc1ncc(Br)n(CC(=O)OC(C)(C)C)c1=O)c1ccc2oc3ccccc3c2c1] | 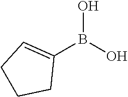 | 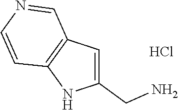 [SMILES: NCc1cc2cnccc2[nH]1 ; Cl] |
| 450 | 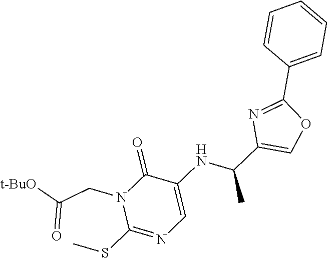 [SMILES: CSc1ncc(N[C@H](C)c2coc(-c3ccccc3)n2)c(=O)n1CC(=O)OC(C)(C)C] | 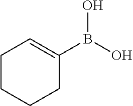 | 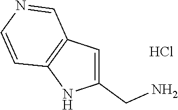 [SMILES: NCc1cc2cnccc2[nH]1 ; Cl] |
| 476 | 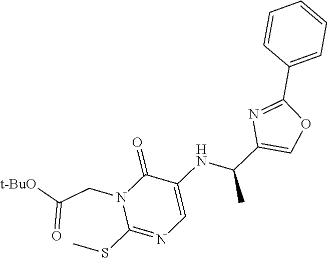 [SMILES: CSc1ncc(N[C@H](C)c2coc(-c3ccccc3)n2)c(=O)n1CC(=O)OC(C)(C)C] | 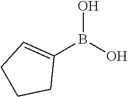 | 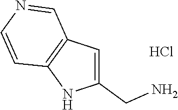 [SMILES: NCc1cc2cnccc2[nH]1 ; Cl] |

| # | Characterization Data |
|---|---|
| 277 | LC/MS (ESI) (m/z): 561 (M + H)⁺. RT (Method A): |
| | 1.89 min. |
| 324 | ¹H NMR (400 MHz, DMSO-d₆) δ 11.36 (s, 1H), |
| | 8.81 (s, 1H), 8.73 (s, 1H), 8.18 (s, 1H), 8.14- |
| | 8.03 (m, 2H), 7.71-7.61 (m, 2H), 7.58 (d, J = |
| | 7.8 Hz, 1H), 7.50 (d, J = 7.6 Hz, 1H), 7.39 (d, J = |
| | 6.7 Hz, 1H), 7.32 (d, J = 4.7 Hz, 1H), 6.84 (s, |
| | 1H), 6.43 (s, 1H), 5.58 (d, J = 6.0 Hz, 1H), 4.90- |
| | 4.70 (m, 2H), 4.56 (s, 1H), 4.47 (s, 2H), 3.02- |
| | 2.83 (m, 1H), 1.85-1.67 (m, 3H), 1.57 (m, 3H), |
| | 1.56-1.49 (m, 3H), 1.48-1.41 (m, 2H). LC/MS |
| | (ESI) (m/z): 561 (M + H)⁺. RT (Method A): 1.87 |
| | min. |
| 420a | ¹H NMR (400 MHz, DMSO-d₆) δ 11.33 (s, 1H), |
| | 8.76 (t, J = 5.7 Hz, 1H), 8.73 (s, 1H), 8.15 (d, J = |
| | 1.4 Hz, 1H), 8.12-8.09 (m, 2H), 7.67 (d, J = |
| | 8.2 Hz, 1H), 7.61 (d, J = 8.5 Hz, 1H), 7.59-7.56 |
| | (m, 1H), 7.52-7.48 (m, 1H), 7.41-7.36 (m, |
| | 1H), 7.32 (d, J = 5.7 Hz, 1H), 7.25 (d, J = 8.1 Hz, |
| | 1H), 6.66 (s, 1H), 6.41 (s, 1H), 5.26-5.18 (m, |
| | 1H), 4.75-4.64 (m, 2H), 4.45 (d, J = 5.6 Hz, |
| | 2H), 2.79-2.71 (m, 1H), 1.88-1.79 (m, 2H), |
| | 1.68-1.60 (m, 2H), 1.58 (d, J = 7.0 Hz, 3H), |
| | 1.53-1.41 (m, 4H). LC/MS (ESI) m/z: 561 |
| | (M + H)⁺. RT (Method A): 1.82 min. |
| 450 | ¹H NMR (400 MHz, CD₃OD) δ 8.70 (s, 1H), 8.09 |
| | (d, J = 5.9 Hz, 1H), 8.03-7.98 (m, 2H), 7.85 (s, |
| | 1H), 7.51-7.47 (m, 3H), 7.39 (d, J = 5.8 Hz, |
| | 1H), 7.11 (s, 1H), 6.56 (s, 1H), 5.90 (s, 1H), 4.76 |
| | (s, 2H), 4.62-4.59 (m, 1H), 4.58 (s, 2H), 2.19- |
| | 2.15 (m, 2H), 2.05-2.01 (m, 2H), 1.64 (d, J = |
| | 6.7 Hz, 3H), 1.61-1.57 (m, 2H), 1.32-1.28 (m, |
| | 2H). LC/MS (ESI) m/z: 550 (M + H)⁺. RT (Method |
| | A): 1.38 min. |
| 476 | ¹H NMR (400 MHz, DMSO-d₆) δ 11.52 (s, 1H), |
| | 8.85-8.81 (m, 1H), 8.79 (s, 1H), 8.15 (s, 1H), |
| | 8.14 (s, 1H), 8.07 (s, 1H), 7.97-7.94 (m, 2H), |
| | 7.54-7.51 (m, 3H), 7.39 (d, J = 5.7 Hz, 1H), |
| | 7.14 (s, 1H), 6.49 (s, 1H), 5.11 (d, J = 8.1 Hz, |
| | 1H), 4.83-4.75 (m, 2H), 4.57-4.52 (m, 1H), |
| | 4.48 (d, J = 5.5 Hz, 2H), 3.03-2.99 (m, 1H), |
| | 1.84-1.75 (m, 4H), 1.67-1.60 (m, 2H), 1.52 |
| | (d, J = 6.6 Hz, 3H), 1.52-1.48 (m, 2H). LC/MS |
| | (ESI) m/z: 538 (M + H)⁺. RT (method A): 1.66 |
| | min. |

^{a}Step 1 was performed in the presence of Pd(PPh₃)₄ and Na₂CO₃ in a mixture of DME and water.

### Scheme 77. Synthesis of N-((1H-pyrrolo[3,2-c]pyridine-2-yl)methyl)-2-(5-(((9,9-difluoro-9H-fluoren-3-yl)methyl)amino)-6-oxo-2-(4-(pentafluoro-λ⁶-sulfaneyl)phenyl)pyrimidin-1(6H)-yl)acetamide (Compound 255)

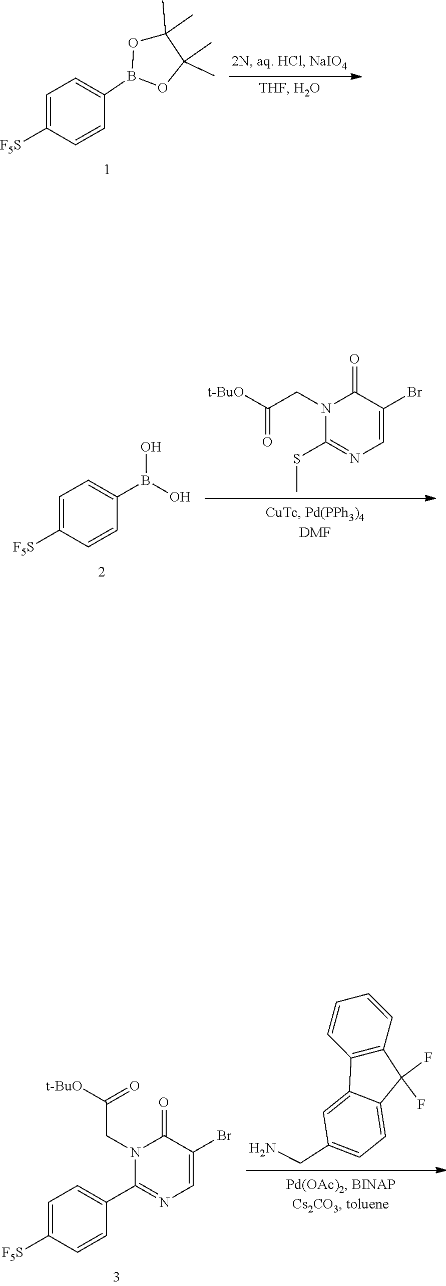

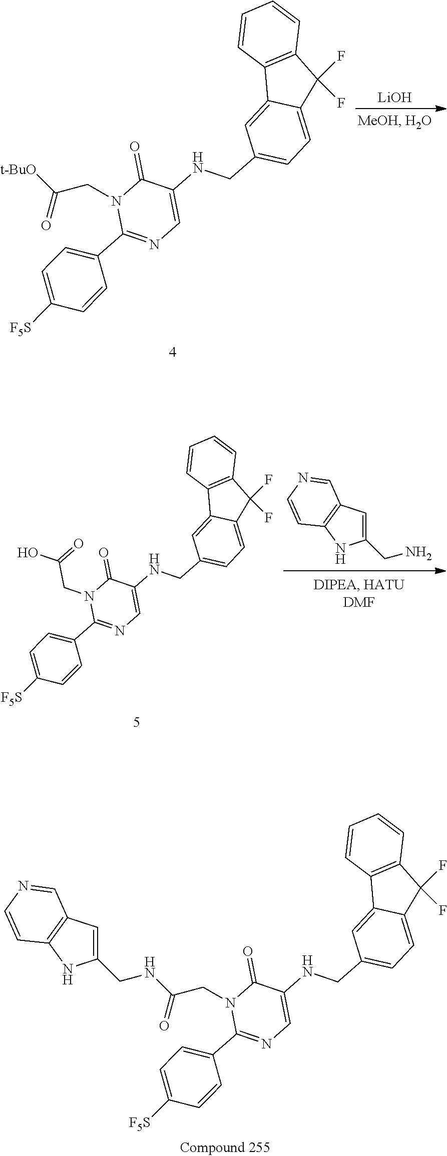

### Step 1: (4-(Pentafluoro-λ⁶-sulfaneyl)phenyl)boronic acid (2)

To a solution of 4,4,5,5-tetramethyl-2-(4-(pentafluoro-λ⁶-sulfaneyl)phenyl)-1,3,2-dioxaborolane (293 mg, 89 mmol) in THF/water (5 mL, v/v=4/1) was added NaIO₄ (570 mg, 2.66 mmol) and 1N aq. HCl (0.7 mL) and the mixture was stirred at room temperature overnight. The mixture was diluted with EtOAc, washed with water and brine, dried over anhydrous Na₂SO₄, filtered, and concentrated under reduced pressure to dryness to give the title compound (160 mg, yield 72.7%) as a white solid, which was used directly in the next step without further purification. ¹H NMR (400 MHz, CDCl₃) δ 7.83 (d, J=7.6 Hz, 2H), 7.60-7.55 (m, 2H), 6.96 (s, 2H).

### Step 2: Tert-butyl 2-(5-bromo-6-oxo-2-(4-(pentafluoro-λ⁶-sulfaneyl)phenyl) pyrimidin-1(6H)-yl)acetate (3)

To a mixture of (4-(pentafluoro-λ⁶-sulfaneyl)phenyl)boronic acid (163 mg, 0.66 mmol) and tert-butyl 2-(5-bromo-2-(methylthio)-6-oxopyrimidin-1(6H)-yl)acetate (110 mg, 0.33 mmol) in DMF (3 mL) was added CuTC (138 mg, 0.72 mmol) and Pd(PPh₃)₄ (38 mg, 0.03 mmol) under N₂ atmosphere. The mixture was degassed under N₂ atmosphere for three times and stirred at 80° C. overnight in a sealed tube. The reaction mixture was diluted with EtOAc and filtered. The filtrate was washed with saturated aq.NaHCO₃ solution and brine, dried over anhydrous Na₂SO₄, filtered, and concentrated under reduced pressure to dryness. The residue was purified by prep-TLC (PE:EtOAc=5:1) to give the title compound (69 mg, yield 42.9%) as a colorless oil. LC/MS (ESI) m/z: 491 (M+H)⁺.

### Step 3: Tert-butyl 2-(5-(((9,9-difluoro-9H-fluoren-3-yl)methyl)amino)-6-oxo-2-(4-(pentafluoro-λ⁶-sulfaneyl)phenyl)pyrimidin-1(6H)-yl)acetate (4)

To a mixture of tert-butyl 2-(5-bromo-6-oxo-2-(4-(pentafluoro-λ⁶-sulfaneyl)phenyl) pyrimidin-1(6H)-yl)acetate (69 mg, 0.14 mmol) and (9,9-difluoro-9H-fluoren-3-yl)methanamine (65 mg, 0.28 mmol) in toluene (2 mL) was added Cs₂CO₃ (92 mg, 0.28 mmol), Pd(OAc)₂ (3.0 mg, 0.01 mmol) and BINAP (17.5 mg, 0.03 mmol) under N₂ atmosphere, the reaction mixture was degassed under N₂ atmosphere for three times and stirred at 120° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (55 mg, yield 60.9%) as a yellow oil. LC/MS (ESI) m/z: 642 (M+H)⁺.

### Step 4: 2-(5-(((9,9-Difluoro-9H-fluoren-3-yl)methyl)amino)-6-oxo-2-(4-(pentafluoro-λ⁶-sulfaneyl)phenyl)pyrimidin-1(6H)-yl)acetic acid (5)

To a solution of tert-butyl 2-(5-(((9,9-difluoro-9H-fluoren-3-yl)methyl)amino)-6-oxo-2-(4-(pentafluoro-λ⁶-sulfaneyl)phenyl)pyrimidin-1(6H)-yl)acetate (23 mg, 0.04 mmol) in MeOH/water (1.5 mL, v/v=2/1) was added LiOH (4 mg, 0.17 mmol) and the mixture was stirred at 60° C. for 2 hours. The mixture was acidified with 1 N aq. HCl to pH˜5 and extracted with EtOAc twice. The combined organic layers were washed with brine, dried over anhydrous Na₂SO₄, filtered, and concentrated under reduced pressure to dryness to give the title compound (20 mg, yield 95.2%) as a yellow solid, which was used directly in the next step without further purification. LC/MS (ESI) m/z: 586 (M+H)⁺.

### Step 5: N-((1H-pyrrolo[3,2-c]pyridine-2-yl)methyl)-2-(5-(((9,9-difluoro-9H-fluoren-3-yl)methyl)amino)-6-oxo-2-(4-(pentafluoro-λ⁶-sulfaneyl)phenyl)pyrimidin-1(6H)-yl)acetamide (Compound 255)

To a mixture of 2-(5-(((9,9-difluoro-9H-fluoren-3-yl)methyl)amino)-6-oxo-2-(4-(pentafluoro-λ⁶-sulfaneyl)phenyl)pyrimidin-1(6H)-yl)acetic acid (20 mg, 0.03 mmol) and (1H-pyrrolo[3,2-c]pyridine-2-yl)methanamine hydrochloride (11 mg, 0.06 mmol) in DMF (1 mL) was added DIPEA (22 mg, 0.17 mmol) and HATU (20 mg, 0.05 mmol) under N₂ atmosphere and the reaction mixture was stirred at room temperature for 1 hour. The mixture was diluted with EtOAc, washed with saturated aq.NH₄Cl solution and brine, dried over anhydrous Na₂SO₄, filtered, and concentrated under reduced pressure to dryness. The residue was purified by prep-TLC (DCM:MeOH=9:1) and further purified by prep-HPLC to give Compound 255 (5.7 mg, yield 23.4%) as a white solid. ¹H NMR (400 MHz, CD₃OD) δ 8.73 (s, 1H), 8.13 (d, J=5.9 Hz, 1H), 7.77 (d, J=8.9 Hz, 3H), 7.68 (d, J=7.7 Hz, 1H), 7.64 (d, J=8.4 Hz, 2H), 7.58 (t, J=6.9 Hz, 2H), 7.50 (t, J=7.4 Hz, 1H), 7.45-7.41 (m, 2H), 7.38 (t, J=7.5 Hz, 1H), 7.07 (s, 1H), 6.54 (s, 1H), 4.62 (s, 2H), 4.57 (s, 2H), 4.54 (s, 2H). LC/MS (ESI) m/z: 715 (M+H)⁺. RT (Method A): 2.05 min.

The following compounds were prepared based on Steps 3-5 in Scheme 77:

| # | Reactant A | Reactant B | Reactant C |
|---|---|---|---|
| 138^{a} | 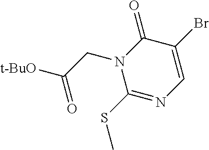 [SMILES: CSc1ncc(Br)c(=O)n1CC(=O)OC(C)(C)C] | 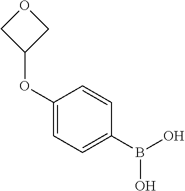 [SMILES: OB(O)c1ccc(OC2COC2)cc1] | 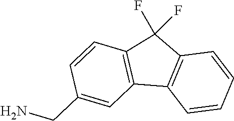 [SMILES: NCc1ccc2c(c1)-c1ccccc1C2(F)F] |
| 330 | 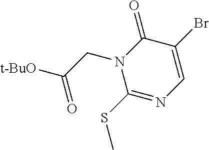 [SMILES: CSc1ncc(Br)c(=O)n1CC(=O)OC(C)(C)C] | 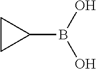 | 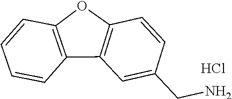 [SMILES: NCc1ccc2oc3ccccc3c2c1 ; Cl] |
| 391 | 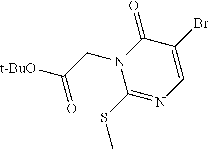 [SMILES: CSc1ncc(Br)c(=O)n1CC(=O)OC(C)(C)C] | 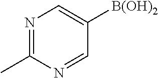 | 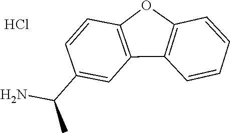 [SMILES: Cl ; C[C@@H](N)c1ccc2oc3ccccc3c2c1] |
| 434^{b,c} | 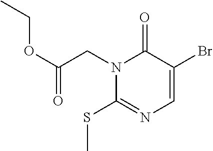 [SMILES: CCOC(=O)Cn1c(SC)ncc(Br)c1=O] | 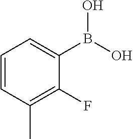 | 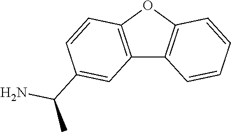 [SMILES: C[C@@H](N)c1ccc2oc3ccccc3c2c1] |
| 435^{c,d} | 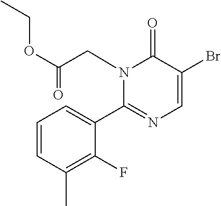 [SMILES: CCOC(=O)Cn1c(-c2cccc(C)c2F)ncc(Br)c1=O] | | 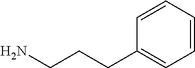 |
| 436^{b,c} | 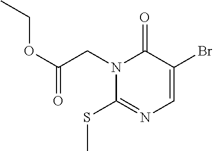 [SMILES: CCOC(=O)Cn1c(SC)ncc(Br)c1=O] | 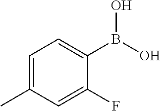 | 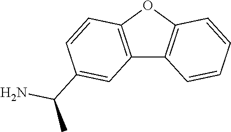 [SMILES: C[C@@H](N)c1ccc2oc3ccccc3c2c1] |
| 437^{c,d} | 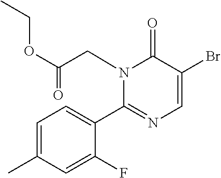 [SMILES: CCOC(=O)Cn1c(-c2ccc(C)cc2F)ncc(Br)c1=O] | | 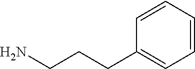 |
| 438^{b,c} | 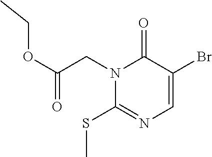 [SMILES: CCOC(=O)Cn1c(SC)ncc(Br)c1=O] | 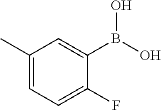 | 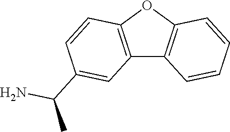 [SMILES: C[C@@H](N)c1ccc2oc3ccccc3c2c1] |
| 439^{c,d} | 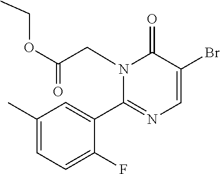 [SMILES: CCOC(=O)Cn1c(-c2cc(C)ccc2F)ncc(Br)c1=O] | | 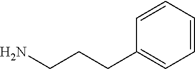 |
| 483^{b,c} | 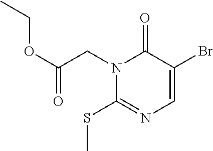 [SMILES: CCOC(=O)Cn1c(SC)ncc(Br)c1=O] | 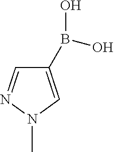 | 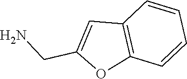 [SMILES: NCc1cc2ccccc2o1] |

| # | Reactant D | Characterization Data |
|---|---|---|
| 138^{a} | 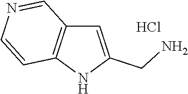 [SMILES: NCc1cc2cnccc2[nH]1 ; Cl] | ¹H NMR (400 MHz, DMSO-d₆) δ 11.30 (s, 1H), 8.76 (s, 1H), 8.68 (t, J = 5.6 Hz, 1H), 8.13 (d, J = 5.7 Hz, 1H), 7.87 (s, 1H), 7.80 (d, J = 7.5 Hz, 1H), 7.68 (t, J = 8.0 Hz, 2H), 7.58 (t, J = 7.5 Hz, 1H), 7.43 (t, J = 7.8 Hz, 2H), 7.36 (dd, J = 6.9, 5.1 Hz, 3H), 7.04 (s, 1H), |
| | | 6.73 (d, J = 8.7 Hz, 2H), 6.33 (m, 2H), 5.21 |
| | | (p, J = 5.4 Hz, 1H), 4.88 (t, J = 6.7 Hz, 2H), |
| | | 4.51 (t, J = 6.0 Hz, 4H), 4.43 (d, J = 5.6 Hz, |
| | | 4H). LC/MS (ESI) m/z: 661 (M + H)⁺. RT |
| | | (Method A): 1.67 min. |
| 330 | 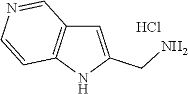 [SMILES: NCc1cc2cnccc2[nH]1 ; Cl] | ¹H NMR (400 MHz, DMSO-d₆) δ 11.36 (s, 1H), 8.84 (s, 1H), 8.73 (s, 1H), 8.12 (s, 3H), 7.70-7.62 (m, 2H), 7.51 (t, J = 8.9 Hz, 2H), 7.38 (t, J = 7.2 Hz, 1H), 7.32 (d, J = 5.6 Hz, 1H), 6.87 (m, 1H), 6.44 (s, 1H), 6.04 (m, 1H), 4.94 (s, 2H), 4.48 (d, J = 4.3 Hz, 2H), 4.41 (d, |
| | | J = 5.8 Hz, 2H), 1.81 (d, J = 5.6 Hz, 1H), 0.77 |
| | | (d, J = 7.7 Hz, 4H). LC/MS (ESI) m/z: 519 |
| | | (M + H)⁺. RT (Method A): 1.54 min. |
| 391 | 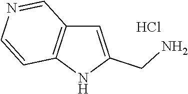 [SMILES: NCc1cc2cnccc2[nH]1 ; Cl] | ¹H NMR (400 MHz, DMSO-d₆) δ 11.36 (s, 1H), 8.83 (t, J = 5.3 Hz, 1H), 8.77-8.69 (m, 3H), 8.23 (s, 1H), 8.15-8.07 (m, 2H), 7.72- 7.60 (m, 3H), 7.55-7.48 (m, 1H), 7.44-7.37 (m, 1H), 7.32 (d, J = 5.6 Hz, 1H), 7.05 (s, 1H), 6.34 (s, 1H), 6.12 (d, J = 7.2 Hz, 1H), 4.73- 4.52 (m, 3H), 4.42 (d, J = 5.4 Hz, 2H), 2.61 |
| | | (s, 3H), 1.62 (d, J = 6.7 Hz, 3H). LC/MS (ESI) |
| | | m/z: 585 (M + H)⁺. RT (Method A): 1.51 min. |
| 434^{b,c} | 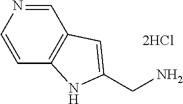 [SMILES: NCc1cc2cnccc2[nH]1 ; "2HCl"] | ¹H NMR (400 MHz, DMSO-d₆) δ 11.25 (1H, s), 8.74 (1H, s), 8.57 (1H, t, J = 5.6 Hz), 8.22 (1H, s), 8.16 (1H, m), 8.10 (1H, d, J = 7.4 Hz), 7.67 (2H, m), 7.63 (1H, m), 7.51 (1H, td, J = 7.3, 1.4 Hz), 7.31-7.39 (3H, m), 7.12 (1H, m), 7.01 (2H, m), 6.24 (1H, s), 5.99 (1H, d, J = 7.1 Hz), 4.83 (1H, m), 4.67 (1H, q, J = 6.9 |
| | | Hz), 4.33 (2H, d, J = 5.5 Hz), 4.17 (1H, m), |
| | | 2.20 (3H, s) and 1.61 (3H, d, J = 6.8 Hz). |
| | | LC/MS (ESI) m/z: 601 (M + H)⁺. RT (Method |
| | | A): 1.80 min. |
| 435^{c,d} | 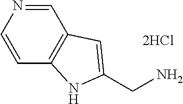 [SMILES: NCc1cc2cnccc2[nH]1 ; "2HCl"] | ¹H NMR (400 MHz, DMSO-d₆) δ 11.49 (1H, s), 8.57 (1H, t, J = 5.5 Hz), 8.13 (1H, m), 7.39 (1H, m), 7.21-7.32 (5H, m), 7.14-7.19 (2H, m), 7.06 (2H, m), 6.50 (1H, s), 6.29 (1H, s), 5.60 (1H, t, J = 5.9 Hz), 4.82 (1H, m), 4.36 (2H, d, J = 5.4 Hz), 4.16 (1H, m), 3.10 (2H, q, J = 6.7 Hz), 2.67 (2H, t, J = 7.6 Hz), 2.26 |
| | | (3H, s) and 1.89 (2H, quint, J = 7.3 Hz). |
| | | LC/MS (ESI) m/z: 525 (M + H)⁺. RT (Method |
| | | A): 1.81 min. |
| 436^{b,c} | 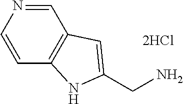 [SMILES: NCc1cc2cnccc2[nH]1 ; "2HCl"] | ¹H NMR (400 MHz, DMSO-d₆) δ 11.29 (1H, s), 8.57 (1H, m), 8.22 (1H, d, J = 1.7 Hz), 8.10 (1H, d, J = 7.4 Hz), 7.60-7.70 (4H, m), 7.51 (1H, td, J = 7.2, 1.3 Hz), 7.39 (1H, t, J = 7.8 Hz), 7.17 (1H, m), 7.09 (1H, m), 6.98 (1H, s), 6.90 (1H, m), 6.23 (1H, s), 5.98 (1H, d, J = 7.0 Hz), 4.66 (1H, q, J = 6.9 Hz), 4.45 (1H, |
| | | m), 4.34 (2H, d, J = 5.4 Hz), 4.16 (1H, m), |
| | | 2.28 (3H, s) and 1.60 (3H, d, J = 6.7 Hz). |
| | | LC/MS (ESI) m/z: 601 (M + H)⁺. RT (Method |
| | | A): 1.81 min. |
| 437^{c,d} | 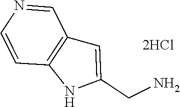 [SMILES: NCc1cc2cnccc2[nH]1 ; "2HCl"] | ¹H NMR (400 MHz, DMSO-d₆) δ 11.74 (1H, s), 8.89 (1H, s), 8.62 (1H, d, J = 5.6 Hz), 8.21 (1H, d, J = 6.0 Hz), 7.53 (1H, d, J = 6.0 Hz), 7.23-7.31 (5H, m), 7.18 (1H, m), 7.14 (1H, m), 7.08 (1H, s), 6.97 (1H, d, J = 6.9 Hz), 6.43 (1H, s), 5.58 (1H, t, J = 5.9 Hz), 4.64 (1H, m), 3.92-4.38 (3H, m), 3.09 (2H, q, J = 6.6 Hz), |
| | | 2.67 (2H, t, J = 7.5 Hz), 2.32 (3H, s) and 1.90 |
| | | (2H, m). LC/MS (ESI) m/z: 525 (M + H)⁺. RT |
| | | (Method A): 1.52 min. |
| 438^{b,c} | 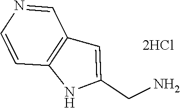 [SMILES: NCc1cc2cnccc2[nH]1 ; "2HCl"] | ¹H NMR (400 MHz, DMSO-d₆) δ 11.30 (1H, s), 8.72 (1H, s), 8.59 (1H, t, J = 5.6 Hz), 8.22 (1H, d, J = 2 Hz), 8.17 (1H, m), 8.11 (1H, d, J = 7.3 Hz), 7.68 (2H, m), 7.62 (1H, dd, J = 7.6, 2 Hz), 7.51 (1H, td, J = 7.3, 1 Hz), 7.39 (1H, td, J = 7.5, 1 Hz), 7.30 (2H, m), 7.17 (1H, m), 7.11 (1H, m), 6.99 (1H, s), 6.20 (1H, s), |
| | | 5.99 (1H, d, J = 3.1 Hz), 4.80 (1H, m), 4.68 |
| | | (1H, m), 4.16-4.40 (3H, m), 2.13 (3H, s) and |
| | | 1.61 (3H, d, J = 6.7 Hz). LC/MS (ESI) m/z: |
| | | 601 (M + H)⁺. RT (Method A): 1.81 min. |
| 439^{c,d} | 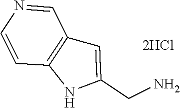 [SMILES: NCc1cc2cnccc2[nH]1 ; "2HCl"] | ¹H NMR (400 MHz, DMSO-d₆) δ 11.49 (1H, s), 8.76 (1H, s), 8.60 (1H, d, J = 5.7 Hz), 8.14 (1H, s), 7.40 (1H, m), 7.30-7.35 (1H, m), 7.22-7.29 (4H, m), 7.16-7.20 (3H, m), 7.08 (1H, s), 6.27 (1H, s), 5.61 (1H, t, J = 6.0 Hz), 4.80 (1H, m), 4.02-4.30 (3H, m), 3.11 (2H, q, J = 6.7 Hz), 2.66 (2H, t, J = 7.7 Hz), 2.19 (3H, |
| | | s) and 1.90 (2H, quint, J = 7.4 Hz). LC/MS |
| | | (ESI) m/z: 525 (M + H)⁺. RT (Method A): 1.53 |
| | | min. |
| 483^{b,c} | 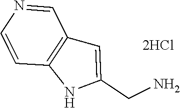 [SMILES: NCc1cc2cnccc2[nH]1 ; "2HCl"] | ¹H NMR (400 MHz, MeOD-d₄) δ 8.73 (1H, s), 8.11 (1H, m), 7.77 (1H, s), 7.72 (2H, m), 7.64 (1H, s), 7.48 (1H, d, J = 7.5 Hz), 7.43 (1H, d, J = 6.0 Hz), 7.38 (1H, d, J = 8.1 Hz), 7.12- 7.24 (2H, m), 6.68 (1H, s), 6.58 (1H, s), 4.82 (1H, m), 4.65 (2H, s), 4.79 (1H, m), 4.61 (2H, m), 4.53 (2H, s) and 3.81 (3H, s). LC/MS |
| | | (ESI) m/z: 509 (M + H)⁺. RT (Method A): 0.98 |
| | | min. |

^{a}Pd₂(dba)₂ was used in place of Pd(OAc)₂ in Step 3.
^{b}Step 2 was performed in THF.
^{c}Step 3 was performed with NaOH in THF and MeOH.
^{d}Steps 3-5 only.

### Scheme 78. Synthesis of N-[6-oxo-1-[2-oxo-2-(1H-pyrrolo[3,2-c]pyridine-2-ylmethylamino)ethyl]-2-[4-(2-pyridylmethoxy)phenyl]pyrimidin-5-yl]-4-phenyl-benzamide (Compound 225)

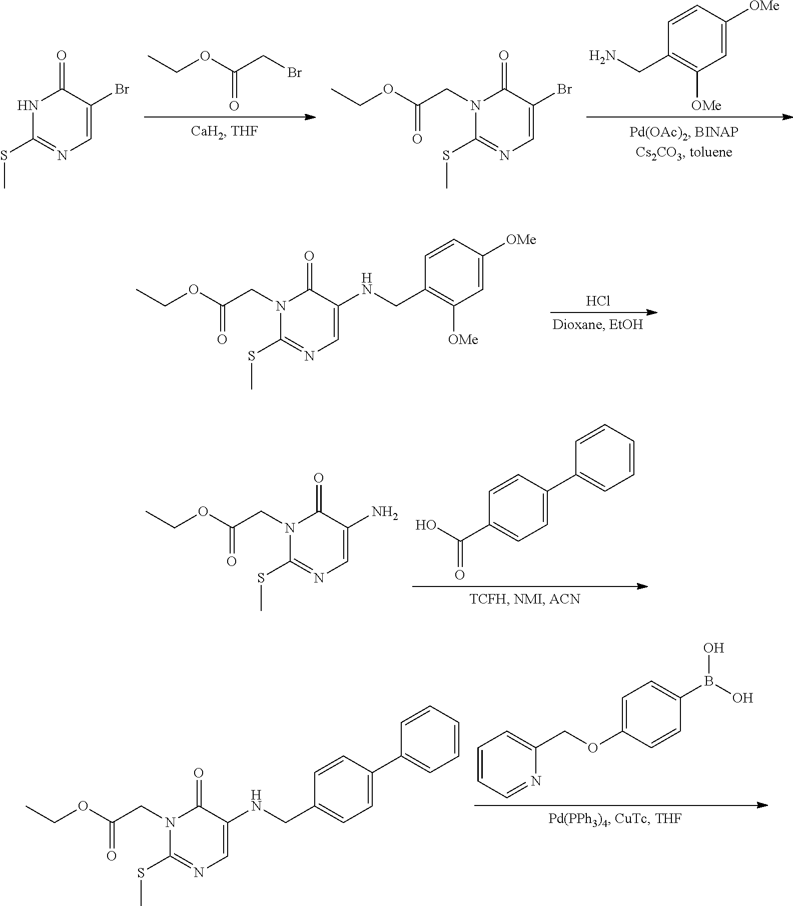

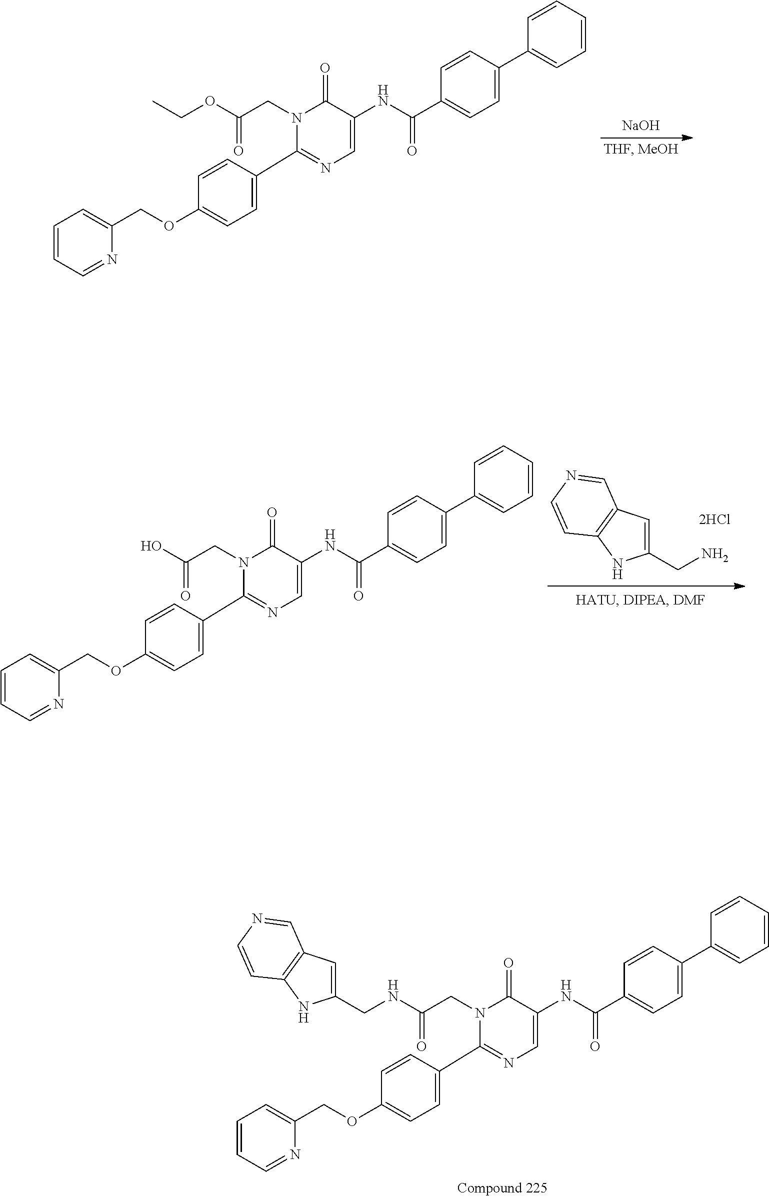

### Step 1: Ethyl 2-(5-bromo-2-methylsulfanyl-6-oxo-pyrimidin-1-yl)acetate (2)

5-bromo-2-methylsulfanyl-1H-pyrimidin-6-one 1 (3.0 g, 13.57 mmol) and ethyl-2-bromoacetate (4.55 g, 27.2 mmol) were added sequentially to a stirred ice-cold slurry of calcium hydride (1.28 g, 27.4 mmol) in dry THF (30 mL). The reaction was stirred at 0° C. for 1 hour and then allowed to warm to RT and stirred at RT for 2 hours and then warmed to 60° C. and stirred overnight at that temperature. The reaction was cooled to 0° C. and poured very slowly into a stirred ice-water mixture. The reaction was extracted with ethyl acetate (3×40 mL). The combined organic phase was washed with brine, dried over MgSO₄, filtered, and concentrated in vacuo to give the product mix as a white solid. The residue was purified by flash chromatography (silica gel, 5-50% EtOAc in hexanes) to give the title compound (2.83 g, yield 68%) as a white solid. LC/MS (ESI) m/z: 307 and 309 (M+H, for Br isotopes)⁺.

### Step 2: Ethyl 2-[5-[(2,4-dimethoxyphenyl)methylamino]-2-methylsulfanyl-6-oxo-pyrimidin-1-yl]acetate (3)

Ethyl 2-(5-bromo-2-methylsulfanyl-6-oxo-pyrimidin-1-yl)acetate 2 (100 mg, 0.326 mmol), Palladium acetate (8 mg, 10 mol %), BINAP (41 mg, 20 mol %) and cesium carbonate (319 mg, 0.98 mmol) were weighed out into a screw cap pressure vial. A solution of dimethoxyphenyl)methanamine (273 mg, 1.63 mmol) in toluene (6 mL) was added and the reaction was stirred and purged with nitrogen bubbling for 10 mins and then sealed under a dry nitrogen atmosphere. The reaction was warmed to 110° C. and stirred overnight at this temperature. The reaction was cooled to room temperature, carefully opened, and diluted with ethyl acetate (10 mL) and saturated ammonium chloride solution (10 mL) and stirred for 5 mins. The phases were separated and then the aqueous phase was further extracted with ethyl acetate (2×10 mL). The combined organic phase was washed with saturated ammonium chloride solution, water and then brine, dried over MgSO₄, filtered, and concentrated in vacuo to a black oil. The residue was purified by flash chromatography (silica gel, 5-40% EtOAc in hexanes) to give the title compound (89 mg, yield 70%) as a yellow oil. LC/MS (ESI) m/z: 394 (M+H)⁺.

### Step 3: Ethyl 2-(5-amino-2-methylsulfanyl-6-oxo-pyrimidin-1-yl)acetate (4)

To a solution of ethyl 2-[5-[(2,4-dimethoxyphenyl)methylamino]-2-methylsulfanyl-6-oxo-pyrimidin-1-yl]acetate 3 (85 mg, 0.216 mmol) in ethanol (2 mL) was added a solution of HCl (4M in dioxane: 4 mL, Xs). The reaction was stirred at room temperature for 2 hours, at which time LC/MS showed complete loss of starting material. The reaction was diluted with water (3 mL) and then carefully neutralized to pH 7-8 with sat. NaHCO₃ solution. The reaction was extracted with ethyl acetate (2×20 mL). The combined organic phase was washed with brine (2×), dried over MgSO₄, filtered, and concentrated in vacuo to give the product as a yellow viscous oil (56 mg). LC/MS (ESI) m/z: 244.15 (M+H)⁺.

### Step 4: Ethyl 2-[2-methylsulfanyl-6-oxo-5-[(4-phenylbenzoyl)amino]pyrimidin-1-yl]acetate (5)

Ethyl 2-(5-amino-2-methylsulfanyl-6-oxo-pyrimidin-1-yl)acetate 4 (75 mg, 0.31 mmol) and biphenyl-4-carboxylic acid (310 mg, 1.6 mmol) were dissolved in 5 mL acetonitrile. 1-Methylimidazole (0.25 mL, 3.1 mmol) was added followed by the addition of TCFH (440 mg, 1.5 mmol). The reaction was stirred at room temperature for 24 hours and then quenched with the addition of sat. NaHCO₃ solution (4 mL). The reaction was stirred for 10 mins and then diluted with water (5 mL) and extracted with ethyl acetate (3×15 mL). The combined organic phase was washed with brine, dried over MgSO₄, filtered, and concentrated in vacuo. The residue was purified by flash chromatography (silica gel, 10-60% EtOAc in hexanes) to give the title compound (128 mg, yield 98%) as a white solid. LC/MS (ESI) m/z: 424 (M+H)⁺.

### Step 5: Ethyl 2-[6-oxo-5-[(4-phenylbenzoyl)amino]-2-[4-(2-pyridylmethoxy)phenyl]pyrimidin-1-yl]acetate (6)

To a suspension of ethyl 2-[2-methylsulfanyl-6-oxo-5-[(4-phenylbenzoyl)amino]pyrimidin-1-yl]acetate 5 (65 mg, 0.154 mmol), [4-(2-pyridylmethoxy)phenyl]boronic acid (70 mg, 0.306 mmol) and CuTc (60 mg, 0.308 mmol) in dry THF (3 mL) was added Pd(PPh₃)₄ (17 mg, 10 mol %). The reaction was stirred at room temperature and was deoxygenated with N₂ bubbling through the solution for 5 mins. The reaction was then sealed under a dry N₂ atmosphere and warmed to 50° C. and stirred overnight at 50° C. The reaction was cooled to room temperature, diluted with ethyl acetate (30 mL) and then washed with sat NH₄Cl solution (3×), brine and dried over MgSO₄, filtered and concentrated in vacuo. The residue was purified by flash chromatography (silica gel, 20-80% EtOAc in hexanes) to give the title compound 6 (48 mg, yield 56%) as a white solid. LC/MS (ESI) m/z: 561 (M+H)⁺.

### Step 6: 2-[6-Oxo-5-[(4-phenylbenzoyl)amino]-2-[4-(2-pyridylmethoxy)phenyl]pyrimidin-1-yl]acetic acid (7)

A suspension of ethyl 2-[6-oxo-5-[(4-phenylbenzoyl)amino]-2-[4-(2-pyridylmethoxy)-phenyl]pyrimidin-1-yl]acetate 6 (48 mg, 0.086 mmol) in THF (3 mL) and methanol (0.5 mL) was treated with 1N sodium hydroxide solution (0.18 mL, 0.18 mmol) at room temperature. After 60 mins, LC/MS showed the complete consumption of starting material. The pH of the reaction was adjusted to pH 5 with 2N HCl solution, and then extracted with ethyl acetate (3×15 mL). The combined organic phase was washed with brine, dried over MgSO₄, filtered, and concentrated in vacuo to give the product as a white powder (35 mg, 78%), which was used without further purification. LC/MS (ESI) m/z: 533 (M+H)⁺.

### Step 7: N-[6-Oxo-1-[2-oxo-2-(1H-pyrrolo[3,2-c]pyridine-2-ylmethylamino)ethyl]-2-[4-(2-pyridylmethoxy)phenyl]pyrimidin-5-yl]-4-phenyl-benzamide (Compound 225)

To a solution of the starting material acid 7 (35 mg, 0.066 mmoL) and 1H-pyrrolo[3,2-c]pyridine-2-ylmethanamine dihydrochloride (18 mg, 0.082 mmol) in DMF (1.5 mL) at room temperature was added HATU (39 mg, 0.1 mmol) and Hunig's base (0.06 mL, 0.3 mmol). The reaction was sealed under a dry N₂ atmosphere and stirred for 60 mins at 0° C., by which time LC/MS showed the complete consumption of starting acid. The reaction was poured into water and extracted with ethyl acetate (3×10 mL). The combined organic phase was washed with brine, dried over MgSO₄, filtered, and concentrated in vacuo. The residue was purified by prep-HPLC to give Compound 225 (12 mg, yield 28%) as a white solid. ¹H NMR (400 MHz, DMSO-d₆) δ 11.37 (1H, s), 9.55 (1H, s), 8.79-8.85 (2H, m), 8.76 (1H, s), 8.61 (1H, d, J=4.9 Hz), 8.22 (1H, s), 8.13 (1H, d, J=5.7 Hz), 8.08 (2H, d, J=8.3 Hz), 7.84-7.89 (3H, m), 7.78 (2H, d, J=7.8 Hz), 7.50-7.57 (4H, m), 7.47 (1H, m), 7.38 (1H, dd, J=7.5, 5.0 Hz), 7.34 (1H, d, J=5.7 Hz), 7.11 (2H, d, J=8.7 Hz), 6.34 (1H, s), 5.22 (2H, s), 4.64 (2H, s) and 4.46 (2H, d, J=5.5 Hz). LC/MS (ESI) m/z: 662 (M+H)⁺. RT (Method A): 1.53 min.

### Scheme 79. Synthesis of 2-[5-(Dibenzofuran-2-ylmethylamino)-6-oxo-pyrimidin-1-yl]-N-(1H-pyrrolo[3,2-c]pyridine-2-ylmethyl)acetamide (Compound 246)

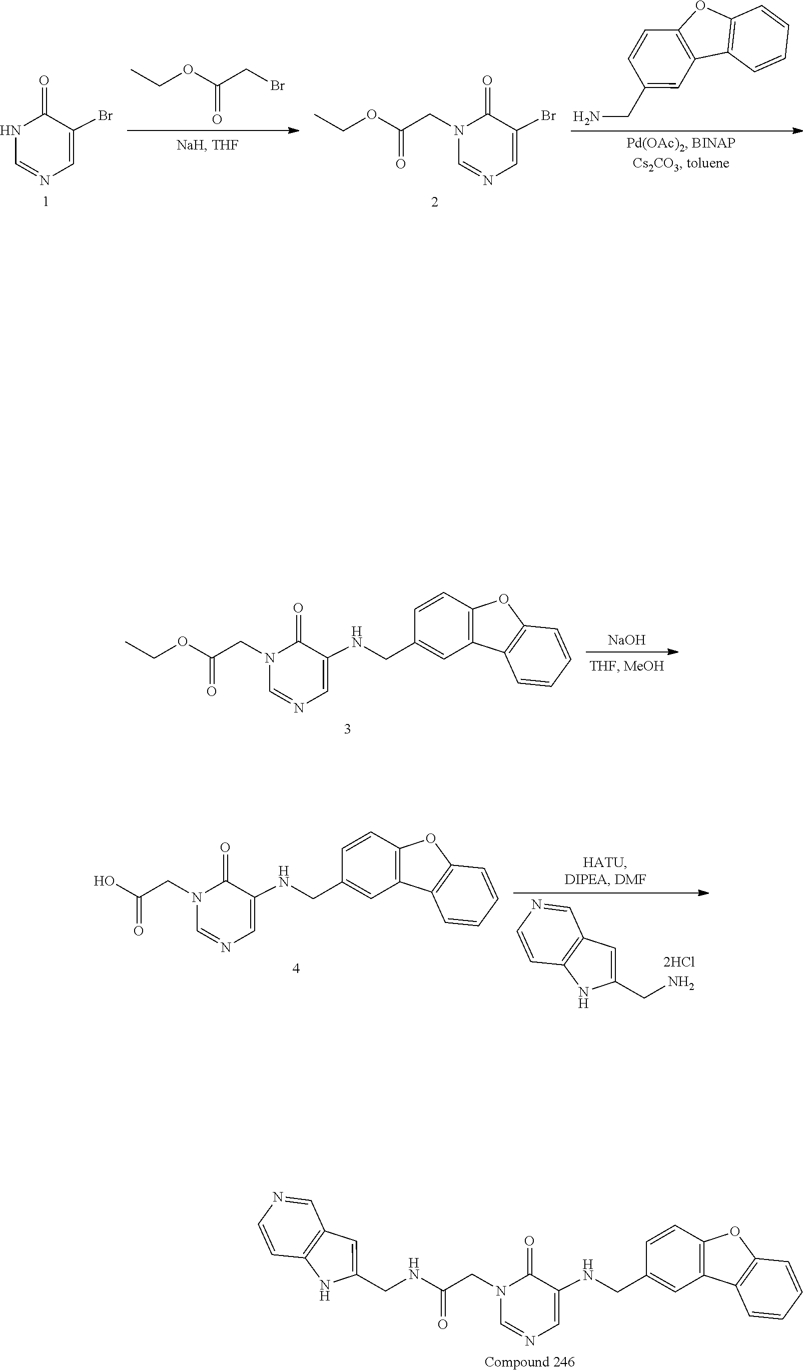

### Step 1: Ethyl 2-(5-bromo-6-oxo-pyrimidin-1-yl)acetate (2)

To an ice-cold suspension of sodium hydride (60 mass %) in oil (230 mg, 5.75 mmol, 2.0 equiv.) in THF (10 mL) was added 5-bromo-2-methylsulfanyl-1H-pyrimidin-6-one (500 mg, 2.86 mmol, 1.0 equiv.). The reaction was stirred at 0° C. for 30 minutes and then ethyl 2-bromoacetate (950 mg, 5.7 mmol, 2.0 equiv.) was added and the reaction was allowed to warm to room temperature and then stirred overnight at room temperature, at which time TLC and LC/MS showed complete consumption of starting material. The reaction was diluted with water (10 mL) and EtOAc (10 mL). The two layers were separated, and the aqueous layer was extracted with EtOAc (2×10 mL). The combined organic extracts were washed with brine, dried over MgSO₄, filtered, and concentrated to give a yellow solid. The solid was triturated with 20% ethyl acetate in hexanes and filtered and suction dried to give the product as a white powdery solid (683 mg, 91%). LC/MS (ESI) m/z: 261/263 (M+H for Br isotopes)⁺.

### Step 2: Ethyl 2-[5-(dibenzofuran-2-ylmethylamino)-6-oxo-pyrimidin-1-yl]acetate (3)

Ethyl 2-(5-bromo-6-oxo-pyrimidin-1-yl)acetate 2 (100 mg, 0.38 mmol), Palladium acetate (9 mg, 10 mol %), BINAP (48 mg, 20 mol %) and cesium carbonate (250 mg, 0.76 mmol) were weighed out into a screw cap pressure vial. Dibenzofuran-2-ylmethanamine (151 mg, 0.76 mmol) in toluene (2 mL) was added and then the reaction was stirred and purged with nitrogen bubbling for 10 mins and then sealed under a dry nitrogen atmosphere. The reaction was warmed to 110° C. and stirred overnight at this temperature. The reaction was cooled to RT, carefully opened, and then diluted with ethyl acetate (10 mL) and sat. NH₄Cl solution (5 mL) and water (5 mL) and stirred for 5 mins. The phases were separated and then the aqueous phase was further extracted with ethyl acetate (2×10 mL). The combined organic phase was washed with sat. NH₄Cl, water and then brine, dried over MgSO₄, filtered, and concentrated in vacuo to a dark orange oil. The residue was purified by flash chromatography (silica gel, 20-100% EtOAc in hexanes) to give the title compound (106 mg, yield 73%) as a pale-yellow solid. LC/MS (ESI) m/z: 378 (M+H)⁺.

### Step 3: 2-[5-(Dibenzofuran-2-ylmethylamino)-6-oxo-pyrimidin-1-yl]acetic acid (4)

1N NaOH solution (0.4 mL, 0.4 mmol) was added to a stirred solution of ethyl 2-[5-(dibenzofuran-2-ylmethylamino)-6-oxo-pyrimidin-1-yl]acetate (50 mg, 0.133 mmol) in THF (3 mL) and MeOH (0.5 mL). The reaction was stirred at room temperature for 30 mins, at which time LC/MS showed complete consumption of the ethyl ester. The pH was adjusted to pH 4-5 with 2N HCl, and then the reaction was diluted with brine (5 mL) and extracted with ethyl acetate (3×10 mL). The combined org phase was washed with brine, dried over MgSO₄, filtered, and concentrated in vacuo to give the product as an off-white solid (38 mg, 81%) which was used in the next step without the need for further purification. LC/MS (ESI) m/z: 350.21 (M+H)⁺.

### Step 4: 2-[5-(Dibenzofuran-2-ylmethylamino)-6-oxo-pyrimidin-1-yl]-N-(1H-pyrrolo[3,2-c]pyridine-2-ylmethyl)acetamide (Compound 246)

To a solution of 2-[5-(dibenzofuran-2-ylmethylamino)-6-oxo-pyrimidin-1-yl]acetic acid 4 (25 mg, 0.072 mmoL) and 1H-pyrrolo[3,2-c]pyridine-2-ylmethanamine dihydrochloride (19 mg, 0.086 mmol) in DMF (1 mL) at room temperature was added HATU (41 mg, 0.108 mmol) and Hunig's base (0.062 mL, 0.36 mmol). The reaction was sealed under a dry N₂ atmosphere and stirred for 45 mins, by which time LC/MS showed the complete consumption of starting acid. The reaction was poured into water and extracted with ethyl acetate (3×10 mL). The combined organic phase was washed with brine, dried over MgSO₄, filtered, and concentrated in vacuo. The residue was purified by prep-HPLC to give Compound 246 (12 mg, yield 35%) as a white solid. ¹H NMR (400 MHz, DMSO-d₆) δ 11.39 (1H, s), 8.85 (1H, t, J=5.7 Hz), 8.74 (1H, s), 8.22 (1H, s), 8.08-8.14 (2H, m), 7.71 (1Hs), 7.68 (1H, d, J=8.2 Hz), 7.65 (1H, d, J=8.5 Hz), 7.48-7.54 (2H, m), 7.39 (1H, t, J=7.0), 7.33 (1H, d, J=5.6 Hz), 6.98 (1H, s), 6.44 (1H, s), 6.18 (1H, t, J=6.3 Hz), 4.65 (2H, s), 4.48 (2H, d, J=5.5 Hz) and 4.44 (2H, d, J=6.1 Hz). LC/MS (ESI) m/z: 479.24 (M+H)⁺. RT (Method A): 1.35 min.

Compound 247 was prepared based on Scheme 79:

| # | Reactant A | Reactant B | Reactant C | Reactant D |
|---|---|---|---|---|
| 247 | 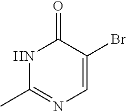 | 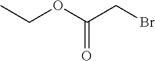 | 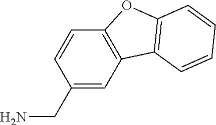 [SMILES: NCc1ccc2oc3ccccc3c2c1] | 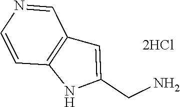 [SMILES: NCc1cc2cnccc2[nH]1 ; "2HCl"] |
| | | | # | Characterization Data |
| | | | 247 | ¹H NMR (400 MHz, DMSO-d₆) δ |
| | | | | 11.50 (1H, s), 8.85 (1H, t, J = 5.7 |
| | | | | Hz), 8.78 (1H, s), 8.08-8.16 (3H, m), |
| | | | | 7.68 (1H, d, J = 8.2 Hz), 7.64 (1H, |
| | | | | d, J = 8.5 Hz), 7.51 (2H, m), 7.39 |
| | | | | (2H, m), 6.90 (1H, s), 6.48 (1H, s), |
| | | | | 5.91 (1H, t, J = 6.3 Hz), 4.76 (2H, |
| | | | | s), 4.49 (2H, d, J = 5.5 Hz), 4.28 |
| | | | | (2H, d, J = 6.1 Hz) and 2.26 (3H, s). |
| | | | | LC/MS (ESI) m/z: 493.25 (M + H)⁺. |
| | | | | RT (Method A): 1.01 min. |

### Scheme 80. Synthesis of 2-[5-[[(1R)-1-dibenzofuran-2-ylethyl]amino]-2-(o-tolyl)-6-oxo-pyrimidin-1-yl]-N-(1H-pyrrolo[3,2-c]pyridine-2-ylmethyl)acetamide (Compound 356)

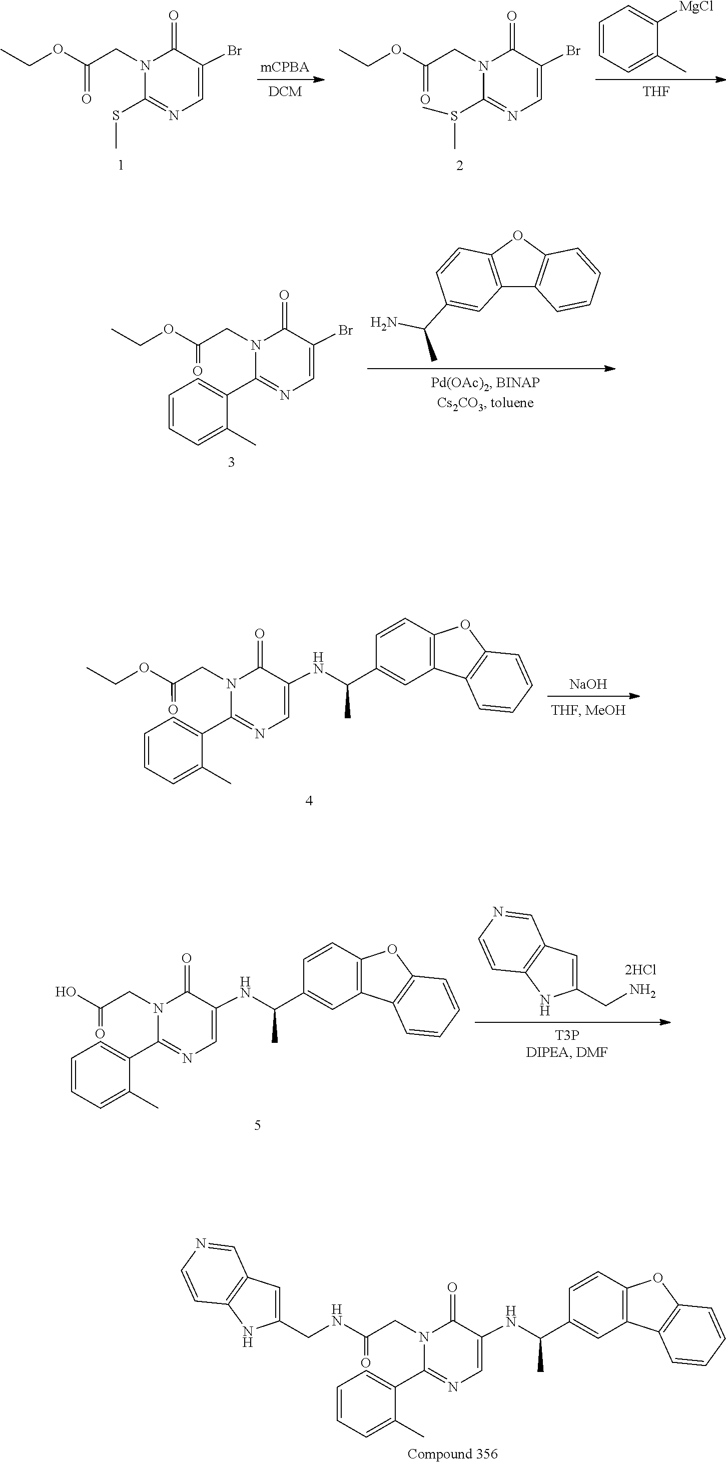

### Step 1: Ethyl 2-(5-bromo-2-methylsulfinyl-6-oxo-pyrimidin-1-yl)acetate 2

A solution of ethyl 2-(5-bromo-2-methylsulfanyl-6-oxo-pyrimidin-1-yl)acetate, 1 (1 g, 3.26 mmol) in dry DCM (20 mL) was cooled to 0° C. and then m-CPBA (1.24 g, 7.19 mmol) was added as a solid in 3 portions over 20 mins. The reaction was stirred at 0° C. for 1 hour at which time TLC analysis (50% EA in hexanes) showed the complete consumption of starting material and LC/MS analysis confirmed the presence of sulfoxide product. The reaction was quenched by the careful addition of sat. sodium thiosulfate solution (10 mL) and brine (10 mL). The phases were separated, and the aqueous phase was further extracted with DCM (2×10 mL). The combined DCM phase was washed with brine, dried over MgSO₄, filtered, and concentrated in vacuo. The residue was purified by flash chromatography (silica gel, 30->80% EtOAc in hexanes) to give the title compound (822 mg, yield 78%) as a colorless oil. LC/MS (ESI) m/z: 324 (M+H)⁺.

### Step 2: Ethyl 2-[5-bromo-2-(o-tolyl)-6-oxo-pyrimidin-1-yl]acetate 3

A solution of o-tolylmagnesium chloride (1 M in THF: 0.77 mL, 0.77 mmol) was added dropwise to a cold (0° C.) solution of the sulfoxide, ethyl 2-(5-bromo-2-methylsulfinyl-6-oxo-pyrimidin-1-yl)acetate 2, (200 mg, 0.62 mmol) in dry THF (4 mL). The reaction was stirred for a further 1 hour at 0° C., at which time TLC and LC/MS showed the complete consumption of starting sulfoxide. The reaction was quenched with the careful addition of sat. NH₄Cl solution and brine, and then extracted with ethyl acetate (3×10 mL). The combined organic phase was washed with brine, dried over MgSO₄, filtered, and concentrated in vacuo. The residue was purified by flash chromatography (silica gel, 0->50% EtOAc in hexanes) to give the title compound 3 (58 mg, yield 27%) as a colorless oil. LC/MS (ESI) m/z: 351.12/353.15 (M+H for Br isotopes)⁺.

### Step 3: Ethyl 2-[5-[[(1R)-1-dibenzofuran-2-ylethyl]amino]-2-(o-tolyl)-6-oxo-pyrimidin-1-yl]acetate 4

Ethyl 2-[5-bromo-2-(o-tolyl)-6-oxo-pyrimidin-1-yl]acetate 3 (58 mg, 0.17 mmol), Palladium acetate (3.7 mg, 10 mol %), BINAP (21 mg, 20 mol %) and cesium carbonate (162 mg, 0.49 mmol) were weighed out into a screw cap pressure vial. 1R-Dibenzofuran-2-ylethanamine (53 mg, 0.25 mmol) in toluene (3 mL) was added and then the reaction was stirred and purged with nitrogen bubbling for 10 mins and then sealed under a dry nitrogen atmosphere. The reaction was warmed to 110° C. and stirred overnight at this temperature. The reaction was cooled to room temperature, carefully opened, and then diluted with ethyl acetate (20 mL) and sat. NH₄Cl solution (10 mL) and water (10 mL) and stirred for 5 mins. The phases were separated and then the aqueous phase was further extracted with ethyl acetate (2×10 mL). The combined organic phase was washed with sat. NH₄Cl, water and brine, dried over MgSO₄, filtered, and concentrated in vacuo to a dark orange oil. The residue was purified by flash chromatography (silica gel, 0->40% EtOAc in hexanes) to give the title compound (61 mg, yield 77%) as a colorless oil. LC/MS (ESI) m/z: 482.12 (M+H)⁺.

### Step 4: 2-[5-[[(1R)-1-Dibenzofuran-2-ylethyl]amino]-2-(o-tolyl)-6-oxo-pyrimidin-1-yl]acetic acid 5

1N NaOH (0.4 mL, 0.4 mmol) was added dropwise to a stirred solution of the ethyl ester, ethyl 2-[5-[[(1R)-1-dibenzofuran-2-ylethyl]amino]-2-(o-tolyl)-6-oxo-pyrimidin-1-yl]acetate 5, (61 mg, 0.13 mmol in THF (5 mL) and methanol (1 mL). The reaction was stirred at room temperature for 60 mins, at which time LC/MS showed the complete consumption of the starting material ester. The pH of the reaction was adjusted to pH 4-5 with 1N HCl, and then diluted with brine (10 mL). The reaction was extracted with ethyl acetate (3×20 mL). The combined organic phase was washed with brine, dried over MgSO₄, filtered, and concentrated in vacuo to give the product as a pale-yellow solid (55 mg, 96%), which was used without further purification. LC/MS (ESI) m/z: 454.11 (M+H)⁺.

### Step 5: 2-[5-[[(1R)-1-Dibenzofuran-2-ylethyl]amino]-2-(o-tolyl)-6-oxo-pyrimidin-1-yl]-N-(1H-pyrrolo[3,2-c]pyridine-2-ylmethyl)acetamid (Compound 356)

Hunig's base (0.05 mL, 0.3 mmol) was added to a cold (0° C.) stirred solution of the acid 5 (10 mg, 0.022 mmol) and 1H-pyrrolo[3,2-c]pyridine-2-ylmethanamine dihydrochloride (6 mg, 0.027 mmol) in dry DMF (1 mL). T₃P (50% solution in ethyl acetate: 0.05 mL, 0.08 mmol) was added and the reaction was stirred at 0° C. for 20 mins, at which time LC/MS showed the complete consumption of starting acid. The reaction was quenched by the addition of 5 mL sat NaHCO₃ solution and water (5 mL), and then extracted with ethyl acetate (3×10 mL). The combined organic phase was washed with sat NaHCO₃ solution, water, brine, dried over MgSO₄, filtered, and concentrated in vacuo. The residue was purified by flash chromatography (silica gel, 0->30% MeOH in DCM) to give Compound 356 (7 mg, yield 54%) as a white solid. ¹H NMR (400 MHz, DMSO-d₆) δ 12.08 (1H, s), 8.99 (1H, s), 8.67 (1H, d, J=5.3 Hz), 8.25 (2H, m), 8.10 (1H, m), 7.63-7.70 (3H, m), 7.51 (1H, t, J=7.6 Hz), 7.40 (1H, m), 7.30 (1H, m), 7.06-7.23 (4H, m), 7.00 (1H, s), 6.48 (1H, s), 5.89 (1H, dd, J=14.8, 6.8 Hz), 4.67 (1H, m), 4.59 (1H, m), 4.40 (2H, m), 4.15 (1H, m), 2.09 (3H, s) and 1.60 (3H, d, J=6.7 Hz). LC/MS (ESI) m/z: 583.07 (M+H)⁺. RT (Method A): 1.70 min.

### Scheme 81. Synthesis of 2-[5-(dibenzofuran-2-ylmethylamino)-2-(3-fluoroazetidin-1-yl)-6-oxo-pyrimidin-1-yl]-N-(1H-pyrrolo[3,2-c]pyridine-2-ylmethyl)acetamide (Compound 270)

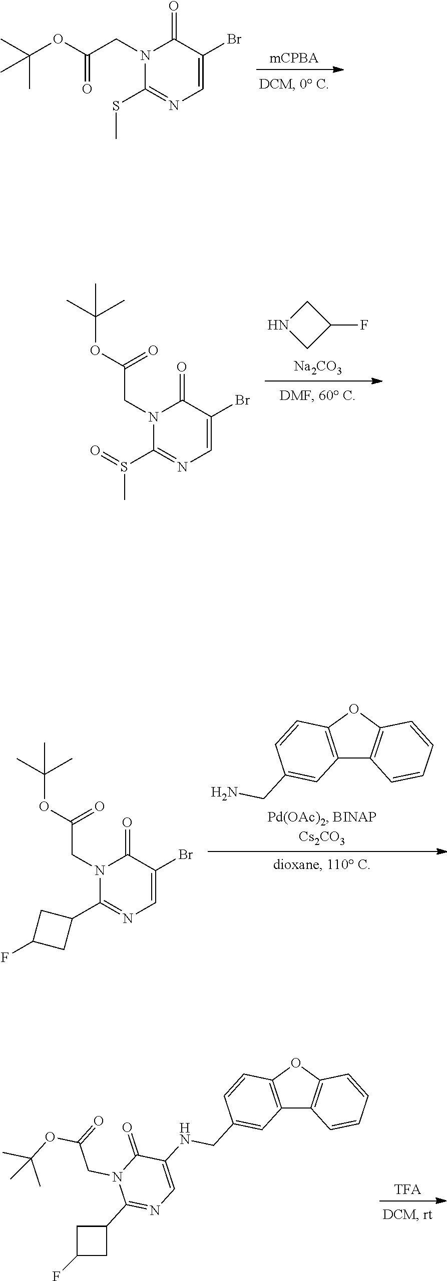

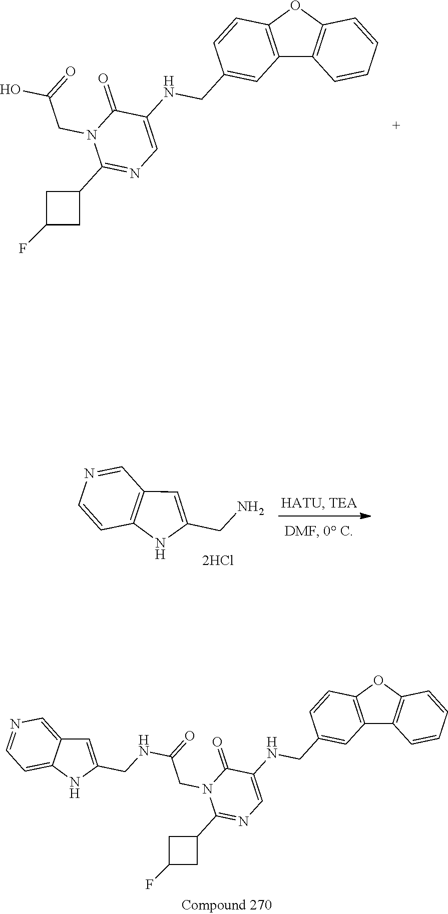 [SMILES: O=C(O)Cn1c(C2CC(F)C2)ncc(NCc2ccc3oc4ccccc4c3c2)c1=O ; NCc1cc2cnccc2[nH]1 ; "2HCl" ; O=C(Cn1c(C2CC(F)C2)ncc(NCc2ccc3oc4ccccc4c3c2)c1=O)NCc1cc2cnccc2[nH]1]

### Step 1: tert-butyl 2-(5-bromo-2-methylsulfinyl-6-oxo-pyrimidin-1-yl)acetate

A 150 mL round bottom flask was charged with tert-butyl 2-(5-bromo-2-methylsulfanyl-6-oxo-pyrimidin-1-yl)acetate (1000 mg, 2.983 mmol) and dichloromethane (20 mL) and cooled to 0° C. in a brine/ice bath. 3-chlorobenzenecarboperoxoic acid (77 wt %) (1500 mg, 6.6929 mmol) was added portion-wise, and the resulting solution was stirred at 0° C. for 45 minutes. Afterwards, the mixture was quenched with 10% sodium thiosulfate solution (50 mL) and diluted in DCM (50 mL). The organic layer was then washed with saturated bicarbonate solution (100 mL), brine (100 mL), dried over sodium sulfate, filtered, and concentrated in vacuo. Flash chromatography (RediSep Rf Gold silica, 24 g, 0-100% ethyl acetate in hexanes) afforded tert-butyl 2-(5-bromo-2-methylsulfinyl-6-oxo-pyrimidin-1-yl)acetate (830 mg, 2.363 mmol, 79.22% Yield) as a white solid. LC-MS: (ESI+) m/z=353 [M+H]⁺

### Step 2: tert-butyl 2-[5-bromo-2-(3-fluoroazetidin-1-yl)-6-oxo-pyrimidin-1-yl]acetate

A 20 mL vial was charged with tert-butyl 2-(5-bromo-2-methylsulfinyl-6-oxo-pyrimidin-1-yl)acetate (0.5M in DMF) (2.5 mL), sodium carbonate (300 mg, 2.8305 mmol), and 3-fluoroazetidine hydrochloride (200 mg, 1.7929 mmol) and heated to 60° C. for 15 minutes. Afterwards, the mixture was allowed to cool to rt, diluted in ethyl acetate (50 mL) followed by washing with water (50 mL) and brine (50 mL). The organic layer was then dried over sodium sulfate, filtered, and concentrated in vacuo to afford the crude oil. Flash chromatography (RediSep Rf Gold silica, 24 g, 0-50% ethyl acetate in hexanes) afforded tert-butyl 2-[5-bromo-2-(3-fluoroazetidin-1-yl)-6-oxo-pyrimidin-1-yl]acetate (430 mg, 1.187 mmol, 95% Yield) as a white solid. LC-MS: (ESI+) m/z=362 [M+H]⁺.

### Step 3: tert-butyl 2-[5-(dibenzofuran-2-ylmethylamino)-2-(3-fluoroazetidin-1-yl)-6-oxo-pyrimidin-1-yl]acetate

A 10 mL microwave vial was charged with dibenzofuran-2-ylmethylammonium;chloride (90 mg, 0.3851 mmol), tert-butyl 2-[5-bromo-2-(3-fluoroazetidin-1-yl)-6-oxo-pyrimidin-1-yl]acetate (75 mg, 0.2071 mmol), cesium carbonate (200 mg, 0.61384 mmol), palladium (II) acetate (5 mg, 0.02227 mmol), [1-(2-diphenylphosphanyl-1-naphthyl)-2-naphthyl]-diphenyl-phosphane (25 mg, 0.04015 mmol) and was subsequently crimp sealed and placed under nitrogen. Dioxane (2.5 mL) was added, and the resulting solution was sparged with nitrogen for 10 minutes. The vessel was then heated to 110° C. for 18 hours. Afterwards, the mixture was diluted in saturated ammonium chloride solution (10 mL) and ethyl acetate (25 mL). The organic layer was then washed with brine, dried over sodium sulfate, filtered, and concentrated in vacuo. Flash chromatography (RediSep Rf Gold silica, 24 g, 10-100% ethyl acetate in hexanes) afforded tert-butyl 2-[5-(dibenzofuran-2-ylmethylamino)-2-(3-fluoroazetidin-1-yl)-6-oxo-pyrimidin-1-yl]acetate (55 mg, 0.1149 mmol, 55.51% Yield) as a light brown solid. ¹H NMR (400 MHz, CDCl₃) δ 7.99-7.92 (m, 2H), 7.54 (dd, J=15.3, 8.3 Hz, 2H), 7.50-7.38 (m, 2H), 7.33 (t, J=7.5 Hz, 1H), 6.94 (s, 1H), 5.39-5.15 (m, 1H), 4.71 (s, 1H), 4.57 (s, 1H), 4.52 (s, 1H), 4.47-4.35 (m, 2H), 4.31-4.00 (m, 4H), 1.50 (s, 9H). LC/MS: (ESI+) m/z=479 [M+H]⁺.

### Step 4: 2-[5-(dibenzofuran-2-ylmethylamino)-2-(3-fluoroazetidin-1-yl)-6-oxo-pyrimidin-1-yl]acetic acid

tert-butyl 2-[5-(dibenzofuran-2-ylmethylamino)-2-(3-fluoroazetidin-1-yl)-6-oxo-pyrimidin-1-yl]acetate (99 mg, 0.2069 mmol) was dissolved in dichloromethane (2 mL, 31.20 mmol) at rt under ambient air. 2,2,2-trifluoroacetic acid (2 mL, 26.1 mmol) was added dropwise, and the resulting solution was stirred at rt for 4 hours. Afterwards, the mixture was concentrated in vacuo, taken up in dichloromethane (1 mL), and triturated with heptane. Removal of volatiles in vacuo afforded the crude 2-[5-(dibenzofuran-2-ylmethylamino)-2-(3-fluoroazetidin-1-yl)-6-oxo-pyrimidin-1-yl]acetic acid (100 mg, 0.2367 mmol, 114.3% Yield) as a yellow solid which was carried forward without further purification. LC/MS: (ESI+) m/z=423 [M+H]⁺.

### Step 5: 2-[5-(dibenzofuran-2-ylmethylamino)-2-(3-fluoroazetidin-1-yl)-6-oxo-pyrimidin-1-yl]-N-(1H-pyrrolo[3,2-c]pyridine-2-ylmethyl)acetamide (Compound 270)

A 20 mL vial was charged with 1H-pyrrolo[3,2-c]pyridine-2-ylmethanamine;dihydrochloride (75 mg, 0.34075 mmol), 2-[5-(dibenzofuran-2-ylmethylamino)-2-(3-fluoroazetidin-1-yl)-6-oxo-pyrimidin-1-yl]acetic acid (100 mg, 0.2367 mmol), N,N-dimethylformamide (3 mL), and triethylamine (0.200 mL, 1.51 mmol) and cooled to 0° C. in a brine/ice bath. [dimethylamino(triazolo[4,5-b]pyridine-3-yloxy)methylene]-dimethyl-ammonium;hexafluorophosphate (150 mg, 0.39450 mmol) was then added, and the resulting mixture was stirred at 0° C. for 30 minutes. Afterwards, the mixture was diluted in ethyl acetate (25 mL), washed with brine (100 mL), dried over sodium sulfate, filtered, and concentrated in vacuo. Flash chromatography (RediSep Rf Gold silica, 12 g, 0-50% MeOH in DCM) followed by prep HPLC (C18, 150×21 mm, 5 μM, 5-50% MeCN [0.1% FA] in water [0.1% FA]) and lyophilization afforded Compound 270 (19 mg, 0.03445 mmol, 14.55% Yield) as a white solid. ¹H NMR (400 MHz, DMSO) δ 11.58 (s, 1H), 8.85-8.75 (m, 2H), 8.19-8.08 (m, 4H), 7.67 (dd, J=15.5, 8.3 Hz, 2H), 7.57-7.48 (m, 2H), 7.46-7.34 (m, 2H), 6.88 (s, 1H), 6.54 (s, 1H), 5.58-5.18 (m, 1H), 4.59-4.45 (m, 4H), 4.39 (d, J=5.5 Hz, 2H), 4.26-4.12 (m, 2H), 4.02-3.85 (m, 2H). LC-MS: (ESI+) m/z=552 [M+H]⁺. RT (Method A: 1.60 min.

The following compounds were prepared based on Scheme 81:

| # | Reactant A | Reactant B | Reactant C |
|---|---|---|---|
| 260^{a,b} | 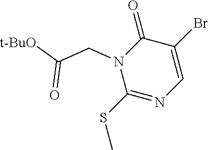 [SMILES: CSc1ncc(Br)c(=O)n1CC(=O)OC(C)(C)C] | 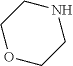 | 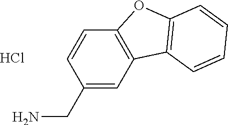 [SMILES: Cl ; NCc1ccc2oc3ccccc3c2c1] |
| 261^{a,b,c} | 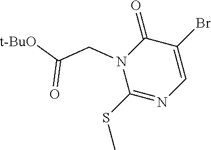 [SMILES: CSc1ncc(Br)c(=O)n1CC(=O)OC(C)(C)C] | 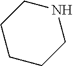 | 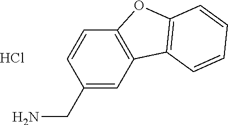 [SMILES: Cl ; NCc1ccc2oc3ccccc3c2c1] |
| 271^{c} | 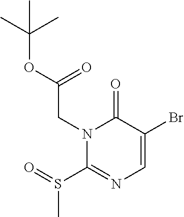 [SMILES: CS(=O)c1ncc(Br)c(=O)n1CC(=O)OC(C)(C)C] | 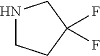 | 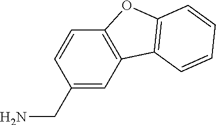 [SMILES: NCc1ccc2oc3ccccc3c2c1] |
| 281^{c} | 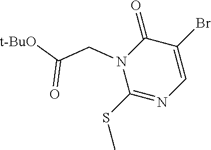 [SMILES: CSc1ncc(Br)c(=O)n1CC(=O)OC(C)(C)C] | 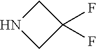 | 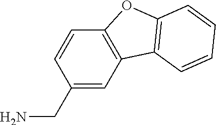 [SMILES: NCc1ccc2oc3ccccc3c2c1] |
| 304^{d} | 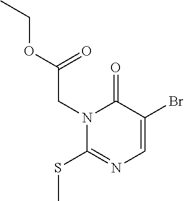 [SMILES: CCOC(=O)Cn1c(SC)ncc(Br)c1=O] | 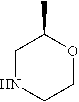 | 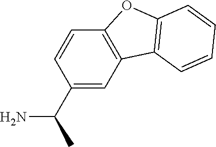 [SMILES: C[C@@H](N)c1ccc2oc3ccccc3c2c1] |
| 305^{c,d} | 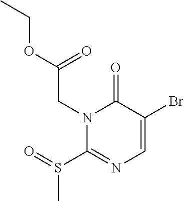 [SMILES: CCOC(=O)Cn1c(S(C)=O)ncc(Br)c1=O] | 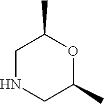 | 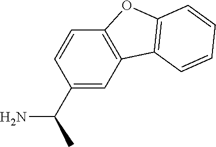 [SMILES: C[C@@H](N)c1ccc2oc3ccccc3c2c1] |
| 306^{c,d} | 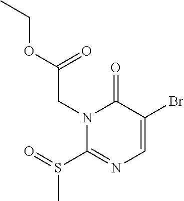 [SMILES: CCOC(=O)Cn1c(S(C)=O)ncc(Br)c1=O] | 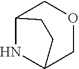 | 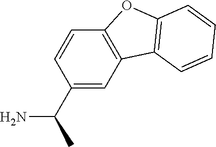 [SMILES: C[C@@H](N)c1ccc2oc3ccccc3c2c1] |
| 355^{c,d} | 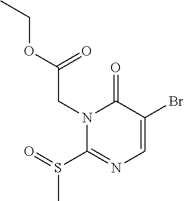 [SMILES: CCOC(=O)Cn1c(S(C)=O)ncc(Br)c1=O] | 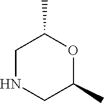 | 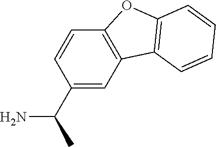 [SMILES: C[C@@H](N)c1ccc2oc3ccccc3c2c1] |
| 466^{c,d,e} | 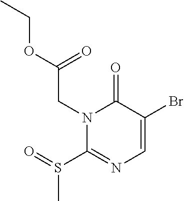 [SMILES: CCOC(=O)Cn1c(S(C)=O)ncc(Br)c1=O] | 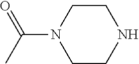 | 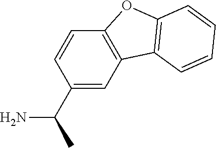 [SMILES: C[C@@H](N)c1ccc2oc3ccccc3c2c1] |
| 467^{c,d,e} | 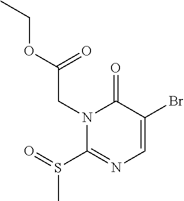 [SMILES: CCOC(=O)Cn1c(S(C)=O)ncc(Br)c1=O] | 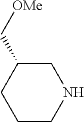 | 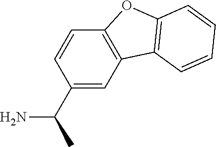 [SMILES: C[C@@H](N)c1ccc2oc3ccccc3c2c1] |
| 468^{c,d,e,f} | 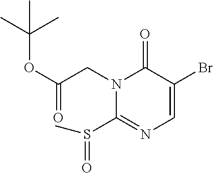 [SMILES: CS(=O)c1ncc(Br)c(=O)n1CC(=O)OC(C)(C)C] | 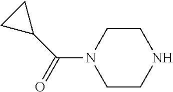 [SMILES: O=C(C1CC1)N1CCNCC1] | 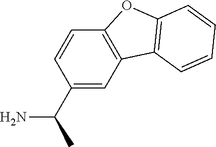 [SMILES: C[C@@H](N)c1ccc2oc3ccccc3c2c1] |
| 469^{c,d,e,f} | 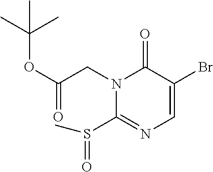 [SMILES: CS(=O)c1ncc(Br)c(=O)n1CC(=O)OC(C)(C)C] | 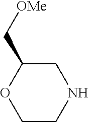 | 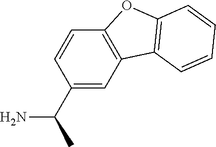 [SMILES: C[C@@H](N)c1ccc2oc3ccccc3c2c1] |
| 482^{c,d,e,f} | 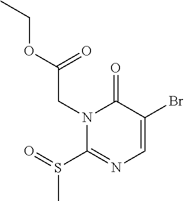 [SMILES: CCOC(=O)Cn1c(S(C)=O)ncc(Br)c1=O] | 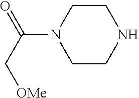 [SMILES: COCC(=O)N1CCNCC1] | 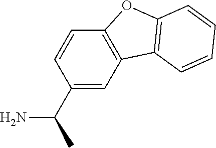 [SMILES: C[C@@H](N)c1ccc2oc3ccccc3c2c1] |

| # | Reactant D | Characterization Data |
|---|---|---|
| 260^{a,b} | 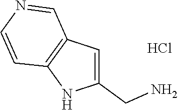 [SMILES: NCc1cc2cnccc2[nH]1 ; Cl] | ¹H NMR (400 MHz, DMSO-d₆) δ 11.29 (s, 1H), 8.82 (t, J = 5.6 Hz, 1H), 8.74 (s, 1H), 8.14 (s, 1H), 8.12 (s, 1H), 8.10 (s, 1H), 7.69 (d, J = 8.1 Hz, 1H), 7.66 (d, J = 8.6 Hz, 1H), 7.56-7.49 (m, 2H), 7.39 (t, J = 7.4 Hz, 1H), 7.33 (d, J = 5.6 Hz, 1H), |
| | | 6.89 (s, 1H), 6.45 (s, 1H), 5.95 (t, J = |
| | | 6.3 Hz, 1H), 4.72 (s, 2H), 4.47 (d, J = |
| | | 5.5 Hz, 2H), 4.42 (d, J = 6.2 Hz, |
| | | 2H), 3.54-3.50 (m, 4H), 2.80-2.77 |
| | | (m, 4H). LC/MS (ESI) m/z: 564 |
| | | (M + H)⁺. RT (Method A): 1.48 min. |
| 261^{a,b,c} | 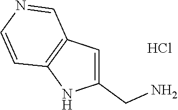 [SMILES: NCc1cc2cnccc2[nH]1 ; Cl] | ¹H NMR (400 MHz, CD₃OD) δ 8.70 (s, 1H), 8.09 (d, J = 5.9 Hz, 1H), 8.05 (s, 1H), 7.99 (d, J = 7.8 Hz, 1H), 7.57- 7.51 (m, 3H), 7.48-7.45 (m, 1H), 7.39 (d, J = 5.9 Hz, 1H), 7.34 (t, J = 7.5 Hz, 1H), 6.92 (s, 1H), 6.58 (s, 1H), 4.84 (s, 2H), 4.62 (s, 2H), 4.50 |
| | | (s, 2H), 2.88-2.84 (m, 4H), 1.54 |
| | | (dd, J = 7.2, 2.8 Hz, 4H), 1.51-1.47 |
| | | (m, 2H). LC/MS (ESI) m/z: 562 |
| | | (M + H)⁺. RT (Method A): 1.76 min. |
| 271^{c} | 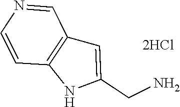 [SMILES: NCc1cc2cnccc2[nH]1 ; "2HCl"] | ¹H NMR (400 MHz, DMSO) δ 11.85 (s, 1H), 8.91 (s, 1H), 8.86 (t, J = 5.7 Hz, 1H), 8.22 (d, J = 6.1 Hz, 1H), 8.13 (s, 1H), 8.10 (d, J = 7.7 Hz, 1H), 7.66 (dd, J = 14.1, 8.3 Hz, 2H), 7.60- 7.48 (m, 3H), 7.39 (t, J = 7.5 Hz, 1H), 6.87 (s, 1H), 6.63 (s, 1H), 5.82 |
| | | (t, J = 6.3 Hz, 1H), 4.68 (s, 2H), 4.52 |
| | | (d, J = 5.6 Hz, 2H), 4.42 (d, J = 5.8 |
| | | Hz, 2H), 3.51 (t, J = 13.5 Hz, 2H), |
| | | 2.29 (tt, J = 14.4, 7.3 Hz, 2H). |
| | | LC/MS: (ESI+) m/z = 584 [M + H]⁺. |
| | | RT (Method A): 1.77 min. |
| 281^{c} | 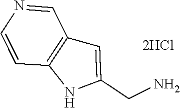 [SMILES: NCc1cc2cnccc2[nH]1 ; "2HCl"] | ¹H NMR (400 MHz, DMSO) δ 11.34 (s, 1H), 8.84-8.80 (m, 1H), 8.73 (s, 1H), 8.31 (s, 1H), 8.18-8.08 (m, 3H), 7.66 (dd, J = 15.1, 8.4 Hz, 2H), 7.58-7.46 (m, 2H), 7.39 (t, J = 7.5 Hz, 1H), 7.32 (d, J = 5.6 Hz, 1H), 6.87 (s, 1H), 6.45 (s, 1H), 5.70 (t, J = |
| | | 6.2 Hz, 1H), 4.58 (s, 2H), 4.48 (d, |
| | | J = 5.5 Hz, 2H), 4.40 (d, J = 6.2 Hz, |
| | | 2H), 4.32 (t, J = 12.9 Hz, 4H). |
| | | LC/MS: (ESI+) m/z = 570 [M + H]⁺. |
| | | RT (Method A): 1.80 min. |
| 304^{d} | 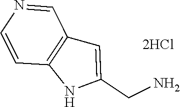 [SMILES: NCc1cc2cnccc2[nH]1 ; "2HCl"] | ¹H NMR (400 MHz, MeOD) δ 8.88 (s, 1H), 8.48 (s, 1H), 8.20 (d, J = 6.2 Hz, 1H), 8.03 (d, J = 1.7 Hz, 1H), 7.97 (d, J = 7.6 Hz, 1H), 7.64 (d, J = 6.3 Hz, 1H), 7.62-7.42 (m, 4H), 7.42-7.27 (m, 1H), 6.77 (d, J = 13.9 Hz, 2H), 4.86 (d, J = 3.2 Hz, 2H), 4.65 (d, J = |
| | | 3.9 Hz, 2H), 4.59 (q, J = 6.6 Hz, 1H), |
| | | 3.76-3.69 (m, 1H), 3.61-3.45 (m, |
| | | 2H), 2.98-2.87 (m, 2H), 2.76 (td, |
| | | J = 11.9, 3.2 Hz, 1H), 2.40 (dd, J = |
| | | 12.3, 10.1 Hz, 1H), 1.63 (d, J = 6.7 |
| | | Hz, 3H), 0.94 (d, J = 6.3 Hz, 3H). |
| | | LC/MS: (ESI+) m/z = 592 [M + H]⁺. |
| | | RT (Method A): 1.82 min. |
| 305^{c,d} | 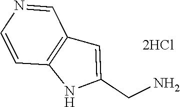 [SMILES: NCc1cc2cnccc2[nH]1 ; "2HCl"] | ¹H NMR (400 MHz, MeOD) δ 8.79 (s, 1H), 8.54 (brs, 1H), 8.15 (d, J = 6.0 Hz, 1H), 8.03 (s, 1H), 7.97 (d, J = 7.7 Hz, 1H), 7.62-7.43 (m, 5H), 7.43- 7.28 (m, 1H), 6.70 (d, J = 21.2 Hz, 2H), 4.84 (s, 2H), 4.69-4.53 (m, 3H), 3.62-3.46 (m, 2H), 2.91 (dd, |
| | | J = 25.1, 12.1 Hz, 2H), 2.31 (dt, J = |
| | | 17.0, 11.3 Hz, 2H), 1.63 (d, J = 6.6 |
| | | Hz, 3H), 0.97 (d, J = 6.2 Hz, 3H), |
| | | 0.93 (d, J = 6.2 Hz, 3H). LC/MS: |
| | | (ESI+) m/z = 606 [M + H]⁺. RT |
| | | (Method A): 1.67 min. |
| 306^{c,d} | 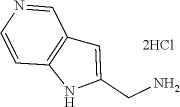 [SMILES: NCc1cc2cnccc2[nH]1 ; "2HCl"] | ¹H NMR (400 MHz, MeOD) δ 8.84 (s, 1H), 8.50 (s, 1H), 8.18 (d, J = 6.2 Hz, 1H), 8.03 (d, J = 1.7 Hz, 1H), 7.98 (d, J = 7.6 Hz, 1H), 7.61-7.42 (m, 6H), 7.33 (t, J = 7.5 Hz, 1H), 6.73 (s, 1H), 4.91 (s, 2H), 4.66 (s, 2H), 4.56 (q, J = 6.6 Hz, 1H), 3.69 (dd, J = 15.6, |
| | | 10.1 Hz, 4H), 3.51-3.38 (m, 2H), |
| | | 1.99-1.91 (m, 2H), 1.79 (d, J = 10.4 |
| | | Hz, 2H), 1.62 (d, J = 6.7 Hz, 3H). |
| | | LC/MS: (ESI+) m/z = 604.22 [M + H]⁺. |
| | | RT (Method A): 1.78 min. |
| 355^{c,d} | 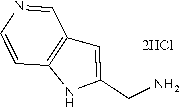 [SMILES: NCc1cc2cnccc2[nH]1 ; "2HCl"] | ¹H NMR (400 MHz, MeOD) δ 8.86 (s, 1H), 8.51 (s, 1H), 8.19 (d, J = 6.2 Hz, 1H), 8.03 (s, 1H), 7.98 (dd, J = 7.9, 3.2 Hz, 1H), 7.63-7.42 (m, 5H), 7.39-7.26 (m, 1H), 6.75 (d, J = 2.2 Hz, 2H), 4.97-4.81 (m, 2H), 4.72- 4.55 (m, 3H), 3.98-3.84 (m, 2H), |
| | | 2.93-2.83 (m, 2H), 2.68-2.49 (m, |
| | | 2H), 1.63 (d, J = 6.7 Hz, 3H), 1.11 |
| | | (dd, J = 6.5, 4.1 Hz, 6H). LC/MS: |
| | | (ESI+) m/z = 606 [M + H]⁺. RT |
| | | (Method A): 1.60 min. |
| 466^{c,d,e} | 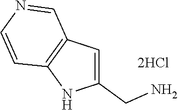 [SMILES: NCc1cc2cnccc2[nH]1 ; "2HCl"] | ¹H NMR (400 MHz, DMSO-d₆) δ 11.69 (1H, s). 8.88 (1H, t, J = 5.8 Hz), 8.84 (1H, s), 8.17 (2H, m), 8.09 (1H, d, J = 7.7 Hz), 7.68 (1H, d, J = 8.2 Hz), 7.65 (1H, d, J = 8.5 Hz), 7.58 (1H, d, J = 8.1 Hz), 7.52 (1H d, J = 7.6 Hz), 7.48 (1H, m), 7.39 (1H, t, J = |
| | | 7.5 Hz), 6.75 (1H, s), 6.56 (1H, s), |
| | | 5.56 (1H, d, J = 6.9 Hz), 4.72 (2H, |
| | | m), 4.55 (1H, m), 4.50 (2H, d, J = 5.8 |
| | | Hz), 3.27 (4H, m), 2.72 (4H, m), 1.88 |
| | | (3H, s) and 1.56 (3H, d, J = 6.7 Hz). |
| | | LC/MS (ESI) m/z: 619 (M + H)⁺. RT |
| | | (Method A): 1.48 min. |
| 467^{c,d,e} | 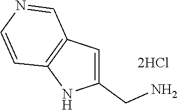 [SMILES: NCc1cc2cnccc2[nH]1 ; "2HCl"] | ¹H NMR (400 MHz, DMSO-d₆) δ 11.64 (1H, s), 8.83 (2H, m), 8.17 (2H, m), 8.09 (1H, d, J = 7.7 Hz), 7.68 (1H, d, J = 8.2 Hz), 7.64 (1H, d, J = 8.5 Hz), 7.58 (1H, m), 7.51 (1H t, J = 7.8 Hz), 7.46 (1H, d, J = 6.0 Hz), 7.39 (1H, t, J = 7.5 Hz), 6.73 (1H, s), |
| | | 6.53 (1H, s), 5.45 (1H, d, J = 6.9 Hz), |
| | | 4.65 (2H, s), 4.54 (1H, m), 4.48 (2H, |
| | | m), 3.09 (3H, s), 3.04 (2H, m), 2.95 |
| | | (2H, m), 2.46 (1H, m), 2.30 (1H, m), |
| | | 1.74 (1H, m), 1.59 (1H, m), 1.59 (3H, |
| | | d, J = 6.5 Hz), 1.41 (1H, m), 1.24 |
| | | (1H, m) and 0.99 (1H, m). LC/MS |
| | | (ESI) m/z: 620 (M + H)⁺. RT (Method |
| | | A): 1.85 min. |
| 468^{c,d,e,f} | 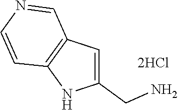 [SMILES: NCc1cc2cnccc2[nH]1 ; "2HCl"] | ¹H NMR (400 MHz, DMSO-d₆) δ11.65 (1H, s), 8.89 (1H, t, J = 5.8 Hz), 8.18 (2H, m), 8.09 (1H, d, J = 7.7 Hz), 7.68 (1H, d, J = 7.8 Hz), 7.68 (1H, d, J = 7.8 Hz), 7.64 (1H, d, J = 8.5 Hz), 7.59 (1H m), 7.51 (1H, t, J = 7.9 Hz), 7.46 (1H, d, J = 5.9 Hz), 7.39 |
| | | (1H, t, J = 7.5 Hz), 6.75 (1H, s), 6.55 |
| | | (1H, s), 5.57 (1H, d, J = 6.9 Hz), 4.73 |
| | | (2H, m), 4.55 (1H, m), 4.50 (2H, d, |
| | | J = 5.7 Hz), 3.52 (2H, m), 3.39 (2H, m), |
| | | 2.74 (4H, m), 1.80 (1H, m), 1.57 (3H, |
| | | d, J = 6.7 Hz) and 0.65 (4H, m). |
| | | LC/MS (ESI) m/z: 645 (M + H)⁺. RT |
| | | (Method A): 1.56 min. |
| 469^{c,d,e,f} | 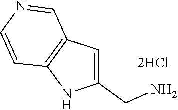 [SMILES: NCc1cc2cnccc2[nH]1 ; "2HCl"] | ¹H NMR (400 MHz, DMSO-d₆) δ 11.34 (1H, s), 8.82 (1H, t, J = 5.7 Hz)), 8.74 (1H, s), 8.18 (1H, m), 8.10 (2H, m), 7.68 (1H, d, J = 8.3 Hz), 7.64 (1H, d, J = 8.5 Hz), 7.58 (1H, m), 7.51 (1H t, J = 7.9 Hz), 7.39 (1H, t, J = 7.5 Hz), 7.33 (1H, d, J = 5.7 |
| | | Hz), 6.75 (1H, s), 6.45 (1H, s), 5.51 |
| | | (1H, d, J = 6.9 Hz), 4.73 (1H, d, J = |
| | | 5.8 Hz), 4.66 (1H, d, J = 5.8 Hz), 4.54 |
| | | (1H, m), 4.45 (2H, m), 3.63 (1H, d, |
| | | J = 11.1 Hz), 3.55 (1H, m), 3.42 (1H, t, |
| | | J = 9.8 Hz), 3.23 (1H, m), 3.17 (1H, |
| | | m), 3.15 (3H, s), 2.88 (2H, m), 2.57 |
| | | (1H, m), 2.43 (1H, m) and 1.59 (3H, |
| | | d, J = 6.5 Hz). LC/MS (ESI) m/z: 622 |
| | | (M + H)⁺. RT (Method A): 1.55 min. |
| 482^{c,d,e,f} | 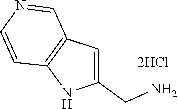 [SMILES: NCc1cc2cnccc2[nH]1 ; "2HCl"] | ¹H NMR (400 MHz, DMSO-d₆) δ11.35 (1H, s), 8.84 (1H, t, J = 5.7 Hz), 8.73 (1H, s), 8.17 (1H, s), 8.09 (2H, m), 7.68 (1H, d, J = 8.2 Hz), 7.64 (1H, d, J = 8.4 Hz), 7.58 (1H, m), 7.51 (1H, t, J = 8.2 Hz), 7.39 (1H, d, J = 7.5 Hz), 7.33 (1H, d, J = 5.7 |
| | | Hz), 6.75 (1H, s), 6.45 (1H, s), 5.57 |
| | | (1H, d, J = 6.9 Hz), 4.71 (2H, s), 4.55 |
| | | (1H, m), 4.47 (2H, m), 3.95 (2H, s), |
| | | 3.39 (2H, m), 3.22 (2H, m), 3.20 (3H, |
| | | s), 2.74 (4H, m) and 1.59 (3H, d, J = |
| | | 6.5 Hz). LC/MS (ESI) m/z: 649 |
| | | (M + H)⁺. RT (Method A): 1.64 min. |

^{a}Step 2 was performed in 1,4-dioxane.
^{b}Step 3 was performed with Pd₂(dba)₃ and BrettPhos in toluene in the presence of CS₂CO₃.
^{c}Steps 2-5 only.
^{d}Step 4 was performed with NaOH in THE/MeOH.
^{e}Step 3 was performed in toluene.
^{f}Step 2 was performed with DIPEA in DMF.

### Scheme 82. Synthesis of 6-(2-(((1H-pyrrolo[3,2-c]pyridine-2-yl)methyl)amino)-2-oxoethoxy)-N-(dibenzo[b,d]furan-2ylmethyl)-2-phenylpyrimidine-4-carboxamide (Compound 268)

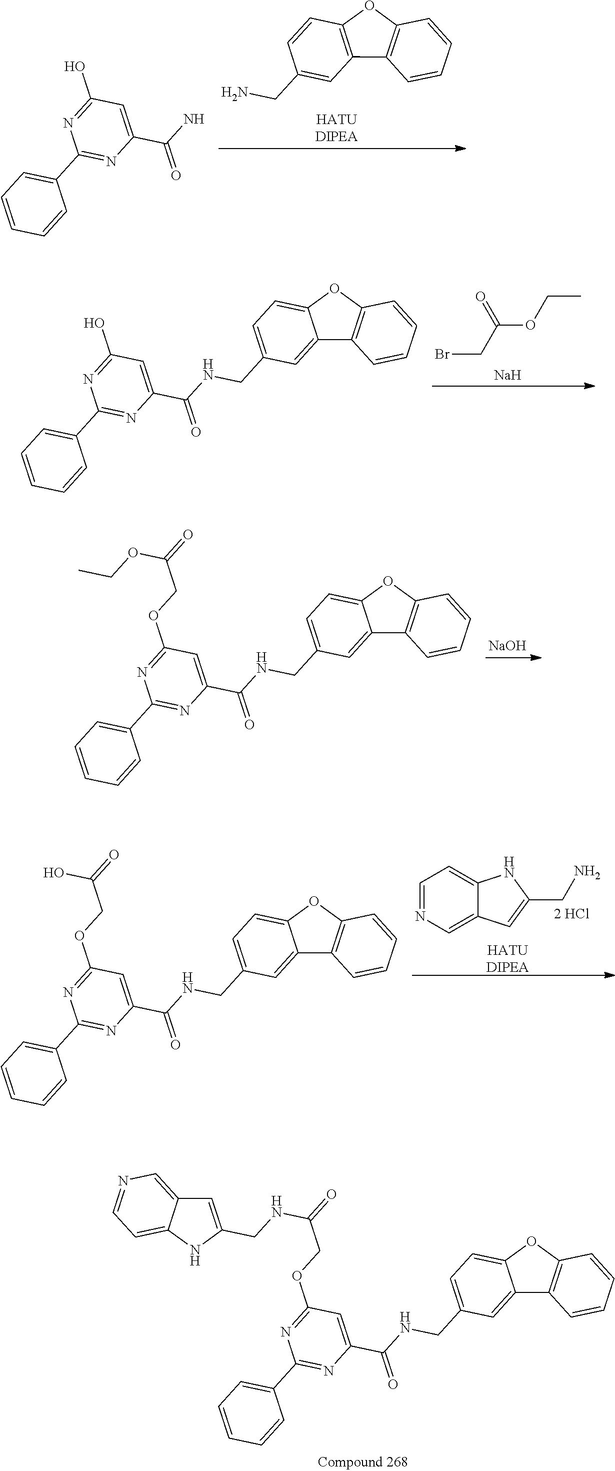

### Step 1: N-(dibenzo[b,d]furan-2-ylmethyl)-6-hydroxy-2-phenylpyrimidine-4-carboxamide

To a suspension of 6-hydroxy-2-phenyl-pyrimidine-4-carboxylic acid (100 mg, 0.46 mmol), dibenzofuran-2-ylmethanamine (91 mg, 0.46 mmol), and HATU (264 mg, 0.69 mmol) in DMF (2 mL) was added DIPEA (0.24 mL, 1.4 mmol) at room temperature. The reaction was stirred for 1 hour and diluted with water. The solid was collected by filtration to give N-(dibenzo[b,d]furan-2-ylmethyl)-6-hydroxy-2-phenylpyrimidine-4-carboxamide (57 mg, 31% yield) as a white solid. LC/MS (ESI) m/z: 396 (M+H)⁺.

### Step 2: Ethyl 2-((6-((dibenzo[b,d]furan-2-ylmethyl)carbamoyl)-2-phenylpyrimidin-4-yl)oxy)acetate

To a solution of N-(dibenzo[b,d]furan-2-ylmethyl)-6-hydroxy-2-phenylpyrimidine-4-carboxamide (57 mg, 0.14 mmol) in DMF (2 mL) at 0° C. was added NaH (60% in mineral oil, 37 mg, 0.93 mmol). The reaction was stirred for 30 minutes, then treated with ethyl 2-bromoacetate (0.1 mL, 0.9 mmol). The reaction was stirred at 50° C. for 3 hours. The mixture was quenched with water and extracted with EtOAc (3×10 mL), the combined organic layers were washed with brine, dried over Na₂SO₄, filtered and concentrated. The residue was purified by silica gel column chromatography (0 to 50% EtOAc in hexanes) to give ethyl 2-((6-((dibenzo[b,d]furan-2-ylmethyl)carbamoyl)-2-phenylpyrimidin-4-yl)oxy)acetate (50 mg, 22% yield) as a white solid. LC/MS (ESI) m/z: 482 (M+H)⁺.

### Step 3: 2-((6-((Dibenzo[b,d]furan-2-ylmethyl)carbamoyl)-2-phenylpyrimidin-4-yl)oxy)acetic acid

To a solution of ethyl 2-((6-((dibenzo[b,d]furan-2-ylmethyl)carbamoyl)-2-phenylpyrimidin-4-yl)oxy)acetate (50 mg, 0.10 mmol) in a 1:1:1 mixture of THF/MeOH/H₂O (1 mL) was added 1N NaOH aq. (0.24 mL, 0.24 mmol) at room temperature. The reaction was stirred for 1 hour and acidified with 1 N HCl aq. To pH 4-5. The mixture was extracted with EtOAc (3×10 mL). The combined organic layers were dried and concentrated to give 2-[6-(dibenzofuran-2-ylmethylcarbamoyl)-2-phenyl-pyrimidin-4-yl]oxyacetic acid (50 mg, >99% yield) as a clear oil. LC/MS (ESI) m/z: 454 (M+H)⁺.

### Step 4: 6-(2-(((1H-pyrrolo[3,2-c]pyridine-2-yl)methyl)amino)-2-oxoethoxy)-N-(dibenzo[b,d]furan-2-ylmethyl)-2-phenylpyrimidine-4-carboxamide (Compound 268)

To an ice-bath cooled solution of 1H-pyrrolo[3,2-c]pyridine-2-yl)methanamine dihydrochloride (25 mg, 0.14 mmol), 2-[6-(dibenzofuran-2-ylmethylcarbamoyl)-2-phenyl-pyrimidin-4-yl]oxyacetic acid (50 mg, 0.11 mmol), HATU (63 mg, 0.17 mmol) in DMF (2 mL) was added DIPEA (0.1 mL, 0.6 mmol). The reaction was stirred for 10 minutes and quenched with water. The solid was collected by filtration and purified by an ACCQ Prep-HPLC (0 to 100% MeCN in H₂O (containing 0.05% HCOOH)) to give Compound 268 (35 mg, 55% yield) as an off-white solid. ¹H-NMR (400 MHz, DMSO-d₆) δ 11.30 (s, 1H), 9.78 (t, J=6.4 Hz, 1H), 8.82 (t, J=5.8 Hz, 1H), 8.70-8.53 (m, 2H), 8.46 (s, 1H), 8.22-8.01 (m, 2H), 7.67 (dd, J=8.4, 5.9 Hz, 2H), 7.61-7.33 (m, 5H), 7.25 (d, J=5.6 Hz, 1H), 6.29 (s, 1H), 5.09 (s, 2H), 4.74 (d, J=6.4 Hz, 2H), 4.48 (d, J=5.8 Hz, 2H). LC/MS (ESI) m/z: 583 (M+H)⁺. RT (Method A): 1.86 min.

### Scheme 83. Synthesis of N-((1H-pyrrolo[3,2-c]pyridine-2-yl)methyl)-2-((5-((dibenzo[b,d]furan-2-ylmethyl)amino)-6-methyl-2-phenylpyrimidin-4-yl)oxy)acetamide (Compound 269)

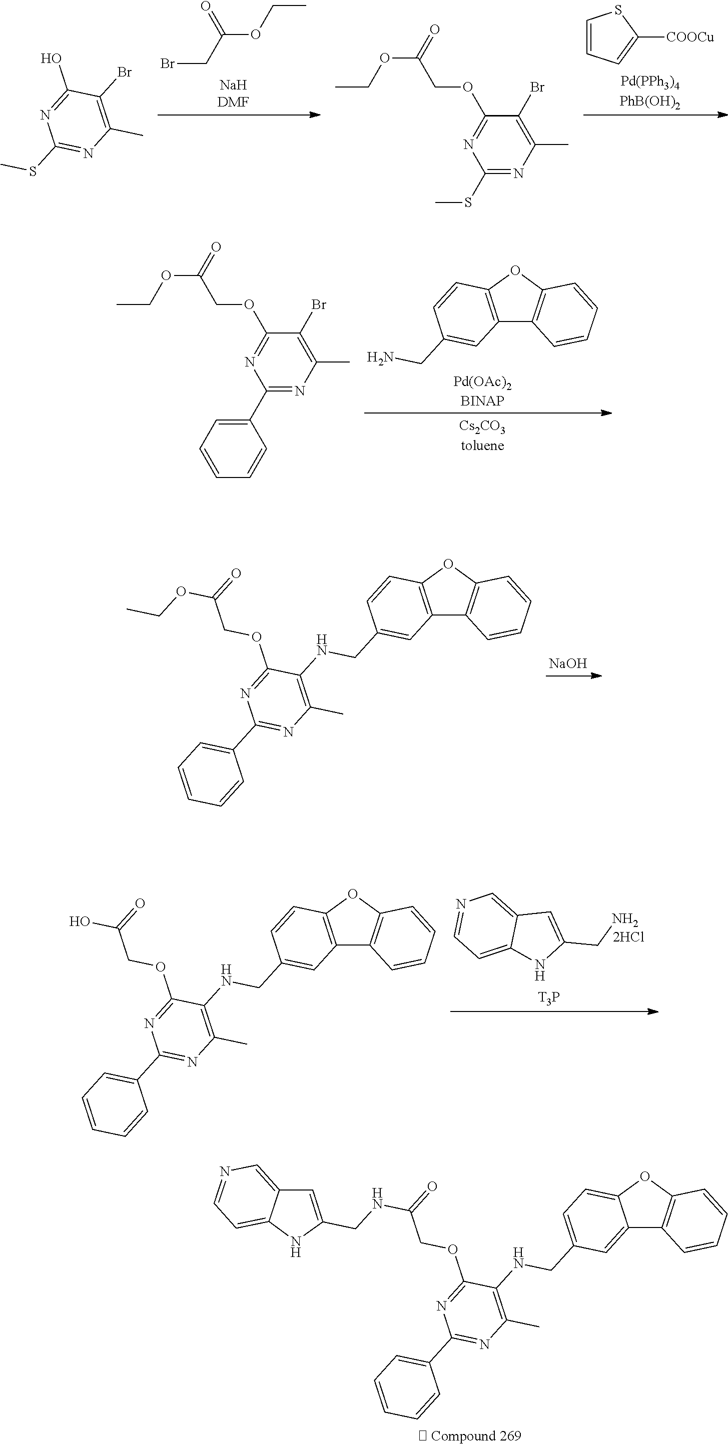

### Step 1: Ethyl 2-((5-bromo-6-methyl-2-(methylthio)pyrimidin-4-yl)oxy)acetate

To an ice-bath cooled solution of 5-bromo-6-methyl-2-methylsulfanyl-pyrimidin-4-ol (750 mg, 3.19 mmol, in DMF (2 mL) was added ethyl 2-bromoacetate (0.71 mL, 6.4 mmol) at 0° C. The reaction was then stirred at room temperature overnight. The reaction was diluted with water and extracted with EtOAc (3×20 mL). The organic layers were combined and concentrated. The residue was purified by silica gel column chromatography (0 to 50% EtOAc in hexanes) to give ethyl 2-((5-bromo-6-methyl-2-(methylthio)pyrimidin-4-yl)oxy)acetate (400 mg, 39 yield) as a colorless oil. LC/MS (ESI) m/z: 322, 324 (M+H)⁺.

### Step 2: Ethyl 2-((5-bromo-6-methyl-2-phenylpyrimidin-4-yl)oxy)acetate

A mixture of ethyl 2-((5-bromo-6-methyl-2-(methylthio)pyrimidin-4-yl)oxy)acetate (100 mg, 0.31 mmol), copper(I)-thiophene-2-carboxylate (89 mg, 0.47 mmol), Pd(PPh₃)₄ (36 mg, 0.031 mmol), and phenylboronic acid (47 mg, 0.39 mmol) in THF (2 mL) was sparged with nitrogen for 10 minutes, then heated to reflux for 18 hours. The mixture was diluted with EtOAc (5 mL), and the solid was removed by filtration. The filtrate was concentrated and purified by silica gel column chromatography (0 to 50% EtOAc in hexanes) to give ethyl 2-((5-bromo-6-methyl-2-phenylpyrimidin-4-yl)oxy)acetate (65 mg, 60% yield) as a white solid. LC/MS (ESI) m/z: 351, 353 (M+H)⁺.

### Step 3: Ethyl 2-((5-((dibenzo[b,d]furan-2-ylmethyl)amino)-6-methyl-2-phenylpyrimidin-4-yl)oxy)acetate

A mixture of ethyl 2-((5-bromo-6-methyl-2-phenylpyrimidin-4-yl)oxy)acetate (65 mg, 0.19 mmol), dibenzofuran-2-ylmethanamine (154 mg, 0.789 mmol), Pd(OAc)₂ (7 mg, 0.031 mmol), Cs₂CO₃ (152 mg, 0.47 mmol), and BINAP (39 mg, 0.063 mmol) in toluene (2 mL) was sparged with nitrogen for 10 minutes, then heated to 110° C. for 18 hours. The reaction mixture was diluted with EtOAc (10 mL), and the solid was removed by filtration through a Celite pad. The residue was purified by silica gel column chromatography (0 to 50% EtOAc in hexanes) to give ethyl 2-((5-((dibenzo[b,d]furan-2-ylmethyl)amino)-6-methyl-2-phenylpyrimidin-4-yl)oxy)acetate (60 mg, 69% yield) as a brown solid. LC/MS (ESI) m/z: 468 (M+H)⁺.

### Step 4: 2-((5-((Dibenzo[b,d]furan-2-ylmethyl)amino)-6-methyl-2-phenylpyrimidin-4-yl)oxy)acetic acid

To a solution of ethyl 2-((5-((dibenzo[b,d]furan-2-ylmethyl)amino)-6-methyl-2-phenylpyrimidin-4-yl)oxy)acetate (50 mg, 0.11 mmol) in a 1:1:1 mixture of MeOH/THF/H₂O (1 mL) was added NaOH (15 mg, 0.38 mmol) at room temperature. After stirring for 1 hour, the reaction was acidified to pH 4 and extracted with EtOAc (3×20 mL). The organic layer was washed with brine, dried over Na₂SO₄, filtered, and concentrated to give 2-((5-((Dibenzo[b,d]furan-2-ylmethyl)amino)-6-methyl-2-phenylpyrimidin-4-yl)oxy)acetic acid (50 mg, 89% yield) as a waxy solid. LC/MS (ESI) m/z: 440 (M+H)⁺.

### Step 5: N-((1H-pyrrolo[3,2-c]pyridine-2-yl)methyl)-2-((5-((dibenzo[b,d]furan-2-ylmethyl)amino)-6-methyl-2-phenylpyrimidin-4-yl)oxy)acetamide (Compound 269)

To an ice-bath cooled mixture of 2-((5-((Dibenzo[b,d]furan-2-ylmethyl)amino)-6-methyl-2-phenylpyrimidin-4-yl)oxy)acetic acid (50 mg, 0.11 mmol), 1H-pyrrolo[3,2-c]pyridine-2-yl)methanamine dihydrochloride (28 mg, 0.15 mmol), and HATU (65 mg, 0.17 mmol) in DMF (2 mL) was added DIPEA (0.1 mL, 0.6 mmol). After stirring for 20 minutes, H₂O was added. The precipitates were collected by filtration and dissolved in DMSO (3 mL), which was purified by an ACCQ Prep-HPLC (0 to 100% MeCN in H₂O (contains 0.05% HCOOH)) to give Compound 269 (5 mg, 8% yield) as an off-white solid. ¹H-NMR (400 MHz, DMSO-d₆) δ 11.37 (s, 1H), 8.80 (t, J=5.8 Hz, 1H), 8.50 (s, 1H), 8.32-8.13 (m, 2H), 8.06 (t, J=6.6 Hz, 2H), 7.64 (d, J=8.2 Hz, 1H), 7.61-7.51 (m, 3H), 7.47 (t, J=7.8 Hz, 1H), 7.41-7.21 (m, 5H), 6.36 (s, 1H), 5.44 (d, J=7.2 Hz, 2H), 5.01 (s, 2H), 4.58 (m, 2H), 2.44 (s, 3H). LC/MS (ESI) m/z: 569 (M+H)⁺. RT (Method A): 1.83 min.

### Scheme 84. Synthesis of (S)—N-((1H-pyrrolo[3,2-c]pyridine-2-yl)methyl)-2-(5-((2,3-dihydro-1H-inden-1-yl)amino)-2-(2-fluorophenyl)-6-oxopyrimidin-1(6H)-yl)acetamide (Compound 394)

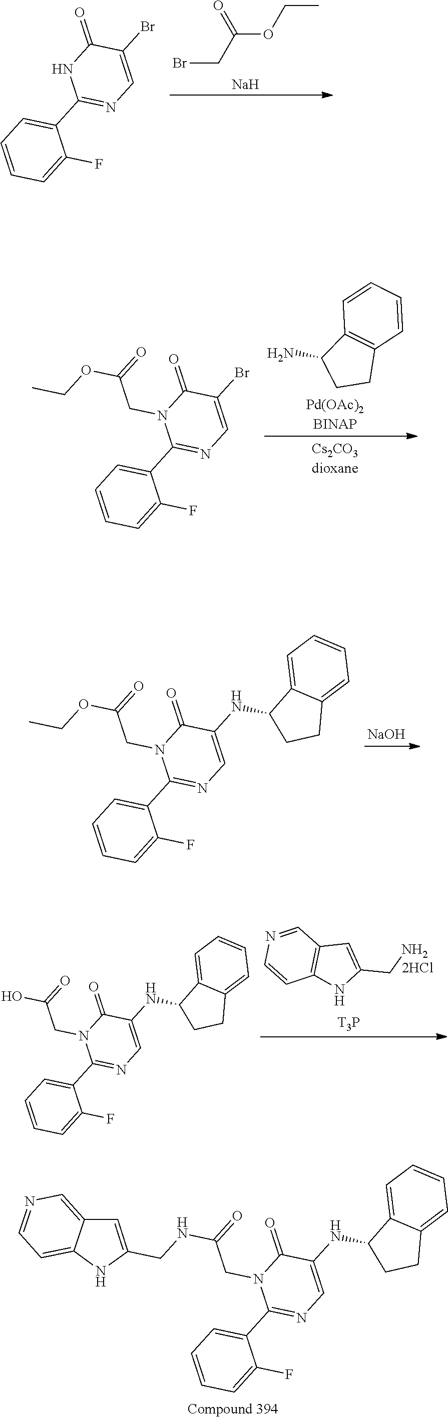

### Step 1: Ethyl 2-(5-bromo-2-(2-fluorophenyl)-6-oxopyrimidin-1(6H)-yl)acetate

To an ice-bath cooled suspension of NaH (60% suspension in mineral oil, 480 mg, 12 mmol) in THF (20 mL) was added 5-bromo-2-(2-fluorophenyl)-1H-pyrimidin-6-one (1.6 g, 5.9 mmol) in THF (20 mL). The mixture was stirred for 30 minutes, then treated with ethyl 2-bromoacetate (1.3 mL, 12 mmol). The resulting mixture was stirred at room temperature overnight. Extra NaH (60% suspension in mineral oil, 488 mg) and ethyl bromoacetate (1.3 mL) was added and the stirring continued for additional 3 days. The mixture was quenched with ice-water and extracted with EtOAc (3×50 mL). The organic layer was washed with brine, dried over Na₂SO₄, filtered, and concentrated. The residue was purified by silica gel column chromatography (0 to 100% EtOAc in hexanes) to give ethyl 2-[5-bromo-2-(2-fluorophenyl)-6-oxo-pyrimidin-1-yl]acetate (1.4 g, 66% yield) as a yellow oil. LC/MS (ESI) m/z: 355, 357 (M+H)⁺.

### Step 2: Ethyl (S)-2-(5-((2,3-dihydro-1H-inden-1-yl)amino)-2-(2-fluorophenyl)-6-oxopyrimidin-1(6H)-yl)acetate

A mixture of ethyl 2-[5-bromo-2-(2-fluorophenyl)-6-oxo-pyrimidin-1-yl]acetate (150 mg, 0.42 mmol), (1S)-indan-1-amine (113 mg, 0.85 mmol), Pd(OAc)₂ (10 mg, 0.044 mmol), BINAP (53 mg, 0.085 mmol), and Cs₂CO₃ (413 mg, 1.27 mmol) in 1,4-dioxane (4 mL, 46.85 mmol) was sparged with nitrogen for 10 minutes, then heated to 110° C. for 2 hours. The reaction mixture was diluted with EtOAc (10 mL) and filtered through a pad of Celite. The filtrate was concentrated and purified on silica gel chromatography (0 to 50% EtOAc in hexanes) to ethyl (S)-2-(5-((2,3-dihydro-1H-inden-1-yl)amino)-2-(2-fluorophenyl)-6-oxopyrimidin-1(6H)-yl)acetate (200 mg, 116% yield) as a dark oil, which was of sufficient purity to continue as is, without further purification. LC/MS (ESI) m/z: 408 (M+H)⁺.

### Step 3: (S)-2-(3-((2,3-dihydro-1H-inden-1-yl)amino)-6-(2-fluorophenyl)-2-oxopyridin-1(2H)-yl)acetic acid

To a solution of ethyl (S)-2-(5-((2,3-dihydro-1H-inden-1-yl)amino)-2-(2-fluorophenyl)-6-oxopyrimidin-1(6H)-yl)acetate (200 mg, 0.49 mmol) in MeOH (2 mL) and THF (4 mL) was added 1N NaOH aq. (1 mL) at room temperature. The reaction was stirred for overnight. Added H₂O (20 mL) and washed with EtOAc (10 mL). The organic layer was discarded. The aqueous layer was acidified to pH=3 with 1 N HCl aq and extracted with EtOAc (3×20 mL). The combined organic layers were washed with brine, dried over Na₂SO₄, filtered, and concentrated to give (S)-2-(3-((2,3-dihydro-1H-inden-1-yl)amino)-6-(2-fluorophenyl)-2-oxopyridin-1(2H)-yl)acetic acid (138 mg, 74% yield) as a yellow foamy solid. LC/MS (ESI) m/z: 380 (M+H)⁺.

### Step 4: (S)—N-((1H-pyrrolo[3,2-c]pyridine-2-yl)methyl)-2-(5-((2,3-dihydro-1H-inden-1-yl)amino)-2-(2-fluorophenyl)-6-oxopyrimidin-1(6H)-yl)acetamide (Compound 394)

To a mixture of (S)-2-(3-((2,3-dihydro-1H-inden-1-yl)amino)-6-(2-fluorophenyl)-2-oxopyridin-1(2H)-yl)acetic acid (130 mg, 0.34 mmol), 1H-pyrrolo[3,2-c]pyridine-2-ylmethanamine dihydrochloride (83 mg, 0.38 mmol), 50% T₃P in EtOAc (0.26 mL, 0.87 mmol) in DMF (1.3 mL) at 0° C. was added DIPEA (0.3 mL, 2 mmol). The reaction was kept at 0° C. for 30 minutes then quenched with saturated NaHCO₃ aq. (2 mL) and diluted with DMSO (2 mL). The solid was removed by filtration over a Celite pad. The filtrate was purified with an ACCQ prep-HPLC system (0 to 100% MeCN in H₂O (contains 0.05% HCOOH)) to give Compound 394 (50 mg, 29% yield) as an off-white solid. ¹H-NMR (400 MHz, MeOD) δ 8.86 (s, 1H), 8.53 (s, 1H), 8.31-8.05 (m, 1H), 7.65 (d, J=5.5 Hz, 1H), 7.57-7.40 (m, 4H), 7.39-7.10 (m, 9H), 6.65 (s, 1H), 5.02 (t, J=7.0 Hz, 1H), 4.60-4.35 (m, 3H), 3.10-2.83 (m, 2H), 2.58 (dtd, J=12.1, 7.6, 4.1 Hz, 1H), 1.97 (dq, J=12.7, 7.9 Hz, 1H). LC/MS (ESI) m/z: 509 (M+H)⁺. RT (Method A): 1.33 min.

### Scheme 85. Synthesis of (R)—N-((1H-pyrrolo[3,2-c]pyridine-2-yl)methyl)-2-(5-((1-(6-fluorodibenzo[b,d]furan-2-yl)ethyl)amino)-2-(2-fluorophenyl)-6-oxopyrimidin-1(6H)-yl)acetamide (Compound 318)

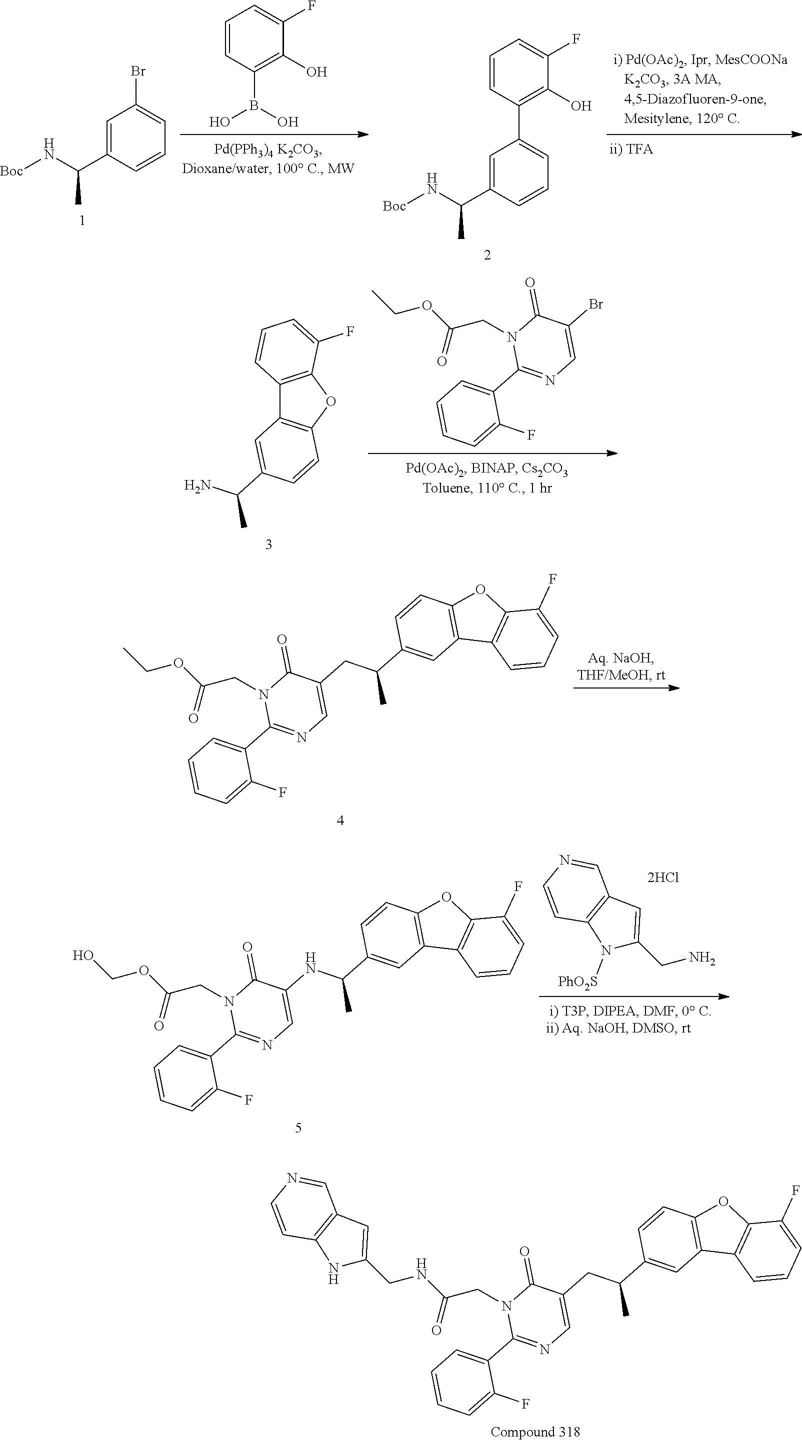

### Step 1: tert-Butyl (R)-(1-(3′-fluoro-2′-hydroxy-[1,1′-biphenyl]-3-yl)ethyl)carbamate

tert-Butyl-N-[(1R)-1-(3-bromophenyl)ethyl]carbamate (0.2 g, 0.67 mmol), (3-fluoro-2-hydroxy-phenyl)boronic acid (0.2493 g, 1.599 mmol), Tetrakis(Triphenylphosphine)Palladium(0) (0.07699 g, 0.06662 mmol), K₂CO₃ (0.18 g, 1.2 mmol) in 1,4-Dioxane (1.6 mL) and water (0.4 mL) were transferred to a microwave vial and the mixture was purged with nitrogen for 3 minutes. Reaction mixture was heated under microwave irradiation at 100° C. for 10 min. Crude mixture was purified using ISCO (20% EtOAc/Hexane) to give tert-butyl (R)-(1-(3′-fluoro-2′-hydroxy-[1,1′-biphenyl]-3-yl)ethyl)carbamate (168 mg, 80% Yield). LC/MS (ESI) m/z: 332 (M+H)⁺.

### Step 2: (R)-1-(6-Fluorodibenzo[b,d]furan-2-yl)ethan-1-amine

A vial was charged with tert-Butyl N-[(1R)-1-[3-(3-fluoro-2-hydroxy-phenyl)phenyl]ethyl]-carbamate (0.28 g, 0.8449 mmol), Palladium(II) Acetate (0.009484 g, 0.04225 mmol), 1,3-bis(2,6-Diisopropylphenyl)-1H-imidazol-3-ium-2-ide (0.03385 g, 0.08449 mmol), 4,5-Diazafluoren-9-one (0.01539 g, 0.08449 mmol), Sodium 2,4,6-trimethylbenzoate (0.07865 g, 0.4225 mmol), K₂CO₃ (0.2335 g, 1.690 mmol), 3 A MS (0.5 g) and mesitylene. The vial was stirred at room temperature for 1 minute and then stirred at the 120° C. under air for 18 hr. The mixture was diluted with EtOAc and filtered, and the combined filtrate was concentrated under vacuum. The crude material was purified by flash chromatography (15% EA/Hexane)) to give intermediate which was dissolved in DCM. TFA was added and allowed to stir for 30 minutes. Solvent and excess TFA were evaporated. Crude mass was extracted with EtOAc and saturated sodium bicarbonate solution. Organic layer was isolated, dried over sodium sulfate and evaporated to dryness to give (R)-1-(6-fluorodibenzo[b,d]furan-2-yl)ethan-1-amine (46 mg, 17% Yield). LC/MS (ESI) m/z: 230 (M+H)⁺.

### Step 3: Ethyl (R)-2-(5-((1-(6-fluorodibenzo[b,d]furan-2-yl)ethyl)amino)-2-(2-fluorophenyl)-6-oxopyrimidin-1(6H)-yl)acetate

A vial was charged with ethyl 2-[5-bromo-2-(2-fluorophenyl)-6-oxo-pyrimidin-1-yl]acetate (0.03099 g, 0.08726 mmol), (R)-1-(6-fluorodibenzo[b,d]furan-2-yl)ethan-1-amine (0.02 g, 0.08726 mmol), Cesium carbonate (0.0569 g, 0.1745 mmol), (R)-(+)-2,2′-bis-(Diphenyl-phosphino)-1,1′-Binaphthyl (0.01087 g, 0.01745 mmol) and Palladium(II) Acetate (0.001959 g, 0.008726 mmol). Toluene was added and the reaction mixture was purged with nitrogen for 5 minutes. Reaction mixture was allowed to stir at 110° C. for 1 hr. The crude mixture was purified using flash chromatography (silica gel, 20-30% EtOAc/Hexane) to give ethyl (R)-2-(5-((1-(6-fluorodibenzo[b,d]furan-2-yl)ethyl)amino)-2-(2-fluorophenyl)-6-oxopyrimidin-1(6H)-yl)acetate (23 mg, 52.4% Yield). LC/MS (ESI) m/z: 504 (M+H)⁺.

### Step 4: (R)-2-(5-((1-(6-Fluorodibenzo[b,d]furan-2-yl)ethyl)amino)-2-(2-fluorophenyl)-6-oxopyrimidin-1(6H)-yl)acetic acid

Ethyl (R)-2-(5-((1-(6-fluorodibenzo[b,d]furan-2-yl)ethyl)amino)-2-(2-fluorophenyl)-6-oxo-pyrimidin-1(6H)-yl)acetate (23 mg, 0.046 mmol) was dissolved in the (1:5) mixture of THF/MeOH. Sodium hydroxide (1 mol/L) in water (0.138 mL, 0.138 mmol) was added dropwise into the stirring reaction mixture and allowed to stir for 45 minutes. The reaction mixture was acidified with 1 N HCl. All solvents were evaporated to complete dryness to give (R)-2-(5-((1-(6-fluorodibenzo-[b,d]furan-2-yl)ethyl)amino)-2-(2-fluorophenyl)-6-oxopyrimidin-1(6H)-yl)acetic acid (21 mg, 96.70% Yield) which was used in the next step without further purification. LC/MS (ESI) m/z: 476 (M+H)⁺.

### Step 5: (R)—N-((1H-Pyrrolo[3,2-c]pyridine-2-yl)methyl)-2-(5-((1-(6-fluorodibenzo[b,d]furan-2-yl)ethyl)amino)-2-(2-fluorophenyl)-6-oxopyrimidin-1(6H)-yl)acetamide (Compound 318)

N,N-Diisopropylethylamine (0.02855 g, 0.0385 mL, 0.2209 mmol) was added to a cold (0° C.) stirred solution of the (R)-2-(5-((1-(6-fluorodibenzo[b,d]furan-2-yl)ethyl)amino)-2-(2-fluoro-phenyl)-6-oxopyrimidin-1(6H)-yl)acetic acid (0.021 g, 0.04417 mmol) and [1-(benzenesulfonyl)-pyrrolo[3,2-c]pyridine-2-yl]methanamine;dihydrochloride (0.06154 g, 0.1708 mmol) in dry DMF. T₃P (1.67 mol/L) in ethyl acetate (0.0794 mL, 0.1325 mmol) was added and the reaction was stirred at 0° C. for 40 mins. Water was added into the reaction mixture and precipitates were collected. Crude material was dissolved in DMSO (minimum quantity) and sodium hydroxide (1 mol/L) in water (0.22 mL, 0.22 mmol) was added into the stirring solution. Reaction was monitored by LC/MS until complete. Crude reaction mixture was purified using HPLC to give Compound 318 (3 mg, 8.9% Yield). ¹H NMR (400 MHz, MeOD) δ 8.05 (s, 1H), 7.78 (d, J=7.6 Hz, 1H), 7.70 (s, 3H), 7.65-7.53 (m, 2H), 7.46 (s, 1H), 7.40-7.05 (m, 6H), 6.93 (s, 1H), 6.69 (s, 1H), 4.97 (s, 1H), 4.69-4.42 (m, 4H), 1.70 (d, J=6.7 Hz, 3H). LC/MS (ESI) m/z: 605 (M+H)⁺. RT (Method A): 1.70 min.

The following compounds were prepared based on Scheme 85:

| # | Reactant A | Reactant B | Reactant C | Reactant D |
|---|---|---|---|---|
| 352 | 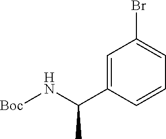 [SMILES: C[C@@H](NC(=O)OC(C)(C)C)c1cccc(Br)c1] | 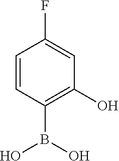 [SMILES: OB(O)c1ccc(F)cc1O] | 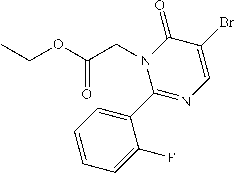 [SMILES: CCOC(=O)Cn1c(-c2ccccc2F)ncc(Br)c1=O] | 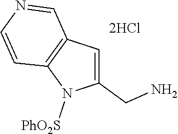 |
| 353 | 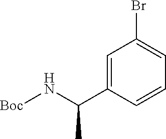 [SMILES: C[C@@H](NC(=O)OC(C)(C)C)c1cccc(Br)c1] | 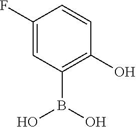 [SMILES: OB(O)c1cc(F)ccc1O] | 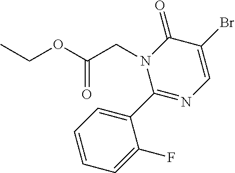 [SMILES: CCOC(=O)Cn1c(-c2ccccc2F)ncc(Br)c1=O] | 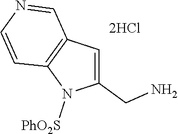 |
| 354 | 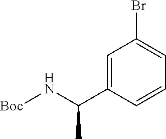 [SMILES: C[C@@H](NC(=O)OC(C)(C)C)c1cccc(Br)c1] | 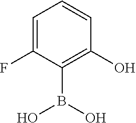 [SMILES: OB(O)c1c(O)cccc1F] | 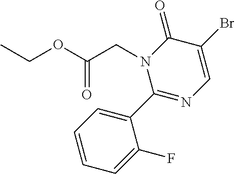 [SMILES: CCOC(=O)Cn1c(-c2ccccc2F)ncc(Br)c1=O] | 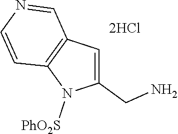 |
| 396 | 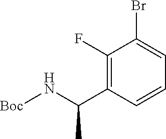 [SMILES: C[C@@H](NC(=O)OC(C)(C)C)c1cccc(Br)c1F] | 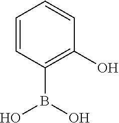 | 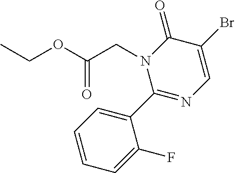 [SMILES: CCOC(=O)Cn1c(-c2ccccc2F)ncc(Br)c1=O] | 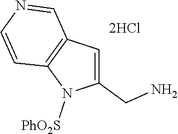 |
| | | | # | Characterization Data |
| | | | 352 | ¹H NMR (400 MHz, DMSO) δ 11.35 (s, |
| | | | | 1H), 8.87-8.51 (m, 2H), 8.33-7.94 |
| | | | | (m, 4H), 7.77-7.45 (m, 4H), 7.43- |
| | | | | 7.19 (m, 4H), 7.14 (t, J = 7.5 Hz, 1H), |
| | | | | 6.99 (s, 1H), 6.24 (s, 1H), 6.04 (d, J = |
| | | | | 7.1 Hz, 1H), 4.67 (t, J = 6.9 Hz, 1H), |
| | | | | 4.33 (d, J = 5.6 Hz, 3H), 1.61 (d, J = |
| | | | | 6.7 Hz, 3H). LC/MS (ESI) m/z: 605 |
| | | | | (M + H)⁺. RT (Method A): 1.78 min. |
| | | | 353 | ¹H NMR (400 MHz, MeOD) δ 8.91 (s, |
| | | | | 1H), 8.22 (s, 2H), 7.99 (s, 1H), 7.78 (s, |
| | | | | 1H), 7.65 (d, J = 7.8 Hz, 1H), 7.59- |
| | | | | 7.42 (m, 5H), 7.37 (s, 1H), 7.23 (t, J = |
| | | | | 7.5 Hz, 3H), 6.94 (s, 1H), 6.76 (s, 1H), |
| | | | | 4.97 (s, 1H), 4.65 (q, J = 6.8 Hz, 1H), |
| | | | | 4.55 (s, 1H), 4.36 (s, 1H), 1.70 (d, J = |
| | | | | 6.7 Hz, 3H). LC/MS (ESI) m/z: 605 |
| | | | | (M + H)⁺. RT (Method A): 1.70 min. |
| | | | 354 | ¹H NMR (400 MHz, DMSO) δ 11.27 (s, |
| | | | | 1H), 8.73 (s, 1H), 8.59 (t, J = 5.6 Hz, |
| | | | | 1H), 8.26-8.04 (m, 2H), 7.79-7.63 |
| | | | | (m, 2H), 7.62-7.40 (m, 4H), 7.40- |
| | | | | 7.20 (m, 4H), 7.14 (d, J = 7.4 Hz, 1H), |
| | | | | 7.01 (s, 1H), 6.22 (d, J = 6.7 Hz, 2H), |
| | | | | 4.73 (t, J = 6.9 Hz, 1H), 4.33 (d, J = 5.5 |
| | | | | Hz, 2H), 1.60 (d, J = 6.7 Hz, 3H). |
| | | | | LC/MS (ESI) m/z: 605(M + H)⁺. RT |
| | | | | (Method A): 1.78 min. |
| | | | 396 | ¹H NMR (400 MHz, DMSO) 0 11.31 (s, |
| | | | | 1H), 8.61 (t, J = 5.7 Hz, 1H), 8.27 (d, J = |
| | | | | 7.4 Hz, 1H), 8.05 (d, J = 7.6 Hz, 1H), |
| | | | | 7.71 (dd, J = 9.4, 4.5 Hz, 2H), 7.51 (t, J = |
| | | | | 7.8 Hz, 2H), 7.44-7.22 (m, 5H), |
| | | | | 7.14 (s, 1H), 6.97 (s, 1H), 6.23 (s, 1H), |
| | | | | 6.02 (d, J = 7.5 Hz, 1H), 4.88 (t, J = 7.0 |
| | | | | Hz, 2H), 4.34 (d, J = 5.5 Hz, 3H), 1.64 |
| | | | | (d, J = 6.7 Hz, 3H). LC/MS (ESI) m/z: |
| | | | | 605 (M + H)⁺. RT (Method A): 1.79 min. |

### Scheme 86. Synthesis of N-((1H-pyrrolo[3,2-c]pyridine-2-yl)methyl)-2-(6-oxo-2-phenyl-5-(((5-phenylthiophen-2-yl)methyl)amino)pyrimidin-1(6H)-yl)acetamide (Compound 258)

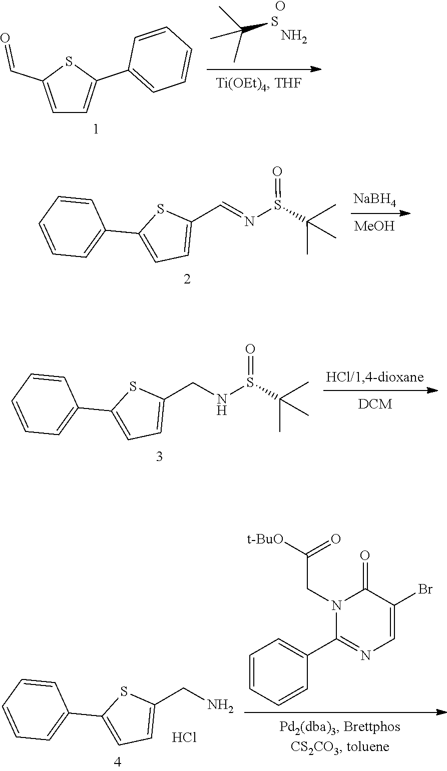

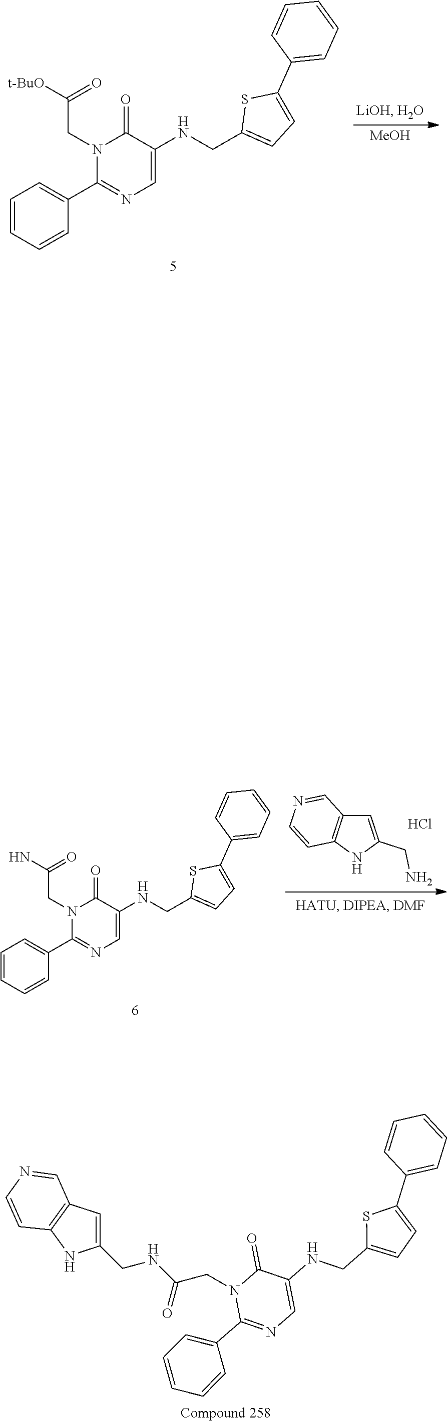

### Step 1: (R,E)-2-methyl-N-((5-phenylthiophen-2-yl)methylene)propane-2-sulfinamide (2)

To a mixture of 5-phenylthiophene-2-carbaldehyde (500 mg, 2.65 mmol) and (R)-2-methylpropane-2-sulfinamide (843 mg, 3.98 mmol) in THF (10 mL) was added Ti(OEt)₄ (1.21 g, 5.30 mmol), the mixture was stirred at 70° C. for 6 hours. The mixture was quenched with ice-water and filtered. The filtrate was extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give the title compound (700 mg, yield 90.8%) as a yellow solid. LC/MS (ESI) m/z: 292 (M+H)⁺.

### Step 2: (R)-2-Methyl-N-((5-phenylthiophen-2-yl)methyl)propane-2-sulfinamide (3)

To a solution of (R,E)-2-methyl-N-((5-phenylthiophen-2-yl)methylene)propane-2-sulfinamide (1.10 g, 3.78 mmol) in MeOH (15 mL) was added NaBH₄ (265 mg, 6.97 mmol) at 0° C. and the reaction mixture was stirred at 25° C. for 1 hour. The mixture was quenched with 1 N aq. HCl and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (667 mg, yield 60.6%) as a white solid. LC/MS (ESI) m/z: 294 (M+H)⁺.

### Step 3: (5-Phenylthiophen-2-yl)methanamine hydrochloride (4)

To a solution of (R)-2-methyl-N-((5-phenylthiophen-2-yl)methyl)propane-2-sulfinamide (667 mg, 2.29 mmol) in DCM (2 mL) was added HCl/1,4-dioxane (4 mL, 4M), the mixture was stirred under N₂ atmosphere at room temperature for 2 hours. The reaction mixture was concentrated under reduced pressure to dryness to give the title compound (500 mg, yield 97.4%) as a white solid, which was used directly in the next step without further purification. LC/MS (ESI) m/z: 190 (M+H)⁺.

### Step 4: Tert-butyl 2-(6-oxo-2-phenyl-5-(((5-phenylthiophen-2-yl)methyl)amino) pyrimidin-1(6H)-yl)acetate (5)

To a mixture of (5-phenylthiophen-2-yl)methanamine hydrochloride (240 mg, 1.06 mmol) and tert-butyl 2-(5-bromo-6-oxo-2-phenylpyrimidin-1(6H)-yl)acetate (389 mg, 1.06 mmol) in toluene (8 mL) was added Pd₂(dba)₃ (97 mg, 0.10 mmol), Cs₂CO₃ (739 mg, 3.20 mmol) and Brettphos (57 mg, 0.10 mmol) under N₂ atmosphere, the reaction mixture was degassed under N₂ atmosphere for three times and stirred under N₂ atmosphere at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give the title compound (300 mg, yield 59.4%) as a white solid. LC/MS (ESI) m/z: 474 (M+H)⁺.

### Step 5: 2-(6-Oxo-2-phenyl-5-(((5-phenylthiophen-2-yl)methyl)amino)pyrimidin-1(6H)-yl)acetic acid (6)

To a solution of tert-butyl 2-(6-oxo-2-phenyl-5-(((5-phenylthiophen-2-yl)methyl)amino) pyrimidin-1(6H)-yl)acetate (300 mg, 0.63 mmol) in MeOH (3 mL) and water (1 mL) was added LiOH (80 mg, 1.90 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (250 mg, yield 94.6%) as a white solid, which was used directly in the next step without further purification. LC/MS (ESI) m/z: 418 (M+H)⁺.

### Step 6: N-(1-(2-(((1H-pyrrolo[3,2-c]pyridine-2-yl)methyl)amino)-2-oxoethyl)-6-oxo-2-phenyl-1,6-dihydropyrimidin-5-yl)-4-phenylcyclohexane-1-carboxamide (Compound 258)

To a mixture of 2-(6-oxo-2-phenyl-5-(((5-phenylthiophen-2-yl)methyl)amino) pyrimidin-1(6H)-yl)acetic acid (350 mg, 0.839 mmol) and (1H-pyrrolo[3,2-c]pyridine-2-yl) methanamine hydrochloride (230 mg, 1.25 mmol) in DMF (4 mL) was added HATU (383 mg, 1.00 mol), DIPEA (90 mg, 2.51 mmol) and the mixture was stirred at room temperature overnight. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-8% MeOH in DCM) and further purified by prep-HPLC to give Compound 258 (22.0 mg, yield 4.4%) as a white solid. ¹H NMR (400 MHz, DMSO-d₆) δ 11.32 (s, 1H), 8.74 (s, 1H), 8.69 (t, J=5.4 Hz, 1H), 8.12 (d, J=5.7 Hz, 1H), 7.61 (s, 1H), 7.59 (s, 1H), 7.45 (s, 1H), 7.44 (s, 2H), 7.39 (d, J=6.7 Hz, 2H), 7.37 (d, J=2.8 Hz, 3H), 7.33 (d, J=5.5 Hz, 1H), 7.27 (t, J=7.4 Hz, 1H), 7.22 (s, 1H), 7.12 (d, J=3.4 Hz, 1H), 6.31 (s, 1H), 6.26 (t, J=6.3 Hz, 1H), 4.55 (d, J=6.1 Hz, 2H), 4.50 (s, 2H), 4.42 (d, J=5.3 Hz, 2H). LC/MS (ESI) m/z: 547 (M+H)⁺. RT (Method A): 1.60 min.

The following compounds were prepared based on Scheme 86:

| # | Reactant A | Reactant B | Reactant C | Reactant D |
|---|---|---|---|---|
| 259 | 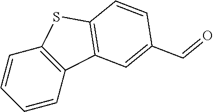 [SMILES: O=Cc1ccc2sc3ccccc3c2c1] | 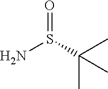 | 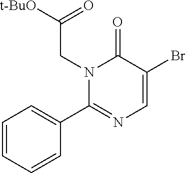 [SMILES: CC(C)(C)OC(=O)Cn1c(-c2ccccc2)ncc(Br)c1=O] | 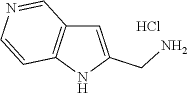 [SMILES: NCc1cc2cnccc2[nH]1 ; Cl] |
| 275^{a} | 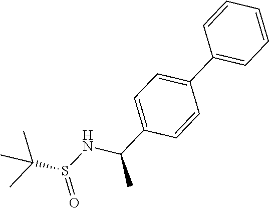 [SMILES: C[C@@H](N[S@](=O)C(C)(C)C)c1ccc(-c2ccccc2)cc1] | | 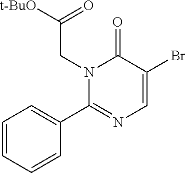 [SMILES: CC(C)(C)OC(=O)Cn1c(-c2ccccc2)ncc(Br)c1=O] | 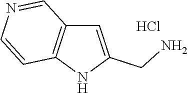 [SMILES: NCc1cc2cnccc2[nH]1 ; Cl] |
| 276^{f} | 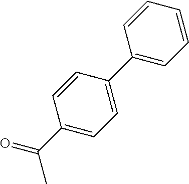 | 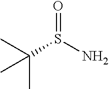 | 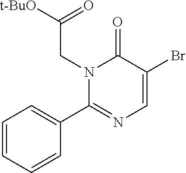 [SMILES: CC(C)(C)OC(=O)Cn1c(-c2ccccc2)ncc(Br)c1=O] | 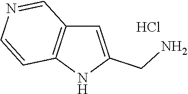 [SMILES: NCc1cc2cnccc2[nH]1 ; Cl] |
| 284^{b} | 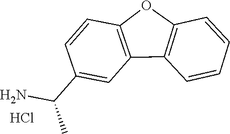 [SMILES: Cl ; C[C@H](N)c1ccc2oc3ccccc3c2c1] | | 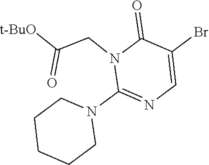 [SMILES: CC(C)(C)OC(=O)Cn1c(N2CCCCC2)ncc(Br)c1=O] | 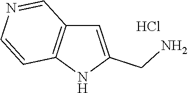 [SMILES: NCc1cc2cnccc2[nH]1 ; Cl] |
| 295^{b} | 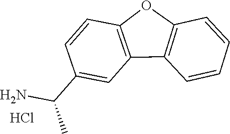 [SMILES: Cl ; C[C@H](N)c1ccc2oc3ccccc3c2c1] | | 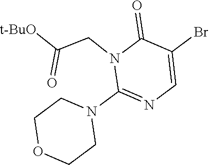 [SMILES: CC(C)(C)OC(=O)Cn1c(N2CCOCC2)ncc(Br)c1=O] | 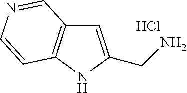 [SMILES: NCc1cc2cnccc2[nH]1 ; Cl] |
| 301^{b,c,d} | 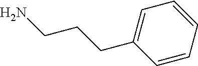 | | 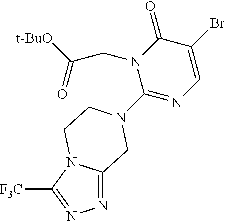 [SMILES: CC(C)(C)OC(=O)Cn1c(N2CCn3c(nnc3C(F)(F)F)C2)ncc(Br)c1=O] | 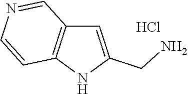 [SMILES: NCc1cc2cnccc2[nH]1 ; Cl] |
| 344^{b} | 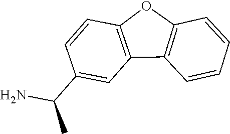 [SMILES: C[C@@H](N)c1ccc2oc3ccccc3c2c1] | | 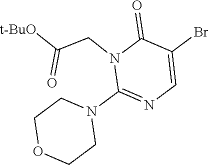 [SMILES: CC(C)(C)OC(=O)Cn1c(N2CCOCC2)ncc(Br)c1=O] | 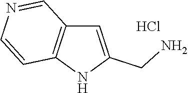 [SMILES: NCc1cc2cnccc2[nH]1 ; Cl] |
| 361^{b} | 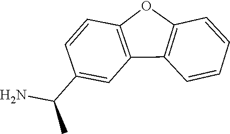 [SMILES: C[C@@H](N)c1ccc2oc3ccccc3c2c1] | | 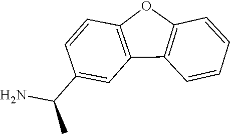 [SMILES: C[C@@H](N)c1ccc2oc3ccccc3c2c1] | 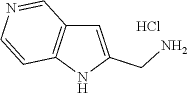 [SMILES: NCc1cc2cnccc2[nH]1 ; Cl] |
| 362^{b,c,d} | 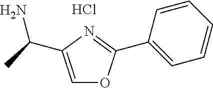 | | 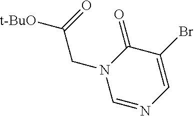 [SMILES: CC(C)(C)OC(=O)Cn1cncc(Br)c1=O] | 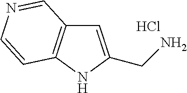 [SMILES: NCc1cc2cnccc2[nH]1 ; Cl] |
| 416^{b,c,d} | 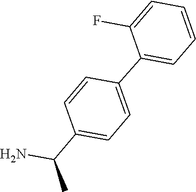 [SMILES: C[C@@H](N)c1ccc(-c2ccccc2F)cc1] | | 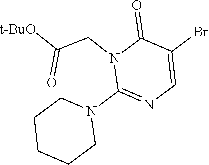 [SMILES: CC(C)(C)OC(=O)Cn1c(N2CCCCC2)ncc(Br)c1=O] | 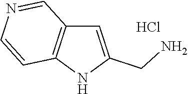 [SMILES: NCc1cc2cnccc2[nH]1 ; Cl] |
| 441^{b} | 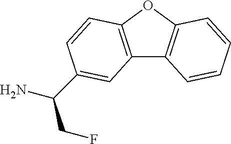 [SMILES: N[C@H](CF)c1ccc2oc3ccccc3c2c1] | | 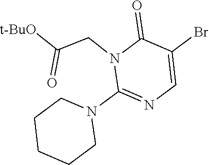 [SMILES: CC(C)(C)OC(=O)Cn1c(N2CCCCC2)ncc(Br)c1=O] | 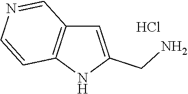 [SMILES: NCc1cc2cnccc2[nH]1 ; Cl] |
| 443^{c,c} | 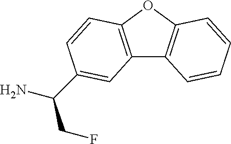 [SMILES: N[C@H](CF)c1ccc2oc3ccccc3c2c1] | | 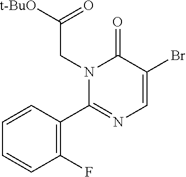 [SMILES: CC(C)(C)OC(=O)Cn1c(-c2ccccc2F)ncc(Br)c1=O] | 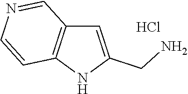 [SMILES: NCc1cc2cnccc2[nH]1 ; Cl] |
| 448^{d,e,f} | 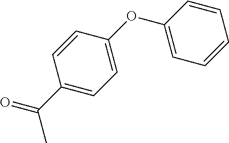 [SMILES: CC(=O)c1ccc(Oc2ccccc2)cc1] | 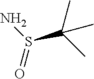 | 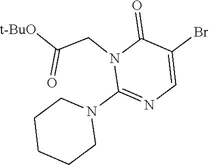 [SMILES: CC(C)(C)OC(=O)Cn1c(N2CCCCC2)ncc(Br)c1=O] | 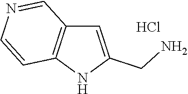 [SMILES: NCc1cc2cnccc2[nH]1 ; Cl] |

| | | | |
|---|---|---|---|
| 460^{b,c,f} | 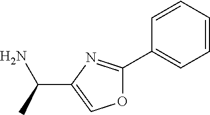 | 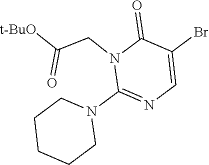 [SMILES: CC(C)(C)OC(=O)Cn1c(N2CCCCC2)ncc(Br)c1=O] | 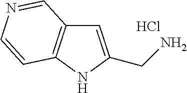 [SMILES: NCc1cc2cnccc2[nH]1 ; Cl] |
| 481^{b,f} | 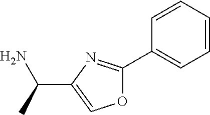 | 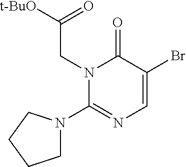 [SMILES: CC(C)(C)OC(=O)Cn1c(N2CCCC2)ncc(Br)c1=O] | 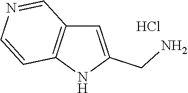 [SMILES: NCc1cc2cnccc2[nH]1 ; Cl] |
| 494^{b} | 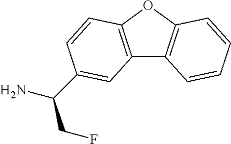 [SMILES: N[C@H](CF)c1ccc2oc3ccccc3c2c1] | 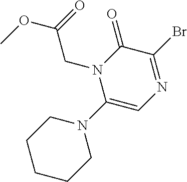 [SMILES: COC(=O)Cn1c(N2CCCCC2)cnc(Br)c1=O] | 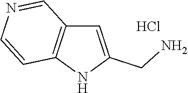 [SMILES: NCc1cc2cnccc2[nH]1 ; Cl] |

| # | Characterization Data |
|---|---|
| 259 | ¹H NMR (400 MHz, DMSO-d₆) δ |
| | 11.36 (s, 1H), 8.76 (s, 1H), 8.70 (t, J = |
| | 5.6 Hz, 1H), 8.41 (s, 1H), 8.33- |
| | 8.30 (m, 1H), 8.15 (d, J = 8.9 Hz, 1H), |
| | 8.04-8.01 (m, 1H), 7.99 (d, J = 8.2 |
| | Hz, 1H), 7.60-7.57 (m, 1H), 7.53- |
| | 7.49 (m, 2H), 7.41 (d, J = 7.2 Hz, 3H), |
| | 7.37-7.33 (m, 3H), 7.14 (s, 1H), |
| | 6.37 (t, J = 6.4 Hz, 1H), 6.34 (s, 1H), |
| | 4.53 (d, J = 6.4 Hz, 2H), 4.50 (s, 2H), |
| | 4.43 (d, J = 5.5 Hz, 2H)). LC/MS |
| | (ESI) m/z: 571 (M + H)⁺. RT (Method |
| | A): 1.71 min. |
| 275^{a} | ¹H NMR (400 MHz, CD₃OD) δ 8.72 |
| | (d, J = 0.8 Hz, 1H), 8.12 (d, J = 5.8 |
| | Hz, 1H), 7.59 (d, J = 5.7 Hz, 2H), |
| | 7.58 (s, 2H), 7.47 (d, J = 8.2 Hz, 2H), |
| | 7.43 (s, 2H), 7.41 (s, 3H), 7.40 (s, |
| | 1H), 7.37-7.33 (m, 2H), 7.31 (t, J = |
| | 6.4 Hz, 1H), 6.90 (s, 1H), 6.49 (s, |
| | 1H), 4.63 (d, J = 2.8 Hz, 2H), 4.59- |
| | 4.56 (m, 1H), 4.55 (s, 2H), 1.63 (d, J = |
| | 6.7 Hz, 3H). LC/MS (ESI) m/z: 555 |
| | (M + H)⁺. RT (Method A): 1.73 min. |
| 276^{f} | ¹H NMR (400 MHz, CD₃OD) δ 8.72 |
| | (d, J = 0.8 Hz, 1H), 8.12 (d, J = 5.9 |
| | Hz, 1H), 7.60 (d, J = 1.1 Hz, 2H), |
| | 7.58 (s, 2H), 7.47 (d, J = 8.2 Hz, 2H), |
| | 7.43 (d, J = 1.0 Hz, 2H), 7.41 (d, J = |
| | 1.7 Hz, 3H), 7.40 (s, 1H), 7.36 (d, J = |
| | 6.6 Hz, 2H), 7.33-7.31 (m, 1H), 6.90 |
| | (s, 1H), 6.49 (d, J = 0.7 Hz, 1H), 4.63 |
| | (d, J = 2.8 Hz, 2H), 4.59-4.56 (m, |
| | 1H), 4.55 (s, 2H), 1.63 (d, J = 6.7 Hz, |
| | 3H). LC/MS (ESI) m/z: 555 (M + H)⁺. |
| | RT (Method A): 1.72 min. |
| 284^{b} | ¹H NMR (400 MHz, CD₃OD) δ 8.71 |
| | (s, 1H), 8.10 (d, J = 5.8 Hz, 1H), 8.04 |
| | (s, 1H), 7.98 (d, J = 7.8 Hz, 1H), 7.57 |
| | - 7.49 (m, 3H), 7.46 (t, J = 7.9 Hz, |
| | 1H), 7.40 (d, J = 5.9 Hz, 1H), 7.33 (t, |
| | J = 7.5 Hz, 1H), 6.71 (s, 1H), 6.58 (s, |
| | 1H), 4.82 (s, 2H), 4.61 (s, 2H), 4.58 |
| | (dd, J= 6.9, 0.4 Hz, 1H), 2.80 (s, 4H), |
| | 1.63 (d, J = 6.6 Hz, 3H), 1.53-1.44 |
| | (m, 6H). LC/MS (ESI) m/z: 576 |
| | (M + H)⁺. RT (Method A): 1.82 min. |
| 295^{b} | ¹H NMR (400 MHz, CH₃OD) δ 8.73 |
| | (s, 1H), 8.11 (d, J = 5.2 Hz, 1H), 8.04 |
| | (s, 1H), 7.98 (d, J = 7.8 Hz, 1H), 7.57- |
| | 7.50 (m, 3H), 7.46-7.41 (m, 2H), |
| | 7.32 (d, J = 7.4 Hz, 1H), 6.74 (s, 1H), |
| | 6.60 (s, 1H), 4.61 (s, 4H), 3.57 (s, |
| | 5H), 2.85 (s, 4H), 1.63 (d, J = 6.1 Hz, |
| | 3H). LC/MS (ESI) m/z: 578 (M + H)⁺. |
| | RT (Method A): 1.58 min. |
| 301^{b,c,d} | ¹H NMR (400 MHz, DMSO-d₆) δ |
| | 11.39 (s, 1H), 8.84 (t, J = 5.6 Hz, 1H), |
| | 8.74 (s, 1H), 8.13 (d, J = 5.9 Hz, 1H), |
| | 7.35 (d, J = 5.8 Hz, 1H), 7.27 (d, J = |
| | 7.2 Hz, 2H), 7.22 (d, J = 7.5 Hz, 2H), |
| | 7.17 (t, J = 6.9 Hz, 1H), 6.89 (s, 1H), |
| | 6.40 (s, 1H), 4.77 (s, 2H), 4.46 (d, J = |
| | 4.9 Hz, 2H), 4.41 (s, 2H), 4.17 (t, J = |
| | 4.5 Hz, 2H), 3.49 (t, J = 4.6 Hz, 2H), |
| | 3.03 (t, J = 6.7 Hz, 2H), 2.66 (d, J = |
| | 5.9 Hz, 1H), 2.63 (d, J = 7.7 Hz, 1H), |
| | 1.88-1.82 (m, 2H). LC/MS (ESI) |
| | m/z: 607 (M + H)⁺. RT (Method A): |
| | 1.37 min. |
| 344^{b} | ¹H NMR (400 MHz, DMSO-d₆) δ |
| | 11.30 (s, 1H), 8.82-8.74 (m, 1H), |
| | 8.74 (s, 1H), 8.18 (s, 1H), 8.12-8.08 |
| | (m, 2H), 7.69-7.63 (m, 2H), 7.59- |
| | 7.57 (m, 1H), 7.53-7.49 (m, 1H), |
| | 7.41-7.37 (m, 1H), 7.33-7.32 (m, |
| | 1H), 6.75 (s, 1H), 6.44 (s, 1H), 5.53 |
| | (d, J = 6.1 Hz, 1H), 4.74-4.65 (m, |
| | 2H), 4.59-4.52 (m, 1H), 4.46 (s, |
| | 2H), 3.50 (s, 4H), 2.75 (s, 4H), 1.56 |
| | (d, J = 5.8 Hz, 3H). LC/MS (ESI) m/z: |
| | 578 (M + H)⁺. RT (Method A): 1.55 |
| | min. |
| 361^{b} | ¹H NMR (400 MHz, DMSO-d₆) δ |
| | 11.32 (s, 1H), 8.77 (m, 1H), 8.73 (s, |
| | 1H), 8.18 (s, 1H), 8.11 (s, 2H), 7.70- |
| | 7.62 (m, 2H), 7.60-7.55 (m, 1H), |
| | 7.51 (t, J = 8.0 Hz, 1H), 7.39 (t, J = |
| | 7.1 Hz, 1H), 7.34-7.30 (m, 1H), 6.73 |
| | (s, 1H), 6.43 (s, 1H), 5.44 (d, J = 5.1 |
| | Hz, 1H), 4.70-4.60 (m, 2H), 4.57- |
| | 4.50 (m, 1H), 4.47 (s, 2H), 2.71 (s, |
| | 4H), 1.56 (d, J = 5.3 Hz, 3H), 1.48- |
| | 1.41 (m, 4H), 1.40-1.32 (m, 2H). |
| | LC/MS (ESI) m/z: 576 (M + H)⁺. RT |
| | (Method A): 1.83 min. |
| 362^{b,c,d} | ¹H NMR (400 MHz, CD₃OD) δ 8.81 |
| | (s, 1H), 8.15 (d, J = 6.2 Hz, 1H), 8.01- |
| | 7.98 (m, 2H), 7.85 (s, 1H), 7.78 (s, |
| | 1H), 7.55 (d, J = 6.1 Hz, 1H), 7.50- |
| | 7.47 (m, 3H), 7.15 (s, 1H), 6.71 (s, |
| | 1H), 4.74 (s, 2H), 4.64 (s, 2H), 4.62- |
| | 4.57 (m, 1H), 1.63 (d, J = 6.7 Hz, |
| | 3H). LC/MS (ESI) m/z: 470 (M + H)⁺. |
| | RT (Method A): 1.01 min. |
| 416^{b,c,d} | ¹H NMR (400 MHz, DMSO-d₆) δ |
| | 11.33 (s, 1H), 8.78 (t, J = 5.7 Hz, 1H), |
| | 8.73 (s, 1H), 8.11 (d, J = 5.7 Hz, 1H), |
| | 7.54 (dd, J = 7.9, 1.8 Hz, 1H), 7.51- |
| | 7.48 (s, 4H), 7.41-7.38 (m, 1H), |
| | 7.32 (d, J = 5.6 Hz, 1H), 7.30-7.26 |
| | (m, 2H), 6.68 (s, 1H), 6.43 (d, J = 0.8 |
| | Hz, 1H), 5.41 (d, J = 7.0 Hz, 1H), |
| | 4.66 (d, J = 3.8 Hz, 2H), 4.47 (d, J = |
| | 5.6 Hz, 2H), 4.45-4.41 (m, 1H), 2.77- |
| | 2.73 (m, 4H), 1.50 (d, J = 6.8 Hz, |
| | 3H), 1.47-1.39 (m, 6H). LC/MS |
| | (ESI) m/z: 580 (M + H)⁺. RT (Method |
| | A): 1.82 min. |
| 441^{b} | ¹H NMR (400 MHz, DMSO-d₆) δ |
| | 11.33 (s, 1H), 8.79 (t, J = 5.6 Hz, 1H), |
| | 8.74 (s, 1H), 8.25 (d, J = 1.4 Hz, 1H), |
| | 8.11 (d, J = 5.7 Hz, 1H), 8.08 (d, J = |
| | 7.7 Hz, 1H), 7.71-7.68 (m, 2H), 7.65 |
| | - 7.63 (m, 1H), 7.54-7.50 (m, 1H), |
| | 7.41 (t, J = 7.6 Hz, 1H), 7.33 (d, J = |
| | 5.7 Hz, 1H), 6.85 (s, 1H), 6.44 (s, |
| | 1H), 5.76 (d, J = 6.9 Hz, 1H), 4.90- |
| | 4.80 (m, 2H), 4.76-4.72 (m, 1H), |
| | 4.66 (d, J = 5.2 Hz, 2H), 4.47 (d, J = |
| | 5.6 Hz, 2H), 2.75-2.71 (m, 4H), 1.47- |
| | 1.39 (m, 6H). LC/MS (ESI) (m/z): |
| | 594 (M + H)⁺. RT (Method A): 1.78 |
| | min. |
| 443^{c,c} | ¹H NMR (400 MHz, DMSO-d₆) δ |
| | 11.26 (s, 1H), 8.73 (s, 1H), 8.60 (t, J = |
| | 5.5 Hz, 1H), 8.31 (s, 1H), 8.12- |
| | 8.07 (m, 2H), 7.72-7.67 (m, 3H), |
| | 7.55-7.47 (m, 2H), 7.43-7.39 (m, |
| | 1H), 7.36-7.26 (m, 3H), 7.17-7.11 |
| | (m, 2H), 6.38 (d, J = 7.6 Hz, 1H), 6.22 |
| | (s, 1H), 5.07-4.86 (m, 2H), 4.86- |
| | 4.64 (m, 2H), 4.45-4.10 (m, 3H). |
| | LC/MS (ESI) m/z: 605 (M + H)⁺. RT |
| | (Method A: 1.66 min. |
| 448^{d,e,f} | ¹H NMR (400 MHz, DMSO-d₆) δ |
| | 11.35 (s, 1H), 8.78 (t, J = 5.6 Hz, 1H), |
| | 8.74 (s, 1H), 8.11 (d, J = 5.7 Hz, 1H), |
| | 7.42-7.35 (m, 4H), 7.33 (d, J = 5.6 |
| | Hz, 1H), 7.13 (t, J = 7.4 Hz, 1H), 7.00 |
| | (d, J = 7.8 Hz, 2H), 6.94 (d, J = 8.6 |
| | Hz, 2H), 6.66 (s, 1H), 6.44 (s, 1H), |
| | 5.32 (d, J = 6.9 Hz, 1H), 4.71-4.59 |
| | (m, 2H), 4.47 (d, J = 5.6 Hz, 2H), 4.40- |
| | 4.31 (m, 1H), 2.82-2.71 (m, 4H), |
| | 1.51-1.46 (m, 4H), 1.45 (d, J = 6.7 |
| | Hz, 3H), 1.44-1.37 (m, 2H). LC/MS |
| | (ESI) m/z: 578 (M + H)⁺. RT (Method |
| | A): 1.85 min. |
| 460^{b,c,f} | ¹H NMR (400 MHz, CD₃OD) δ 8.70 |
| | (s, 1H), 8.09 (d, J = 5.8 Hz, 1H), 8.03- |
| | 7.97 (m, 2H), 7.83 (s, 1H), 7.51- |
| | 7.46 (m, 3H), 7.38 (d, J = 5.8 Hz, 1H), |
| | 7.01 (s, 1H), 6.56 (s, 1H), 4.82 (s, |
| | 2H), 4.60 (s, 2H), 4.57-4.51 (m, |
| | 1H), 2.89 (s, 4H), 1.62 (d, J = 6.7 Hz, |
| | 3H), 1.59-1.47 (m, 6H). LC/MS |
| | (ESI) m/z: 553 (M + H)⁺. RT (Method |
| | A): 1.47 min. |
| 481^{b,f} | ¹H NMR (400 MHz, CD₃OD) δ 8.69 |
| | (s, 1H), 8.08 (d, J = 5.8 Hz, 1H), 8.01- |
| | 7.97 (m, 2H), 7.81 (s, 1H), 7.50- |
| | 7.47 (m, 3H), 7.37 (d, J = 5.8 Hz, 1H), |
| | 7.02 (s, 1H), 6.55 (s, 1H), 4.76 (s, |
| | 2H), 4.60 (s, 2H), 4.53-4.48 (m, |
| | 1H), 3.26-3.22 (m, 4H), 1.82-1.78 |
| | (m, 4H), 1.61 (d, J = 6.7 Hz, 3H). |
| | LC/MS (ESI) m/z: 539 (M + H)⁺. RT |
| | (Method A): 1.39 min. |
| 494^{b} | ¹H NMR (400 MHz, CD₃OD) 0 8.75 |
| | (s, 1H), 8.48 (s, 1H), 8.16 (d, J = 6.2 |
| | Hz, 1H), 8.11 (s, 1H), 7.97 (d, J = 7.6 |
| | Hz, 1H), 7.58-7.55 (m, 2H), 7.54- |
| | 7.49 (m, 2H), 7.45 (t, J = 7.7 Hz, 1H), |
| | 7.31 (t, J = 7.5 Hz, 1H), 7.10 (s, 1H), |
| | 6.57 (s, 1H), 5.54-5.45 (m, 1H), |
| | 4.94 (d, J = 3.5 Hz, 2H), 4.82 (d, J = |
| | 5.8 Hz, 1H), 4.71-4.67 (m, 3H), 3.11- |
| | 3.06 (m, 4H), 1.52-1.43 (m, 6H). |
| | LC/MS (ESI) (m/z): 594 (M + H)⁺. RT |
| | (Method A): 2.09 min. |

^{a}Steps 3-6 only.
^{b}Steps 4-6 only.
^{c}BINAP was used in place of BrettPhos in Step 4.
^{d}Step 5 was performed in a mixture of MeOH, THF, and H₂O.
^{e}Step 1 was performed in 1,4-dioxane.
^{f}.XantPhos was used in place of BrettPhos in Step 4.
^{f}Step 2 produced a mixture of (R)-N-((S)-1-([1,1′-biphenyl]-4-yl)ethyl)-2-methylpropane-2-sulfinamide and (R)-N-((R)-1-([1,1'-biphenyl]-4-yl)ethyl)-2-methylpropane-2-sulfinamide and (R)-N-((S)-1-([1,1′-biphenyl]-4-yl)ethyl)-2-methylpropane-2-sulfinamide was isolated and used in Step 3.

Compound 185 is prepared based on Steps 4-6 in Scheme 86:

| # | Reactant A | Reactant B | Reactant C |
|---|---|---|---|
| 185 | 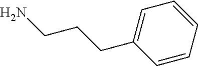 | 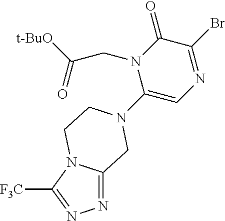 [SMILES: CC(C)(C)OC(=O)Cn1c(N2CCn3c(nnc3C(F)(F)F)C2)cnc(Br)c1=O] | 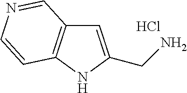 [SMILES: NCc1cc2cnccc2[nH]1 ; Cl] |

### Scheme 87. Synthesis of N-(1-(2-(((1H-pyrrolo[3,2-c]pyridine-2-yl)methyl)amino)-2-oxoethyl)-2-(4-(((3R,3aR,6R,6aR)-6-methoxyhexahydrofuro[3,2-b]furan-3-yl)oxy) dihydropyrimidin-5-yl)-2-phenylthiazole-5-carboxamide(Compound 262)

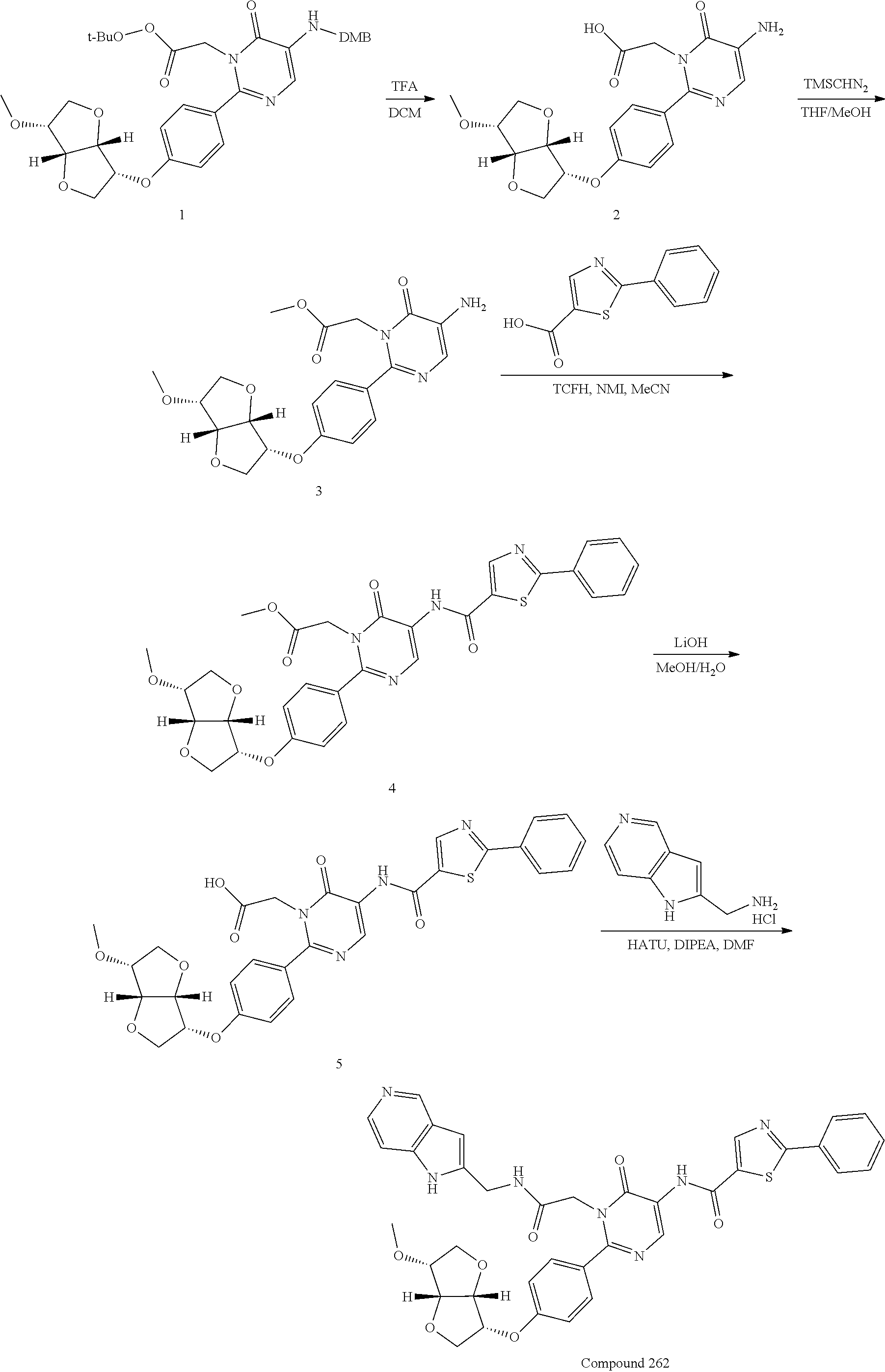

### Step 1: 2-(5-Amino-2-(4-(((3R,3aR,6R,6aR)-6-methoxyhexahydrofuro[3,2-b]furan-3-yl)oxy)phenyl)-6-oxopyrimidin-1(6H)-yl)acetic acid (2)

To a solution of tert-butyl 2-(5-((3,4-dimethylbenzyl)amino)-2-(4-(((3R,3aR,6R,6aR)-6-methoxyhexahydrofuro[3,2-b]furan-3-yl)oxy)phenyl)-6-oxopyrimidin-1(6H)-yl) acetate (101 mg, 0.17 mmol) in DCM (1 mL) was added TFA (1 mL) and the mixture was stirred at room temperature for 2 hours. The reaction mixture was concentrated under reduced pressure to dryness to give the title compound (67 mg, yield 97.6%) as a yellow solid, which was used directly in the next step without further purification. LC/MS (ESI) m/z: 404 (M+H)⁺.

### Step 2: Methyl 2-(5-amino-2-(4-(((3R,3aR,6R,6aR)-6-methoxyhexahydrofuro[3,2-b]furan-3-yl)oxy)phenyl)-6-oxopyrimidin-1(6H)-yl)acetate (3)

To a solution of 2-(5-amino-2-(4-(((3R,3aR,6R,6aR)-6-methoxyhexahydrofuro[3,2-b]furan-3-yl)oxy)phenyl)-6-oxopyrimidin-1(6H)-yl)acetic acid (67 mg, 0.17 mmol) in THF (1 mL) and MeOH (1 mL) was added TMSCHN₂ (0.17 mL, 2M) at 0° C. and the mixture was stirred at room temperature for 3 hours. The mixture was diluted with DCM, washed with saturated aq. NaHCO₃ solution and brine, dried over anhydrous Na₂SO₄, filtered, and concentrated under reduced pressure to dryness. The residue was purified by flash chromatography (silica gel, 0-5% MeOH in DCM) to give the title compound (28 mg, yield 40.6%) as a yellow solid. LC/MS (ESI) m/z: 418 (M+H)⁺.

### Step 3: Methyl 2-(2-(4-(((3R,3aR,6R,6aR)-6-methoxyhexahydrofuro[3,2-b]furan-3-yl)oxy)phenyl)-6-oxo-5-(2-phenylthiazole-5-carboxamido)pyrimidin-1(6H)-yl) acetate (4)

To a mixture of methyl 2-(5-amino-2-(4-(((3R,3aR,6R,6aR)-6-methoxyhexahydrofuro[3,2-b]furan-3-yl)oxy)phenyl)-6-oxopyrimidin-1(6H)-yl) acetate (28 mg, 0.067 mmol) and 2-phenylthiazole-5-carboxylic acid (17 mg, 0.080 mmol) in MeCN (1 mL) was added NMI (16 mg, 0.20 mmol) and TCFH (56 mg, 0.20 mmol) and the mixture was stirred at room temperature for 1 hour. The mixture was diluted with DCM, washed with saturated aq. NaHCO₃ solution and brine, dried over anhydrous Na₂SO₄, filtered, and concentrated under reduced pressure to dryness. The residue was purified by prep-TLC (DCM:MeOH=10:1) to give the title compound (18 mg, yield 44.4%) as a white solid. LC/MS (ESI) m/z: 605 (M+H)⁺.

### Step 4: 2-(2-(4-(((3R,3aR,6R,6aR)-6-methoxyhexahydrofuro[3,2-b]furan-3-yl)oxy) phenyl)-6-oxo-5-(2-phenylthiazole-5-carboxamido)pyrimidin-1(6H)-yl)acetic acid (5)

To a solution of methyl 2-(2-(4-(((3R,3aR,6R,6aR)-6-methoxyhexahydrofuro[3,2-b]furan-3-yl)oxy)phenyl)-6-oxo-5-(2-phenylthiazole-5-carboxamido)pyrimidin-1(6H)-yl)acetate (18 mg, 0.030 mmol) in MeOH/water (1.25 mL, v/v=4/1) was added LiOH (4 mg, 0.090 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (17 mg, yield 95.9%) as a white solid, which was used directly in the next step without further purification. LC/MS (ESI) m/z: 591 (M+H)⁺.

### Step 5: N-(1-(2-(((1H-pyrrolo[3,2-c]pyridine-2-yl)methyl)amino)-2-oxoethyl)-2-(4-(((3R,3aR,6R,6aR)-6-methoxyhexahydrofuro[3,2-b]furan-3-yl)oxy)phenyl)-6-oxo-1,6-dihydropyrimidin-5-yl)-2-phenylthiazole-5-carboxamide (Compound 262)

To a mixture of 2-(2-(4-(((3R,3aR,6R,6aR)-6-methoxyhexahydrofuro[3,2-b]furan-3-yl)oxy)phenyl)-6-oxo-5-(2-phenylthiazole-5-carboxamido)pyrimidin-1(6H)-yl)acetic acid (17 mg, 0.030 mmol) and (1H-pyrrolo[3,2-c]pyridine-2-yl)methanamine hydrochloride (6 mg, 0.037 mmol) in DMF (1 mL) was added DIPEA (19 mg, 0.15 mmol) and HATU (14 mg, 0.037 mmol) and the reaction mixture was stirred at room temperature for 2 hours. The mixture was concentrated under reduced pressure to dryness. The residue was purified by prep-HPLC to give Compound 262 (1.5 mg, yield 6.9%) as a white solid. ¹H NMR (400 MHz, DMSO-d₆) δ 11.36 (s, 1H), 9.99 (s, 1H), 8.83-8.80 (m, 2H), 8.75 (s, 1H), 8.66 (s, 1H), 8.12 (d, J=5.6 Hz, 1H), 8.06-8.03 (m, 2H), 7.58-7.55 (m, 3H), 7.52 (d, J=8.7 Hz, 2H), 7.34 (d, J=5.6 Hz, 1H), 7.04 (d, J=8.8 Hz, 2H), 6.39 (s, 1H), 4.89-4.85 (m, 1H), 4.77 (t, J=5.0 Hz, 1H), 4.60 (t, J=4.7 Hz, 3H), 4.46 (d, J=5.4 Hz, 2H), 4.08-4.04 (m, 1H), 3.93-3.88 (m, 1H), 3.86-3.82 (m, 1H), 3.80-3.76 (m, 1H), 3.46 (t, J=8.2 Hz, 1H), 3.35 (s, 3H). LC/MS (ESI) m/z: 720 (M+H)⁺. RT (Method A): 1.32 min.

### Scheme 89. Synthesis of N-(1-(2-(((1H-pyrrolo[3,2-c]pyridine-2-yl)methyl)amino)-2-oxoethyl)-2-(4-(((3R,3aR,6R,6aR)-6-hydroxyhexahydrofuro[3,2-b]furan-3-yl)oxy)phenyl)-6-oxo-1,6-dihydropyrimidin-5-yl)-2-phenylthiazole-5-carboxamide (Compound 263)

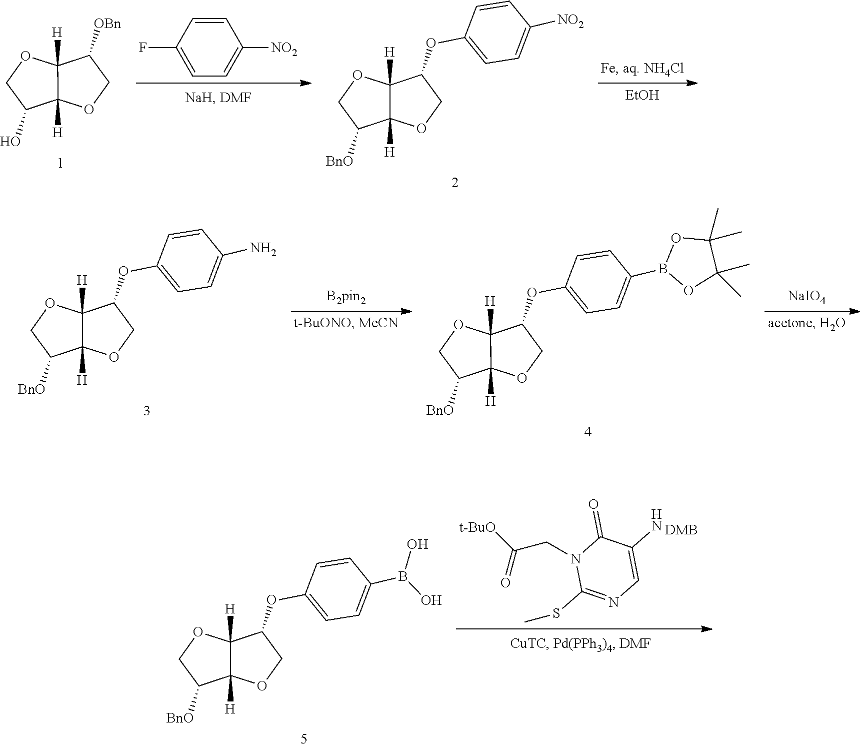

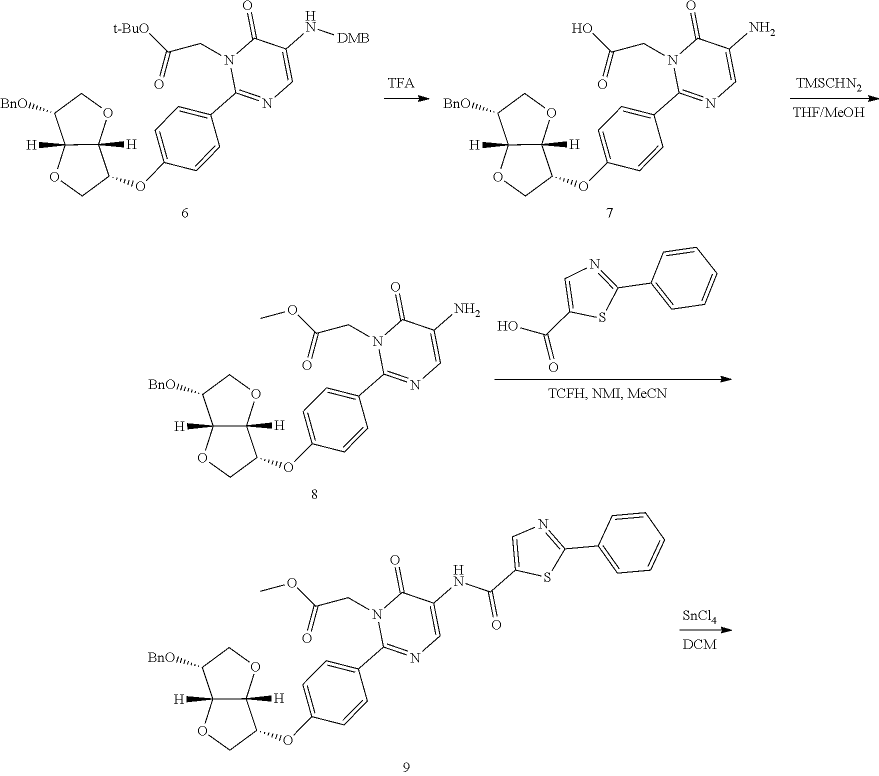

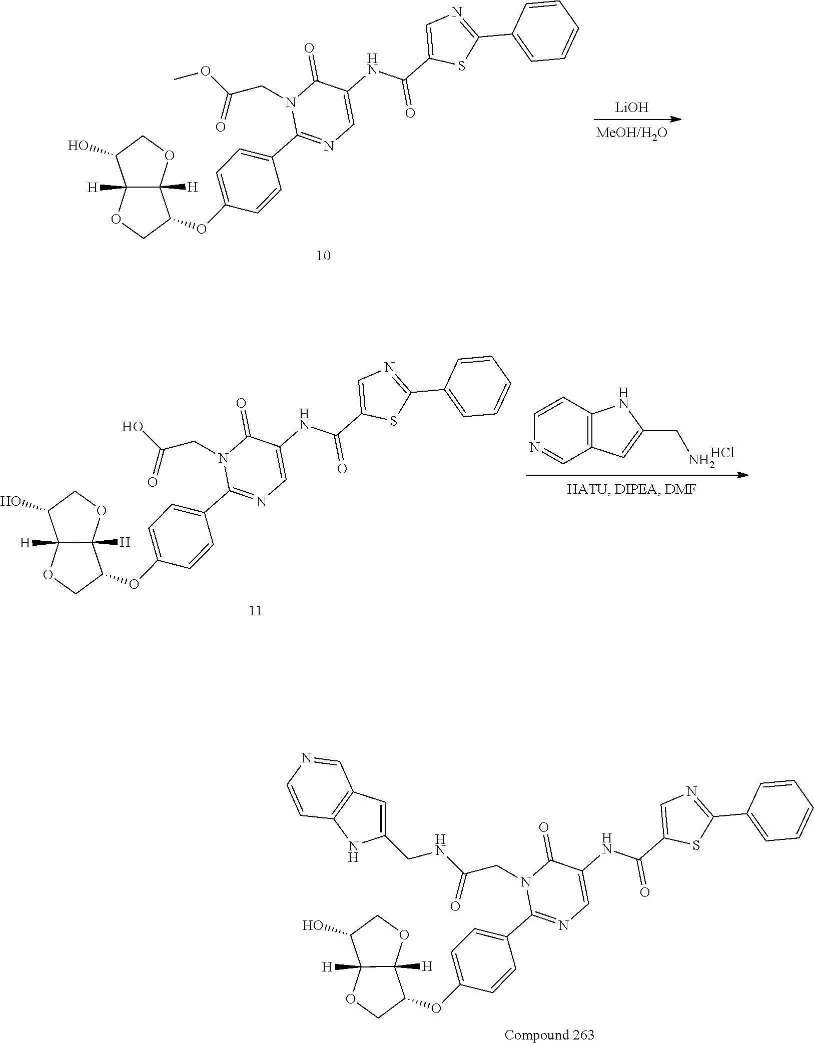

### Step 1: (3R,3aR,6R,6aR)-3-(benzyloxy)-6-(4-nitrophenoxy)hexahydrofuro[3,2-b]furan (2)

To a solution of (3R,3aR,6R,6aR)-6-(benzyloxy)hexahydrofuro[3,2-b]furan-3-ol (2.7 g, 11.43 mmol) in dry DMF (30 mL) was added NaH (330 mg, 13.75 mmol, 60% dispersion in mineral oil) in portions at 0° C. and the mixture was stirred at 0° C. for 0.5 hour. And then, to the reaction mixture was added 1-fluoro-4-nitrobenzene (2.1 g, 14.88 mmol) dropwise at 0° C. and the resulting mixture was stirred at room temperature for 3 hours. The mixture was quenched with saturated aq. NH₄Cl solution and extracted with EtOAc twice. The combined organic layers were washed with brine, dried over anhydrous Na₂SO₄, filtered, and concentrated to dryness. The residue was purified by flash chromatography (silica gel, 0-50% EtOAc in PE) to give the title compound (3.5 g, yield 85.7%) as a yellow oil. LC/MS (ESI) m/z: 358 (M+H)⁺.

### Step 2: 4-(((3R,3aR,6R,6aR)-6-(Benzyloxy)hexahydrofuro[3,2-b]furan-3-yl)oxy) aniline (3)

To a solution of (3R,3aR,6R,6aR)-3-(benzyloxy)-6-(4-nitrophenoxy)hexahydrofuro[3,2-b]furan (3.5 g, 9.79 mmol) in EtOH (35 mL) and saturated aq. NH₄Cl solution (5 mL) was added Fe (1.1 g, 19.64 mmol) under N₂ atmosphere, and the reaction mixture was stirred at 80° C. overnight. The mixture was diluted with EtOAc and filtered. The filtrate was washed with water and brine, dried over anhydrous Na₂SO₄, filtered, and concentrated under reduced pressure to dryness. The residue was purified by flash chromatography (silica gel, 0-100% EtOAc in PE) to give the title compound (3.0 g, yield 93.5%) as a yellow solid. LC/MS (ESI) m/z: 328 (M+H)⁺.

### Step 3: 2-(4-(((3R,3aR,6R,6aR)-6-(Benzyloxy)hexahydrofuro[3,2-b]furan-3-yl)oxy) phenyl)-4,4,5,5-tetramethyl-1,3,2-dioxaborolane (4)

To a solution of 4-(((3R,3aR,6R,6aR)-6-(benzyloxy)hexahydrofuro[3,2-b]furan-3-yl)oxy) aniline (3 g, 9.17 mmol) in MeCN (30 mL) was added t-BuONO (4.7 g, 27.5 mmol) under N₂ atmosphere and the reaction mixture was stirred at 0° C. for 10 minutes. Then 4,4,4′,4′,5,5,5′,5′-octamethyl-2,2′-bi(1,3,2-dioxaborolane) (7.0 g, 45.6 mmol) was added into the above mixture and the resulting mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give the title compound (3.7 g, yield 92.5%) as a yellow oil. LC/MS (ESI) m/z: 439 (M+H)⁺.

### Step 4: (4-(((3R,3aR,6R,6aR)-6-(Benzyloxy)hexahydrofuro[3,2-b]furan-3-yl)oxy) phenyl)boronic acid (5)

To a solution of 2-(4-(((3R,3aR,6R,6aR)-6-(benzyloxy)hexahydrofuro[3,2-b]furan-3-yl)oxy)phenyl)-4,4,5,5-tetramethyl-1,3,2-dioxaborolane (3.7 g, 8.44 mol) in acetone/water (40 mL, v/v=3/1) was added NaIO₄ (9.0 g, 42.0 mmol) at 0° C. and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-20% MeOH in DCM) the title compound (2.05 g, yield 68.2%) as a yellow solid. LC/MS (ESI) m/z: 357 (M+H)⁺.

### Step 5: Tert-butyl 2-(2-(4-(((3R,3aR,6R,6aR)-6-(benzyloxy)hexahydrofuro[3,2-b]furan-3-yl)oxy)phenyl)-5-((3,4-dimethoxybenzyl)amino)-6-oxopyrimidin-1(6H)-yl)acetate (6)

To a mixture of ((4-(((3R,3aR,6R,6aR)-6-(benzyloxy)hexahydrofuro[3,2-b]furan-3-yl)oxy)phenyl)boronic acid (600 mg, 1.68 mmol) and tert-butyl 2-(5-((3,4-dimethoxybenzyl)amino)-2-(methylthio)-6-oxopyrimidin-1(6H)-yl)acetate (708 mg, 3.71 mmol) in DMF (10 mL) was added CuTC (708 mg, 3.71 mmol) and Pd(PPh₃)₄ (390 mg, 0.34 mmol) under N₂ atmosphere, the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-60% EtOAc in PE) to give the title compound (800 mg, yield 69.2%) as a yellow oil. LC/MS (ESI) m/z: 686 (M+H)⁺.

### Step 6: 2-(5-Amino-2-(4-(((3R,3aR,6R,6aR)-6-(benzyloxy)hexahydrofuro[3,2-b]furan-3-yl)oxy)phenyl)-6-oxopyrimidin-1(6H)-yl)acetic acid (7)

A solution of tert-butyl 2-(2-(4-(((3R,3aR,6R,6aR)-6-(benzyloxy)hexahydrofuro[3,2-b]furan-3-yl)oxy)phenyl)-5-((3,4-dimethoxybenzyl)amino)-6-oxopyrimidin-1(6H)-yl) acetate (800 mg, 1.16 mmol) in TFA (8 mL) was stirred room temperature for 1 hour. The reaction mixture was concentrated under reduced pressure to dryness to give the title compound (550 mg, yield 98.2%) as a yellow oil, which was used directly in the next step without further purification. LC/MS (ESI) m/z: 480 (M+H)⁺.

### Step 7: Methyl 2-(5-amino-2-(4-(((3R,3aR,6R,6aR)-6-(benzyloxy)hexahydrofuro[3,2-b]furan-3-yl)oxy)phenyl)-6-oxopyrimidin-1(6H)-yl)acetate (8)

To a solution of 2-(5-amino-2-(4-(((3R,3aR,6R,6aR)-6-(benzyloxy)hexahydrofuro[3,2-b]furan-3-yl)oxy)phenyl)-6-oxopyrimidin-1(6H)-yl)acetic acid (550 mg, 1.14 mol) in THF/MeOH (8 mL, v/v=1/1) was added TMSCHN₂ (5 mL, 2N) at 0° C. under N₂ atmosphere and the mixture was stirred at room temperature for 1 hour. The reaction mixture was quenched with saturated aq. NaHCO₃ solution and extracted with EtOAc twice. The combined organic layers were washed with water and brine, dried over anhydrous Na₂SO₄, filtered, and concentrated under reduced pressure to dryness. The residue was purified by flash chromatography (silica gel, 0-10% MeOH in DCM) to give the title compound (210 mg, yield 37.0%) as a yellow solid. LC/MS (ESI) m/z: 494 (M+H)⁺.

### Step 8: Methyl 2-(2-(4-(((3R,3aR,6R,6aR)-6-(benzyloxy)hexahydrofuro[3,2-b]furan-3-yl)oxy)phenyl)-6-oxo-5-(2-phenylthiazole-5-carboxamido)pyrimidin-1(6H)-yl) acetate (9)

To a mixture of methyl 2-(5-amino-2-(4-(((3R,3aR,6R,6aR)-6-(benzyloxy) hexahydrofuro[3,2-b]furan-3-yl)oxy)phenyl)-6-oxopyrimidin-1(6H)-yl)acetate (50 mg, 0.10 mmol) and 2-phenylthiazole-5-carboxylic acid (31 mg, 0.15 mmol) in MeCN (2 mL) was added NMI (25 mg, 0.30 mmol) and TCFH (85 mg, 0.30 mmol) and the reaction mixture was stirred at room temperature for 1 hour. The mixture was diluted with EtOAc, washed with saturated aq. NaHCO₃ solution, dried over anhydrous Na₂SO₄, filtered, and concentrated under reduced pressure to dryness. The residue was purified by flash chromatography (silica gel, 0-80% EtOAc in PE) to give the title compound (50 mg, yield 72.4%) as a colorless oil. LC/MS (ESI) m/z: 681 (M+H)⁺.

### Step 9: Methyl 2-(2-(4-(((3R,3aR,6R,6aR)-6-hydroxyhexahydrofuro[3,2-b]furan-3-yl)oxy)phenyl)-6-oxo-5-(2-phenylthiazole-5-carboxamido)pyrimidin-1(6H)-yl) acetate (10)

To a solution of methyl 2-(2-(4-(((3R,3aR,6R,6aR)-6-(benzyloxy)hexahydrofuro[3,2-b]furan-3-yl)oxy)phenyl)-6-oxo-5-(2-phenylthiazole-5-carboxamido)pyrimidin-1(6H)-yl)acetate (50 mg, 0.073 mol) in DCM (2 mL) was added SnCl₄ (40 mg, 0.15 mmol) at 0° C. and the reaction mixture was stirred at 50° C. overnight. The mixture was concentrated under reduced pressure to dryness. The residue was purified by flash chromatography (silica gel, 0-10% MeOH in DCM) to give the title compound (30 mg, yield 69.1%) as a yellow solid. LC/MS (ESI) m/z: 591 (M+H)⁺.

### Step 10: 2-(2-(4-(((3R,3aR,6R,6aR)-6-hydroxyhexahydrofuro[3,2-b]furan-3-yl)oxy) phenyl)-6-oxo-5-(2-phenylthiazole-5-carboxamido)pyrimidin-1(6H)-yl)acetic acid (11)

To a solution of methyl 2-(2-(4-(((3R,3aR,6R,6aR)-6-hydroxyhexahydrofuro[3,2-b]furan-3-yl)oxy)phenyl)-6-oxo-5-(2-phenylthiazole-5-carboxamido)pyrimidin-1(6H)-yl)acetate (30 mg, 0.051 mmol) in MeOH/water (2 mL, v/v=4/1) was added LiOH·H₂O (5 mg, 0.12 mmol) and the reaction mixture was stirred at room temperature for 1 hour. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (25 mg, yield 86.2%) as a yellow solid, which was used directly in the next step without further purification. LC/MS (ESI) m/z: 577 (M+H)⁺.

### Step 11: N-(1-(2-(((1H-pyrrolo[3,2-c]pyridine-2-yl)methyl)amino)-2-oxoethyl)-2-(4-(((3R,3aR,6R,6aR)-6-hydroxyhexahydrofuro[3,2-b]furan-3-yl)oxy)phenyl)-6-oxo-1,6-dihydropyrimidin-5-yl)-2-phenylthiazole-5-carboxamide (Compound 263)

To a mixture of 2-(2-(4-(((3R,3aR,6R,6aR)-6-hydroxyhexahydrofuro[3,2-b]furan-3-yl)oxy)phenyl)-6-oxo-5-(2-phenylthiazole-5-carboxamido)pyrimidin-1(6H)-yl)acetic acid (25 mg, 0.043 mmol) and (1H-pyrrolo[3,2-c]pyridine-2-yl)methanamine hydrochloride (10 mg, 0.054 mmol) in DMF (1 mL) was added DIPEA (30 mg, 0.23 mmol) and HATU (20 mg, 0.052 mmol), the mixture was stirred at room temperature for 1 hour. The mixture was concentrated under reduced pressure to dryness. The residue was purified by prep-HPLC to give Compound 263 (5 mg, yield 16.4%) as a yellow solid. ¹H NMR (400 MHz, DMSO-d₆) δ 11.36 (s, 1H), 10.00 (s, 1H), 8.84-8.80 (m, 2H), 8.75 (s, 1H), 8.67 (s, 1H), 8.12 (d, J=5.6 Hz, 1H), 8.06-8.02 (m, 2H), 7.56 (dd, J=5.1, 1.8 Hz, 3H), 7.52 (d, J=8.7 Hz, 2H), 7.34 (d, J=5.6 Hz, 1H), 7.04 (d, J=8.8 Hz, 2H), 6.38 (s, 1H), 4.94 (d, J=6.8 Hz, 1H), 4.87 (dd, J=11.7, 6.3 Hz, 1H), 4.73 (t, J=4.9 Hz, 1H), 4.61 (s, 2H), 4.46 (d, J=5.5 Hz, 2H), 4.37 (t, J=4.9 Hz, 1H), 4.16-4.10 (m, 1H), 4.06 (dd, J=9.1, 6.3 Hz, 1H), 3.80 (dd, J=9.0, 6.5 Hz, 1H), 3.77-3.72 (m, 1H), 3.38 (d, J=8.5 Hz, 1H). LC/MS (ESI) m/z: 706 (M+H)⁺. RT (Method A): 1.22 min.

Compound 264 was prepared based on Scheme 88:

| # | Reactant A | Reactant B | Reactant C | Reactant D |
|---|---|---|---|---|
| 264 | 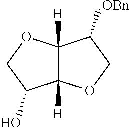 | 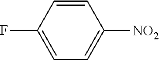 | 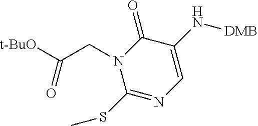 | 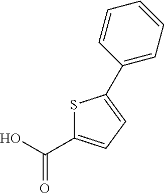 [SMILES: O=C(O)c1ccc(-c2ccccc2)s1] |
| # | | | Reactant E | Characterization Data |
| 264 | | | 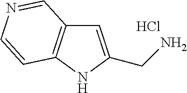 [SMILES: NCc1cc2cnccc2[nH]1 ; Cl] | ¹H NMR (400 MHz, DMSO-d₆) δ 11.36 (s, 1H), 9.65 (s, 1H), 8.82 (t, J = 5.5 Hz, 1H), 8.75 (s, 1H), 8.68 (s, 1H), 8.12 (t, J = 4.6 Hz, 2H), |
| | | | | 7.77 (d, J = 7.4 Hz, 2H), |
| | | | | 7.63 (d, J = 4.0 Hz, 1H), |
| | | | | 7.52-7.46 (m, 4H), |
| | | | | 7.40 (t, J = 7.3 Hz, 1H), |
| | | | | 7.34 (d, J = 5.7 Hz, 1H), |
| | | | | 7.04 (d, J = 8.7 Hz, 2H), |
| | | | | 6.38 (s, 1H), 4.98- |
| | | | | 4.84 (m, 2H), 4.72 (t, J |
| | | | | = 4.9 Hz, 1H), 4.60 (s, |
| | | | | 2H), 4.46 (d, J = 5.4 Hz, |
| | | | | 2H), 4.37 (t, J = 4.8 Hz, |
| | | | | 1H), 4.16-4.10 (m, |
| | | | | 1H), 4.06 (dd, J = 9.0, |
| | | | | 6.4 Hz, 1H), 3.82-3.72 |
| | | | | (m, 2H), 3.41 (s, 1H). |
| | | | | LC/MS (ESI) m/z: 705 |
| | | | | (M + H)⁺. RT (Method |
| | | | | A): 1.40 min. |

### Scheme 89. Synthesis of (S)—N-((1H-pyrrolo[3,2-c]pyridine-2-yl)methyl)-2-(5-((1-(dibenzo[b,d]furan-2-yl)ethyl)amino)-6-oxopyrimidin-1(6H)-yl)acetamide (Compound 265)

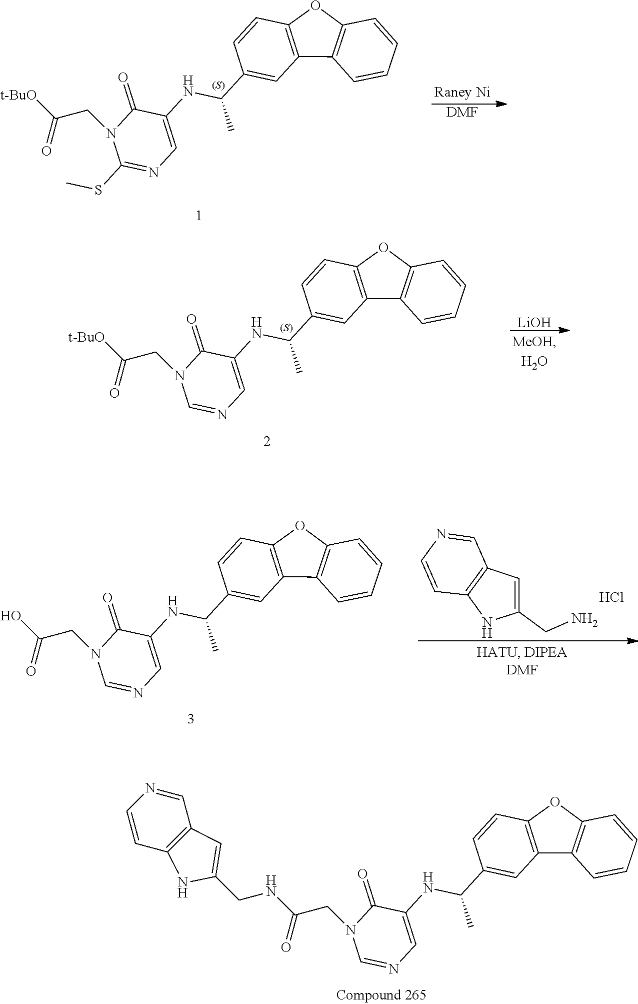

### Step 1: Tert-butyl (S)-2-(5-((1-(dibenzo[b,d]furan-2-yl)ethyl)amino)-6-oxopyrimidin-1(6H)-yl)acetate (2)

To a solution of tert-butyl (S)-2-(5-((1-(dibenzo[b,d]furan-2-yl)ethyl)amino)-2-(methylthio)-6-oxopyrimidin-1(6H)-yl)acetate (290 mg, 0.62 mmol) in DMF (8 mL) was added Raney Ni (200 mg) and the mixture reaction was stirred at 90° C. for 2 hours. The reaction mixture was filtered, and the filtrate was washed with water and brine, dried over anhydrous Na₂SO₄, filtered, and concentrated under reduced pressure to dryness. The residue was purified by flash chromatography (silica gel, 0-18% EtOAc in PE) to give the title compound (250 mg, yield 95.8%) as a light-yellow oil. LC/MS (ESI) m/z: 420 (M+H)⁺.

### Step 2: (S)-2-(5-((1-(dibenzo[b,d]furan-2-yl)ethyl)amino)-6-oxopyrimidin-1(6H)-yl)acetic acid (3)

To a solution of tert-butyl (S)-2-(5-((1-(dibenzo[b,d]furan-2-yl)ethyl)amino)-6-oxopyrimidin-1(6H)-yl)acetate (250 mg, 0.60 mmol) in MeOH/1,4-dioxane/water (8 mL, v/v/v=2/1/1) was added LiOH (57 mg, 2.39 mmol) and the mixture was stirred at 60° C. overnight. The mixture was acidified with 1N aq. HCl to pH˜5 and extracted with EtOAc twice. The combined organic layers were washed with brine, dried over anhydrous Na₂SO₄, filtered, and concentrated under reduced pressure to dryness to give the title compound (215 mg, yield 99.3%) as a white solid, which was used directly in the next step without further purification. LC/MS (ESI) m/z: 364 (M+H)⁺.

### Step 3: (S)—N-((1H-pyrrolo[3,2-c]pyridine-2-yl)methyl)-2-(5-((1-(dibenzo[b,d]furan-2-yl)ethyl)amino)-6-oxopyrimidin-1(6H)-yl)acetamide (Compound 265)

To a mixture of (S)-2-(5-((1-(dibenzo[b,d]furan-2-yl)ethyl)amino)-6-oxopyrimidin-1(6H)-yl)acetic acid (150 mg, 0.41 mmol) and (1H-pyrrolo[3,2-c]pyridine-2-yl)methanamine hydrochloride (151 mg, 0.82 mmol) in DMF (3 mL) was added DIPEA (211 mg, 1.64 mmol) and HATU (156 mg, 0.41 mmol) under N₂ atmosphere and the reaction mixture was stirred at room temperature for 1 hour. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-20% MeOH in DCM) and further purified by prep-HPLC to give Compound 265 (21.9 mg, yield 10.8%) as a white solid. ¹H NMR (400 MHz, CD₃OD) δ 8.71 (s, 1H), 8.10 (d, J=5.9 Hz, 1H), 8.02 (s, 1H), 7.96 (d, J=7.6 Hz, 1H), 7.69 (s, 1H), 7.56-7.43 (m, 4H), 7.39 (d, J=5.8 Hz, 1H), 7.32 (t, J=7.5 Hz, 1H), 6.82 (d, J=9.6 Hz, 1H), 6.57 (s, 1H), 4.72 (s, 2H), 4.61 (s, 2H), 4.59-4.54 (m, 1H), 1.63 (d, J=6.7 Hz, 3H). LC/MS (ESI) m/z: 493 (M+H)⁺. RT (Method A): 1.43 min.

Compound 310 was prepared based on Scheme 89:

| # | Reactant A | Reactant B | Characterization Data |
|---|---|---|---|
| 310 | 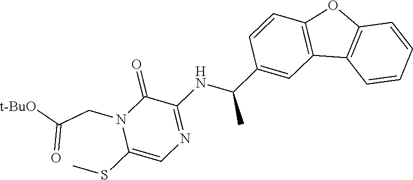 | 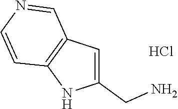 [SMILES: NCc1cc2cnccc2[nH]1 ; Cl] | ¹H NMR (400 MHz, DMSO-d₆) δ 11.41 (s, 1H), 8.85 (s, 1H), 8.76 (s, 1H), 8.18 (s, 1H), 8.15-8.07 (m, 2H), 7.71-7.62 (m, 3H), 7.58 (d, J = 8.4 Hz, 1H), 7.51 (t, J = 7.9 Hz, 1H), 7.41-7.32 (m, 2H), 6.85 (s, 1H), 6.46 (s, 1H), 5.80 (d, J = 6.5 Hz, 1H), 4.69-4.56 (m, 3H), 4.47 (s, 2H), 1.57 (d, J = 6.5 Hz, 3H). LC/MS (ESI) m/z: 493 (M + H)⁺. RT (Method A): 1.41 min. |

### Scheme 90. Synthesis of (S)—N-((1H-pyrrolo[3,2-c]pyridine-2-yl)methyl)-2-(3-((1-(dibenzo[b,d]furan-2-yl)ethyl)amino)-2-oxo-6-phenylpyrazin-1(2H)-yl)acetamide (Compound 282)

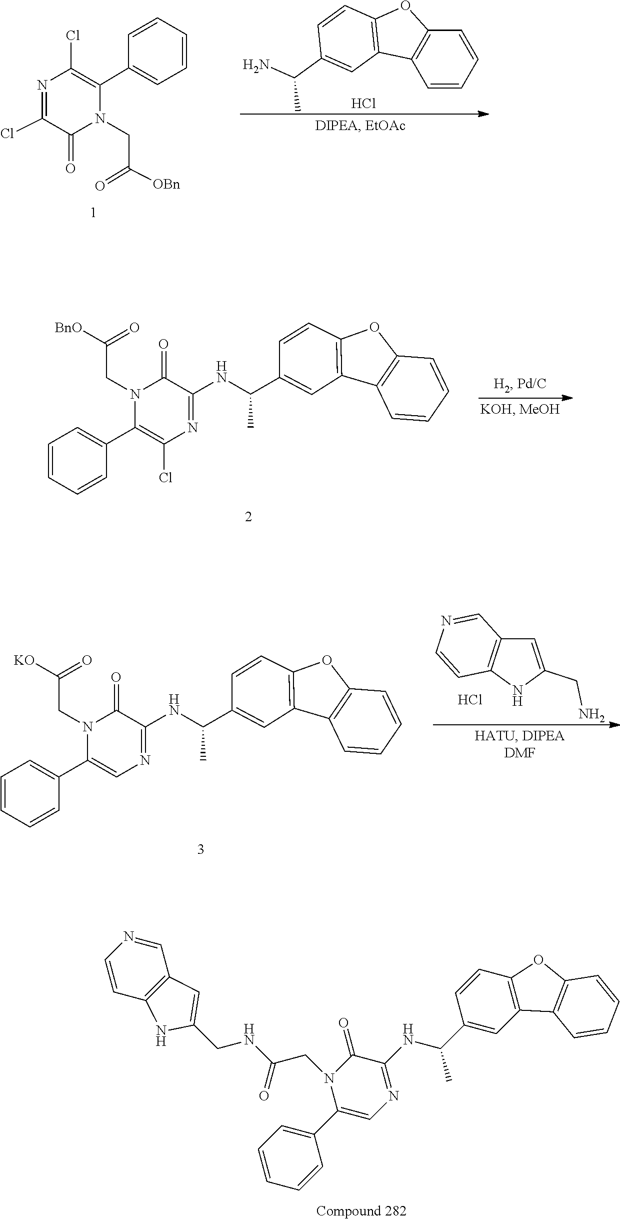

### Step 1: Benzyl (S)-2-(5-chloro-3-((1-(dibenzo[b,d]furan-2-yl)ethyl)amino)-2-oxo-6-phenylpyrazin-1(2H)-yl)acetate (2)

To a mixture of benzyl 2-(3,5-dichloro-2-oxo-6-phenylpyrazin-1(2H)-yl)acetate (500 mg, 1.29 mmol) and (S)-1-(dibenzo[b,d]furan-2-yl)ethan-1-amine hydrochloride (318 mg, 1.29 mmol) in EtOAc (3 mL) was added DIPEA (831 mg, 6.44 mmol) and the mixture was stirred at 80° C. overnight. The mixture was concentrated under reduced pressure to dryness. The residue was purified by flash chromatography (silica gel, 0-30% EtOAc in PE) to give the title compound (570 mg, yield 78.6%) as a yellow solid. LC/MS (ESI) m/z: 564 (M+H)⁺.

### Step 2: Potassium (S)-2-(3-((1-(dibenzo[b,d]furan-2-yl)ethyl)amino)-2-oxo-6-phenylpyrazin-1(2H)-yl)acetate (3)

To a solution of benzyl (S)-2-(5-chloro-3-((1-(dibenzo[b,d]furan-2-yl)ethyl)amino)-2-oxo-6-phenylpyrazin-1(2H)-yl)acetate (250 mg, 0.44 mmol) in MeOH (5 mL) was added KOH (25 mg, 0.44 mmol) and Pd/C (30 mg, 10% wt.), the mixture was degassed under N₂ atmosphere for three times and stirred under a H₂ balloon at 50° C. for 5 hours. The mixture was filtered, and the filtrate was concentrated to dryness to give the title compound (210 mg, yield 100%) as a yellow solid, which was used directly in the next step without further purification. LC/MS (ESI) m/z: 440 (M+H)⁺.

### Step 3: (S)—N-((1H-pyrrolo[3,2-c]pyridine-2-yl)methyl)-2-(3-((1-(dibenzo[b,d]furan-2-yl)ethyl)amino)-2-oxo-6-phenylpyrazin-1(2H)-yl)acetamide (Compound 282)

To a mixture of potassium (S)-2-(3-((1-(dibenzo[b,d]furan-2-yl)ethyl)amino)-2-oxo-6-phenylpyrazin-1(2H)-yl)acetate (196 mg, 0.41 mmol) and (1H-pyrrolo[3,2-c]pyridine-2-yl)methanamine hydrochloride (90 mg, 0.49 mmol) in DMF (3 mL) was added HATU (234 mg, 0.62 mmol) and DIPEA (264 mg, 2.05 mmol) under N₂ atmosphere and the reaction mixture was stirred at room temperature for 1 hour. The mixture was concentrated under reduced pressure to dryness. The residue was purified by flash chromatography (silica gel, 0-3% MeOH in DCM) and further purified by prep-HPLC to give Compound 282 (25 mg, yield 10.7%) as a white solid. ¹H NMR (400 MHz, CD₃OD) δ 8.71 (d, J=0.8 Hz, 1H), 8.10 (d, J=5.9 Hz, 2H), 8.01 (d, J=7.7 Hz, 1H), 7.59-7.52 (m, 3H), 7.48-7.44 (m, 1H), 7.39 (d, J=5.9 Hz, 1H), 7.37-7.35 (m, 1H), 7.34 (d, J=2.8 Hz, 1H), 7.32 (d, J=2.2 Hz, 3H), 7.30 (s, 1H), 6.69 (d, J=3.5 Hz, 1H), 6.47 (s, 1H), 5.36-5.30 (m, 1H), 4.53 (d, J=3.1 Hz, 4H), 1.70 (d, J=6.9 Hz, 3H). LC/MS (ESI) m/z: 569 (M+H)⁺. RT (Method A): 1.83 min.

Compound 322 was prepared based on Scheme 90:

| # | Reactant A | Reactant B | Reactant C | Characterization Data |
|---|---|---|---|---|
| 322 | 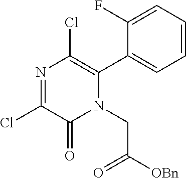 [SMILES: O=C(Cn1c(-c2ccccc2F)c(Cl)nc(Cl)c1=O)OCc1ccccc1] | 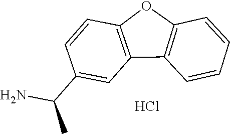 | 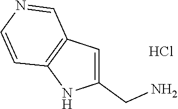 [SMILES: NCc1cc2cnccc2[nH]1 ; Cl] | ¹H NMR (400 MHz, CD₃OD) δ 8.69 (s, 1H), 8.10 (s, 2H), 8.02 (d, J = 6.2 Hz, 1H), 7.55 (s, 3H), 7.49-7.40 (m, 2H), 7.35 (d, J = 5.9 Hz, 2H), 7.27 (s, 1H), 7.15 (s, 1H), 7.04 (s, 1H), 6.71 (s, 1H), 6.39 (s, 1H), 5.34 (d, J = 6.7 Hz, 1H), 4.82 (s, 1H), 4.43 (s, 2H), 4.21 (s, 1H), 1.70 (d, J = 6.7 Hz, 3H). LC/MS (ESI) m/z: 587 (M + H)+. RT (Method A): 1.84 min. |

### Scheme 91. Synthesis of (S)—N-((1H-pyrrolo[3,2-c]pyridine-2-yl)methyl)-2-(5-((1-(dibenzo[b,d]furan-2-yl)ethyl)amino)-6-oxo-2-(pyridine-2-yl)pyrimidin-1(6H)-yl) acetamide (Compound 285)

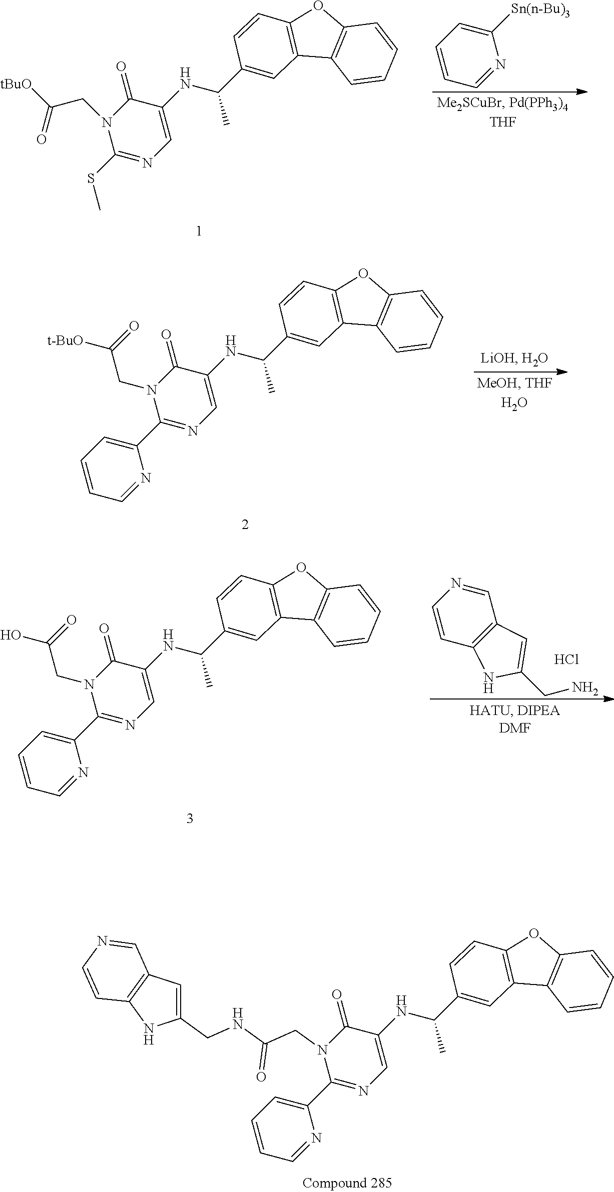

### Step 1: Tert-butyl (S)-2-(5-((1-(dibenzo[b,d]furan-2-yl)ethyl)amino)-6-oxo-2-(pyridine-2-yl)pyrimidin-1(6H)-yl)acetate (2)

To a mixture of tert-butyl (S)-2-(5-((1-(dibenzo[b,d]furan-2-yl)ethyl)amino)-2-(methylthio)-6-oxopyrimidin-1(6H)-yl)acetate (400 mg, 0.86 mmol) and 2-(tributylstannyl)pyridine (380 mg, 1.03 mmol) in THF (5 mL) was added Me₂S·CuBr (177 mg, 0.86 mmol), Pd(PPh₃)₄ (119 mg, 1.03 mmol) under N₂ atmosphere and the reaction mixture was stirred at 80° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-25% EtOAc in PE) to give the title compound (130 mg, yield 30.5%) as a yellow solid. LC/MS (ESI) m/z: 497 (M+H)⁺.

### Step 3: (S)-2-(5-((1-(dibenzo[b,d]furan-2-yl)ethyl)amino)-6-oxo-2-(pyridine-2-yl) pyrimidin-1(6H)-yl)acetic acid (3)

To a solution of tert-butyl (S)-2-(5-((1-(dibenzo[b,d]furan-2-yl)ethyl)amino)-6-oxo-2-(pyridine-2-yl)pyrimidin-1(6H)-yl)acetate (130 mg, 0.26 mmol) in MeOH (2 mL), THF (2 mL) and water (2 mL) was added LiOH (19 mg, 0.78 mmol) and the mixture was stirred at 60° C. for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (110 mg, yield 95.6%) as a yellow solid, which was used directly in the next step without further purification. LC/MS (ESI) m/z: 441 (M+H)⁺.

### Step 4: (S)—N-((1H-pyrrolo[3,2-c]pyridine-2-yl)methyl)-2-(5-((1-(dibenzo[b,d]furan-2-yl)ethyl)amino)-6-oxo-2-(pyridine-2-yl)pyrimidin-1(6H)-yl)acetamide (Compound 285)

To a mixture of (S)-2-(5-((1-(dibenzo[b,d]furan-2-yl)ethyl)amino)-6-oxo-2-(pyridine-2-yl)pyrimidin-1(6H)-yl)acetic acid (110 mg, 0.25 mmol) and (1H-pyrrolo[3,2-c]pyridine-2-yl)methanamine hydrochloride (55 mg, 0.30 mmol) in DMF (3 mL) was added DIPEA (194 mg, 1.50 mmol) and HATU (114 mg, 0.30 mmol) at 0° C. and the mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 285 (30 mg, yield 21.1%) as a white solid. ¹H NMR (400 MHz, DMSO-d₆) δ 11.23 (s, 1H), 8.72 (s, 1H), 8.58 (t, J=5.5 Hz, 1H), 8.46 (d, J=4.5 Hz, 1H), 8.23 (s, 1H), 8.10 (d, J=5.9 Hz, 2H), 7.80 (t, J=7.7 Hz, 1H), 7.72-7.61 (m, 4H), 7.51 (t, J=7.7 Hz, 1H), 7.41-7.35 (m, 2H), 7.28 (d, J=5.7 Hz, 1H), 7.03 (s, 1H), 6.20-6.10 (m, 2H), 5.21-5.11 (m, 2H), 4.75-4.68 (m, 1H), 4.32 (d, J=5.5 Hz, 2H), 1.62 (d, J=6.5 Hz, 3H). LC/MS (ESI) (m/z): 570 (M+H)⁺. RT (Method A): 1.60 min.

The following compounds were prepared based on Scheme 91:

| # | Reactant A | Reactant B | Reactant C |
|---|---|---|---|
| 286 | 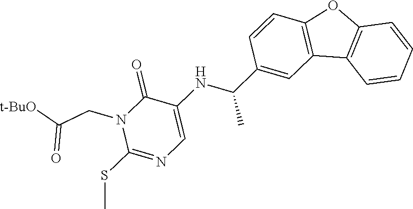 [SMILES: CSc1ncc(N[C@@H](C)c2ccc3oc4ccccc4c3c2)c(=O)n1CC(=O)OC(C)(C)C] | 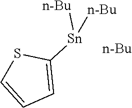 | 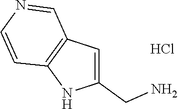 [SMILES: NCc1cc2cnccc2[nH]1 ; Cl] |
| 289^{a} | 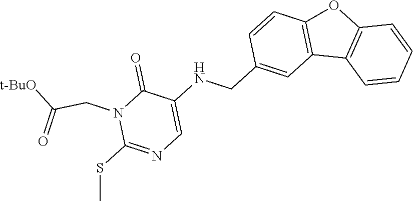 [SMILES: CSc1ncc(NCc2ccc3oc4ccccc4c3c2)c(=O)n1CC(=O)OC(C)(C)C] | 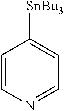 | 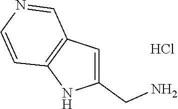 [SMILES: NCc1cc2cnccc2[nH]1 ; Cl] |
| 315^{a} | 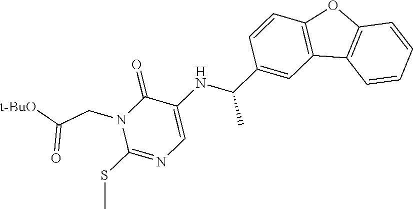 [SMILES: CSc1ncc(N[C@@H](C)c2ccc3oc4ccccc4c3c2)c(=O)n1CC(=O)OC(C)(C)C] | 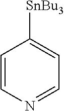 | 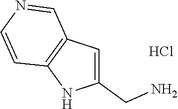 [SMILES: NCc1cc2cnccc2[nH]1 ; Cl] |
| 320^{b} | 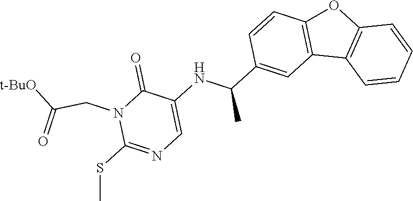 [SMILES: CSc1ncc(N[C@H](C)c2ccc3oc4ccccc4c3c2)c(=O)n1CC(=O)OC(C)(C)C] | 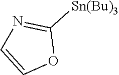 | 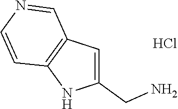 [SMILES: NCc1cc2cnccc2[nH]1 ; Cl] |
| 323 | 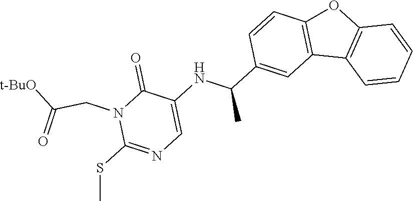 [SMILES: CSc1ncc(N[C@H](C)c2ccc3oc4ccccc4c3c2)c(=O)n1CC(=O)OC(C)(C)C] | 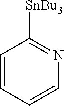 | 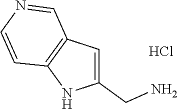 [SMILES: NCc1cc2cnccc2[nH]1 ; Cl] |
| 327 | 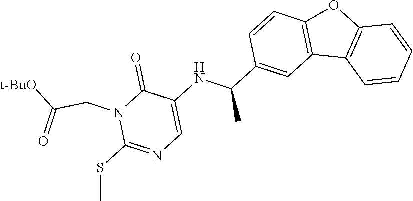 [SMILES: CSc1ncc(N[C@H](C)c2ccc3oc4ccccc4c3c2)c(=O)n1CC(=O)OC(C)(C)C] | 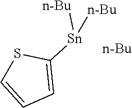 | 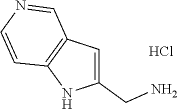 [SMILES: NCc1cc2cnccc2[nH]1 ; Cl] |
| 334 | 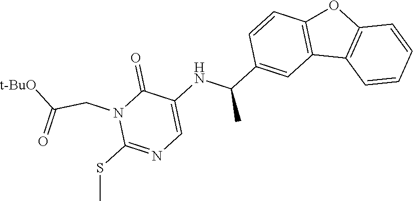 [SMILES: CSc1ncc(N[C@H](C)c2ccc3oc4ccccc4c3c2)c(=O)n1CC(=O)OC(C)(C)C] | 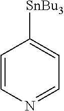 | 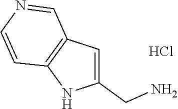 [SMILES: NCc1cc2cnccc2[nH]1 ; Cl] |
| 339^{c} | 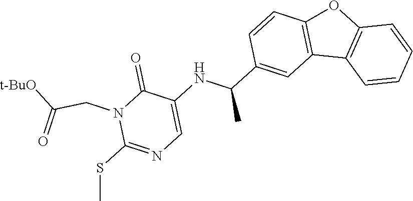 [SMILES: CSc1ncc(N[C@H](C)c2ccc3oc4ccccc4c3c2)c(=O)n1CC(=O)OC(C)(C)C] | 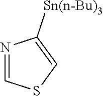 | 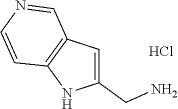 [SMILES: NCc1cc2cnccc2[nH]1 ; Cl] |
| 340^{d} | 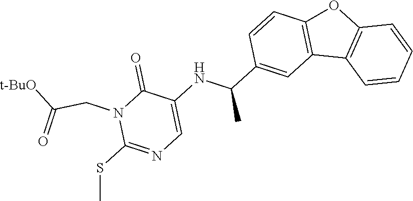 [SMILES: CSc1ncc(N[C@H](C)c2ccc3oc4ccccc4c3c2)c(=O)n1CC(=O)OC(C)(C)C] | 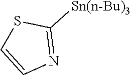 | 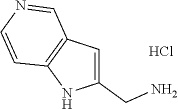 [SMILES: NCc1cc2cnccc2[nH]1 ; Cl] |
| 358^{d} | 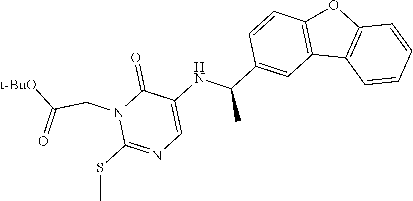 [SMILES: CSc1ncc(N[C@H](C)c2ccc3oc4ccccc4c3c2)c(=O)n1CC(=O)OC(C)(C)C] | 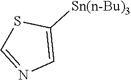 | 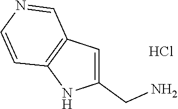 [SMILES: NCc1cc2cnccc2[nH]1 ; Cl] |
| 429^{e} | 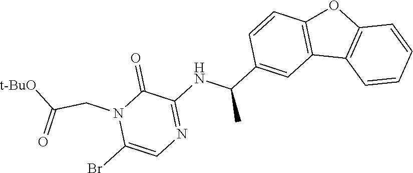 [SMILES: C[C@@H](Nc1ncc(Br)n(CC(=O)OC(C)(C)C)c1=O)c1ccc2oc3ccccc3c2c1] | 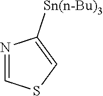 | 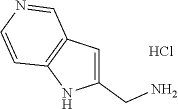 [SMILES: NCc1cc2cnccc2[nH]1 ; Cl] |
| 430^{e} | 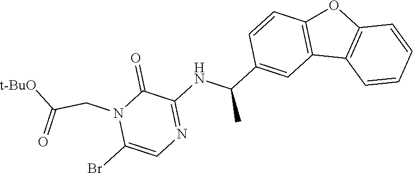 [SMILES: C[C@@H](Nc1ncc(Br)n(CC(=O)OC(C)(C)C)c1=O)c1ccc2oc3ccccc3c2c1] | 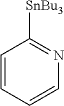 | 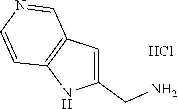 [SMILES: NCc1cc2cnccc2[nH]1 ; Cl] |
| 431^{e} | 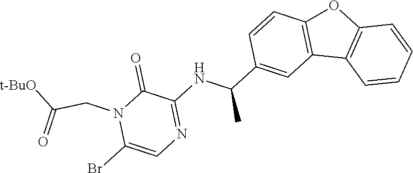 [SMILES: C[C@@H](Nc1ncc(Br)n(CC(=O)OC(C)(C)C)c1=O)c1ccc2oc3ccccc3c2c1] | 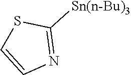 | 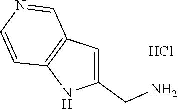 [SMILES: NCc1cc2cnccc2[nH]1 ; Cl] |
| 432^{e} | 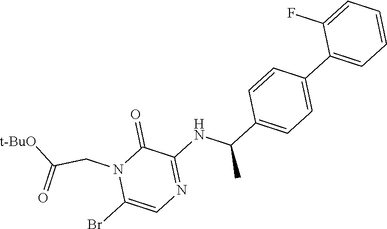 [SMILES: C[C@@H](Nc1ncc(Br)n(CC(=O)OC(C)(C)C)c1=O)c1ccc(-c2ccccc2F)cc1] | 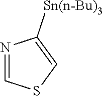 | 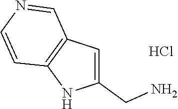 [SMILES: NCc1cc2cnccc2[nH]1 ; Cl] |
| 446^{e} | 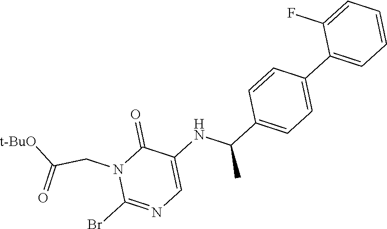 [SMILES: C[C@@H](Nc1cnc(Br)n(CC(=O)OC(C)(C)C)c1=O)c1ccc(-c2ccccc2F)cc1] | 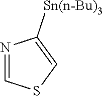 | 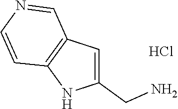 [SMILES: NCc1cc2cnccc2[nH]1 ; Cl] |
| 447 | 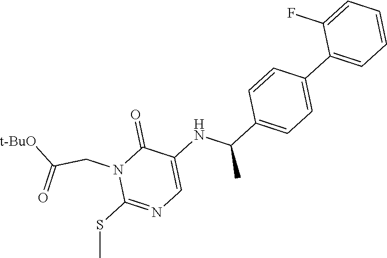 [SMILES: CSc1ncc(N[C@H](C)c2ccc(-c3ccccc3F)cc2)c(=O)n1CC(=O)OC(C)(C)C] | 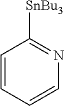 | 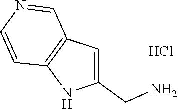 [SMILES: NCc1cc2cnccc2[nH]1 ; Cl] |
| 449^{e} | 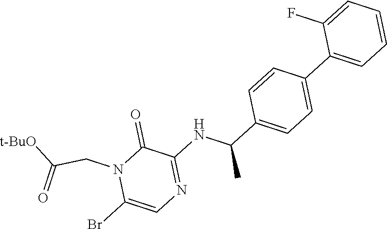 [SMILES: C[C@@H](Nc1ncc(Br)n(CC(=O)OC(C)(C)C)c1=O)c1ccc(-c2ccccc2F)cc1] | 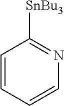 | 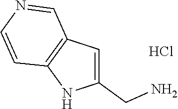 [SMILES: NCc1cc2cnccc2[nH]1 ; Cl] |

| # | Characterization Data |
|---|---|
| 286 | ¹H NMR (400 MHz, CD₃OD) δ 8.95 (s, 1H), |
| | 8.25 (d, J = 5.9 Hz, 1H), 8.09-7.98 (m, 2H), |
| | 7.74 (s, 1H), 7.65-7.51 (m, 4H), 7.48 (t, J = |
| | 7.8 Hz, 1H), 7.35 (t, J = 7.3 Hz, 1H), 7.25 (d, |
| | J = 3.3 Hz, 1H), 7.00 (t, J = 3.8 Hz, 1H), 6.96- |
| | 6.90 (m, 1H), 6.81 (s, 1H), 4.86-4.84 (m, |
| | 2H), 4.75-4.69 (m, 1H), 4.67 (s, 2H), 1.68 (d, |
| | J = 6.6 Hz, 3H). LC/MS (ESI) m/z: 575 |
| | (M + H)⁺. RT (Method A): 1.72 min. |
| 289^{a} | ¹H NMR (400 MHz, DMSO-d₆) δ 11.33 (s, 1H), |
| | 8.75 (dd, J = 8.8, 3.0 Hz, 2H), 8.56 (dd, J = |
| | 4.5, 1.5 Hz, 2H), 8.18-8.10 (m, 3H), 7.72- |
| | 7.66 (m, 2H), 7.59-7.47 (m, 2H), 7.44-7.38 |
| | (m, 3H), 7.33 (d, J = 5.6 Hz, 1H), 7.12 (d, J = |
| | 14.6 Hz, 1H), 6.62-6.51 (m, 1H), 6.32 (s, |
| | 1H), 4.56-4.50 (m, 4H), 4.42 (d, J = 5.0 Hz, |
| | 2H). LC/MS (ESI) m/z: 556 (M + H)⁺. RT |
| | (Method A): 1.41 min. |
| 315^{a} | ¹H NMR (400 MHz, DMSO-d₆) δ 11.33 (s, 1H), |
| | 8.75 (s, 2H), 8.54 (d, J = 4.7 Hz, 2H), 8.23 (s, |
| | 1H), 8.13 (d, J = 4.8 Hz, 1H), 8.10 (d, J = 7.6 |
| | Hz, 1H), 7.68 (s, 1H), 7.65 (d, J = 8.6 Hz, 2H), |
| | 7.51 (s, 1H), 7.39 (s, 3H), 7.34 (s, 1H), 7.02 |
| | (s, 1H), 6.32 (s, 1H), 6.11 (d, J = 7.0 Hz, 1H), |
| | 4.68 (d, J = 5.9 Hz, 1H), 4.53 (d, J = 29.1 Hz, |
| | 2H), 4.41 (s, 2H), 1.61 (d, J = 6.4 Hz, 3H). |
| | LC/MS (ESI) (m/z): 570 (M + H)⁺. RT (Method |
| | A): 1.50 min. |
| 320^{b} | ¹H NMR (400 MHz, CD₃OD) δ 8.70 (s, 1H), |
| | 8.11-8.05 (m, 2H), 7.99 (d, J = 7.8 Hz, 1H), |
| | 7.91 (s, 1H), 7.55 (d, J = 9.6 Hz, 3H), 7.46 (t, |
| | J = 7.7 Hz, 1H), 7.34 (m, 2H), 7.19 (s, 1H), |
| | 7.00 (s, 1H), 6.53 (s, 1H), 5.56 (s, 2H), 4.74 |
| | (q, J = 7.2 Hz, 1H), 4.56 (s, 2H), 1.68 (d, J = |
| | 6.6 Hz, 3H). LC/MS (ESI) m/z: 560 (M + H)⁺. |
| | RT (Method A): 1.55 min. |
| 323 | ¹H NMR (400 MHz, DMSO-d₆) δ 11.26 (s, 1H), |
| | 8.73 (s, 1H), 8.59 (s, 1H), 8.46 (s, 1H), 8.23 |
| | (s, 1H), 8.10 (d, J = 7.0 Hz, 2H), 7.80 (t, J = |
| | 7.4 Hz, 1H), 7.73-7.56 (m, 5H), 7.51 (t, J = |
| | 7.7 Hz, 1H), 7.41-7.36 (m, 2H), 7.30 (s, 1H), |
| | 7.04 (s, 1H), 6.20 (s, 1H), 5.16 (s, 2H), 4.71 |
| | (s, 1H), 4.32 (s, 2H), 1.62 (d, J = 6.3 Hz, 3H). |
| | LC/MS (ESI) (m/z): 570 (M + H)⁺. RT (Method |
| | A): 1.60 min. |
| 327 | ¹H NMR (400 MHz, CD₃OD) δ 8.72 (s, 1H), |
| | 8.11 (d, J = 5.1 Hz, 1H), 8.07 (s, 1H), 7.99 (d, |
| | J = 6.5 Hz, 1H), 7.50 (M, 5H), 7.40 (d, J = 4.6 |
| | Hz, 1H), 7.34 (t, J = 7.4 Hz, 1H), 7.21 (s, 1H), |
| | 6.95 (s, 1H), 6.92 (s, 1H), 6.54 (s, 1H), 4.82 |
| | (s, 2H), 4.69 (d, J = 6.2 Hz, 1H), 4.60 (s, 2H), |
| | 1.67 (d, J = 5.9 Hz, 3H). LC/MS (ESI) m/z: |
| | 575 (M + H)⁺. RT (Method A): 1.71 min. |
| 334 | ¹H NMR (400 MHz, DMSO-d₆) δ 11.33 (s, 1H), |
| | 8.75 (s, 2H), 8.54 (d, J = 3.9 Hz, 2H), 8.23 (s, |
| | 1H), 8.16-8.06 (m, 2H), 7.73-7.59 (m, 3H), |
| | 7.52 (t, J = 7.7 Hz, 1H), 7.40 (s, 3H), 7.33 (d, |
| | J= 5.5 Hz, 1H), 7.02 (s, 1H), 6.32 (s, 1H), 6.12 |
| | (d, J = 6.9 Hz, 1H), 4.74-4.65 (m, 1H), 4.58 |
| | (d, J = 16.6 Hz, 1H), 4.44 (m, 3H), 1.61 (d, J = |
| | 6.4 Hz, 3H). LC/MS (ESI) m/z: 570 (M + H)⁺. |
| | RT (Method A): 1.48 min. |
| 339^{c} | ¹H NMR (400 MHz, CD₃OD) δ 8.89 (s, 1H), |
| | 8.71 (s, 1H), 8.11-8.06 (m, 2H), 7.99 (d, J = |
| | 7.5 Hz, 1H), 7.89 (s, 1H), 7.55 (d, J = 9.4 Hz, |
| | 3H), 7.46 (t, J = 7.5 Hz, 1H), 7.38-7.32 (m, |
| | 2H), 6.94 (s, 1H), 6.48 (s, 1H), 5.10 (s, 2H), |
| | 4.70 (m, 1H), 4.53 (s, 2H), 1.68 (d, J = 6.4 Hz, |
| | 3H). LC/MS (ESI) (m/z): 576 (M + H)⁺. RT |
| | (Method A): 1.55 min. |
| 340^{d} | ¹H NMR (400 MHz, DMSO-d₆) δ 11.29 (s, 1H), |
| | 8.75 (d, J = 9.1 Hz, 2H), 8.23 (s, 1H), 8.10 (d, |
| | J = 5.4 Hz, 2H), 7.83 (s, 1H), 7.78 (s, 1H), 7.70 |
| | - 7.62 (m, 3H), 7.51 (t, J = 7.7 Hz, 1H), 7.40 |
| | (t, J = 7.2 Hz, 1H), 7.29 (d, J = 4.1 Hz, 1H), |
| | 7.07 (s, 1H), 6.43 (d, J = 6.6 Hz, 1H), 6.35 (s, |
| | 1H), 5.66-5.54 (m, 2H), 4.76-4.69 (m, 1H), |
| | 4.41 (s, 2H), 1.62 (d, J = 5.8 Hz, 3H). LC/MS |
| | (ESI) m/z: 576 (M + H)⁺. RT (Method A): 1.78 |
| | min. |
| 358^{d} | ¹H NMR (400 MHz, DMSO-d₆) δ 11.37 (s, 1H), |
| | 9.11 (s, 1H), 8.87 (d, J = 5.1 Hz, 1H), 8.74 (s, |
| | 1H), 8.21 (s, 1H), 8.13-8.08 (m, 2H), 7.99 (s, |
| | 1H), 7.69-7.65 (m, 2H), 7.61 (d, J = 7.7 Hz, |
| | 1H), 7.51 (t, J = 7.6 Hz, 1H), 7.39 (t, J = 7.2 |
| | Hz, 1H), 7.32 (d, J = 2.4 Hz, 1H), 7.01 (s, 1H), |
| | 6.36 (s, 1H), 6.16 (d, J = 7.1 Hz, 1H), 4.88- |
| | 4.73 (m, 2H), 4.68 (m, 1H), 4.46 (s, 2H), 1.61 |
| | (d, J = 5.8 Hz, 3H). LC/MS (ESI) (m/z): 576 |
| | (M + H)⁺. RT (Method A): 1.55 min. |
| 429^{e} | ¹H NMR (400 MHz, DMSO-d₆) δ 11.23 (s, 1H), |
| | 9.19 (d, J = 2.0 Hz, 1H), 8.73 (s, 1H), 8.57 (t, |
| | J = 5.7 Hz, 1H), 8.19 (s, 1H), 8.15-8.07 (m, |
| | 2H), 7.82-7.75 (m, 2H), 7.68 (d, J = 8.2 Hz, |
| | 1H), 7.64-7.61 (m, 2H), 7.54-7.47 (m, 1H), |
| | 7.39 (t, J = 7.5 Hz, 1H), 7.30 (d, J = 5.6 Hz, |
| | 1H), 6.92 (s, 1H), 6.25 (s, 1H), 5.44-5.22 (m, |
| | 1H), 4.89-4.65 (m, 2H), 4.34 (d, J = 5.6 Hz, |
| | 2H), 1.63 (d, J = 7.0 Hz, 3H). LC/MS (ESI) |
| | m/z: 576 (M + H)⁺. RT (Method A): 1.56 min. |
| 430^{e} | ¹H NMR (400 MHz, DMSO-d₆) δ 11.22 (s, 1H), |
| | 8.71 (s, 1H), 8.54 (t, J = 5.1 Hz, 2H), 8.20 (s, |
| | 1H), 8.14-8.07 (m, 2H), 7.85 (d, J = 8.3 Hz, |
| | 1H), 7.80-7.75 (m, 1H), 7.68 (d, J = 8.2 Hz, |
| | 1H), 7.64 (s, 2H), 7.51 (t, J = 7.6 Hz, 2H), 7.39 |
| | (t, J = 7.5 Hz, 1H), 7.36-7.31 (m, 1H), 7.28 |
| | (d, J = 5.7 Hz, 1H), 6.92 (s, 1H), 6.16 (s, 1H), |
| | 5.40-5.32 (m, 1H), 5.02-4.90 (m, 2H), 4.31 |
| | (d, J = 5.5 Hz, 2H), 1.63 (d, J = 6.9 Hz, 3H). |
| | LC/MS (ESI) m/z: 570 (M + H)⁺. RT (Method |
| | A): 1.58 min. |
| 431^{e} | ¹H NMR (400 MHz, CD₃OD) δ 8.68 (s, 1H), |
| | 8.12-8.06 (m, 2H), 8.01 (d, J = 7.5 Hz, 1H), |
| | 7.75 (d, J = 3.3 Hz, 1H), 7.60-7.53 (m, 3H), |
| | 7.47 (d, J = 7.1 Hz, 1H), 7.44 (d, J = 3.4 Hz, |
| | 1H), 7.38-7.31 (m, 2H), 7.23 (s, 1H), 6.43 (s, |
| | 1H), 5.44-5.35 (m, 1H), 5.21 (s, 2H), 4.50 (s, |
| | 2H), 1.71 (d, J = 6.9 Hz, 3H). LC/MS (ESI) |
| | (m/z): 576 (M + H)⁺. RT (Method A): 1.66 min. |
| 432^{e} | ¹H NMR (400 MHz, DMSO-d₆) δ 11.24 (s, 1H), |
| | 9.20 (d, J = 1.9 Hz, 1H), 8.73 (s, 1H), 8.57 (t, |
| | J = 5.6 Hz, 1H), 8.10 (d, J = 5.7 Hz, 1H), 7.82 |
| | (d, J = 1.9 Hz, 1H), 7.78 (d, J = 8.4 Hz, 1H), |
| | 7.54-7.47 (m, 5H), 7.42-7.37 (m, 1H), 7.32- |
| | 7.26 (m, 3H), 6.92 (s, 1H), 6.25 (s, 1H), 5.26- |
| | 5.17 (m, 1H), 4.87-4.75 (m, 2H), 4.35 (d, J = |
| | 5.5 Hz, 2H), 1.57 (d, J = 7.0 Hz, 3H). LC/MS |
| | (ESI) m/z: 580 (M + H)⁺. RT (Method A): 1.60 |
| | min. |
| 446^{e} | ¹H NMR (400 MHz, DMSO-d₆) δ 11.26 (s, 1H), |
| | 9.20 (d, J = 2.0 Hz, 1H), 8.73 (s, 1H), 8.58 (t, |
| | J = 5.6 Hz, 1H), 8.10 (d, J = 5.7 Hz, 1H), 7.82 |
| | (d, J = 2.0 Hz, 1H), 7.78 (d, J = 8.3 Hz, 1H), |
| | 7.54-7.51 (m, 3H), 7.50-7.47 (m, 2H), 7.42- |
| | 7.38 (m, 1H), 7.31-7.28 (m, 3H), 6.92 (s, |
| | 1H), 6.25 (s, 1H), 5.23-5.19 (m, 1H), 4.81 (d, |
| | J = 6.9 Hz, 2H), 4.35 (d, J = 5.6 Hz, 2H), 1.57 |
| | (d, J = 7.0 Hz, 3H). LC/MS (ESI) (m/z): 580 |
| | (M + H)⁺. RT (Method A): 1.63 min. |
| 447 | ¹H NMR (400 MHz, CD₃OD) d 8.70 (s, 1H), |
| | 8.39 (d, J = 4.8 Hz, 1H), 8.10 (d, J = 5.9 Hz, |
| | 1H), 7.74 (m, 2H), 7.53-7.43 (m, 5H), 7.37- |
| | 7.27 (m, 3H), 7.25-7.13 (m, 2H), 6.95 (s, 1H), |
| | 6.44 (s, 1H), 5.10 (d, J = 1.8 Hz, 2H), 4.59 (m, |
| | 1H), 4.50 (s, 2H), 1.63 (d, J = 6.8 Hz, 3H). |
| | LC/MS (ESI) m/z: 574 (M + H)⁺. RT (Method |
| | A): 1.64 min. |
| 449^{e} | ¹H NMR (400 MHz, DMSO-d₆) δ 11.24 (s, 1H), |
| | 8.72 (s, 1H), 8.56-8.53 (m, 2H), 8.15 (s, 1H), |
| | 8.10 (d, J = 5.7 Hz, 1H), 7.83 (d, J = 8.4 Hz, |
| | 1H), 7.81-7.76 (m, 1H), 7.54-7.53 (m, 1H), |
| | 7.53-7.51 (m, 2H), 7.50-7.49 (m, 2H), 7.41- |
| | 7.38 (m, 1H), 7.35-7.32 (m, 1H), 7.29 (d, J = |
| | 6.1 Hz, 2H), 7.27 (t, J = 1.4 Hz, 1H), 6.93 (s, |
| | 1H), 6.17 (s, 1H), 5.26-5.20 (m, 1H), 4.96 |
| | (m, 2H), 4.32 (m, 2H), 1.58 (d, J = 7.0 Hz, 3H). |
| | LC/MS (ESI) m/z: 574 (M + H)⁺. RT (Method |
| | A): 1.66 min. |

^{a}Copper disalicylate was used in place of copper bromide dimethyl sulfide in Step 1.
^{b}CuTC was used in place of copper bromide dimethyl sulfide in Step 1.
^{c}Step 1 was performed in the presence of copper(I) 3-methylsalicylate, K₂CO₃, LiCl, and Pd(PPh₃)₄ in THF.
^{d}Step 1 was performed in the presence of copper(I) 3-methylsalicylate, K₂CO₃, Cul, and Pd(PPh₃)₄ in THF.
^{e}Step 1 was performed in the presence of Pd(PPh₃)₄ in DMF.

### Scheme 92. Synthesis of N-((1H-pyrrolo[3,2-c]pyridine-2-yl)methyl)-2-(5-((dibenzo[b,d]furan-2-ylmethyl)amino)-2-(4-(2-(4-fluorophenoxy)ethoxy)phenyl)-6-oxopyrimidin-1(6H)-yl)acetamide (Compound 154)

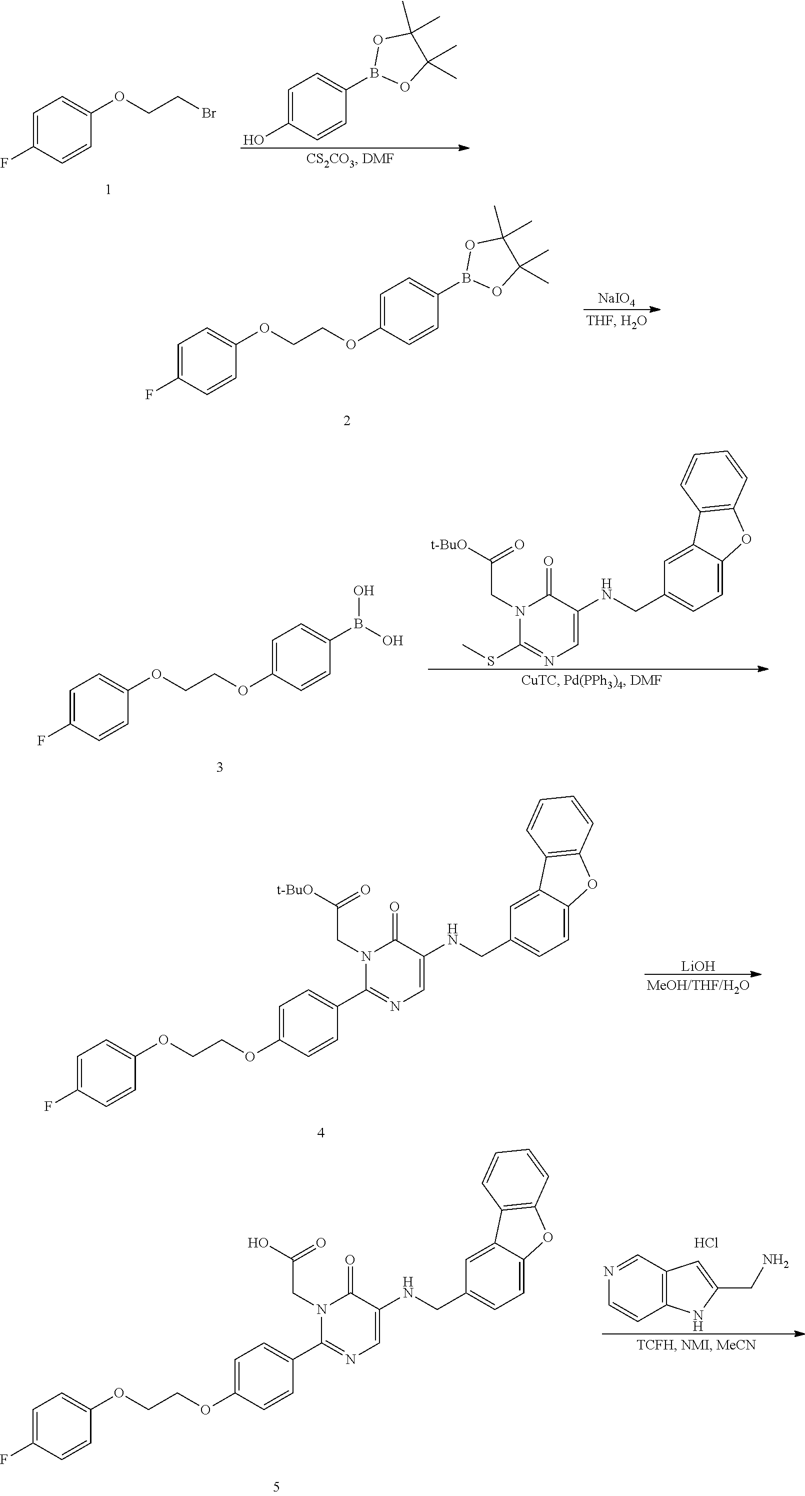

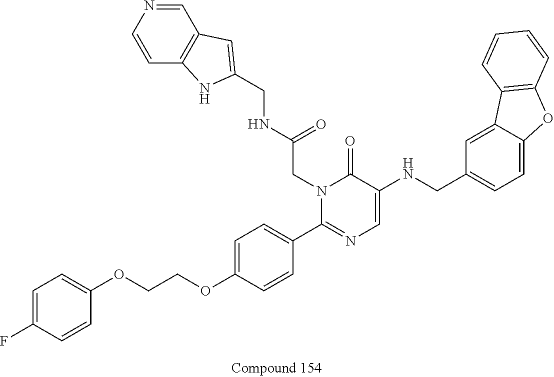 [SMILES: O=C(Cn1c(-c2ccc(OCCOc3ccc(F)cc3)cc2)ncc(NCc2ccc3oc4ccccc4c3c2)c1=O)NCc1cc2cnccc2[nH]1]

### Step 1: 2-(4-(2-(4-Fluorophenoxy)ethoxy)phenyl)-4,4,5,5-tetramethyl-1,3,2-dioxaborolane (2)

To a mixture of 1-(2-bromoethoxy)-4-fluorobenzene (440 mg, 2.0 mmol) and 4-(4,4,5,5-tetramethyl-1,3,2-dioxaborolan-2-yl)phenol (884 mg, 4.0 mmol) in DMF (4.4 mL) was added Cs₂CO₃ (1.955 g, 6.0 mmol) under N₂ atmosphere, the reaction mixture was stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-100% EtOAc in PE) to give title compound (440 mg, yield 61.4%) as a white solid. LC/MS (ESI) m/z: 359 (M+H)⁺.

### Step 2: (4-(2-(4-Fluorophenoxy)ethoxy)phenyl)boronic acid (3)

To a solution of 2-(4-(2-(4-fluorophenoxy)ethoxy)phenyl)-4,4,5,5-tetramethyl-1,3,2-dioxaborolane (400 mg, 1.12 mmol) in THF (2.0 mL) and water (2.0 mL) was added NaIO₄ (477 mg, 2.23 mmol) at room temperature and the mixture was stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-20% DCM in MeOH) to give title compound (205 mg, yield 66.3%) as a white solid. LC/MS (ESI) (m/z): 277 (M+H)⁺.

### Step 3: Tert-butyl 2-(5-((dibenzo[b,d]furan-2-ylmethyl)amino)-2-(4-(2-(4-fluorophenoxy)ethoxy)phenyl)-6-oxopyrimidin-1(6H)-yl)acetate (4)

To a mixture of tert-butyl 2-(5-((dibenzo[b,d]furan-2-ylmethyl)amino)-2-(methylthio)-6-oxopyrimidin-1(6H)-yl)acetate (100 mg, 0.22 mmol) and (4-(2-(4-fluorophenoxy) ethoxy)phenyl)boronic acid (122 mg, 0.44 mmol) in DMF (1.0 mL) was added CuTC (92 mg, 0.48 mmol) and Pd(PPh₃)₄ (35 mg, 0.03 mmol) under N₂ atmosphere, the reaction mixture was degassed under N₂ atmosphere and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-100% EtOAc in PE) to give title compound (37 mg, yield 26.5%) as a yellow solid. LC/MS (ESI) m/z: 636 (M+H)⁺.

### Step 4: 2-(5-((dibenzo[b,d]furan-2-ylmethyl)amino)-2-(4-(2-(4-fluorophenoxy) ethoxy)phenyl)-6-oxopyrimidin-1(6H)-yl)acetic acid (5)

To a mixture of tert-butyl 2-(5-((dibenzo[b,d]furan-2-ylmethyl)amino)-2-(4-(2-(4-fluorophenoxy)ethoxy)phenyl)-6-oxopyrimidin-1(6H)-yl)acetate (37 mg, 0.06 mmol) in MeOH (0.2 mL), THF (0.2 mL) and water (0.4 mL) was added LiOH·H₂O (9 mg, 0.21 mmol) and the reaction mixture was stirred at 50° C. overnight. The mixture was acidified with 1 N aq. HCl to pH˜3 and extracted with EtOAc twice. The combined organic layers were washed with brine, dried over anhydrous Na₂SO₄, filtered, and concentrated to give title compound (30 mg, yield 89.2%) as a white solid. LC/MS (ESI) (m/z): 580 (M+H)⁺.

### Step 5: N-((1H-pyrrolo[3,2-c]pyridine-2-yl)methyl)-2-(5-((dibenzo[b,d]furan-2-ylmethyl)amino)-2-(4-(2-(4-fluorophenoxy)ethoxy)phenyl)-6-oxopyrimidin-1(6H)-yl)acetamide (Compound 154)

To a mixture of 2-(5-((dibenzo[b,d]furan-2-ylmethyl)amino)-2-(4-(2-(4-fluorophenoxy) ethoxy)phenyl)-6-oxopyrimidin-1(6H)-yl)acetic acid (30 mg, 0.05 mmol) and (1H-pyrrolo[3,2-c]pyridine-2-yl)methanamine hydrochloride (26 mg, 0.14 mmol) in MeCN (1.0 mL) was added NMI (7 mg, 0.08 mmol) and TCFH (22 mg, 0.08 mmol) under N₂ atmosphere and the reaction mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=9:1) and further purified by prep-HPLC to give Compound 154 (2.2 mg, yield 6.0%) as a white solid. ¹H NMR (400 MHz, CD₃OD) δ 9.03 (d, J=6.2 Hz, 1H), 8.28 (t, J=6.7 Hz, 1H), 8.07 (s, 1H), 8.00 (d, J=7.5 Hz, 1H), 7.89 (d, J=6.3 Hz, 1H), 7.58 (m, 3H), 7.49-7.32 (m, 5H), 7.11 (m, 1H), 7.02-6.98 (m, 3H), 6.98-6.92 (m, 3H), 6.89 (s, 1H), 4.71 (s, 2H), 4.65 (s, 2H), 4.60 (s, 2H), 4.32 (s, 2H), 4.31 (s, 2H). LC/MS (ESI) m/z: 709 (M+H)⁺. RT (Method A): 2.04 min.

### Scheme 93. Synthesis of N-((1H-pyrrolo[3,2-c]pyridine-2-yl)methyl)-2-(5-((dibenzo[b,d]furan-2-ylmethyl)amino)-2-(4-(2-hydroxyacetamido)phenyl)-6-oxopyrimidin-1(6H)-yl) acetamide (Compound 147)

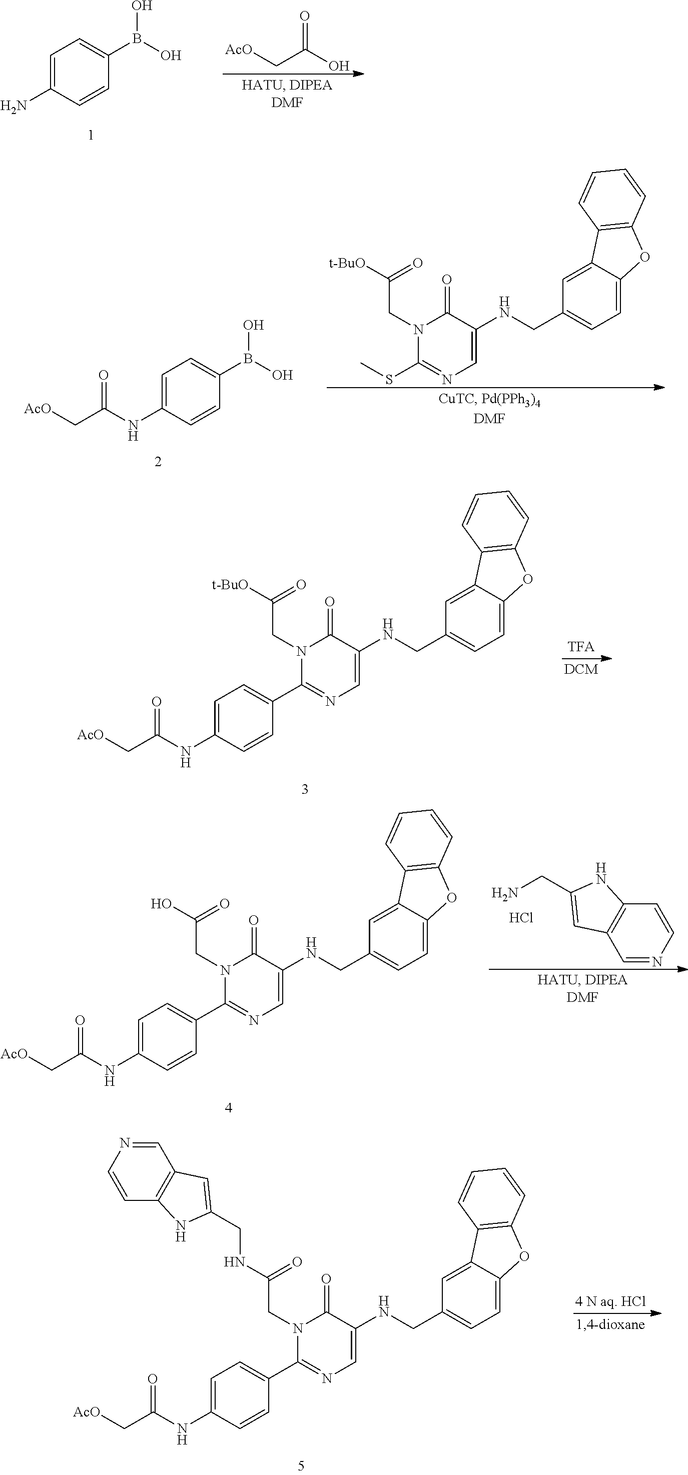

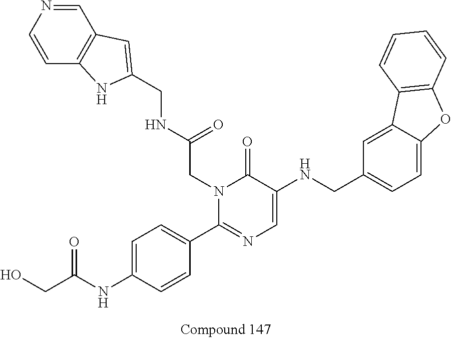 [SMILES: O=C(Cn1c(-c2ccc(NC(=O)CO)cc2)ncc(NCc2ccc3oc4ccccc4c3c2)c1=O)NCc1cc2cnccc2[nH]1]

### Step 1: (4-(2-Acetoxyacetamido)phenyl)boronic acid (2)

To a mixture of (4-Aminophenyl)boronic acid (550 mg, 4.01 mmol) and 2-acetoxyacetic acid (568 mg, 4.81 mmol) in DMF (1 mL) was added HATU (1.98 g, 5.21 mmol), DIPEA (2.07 g, 16.01 mmol) and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (850 mg, yield 98.4%) as a colorless oil. LC/MS (ESI) m/z: 238 (M+H)⁺.

### Step 2: Tert-butyl 2-(2-(4-(2-acetoxyacetamido)phenyl)-5-((dibenzo[b,d]furan-2-ylmethyl)amino)-6-oxopyrimidin-1(6H)-yl)acetate (3)

To a mixture of (4-(2-Acetoxyacetamido)phenyl)boronic acid (60 mg, 0.25 mmol) and tert-butyl 2-(5-((dibenzo[b,d]furan-2-ylmethyl)amino)-2-(methylthio)-6-oxopyrimidin-1(6H)-yl)acetate (96 mg, 0.21 mmol) in DMF (2 mL) was added CuTC (90 mg, 0.47 mmol), Pd(PPh₃)₄ (25 mg, 0.02 mmol). The reaction mixture was degassed under N₂ atmosphere for three times and stirred at 80° C. overnight. The mixture was diluted with water and extracted with EtOAc twice. The combined organic layers were washed with saturated aq. NaHCO₃ solution and brine, dried over anhydrous Na₂SO₄, filtered, and concentrated under reduced pressure to dryness. The residue was purified by flash chromatography (silica gel, 0-17% EtOAc in PE) to give the title compound (60 mg, yield 39.7%) as a yellow oil. LC/MS (ESI) m/z: 597 (M+H)⁺.

### Step 3: 2-(2-(4-(2-acetoxyacetamido)phenyl)-5-((dibenzo[b,d]furan-2-ylmethyl) amino)-6-oxopyrimidin-1(6H)-yl)acetic acid (4)

To a solution of tert-butyl 2-(2-(4-(2-acetoxyacetamido)phenyl)-5-((dibenzo[b,d]furan-2-ylmethyl)amino)-6-oxopyrimidin-1(6H)-yl)acetate (60 mg, 0.10 mmol) in DCM (3 mL) was added TFA (1 mL). The reaction stirred at room temperature for 1 hour. The mixture was concentrated under reduced pressure to dryness to give the title compound (50 mg, yield 92.1%) as a yellow oil. LC/MS (ESI) m/z: 541 (M+H)⁺.

### Step 4: 2-((4-(1-(2-(((1H-pyrrolo[3,2-c]pyridine-2-yl)methyl)amino)-2-oxoethyl)-5-((dibenzo[b,d]furan-2-ylmethyl)amino)-6-oxo-1,6-dihydropyrimidin-2-yl)phenyl) amino)-2-oxoethyl acetate (5)

To a mixture of 2-(2-(4-(2-acetoxyacetamido)phenyl)-5-((dibenzo[b,d]furan-2-ylmethyl)amino)-6-oxopyrimidin-1(6H)-yl)acetic acid (50 mg, 0.09 mmol) and (1H-pyrrolo[3,2-c]pyridine-2-yl)methanamine hydrochloride (20 mg, 0.11 mmol) in DMF (2 mL) was added HATU (60 mg, 0.16 mmol), DIPEA (70 mg, 0.54 mmol) and the mixture was stirred at room temperature for 0.5 hour. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (EtOAc:PE=1:1) to give the title compound (60 mg, yield 96.8%) as a yellow oil. LC/MS (ESI) m/z: 670 (M+H)⁺

### Step 5: N-((1H-pyrrolo[3,2-c]pyridine-2-yl)methyl)-2-(5-((dibenzo[b,d]furan-2-ylmethyl)amino)-2-(4-(2-hydroxyacetamido)phenyl)-6-oxopyrimidin-1(6H)-yl)acetamide (Compound 147)

To a solution of 2-((4-(1-(2-(((1H-pyrrolo[3,2-c]pyridine-2-yl)methyl)amino)-2-oxoethyl)-5-((dibenzo[b,d]furan-2-ylmethyl)amino)-6-oxo-1,6-dihydropyrimidin-2-yl) phenyl)amino)-2-oxoethyl acetate (60 mg, 0.09 mmol) in 1,4-dioxane (3 mL) was added conc. HCl (1 mL) and the reaction stirred at room temperature for 1 hour. The mixture was concentrated to dryness and the residue was purified by prep-HPLC to give Compound 147 (1.9 mg, yield 3.0%) as a white solid. ¹H NMR (400 MHz, CD₃OD) δ 8.75 (s, 1H), 8.13-8.10 (m, 1H), 8.07 (s, 1H), 8.01-7.99 (m, 1H), 7.67-7.62 (m, 3H), 7.58-7.54 (m, 3H), 7.50-7.46 (m, 1H), 7.43 (s, 2H), 7.41 (s, 1H), 7.36-7.33 (m, 1H), 7.11 (s, 1H), 5.35-5.33 (m, 1H), 4.68 (s, 2H), 4.59-4.56 (m, 5H). LC/MS (ESI) m/z: 628 (M+H)⁺. RT (Method A): 1.40 min.

### Scheme 94. Synthesis of (S)—N-((1H-pyrrolo[3,2-c]pyridine-2-yl)methyl)-2-(3-((1-(dibenzo[b,d]furan-2-yl)ethyl)amino)-2-oxopyrazin-1(2H)-yl)acetamide (Compound 299)

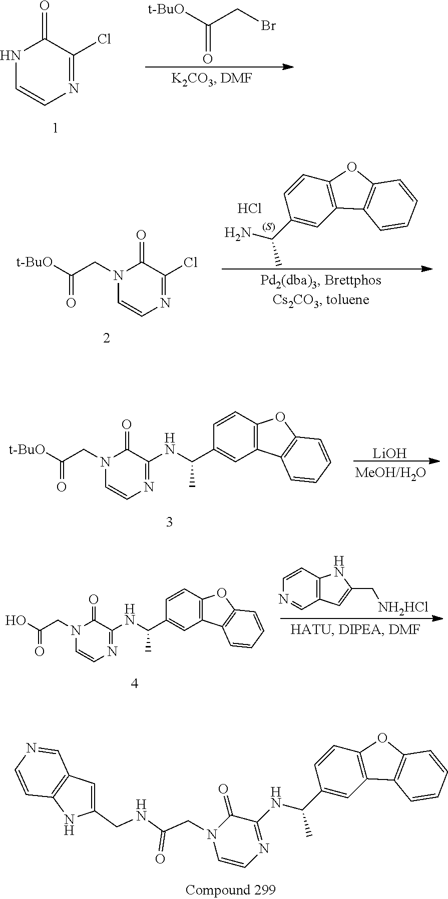

### Step 1: Tert-butyl 2-(3-chloro-2-oxopyrazin-1(2H)-yl)acetate (2)

To a mixture of 3-chloropyrazin-2(1H)-one (200 mg, 1.53 mmol) and tert-butyl 2-bromoacetate (448 mg, 2.29 mmol) in DMF (5 mL) was added K₂CO₃ (423 mg, 3.06 mmol) and the reaction mixture was stirred at room temperature overnight. The mixture was quenched with saturated aq.NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (250 mg, yield 66.6%) as a white solid. LC/MS (ESI) m/z: 245 (M+H)⁺.

### Step 2: Tert-butyl (S)-2-(3-((1-(dibenzo[b,d]furan-2-yl)ethyl)amino)-2-oxopyrazin-1(2H)-yl)acetate (3)

To a mixture of tert-butyl 2-(3-chloro-2-oxopyrazin-1(2H)-yl)acetate (100 mg, 0.40 mmol) and (S)-1-(dibenzo[b,d]furan-2-yl)ethan-1-amine hydrochloride (95 mg, 0.38 mmol) in toluene (5 mL) was added Cs₂CO₃ (400 mg, 1.22 mmol), Brettphos (22 mg, 0.04 mmol), Pd₂(dba)₃ (37 mg, 0.04 mmol) under N₂ atmosphere and the reaction mixture was stirred at 120° C. overnight. The mixture was diluted with EtOAc, washed with brine, dried over anhydrous Na₂SO₄, filtered, and concentrated under reduced pressure to dryness. The residue was purified by flash chromatography (silica gel, 0-35% EtOAc in PE) to give the title compound (30 mg, yield 17.5%) as a yellow solid. LC/MS (ESI) m/z: 420 (M+H)⁺.

### Step 3: (S)-2-(3-((1-(dibenzo[b,d]furan-2-yl)ethyl)amino)-2-oxopyrazin-1(2H)-yl)acetic acid (4)

To a solution of tert-butyl (S)-2-(3-((1-(dibenzo[b,d]furan-2-yl)ethyl)amino)-2-oxopyrazin-1(2H)-yl)acetate (30 mg, 0.071 mmol) in MeOH/water (2.0 mL, v/v=4/1) was added LiOH·H₂O (6 mg, 0.14 mmol) and the reaction mixture was stirred at 60° C. for 2 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (25 mg, yield 96.2%) as a yellow solid, which was used directly in the next step without further purification. LC/MS (ESI) m/z: 364 (M+H)⁺.

### Step 4: (S)—N-((1H-pyrrolo[3,2-c]pyridine-2-yl)methyl)-2-(3-((1-(dibenzo[b,d]furan-2-yl)ethyl)amino)-2-oxopyrazin-1(2H)-yl)acetamide (Compound 299)

To a mixture of (S)-2-(3-((1-(dibenzo[b,d]furan-2-yl)ethyl)amino)-2-oxopyrazin-1(2H)-yl)acetic acid (25 mg, 0.069 mmol) and (1H-pyrrolo[3,2-c]pyridine-2-yl)methanamine hydrochloride (15 mg, 0.081 mmol) in DMF (2 mL) was added DIPEA (44 mg, 0.34 mmol) and HATU (31 mg, 0.081 mmol) and the reaction mixture was stirred at room temperature for 1 hour. The mixture was concentrated under reduced pressure to dryness. The residue was purified by prep-HPLC to give Compound 299 (5.0 mg, yield 14.7%) as a white solid. ¹H NMR (400 MHz, DMSO-d₆) δ 11.43 (s, 1H), 8.76 (s, 2H), 8.13 (dd, J=16.2, 8.4 Hz, 3H), 7.67 (d, J=8.2 Hz, 1H), 7.61 (t, J=7.0 Hz, 2H), 7.51 (t, J=7.8 Hz, 1H), 7.46 (d, J=8.3 Hz, 1H), 7.41-7.34 (m, 2H), 6.75 (d, J=4.5 Hz, 1H), 6.70 (d, J=4.5 Hz, 1H), 6.46 (s, 1H), 5.25 (q, J=6.7 Hz, 1H), 4.60-4.51 (m, 2H), 4.47 (d, J=4.8 Hz, 2H), 1.59 (d, J=6.9 Hz, 3H). LC/MS (ESI) m/z: 493 (M+H)⁺. RT (Method A): 1.43 min. Compounds 329 and 346 were prepared based on 94:

| # | Reactant A | Reactant B | Reactant C | Reactant D | Characterization Data |
|---|---|---|---|---|---|
| 329 | 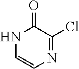 | 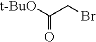 | 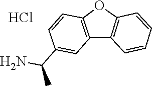 [SMILES: Cl ; C[C@@H](N)c1ccc2oc3ccccc3c2c1] | 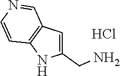 [SMILES: NCc1cc2cnccc2[nH]1 ; Cl] | ¹H NMR (400 MHz, DMSO-d₆) δ 11.34 (s, 1H), 8.74 (d, J = 6.6 Hz, 2H), 8.16 (s, 1H), 8.11 (d, J = 6.6 Hz, 2H), 7.67 (d, J = 8.6 Hz, 1H), 7.63-7.57 (m, 2H), 7.49 (dd, J = 19.5, 7.9 Hz, 2H), 7.39 (t, J = 7.4 Hz, 1H), 7.32 (s, 1H), 6.74 (s, 1H), 6.70 (s, 1H), 6.43 (s, 1H), 5.26 (d, |
| | | | | | J = 7.8 Hz, 1H), 4.53 (d, J = 16.9 |
| | | | | | Hz, 2H), 4.46 (s, 2H), 1.59 |
| | | | | | (d, J = 6.2 Hz, 3H). LC/MS (ESI) |
| | | | | | m/z: 493 (M + H)⁺. RT (Method |
| | | | | | A): 1.44 min. |
| 346^{a} | 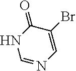 | 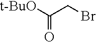 | 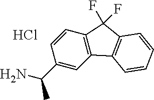 [SMILES: Cl ; C[C@@H](N)c1ccc2c(c1)-c1ccccc1C2(F)F] | 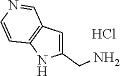 [SMILES: NCc1cc2cnccc2[nH]1 ; Cl] | ¹H NMR (400 MHz, DMSO-d₆) δ 11.37 (s, 1H), 8.85 (s, 1H), 8.75 (s, 1H), 8.15-8.09 (m, 1H), 7.91 (s, 1H), 7.78 (d, J = 7.4 Hz, 1H), 7.72 (s, 1H), 7.71-7.63 (m, 2H), 7.58 (t, J = 7.3 Hz, 1H), 7.48-7.40 (m, 2H), 7.33 (d, J = 4.9 Hz, 1H), 6.83 (s, 1H), 6.45 (s, |
| | | | | | 1H), 5.84 (d, J = 6.5 Hz, 1H), 4.71- |
| | | | | | 4.59 (m, 2H), 4.55-4.45 (m, 3H), |
| | | | | | 1.54 (d, J = 6.0 Hz, 3H). LC/MS |
| | | | | | (ESI) m/z: 527 (M + H)⁺. RT |
| | | | | | (Method A): 1.49 min. |

^{a}Step 1 was performed in the presence of CaH₂ in THF, and BINAP was used in place of BrettPhos in Step 2.

### Scheme 95. Synthesis of (S)—N-((1H-pyrrolo[3,2-c]pyridine-2-yl)methyl)-2-(5-((1-(dibenzo[b,d]furan-2-yl)ethyl)amino)-6-oxo-2-(3-(trifluoromethyl)-5,6-dihydro-[1,2,4]triazolo [4,3-a]pyrazin-7(8H)-yl)pyrimidin-1(6H)-yl)acetamide (Compound 300)

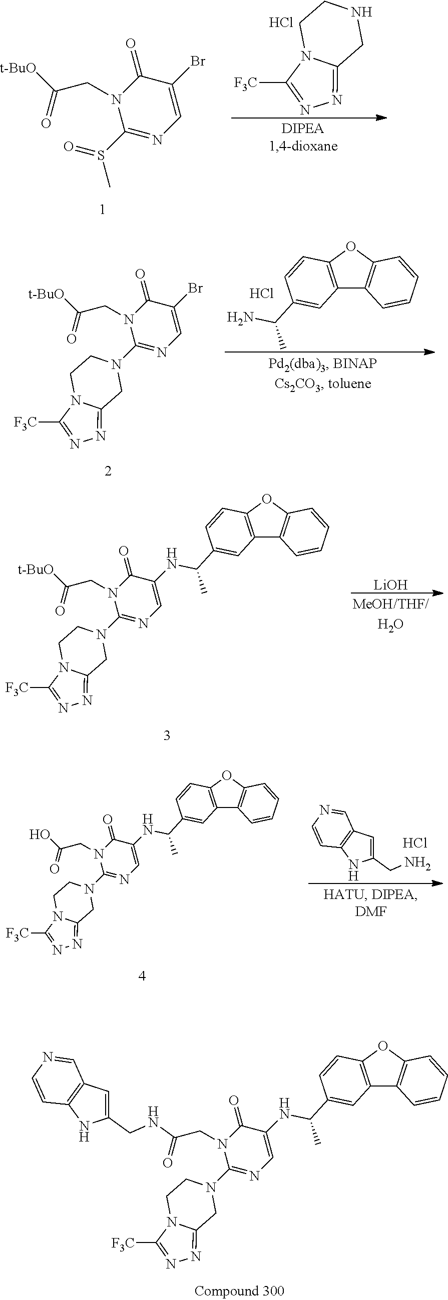

### Step 1: Tert-butyl 2-(5-bromo-6-oxo-2-(3-(trifluoromethyl)-5,6-dihydro-[1,2,4]triazolo[4,3-a]pyrazin-7(8H)-yl)pyrimidin-1(6H)-yl)acetate (2)

To a solution of tert-butyl 2-(5-bromo-2-(methylsulfinyl)-6-oxopyrimidin-1(6H)-yl)acetate (1 g, 2.85 mmol) in 1,4-dioxane (7 mL) was added 3-(trifluoromethyl)-5,6,7,8-tetrahydro-[1,2,4]triazolo[4,3-a]pyrazine hydrochloride (514 mg, 6.05 mmol) and DIPEA (1.75 mL, 10 mmol) and the reaction mixture was stirred at 110° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-80% EtOAc in PE) to give the title compound (179 mg, yield 13.1%) as a yellow solid. LC/MS (ESI) m/z: 479 (M+H)⁺.

### Step 2: Tert-butyl (S)-2-(5-((1-(dibenzo[b,d]furan-2-yl)ethyl)amino)-6-oxo-2-(3-(trifluoromethyl)-5,6-dihydro-[1,2,4]triazolo[4,3-a]pyrazin-7(8H)-yl)pyrimidin-1(6H)-yl)acetate (3)

To a mixture of tert-butyl 2-(5-bromo-6-oxo-2-(3-(trifluoromethyl)-5,6-dihydro-[1,2,4]triazolo[4,3-a]pyrazin-7(8H)-yl)pyrimidin-1(6H)-yl)acetate (150 mg, 0.31 mmol) and (S)-1-(dibenzo[b,d]furan-2-yl)ethan-1-amine hydrochloride (66 mg, 0.31 mmol) in toluene (3 mL) was added Cs₂CO₃ (302 mg, 0.93 mmol), BINAP (56 mg, 0.09 mmol) and Pd₂(dba)₃ (85 mg, 0.09 mmol) at 0° C. under N₂ atmosphere, the reaction mixture was degassed under N₂ atmosphere for three times and stirred at 110° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-80% EtOAc in PE) to give the title compound (63 mg, yield 32.9%) as a yellow solid. LC/MS (ESI) m/z: 610 (M+H)⁺.

### Step 3: (S)-2-(5-((1-(dibenzo[b,d]furan-2-yl)ethyl)amino)-6-oxo-2-(3-(trifluoromethyl)-5,6-dihydro-[1,2,4]triazolo[4,3-a]pyrazin-7(8H)-yl)pyrimidin-1(6H)-yl)acetic acid (4)

To a solution of tert-butyl (S)-2-(5-((1-(dibenzo[b,d]furan-2-yl)ethyl)amino)-6-oxo-2-(3-(trifluoromethyl)-5,6-dihydro-[1,2,4]triazolo[4,3-a]pyrazin-7(8H)-yl)pyrimidin-1(6H)-yl)acetate (63 mg, 0.1 mmol) in MeOH/THF/water (2 mL, 2/1/1) was added LiOH (7.4 mg, 0.3 mmol) and the reaction mixture was stirred at 50° C. for 12 hours. The mixture was acidified with 1N aq. HCl to pH˜3 and extracted with EtOAc twice. The combined organic layers were dried over anhydrous Na₂SO₄, filtered, and concentrated under reduced pressure to give the title compound (57 mg, yield 99.6%) as a yellow solid, which was used directly in the next step without further purification. LC/MS (ESI) m/z: 554 (M+H)⁺.

### Step 4: (S)—N-((1H-pyrrolo[3,2-c]pyridine-2-yl)methyl)-2-(5-((1-(dibenzo[b,d]furan-2-yl)ethyl)amino)-6-oxo-2-(3-(trifluoromethyl)-5,6-dihydro-[1,2,4]triazolo[4,3-a]pyrazin-7(8H)-yl)pyrimidin-1(6H)-yl)acetamide (Compound 300)

To a mixture of (S)-2-(5-((1-(dibenzo[b,d]furan-2-yl)ethyl)amino)-6-oxo-2-(3-(trifluoromethyl)-5,6-dihydro-[1,2,4]triazolo[4,3-a]pyrazin-7(8H)-yl)pyrimidin-1(6H)-yl)acetic acid (57 mg, 0.1 mmol) and (1H-pyrrolo[3,2-c]pyridine-2-yl)methanamine hydrochloride (27 mg, 0.15 mmol) in DMF (3 mL) was added DIPEA (71 mg, 0.55 mmol) and HATU (57 mg, 0.15 mmol) under N₂ atmosphere and the reaction mixture was stirred at room temperature for 1 hour. The mixture was concentrated to dryness and the residue was purified by prep-HPLC to give Compound 300 (5.0 mg, yield 7.1%) as a yellow solid. ¹H NMR (400 MHz, DMSO-d₆) δ 11.33-11.30 (m, 1H), 8.87-8.81 (m, 1H), 8.72 (s, 1H), 8.19 (s, 1H), 8.12 (d, J=5.6 Hz, 1H), 8.08 (d, J=7.4 Hz, 1H), 7.68 (d, J=8.0 Hz, 1H), 7.65-7.58 (m, 2H), 7.51 (t, J=7.7 Hz, 1H), 7.39 (t, J=7.6 Hz, 1H), 7.32 (d, J=5.5 Hz, 1H), 6.77 (s, 1H), 6.39 (s, 1H), 5.72-5.69 (m, 1H), 4.75 (s, 2H), 4.59-4.54 (m, 1H), 4.45 (s, 2H), 4.32 (s, 2H), 4.11 (dt, J=5.7, 4.1 Hz, 2H), 3.43-3.38 (m, 2H), 1.57 (d, J=6.5 Hz, 3H). LC/MS (ESI) m/z: 683 (M+H)⁺. RT (Method A): 1.67 min.

Compound 313 was prepared based on Scheme 95:

| # | Reactant A | Reactant B | Reactant C | Reactant D | Characterization Data |
|---|---|---|---|---|---|
| 313 | 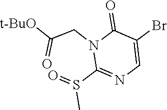 [SMILES: CS(=O)c1ncc(Br)c(=O)n1CC(=O)OC(C)(C)C] | 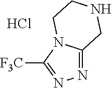 [SMILES: Cl ; FC(F)(F)c1nnc2n1CCNC2] | 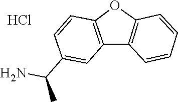 [SMILES: Cl ; C[C@@H](N)c1ccc2oc3ccccc3c2c1] | 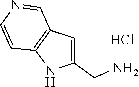 [SMILES: NCc1cc2cnccc2[nH]1 ; Cl] | ¹H NMR (400 MHz, CD₃OD) δ 8.77 (s, 1H), 8.16-8.13 (m, 1H), 8.03 (s, 1H), 7.97 (d, J = 7.8 Hz, 1H), 7.56-7.54 (m, 1H), 7.52-7.46 (m, 4H), 7.33 (t, J = 7.2 Hz, 1H), 6.76 (s, |
| | | | | | 1H), 6.62 (s, 1H), |
| | | | | | 4.89 (s, 2H), 4.62(s, |
| | | | | | 2H), 4.61-4.57 |
| | | | | | (m, 1H), 4.40 (s, |
| | | | | | 2H), 4.14-4.10 (m, |
| | | | | | 2H), 3.51-3.48 (m, |
| | | | | | 2H), 1.64 (d, J = |
| | | | | | 6.6 Hz, 3H). |
| | | | | | LC/MS (ESI) m/z: 683 |
| | | | | | (M + H)⁺. RT |
| | | | | | (Method A): 1.69 min. |

### Scheme 96. Synthesis of N-((1H-pyrrolo[3,2-c]pyridine-2-yl)methyl)-2-(3-cyano-4-((3-phenylpropyl)amino)-[1,1′-biphenyl]-2-yl)acetamide (Compound 302)

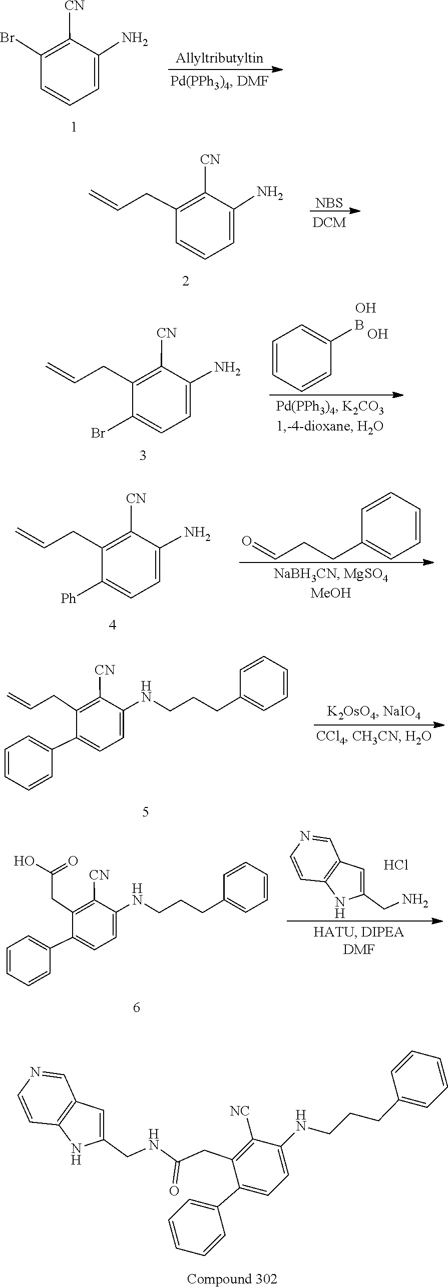

### Step 1: 2-Allyl-6-aminobenzonitrile (2)

To a mixture of 2-amino-6-bromobenzonitrile (1 g, 5.08 mmol) and Allyltributyltin (2 g, 6.04 mmol) in DMF (12 mL) was added Pd(PPh₃)₄ (586 mg, 0.51 mmol) under N₂ atmosphere. The mixture was degassed under N₂ atmosphere for three times and stirred under N₂ atmosphere at 90° C. for 16 hours. The mixture was diluted with EtOAc, washed with saturated aq. KF solution and brine, dried over anhydrous Na₂SO₄, filtered, and concentrated to dryness. The residue was purified by flash chromatography (silica gel, 0-9% EtOAc in PE) to give the title compound (600 mg, 74.7% yield) as a yellow solid. ¹H NMR (400 MHz, CDCl₃) δ 7.23 (d, J=7.9 Hz, 1H), 6.61 (t, J=7.8 Hz, 2H), 6.00-5.87 (m, 1H), 5.18-5.05 (m, 2H), 4.40 (s, 2H), 3.49 (d, J=6.6 Hz, 2H).

### Step 2: 2-Allyl-6-amino-3-bromobenzonitrile (3)

To a solution of 2-allyl-6-aminobenzonitrile (540 mg, 3.41 mmol) in DCM (3 mL) was added NBS (638 mg, 3.58 mmol), the mixture was degassed under N₂ atmosphere and stirred under N₂ atmosphere at 25° C. overnight. The mixture was diluted with EtOAc, washed with brine, dried over anhydrous Na₂SO₄, filtered, and concentrated to dryness. The residue was purified by flash chromatography (silica gel, 0-5% EtOAc in PE) to give the title compound (550 mg, yield 68.0%) as a white solid. LC/MS (ESI) m/z: 237 (M+H)⁺.

### Step 3: 2-Allyl-4-amino-[1,1′-biphenyl]-3-carbonitrile (4)

To a mixture of 2-allyl-6-amino-3-bromobenzonitrile (550 mg, 2.32 mmol), phenylboronic acid (339 mg, 2.78 mmol), and K₂CO₃ (962 mg, 6.96 mmol) in 1,4-dioxane (8 mL) and water (4 mL) was added Pd(PPh₃)₄ (268 mg, 0.23 mmol) under N₂ atmosphere. The mixture was degassed under N₂ atmosphere for three times and stirred under N₂ atmosphere at 80° C. for 16 hours. The mixture was diluted with EtOAc, washed with water and brine, dried over anhydrous Na₂SO₄, filtered, and concentrated to dryness. The residue was purified by flash chromatography (silica gel, 0-13% EtOAc in PE) to give the title compound (400 mg, yield 73.6%) as a brown solid. LC/MS (ESI) m/z: 235 (M+H)⁺.

### Step 4: 2-Allyl-4-((3-phenylpropyl)amino)-[1,1′-biphenyl]-3-carbonitrile (5)

To a mixture of 2-allyl-4-amino-[1,1′-biphenyl]-3-carbonitrile (400 mg, 1.71 mmol), NaBH₃CN (107 mg, 1.71 mmol) and MgSO₄ (205 mg, 1.71 mmol) in MeOH (6 mL) was added 3-phenylpropanal (687 mg, 5.12 mmol). The mixture was stirred at 50° C. for 16 hours. The mixture was filtered, and the filtrate was concentrated to dryness. The residue was purified by flash chromatography (silica gel, 0-13% EtOAc in PE) to give the title compound (100 mg, yield 16.6%) as a brown solid. LC/MS (ESI) m/z: 353 (M+H)⁺.

### Step 5: 2-(3-Cyano-4-((3-phenylpropyl)amino)-[1,1′-biphenyl]-2-yl)acetic acid (6)

To a solution of 2-allyl-4-((3-phenylpropyl)amino)-[1,1′-biphenyl]-3-carbonitrile (100 mg, 0.28 mmol) in CCl₄ (2 mL), CH₃CN (2 mL) and water (2 mL) was added K₂OsO₄ (10 mg, 0.03 mmol). The mixture was stirred at room temperature for 0.5 hour. NaIO₄ (306 mg, 1.42 mmol) was added to the mixture and the mixture was stirred at room temperature for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (40 mg, yield 38.1%) as a white solid. LC/MS (ESI) (m/z): 371 (M+H)⁺.

### Step 6: N-((1H-pyrrolo[3,2-c]pyridine-2-yl)methyl)-2-(3-cyano-4-((3-phenylpropyl) amino)-[1,1′-biphenyl]-2-yl)acetamide (Compound 302)

To a mixture of 2-(3-cyano-4-((3-phenylpropyl)amino)-[1,1′-biphenyl]-2-yl)acetic acid (40 mg, 0.11 mmol) and (1H-pyrrolo[3,2-c]pyridine-2-yl)methanamine hydrochloride (24 mg, 0.13 mmol) in DMF (2 mL) was added DIPEA (70 mg, 0.54 mmol) and HATU (49 mg, 0.13 mmol) at 0° C., the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 302 (1.3 mg, yield 2.4%) as a white solid. ¹H NMR (400 MHz, DMSO-d₆) δ 11.32 (s, 1H), 8.73 (s, 1H), 8.48 (t, J=5.5 Hz, 1H), 8.11 (d, J=5.8 Hz, 1H), 7.31 (t, J=5.7 Hz, 5H), 7.28-7.25 (m, 3H), 7.24-7.18 (m, 4H), 6.71 (d, J=8.8 Hz, 1H), 6.37 (s, 1H), 6.13 (t, J=5.4 Hz, 1H), 4.43 (d, J=5.4 Hz, 2H), 3.56 (s, 2H), 3.24 (d, J=6.3 Hz, 2H), 2.68 (t, J=7.6 Hz, 2H), 1.92-1.87 (m, 2H). LC/MS (ESI) (m/z): 500 (M+H)⁺. RT (Method A): 1.91 min.

### Scheme 97. Synthesis of N-((1H-pyrrolo[3,2-c]pyridine-2-yl)methyl)-2-(6-oxo-2-phenyl-5-(((4-phenylcyclohexyl)methyl)amino)pyrimidin-1(6H)-yl)acetamide (Compound 303)

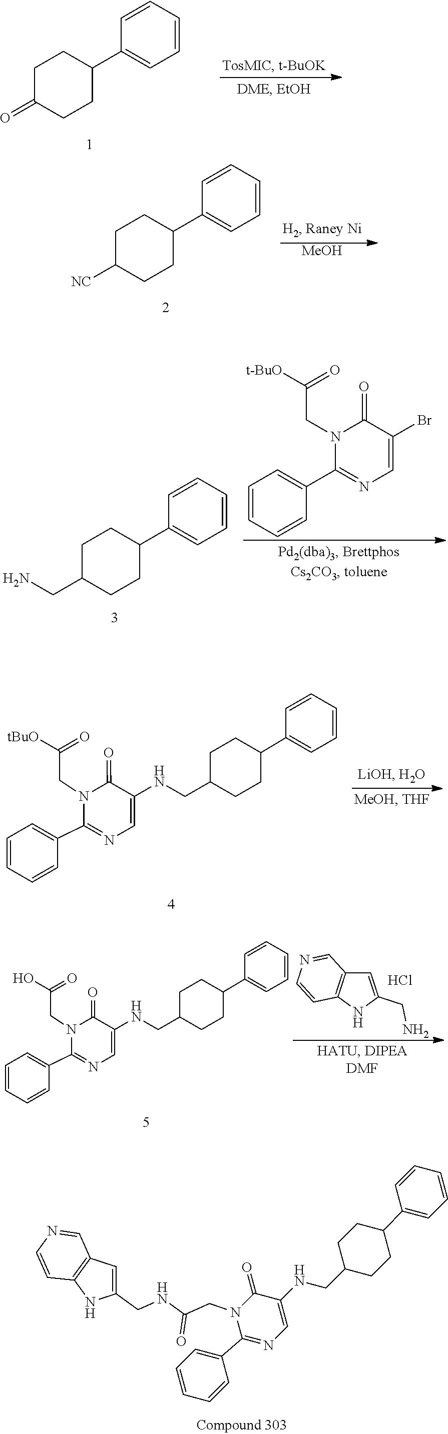

### Step 1: 4-Phenylcyclohexane-1-carbonitrile (2)

To a solution of 4-phenylcyclohexan-1-one (500 mg, 2.87 mmol) in DME (15 mL) and EtOH (0.5 mL) was added TosMIC (840 mg, 4.30 mmol), and t-BuOK (644 mg, 5.74 mmol) at 0° C. The mixture was stirred at room temperature for 16 hours. The reaction mixture was quenched with ice water and extracted with EtOAc, dried over anhydrous Na₂SO₄, filtered, and concentrated under reduced pressure to dryness. The residue was purified by flash chromatography (silica gel, 0-11% EtOAc in PE) to give title compound (220 mg, yield 41.4%) as a colorless oil. ¹H NMR (400 MHz, CDCl₃) δ 7.35-7.28 (m, 2H), 7.25-7.14 (m, 3H), 3.03 (t, J=4.8 Hz, 1H), 2.60-2.44 (m, 2H), 2.30-2.21 (m, 1H), 2.13 (d, J=13.9 Hz, 1H), 1.94-1.79 (m, 2H), 1.78-1.64 (m, 2H), 1.53-1.40 (m, 1H).

### Step 2: (4-Phenylcyclohexyl)methanamine (3)

To a solution of 4-phenylcyclohexane-1-carbonitrile (100 mg, 0.54 mmol) in MeOH (3 mL) was added Raney Ni (20 mg), the mixture was degassed under N₂ atmosphere for three times and stirred under a H₂ balloon at room temperature overnight. The mixture was filtered, and the filtrate was concentrated to dryness to give the title compound (90 mg, yield 88.1%) as a white solid, which was used directly in the next step without further purification. LC/MS (ESI) m/z: 190 (M+H)⁺.

### Step 3: Tert-butyl 2-(6-oxo-2-phenyl-5-(((4-phenylcyclohexyl)methyl)amino) pyrimidin-1(6H)-yl)acetate (4)

To a mixture of (4-phenylcyclohexyl)methanamine (90 mg, 0.48 mmol), tert-butyl 2-(5-bromo-6-oxo-2-phenylpyrimidin-1(6H)-yl)acetate (208 mg, 0.57 mmol) and Cs₂CO₃ (465 mg, 1.43 mmol) in toluene (5 mL) was added BINAP (30 mg, 0.05 mmol) and Pd₂(dba)₃ (44 mg, 0.05 mmol) under N₂ atmosphere. The mixture was degassed under N₂ atmosphere for three times and stirred under N₂ atmosphere at 110° C. for 16 hours. The mixture was diluted with EtOAc, washed with water and brine, dried over anhydrous Na₂SO₄, filtered, and concentrated to dryness. The residue was purified by flash chromatography (silica gel, 0-21% EtOAc in PE) to give title compound (90 mg, 40.0%) as a yellow oil. LC/MS (ESI) m/z: 474 (M+H)⁺.

### Step 4: 2-(6-Oxo-2-phenyl-5-(((4-phenylcyclohexyl)methyl)amino)pyrimidin-1(6H)-yl)acetic acid (5)

To a solution of tert-butyl 2-(6-oxo-2-phenyl-5-(((4-phenylcyclohexyl)methyl)amino) pyrimidin-1(6H)-yl)acetate (90 mg, 0.19 mmol) in MeOH (2 mL), THF (2 mL), and water (1 mL) was added LiOH (18 mg, 0.75 mmol) and the mixture was stirred at 60° C. for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (50 mg, yield 63.0%) as a white solid, which was used directly in the next step without further purification. LC/MS (ESI) m/z: 418 (M+H)⁺.

### Step 5: N-((1H-pyrrolo[3,2-c]pyridine-2-yl)methyl)-2-(6-oxo-2-phenyl-5-(((4-phenylcyclohexyl)methyl)amino)pyrimidin-1(6H)-yl)acetamide (Compound 303)

To a mixture of 2-(6-oxo-2-phenyl-5-(((4-phenylcyclohexyl)methyl)amino)pyrimidin-1(6H)-yl)acetic acid (50 mg, 0.11 mmol) and (1H-pyrrolo[3,2-c]pyridine-2-yl)methanamine hydrochloride (26 mg, 0.14 mmol) in DMF (2 mL) was added DIPEA (77 mg, 0.60 mmol) and HATU (55 mg, 0.14 mmol) at 0° C., the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 303 (5.6 mg, yield 8.6%) as a white solid. ¹H NMR (400 MHz, DMSO-d₆) δ 11.36 (s, 1H), 8.75 (s, 1H), 8.69 (t, J=5.2 Hz, 1H), 8.14 (d, J=5.8 Hz, 1H), 7.46 (t, J=6.0 Hz, 3H), 7.43-7.37 (m, 2H), 7.34 (d, J=5.6 Hz, 1H), 7.30-7.25 (m, 3H), 7.25-7.14 (m, 3H), 6.32 (s, 1H), 5.52-5.43 (m, 1H), 4.51 (s, 2H), 4.42 (d, J=5.3 Hz, 2H), 3.25-3.18 (m, 1H), 3.01 (t, J=6.2 Hz, 1H), 2.62-2.52 (m, 1H), 1.91 (d, J=12.9 Hz, 1H), 1.83 (d, J=12.2 Hz, 1H), 1.78-1.55 (m, 5H), 1.52-1.38 (m, 1H), 1.22-1.05 (m, 1H). LC/MS (ESI) (m/z): 547 (M+H)⁺. RT (Method A): 1.91 min.

### Scheme 98. Synthesis of (R)N-((1H-pyrrolo[3,2-c]pyridine-2-yl)methyl)-2-(5-((1-(2′-fluoro-[1,1′-biphenyl]-4-yl)ethyl)amino)-2-(2-fluorophenyl)-6-oxopyrimidin-1(6H)-yl)acetamide (Compound 308)

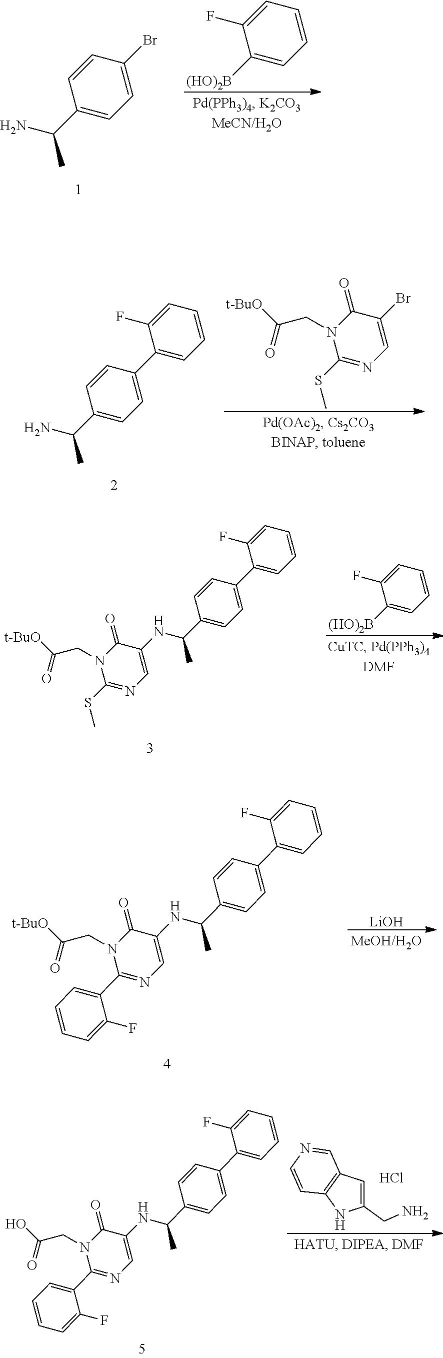

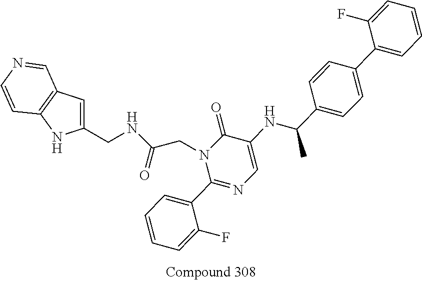 [SMILES: C[C@@H](Nc1cnc(-c2ccccc2F)n(CC(=O)NCc2cc3cnccc3[nH]2)c1=O)c1ccc(-c2ccccc2F)cc1]

### Step 1: (R)-1-(2′-fluoro-[1,1′-biphenyl]-4-yl)ethan-1-amine (2)

To a mixture of (R)-1-(4-bromophenyl)ethan-1-amine (3 g, 15 mmol) and (2-fluorophenyl)boronic acid (2.7 g, 19.5 mmol) in MeCN/water (40 mL, v/v=3/1) was added K₂CO₃ (4.1 g, 30 mmol) and Pd(PPh₃)₄ (1.7 g, 1.5 mmol). The mixture was degassed under N₂ atmosphere for three times and stirred under N₂ atmosphere at 80° C. for 4 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give the title compound (3.0 g, yield 92.7%) as a yellow solid. LC/MS (ESI) m/z: 199 (M−NH₂+H)⁺.

### Step 2: Tert-butyl (R)-2-(5-((1-(2′-fluoro-[1,1′-biphenyl]-4-yl)ethyl)amino)-2-(methylthio)-6-oxopyrimidin-1(6H)-yl)acetate (3)

To a mixture of (R)-1-(2′-fluoro-[1,1′-biphenyl]-4-yl)ethan-1-amine (1 g, 4.6 mmol) and tert-butyl 2-(5-bromo-2-(methylthio)-6-oxopyrimidin-1(6H)-yl)acetate (1.7 g, 5.1 mmol) in toluene (20 mL) was added Cs₂CO₃ (2.5 g, 7.7 mmol), BINAP (573 mg, 0.92 mmol) and Pd(OAc)₂ (100 mg, 0.46 mmol). The mixture was degassed under N₂ atmosphere for three times and stirred under N₂ atmosphere at 120° C. for 6 hours. The mixture was diluted with EtOAc, washed with water and brine, dried over anhydrous Na₂SO₄, filtered, and concentrated under reduced pressure to dryness. The residue was purified by flash chromatography (silica gel, 0-15% EtOAc in PE) to give the title compound (1.6 g, yield 66.7%) as a yellow solid. LC/MS (ESI) m/z: 470 (M+H)⁺.

### Step 3: Tert-butyl (R)-2-(5-((1-(2′-fluoro-[1,1′-biphenyl]-4-yl)ethyl)amino)-2-(2-fluorophenyl)-6-oxopyrimidin-1(6H)-yl)acetate (4)

To a mixture of tert-butyl (R)-2-(5-((1-(2′-fluoro-[1,1′-biphenyl]-4-yl)ethyl)amino)-2-(methylthio)-6-oxopyrimidin-1(6H)-yl)acetate (400 mg, 0.86 mmol) and (2-fluorophenyl)boronic acid (300 mg, 2.1 mmol) in DMF (6 mL) was added CuTc (361 mg, 1.9 mmol) and Pd(PPh₃)₄ (100 mg, 0.086 mmol). The mixture was degassed under N₂ atmosphere for three times and stirred under N₂ atmosphere at 80° C. for 16 hours. The mixture was diluted with EtOAc, washed with saturated aq. NaHCO₃ solution twice, dried over anhydrous Na₂SO₄, filtered, and concentrated under reduced pressure to dryness. The residue was purified by flash chromatography (silica gel, 0-30% EtOAc in PE) to give the title compound (200 mg, yield 45.0%) as a yellow solid. LC/MS (ESI) m/z: 518 (M+H)⁺.

### Step 4: (R)-2-(5-((1-(2′-fluoro-[1,1′-biphenyl]-4-yl)ethyl)amino)-2-(2-fluorophenyl)-6-oxopyrimidin-1(6H)-yl)acetic acid (5)

To a solution of tert-butyl (R)-2-(5-((1-(2′-fluoro-[1,1′-biphenyl]-4-yl)ethyl)amino)-2-(2-fluorophenyl)-6-oxopyrimidin-1(6H)-yl)acetate (200 mg, 0.387 mmol) in MeOH/water (4 mL, v/v=1/1) was added LiOH·H₂O (126 mg, 3 mmol) and the reaction mixture was stirred at 70° C. for 16 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (160 mg, yield 89.7%) as a yellow solid, which was used directly in the next step without further purification. LC/MS (ESI) m/z: 462 (M+H)⁺.

### Step 5: (R)—N-((1H-pyrrolo[3,2-c]pyridine-2-yl)methyl)-2-(5-((1-(2′-fluoro-[1,1′-biphenyl]-4-yl)ethyl)amino)-2-(2-fluorophenyl)-6-oxopyrimidin-1(6H)-yl)acetamide (Compound 308)

To a solution of (R)-2-(5-((1-(2′-fluoro-[1,1′-biphenyl]-4-yl)ethyl)amino)-2-(2-fluorophenyl)-6-oxopyrimidin-1(6H)-yl)acetic acid (160 mg, 0.35 mmol) in DMF (2 mL) was added (1H-pyrrolo[3,2-c]pyridine-2-yl)methanamine hydrochloride (129 mg, 0.7 mmol), HATU (266 mg, 0.7 mmol) and DIPEA (361 mg, 2.8 mmol) under N₂ atmosphere and the reaction mixture was stirred at room temperature for 10 minutes. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) and further purified by prep-HPLC to give Compound 308 (26 mg, yield 12.6%) as a white solid. ¹H NMR (400 MHz, CD₃OD) δ 8.69 (s, 1H), 8.10 (d, J=5.8 Hz, 1H), 7.52 (d, J=7.8 Hz, 2H), 7.46 (dd, J=12.0, 7.6 Hz, 4H), 7.36 (dd, J=12.8, 5.7 Hz, 3H), 7.25-7.09 (m, 4H), 6.90 (s, 1H), 6.41 (s, 1H), 5.00 (s, 1H), 4.56 (d, J=6.7 Hz, 1H), 4.46 (s, 2H), 4.33 (s, 1H), 1.63 (d, J=6.6 Hz, 3H). LC/MS (ESI) m/z: 591 (M+H)⁺. RT (Method A): 1.74 min.

### Scheme 99. Synthesis of (R)—N-((3-chloro-1H-pyrrolo[3,2-c]pyridin-2-yl)methyl)-2-(3-((1-(dibenzo[b,d]furan-2-yl)ethyl)amino)-6-(2-fluorophenyl)-2-oxopyrazin-1(2H)-yl)acetamide (Compound 351)

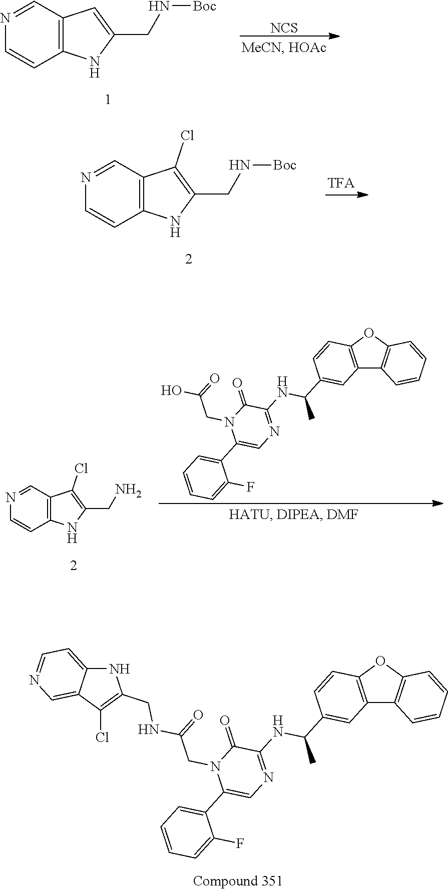 [SMILES: CC(C)(C)OC(=O)NCc1cc2cnccc2[nH]1 ; "NCS" ; CC(C)(C)OC(=O)NCc1[nH]c2ccncc2c1Cl ; NCc1[nH]c2ccncc2c1Cl ; C[C@@H](Nc1ncc(-c2ccccc2F)n(CC(=O)O)c1=O)c1ccc2oc3ccccc3c2c1 ; C[C@@H](Nc1ncc(-c2ccccc2F)n(CC(=O)NCc2[nH]c3ccncc3c2Cl)c1=O)c1ccc2oc3ccccc3c2c1]

### Step 1: Tert-butyl ((3-chloro-1H-pyrrolo[3,2-c]pyridin-2-yl)methyl)carbamate (2)

To a mixture of tert-butyl ((1H-pyrrolo[3,2-c]pyridine-2-yl)methyl)carbamate (100 mg, 0.40 mmol) and NCS (60 mg, 0.45 mmol) in Acetonitrile (2 mL) and acetic acid (0.5 mL) and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness to give the title compound (45 mg, yield 39%) as a yellow solid. LC/MS (ESI) m/z: 282 (M+H)⁺.

### Step 2: (3-Chloro-1H-pyrrolo[3,2-c]pyridin-2-yl)methanamine (3)

A solution of tert-butyl ((3-chloro-1H-pyrrolo[3,2-c]pyridine-2-yl)methyl)carbamate (40 mg, 0.14 mmol) in TFA (1 mL) was stirred under N₂ atmosphere at room temperature for 1 hours. The reaction mixture was concentrated to dryness under reduced pressure to give the title compound (45 mg, crude) as a brown oil, which was used directly in the next step without further purification. LC/MS (ESI) m/z: 182 (M+H)⁺.

### Step 4: (R)—N-((3-chloro-1H-pyrrolo[3,2-c]pyridine-2-yl)methyl)-2-(5-((1-(dibenzo[b,d]furan-2-yl)ethyl)amino)-2-(2-fluorophenyl)-6-oxopyrimidin-1(6H)-yl)acetamide (Compound 351)

To a mixture of I-2-(5-((1-(dibenzo[b,d]furan-2-yl)ethyl)amino)-2-(2-fluorophenyl)-6-oxopyrimidin-1(6H)-yl)acetic acid (50 mg, 0.11 mmol) and (3-chloro-1H-pyrrolo[3,2-c]pyridine-2-yl)methanamine (22 mg, 0.12 mmol) in DMF (2 mL) was added DIPEA (70 mg, 0.55 mmol) and HATU (50 mg, 0.13 mmol) and the mixture was stirred at room temperature for 1 hour. The mixture was quenched with water and extracted with EtOAc. The combined organic layers were washed with brine, dried over anhydrous Na₂SO₄, filtered, and concentrated under reduced pressure to dryness. The residue was purified by prep-TLC (DCM:MeOH=8:1) and further purified by prep-HPLC to give Compound 351 (15 mg, yield 22%) as a white solid. ¹H NMR (400 MHz, DMSO-d₆) δ 11.57 (s, 1H), 8.72 (s, 1H), 8.60 (s, 1H), 8.23 (s, 2H), 8.11 (d, J=8.1 Hz, 1H), 7.72-7.61 (m, 3H), 7.52 (t, J=7.4 Hz, 1H), 7.46-7.37 (m, 3H), 7.25 (dd, J=22.2, 12.6 Hz, 2H), 7.02 (m, 2H), 6.03 (d, J=6.5 Hz, 1H), 4.87-4.67 and 4.28-3.99 (m, 1H), 4.68 (d, J=6.6 Hz, 1H), 4.33 (s, 2H), 1.61 (d, J=6.5 Hz, 3H). LC/MS (ESI) (m/z): 621 (M+H)⁺. RT (Method A): 1.78 min.

### Scheme 100. Synthesis of (R)—N-((1H-pyrrolo[3,2-c]pyridine-2-yl)methyl)-2-(5-((1-(dibenzo[b,d]furan-3-yl)ethyl)amino)-2-(2-fluorophenyl)-6-oxopyrimidin-1(6H)-yl)acetamide

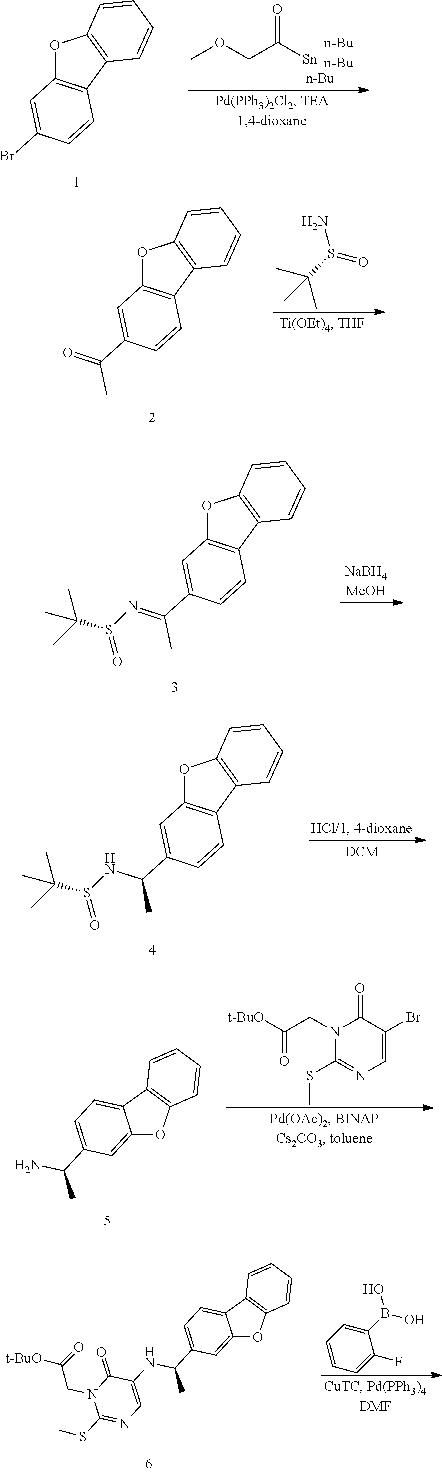

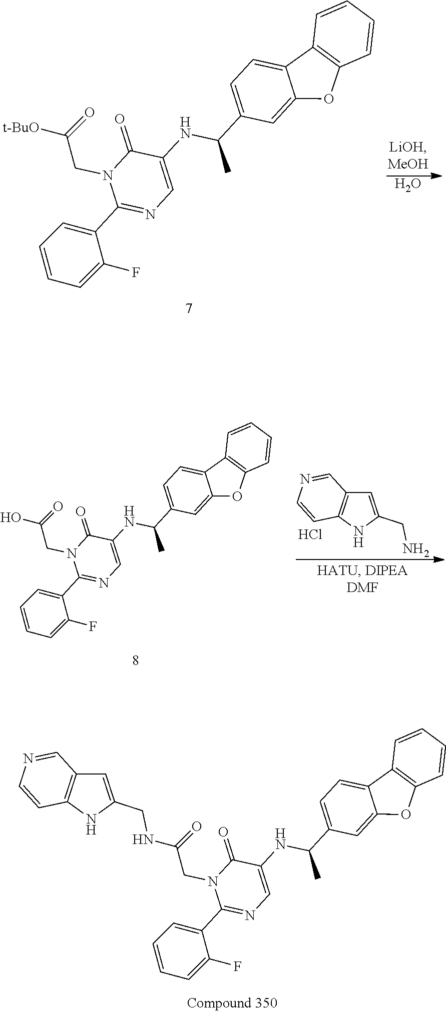

### Step 1: 1-(Dibenzo[b,d]furan-3-yl)ethan-1-one (2)

To a mixture of 3-bromodibenzo[b,d]furan (5.0 g, 20.24 mmol) and ethyl tributylstannanecarboxylate (8.8 g, 24.28 mmol) in 1,4-dioxane (100 mL) was added Pd(PPh₃)₂Cl₂ (1.4 g, 2.02 mmol), TEA (5.1 g, 50.59 mmol) under N₂ atmosphere, the reaction mixture was degassed under N₂ atmosphere for three times and stirred at 100° C. for 3 hours. The mixture was diluted with water and extracted with EtOAc twice. The combined organic layers were washed with saturated aq. KF solution and brine, dried over anhydrous Na₂SO₄, filtered, and concentrated under reduced pressure to dryness. The residue was purified by flash chromatography (silica gel, 0-5% EtOAc in PE) to give the title compound (4.00 g, yield 94.1%) as a yellow solid. LC/MS (ESI) m/z: 211 (M+H)⁺.

### Step 2: (R,E)-N-(1-(dibenzo[b,d]furan-3-yl)ethylidene)-2-methylpropane-2-sulfinamide (3)

To a mixture of 1-(dibenzo[b,d]furan-3-yl)ethan-1-one (4.00 g, 19.00 mmol) and (R)-2-methylpropane-2-sulfinamide (5.76 g, 47.60 mmol) in THF (100 mL) was added Ti(OEt)₄ (17.4 g, 7.62 mmol) under N₂ atmosphere and the reaction mixture was stirred at 85° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-8% EtOAc in PE) to give the title compound (5.5 g, yield 93.2%) as a yellow solid. LC/MS (ESI) m/z: 314 (M+H)⁺.

### Step 3: (R)—N—((R)-1-(dibenzo[b,d]furan-3-yl)ethyl)-2-methylpropane-2-sulfinamide (4)

To a solution of (R,E)-N-(1-(dibenzo[b,d]furan-3-yl)ethylidene)-2-methylpropane-2-sulfinamide (5.5 g, 17.57 mmol) in MeOH (60 mL) was added NaBH₄ (1.65 g, 43.42 mmol) in portions at 0° C. and the mixture was stirred at room temperature for 3 hours. The mixture was quenched with saturated aq. NH₄Cl solution at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (2.1 g, yield 37.9%) as a yellow solid. LC/MS (ESI) m/z: 316 (M+H)⁺.

### Step 4: (R)-1-(dibenzo[b,d]furan-3-yl)ethan-1-amine (5)

To a solution of N—((R)-1-(dibenzo[b,d]furan-3-yl)ethyl)-2-methylpropane-2-sulfinamide (2.1 g, 2.07 mmol) in DCM (30 mL) was added HCl/1,4-dioxane (20 mL, 4M) and the reaction mixture was stirred under N₂ atmosphere at room temperature for 2 hours. The reaction mixture was concentrated under reduced pressure to dryness to give the title compound (1.4 g, yield 99.5%) as a white solid, which was used directly in the next step without further purification. LC/MS (ESI) m/z: 212 (M+H)⁺.

### Step 5: Tert-butyl (R)-2-(5-((1-(dibenzo[b,d]furan-3-yl)ethyl)amino)-2-(methylthio)-6-oxopyrimidin-1(6H)-yl)acetate (6)

To a mixture of I-1-(dibenzo[b,d]furan-3-yl)ethan-1-amine (600 mg, 2.84 mmol) and tert-butyl 2-(5-bromo-2-(methylthio)-6-oxopyrimidin-1(6H)-yl)acetate (475 mg, 1.42 mmol) in toluene (6 mL) was added Cs₂CO₃ (898 mg, 2.76 mmol), BINAP (177 mg, 0.28 mmol), Pd(OAc)₂ (32 mg, 0.14 mmol) under N₂ atmosphere and the reaction mixture was stirred at 100° C. overnight. The mixture was diluted with EtOAc, washed with brine, dried over anhydrous Na₂SO₄, filtered, and concentrated under reduced pressure to dryness. The residue was purified by flash chromatography (silica gel, 0-10% EtOAc in PE) to give the title compound (264 mg, yield 39.9%) as orange solid. LC/MS (ESI) m/z: 466 (M+H)⁺.

### Step 6: Tert-butyl (R)-2-(5-((1-(dibenzo[b,d]furan-3-yl)ethyl)amino)-2-(2-fluorophenyl)-6-oxopyrimidin-1(6H)-yl)acetate (7)

To a mixture of tert-butyl (R)-2-(5-((1-(dibenzo[b,d]furan-3-yl)ethyl)amino)-2-(methylthio)-6-oxopyrimidin-1(6H)-yl)acetate (260 mg, 0.60 mmol) and (2-fluorophenyl)boronic acid (157 mg, 1.12 mmol) in DMF (5 mL) was added CuTc (235 mg, 1.23 mmol) and Pd(PPh₃)₄ (64.5 mg, 0.06 mmol) under N₂ atmosphere. The mixture was degassed under N₂ atmosphere for three times and stirred at 80° C. overnight in a sealed tube. The reaction mixture was diluted with EtOAc and filtered. The filtrate was washed with saturated aq. NaHCO₃ solution and brine, dried over anhydrous Na₂SO₄, filtered, and concentrated under reduced pressure to dryness. The residue was purified by flash chromatography (silica gel, 0-15% EtOAc in PE) to give the title compound (200 mg, yield 69.7%) as a yellow solid. LC/MS (ESI) m/z: 514 (M+H)⁺.

### Step 7: (R)-2-(5-((1-(dibenzo[b,d]furan-3-yl)ethyl)amino)-2-(2-fluorophenyl)-6-oxopyrimidin-1(6H)-yl)acetic acid (8)

To a solution of tert-butyl (R)-2-(5-((1-(dibenzo[b,d]furan-3-yl)ethyl)amino)-2-(2-fluorophenyl)-6-oxopyrimidin-1(6H)-yl)acetate (200 mg, 0.40 mmol) in MeOH/water (9 mL, v/v=2/1) was added LiOH (38 mg, 1.58 mmol) and the mixture was stirred at 60° C. for 3 hours. The mixture was acidified with 1 N aq. HCl to pH˜5 and extracted with EtOAc twice. The combined organic layers were washed with brine, dried over anhydrous Na₂SO₄, filtered, and concentrated under reduced pressure to dryness to give the title compound (170 mg, yield 95.5%) as a yellow solid, which was used directly in the next step without further purification. LC/MS (ESI) m/z: 458 (M+H)⁺.

### Step 8: (R)—N-((1H-pyrrolo[3,2-c]pyridine-2-yl)methyl)-2-(5-((1-(dibenzo[b,d]furan-3-yl)ethyl)amino)-2-(2-fluorophenyl)-6-oxopyrimidin-1(6H)-yl)acetamide (Compound 350)

To a mixture of (R)-2-(5-((1-(dibenzo[b,d]furan-3-yl)ethyl)amino)-2-(2-fluorophenyl)-6-oxopyrimidin-1(6H)-yl)acetic acid (170 mg, 0.37 mmol) and (1H-pyrrolo[3,2-c]pyridine-2-yl)methanamine hydrochloride (152 mg, 0.83 mmol) in DMF (4 mL) was added DIPEA (282 mg, 2.19 mmol) and HATU (250 mg, 0.66 mmol) under N₂ atmosphere and the reaction mixture was stirred at room temperature for 1 hour. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-15% MeOH in DCM) and further purified by prep-HPLC to give Compound 350 (24.2 mg, yield 11.1%) as a white solid. ¹H NMR (400 MHz, CD₃OD) δ 8.94 (s, 1H), 8.44 (s, 1H), 8.25 (d, J=6.3 Hz, 1H), 7.99 (d, J=7.8 Hz, 2H), 7.74 (d, J=6.5 Hz, 1H), 7.63 (s, 1H), 7.55 (d, J=8.4 Hz, 1H), 7.48-7.42 (m, 3H), 7.34 (t, J=7.1 Hz, 2H), 7.18 (t, J=9.0 Hz, 1H), 7.11 (s, 1H), 6.92 (s, 1H), 6.71 (s, 1H), 4.98 (s, 1H), 4.71-4.67 (m, 1H), 4.53 (s, 2H), 4.36 (s, 1H), 1.67 (d, J=6.6 Hz, 3H). LC/MS (ESI) m/z: 587 (M+H)⁺. RT (Method A): 1.73 min.

Compound 347 was prepared based on Scheme 100:

| | Reactant | Reactant B | Reactant | Reactant | Reactant | Reactant | Characterization |
|---|---|---|---|---|---|---|---|
| # | A | B | C | D | E | F | Data |
| 347^{a} | 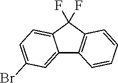 | 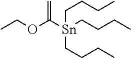 | 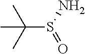 | 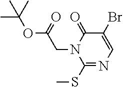 | 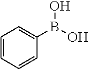 | 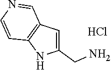 | ¹H NMR (400 MHz, DMSO-d₆) δ 11.32 (s, 1H), 8.75 (s, 1H), 8.68 (m, 1H), 8.12 (m, 1H), 7.96 (s, 1H), |
| | | | | | | | 7.80 (d, J = 7.6 |
| | | | | | | | Hz, 1H), 7.69 (t, |
| | | | | | | | J = 7.3 Hz, 2H), |
| | | | | | | | 7.59 (t, J = 7.1 Hz, |
| | | | | | | | 1H), 7.51 (d, J = |
| | | | | | | | 7.5 Hz, 1H), 7.41 |
| | | | | | | | (m, 4H), 7.35 (m, |
| | | | | | | | 3H), 6.97 (s, 1H), |
| | | | | | | | 6.32 (s, 1H), 5.96 |
| | | | | | | | (d, J = 6.4 Hz, |
| | | | | | | | 1H), 4.62-4.51 |
| | | | | | | | (m, 2H), 4.43 (m, |
| | | | | | | | 3H), 1.57 (d, J = |
| | | | | | | | 5.7 Hz, 3H). |
| | | | | | | | LC/MS (ESI) m/z: |
| | | | | | | | 603 (M + H)⁺. RT |
| | | | | | | | (Method A): 1.74 |
| | | | | | | | min. |

^{a}step 3 was performed with DIBAL—H instead of NaBH₄, and Pd₂(dba)₃ was used in place of Pd(OAc)₂ in Step 5.

### Scheme 101. Synthesis of N-((1H-pyrrolo[3,2-c]pyridine-2-yl)methyl)-2-(5-((2-(dibenzo[b,d]furan-2-yl)propan-2-yl)amino)-2-(2-fluorophenyl)-6-oxopyrimidin-1(6H)-yl)acetamide (Compound 311)

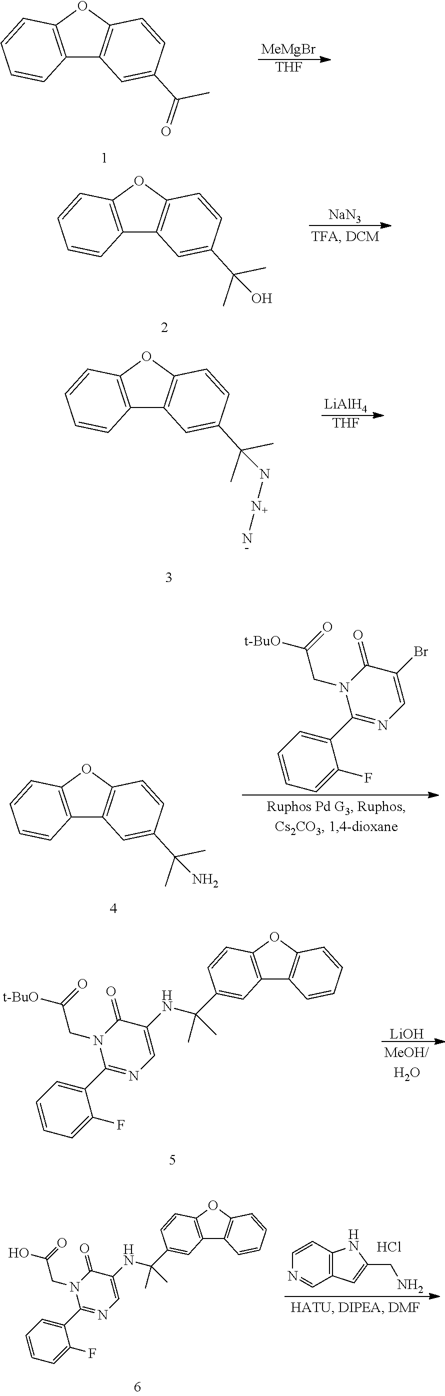

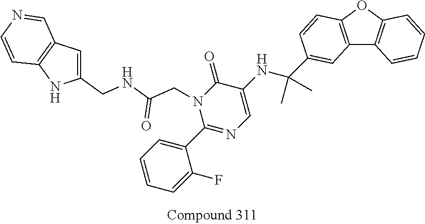 [SMILES: CC(C)(Nc1cnc(-c2ccccc2F)n(CC(=O)NCc2cc3cnccc3[nH]2)c1=O)c1ccc2oc3ccccc3c2c1]

### Step 1: 2-(Dibenzo[b,d]furan-2-yl)propan-2-ol (2)

To a solution of 1-(dibenzo[b,d]furan-2-yl)ethan-1-one (1.0 g, 4.75 mmol) in THF (10 mL) was added MeMgBr/THF solution (4.8 mL, 4.8 mmol, 1 M) at 0° C. under N₂ atmosphere and the reaction mixture was stirred at room temperature for 3 hours. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (800 mg, yield 74.3%) as a yellow oil. LC/MS (ESI) m/z: 209 (M+H)⁺.

### Step 2: 2-(2-Azidopropan-2-yl)dibenzo[b,d]furan (3)

To a solution of 2-(dibenzo[b,d]furan-2-yl)propan-2-ol (800 mg, 3.53 mmol) in DCM (10 mL) was added NaN₃ (506 mg, 7.78 mmol) in portions at 0° C. followed by dropwise addition of TFA/DCM (4.0 mL, v/v=2/1) under N₂ atmosphere, the reaction mixture was stirred at room temperature for 2 hours. The mixture was diluted with DCM, washed with brine, dried over anhydrous Na₂SO₄, filtered, and concentrated under reduced pressure to dryness. The residue was purified by flash chromatography (silica gel, 0-5% EtOAc in PE) to give the title compound (800 mg, yield 89.6%) as a colorless oil. ¹H NMR (400 MHz, CDCl₃) δ 8.04 (s, 1H), 7.98 (d, J=7.0 Hz, 1H), 7.57 (d, J=10.5 Hz, 3H), 7.47 (s, 1H), 7.38-7.34 (m, 1H), 1.75 (d, J=3.6 Hz, 6H).

### Step 3: 2-(Dibenzo[b,d]furan-2-yl)propan-2-amine (4)

To a solution of 2-(2-azidopropan-2-yl)dibenzo[b,d]furan (800 mg, 3.18 mmol) in THF (10 mL) was added LiAlH₄ (116 mg, 3.06 mmol) under N₂ atmosphere at 0° C. and the reaction mixture was stirred at room temperature for 3 hours. The mixture was quenched with saturated aq. NaOH solution at 0° C. and extracted with DCM twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (350 mg, yield 48.8%) as a yellow oil. LC/MS (ESI) m/z: 209 (M+H)⁺.

### Step 4: Tert-butyl 2-(5-((2-(dibenzo[b,d]furan-2-yl)propan-2-yl)amino)-2-(2-fluorophenyl)-6-oxopyrimidin-1(6H)-yl)acetate (5)

To a mixture of tert-butyl 2-(5-bromo-2-(2-fluorophenyl)-6-oxopyrimidin-1(6H)-yl)acetate (100 mg, 0.40 mmol) and 2-(dibenzo[b,d]furan-2-yl)propan-2-amine (95 mg, 0.38 mmol) in 1,4-dioxane (5 mL) was added Cs₂CO₃ (890 mg, 2.73 mmol), Ruphos (83 mg, 0.13 mmol), Ruphos Pd G3 (148 mg, 0.16 mmol) under N₂ atmosphere, the reaction mixture was degassed under N₂ atmosphere for three times and stirred at 100° C. for 6 hours. The mixture was diluted with EtOAc, washed with brine, dried over anhydrous Na₂SO₄, filtered, and concentrated under reduced pressure to dryness. The residue was purified by flash chromatography (silica gel, 0-30% EtOAc in PE) to give the title compound (240 mg, yield 51.2%) as a yellow solid. LC/MS (ESI) m/z: 528 (M+H)⁺.

### Step 5: 2-(5-((2-(Dibenzo[b,d]furan-2-yl)propan-2-yl)amino)-2-(2-fluorophenyl)-6-oxopyrimidin-1(6H)-yl)acetic acid (6)

To a solution of tert-butyl 2-(5-((2-(dibenzo[b,d]furan-2-yl)propan-2-yl)amino)-2-(2-fluorophenyl)-6-oxopyrimidin-1(6H)-yl)acetate (90 mg, 0.17 mmol) in MeOH/water (2.0 mL, v/v=4/1) was added LiOH·H₂O (15 mg, 0.35 mmol) and the reaction mixture was stirred at 60° C. overnight. Mixture was acidified with 1 N aq. HCl to pH˜3 and extracted with EtOAc twice. The combined organic layers were dried over anhydrous Na₂SO₄, filtered, and concentrated under reduced pressure to give the title compound (70 mg, yield 87.0%) as a yellow solid, which was used directly in the next step without further purification. LC/MS (ESI) m/z: 472 (M+H)⁺.

### Step 6: N-((1H-pyrrolo[3,2-c]pyridine-2-yl)methyl)-2-(5-((2-(dibenzo[b,d]furan-2-yl)propan-2-yl)amino)-2-(2-fluorophenyl)-6-oxopyrimidin-1(6H)-yl)acetamide (Compound 311)

To a mixture of 2-(5-((2-(dibenzo[b,d]furan-2-yl)propan-2-yl)amino)-2-(2-fluorophenyl)-6-oxopyrimidin-1(6H)-yl)acetic acid (70 mg, 0.14 mmol) and (1H-pyrrolo[3,2-c]pyridine-2-yl)methanamine hydrochloride (33 mg, 0.17 mmol) in DMF (2 mL) was added DIPEA (96 mg, 0.74 mmol) and HATU (68 mg, 0.17 mmol) and the reaction mixture was stirred at room temperature for 1 hour. The mixture was concentrated under reduced pressure to dryness. The residue was purified by prep-HPLC to give Compound 311 (6.6 mg, yield 7.4%) as pink solid. ¹H NMR (400 MHz, CD₃OD) δ 8.77 (s, 1H), 8.20 (s, 1H), 8.16 (d, J=5.2 Hz, 1H), 8.05 (d, J=7.0 Hz, 1H), 7.68 (d, J=9.0 Hz, 1H), 7.59 (d, J=8.7 Hz, 2H), 7.48 (q, J=8.1 Hz, 3H), 7.39-7.32 (m, 2H), 7.17 (t, J=9.7 Hz, 1H), 7.09 (t, J=7.9 Hz, 1H), 6.52 (d, J=8.6 Hz, 2H), 5.02-5.01 (m, 1H), 4.51 (s, 2H), 4.33-4.32 (s, 1H), 1.86 (s, 6H). LC/MS (ESI) m/z: 601 (M+H)⁺. RT (Method A): 1.82 min.

### Scheme 102. Synthesis of N-((1H-pyrrolo[3,2-c]pyridine-2-yl)methyl)-2-(5-((dibenzo[b,d]furan-2-ylmethyl)amino)-6-oxo-2-(1H-pyrazol-4-yl)pyrimidin-1(6H)-yl)acetamide (Compound 312)

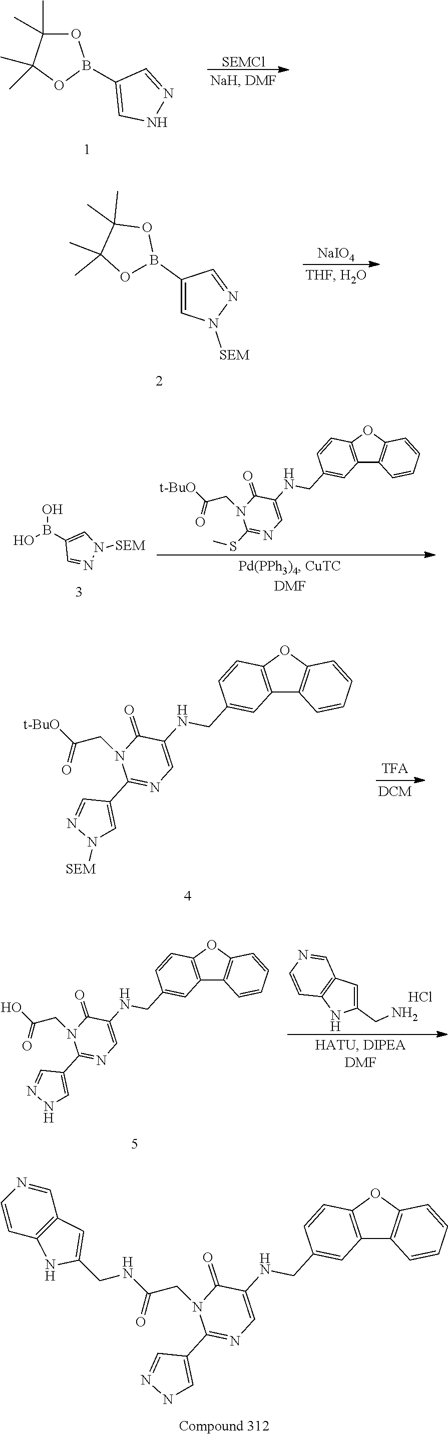

### Step 1: 4-(4,4,5,5-tetramethyl-1,3,2-dioxaborolan-2-yl)-1-((2-(trimethylsilyl)ethoxy) methyl)-1H-pyrazole (2)

To a solution of 4-(4,4,5,5-tetramethyl-1,3,2-dioxaborolan-2-yl)-1H-pyrazole (3.0 g, 15.46 mmol) in DMF (30 mL) was added NaH (930 mg, 23.25 mmol, 60% dispersion in mineral oil) at 0° C. under N₂ atmosphere and the mixture was stirred at 0° C. for 30 minutes. Then SEMCl (3.35 g, 20.10 mmol) was added into the above mixture and the resulting mixture was stirred at room temperature for 2 hours. The mixture was quenched with saturated aq.NH₄Cl solution at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-15% EtOAc in PE) to give the title compound (1.8 g, yield 36.0%) as a light oil. ¹H NMR (400 MHz, CDCl₃) δ 7.85 (s, 1H), 7.81 (s, 1H), 5.43 (s, 2H), 3.57-3.52 (m, 2H), 1.32 (s, 12H), 0.92-0.87 (m, 2H), −0.04 (s, 9H). LC/MS (ESI) m/z: 325 (M+H)⁺.

### Step 2: (1-((2-(Trimethylsilyl)ethoxy)methyl)-1H-pyrazol-4-yl)boronic acid (3)

To a solution of 4-(4,4,5,5-tetramethyl-1,3,2-dioxaborolan-2-yl)-1-((2-(trimethylsilyl) ethoxy)methyl)-1H-pyrazole (1.5 g, 4.63 mmol) in THF (12 mL) and water (3 mL) was added NaIO₄ (3.96 mg, 18.52 mmol) and the mixture was stirred at room temperature overnight. The mixture was filtered, and the filtrate was washed with brine, dried over anhydrous Na₂SO₄, filtered, and concentrated under reduced pressure to dryness to give the title compound (1.14 g, crude) as a yellow oil, which was used directly in the next step without further purification. LC/MS (ESI) m/z: 243 (M+H)⁺.

### Step 3: Tert-butyl 2-(5-((dibenzo[b,d]furan-2-ylmethyl)amino)-6-oxo-2-(1-((2-(trimethylsilyl)ethoxy)methyl)-1H-pyrazol-4-yl)pyrimidin-1(6H)-yl)acetate (4)

To a mixture of tert-butyl 2-(5-((dibenzo[b,d]furan-2-ylmethyl)amino)-2-(methylthio)-6-oxopyrimidin-1(6H)-yl)acetate (400 mg, 0.89 mmol) and (1-((2-(trimethylsilyl) ethoxy)methyl)-1H-pyrazol-4-yl)boronic acid (650 mg, 2.68 mmol) in DMF (8 mL) was added CuTC (370 mg, 1.94 mmol) and Pd(PPh₃)₄ (102 mg, 0.088 mmol) at room temperature under N₂ atmosphere, the reaction mixture was degassed under N₂ atmosphere for three times and stirred at 90° C. for 48 hours in a sealed tube. The mixture was diluted with water and extracted with EtOAc twice. The combined organic layers were washed with saturated aq. NaHCO₃ solution and brine, dried over anhydrous Na₂SO₄, filtered, and concentrated under reduced pressure to dryness. The residue was purified by flash chromatography (silica gel, 0-70% EtOAc in PE) to give the title compound (150 mg, yield 28.1%) as a yellow oil. LC/MS (ESI) m/z: 602 (M+H)⁺.

### Step 4: 2-(5-((dibenzo[b,d]furan-2-ylmethyl)amino)-6-oxo-2-(1H-pyrazol-4-yl) pyrimidin-1(6H)-yl)acetic acid (5)

To a solution of tert-butyl 2-(5-((dibenzo[b,d]furan-2-ylmethyl)amino)-6-oxo-2-(1-((2-(trimethylsilyl)ethoxy)methyl)-1H-pyrazol-4-yl)pyrimidin-1(6H)-yl)acetate (150 mg, 0.25 mmol) in DCM (2 mL) was added TFA (2 mL) and the mixture was stirred at room temperature for 3 hours. The mixture was concentrated under reduced pressure to dryness to give the title compound (60 mg, yield 58.0%) as a yellow oil, which was used directly in the next step without further purification. LC/MS (ESI) m/z: 416 (M+H)⁺.

### Step 5: N-((1H-pyrrolo[3,2-c]pyridine-2-yl)methyl)-2-(5-((dibenzo[b,d]furan-2-ylmethyl)amino)-6-oxo-2-(1H-pyrazol-4-yl)pyrimidin-1(6H)-yl)acetamide (Compound 312)

To a mixture of 2-(5-((dibenzo[b,d]furan-2-ylmethyl)amino)-6-oxo-2-(1H-pyrazol-4-yl)pyrimidin-1(6H)-yl)acetic acid (60 mg, 0.14 mmol) and (1H-pyrrolo[3,2-c]pyridine-2-yl)methanamine hydrochloride (40 mg, 0.22 mmol) in DMF (3 mL) was added DIPEA (75 mg, 0.58 mmol) and HATU (71 mg, 0.19 mmol) and the reaction mixture was stirred at room temperature for 1 hour. The mixture was concentrated under reduced pressure to dryness. The residue was purified by prep-TLC (DCM:MeOH=7:1) and further purified by prep-HPLC to give Compound 312 (6.0 mg, yield 7.6%) as a white solid. ¹H NMR (400 MHz, CD₃OD) δ 8.81 (s, 1H), 8.19-8.04 (m, 3H), 8.01-7.70 (m, 2H), 7.69-7.55 (m, 3H), 7.55-7.50 (m, 2H), 7.41 (d, J=6.8 Hz, 1H), 7.11 (d, J=18.5 Hz, 1H), 6.61 (s, 1H), 4.85 (s, 2H), 4.65-4.64 (m, 2H), 4.61 (d, J=8.9 Hz, 2H). LC/MS (ESI) m/z: 545 (M+H)⁺. RT (Method A): 1.30 min.

### Scheme 103. Synthesis of (S)—N-((1H-pyrrolo[3,2-c]pyridine-2-yl)methyl)-2-(3-((1-(dibenzo[b,d]furan-2-yl)ethyl)amino)-6-(2-fluorophenyl)-2-oxopyrazin-1(2H)-yl)acetamide (Compound 314)

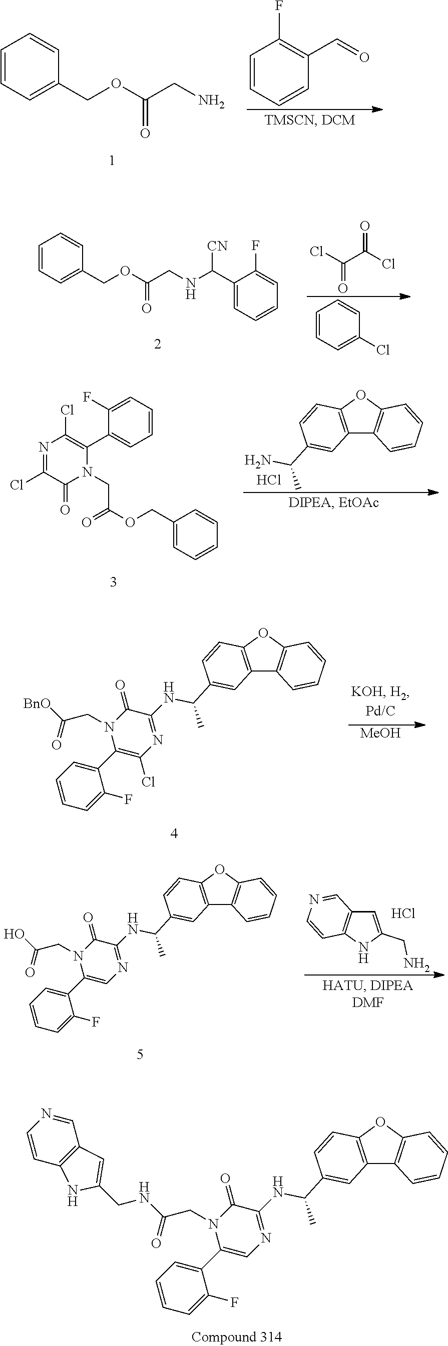

### Step 1: Benzyl (cyano(2-fluorophenyl)methyl)glycinate (2)

To a solution of benzyl glycinate (3.0 g, 18.2 mmol) in DCM (30 mL) was added 2-fluorobenzaldehyde (2.3 g, 18.2 mmol) in portions at room temperature. TMSCN (2.0 g, 20.0 mmol) was added to the mixture dropwise over 10 minutes. The mixture was stirred at room temperature for 4 hours. The mixture was concentrated under reduced pressure to dryness and the residue was purified by flash chromatography (silica gel, 0-30% EtOAc in PE) to give the title compound (3.7 g, yield 68.5%) as a yellow oil. LC/MS (ESI) m/z: 299 (M+H)⁺.

### Step 2: Benzyl 2-(3,5-dichloro-6-(2-fluorophenyl)-2-oxopyrazin-1(2H)-yl)acetate (3)

To a solution of benzyl (cyano(2-fluorophenyl)methyl)glycinate (3.0 g, 10.1 mmol) in chlorobenzene (40 mL) and HCl/1,4-dioxane (5 mL) was added oxalyl dichloride (5.1 g, 40.3 mmol) under N₂ atmosphere and the reaction mixture was stirred at 100° C. for 16 hours. The mixture was concentrated under reduced pressure to dryness. The residue was purified by flash chromatography (silica gel, 0-25% EtOAc in PE) to give the title compound (3.2 g, yield 78.2%) as a yellow solid. LC/MS (ESI) m/z: 407 (M+H)⁺.

### Step 3: Benzyl (S)-2-(5-chloro-3-((1-(dibenzo[b,d]furan-2-yl)ethyl)amino)-6-(2-fluorophenyl)-2-oxopyrazin-1(2H)-yl)acetate (4)

To a mixture of benzyl 2-(3,5-dichloro-6-(2-fluorophenyl)-2-oxopyrazin-1(2H)-yl)acetate (300 mg, 0.74 mmol) and (S)-1-(dibenzo[b,d]furan-2-yl)ethan-1-amine hydrochloride (182 mg, 0.74 mmol) in EtOAc (3 mL) was added DIPEA (286 mg, 2.2 mmol) and the mixture was stirred at 80° C. overnight. The mixture was concentrated under reduced pressure to dryness. The residue was purified by flash chromatography (silica gel, 0-30% EtOAc in PE) to give the title compound (370 mg, yield 86.2%) as a yellow solid. LC/MS (ESI) m/z: 582 (M+H)⁺.

### Step 4: (S)-2-(3-((1-(dibenzo[b,d]furan-2-yl)ethyl)amino)-6-(2-fluorophenyl)-2-oxopyrazin-1(2H)-yl)acetic acid (5)

To a solution of benzyl (S)-2-(5-chloro-3-((1-(dibenzo[b,d]furan-2-yl)ethyl)amino)-6-(2-fluorophenyl)-2-oxopyrazin-1(2H)-yl)acetate (200 mg, 0.34 mmol) in MeOH (4 mL) was added KOH (19 mg, 0.34 mmol) and Pd/C (30 mg, 10% wt.), the mixture was degassed under N₂ atmosphere for three times and stirred under a H₂ balloon at 50° C. for 5 hours. The mixture was filtered, and the filtrate was concentrated to dryness to give the title compound (150 mg, yield 95.5%) as a yellow solid, which was used directly in the next step without further purification. LC/MS (ESI) m/z: 458 (M+H)⁺.

### Step 5: (S)—N-((1H-pyrrolo[3,2-c]pyridine-2-yl)methyl)-2-(3-((1-(dibenzo[b,d]furan-2-yl)ethyl)amino)-6-(2-fluorophenyl)-2-oxopyrazin-1(2H)-yl)acetamide (Compound 314)

To a mixture of (S)-2-(3-((1-(dibenzo[b,d]furan-2-yl)ethyl)amino)-6-(2-fluorophenyl)-2-oxopyrazin-1(2H)-yl)acetic acid (150 mg, 0.33 mmol) and (1H-pyrrolo[3,2-c]pyridine-2-yl)methanamine hydrochloride (90 mg, 0.49 mmol) in DMF (3 mL) was added HATU (187 mg, 0.49 mmol) and DIPEA (212 mg, 1.6 mmol) under N₂ atmosphere and the reaction mixture was stirred at room temperature for 1 hour. The mixture was concentrated under reduced pressure to dryness. The residue was purified by flash chromatography (silica gel, 0-30% MeOH in DCM) and further purified by prep-HPLC to give the title compound (22.9 mg, yield 11.9%) as a white solid. ¹HNMR (400 MHz, 00300) δ 8.69 (s, 1H), 8.09 (d, J=6.2 Hz, 2H), 8.01 (d, J=6.6 Hz, 1H), 7.53 (d, J=10.3 Hz, 3H), 7.48-7.39 (m, 2H), 7.37-7.25 (m, 3H), 7.15 (s, 1H), 7.04 (s, 1H), 6.70 (s, 1H), 6.39 (s, 1H), 5.34 (d, J=6.9 Hz, 1H), 4.84 (s, 1H), 4.43 (s, 2H), 4.22 (s, 1H), 1.70 (d, J=6.7 Hz, 3H). LC/MS (ESI) m/z: 587 (M+H)⁺. RT (Method A): 1.84 min.

Compounds 316, 348, and 349 were prepared based on Scheme 103:

| | Reactant | Reactant | Reactant | Reactant | Reactant | Characterization |
|---|---|---|---|---|---|---|
| # | A | B | C | D | E | Data |
| 316 | 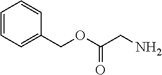 | 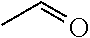 | 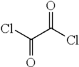 | 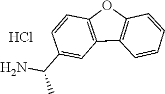 | 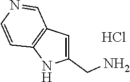 | ¹H-NMR (400 MHz, CD₃OD) δ 8.73 (s, 1H), 8.10 (d, J = 6.0 Hz, 1H), 8.04 (s, 1H), 7.98 (d, J = 7.9 Hz, 1H), 7.55 (d, J = 8.2 Hz, 1H), 7.51 (s, 2H), 7.47 (d, J = 7.6 Hz, 1H), 7.42 (d, J = 5.8 Hz, |
| | | | | | | 1H), 7.33 (t, J = 7.4 Hz, 1H), |
| | | | | | | 6.65 (s, 1H), 6.59 (s, 1H), 5.25- |
| | | | | | | 5.19 (m, 1H), 4.78 (s, 2H), 4.61 |
| | | | | | | (s, 2H), 2.14 (s, 3H), 1.65 (d, J = |
| | | | | | | 6.9 Hz, 3H). LC/MS (ESI) m/z: |
| | | | | | | 507 (M + H)⁺. RT (Method A): |
| | | | | | | 1.49 min. |
| 348^{a} | 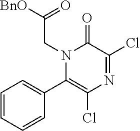 | | | 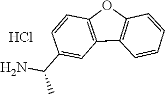 | 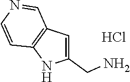 | ¹H NMR (400 MHz, CD₃OD) δ 8.70 (s, 1H), 8.09 (s, 2H), 8.00 (d, J = 7.3 Hz, 1H), 7.57-7.50 (m, 3H), 7.46 (t, J = 7.7 Hz, 1H), 7.39-7.26 (m, 7H), 6.69 (s, 1H), 6.46 (s, 1H), 5.32 (q, J = 6.3 Hz, 1H), 4.52 (s, 4H), 1.69 (d, J = 6.7 Hz, 3H). LC/MS (ESI) m/z: 569 (M + H)⁺. RT (Method A): 1.81 min. |
| 349^{a} | 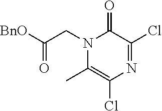 | | | 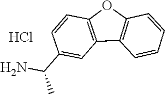 | 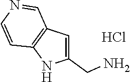 | ¹H NMR (400 MHz, CD₃OD) δ 8.99 (s, 1H), 8.26 (d, J = 6.0 Hz, 1H), 8.04 (s, 1H), 7.98 (d, J = 7.7 Hz, 1H), 7.83 (d, J = 6.2 Hz, 1H), 7.55 (d, J = 8.4 Hz, 1H), 7.51 (s, 2H), 7.45 (d, J = 7.7 Hz, 1H), 7.34 (d, J = 8.1 Hz, 1H), |
| | | | | | | 6.91 (s, 1H), 6.66 (s, 1H), 5.23 |
| | | | | | | (d, J = 6.7 Hz, 1H), 4.80 (s, 2H), |
| | | | | | | 4.68 (s, 2H), 2.15 (s, 3H), 1.65 |
| | | | | | | (d, J = 6.2 Hz, 3H). LC/MS (ESI) |
| | | | | | | m/z: 507 (M + H)⁺. RT (Method |
| | | | | | | A): 1.49 min. |

^{a}Steps 3-5 only.

### Scheme 104. Synthesis of (R)—N-((1H-pyrrolo[3,2-c]pyridine-2-yl)methyl)-2-(5-((1-(dibenzo[b,d]furan-2-yl)ethyl)amino)-2-methoxy-6-oxopyrimidin-1(6H)-yl)acetamide (Compound 319)

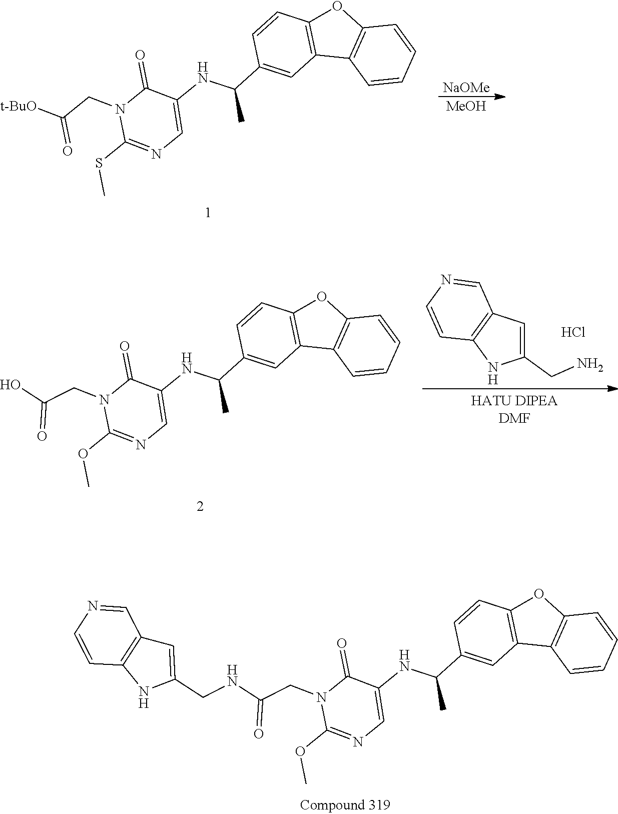

### Step 1: (R)-2-(5-((1-(dibenzo[b,d]furan-2-yl)ethyl)amino)-2-methoxy-6-oxopyrimidin-1(6H)-yl)acetic acid (2)

A solution of tert-butyl (R)-2-(5-((1-(dibenzo[b,d]furan-2-yl)ethyl)amino)-2-(methylthio)-6-oxopyrimidin-1(6H)-yl)acetate (850 mg, 1.82 mmol) in NaOMe/MeOH solution (8 mL, 43 mmol, 30% wt. in MeOH) was stirred at 60° C. for 3 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (710 mg, yield 98.84%) as a yellow solid, which was used directly in the next step without further purification. LC/MS (ESI) m/z: 394 (M+H)⁺.

### Step 2: (R)—N-((1H-pyrrolo[3,2-c]pyridine-2-yl)methyl)-2-(5-((1-(dibenzo[b,d]furan-2-yl)ethyl)amino)-2-methoxy-6-oxopyrimidin-1(6H)-yl)acetamide (Compound 319)

To a solution of (R)-2-(5-((1-(dibenzo[b,d]furan-2-yl)ethyl)amino)-2-methoxy-6-oxopyrimidin-1(6H)-yl)acetic acid (200 mg, 0.51 mmol) in DMF (3 mL) was added (1H-pyrrolo[3,2-c]pyridine-2-yl)methanamine hydrochloride (197 mg, 1.07 mmol), HATU (400 mg, 1.05 mmol) and DIPEA (483 mg, 3.7 mmol) and the reaction mixture was stirred at room temperature for 3 hours. The mixture was diluted with EtOAc, washed with water and brine, dried over anhydrous Na₂SO₄, filtered, and concentrated under reduced pressure to dryness. The residue was purified by flash chromatography (silica gel, 0-20% MeOH in DCM) and further purified by prep-HPLC to give Compound 319 (25 mg, yield 21.7%) as a white solid. ¹H NMR (400 MHz, CD₃OD) δ 8.71 (s, 1H), 8.10 (d, J=5.7 Hz, 1H), 8.03 (s, 1H), 7.98 (d, J=7.7 Hz, 1H), 7.56-7.44 (m, 4H), 7.38 (d, J=5.9 Hz, 1H), 7.33 (t, J=7.3 Hz, 1H), 6.64 (s, 1H), 6.54 (s, 1H), 4.76 (s, 2H), 4.58 (s, 2H), 4.54 (dd, J=6.5 Hz, 1H), 3.76 (s, 3H), 1.61 (d, J=6.6 Hz, 3H). LC/MS (ESI) m/z: 523 (M+H)⁺. RT (Method A): 1.58 min.

### Scheme 105. Synthesis of (R)—N-((1H-pyrrolo[3,2-c]pyridine-2-yl)methyl)-2-(2-(2-fluorophenyl)-6-oxo-5-((1-(2-phenyloxazol-4-yl)ethyl)amino)pyrimidin-1(6H)-yl)acetamide (Compound 321)

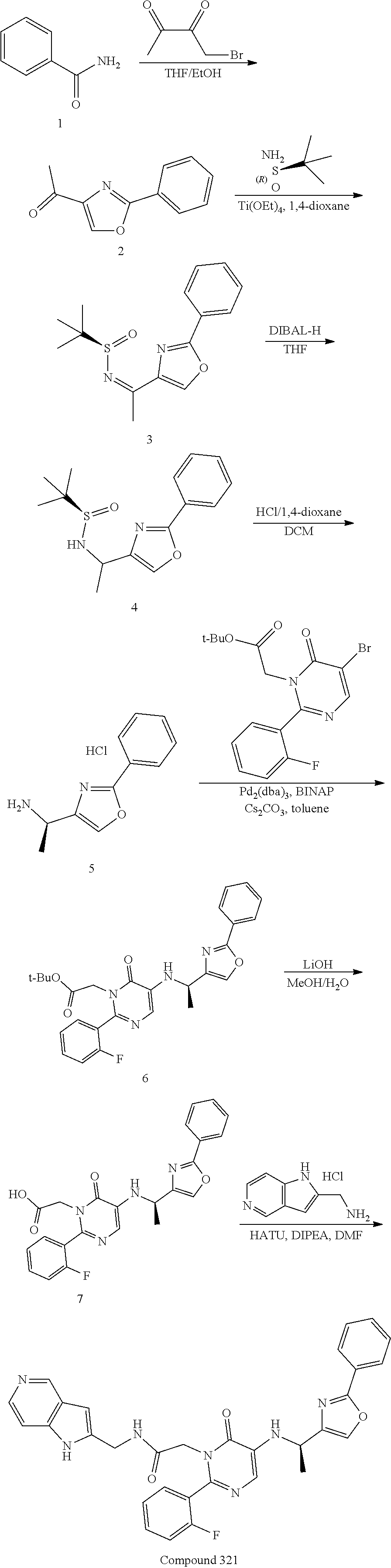

### Step 1: 1-(2-Phenyloxazol-4-yl)ethan-1-one (2)

To a solution of benzamide (2.0 g, 16.51 mmol) in THF/EtOH (20 mL, v/v=1/1) was added 1-bromobutane-2,3-dione (2.7 g, 16.41 mmol) and the reaction mixture was stirred at 80° C. overnight. The mixture was concentrated under reduced pressure and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (600 mg, yield 19.4%) as a yellow solid. LC/MS (ESI) m/z: 209 (M+H)⁺.

### Step 2: (R,Z)-2-methyl-N-(1-(2-phenyloxazol-4-yl)ethylidene)propane-2-sulfinamide (3)

To a mixture of 1-(2-phenyloxazol-4-yl)ethan-1-one (600 mg, 3.20 mmol) and (R)-2-methylpropane-2-sulfinamide (1.55 g, 12.81 mmol) in 1,4-dioxane (10 mL) was added Ti(OEt)₄ (2.92 g, 12.83 mmol) under N₂ atmosphere and the reaction mixture was stirred at 75° C. for 2 hours. The mixture was diluted with EtOAc, washed with water and brine, dried over anhydrous Na₂SO₄, filtered, and concentrated under reduced pressure to give the title compound (800 mg, yield 85.9%) as a brown oil. LC/MS (ESI) m/z: 291 (M+H)⁺.

### Step 3: (R)-2-methyl-N-(I-1-(2-phenyloxazol-4-yl)ethyl)propane-2-sulfinamide (4)

To a solution of ((R,Z)-2-methyl-N-(1-(2-phenyloxazol-4-yl)ethylidene)propane-2-sulfinamide (800 mg, 2.75 mmol) in THF (10 mL) was added DIBAL-H (3.7 mL, 2.75 mmol, 1.5 M in THF) dropwise at −78° C. under N₂ atmosphere and the mixture was stirred under N₂ atmosphere at −78° C. for 10 minutes. The mixture was quenched with saturated aq. Potassium sodium tartrate solution at 0° C. and extracted wit EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (195 mg, yield 24.2%) as a yellow solid. LC/MS (ESI) m/z: 293 (M+H)⁺.

### Step 4: (R)-1-(2-phenyloxazol-4-yl)ethan-1-amine hydrochloride (5)

To a solution of (R)-2-methyl-N—(I-1-(2-phenyloxazol-4-yl)ethyl)propane-2-sulfinamide (190 mg, 0.64 mmol) in DCM (2 mL) was added HCl/1,4-dioxane (1 mL, 4M) and the reaction mixture was stirred under N₂ atmosphere at room temperature for 1 hour. The reaction mixture was concentrated under reduced pressure to dryness to give the title compound (120 mg, yield 98.1%) as a yellow solid, which was used directly in the next step without further purification. LC/MS (ESI) m/z: 189 (M+H)⁺.

### Step 5: Tert-butyl (R)-2-(2-(2-fluorophenyl)-6-oxo-5-((1-(2-phenyloxazol-4-yl)ethyl) amino)pyrimidin-1(6H)-yl)acetate (6)

To a mixture of (R)-1-(2-phenyloxazol-4-yl)ethan-1-amine hydrochloride (120 mg, 0.53 mmol) and tert-butyl 2-(5-bromo-2-(2-fluorophenyl)-6-oxopyrimidin-1(6H)-yl)acetate (300 mg, 0.78 mmol) in toluene (5 mL) was added Cs₂CO₃ (800 mg, 2.45 mmol), BINAP (140 mg, 0.22 mmol), Pd₂(dba)₃ (196 mg, 0.21 mmol) under N₂ atmosphere and the reaction mixture was stirred at 100° C. overnight. The mixture was diluted with EtOAc, washed with brine, dried over anhydrous Na₂SO₄, filtered, and concentrated under reduced pressure to dryness. The residue was purified by flash chromatography (silica gel, 0-30% EtOAc in PE) to give the title compound (210 mg, yield 54.6%) as a yellow solid. LC/MS (ESI) m/z: 491 (M+H)⁺.

### Step 6: (R)-2-(2-(2-fluorophenyl)-6-oxo-5-((1-(2-phenyloxazol-4-yl)ethyl)amino) pyrimidin-1(6H)-yl)acetic acid (7)

To a solution of tert-butyl (R)-2-(2-(2-fluorophenyl)-6-oxo-5-((1-(2-phenyloxazol-4-yl)ethyl)amino)pyrimidin-1(6H)-yl)acetate (200 mg, 0.40 mmol) in MeOH/water (3.0 mL, v/v=4/1) was added LiOH·H₂O (34 mg, 0.78 mmol) and the reaction mixture was stirred at 60° C. overnight. The mixture was acidified with 1 N aq. HCl to pH˜3 and extracted with EtOAc twice. The combined organic layers were dried over anhydrous Na₂SO₄, filtered, and concentrated under reduced pressure to give the title compound (150 mg, yield 84.6%) as a yellow solid, which was used directly in the next step without further purification. LC/MS (ESI) m/z: 435 (M+H)⁺.

### Step 7: (R)—N-((1H-pyrrolo[3,2-c]pyridine-2-yl)methyl)-2-(2-(2-fluorophenyl)-6-oxo-5-((1-(2-phenyloxazol-4-yl)ethyl)amino)pyrimidin-1(6H)-yl)acetamide (Compound 321)

To a mixture of (R)-2-(2-(2-fluorophenyl)-6-oxo-5-((1-(2-phenyloxazol-4-yl)ethyl)amino) pyrimidin-1(6H)-yl)acetic acid (150 mg, 0.34 mmol) and (1H-pyrrolo[3,2-c]pyridine-2-yl)methanamine hydrochloride (75 mg, 0.40 mmol) in DMF (4 mL) was added DIPEA (223 mg, 1.72 mmol) and HATU (157 mg, 0.41 mmol) and the reaction mixture was stirred at room temperature for 1 hour. The mixture was diluted with EtOAc, washed with saturated aq. NaHCO₃ solution, dried over anhydrous Na₂SO₄, filtered, and concentrated under reduced pressure to dryness. The residue was purified by flash chromatography (silica gel, 0-20% MeOH in DCM) and further purified by prep-HPLC to give Compound 321 (25.3 mg, yield 13.0%) as a white solid. ¹H NMR (400 MHz, DMSO-d₆) δ 11.28 (s, 1H), 8.73 (s, 1H), 8.59 (s, 1H), 8.11 (d, J=7.6 Hz, 2H), 7.99-7.96 (m, 2H), 7.53 (s, 4H), 7.40 (t, J=7.1 Hz, 1H), 7.33 (d, J=9.6 Hz, 1H), 7.30 (s, 2H), 7.19 (t, J=7.4 Hz, 1H), 6.22 (s, 1H), 5.55 (d, J=7.3 Hz, 1H), 4.92-4.58 (m, 2H), 4.37-4.12 (m, 3H), 1.58 (d, J=6.2 Hz, 3H). LC/MS (ESI) m/z: 564 (M+H)⁺. RT (Method A): 1.35 min.

Compound 347 was prepared based on Scheme 105:

| | Reactant | Reactant | Reactant | Reactant | Reactant | Characterization |
|---|---|---|---|---|---|---|
| # | A | B | C | D | E | Data |
| 347 | 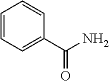 | 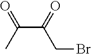 | 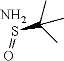 | 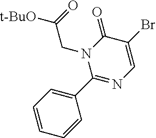 | 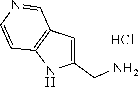 | ¹H NMR (400 MHz, CD₃OD) δ 8.70 (s, 1H), 8.09 (d, J = 5.3 Hz, 1H), 8.01 (d, J = 2.3 Hz, 2H), 7.89 (s, 1H), 7.51-7.43 (m, 6H), 7.37 (t, J = 7.0 Hz, 3H), 7.21 (s, 1H), 6.47 (s, 1H), 4.65 (m, 1H), 4.62 (s, 2H), 4.53 (s, 2H), 1.67 (d, J = 6.3 Hz, 3H). LC/MS (ESI) m/z: 546 (M + H)⁺. RT (Method A): 1.31 min. |

### Scheme 106. Synthesis of (R)-2-(5-((1-(dibenzo[b,d]furan-2-yl)ethyl)amino)-2-(2-fluorophenyl)-6-oxopyrimidin-1(6H)-yl)-N-((3-fluoro-1H-pyrrolo[3,2-c]pyridine-2-yl)methyl) acetamide (Compound 331)

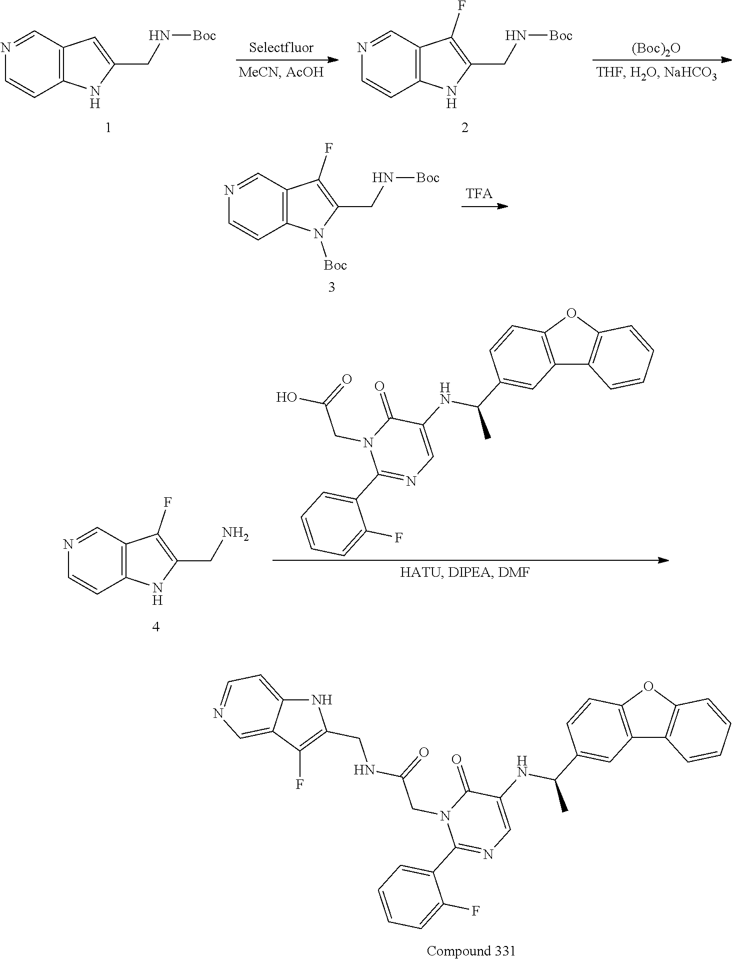

### Step 1: Tert-butyl ((3-fluoro-1H-pyrrolo[3,2-c]pyridine-2-yl)methyl)carbamate (2)

To a mixture of tert-butyl ((1H-pyrrolo[3,2-c]pyridine-2-yl)methyl)carbamate (380 mg, 1.54 mmol) in Acetonitrile (5 mL)/Acetic Acid (1 mL) was added Selectfluor (981 mg, 2.77 mmol) and the reaction mixture was stirred at 50° C. for 2 hours. The mixture was diluted with EtOAc, washed with saturated aq. NaHCO₃ solution and brine, dried over anhydrous Na₂SO₄, filtered, and concentrated under reduced pressure to dryness to give the title compound (200 mg, yield 48.9%) as a yellow solid, which was used in next step without purification. LC/MS (ESI) m/z: 266 (M+H)⁺.

### Step 2: Tert-butyl 2-(((tert-butoxycarbonyl)amino)methyl)-3-fluoro-1H-pyrrolo[3,2-c]pyridine-1-carboxylate (3)

To a mixture of tert-butyl ((3-fluoro-1H-pyrrolo[3,2-c]pyridine-2-yl)methyl)carbamate (200 mg, 0.81 mmol) and Di-tert-butyl dicarbonate (72 mg, 0.39 mmol) in THF (2 mL) was added saturated aq. NaHCO₃ solution (2 mL) and the reaction mixture was stirred at room temperature for 2 hour. The mixture was diluted with EtOAc, washed with brine, dried over anhydrous Na₂SO₄, filtered, and concentrated under reduced pressure to dryness. The residue was purified by flash chromatography (silica gel, 0-10% MeOH in DCM) and further purified by prep-HPLC to give the title compound (70 mg, yield 25%) as a yellow solid. LC/MS (ESI) m/z: 366 (M+H)⁺.

### Step 3: (3-Fluoro-1H-pyrrolo[3,2-c]pyridine-2-yl)methanamine (4)

A solution of tert-butyl tert-butyl 2-(((tert-butoxycarbonyl)amino)methyl)-3-fluoro-1H-pyrrolo[3,2-c]pyridine-1-carboxylate (70 mg, 0.19 mmol) in TFA (1 mL) was stirred under N₂ atmosphere at room temperature for 1 hour. The reaction mixture was concentrated to dryness under reduced pressure to give the title compound (110 mg, 100% yield) as a yellow oil, which was used directly in the next step without further purification. LC/MS (ESI) m/z: 166 (M+H)⁺.

### Step 4: (R)2-(5-((1-(dibenzo[b,d]furan-2-yl)ethyl)amino)-2-(2-fluorophenyl)-6-oxopyrimidin-1(6H)-yl)-N-((3-fluoro-1H-pyrrolo[3,2-c]pyridine-2-yl)methyl) acetamide (Compound 331)

To a mixture of I-2-(5-((1-(dibenzo[b,d]furan-2-yl)ethyl)amino)-2-(2-fluorophenyl)-6-oxopyrimidin-1(6H)-yl)acetic acid (50 mg, 0.11 mmol) and (3-fluoro-1H-pyrrolo[3,2-c]pyridine-2-yl)methanamine (21 mg, 0.13 mmol) in DMF (3 mL) was added DIPEA (70 mg, 0.55 mmol) and HATU (50 mg, 0.13 mmol) at 0° C. under N₂ atmosphere and the mixture was stirred at room temperature for 1 hour. The mixture was quenched with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=8:1) and further purified by prep-HPLC to give Compound 331 (20 mg, yield 30.0%) as a white solid. ¹H NMR (400 MHz, CD₃OD) δ 8.74 (s, 1H), 8.16 (d, J=6.3 Hz, 1H), 8.08 (s, 1H), 8.02 (d, J=7.4 Hz, 1H), 7.56 (d, J=9.1 Hz, 3H), 7.47 (t, J=7.3 Hz, 1H), 7.40-7.26 (m, 4H), 7.12 (s, 1H), 6.98 (s, 1H), 6.92 (s, 1H), 5.05-4.93 (m, 1H), 4.68 (d, J=6.5 Hz, 1H), 4.44 (s, 2H), 4.27 (s, 1H), 1.67 (d, J=6.5 Hz, 3H). LC/MS (ESI) (m/z): 605 (M+H)⁺. RT (Method A): 1.74 min.

### Scheme 107. Synthesis of ((R)—N-((1H-pyrrolo[3,2-c]pyridine-2-yl)methyl)-2-(5-((1-(dibenzo[b,d]furan-2-yl)-2-methylpropyl)amino)-2-(2-fluorophenyl)-6-oxopyrimidin-1(6H)-yl)acetamide (Compound 332)

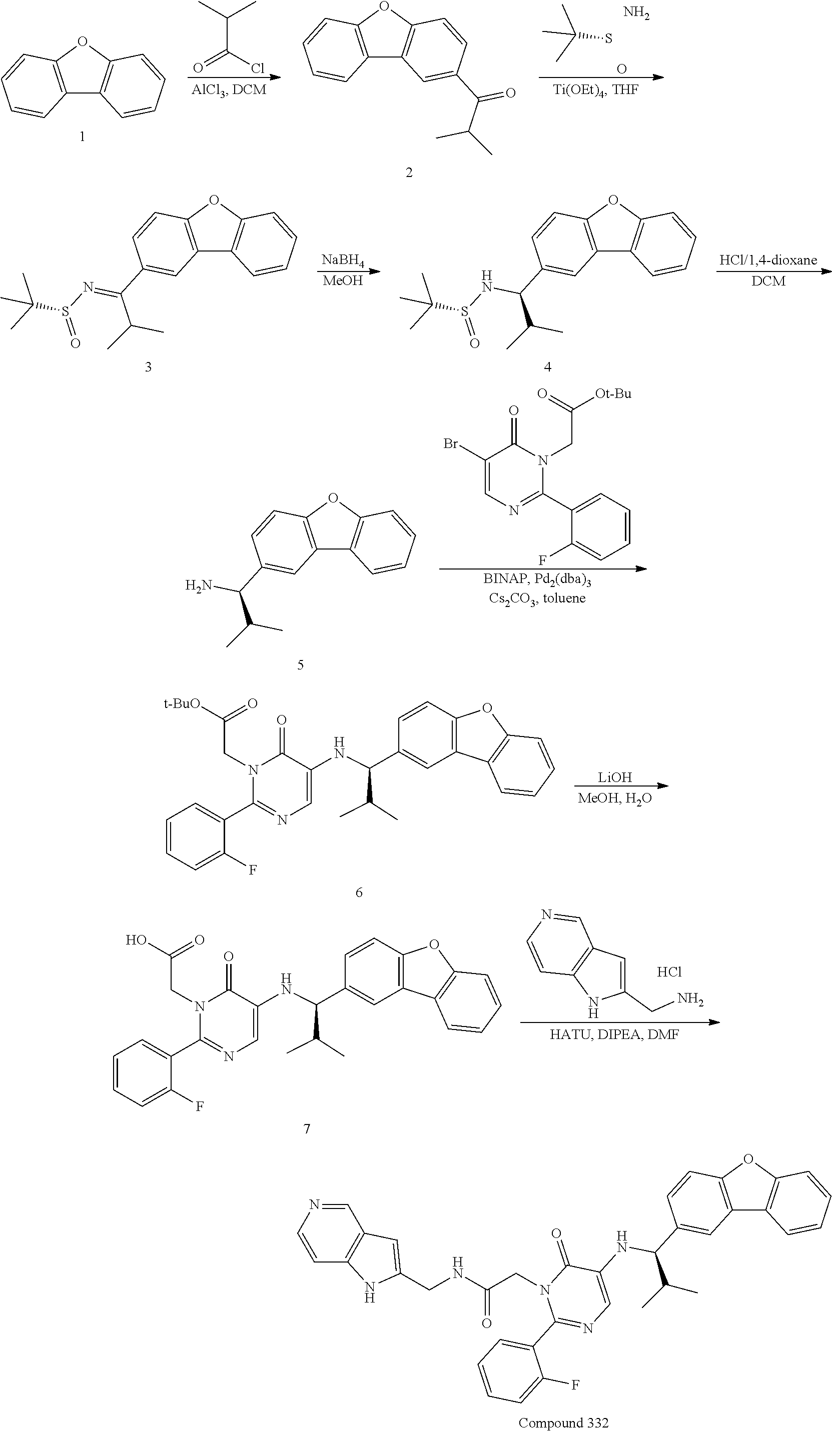

### Step 1: 1-(Dibenzo[b,d]furan-2-yl)-2-methylpropan-1-one (2)

To a mixture of dibenzo[b,d]furan (5.00 g, 29.7 mmol) and isobutyryl chloride (6.36 g, 59.4 mmol) in DCM (30 mL) was added AlCl₃ (8.00 g, 60.1 mmol) at 0° C. and the reaction mixture was stirred at room temperature overnight. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (6.30 g, yield 88.9%) as a yellow oil. LC/MS (ESI) m/z: 239 (M+H)⁺.

### Step 2: (R,Z)—N-(1-(dibenzo[b,d]furan-2-yl)-2-methylpropylidene)-2-methylpropane-2-sulfinamide (3)

To a mixture of 1-(dibenzo[b,d]furan-2-yl)-2-methylpropan-1-one (2.00 g, 8.40 mmol) and (R)-2-methylpropane-2-sulfinamide (3.10 g, 25.6 mmol) in THF (10 mL) was added titanium ethoxide (5.80 g, 25.4 mmol) and the mixture was stirred at 70° C. for 12 hours. The mixture was quenched with water and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give the title compound (2.00 g, yield 71.4%) as a yellow solid. LC/MS (ESI) m/z: 342 (M+H)⁺.

### Step 3: (R)—N—((R)-1-(dibenzo[b,d]furan-2-yl)-2-methylpropyl)-2-methylpropane-2-sulfinamide (4)

To a solution of (R,Z)—N-(1-(dibenzo[b,d]furan-2-yl)-2-methylpropylidene)-2-methylpropane-2-sulfinamide (1.50 g, 4.39 mmol) in MeOH (15 mL) was added NaBH₄ (0.16 g, 4.39 mmol) at 0° C. and the reaction mixture was stirred at 25° C. for 1 hour. The mixture was quenched with 1N aq. HCl solution and extracted with EtOAc twice. The organic layers were washed with water and brine, dried over anhydrous Na₂SO₄, filtered, and concentrated under reduced pressure to dryness. The residue was purified by flash chromatography (silica gel, 0-20% EtOAc in PE) to give the title compound (647 mg, yield 43.1%) as a white solid. LC/MS (ESI) m/z: 344 (M+H)⁺.

### Step 4: (R)-1-(dibenzo[b,d]furan-2-yl)-2-methylpropan-1-amine (5)

To a solution of (R)—N—(I-1-(dibenzo[b,d]furan-2-yl)-2-methylpropyl)-2-methyl-propane-2-sulfinamide (647 mg, 1.88 mmol) in DCM (2 mL) was added HCl/1,4-dioxane (4 mL, 4M) and the mixture was stirred under N₂ atmosphere at room temperature for 2 hours. The reaction mixture was concentrated under reduced pressure to dryness. The residue was neutralized with saturated aq. NaHCO₃ solution and extracted with DCM twice. The combined organic layers were washed with brine, dried over anhydrous Na₂SO₄, filtered, and concentrated to dryness to give the title compound (150 mg, yield 33.3%) as a white solid, which was used directly in the next step without further purification. LC/MS (ESI) m/z: 240 (M+H)⁺.

### Step 5: Tert-butyl (R)-2-(5-((1-(dibenzo[b,d]furan-2-yl)-2-methylpropyl)amino)-2-(2-fluorophenyl)-6-oxopyrimidin-1(6H)-yl)acetate (6)

To a mixture of (R)-1-(dibenzo[b,d]furan-2-yl)-2-methylpropan-1-amine (150 mg, 0.62 mmol) and tert-butyl 2-(5-bromo-2-(2-fluorophenyl)-6-oxopyrimidin-1(6H)-yl)acetate (160 mg, 0.41 mmol) in toluene (3 mL) was added Cs₂CO₃ (410 mg, 1.25 mmol), BINAP (26.0 mg, 0.04 mmol) and Pd₂(dba)₃ (38.0 mg, 0.04 mmol). The reaction mixture was degassed under N₂ atmosphere for three times and stirred at 110° C. overnight. The mixture was diluted with water and extracted with EtOAc twice. The combined organic layers were washed with water and brine, dried over anhydrous Na₂SO₄, filtered, and concentrated under reduced pressure to dryness. The residue was purified by flash chromatography (silica gel, 0-20% EtOAc in PE) to give the title compound (106 mg, yield 31.2%) as a yellow oil. LC/MS (ESI) m/z: 542 (M+H)⁺.

### Step 6: (R)-2-(5-((1-(dibenzo[b,d]furan-2-yl)-2-methylpropyl)amino)-2-(2-fluorophenyl)-6-oxopyrimidin-1(6H)-yl)acetic acid (7)

To a solution of tert-butyl (R)-2-(5-((1-(dibenzo[b,d]furan-2-yl)-2-methylpropyl) amino)-2-(2-fluorophenyl)-6-oxopyrimidin-1(6H)-yl)acetate (105 mg, 0.19 mmol) in MeOH (3 mL) and water (1 mL) was added LiOH (25 mg, 0.48 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (94 mg, yield 99.9%) as a white solid, which was used directly in the next step without further purification. LC/MS (ESI) m/z: 486 (M+H)⁺.

### Step 7: (R)—N-((1H-pyrrolo[3,2-c]pyridine-2-yl)methyl)-2-(5-((1-(dibenzo[b,d]furan-2-yl)-2-methylpropyl)amino)-2-(2-fluorophenyl)-6-oxopyrimidin-1(6H)-yl)acetamide (Compound 332)

To a mixture of 2-(5-(([1,1′-Biphenyl]-4-ylmethyl)amino)-6-oxo-2-(4-(pentafluoro-16-sulfaneyl)phenyl)pyrimidin-1(6H)-yl)acetic acid (132 mg, 0.27 mmol) and (1H-pyrrolo[3,2-c]pyridine-2-yl)methanamine hydrochloride (75 mg, 0.40 mmol) in DMF (3 mL) was added HATU (124 mg, 0.32 mmol), DIPEA (105 mg, 0.81 mmol) and the mixture was stirred at room temperature for 0.5 hour. The mixture was diluted with water and extracted with EtOAc twice. The combined organic layers were washed with brine, dried over anhydrous Na₂SO₄, filtered, and concentrated under reduced pressure to dryness. The residue was purified flash chromatography (silica gel, 0-12% MeOH in DCM) and further purified by prep-HPLC to give Compound 332 (5.50 mg, yield 3.29%) as a white solid. ¹H NMR (400 MHz, CD₃OD) δ 8.73 (s, 1H), 8.13 (s, 1H), 8.05 (s, 1H), 8.00 (d, J=7.3 Hz, 1H), 7.56 (d, J=8.7 Hz, 2H), 7.51-7.39 (m, 4H), 7.34 (t, J=7.4 Hz, 2H), 7.15 (t, J=8.3 Hz, 1H), 7.07 (s, 1H), 6.97 (s, 1H), 6.45 (s, 1H), 4.46 (s, 2H), 4.27 (d, J=6.6 Hz, 2H), 2.27-2.19 (m, 1H), 1.29 (s, 1H), 1.16 (d, J=6.0 Hz, 3H), 0.94 (d, J=6.3 Hz, 3H). LC/MS (ESI) m/z: 615 (M+H)⁺. RT (Method A): 1.95 min.

### Scheme 108. Synthesis of (R)—N-((1H-pyrrolo[3,2-c]pyridine-2-yl)methyl)-2-(3-((1-(dibenzo[b,d]furan-2-yl)ethyl)amino)-5-methyl-2-oxopyrazin-1(2H)-yl)acetamide (Compound 333)

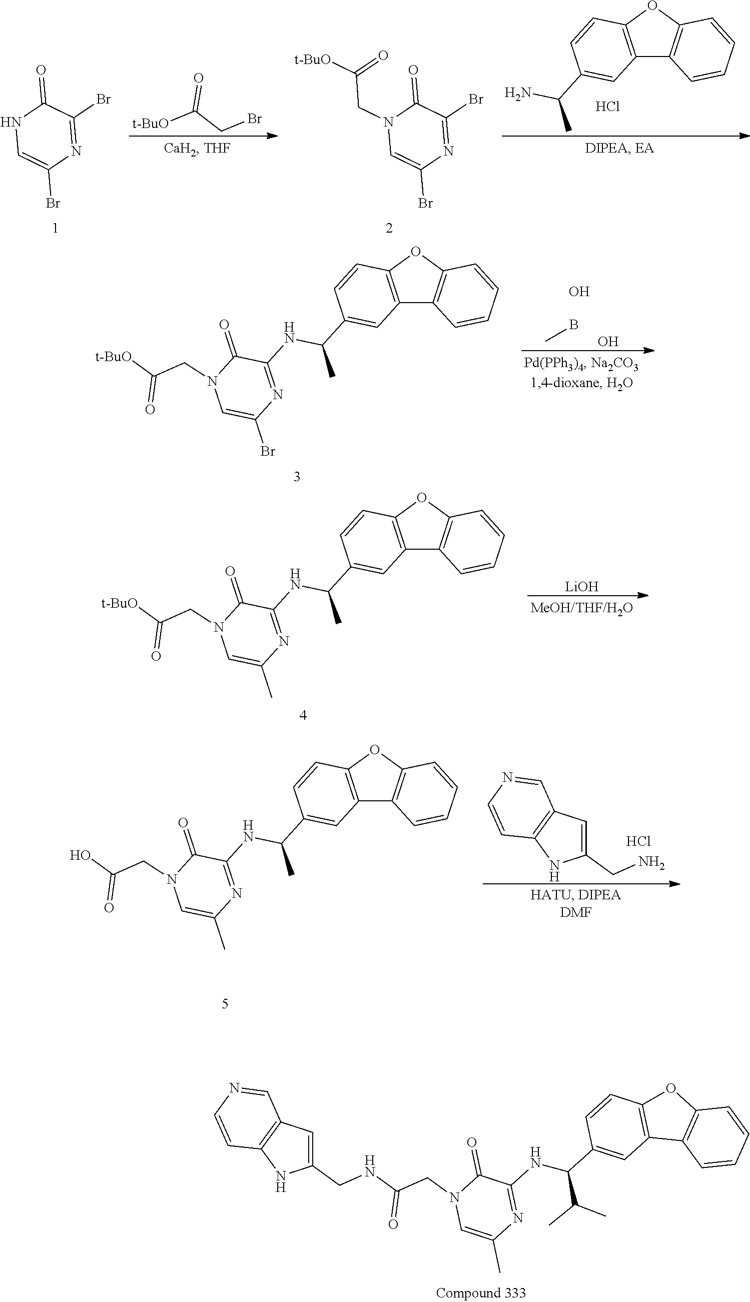

### Step 1: Tert-butyl 2-(3,5-dibromo-2-oxopyrazin-1(2H)-yl)acetate (2)

To a mixture of 3,5-dibromopyrazin-2(1H)-one (900 mg, 0.82 mmol) and tert-butyl 2-bromoacetate (2.1 g, 10.63 mmol) in THF (10 mL) was added CaH₂ (447 mg, 10.63 mmol) at 0° C. under N₂ atmosphere and the mixture was stirred at 70° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give the title compound (625 mg, yield 48.2%) as a white solid. LC/MS (ESI) m/z: 367 (M+H)⁺.

### Step 2: Tert-butyl (R)-2-(5-bromo-3-((1-(dibenzo[b,d]furan-2-yl)ethyl)amino)-2-oxopyrazin-1(2H)-yl)acetate (3)

To a mixture of tert-butyl 2-(3,5-dibromo-2-oxopyrazin-1(2H)-yl)acetate (300 mg, 0.82 mmol) and I-1-(dibenzo[b,d]furan-2-yl)ethan-1-amine hydrochloride (173 mg, 0.82 mmol) in EtOAc (5 mL) was added DIPEA (318 mg, 2.46 mmol) at room temperature. Then the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give the title compound (245 mg, yield 60.0%) as a white solid. LC/MS (ESI) m/z: 498 (M+H)⁺.

### Step 3: Tert-butyl (R)-2-(3-((1-(dibenzo[b,d]furan-2-yl)ethyl)amino)-5-methyl-2-oxopyrazin-1(2H)-yl)acetate (4)

To a mixture of tert-butyl (R)-2-(5-bromo-3-((1-(dibenzo[b,d]furan-2-yl)ethyl)amino)-2-oxopyrazin-1(2H)-yl)acetate (240 mg, 0.48 mmol) and methylboronic acid (87 mg, 1.45 mmol) in 1,4-dioxane (8 mL) and water (2 mL) was added Na₂CO₃ (153 mg, 1.45 mmol) and Pd(PPh₃)₄ (56 mg, 0.05 mmol) at room temperature. Then the reaction mixture was degassed under N₂ atmosphere for three times and stirred at 90° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give the title compound (46 mg, yield 22.1%) as a white solid. LC/MS (ESI) m/z: 434 (M+H)⁺.

### Step 4: (R)-2-(3-((1-(dibenzo[b,d]furan-2-yl)ethyl)amino)-5-methyl-2-oxopyrazin-1(2H)-yl)acetic acid (5)

To a solution of tert-butyl (R)-2-(3-((1-(dibenzo[b,d]furan-2-yl)ethyl)amino)-5-methyl-2-oxopyrazin-1(2H)-yl)acetate (45 mg, 0.11 mmol) in MeOH/THF/water (5 mL, v/v/v=2/2/1) was added LiOH·H₂O (13 mg, 0.31 mmol) and the mixture was stirred at room temperature overnight. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (30 mg, yield 76.6%) as a yellow solid, which was used directly in the next step 242yridin further purification. LC/MS (ESI) m/z: 378 (M+H)⁺.

### Step 5: (R)—N-((1H-pyrrolo[3,2-c]pyridine-2-yl)methyl)-2-(3-((1-(dibenzo[b,d]furan-2-yl)ethyl)amino)-5-methyl-2-oxopyrazin-1(2H)-yl)acetamide (Compound 333)

To a mixture of (R)-2-(3-((1-(dibenzo[b,d]furan-2-yl)ethyl)amino)-5-methyl-2-oxopyrazin-1(2H)-yl)acetic acid (30 mg, 0.08 mmol) and (1H-pyrrolo[3,2-c]pyridine-2-yl)methanamine hydrochloride (17 mg, 0.12 mmol) in DMF (3 mL) was added DIPEA (41 mg, 0.32 mmol) and HATU (39 mg, 0.11 mmol) and the reaction mixture was stirred at room temperature for 1 hour. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-10% MeOH in DCM) and further purified by prep-HPLC to give Compound 333 (4.3 mg, yield 10.7%) as a white solid. ¹H NMR (400 MHz, DMSO-d₆) δ 11.35 (s, 1H), 8.73 (s, 2H), 8.18 (s, 1H), 8.11 (d, J=6.4 Hz, 2H), 7.68 (d, J=8.4 Hz, 1H), 7.64-7.58 (m, 2H), 7.51 (t, J=7.6 Hz, 1H), 7.38 (d, J=7.7 Hz, 2H), 7.31 (d, J=5.4 Hz, 1H), 6.55 (s, 1H), 6.42 (s, 1H), 5.31 (s, 1H), 4.51-4.42 (m, 4H), 1.97 (s, 3H), 1.59 (d, J=6.7 Hz, 3H). LC/MS (ESI) m/z: 507 (M+H)⁺. RT (Method A): 1.61 min.

### Scheme 109. Synthesis of (R)—N-((1H-pyrrolo[3,2-c]pyridine-2-yl)methyl)-2-(5-((1-(dibenzo[b,d]furan-2-yl)ethyl)amino)-2-isopropyl-6-oxopyrimidin-1(6H)-yl)acetamide (Compound 341)

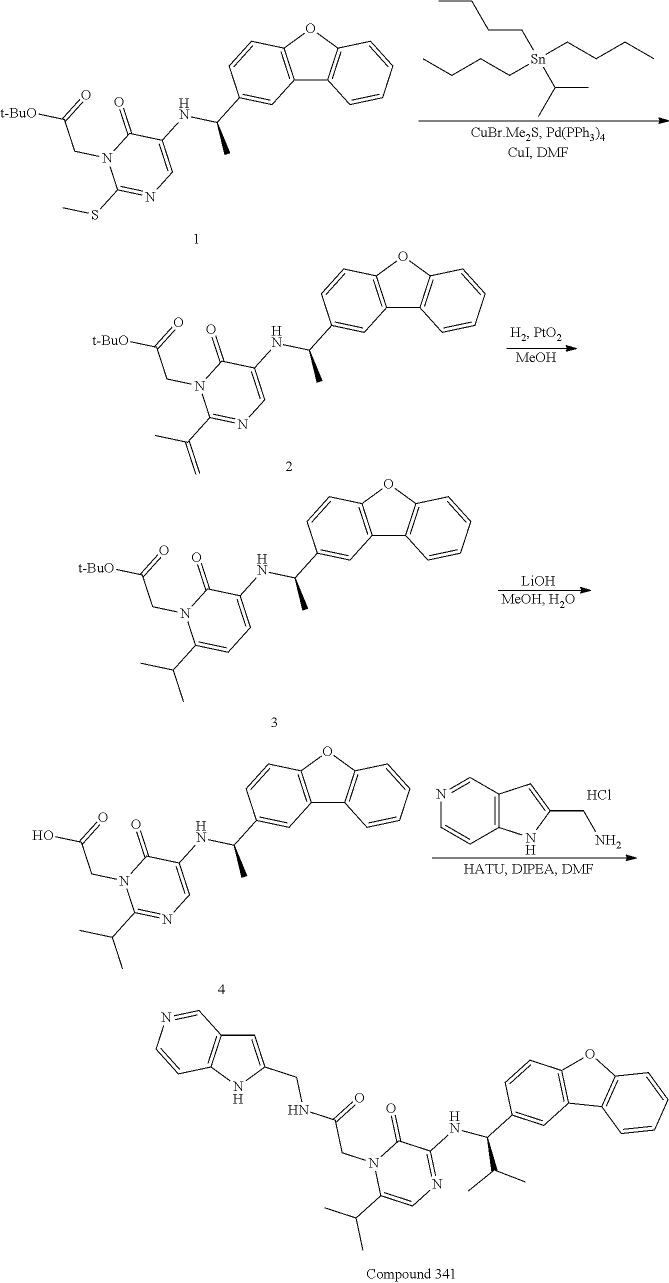

### Step 1: Tert-butyl (R)-2-(5-((1-(dibenzo[b,d]furan-2-yl)ethyl)amino)-6-oxo-2-(prop-1-en-2-yl)pyrimidin-1(6H)-yl)acetate (2)

To a mixture of tert-butyl (R)-2-(5-((1-(dibenzo[b,d]furan-2-yl)ethyl)amino)-2-(methylthio)-6-oxopyrimidin-1(6H)-yl)acetate (500 mg, 1.07 mmol) and tributyl(prop-1-en-2-yl)stannane (535 mg, 1.61 mmol) in DMF (6 mL) was added CuI (41 mg, 0.21 mmol), Copper(I) bromide-dimethyl sulfide complex (332 mg 1.61 mmol) and Pd(PPh₃)₄ (248 mg, 0.21 mmol) under N₂ atmosphere, the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give the title compound (150 mg, yield 30.4%) as a yellow oil. LC/MS (ESI) m/z: 460 (M+H)⁺.

### Step 2: Tert-butyl (R)-2-(5-((1-(dibenzo[b,d]furan-2-yl)ethyl)amino)-2-isopropyl-6-oxopyrimidin-1(6H)-yl)acetate (3)

To a solution of tert-butyl (R)-2-(5-((1-(dibenzo[b,d]furan-2-yl)ethyl)amino)-6-oxo-2-(prop-1-en-2-yl)pyrimidin-1(6H)-yl)acetate (150 mg, 0.32 mmol) in MeOH (3 mL) was added PtO₂ (7 mg, 0.03 mmol), the mixture was degassed under N₂ atmosphere for three times and stirred under a H₂ balloon at 35° C. overnight. The mixture was filtered, and the filtrate was concentrated to dryness to give the title compound (150 mg, yield 99.9%) as a yellow solid, which was used directly in the next step without further purification. LC/MS (ESI) m/z: 462 (M+H)⁺.

### Step 3: (R)-2-(5-((1-(dibenzo[b,d]furan-2-yl)ethyl)amino)-2-isopropyl-6-oxopyrimidin-1(6H)-yl)acetic acid (5)

To a solution of tert-butyl (R)-2-(5-((1-(dibenzo[b,d]furan-2-yl)ethyl)amino)-2-isopropyl-6-oxopyrimidin-1(6H)-yl)acetate (156 mg, 0.33 mmol) in MeOH (3 mL) and water (1 mL) was added LiOH (43 mg, 1.02 mmol) and the mixture was stirred at 45° C. for 6 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (120 mg, yield 87.5%) as a colorless oil, which was used directly in the next step without further purification. LC/MS (ESI) m/z: 406 (M+H)⁺.

### Step 4: (R)—N-((1H-pyrrolo[3,2-c]pyridine-2-yl)methyl)-2-(5-((1-(dibenzo[b,d]furan-2-yl)ethyl)amino)-2-isopropyl-6-oxopyrimidin-1(6H)-yl)acetamide (Compound 341)

To a mixture of (R)-2-(5-((1-(dibenzo[b,d]furan-2-yl)ethyl)amino)-2-isopropyl-6-oxopyrimidin-1(6H)-yl)acetic acid (190 mg, 0.46 mmol) and (1H-pyrrolo[3,2-c]pyridine-2-yl)methanamine hydrochloride (103 mg, 0.70 mmol) in DMF (4 mL) was added DIPEA (181 mg, 1.40 mmol) and HATU (220 mg, 0.31 mmol) at room temperature and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 341 (20.5 mg, yield 10.4%) as a white solid. ¹H NMR (400 MHz, CD₃OD) δ 8.71 (s, 1H), 8.10 (d, J=5.2 Hz, 1H), 8.04 (s, 1H), 7.97 (d, J=7.6 Hz, 1H), 7.58-7.49 (m, 3H), 7.46 (t, J=8.3 Hz, 1H), 7.38 (d, J=5.1 Hz, 1H), 7.33 (t, J=7.0 Hz, 1H), 6.85 (s, 1H), 6.57 (s, 1H), 4.92 (s, 2H), 4.63-4.56 (m, 3H), 2.97-2.87 (m, 1H), 1.63 (d, J=6.3 Hz, 3H), 1.15 (d, J=6.3 Hz, 3H), 1.09 (d, J=6.3 Hz, 3H). LC/MS (ESI) m/z: 535 (M+H)⁺. RT (Method A): 1.67 min.

Compound 442 was prepared based on Scheme 109:

| # | Reactant A | Reactant B | Reactant C | Characterization Data |
|---|---|---|---|---|
| 442 | 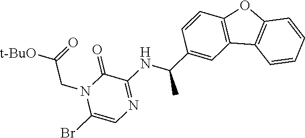 | 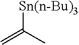 | 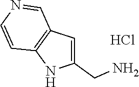 [SMILES: NCc1cc2cnccc2[nH]1 ; Cl] | ¹H NMR (400 MHz, DMSO-d₆) δ 11.34 (s, 1H), 8.78 (t, J = 5.6 Hz, 1H), 8.73 (s, 1H), 8.15 (s, 1H), 8.11 (d, J = 5.8 Hz, 2H), 7.67 (d, J = 8.2 Hz, 1H), 7.63-7.55 (m, 2H), 7.50 (t, J = 7.7 Hz, 1H), 7.39 (t, J = 7.5 Hz, 1H), 7.32 (d, J = 5.6 Hz, 1H), 7.25 (d, J= 8.1 Hz, 1H), 6.65 (s, 1H), 6.42 (s, 1H), 5.27- 5.12 (m, 1H), 4.79-4.59 (m, 2H), 4.46 (d, J = 5.5 Hz, 2H), 2.74- 2.62 (m, 1H), 1.58 (d, J = 7.0 Hz, 3H), 1.12 (d, J = 6.7 Hz, 3H), 1.08 (d, J = 6.7 Hz, 3H). LC/MS (ESI) (m/z): 535 (M + H)⁺. RT (method A): 1.72 min. |

### Scheme 110. Synthesis of (R)—N-((1H-pyrrolo[3,2-c]pyridine-2-yl)methyl)-2-(2-cyclopropoxy-5-((1-(dibenzo[b,d]furan-2-yl)ethyl)amino)-6-oxopyrimidin-1(6H)-yl)acetamide (Compound 343)

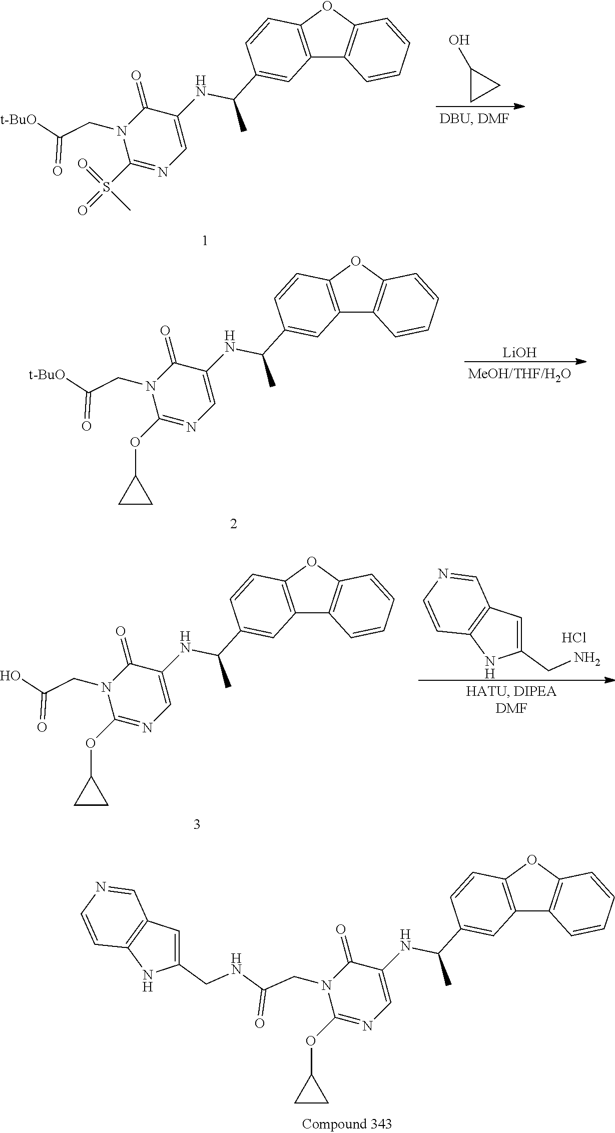

### Step 1: Tert-butyl (R)-2-(2-cyclopropoxy-5-((1-(dibenzo[b,d]furan-2-yl)ethyl)amino)-6-oxopyrimidin-1(6H)-yl)acetate (2)

To a mixture of tert-butyl (R)-2-(5-((1-(dibenzo[b,d]furan-2-yl)ethyl)amino)-2-(methylsulfonyl)-6-oxopyrimidin-1(6H)-yl)acetate (177 mg, 0.35 mmol) and cyclopropanol (93 mg, 2.1 mmol) in DMF (3 mL) was added DBU (150 mg, 1.0 mmol) and the mixture was stirred at 80° C. overnight. The mixture was concentrated under reduced pressure to dryness. The residue was purified by flash chromatography (silica gel, 0-10% EtOAc in PE) to give the title compound (141 mg, yield 83.3%) as a yellow solid. LC/MS (ESI) m/z: 476 (M+H)⁺.

### Step 2: (R)-2-(2-cyclopropoxy-5-((1-(dibenzo[b,d]furan-2-yl)ethyl)amino)-6-oxopyrimidin-1(6H)-yl)acetic acid (3)

To a solution of tert-butyl (R)-2-(2-cyclopropoxy-5-((1-(dibenzo[b,d]furan-2-yl)ethyl)amino)-6-oxopyrimidin-1(6H)-yl)acetate (141 mg, 0.29 mmol) in MeOH (5 mL) was added LiOH (15 mg, 0.62 mmol) at 50° C. for 16 hours. The mixture was acidified with 1N aq. HCl to pH˜5 and extracted with EtOAc twice. The combined organic layers were washed with brine, dried over anhydrous Na₂SO₄, filtered, and concentrated to dryness to give the title compound (124 mg, yield 99.7%) as a yellow solid, which was used directly in the next step without further purification. LC/MS (ESI) m/z: 420 (M+H)⁺.

### Step 3: (R)—N-((1H-pyrrolo[3,2-c]pyridine-2-yl)methyl)-2-(2-cyclopropoxy-5-((1-(dibenzo[b,d]furan-2-yl)ethyl)amino)-6-oxopyrimidin-1(6H)-yl)acetamide (Compound 343)

To a mixture of (R)-2-(2-cyclopropoxy-5-((1-(dibenzo[b,d]furan-2-yl)ethyl)amino)-6-oxopyrimidin-1(6H)-yl)acetic acid (157 mg, 0.37 mmol) and (1H-pyrrolo[3,2-c]pyridine-2-yl)methanamine hydrochloride (101 mg, 0.55 mmol) in DMF (3 mL) was added HATU (209 mg, 0.55 mmol) and DIPEA (238 mg, 1.85 mmol) under N₂ atmosphere and the reaction mixture was stirred at room temperature for 1 hour. The mixture was concentrated under reduced pressure to dryness. The residue was purified by flash chromatography (silica gel, 0-15% MeOH in DCM) and further purified by prep-HPLC to give Compound 343 (25.0 mg, yield 12.3%) as a white solid. ¹H NMR (400 MHz, CD₃OD) δ 8.70 (M, 1H), 8.10 (d, J=5.8 Hz, 1H), 8.04 (d, J=1.0 Hz, 1H), 7.99 (d, J=7.5 Hz, 1H), 7.57-7.51 (m, 3H), 7.49-7.44 (m, 1H), 7.38 (d, J=5.8 Hz, 1H), 7.34 (t, J=7.2 Hz, 1H), 6.63 (s, 1H), 6.53 (s, 1H), 4.70 (s, 2H), 4.57-4.53 (m, 3H), 4.04 (m, 2.9 Hz, 1H), 1.62 (d, J=6.7 Hz, 3H), 0.60-0.52 (m, 4H). LC/MS (ESI) m/z: 549 (M+H)⁺. RT (Method A): 1.66 min.

### Scheme 111. Synthesis of (R)—N-((1H-pyrrolo[3,2-c]pyridine-2-yl)methyl)-2-(5-((1-(dibenzo[b,d]furan-2-yl)ethyl)amino)-6-oxo-2-(1H-pyrazol-4-yl)pyrimidin-1(6H)-yl)acetamide (Compound 360)

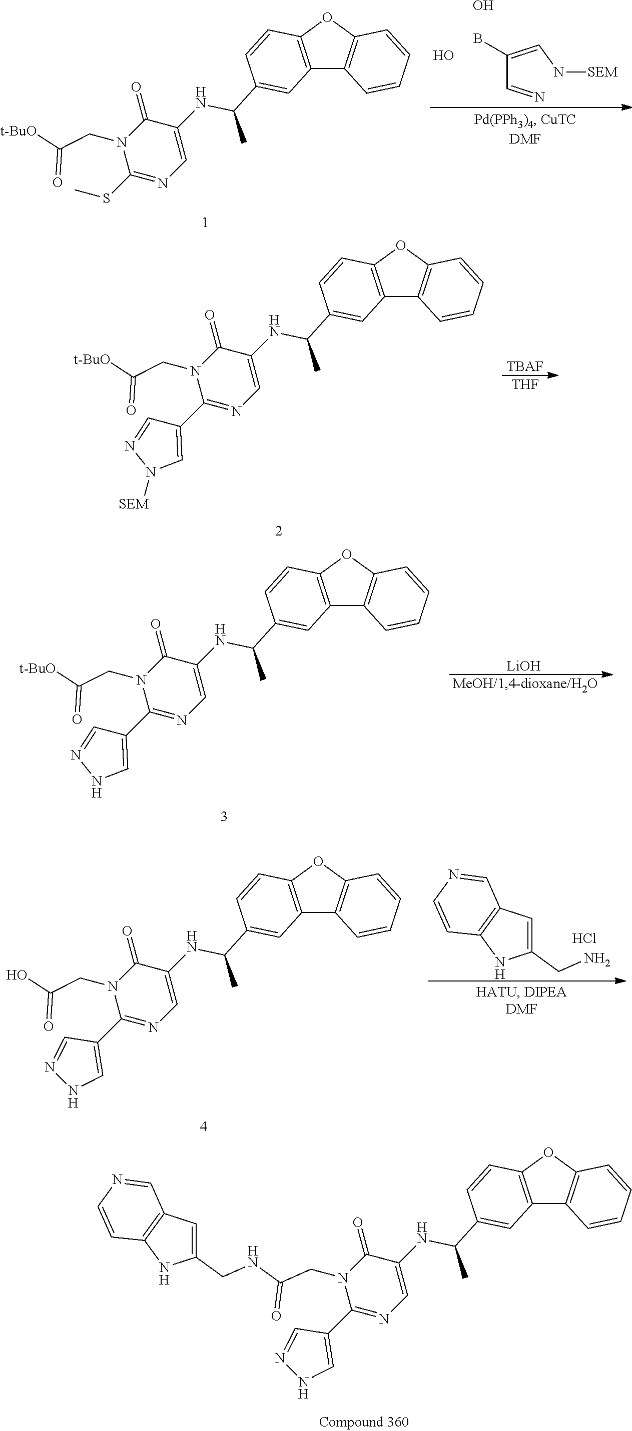

### Step 1: Tert-butyl (R)-2-(5-((1-(dibenzo[b,d]furan-2-yl)ethyl)amino)-6-oxo-2-(1-((2-(trimethylsilyl)ethoxy)methyl)-1H-pyrazol-4-yl)pyrimidin-1(6H)-yl)acetate (2)

To a mixture of tert-butyl (R)-2-(5-((1-(dibenzo[b,d]furan-2-yl)ethyl)amino)-2-(methylthio)-6-oxopyrimidin-1(6H)-yl)acetate (1.1 g, 2.37 mmol) and (1-((2-(trimethylsilyl)ethoxy)methyl)-1H-pyrazol-4-yl)boronic acid (2.29 g, 9.46 mmol) in DMF (24 mL) was added CuTC (996 mg, 5.21 mmol) and Pd(PPh₃)₄ (274 mg, 0.24 mmol) at room temperature under N₂ atmosphere, the reaction mixture was degassed under N₂ atmosphere for three times and stirred at 90° C. for 48 hours in a sealed tube. The mixture was diluted with water and extracted with EtOAc twice. The combined organic layers were washed with saturated aq. NaHCO₃ solution and brine, dried over anhydrous Na₂SO₄, filtered, and concentrated under reduced pressure to dryness. The residue was purified by prep-TLC (PE:EtOAc=2:1) to give the title compound (250 mg, yield 17.1%) as a yellow solid. LC/MS (ESI) m/z: 616 (M+H)⁺.

### Step 2: Tert-butyl (R)-2-(5-((1-(dibenzo[b,d]furan-2-yl)ethyl)amino)-6-oxo-2-(1H-pyrazol-4-yl)pyrimidin-1(6H)-yl)acetate (3)

To a solution of tert-butyl (R)-2-(5-((1-(dibenzo[b,d]furan-2-yl)ethyl)amino)-6-oxo-2-(1-((2-(trimethylsilyl)ethoxy)methyl)-1H-pyrazol-4-yl)pyrimidin-1(6H)-yl)acetate (200 mg, 0.33 mmol) in THF (4 mL) was added TBAF (1.30 mL, 1.30 mmol, 1 M in THF) and the mixture was stirred at 55° C. for 16 hours. The mixture was diluted with water and extracted with EtOAc twice. The combined organic layers were washed with brine, dried over anhydrous Na₂SO₄, filtered, and concentrated under reduced pressure to dryness to give the title compound (157 mg, yield 99.6%) as a yellow oil, which was used directly in the next step without further purification. LC/MS (ESI) m/z: 486 (M+H)⁺.

### Step 3: (R)-2-(5-((1-(dibenzo[b,d]furan-2-yl)ethyl)amino)-6-oxo-2-(1H-pyrazol-4-yl)pyrimidin-1(6H)-yl)acetic acid (4)

To a solution of tert-butyl (R)-2-(5-((1-(dibenzo[b,d]furan-2-yl)ethyl)amino)-6-oxo-2-(1H-pyrazol-4-yl)pyrimidin-1(6H)-yl)acetate (157 mg, 0.32 mmol) in MeOH/1,4-dioxane/water (8 mL, 3/3/2) was added LiOH (31 mg, 1.28 mmol) and the reaction mixture was stirred at 75° C. overnight. The mixture was acidified with 1N aq. HCl to pH˜4 and extracted with EtOAc twice. The combined organic layers were dried over anhydrous Na₂SO₄, filtered, and concentrated under reduced pressure to give the title compound (130 mg, yield 93.6%) as a yellow solid, which was used directly in the next step without further purification. LC/MS (ESI) m/z: 430 (M+H)⁺.

### Step 4: (R)—N-((1H-pyrrolo[3,2-c]pyridin-2-yl)methyl)-2-(5-((1-(dibenzo[b,d]furan-2-yl)ethyl)amino)-6-oxo-2-(1H-pyrazol-4-yl)pyrimidin-1(6H)-yl)acetamide (Compound 360)

To a mixture of (R)-2-(5-((1-(dibenzo[b,d]furan-2-yl)ethyl)amino)-6-oxo-2-(1H-pyrazol-4-yl)pyrimidin-1(6H)-yl)acetic acid (130 mg, 0.30 mmol) and (1H-pyrrolo[3,2-c]pyridine-2-yl)methanamine hydrochloride (83 mg, 0.45 mmol) in DMF (4 mL) was added DIPEA (155 mg, 1.20 mmol) and HATU (137 mg, 0.36 mmol) and the reaction mixture was stirred at room temperature for 0.5 hour. The mixture was concentrated under reduced pressure to dryness. The residue was purified by prep-TLC (DCM:MeOH=6:1) and further purified by prep-HPLC to give Compound 360 (19.9 mg, yield 11.8%) as a white solid. ¹H NMR (400 MHz, DMSO-d₆) δ 13.18 (s, 1H), 11.44 (s, 1H), 8.86 (t, J=5.4 Hz, 1H), 8.75 (s, 1H), 8.20 (s, 1H), 8.11 (t, J=6.5 Hz, 2H), 7.88-7.62 (m, 4H), 7.60 (d, J=8.5 Hz, 1H), 7.51 (t, J=7.3 Hz, 1H), 7.39 (t, J=7.4 Hz, 1H), 7.32 (d, J=5.6 Hz, 1H), 6.95 (s, 1H), 6.37 (s, 1H), 5.76 (d, J=6.9 Hz, 1H), 4.78 (m, 1H), 4.70-4.61 (m, 2H), 4.47 (d, J=5.4 Hz, 2H), 1.59 (d, J=6.7 Hz, 3H). LC/MS (ESI) m/z: 559 (M+H)⁺. RT (Method A): 1.40 min.

### Scheme 112. Synthesis of (S*)—N-((1H-pyrrolo[3,2-c]pyridine-2-yl)methyl)-2-(5-(((R)-1-(dibenzo[b,d]furan-2-yl)ethyl)amino)-2-(2-fluorophenyl)-6-oxopyrimidin-1(6H)-yl)propenamide (Compound 363) and (R*)—N-((1H-pyrrolo[3,2-c]pyridine-2-yl)methyl)-2-(5-(((R)-1-(dibenzo[b,d]furan-2-yl)ethyl)amino)-2-(2-fluorophenyl)-6-oxopyrimidin-1(6H)-yl)propenamide (Compound 364)

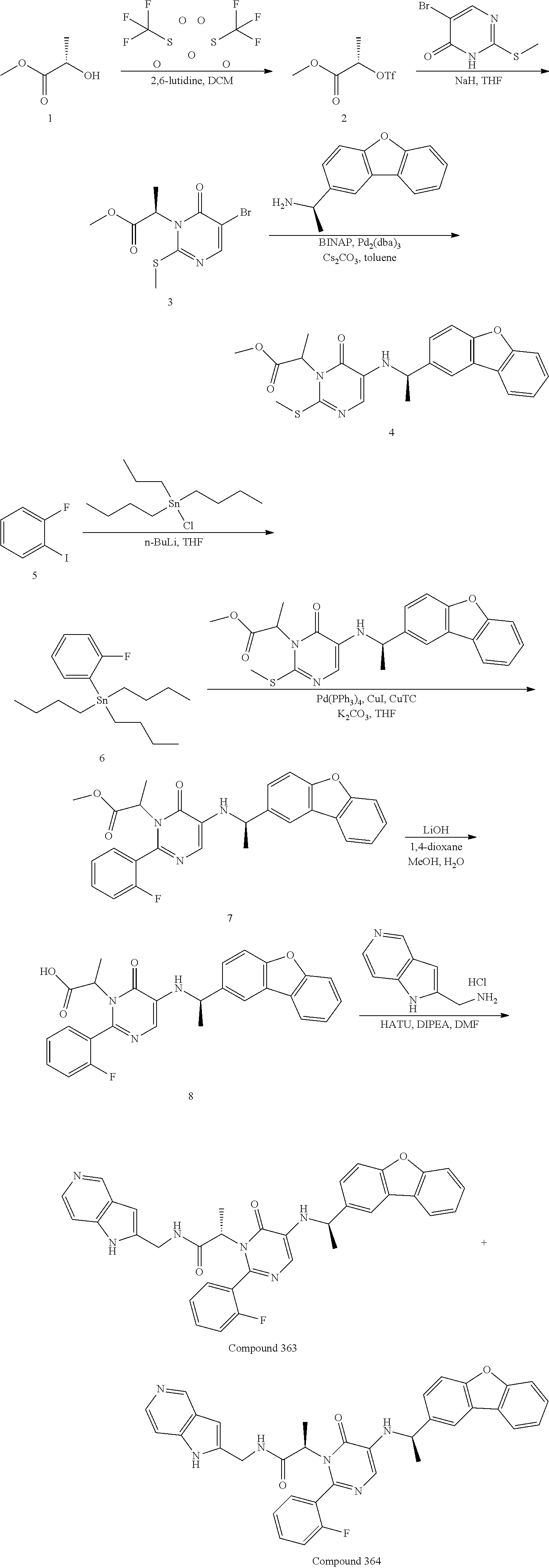

### Step 1: Methyl (S)-2-(((trifluoromethyl)sulfonyl)oxy)propanoate (2)

To a solution of methyl (S)-2-hydroxypropanoate (10 g, 96.15 mmol) in DCM (100 mL) was added 2,6-lutidine (10.80 g, 100.96 mmol) followed by dropwise addition of trifluoromethanesulfonic anhydride (28.47 g, 100.96 mmol) under N₂ atmosphere at 0° C. and the mixture was stirred at room temperature for 3 hours. The mixture was quenched with ice-water and extracted with DCM twice. The combined organic layers were washed with water and brine, dried over anhydrous Na₂SO₄, filtered, and concentrated under reduced pressure to dryness. The residue was purified by flash chromatography (silica gel, 0-20% EtOAc in PE) to give the title compound (16 g, yield 71.6%) as a colorless oil. LC/MS (ESI) m/z: 237 (M+H)⁺.

### Step 2: Methyl (R)-2-(5-bromo-2-(methylthio)-6-oxopyrimidin-1(6H)-yl)propanoate (3)

To a solution of 5-bromo-2-(methylthio)pyrimidin-4(3H)-one (10 g, 45.45 mmol) in THF (100 mL) was added NaH (1.73 g, 43.18 mmol, 60% dispersion in mineral oil) at 0° C. and the reaction was stirred at room temperature for 0.5 hour. Methyl (S)-2-(((trifluoromethyl)sulfonyl)oxy)propanoate (11.79 g, 50.00 mmol) was added to the above mixture at 0° C. and the resulting mixture was stirred at room temperature for 3 hours. The mixture was quenched with saturated aq. NH₄Cl solution at 0° C. and extracted with EtOAc twice. The combined organic layers were washed with water and brine, dried over anhydrous Na₂SO₄, filtered, and concentrated under reduced pressure to dryness. The residue was purified by flash chromatography (silica gel, 0-30% EtOAc in PE) to give the title compound (4.5 g, yield 32.24%) as a white solid. LC/MS (ESI) m/z: 307 (M+H)⁺.

### Step 3: Methyl 2-(5-(((R)-1-(dibenzo[b,d]furan-2-yl)ethyl)amino)-2-(methylthio)-6-oxopyrimidin-1(6H)-yl)propanoate (4)

To a mixture of methyl (R)-2-(5-bromo-2-(methylthio)-6-oxopyrimidin-1(6H)-yl)propanoate (2.5 g, 8.17 mmol) and (R)-1-(dibenzo[b,d]furan-2-yl)ethan-1-amine (2.24 g, 10.62 mmol) in toluene (30 mL) was added Cs₂CO₃ (7.99 g, 24.51 mmol), BINAP (509 mg, 0.81 mmol), Pd₂(dba)₃ (750 mg, 0.81 mmol) under N₂ atmosphere, the reaction mixture was degassed under N₂ atmosphere for three times and stirred at 100° C. overnight. The mixture was diluted with EtOAc, washed with brine, dried over anhydrous Na₂SO₄, filtered, and concentrated under reduced pressure to dryness. The residue was purified by flash chromatography (silica gel, 0-50% EtOAc in PE) to give the title compound (2.3 g, yield 64.28%) as a yellow solid. LC/MS (ESI) m/z: 438 (M+H)⁺.

### Step 4: Tributyl(2-fluorophenyl)stannane (6)

To a solution of 1-fluoro-2-iodobenzene (3 g, 13.51 mmol) in THF (30 mL) was added n-BuLi (6.8 mL, 17.56 mmol, 2.5 M in THF) dropwise under N₂ atmosphere at −70° C. and the mixture was stirred at this temperature for 0.5 hour. SnCl(n-Bu)₃ (8.80 g, 27.04 mmol) was added to the above mixture at −78° C. and the resulting mixture was stirred at −78° C. for 2 hours. The mixture was quenched with ice-water at 0° C. and extracted with EtOAc twice. The combined organic layers were washed with brine, dried over anhydrous Na₂SO₄, filtered, and concentrated under reduced pressure to dryness to give the title compound (4.5 g, yield 86.01%) as a colorless oil, which was used directly in the next step without further purification.

### Step 5: Methyl 2-(5-(((R)1-(dibenzo[b,d]furan-2-yl)ethyl)amino)-2-(2-fluorophenyl)-6-oxopyrimidin-1(6H)-yl)propanoate (7)

To a mixture of methyl 2-(5-(((R)-1-(dibenzo[b,d]furan-2-yl)ethyl)amino)-2-(methylthio)-6-oxopyrimidin-1(6H)-yl)propanoate (2.1 g, 4.80 mmol) and tributyl(2-fluorophenyl)stannane (9.19 g, 23.99 mmol) in THF (30 mL) was added K₂CO₃ (1.99 g, 14.4 mmol), CuI (98 mg, 0.51 mmol), CuTC (2.74 g 14.4 mmol) and Pd(PPh₃)₄ (556 mg, 0.48 mmol) under N₂ atmosphere, the reaction mixture was degassed under N₂ atmosphere for three times and stirred at 70° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-40% EtOAc in PE) to give the title compound (160 mg, yield 6.8%) as a white solid. LC/MS (ESI) m/z: 486 (M+H)⁺.

### Step 6: 2-(5-(((R)-1-(dibenzo[b,d]furan-2-yl)ethyl)amino)-2-(2-fluorophenyl)-6-oxopyrimidin-1(6H)-yl)propanoic acid (8)

To a solution of methyl 2-(5-(((R)-1-(dibenzo[b,d]furan-2-yl)ethyl)amino)-2-(2-fluorophenyl)-6-oxopyrimidin-1(6H)-yl)propanoate (160 mg, 0.32 mmol) in 1,4-dioxane/MeOH/water (2.5 mL, v/v/v=2/2/1) was added LiOH (12 mg, 0.49 mmol) and the reaction mixture was stirred at room temperature overnight. The mixture was acidified with 1 N aq. HCl to pH˜3 and extracted with EtOAc twice. The combined organic layers were dried over anhydrous Na₂SO₄, filtered, and concentrated under reduced pressure to give the title compound (150 mg, yield 96.7%) as a yellow oil, which was used directly in the next step without further purification. LC/MS (ESI) m/z: 472 (M+H)⁺.

### Step 7: (S*)—N-((1H-pyrrolo[3,2-c]pyridin-2-yl)methyl)-2-(5-(((R)-1-(dibenzo-[b,d]furan-2-yl)ethyl)amino)-2-(2-fluorophenyl)-6-oxopyrimidin-1(6H)-yl)propenamide (Compound 363) and (R*)—N-((1H-pyrrolo[3,2-c]pyridine-2-yl)methyl)-2-(5-(((R)-1-(dibenzo[b,d]furan-2-yl)ethyl)amino)-2-(2-fluorophenyl)-6-oxopyrimidin-1(6H)-yl)propenamide (Compound 364)

To a mixture of 2-(5-((I-1-(dibenzo[b,d]furan-2-yl)ethyl)amino)-2-(2-fluorophenyl)-6-oxopyrimidin-1(6H)-yl)propanoic acid (150 mg, 0.31 mmol) and (1H-pyrrolo[3,2-c]pyridine-2-yl)methanamine hydrochloride (87 mg, 0.47 mmol) in DMF (3 mL) was added HATU (182 mg, 0.47 mmol) and DIPEA (726 mg, 1.91 mmol) and the reaction mixture was stirred at room temperature for 1 hour. The mixture was diluted with EtOAc, washed with saturated aq. NH₄Cl solution and brine, the organic layer was dried over anhydrous Na₂SO₄, filtered, and concentrated under reduced pressure to dryness. The residue was purified by flash chromatography (silica gel, 0-11% MeOH in DCM) and further purified by chiral SFC (ChiralCel OJ, 250×21.2 mm, A for CO₂ and 30% B for MEOH+0.1% MEA, 60 mL/min) to give Compound 363 (ChiralCel OJ, 100×4.6 mm, A for CO₂ and 20% B for MEOH+0.05% DEA, retention time: 5.561 min) 26.0 mg, yield 13.6%) and Compound 364 (retention time: 6.764 min, 25.6 mg, yield 13.4%) as a white solid. Compound 363: ¹H NMR (400 MHz, CD₃OD) δ 8.77 (s, 1H), 8.10 (d, J=3.0 Hz, 1H), 8.00-7.88 (m, 1H), 7.57-7.48 (m, 4H), 7.47-7.37 (m, 4H), 7.31-7.19 (m, 3H), 6.93 (d, J=10.5 Hz, 1H), 6.58 (s, 1H), 4.77 (m, 1H), 4.70-4.64 (m, 1H), 4.60-4.42 (m, 2H), 1.75-1.57 (m, 3H), 1.69 (d, J=6.2 Hz, 3H). LC/MS (ESI) m/z: 601 (M+H)⁺. RT (Method A): 1.75 min. Compound 364: ¹H NMR (400 MHz, CD₃OD) δ 9.02 (s, 1H), 8.30 (d, J=5.3 Hz, 1H), 8.09 (s, 1H), 8.03 (d, J=7.3 Hz, 1H), 7.98-7.89 (m, 1H), 7.66-7.51 (m, 5H), 7.50-7.44 (m, 1H), 7.38-7.32 (m, 1H), 7.31-7.20 (m, 2H), 6.95 (s, 2H), 4.71-4.64 (m, 3H), 4.62-4.55 (m, 1H), 1.81-1.60 (m, 3H), 1.66 (d, J=6.0 Hz, 3H). LC/MS (ESI) m/z: 601 (M+H)⁺. RT (Method A): 1.80 min.

### Scheme 113. Synthesis of (R)—N-((1H-pyrrolo[3,2-c]pyridine-2-yl)methyl)-2-(5-((1-(dibenzo[b,d]furan-2-yl)ethyl)amino)-2-(methoxymethyl)-6-oxopyrimidin-1(6H)-yl)acetamide (Compound 365)

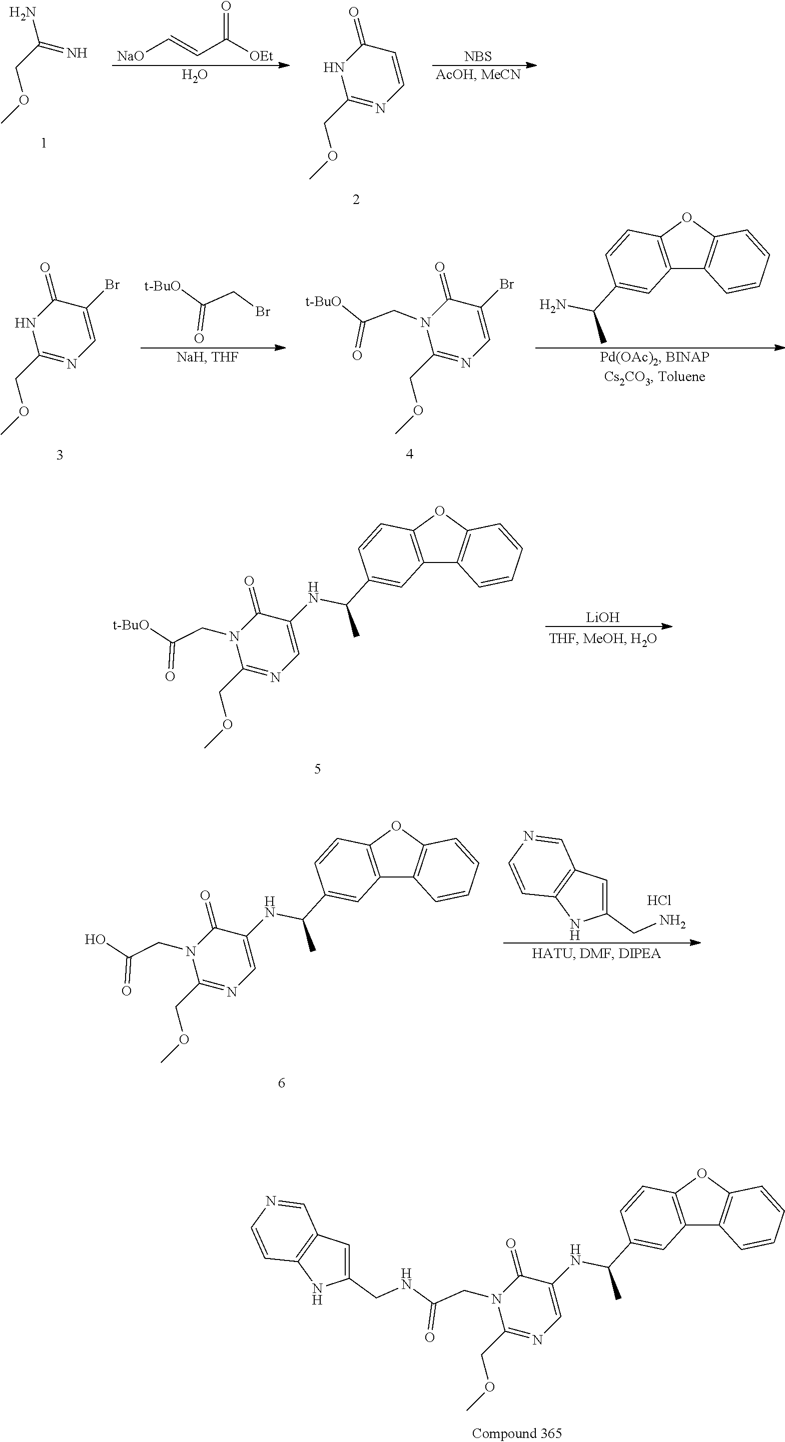

### Step 1: 2-(Methoxymethyl)pyrimidin-4(3H)-one (2)

To a mixture of 2-methoxyacetimidamide (300 mg, 2.41 mmol) in water (3 mL) was added sodium (E)-3-ethoxy-3-oxoprop-1-en-1-olate (499 mg, 3.61 mmol) and the reaction mixture was stirred at room temperature overnight. The mixture was filtered, and the filter cake was dried under vacuum to dryness. The residue was purified by flash chromatography (silica gel, 0-10% MeOH in DCM) to give the title compound (100 mg, yield 29.61%) as a white solid. LC/MS (ESI) m/z: 141 (M+H)⁺.

### Step 2: 5-Bromo-2-(methoxymethyl)pyrimidin-4(3H)-one (3)

To a mixture of 2-(methoxymethyl)pyrimidin-4(3H)-one (130 mg, 0.93 mmol) in MeCN (2.5 mL) was added NBS (198 mg, 1.11 mmol) and AcOH (0.5 mL) and the reaction mixture was stirred at room temperature for 2 hours. The mixture was concentrated under reduced pressure to dryness. The residue was purified by flash chromatography (silica gel, 0-5% MeOH in DCM) to give the title compound (150 mg, yield 73.82%) as a white solid. LC/MS (ESI) m/z: 219 (M+H)⁺.

### Step 3: Tert-butyl 2-(5-bromo-2-(methoxymethyl)-6-oxopyrimidin-1(6H)-yl)acetate (4)

To a solution of 5-bromo-2-(methoxymethyl)pyrimidin-4(3H)-one (150 mg, 0.68 mmol) in THF (5 ml) was added NaH (822 mg, 2.06 mmol, 60% dispersion in mineral oil) and the reaction mixture was stirred at 60° C. for 0.5 hour. Then tert-butyl 2-bromoacetate (400 mg, 2.05 mmol) was added to the reaction mixture and the resulting mixture was stirred at 60° C. for 16 hours. The reaction mixture was quenched with saturated aq. NH₄Cl solution and extracted with EtOAc twice. The combined organic layers were washed with water and brine, dried over anhydrous Na₂SO₄, filtered, and concentrated to dryness under reduce pressure. The residue was purified by flash chromatography (silica gel, 0-25% EtOAc in PE) to give the title compound (130 mg, yield 56.98%) as a white solid. LC/MS (ESI) m/z: 335 (M+H)⁺.

### Step 4: Tert-butyl (R)-2-(5-((1-(dibenzo[b,d]furan-2-yl)ethyl)amino)-2-(methoxymethyl)-6-oxopyrimidin-1(6H)-yl)acetate (5)

To a mixture of tert-butyl 2-(5-bromo-2-(methoxymethyl)-6-oxopyrimidin-1(6H)-yl)acetate (130 mg, 0.39 mmol) and (R)-1-(dibenzo[b,d]furan-2-yl)ethan-1-amine (118 mg, 0.56 mmol) in toluene (5 mL) was added Cs₂CO₃ (498 mg, 1.53 mmol), Brettphos (64 mg, 0.10 mmol) and Pd(OAc)₂ (12 mg, 0.05 mmol) under N₂ atmosphere and the reaction mixture was stirred at 100° C. for 3 hours. The mixture was diluted with EtOAc, washed with water and brine, dried over anhydrous Na₂SO₄, filtered, and concentrated under reduced pressure to dryness. The residue was purified by flash chromatography (silica gel, 10-50% EtOAc in PE) to give the title compound (150 mg, yield 63.54%) as a yellow solid. LC/MS (ESI) m/z: 464 (M+H)⁺.

### Step 5: (R)-2-(5-((1-(dibenzo[b,d]furan-2-yl)ethyl)amino)-2-(methoxymethyl)-6-oxopyrimidin-1(6H)-yl)acetic acid (6)

To a solution of tert-butyl (R)-2-(5-((1-(dibenzo[b,d]furan-2-yl)ethyl)amino)-2-(methoxymethyl)-6-oxopyrimidin-1(6H)-yl)acetate (150 mg, 0.32 mmol) in THF/MeOH/water (2.0 mL, v/v/v=1/2/1) was added LiOH (77.75 mg, 3.20 mmol) and the reaction mixture was stirred at 60° C. for 16 hours. The mixture was acidified with 1 N aq. HCl to pH˜5 and extracted with EtOAc twice. The combined organic layers were dried over anhydrous Na₂SO₄, filtered, and concentrated under reduced pressure to give crude title compound (170 mg, 100% yield) as a yellow solid, which was used directly in the next step without further purification. LC/MS (ESI) m/z: 408 (M+H)⁺.

### Step 6: (R)—N-((1H-pyrrolo[3,2-c]pyridine-2-yl)methyl)-2-(5-((1-(dibenzo[b,d]furan-2-yl)ethyl)amino)-2-(methoxymethyl)-6-oxopyrimidin-1(6H)-yl)acetamide (Compound 365)

To a mixture of crude I-2-(5-((1-(dibenzo[b,d]furan-2-yl)ethyl)amino)-2-(methoxymethyl)-6-oxopyrimidin-1(6H)-yl)acetic acid (170 mg, 0.32 mmol) and (1H-pyrrolo[3,2-c]pyridine-2-yl)methanamine hydrochloride (103 mg, 0.56 mmol) in DMF (4 mL) was added DIPEA (238 mg, 1.85 mmol) and HATU (168 mg, 0.44 mmol) and the reaction mixture was stirred at room temperature for 1 hour. The mixture was concentrated under reduced pressure to dryness. The residue was purified by prep-HPLC to give Compound 365 (60 mg, yield 30%) as a white solid. ¹H NMR (400 MHz, DMSO-d₆) δ 11.36 (s, 1H), 8.78 (t, J=5.6 Hz, 1H), 8.74 (s, 1H), 8.18 (s, 1H), 8.13-8.08 (m, 2H), 7.68 (d, J=8.2 Hz, 1H), 7.66-7.63 (m, 1H), 7.58 (d, J=8.7 Hz, 1H), 7.51 (t, J=7.8 Hz, 1H), 7.39 (t, J=7.6 Hz, 1H), 7.32 (d, J=5.7 Hz, 1H), 6.85 (s, 1H), 6.43 (s, 1H), 5.90 (d, J=7.0 Hz, 1H), 4.77 (m, 2H), 4.62 (m, 1H), 4.46 (d, J=5.4 Hz, 2H), 4.22 (s, 2H), 3.14 (s, 3H), 1.58 (d, J=6.7 Hz, 3H). LC/MS (ESI) m/z: 537 (M+H)⁺. RT (Method A): 1.49 min.

### Scheme 114. Synthesis of (R)—N-((1H-pyrrolo[3,2-c]pyridine-2-yl)methyl)-2-(5-((1-(dibenzo[b,d]furan-2-yl)ethyl)amino)-3-methyl-2,6-dioxo-3,6-dihydropyrimidin-1(2H)-yl)acetamide (Compound 366)

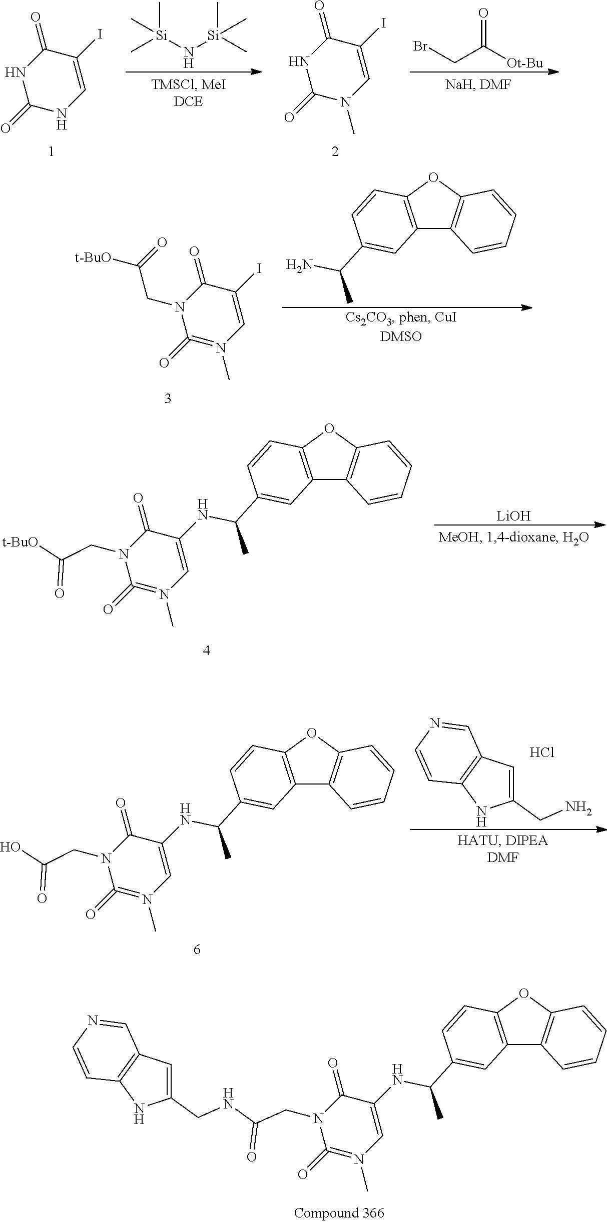

### Step 1: 5-Iodo-1-methylpyrimidine-2,4(1H,3H)-dione (2)

To a solution of 5-iodopyrimidine-2,4(1H,3H)-dione (10.0 g, 42.02 mmol) in DCE (130 mL) was added bis(trimethylsilyl)amine (17.0 g, 105.0 mmol) and TMSCl (2.28 g, 21.00 mmol) under N₂ atmosphere. The mixture was degassed under N₂ atmosphere for three times and stirred at 87° C. for 3 hours. The mixture was concentrated under reduced pressure to dryness. The residue was dissolved in DCE (130 mL) and MeI (22.66 g, 159.60 mmol) and I₂ (110 mg, 0.43 mmol) was added, the resulting mixture was stirred in a sealed tube at 87° C. overnight. The reaction mixture was concentrated under reduced pressure to dryness, the residue was purified by flash chromatography (silica gel, 0-15% MeOH in DCM) to give the title compound (10.0 g, yield 94.4%) as a yellow solid. ¹H NMR (400 MHz, DMSO-d₆) δ 8.20 (s, 1H), 3.24 (s, 3H). LC/MS (ESI) m/z: 253/255 (M+H)⁺.

### Step 2: Tert-butyl 2-(5-iodo-3-methyl-2,6-dioxo-3,6-dihydropyrimidin-1(2H)-yl)acetate (3)

To a solution of 5-iodo-1-methylpyrimidine-2,4(1H,3H)-dione (8.0 g, 31.75 mmol) in THF (100 mL) was added NaH (2.54 g, 63.50 mmol, 60% dispersion in mineral oil) in portions at 0° C. under N₂ atmosphere and the mixture was stirred at 0° C. for 30 minutes. Then tert-butyl 2-bromoacetate (18.57 g, 95.24 mmol) was added to the reaction mixture and the resulting mixture was stirred at room temperature overnight. The mixture was quenched with saturated aq. NH₄Cl solution at 0° C. and extracted wit EtOAc twice. The combined organic layers were washed with brine, dried over anhydrous na₂SO₄, filtered, and concentrated under reduced pressure to dryness. The residue was purified by flash chromatography (silica gel, 0-22% EtOAc in PE) to give the title compound (5.38 g, yield 45.9%) as a yellow solid. ¹H NMR (400 MHz, DMSO-d₆) δ 8.32 (s, 1H), 4.48 (s, 2H), 3.32 (s, 3H), 1.41 (s, 9H). LC/MS (ESI) m/z: 311 (M−56)⁺.

### Step 3: Tert-butyl (R)-2-(5-((1-(dibenzo[b,d]furan-2-yl)ethyl)amino)-3-methyl-2,6-dioxo-3,6-dihydropyrimidin-1(2H)-yl)acetate (4)

To a mixture of tert-butyl 2-(5-iodo-3-methyl-2,6-dioxo-3,6-dihydropyrimidin-1(2H)-yl)acetate (2.0 g, 5.46 mmol) and (R)-1-(dibenzo[b,d]furan-2-yl)ethan-1-amine (1.35 g, 5.45 mmol) in DMSO (25 mL) was added Cs₂CO₃ (4.44 g, 13.62 mmol), CuI (1.04 g, 5.47 mmol) and o-Phenanthrolin (1.74 g, 8.20 mmol) under N₂ atmosphere and the mixture was stirred in a sealed tube at 70° C. for 5 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-43% EtOAc in PE) to give the title compound (430 mg, yield 17.51%) as a yellow oil. ¹H NMR (400 MHz, CDCl₃) δ 7.98-7.91 (m, 2H), 7.58-7.51 (m, 2H), 7.48-7.44 (m, 1H), 7.42-7.39 (m, 1H), 7.37-7.32 (m, 1H), 5.79 (s, 1H), 4.65 (s, 2H), 4.29-4.22 (m, 1H), 3.10 (s, 3H), 1.58 (d, J=6.7 Hz, 3H), 1.48 (s, 9H). LC/MS (ESI) m/z: 450 (M+H)⁺.

### Step 4: (R)-2-(5-((1-(dibenzo[b,d]furan-2-yl)ethyl)amino)-3-methyl-2,6-dioxo-3,6-dihydropyrimidin-1(2H)-yl)acetic acid (5)

To a solution of tert-butyl (R)-2-(5-((1-(dibenzo[b,d]furan-2-yl)ethyl)amino)-3-methyl-2,6-dioxo-3,6-dihydropyrimidin-1(2H)-yl)acetate (400 mg, 0.89 mmol) in MeOH/1,4-dioxane/water (5 mL, 2/2/1) was added LiOH (100 mg, 4.17 mmol) and the reaction mixture was stirred at 60° C. overnight. The mixture was acidified with 1 N aq. HCl to pH˜3 and extracted with EtOAc twice. The combined organic layers were dried over anhydrous Na₂SO₄, filtered, and concentrated under reduced pressure. The residue was purified by prep-TLC (DCM:MeOH=10:1) to give the title compound (130 mg, yield 37.1%) as a white solid. ¹H NMR (400 MHz, CD₃OD) δ 8.06-8.00 (m, 2H), 7.59-7.44 (m, 4H), 7.35 (t, J=7.5 Hz, 1H), 6.25 (s, 1H), 4.62 (s, 2H), 4.47-4.40 (m, 1H), 3.13 (s, 3H), 1.58 (d, J=6.7 Hz, 3H). LC/MS (ESI) m/z: 394 (M+H)⁺.

### Step 5: (R)—N-((1H-pyrrolo[3,2-c]pyridine-2-yl)methyl)-2-(5-((1-(dibenzo[b,d]furan-2-yl)ethyl)amino)-3-methyl-2,6-dioxo-3,6-dihydropyrimidin-1(2H)-yl)acetamide (Compound 366)

To a mixture of (R)-2-(5-((1-(dibenzo[b,d]furan-2-yl)ethyl)amino)-3-methyl-2,6-dioxo-3,6-dihydropyrimidin-1(2H)-yl)acetic acid (130 mg, 0.33 mmol) and (1H-pyrrolo[3,2-c]pyridine-2-yl)methanamine hydrochloride (84 mg, 0.46 mmol) in DMF (3 mL) was added DIPEA (196 mg, 1.52 mmol) and HATU (173 mg, 0.46 mmol) and the reaction mixture was stirred at room temperature for 30 minutes. The mixture was concentrated under reduced pressure to dryness. The residue was purified by flash chromatography (silica gel, 0-13% MeOH in MeOH) and further purified by prep-HPLC to give Compound 366 (70 mg, yield 40.53%) as a white solid. ¹H NMR (400 MHz, DMSO-d₆) δ 11.34 (s, 1H), 8.73 (s, 1H), 8.66 (t, J=5.5 Hz, 1H), 8.16 (d, J=1.5 Hz, 1H), 8.12-8.07 (m, 2H), 7.69-7.63 (m, 2H), 7.58-7.49 (m, 2H), 7.39 (t, J=7.5 Hz, 1H), 7.31 (d, J=5.7 Hz, 1H), 6.49 (s, 1H), 6.40 (s, 1H), 4.97 (d, J=7.5 Hz, 1H), 4.51 (d, J=2.7 Hz, 2H), 4.45-4.40 (m, 3H), 3.12 (s, 3H), 1.53 (d, J=6.7 Hz, 3H). LC/MS (ESI) m/z: 523 (M+H)⁺. RT (Method A): 1.47 min.

### Scheme 115. Synthesis of (R)—N-((1H-pyrrolo[3,2-c]pyridin-2-yl)methyl)-2-(5-((1-(dibenzo[b,d]furan-2-yl)ethyl)amino)-6-oxo-2-(pyrrolidin-1-yl)pyrimidin-1(6H)-yl)acetamide (Compound 367)

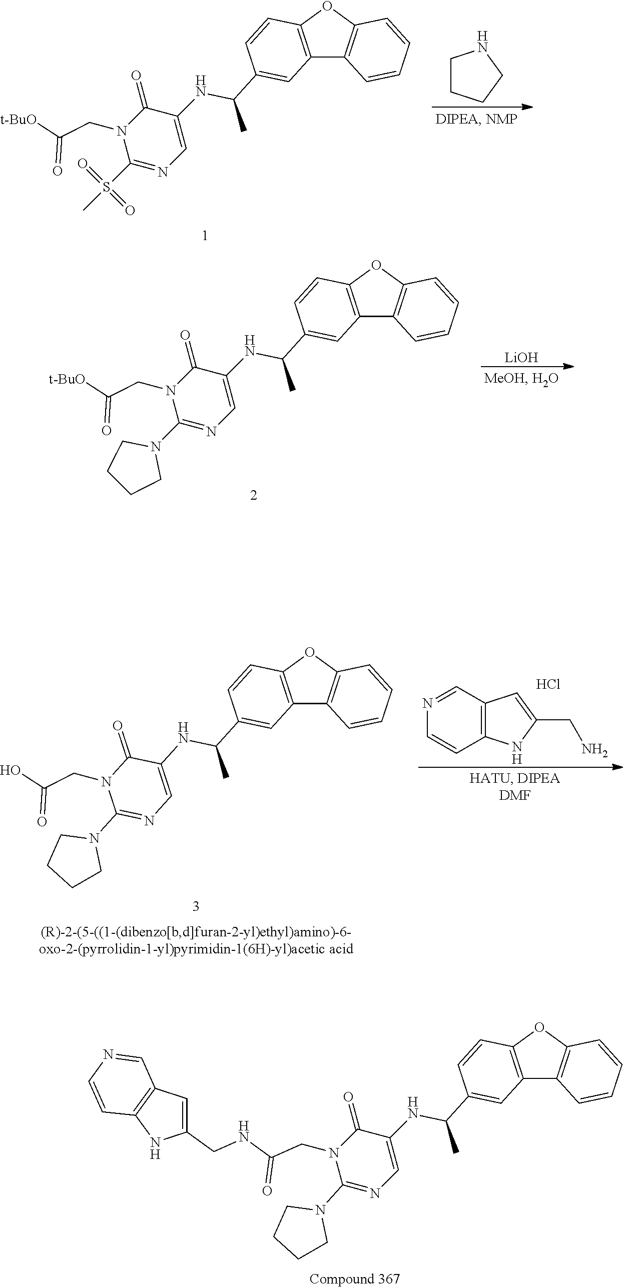

### Step 1: tert-butyl (R)-2-(5-((1-(dibenzo[b,d]furan-2-yl)ethyl)amino)-6-oxo-2-(pyrrolidin-1-yl)pyrimidin-1(6H)-yl)acetate (2)

To a mixture of tert-butyl (R)2-(5-((1-(dibenzo[b,d]furan-2-yl)ethyl)amino)-2-(methylsulfonyl)-6-oxopyrimidin-1(6H)-yl)acetate (220 mg, 0.44 mmol) and pyrrolidine (157 mg, 2.21 mmol) in NMP (3 mL) was added DIPEA (160 mg, 1.24 mmol) under N₂ atmosphere. The mixture was degassed under N₂ atmosphere for three times and stirred in a sealed tube at 150° C. for 30 minutes. The reaction mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-15% EtOAc in PE) to give the title compound (180 mg, yield 83.3%) as a colorless oil. LC/MS (ESI) m/z: 489 (M+H)⁺.

### Step 2: (R)-2-(5-((1-(dibenzo[b,d]furan-2-yl)ethyl)amino)-6-oxo-2-(pyrrolidin-1-yl)pyrimidin-1(6H)-yl)acetic acid (3)

To a solution of tert-butyl (R)-2-(5-((1-(dibenzo[b,d]furan-2-yl)ethyl)amino)-6-oxo-2-(pyrrolidin-1-yl)pyrimidin-1(6H)-yl)acetate (2) (180 mg, 0.37 mmol) in MeOH/water (6 mL, v/v=2/1) was added LiOH (35 mg, 1.33 mmol) and the mixture was stirred at 60° C. for three hours. The mixture was acidified with 1 N aq. HCl to pH˜5 and extracted with EtOAc twice. The combined organic layers were washed with brine, dried over anhydrous Na₂SO₄, filtered, and concentrated under reduced pressure to dryness to give the title compound (159 mg, yield 99.6%) as a yellow solid, which was used directly in the next step without further purification. LC/MS (ESI) m/z: 433 (M+H)⁺.

### Step 3: (R)—N-((1H-pyrrolo[3,2-c]pyridin-2-yl)methyl)-2-(5-((1-(dibenzo[b,d]furan-2-yl)ethyl)amino)-6-oxo-2-(pyrrolidin-1-yl)pyrimidin-1(6H)-yl)acetamide (Compound 367)

To a mixture of (R)-2-(5-((1-(dibenzo[b,d]furan-2-yl)ethyl)amino)-6-oxo-2-(pyrrolidin-1-yl)pyrimidin-1(6H)-yl)acetic acid (159 mg, 0.37 mmol) and (1H-pyrrolo[3,2-c]pyridine-2-yl)methanamine hydrochloride (115 mg, 0.63 mmol) in DMF (2 mL) was added DIPEA (224 mg, 1.74 mmol) and HATU (198 mg, 0.52 mmol), the reaction mixture was stirred at room temperature for 1 hour. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-10% MeOH in DCM) and further purified by prep-HPLC to give Compound 367 (24.8 mg, yield 12.0%) as a white solid. ¹H NMR (400 MHz, DMSO-d₆) δ 11.31 (s, 1H), 8.73 (s, 2H), 8.16 (s, 1H), 8.10 (t, J=6.4 Hz, 2H), 7.68 (d, J=8.2 Hz, 1H), 7.64 (d, J=8.5 Hz, 1H), 7.56 (d, J=8.5 Hz, 1H), 7.50 (t, J=7.8 Hz, 1H), 7.38 (t, J=7.4 Hz, 1H), 7.32 (d, J=5.6 Hz, 1H), 6.69 (s, 1H), 6.43 (s, 1H), 5.18 (d, J=6.6 Hz, 1H), 4.68-4.59 (m, 2H), 4.55-4.49 (m, 1H), 4.47 (d, J=5.3 Hz, 2H), 3.13-3.08 (m, 4H), 1.68-1.63 (m, 4H), 1.54 (d, J=6.6 Hz, 3H). LC/MS (ESI) m/z: 562 (M+H)⁺. RT (Method A): 1.64 min.

The following compounds were prepared based on Scheme 115:

| # | Reactant A | Reactant B | Reactant C | Characterization Data |
|---|---|---|---|---|
| 368 | 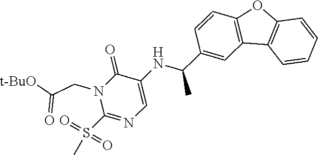 | 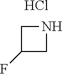 | 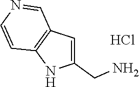 [SMILES: NCc1cc2cnccc2[nH]1 ; Cl] | ¹H NMR (400 MHz, DMSO-d₆) δ 11.35 (s, 1H), 8.78 (s, 1H), 8.74 (s, 1H), 8.16 (s, 1H), 8.10 (t, J = 7.5 Hz, 2H), 7.68 (d, J = 8.0 Hz, 1H), 7.63 (d, J = 8.5 Hz, 1H), 7.56 (d, J = 8.7 Hz, 1H), 7.51 (t, J = 7.7 Hz, 1H), 7.39 (t, J = 7.5 Hz, 1H), 7.32 (d, J = 5.6 Hz, 1H), 6.73 (s, 1H), 6.45 (s, 1H), 5.34-5.16 (m, 2H), 4.59-4.54 (m, 1H), 4.53-4.45 (m, 4H), 4.19-4.09 (m, 2H), 3.96-3.85 (m, 2H), 1.55 (d, J = 6.7 Hz, 3H). LC/MS (ESI) m/z: 566 (M + H)⁺. RT (Method A): 1.64 min. |
| 369 | 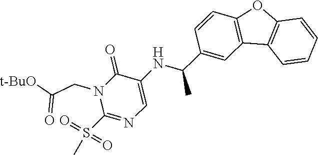 [SMILES: C[C@@H](Nc1cnc(S(C)(=O)=O)n(CC(=O)OC(C)(C)C)c1=O)c1ccc2oc3ccccc3c2c1] | 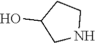 | 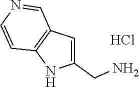 [SMILES: NCc1cc2cnccc2[nH]1 ; Cl] | ¹H NMR (400 MHz, DMSO-d₆) δ 11.35 (s, 1H), 8.76 (d, J = 5.7 Hz, 1H), 8.74 (s, 1H), 8.16 (s, 1H), 8.11 (t, J = 6.3 Hz, 2H), 7.68 (d, J = 8.2 Hz, 1H), 7.64 (d, J = 8.5 Hz, 1H), 7.56 (d, J = 8.5 Hz, 1H), 7.51 (t, J = 7.8 Hz, 1H), 7.39 (t, J = 7.5 Hz, 1H), 7.32 (d, J = 5.5 Hz, 1H), 6.69 (s, 1H), 6.44 (s, 1H), 5.15 (t, J = 6.9 Hz, 1H), 4.87 (s, 1H), 4.67-4.60 (m, 1H), 4.60 (d, J = 3.2 Hz, 1H), 4.51 (dd, J = 6.7, 3.3 Hz, 1H), 4.48 (d, J = 5.2 Hz, 2H), |
| | | | | 4.17 (s, 1H), 3.36 (m, 2H), 3.12-3.07 (m, |
| | | | | 1H), 2.95-2.91 (m, 1H), 1.81-1.73 (m, |
| | | | | 1H), 1.64-1.59 (m, 1H), 1.54 (d, J = 6.7 |
| | | | | Hz, 3H). LC/MS (ESI) m/z: 578 (M + H)⁺. |
| | | | | RT (Method A): 1.41 min. |
| 370 | 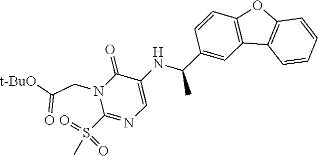 [SMILES: C[C@@H](Nc1cnc(S(C)(=O)=O)n(CC(=O)OC(C)(C)C)c1=O)c1ccc2oc3ccccc3c2c1] | 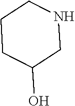 | 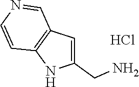 [SMILES: NCc1cc2cnccc2[nH]1 ; Cl] | ¹H NMR (400 MHz, CD₃OD) δ 8.77 (s, 1H), 8.14 (d, J = 6.0 Hz, 1H), 8.04 (s, 1H), 7.98 (d, J = 7.6 Hz, 1H), 7.55 (d, J = 8.3 Hz, 1H), 7.52 (s, 1H), 7.51 (s, 1H), 7.48 (s, 1H), 7.45 (d, J = 8.3 Hz, 1H), 7.33 (t, J = 7.4 Hz, 1H), 6.72 (s, 1H), 6.66 (s, 1H), 4.94-4.88 (m, 1H), 4.83-4.78 (m, 1H), 4.64 (s, 2H), 4.59 (d, J = 7.0 Hz, 1H), 3.74-3.64 (m, 1H), 3.10- 3.01 (m, 1H), 2.94-2.85 (m, 1H), 2.79- 2.57 (m, 2H), 1.84-1.68 (m, 2H), 1.63 (d, |
| | | | | J = 6.7 Hz, 3H), 1.47-1.35 (m, 2H). LC/MS |
| | | | | (ESI) m/z: 592 (M + H)⁺. RT (Method A): |
| | | | | 1.52 min. |
| 371a | 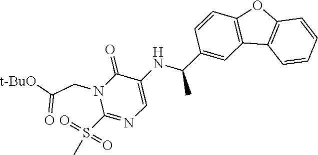 | 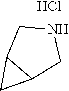 | 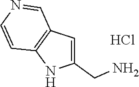 [SMILES: NCc1cc2cnccc2[nH]1 ; Cl] | ¹H NMR (400 MHz, CD₃OD) δ 8.71 (s, 1H), 8.11 (d, J = 5.8 Hz, 1H), 8.03 (s, 1H), 7.97 (d, J = 7.7 Hz, 1H), 7.53 (m, 3H), 7.47 (d, J = 7.4 Hz, 1H), 7.40 (d, J = 5.7 Hz, 1H), 7.33 (t, J = 7.5 Hz, 1H), 6.71 (s, 1H), 6.58 (s, 1H), 4.70 (s, 2H), 4.61 (s, 2H), 4.57-4.54 (m, 1H), 3.26-3.21 (m, 2H), 3.17-3.09 (m, 2H), 1.62 (d, J = 6.7 Hz, 3H), 1.39-1.36 (m, 2H), 0.38 (t, J = 5.8 Hz, 2H). LC/MS (ESI) m/z: 574 (M + H)⁺. RT (Method A): 1.78 min. |
| 373 | 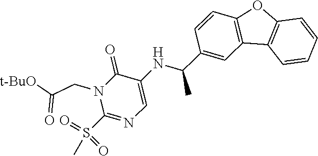 | 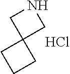 | 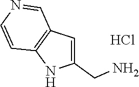 [SMILES: NCc1cc2cnccc2[nH]1 ; Cl] | ¹H NMR (400 MHz, DMSO-d₆) δ 11.30 (s, 1H), 8.76-8.69 (m, 2H), 8.15 (s, 1H), 8.09 (d, J = 7.1 Hz, 2H), 7.68 (d, J = 8.3 Hz, 1H), 7.63 (d, J = 8.5 Hz, 1H), 7.56 (s, 1H), 7.50 (s, 1H), 7.38 (s, 1H), 7.33 (d, J = 5.4 Hz, 1H), 6.69 (s, 1H), 6.43 (s, 1H), 5.06 (d, J = 6.7 Hz, 1H), 4.54-4.41 (m, 5H), 3.74-3.68 (m, 4H), 1.94-1.88 (m, 4H), 1.63-1.56 (m, 2H), 1.53 (d, J = 6.6 Hz, 3H). LC/MS (ESI) m/z: 588 (M + H)⁺. RT (Method A): 1.82 min. |
| 374 | 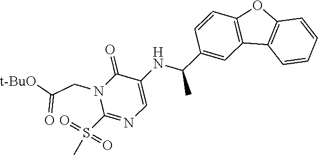 | 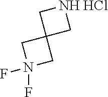 | 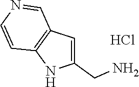 [SMILES: NCc1cc2cnccc2[nH]1 ; Cl] | ¹H NMR (400 MHz, CD₃OD) δ 8.70 (s, 1H), 8.10 (d, J = 5.6 Hz, 1H), 8.00 (s, 1H), 7.94 (d, J = 7.7 Hz, 1H), 7.55-7.51 (m, 1H), 7.50- 7.41 (m, 3H), 7.37 (d, J = 5.8 Hz, 1H), 7.31 (t, J= 7.5 Hz, 1H), 6.71 (s, 1H), 6.55 (s, 1H), 4.64 (d, J = 4.7 Hz, 2H), 4.60 (s, 2H), 4.54- 4.48 (m, 1H), 3.89-3.83 (m, 4H), 2.53 (t, J = 12.1 Hz, 4H), 1.58 (d, J = 6.7 Hz, 3H). LC/MS (ESI) m/z: 624 (M + H)⁺. RT (Method A): 1.82 min. |
| 379^{b} | 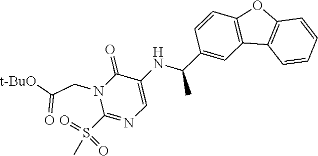 | 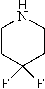 | 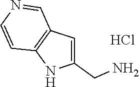 [SMILES: NCc1cc2cnccc2[nH]1 ; Cl] | ¹H NMR (400 MHz, DMSO-d₆) δ 11.44 (s, 1H), 8.93 (t, J = 5.4 Hz, 1H), 8.73 (s, 1H), 8.19 (s, 1H), 8.10 (t, J = 6.0 Hz, 2H), 7.70- 7.63 (m, 2H), 7.61-7.57 (m, 1H), 7.51 (t, J = 7.7 Hz, 1H), 7.39 (t, J = 7.5 Hz, 1H), 7.32 (d, J = 5.6 Hz, 1H), 6.74 (s, 1H), 6.44 (s, 1H), 5.56 (d, J = 6.8 Hz, 1H), 4.76-4.64 (m, 2H), 4.55 (dd, J= 13.5, 6.8 Hz, 1H), 4.46 (t, J = 10.9 Hz, 2H), 2.92-2.84 (m, 4H), 1.95-1.84 (m, 4H), 1.56 (d, J = 6.7 Hz, 3H). LC/MS (ESI) m/z: 612 (M + H)⁺. RT (Method A): 1.85 min. |
| 380^{b} | 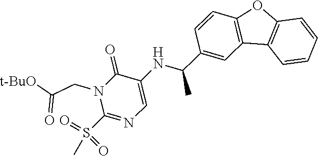 [SMILES: C[C@@H](Nc1cnc(S(C)(=O)=O)n(CC(=O)OC(C)(C)C)c1=O)c1ccc2oc3ccccc3c2c1] | 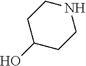 | 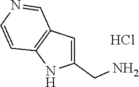 [SMILES: NCc1cc2cnccc2[nH]1 ; Cl] | ¹H NMR (400 MHz, DMSO-d₆) δ 11.32 (s, 1H), 8.80-8.76 (m, 1H), 8.73 (s, 1H), 8.17 (d, J = 1.1 Hz, 1H), 8.10 (t, J = 6.2 Hz, 2H), 7.69-7.63 (m, 2H), 7.59-7.56 (m, 1H), 7.53-7.49 (m, 1H), 7.39 (t, J = 7.6 Hz, 1H), 7.32 (d, J = 5.4 Hz, 1H), 6.72 (s, 1H), 6.43 (s, 1H), 5.43 (d, J = 6.7 Hz, 1H), 4.65 (d, J = 5.2 Hz, 1H), 4.61 (d, J = 3.9 Hz, 1H), 4.56- 4.51 (m, 1H), 4.47 (d, J = 5.7 Hz, 2H), 3.53- |
| | | | | 3.46 (m, 1H), 3.00-2.93 (m, 2H), 2.61- |
| | | | | 2.54 (m, 2H), 2.43 (s, 1H), 1.70-1.63 (m, |
| | | | | 2H), 1.55 (d, J = 6.5 Hz, 3H), 1.45-1.36 |
| | | | | (m, 2H). LC/MS (ESI) m/z: 592 (M + H)⁺. |
| | | | | RT (Method A): 1.46 min. |
| 381 | 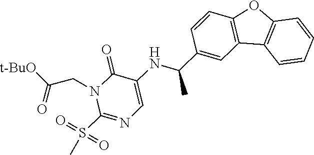 [SMILES: C[C@@H](Nc1cnc(S(C)(=O)=O)n(CC(=O)OC(C)(C)C)c1=O)c1ccc2oc3ccccc3c2c1] | 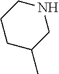 | 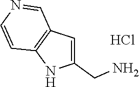 [SMILES: NCc1cc2cnccc2[nH]1 ; Cl] | ¹H NMR (400 MHz, DMSO-d₆) δ 11.31 (s, 1H), 8.76 (t, J = 5.4 Hz, 1H), 8.72 (s, 1H), 8.17 (s, 1H), 8.12-8.08 (m, 2H), 7.66 (dd, J = 15.5, 8.3 Hz, 2H), 7.58 (d, J = 8.5 Hz, 1H), 7.51 (t, J = 7.8 Hz, 1H), 7.39 (t, J = 7.5 Hz, 1H), 7.32 (d, J = 5.6 Hz, 1H), 6.73 (s, 1H), 6.42 (s, 1H), 5.42 (d, J = 6.6 Hz, 1H), 4.71-4.59 (m, 2H), 4.57-4.51 (m, 1H), 4.46 (d, J = 4.2 Hz, 2H), 2.99-2.86 (m, 2H), |
| | | | | 2.43-2.32 (m, 1H), 2.18-2.07 (m, 1H), |
| | | | | 1.64-1.58 (m, 1H), 1.56 (d, J = 6.7 Hz, 3H), |
| | | | | 1.54-1.37 (m, 3H), 0.87-0.81 (m, 1H), |
| | | | | 0.71-0.67 (m, 3H). LC/MS (ESI) m/z: 590 |
| | | | | (M + H)⁺. RT (Method A): 1.92 min. |
| 382^{b} | 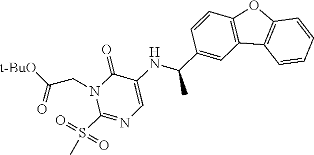 | 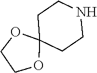 | 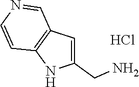 [SMILES: NCc1cc2cnccc2[nH]1 ; Cl] | ¹H NMR (400 MHz, DMSO-d₆) δ 11.31 (s, 1H), 8.79 (t, J = 5.5 Hz, 1H), 8.73 (s, 1H), 8.18 (s, 1H), 8.12-8.08 (m, 2H), 7.70- 7.63 (m, 2H), 7.60-7.56 (m, 1H), 7.51 (t, J = 7.8 Hz, 1H), 7.39 (t, J = 7.5 Hz, 1H), 7.32 (d, J = 5.7 Hz, 1H), 6.73 (s, 1H), 6.43 (s, 1H), 5.47 (d, J = 6.8 Hz, 1H), 4.73-4.62 (m, 2H), 4.57-4.51 (m, 1H), 4.46 (d, J = 5.6 Hz, 2H), 3.83-3.79 (m, 4H), 2.86-2.80 (m, 4H), 1.61-1.53 (m, 7H). LC/MS (ESI) m/z: 634 (M + H)⁺. RT (Method A): 1.66 min. |
| 384^{b} | 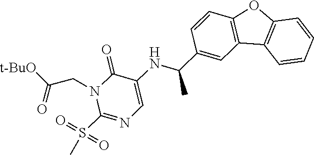 | 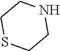 | 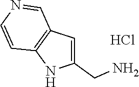 [SMILES: NCc1cc2cnccc2[nH]1 ; Cl] | ¹H NMR (400 MHz, CD₃OD) δ 8.76 (s, 1H), 8.13 (d, J = 6.0 Hz, 1H), 8.03 (s, 1H), 7.98 (d, J = 7.6 Hz, 1H), 7.56-7.49 (m, 3H), 7.48- 7.44 (m, 2H), 7.33 (t, J = 7.5 Hz, 1H), 6.73 (s, 1H), 6.64 (s, 1H), 4.83 (s, 2H), 4.63- 4.61 (m, 2H), 4.59-4.56 (m, 1H), 3.10- 3.04 (m, 4H), 2.56-2.51 (m, 4H), 1.63 (d, J = 6.7 Hz, 3H). LC/MS (ESI) (m/z): 594 (M + H)⁺. RT (Method A): 1.74 min. |
| 385^{b} | 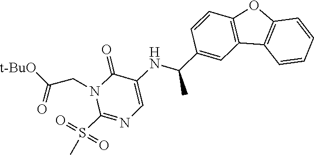 | 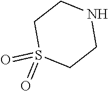 | 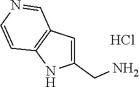 [SMILES: NCc1cc2cnccc2[nH]1 ; Cl] | ¹H NMR (400 MHz, DMSO-d₆) δ 11.32 (s, 1H), 8.84 (t, J = 5.5 Hz, 1H), 8.74 (s, 1H), 8.18 (d, J = 5.3 Hz, 1H), 8.13-8.08 (m, 2H), 7.68 (d, J = 8.2 Hz, 1H), 7.65 (d, J = 8.5 Hz, 1H), 7.61-7.57 (m, 1H), 7.53-7.49 (m, 1H), 7.39 (t, J = 7.5 Hz, 1H), 7.33 (d, J = 5.7 Hz, 1H), 6.76 (s, 1H), 6.46 (s, 1H), 5.65 (d, J = 6.9 Hz, 1H), 4.77-4.71 (m, 2H), 4.57 (t, J = 6.8 Hz, 1H), 4.48 (d, J = 5.6 Hz, 2H), 3.28-3.25 (m, 4H), 3.14-3.10 (m, 4H), 1.56 (d, J = 6.7 Hz, 3H). LC/MS (ESI) m/z: 626 (M + H)⁺. RT (Method A): 1.53 min. |
| 386^{b} | 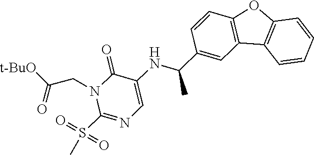 [SMILES: C[C@@H](Nc1cnc(S(C)(=O)=O)n(CC(=O)OC(C)(C)C)c1=O)c1ccc2oc3ccccc3c2c1] | 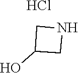 | 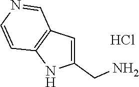 [SMILES: NCc1cc2cnccc2[nH]1 ; Cl] | ¹H NMR (400 MHz, DMSO-d₆) δ 11.38 (s, 1H), 8.80-8.72 (m, 2H), 8.16 (s, 1H), 8.11 (t, J = 6.4 Hz, 2H), 7.68 (d, J = 8.3 Hz, 1H), 7.63 (d, J = 8.4 Hz, 1H), 7.56 (d, J = 8.3 Hz, 1H), 7.51 (t, J = 7.5 Hz, 1H), 7.39 (t, J = 7.6 Hz, 1H), 7.32 (d, J = 5.7 Hz, 1H), 6.71 (s, 1H), 6.44 (s, 1H), 5.48 (s, 1H), 5.08 (d, J = 6.7 Hz, 1H), 4.58-4.49 (m, 2H), 4.47 (d, J = 5.7 Hz, 2H), 4.30 (s, 1H), 3.93 (t, J = 7.5 Hz, 2H), 3.67-3.61 (m, 2H), 2.00 (d, J = 7.4 |
| | | | | Hz, 1H), 1.54 (d, J = 6.7 Hz, 3H). LC/MS |
| | | | | (ESI) m/z: 564 (M + H)⁺. RT (Method A): |
| | | | | 1.37 min. |
| 387^{b} | 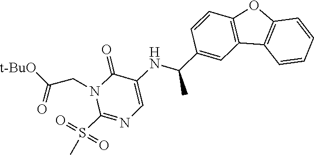 | 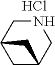 | 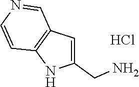 [SMILES: NCc1cc2cnccc2[nH]1 ; Cl] | ¹H NMR (400 MHz, CD₃OD) δ 8.70 (s, 1H), 8.09 (d, J = 5.6 Hz, 1H), 8.04 (s, 1H), 7.98 (d, J = 7.7 Hz, 1H), 7.57-7.50 (m, 3H), 7.46- 7.42 (m, 1H), 7.39-7.30 (m, 2H), 6.77 (s, 1H), 6.55 (s, 1H), 4.61-4.56 (m, 3H), 3.29- 3.23 (m, 4H), 2.22-2.18 (m, 2H), 2.00- 1.93 (m, 2H), 1.63 (d, J = 6.7 Hz, 3H), 1.45- 1.40 (m, 2H), 1.34-1.27 (m, 2H). LC/MS (ESI) m/z: 587 (M + H)⁺. RT (Method A): 1.84 min. |
| 388^{b} | 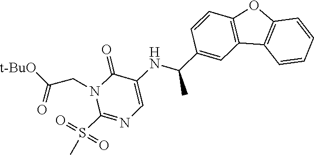 [SMILES: C[C@@H](Nc1cnc(S(C)(=O)=O)n(CC(=O)OC(C)(C)C)c1=O)c1ccc2oc3ccccc3c2c1] | 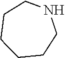 | 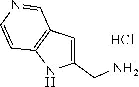 [SMILES: NCc1cc2cnccc2[nH]1 ; Cl] | ¹H NMR (400 MHz, DMSO-d₆) δ 11.30 (s, 1H), 8.77-8.69 (m, 2H), 8.16 (d, J = 1.1 Hz, 1H), 8.11-8.07 (m, 2H), 7.67 (d, J = 8.2 Hz, 1H), 7.63 (d, J = 8.5 Hz, 1H), 7.58- 7.54 (m, 1H), 7.49 (t, J = 7.7 Hz, 1H), 7.38 (t, J = 7.5 Hz, 1H), 7.31 (d, J = 5.7 Hz, 1H), 6.70 (s, 1H), 6.41 (s, 1H), 5.35 (d, J = 6.8 Hz, 1H), 4.65 (d, J = 3.6 Hz, 2H), 4.56-4.49 (m, 1H), 4.45 (d, J = 5.6 Hz, 2H), 2.99-2.93 (m, 4H), 1.54 (d, J = 6.7 Hz, 3H), 1.51-1.45 |
| | | | | (m, 8H). LC/MS (ESI) (m/z): 590 (M + H)⁺. |
| | | | | RT (Method A): 1.88 min. |
| 389^{b} | 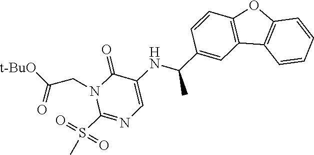 | 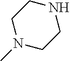 | 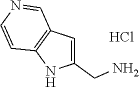 [SMILES: NCc1cc2cnccc2[nH]1 ; Cl] | ¹H NMR (400 MHz, DMSO-d₆) δ 11.37 (s, 1H), 8.81-8.73 (m, 2H), 8.17-8.10 (m, 3H), 7.69-7.48 (m, 4H), 7.39-7.32 (m, 2H), 6.74 (s, 1H), 6.43 (s, 1H), 5.47 (s, 1H), 4.66 (s, 2H), 4.57-4.51 (m, 1H), 4.46 (s, 2H), 2.80-2.70 (m, 4H), 2.24-2.08 (m, 4H), 2.07 (s, 3H), 1.56 (br, 3H). LC/MS (ESI) (m/z): 591 (M + H)⁺. RT (Method A): 1.06 min. |
| 397^{b} | 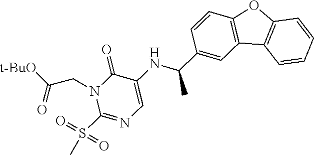 | 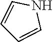 | 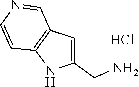 [SMILES: NCc1cc2cnccc2[nH]1 ; Cl] | ¹H NMR (400 MHz, DMSO-d₆) δ 11.36 (s, 1H), 8.83-8.69 (m, 2H), 8.16-8.03 (m, 3H), 7.71-7.60 (m, 3H), 7.54-7.49 (m, 1H), 7.40 (t, J = 7.1 Hz, 1H), 7.33 (d, J = 5.7 Hz, 1H), 6.98-6.88 (m, 2H), 6.87 (s, 1H), 6.38 (s, 1H), 6.17-6.09 (m, 2H), 6.07 (d, J = 7.3 Hz, 1H), 4.68 (t, J = 7.0 Hz, 1H), 4.47- 4.35 (m, 3H), 4.25 (d, J = 16.1 Hz, 1H), 1.61 (d, J = 6.7 Hz, 3H). LC/MS (ESI) m/z: 558 (M + H)⁺. RT (Method A): 1.41 min. |
| 398^{b} | 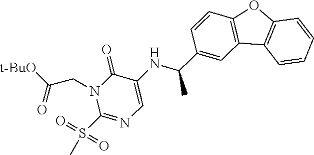 [SMILES: C[C@@H](Nc1cnc(S(C)(=O)=O)n(CC(=O)OC(C)(C)C)c1=O)c1ccc2oc3ccccc3c2c1] | 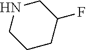 | 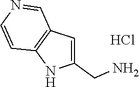 [SMILES: NCc1cc2cnccc2[nH]1 ; Cl] | ¹H NMR (400 MHz, DMSO-d₆) δ 11.34 (s, 1H), 8.80 (t, J = 5.4 Hz, 1H), 8.73 (s, 1H), 8.18 (s, 1H), 8.10 (t, J = 7.2 Hz, 2H), 7.71- 7.62 (m, 2H), 7.60-7.56 (m, 1H), 7.51 (t, J = 7.7 Hz, 1H), 7.39 (t, J = 7.5 Hz, 1H), 7.32 (d, J = 5.6 Hz, 1H), 6.74 (s, 1H), 6.44 (s, 1H), 5.50 (d, J = 6.8 Hz, 1H), 4.76-4.65 (m, 2H), 4.64-4.51 (m, 2H), 4.50-4.40 (m, 2H), 3.00-2.89 (m, 2H), 2.80-2.68 (m, 2H), 1.78-1.61 (m, 3H), 1.56 (d, J = |
| | | | | 6.7 Hz, 3H), 1.48-1.39 (m, 1H) . LC/MS |
| | | | | (ESI) m/z: 594 (M + H)⁺. RT (Method A): |
| | | | | 1.68 min. |
| 399^{c} | 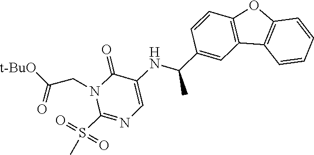 [SMILES: C[C@@H](Nc1cnc(S(C)(=O)=O)n(CC(=O)OC(C)(C)C)c1=O)c1ccc2oc3ccccc3c2c1] | 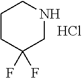 | 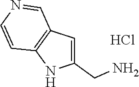 [SMILES: NCc1cc2cnccc2[nH]1 ; Cl] | ¹H NMR (400 MHz, DMSO-d₆) δ 11.35 (s, 1H), 8.83 (t, J = 5.7 Hz, 1H), 8.73 (s, 1H), 8.18 (d, J = 1.5 Hz, 1H), 8.10 (t, J = 7.2 Hz, 2H), 7.66 (dd, J = 15.3, 8.4 Hz, 2H), 7.58 (dd, J = 8.5, 1.7 Hz, 1H), 7.53-7.49 (m, 1H), 7.41-7.37 (m, 1H), 7.32 (d, J = 5.6 Hz, 1H), 6.75 (s, 1H), 6.43 (s, 1H), 5.58 (d, J = 6.9 Hz, 1H), 4.72-4.63 (m, 2H), 4.56 (t, J = 6.7 Hz, 1H), 4.46 (d, J = 5.6 Hz, 2H), 3.07 (t, J = 11.4 Hz, 2H), 2.84-2.77 (m, |
| | | | | 2H), 1.98-1.87 (m, 2H), 1.68-1.62 (m, |
| | | | | 2H), 1.56 (d, J = 6.7 Hz, 3H). LC/MS (ESI) |
| | | | | (m/z): 612 (M + H)⁺. RT (Method A): 1.77 |
| | | | | min. |
| 400^{b} | 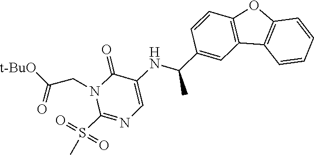 [SMILES: C[C@@H](Nc1cnc(S(C)(=O)=O)n(CC(=O)OC(C)(C)C)c1=O)c1ccc2oc3ccccc3c2c1] | 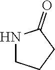 | 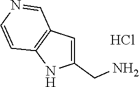 [SMILES: NCc1cc2cnccc2[nH]1 ; Cl] | ¹H NMR (400 MHz, DMSO-d₆) δ 12.36 (s, 1H), 9.10 (s, 1H), 8.98 (t, J = 5.5 Hz, 1H), 8.32 (d, J = 6.4 Hz, 1H), 8.09 (d, J = 7.5 Hz, 1H), 7.78 (d, J = 6.4 Hz, 1H), 7.70-7.62 (m, 2H), 7.58 (dd, J = 8.5, 1.6 Hz, 1H), 7.54- 7.49 (m, 1H), 7.42-7.36 (m, 1H), 6.82 (s, 1H), 6.78 (s, 1H), 5.95 (d, J = 7.1 Hz, 1H), 4.90-4.72 (m, 1H), 4.77-4.57 (m, 2H), 4.53 (d, J = 5.6 Hz, 2H), 3.51-3.40 (m, 2H), 2.31 (t, J = 8.0 Hz, 2H), 1.80-1.67 (m, 2H), |
| | | | | 1.59 (d, J = 6.7 Hz, 3H). LC/MS (ESI) m/z: |
| | | | | 557 (M + H)⁺. RT (Method A): 1.46 min. |
| 401^{b} | 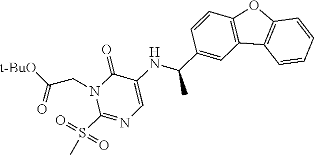 | 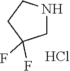 | 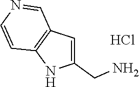 [SMILES: NCc1cc2cnccc2[nH]1 ; Cl] | ¹H NMR (400 MHz, DMSO-d₆) δ 11.36 (s, 1H), 8.83 (t, J = 5.3 Hz, 1H), 8.73 (s, 1H), 8.17 (s, 1H), 8.13-8.06 (m, 2H), 7.69- 7.62 (m, 2H), 7.58 (d, J = 8.4 Hz, 1H), 7.51 (t, J = 7.5 Hz, 1H), 7.39 (t, J = 7.4 Hz, 1H), 7.32 (d, J = 5.6 Hz, 1H), 6.73 (s, 1H), 6.44 (s, 1H), 5.45 (d, J = 6.8 Hz, 1H), 4.68-4.61 (m, 2H), 4.56-4.51 (m, 1H), 4.48 (s, 2H), 3.49 (t, J = 13.4 Hz, 2H), 3.31-3.26 (m, 2H), 2.31-2.22 (m, 2H), 1.56 (d, J = 6.6 Hz, 3H). LC/MS (ESI) (m/z): 598 (M + H)⁺. RT (Method A): 1.76 min. |
| 402^{c} | 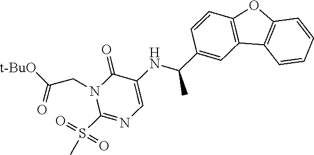 [SMILES: C[C@@H](Nc1cnc(S(C)(=O)=O)n(CC(=O)OC(C)(C)C)c1=O)c1ccc2oc3ccccc3c2c1] | 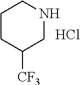 | 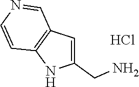 [SMILES: NCc1cc2cnccc2[nH]1 ; Cl] | ¹H -NMR (400 MHz, CD₃OD) δ 8.71 (s, 1H), 8.11 (d, J = 5.9 Hz, 1H), 8.04 (s, 1H), 7.98 (m, 1H), 7.57-7.50 (m, 3H), 7.48-7.44 (m, 1H), 7.40 (d, J = 5.7 Hz, 1H), 7.35-7.31(m, 1H), 6.74 (d, J = 2.7 Hz, 1H), 6.59 (s, 1H), 4.88-4.86 (m, 2H), 4.81-4.77 (m, 1H), 4.61 (s, 2H), 3.28-3.23 (m, 1H), 3.15-3.10 (m, 1H), 2.69-2.62 (m, 1H), 2.59- 2.47 (m, 1H), 2.42-2.34 (m, 1H), 1.91- 1.86 (m, 1H), 1.63 (d, J = 6.7 Hz, 3H), 1.58- |
| | | | | 1.51 (m, 1H), 1.38-1.26 (m, 2H). LC/MS |
| | | | | (ESI) (m/z): 644 (M + H)⁺. RT (Method A): |
| | | | | 1.95 min. |
| 415^{c} | 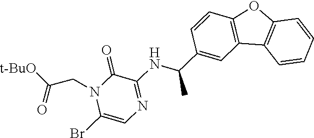 [SMILES: C[C@@H](Nc1ncc(Br)n(CC(=O)OC(C)(C)C)c1=O)c1ccc2oc3ccccc3c2c1] | 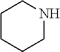 | 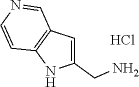 [SMILES: NCc1cc2cnccc2[nH]1 ; Cl] | ¹H NMR (400 MHz, DMSO-d₆) δ 11.31 (s, 1H), 8.75-8.69 (m, 2H), 8.15 (s, 1H), 8.13- 8.09 (m, 2H), 7.67 (d, J = 8.2 Hz, 1H), 7.63- 7.56 (m, 2H), 7.53-7.48 (m, 1H), 7.39 (t, J = 7.5 Hz, 1H), 7.32 (d, J = 5.7 Hz, 1H), 7.24 (d, J = 8.1 Hz, 1H), 6.57 (s, 1H), 6.42 (s, 1H), 5.27-5.18 (m, 1H), 4.71-4.62 (m, 2H), 4.45 (d, J = 5.6 Hz, 2H), 2.95-2.83 (m, 2H), 2.48-2.38 (m, 2H), 1.64-1.55 (m, |
| | | | | 4H), 1.51-1.33 (m, 4H), 1.18-1.05 (m, |
| | | | | 1H). LC/MS (ESI) m/z: 576 (M + H)⁺. RT |
| | | | | (Method A): 1.83 min. |
| 417^{c} | 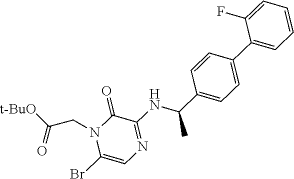 | 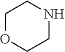 | 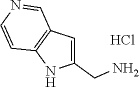 [SMILES: NCc1cc2cnccc2[nH]1 ; Cl] | ¹H NMR (400 MHz, CD₃OD) δ 8.72 (s, 1H), 8.11 (d, J = 5.9 Hz, 1H), 7.49-7.44 (m, 5H), 7.41 (d, J = 6.7 Hz, 1H), 7.36-7.31 (m, 1H), 7.22 (t, J = 7.0 Hz, 1H), 7.18-7.13 (m, 1H), 6.68 (s, 1H), 6.58 (s, 1H), 5.16-5.10 (m, 1H), 4.86 (s, 2H), 4.59 (s, 2H), 3.70-3.60 (m, 2H), 3.50-3.40 (m, 2H), 2.84-2.77 (m, 4H), 1.60 (d, J = 6.9 Hz, 3H). LC/MS (ESI) m/z: 582 (M + H)⁺. RT (Method A: 1.56 min. |
| 419^{d} | 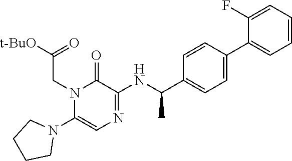 | | 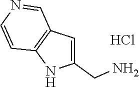 [SMILES: NCc1cc2cnccc2[nH]1 ; Cl] | ¹H NMR (400 MHz, DMSO-d₆) δ 11.31 (s, 1H), 8.73 (s, 1H), 8.68 (t, J = 5.7 Hz, 1H), 8.11 (d, J = 5.6 Hz, 1H), 7.52-7.46 (m, 5H), 7.41-7.38 (m, 1H), 7.33-7.31 (m, 1H), 7.30-7.26 (m, 2H), 7.14 (d, J = 8.1 Hz, 1H), 6.66 (s, 1H), 6.41 (s, 1H), 5.12-5.04 (m, 1H), 4.65 (s, 2H), 4.45 (d, J = 5.5 Hz, 2H), 2.89-2.83 (m, 4H), 1.74-1.68 (m, 4H), 1.52 (d, J = 7.0 Hz, 3H). LC/MS (ESI) m/z: 566 (M + H)⁺. RT (Method A): 1.76 min. |
| 421^{c} | 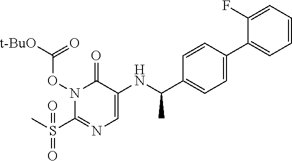 | 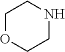 | 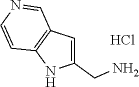 [SMILES: NCc1cc2cnccc2[nH]1 ; Cl] | ¹H NMR (400 MHz, CD₃OD) δ 8.71 (s, 1H), 8.11 (d, J = 5.8 Hz, 1H), 7.52-7.47 (m, 2H), 7.44 (t, J = 7.2 Hz, 3H), 7.39-7.32 (m, 2H), 7.26-7.16 (m, 2H), 6.71 (s, 1H), 6.58 (s, 1H), 4.86 (s, 2H), 4.61 (s, 2H), 4.46 (d, J = 6.7 Hz, 1H), 3.67-3.53 (m, 4H), 2.92-2.84 (m, 4H), 1.58 (d, J = 6.7 Hz, 3H). LC/MS (ESI) m/z: 582 (M + H)⁺. RT (Method A): 1.57 min. |
| 422^{c} | 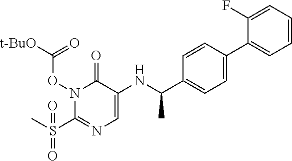 | 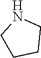 | 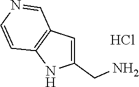 [SMILES: NCc1cc2cnccc2[nH]1 ; Cl] | ¹H NMR (400 MHz, DMSO-d₆) δ 11.33 (s, 1H), 8.82- 8.68 (m, 2H), 8.11 (d, J = 5.6 Hz, 1H), 7.55-7.46 (m, 5H), 7.40 (dd, J = 12.9, 6.2 Hz, 1H), 7.34-7.26 (m, 3H), 6.65 (s, 1H), 6.43 (s, 1H), 5.15 (d, J = 6.9 Hz, 1H), 4.69-4.57 (m, 2H), 4.47 (d, J = 5.4 Hz, 2H), 4.43-4.35 (m, 1H), 3.20-3.10 (m, 4H), 1.77- 1.65 (m, 4H), 1.49 (d, J = 6.7 Hz, 3H). LC/MS (ESI) m/z: 566 (M + H)⁺. RT (Method A): 1.66 min. |
| 423^{c} | 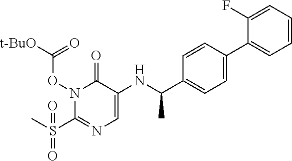 | 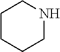 | 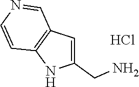 [SMILES: NCc1cc2cnccc2[nH]1 ; Cl] | ¹H NMR (400 MHz, CD₃OD) δ 8.70 (s, 1H), 8.09 (d, J = 5.9 Hz, 1H), 7.49-7.45 (m, 4H), 7.44-7.42 (m, 1H), 7.39-7.30 (m, 2H), 7.24-7.20 (m, 1H), 7.18-7.13 (m, 1H), 6.61 (s, 1H), 6.56 (s, 1H), 5.14-5.07 (m, 1H), 4.83 (s, 2H), 4.59 (s, 2H), 3.01-2.93 (m, 2H), 2.60-2.50 (m, 2H), 1.70-1.63 (m, 1H), 1.59 (d, J = 6.9 Hz, 3H), 1.56-1.42 (m, 4H), 1.30-1.20 (m, 1H) . LC/MS (ESI) m/z: 580 (M + H)⁺. RT (Method A): 1.87 min. |
| 424^{c} | 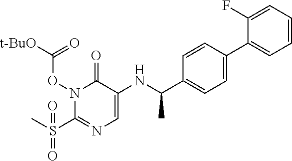 [SMILES: C[C@@H](Nc1cnc(S(C)(=O)=O)n(OC(=O)OC(C)(C)C)c1=O)c1ccc(-c2ccccc2F)cc1] | 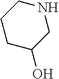 | 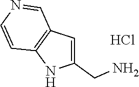 [SMILES: NCc1cc2cnccc2[nH]1 ; Cl] | ¹H NMR (400 MHz, DMSO-d₆) δ 11.32 (s, 1H), 8.73 (s, 1H), 8.70 (t, J = 5.6 Hz, 1H), 8.16 (s, 1H), 8.11 (s, 1H), 8.10 (s, 1H), 7.67 (d, J = 8.2 Hz, 1H), 7.62-7.58 (m, 2H), 7.50 (t, J = 7.2 Hz, 1H), 7.39 (t, J = 7.4 Hz, 1H), 7.32 (d, J = 5.6 Hz, 1H), 7.27-7.23 (m, 1H), 6.59 (s, 1H), 6.42 (s, 1H), 5.25-5.19 (m, 1H), 4.76 (dd, J = 12.3, 4.7 Hz, 1H), 4.67- 4.60 (m, 2H), 4.48-4.43 (m, 2H), 3.50- 3.37 (m, 1H), 3.00-2.82 (m, 2H), 2.36- |
| | | | | 2.22 (m, 2H), 1.57 (d, J = 6.9 Hz, 3H), 1.49- |
| | | | | 1.35 (m, 2H), 1.26-1.01 (m, 2H). LC/MS |
| | | | | (ESI) m/z: 592 (M + H)⁺. RT (Method A): |
| | | | | 1.51 min. |
| 428^{c} | 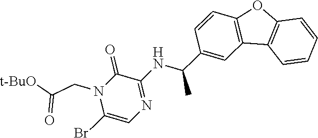 | 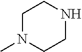 | 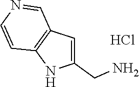 [SMILES: NCc1cc2cnccc2[nH]1 ; Cl] | ¹H NMR (400 MHz, CD₃OD) δ 8.69 (s, 1H), 8.09 (d, J = 5.8 Hz, 1H), 8.04 (s, 1H), 7.97 (d, J = 7.6 Hz, 1H), 7.55-7.49 (m, 3H), 7.43 (d, J = 7.6 Hz, 1H), 7.37-7.30 (m, 2H), 6.66 (s, 1H), 6.55 (s, 1H), 5.27-5.22 (m, 1H), 4.80 (s, 2H), 4.57 (s, 2H), 2.89-2.72 (m, 4H), 2.59-2.52 (m, 2H), 2.11 (s, 3H), 2.01- 1.92 (m, 2H), 1.64 (d, J = 6.9 Hz, 3H). LC/MS (ESI) m/z: 591 (M + H)⁺. RT (Method A): 0.99 min. |
| 440^{c} | 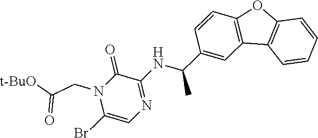 [SMILES: C[C@@H](Nc1ncc(Br)n(CC(=O)OC(C)(C)C)c1=O)c1ccc2oc3ccccc3c2c1] | 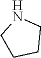 | 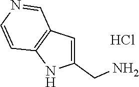 [SMILES: NCc1cc2cnccc2[nH]1 ; Cl] | ¹H NMR (400 MHz, DMSO-d₆) δ 11.29 (s, 1H), 8.72 (s, 1H), 8.68 (t, J = 5.6 Hz, 1H), 8.15 (s, 1H), 8.12- 8.07 (m, 2H), 7.67 (d, J = 8.2 Hz, 1H), 7.63-7.55 (m, 2H), 7.53- 7.46 (m, 1H), 7.39 (t, J = 7.5 Hz, 1H), 7.32 (d, J = 5.6 Hz, 1H), 7.16 (d, J = 8.0 Hz, 1H), 6.65 (s, 1H), 6.41 (s, 1H), 5.25-5.13 (m, 1H), 4.69-4.58 (m, 2H), 4.44 (d, J = 5.7 Hz, 2H), 2.90-2.80 (m, 4H), 1.73-1.66 (m, |
| | | | | 4H), 1.57 (d, J = 7.0 Hz, 3H). LC/MS (ESI) |
| | | | | m/z: 562 (M + H)⁺. RT (Method A): 1.77 min. |
| 470^{c} | 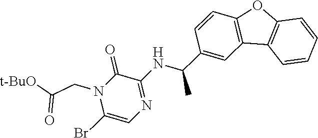 [SMILES: C[C@@H](Nc1ncc(Br)n(CC(=O)OC(C)(C)C)c1=O)c1ccc2oc3ccccc3c2c1] | 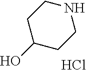 | 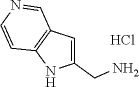 [SMILES: NCc1cc2cnccc2[nH]1 ; Cl] | ¹H NMR (400 MHz, DMSO-d₆) δ 11.37- 11.30 (m, 1H), 8.73 (s, 2H), 8.15 (s, 1H), 8.11 (s, 1H), 8.10 (s, 1H), 7.67 (d, J = 8.2 Hz, 1H), 7.62-7.57 (m, 2H), 7.52-7.48 (m, 1H), 7.38 (d, J = 7.4 Hz, 1H), 7.32 (d, J = 5.6 Hz, 1H), 7.23 (d, J = 8.0 Hz, 1H), 6.58 (s, 1H), 6.42 (s, 1H), 5.25-5.20 (m, 1H), 4.68-4.65 (m, 2H), 4.47-4.44 (m, 2H), 3.42-3.38 (m, 1H), 2.92-2.82 (m, 2H), 2.65- |
| | | | | 2.51 (m, 2H), 1.81-1.60 (m, 2H), 1.57 (d, |
| | | | | J = 7.0 Hz, 3H), 1.42-1.28 (m, 2H). LC/MS |
| | | | | (ESI) m/z: 592 (M + H)⁺. RT (Method A): |
| | | | | 1.64 min. |

^{a}Step 1 was performed in the presence of DIPEA in 1,4-dioxane.
^{b}Step 1 was performed in the presence of CsF in NMP.
^{c}Step 1 was performed in the presence of CsF in DMSO.
^{d}Steps 2 and 3 only. Step 2 was performed in MeOH, 1,4-dioxane, and H₂O.

### Scheme 116. Synthesis of (R)—N-((1H-pyrrolo[3,2-c]pyridine-2-yl)methyl)-2-(5-((1-(dibenzo[b,d]furan-2-yl)ethyl)amino)-2-(4-(3-fluorooxetan-3-yl)phenyl)-6-oxopyrimidin-1(6H)-yl)acetamide (Compound 372)

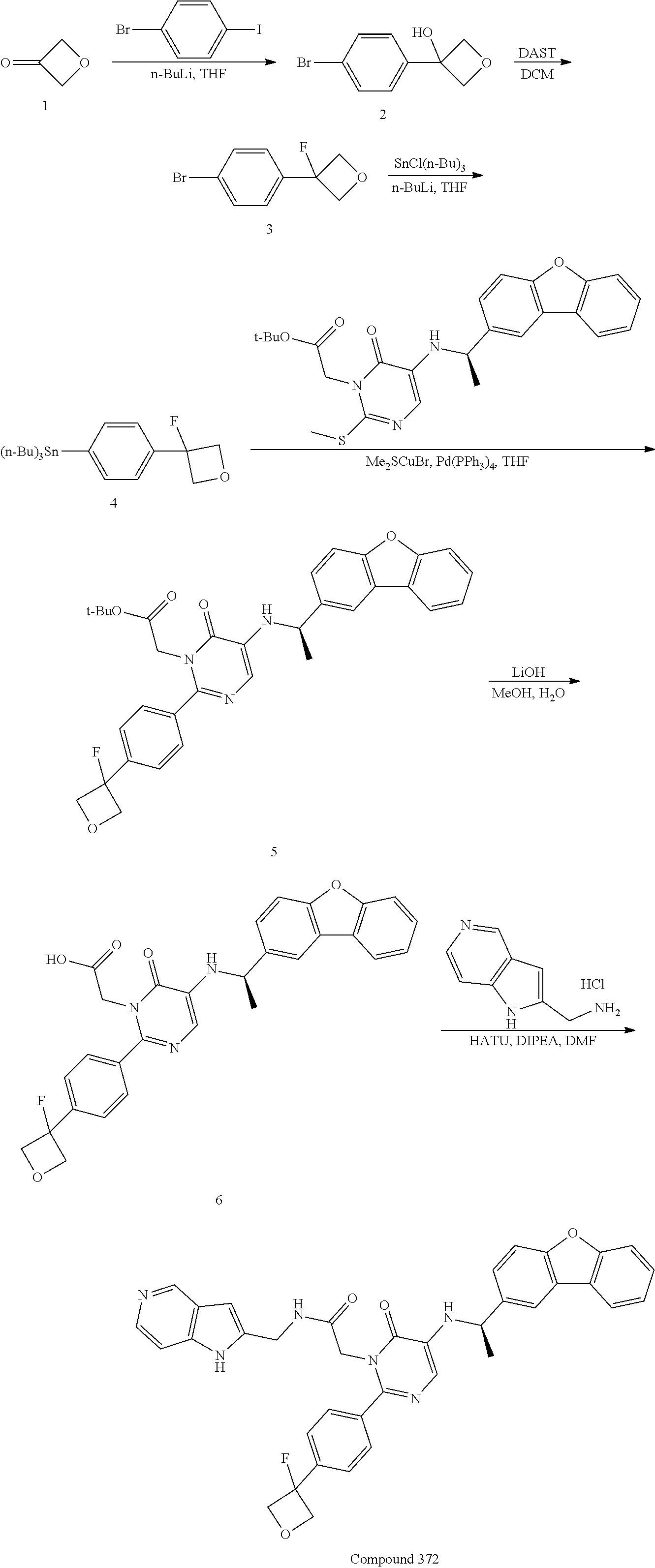

### Step 1: 3-(4-Bromophenyl)oxetan-3-ol (2)

To a solution of 1-bromo-4-iodobenzene (10.0 g, 35.5 mmol) in THF (100 mL) was added n-BuLi (17.0 mL, 42.5 mmol, 2.5 M in THF) dropwise under N₂ atmosphere at −70° C. and the mixture was stirred at this temperature for 0.5 hour. Oxetan-3-one (2.56 g, 35.5 mmol) was added to the above mixture at −70° C. and the resulting mixture was stirred at −70° C. for 2 hours. The mixture was quenched with ice-water and extracted with EtOAc twice. The combined organic layers were washed with brine, dried over anhydrous Na₂SO₄, filtered, and concentrated under reduced pressure to dryness. The residue was purified by flash chromatography (silica gel, 0-8% EtOAc in PE) to give the title compound (1.27 g, yield 15.6%) as a white solid. ¹H NMR (400 MHz, DMSO-d₆) δ 7.64-7.52 (m, 4H), 6.46 (s, 1H), 4.78 (d, 2H), 4.64 (d, J=6.6 Hz, 2H).

### Step 2: 3-(4-Bromophenyl)-3-fluorooxetane (3)

To a solution of 3-(4-bromophenyl)oxetan-3-ol (1.00 g, 4.38 mmol) in DCM (10 mL) was added DAST (2.82 g, 17.5 mmol) dropwise under N₂ atmosphere at 0° C. The mixture was stirred at 25° C. for 16 hours. The mixture was quenched with ice-water and extracted with EtOAc twice. The combined organic layers were dried over anhydrous Na₂SO₄, filtered, and concentrated under reduced pressure to dryness. The residue was purified by flash chromatography (silica gel, 0-10% EtOAc in PE) to give the title compound (286 mg, yield 28.6%) as a white solid. ¹H NMR (400 MHz, DMSO-d₆) δ 7.66 (s, 1H), 7.64 (s, 1H), 7.47 (s, 1H), 7.45 (s, 1H), 5.20 (d, J=7.7 Hz, 2H), 5.06-4.99 (m, 2H).

### Step 3: Tributyl(4-(3-fluorooxetan-3-yl)phenyl)stannane (4)

To a solution of 3-(4-bromophenyl)-3-fluorooxetane (286 mg, 1.24 mmol) in THF (5 mL) was added n-BuLi (0.74 mL, 1.86 mmol, 2.5 M in THF) dropwise under N₂ atmosphere at −70° C. and the mixture was stirred at this temperature for 0.5 hour. SnCl(n-Bu)₃ (807 mg, 2.48 mmol) was added to the above mixture at −70° C. and the resulting mixture was stirred at −70° C. for 2 hours. The mixture was quenched with ice-water and extracted with EtOAc. The combined organic layers were washed with brine, dried over anhydrous Na₂SO₄, filtered, and concentrated under reduced pressure to dryness. The residue was purified by flash chromatography (silica gel, 0-5% EtOAc in PE) to give the title compound (350 mg, yield 63.8%) as a yellow oil, which was used directly in the next step without further purification.

### Step 4: Tert-butyl (R)-2-(5-((1-(dibenzo[b,d]furan-2-yl)ethyl)amino)-2-(4-(3-fluorooxetan-3-yl)phenyl)-6-oxopyrimidin-1(6H)-yl)acetate (5)

To a mixture of tributyl(4-(3-fluorooxetan-3-yl)phenyl)stannane (350 mg, 0.79 mmol) and tert-butyl (R)-2-(5-((1-(dibenzo[b,d]furan-2-yl)ethyl)amino)-2-(methylthio)-6-oxopyrimidin-1(6H)-yl)acetate (441 mg, 0.95 mmol) in THF (10 mL) was added Copper(I) bromide-dimethyl sulfide complex (244 mg, 1.18 mmol) and Pd(PPh₃)₄ (183 mg, 0.16 mmol) under N₂ atmosphere, the reaction mixture was degassed under N₂ atmosphere for three times and stirred at 80° C. overnight. The mixture was diluted with water and extracted with EtOAc twice. The combined organic layers were washed with water and brine, dried over anhydrous Na₂SO₄, filtered, and concentrated under reduced pressure to dryness. The residue was purified by flash chromatography (silica gel, 0-45% EtOAc in PE) to give the title compound (250 mg, yield 55.5%) as a yellow oil. LC/MS (ESI) m/z: 570 (M+H)⁺.

### Step 5: (R)2-(5-((1-(dibenzo[b,d]furan-2-yl)ethyl)amino)-2-(4-(3-fluorooxetan-3-yl)phenyl)-6-oxopyrimidin-1(6H)-yl)acetic acid (6)

To a solution of tert-butyl (R)-2-(5-((1-(dibenzo[b,d]furan-2-yl)ethyl)amino)-2-(4-(3-fluorooxetan-3-yl)phenyl)-6-oxopyrimidin-1(6H)-yl)acetate (150 mg, 0.26 mmol) in MeOH (3 mL) and water (1 mL) was added LiOH (72 mg, 1.71 mmol) and the mixture was stirred at 45° C. for 6 hours. The mixture was concentrated under reduced pressure to dryness to give the title compound (136 mg, crude) as a brown solid, which was used directly in the next step without further purification. LC/MS (ESI) m/z: 514 (M+H)⁺.

### Step 6: (R)N-((1H-pyrrolo[3,2-c]pyridine-2-yl)methyl)-2-(5-((1-(dibenzo[b,d]furan-2-yl)ethyl)amino)-2-(4-(3-fluorooxetan-3-yl)phenyl)-6-oxopyrimidin-1(6H)-yl)acetamide (Compound 372)

To a mixture of (R)-2-(5-((1-(dibenzo[b,d]furan-2-yl)ethyl)amino)-2-(4-(3-fluorooxetan-3-yl)phenyl)-6-oxopyrimidin-1(6H)-yl)acetic acid (120 mg, 0.23 mmol) and (1H-pyrrolo[3,2-c]pyridine-2-yl) methanamine hydrochloride (35.0 mg, 0.24 mmol) in DMF (3 mL) was added DIPEA (181 mg, 1.40 mmol) and HATU (106 mg, 0.27 mmol) and the reaction mixture was stirred at room temperature for 1 hour. The mixture was diluted with EtOAc, washed with water, dried over anhydrous Na₂SO₄, filtered, and concentrated under reduced pressure to dryness. The residue was purified by flash chromatography (silica gel, 0-10% MeOH in DCM) and further purified by prep-HPLC to give Compound 372 (4.9 mg, yield 3.3%) as a white solid. ¹H NMR (400 MHz, DMSO-d₆) δ 11.30 (s, 1H), 8.80-8.49 (m, 2H), 8.22 (d, J=1.1 Hz, 1H), 8.09 (d, J=7.4 Hz, 2H), 7.69-7.64 (m, 2H), 7.63-7.60 (m, 1H), 7.53-7.47 (m, 5H), 7.38 (t, J=7.3 Hz, 1H), 7.33 (d, J=4.8 Hz, 1H), 6.99 (s, 1H), 6.34 (s, 1H), 5.96 (d, J=7.1 Hz, 1H), 4.94 (d, J=8.6 Hz, 1H), 4.90-4.84 (m, 2H), 4.79 (m, 1H), 4.71-4.62 (m, 1H), 4.56-4.49 (m, 2H), 4.45-4.38 (m, 3H), 1.60 (d, J=6.7 Hz, 3H). LC/MS (ESI) m/z: 643 (M+H)⁺. RT (Method A): 1.71 min.

### Scheme 117. Synthesis of (S*)—N-((1H-pyrrolo[3,2-c]pyridine-2-yl)methyl)-2-(5-((1-(dibenzo[b,d]furan-2-yl)-2-fluoroethyl)amino)-2-(2-fluorophenyl)-6-oxopyrimidin-1(6H)-yl)acetamide (Compound 375)

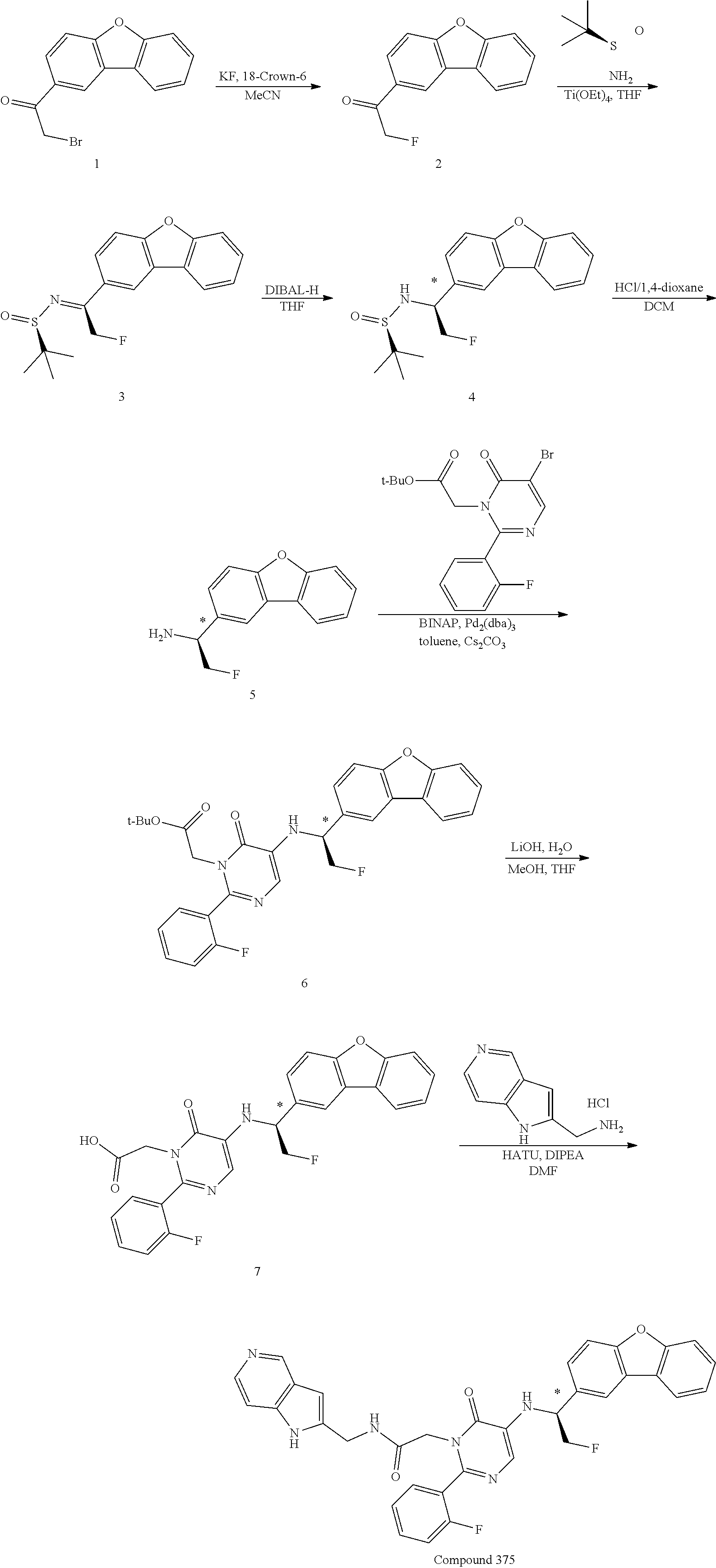

### Step 1: 1-(Dibenzo[b,d]furan-2-yl)-2-fluoroethan-1-one (2)

To a solution of 2-bromo-1-(dibenzo[b,d]furan-2-yl)ethan-1-one (980 mg, 3.40 mmol) in MeCN (20 mL) was added KF (439 mg, 7.56 mmol) and 18-Crown-6 (179 mg, 0.68 mmol) and the mixture was stirred at 85° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-4% EtOAc in PE) to give the title compound (535 mg, yield 69.2%) as a white solid. ¹H NMR (400 MHz, CDCl₃) δ 8.57 (s, 1H), 8.07-8.00 (m, 2H), 7.67-7.60 (m, 2H), 7.54 (t, J=7.7 Hz, 1H), 7.42 (t, J=7.4 Hz, 1H), 5.69 (m, 1H), 5.58 (m, 1H).

### Step 2: (R,Z)—N-(1-(dibenzo[b,d]furan-2-yl)-2-fluoroethylidene)-2-methylpropane-2-sulfinamide (3)

To a mixture of 1-(dibenzo[b,d]furan-2-yl)-2-fluoroethan-1-one (520 mg, 2.28 mmol) and (R)-2-methylpropane-2-sulfinamide (829 mg, 6.84 mmol) in THF (8 mL) was added Ti(OEt)₄ (1.56 g, 6.84 mmol) and the mixture was stirred at 70° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-9% EtOAc in PE) to give the title compound (360 mg, yield 47.7%) as a yellow solid. LC/MS (ESI) (m/z): 332 (M+H)⁺.

### Step 3: (R)—N—((S*)-1-(dibenzo[b,d]Furan-2-yl)-2-fluoroethyl)-2-methylpropane-2-sulfinamide (4)

To a solution of (R,Z)—N-(1-(dibenzo[b,d]furan-2-yl)-2-fluoroethylidene)-2-methylpropane-2-sulfinamide (340 mg, 1.03 mmol) in THF (5 mL) was added DIBAL-H (1.7 mL, 2.58 mmol, 1.5 M in THF) dropwise at −78° C. under N₂ atmosphere and the mixture was stirred under N₂ atmosphere at −78° C. for 15 minutes. The mixture was quenched with saturated aq. Potassium sodium tartrate solution at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give the title compound (220 mg, yield 64.3%) as a yellow solid. LC/MS (ESI) m/z: 334 (M+H)⁺.

### Step 4: (S*)-1-(dibenzo[b,d]furan-2-yl)-2-fluoroethan-1-amine (5)

To a solution of (R)—N—((S)-1-(dibenzo[b,d]furan-2-yl)-2-fluoroethyl)-2-methylpropane-2-sulfinamide (220 mg, 0.66 mmol) in DCM (1 mL) was added HCl/1,4-dioxane (2 mL, 4M) and the reaction mixture was stirred under N₂ atmosphere at room temperature for 1 hour. The reaction mixture was concentrated under reduced pressure to dryness to give the title compound (150 mg, yield 99.3%) as a white solid, which was used directly in the next step without further purification. LC/MS (ESI) m/z: 230 (M+H)⁺.

### Step 5: Tert-butyl (S*)-2-(5-((1-(dibenzo[b,d]furan-2-yl)-2-fluoroethyl)amino)-2-(2-fluorophenyl)-6-oxopyrimidin-1(6H)-yl)acetate (6)

To a mixture of (S)-1-(dibenzo[b,d]furan-2-yl)-2-fluoroethan-1-amine (140 mg, 0.61 mmol) and tert-butyl 2-(5-bromo-2-(2-fluorophenyl)-6-oxopyrimidin-1(6H)-yl)acetate (234 mg, 0.61 mmol) in toluene (5 mL) was added Cs₂CO₃ (597 mg, 1.83 mmol), BINAP (38 mg, 0.06 mmol) and Pd₂(dba)₃ (56 mg, 0.06 mmol) under N₂ atmosphere, the mixture was degassed under N₂ atmosphere for three times and stirred at 100° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-15% EtOAc in PE) to give the title compound (200 mg, yield 61.7%) as a yellow solid. LC/MS (ESI) m/z: 531 (M+H)⁺.

### Step 6: (S*)-2-(5-((1-(dibenzo[b,d]furan-2-yl)-2-fluoroethyl)amino)-2-(2-fluorophenyl)-6-oxopyrimidin-1(6H)-yl)acetic acid (7)

To a solution of tert-butyl (S)-2-(5-((1-(dibenzo[b,d]furan-2-yl)-2-fluoroethyl)amino)-2-(2-fluorophenyl)-6-oxopyrimidin-1(6H)-yl)acetate (200 mg, 0.38 mmol) in MeOH (2 mL), THF (2 mL) and water (2 mL) was added LiOH (27 mg, 1.14 mmol). The reaction mixture was stirred at 50° C. overnight. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (150 mg, yield 84.3%) as a yellow solid, which was used directly in the next step without further purification. LC/MS (ESI) m/z: 476 (M+H)⁺.

### Step 7: (S*)—N-((1H-pyrrolo[3,2-c]pyridine-2-yl)methyl)-2-(5-((1-(dibenzo[b,d]furan-2-yl)-2-fluoroethyl)amino)-2-(2-fluorophenyl)-6-oxopyrimidin-1(6H)-yl)acetamide (Compound 375)

To a mixture of (S*)-2-(5-((1-(dibenzo[b,d]furan-2-yl)-2-fluoroethyl)amino)-2-(2-fluorophenyl)-6-oxopyrimidin-1(6H)-yl)acetic acid (100 mg, 0.21 mmol) and (1H-pyrrolo[3,2-c]pyridine-2-yl)methanamine hydrochloride (58 mg, 0.31 mmol) in DMF (3 mL) was added DIPEA (163 mg, 1.26 mmol) and HATU (96 mg, 0.25 mmol) and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 375 (15 mg, yield 11.8%) as a white solid. ¹H NMR (400 MHz, DMSO-d₆) δ 11.27 (s, 1H), 8.73 (s, 1H), 8.60 (t, J=5.5 Hz, 1H), 8.31 (s, 1H), 8.12-8.08 (m, 2H), 7.71-7.69 (m, 3H), 7.55-7.53 (m, 1H), 7.51-7.48 (m, 1H), 7.43-7.39 (m, 1H), 7.35 (d, J=7.2 Hz, 1H), 7.31-7.26 (m, 2H), 7.13 (m, 2H), 6.37 (d, J=7.6 Hz, 1H), 6.22 (s, 1H), 5.04-4.95 (m, 2H), 4.88-4.74 (m, 2H), 4.70-4.62 (m, 1H), 4.33 (d, J=5.5 Hz, 2H). LC/MS (ESI) (m/z): 605 (M+H)⁺. RT (Method A): 1.64 min.

### Scheme 118. Synthesis of (R)—N-((1H-pyrrolo[3,2-c]pyridine-2-yl)methyl)-2-(5-((1-(dibenzo[b,d]furan-2-yl)ethyl)amino)-6-oxo-2-(piperazin-1-yl)pyrimidin-1(6H)-yl)acetamide (Compound 376)

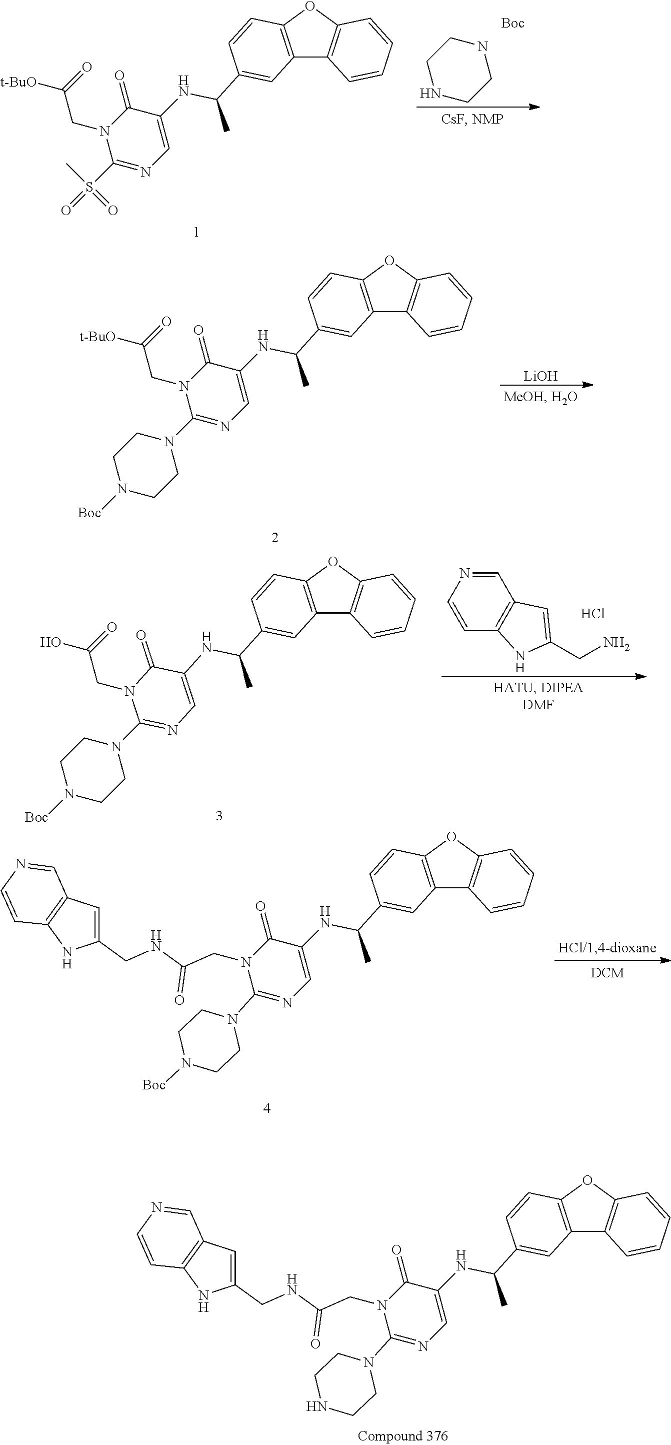

### Step 1: Tert-butyl (R)-4-(1-(2-(tert-butoxy)-2-oxoethyl)-5-((1-(dibenzo[b,d]furan-2-yl)ethyl)amino)-6-oxo-1,6-dihydropyrimidin-2-yl)piperazine-1-carboxylate (2)

To a mixture of tert-butyl (R)-2-(5-((1-(dibenzo[b,d]furan-2-yl)ethyl)amino)-2-(methylsulfonyl)-6-oxopyrimidin-1(6H)-yl)acetate (100 mg, 0.20 mmol) and tert-butyl piperazine-1-carboxylate (187 mg, 1.00 mmol) in NMP (2 mL) was added CsF (92 mg, 0.61 mmol) and the reaction mixture was stirred at 100° C. for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-22% EtOAc in PE) to give the title compound (60 mg, yield 49.4%) as a white solid. LC/MS (ESI) m/z: 604 (M+H)⁺.

### Step 2: (R)-2-(2-(4-(tert-butoxycarbonyl)piperazin-1-yl)-5-((1-(dibenzo[b,d]furan-2-yl)ethyl)amino)-6-oxopyrimidin-1(6H)-yl)acetic acid (3)

To a solution of tert-butyl (R)-4-(1-(2-(tert-butoxy)-2-oxoethyl)-5-((1-(dibenzo[b,d]furan-2-yl)ethyl)amino)-6-oxo-1,6-dihydropyrimidin-2-yl)piperazine-1-carboxylate (60 mg, 0.099 mmol) in MeOH/water (2 mL, v/v=4/1) was added LiOH·H₂O (8 mg, 0.19 mmol) and the reaction mixture was stirred at 60° C. overnight. The mixture was acidified with 1 N aq. HCl to pH˜3 and extracted with EtOAc twice.

The combined organic layers were dried over anhydrous Na₂SO₄, filtered, and concentrated under reduced pressure to give the title compound (54 mg, yield 99.2%) as a white solid, which was used directly in the next step without further purification. LC/MS (ESI) m/z: 548 (M+H)⁺.

### Step 3: Tert-butyl (R)-4-(1-(2-(((1H-pyrrolo[3,2-c]pyridine-2-yl)methyl)amino)-2-oxoethyl)-5-((1-(dibenzo[b,d]furan-2-yl)ethyl)amino)-6-oxo-1,6-dihydropyrimidin-2-yl)piperazine-1-carboxylate (4)

To a mixture of (R)-2-(2-(4-(tert-butoxycarbonyl)piperazin-1-yl)-5-((1-(dibenzo[b,d]furan-2-yl)ethyl)amino)-6-oxopyrimidin-1(6H)-yl)acetic acid (54 mg, 0.098 mmol) and (1H-pyrrolo[3,2-c]pyridin-2-yl)methanamine hydrochloride (22 mg, 0.12 mmol) in DMF (2 mL) was added DIPEA (65 mg, 0.50 mmol) and HATU (46 mg, 0.12 mmol) and the reaction mixture was stirred at room temperature for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-20% MeOH in DCM) to give the title compound (53 mg, yield 77.6%) as a yellow solid. LC/MS (ESI) m/z: 677 (M+H)⁺.

### Step 4: (R)—N-((1H-pyrrolo[3,2-c]pyridine-2-yl)methyl)-2-(5-((1-(dibenzo[b,d]furan-2-yl)ethyl)amino)-6-oxo-2-(piperazin-1-yl)pyrimidin-1(6H)-yl)acetamide (Compound 376)

To a solution of tert-butyl (R)-4-(1-(2-(((1H-pyrrolo[3,2-c]pyridine-2-yl)methyl)amino)-2-oxoethyl)-5-((1-(dibenzo[b,d]furan-2-yl)ethyl)amino)-6-oxo-1,6-dihydropyrimidin-2-yl)piperazine-1-carboxylate (53 mg, 0.078 mmol) in DCM (3 mL) was added HCl/1,4-dioxane (1 mL, 4M) and the reaction mixture was stirred at room temperature for 1 hour. The mixture was concentrated under reduced pressure to dryness. The residue was purified by prep-HPLC to give the title compound (20.4 mg, yield 45.1%) as a white solid. ¹H NMR (400 MHz, DMSO-d₆) δ 11.31 (s, 1H), 8.78 (s, 1H), 8.74 (s, 1H), 8.18 (s, 1H), 8.10 (t, J=5.8 Hz, 2H), 7.68 (d, J=8.2 Hz, 1H), 7.64 (d, J=8.5 Hz, 1H), 7.59 (s, 1H), 7.51 (s, 1H), 7.40 (d, J=7.5 Hz, 1H), 7.32 (d, J=5.6 Hz, 1H), 6.74 (s, 1H), 6.43 (s, 1H), 5.46 (d, J=6.8 Hz, 1H), 4.67 (d, J=3.0 Hz, 2H), 4.57-4.52 (m, 1H), 4.46 (d, J=5.5 Hz, 2H), 2.69-2.61 (m, 8H), 2.05-1.93 (m, 1H), 1.56 (d, J=6.7 Hz, 3H). LC/MS (ESI) m/z: 577 (M+H)⁺. RT (Method A): 1.05 min.

Compound 444 was prepared based on Scheme 118:

| # | Reactant A | Reactant B | Reactant C | Characterization Data |
|---|---|---|---|---|
| 444ª | 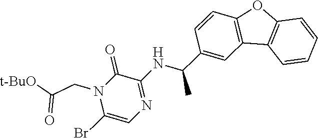 | 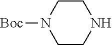 [SMILES: CC(C)(C)OC(=O)N1CCNCC1] | 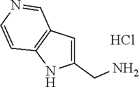 [SMILES: NCc1cc2cnccc2[nH]1 ; Cl] | ¹H NMR (400 MHz, DMSO-d₆) δ 11.39 (s, 1H), 8.79 (t, J = 5.3 Hz, 1H), 8.74 (s, 1H), 8.22 (s, 2H), 8.11 (d, J = 6.1 Hz, 2H), 7.68 (d, J = 8.2 Hz, 1H), 7.62 (d, J = 8.4 Hz, 1H), 7.60-7.57 (m, 1H), 7.53-7.48 (m, 1H), 7.39 (t, J = 7.5 Hz, 1H), 7.33 (d, J = 7.0 Hz, 2H), 6.61 (s, 1H), 6.43 (s, 1H), 5.26-5.20 (m, 1H), 4.69 (d, J = 3.3 Hz, 2H), 4.45 (d, J = 5.4 Hz, 2H), 2.89-2.79 (m, 4H), 2.69- 2.59 (m, 4H), 1.58 (d, J = 7.0 Hz, 3H). LC/MS (ESI) m/z: 577 (M + H)⁺. RT (Method A): 1.07 min. |

^{a}Step 1 was performed in the presence of CsF in DMSO.

### Scheme 119. Synthesis of (N-((1H-pyrrolo[3,2-c]pyridine-2-yl)methyl)-2-(5-(((R)-1-(dibenzo[b,d]furan-2-yl)ethyl)amino)-2-(4-(((3R,3aR,6R,6aR)-6-hydroxyhexahydrofuro[3,2-b]furan-3-yl)oxy)phenyl)-6-oxopyrimidin-1(6H)-yl) acetamide (Compound 377)

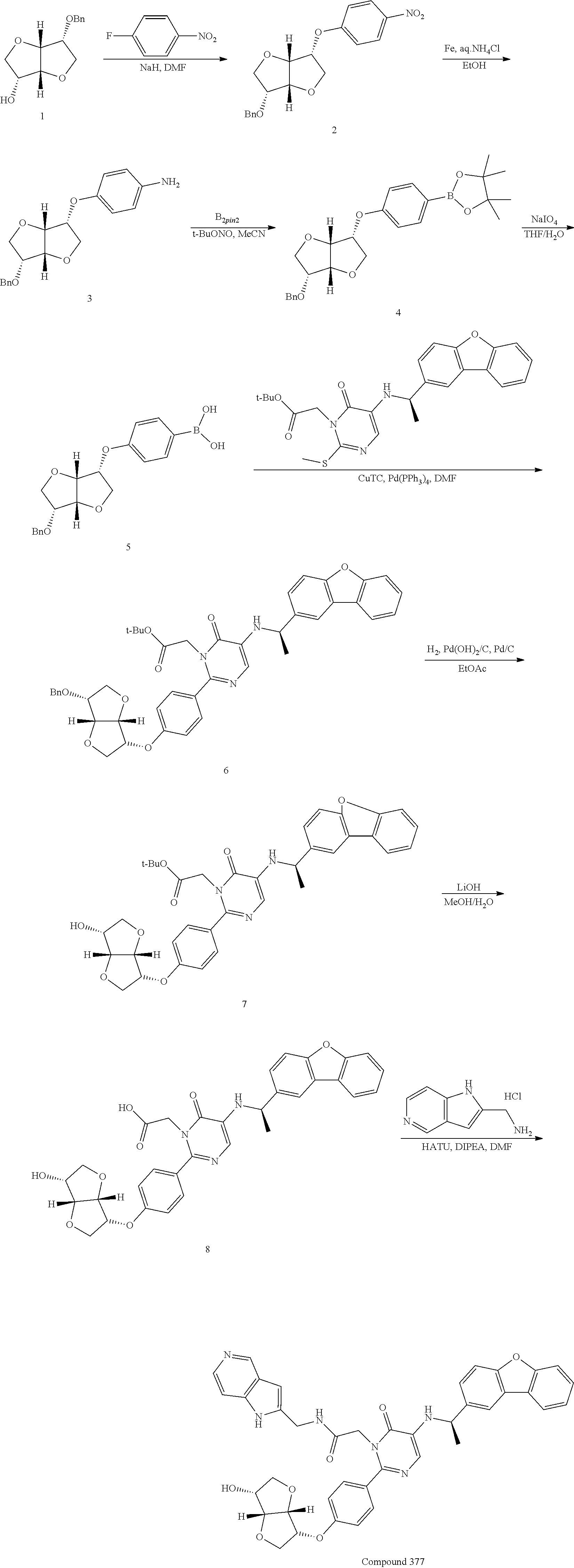

### Step 1: (3R,3aR,6R,6aR)-3-(benzyloxy)-6-(4-nitrophenoxy)hexahydrofuro[3,2-b]furan (2)

To a solution of (3R,3aR,6R,6aR)-6-(benzyloxy)hexahydrofuro[3,2-b]furan-3-ol (2.0 g, 8.47 mmol) in dry DMF (20 mL) was added NaH (245 mg, 10.21 mmol, 60% dispersion in mineral oil) in portions at 0° C. and the mixture was stirred at 0° C. for 0.5 hour. And then, to the reaction mixture was added 1-fluoro-4-nitrobenzene (1.55 g, 10.99 mmol) dropwise at 0° C. and the reaction mixture was stirred at room temperature for 3 hours. The mixture was quenched with saturated aq. NH₄Cl solution and extracted with EtOAc twice. The combined organic layers were washed with brine, dried over anhydrous Na₂SO₄, filtered, and concentrated to dryness. The residue was purified by flash chromatography (silica gel, 0-40% EtOAc in PE) to give the title compound (2.5 g, yield 82.6%) as a yellow oil. LC/MS (ESI) m/z: 358 (M+H)⁺.

### Step 2: 4-(((3R,3aR,6R,6aR)-6-(Benzyloxy)hexahydrofuro[3,2-b]furan-3-yl)oxy) aniline (3)

To a solution of (3R,3aR,6R,6aR)-3-(benzyloxy)-6-(4-nitrophenoxy)hexahydrofuro[3,2-b]furan (2.5 g, 7.00 mmol) in EtOH (25 mL) and saturated aq. NH₄Cl solution (5 mL) was added Fe (785 mg, 14.02 mmol) under N₂ atmosphere, and the reaction mixture was stirred at 80° C. overnight. The mixture was diluted with EtOAc and filtered. The filtrate was washed with water and brine, dried over anhydrous Na₂SO₄, filtered, and concentrated under reduced pressure to dryness. The residue was purified by flash chromatography (silica gel, 0-100% EtOAc in PE) to give the title compound (1.9 g, yield 82.9%) as a yellow oil. LC/MS (ESI) m/z: 328 (M+H)⁺.

### Step 3: 2-(4-(((3R,3aR,6R,6aR)-6-(Benzyloxy)hexahydrofuro[3,2-b]furan-3-yl)oxy) phenyl)-4,4,5,5-tetramethyl-1,3,2-dioxaborolane (4)

To a solution of 4-(((3R,3aR,6R,6aR)-6-(benzyloxy)hexahydrofuro[3,2-b]furan-3-yl)oxy)aniline (1.9 g, 5.8 mmol) in MeCN (20 mL) was added t-BuONO (2.99 g, 29.0 mmol) under N₂ atmosphere and the reaction mixture was stirred at 0° C. for 10 minutes. Then 4,4,4′,4′,5,5,5′,5′-octamethyl-2,2′-bi(1,3,2-dioxaborolane) (4.42 g, 17.41 mmol) was added into the above mixture and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give the title compound (2.35 g, yield 92.3%) as a yellow oil. LC/MS (ESI) m/z: 439 (M+H)⁺.

### Step 4: (4-(((3R,3aR,6R,6aR)-6-(Benzyloxy)hexahydrofuro[3,2-b]furan-3-yl)oxy) phenyl)boronic acid (5)

To a solution of 2-(4-(((3R,3aR,6R,6aR)-6-(benzyloxy)hexahydrofuro[3,2-b]furan-3-yl)oxy)phenyl)-4,4,5,5-tetramethyl-1,3,2-dioxaborolane (2.3 g, 5.25 mol) in THE/water (25 mL, v/v=4/1) was added NaIO₄ (5.61 g, 26.2 mmol) at 0° C. and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) the title compound (1.16 g, yield 62.0%) as a yellow solid. LC/MS (ESI) m/z: 357 (M+H)⁺.

### Step 5: Tert-butyl 2-(2-(4-(((3R,3aR,6R,6aR)-6-(benzyloxy)hexahydrofuro[3,2-b]furan-3-yl)oxy)phenyl)-5-(((R)-1-(dibenzo[b,d]furan-2-yl)ethyl)amino)-6-oxopyrimidin-1(6H)-yl)acetate (6)

To a mixture of tert-butyl (R)-2-(5-((1-(dibenzo[b,d]furan-2-yl)ethyl)amino)-2-(methylthio)-6-oxopyrimidin-1(6H)-yl)acetate (300 mg, 0.64 mmol) and (4-(((3R,3aR,6R,6aR)-6-(benzyloxy)hexahydrofuro[3,2-b]furan-3-yl)oxy)phenyl)boronic acid (350 mg, 0.98 mmol) in DMF (5 mL) was added CuTC (271 mg, 1.41 mmol) and Pd(PPh₃)₄ (150 mg, 0.13 mmol) at room temperature under N₂ atmosphere, the reaction mixture was degassed under N₂ atmosphere for three times and stirred at 80° C. for 16 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (310 mg, yield 65.9%) as a colorless oil. LC/MS (ESI) m/z: 730 (M+H)⁺.

### Step 6: Tert-butyl 2-(5-(((R)-1-(dibenzo[b,d]furan-2-yl)ethyl)amino)-2-(4-(((3R,3aR,6R,6aR)-6-hydroxyhexahydrofuro[3,2-b]furan-3-yl)oxy)phenyl)-6-oxopyrimidin-1(6H)-yl)acetate (7)

To a solution of tert-butyl 2-(2-(4-(((3R,3aR,6R,6aR)-6-(benzyloxy)hexahydrofuro[3,2-b]furan-3-yl)oxy)phenyl)-5-(((R)-1-(dibenzo[b,d]furan-2-yl)ethyl)amino)-6-oxopyrimidin-1(6H)-yl)acetate (310 mg, 0.42 mmol) in EtOAc (5 mL) was added Pd/C (20 mg, 10% wt.) and Pd(OH)₂ (20 mg, 10% wt.), the reaction mixture was degassed under N₂ atmosphere for three times and stirred under a H₂ balloon at 50° C. for 3 days. The mixture was filtered, and the filtrate was concentrated to dryness. The residue was purified by prep-HPLC to give the title compound (55 mg, yield 20.2%) as a white solid. LC/MS (ESI) m/z: 640 (M+H)⁺.

### Step 7: 2-(5-(((R)-1-(dibenzo[b,d]furan-2-yl)ethyl)amino)-2-(4-(((3R,3aR,6R,6aR)-6-hydroxyhexahydrofuro[3,2-b]furan-3-yl)oxy)phenyl)-6-oxopyrimidin-1(6H)-yl)acetic acid (8)

To a solution of tert-butyl 2-(5-(((R)-1-(dibenzo[b,d]furan-2-yl)ethyl)amino)-2-(4-(((3R,3aR,6R,6aR)-6-hydroxyhexahydrofuro[3,2-b]furan-3-yl)oxy)phenyl)-6-oxopyrimidin-1(6H)-yl)acetate (55 mg, 0.086 mmol) in MeOH/water (2.0 mL, v/v=4/1) was added LiOH·H₂O (7 mg, 0.17 mmol) and the reaction mixture was stirred at 60° C. overnight. The mixture was acidified with 1N aq. HCl to pH˜3 and extracted with EtOAc twice. The combined organic layers were dried over anhydrous Na₂SO₄, filtered, and concentrated under reduced pressure to give the title compound (50 mg, yield 99.6%) as a white solid, which was used directly in the next step without further purification. LC/MS (ESI) m/z: 584 (M+H)⁺.

### Step 8: N-((1H-pyrrolo[3,2-c]pyridine-2-yl)methyl)-2-(5-(((R)-1-(dibenzo[b,d]furan-2-yl)ethyl)amino)-2-(4-(((3R,3aR,6R,6aR)-6-hydroxyhexahydrofuro[3,2-b]furan-3-yl)oxy)phenyl)-6-oxopyrimidin-1(6H)-yl)acetamide (Compound 377)

To a mixture of 2-(5-(((R)-1-(dibenzo[b,d]furan-2-yl)ethyl)amino)-2-(4-(((3R,3aR,6R,6aR)-6-hydroxyhexahydrofuro[3,2-b]furan-3-yl)oxy)phenyl)-6-oxopyrimidin-1(6H)-yl)acetic acid (50 mg, 0.085 mmol) and (1H-pyrrolo[3,2-c]pyridine-2-yl)methanamine hydrochloride (20 mg, 0.10 mmol) in DMF (2 mL) was added DIPEA (55 mg, 0.42 mmol) and HATU (39 mg, 0.10 mmol) and the reaction mixture was stirred at room temperature for 1 hour. The mixture was concentrated under reduced pressure to dryness. The residue was purified by prep-HPLC to give Compound 377 (7.2 mg, yield 11.8%) as a white solid. ¹H NMR (400 MHz, DMSO-d₆) δ 11.32 (s, 1H), 8.75 (s, 1H), 8.70 (t, J=5.5 Hz, 1H), 8.22 (s, 1H), 8.11 (t, J=7.2 Hz, 2H), 7.70-7.65 (m, 2H), 7.62 (dd, J=8.6, 1.6 Hz, 1H), 7.54-7.49 (m, 1H), 7.40 (t, J=7.5 Hz, 1H), 7.34 (d, J=5.7 Hz, 1H), 7.29 (d, J=8.8 Hz, 2H), 6.97 (s, 1H), 6.89 (d, J=8.8 Hz, 2H), 6.36 (s, 1H), 5.86 (d, J=7.1 Hz, 1H), 4.90 (d, J=6.8 Hz, 1H), 4.77 (d, J=5.2 Hz, 1H), 4.66 (m, 2H), 4.55-4.42 (m, 4H), 4.33 (t, J=4.9 Hz, 1H), 4.12-4.06 (m, 1H), 4.01 (m, 1H), 3.76-3.69 (m, 2H), 3.36 (d, J=8.5 Hz, 1H), 1.60 (d, J=6.7 Hz, 3H). LC/MS (ESI) m/z: 713 (M+H)⁺. RT (Method A): 1.51 min.

### Scheme 120. Synthesis of N-((1H-pyrrolo[3,2-c]pyridine-2-yl)methyl)-2-(2-(2-azabicyclo[2.2.1]heptan-2-yl)-5-(((R)-1-(dibenzo[b,d]furan-2-yl)ethyl)amino)-6-oxopyrimidin-1(6H)-yl)acetamide (Compound 383)

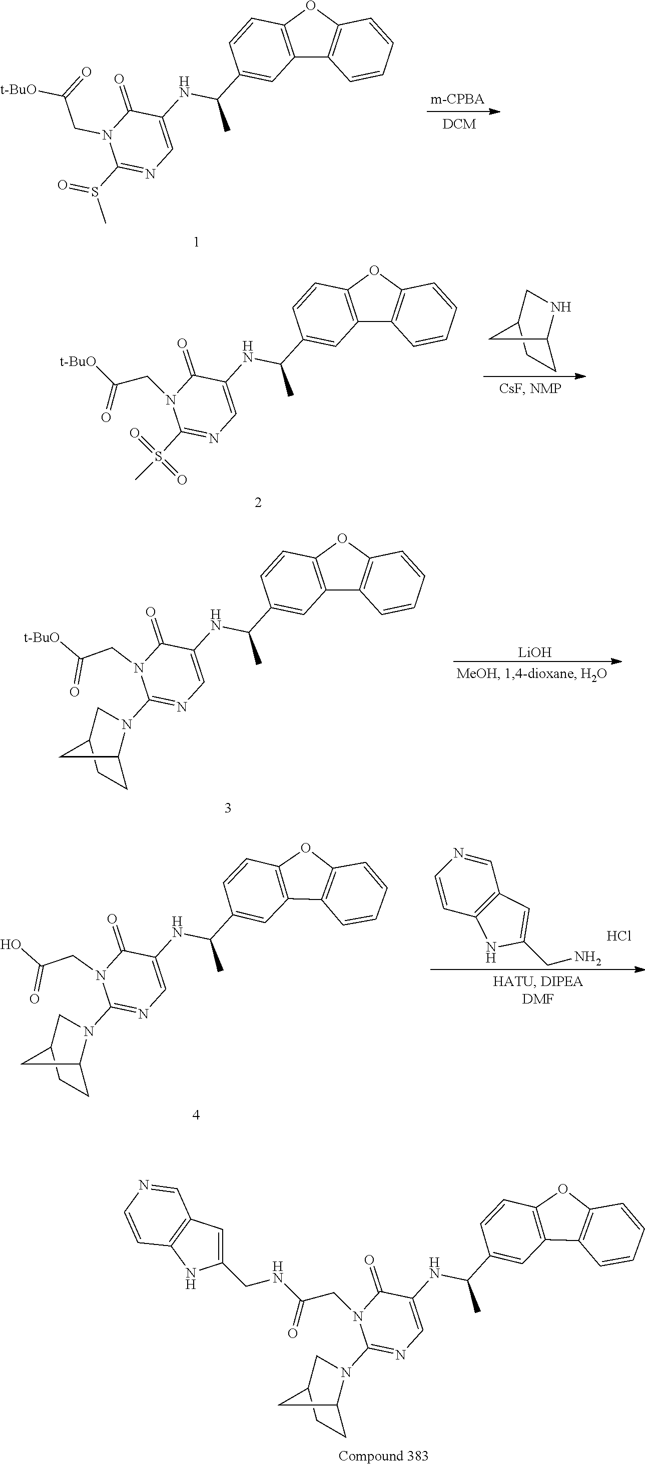

### Step 1: Tert-butyl I-2-(5-(((R)-(dibenzo[b,d]furan-2-yl)ethyl)amino)-2-(methylsulfonyl)-6-oxopyrimidin-1(6H)-yl)acetate (2)

To a solution of tert-butyl 2-(5-(((R)-1-(dibenzo[b,d]furan-2-yl)ethyl)amino)-2-(methylsulfinyl)-6-oxopyrimidin-1(6H)-yl)acetate (910 mg, 1.89 mmol) in DCM (10 mL) was added m-CPBA (980 mg, 5.68 mmol) and the reaction mixture was stirred at room temperature for 2 hours. The mixture was diluted with EtOAc, washed with saturated aq. Na₂S₂O₃ solution and brine, dried over anhydrous Na₂SO₄, filtered, and concentrated under reduced pressure to dryness. The residue was purified by flash chromatography (silica gel, 0-18% EtOAc in PE) to give the title compound (296 mg, yield 31.5%) as red solid. LC/MS (ESI) m/z: 498 (M+H)⁺.

### Step 2: Tert-butyl 2-(2-(2-azabicyclo[2.2.1]heptan-2-yl)-5-(((R)-1-(dibenzo[b,d]furan-2-yl)ethyl)amino)-6-oxopyrimidin-1(6H)-yl)acetate (3)

To a mixture of tert-butyl (R)-2-(5-((1-(dibenzo[b,d]furan-2-yl)ethyl)amino)-2-(methylsulfonyl)-6-oxopyrimidin-1(6H)-yl)acetate (60 mg, 0.12 mmol) and 2-azabicyclo[2.2.1]heptane (140 mg, 1.44 mmol) in NMP (2 mL) was added CsF (145 mg, 0.95 mmol) under N₂ atmosphere. The mixture was degassed under N₂ atmosphere for three times and stirred in a sealed tube at 130° C. for 3 hours. The reaction mixture was diluted with EtOAc and filtered. The filtrate was washed with water and brine, dried over anhydrous Na₂SO₄, filtered, and concentrated under reduced pressure to dryness. The residue was purified by flash chromatography (silica gel, 0-20% EtOAc in PE) to give the title compound (55 mg, yield 88.6%) as a white solid. ¹H NMR (400 MHz, CD₃OD) δ 8.05-8.00 (m, 2H), 7.57-7.53 (m, 2H), 7.52-7.46 (m, 2H), 7.35 (t, J=7.4 Hz, 1H), 6.72 (s, 1H), 4.78-4.72 (m, 1H), 4.65 (d, J=17.3 Hz, 1H), 4.59-4.52 (m, 1H), 3.89 (d, J=11.3 Hz, 1H), 3.40-3.36 (m, 1H), 2.85 (t, J=8.0 Hz, 1H), 2.51-2.47 (m, 1H), 1.80-1.73 (m, 1H), 1.64-1.60 (m, 5H), 1.47 (d, J=7.1 Hz, 9H), 1.40-1.33 (m, 2H), 1.25 (t, J=7.1 Hz, 1H). LC/MS (ESI) m/z: 515 (M+H)⁺.

### Step 3: 2-(2-(2-azabicyclo[2.2.1]heptan-2-yl)-5-(((R)-1-(dibenzo[b,d]furan-2-yl)ethyl) amino)-6-oxopyrimidin-1(6H)-yl)acetic acid (4)

To a solution of tert-butyl 2-(2-(2-azabicyclo[2.2.1]heptan-2-yl)-5-((I-1-(dibenzo[b,d]furan-2-yl)ethyl)amino)-6-oxopyrimidin-1(6H)-yl)acetate (55 mg, 0.11 mmol) in MeOH/1,4-dioxane/water (5 mL, v/v/v=2/2/1) was added LiOH (10 mg, 0.42 mmol) and the mixture was stirred at 65° C. overnight. The mixture was acidified with 1N aq. HCl to pH˜5 and extracted with EtOAc twice. The combined organic layers were washed with brine, dried over anhydrous Na₂SO₄, filtered, and concentrated under reduced pressure to dryness to give the title compound (35 mg, yield 71.4%) as a white solid, which was used directly in the next step without further purification. LC/MS (ESI) m/z: 459 (M+H)⁺.

### Step 4: N-((1H-pyrrolo[3,2-c]pyridine-2-yl)methyl)-2-(2-(2-azabicyclo[2.2.1]heptan-2-yl)-5-((I-1-(dibenzo[b,d]furan-2-yl)ethyl)amino)-6-oxopyrimidin-1(6H)-yl)acetamide (Compound 383)

To a mixture of 2-(2-(2-azabicyclo[2.2.1]heptan-2-yl)-5-((I-1-(dibenzo[b,d]furan-2-yl)ethyl)amino)-6-oxopyrimidin-1(6H)-yl)acetic acid (35 mg, 0.076 mmol) and (1H-pyrrolo[3,2-c]pyridine-2-yl)methanamine hydrochloride (20 mg, 0.11 mmol) in DMF (2 mL) was added DIPEA (39 mg, 0.30 mmol) and HATU (35 mg, 0.092 mmol) and the reaction mixture was stirred at room temperature for 0.5 hour. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=9:1) and further purified by prep-HPLC to give Compound 383 (7.1 mg, yield 16.0%) as a white solid. ¹H NMR (400 MHz, DMSO-d₆) δ 11.31 (s, 1H), 8.78-8.70 (m, 2H), 8.16 (d, J=3.8 Hz, 1H), 8.10 (t, J=6.0 Hz, 2H), 7.70-7.61 (m, 2H), 7.60-7.55 (m, 1H), 7.50 (t, J=7.7 Hz, 1H), 7.38 (t, J=7.5 Hz, 1H), 7.32 (d, J=5.6 Hz, 1H), 6.69 (d, J=4.1 Hz, 1H), 6.43 (s, 1H), 5.15 (d, J=6.6 Hz, 1H), 4.69-4.53 (m, 2H), 4.52-4.45 (m, 3H), 3.88 (d, J=8.8 Hz, 1H), 3.31-3.27 (m, 1H), 2.79-2.71 (m, 1H), 2.36-2.30 (m, 1H), 1.72-1.59 (m, 1H), 1.54 (d, J=6.6 Hz, 3H), 1.47-1.36 (m, 3H), 1.25-1.15 (m, 2H). LC/MS (ESI) m/z: 588 (M+H)⁺. RT (Method A): 1.80 min.

Compound 491 was prepared based on Scheme 120:

| # | Reactant A | Reactant B | Reactant C | Characterization Data |
|---|---|---|---|---|
| 491^{a} | 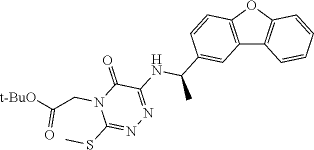 | 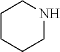 | 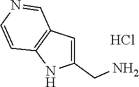 [SMILES: NCc1cc2cnccc2[nH]1 ; Cl] | ¹H NMR (400 MHz, CD₃OD) δ 8.69 (s, 1H), 8.11-8.07 (m, 2H), 8.00 (d, J = 7.7 Hz, 1H), 7.54 (t, J = 5.2 Hz, 2H), 7.50 (d, J = 8.5 Hz, 1H), 7.48-7.43 (m, 1H), 7.37- 7.32 (m, 2H), 6.56 (s, 1H), 5.24 (q, J = 7.0 Hz, 1H), 4.73 (s, 2H), 4.58 (s, 2H), 2.92- 2.85 (m, 4H), 1.67 (d, J = 6.9 Hz, 3H), 1.51- 1.48 (m, 4H), 1.22-1.18 (m, 2H). LC/MS (ESI) m/z: 577 (M + H)⁺. RT (Method A): 1.80 min. |

^{a}Step 2 was performed in DMSO.

### Scheme 121. Synthesis of (R)—N-((1H-pyrrolo[3,2-c]pyridine-2-yl)methyl)-2-(6-((1-(dibenzo[b,d]furan-2-yl)ethyl)amino)-3-(2-fluorophenyl)-5-oxo-1,2,4-triazin-4(5H)-yl)acetamide

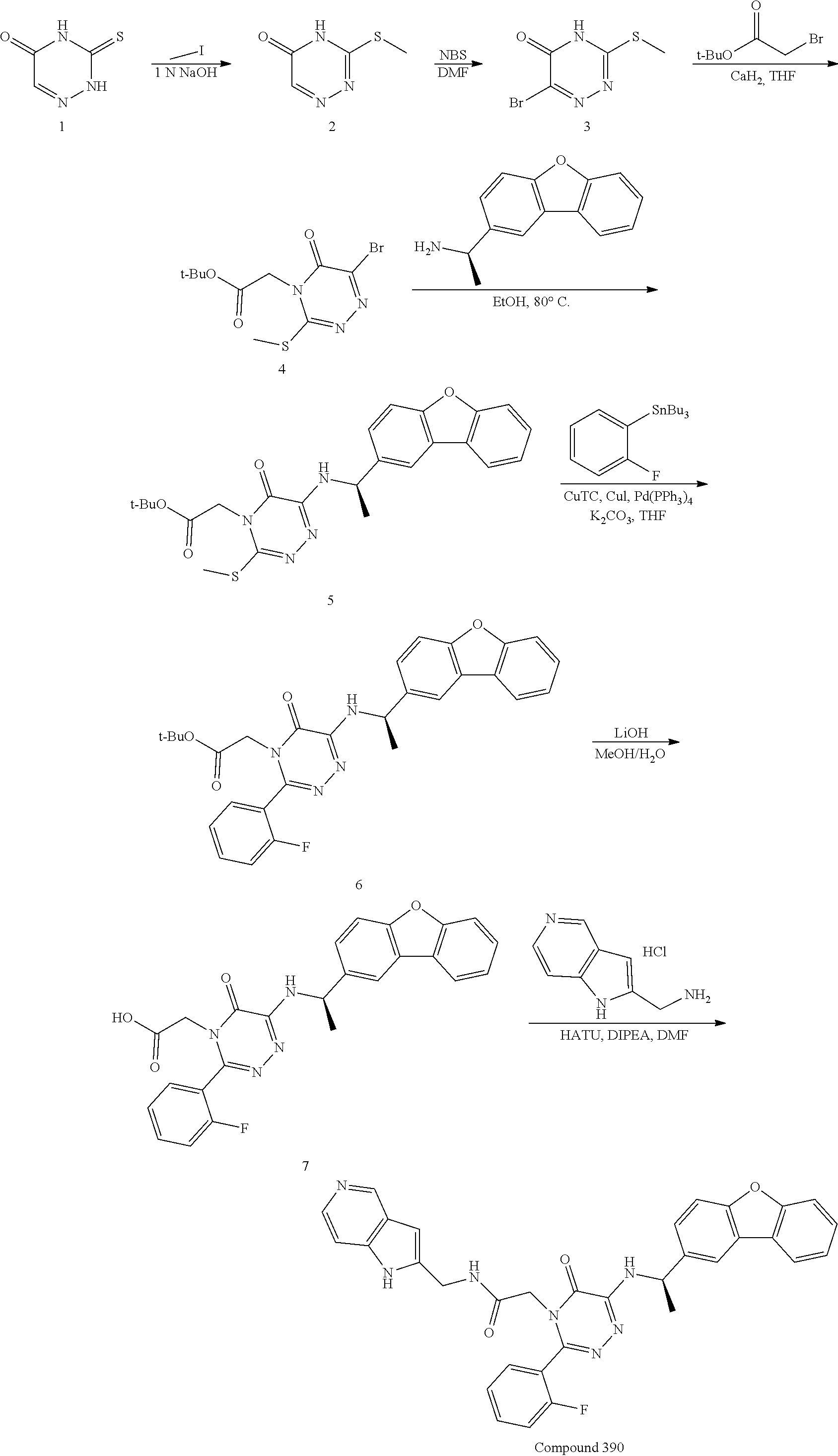

### Step 1: 3-(Methylthio)-1,2,4-triazin-5(4H)-one (2)

To a solution of 3-thioxo-3,4-dihydro-1,2,4-triazin-5(2H)-one (3 g, 23.3 mmol) in 1 N aq. NaOH (26 mL) was added iodomethane (2.2 mL, 34.8 mmol) dropwise at 0° C. under N₂ atmosphere and the mixture was stirred at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (1.9 g, yield 56.5%) as a white solid. LC/MS (ESI) m/z: 144 (M+H)⁺.

### Step 2: 6-Bromo-3-(methylthio)-1,2,4-triazin-5(4H)-one (3)

To a solution of 3-(methylthio)-1,2,4-triazin-5(4H)-one (1.9 g, 13.3 mmol) in DMF (20 mL) was added NBS (2.6 g, 14.6 mmol) in portions and the mixture was stirred under N₂ atmosphere at room temperature overnight. The mixture was quenched with saturated aq. Na₂S₂O₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (2.0 g, yield 67.8%) as a yellow solid. LC/MS (ESI) m/z: 222 (M+H)⁺.

### Step 3: Tert-butyl 2-(6-bromo-3-(methylthio)-5-oxo-1,2,4-triazin-4(5H)-yl)acetate (4)

To a mixture of 6-bromo-3-(methylthio)-1,2,4-triazin-5(4H)-one (1.0 g, 4.53 mmol) and tert-butyl 2-bromoacetate (1.32 g, 6.79 mmol) in THF (10 mL) was added CaH₂ (1.14 g, 27.21 mmol) in portions at 0° C. and the mixture was stirred under N₂ atmosphere at 70° C. overnight. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-15% EtOAc in PE) to give the title compound (330 mg, yield 21.7%) as a white solid. LC/MS (ESI) m/z: 336 (M+H)⁺. ¹H NMR (400 MHz, CDCl₃) δ 4.66 (s, 2H), 2.70 (s, 3H), 1.48 (s, 9H).

### Step 4: Tert-butyl (R)-2-(6-((1-(dibenzo[b,d]furan-2-yl)ethyl)amino)-3-(methylthio)-5-oxo-1,2,4-triazin-4(5H)-yl)acetate (5)

To a solution of tert-butyl 2-(6-bromo-3-(methylthio)-5-oxo-1,2,4-triazin-4(5H)-yl) acetate (200 mg, 0.60 mmol) in EtOH (2 mL) was added I-1-(dibenzo[b,d]furan-2-yl)ethan-1-amine (164 mg, 0.78 mmol) and the mixture was stirred at 80° C. overnight. The mixture was concentrated under reduced pressure to dryness. The residue was purified by flash chromatography (silica gel, 0-20% EtOAc in PE) to give the title compound (105 mg, yield 37.5%) as a white solid. LC/MS (ESI) m/z: 467 (M+H)⁺.

### Step 5: Tert-butyl (R)-2-(6-((1-(dibenzo[b,d]furan-2-yl)ethyl)amino)-3-(2-fluorophenyl)-5-oxo-1,2,4-triazin-4(5H)-yl)acetate (6)

To a mixture of tert-butyl (R)-2-(6-((1-(dibenzo[b,d]furan-2-yl)ethyl)amino)-3-(methylthio)-5-oxo-1,2,4-triazin-4(5H)-yl)acetate (100 mg, 0.21 mmol) and tributyl(2-fluorophenyl)stannane (402 mg, 1.05 mmol) in THF (3 mL) was added K₂CO₃ (87 mg, 0.63 mmol), CuI (21 mg, 0.11 mmol), CuTC (120 mg, 0.63 mmol) and Pd(PPh₃)₄ (127 mg, 0.11 mmol) under N₂ atmosphere, the reaction mixture was degassed under N₂ atmosphere for three times and stirred at 70° C. overnight. The mixture was diluted with water and extracted with EtOAc twice. The combined organic layers were washed with saturated aq. NaHCO₃ solution and brine, dried over anhydrous Na₂SO₄, filtered, and concentrated under reduced pressure to dryness. The residue was purified by flash chromatography (silica gel, 0-30% EtOAc in PE) to give the title compound (26 mg, yield 24.1%) as a yellow oil. LC/MS (ESI) m/z: 515 (M+H)⁺.

### Step 6: (R)-2-(6-((1-(dibenzo[b,d]furan-2-yl)ethyl)amino)-3-(2-fluorophenyl)-5-oxo-1,2,4-triazin-4(5H)-yl)acetic acid (7)

To a solution of tert-butyl (R)-2-(6-((1-(dibenzo[b,d]furan-2-yl)ethyl)amino)-3-(2-fluorophenyl)-5-oxo-1,2,4-triazin-4(5H)-yl)acetate (26 mg, 0.05 mmol) in MeOH/water (1 mL, v/v=4/1) was added LiOH (4 mg, 0.15 mmol) and the mixture was stirred at 60° C. for 5 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (20 mg, yield 87.0%) as a yellow solid, which was used directly in the next step without further purification. LC/MS (ESI) m/z: 459 (M+H)⁺.

### Step 7: (R)—N-((1H-pyrrolo[3,2-c]pyridine-2-yl)methyl)-2-(6-((1-(dibenzo[b,d]furan-2-yl)ethyl)amino)-3-(2-fluorophenyl)-5-oxo-1,2,4-triazin-4(5H)-yl)acetamide (Compound 390)

To a mixture of (R)-2-(6-((1-(dibenzo[b,d]furan-2-yl)ethyl)amino)-3-(2-fluorophenyl)-5-oxo-1,2,4-triazin-4(5H)-yl)acetic acid (20 mg, 0.044 mmol) and (1H-pyrrolo[3,2-c]pyridine-2-yl)methanamine hydrochloride (10 mg, 0.057 mmol) in DMF (1 mL) was added DIPEA (28 mg, 0.22 mmol) and HATU (22 mg, 0.057 mmol) and the reaction mixture was stirred at room temperature for 1 hour. The mixture was concentrated under reduced pressure to dryness. The residue was purified by prep-HPLC to give Compound 390 (3.0 mg, yield 11.6%) as a white solid. ¹H NMR (400 MHz, CD₃OD) δ 8.74 (s, 1H), 8.22-8.05 (m, 2H), 8.02 (d, J=7.7 Hz, 1H), 7.58 (d, J=5.4 Hz, 1H), 7.53 (d, J=9.0 Hz, 2H), 7.50-7.37 (m, 4H), 7.33 (d, J=7.4 Hz, 1H), 7.19 (t, J=8.9 Hz, 1H), 7.10 (s, 1H), 6.43 (s, 1H), 5.43-5.36 (m, 1H), 4.69-4.55 (m, 1H), 4.43 (s, 2H), 4.32-4.18 (m, 1H), 1.72 (d, J=6.9 Hz, 3H). LC/MS (ESI) m/z: 588 (M+H)⁺.

RT (Method A): 1.54 min.

### Scheme 122. Synthesis of N-((1H-pyrrolo[3,2-c]pyridine-2-yl)methyl)-2-(5-((1-(dibenzo[b,d]furan-2-yl)cyclopropyl)amino)-2-(2-fluorophenyl)-6-oxopyrimidin-1(6H)-yl)acetamide (Compound 403)

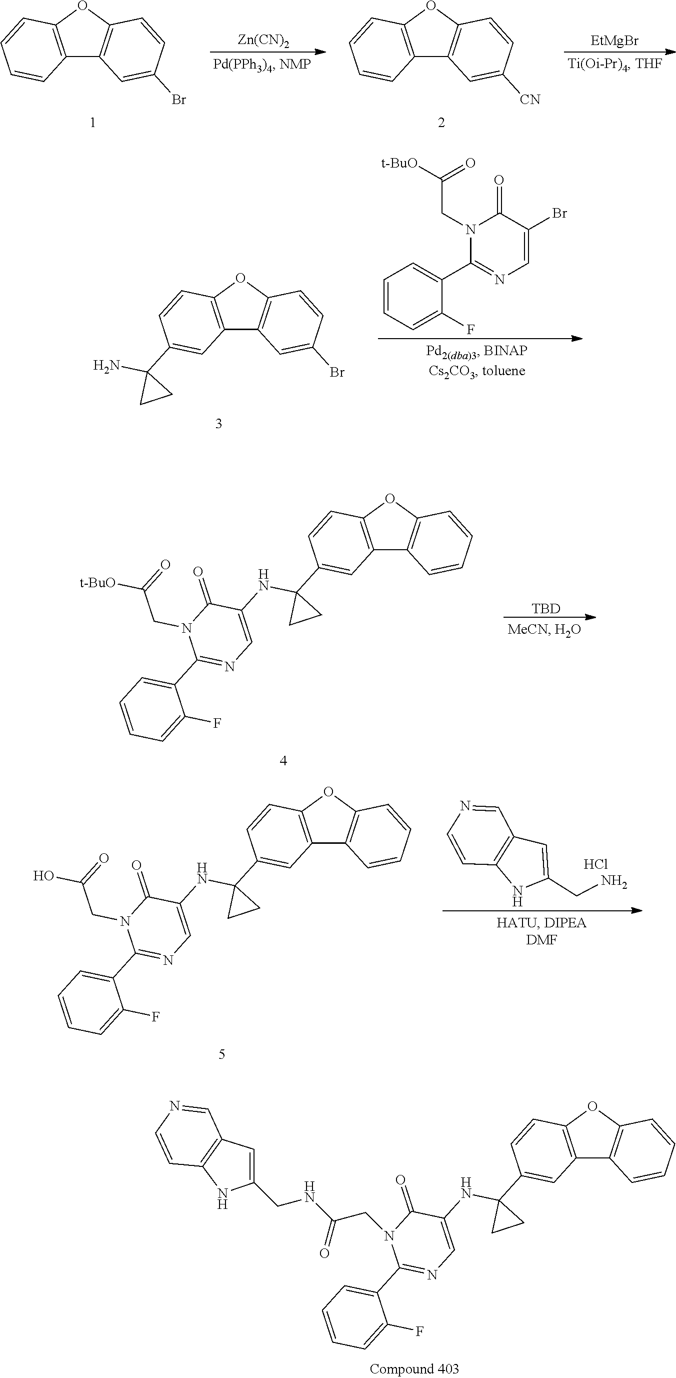

### Step 1: Dibenzo[b,d]furan-2-carbonitrile (2)

To a solution of 2-bromodibenzo[b,d]furan (5.0 g, 20.24 mmol) in NMP (50 mL) was added Zn(CN)₂ (4.75 g, 40.49 mmol) and Pd(PPh₃)₄ (2.34 g, 2.02 mmol) under N₂ atmosphere. The mixture was degassed under N₂ atmosphere for three times and stirred under N₂ atmosphere at 110° C. for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (2.4 g, yield 60.6%) as a white solid. LC/MS (ESI) m/z: 194 (M+H)⁺.

### Step 2: 1-(Dibenzo[b,d]furan-2-yl)cyclopropan-1-amine (3)

To a solution of dibenzo[b,d]furan-2-carbonitrile (2.0 g, 10.3 mmol) in THF (20 mL) were added Ti(Oi-Pr)₄ (3.23 g, 11.40 mmol) dropwise at −78° C. The mixture was warmed to −50° C. and stirred at −50° C. for 0.5 hours. EtMgBr (22.7 mL, 22.7 mmol, 1 M in THF) was added dropwise to the above mixture at −78° C. and the resulting mixture was stirred −78° C. to room temperature for 0.5 hours. The mixture was quenched with ice-water and extracted with EtOAc twice. The combined organic layers were washed with water and brine, dried over anhydrous Na₂SO₄, filtered, and concentrated under reduced pressure to dryness. The residue was purified by prep-HPLC to give the title compound (330 mg, yield 14.2%) as a white solid. LC/MS (ESI) (m/z): 224 (M+H)⁺.

### Step 3: Tert-butyl 2-(5-((1-(dibenzo[b,d]furan-2-yl)cyclopropyl)amino)-2-(2-fluorophenyl)-6-oxopyrimidin-1(6H)-yl)acetate (4)

To a mixture of 1-(dibenzo[b,d]furan-2-yl)cyclopropan-1-amine (80 mg, 0.35 mmol) and tert-butyl 2-(5-bromo-2-(2-fluorophenyl)-6-oxopyrimidin-1(6H)-yl)acetate (137 mg, 0.35 mmol) in toluene (3 mL) were added Pd₂(dba)₃ (66 mg, 0.07 mmol), BINAP (45 mg, 0.07 mmol) and Cs₂CO₃ (353 mg, 1.07 mmol) under N₂ atmosphere, the reaction mixture was degassed under N₂ atmosphere for three times and stirred at 120° C. overnight. The mixture was diluted with EtOAc, washed with water and brine, dried over anhydrous Na₂SO₄, filtered, and concentrated under reduced pressure to dryness. The residue was purified by prep-HPLC to give the title compound (32 mg, yield 17.1%) as a white solid. LC/MS (ESI) (m/z): 526 (M+H)⁺.

### Step 4: 2-(5-((1-(Dibenzo[b,d]furan-2-yl)cyclopropyl)amino)-2-(2-fluorophenyl)-6-oxopyrimidin-1(6H)-yl)acetic acid (5)

To a mixture of tert-butyl 2-(5-((1-(dibenzo[b,d]furan-2-yl)cyclopropyl)amino)-2-(2-fluorophenyl)-6-oxopyrimidin-1(6H)-yl)acetate (30 mg, 0.05 mmol) in MeCN (1 mL) and water (0.5 mL) was added TBD (15.8 mg, 0.11 mmol) and the mixture was stirred at room temperature overnight. The mixture was concentrated under reduced pressure to dryness to give the title compound (20 mg, yield 76.5%) as a white solid, which was directly used in the next reaction without purification. LC/MS (ESI) (m/z): 470 (M+H)⁺.

### Step 5: N-((1H-pyrrolo[3,2-c]pyridine-2-yl)methyl)-2-(5-((1-(dibenzo[b,d]furan-2-yl)cyclopropyl)amino)-2-(2-fluorophenyl)-6-oxopyrimidin-1(6H)-yl)acetamide (Compound 403)

To a mixture of 2-(5-((1-(dibenzo[b,d]furan-2-yl)cyclopropyl)amino)-2-(2-fluorophenyl)-6-oxopyrimidin-1(6H)-yl)acetic acid (20 mg, 0.04 mmol) and (1H-pyrrolo[3,2-c]pyridine-2-yl)methanamine hydrochloride (11 mg, 0.06 mmol) in DMF (2 mL) were added DIPEA (30.9 mg, 0.24 mmol) and HATU (22.8 mg, 0.06 mmol). The mixture was stirred at room temperature for 0.5 hour. The mixture was diluted with EtOAc, washed with water and brine, dried over anhydrous Na₂SO₄, filtered, and concentrated under reduced pressure to dryness. The residue was purified by prep-HPLC to give Compound 403 (2.0 mg, yield 8.0%) as a white solid. ¹H NMR (400 MHz, DMSO-d₆) δ 11.28 (s, 1H), 8.73 (s, 1H), 8.65-8.54 (m, 1H), 8.14-8.10 (m, 2H), 7.69-7.61 (m, 2H), 7.58-7.54 (m, 1H), 7.53-7.47 (m, 2H), 7.41-7.35 (m, 2H), 7.33-7.28 (m, 2H), 7.17 (s, 1H), 7.17-7.12 (m, 1H), 6.86 (s, 1H), 6.23 (s, 1H), 5.38-5.24 (m, 1H), 4.99-4.60 (m, 1H), 4.34 (d, J=5.6 Hz, 2H), 4.26-4.13 (m, 1H), 0.94-0.82 (m, 4H). LC/MS (ESI) (m/z): 599 (M+H)⁺. RT (Method A): 2.16 min.

### Scheme 123. Synthesis of N-(1-(2-(((1H-pyrrolo[3,2-c]pyridine-2-yl)methyl)amino)-2-oxoethyl)-2-methyl-6-oxo-1,6-dihydropyrimidin-5-yl)-2-phenylthiazole-4-carboxamide (Compound 406)

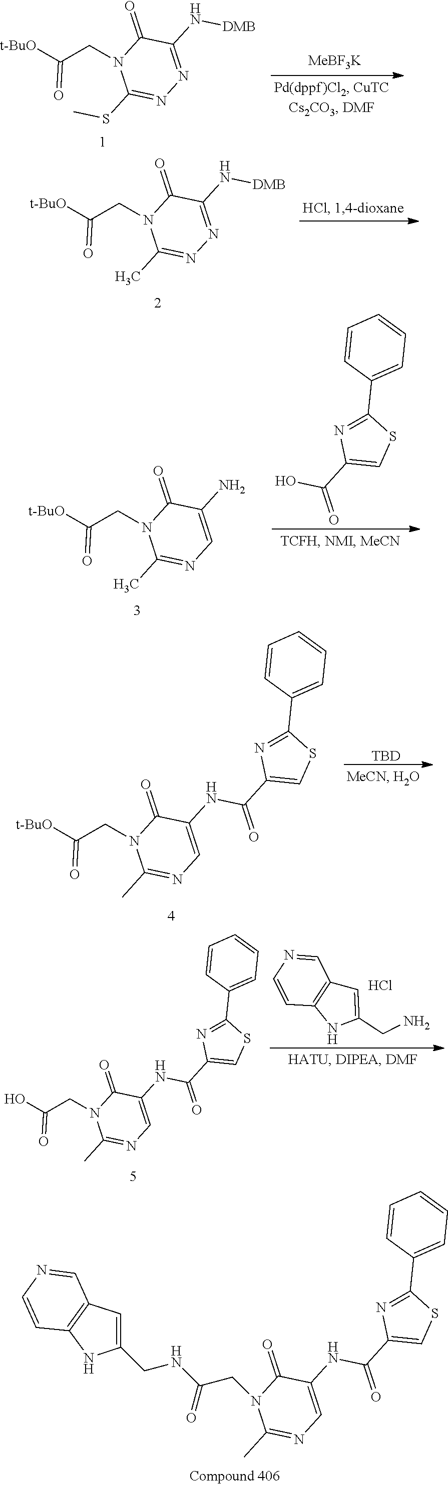

### Step 1: Tert-butyl 2-(5-((3,4-dimethylbenzyl)amino)-2-methyl-6-oxopyrimidin-1(6H)-yl)acetate (2)

To a mixture of tert-butyl 2-(5-((3,4-dimethylbenzyl)amino)-2-(methylthio)-6-oxopyrimidin-1(6H)-yl)acetate (1.0 g, 2.37 mmol) and MeBF₃K (2.89 g, 23.7 mmol) in DMF (10 mL) was added Cs₂CO₃ (1.93 g, 5.93 mmol), Pd(dppf)Cl₂ (350 mg, 0.47 mmol) and CuTC (990 mg, 5.21 mmol), the reaction mixture was degassed under N₂ atmosphere for three times and stirred at 100° C. overnight. The mixture was diluted with saturated aq. NaHCO₃ solution and extracted with EtOAc twice. The combined organic layers were washed with 1 N aq. HCl and brine, dried over anhydrous Na₂SO₄, filtered, and concentrated under reduced pressure to dryness. The residue was purified by flash chromatography (silica gel, 0-10% MeOH in DCM) to give the title compound (193 mg, yield 20.8%) as black oil. LC/MS (ESI) m/z: 390 (M+H)⁺.

### Step 2: Tert-butyl 2-(5-amino-2-methyl-6-oxopyrimidin-1(6H)-yl)acetate (3)

To a solution of tert-butyl 2-(5-((3,4-dimethylbenzyl)amino)-2-methyl-6-oxopyrimidin-1(6H)-yl)acetate (193 mg, 0.49 mmol) in HCl/1,4-dioxane (2 mL, 4M) and the mixture was stirred at room temperature for 5 hours. The mixture was diluted with EtOAc, washed with saturated aq. NaHCO₃ solution and brine, dried over anhydrous Na₂SO₄, filtered, and concentrated under reduced pressure to dryness to give the title compound (70 mg, yield 59.0%) as black oil, which was used directly in the next step without further purification. LC/MS (ESI) m/z: 240 (M+H)⁺.

### Step 3: Tert-butyl 2-(2-methyl-6-oxo-5-(2-phenylthiazole-4-carboxamido)pyrimidin-1(6H)-yl)acetate (4)

To a mixture of 2-phenylthiazole-4-carboxylic acid (70 mg, 0.292 mmol) and tert-butyl 2-(5-amino-2-methyl-6-oxopyrimidin-1(6H)-yl)acetate (60 mg, 0.292 mmol) in MeCN (2 mL) was added NMI (72 mg, 0.878 mmol) and TCFH (246 mg, 0.878 mmol), the mixture was stirred at room temperature for 4 hours. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-10% MeOH in DCM) to give the title compound (82 mg, yield 65.7%) as a brown oil. LC/MS (ESI) m/z: 427 (M+H)⁺.

### Step 4: 2-(2-methyl-6-oxo-5-(2-phenylthiazole-4-carboxamido)pyrimidin-1(6H)-yl) acetic acid (5)

To a solution of tert-butyl 2-(2-methyl-6-oxo-5-(2-phenylthiazole-4-carboxamido) pyrimidin-1(6H)-yl)acetate (82 mg, 0.192 mmol) in MeCN (1 mL) and water (1 mL) was added TBD (21 mg, 0.577 mmol) and the mixture was stirred at room temperature for 2 hours. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (48 mg, yield 67.3%) as a brown oil, which was used directly in the next step without further purification. LC/MS (ESI) m/z: 371 (M+H)⁺.

### Step 3: N-(1-(2-(((1H-pyrrolo[3,2-c]pyridine-2-yl)methyl)amino)-2-oxoethyl)-2-methyl-6-oxo-1,6-dihydropyrimidin-5-yl)-2-phenylthiazole-4-carboxamide (Compound 406)

To a mixture of 2-(2-methyl-6-oxo-5-(2-phenylthiazole-4-carboxamido)pyrimidin-1(6H)-yl)acetic acid (48 mg, 0.129 mmol) and (1H-pyrrolo[3,2-c]pyridine-2-yl) methanamine hydrochloride (20 mg, 0.129 mmol) in DMF (0.5 mL) was added DIPEA (83 mg, 0.641 mmol) and HATU (73 mg, 0.192 mmol), the mixture was stirred at room temperature for 1 hour. The mixture was concentrated under reduced pressure to dryness. The residue was purified by prep-HPLC to give Compound 406 (1.0 mg, yield 1.5%) as a white solid. ¹H NMR (400 MHz, CD₃OD) δ 8.96 (s, 1H), 8.70 (s, 1H), 8.37 (s, 1H), 8.09-8.06 (m, 2H), 8.03 (d, J=5.8 Hz, 1H), 7.54-7.52 (m, 3H), 7.37 (d, J=5.8 Hz, 1H), 6.59 (s, 1H), 4.96 (s, 2H), 4.65 (s, 2H), 2.57 (s, 3H). LC/MS (ESI) m/z: 500 (M+H)⁺. RT (Method A): 1.52 min.

### Scheme 124. Synthesis of (R)—N-((1H-pyrrolo[3,2-c]pyridine-2-yl)methyl)-2-(3-((1-(dibenzo[b,d]furan-2-yl)ethyl)amino)-6-morpholino-2-oxopyrazin-1(2H)-yl) acetamide (Compound 407)

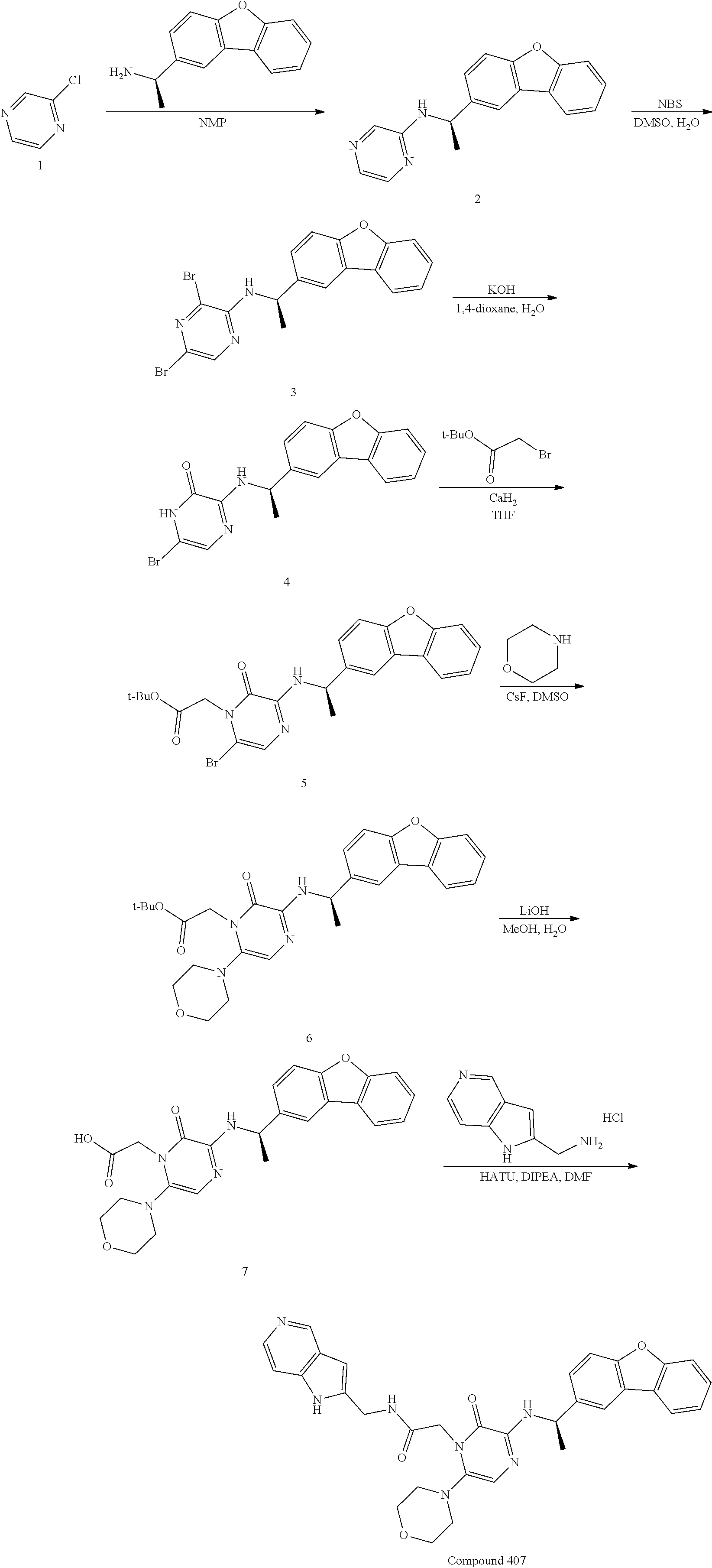

### Step 1: (R)—N-(1-(dibenzo[b,d]furan-2-yl)ethyl)pyrazin-2-amine (2)

To a solution of 2-chloropyrazine (2.84 g, 24.80 mmol) in NMP (20 mL) was added (R)-1-(dibenzo[b,d]furan-2-yl)ethan-1-amine (2.40 g, 11.36 mmol) at room temperature and the mixture was stirred in a sealed tube at 150° C. for 24 hours. The mixture was diluted with water and extracted with EtOAc twice. The combined organic layers were washed with water and brine, dried over anhydrous Na₂SO₄, filtered, and concentrated under reduced pressure to dryness. The residue was purified by flash chromatography (silica gel, 0-2% MeOH in DCM) to give the title compound (1.28 g, yield 39.0%) as a yellow oil. LC/MS (ESI) m/z: 290 (M+H)⁺.

### Step 2: (R)-3,5-dibromo-N-(1-(dibenzo[b,d]furan-2-yl)ethyl)pyrazin-2-amine (3)

To a solution of (R)—N-(1-(dibenzo[b,d]furan-2-yl)ethyl)pyrazin-2-amine (1.28 g, 4.42 mmol) in DMSO (20 mL) and water (0.8 mL) was added NBS (2.36 g, 13.26 mmol) at 50° C. under N₂ atmosphere and the reaction mixture was stirred at 50° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-16% EtOAc in PE) to give the title compound (600 mg, yield 30.4%) as a light-yellow oil. LC/MS (ESI) m/z: 448 (M+H)⁺.

### Step 3: (R)-6-bromo-3-((1-(dibenzo[b,d]furan-2-yl)ethyl)amino)pyrazin-2(1H)-one (4)

To a solution of (R)-3,5-dibromo-N-(1-(dibenzo[b,d]furan-2-yl)ethyl)pyrazin-2-amine (600 mg, 1.35 mmol) in 1,4-dioxane (8 mL) was added a solution of KOH (300 mg, 5.36 mmol) in water (6 mL) and the reaction mixture was stirred at 120° C. for 2 days. The mixture was diluted with water and extracted with DCM twice. The combined organic layers were washed with brine, dried over anhydrous Na₂SO₄, filtered, and concentrated under reduced pressure to dryness. The residue was purified by flash chromatography (silica gel, 0-26% EtOAc in PE) to give the title compound (270 mg, yield 52.2%) as a yellow solid. ¹H NMR (400 MHz, DMSO-d₆) δ 12.59 (s, 1H), 8.15-8.07 (m, 2H), 7.67 (d, J=8.2 Hz, 1H), 7.61 (d, J=8.5 Hz, 1H), 7.57-7.54 (m, 1H), 7.52-7.48 (m, 1H), 7.41-7.36 (m, 1H), 5.26-5.17 (m, 1H), 1.57 (d, J=7.0 Hz, 3H). LC/MS (ESI) m/z: 384/386 (M+H)⁺.

### Step 4: Tert-butyl (R)-2-(6-bromo-3-((1-(dibenzo[b,d]furan-2-yl)ethyl)amino)-2-oxopyrazin-1(2H)-yl)acetate (5)

To a mixture of (R)-6-bromo-3-((1-(dibenzo[b,d]furan-2-yl)ethyl)amino)pyrazin-2(1H)-one (210 mg, 0.55 mmol) and tert-butyl 2-bromoacetate (214 mg, 1.09 mmol) in THF (5 mL) was added CaH₂ (69 mg, 1.64 mmol) at 0° C. under N₂ atmosphere and the mixture was stirred at 60° C. overnight. The mixture was filtered, and the filtrate was concentrated to dryness. The residue was purified by flash chromatography (silica gel, 0-33% EtOAc in PE) to give the title compound (220 mg, yield 80.76%) as a yellow solid. LC/MS (ESI) m/z: 498 (M+H)⁺.

### Step 5: Tert-butyl (R)-2-(3-((1-(dibenzo[b,d]furan-2-yl)ethyl)amino)-6-morpholino-2-oxopyrazin-1(2H)-yl)acetate (6)

To a mixture of tert-butyl (R)-2-(6-bromo-3-((1-(dibenzo[b,d]furan-2-yl)ethyl)amino)-2-oxopyrazin-1(2H)-yl)acetate (210 mg, 0.42 mmol) and morpholine (498 mg, 5.72 mmol) in DMSO (5 mL) was added CsF (390 mg, 2.57 mmol) under N₂ atmosphere. The mixture was degassed under N₂ atmosphere for three times and stirred in a sealed tube at 120° C. for 3 hours. The reaction mixture was diluted with EtOAc and filtered. The filtrate was washed with saturated water and brine, dried over anhydrous Na₂SO₄, filtered, and concentrated under reduced pressure to dryness. The residue was purified by flash chromatography (silica gel, 0-40% EtOAc in PE) to give the title compound (106 mg, yield 49.9%) as a yellow oil. LC/MS (ESI) m/z: 505 (M+H)⁺.

### Step 6: (R)-2-(3-((1-(dibenzo[b,d]furan-2-yl)ethyl)amino)-6-morpholino-2-oxopyrazin-1(2H)-yl)acetic acid (7)

To a solution of tert-butyl (R)-2-(3-((1-(dibenzo[b,d]furan-2-yl)ethyl)amino)-6-morpholino-2-oxopyrazin-1(2H)-yl)acetate (106 mg, 0.21 mmol) in MeOH/water (5 mL, v/v=2/1) was added LiOH (20 mg, 0.83 mmol) and the mixture was stirred at 60° C. overnight. The mixture was acidified with 1 N aq. HCl to pH˜5 and extracted with EtOAc twice. The combined organic layers were washed with brine, dried over anhydrous Na₂SO₄, filtered, and concentrated under reduced pressure to dryness to give the title compound (90 mg, yield 95.5%) as a yellow solid, which was used directly in the next step without further purification. LC/MS (ESI) m/z: 449 (M+H)⁺.

### Step 7: (R)—N-((1H-pyrrolo[3,2-c]pyridine-2-yl)methyl)-2-(3-((1-(dibenzo[b,d]furan-2-yl)ethyl)amino)-6-morpholino-2-oxopyrazin-1(2H)-yl)acetamide (Compound 407)

To a mixture of (R)-2-(3-((1-(dibenzo[b,d]furan-2-yl)ethyl)amino)-6-morpholino-2-oxopyrazin-1(2H)-yl)acetic acid (90 mg, 0.20 mmol) and (1H-pyrrolo[3,2-c]pyridine-2-yl)methanamine hydrochloride (55 mg, 0.13 mmol) in DMF (2 mL) was added DIPEA (103 mg, 0.80 mmol) and HATU (91 mg, 0.24 mmol) and the reaction mixture was stirred at room temperature for 0.5 hour. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-14% MeOH in DCM) and further purified by prep-HPLC to give Compound 407 (27.0 mg, yield 23.3%) as a white solid. ¹H NMR (400 MHz, DMSO-d₆) δ 11.28 (s, 1H), 8.78-8.70 (m, 2H), 8.15 (s, 1H), 8.12-8.09 (m, 2H), 7.69-7.66 (m, 1H), 7.63-7.57 (m, 2H), 7.50 (t, J=7.7 Hz, 1H), 7.39 (t, J=7.5 Hz, 1H), 7.35-7.30 (m, 2H), 6.66 (s, 1H), 6.43 (s, 1H), 5.28-5.19 (m, 1H), 4.75-4.65 (m, 2H), 4.45 (d, J=5.6 Hz, 2H), 3.68-3.48 (m, 2H), 3.41-3.32 (m, 2H), 2.81-2.63 (m, 4H), 1.58 (d, J=6.9 Hz, 3H). LC/MS (ESI) m/z: 578 (M+H)⁺. RT (Method A): 1.52 min.

### Scheme 125. Synthesis of N-(1-(2-(((1H-pyrrolo[3,2-c]pyridine-2-yl)methyl)amino)-2-oxoethyl)-2-morpholino-6-oxo-1,6-dihydropyrimidin-5-yl)-2-phenyloxazole-5-carboxamide (Compound 408)

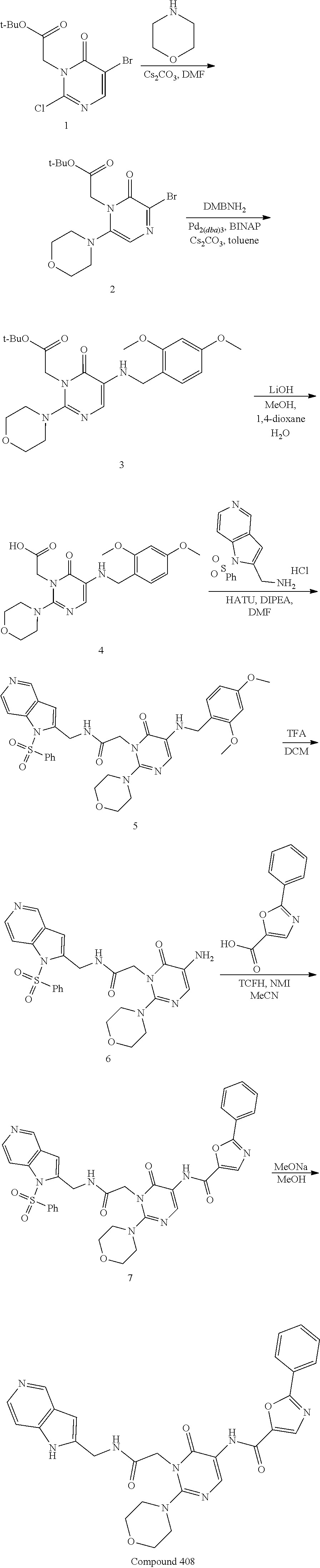

### Step 1: Tert-butyl 2-(5-bromo-2-morpholino-6-oxopyrimidin-1(6H)-yl)acetate (2)

To a mixture of tert-butyl 2-(5-bromo-2-chloro-6-oxopyrimidin-1(6H)-yl)acetate (6.0 g, 18.54 mmol) and morpholine (1.90 g, 21.81 mmol) in DMF (60 mL) was added Cs₂CO₃ (11.65 g, 35.76 mmol) under N₂ atmosphere. The mixture was degassed under N₂ atmosphere for three times and stirred at room temperature for 1 hour. The reaction mixture was diluted with EtOAc and filtered. The filtrate was washed with saturated water and brine, dried over anhydrous Na₂SO₄, filtered, and concentrated under reduced pressure to dryness. The residue was purified by flash chromatography (silica gel, 0-24% EtOAc in PE) to give the title compound (6.4 g, yield 92.2%) as a yellow solid. LC/MS (ESI) m/z: 374/376 (M+H)⁺.

### Step 2: Tert-butyl 2-(5-((2,4-dimethoxybenzyl)amino)-2-morpholino-6-oxopyrimidin-1(6H)-yl)acetate (3)

To a mixture of tert-butyl 2-(5-bromo-2-morpholino-6-oxopyrimidin-1(6H)-yl)acetate (4.0 g, 10.69 mmol) and (2,4-dimethoxyphenyl)methanamine (2.69 g, 16.10 mmol) in toluene (50 mL) was added Cs₂CO₃ (8.74 g, 26.80 mmol), BINAP (670 mg, 1.08 mmol) and Pd₂(dba)₃ (980 mg, 1.07 mmol) under N₂ atmosphere, the reaction mixture was degassed under N₂ atmosphere for three times and stirred at 110° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-2% MeOH in DCM) to give title compound (3.83 g, yield 77.8%) as red solid. LC/MS (ESI) m/z: 461 (M+H)⁺.

### Step 3: 2-(5-((2,4-Dimethoxybenzyl)amino)-2-morpholino-6-oxopyrimidin-1(6H)-yl)acetic acid (4)

To a solution of tert-butyl 2-(5-((2,4-dimethoxybenzyl)amino)-2-morpholino-6-oxopyrimidin-1(6H)-yl)acetate (2.0 g, 4.34 mmol) in MeOH/1,4-dioxane/water (25 mL, v/v/v=2/2/1) was added LiOH (533 mg, 22.21 mmol) and the mixture was stirred at 65° C. overnight. The mixture was acidified with 1 N aq. HCl to pH˜5 and extracted with EtOAc twice. The combined organic layers were washed with brine, dried over anhydrous Na₂SO₄, filtered, and concentrated under reduced pressure to dryness to give the title compound (1.70 g, yield 96.8%) as a yellow solid, which was used directly in the next step without further purification. LC/MS (ESI) m/z: 405 (M+H)⁺.

### Step 4: 2-(5-((2,4-Dimethoxybenzyl)amino)-2-morpholino-6-oxopyrimidin-1(6H)-yl)-N-((1-(phenylsulfonyl)-1H-pyrrolo[3,2-c]pyridine-2-yl)methyl)acetamide (5)

To a mixture of 2-(5-((2,4-dimethoxybenzyl)amino)-2-morpholino-6-oxopyrimidin-1(6H)-yl)acetic acid (1.70 g, 4.20 mmol) and (1-(phenylsulfonyl)-1H-pyrrolo[3,2-c]pyridine-2-yl)methanamine hydrochloride (2.18 g, 6.75 mmol) in DMF (20 mL) was added DIPEA (2.33 g, 18.06 mmol) and HATU (2.06 g, 5.41 mmol) and the reaction mixture was stirred at room temperature for 0.5 hour. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-6% MeOH in DCM) to give the title compound (1.85 g, yield 65.2%) as a yellow solid. LC/MS (ESI) m/z: 674 (M+H)⁺.

### Step 5: 2-(5-Amino-2-morpholino-6-oxopyrimidin-1(6H)-yl)-N-((1-(phenylsulfonyl)-1H-pyrrolo[3,2-c]pyridine-2-yl)methyl)acetamide (6)

To a mixture of 2-(5-((2,4-dimethoxybenzyl)amino)-2-morpholino-6-oxopyrimidin-1(6H)-yl)-N-((1-(phenylsulfonyl)-1H-pyrrolo[3,2-c]pyridine-2-yl)methyl)acetamide (1.44 g, 2.14 mmol) in DCM (10 mL) was added TFA (7 mL) and the reaction mixture was stirred at room temperature for 2 hours. The mixture was filtered, and the filter cake was washed with EtOAc three times, dried under vacuum to give title compound (1.30 g, yield 98.3%) as a white solid, which was used directly in the next step without further purification. LC/MS (ESI) (m/z): 524 (M+H)⁺.

### Step 6: N-(2-morpholino-6-oxo-1-(2-oxo-2-(((1-(phenylsulfonyl)-1H-pyrrolo[3,2-c]pyridine-2-yl)methyl)amino)ethyl)-1,6-dihydropyrimidin-5-yl)-2-phenyloxazole-5-carboxamide (7)

To a mixture of 2-(5-amino-2-morpholino-6-oxopyrimidin-1(6H)-yl)-N-((1-(phenylsulfonyl)-1H-pyrrolo[3,2-c]pyridine-2-yl)methyl)acetamide (60 mg, 0.11 mmol) and 2-phenyloxazole-5-carboxylic acid (21 mg, 0.11 mmol) in MeCN (3 mL) was added NMI (24 mg, 0.29 mmol) and TCFH (81 mg, 0.29 mmol) and the reaction mixture was stirred at room temperature for 0.5 hour. The mixture was diluted with DCM, washed with saturated aq. NH₄Cl solution and brine, dried over anhydrous Na₂SO₄, filtered, and concentrated under reduced pressure to dryness. The residue was purified by flash chromatography (silica gel, 0-4% MeOH in DCM) to give the title compound (60 mg, yield 75.4%) as a yellow oil. LC/MS (ESI) m/z: 695 (M+H)⁺.

### Step 7: N-(1-(2-(((1H-pyrrolo[3,2-c]pyridine-2-yl)methyl)amino)-2-oxoethyl)-2-morpholino-6-oxo-1,6-dihydropyrimidin-5-yl)-2-phenyloxazole-5-carboxamide (Compound 408)

To a solution of N-(2-morpholino-6-oxo-1-(2-oxo-2-(((1-(phenylsulfonyl)-1H-pyrrolo[3,2-c]pyridine-2-yl)methyl)amino)ethyl)-1,6-dihydropyrimidin-5-yl)-2-phenyloxazole-5-carboxamide (60 mg, 0.086 mmol) in MeOH (4 mL) was added MeONa (0.5 mL, 0.5 mmol, 1 M in MeOH) and the reaction mixture was stirred at room temperature for 3 hours. The mixture was diluted with EtOAc, washed with water and brine, dried over anhydrous Na₂SO₄, filtered, and concentrated under reduced pressure to dryness. The residue was purified by prep-HPLC to give Compound 408 (5.1 mg, yield 10.7%) as a white solid. ¹H NMR (400 MHz, CD₃OD) δ 8.76 (s, 1H), 8.70 (s, 1H), 8.23 (d, J=1.4 Hz, 1H), 8.21 (d, J=1.9 Hz, 1H), 8.11 (d, J=5.8 Hz, 1H), 8.05 (s, 1H), 7.65-7.63 (m, 2H), 7.62 (s, 1H), 7.44 (d, J=5.8 Hz, 1H), 6.63 (s, 1H), 4.87 (s, 2H), 4.65 (s, 2H), 3.76-3.74 (m, 4H), 3.22-3.19 (m, 4H). LC/MS (ESI) m/z: 555 (M+H)⁺. RT (Method A): 0.91 min.

### Scheme 126. Synthesis of N-((1H-pyrrolo[3,2-c]pyridine-2-yl)methyl)-2-(2-morpholino-6-oxo-5-((3-phenylpropyl)amino)pyrimidin-1(6H)-yl)acetamide (Compound 412)

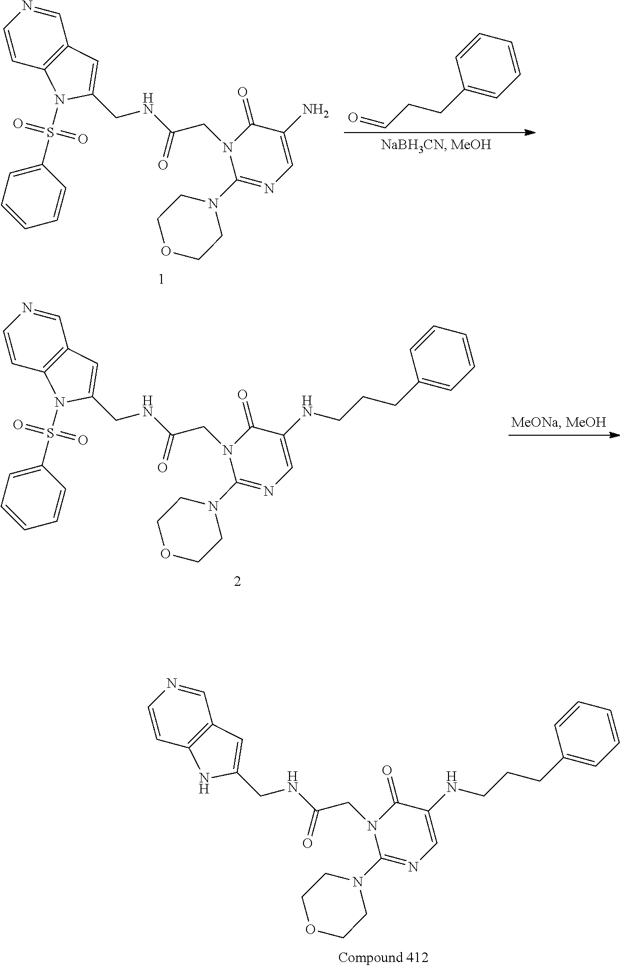

### Step 1: 2-(2-Morpholino-6-oxo-5-((3-phenylpropyl)amino)pyrimidin-1(6H)-yl)-N-((1-(phenylsulfonyl)-1H-pyrrolo[3,2-c]pyridine-2-yl)methyl)acetamide (2)

To a mixture of 2-(5-amino-2-morpholino-6-oxopyrimidin-1(6H)-yl)-N-((1-(phenylsulfonyl)-1H-pyrrolo[3,2-c]pyridine-2-yl)methyl)acetamide (150 mg, 28.7 mmol) and NaBH₃CN (54 mg, 86.0 mmol) in MeOH (2 mL) were added 3-phenylpropanal (19 mg, 14.3 mmol) under N₂ atmosphere. The mixture was degassed under N₂ atmosphere for three times and stirred at 50° C. for 1 hour. The mixture was quenched with saturated aq. NH₄Cl solution and extracted with EtOAc twice. The combined organic layers were dried over anhydrous Na₂SO₄, filtered, and concentrated under reduced pressure to dryness. The residue was purified by flash chromatography (silica gel, 0-10% MeOH in DCM) to give the title compound (43 mg, yield 23.4%) as a colorless oil. LC/MS (ESI) m/z: 642 (M+H)⁺.

### Step 2: N-((1H-pyrrolo[3,2-c]pyridine-2-yl)methyl)-2-(2-morpholino-6-oxo-5-((3-phenylpropyl)amino)pyrimidin-1(6H)-yl)acetamide (Compound 412)

To a solution of 2-(2-morpholino-6-oxo-5-((3-phenylpropyl)amino)pyrimidin-1(6H)-yl)-N-((1-(phenylsulfonyl)-1H-pyrrolo[3,2-c]pyridine-2-yl)methyl)acetamide (43 mg, 0.06 mmol) in MeOH (2 mL) was added NaOMe (1.34 mL, 0.67 mmol) at 0° C. and the mixture was stirred at room temperature for 1 hour. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness. The residue was purified by prep-TLC (DCM:MeOH=9:1) and further purified by prep-HPLC to give Compound 412 (4.8 mg, yield 14.3%) as a white solid. ¹H NMR (400 MHz, DMSO-d₆) δ 11.35 (s, 1H), 8.83 (s, 1H), 8.73 (s, 1H), 8.10 (d, J=5.6 Hz, 1H), 7.35-7.31 (m, 1H), 7.30-7.26 (m, 2H), 7.23-7.21 (m, 2H), 7.19-7.15 (m, 1H), 6.88 (s, 1H), 6.43 (s, 1H), 5.10 (t, J=5.8 Hz, 1H), 4.71 (s, 2H), 4.47 (d, J=4.7 Hz, 2H), 3.58-3.55 (m, 4H), 3.02 (d, J=6.5 Hz, 2H), 2.86-2.82 (m, 4H), 2.66-2.62 (m, 2H), 1.88-1.83 (m, 2H). LC/MS (ESI) (m/z): 502 (M+H)⁺. RT (Method A): 1.19 min.

### Scheme 127. Synthesis of N-(1-(2-(((1H-pyrrolo[3,2-c]pyridine-2-yl)methyl)amino)-2-oxoethyl)-2-methyl-6-oxo-1,6-dihydropyrimidin-5-yl)-2-phenyloxazole-4-carboxamide (Compound 414)

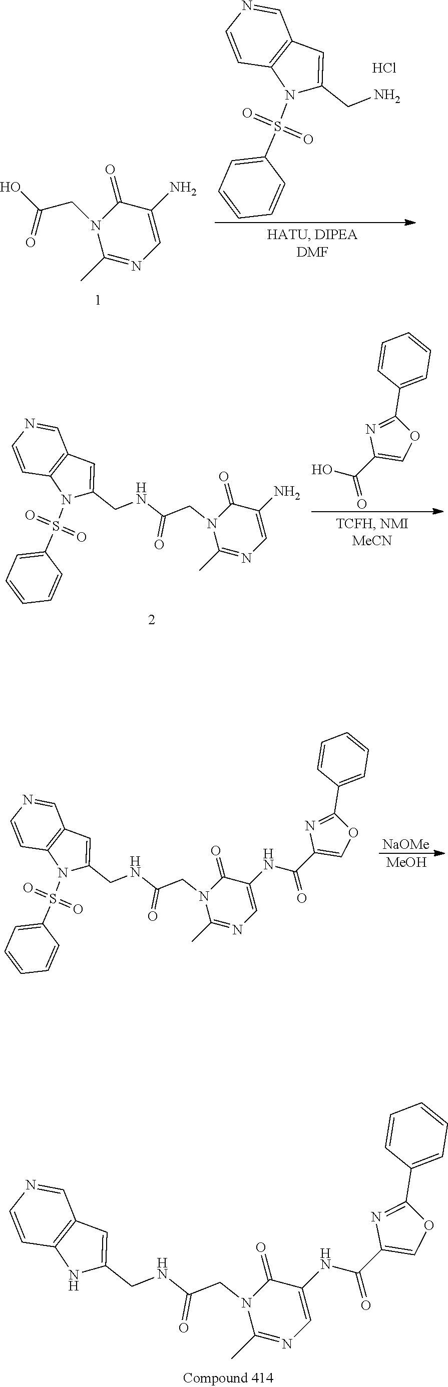

### Step 1: 2-(5-Amino-2-methyl-6-oxopyrimidin-1(6H)-yl)-N-((1-(phenylsulfonyl)-1H-pyrrolo[3,2-c]277yridine-2-yl)methyl)acetamide (2)

To a mixture of 2-(5-amino-2-methyl-6-oxopyrimidin-1(6H)-yl)acetic acid (150 mg, 0.81 mmol) and (1-(phenylsulfonyl)-1H-pyrrolo[3,2-c]pyridine-2-yl)methanamine hydrochloride (393 mg, 1.22 mmol) in DMF (3 mL) were added DIPEA (472 mg, 4.09 mmol) and HATU (463 mg, 1.22 mmol) and the mixture was stirred at room temperature for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-20% MeOH in DCM) to give the title compound (120 mg, yield 32.4%) as a yellow solid. LC/MS (ESI) (m/z): 453 (M+H)⁺.

### Step 2: N-(1-(2-(((1H-pyrrolo[3,2-c]pyridine-2-yl)methyl)amino)-2-oxoethyl)-2-methyl-6-oxo-1,6-dihydropyrimidin-5-yl)-2-phenyloxazole-4-carboxamide (Compound 414)

To a mixture of 2-(5-amino-2-methyl-6-oxopyrimidin-1(6H)-yl)-N-((1-(phenylsulfonyl)-1H-pyrrolo[3,2-c]pyridine-2-yl)methyl)acetamide (100 mg, 0.22 mmol) and 2-phenyloxazole-4-carboxylic acid (43 mg, 0.23 mmol) in DMF (3 mL) were added NMI (43 mg, 53 mmol) and TCFH (148 mg, 0.53 mmol). The mixture was stirred at room temperature for 18 hours. The mixture was diluted with EtOAc, washed with water and brine, dried over anhydrous Na₂SO₄, filtered, and concentrated under reduced pressure to dryness. The residue was dissolved in MeOH (1 mL) and MeONa (49 mg, 0.92 mmol) was added, and the mixture was stirred at room temperature for 1 hour. The mixture was neutralized with 1N aq. HCl at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) and further purified by prep-HPLC to give Compound 414 (4.5 mg, yield 4.21%) as a white solid. ¹H NMR (400 MHz, DMSO-d₆) δ 12.51 (s, 1H), 9.35 (s, 1H), 9.13 (s, 1H), 9.09 (t, J=5.8 Hz, 1H), 8.98 (s, 1H), 8.78 (s, 1H), 8.33 (d, J=6.4 Hz, 1H), 8.07-8.04 (m, 2H), 7.83 (d, J=6.5 Hz, 1H), 7.62 (d, J=1.4 Hz, 2H), 7.60 (s, 1H), 6.83 (s, 1H), 4.89 (s, 2H), 4.60 (d, J=5.5 Hz, 2H), 2.48 (s, 3H). LC/MS (ESI) (m/z): 484 (M+H)⁺. RT (Method A): 1.01 min.

### Scheme 128. Synthesis of (R)—N-((1H-Pyrrolo[3,2-c]pyridine-2-yl)methyl)-2-(3-((1-(2′-fluoro-[1,1′-biphenyl]-4-yl)ethyl)amino)-6-(2-fluorophenyl)-2-oxopyrazin-1(2H)-yl) acetamide (Compound 418)

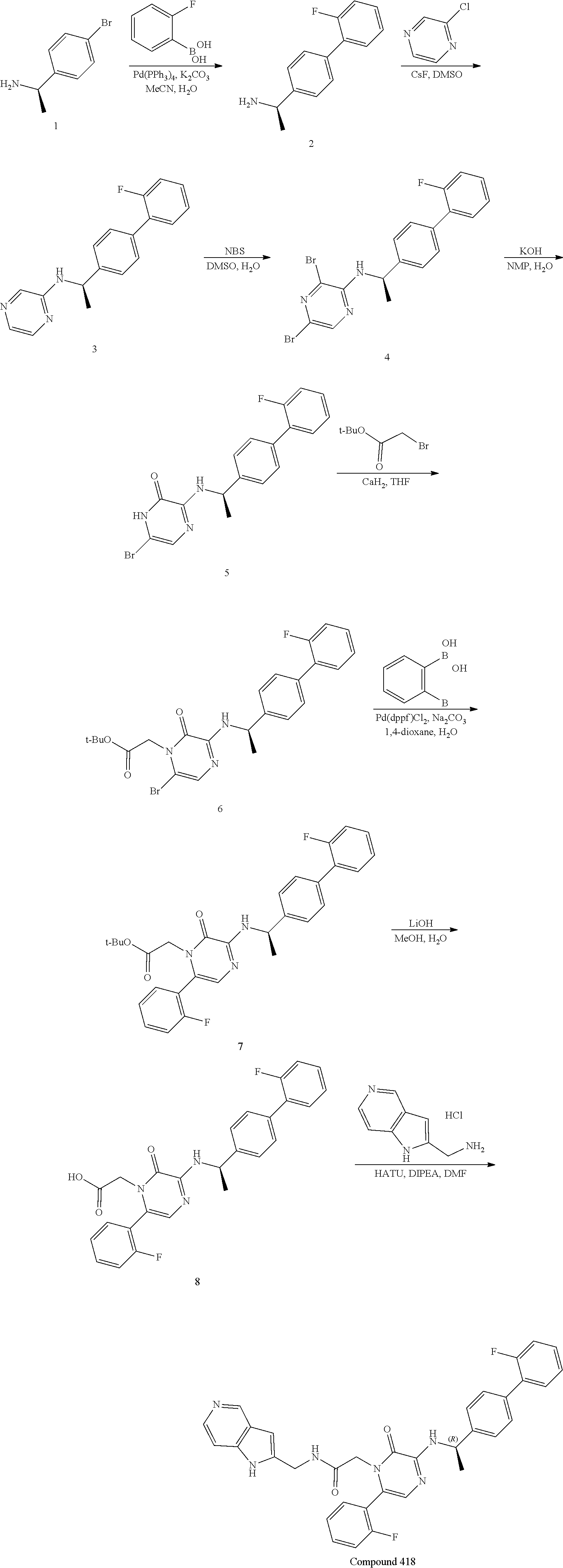

### Step 1: (R)-1-(2′-Fluoro-[1,1′-biphenyl]-4-yl)ethan-1-amine (2)

To a mixture of (R)-1-(4-bromophenyl)ethan-1-amine (10 g, 0.05 mol) and (2-fluorophenyl)boronic acid (9.69 g, 0.065 mol) in MeCN (100 mL) and water (33 mL) was added K₂CO₃ (13.8 g, 0.1 mol) and Pd(PPh₃)₄ (5.77 g, 0.005 mol) under N₂ atmosphere, the reaction mixture was degassed under N₂ atmosphere for three times and stirred at 80° C. for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-5% DCM in MeOH) to give the title compound (9.6 g, yield 88.8%) as a yellow oil. LC/MS (ESI) m/z: 199 (M+H)⁺.

### Step 2: (R)—N-(1-(2′-Fluoro-[1,1′-biphenyl]-4-yl)ethyl)pyrazin-2-amine (3)

To a solution of (R)-1-(2′-fluoro-[1,1′-biphenyl]-4-yl)ethan-1-amine (6.2 g, 0.029 mol) and 2-chloropyrazine (6.61 g, 0.058 mol) in DMSO (60 mL) was added CsF (13.22 mg, 0.087 mol) and the reaction mixture was stirred at 100° C. for 16 hours. The mixture was diluted with EtOAc, washed with water and brine, dried over anhydrous Na₂SO₄, filtered, and concentrated under reduced pressure to dryness. The residue was purified by flash chromatography (silica gel, 10-30% EtOAc in PE) to give the title compound (6.4 g, yield 75.7%) as a yellow oil. LC/MS (ESI) m/z: 294 (M+H)⁺.

### Step 3: (R)-3,5-Dibromo-N-(1-(2′-fluoro-[1,1′-biphenyl]-4-yl)ethyl)pyrazin-2-amine (4)

To a mixture of (R)—N-(1-(2′-fluoro-[1,1′-biphenyl]-4-yl)ethyl)pyrazin-2-amine (6.0 g, 0.02 mol) in DMSO/water (62.4 mL, v/v=25/1) was added NBS (10.8 g, 0.06 mmol) at 0° C. and the reaction mixture was stirred at 50° C. for 16 hours. The mixture was diluted with EtOAc, washed with water and brine, dried over anhydrous Na₂SO₄, filtered, and concentrated under reduced pressure to dryness. The residue was purified by flash chromatography (silica gel, 10-30% EtOAc in PE) to give the title compound (5.1 g, yield 55.5%) as a yellow oil. LC/MS (ESI) m/z: 452 (M+H)⁺.

### Step 4: (R)-6-Bromo-3-((1-(2′-fluoro-[1,1′-biphenyl]-4-yl)ethyl)amino)pyrazin-2(1H)-one (5)

To a mixture of (R)-3,5-dibromo-N-(1-(2′-fluoro-[1,1′-biphenyl]-4-yl)ethyl)pyrazin-2-amine (5 g, 0.011 mol) and KOH (6.25 g, 0.11 mmol) in NMP/water (66.7 mL, v/v=3/1), the reaction mixture was stirred at 100° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 10-30% EtOAc in PE) to give the title compound (3.5 g, yield 81.4%) as a yellow oil. LC/MS (ESI) m/z: 388 (M+H)⁺.

### Step 5: Tert-Butyl (R)-2-(6-bromo-3-((1-(2′-fluoro-[1,1′-biphenyl]-4-yl)ethyl)amino)-2-oxopyrazin-1(2H)-yl)acetate (6)

To a mixture of (R)-6-bromo-3-((1-(2′-fluoro-[1,1′-biphenyl]-4-yl)ethyl)amino)pyrazin-2(1H)-one (2.5 g, 0.006 mol) and tert-butyl 2-bromoacetate (2.46 g, 0.012 mol) in THF (25 mL) was added CaH₂ (757.6 mg, 0.018 mol), the reaction mixture was stirred at 60° C. for 2 hours. The reaction mixture was quenched by the addition of saturated aq. NH₄Cl solution (200 mL) and extracted with EtOAc twice. The combined organic layers were washed with water and brine, dried over anhydrous Na₂SO₄, filtered, and concentrated under reduced pressure to dryness. The residue was purified by flash chromatography (silica gel, 10-30% EtOAc in PE) to give the title compound (1 g, yield 30.9%) as a yellow solid. LC/MS (ESI) m/z: 502 (M+H)⁺.

### Step 6: Tert-Butyl (R)-2-(3-((1-(2′-fluoro-[1,1′-biphenyl]-4-yl)ethyl)amino)-6-(2-fluorophenyl)-2-oxopyrazin-1(2H)-yl)acetate (7)

To a mixture of tert-butyl (R)-2-(6-bromo-3-((1-(2′-fluoro-[1,1′-biphenyl]-4-yl)ethyl)amino)-2-oxopyrazin-1(2H)-yl)acetate (70 mg, 0.14 mmol) and (2-fluorophenyl)boronic acid (39 mg, 0.28 mmol) in 1,4-dioxane/water (2.5 mL, v/v=5/1) was added Na₂CO₃ (44.52 mg, 0.42 mmol) and Pd(dppf)Cl₂ (11.5 mg, 0.014 mmol) under N₂ atmosphere, the reaction mixture was degassed under N₂ atmosphere for three times and stirred at 100° C. overnight. The mixture was diluted with water and extracted with EtOAc twice. The combined organic layers were washed with water and brine, dried over anhydrous Na₂SO₄, filtered, and concentrated under reduced pressure to dryness. The residue was purified by flash chromatography (silica gel, 10-30% EtOAc in PE) to give the title compound (40 mg, yield 55.4%) as a yellow solid. LC/MS (ESI) m/z: 518 (M+H)⁺.

### Step 7: (R)-2-(3-((1-(2′-Fluoro-[1,1′-biphenyl]-4-yl)ethyl)amino)-6-(2-fluorophenyl)-2-oxopyrazin-1(2H)-yl)acetic acid (8)

To a solution of tert-butyl (R)-2-(3-((1-(2′-fluoro-[1,1′-biphenyl]-4-yl)ethyl)amino)-6-(2-fluorophenyl)-2-oxopyrazin-1(2H)-yl)acetate (40 mg, 0.08 mmol) in MeOH/water (2.5 mL, v/v=4/1) was added LiOH·H₂O (18.5 mg, 0.80 mmol) and the mixture was stirred at 60° C. for 16 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (40 mg, yield 100%) as a yellow solid, which was used directly in the next step without further purification. LC/MS (ESI) m/z: 462 (M+H)⁺.

### Step 8: (R)—N-((1H-Pyrrolo[3,2-c]pyridine-2-yl)methyl)-2-(3-((1-(2′-fluoro-[1,1′-biphenyl]-4-yl)ethyl)amino)-6-(2-fluorophenyl)-2-oxopyrazin-1(2H)-yl)acetamide (Compound 418)

To a mixture of I-2-(3-((1-(2′-fluoro-[1,1′-biphenyl]-4-yl)ethyl)amino)-6-(2-fluorophenyl)-2-oxopyrazin-1(2H)-yl)acetic acid (40 mg, 0.11 mmol) and (1H-pyrrolo[3,2-c]pyridine-2-yl)methanamine hydrochloride (32 mg, 0.22 mmol) in DMF (2 mL) was added DIPEA (70.1 mg, 0.55 mmol) and HATU (63 mg, 0.17 mmol) and the reaction mixture was stirred at room temperature for 1 hour. The mixture was concentrated under reduced pressure to dryness. The residue was purified by prep-HPLC to give Compound 418 (10 mg, yield 15.4%) as a white solid. ¹H NMR (400 MHz, DMSO-d₆) δ 11.25 (s, 1H), 8.73 (s, 1H), 8.54 (t, J=5.5 Hz, 1H), 8.11 (d, J=5.7 Hz, 1H), 7.75 (d, J=8.3 Hz, 1H), 7.55-7.48 (m, 6H), 7.43-7.38 (m, 1H), 7.34-7.27 (m, 5H), 7.14 (t, J=7.4 Hz, 1H), 6.70-6.66 (m, 1H), 6.20 (s, 1H), 5.25-5.17 (m, 1H), 4.95-4.60 (m, 1H), 4.32 (d, J=5.3 Hz, 2H), 4.17-3.88 (m, 1H), 1.57 (d, J=7.0 Hz, 3H). LC/MS (ESI) m/z: 591 (M+H)⁺.

### Scheme 129. Synthesis of (R)—N-((1H-Pyrrolo[3,2-c]pyridine-2-yl)methyl)-2-(3-((1-(dibenzo[b,d]furan-2-yl)ethyl)amino)-6-(3,6-dihydro-2H-pyran-4-yl)-2-oxopyrazin-1(2H)-yl)acetamide (Compound 426)

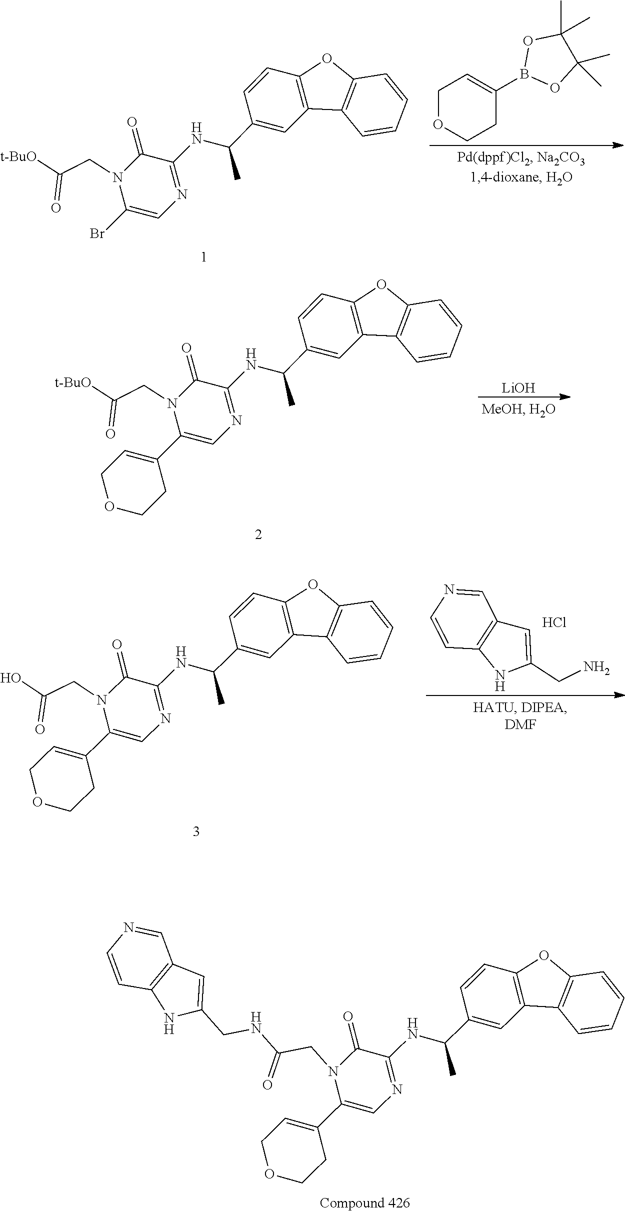

### Step 1: Tert-Butyl (R)-2-(3-((1-(dibenzo[b,d]furan-2-yl)ethyl)amino)-6-(3,6-dihydro-2H-pyran-4-yl)-2-oxopyrazin-1(2H)-yl)acetate (2)

To a mixture of tert-butyl (R)-2-(6-bromo-3-((1-(dibenzo[b,d]furan-2-yl)ethyl)amino)-2-oxopyrazin-1(2H)-yl)acetate (120 mg, 0.24 mmol) and 2-(3,6-dihydro-2H-pyran-4-yl)-4,4,5,5-tetramethyl-1,3,2-dioxaborolane (101 mg, 0.48 mmol) in 1,4-dioxane/water (3.5 mL, v/v=6/1) was added Na₂CO₃ (74 mg, 0.70 mmol), Pd(dppf)Cl₂ (18 mg, 0.025 mmol) under N₂ atmosphere, the reaction mixture was degassed under N₂ atmosphere for three times and stirred at 105° C. for 6 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-36% EtOAc in PE) to give title compound (82 mg, yield 67.9%) as a yellow oil. LC/MS (ESI) m/z: 502 (M+H)⁺.

### Step 2: (R)-2-(3-((1-(Dibenzo[b,d]furan-2-yl)ethyl)amino)-6-(3,6-dihydro-2H-pyran-4-yl)-2-oxopyrazin-1(2H)-yl)acetic acid (3)

To a solution of tert-butyl (R)-2-(3-((1-(dibenzo[b,d]furan-2-yl)ethyl)amino)-6-(3,6-dihydro-2H-pyran-4-yl)-2-oxopyrazin-1(2H)-yl)acetate (80 mg, 0.16 mmol) in MeOH/1,4-dioxane/water (25 mL, v/v/v=2/2/1) was added LiOH (15 mg, 0.63 mmol) and the mixture was stirred at 60° C. overnight. The mixture was acidified with 1 N aq. HCl to pH˜5 and extracted with EtOAc twice. The combined organic layers were washed with brine, dried over anhydrous Na₂SO₄, filtered, and concentrated under reduced pressure to dryness to give the title compound (70 mg, yield 96.8%) as a white solid, which was used directly in the next step without further purification. LC/MS (ESI) m/z: 446 (M+H)⁺.

### Step 3: (R)—N-((1H-Pyrrolo[3,2-c]pyridine-2-yl)methyl)-2-(3-((1-(dibenzo[b,d]furan-2-yl)ethyl)amino)-6-(3,6-dihydro-2H-pyran-4-yl)-2-oxopyrazin-1(2H)-yl)acetamide (Compound 426)

To a mixture of (R)-2-(3-((1-(dibenzo[b,d]furan-2-yl)ethyl)amino)-6-(3,6-dihydro-2H-pyran-4-yl)-2-oxopyrazin-1(2H)-yl)acetic acid (50 mg, 0.11 mmol) and (1H-pyrrolo[3,2-c]pyridine-2-yl)methanamine hydrochloride (30 mg, 0.16 mmol) in DMF (2 mL) was added DIPEA (60 mg, 0.47 mmol) and HATU (50 mg, 0.13 mmol) and the reaction mixture was stirred at room temperature for 0.5 hour. The mixture was diluted with EtOAc, washed with saturated aq. NH₄Cl solution and brine, dried over anhydrous Na₂SO₄, filtered, and concentrated under reduced pressure to dryness. The residue was purified by prep-HPLC to give Compound 426 (27 mg, yield 41.9%) as a white solid. ¹H NMR (400 MHz, DMSO-d₆) δ 11.29 (s, 1H), 8.77-8.68 (m, 2H), 8.17 (s, 1H), 8.11 (d, J=5.8 Hz, 2H), 7.68 (d, J=8.2 Hz, 1H), 7.64-7.58 (m, 2H), 7.53-7.48 (m, 2H), 7.42-7.36 (m, 1H), 7.32 (d, J=5.6 Hz, 1H), 6.57 (s, 1H), 6.41 (s, 1H), 5.81 (s, 1H), 5.31-5.23 (m, 1H), 4.57-4.46 (m, 2H), 4.44 (d, J=5.7 Hz, 2H), 3.99 (d, J=2.4 Hz, 2H), 3.63 (t, J=5.3 Hz, 2H), 2.14-2.06 (m, 2H), 1.59 (d, J=7.0 Hz, 3H). LC/MS (ESI) m/z: 575 (M+H)⁺. RT (Method A): 1.57 min.

Compound 427 was prepared based on Scheme 129:

| # | Reactant A | Reactant B | Reactant C | Characterization Data |
|---|---|---|---|---|
| 427^{a} | 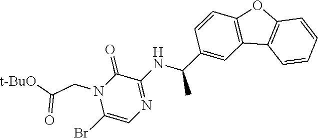 | 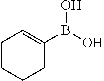 | 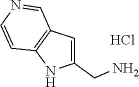 [SMILES: NCc1cc2cnccc2[nH]1 ; Cl] | ¹H NMR (400 MHz, DMSO-d₆) δ 11.29 (s, 1H), 8.75-8.66 (m, 2H), 8.16 (s, 1H), 8.10 (d, J = 7.1 Hz, 2H), 7.67 (d, J = 8.1 Hz, 1H), 7.61 (t, J = 6.0 Hz, 2H), 7.50 (t, J = 7.7 Hz, 1H), 7.43-7.36 (m, 2H), 7.32 (d, J = 5.2 Hz, 1H), 6.49 (s, 1H), 6.40 (s, 1H), 5.72 (s, 1H), 5.30-5.19 (m, 1H), 4.53-4.42 (m, 4H), 2.02-1.93 (m, 4H), 1.58 (d, J = 6.7 Hz, 3H), 1.56-1.46 (m, 4H). LC/MS (ESI) m/z: 573 (M + H)⁺. RT (Method A): 1.89 min. |

^{a}Step 1 was performed in the presence of Pd(PPh₃)₄ and K₂CO₃ in 1,4-dioxane/H₂O.

### Scheme 130. Synthesis of (S)—N-((1H-Pyrrolo[3,2-c]pyridine-2-yl)methyl)-2-(5-((1-(dibenzo[b,d]furan-2-yl)-2-hydroxyethyl)amino)-2-(2-fluorophenyl)-6-oxopyrimidin-1(6H)-yl)acetamide (Compound 433)

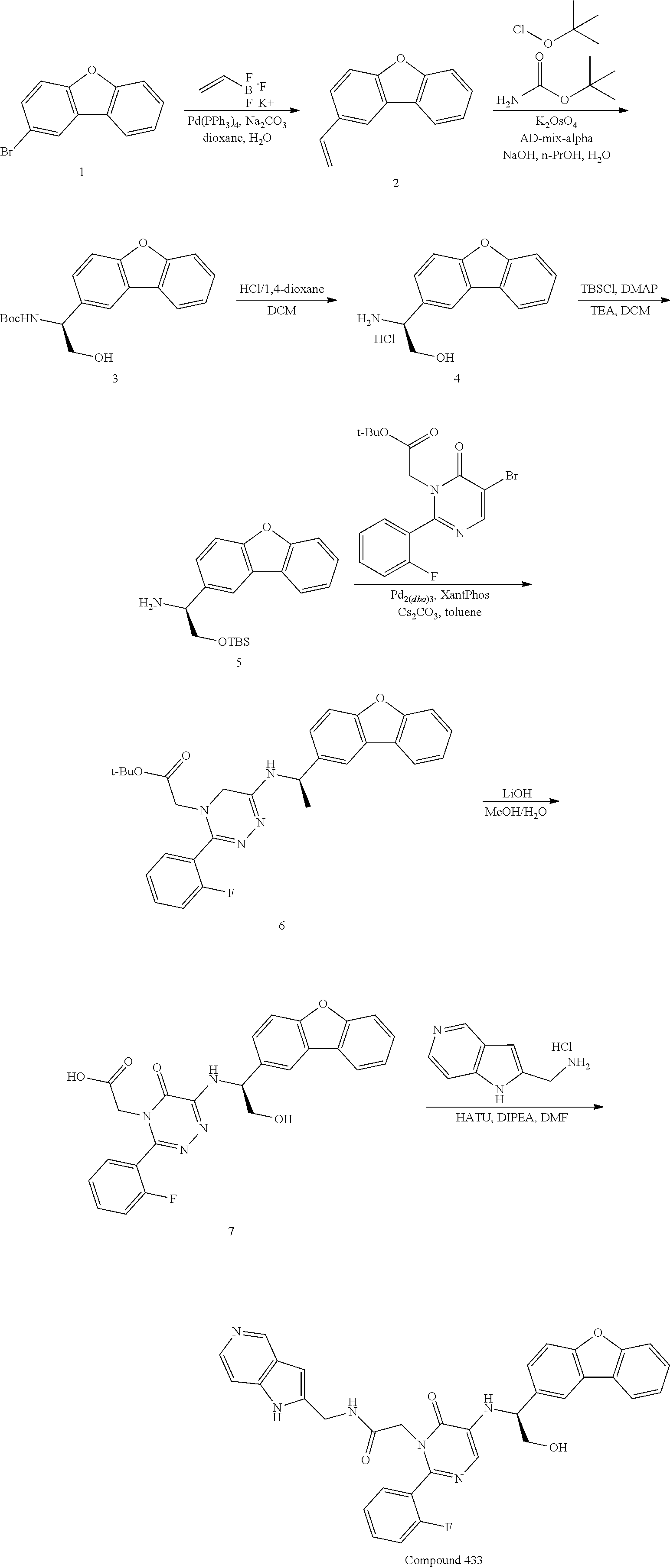

### Step 1: 2-Vinyldibenzo[b,d]furan (2)

To a mixture of 2-bromodibenzo[b,d]furan (10 g, 40.47 mmol), potassium trifluoro(vinyl)borate (10.84 g, 80.93 mmol), Na₂CO₃ (12.87 g, 121.43 mmol) in 1,4-dioxane (100 mL) and water (10 mL) was added Pd(PPh₃)₄ (2.34 g, 2.02 mmol) under N₂ atmosphere. The mixture was degassed under N₂ atmosphere for three times and stirred under N₂ atmosphere at 80° C. for 16 hours. The mixture was diluted with EtOAc, washed with water and brine, dried over anhydrous Na₂SO₄, filtered, and concentrated to dryness. The residue was purified by flash chromatography (silica gel, 0-1% EtOAc in PE) to give the title compound (4.8 g, 61.1%) as a white solid. LC/MS (ESI) m/z: 195 (M+H)⁺.

### Step 2: Tert-Butyl (S)-(1-(dibenzo[b,d]furan-2-yl)-2-hydroxyethyl)carbamate (3)

To a mixture of tert-butyl hypochlorite (5.48 g, 50.47 mmol) and tert-butyl carbamate (5.91 g, 50.45 mmol) in n-PrOH (30 mL) was added a solution of NaOH (2.02 g, 50.50 mmol) in water (60 mL) dropwise at 0° C. The mixture was stirred at room temperature for 5 minutes. The reaction mixture was cooled to 0° C. and a solution of AD-mix-alpha (674 mg, 0.87 mmol) in n-PrOH (30 mL) was added. The mixture was stirred at 0° C. for another 5 minutes. To the mixture was added a solution of 2-vinyldibenzo[b,d]furan (2.8 g, 14.42 mmol) in n-PrOH (60 mL) dropwise followed by K₂OsO₄·2H₂O (212 mg, 0.58 mmol) at 0° C. The resulting mixture was stirred at 0° C. for 3 hours. The reaction mixture was quenched with saturated aq. Na₂S₂O₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-33% EtOAc in PE) to give the title compound (1.5 g, yield 31.8%) as a white solid. LC/MS (ESI) m/z: 328 (M+H)⁺.

### Step 3: (S)-2-Amino-2-(dibenzo[b,d]furan-2-yl)ethan-1-ol hydrochloride (4)

To a solution of tert-butyl (S)-(1-(dibenzo[b,d]furan-2-yl)-2-hydroxyethyl)carbamate (600 mg, 1.83 mmol) in DCM (4 mL) was added HCl/1,4-dioxane (4 mL, 4M) and the mixture was stirred at room temperature for 0.5 hour. The mixture was concentrated under reduced pressure to dryness to give the title compound (480 mg, yield 99%) as a yellow solid, which was used directly in the next step without further purification. LC/MS (ESI) m/z: 228 (M+H)⁺.

### Step 4: (S)-2-((tert-Butyldimethylsilyl)oxy)-1-(dibenzo[b,d]furan-2-yl)ethan-1-amine (5)

To a solution of (S)-2-amino-2-(dibenzo[b,d]furan-2-yl)ethan-1-ol hydrochloride (480 mg, 1.82 mmol) in DCM (10 mL) was added TEA (356 mg, 3.52 mmol) and DMAP (43 mg, 0.35 mmol) followed by TBSCl (318 mg, 2.11 mmol) at 0° C. and the mixture was stirred at room temperature for 12 hours. The mixture was quenched with water and extracted with DCM twice. The combined organic layers were washed with brine, dried over anhydrous Na₂SO₄, filtered, and concentrated under reduced pressure to dryness. The residue was purified by flash chromatography (silica gel, 0-7% MeOH in DCM) to give the title compound (510 mg, yield 82%) as a yellow oil. LC/MS (ESI) m/z: 342 (M+H)⁺.

### Step 5: Tert-Butyl (S)-2-(5-((2-((tert-butyldimethylsilyl)oxy)-1-(dibenzo[b,d]furan-2-yl)ethyl)amino)-2-(2-fluorophenyl)-6-oxopyrimidin-1(6H)-yl)acetate (6)

To a mixture of (S)-2-((tert-butyldimethylsilyl)oxy)-1-(dibenzo[b,d]furan-2-yl)ethan-1-amine (510 mg, 1.49 mmol) and tert-butyl 2-(5-bromo-2-(2-fluorophenyl)-6-oxopyrimidin-1(6H)-yl)acetate (687 mg, 1.79 mmol), in toluene (60 mL) was added Cs₂CO₃ (1.46 g, 4.48 mmol) and XantPhos (172 mg, 0.30 mmol) followed by Pd₂(dba)₃ (137 mg, 0.15 mmol) under N₂ atmosphere. The mixture was degassed under N₂ atmosphere for three times and stirred under N₂ atmosphere at 120° C. for 16 hours. The mixture was diluted with EtOAc, washed with water and brine, dried over anhydrous Na₂SO₄, filtered, and concentrated to dryness. The residue was purified by flash chromatography (silica gel, 0-13% EtOAc in PE) to give the title compound (460 mg, yield 47.8%) as a yellow solid. LC/MS (ESI) m/z: 644 (M+H)⁺.

### Step 6: (S)-2-(5-((1-(Dibenzo[b,d]furan-2-yl)-2-hydroxyethyl)amino)-2-(2-fluorophenyl)-6-oxopyrimidin-1(6H)-yl)acetic acid (7)

To a solution of tert-butyl (S)-2-(5-((2-((tert-butyldimethylsilyl)oxy)-1-(dibenzo[b,d]furan-2-yl)ethyl)amino)-2-(2-fluorophenyl)-6-oxopyrimidin-1(6H)-yl)acetate (460 mg, 0.71 mmol) in MeOH/water (6 mL, v/v=3/1) was added LiOH (120 mg, 2.86 mmol) and the mixture was stirred at 50° C. for 12 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (320 mg, yield 94.6%) as a yellow solid, which was used directly in the next step without further purification. LC/MS (ESI) m/z: 474 (M+H)⁺.

### Step 7: (S)—N-((1H-Pyrrolo[3,2-c]pyridine-2-yl)methyl)-2-(5-((1-(dibenzo[b,d]furan-2-yl)-2-hydroxyethyl)amino)-2-(2-fluorophenyl)-6-oxopyrimidin-1(6H)-yl)acetamide (Compound 433)

To a mixture of (S)-2-(5-((1-(dibenzo[b,d]furan-2-yl)-2-hydroxyethyl)amino)-2-(2-fluorophenyl)-6-oxopyrimidin-1(6H)-yl)acetic acid (50 mg, 0.11 mmol) and (1H-pyrrolo[3,2-c]pyridine-2-yl)methanamine hydrochloride (23 mg, 0.13 mmol) in DMF (2 mL) was added DIPEA (68 mg, 0.53 mmol) and HATU (60 mg, 0.16 mmol) and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 433 (5.7 mg, yield 9.0%) as a white solid. ¹H NMR (400 MHz, DMSO-d₆) δ 11.27 (s, 1H), 8.73 (s, 1H), 8.60 (t, J=5.6 Hz, 1H), 8.20 (d, J=1.4 Hz, 1H), 8.12 (t, J=6.8 Hz, 2H), 7.70-7.67 (m, 2H), 7.61-7.58 (m, 1H), 7.53-7.47 (m, 2H), 7.39 (t, J=7.5 Hz, 1H), 7.35-7.26 (m, 3H), 7.14 (t, J=7.5 Hz, 1H), 6.91 (s, 1H), 6.23 (s, 1H), 6.01 (d, J=5.3 Hz, 1H), 5.30 (t, J=5.6 Hz, 1H), 4.94-4.57 (m, 1H), 4.56-4.52 (m, 1H), 4.34 (d, J=5.5 Hz, 2H), 4.33-4.00 (m, 1H), 3.81-3.69 (m, 2H). LC/MS (ESI) (m/z): 603 (M+H)⁺. RT (Method A): 1.43 min.

### Scheme 131. Synthesis of (R)—N-((1H-Pyrrolo[3,2-c]pyridine-2-yl)methyl)-2-(6-oxo-5-((1-(5-phenylthiophen-2-yl)ethyl)amino)-2-(piperidin-1-yl)pyrimidin-1(6H)-yl)acetamide (Compound 445)

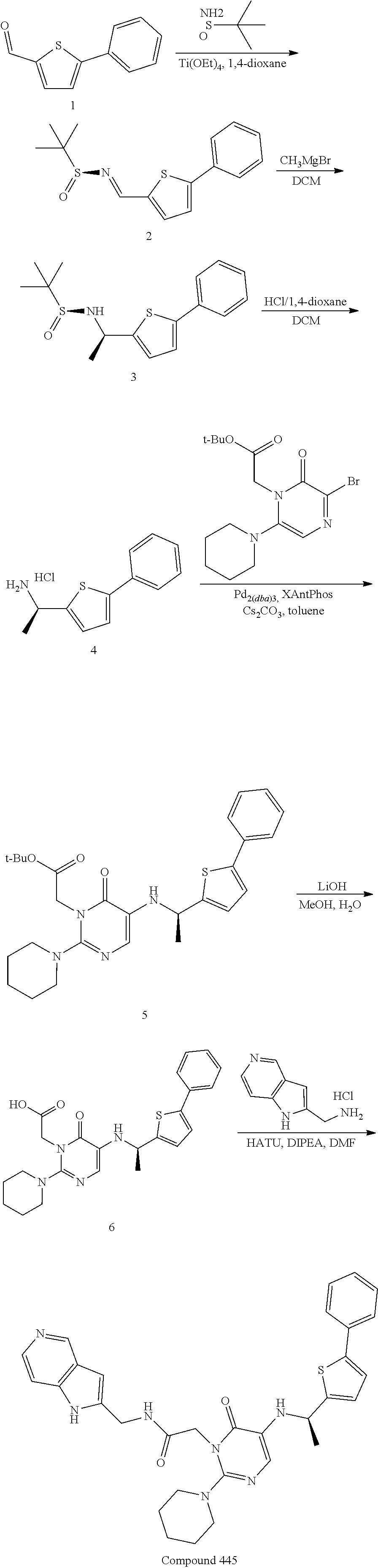

### Step 1: (S,E)-2-Methyl-N-((5-phenylthiophen-2-yl)methylene)propane-2-sulfinamide (2)

To a mixture of 5-phenylthiophene-2-carbaldehyde (1.00 g, 5.31 mmol) and (S)-2-methylpropane-2-sulfinamide (2.57 g, 21.2 mmol) in 1,4-dioxane (10 mL) was added Ti(OEt)₄ (4.85 g, 21.2 mmol), the reaction mixture was degassed under N₂ atmosphere for three times and stirred at 90° C. for 2 hours. The mixture was quenched with ice-water and extracted with EtOAc twice. The combined organic layers were washed with brine, dried over anhydrous Na₂SO₄, filtered, and concentrated to give title compound (1.4 g, yield 93.3%) as a yellow solid. LC/MS (ESI) (m/z): 292 (M+H)⁺.

### Step 2: (S)-2-Methyl-N—((R)-1-(5-phenylthiophen-2-yl)ethyl)propane-2-sulfinamide (3)

To a solution of (S,E)-2-methyl-N-((5-phenylthiophen-2-yl)methylene)propane-2-sulfinamide (1.2 g, 4.11 mmol) in DCM (10 mL) was added MeMgBr (4.1 mL, 4.1 mmol, 1.0 M in THF) dropwise at −78° C. under N₂ atmosphere and the mixture was stirred at −78° C. for 2 hours. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give title compound (700 mg, yield 58.3%) as a white solid. LC/MS (ESI) (m/z): 308 (M+H)⁺.

### Step 3: (R)-1-(5-Phenylthiophen-2-yl)ethan-1-amine hydrochloride (4)

To a solution of (S)-2-methyl-N—((R)-1-(5-phenylthiophen-2-yl)ethyl)propane-2-sulfinamide (700 mg, 2.28 mmol) in DCM (5.0 mL) was added HCl/1,4-dioxane (5 mL, 4M) and the mixture was stirred at room temperature for 0.5 hour. The mixture was filtered, and the filter cake was washed with EtOAc, dried under vacuum to give title compound (425 mg, yield 86.1%) as a white solid. LC/MS (ESI) (m/z): 204 (M+H)⁺.

### Step 4: Tert-Butyl (R)-2-(6-oxo-5-((1-(5-phenylthiophen-2-yl)ethyl)amino)-2-(piperidin-1-yl)pyrimidin-1(6H)-yl)acetate (5)

To a mixture of (R)-1-(5-phenylthiophen-2-yl)ethan-1-amine (115 mg, 0.48 mmol) and tert-butyl 2-(5-bromo-6-oxo-2-(piperidin-1-yl)pyrimidin-1(6H)-yl)acetate (120 mg, 0.32 mmol) in toluene (5 mL) was added Cs₂CO₃ (315 g, 0.96 mmol), XantPhos (19 mg, 0.03 mmol) and Pd₂(dba)₃ (30 mg, 0.03 mmol), the reaction mixture was degassed under N₂ atmosphere for three times and stirred at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give title compound (70 mg, yield 44.0%) as a yellow solid. LC/MS (ESI) m/z: 495 (M+H)⁺.

### Step 5: (R)-2-(6-Oxo-5-((1-(5-phenylthiophen-2-yl)ethyl)amino)-2-(piperidin-1-yl)pyrimidin-1(6H)-yl)acetic acid (6)

To a mixture of tert-butyl (R)-2-(6-oxo-5-((1-(5-phenylthiophen-2-yl)ethyl)amino)-2-(piperidin-1-yl)pyrimidin-1(6H)-yl)acetate (70 mg, 0.14 mmol) in MeOH (3.0 mL) and water (1.0 mL) was added LiOH (7.0 mg, 0.28 mmol) and the mixture was stirred at 50° C. for 16 hours. The mixture was acidified with 1N aq. HCl to pH˜3 and extracted with EtOAc twice. The combined organic layers were washed with brine, dried over anhydrous Na₂SO₄, filtered, and concentrated to give the title compound (58 mg, yield 93%) as a yellow solid. LC/MS (ESI) (m/z): 439 (M+H)⁺.

### Step 6: (R)—N-((1H-Pyrrolo[3,2-c]pyridine-2-yl)methyl)-2-(6-oxo-5-((1-(5-phenylthiophen-2-yl)ethyl)amino)-2-(piperidin-1-yl)pyrimidin-1(6H)-yl)acetamide (Compound 445)

To a mixture of (R)-2-(6-oxo-5-((1-(5-phenylthiophen-2-yl)ethyl)amino)-2-(piperidin-1-yl)pyrimidin-1(6H)-yl)acetic acid (58 mg, 0.13 mmol) and (1H-pyrrolo[3,2-c]pyridine-2-yl)methanamine hydrochloride (36 mg, 0.19 mmol) in DMF (1 mL) was added DIPEA (101 mg, 0.78 mmol) and HATU (74 mg, 0.19 mmol) and the reaction mixture was stirred at room temperature for 0.5 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 445 (5 mg, yield 6.7%) as a white solid. ¹H NMR (400 MHz, DMSO-d₆) δ 11.34 (s, 1H), 8.79 (t, J=5.7 Hz, 1H), 8.74 (s, 1H), 8.11 (d, J=5.7 Hz, 1H), 7.58 (d, J=7.8 Hz, 2H), 7.41-7.31 (m, 4H), 7.27 (t, J=7.4 Hz, 1H), 7.07 (d, J=3.6 Hz, 1H), 6.94 (s, 1H), 6.44 (s, 1H), 5.27 (d, J=7.0 Hz, 1H), 4.78-4.69 (m, 1H), 4.69-4.60 (m, 2H), 4.47 (d, J=5.5 Hz, 2H), 2.86-2.71 (m, 4H), 1.58 (d, J=6.6 Hz, 3H), 1.51-1.38 (m, 6H). LC/MS (ESI) (m/z): 568 (M+H)⁺. RT (Method A): 1.81 min.

Compounds 462 and 463 were prepared based on Scheme 131:

| # | Reactant A | Reactant B | Reactant C | Reactant D | Characterization Data |
|---|---|---|---|---|---|
| 462 | 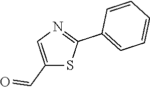 [SMILES: O=Cc1cnc(-c2ccccc2)s1] | 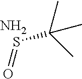 | 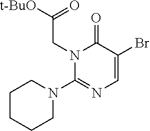 [SMILES: CC(C)(C)OC(=O)Cn1c(N2CCCCC2)ncc(Br)c1=O] | 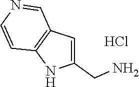 [SMILES: NCc1cc2cnccc2[nH]1 ; Cl] | ¹H NMR (400 MHz, CD₃OD) δ 8.72 (s, 1H), 8.11 (d, J = 5.9 Hz, 1H), 7.88- 7.81 (m, 2H), 7.77 (s, 1H), 7.48- 7.42 (m, 3H), 7.41 (d, J = 5.9 Hz, 1H), 7.01 (s, 1H), 6.59 (s, 1H), 4.90- 4.86 (m, 1H), 4.83-4.80 (m, 2H), 4.64-4.57 (m, 2H), 2.93-2.84 (m, 4H), 1.70 (d, J = 6.7 Hz, 3H), 1.58- 1.47 (m, 6H). LC/MS (ESI) m/z: 569 (M + H)⁺. RT (Method A): 1.50 min. |

| | | | | |
|---|---|---|---|---|
| 463^{a} | 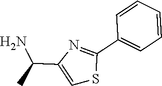 | 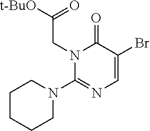 [SMILES: CC(C)(C)OC(=O)Cn1c(N2CCCCC2)ncc(Br)c1=O] | 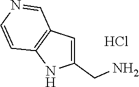 [SMILES: NCc1cc2cnccc2[nH]1 ; Cl] | ¹H NMR (400 MHz, CD₃OD) δ 8.69 (s, 1H), 8.08 (d, J = 5.8 Hz, 1H), 7.93 (s, 2H), 7.45 (s, 3H), 7.37 (d, J = 5.7 Hz, 1H), 7.34 (s, 1H), 6.92 (s, 1H), 6.56 (s, 1H), 4.82 (s, 2H), 4.71-4.65 (m, 1H), 4.61 (s, 2H), 2.91-2.83 (m, 4H), 1.65 (d, J = 6.6 Hz, 3H), 1.57- 1.47 (m, 6H). LC/MS (ESI) m/z: 569 (M + H)⁺. RT (Method A): 1.63 min. |

^{a}Steps 4-6 only.

### Scheme 132. Synthesis of (R)—N-((1H-Pyrrolo[3,2-c]pyridine-2-yl)methyl)-2-(2-(4-((7-aminoheptyl)oxy)phenyl)-5-((1-(dibenzo[b,d]furan-2-yl)ethyl)amino)-6-oxopyrimidin-1(6H)-yl)acetamide (Compound 451)

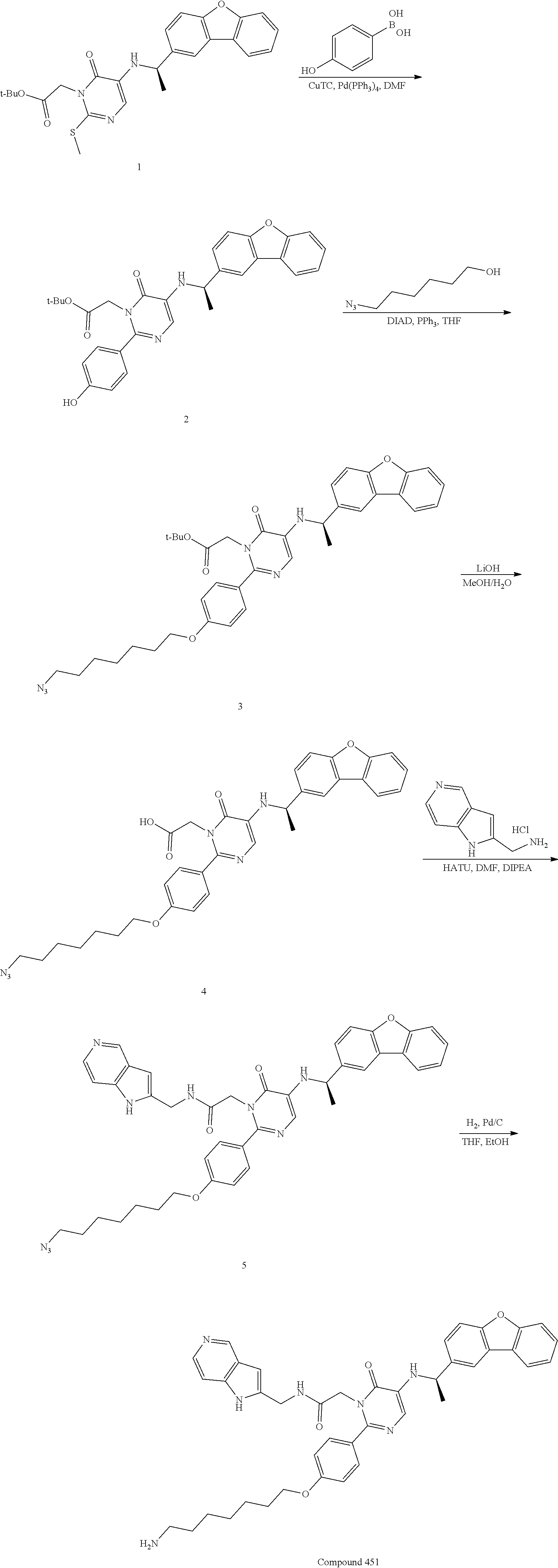

### Step 1: Tert-Butyl (R)-2-(5-((1-dibenzo[b,d]furan-2-yl)ethyl)amino)-2-(4-hydroxyphenyl)-6-oxopyrimidin-1(6H)-yl)acetate (2)

To a mixture of tert-butyl (R)-2-(5-((1-(dibenzo[b,d]furan-2-yl)ethyl)amino)-2-(methylthio)-6-oxopyrimidin-1(6H)-yl)acetate (1.0 g, 2.15 mmol) and (4-hydroxyphenyl)boronic acid (445 mg, 3.23 mmol) in DMF (20 mL) was added CuTC (903 mg, 4.73 mmol) and Pd(PPh₃)₄ (248 mg, 0.21 mmol) under N₂ atmosphere, the reaction mixture was degassed under N₂ atmosphere for three times and stirred at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 10-50% EtOAc in PE) to give the title compound (550 mg, yield 50%) as a yellow solid. LC/MS (ESI) m/z: 512 (M+H)⁺.

### Step 2: Tert-Butyl (R)-2-(2-(4-((7-azidoheptyl)oxy)phenyl)-5-((1-(dibenzo[b,d]furan-2-yl)ethyl)amino)-6-oxopyrimidin-1(6H)-yl)acetate (3)

To a solution of tert-butyl (R)-2-(5-((1-(dibenzo[b,d]furan-2-yl)ethyl)amino)-2-(4-hydroxyphenyl)-6-oxopyrimidin-1(6H)-yl)acetate (200 mg, 0.39 mmol) in THF (5 mL) was added PPh₃ (511 mg, 1.95 mmol) and DIAD (395 mg, 1.95 mmol) at 0° C. under N₂ atmosphere. The mixture was degassed under N₂ atmosphere for three times and stirred at 70° C. for 18 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 10-50% EtOAc in PE) to give the title compound (100 mg, yield 39%) as a yellow oil. LC/MS (ESI) m/z: 651 (M+H)⁺.

### Step 3: (R)-2-(2-(4-((7-Azidoheptyl)oxy)phenyl)-5-((1-(dibenzo[b,d]furan-2-yl)ethyl) amino)-6-oxopyrimidin-1(6H)-yl)acetic acid (4)

To a mixture of tert-butyl (R)-2-(2-(4-((7-azidoheptyl)oxy)phenyl)-5-((1-(dibenzo[b,d]furan-2-yl)ethyl)amino)-6-oxopyrimidin-1(6H)-yl)acetate (100 mg, 0.15 mmol) in MeOH/water (5.0 mL, v/v=2/1) was added LiOH (36 mg, 1.5 mmol) and the reaction mixture was stirred at 60° C. for 16 hours. The mixture was acidified with 1 N aq. HCl to pH˜3 and extracted with EtOAc twice. The combined organic layers were dried over anhydrous Na₂SO₄, filtered, and concentrated under reduced pressure to give the title compound (91 mg, yield 99%) as a yellow solid, which was used directly in the next step without further purification. LC/MS (ESI) m/z: 595 (M+H)⁺.

### Step 4: (R)—N-((1H-Pyrrolo[3,2-c]pyridine-2-yl)methyl)-2-(2-(4-((7-azidoheptyl)oxy) phenyl)-5-((1-(dibenzo[b,d]furan-2-yl)ethyl)amino)-6-oxopyrimidin-1(6H)-yl) acetamide (5)

To a mixture of (R)-2-(2-(4-((7-azidoheptyl)oxy)phenyl)-5-((1-(dibenzo[b,d]furan-2-yl)ethyl)amino)-6-oxopyrimidin-1(6H)-yl)acetic acid (90 mg, 0.15 mmol) and (1H-pyrrolo[3,2-c]pyridin-2-yl)methanamine hydrochloride (55 mg, 0.30 mmol) in DMF (2 mL) was added DIPEA (96 mg, 0.75 mmol) and HATU (85 mg, 0.23 mmol) and the reaction mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (65 mg, yield 59.9%) as a white solid. LC/MS (ESI) m/z: 724 (M+H)⁺.

### Step 5: (R)—N-((1H-Pyrrolo[3,2-c]pyridine-2-yl)methyl)-2-(2-(4-((7-aminoheptyl)oxy) phenyl)-5-((1-(dibenzo[b,d]furan-2-yl)ethyl)amino)-6-oxopyrimidin-1(6H)-yl) acetamide (Compound 451)

To a mixture of (R)—N-((1H-pyrrolo[3,2-c]pyridine-2-yl)methyl)-2-(2-(4-((7-azidoheptyl)oxy)phenyl)-5-((1-(dibenzo[b,d]furan-2-yl)ethyl)amino)-6-oxopyrimidin-1(6H)-yl)acetamide (60 mg, 0.08 mmol) in THF/EtOH (2.5 mL, v/v=4/1) was added Pd/C (10 mg, 10% w.t.) under N₂ atmosphere, the reaction mixture was degassed under N₂ atmosphere for three times and stirred under a H₂ balloon at room temperature for 3 hours. The mixture was filtered, and the filtrate was concentrated under reduced pressure to dryness. The residue was purified by prep-HPLC to give Compound 451 (10 mg, yield 17.9%) as a white solid. ¹H NMR (400 MHz, DMSO-d₆) δ 11.48 (s, 1H), 8.86 (s, 1H), 8.74 (s, 1H), 8.40 (s, 2H), 8.22 (s, 1H), 8.11 (t, J=6.2 Hz, 2H), 7.70-7.65 (m, 2H), 7.62 (d, J=8.6 Hz, 1H), 7.51 (t, J=7.8 Hz, 1H), 7.40 (t, J=7.5 Hz, 1H), 7.34 (d, J=5.6 Hz, 1H), 7.28 (d, J=8.6 Hz, 2H), 6.96 (s, 1H), 6.79 (d, J=8.7 Hz, 2H), 6.33 (s, 1H), 5.85 (d, J=6.9 Hz, 1H), 4.65 (t, J=6.9 Hz, 1H), 4.54 (d, J=16.3 Hz, 1H), 4.47-4.40 (m, 3H), 3.85 (t, J=6.3 Hz, 2H), 2.73 (t, J=7.4 Hz, 2H), 1.72-1.63 (m, 2H), 1.60 (d, J=6.7 Hz, 3H), 1.55-1.47 (m, 2H), 1.40-1.28 (m, 6H). LC/MS (ESI) m/z: 698 (M+H)⁺. RT (Method A): 1.40 min.

Compound 452 was prepared based on Steps 2-5 in Scheme 132:

| # | Reactant A | Reactant B | Reactant C | Characterization Data |
|---|---|---|---|---|
| 452 | 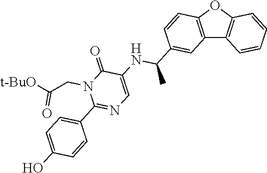 | 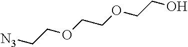 [SMILES: [N-]=[N+]=NCCOCCOCCO] | 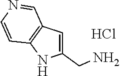 [SMILES: NCc1cc2cnccc2[nH]1 ; Cl] | ¹H NMR (400 MHz, DMSO-d₆) δ 8.82- 8.68 (m, 2H), 8.32 (s, 1H), 8.22 (s, 1H), 8.15-8.05 (m, 2H), 7.71-7.65 (m, 2H), 7.64-7.60 (m, 1H), 7.52 (t, J = 7.2 Hz, 1H), 7.40 (t, J = 7.6 Hz, 1H), 7.37-7.32 (m, 1H), 7.30 (d, J = 8.8 Hz, 2H), 6.96 (s, 1H), 6.83 (d, J = 8.8 Hz, 2H), 6.35- 6.31 (m, 1H), 5.86 (d, J = 7.1 Hz, 1H), 4.66 (t, J = 6.9 Hz, 1H), 4.54 (d, J = 16.0 Hz, 1H), 4.47-4.41 (m, 3H), 4.03- 3.99 (m, 2H), 3.73-3.70 (m, 2H), 3.58 (s, 2H), 3.56-3.55 (m, 2H), 3.51-3.49 (m, 2H), 2.84 (s, 2H), 1.60 (d, J = 6.7 Hz, 3H). LC/MS (ESI) m/z: 716 (M + H)⁺. RT (Method A): 1.24 min. |

### Scheme 133. Synthesis of (R)—N-((1H-Pyrrolo[3,2-c]pyridine-2-yl)methyl)-2-(6-oxo-5-((1-(2-phenyloxazol-5-yl)ethyl)amino)-2-(piperidin-1-yl)pyrimidin-1(6H)-yl)acetamide (Compound 453)

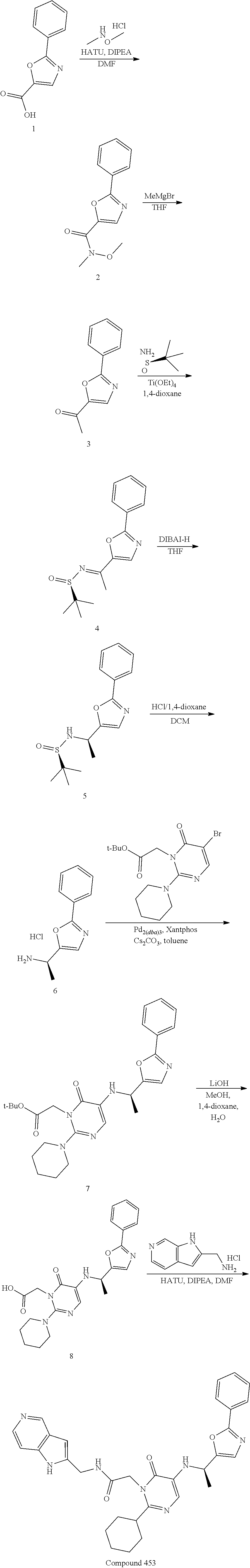

### Step 1: N-Methoxy-N-methyl-2-phenyloxazole-5-carboxamide (2)

To a mixture of 2-phenyloxazole-5-carboxylic acid (1 g, 5.29 mmol) and N,O-dimethylhydroxylamine hydrochloride (773 mg, 7.93 mmol) in DMF (10 mL) was added HATU (3.0 g, 7.93 mmol) and DIPEA (2.1 mg, 15.86 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-20% EtOAc in PE) to give the title compound (720 mg, yield 58.7%) as the white solid. LC/MS (ESI) m/z: 233 (M+H)⁺.

### Step 2: 1-(2-Phenyloxazol-5-yl)ethan-1-one (3)

To a mixture of N-methoxy-N-methyl-2-phenyloxazole-5-carboxamide (720 mg, 3.10 mmol) in DCM (10 mL) was added MeMgBr (5.2 mL, 15.5 mmol, 3M in toluene) dropwise at 0° C. under N₂ atmosphere and the reaction mixture was stirred at 0° C. for 2 hours. The mixture was acidified with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give title compound (550 mg, yield 94.8%) as a white solid. LC/MS (ESI) (m/z): 188 (M+H)⁺.

### Step 3: (R,E)-2-Methyl-N-(1-(2-phenyloxazol-5-yl)ethylidene)propane-2-sulfinamide (4)

To a mixture of 1-(2-phenyloxazol-5-yl)ethan-1-one (550 mg, 2.94 mmol) and I-2-methylpropane-2-sulfinamide (1.4 g, 11.75 mmol) in 1,4-dioxane (10 mL) was added Ti(OEt)₄ (2.7 g, 11.75 mmol) under N₂ atmosphere and the reaction mixture was stirred at 80° C. for 2 hours. The mixture was quenched with ice-water and extracted with EtOAc twice. The combined organic layers were washed with brine, dried over anhydrous Na₂SO₄, filtered, and concentrated to give title compound (820 mg, yield 96.1%) as a yellow solid. LC/MS (ESI) (m/z): 291 (M+H)⁺.

### Step 4: (R)-2-Methyl-N-(I-1-(2-phenyloxazol-5-yl)ethyl)propane-2-sulfinamide (5)

To a mixture of (R,E)-2-methyl-N-(1-(2-phenyloxazol-5-yl)ethylidene)propane-2-sulfinamide (820 mg, 2.82 mmol) in THF (10 mL) was added DIBAL-H (4.7 mL, 7.1 mmol, 1.5M in toluene) dropwise at −78° C. under N₂ atmosphere and the mixture was stirred at −78° C. for 2 hours. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give title compound (750 mg, yield 90.8%) as a white solid. LC/MS (ESI) (m/z): 293 (M+H)⁺.

### Step 5: (R)-1-(2-Phenyloxazol-5-yl)ethan-1-amine hydrochloride (6)

To a mixture of (R)-2-methyl-N—((R)-1-(2-phenyloxazol-5-yl)ethyl)propane-2-sulfinamide (750 mg, 2.56 mmol) in DCM (3 mL) was added HCl/1,4-dioxane (3 mL, 4M) and the reaction mixture was stirred at room temperature for 0.5 hour. The mixture was filtered, and the filter cake was washed with EtOAc three times, dried under vacuum to give title compound (520 mg, yield 90.2%) as a white solid. LC/MS (ESI) (m/z): 189 (M+H)⁺.

### Step 6: Tert-Butyl (R)-2-(6-oxo-5-((1-(2-phenyloxazol-5-yl)ethyl)amino)-2-(piperidin-1-yl)pyrimidin-1(6H)-yl)acetate (7)

To a mixture of tert-butyl 2-(5-bromo-6-oxo-2-(piperidin-1-yl)pyrimidin-1(6H)-yl)acetate (250 mg, 0.67 mmol) and (R)-1-(2-phenyloxazol-5-yl)ethan-1-amine hydrochloride (151 mg, 0.67 mmol) in toluene (5 mL) was added Cs₂CO₃ (656 mg, 2.01 mmol), Xantphos (78 mg, 0.13 mmol) and Pd₂(dba)₃ (61 mg, 0.067 mmol) under N₂ atmosphere, the reaction mixture was degassed under N₂ atmosphere for three times and stirred at 120° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-40% EtOAc in PE) to give title compound (75 mg, yield 23.3%) as a yellow solid. LC/MS (ESI) m/z: 480 (M+H)⁺.

### Step 7: (R)-2-(6-Oxo-5-((1-(2-phenyloxazol-5-yl)ethyl)amino)-2-(piperidin-1-yl) pyrimidin-1(6H)-yl)acetic acid (8)

To a solution of tert-butyl (R)-2-(6-oxo-5-((1-(2-phenyloxazol-5-yl)ethyl)amino)-2-(piperidin-1-yl)pyrimidin-1(6H)-yl)acetate (75 mg, 0.16 mmol) in MeOH/1,4-dioxane/water (3 mL, v/v/v=3/2/1) was added LiOH (15 mg, 0.63 mmol) and the reaction mixture was stirred at 80° C. overnight. The mixture was acidified with 1 N aq. HCl to pH˜4 and extracted with EtOAc twice. The combined organic layers were dried over anhydrous Na₂SO₄, filtered, and concentrated under reduced pressure to dryness to give the title compound (60 mg, yield 90.6%) as a yellow solid, which was used directly in the next step without further purification. LC/MS (ESI) m/z: 424 (M+H)⁺.

### Step 8: (R)—N-((1H-Pyrrolo[3,2-c]pyridine-2-yl)methyl)-2-(6-oxo-5-((1-(2-phenyloxazol-5-yl)ethyl)amino)-2-(piperidin-1-yl)pyrimidin-1(6H)-yl)acetamide (Compound 453)

To a mixture of (R)-2-(6-oxo-5-((1-(2-phenyloxazol-5-yl)ethyl)amino)-2-(piperidin-1-yl)pyrimidin-1(6H)-yl)acetic acid (60 mg, 0.14 mmol) and (1H-pyrrolo[3,2-c]pyridine-2-yl)methanamine hydrochloride (39 mg, 0.21 mmol) in DMF (3 mL) was added DIPEA (110 mg, 0.85 mmol) and HATU (59 mg, 0.16 mmol) and the reaction mixture was stirred at room temperature for 1 hour. The mixture was concentrated under reduced pressure to dryness. The residue was purified by prep-TLC (DCM:MeOH=6:1) and further purified by prep-HPLC to give the title compound (5.0 mg, yield 6.4%) as a white solid. ¹H NMR (400 MHz, CD₃OD) δ 8.69 (s, 1H), 8.09 (d, J=5.8 Hz, 1H), 7.98-7.92 (m, 2H), 7.50-7.45 (m, 3H), 7.37 (d, J=5.8 Hz, 1H), 7.11 (s, 2H), 6.56 (s, 1H), 4.81 (s, 2H), 4.79-4.75 (m, 1H), 4.59 (s, 2H), 2.94-2.84 (m, 4H), 1.68 (d, J=6.8 Hz, 3H), 1.58-1.47 (m, 6H). LC/MS (ESI) m/z: 553 (M+H)⁺. RT (Method A): 1.37 min.

### Scheme 134. Synthesis of (R)—N-((1H-Pyrrolo[3,2-c]pyridine-2-yl)methyl)-2-(2-morpholino-6-oxo-5-((1-(2-phenyloxazol-4-yl)ethyl)amino)pyrimidin-1(6H)-yl)acetamide (Compound 461)

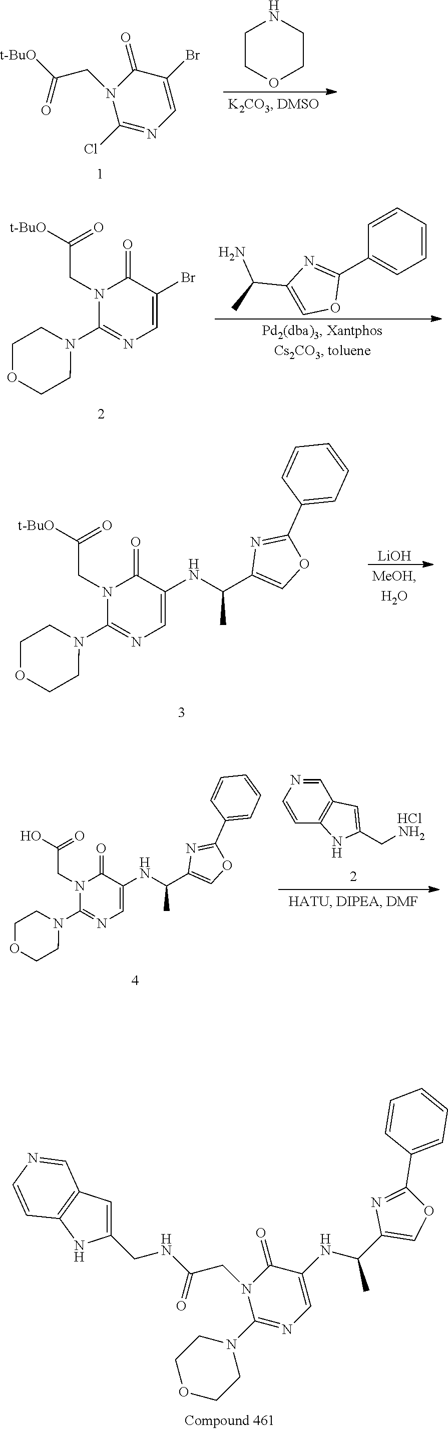

### Step 1: Tert-Butyl 2-(5-bromo-2-morpholino-6-oxopyrimidin-1(6H)-yl)acetate (2)

To a mixture of tert-butyl 2-(5-bromo-2-chloro-6-oxopyrimidin-1(6H)-yl)acetate (400 mg, 1.24 mmol) and morpholine (161 mg, 1.85 mmol) in DMSO (4 mL) was added K₂CO₃ (345 mg, 2.5 mmol) under N₂ atmosphere, the reaction mixture was stirred at 60° C. for 10 mins. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give the title compound (265 mg, yield 57.1%) as a yellow oil. LC/MS (ESI) m/z: 374 (M+H)⁺.

### Step 2: Tert-Butyl (R)-2-(2-morpholino-6-oxo-5-((1-(2-phenyloxazol-4-yl)ethyl)amino)pyrimidin-1(6H)-yl)acetate (3)

To a mixture of tert-butyl 2-(5-bromo-2-morpholino-6-oxopyrimidin-1(6H)-yl)acetate (220 mg, 0.59 mmol) and (R)-1-(2-phenyloxazol-4-yl)ethan-1-amine (133 mg, 0.71 mmol) in toluene (3 mL) was added Cs₂CO₃ (391 mg, 1.2 mmol), Xantphos (69 mg, 0.12 mmol) and Pd₂(dba)₃ (55 mg, 0.06 mmol) under N₂ atmosphere, the reaction mixture was degassed under N₂ atmosphere for three times and stirred at 110° C. for 10 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-40% EtOAc in PE) to give the title compound (65 mg, yield 22.9%) as a yellow solid. LC/MS (ESI) m/z: 482 (M+H)⁺.

### Step 3: (R)-2-(2-Morpholino-6-oxo-5-((1-(2-phenyloxazol-4-yl)ethyl)amino) pyrimidin-1(6H)-yl)acetic acid (4)

To a solution of tert-butyl (R)-2-(2-morpholino-6-oxo-5-((1-(2-phenyloxazol-4-yl)ethyl)amino)pyrimidin-1(6H)-yl)acetate (60 mg, 0.12 mmol) in MeOH/water (1 mL, v/v=1/1) was added LiOH (16 mg, 0.38 mmol) and the mixture was stirred at 50° C. overnight. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (46 mg, yield 90.1%) as a yellow solid, which was used directly in the next step without further purification. LC/MS (ESI) m/z: 426 (M+H)⁺.

### Step 4: (R)—N-((1H-Pyrrolo[3,2-c]pyridine-2-yl)methyl)-2-(2-morpholino-6-oxo-5-((1-(2-phenyloxazol-4-yl)ethyl)amino)pyrimidin-1(6H)-yl)acetamide (Compound 461)

To a mixture of (R)-2-(2-morpholino-6-oxo-5-((1-(2-phenyloxazol-4-yl)ethyl)amino) pyrimidin-1(6H)-yl)acetic acid (40 mg, 0.09 mmol) and (1H-pyrrolo[3,2-c]pyridin-2-yl)methanamine hydrochloride (28 mg, 0.15 mmol) in DMF (0.5 mL) was added DIPEA (38 mg, 0.3 mmol) and HATU (38 mg, 0.1 mmol), the reaction mixture was stirred at room temperature for 1 hour. The mixture was concentrated under reduced pressure to dryness. The residue was purified by prep-HPLC to give Compound 461 (13.0 mg, yield 24.9%) as a white solid. ¹H NMR (400 MHz, DMSO-d₆) δ 11.31 (s, 1H), 8.82 (t, J=5.6 Hz, 1H), 8.74 (s, 1H), 8.11 (d, J=5.7 Hz, 1H), 8.08 (s, 1H), 7.97 (m, 2H), 7.56-7.49 (m, 3H), 7.33 (d, J=5.6 Hz, 1H), 7.07 (s, 1H), 6.45 (s, 1H), 5.06 (d, J=8.0 Hz, 1H), 4.72 (s, 2H), 4.58-4.51 (m, 1H), 4.47 (d, J=5.5 Hz, 2H), 3.60-3.51 (m, 4H), 2.88-2.80 (m, 4H), 1.53 (d, J=6.7 Hz, 3H). LC/MS (ESI) m/z: 555 (M+H)⁺.

### Scheme 135. Synthesis of (R)—N-((1H-Pyrrolo[3,2-c]pyridine-2-yl)methyl)-2-(3-((1-(dibenzo[b,d]furan-2-yl)ethyl)amino)-2-oxo-6-(tetrahydro-2H-pyran-4-yl)pyrazin-1(2H)-yl)acetamide (Compound 471)

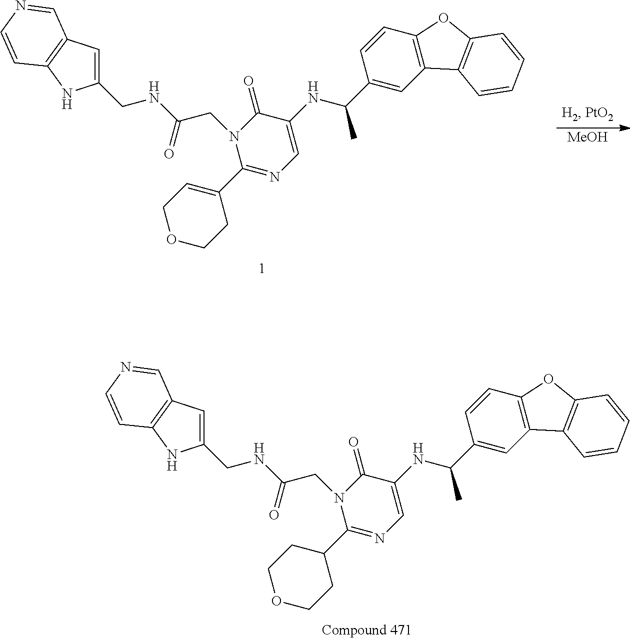 [SMILES: "MeOH" ; C[C@@H](Nc1cnc(C2=CCOCC2)n(CC(=O)NCc2cc3cnccc3[nH]2)c1=O)c1ccc2oc3ccccc3c2c1 ; C[C@@H](Nc1cnc(C2CCOCC2)n(CC(=O)NCc2cc3cnccc3[nH]2)c1=O)c1ccc2oc3ccccc3c2c1]

To a solution of (R)—N-((1H-pyrrolo[3,2-c]pyridine-2-yl)methyl)-2-(3-((1-(dibenzo[b,d]furan-2-yl)ethyl)amino)-6-(3,6-dihydro-2H-pyran-4-yl)-2-oxopyrazin-1(2H)-yl)acetamide (88 mg, 0.15 mmol) in MeOH (30 mL) was added PtO₂ (30 mg) and the mixture was degassed under N₂ atmosphere for three times and stirred under a H₂ balloon at 45° C. for 4 days. The mixture was filtered. The filtrate was concentrated under reduced pressure to dryness. The residue was purified by prep-HPLC to give Compound 471 (25.3 mg, yield 28.7%) as a white solid. ¹H NMR (400 MHz, DMSO-d₆) δ 11.32 (s, 1H), 8.81 (s, 1H), 8.73 (s, 1H), 8.16-8.09 (m, 3H), 7.67 (d, J=8.2 Hz, 1H), 7.63-7.56 (m, 2H), 7.50 (t, J=7.6 Hz, 1H), 7.39 (t, J=7.4 Hz, 1H), 7.35-7.30 (m, 2H), 6.64 (s, 1H), 6.43 (s, 1H), 5.27-5.19 (m, 1H), 4.79-4.68 (m, 2H), 4.46 (d, J=5.3 Hz, 2H), 3.79-3.72 (m, 2H), 3.29-3.18 (m, 3H), 1.67-1.59 (m, 2H), 1.58 (d, J=6.8 Hz, 3H), 1.53-1.43 (m, 2H). LC/MS (ESI) m/z: 659 (M+H)⁺. RT (Method A): 1.76 min.

### Scheme 136. Synthesis of (R)—N-((1H-Pyrrolo[3,2-c]pyridine-2-yl)methyl)-2-(5-((1-(2-(2-fluorophenyl)oxazol-4-yl)ethyl)amino)-6-oxo-2-(piperidin-1-yl)pyrimidin-1(6H)-yl) acetamide (Compound 475)

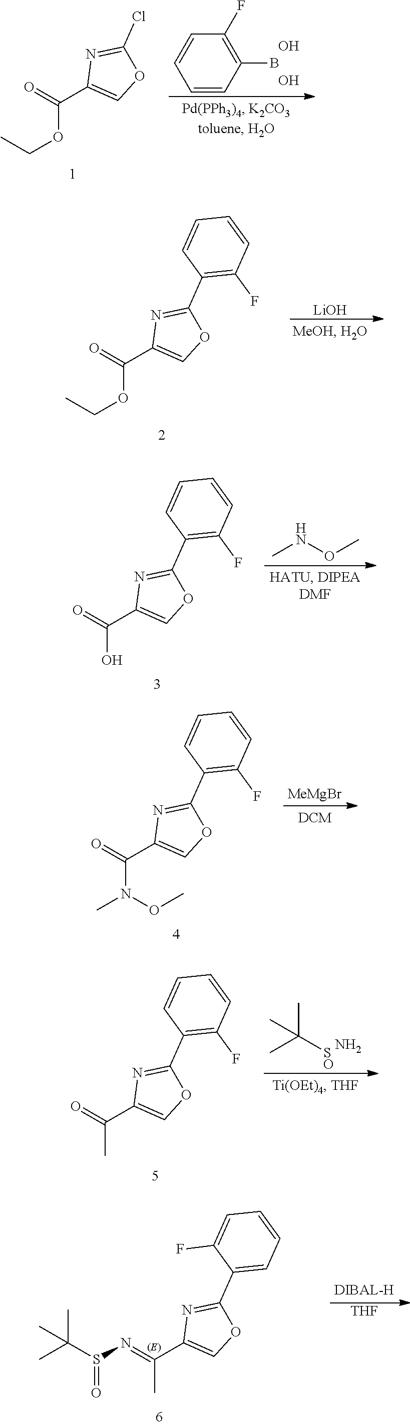

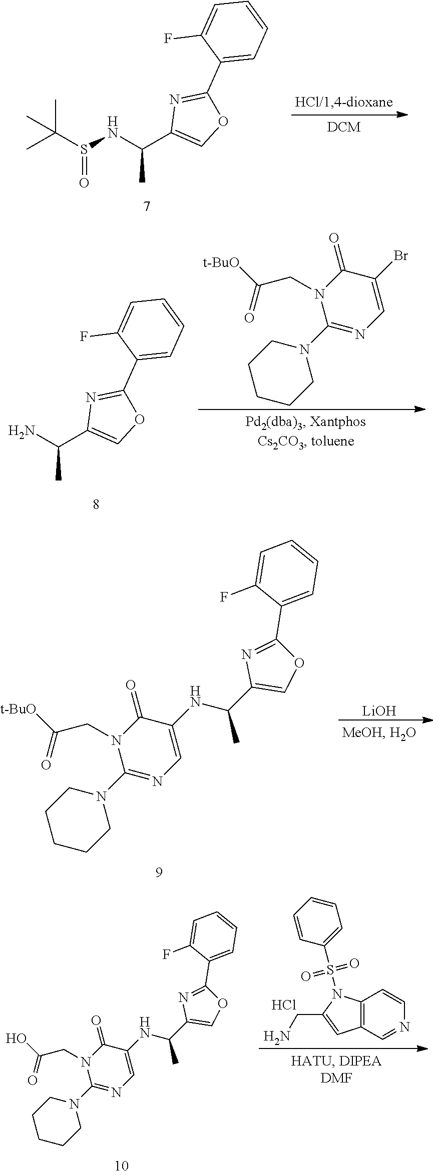

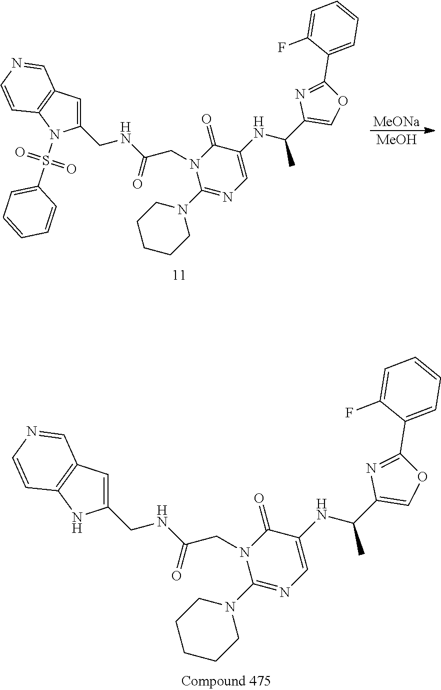 [SMILES: "MeONa" ; "MeOH" ; C[C@@H](Nc1cnc(N2CCCCC2)n(CC(=O)NCc2cc3cnccc3n2S(=O)(=O)c2ccccc2)c1=O)c1coc(-c2ccccc2F)n1 ; C[C@@H](Nc1cnc(N2CCCCC2)n(CC(=O)NCc2cc3cnccc3[nH]2)c1=O)c1coc(-c2ccccc2F)n1]

### Step 1: Ethyl 2-(2-fluorophenyl)oxazole-4-carboxylate (2)

To a mixture of ethyl 2-chlorooxazole-4-carboxylate (20.00 g, 11.39 mmol) and (2-fluorophenyl)boronic acid (31.87 g, 22.77 mmol) in toluene (200 mL) and water (30 mL) was added K₂CO₃ (31.44 g, 22.77 mmol) and Pd(PPh₃)₄ (6.58 g, 5.69 mmol) under N₂ atmosphere and the reaction mixture was stirred at 90° C. for 4 hours. The mixture was diluted with EtOAc, washed with water and brine, dried over anhydrous Na₂SO₄, filtered, and concentrated under reduced pressure to dryness. The residue was purified by flash chromatography (silica gel, 0-15% EtOAc in PE) to give the title compound (17.0 g, yield 61.2%) as a yellow oil. LC/MS (ESI) m/z: 236 (M+H)⁺.

### Step 2: 2-(2-Fluorophenyl)oxazole-4-carboxylic acid (3)

To a solution of ethyl 2-(2-fluorophenyl)oxazole-4-carboxylate (8.00 g, 34.04 mmol) in MeOH (60 mL) and water (20 mL) was added LiOH (2.45 g, 102.12 mmol). The reaction mixture was stirred at room temperature overnight. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (6.6 g, yield 93.7%) as a white solid, which was used directly in the next reaction without further purification. LC/MS (ESI) m/z: 208 (M+H)⁺.

### Step 3: 2-(2-Fluorophenyl)-N-methoxy-N-methyloxazole-4-carboxamide (4)

To a mixture of 2-(2-fluorophenyl)oxazole-4-carboxylic acid (6.0 g, 28.98 mmol) and N,O-dimethylhydroxylamine hydrochloride (5.7 g, 57.96 mmol) in DMF (60 mL) was added DIPEA (22.4 g, 173.88 mmol) and HATU (13.2 g, 35.97 mmol) and the mixture was stirred at room temperature for 4 hours. The mixture was diluted with EtOAc, washed with water and brine, dried over anhydrous Na₂SO₄, filtered, and concentrated to dryness under reduced pressure. The residue was purified by flash chromatography (silica gel, 0-22% EtOAc in PE) to give the title compound (6.0 g, yield 82.8%) as a yellow solid. LC/MS (ESI) m/z: 251 (M+H)⁺.

### Step 4: 1-(2-(2-Fluorophenyl)oxazol-4-yl)ethan-1-one (5)

To a solution of 2-(2-fluorophenyl)-N-methoxy-N-methyloxazole-4-carboxamide (6.0 g, 24.00 mmol) in DCM (70 mL) was added MeMgBr (40 mL, 120 mmol, 3.0 M) dropwise at 0° C. under N₂ atmosphere and the mixture was stirred under N₂ atmosphere at 0° C. for 1 hour. The mixture was quenched with saturated aq. NH₄Cl solution at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-9% EtOAc in PE) to give the title compound (2.0 g, yield 40.7%) as a yellow solid. LC/MS (ESI) m/z: 206 (M+H)⁺.

### Step 5: (R,E)-N-(1-(2-(2-Fluorophenyl)oxazol-4-yl)ethylidene)-2-methylpropane-2-sulfinamide (6)

To a mixture of 1-(2-(2-fluorophenyl)oxazol-4-yl)ethan-1-one (2.0 g, 9.76 mmol) and (R)-2-methylpropane-2-sulfinamide (3.55 g, 29.29 mmol) in THF (20 mL) was added Ti(OEt)₄ (6.67 g, 29.29 mmol) and the mixture was stirred at 70° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-21% EtOAc in PE) to give the title compound (2.47 g, yield 82.3%) as a yellow solid. LC/MS (ESI) (m/z): 309 (M+H)⁺.

### Step 6: (R)N—((R)-1-(2-(2-Fluorophenyl)oxazol-4-yl)ethyl)-2-methylpropane-2-sulfinamide (7)

To a solution of (R,E)-N-(1-(2-(2-fluorophenyl)oxazol-4-yl)ethylidene)-2-methylpropane-2-sulfinamide (2.47 g, 8.02 mmol) in THF (30 mL) was added DIBAL-H (16 mL, 24 mmol, 1.5 M in THF) dropwise under N₂ atmosphere at −78° C. and the mixture was stirred under N₂ atmosphere at −78° C. for 15 minutes. The mixture was quenched with saturated aq. Potassium sodium tartrate solution at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-35% EtOAc in PE) to give the title compound (2.2 g, yield 88.7%) as a yellow oil. LC/MS (ESI) m/z: 311 (M+H)⁺.

### Step 7: (R)-1-(2-(2-Fluorophenyl)oxazol-4-yl)ethan-1-amine hydrochloride (8)

To a solution of (R)—N—((R)-1-(2-(2-fluorophenyl)oxazol-4-yl)ethyl)-2-methylpropane-2-sulfinamide (2.2 g, 7.09 mmol) in DCM (10 mL) was added HCl/1,4-dioxane (20 mL, 4M) and the reaction mixture was stirred under N₂ atmosphere at room temperature for 3 hours. The reaction mixture was concentrated under reduced pressure to dryness to give the title compound (1.5 g, yield 87.7%) as a white solid, which was used directly in the next step without further purification. LC/MS (ESI) m/z: 207 (M+H)⁺.

### Step 8: Tert-Butyl (R)-2-(5-((1-(2-(2-fluorophenyl)oxazol-4-yl)ethyl)amino)-6-oxo-2-(piperidin-1-yl)pyrimidin-1(6H)-yl)acetate (9)

To a mixture of (R)-1-(2-(2-fluorophenyl)oxazol-4-yl)ethan-1-amine hydrochloride (300 mg, 1.45 mmol) and tert-butyl 2-(5-bromo-6-oxo-2-(piperidin-1-yl)pyrimidin-1(6H)-yl)acetate (540 mg, 1.45 mmol) in toluene (8 mL) was added Cs₂CO₃ (1.4 g, 4.35 mmol), Xantphos (84 mg, 0.14 mmol) and Pd₂(dba)₃ (133 mg, 0.14 mmol) under N₂ atmosphere and the reaction mixture was stirred at 120° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-45% EtOAc in PE) to give the title compound (80 mg, yield 11.1%) as a yellow solid. LC/MS (ESI) m/z: 498 (M+H)⁺.

### Step 9: l-2-(5-((1-(2-(2-Fluorophenyl)oxazol-4-yl)ethyl)amino)-6-oxo-2-(piperidin-1-yl)pyrimidin-1(6H)-yl)acetic acid (10)

To a solution of tert-butyl (R)-2-(5-((1-(2-(2-fluorophenyl)oxazol-4-yl)ethyl)amino)-6-oxo-2-(piperidin-1-yl)pyrimidin-1(6H)-yl)acetate (80 mg, 0.16 mmol) in MeOH (2 mL), THF (2 mL) and water (2 mL) was added LiOH (12 mg, 0.50 mmol). The reaction mixture was stirred at 60° C. overnight. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (60 mg, yield 70.9%) as a yellow solid, which was used directly in the next step without further purification. LC/MS (ESI) m/z: 442 (M+H)⁺.

### Step 10: (R)-2-(5-((1-(2-(2-Fluorophenyl)oxazol-4-yl)ethyl)amino)-6-oxo-2-(piperidin-1-yl)pyrimidin-1(6H)-yl)-N-((1-(phenylsulfonyl)-1H-pyrrolo[3,2-c]pyridine-2-yl)methyl) acetamide (11)

To a mixture of (R)-2-(5-((1-(2-(2-fluorophenyl)oxazol-4-yl)ethyl)amino)-6-oxo-2-(piperidin-1-yl)pyrimidin-1(6H)-yl)acetic acid (60 mg, 0.14 mmol) and (1-(phenylsulfonyl)-1H-pyrrolo[3,2-c]pyridine-2-yl)methanamine hydrochloride (58 mg, 0.20 mmol) in DMF (3 mL) was added DIPEA (105 mg, 0.81 mmol) and HATU (62 mg, 0.16 mmol) and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=15:1) to give the title compound (45 mg, yield 46.8%) as a yellow solid. LC/MS (ESI) (m/z): 711 (M+H)⁺.

### Step 11: (R)—N-((1H-Pyrrolo[3,2-c]pyridine-2-yl)methyl)-2-(5-((1-(2-(2-fluorophenyl) oxazol-4-yl)ethyl)amino)-6-oxo-2-(piperidin-1-yl)pyrimidin-1(6H)-yl)acetamide (Compound 475)

To a solution of (R)-2-(5-((1-(2-(2-fluorophenyl)oxazol-4-yl)ethyl)amino)-6-oxo-2-(piperidin-1-yl)pyrimidin-1(6H)-yl)-N-((1-(phenylsulfonyl)-1H-pyrrolo[3,2-c]pyridine-2-yl)methyl)acetamide (45 mg, 0.06 mmol) in MeOH (3 mL) was added MeONa (0.18 mL, 0.18 mmol, 1.0 M in MeOH) and the mixture was stirred at room temperature for 3 hours. The mixture filtered, and the filtrate was concentrated under reduced pressure to dryness. The residue was purified by prep-HPLC to give Compound 475 (1.1 mg, yield 3.05%) as a white solid. ¹H NMR (400 MHz, CD₃OD) δ 8.87 (s, 1H), 8.49 (s, 1H), 8.19 (d, J=6.2 Hz, 1H), 8.01-7.97 (m, 1H), 7.89 (s, 1H), 7.64 (d, J=6.2 Hz, 1H), 7.55-7.51 (m, 1H), 7.32-7.26 (m, 2H), 7.02 (s, 1H), 6.78 (s, 1H), 4.84 (s, 2H), 4.65 (s, 2H), 4.59-4.55 (m, 1H), 2.93-2.88 (m, 4H), 1.63 (d, 3H), 1.59-1.52 (m, 6H). LC/MS (ESI) (m/z): 571 (M+H)⁺.

### Scheme 137. Synthesis of (R)—N-((1H-Pyrrolo[3,2-c]pyridine-2-yl)methyl)-2-(6-oxo-5-((1-(4-phenyloxazol-2-yl)ethyl)amino)-2-(piperidin-1-yl)pyrimidin-1(6H)-yl) acetamide (Compound 487)

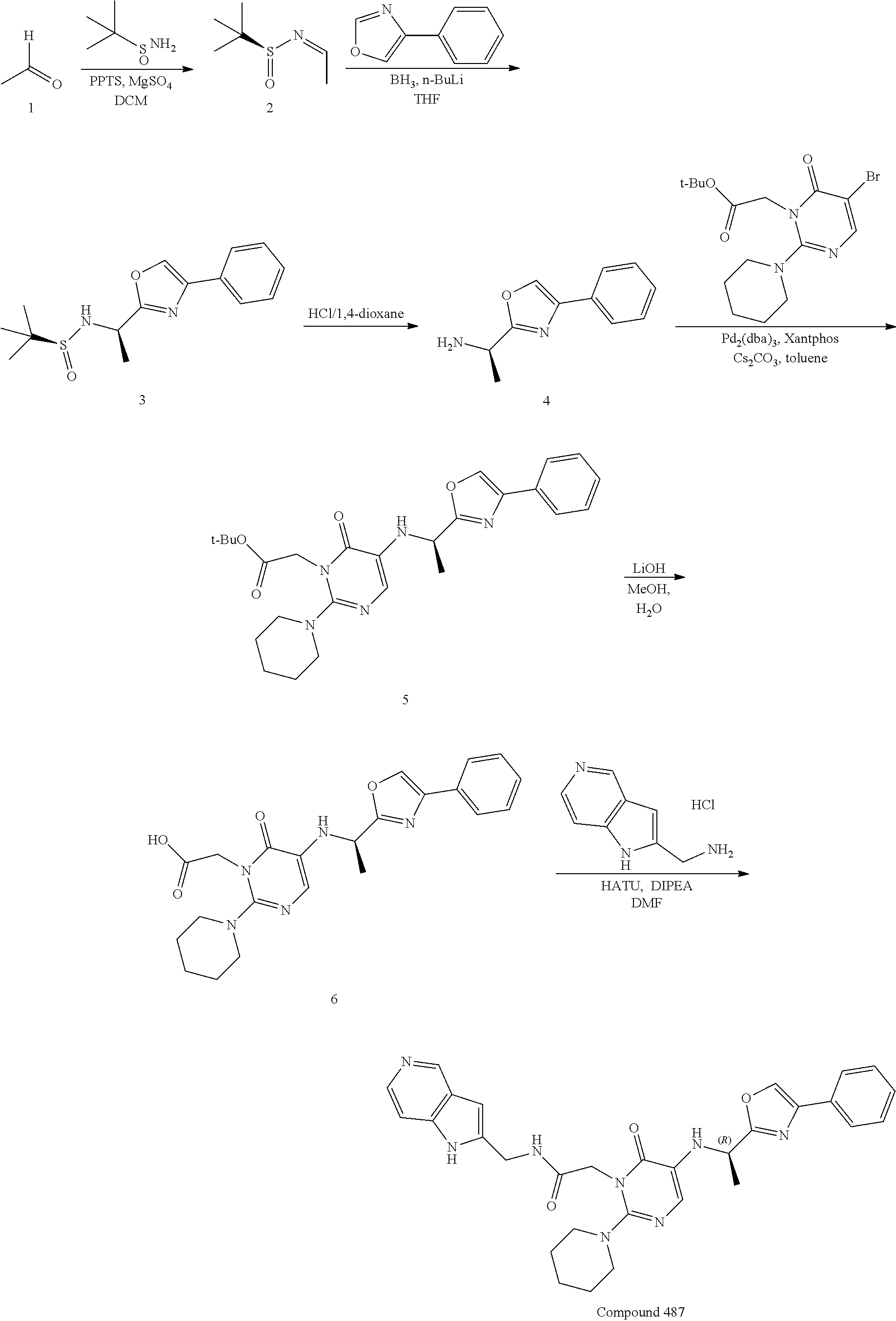

### Step 1: (S,Z)—N-Ethylidene-2-methylpropane-2-sulfinamide (2)

To a mixture of (S)-2-methylpropane-2-sulfinamide (5 g, 41.32 mmol) in DCM (50 mL) was added PPTS (515 mg, 41.32 mmol) and MgSO₄ (24.4 g, 205 mmol). The reaction mixture was stirred at room temperature for 0.5 hour. Then acetaldehyde (5.41 g, 123 mmol) added dropwise to the reaction mixture and the resulting mixture was stirred at room temperature overnight. The mixture was filtered, through a pad of celite, and the filter cake was washed with CH₂Cl₂. The filtrate was concentrated under reduced pressure to dryness. The residue was purified by flash chromatography (silica gel, 10-40% EtOAc in PE) to give the title compound (5.5 g, yield 91.66%) as a colorless oil. LC/MS (ESI) m/z: 148 (M+H)⁺.

### Step 2: (S)-2-Methyl-N—((R)-1-(4-phenyloxazol-2-yl)ethyl)propane-2-sulfinamide (3)

To a mixture of 4-phenyloxazole (650 mg, 4.42 mmol) in THF (6.5 mL) was added BH₃·THF (3.60 mL, 1.0 M) dropwise at −78° C. under N₂ atmosphere. The reaction mixture was stirred at room temperature for 1 hour. The n-BuLi (3.80 mL, 1.0 M) was added dropwise to the reaction mixture and the mixture was stirred at −78° C. for 2 hours. Afterwards, a solution of (S,Z)—N-ethylidene-2-methylpropane-2-sulfinamide (500 mg, 3.40 mmol) in THF (5 mL) was added dropwise to the above mixture and the resulting mixture was stirred at −78° C. for 2 hours. The mixture was quenched with saturated aq. NH₄Cl solution, and the layers were separated. The organic layer was washed with brine, dried over anhydrous Na₂SO₄, filtered, and concentrated under reduced pressure to dryness. The residue was purified by flash chromatography (silica gel, 10-35% EtOAc in PE) to give the title compound (800 mg, yield 79.6%) as a yellow solid. LC/MS (ESI) m/z: 293 (M+H)⁺.

### Step 3: (R)-1-(4-Phenyloxazol-2-yl)ethan-1-amine (4)

To a mixture of (S)-2-methyl-N—((R)-1-(4-phenyloxazol-2-yl)ethyl)propane-2-sulfinamide (750 mg, 2.60 mmol) in EtOAc (8 mL) and HCl/1,4-dioxane (3.0 mL, 4M) was stirred under N₂ atmosphere at room temperature for 2 hours. The reaction mixture was concentrated under reduced pressure to dryness to give the title compound (500 mg, yield 90.0%) as a white solid, which was used directly in the next reaction without further purification. LC/MS (ESI) m/z: 189 (M+H)⁺.

### Step 4: Tert-Butyl (R)-2-(6-oxo-5-((1-(4-phenyloxazol-2-yl)ethyl)amino)-2-(piperidin-1-yl)pyrimidin-1(6H)-yl)acetate (4)

To a mixture of tert-butyl 2-(5-bromo-6-oxo-2-(piperidin-1-yl)pyrimidin-1(6H)-yl)acetate (200 mg, 0.54 mmol) and (R)-1-(4-phenyloxazol-2-yl)ethan-1-amine (180 mg, 0.80 mmol) in toluene (3 mL) was added Cs₂CO₃ (528 mg, 1.62 mmol), Xantphos (62 mg, 0.11 mmol) and Pd₂(dba)₃ (37 mg, 0.05 mmol) under N₂ atmosphere, the reaction mixture was degassed under N₂ atmosphere for three times and stirred at 105° C. overnight. The mixture was diluted with EtOAc, washed with water and brine, dried over anhydrous Na₂SO₄, filtered, and concentrated under reduced pressure to dryness. The residue was purified by flash chromatography (silica gel, 30-85% EtOAc in PE) to give the title compound (100 mg, yield 38.7%) as a yellow solid. LC/MS (ESI) m/z: 480 (M+H)⁺.

### Step 5: (R)-2-(6-Oxo-5-((1-(4-phenyloxazol-2-yl)ethyl)amino)-2-(piperidin-1-yl) pyrimidin-1(6H)-yl)acetic acid (6)

To a mixture of tert-butyl (R)-2-(6-oxo-5-((1-(4-phenyloxazol-2-yl)ethyl)amino)-2-(piperidin-1-yl)pyrimidin-1(6H)-yl)acetate (90 mg, 0.19 mmol) in MeOH (0.5 mL) and water (0.5 mL) was added LiOH·H₂O (40 mg, 0.95 mmol) and the reaction mixture was stirred at 50° C. for 12 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (55 mg, yield 65.6%) as a yellow oil, which was used directly in the next step without further purification. LC/MS (ESI) m/z: 424 (M+H)⁺.

### Step 6: (R)—N-((1H-Pyrrolo[3,2-c]pyridine-2-yl)methyl)-2-(6-oxo-5-((1-(4-phenyloxazol-2-yl)ethyl)amino)-2-(piperidin-1-yl)pyrimidin-1(6H)-yl)acetamide (Compound 487)

To a mixture of (R)-2-(6-oxo-5-((1-(4-phenyloxazol-2-yl)ethyl)amino)-2-(piperidin-1-yl)pyrimidin-1(6H)-yl)acetic acid (50 mg, 0.12 mmol) and (1H-pyrrolo[3,2-c]pyridine-2-yl)methanamine hydrochloride (33 mg, 0.18 mmol) in DMF (0.5 mL) was added DIPEA (47 mg, 0.36 mmol) and HATU (46 mg, 0.12 mmol) and the reaction mixture was stirred at room temperature for 0.5 hour. The mixture was concentrated under reduced pressure to dryness. The residue was purified by prep-HPLC (C₁₈, 40-95% Macon in water with 0.1% NH₃·H₂O) to give Compound 487 (1.5 mg, yield 2.2%) as a white solid. ¹H NMR (400 MHz, DMSO-d₆) δ 11.33 (s, 1H), 8.84-8.78 (m, 1H), 8.72 (s, 1H), 8.54 (s, 1H), 8.10 (d, J=5.7 Hz, 1H), 7.76 (d, J=7.3 Hz, 2H), 7.47-7.39 (m, 2H), 7.35-7.30 (m, 2H), 7.04 (s, 1H), 6.43 (s, 1H), 5.38 (d, J=7.9 Hz, 1H), 4.79-4.72 (m, 1H), 4.67 (s, 2H), 4.47 (d, J=5.5 Hz, 2H), 2.88-2.75 (m, 4H), 1.60 (d, J=6.8 Hz, 3H), 1.52-1.39 (m, 6H). LC/MS (ESI) m/z: 553 (M+H)⁺. RT (Method A): 1.57 min.

Compound 487 was prepared based on Steps 2-6 in Scheme 137:

| # | Reactant A | Reactant B | Reactant C | Reactant D | Characterization Data |
|---|---|---|---|---|---|
| 488 | 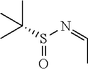 | 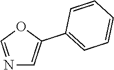 [SMILES: c1ccc(-c2cnco2)cc1] | 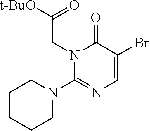 [SMILES: CC(C)(C)OC(=O)Cn1c(N2CCCCC2)ncc(Br)c1=O] | 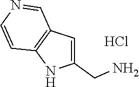 [SMILES: NCc1cc2cnccc2[nH]1 ; Cl] | ¹H NMR (400 MHz, CD₃OD) δ 8.70 (s, 1H), 8.10 (d, J = 5.8 Hz, 1H), 7.66 (d, J = 7.4 Hz, 2H), 7.44- 7.37 (m, 4H), 7.33 (t, J = 7.3 Hz, 1H), 7.11 (s, 1H), 6.56 (s, 1H), 4.81 (s, 2H), 4.78 (d, J = 6.8 Hz, 1H), 4.60 (s, 2H), 2.88 (d, J = 4.7 Hz, 4H), 1.72 (d, J = 6.9 Hz, 3H), 1.58-1.49 (m, 6H). LC/MS (ESI) m/z: 553 (M + H)⁺. RT (Method A): 1.53 min. |

### Scheme 138. Synthesis of N-((1H-Pyrrolo[3,2-c]pyridine-2-yl)methyl)-2-((3S*,6S*)-2-oxo-6-phenyl-3-((3-phenylpropyl)amino)piperidin-1-yl)acetamide (Compound 489)

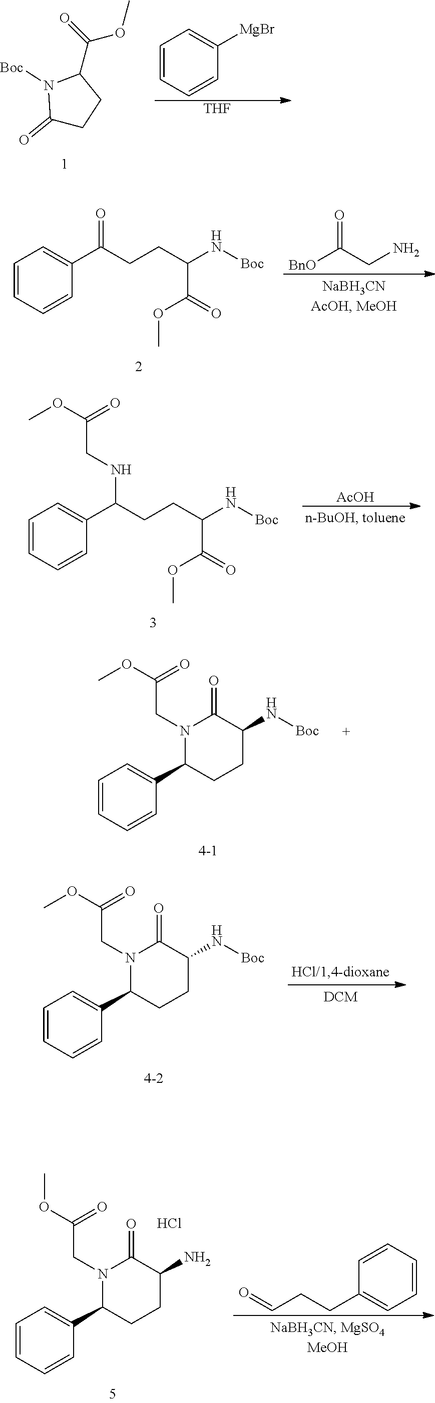

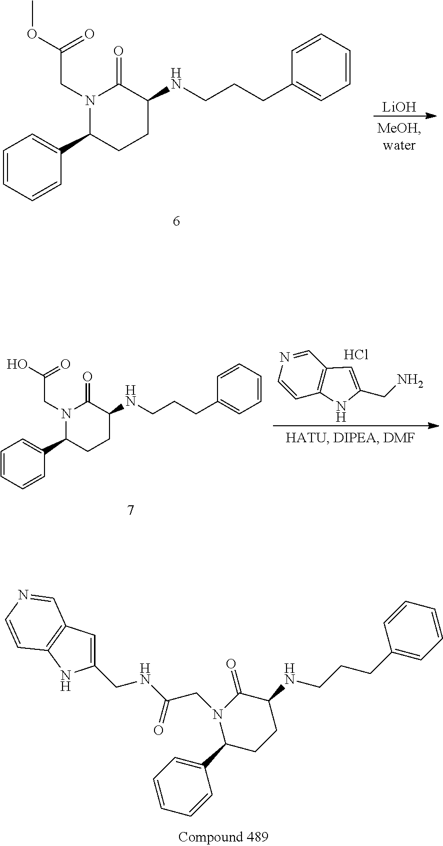

### Step 1: Methyl 2-((tert-butoxycarbonyl)amino)-5-oxo-5-phenylpentanoate (2)

To a mixture of 1-(tert-butyl) 2-methyl 5-oxopyrrolidine-1,2-dicarboxylate (10 g, 41.10 mmol) and TMEDA (7.17 g, 61.70 mmol) in THF (100 mL) was cooled to −78° C. and added phenylmagnesium bromide (22 mL, 2.8 M in THF, 61.66 mmol) dropwise via cannula at −78° C. The reaction mixture was stirred at −78° C. for 1 hour. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-18% EtOAc in PE) to give the title compound (12 g, yield 90.8%) as a white solid. LC/MS (ESI) m/z: 322 (M+H)⁺.

### Step 2: Methyl 2-((tert-butoxycarbonyl)amino)-5-((2-methoxy-2-oxoethyl)amino)-5-phenylpentanoate (3)

To a mixture of methyl 2-((tert-butoxycarbonyl)amino)-5-oxo-5-phenylpentanoate (10 g, 31.12 mmol), benzyl glycinate (7.71 g, 46.68 mmol) and AcOH (187 mg, 3.11 mmol) in MeOH (150 mL) was added NaBH₃CN (11.73 g, 186.66 mmol). The mixture was stirred at 50° C. for 12 hours. The mixture was filtered, and the filtrate was concentrated to dryness. The residue was diluted with EtOAc and washed with water. The combined organic layers were washed with brine, dried over anhydrous Na₂SO₄, filtered, and the filtrate concentrated to dryness. The residue was purified by flash chromatography (silica gel, 0-25% EtOAc in PE) to give the title compound (7 g, yield 57.0%) as a colorless oil. LC/MS (ESI) m/z: 395 (M+H)⁺.

### Step 3: Methyl 2-((3S*,6S*)-3-((tert-butoxycarbonyl)amino)-2-oxo-6-phenylpiperidin-1-yl)acetate and methyl 2-((3R*,6S*)-3-((tert-butoxycarbonyl) amino)-2-oxo-6-phenylpiperidin-1-yl)acetate (4)

To a solution of methyl 2-((tert-butoxycarbonyl)amino)-5-((2-methoxy-2-oxoethyl)amino)-5-phenylpentanoate (7 g, 17.75 mmol) in n-BuOH (50 mL) and toluene (50 mL) was added AcOH (107 mg, 1.78 mmol) and the mixture was stirred at 110° C. for 12 hours. The mixture was concentrated under reduced pressure to dryness. The residue was purified by flash chromatography (silica gel, 5-47% EtOAc in PE) to give methyl 2-((3S*,6S*)-3-((tert-butoxycarbonyl)amino)-2-oxo-6-phenylpiperidin-1-yl)acetate (1.5 g, yield 23.3%) and methyl 2-((3R*,6S*)-3-((tert-butoxycarbonyl)amino)-2-oxo-6-phenylpiperidin-1-yl)acetate (1.2 g, yield 18.7%) as a white solid. LC/MS (ESI) m/z: 363 (M+H)⁺.

### Step 4: Methyl 2-((3S*,6S*)-3-amino-2-oxo-6-phenylpiperidin-1-yl)acetate hydrochloride

To a mixture of Methyl 2-((3S*,6S*)-3-((tert-butoxycarbonyl)amino)-2-oxo-6-phenylpiperidin-1-yl)acetate (300 mg, 0.83 mmol) in DCM (2 mL) was added HCl/1,4-dioxane (2 mL, 4M) and the reaction mixture was stirred under N₂ atmosphere at room temperature for 1 hour. The reaction mixture was concentrated under reduced pressure to dryness to give the title compound (260 mg, yield 100%) as a white solid, which was used directly in the next reaction without further purification. LC/MS (ESI) m/z: 263 (M+H)⁺.

### Step 5: Methyl 2-((3S*,6S*)-2-oxo-6-phenyl-3-((3-phenylpropyl)amino)piperidin-1-yl)acetate (6)

To a mixture of methyl 2-((3S*,6S*)-3-amino-2-oxo-6-phenylpiperidin-1-yl)acetate (260 mg, 0.83 mmol), 3-phenylpropanal (112 mg, 0.76 mmol) and MgSO₄ (46 mg, 0.38 mmol) in MeOH (5 mL) was added NaBH₃CN (240 mg, 3.82 mmol) and the mixture was stirred at 50° C. for 1 hour. The mixture was filtered, and the filtrate was concentrated to dryness. The residue was purified by flash chromatography (silica gel, 0-5% MeOH in DCM) to give the title compound (130 mg, yield 41.1%) as a colorless oil. LC/MS (ESI) m/z: 381 (M+H)⁺.

### Step 6: 2-((3S*,6S*)-2-Oxo-6-phenyl-3-((3-phenylpropyl)amino)piperidin-1-yl)acetic acid (7)

To a solution of methyl 2-((3S*,6S*)-2-oxo-6-phenyl-3-((3-phenylpropyl)amino) piperidin-1-yl)acetate (130 mg, 0.34 mmol) in MeOH (2 mL) and water (2 mL) was added LiOH (57 mg, 1.36 mmol) and the reaction mixture was stirred at room temperature 1 hour. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (90 mg, yield 71.9%) as a yellow solid, which was used directly in the next step without further purification. LC/MS (ESI) m/z: 367 (M+H)⁺.

### Step 7: N-((1H-Pyrrolo[3,2-c]pyridine-2-yl)methyl)-2-((3S*,6S*)-2-oxo-6-phenyl-3-((3-phenylpropyl)amino)piperidin-1-yl)acetamide (Compound 489)

To a mixture of 2-((3S*,6S*)-2-oxo-6-phenyl-3-((3-phenylpropyl)amino)piperidin-1-yl)acetic acid (50 mg, 0.14 mmol) and (1H-pyrrolo[3,2-c]pyridin-2-yl)methanamine hydrochloride (30 mg, 0.16 mmol) in DMF (3 mL) was added DIPEA (88 mg, 0.68 mmol) and HATU (78 mg, 0.21 mmol) and the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC (C₁₈, 25-95% acetonitrile in H₂O with 0.1% NH₃·H₂O) to give Compound 489 (5.4 mg, yield 8.0%) as a white solid. ¹H NMR (400 MHz, DMSO-d₆) δ 11.30 (s, 1H), 8.71 (s, 1H), 8.38 (t, J=5.7 Hz, 1H), 8.10 (d, J=5.6 Hz, 1H), 7.40 (t, J=7.4 Hz, 2H), 7.33-7.28 (m, 2H), 7.28-7.23 (m, 2H), 7.21-7.13 (m, 5H), 6.36 (s, 1H), 4.77-4.73 (m, 1H), 4.57-4.51 (m, 1H), 4.48-4.37 (m, 2H), 3.26-3.21 (m, 1H), 3.06-2.99 (m, 1H), 2.66-2.59 (m, 2H), 2.58-2.53 (m, 2H), 2.40-2.22 (m, 2H), 1.86-1.69 (m, 4H), 1.49-1.38 (m, 1H). LC/MS (ESI) (m/z): 496 (M+H)⁺. RT (Method A): 0.89 min.

Compound 490 was prepared based on Steps 4-7 in Scheme 138:

| # | Reactant A | Reactant B | Reactant C | Characterization Data |
|---|---|---|---|---|
| 490 | 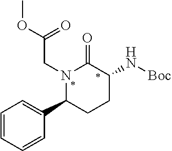 | 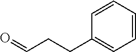 | 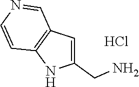 [SMILES: NCc1cc2cnccc2[nH]1 ; Cl] | ¹H NMR (400 MHz, CD₃OD) δ 8.68 (s, 1H), 8.08 (d, J = 5.8 Hz, 1H), 7.38-7.31 (m, 3H), 7.30-7.26 (m, 3H), 7.24 (d, J = 7.4 Hz, 2H), 7.20-7.12 (m, 3H), 6.46 (s, 1H), 4.72-4.66 (m, 1H), 4.56-4.45 (m, 2H), 4.25- 4.19 (m, 1H), 3.50-3.39 (m, 2H), 2.73-2.63 (m, 4H), 2.33-2.23 (m, 1H), 2.19-2.11 (m, 1H), 1.96-1.82 (m, 4H). LC/MS (ESI) m/z: 496 (M + H)⁺. RT (Method A)): 0.78 min. |

### Scheme 139. Synthesis of N-(4-(2-(((1H-Pyrrolo[3,2-c]pyridine-2-yl)methyl)amino)-2-oxoethyl)-5-morpholino-3-oxo-3,4-dihydropyrazin-2-yl)-2-phenyloxazole-4-carboxamide (Compound 492)

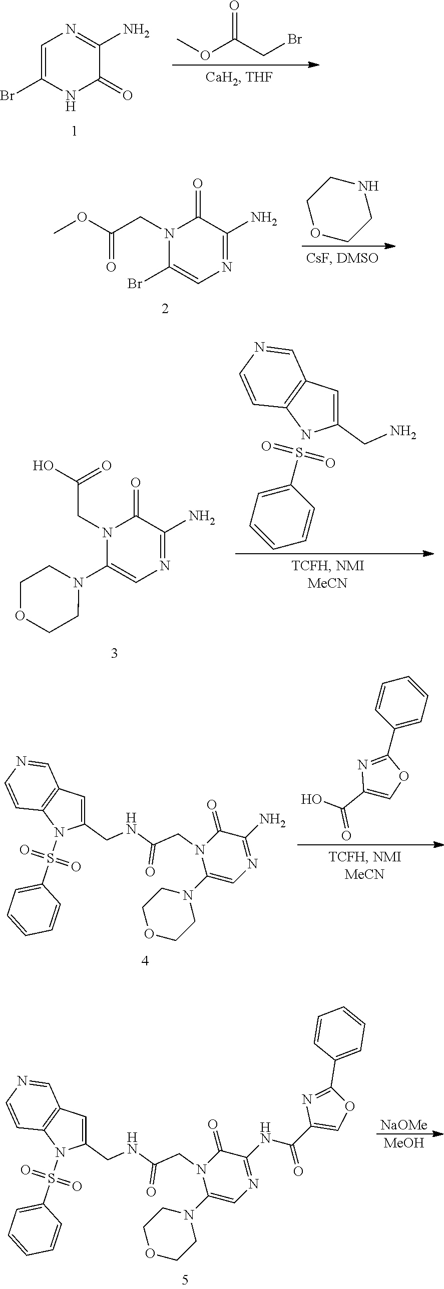

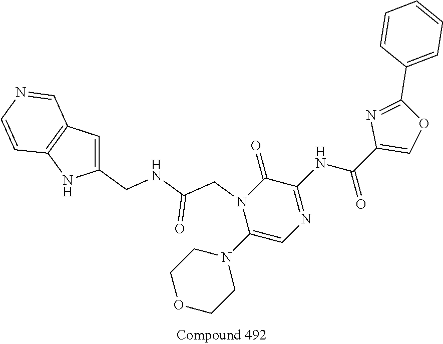 [SMILES: O=C(Cn1c(N2CCOCC2)cnc(NC(=O)c2coc(-c3ccccc3)n2)c1=O)NCc1cc2cnccc2[nH]1]

### Step 1: Methyl 2-(3-amino-6-bromo-2-oxopyrazin-1(2H)-yl)acetate (2)

To a solution of 3-amino-6-bromopyrazin-2(1H)-one (1.8 g, 9.47 mmol) in THF (20 mL) was added CaH₂ (598 mg, 14.21 mmol) and methyl 2-bromoacetate (2.17 g, 14.18 mmol) at 0° C. The mixture was stirred at 70° C. for 16 hours. The mixture was quenched with saturated aq. NH₄Cl solution at 0° C. and extracted EtOAc twice. The organic layer was washed with brine, dried over anhydrous Na₂SO₄, filtered, and concentrated under reduced pressure to dryness. The residue was purified by flash chromatography (silica gel, 10-42% EtOAc in PE) to give the title compound (460 mg, yield 18.5%) as a yellow solid. LC/MS (ESI) m/z: 262 (M+H)⁺.

### Step 2: 2-(3-Amino-6-morpholino-2-oxopyrazin-1(2H)-yl)acetic acid (3)

To a mixture of methyl 2-(3-amino-6-bromo-2-oxopyrazin-1(2H)-yl)acetate (400 mg, 1.53 mmol) and morpholine (267 mg, 3.06 mmol) in DMSO (4 mL) was added CsF (574 mg, 4.59 mmol) under N₂ atmosphere and the mixture was stirred at 100° C. overnight. The mixture was diluted with EtOAc, washed with brine, dried over anhydrous Na₂SO₄, filtered, and concentrated under reduced pressure to dryness. The residue was purified by prep-HPLC (C₁₈, 5%-98% MeCN in water with 0.1% TFA) to give the title compound (200 mg, yield 51.4%) as a yellow solid. LC/MS (ESI) m/z: 255 (M+H)⁺.

### Step 3: 2-(3-Amino-6-morpholino-2-oxopyrazin-1(2H)-yl)-N-((1-(phenylsulfonyl)-1H-pyrrolo[3,2-c]pyridine-2-yl)methyl)acetamide (4)

To a mixture of 2-(3-amino-6-morpholino-2-oxopyrazin-1(2H)-yl)acetic acid (190 mg, 0.74 mmol) and (1-(phenylsulfonyl)-1H-pyrrolo[3,2-c]pyridine-2-yl)methanamine (322 mg, 1.12 mmol) in MeCN (2 mL) was added TCFH (630 mg, 2.22 mmol) and NMI (364 mg, 4.44 mmol) and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-60% EtOAc in PE) to give the title compound (202 mg, yield 51.5%) as a yellow solid. LC/MS (ESI) m/z: 524 (M+H)⁺.

### Step 4: N-(5-Morpholino-3-oxo-4-(2-oxo-2-(((1-(phenylsulfonyl)-1H-pyrrolo[3,2-c]pyridin-2-yl)methyl)amino)ethyl)-3,4-dihydropyrazin-2-yl)-2-phenyloxazole-4-carboxamide (5)

To a mixture of 2-phenyloxazole-4-carboxylic acid (50 mg, 0.26 mmol) and 2-(3-amino-6-morpholino-2-oxopyrazin-1(2H)-yl)-N-((1-(phenylsulfonyl)-1H-pyrrolo[3,2-c]pyridine-2-yl)methyl)acetamide (200 mg, 0.38 mmol) in MeCN (2 mL) was added TCFH (223 mg, 0.79 mmol) and NMI (128 mg, 1.56 mmol) and the reaction mixture was stirred at room temperature for 2 hour. The mixture was concentrated under reduced pressure to dryness. The mixture was diluted with EtOAc, washed with water and brine, dried over anhydrous Na₂SO₄, filtered, and concentrated under reduced pressure to dryness. The residue was purified by flash chromatography (silica gel, 0-80% EtOAc in PE) to give the title compound (100 mg, yield 54.6%) as a yellow solid. LC/MS (ESI) m/z: 695 (M+H)⁺.

### Step 5: N-(4-(2-(((1H-Pyrrolo[3,2-c]pyridine-2-yl)methyl)amino)-2-oxoethyl)-5-morpholino-3-oxo-3,4-dihydropyrazin-2-yl)-2-phenyloxazole-4-carboxamide (Compound 492)

To a solution of N-(5-morpholino-3-oxo-4-(2-oxo-2-(((1-(phenylsulfonyl)-1H-pyrrolo[3,2-c]pyridine-2-yl)methyl)amino)ethyl)-3,4-dihydropyrazin-2-yl)-2-phenyloxazole-4-carboxamide (90 mg, 0.12 mmol) in MeOH (2 mL) was added MeONa (0.36 mL, 0.36 mmol, 1.0 M in MeOH) and the mixture was stirred at room temperature for 3 hours. The mixture filtered, and the filtrate was concentrated under reduced pressure to dryness. The residue was purified by prep-HPLC (C₁₈, 30-98% MeCN in water with 0.1% NH₄HCO₃) to give Compound 492 (2 mg, yield 2.7%) as a white solid. ¹H NMR (400 MHz, DMSO-d₆) δ 11.40 (s, 1H), 10.14 (s, 1H), 9.02-8.91 (m, 2H), 8.74 (s, 1H), 8.13-8.03 (m, 3H), 7.66-7.58 (m, 3H), 7.33 (d, J=5.3 Hz, 1H), 7.11 (s, 1H), 6.46 (s, 1H), 4.80 (s, 2H), 4.49 (s, 2H), 3.59 (m, 4H), 2.90 (m, 4H). LC/MS (ESI) (m/z): 555 (M+H)⁺.

### Scheme 140. Synthesis of (S)—N-((1H-Pyrrolo[3,2-c]pyridine-2-yl)methyl)-2-(3-((1-(dibenzo[b,d]furan-2-yl)-2-hydroxyethyl)amino)-2-oxo-6-(piperidin-1-yl)pyrazin-1(2H)-yl)acetamide (Compound 493)

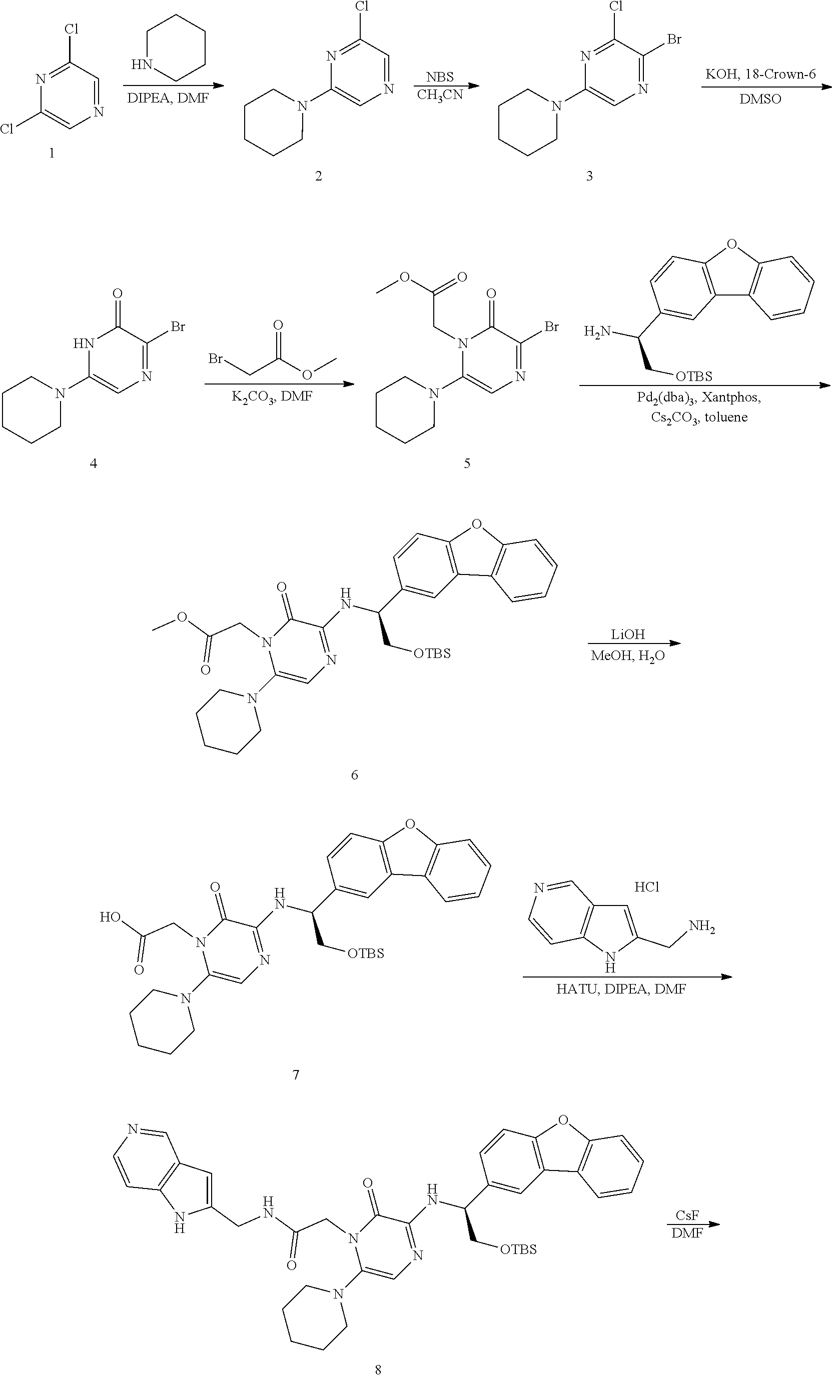

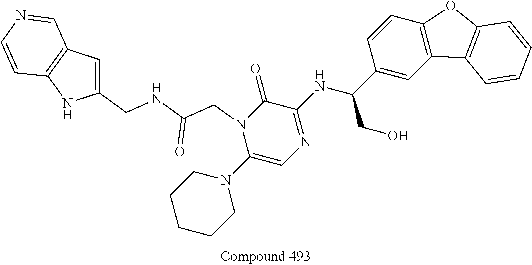 [SMILES: O=C(Cn1c(N2CCCCC2)cnc(N[C@H](CO)c2ccc3oc4ccccc4c3c2)c1=O)NCc1cc2cnccc2[nH]1]

### Step 1: 2-Chloro-6-(piperidin-1-yl)pyrazine (2)

To a mixture of 2,6-dichloropyrazine (5 g, 33.56 mmol) and piperidine (5.72 g, 67.17 mmol) in DMF (60 mL) was added DIPEA (8.68 g, 67.16 mmol) at room temperature under N₂ atmosphere and the reaction mixture was stirred at 80° C. for 6 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-7% EtOAc in PE) to give the title compound (4.8 g, yield 72.3%) as a colorless oil. LC/MS (ESI) m/z: 198 (M+H)⁺.

### Step 2: 2-Bromo-3-chloro-5-(piperidin-1-yl)pyrazine (3)

To a solution of 2-chloro-6-(piperidin-1-yl)pyrazine (4.8 g, 24.28 mmol) in CH₃CN (50 mL) was added NBS (5.19 mg, 29.16 mmol) at 0° C. and the reaction mixture was stirred at room temperature for 4 hours. The mixture was quenched with saturated aq. Na₂S₂O₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-5% EtOAc in PE) to give the title compound (5 g, yield 74.5%) as a yellow solid. LC/MS (ESI) m/z: 276 (M+H)⁺.

### Step 3: 3-Bromo-6-(piperidin-1-yl)pyrazin-2(1H)-one (4)

To a mixture of 2-bromo-3-chloro-5-(piperidin-1-yl)pyrazine (2.5 g, 9.04 mmol) and KOH (1.01 g, 18.00 mmol) in DMSO (30 mL) was added 18-Crown-6 (4.78 g, 18.08 mmol) and the mixture was stirred at 120° C. for 12 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-47% EtOAc in PE) to give the title compound (800 mg, yield 34.3%) as a yellow solid. LC/MS (ESI) m/z: 258 (M+H)⁺.

### Step 4: Methyl 2-(3-bromo-2-oxo-6-(piperidin-1-yl)pyrazin-1(2H)-yl)acetate (5)

To a solution of 3-bromo-6-(piperidin-1-yl)pyrazin-2(1H)-one (300 mg, 1.16 mmol) in DMF (5 mL) were added K₂CO₃ (321 mg, 2.32 mmol) and methyl 2-bromoacetate (356 mg, 2.33 mmol). The mixture was stirred at 70° C. for 16 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-42% EtOAc in PE) to give the title compound (120 mg, yield 31.3%) as a yellow solid. ¹H NMR (400 MHz, CDCl₃) δ 7.46 (s, 1H), 4.82 (s, 2H), 3.74 (s, 3H), 3.50-3.41 (m, 4H), 1.68-1.58 (m, 6H). LC/MS (ESI) (m/z): 330 (M+H)⁺.

### Step 5: Methyl (S)-2-(3-((2-((tert-butyldimethylsilyl)oxy)-1-(dibenzo[b,d]furan-2-yl)ethyl)amino)-2-oxo-6-(piperidin-1-yl)pyrazin-1(2H)-yl)acetate (6)

To a mixture of (S)-2-((tert-butyldimethylsilyl)oxy)-1-(dibenzo[b,d]furan-2-yl)ethan-1-amine (47 mg, 0.14 mmol), methyl 2-(3-bromo-2-oxo-6-(piperidin-1-yl)pyrazin-1(2H)-yl)acetate (55 mg, 0.17 mmol) and Cs₂CO₃ (135 mg, 0.41 mmol) in toluene (3 mL) were added Pd₂(dba)₃ (13 mg, 0.01 mmol) and Xantphos (16 mg, 0.03 mmol) under N₂ atmosphere. The mixture was degassed under N₂ atmosphere for three times and stirred under N₂ atmosphere at 100° C. for 12 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-32% EtOAc in PE) to give the title compound (62 mg, yield 76.3%) as a yellow solid. LC/MS (ESI) m/z: 591 (M+H)⁺.

### Step 6: (S)-2-(3-((2-((tert-Butyldimethylsilyl)oxy)-1-(dibenzo[b,d]furan-2-yl)ethyl) amino)-2-oxo-6-(piperidin-1-yl)pyrazin-1(2H)-yl)acetic acid (7)

To a solution of methyl (S)-2-(3-((2-((tert-butyldimethylsilyl)oxy)-1-(dibenzo[b,d]furan-2-yl)ethyl)amino)-2-oxo-6-(piperidin-1-yl)pyrazin-1(2H)-yl)acetate (62 mg, 0.10 mmol) in MeOH (3 mL) and water (1 mL) was added LiOH (18 mg, 0.43 mmol) and the mixture was stirred at room temperature for 1 hour. The mixture was acidified with 1 N aq. HCl to pH˜3 and extracted with EtOAc twice. The combined organic layers were washed with brine, dried over anhydrous Na₂SO₄, filtered, and concentrated under reduced pressure to dryness to give the title compound (58 mg, yield 95.8%) as a yellow solid, which was used directly in the next reaction without further purification. LC/MS (ESI) m/z: 577 (M+H)⁺.

### Step 7: (S)—N-((1H-Pyrrolo[3,2-c]pyridine-2-yl)methyl)-2-(3-((2-((tert-butyldimethylsilyl)oxy)-1-(dibenzo[b,d]furan-2-yl)ethyl)amino)-2-oxo-6-(piperidin-1-yl)pyrazin-1(2H)-yl)acetamide (8)

To a mixture of (S)-2-(3-((2-((tert-butyldimethylsilyl)oxy)-1-(dibenzo[b,d]furan-2-yl)ethyl)amino)-2-oxo-6-(piperidin-1-yl)pyrazin-1(2H)-yl)acetic acid (58 mg, 0.10 mmol) and (1H-pyrrolo[3,2-c]pyridine-2-yl)methanamine hydrochloride (22 mg, 0.12 mmol) in DMF (1 mL) was added DIPEA (65 mg, 0.50 mmol) and HATU (57 mg, 0.15 mmol) and the reaction mixture was stirred at room temperature for 0.5 hour. The mixture was concentrated under reduced pressure to dryness. The residue was purified by prep-TLC (DCM:MeOH=10:1) to give the title compound (30 mg, yield 42.3%) as a yellow solid. LC/MS (ESI) m/z: 706 (M+H)⁺.

### Step 8: (S)—N-((1H-Pyrrolo[3,2-c]pyridine-2-yl)methyl)-2-(3-((1-(dibenzo[b,d]furan-2-yl)-2-hydroxyethyl)amino)-2-oxo-6-(piperidin-1-yl)pyrazin-1(2H)-yl)acetamide (Compound 493)

To a solution of (S)—N-((1H-pyrrolo[3,2-c]pyridine-2-yl)methyl)-2-(3-((2-((tert-butyldimethylsilyl)oxy)-1-(dibenzo[b,d]furan-2-yl)ethyl)amino)-2-oxo-6-(piperidin-1-yl)pyrazin-1(2H)-yl)acetamide (30 mg, 0.04 mmol) in DMF (1 mL) was added CsF (19 mg, 0.13 mmol) and the mixture was stirred at 40° C. for 3 hours. The mixture was filtered, and the filtrate was concentrated to dryness. The residue was purified by prep-HPLC (C₁₈, 40%-98% MeCN in water with 0.1% NH₃·H₂O) to give Compound 493 (4.5 mg, yield 17.9%) as a yellow solid. ¹H NMR (400 MHz, CD₃OD) δ 8.63 (d, J=0.9 Hz, 1H), 8.07 (t, J=3.9 Hz, 2H), 7.98 (d, J=7.2 Hz, 1H), 7.56-7.52 (m, 2H), 7.50-7.35 (m, 3H), 7.33-7.29 (m, 2H), 7.03 (s, 1H), 6.42 (s, 1H), 5.24-5.18 (m, 1H), 4.94 (s, 1H), 4.93 (s, 1H), 4.62 (d, J=3.5 Hz, 2H), 3.97-3.88 (m, 2H), 3.05-3.01 (m, 4H), 1.51-1.40 (m, 6H). LC/MS (ESI) m/z: 592 (M+H)⁺. RT (Method A): 1.75 min.

### Scheme 141. Synthesis of N-(2-(2-(((1H-Pyrrolo[3,2-c]pyridine-2-yl)methyl)amino)-2-oxoethyl)-3-fluoro-[1,1′-biphenyl]-4-yl)-5-phenylthiophene-2-carboxamide (Compound 128)

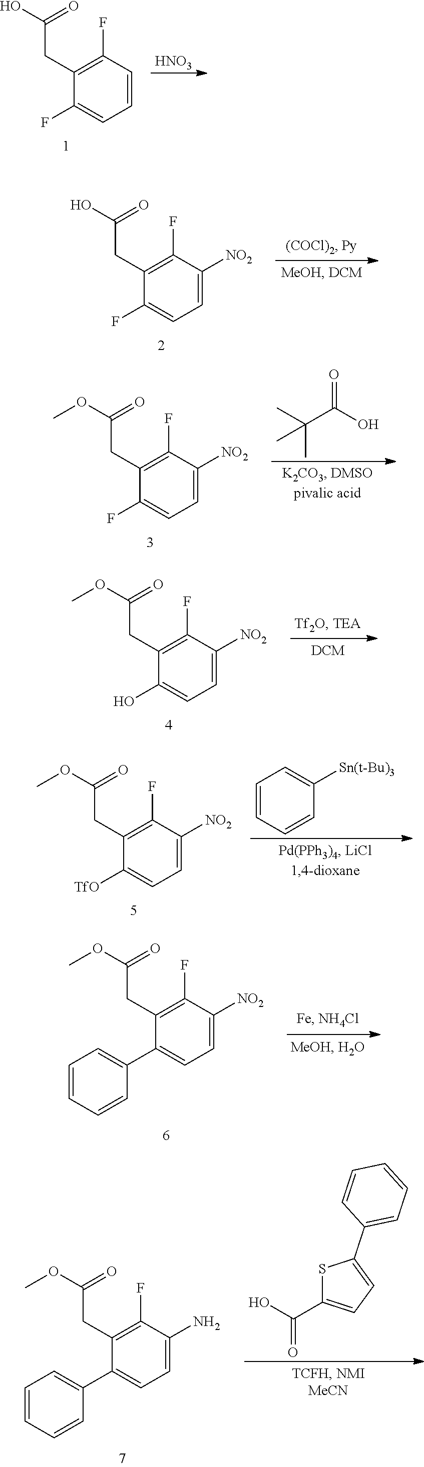

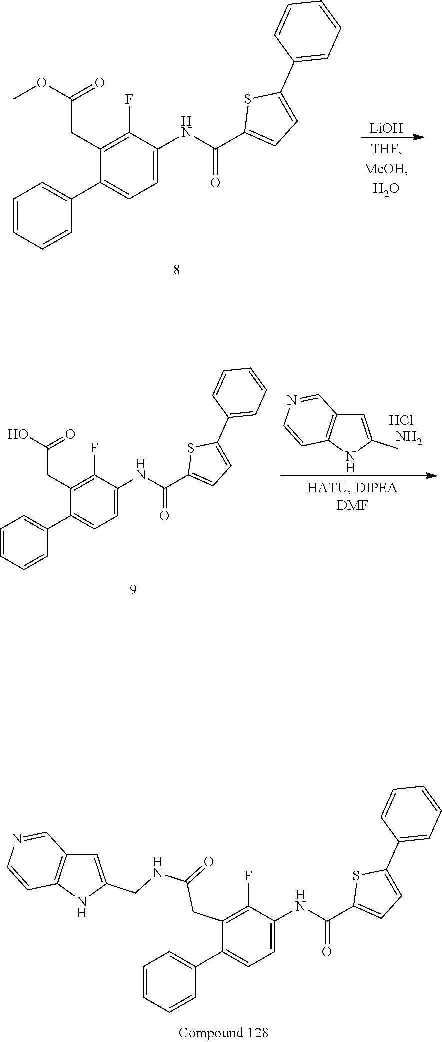

### Step 2: 2-(2,6-Difluoro-3-nitrophenyl)acetic acid (2)

2-(2,6-Difluorophenyl)acetic acid (5.0 g, 29.07 mmol) was added over 20 minutes to 50 mL of cooled fuming nitric acid at −10° C. and the reaction solution was stirred at −10° C. for 1 hour. The mixture was poured into ice and extracted with EtOAc twice. The combined organic layers were washed with brine, dried over anhydrous Na₂SO₄, filtered, and the filtrate was concentrated under reduced pressure to dryness. The residue was purified by flash chromatography (silica gel, 0-10% EtOAc in PE) to give the title compound (5.0 g, yield 79.4%) as a yellow oil. ¹H NMR (400 MHz, 00013) δ 8.13-8.00 (m, 1H), 7.13-7.02 (m, 1H), 3.75 (d, J=12.3 Hz, 2H).

### Step 2: Methyl 2-(2,6-difluoro-3-nitrophenyl)acetate (3)

To a mixture of 2-(2,6-difluoro-3-nitrophenyl)acetic acid (3.0 g, 13.80 mmol) in DCM (30 mL)/DMF (0.1 mL) was added oxalyl chloride (9.6 g, 75.59 mmol) dropwise over 30 minutes. After being stirred at room temperature for one hour, the solvent was removed to afford the acid chloride. The acid chloride was redissolved into dichloromethane (30 mL) and an excess of methanol (10 mL) was added followed by addition of pyridine (1.7 g, 22.12 mmol) under N₂ atmosphere at room temperature, the reaction mixture was degassed under N₂ atmosphere for three times and stirred at room temperature for 3 hours. The reaction mixture was diluted with EtOAc, washed with water and brine, dried over anhydrous Na₂SO₄, filtered, and the filtrate was concentrated under reduced pressure to dryness. The residue was purified by flash chromatography (silica gel, 0-30% EtOAc in PE) to give the title compound (2.6 g, yield 81.5%) as a yellow oil. ¹H NMR (400 MHz, CDCl₃) δ 8.14-8.05 (m, 1H), 7.10-7.01 (m, 1H), 3.80 (s, 2H), 3.75 (s, 3H).

### Step 3: Methyl 2-(2-fluoro-6-hydroxy-3-nitrophenyl)acetate (4)

To a mixture of methyl 2-(2,6-difluoro-3-nitrophenyl)acetate (2.6 g, 11.26 mmol) and pivalic acid (3.5 g, 34.3 mmol) in DMSO (5 mL) was added K₂CO₃ (7.6 g, 55.07 mmol) and the mixture was stirred at 80° C. for 1 hour. The mixture was acidified with 1 N aq. HCl to pH˜3 and extracted with EtOAc twice. The combined organic layers were dried over anhydrous Na₂SO₄, filtered, and concentrated under reduced pressure to dryness. The residue was purified by flash chromatography (silica gel, 0-40% EtOAc in PE) to give the title compound (800 mg, yield 31.0%) as a yellow oil. ¹H NMR (400 MHz, CDCl₃) δ 8.49 (s, 1H), 7.98 (t, J=8.9 Hz, 1H), 6.81-6.74 (m, 1H), 3.81 (s, 5H).

### Step 4: Methyl 2-(2-fluoro-3-nitro-6-(((trifluoromethyl)sulfonyl)oxy)phenyl)acetate (5)

To a solution of methyl 2-(2-fluoro-6-hydroxy-3-nitrophenyl)acetate (300 mg, 1.31 mmol) in DCM (5 mL) was added TEA (198 mg, 1.97 mmol) at −10° C. under N₂ atmosphere. After addition, triflic anhydride (580 mg, 1.78 mmol) was added to the above mixture drop-wise and the resulting mixture was stirred at −10° C. for 1 hour. The mixture was concentrated under reduced pressure to dryness. The residue was purified by flash chromatography (silica gel, 5-20% EtOAc in PE) to give the title compound (350 mg, yield 74.2%) as a yellow oil. ¹H NMR (400 MHz, CDCl₃) δ 8.11-8.04 (m, 1H), 7.28 (dd, J=9.3, 1.7 Hz, 1H), 3.80 (d, J=1.9 Hz, 2H), 3.69 (s, 3H).

### Step 5: Methyl 2-(3-fluoro-4-nitro-[1,1′-biphenyl]-2-yl)acetate (6)

To a mixture of methyl 2-(2-fluoro-3-nitro-6-(((trifluoromethyl)sulfonyl)oxy)phenyl) acetate (180 mg, 0.50 mmol) and tri-tert-butyl(phenyl)stannane (276 mg, 0.75 mmol) in 1,4-dioxane (3 mL) was added LiCl (63 mg, 1.5 mmol) and Pd(PPh₃)₄ (58 mg, 0.05 mmol) under N₂ atmosphere at room temperature, the reaction mixture was degassed under N₂ atmosphere for three times and stirred at 65° C. overnight. The reaction mixture was diluted with EtOAc, washed with water and brine, dried over anhydrous Na₂SO₄, filtered, and the filtrate was concentrated under reduced pressure to dryness. The residue was purified by flash chromatography (silica gel, 5-20% EtOAc in PE) to give the title compound (100 mg, yield 69.2%) as a yellow oil. ¹H NMR (400 MHz, CDCl₃) δ 8.00-7.94 (m, 1H), 7.42-7.37 (m, 3H), 7.23-7.19 (m, 2H), 7.18-7.15 (m, 1H), 3.64-3.61 (m, 5H).

### Step 6: Methyl 2-(4-amino-3-fluoro-[1,1′-biphenyl]-2-yl)acetate (7)

To a solution of methyl 2-(3-fluoro-4-nitro-[1,1′-biphenyl]-2-yl)acetate (100 mg, 0.35 mmol) in MeOH (2 mL) was added saturated aq. NH₄Cl solution (2 mL) and Iron (98 mg, 1.75 mmol), the mixture was degassed under N₂ atmosphere for three times and stirred at 65° C. for 1 hour. The mixture was diluted with EtOAc and filtered. The filtrate was washed with water and brine, dried over anhydrous Na₂SO₄, filtered, and concentrated under reduced pressure to dryness to give the title compound (80 mg, yield 88.7%) as a yellow oil. LC/MS (ESI) m/z: 260 (M+H)⁺.

### Step 7: Methyl 2-(3-fluoro-4-(5-phenylthiophene-2-carboxamido)-[1,1′-biphenyl]-2-yl)acetate (8)

To a mixture of methyl 2-(4-amino-3-fluoro-[1,1′-biphenyl]-2-yl)acetate (80 mg, 0.30 mmol) and 5-phenylthiophene-2-carboxylic acid (122.4 mg, 0.60 mmol) in MeCN (2 mL) was added TCFH (252 mg, 0.90 mmol) and NMI (123 mg, 1.50 mmol), the mixture was stirred at room temperature for 3 hours. The reaction mixture was diluted with EtOAc, washed with water and brine, dried over anhydrous Na₂SO₄, filtered, and concentrated under reduced pressure to dryness. The residue was purified by flash chromatography (silica gel, 5-30% EtOAc in PE) to give the title compound (65 mg, yield 48.7%) as a yellow solid. LC/MS (ESI) m/z: 446 (M+H)⁺.

### Step 8: 2-(3-Fluoro-4-(5-phenylthiophene-2-carboxamido)-[1,1′-biphenyl]-2-yl)acetic acid (9)

To a solution of methyl 2-(3-fluoro-4-(5-phenylthiophene-2-carboxamido)-[1,1′-biphenyl]-2-yl)acetate (60 mg, 0.13 mmol) in THF/MeOH/water (2.5 mL, v/v/v=2/1/2) was added LiOH (16 mg, 0.67 mmol) and the mixture was stirred at room temperature overnight. The mixture was acidified with 1 N aq. HCl to pH˜3 and extracted with EtOAc twice. The combined organic layers were dried over anhydrous Na₂SO₄, filtered, and concentrated under reduced pressure to dryness to give the title compound (40 mg, yield 71.4%) as a yellow solid, which was used directly in the next reaction without further purification. LC/MS (ESI) m/z: 432 (M+H)⁺.

### Step 9: N-(2-(2-(((1H-Pyrrolo[3,2-c]pyridine-2-yl)methyl)amino)-2-oxoethyl)-3-fluoro-[1,1′-biphenyl]-4-yl)-5-phenylthiophene-2-carboxamide (Compound 128)

To a mixture of 2-(3-fluoro-4-(5-phenylthiophene-2-carboxamido)-[1,1′-biphenyl]-2-yl)acetic acid (40 mg, 0.09 mmol) and (1H-pyrrolo[3,2-c]pyridine-2-yl)methanamine hydrochloride (32 mg, 0.18 mmol) in DMF (2 mL) was added DIPEA (35 mg, 0.27 mmol) and HATU (41 mg, 0.11 mmol) and the reaction mixture was stirred at room temperature for 1 hour. The mixture was concentrated under reduced pressure to dryness. The residue was purified by prep-HPLC (C₁₈, 40%-98% MeCN in water with 0.1% NH₃·H₂O) to give Compound 128 (10 mg, yield 19.8%) as a white solid. ¹H NMR (400 MHz, DMSO-d₆) δ 11.35 (s, 1H), 10.24 (s, 1H), 8.75 (s, 1H), 8.56 (t, J=5.6 Hz, 1H), 8.11 (d, J=5.7 Hz, 1H), 8.08 (d, J=4.0 Hz, 1H), 7.78-7.75 (m, 2H), 7.65-7.60 (m, 2H), 7.48 (t, J=7.5 Hz, 2H), 7.42-7.38 (m, 6H), 7.32 (d, J=5.7 Hz, 1H), 7.13 (d, J=8.2 Hz, 1H), 6.37 (s, 1H), 4.44 (d, J=5.5 Hz, 2H), 3.54 (s, 2H). LC/MS (ESI) m/z: 561 (M+H)⁺.

### Scheme 142. Synthesis of N-(2-(2-(((1H-Pyrrolo[3,2-c]pyridine-2-yl)methyl)amino)-2-oxoethyl)-3-methoxy-[1,1′-biphenyl]-4-yl)-5-phenylthiophene-2-carboxamide (Compound 129)

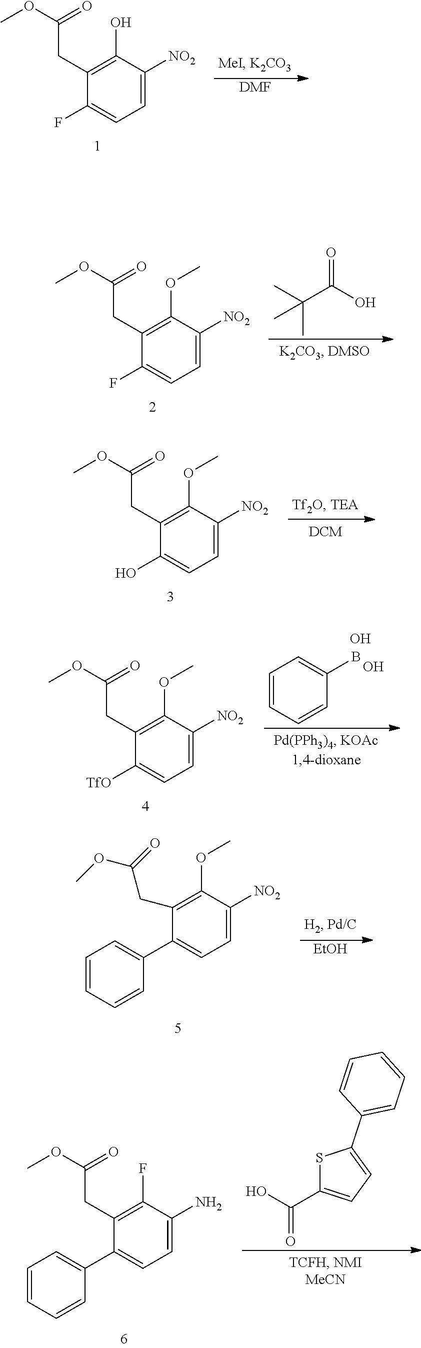

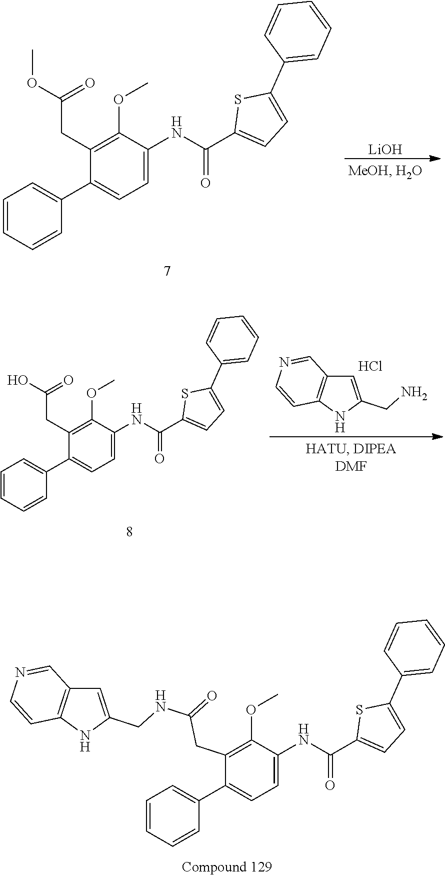

### Step 1: Methyl 2-(6-fluoro-2-methoxy-3-nitrophenyl)acetate (2)

To a mixture of methyl 2-(6-fluoro-2-hydroxy-3-nitrophenyl)acetate (1.4 g, 6.11 mmol), K₂CO₃ (2.5 g, 18.1 mmol) in DMF (20 mL) were added MeI (2.6 g, 18.3 mmol) and the mixture was stirred at 25° C. for 16 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give the title compound (1.1 g, yield 74.1%) as a yellow solid. ¹H NMR (400 MHz, CDCl₃) δ 7.91 (dd, J=9.2, 5.9 Hz, 1H), 7.01-6.94 (m, 1H), 3.91 (s, 3H), 3.77 (d, J=1.7 Hz, 2H), 3.74 (s, 3H).

### Step 2: Methyl 2-(6-hydroxy-2-methoxy-3-nitrophenyl)acetate (2)

To a mixture of methyl 2-(6-fluoro-2-methoxy-3-nitrophenyl)acetate (1.0 g, 4.11 mmol) and pivalic acid (1.33 g, 13.0 mmol) in DMSO (10 mL) was added K₂CO₃ (2.97 g, 21.5 mmol) at 0° C. and the reaction mixture was stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-40% EtOAc in PE) to give the title compound (152 mg, yield 15.3%) as a yellow solid. LC/MS (ESI) m/z: 242 (M+H)⁺.

### Step 3: Methyl 2-(2-methoxy-3-nitro-6-(((trifluoromethyl)sulfonyl)oxy)phenyl) acetate (4)

To a mixture of methyl 2-(6-hydroxy-2-methoxy-3-nitrophenyl)acetate (140 mg, 0.58 mmol) and TEA (176 mg, 1.74 mmol) in DCM (3 mL) was added triflic anhydride (580 mg, 1.78 mmol) dropwise at −20° C. and the mixture was stirred at −20° C. for 3 hours. The mixture was concentrated under reduced pressure to dryness. The residue was purified by flash chromatography (silica gel, 0-10% EtOAc in PE) to give the title compound (200 mg, yield 92.1%) as a yellow solid. ¹H NMR (400 MHz, CDCl₃) δ 7.92 (d, J=9.2 Hz, 1H), 7.26 (s, 1H), 3.93 (s, 3H), 3.84 (s, 2H), 3.75 (s, 3H).

### Step 4: Methyl 2-(3-methoxy-4-nitro-[1,1′-biphenyl]-2-yl)acetate (5)

To a mixture of methyl 2-(2-methoxy-3-nitro-6-(((trifluoromethyl)sulfonyl)oxy)phenyl) acetate (143 mg, 0.38 mmol) and phenylboronic acid (300 mg, 1.15 mmol) in 1,4-dioxane (3 mL) was added KOAc (100 mg, 1.01 mmol) and Pd(PPh₃)₄ (44 mg, 0.04 mmol) under N₂ atmosphere, the reaction mixture was degassed under N₂ atmosphere for three times and stirred at 65° C. overnight. The reaction mixture was diluted with EtOAc, washed with water and brine, dried over anhydrous Na₂SO₄, filtered, and the filtrate was concentrated under reduced pressure to dryness. The residue was purified by flash chromatography (silica gel, 0-30% EtOAc in PE) to give the title compound (70 mg, yield 61.0%) as a yellow solid. LC/MS (ESI) m/z: 320 (M+H₂O+H)⁺.

### Step 5: Methyl 2-(4-amino-3-methoxy-[1,1′-biphenyl]-2-yl)acetate (6)

To a solution of methyl 2-(3-methoxy-4-nitro-[1,1′-biphenyl]-2-yl)acetate (50 mg, 0.16 mmol) in EtOH (3 mL) was added Pd/C (10 mg, 10% wt.), the mixture was degassed under N₂ atmosphere for three times and stirred under a H₂ balloon at 25° C. overnight. The mixture was filtered, and the filtrate was concentrated to dryness to give the title compound (45 mg, yield 99.9%) as a yellow solid, which was used directly in the next step without further purification. LC/MS (ESI) m/z: 272 (M+H)⁺.

### Step 6: Methyl 2-(3-methoxy-4-(5-phenylthiophene-2-carboxamido)-[1,1′-biphenyl]-2-yl)acetate (7)

To a mixture of methyl 2-(4-amino-3-methoxy-[1,1′-biphenyl]-2-yl)acetate (40 mg, 0.14 mmol) and 5-phenylthiophene-2-carboxylic acid (45 mg, 0.22 mmol) in MeCN (3 mL) was added TCFH (123 mg, 0.22 mmol) and NMI (36 mg, 0.44 mmol), the mixture was stirred at room temperature for 18 hours. The reaction mixture was diluted with EtOAc, washed with water and brine, dried over anhydrous Na₂SO₄, filtered, and the filtrate was concentrated under reduced pressure to dryness. The residue was purified by flash chromatography (silica gel, 0-80% EtOAc in PE) to give the title compound (56 mg, yield 83.1%) as a yellow solid. LC/MS (ESI) m/z: 458 (M+H)⁺.

### Step 7: 2-(3-Methoxy-4-(5-phenylthiophene-2-carboxamido)-[1,1′-biphenyl]-2-yl) acetic acid (9)

To a solution of methyl 2-(3-methoxy-4-(5-phenylthiophene-2-carboxamido)-[1,1′-biphenyl]-2-yl)acetate (56 mg, 0.12 mmol) in THF/MeOH/water (5 mL, v/v/v=2/2/1) was added LiOH (26 mg, 1.1 mmol) and the mixture was stirred at room temperature overnight. The mixture was acidified with 1N aq. HCl to pH˜3 and extracted with EtOAc twice. The combined organic layers were dried over anhydrous Na₂SO₄, filtered, and concentrated under reduced pressure to dryness to give the title compound (48 mg, yield 88.2%) as a yellow solid, which was used directly in the next reaction without further purification. LC/MS (ESI) m/z: 444 (M+H)⁺.

### Step 8: N-(2-(2-(((1H-Pyrrolo[3,2-c]pyridine-2-yl)methyl)amino)-2-oxoethyl)-3-methoxy-[1,1′-biphenyl]-4-yl)-5-phenylthiophene-2-carboxamide (Compound 129)

To a mixture of 2-(3-methoxy-4-(5-phenylthiophene-2-carboxamido)-[1,1′-biphenyl]-2-yl)acetic acid (40 mg, 0.09 mmol) and (1H-pyrrolo[3,2-c]pyridine-2-yl)methanamine hydrochloride (25 mg, 0.13 mmol) in DMF (3 mL) was added DIPEA (58 mg, 0.45 mmol) and HATU (51 mg, 0.13 mmol) and the reaction mixture was stirred at room temperature for 1 hour. The mixture was concentrated under reduced pressure to dryness. The residue was purified by prep-HPLC (C18, 10%-90% MeCN in water with 0.1% NH₃·H₂O) to give Compound 129 (7.0 mg, yield 13.5%) as a white solid. ¹H NMR (400 MHz, CD₃OD) δ 8.77 (d, J=0.8 Hz, 1H), 8.14 (d, J=5.8 Hz, 1H), 7.99 (d, J=4.0 Hz, 1H), 7.80 (d, J=2.0 Hz, 1H), 7.78 (d, J=2.9 Hz, 2H), 7.57 (d, J=3.9 Hz, 1H), 7.53-7.49 (m, 2H), 7.45 (s, 1H), 7.43 (d, J=1.3 Hz, 1H), 7.38-7.36 (m, 5H), 7.12 (d, J=8.3 Hz, 1H), 6.50 (s, 1H), 4.53 (s, 2H), 3.83 (s, 3H), 3.66 (s, 2H). LC/MS (ESI) m/z: 573 (M+H)⁺. RT (Method A): 1.95 min.

### Scheme 143. Synthesis of (R)—N-((1H-pyrrolo[3,2-c]pyridin-2-yl)methyl)-2-(5-((1-(dibenzo[b,d]furan-2-yl)ethyl)amino)-4-methyl-6-oxo-2-phenylpyrimidin-1(6H)-yl)acetamide (Compound 336)

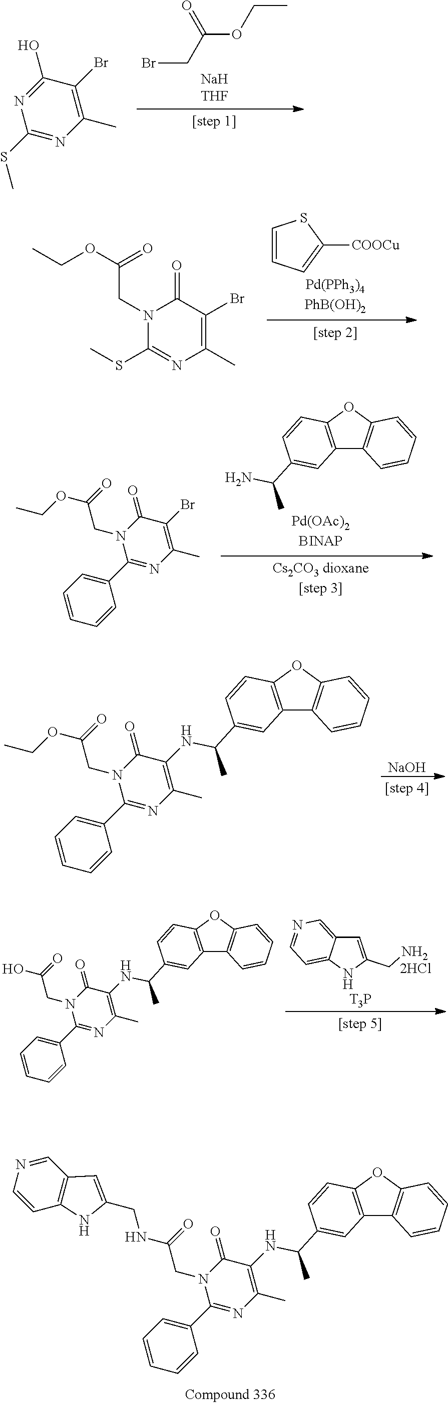

### Step 1: Ethyl 2-(5-bromo-4-methyl-2-(methylthio)-6-oxopyrimidin-1(6H)-yl)acetate

A mixture of 5-bromo-6-methyl-2-methylsulfanyl-pyrimidin-4-ol (500 mg, 2.12 mmol), CaH₂ (260 mg, 6.2 mmol), and ethyl 2-bromoacetate (0.7 mL, 6 mmol) in THF (5 mL) was stirred at room temperature for 2 hours, then heated to reflux overnight. The mixture was quenched carefully with NH₄Cl aq. and extracted by EtOAc (3×20 mL). The organic layers were washed with brine, dried over Na₂SO₄, filtered and concentrated. The residue was purified by silica gel column chromatography (0 to 40% EtOAc in hexanes) to give ethyl 2-(5-bromo-4-methyl-2-(methylthio)-6-oxopyrimidin-1(6H)-yl)acetate (300 mg, 0.93 mmol, 44% yield) as a white solid. LC/MS (ESI) m/z: 321, 323 (M+H)⁺.

### Step 2: Ethyl 2-(5-bromo-4-methyl-6-oxo-2-phenylpyrimidin-1(6H)-yl)acetate

A mixture of ethyl 2-(5-bromo-4-methyl-2-(methylthio)-6-oxopyrimidin-1(6H)-yl)acetate (0.56 g, 1.6 mmol), copper(I)-thiophene-2-carboxylate (0.46 g, 2.4 mmol), Pd(PPh₃)₄ (0.1 g, 0.09 mmol), and phenylboronic acid (0.29 g, 2.4 mmol) in THF (5 mL) was sparged with nitrogen for 10 minutes. The mixture was heated at 60° C. for 18 hours. The reaction mixture was diluted with EtOAc (10 mL) and filtered through a pad of Celite. The filtrate was concentrated and purified by silica gel column chromatography (0 to 60% EtOAc in hexanes) to give ethyl 2-(5-bromo-4-methyl-6-oxo-2-phenylpyrimidin-1(6H)-yl)acetate (420 mg, 62% yield) as a white solid. LC/MS (ESI) m/z: 351, 353 (M+H)⁺.

### Step 3: Ethyl (R)-2-(5-((1-(dibenzo[b,d]furan-2-yl)ethyl)amino)-4-methyl-6-oxo-2-phenylpyrimidin-1(6H)-yl)acetate

A mixture of ethyl 2-(5-bromo-4-methyl-6-oxo-2-phenylpyrimidin-1(6H)-yl)acetate (120 mg, 0.32 mmol), (1R)-1-dibenzofuran-2-ylethanamine (100 mg, 0.47 mmol), Pd(OAc)₂ (7.1 mg, 0.032 mmol), BINAP (40 mg, 0.064 mmol), and Cs₂CO₃ (310 mg, 0.95 mmol) in toluene (5 mL) was sparged with nitrogen for 10 minutes, then heated to 110° C. for 3 hours. The reaction was cooled down to room temperature and stirred overnight. Diluted with EtOAc (10 mL), the solid was removed by filtration over a Celite pad. The filtrate was concentrated and purified on silica gel column chromatography (0 to 5% MeOH in DCM) to give Ethyl (R)-2-(5-((1-(dibenzo[b,d]furan-2-yl)ethyl)amino)-4-methyl-6-oxo-2-phenylpyrimidin-1(6H)-yl)acetate (200 mg, 87% yield) as a light brown oil. LC/MS (ESI) m/z=482 (M+H)⁺.

### Step 4: (R)-2-(5-((1-(dibenzo[b,d]furan-2-yl)ethyl)amino)-4-methyl-6-oxo-2-phenylpyrimidin-1(6H)-yl)acetic acid

To a suspension of Ethyl (R)-2-(5-((1-(dibenzo[b,d]furan-2-yl)ethyl)amino)-4-methyl-6-oxo-2-phenylpyrimidin-1(6H)-yl)acetate (160 mg, 0.31 mmol) in a 1:1:1 mixture of MeOH/THF/H₂O (6 mL). The reaction was heated to 55° C. for 4 hours. After cooling down to room temperature, the solution was diluted with water and concentrated. The mixture was extracted with EtOAc (2×10 mL). Organic layers were discarded. The aqueous layer was acidified with 1 N HCl aq. to pH 3-4 and extracted with EtOAc (3×20 mL). The organic layer was washed with brine, dried over Na₂SO₄, filtered, and concentrated to give (R)-2-(5-((1-(dibenzo[b,d]furan-2-yl)ethyl)amino)-4-methyl-6-oxo-2-phenylpyrimidin-1(6H)-yl)acetic acid (100 mg, 70% yield) as a light yellow foamy solid. LC/MS (ESI) m/z: 454 (M+H)⁺.

### Step 5: (R)—N-((1H-pyrrolo[3,2-c]pyridin-2-yl)methyl)-2-(5-((1-(dibenzo[b,d]furan-2-yl)ethyl)amino)-4-methyl-6-oxo-2-phenylpyrimidin-1(6H)-yl)acetamide (Compound 336)

To an ice-bath cooled suspension of (R)-2-(5-((1-(dibenzo[b,d]furan-2-yl)ethyl)amino)-4-methyl-6-oxo-2-phenylpyrimidin-1(6H)-yl)acetic acid (100 mg, 0.22 mmol), 1H-pyrrolo[3,2-c]305 yridine-2-yl)methanamine dihydrochloride (51 mg, 0.23 mmol), 50% T₃P in EtOAc (0.28 mL, 0.94 mmol) in DMF (3 mL) was added DIPEA (0.2 mL, 1 mmol). The reaction was stirred for 40 minutes and quenched with saturated NaHCO₃ aq. The solid was filtered and the filter cake was dissolved in DMSO (3 mL), which was purified by an ACCQ Prep-HPLC (0 to 100% MeCN in H₂O (containing 0.05% HCOOH)) to give Compound 336 (47 mg, 37% yield) as an off-white powder. ¹H-NMR (400 MHz, DMSO-d₆) δ 11.35 (s, 1H), 8.74 (s, 1H), 8.69 (t, J=5.8 Hz, 1H), 8.23-7.99 (m, 3H), 7.67 (d, J=8.3 Hz, 1H), 7.61 (d, J=8.5 Hz, 1H), 7.57-7.29 (m, 9H), 6.35 (s, 1H), 5.03 (s, 1H), 4.83 (d, J=10.1 Hz, 1H), 4.55-4.38 (m, 4H), 2.25 (s, 3H), 1.55 (d, J=6.7 Hz, 3H). LC/MS (ESI) m/z: 583 (M+H)⁺. RT (Method A): 1.64 min.

Compound 337 was prepared based on Steps 3-5 in Scheme 143.

| # | Reactant A | Reactant D | Reactant E | Characterization Data |
|---|---|---|---|---|
| 337^{a} | 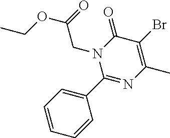 | 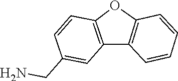 [SMILES: NCc1ccc2oc3ccccc3c2c1] | 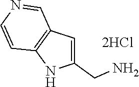 [SMILES: NCc1cc2cnccc2[nH]1 ; "2HCl"] | ¹H-NMR (400 MHz, DMSO-d₆) δ 11.26 (s, 1H), 8.73-8.49 (m, 2H), 8.09-7.84 (m, 3H), 7.59 (dd, J = 16.8, 8.4 Hz, 2H), 7.50-7.14 (m, 8H), 6.28 (s, 1H), 5.10 (s, 1H), 4.54 (d, J = 6.1 Hz, 2H), 4.48-4.27 (m, 2H), 2.21 (s, 3H). LC/MS (ESI) m/z: 569 (M + H)⁺. RT (Method A): 1.53 min. |

### Example 2. Non-Limiting Examples of Compounds of the Present Disclosure

Table 1 shows illustrative complement pathway inhibitors.

TABLE 1
Non-limiting Examples of Compounds of the Present Disclosure

| # | Structure | Name |
|---|---|---|
| 1 | 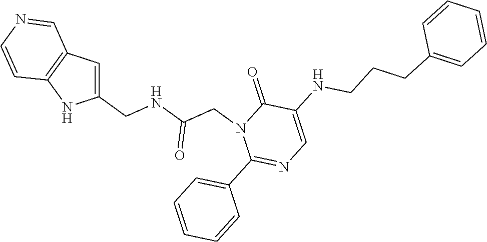 [SMILES: O=C(Cn1c(-c2ccccc2)ncc(NCCCc2ccccc2)c1=O)NCc1cc2cnccc2[nH]1] | N-((1H-pyrrolo[3,2-c]pyridin- 2-yl)methyl)-2-(6-oxo-2-phen- yl-5-((3-phenylpropyl)amino)- pyrimidin-1(6H)-yl)acetamide |
| 2 | 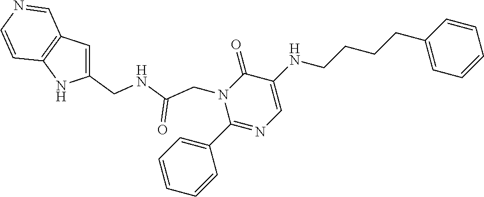 [SMILES: O=C(Cn1c(-c2ccccc2)ncc(NCCCCc2ccccc2)c1=O)NCc1cc2cnccc2[nH]1] | N-((1H-pyrrolo[3,2-c]pyridin- 2-yl)methyl)-2-(6-oxo-2-phen- yl-5-((4-phenylbutyl)amino)- pyrimidin-1(6H)-yl)acetamide |
| 3 | 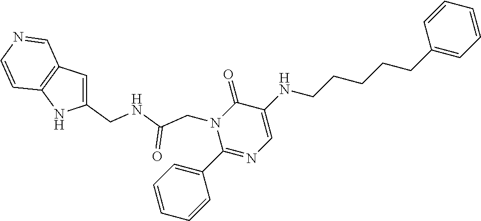 [SMILES: O=C(Cn1c(-c2ccccc2)ncc(NCCCCCc2ccccc2)c1=O)NCc1cc2cnccc2[nH]1] | N-((1H-pyrrolo[3,2-c]pyridin- 2-yl)methyl)-2-(6-oxo-2-phen- yl-5-((5-phenylpentyl)amino)- pyrimidin-1(6H)-yl)acetamide |
| 4 | 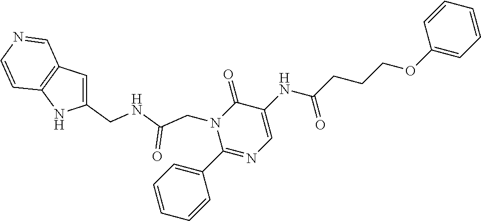 [SMILES: O=C(Cn1c(-c2ccccc2)ncc(NC(=O)CCCOc2ccccc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-phen- oxybutanamide |
| 5 | 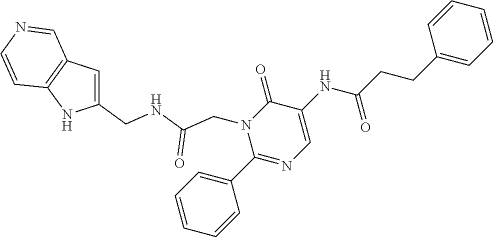 [SMILES: O=C(Cn1c(-c2ccccc2)ncc(NC(=O)CCc2ccccc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-3-phen- ylpropanamide |
| 6 | 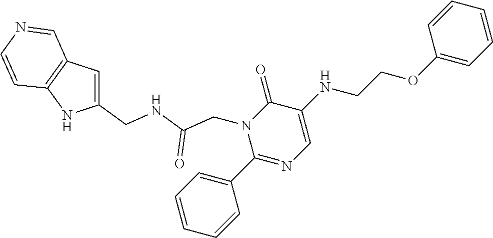 [SMILES: O=C(Cn1c(-c2ccccc2)ncc(NCCOc2ccccc2)c1=O)NCc1cc2cnccc2[nH]1] | N-((1H-pyrrolo[3,2-c]pyridin- 2-yl)methyl)-2-(6-oxo-5-((2- phenoxyethyl)amino)-2-phen- ylpyrimidin-1(6H)-yl)aceamide |
| 7 | 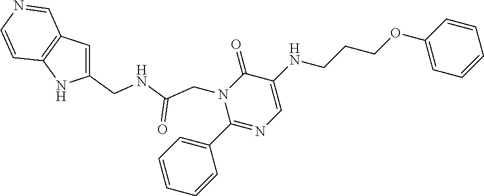 [SMILES: O=C(Cn1c(-c2ccccc2)ncc(NCCCOc2ccccc2)c1=O)NCc1cc2cnccc2[nH]1] | N-((1H-pyrrolo[3,2-c]pyridin- 2-yl)methyl)-2-(6-oxo-5-((3- phenoxypropyl)amino)-2- phenylpyrimidin-1(6H)-yl)- acetamide |
| 8 | 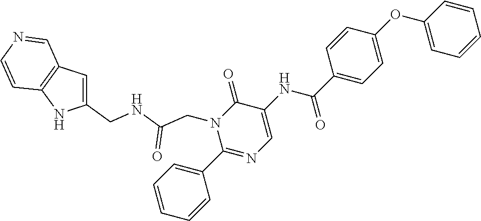 [SMILES: O=C(Cn1c(-c2ccccc2)ncc(NC(=O)c2ccc(Oc3ccccc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-phen- oxybenzamide |
| 9 | 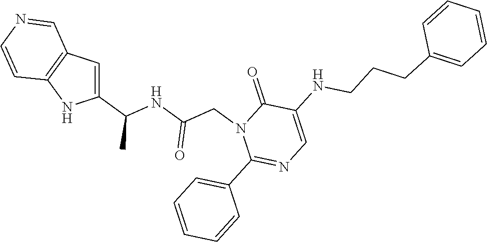 [SMILES: C[C@H](NC(=O)Cn1c(-c2ccccc2)ncc(NCCCc2ccccc2)c1=O)c1cc2cnccc2[nH]1] | (S)-N-(1-(1H-pyrrolo[3,2-c] pyridin-2-yl)ethyl)-2-(6-oxo- 2-phenyl-5-((3-phenylpropyl)- amino)pyrimidin-1(6H)-yl)- acetamide |
| 10 | 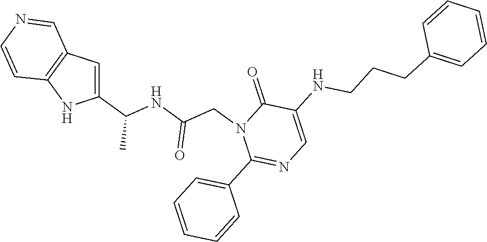 [SMILES: C[C@@H](NC(=O)Cn1c(-c2ccccc2)ncc(NCCCc2ccccc2)c1=O)c1cc2cnccc2[nH]1] | (R)-N-(1-(1H-pyrrolo[3,2-c] pyridin-2-yl)ethyl)-2-(6-oxo- 2-phenyl-5-((3-phenylpropyl)- amino)pyrimidin-1(6H)-yl)- acetamide |
| 11 | 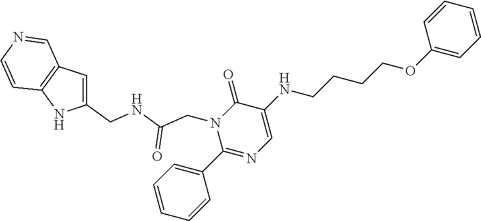 [SMILES: O=C(Cn1c(-c2ccccc2)ncc(NCCCCOc2ccccc2)c1=O)NCc1cc2cnccc2[nH]1] | N-((1H-pyrrolo[3,2-c]pyridin- 2-yl)methyl)-2-(6-oxo-5-((4- phenoxybutyl)amino)-2- phenylpyrimidin-1(6H)-yl)- acetamide |
| 12 | 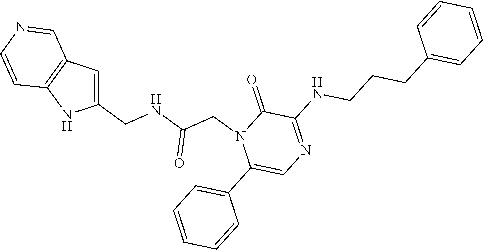 [SMILES: O=C(Cn1c(-c2ccccc2)cnc(NCCCc2ccccc2)c1=O)NCc1cc2cnccc2[nH]1] | (R)-N-(1-(1H-pyrrolo[3,2-c] pyridin-2-yl)ethyl)-2-(2-oxo- 6-phenyl-3-((3-phenylpropyl)- amino)pyrazin-1(2H)-yl)- acetamide |
| 13 | 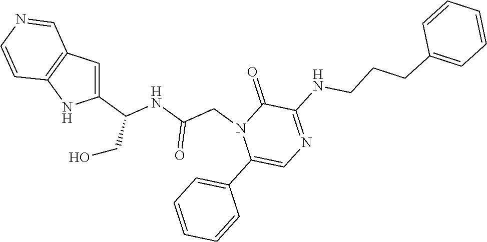 [SMILES: O=C(Cn1c(-c2ccccc2)cnc(NCCCc2ccccc2)c1=O)N[C@H](CO)c1cc2cnccc2[nH]1] | (S)-N-(2-hydroxy-1-(1H-pyr- rolo[3,2-c]pyridin-2-yl)ethyl)- 2-(2-oxo-6-phenyl-3-((3-phen- ylpropyl)amino)pyrazin-1(2H)- yl)acetamide |
| 14 | 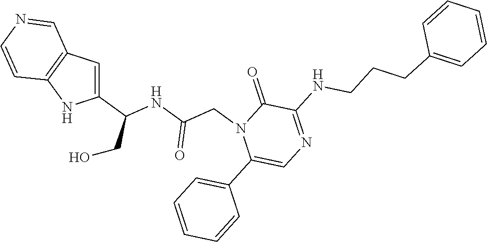 [SMILES: O=C(Cn1c(-c2ccccc2)cnc(NCCCc2ccccc2)c1=O)N[C@@H](CO)c1cc2cnccc2[nH]1] | (R)-N-(2-hydroxy-1-(1H-pyr- rolo[3,2-c]pyridin-2-yl)ethyl)- 2-(2-oxo-6-phenyl-3-((3-phen- ylpropyl)amino)pyrazin-1(2H)- yl)acetamide |
| 15 | 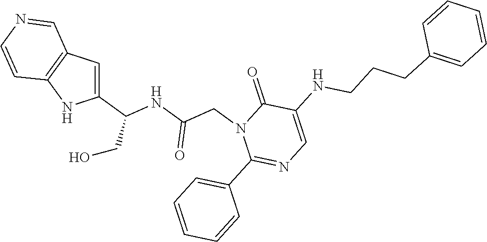 [SMILES: O=C(Cn1c(-c2ccccc2)ncc(NCCCc2ccccc2)c1=O)N[C@H](CO)c1cc2cnccc2[nH]1] | (S)-N-(2-hydroxy-1-(1H-pyr- rolo[3,2-c]pyridin-2-yl)ethyl)- 2-(6-oxo-2-phenyl-5-((3-phen- ylpropyl)amino)pyrimidin- 1(6H)-yl)acetamide |
| 16 | 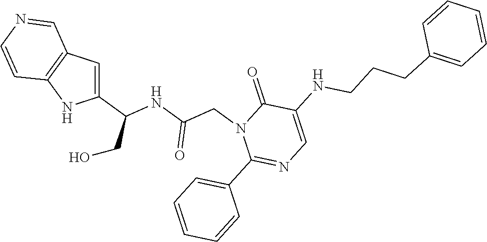 [SMILES: O=C(Cn1c(-c2ccccc2)ncc(NCCCc2ccccc2)c1=O)N[C@@H](CO)c1cc2cnccc2[nH]1] | (R)-N-(2-hydroxy-1-(1H-pyr- rolo[3,2-c]pyridin-2-yl)ethyl)- 2-(6-oxo-2-phenyl-5-((3-phen- ylpropyl)amino)pyrimidin- 1(6H)-yl)acetamide |
| 17 | 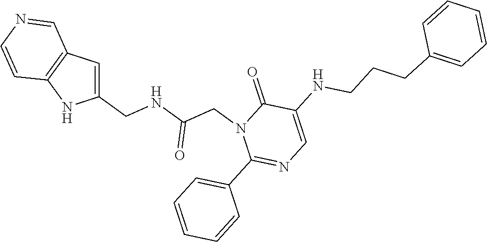 [SMILES: O=C(Cn1c(-c2ccccc2)ncc(NCCCc2ccccc2)c1=O)NCc1cc2cnccc2[nH]1] | N-((1H-pyrrolo[3,2-c]pyridin- 2-yl)methyl)-2-(2-oxo-6-phen- yl-3-((3-phenylpropyl)amino)- pyrazin-1(2H)-yl)acetamide |
| 18 | 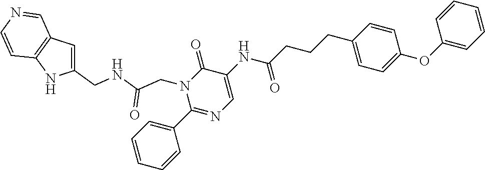 [SMILES: O=C(Cn1c(-c2ccccc2)ncc(NC(=O)CCCc2ccc(Oc3ccccc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(4- phenoxyphenyl)butanamide |
| 19 | 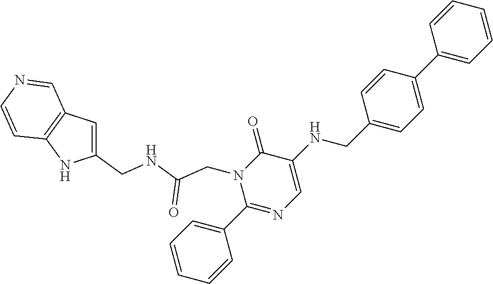 [SMILES: O=C(Cn1c(-c2ccccc2)ncc(NCc2ccc(-c3ccccc3)cc2)c1=O)NCc1cc2cnccc2[nH]1] | N-((1H-pyrrolo[3,2-c]pyridin- 2-yl)methyl)-2-(5-(([1,1′- biphenyl]-4-ylmethyl)amino)- 6-oxo-2-phenylpyrimidin- 1(6H)-yl)acetamide |
| 20 | 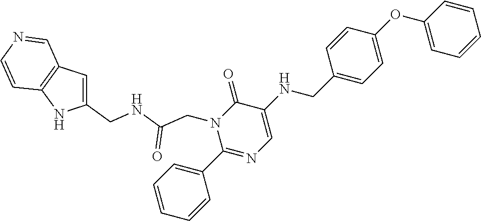 [SMILES: O=C(Cn1c(-c2ccccc2)ncc(NCc2ccc(Oc3ccccc3)cc2)c1=O)NCc1cc2cnccc2[nH]1] | N-((1H-pyrrolo[3,2-c]pyridin- 2-yl)methyl)-2-(6-oxo-5-((4- phenoxybenzyl)amino)-2- phenylpyrimidin-1(6H)-yl)- acetamide |
| 21 | 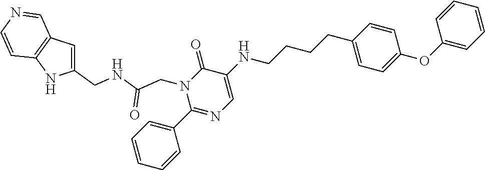 [SMILES: O=C(Cn1c(-c2ccccc2)ncc(NCCCCc2ccc(Oc3ccccc3)cc2)c1=O)NCc1cc2cnccc2[nH]1] | N-((1H-pyrrolo[3,2-c]pyridin- 2-yl)methyl)-2-(6-oxo-5-((4- (4-phenoxyphenyl)butyl)- amino)-2-phenylpyrimidin- 1(6H)-yl)acetamide |
| 22 | 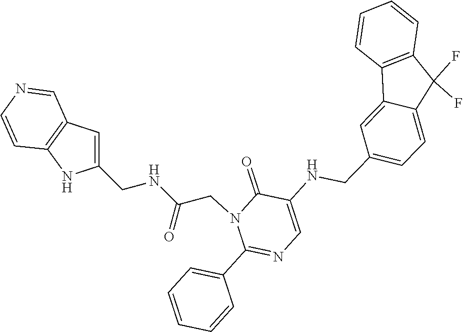 [SMILES: O=C(Cn1c(-c2ccccc2)ncc(NCc2ccc3c(c2)-c2ccccc2C3(F)F)c1=O)NCc1cc2cnccc2[nH]1] | N-((1H-pyrrolo[3,2-c]pyridin- 2-yl)methyl)-2-(5-(((9,9- difluoro-9H-fluoren-3-yl)meth- yl)amino)-6-oxo-2-phenyl- pyrimidin-1(6H)-yl)acetamide |
| 23 | 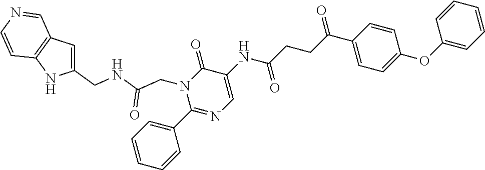 [SMILES: O=C(Cn1c(-c2ccccc2)ncc(NC(=O)CCC(=O)c2ccc(Oc3ccccc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-oxo- 4-(4-phenoxyphenyl)butan- amide |
| 24 | 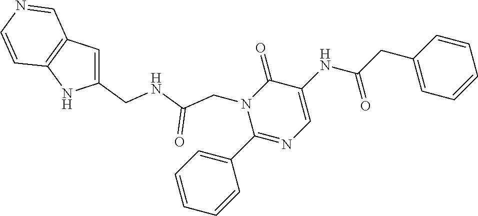 [SMILES: O=C(Cn1c(-c2ccccc2)ncc(NC(=O)Cc2ccccc2)c1=O)NCc1cc2cnccc2[nH]1] | N-((1H-pyrrolo[3,2-c]pyridin- 2-yl)methyl)-2-(6-oxo-2-phen- yl-5-(2-phenylacetamido)pyr- imidin-1(6H)-yl)acetamide |
| 25 | 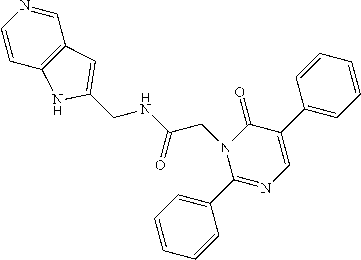 [SMILES: O=C(Cn1c(-c2ccccc2)ncc(-c2ccccc2)c1=O)NCc1cc2cnccc2[nH]1] | N-((1H-pyrrolo[3,2-c]pyridin- 2-yl)methyl)-2-(6-oxo-2,5- diphenylpyrimidin-1(6H)-yl)- acetamide |
| 26 | 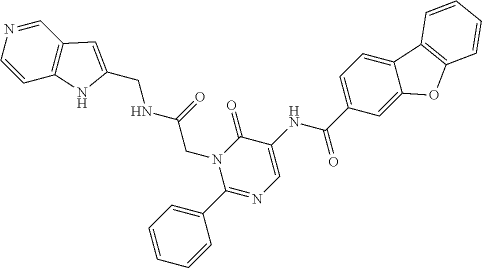 [SMILES: O=C(Cn1c(-c2ccccc2)ncc(NC(=O)c2ccc3c(c2)oc2ccccc23)c1=O)NCc1cc2cnccc2[nH]1] | N-((1H-pyrrolo[3,2-c]pyridin- 2-yl)methyl)-2-(5-((dibenzo [b,d]furan-3-ylmethyl)amino)- 6-oxo-2-phenylpyrimidin- 1(6H)-yl)acetamide |
| 27 | 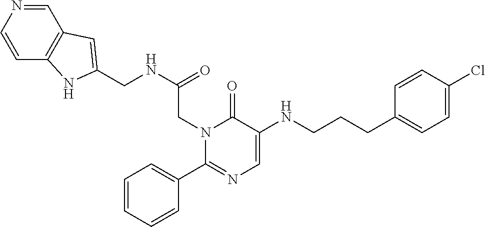 [SMILES: O=C(Cn1c(-c2ccccc2)ncc(NCCCc2ccc(Cl)cc2)c1=O)NCc1cc2cnccc2[nH]1] | N-((1H-pyrrolo[3,2-c]pyridin- 2-yl)methyl)-2-(5-((3-(4- chlorophenyl)propyl)amino)- 6-oxo-2-phenylpyrimidin- 1(6H)-yl)acetamide |
| 28 | 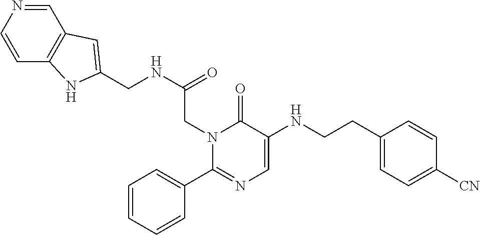 [SMILES: N#Cc1ccc(CCNc2cnc(-c3ccccc3)n(CC(=O)NCc3cc4cnccc4[nH]3)c2=O)cc1] | N-((1H-pyrrolo[3,2-c]pyridin- 2-yl)methyl)-2-(5-((4-cyano- phenethyl)amino)-6-oxo-2- phenylpyrimidin-1(6H)-yl)- acetamide |
| 29 | 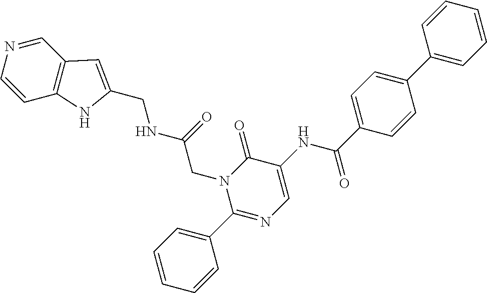 [SMILES: O=C(Cn1c(-c2ccccc2)ncc(NC(=O)c2ccc(-c3ccccc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-[1,1′- biphenyl]-4-carboxamide |
| 30 | 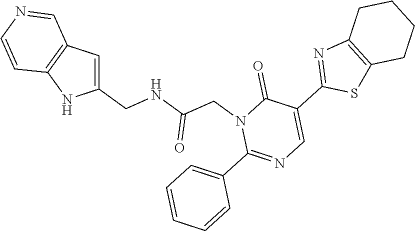 [SMILES: O=C(Cn1c(-c2ccccc2)ncc(-c2nc3c(s2)CCCC3)c1=O)NCc1cc2cnccc2[nH]1] | N-((1H-pyrrolo[3,2-c]pyridin- 2-yl)methyl)-2-(6-oxo-2-phen- yl-5-(4,5,6,7-tetrahydrobenzo [d]thiazol-2-yl)pyrimidin- 1(6H)-yl)acetamide |
| 31 | 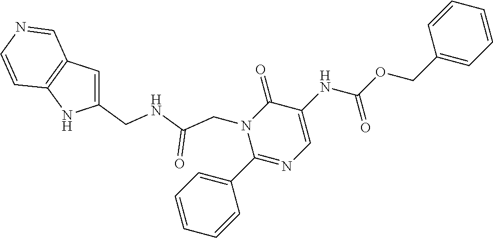 [SMILES: O=C(Cn1c(-c2ccccc2)ncc(NC(=O)OCc2ccccc2)c1=O)NCc1cc2cnccc2[nH]1] | benzyl (1-(2-(((1H-pyrrolo[3,2- c]pyridin-2-yl)methyl)amino)- 2-oxoethyl)-6-oxo-2-phenyl- 1,6-dihydropyrimidin-5-yl)- carbamate |
| 32 | 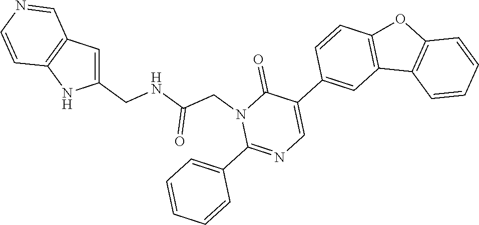 [SMILES: O=C(Cn1c(-c2ccccc2)ncc(-c2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-phenylpyrimidin- 1(6H)-yl)acetamide |
| 33 | 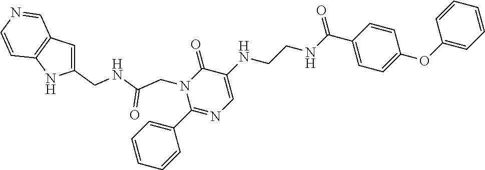 [SMILES: O=C(Cn1c(-c2ccccc2)ncc(NCCNC(=O)c2ccc(Oc3ccccc3)cc2)c1=O)NCc1cc2cnccc2[nH]1] | N-(2-((1-(2-(((1H-pyrrolo[3,2- c]pyridin-2-yl)methyl)amino)- 2-oxoethyl)-6-oxo-2-phenyl- 1,6-dihydropyrimidin-5-yl)- amino)ethyl)-4-phenoxybenz- amide |
| 34 | 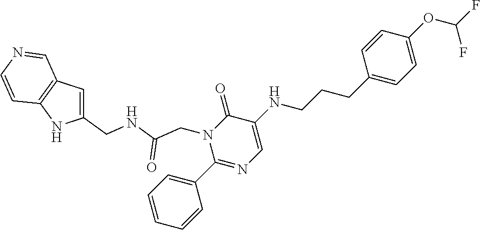 [SMILES: O=C(Cn1c(-c2ccccc2)ncc(NCCCc2ccc(OC(F)F)cc2)c1=O)NCc1cc2cnccc2[nH]1] | N-((1H-pyrrolo[3,2-c]pyridin- 2-yl)methyl)-2-(5-((3-(4- (difluoromethoxy)phenyl)prop- yl)amino)-6-oxo-2-phenyl- pyrimidin-1(6H)-yl)acetamide |
| 35 | 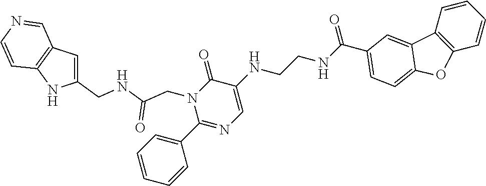 [SMILES: O=C(Cn1c(-c2ccccc2)ncc(NCCNC(=O)c2ccc3oc4ccccc4c3c2)c1=O)NCc1cc2cnccc2[nH]1] | N-(2-((1-(2-(((1H-pyrrolo[3,2- c]pyridin-2-yl)methyl)amino)- 2-oxoethyl)-6-oxo-2-phenyl- 1,6-dihydropyrimidin-5-yl)- amino)ethyl)dibenzo[b,d]furan- 2-carboxamide |
| 36 | 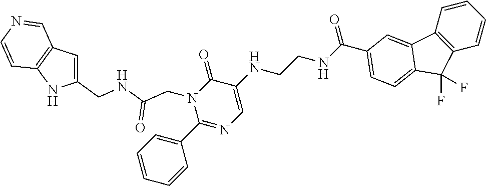 [SMILES: O=C(Cn1c(-c2ccccc2)ncc(NCCNC(=O)c2ccc3c(c2)-c2ccccc2C3(F)F)c1=O)NCc1cc2cnccc2[nH]1] | N-(2-((1-(2-(((1H-pyrrolo[3,2- c]pyridin-2-yl)methyl)amino)- 2-oxoethyl)-6-oxo-2-phenyl- 1,6-dihydropyrimidin-5-yl)- amino)ethyl)-9,9-difluoro-9H- fluorene-3-carboxamide |
| 37 | 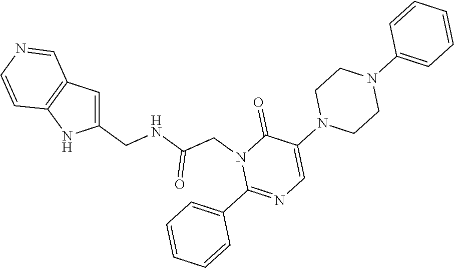 [SMILES: O=C(Cn1c(-c2ccccc2)ncc(N2CCN(c3ccccc3)CC2)c1=O)NCc1cc2cnccc2[nH]1] | N-((1H-pyrrolo[3,2-c]pyridin- 2-yl)methyl)-2-(6-oxo-2-phen- yl-5-(4-phenylpiperazin-1-yl)- pyrimidin-1(6H)-yl)acetamide |
| 38 | 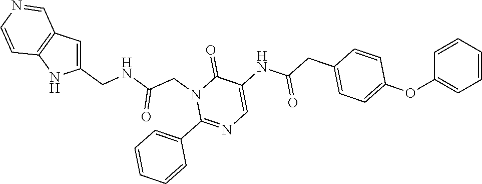 [SMILES: O=C(Cn1c(-c2ccccc2)ncc(NC(=O)Cc2ccc(Oc3ccccc3)cc2)c1=O)NCc1cc2cnccc2[nH]1] | N-((1H-pyrrolo[3,2-c]pyridin- 2-yl)methyl)-2-(6-oxo-5-(2-(4- phenoxyphenyl)acetamido)-2- phenylpyrimidin-1(6H)-yl)- acetamide |
| 39 | 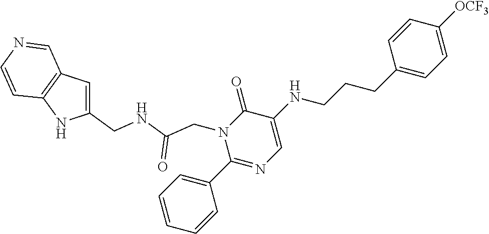 [SMILES: O=C(Cn1c(-c2ccccc2)ncc(NCCCc2ccc(OC(F)(F)F)cc2)c1=O)NCc1cc2cnccc2[nH]1] | N-((1H-pyrrolo[3,2-c]pyridin- 2-yl)methyl)-2-(6-oxo-2-phen- yl-5-((3-(4-(trifluoromethoxy)- phenyl)propyl)amino)pyrimi- din-1(6H)-yl)acetamide |
| 40 | 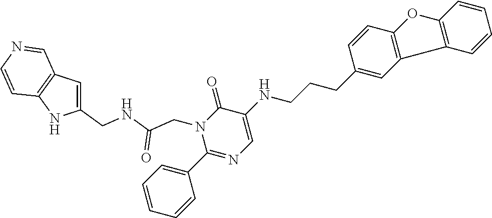 [SMILES: O=C(Cn1c(-c2ccccc2)ncc(NCCCc2ccc3oc4ccccc4c3c2)c1=O)NCc1cc2cnccc2[nH]1] | N-((1H-pyrrolo[3,2-c]pyridin- 2-yl)methyl)-2-(5-((3-(dibenzo [b,d]furan-2-yl)propyl)amino)- 6-oxo-2-phenylpyrimidin- 1(6H)-yl)acetamide |
| 41 | 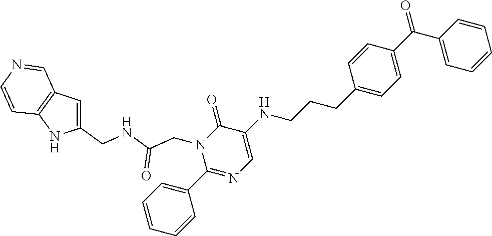 [SMILES: O=C(Cn1c(-c2ccccc2)ncc(NCCCc2ccc(C(=O)c3ccccc3)cc2)c1=O)NCc1cc2cnccc2[nH]1] | N-((1H-pyrrolo[3,2-c]pyridin- 2-yl)methyl)-2-(5-((3-(4-benzo- ylphenyl)propyl)amino)-6-oxo- 2-phenylpyrimidin-1(6H)-yl)- acetamide |
| 42 | 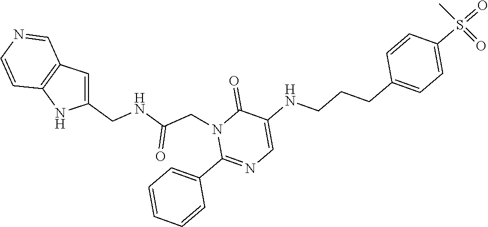 [SMILES: CS(=O)(=O)c1ccc(CCCNc2cnc(-c3ccccc3)n(CC(=O)NCc3cc4cnccc4[nH]3)c2=O)cc1] | N-((1H-pyrrolo[3,2-c]pyridin- 2-yl)methyl)-2-(5-((3-(4- (methylsulfonyl)phenyl)prop- yl)amino)-6-oxo-2-phenylpyr- imidin-1(6H)-yl)acetamide |
| 43 | 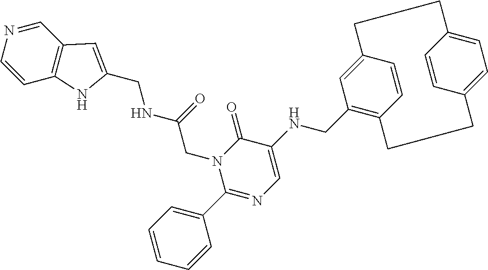 [SMILES: O=C(Cn1c(-c2ccccc2)ncc(NCc2cc3ccc2CCc2ccc(cc2)CC3)c1=O)NCc1cc2cnccc2[nH]1] | 2-(5-((1,4(1,4)-dibenzena- cyclohexaphane-1(2)-ylmeth- yl)amino)-6-oxo-2-phenylpyr- imidin-1(6H)-yl)-N-((1H-pyr- rolo[3,2-c]pyridin-2-yl)meth- yl)acetamide |
| 44 | 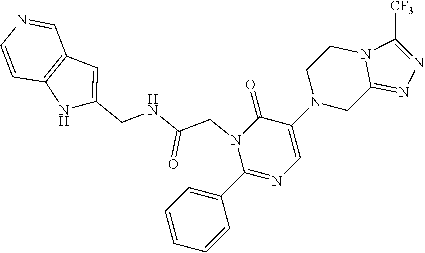 [SMILES: O=C(Cn1c(-c2ccccc2)ncc(N2CCn3c(nnc3C(F)(F)F)C2)c1=O)NCc1cc2cnccc2[nH]1] | N-((1H-pyrrolo[3,2-c]pyridin- 2-yl)methyl)-2-(6-oxo-2-phen- yl-5-(3-(trifluoromethyl)-5,6- dihydro-[1,2,4]triazolo[4,3-a] pyrazin-7(8H)-yl)pyrimidin- 1(6H)-yl)acetamide |
| 45 | 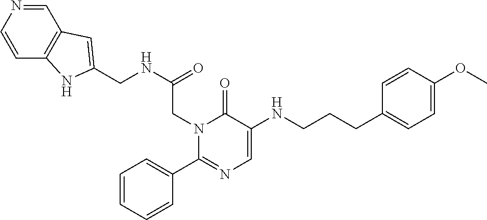 [SMILES: COc1ccc(CCCNc2cnc(-c3ccccc3)n(CC(=O)NCc3cc4cnccc4[nH]3)c2=O)cc1] | N-((1H-pyrrolo[3,2-c]pyridin- 2-yl)methyl)-2-(5-((3-(4-meth- oxyphenyl)propyl)amino)-6- oxo-2-phenylpyrimidin-1(6H)- yl)acetamide |
| 46 | 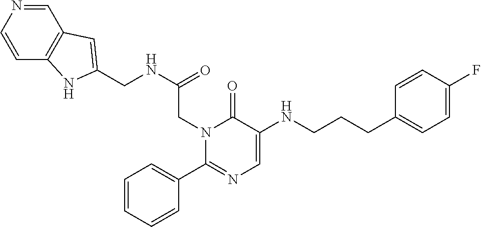 [SMILES: O=C(Cn1c(-c2ccccc2)ncc(NCCCc2ccc(F)cc2)c1=O)NCc1cc2cnccc2[nH]1] | N-((1H-pyrrolo[3,2-c]pyridin- 2-yl)methyl)-2-(5-((3-(4-fluoro- phenyl)propyl)amino)-6-oxo-2- phenylpyrimidin-1(6H)-yl)- acetamide |
| 47 | 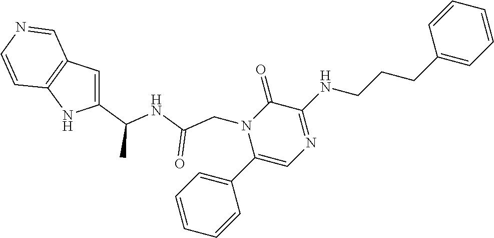 [SMILES: C[C@H](NC(=O)Cn1c(-c2ccccc2)cnc(NCCCc2ccccc2)c1=O)c1cc2cnccc2[nH]1] | (S)-N-(1-(1H-pyrrolo[3,2-c] pyridin-2-yl)ethyl)-2-(2-oxo- 6-phenyl-3-((3-phenylpropyl)- amino)pyrazin-1(2H)-yl)- acetamide |
| 48 | 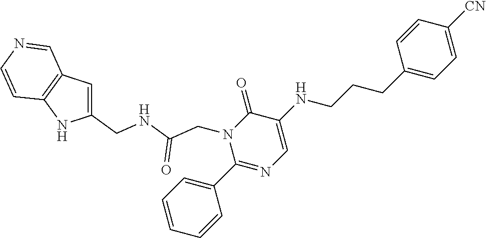 [SMILES: N#Cc1ccc(CCCNc2cnc(-c3ccccc3)n(CC(=O)NCc3cc4cnccc4[nH]3)c2=O)cc1] | N-((1H-pyrrolo[3,2-c]pyridin- 2-yl)methyl)-2-(5-((3-(4-cyano- phenyl)propyl)amino)-6-oxo-2- phenylpyrimidin-1(6H)-yl)- acetamide |
| 49 | 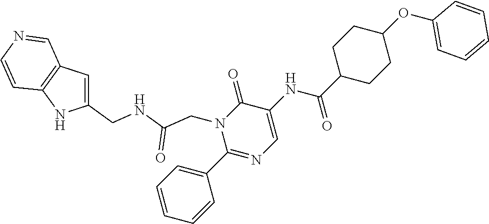 [SMILES: O=C(Cn1c(-c2ccccc2)ncc(NC(=O)C2CCC(Oc3ccccc3)CC2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-phen- oxycyclohexane-1-carboxamide |
| 50 | 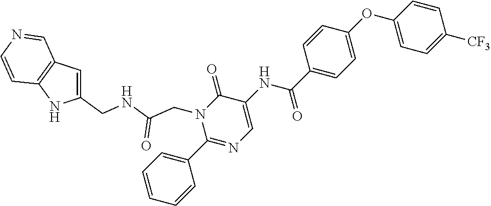 [SMILES: O=C(Cn1c(-c2ccccc2)ncc(NC(=O)c2ccc(Oc3ccc(C(F)(F)F)cc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(4- (trifluoromethyl)phenoxy)- benzamide |
| 51 | 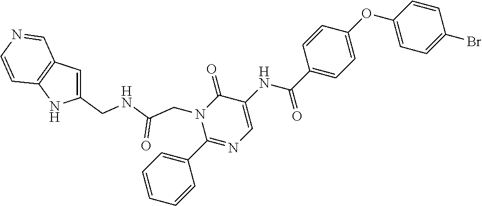 [SMILES: O=C(Cn1c(-c2ccccc2)ncc(NC(=O)c2ccc(Oc3ccc(Br)cc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(4- bromophenoxy)benzamide |
| 52 | 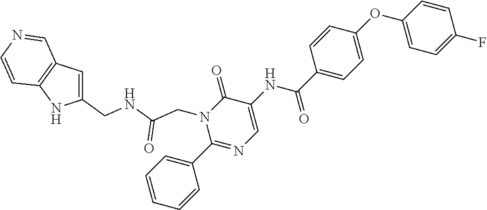 [SMILES: O=C(Cn1c(-c2ccccc2)ncc(NC(=O)c2ccc(Oc3ccc(F)cc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(4- fluorophenoxy)benzamide |
| 53 | 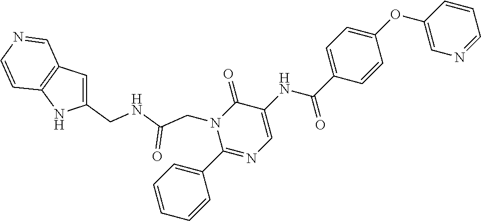 [SMILES: O=C(Cn1c(-c2ccccc2)ncc(NC(=O)c2ccc(Oc3cccnc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4- (pyridin-3-yloxy)benzamide |
| 54 | 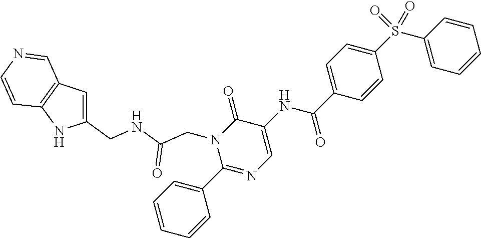 [SMILES: O=C(Cn1c(-c2ccccc2)ncc(NC(=O)c2ccc(S(=O)(=O)c3ccccc3)cc2)c1=O)NCc1cc2cnccc2[nH]1] | 4-(benzenesulfonyl)-N-[6-oxo- 1-[2-oxo-2-(1H-pyrrolo[3,2-c] pyridin-2-ylmethylamino)- ethyl]-2-phenyl-pyrimidin-5- yl]benzamide |
| 55 | 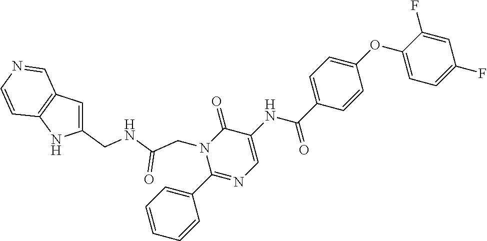 [SMILES: O=C(Cn1c(-c2ccccc2)ncc(NC(=O)c2ccc(Oc3ccc(F)cc3F)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(2,4- difluorophenoxy)benzamide |
| 56 | 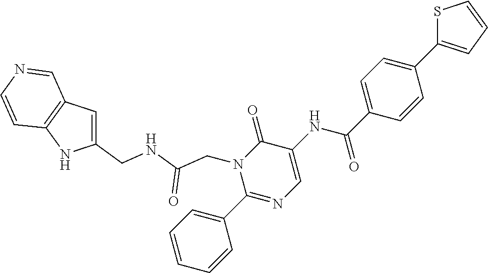 [SMILES: O=C(Cn1c(-c2ccccc2)ncc(NC(=O)c2ccc(-c3cccs3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4- (thiophen-2-yl)benzamide |
| 57 | 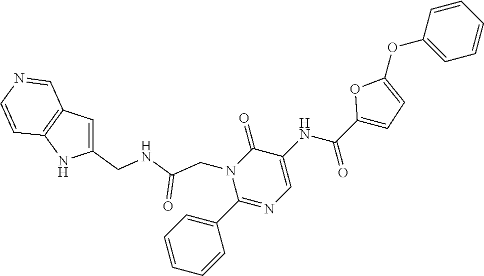 [SMILES: O=C(Cn1c(-c2ccccc2)ncc(NC(=O)c2ccc(Oc3ccccc3)o2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- oxyfuran-2-carboxamide |
| 58 | 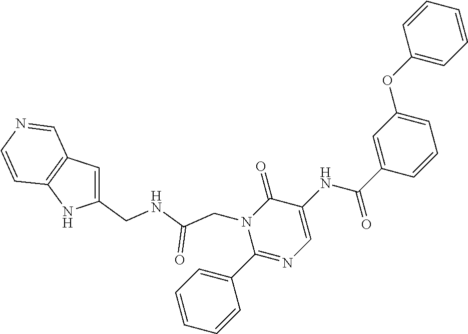 [SMILES: O=C(Cn1c(-c2ccccc2)ncc(NC(=O)c2cccc(Oc3ccccc3)c2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-3-phen- oxybenzamide |
| 59 | 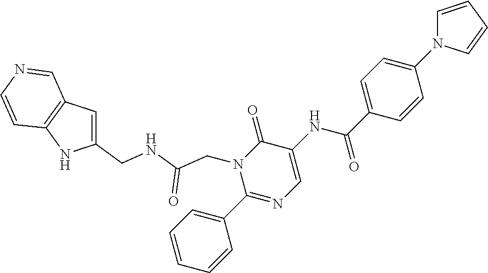 [SMILES: O=C(Cn1c(-c2ccccc2)ncc(NC(=O)c2ccc(-n3cccc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(1H- pyrrol-1-yl)benzamide |
| 60 | 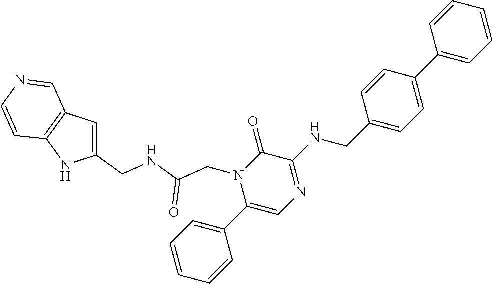 [SMILES: O=C(Cn1c(-c2ccccc2)cnc(NCc2ccc(-c3ccccc3)cc2)c1=O)NCc1cc2cnccc2[nH]1] | N-((1H-pyrrolo[3,2-c]pyridin- 2-yl)methyl)-2-(3-(([1,1′- biphenyl]-4-ylmethyl)amino)- 2-oxo-6-phenylpyrazin-1(2H)- yl)acetamide |
| 61 | 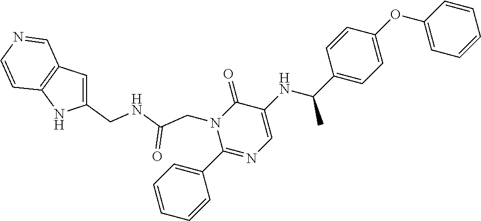 [SMILES: C[C@@H](Nc1cnc(-c2ccccc2)n(CC(=O)NCc2cc3cnccc3[nH]2)c1=O)c1ccc(Oc2ccccc2)cc1] | (R)-N-((1H-pyrrolo[3,2-c]pyr- idin-2-yl)methyl)-2-(6-oxo-5- ((1-(4-phenoxyphenyl)ethyl)- amino)-2-phenylpyrimidin- 1(6H)-yl)acetamide |
| 62 | 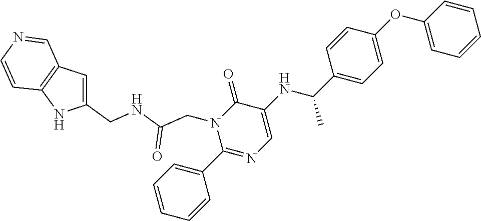 [SMILES: C[C@H](Nc1cnc(-c2ccccc2)n(CC(=O)NCc2cc3cnccc3[nH]2)c1=O)c1ccc(Oc2ccccc2)cc1] | (S)-N-((1H-pyrrolo[3,2-c]pyr- idin-2-yl)methyl)-2-(6-oxo-5- ((1-(4-phenoxyphenyl)ethyl)- amino)-2-phenylpyrimidin- 1(6H)-yl)acetamide |
| 63 | 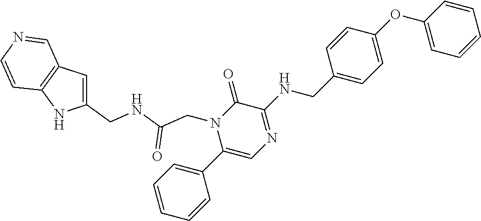 [SMILES: O=C(Cn1c(-c2ccccc2)cnc(NCc2ccc(Oc3ccccc3)cc2)c1=O)NCc1cc2cnccc2[nH]1] | N-((1H-pyrrolo[3,2-c]pyridin- 2-yl)methyl)-2-(2-oxo-3-((4- phenoxybenzyl)amino)-6-phen- ylpyrazin-1(2H)-yl)acetamide |
| 64 | 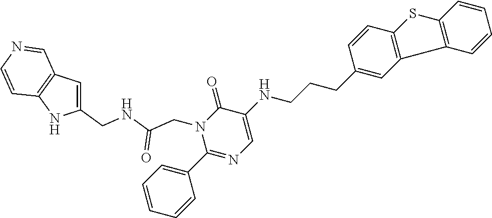 [SMILES: O=C(Cn1c(-c2ccccc2)ncc(NCCCc2ccc3sc4ccccc4c3c2)c1=O)NCc1cc2cnccc2[nH]1] | N-((1H-pyrrolo[3,2-c]pyridin- 2-yl)methyl)-2-(5-((3-(dibenzo [b,d]thiophen-2-yl)propyl)- amino)-6-oxo-2-phenylpyrimi- din-1(6H)-yl)acetamide |
| 65 | 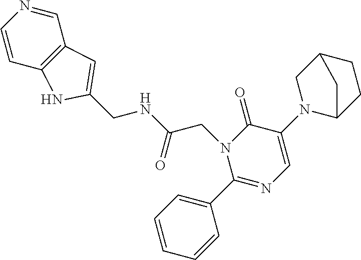 [SMILES: O=C(Cn1c(-c2ccccc2)ncc(N2CC3CCC2C3)c1=O)NCc1cc2cnccc2[nH]1] | N-((1H-pyrrolo[3,2-c]pyridin- 2-yl)methyl)-2-(5-(2-azabicyclo [2.2.1]heptan-2-yl)-6-oxo-2- phenylpyrimidin-1(6H)-yl)- acetamide |
| 66 | 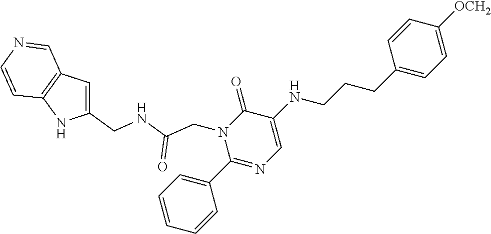 | N-((1H-pyrrolo[3,2-c]pyridin- 2-yl)methyl)-2-(5-((3-(4- (fluoromethoxy)phenyl)prop- yl)amino)-6-oxo-2-phenylpyr- imidin-1(6H)-yl)acetamide |
| 67 | 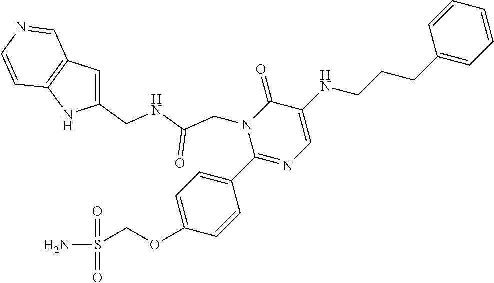 [SMILES: NS(=O)(=O)COc1ccc(-c2ncc(NCCCc3ccccc3)c(=O)n2CC(=O)NCc2cc3cnccc3[nH]2)cc1] | N-((1H-pyrrolo[3,2-c]pyridin- 2-yl)methyl)-2-(6-oxo-5-((3- phenylpropyl)amino)-2-(4- (sulfamoylmethoxy)phenyl)pyr- imidin-1(6H)-yl)acetamide |
| 68 | 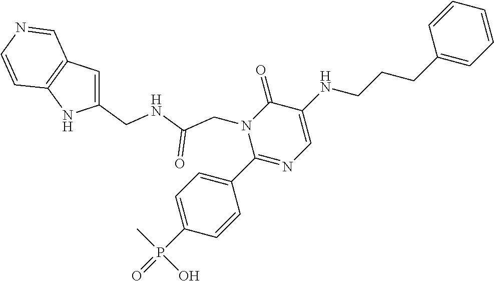 [SMILES: CP(=O)(O)c1ccc(-c2ncc(NCCCc3ccccc3)c(=O)n2CC(=O)NCc2cc3cnccc3[nH]2)cc1] | (4-(1-(2-(((1H-pyrrolo[3,2-c] pyridin-2-yl)methyl)amino)-2- oxoethyl)-6-oxo-5-((3-phenyl- propyl)amino)-1,6-dihydropyr- imidin-2-yl)phenyl)(methyl)- phosphinic acid |
| 69 | 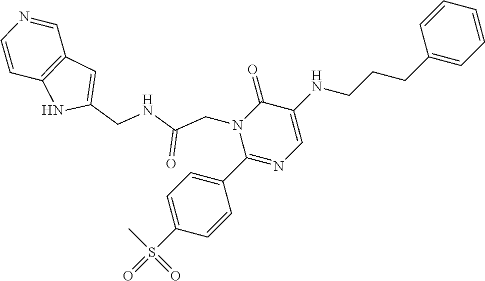 [SMILES: CS(=O)(=O)c1ccc(-c2ncc(NCCCc3ccccc3)c(=O)n2CC(=O)NCc2cc3cnccc3[nH]2)cc1] | N-((1H-pyrrolo[3,2-c]pyridin- 2-yl)methyl)-2-(2-(4-(methyl- sulfonyl)phenyl)-6-oxo-5-((3- phenylpropyl)amino)pyrimidin- 1(6H)-yl)acetamide |
| 70 | 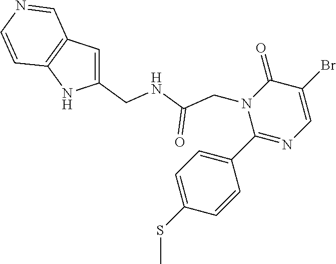 [SMILES: CSc1ccc(-c2ncc(Br)c(=O)n2CC(=O)NCc2cc3cnccc3[nH]2)cc1] | N-((1H-pyrrolo[3,2-c]pyridin- 2-yl)methyl)-2-(5-bromo-2- (methylthio)-6-oxopyrimidin- 1(6H)-yl)acetamide |
| 71 | 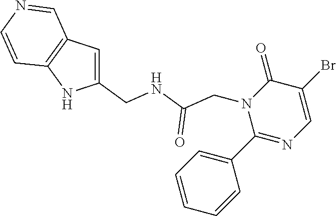 [SMILES: O=C(Cn1c(-c2ccccc2)ncc(Br)c1=O)NCc1cc2cnccc2[nH]1] | N-((1H-pyrrolo[3,2-c]pyridin- 2-yl)methyl)-2-(5-bromo-6- oxo-2-phenylpyrimidin-1(6H)- yl)acetamide |
| 72 | 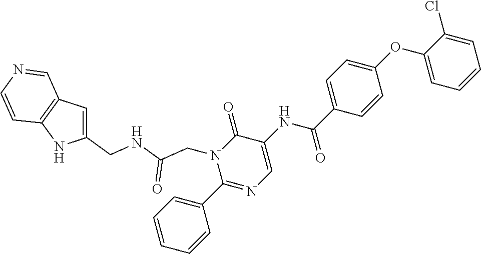 [SMILES: O=C(Cn1c(-c2ccccc2)ncc(NC(=O)c2ccc(Oc3ccccc3Cl)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(2- chlorophenoxy)benzamide |
| 73 | 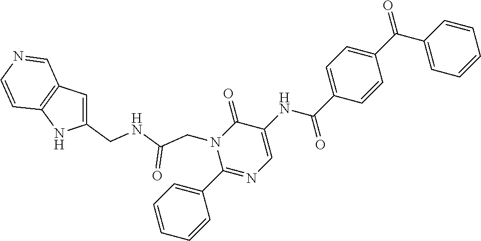 [SMILES: O=C(Cn1c(-c2ccccc2)ncc(NC(=O)c2ccc(C(=O)c3ccccc3)cc2)c1=O)NCc1cc2cnccc2[nH]1] | N-(1-(2-(((1H-pyrrolo[3,2-c] pyridin-2-yl)methyl)amino)- 2-oxoethyl)-6-oxo-2-phenyl- 1,6-dihydropyrimidin-5-yl)-4- benzoylbenzamide |
| 74 | 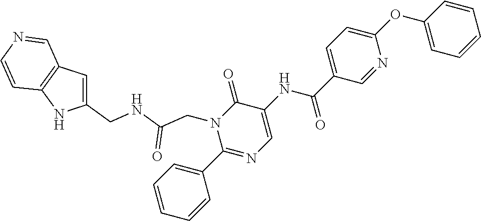 [SMILES: O=C(Cn1c(-c2ccccc2)ncc(NC(=O)c2ccc(Oc3ccccc3)nc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-6- phenoxynicotinamide |
| 75 | 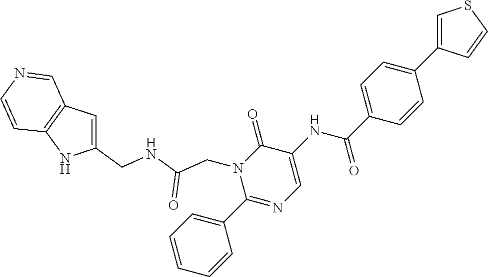 [SMILES: O=C(Cn1c(-c2ccccc2)ncc(NC(=O)c2ccc(-c3ccsc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4- (thiophen-3-yl)benzamide |
| 76 | 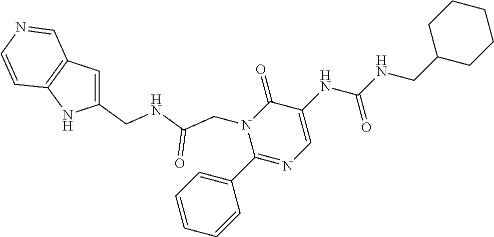 [SMILES: O=C(Cn1c(-c2ccccc2)ncc(NC(=O)NCC2CCCCC2)c1=O)NCc1cc2cnccc2[nH]1] | N-((1H-pyrrolo[3,2-c]pyridin- 2-yl)methyl)-2-(5-(3-(cyclo- hexylmethyl)ureido)-6-oxo-2- phenylpyrimidin-1(6H)-yl)- acetamide |
| 77 | 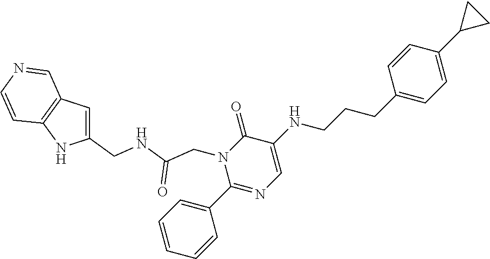 [SMILES: O=C(Cn1c(-c2ccccc2)ncc(NCCCc2ccc(C3CC3)cc2)c1=O)NCc1cc2cnccc2[nH]1] | N-((1H-pyrrolo[3,2-c]pyridin- 2-yl)methyl)-2-(5-((3-(4-cyclo- propylphenyl)propyl)amino)-6- oxo-2-phenylpyrimidin-1(6H)- yl)acetamide |
| 78 | 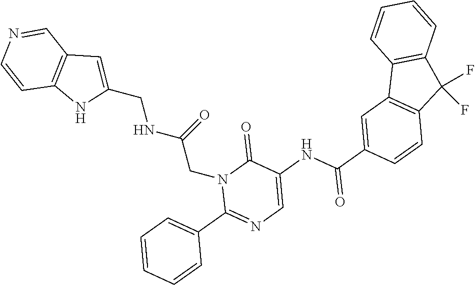 [SMILES: O=C(Cn1c(-c2ccccc2)ncc(NC(=O)c2ccc3c(c2)-c2ccccc2C3(F)F)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-9,9- difluoro-9H-fluorene-3-carbox- amide |
| 79 | 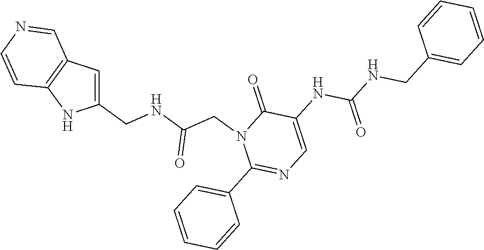 [SMILES: O=C(Cn1c(-c2ccccc2)ncc(NC(=O)NCc2ccccc2)c1=O)NCc1cc2cnccc2[nH]1] | N-((1H-pyrrolo[3,2-c]pyridin- 2-yl)methyl)-2-(5-(3-benzyl- ureido)-6-oxo-2-phenylpyr- imidin-1(6H)-yl)acetamide |
| 80 | 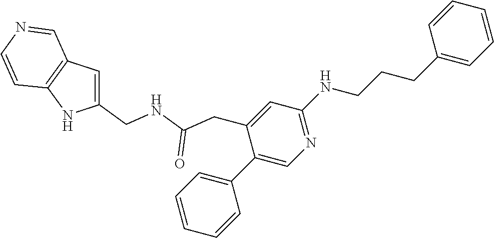 [SMILES: O=C(Cc1cc(NCCCc2ccccc2)ncc1-c1ccccc1)NCc1cc2cnccc2[nH]1] | N-((1H-pyrrolo[3,2-c]pyridin- 2-yl)methyl)-2-(5-phenyl-2-((3- phenylpropyl)amino)pyridin-4- yl)acetamide |
| 81 | 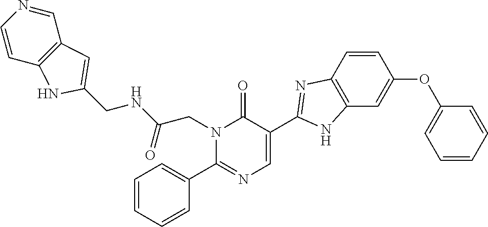 [SMILES: O=C(Cn1c(-c2ccccc2)ncc(-c2nc3ccc(Oc4ccccc4)cc3[nH]2)c1=O)NCc1cc2cnccc2[nH]1] | N-((1H-pyrrolo[3,2-c]pyridin- 2-yl)methyl)-2-(6-oxo-5-(6- phenoxy-1H-benzo[d]imidazol- 2-yl)-2-phenylpyrimidin-1(6H)- yl)acetamide |
| 82 | 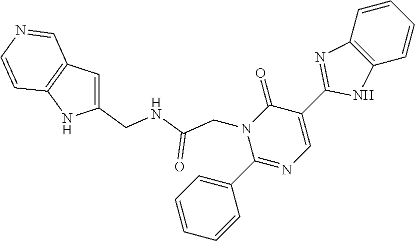 [SMILES: O=C(Cn1c(-c2ccccc2)ncc(-c2nc3ccccc3[nH]2)c1=O)NCc1cc2cnccc2[nH]1] | 2-(5-(1H-benzo[d]imidazol-2- yl)-6-oxo-2-phenylpyrimidin- 1(6H)-yl)-N-((1H-pyrrolo[3,2- c]pyridin-2-yl)methyl)acet- amide |
| 83 | 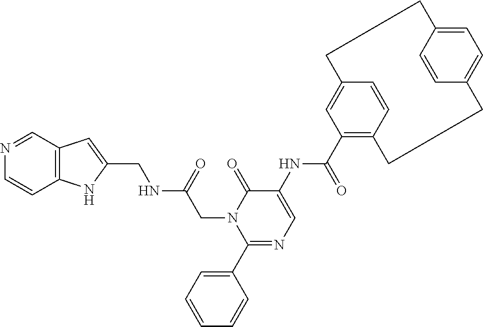 [SMILES: O=C(Cn1c(-c2ccccc2)ncc(NC(=O)c2cc3ccc2CCc2ccc(cc2)CC3)c1=O)NCc1cc2cnccc2[nH]1] | N-(1-(2-(((1H-pyrrolo[3,2-c] pyridin-2-yl)methyl)amino)- 2-oxoethyl)-6-oxo-2-phenyl- 1,6-dihydropyrimidin-5-yl)- 1,4(1,4)-dibenzenacyclohexa- phane-1(2)-carboxamide |
| 84 | 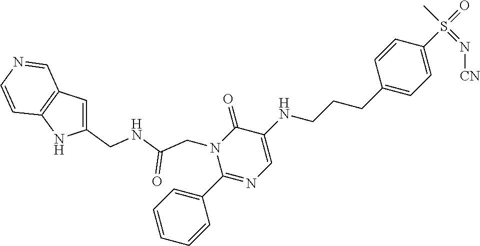 [SMILES: CS(=O)(=NC#N)c1ccc(CCCNc2cnc(-c3ccccc3)n(CC(=O)NCc3cc4cnccc4[nH]3)c2=O)cc1] | N-((1H-pyrrolo[3,2-c]pyridin- 2-yl)methyl)-2-(5-((3-(4-(N- cyano-S-methylsulfonimido- yl)phenyl)propyl)amino)-6- oxo-2-phenylpyrimidin-1(6H)- yl)acetamide |
| 85 | 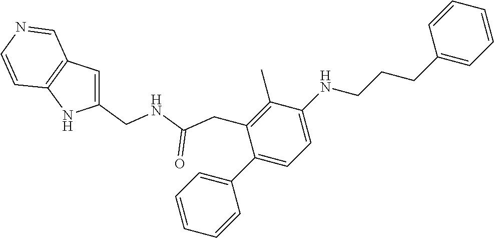 [SMILES: Cc1c(NCCCc2ccccc2)ccc(-c2ccccc2)c1CC(=O)NCc1cc2cnccc2[nH]1] | N-((1H-pyrrolo[3,2-c]pyridin- 2-yl)methyl)-2-(3-methyl-4- ((3-phenylpropyl)amino)-[1,1′- biphenyl]-2-yl)acetamide |
| 86 | 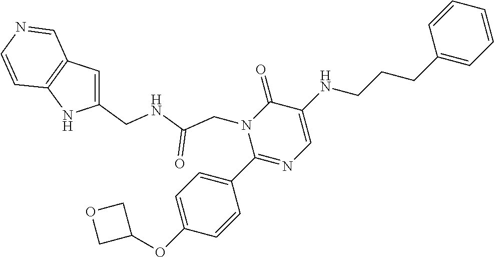 [SMILES: O=C(Cn1c(-c2ccc(OC3COC3)cc2)ncc(NCCCc2ccccc2)c1=O)NCc1cc2cnccc2[nH]1] | N-((1H-pyrrolo[3,2-c]pyridin- 2-yl)methyl)-2-(2-(4-(oxetan- 3-yloxy)phenyl)-6-oxo-5-((3- phenylpropyl)amino)pyrimidin- 1(6H)-yl)acetamide |
| 87 | 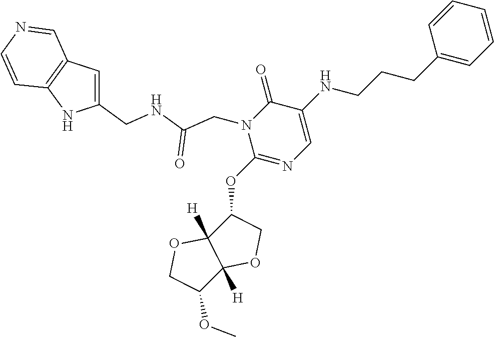 [SMILES: CO[C@@H]1CO[C@H]2[C@@H]1OC[C@H]2Oc1ncc(NCCCc2ccccc2)c(=O)n1CC(=O)NCc1cc2cnccc2[nH]1] | N-((1H-pyrrolo[3,2-c]pyridin- 2-yl)methyl)-2-(2-(((3R,3aR, 6R,6aR)-6-methoxyhexahydro- furo[3,2-b]furan-3-yl)oxy)-6- oxo-5-((3-phenylpropyl)- amino)pyrimidin-1(6H)-yl)- acetamide |
| 88 | 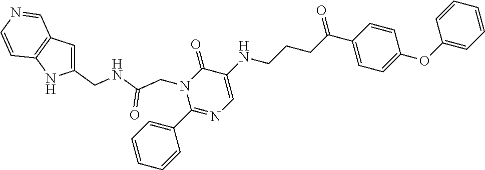 [SMILES: O=C(Cn1c(-c2ccccc2)ncc(NCCCC(=O)c2ccc(Oc3ccccc3)cc2)c1=O)NCc1cc2cnccc2[nH]1] | N-((1H-pyrrolo[3,2-c]pyridin- 2-yl)methyl)-2-(6-oxo-5-((4- oxo-4-(4-phenoxyphenyl)but- yl)amino)-2-phenylpyrimidin- 1(6H)-yl)acetamide |
| 89 | 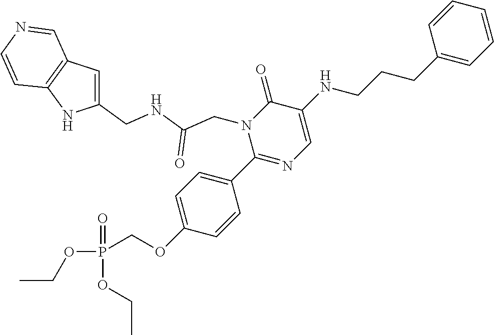 [SMILES: CCOP(=O)(COc1ccc(-c2ncc(NCCCc3ccccc3)c(=O)n2CC(=O)NCc2cc3cnccc3[nH]2)cc1)OCC] | diethyl ((4-(1-(2-(((1H-pyrrolo [3,2-c]pyridin-2-yl)methyl)- amino)-2-oxoethyl)-6-oxo-5- ((3-phenylpropyl)amino)-1,6- dihydropyrimidin-2-yl)phen- oxy)methyl)phosphonate |
| 90 | 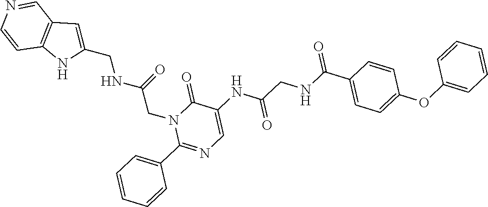 [SMILES: O=C(Cn1c(-c2ccccc2)ncc(NC(=O)CNC(=O)c2ccc(Oc3ccccc3)cc2)c1=O)NCc1cc2cnccc2[nH]1] | N-(2-((1-(2-(((1H-pyrrolo[3,2- c]pyridin-2-yl)methyl)amino)- 2-oxoethyl)-6-oxo-2-phenyl- 1,6-dihydropyrimidin-5-yl)- amino)-2-oxoethyl)-4-phenoxy- benzamide |
| 91 | 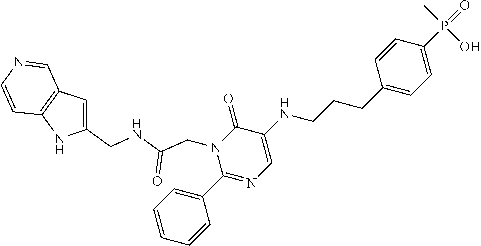 [SMILES: CP(=O)(O)c1ccc(CCCNc2cnc(-c3ccccc3)n(CC(=O)NCc3cc4cnccc4[nH]3)c2=O)cc1] | (4-(3-((1-(2-(((1H-pyrrolo[3,2- c]pyridin-2-yl)methyl)amino)- 2-oxoethyl)-6-oxo-2-phenyl- 1,6-dihydropyrimidin-5-yl)- amino)propyl)phenyl)(methyl)- phosphinic acid |
| 92 | 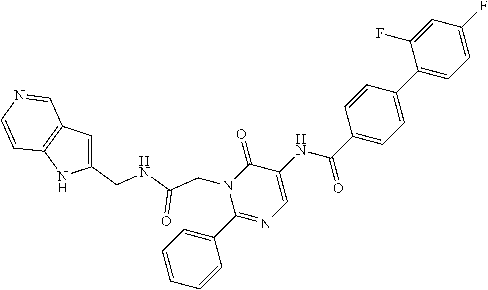 [SMILES: O=C(Cn1c(-c2ccccc2)ncc(NC(=O)c2ccc(-c3ccc(F)cc3F)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2′,4′- difluoro-[1,1′-biphenyl]-4- carboxamide |
| 93 | 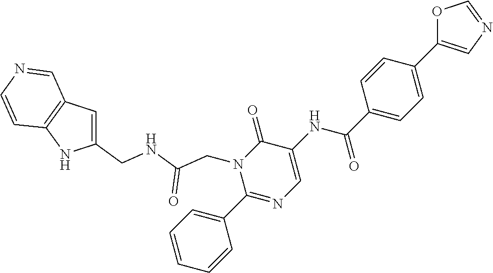 [SMILES: O=C(Cn1c(-c2ccccc2)ncc(NC(=O)c2ccc(-c3cnco3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4- (oxazol-5-yl)benzamide |
| 94 | 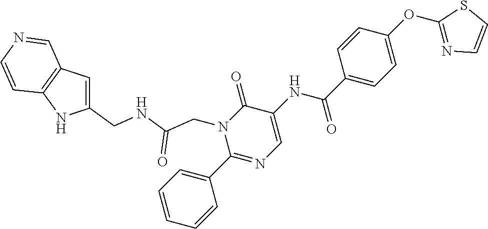 [SMILES: O=C(Cn1c(-c2ccccc2)ncc(NC(=O)c2ccc(Oc3nccs3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4- (thiazol-2-yloxy)benzamide |
| 95 | 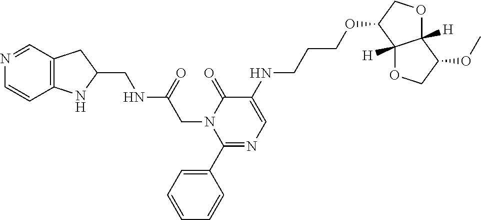 [SMILES: CO[C@@H]1CO[C@H]2[C@@H]1OC[C@H]2OCCCNc1cnc(-c2ccccc2)n(CC(=O)NCC2Cc3cnccc3N2)c1=O] | N-((1H-pyrrolo[3,2-c]pyridin- 2-yl)methyl)-2-(5-((3-(((3R, 3aR,6R,6aR)-6-methoxyhexa- hydrofuro[3,2-b]furan-3-yl)- oxy)propyl)amino)-6-oxo-2- phenylpyrimidin-1(6H)-yl)- acetamide |
| 96 | 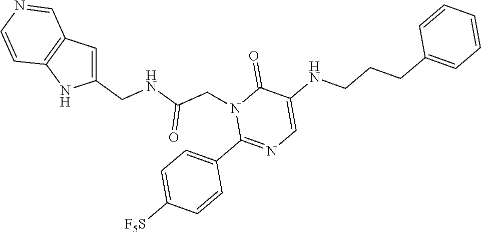 | N-((1H-pyrrolo[3,2-c]pyridin- 2-yl)methyl)-2-(6-oxo-2-(4- (pentafluoro-lambda6-sulfane- yl)phenyl)-5-((3-phenylprop- yl)amino)pyrimidin-1(6H)- yl)acetamide |
| 97 | 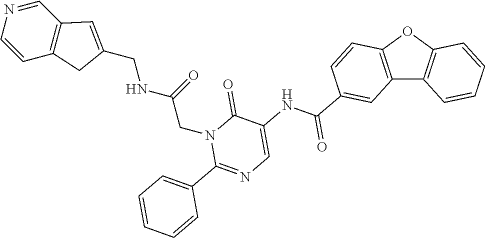 [SMILES: O=C(Cn1c(-c2ccccc2)ncc(NC(=O)c2ccc3oc4ccccc4c3c2)c1=O)NCC1=Cc2cnccc2C1] | N-(1-(2-(((1H-pyrrolo[3,2-c] pyridin-2-yl)methyl)amino)-2- oxoethyl)-6-oxo-2-phenyl-1,6- dihydropyrimidin-5-yl)dibenzo [b,d]furan-2-carboxamide |
| 98 | 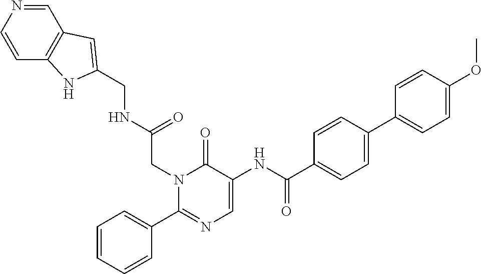 [SMILES: COc1ccc(-c2ccc(C(=O)Nc3cnc(-c4ccccc4)n(CC(=O)NCc4cc5cnccc5[nH]4)c3=O)cc2)cc1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4′- methoxy-[1,1′-biphenyl]-4- carboxamide |
| 99 | 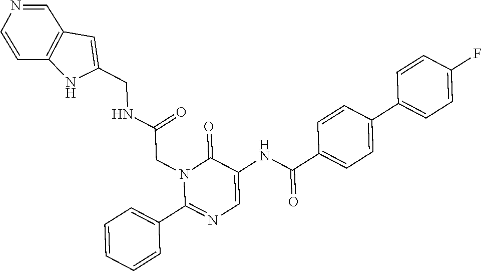 [SMILES: O=C(Cn1c(-c2ccccc2)ncc(NC(=O)c2ccc(-c3ccc(F)cc3)cc2)c1=O)NCc1cc2cnccc2[nH]1] | N-(1-(2-(((1H-pyrrolo[3,2-c] pyridin-2-yl)methyl)amino)- 2-oxoethyl)-6-oxo-2-phenyl- 1,6-dihydropyrimidin-5-yl)- 4′-fluoro-[1,1′-biphenyl]-4- carboxamide |
| 100 | 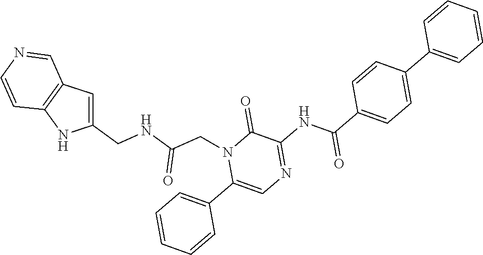 [SMILES: O=C(Cn1c(-c2ccccc2)cnc(NC(=O)c2ccc(-c3ccccc3)cc2)c1=O)NCc1cc2cnccc2[nH]1] | N-(4-(2-(((1H-pyrrolo[3,2-c] pyridin-2-yl)methyl)amino)-2- oxoethyl)-3-oxo-5-phenyl-3,4- dihydropyrazin-2-yl)-[1,1′- biphenyl]-4-carboxamide |
| 101 | 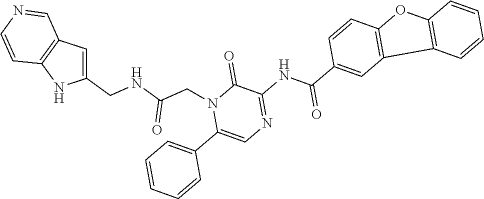 [SMILES: O=C(Cn1c(-c2ccccc2)cnc(NC(=O)c2ccc3oc4ccccc4c3c2)c1=O)NCc1cc2cnccc2[nH]1] | N-(4-(2-(((1H-pyrrolo[3,2-c] pyridin-2-yl)methyl)amino)-2- oxoethyl)-3-oxo-5-phenyl-3,4- dihydropyrazin-2-yl)dibenzo [b,d]furan-2-carboxamide |
| 102 | 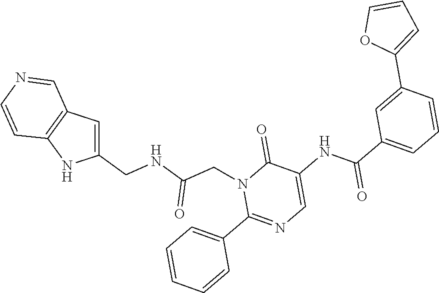 [SMILES: O=C(Cn1c(-c2ccccc2)ncc(NC(=O)c2cccc(-c3ccco3)c2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-3- (furan-2-yl)benzamide |
| 103 | 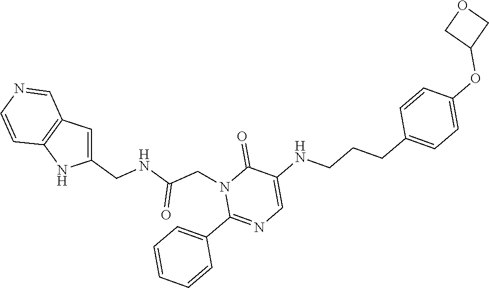 [SMILES: O=C(Cn1c(-c2ccccc2)ncc(NCCCc2ccc(OC3COC3)cc2)c1=O)NCc1cc2cnccc2[nH]1] | N-((1H-pyrrolo[3,2-c]pyridin- 2-yl)methyl)-2-(5-((3-(4-(oxe- tan-3-yloxy)phenyl)propyl)- amino)-6-oxo-2-phenylpyrimi- din-1(6H)-yl)acetamide |
| 104 | 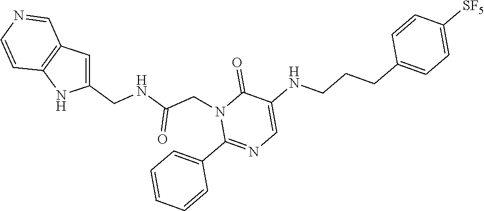 | N-((1H-pyrrolo[3,2-c]pyridin- 2-yl)methyl)-2-(6-oxo-5-((3- (4-(pentafluoro-lambda6- sulfaneyl)phenyl)propyl)- amino)-2-phenylpyrimidin- 1(6H)-yl)acetamide |
| 105 | 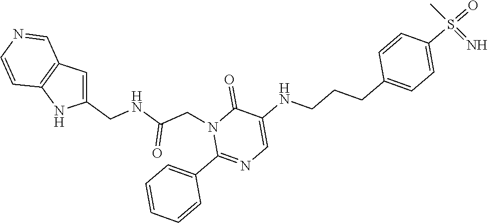 [SMILES: CS(=N)(=O)c1ccc(CCCNc2cnc(-c3ccccc3)n(CC(=O)NCc3cc4cnccc4[nH]3)c2=O)cc1] | N-((1H-pyrrolo[3,2-c]pyridin- 2-yl)methyl)-2-(5-((3-(4-(S- methylsulfonimidoyl)phenyl)- propyl)amino)-6-oxo-2-phen- ylpyrimidin-1(6H)-yl)acet- amide |
| 106 | 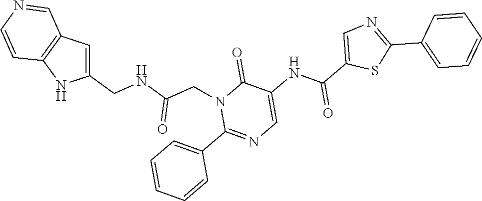 [SMILES: O=C(Cn1c(-c2ccccc2)ncc(NC(=O)c2cnc(-c3ccccc3)s2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2- phenylthiazole-5-carboxamide |
| 107 | 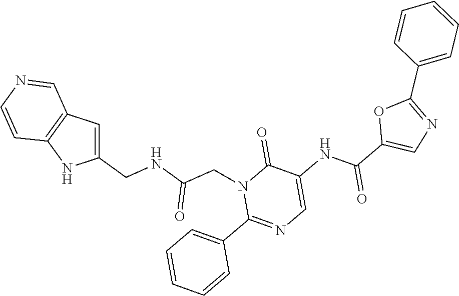 [SMILES: O=C(Cn1c(-c2ccccc2)ncc(NC(=O)c2cnc(-c3ccccc3)o2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2- phenyloxazole-5-carboxamide |
| 108 | 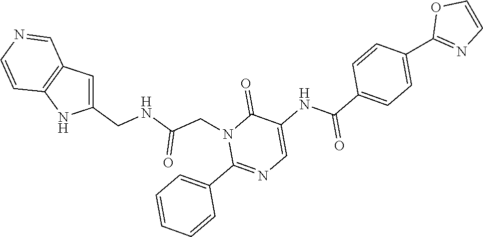 [SMILES: O=C(Cn1c(-c2ccccc2)ncc(NC(=O)c2ccc(-c3ncco3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4- (oxazol-2-yl)benzamide |
| 109 | 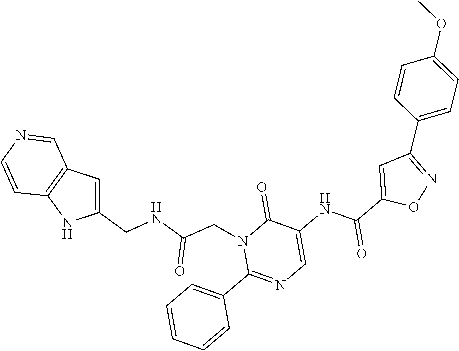 [SMILES: COc1ccc(-c2cc(C(=O)Nc3cnc(-c4ccccc4)n(CC(=O)NCc4cc5cnccc5[nH]4)c3=O)on2)cc1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-3-(4- methoxyphenyl)isoxazole-5- carboxamide |
| 110 | 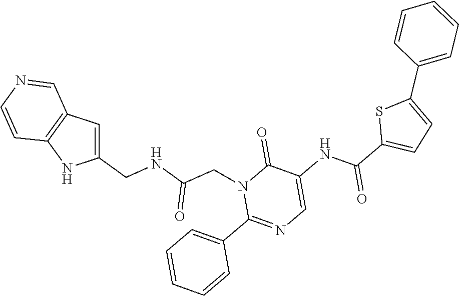 [SMILES: O=C(Cn1c(-c2ccccc2)ncc(NC(=O)c2ccc(-c3ccccc3)s2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5- phenylthiophene-2-carbox- amide |
| 111 | 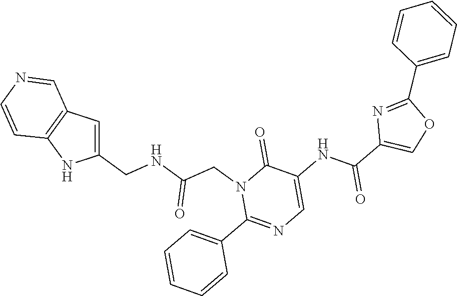 [SMILES: O=C(Cn1c(-c2ccccc2)ncc(NC(=O)c2coc(-c3ccccc3)n2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2- phenyloxazole-4-carboxamide |
| 112 | 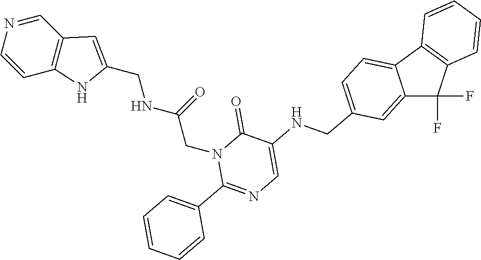 [SMILES: O=C(Cn1c(-c2ccccc2)ncc(NCc2ccc3c(c2)C(F)(F)c2ccccc2-3)c1=O)NCc1cc2cnccc2[nH]1] | N-((1H-pyrrolo[3,2-c]pyridin- 2-yl)methyl)-2-(5-(((9,9- difluoro-9H-fluoren-2-yl)meth- yl)amino)-6-oxo-2-phenyl- pyrimidin-1(6H)-yl)acetamide |
| 113 | 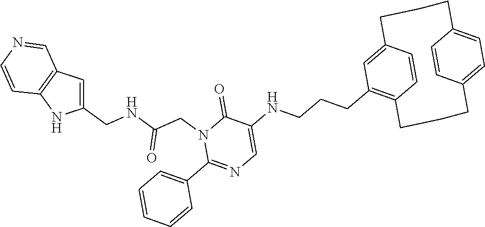 [SMILES: O=C(Cn1c(-c2ccccc2)ncc(NCCCc2cc3ccc2CCCc2ccc(cc2)CCC3)c1=O)NCc1cc2cnccc2[nH]1] | 2-(5-((3-(1,4(1,4)-dibenzena- cyclohexaphane-1(2)-yl)prop- yl)amino)-6-oxo-2-phenyl- pyrimidin-1(6H)-yl)-N-((1H- pyrrolo[3,2-c]pyridin-2-yl)- methyl)acetamide |
| 114 | 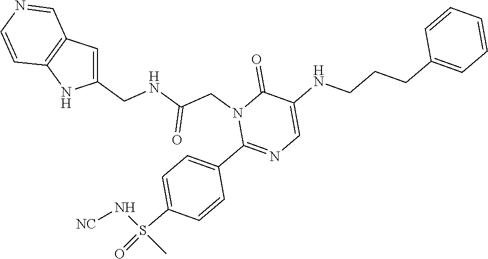 | N-((1H-pyrrolo[3,2-c]pyridin- 2-yl)methyl)-2-(2-(4-(N-cyano- S-methylsulfonimidoyl)phen- yl)-6-oxo-5-((3-phenylpropyl)- amino)pyrimidin-1(6H)-yl)- acetamide |
| 115 | 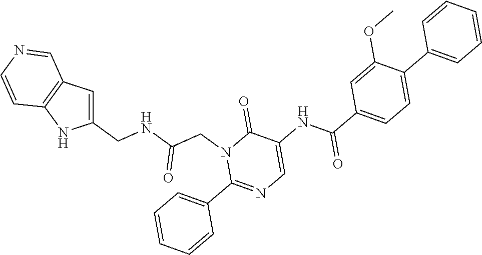 [SMILES: COc1cc(C(=O)Nc2cnc(-c3ccccc3)n(CC(=O)NCc3cc4cnccc4[nH]3)c2=O)ccc1-c1ccccc1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2- methoxy-[1,1′-biphenyl]-4- carboxamide |
| 116 | 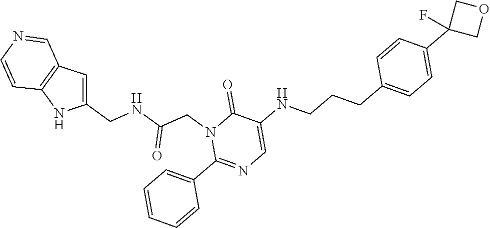 [SMILES: O=C(Cn1c(-c2ccccc2)ncc(NCCCc2ccc(C3(F)COC3)cc2)c1=O)NCc1cc2cnccc2[nH]1] | N-((1H-pyrrolo[3,2-c]pyridin- 2-yl)methyl)-2-(5-((3-(4-(3- fluorooxetan-3-yl)phenyl)- propyl)amino)-6-oxo-2-phen- ylpyrimidin-1(6H)-yl)acet- amide |
| 117 | 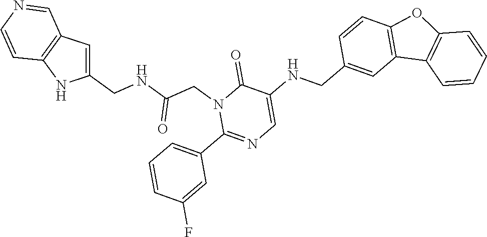 [SMILES: O=C(Cn1c(-c2cccc(F)c2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(3-fluorophenyl)-6-oxopyr- imidin-1(6H)-yl)acetamide |
| 118 | 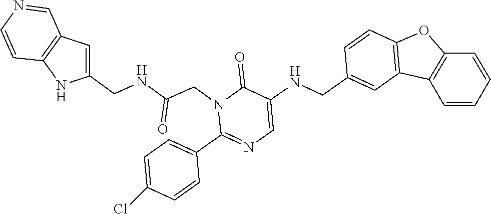 [SMILES: O=C(Cn1c(-c2ccc(Cl)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(2-(4-chloro- phenyl)-5-((dibenzo[b,d]furan- 2-ylmethyl)amino)-6-oxopyr- imidin-1(6H)-yl)acetamide |
| 119 | 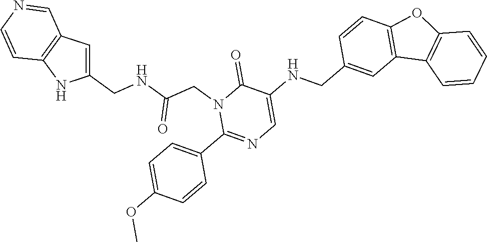 [SMILES: COc1ccc(-c2ncc(NCc3ccc4oc5ccccc5c4c3)c(=O)n2CC(=O)NCc2cc3cnccc3[nH]2)cc1] | N-((1H-pyrrolo[3,2-c]pyridin- 2-yl)methyl)-2-(5-((dibenzo [b,d]furan-2-ylmethyl)amino)- 2-(4-methoxyphenyl)-6-oxo- pyrimidin-1(6H)-yl)acetamide |
| 120 | 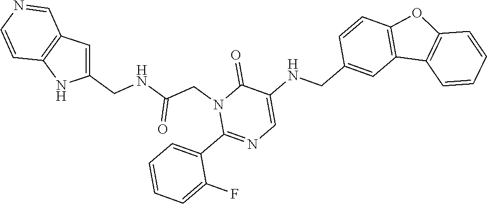 [SMILES: O=C(Cn1c(-c2ccccc2F)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(2-fluorophenyl)-6-oxopyr- imidin-1(6H)-yl)acetamide |
| 121 | 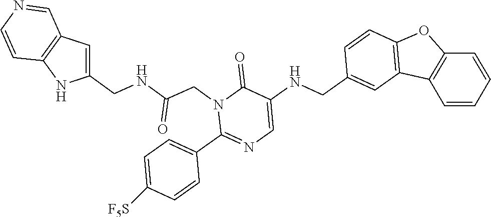 | N-((1H-pyrrolo[3,2-c]pyridin- 2-yl)methyl)-2-(5-((dibenzo [b,d]furan-2-ylmethyl)amino)- 6-oxo-2-(4-(pentafluoro-l6- sulfaneyl)phenyl)pyrimidin- 1(6H)-yl)acetamide |
| 122 | 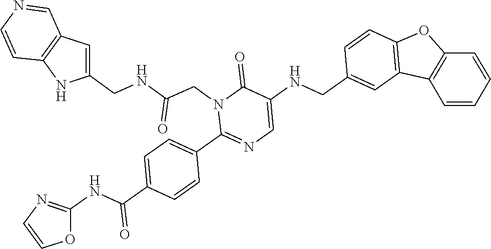 [SMILES: O=C(Cn1c(-c2ccc(C(=O)Nc3ncco3)cc2)ncc(NCc2ccc3oc4ccccc4c3c2)c1=O)NCc1cc2cnccc2[nH]1] | 4-(1-(2-(((1H-pyrrolo[3,2-c] pyridin-2-yl)methyl)amino)- 2-oxoethyl)-5-((dibenzo[b,d] furan-2-ylmethyl)amino)-6- oxo-1,6-dihydropyrimidin-2- yl)-N-(oxazol-2-yl)benzamide |
| 123 | 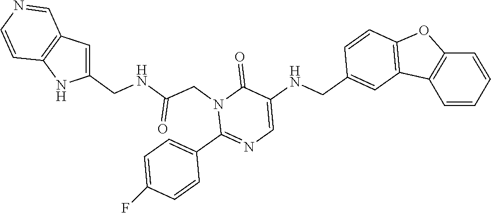 [SMILES: O=C(Cn1c(-c2ccc(F)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(4-fluorophenyl)-6-oxopyr- imidin-1(6H)-yl)acetamide |
| 124 | 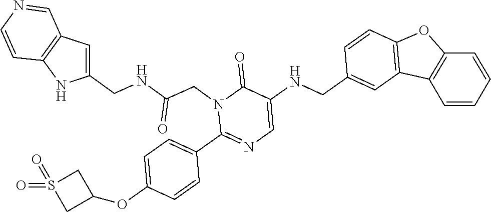 [SMILES: O=C(Cn1c(-c2ccc(OC3CS(=O)(=O)C3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(2-(4-((1,1- dioxidothietan-3-yl)oxy)phen- yl)-6-oxo-5-((3-phenylprop- yl)amino)pyrimidin-1(6H)- yl)acetamide |
| 125 | 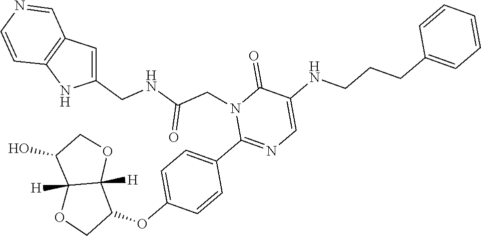 | N-((1H-pyrrolo[3,2-c]pyridin- 2-yl)methyl)-2-(2-(4-(((3R, 3aR,6R,6aR)-6-hydroxyhexa- hydrofuro[3,2-b]furan-3-yl)- oxy)phenyl)-6-oxo-5-((3-phen- ylpropyl)amino)pyrimidin- 1(6H)-yl)acetamide |
| 126 | 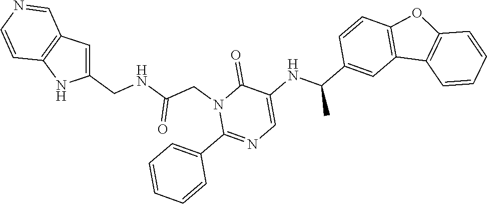 [SMILES: C[C@@H](Nc1cnc(-c2ccccc2)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(5-((1- (dibenzo[b,d]furan-2-yl)ethyl)- amino)-6-oxo-2-phenylpyrimi- din-1(6H)-yl)acetamide |
| 127 | 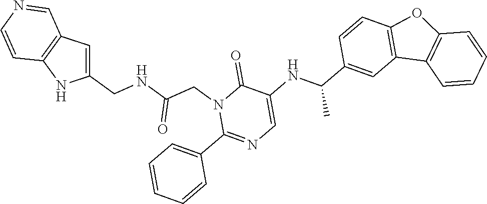 [SMILES: C[C@H](Nc1cnc(-c2ccccc2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-6-oxo-2-phenylpyrimi- din-1(6H)-yl)acetamide |
| 128 | 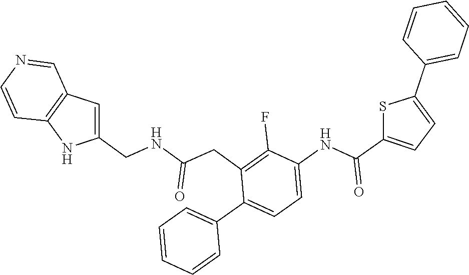 [SMILES: O=C(Cc1c(-c2ccccc2)ccc(NC(=O)c2ccc(-c3ccccc3)s2)c1F)NCc1cc2cnccc2[nH]1] | N-(2-(2-(((1H-pyrrolo[3,2-c] pyridin-2-yl)methyl)amino)- 2-oxoethyl)-3-fluoro-[1,1′- biphen-yl]-4-yl)-5-phenylthio- phene-2-carboxamide |
| 129 | 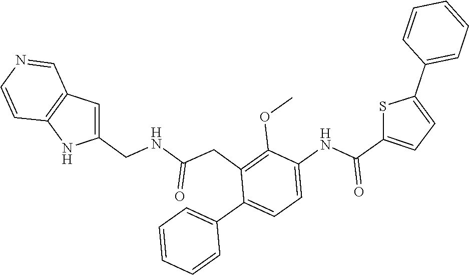 [SMILES: COc1c(NC(=O)c2ccc(-c3ccccc3)s2)ccc(-c2ccccc2)c1CC(=O)NCc1cc2cnccc2[nH]1] | N-(2-(2-(((1H-pyrrolo[3,2-c] pyridin-2-yl)methyl)amino)-2- oxoethyl)-3-methoxy-[1,1′- biphenyl]-4-yl)-5-phenylthio- phene-2-carboxamide |
| 130 | 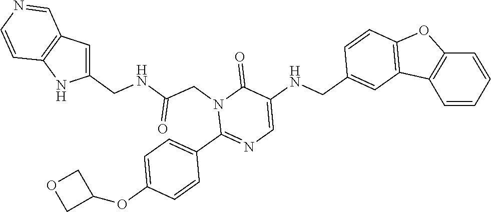 [SMILES: O=C(Cn1c(-c2ccc(OC3COC3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(4-(oxetan-3-yloxy)phenyl)- 6-oxopyrimidin-1(6H)-yl)acet- amide |
| 131 | 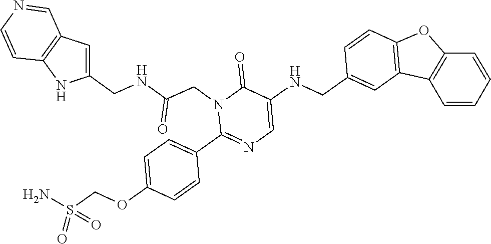 [SMILES: NS(=O)(=O)COc1ccc(-c2ncc(NCc3ccc4oc5ccccc5c4c3)c(=O)n2CC(=O)NCc2cc3cnccc3[nH]2)cc1] | N-((1H-pyrrolo[3,2-c]pyridin- 2-yl)methyl)-2-(5-((dibenzo [b,d]furan-2-ylmethyl)amino)- 6-oxo-2-(4-(sulfamoylmeth- oxy)phenyl)pyrimidin-1(6H)- yl)acetamide |
| 132 | 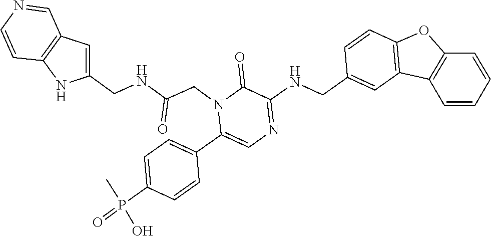 [SMILES: CP(=O)(O)c1ccc(-c2cnc(NCc3ccc4oc5ccccc5c4c3)c(=O)n2CC(=O)NCc2cc3cnccc3[nH]2)cc1] | (4-(1-(2-(((1H-pyrrolo[3,2-c] pyridin-2-yl)methyl)amino)-2- oxoethyl)-5-((dibenzo[b,d] furan-2-ylmethyl)amino)-6- oxo-1,6-dihydropyrimidin-2- yl)phenyl)(methyl)phosphinic acid |
| 133 | 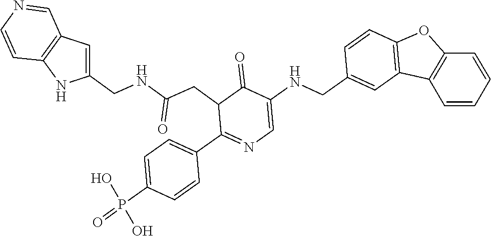 [SMILES: O=C(CC1C(=O)C(NCc2ccc3oc4ccccc4c3c2)=CN=C1c1ccc(P(=O)(O)O)cc1)NCc1cc2cnccc2[nH]1] | (4-(1-(2-(((1H-pyrrolo[3,2-c] pyridin-2-yl)methyl)amino)-2- oxoethyl)-5-((dibenzo[b,d] furan-2-ylmethyl)amino)-6- oxo-1,6-dihydropyrimidin-2- yl)phenyl)phosphonic acid |
| 134 | 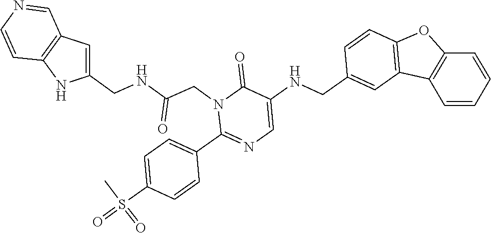 [SMILES: CS(=O)(=O)c1ccc(-c2ncc(NCc3ccc4oc5ccccc5c4c3)c(=O)n2CC(=O)NCc2cc3cnccc3[nH]2)cc1] | N-((1H-pyrrolo[3,2-c]pyridin- 2-yl)methyl)-2-(5-((dibenzo [b,d]furan-2-ylmethyl)amino)- 2-(4-(methylsulfonyl)phenyl)- 6-oxopyrimidin-1(6H)-yl)- acetamide |
| 135 | 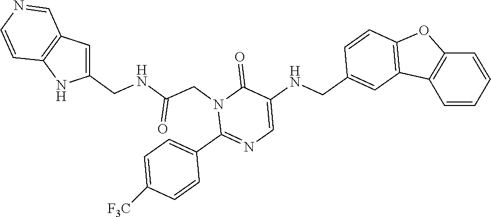 [SMILES: O=C(Cn1c(-c2ccc(C(F)(F)F)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(4-(trifluoromethyl)- phenyl)pyrimidin-1(6H)-yl)- acetamide |
| 136 | 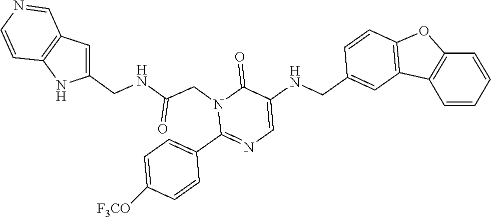 [SMILES: O=C(Cn1c(-c2ccc(OC(F)(F)F)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(4-(trifluoromethoxy)- phenyl)pyrimidin-1(6H)-yl)- acetamide |
| 137 | 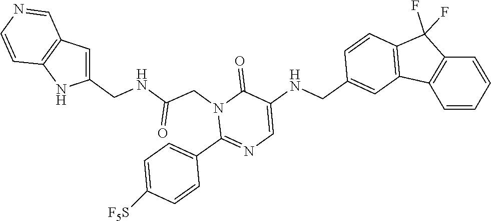 | N-((1H-pyrrolo[3,2-c]pyridin- 2-yl)methyl)-2-(5-(((9,9- difluoro-9H-fluoren-3-yl)meth- yl)amino)-6-oxo-2-(4-(penta- fluoro-l6-sulfaneyl)phenyl)- pyrimidin-1(6H)-yl)acetamide |
| 138 | 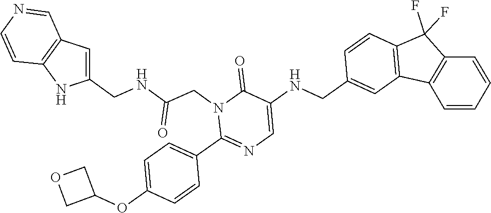 [SMILES: O=C(Cn1c(-c2ccc(OC3COC3)cc2)ncc(NCc2ccc3c(c2)-c2ccccc2C3(F)F)c1=O)NCc1cc2cnccc2[nH]1] | N-((1H-pyrrolo[3,2-c]pyridin- 2-yl)methyl)-2-(5-(((9,9- difluoro-9H-fluoren-3-yl)meth- yl)amino)-2-(4-(oxetan-3-yl- oxy)phenyl)-6-oxopyrimidin- 1(6H)-yl)acetamide |
| 139 | 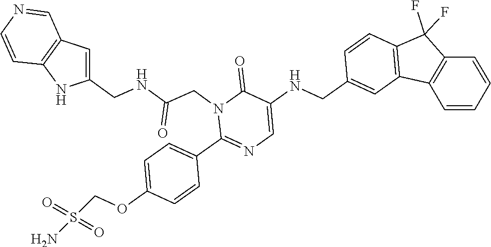 [SMILES: NS(=O)(=O)COc1ccc(-c2ncc(NCc3ccc4c(c3)-c3ccccc3C4(F)F)c(=O)n2CC(=O)NCc2cc3cnccc3[nH]2)cc1] | N-((1H-pyrrolo[3,2-c]pyridin- 2-yl)methyl)-2-(5-(((9,9- difluoro-9H-fluoren-3-yl)- methyl)amino)-6-oxo-2-(4- (sulfamoylmethoxy)phenyl)- pyrimidin-1(6H)-yl)acetamide |
| 140 | 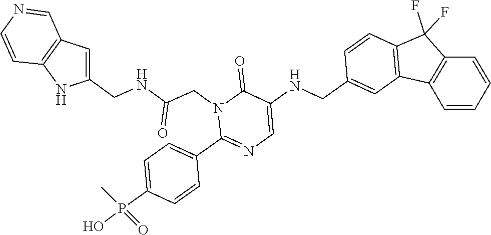 [SMILES: CP(=O)(O)c1ccc(-c2ncc(NCc3ccc4c(c3)-c3ccccc3C4(F)F)c(=O)n2CC(=O)NCc2cc3cnccc3[nH]2)cc1] | (4-(1-(2-(((1H-pyrrolo[3,2-c] pyridin-2-yl)methyl)amino)- 2-oxoethyl)-5-(((9,9-difluoro- 9H-fluoren-3-yl)methyl)- amino)-6-oxo-1,6-dihydropyr- imidin-2-yl)phenyl)(methyl)- phosphinic acid |
| 141 | 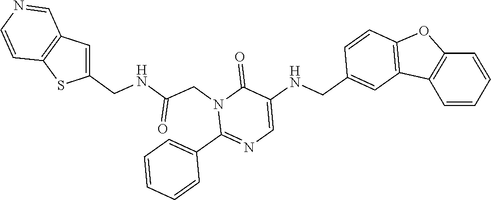 [SMILES: O=C(Cn1c(-c2ccccc2)ncc(NCc2ccc3oc4ccccc4c3c2)c1=O)NCc1cc2cnccc2s1] | 2-(5-((dibenzo[b,d]furan-2-yl- methyl)amino)-6-oxo-2-phen- ylpyrimidin-1(6H)-yl)-N- (thieno[3,2-c]pyridin-2-ylmeth- yl)acetamide |
| 142 | 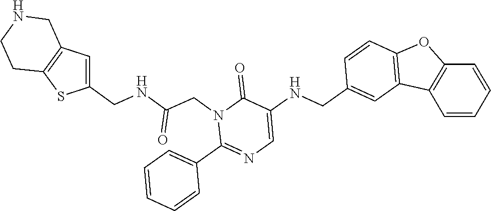 [SMILES: O=C(Cn1c(-c2ccccc2)ncc(NCc2ccc3oc4ccccc4c3c2)c1=O)NCc1cc2c(s1)CCNC2] | 2-(5-((dibenzo[b,d]furan-2-yl- methyl)amino)-6-oxo-2-phen- ylpyrimidin-1(6H)-yl)-N- ((4,5,6,7-tetrahydrothieno[3,2- c]pyridin-2-yl)methyl)acet- amide |
| 143 | 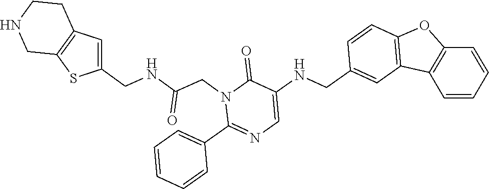 [SMILES: O=C(Cn1c(-c2ccccc2)ncc(NCc2ccc3oc4ccccc4c3c2)c1=O)NCc1cc2c(s1)CNCC2] | 2-(5-((dibenzo[b,d]furan-2-yl- methyl)amino)-6-oxo-2-phen- ylpyrimidin-1(6H)-yl)-N- ((4,5,6,7-tetrahydrothieno [2,3-c]pyridin-2-yl)methyl)- acetamide |
| 144 | 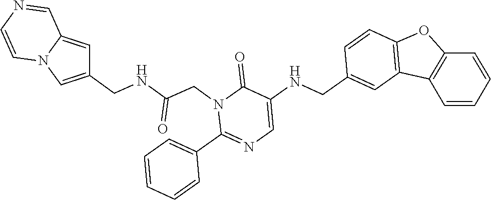 [SMILES: O=C(Cn1c(-c2ccccc2)ncc(NCc2ccc3oc4ccccc4c3c2)c1=O)NCc1cc2cnccn2c1] | 2-(5-((dibenzo[b,d]furan-2-yl- methyl)amino)-6-oxo-2-phen- ylpyrimidin-1(6H)-yl)-N-(pyr- rolo[1,2-a]pyrazin-7-ylmeth- yl)acetamide |
| 145 | 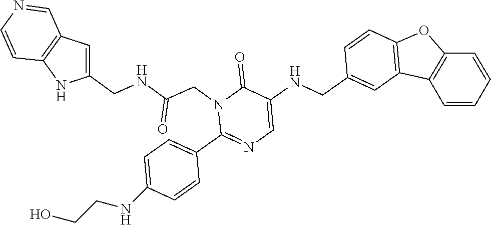 [SMILES: O=C(Cn1c(-c2ccc(NCCO)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(4-((2-hydroxyethyl)amino)- phenyl)-6-oxopyrimidin-1(6H)- yl)acetamide |
| 146 | 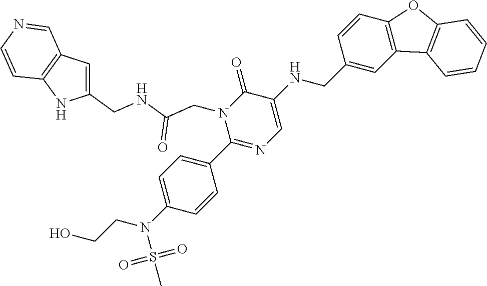 [SMILES: CS(=O)(=O)N(CCO)c1ccc(-c2ncc(NCc3ccc4oc5ccccc5c4c3)c(=O)n2CC(=O)NCc2cc3cnccc3[nH]2)cc1] | N-((1H-pyrrolo[3,2-c]pyridin- 2-yl)methyl)-2-(5-((dibenzo [b,d]furan-2-ylmethyl)amino)- 2-(4-(N-(2-hydroxyethyl)meth- ylsulfonamido)phenyl)-6-oxo- pyrimidin-1(6H)-yl)acetamide |
| 147 | 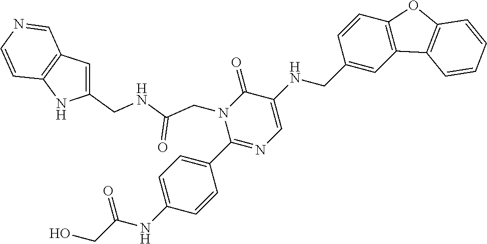 [SMILES: O=C(Cn1c(-c2ccc(NC(=O)CO)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(4-(2-hydroxyacetamido)- phenyl)-6-oxopyrimidin-1(6H)- yl)acetamide |
| 148 | 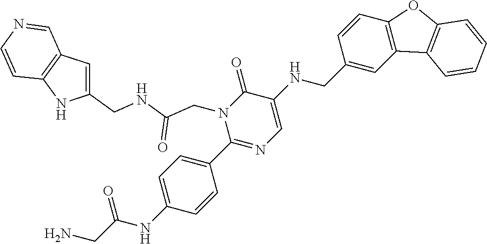 [SMILES: NCC(=O)Nc1ccc(-c2ncc(NCc3ccc4oc5ccccc5c4c3)c(=O)n2CC(=O)NCc2cc3cnccc3[nH]2)cc1] | N-((1H-pyrrolo[3,2-c]pyridin- 2-yl)methyl)-2-(2-(4-(2-amino- acetamido)phenyl)-5-((dibenzo [b,d]furan-2-ylmethyl)amino)- 6-oxopyrimidin-1(6H)-yl)- acetamide |
| 149 | 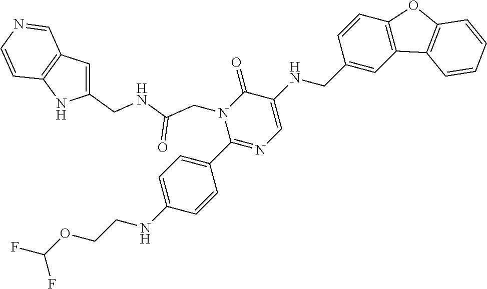 [SMILES: O=C(Cn1c(-c2ccc(NCCOC(F)F)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(4-((2-(difluoromethoxy)- ethyl)amino)phenyl)-6-oxo- pyrimidin-1(6H)-yl)acetamide |
| 150 | 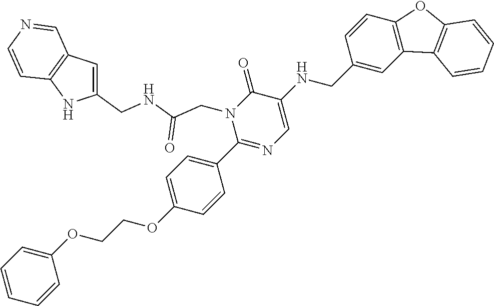 [SMILES: O=C(Cn1c(-c2ccc(OCCOc3ccccc3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(4-(2-phenoxyethoxy)- phenyl)pyrimidin-1(6H)-yl)- acetamide |
| 151 | 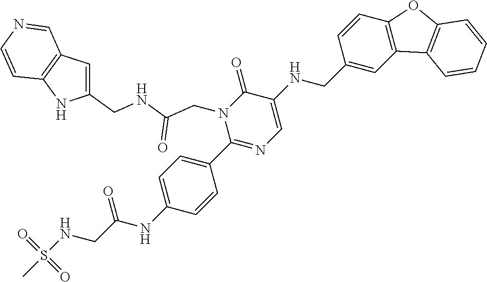 [SMILES: CS(=O)(=O)NCC(=O)Nc1ccc(-c2ncc(NCc3ccc4oc5ccccc5c4c3)c(=O)n2CC(=O)NCc2cc3cnccc3[nH]2)cc1] | N-((1H-pyrrolo[3,2-c]pyridin- 2-yl)methyl)-2-(5-((dibenzo [b,d]furan-2-ylmethyl)amino)- 2-(4-(2-(methylsulfonamido- acetamido)phenyl)-6-oxopyr- imidin-1(6H)-yl)acetamide |
| 152 | 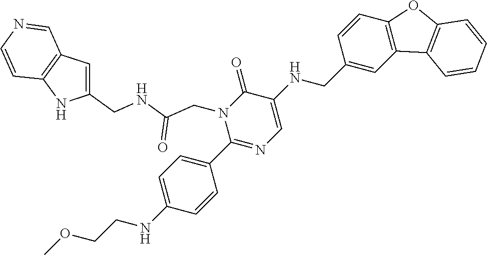 [SMILES: COCCNc1ccc(-c2ncc(NCc3ccc4oc5ccccc5c4c3)c(=O)n2CC(=O)NCc2cc3cnccc3[nH]2)cc1] | N-((1H-pyrrolo[3,2-c]pyridin- 2-yl)methyl)-2-(5-((dibenzo [b,d]furan-2-ylmethyl)amino)- 2-(4-((2-methoxyethyl)amino)- phenyl)-6-oxopyrimidin-1(6H)- yl)acetamide |
| 153 | 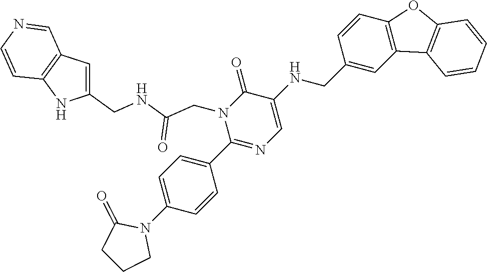 [SMILES: O=C(Cn1c(-c2ccc(N3CCCC3=O)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(4-(2-oxopyrrolidin-1- yl)phenyl)pyrimidin-1(6H)- yl)acetamide |
| 154 | 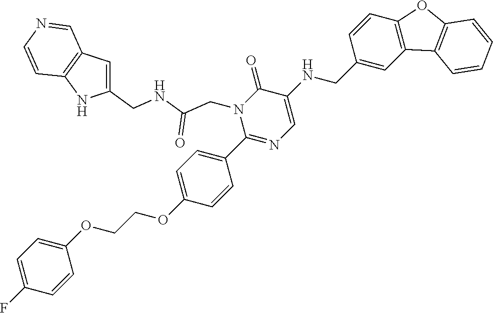 [SMILES: O=C(Cn1c(-c2ccc(OCCOc3ccc(F)cc3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(4-(2-(4-fluorophenoxy)- ethoxy)phenyl)-6-oxopyrimi- din-1(6H)-yl)acetamide |
| 155 | 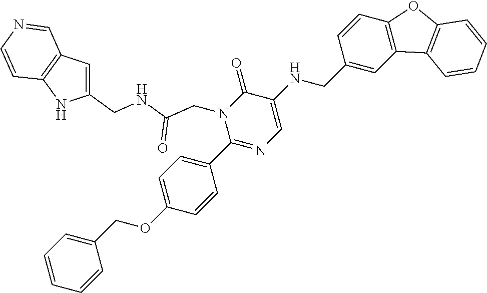 [SMILES: O=C(Cn1c(-c2ccc(OCc3ccccc3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(2-(4-(benzyl- oxy)phenyl)-5-((dibenzo[b,d] furan-2-ylmethyl)amino)-6- oxopyrimidin-1(6H)-yl)acet- amide |
| 156 | 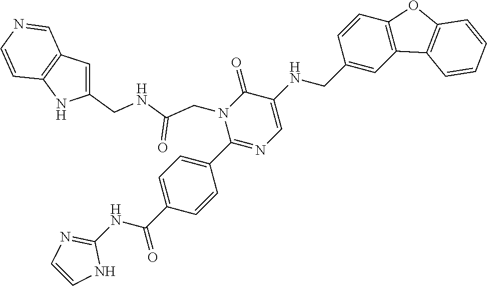 [SMILES: O=C(Cn1c(-c2ccc(C(=O)Nc3ncc[nH]3)cc2)ncc(NCc2ccc3oc4ccccc4c3c2)c1=O)NCc1cc2cnccc2[nH]1] | 4-(1-(2-(((1H-pyrrolo[3,2-c] pyridin-2-yl)methyl)amino)- 2-oxoethyl)-5-((dibenzo[b,d] furan-2-ylmethyl)amino)-6- oxo-1,6-dihydropyrimidin-2- yl)-N-(1H-imidazol-2-yl)benz- amide |
| 157 | 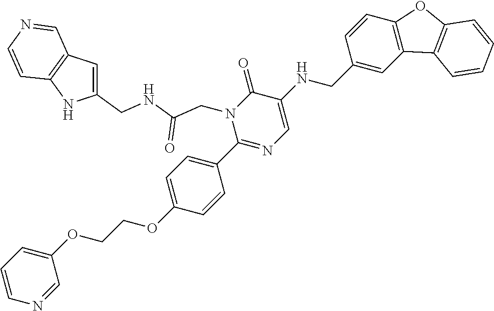 [SMILES: O=C(Cn1c(-c2ccc(OCCOc3cccnc3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(4-(2-(pyridin-3-yl- oxy)ethoxy)phenyl)pyrimidin- 1(6H)-yl)acetamide |
| 158 | 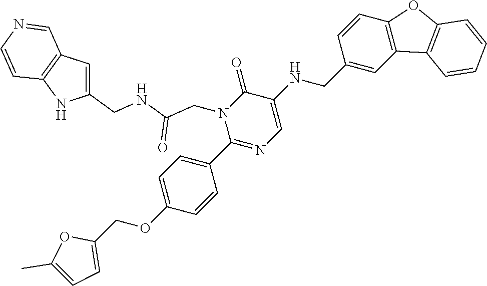 [SMILES: Cc1ccc(COc2ccc(-c3ncc(NCc4ccc5oc6ccccc6c5c4)c(=O)n3CC(=O)NCc3cc4cnccc4[nH]3)cc2)o1] | N-((1H-pyrrolo[3,2-c]pyridin- 2-yl)methyl)-2-(5-((dibenzo [b,d]furan-2-ylmethyl)amino)- 2-(4-((5-methylfuran-2-yl)- methoxy)phenyl)-6-oxopyr- imidin-1(6H)-yl)acetamide |
| 159 | 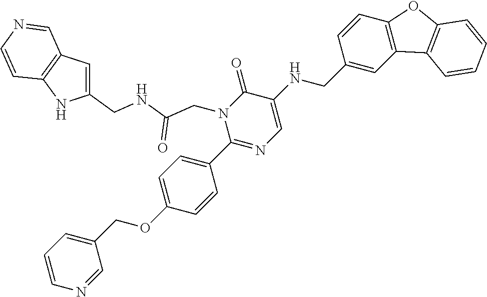 [SMILES: O=C(Cn1c(-c2ccc(OCc3cccnc3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(4-(pyridin-3-ylmeth- oxy)phenyl)pyrimidin-1(6H)- yl)acetamide |
| 160 | 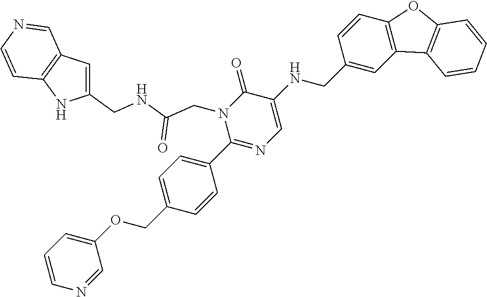 [SMILES: O=C(Cn1c(-c2ccc(COc3cccnc3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(4-((pyridin-3-yloxy)- methyl)phenyl)pyrimidin- 1(6H)-yl)acetamide |
| 161 | 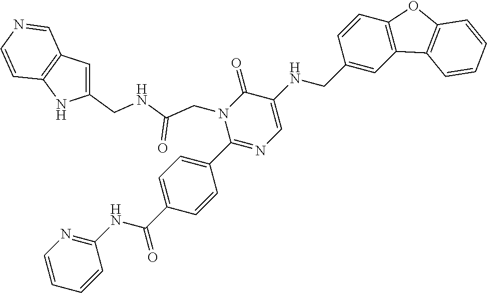 [SMILES: O=C(Cn1c(-c2ccc(C(=O)Nc3ccccn3)cc2)ncc(NCc2ccc3oc4ccccc4c3c2)c1=O)NCc1cc2cnccc2[nH]1] | 4-(1-(2-(((1H-pyrrolo[3,2-c] pyridin-2-yl)methyl)amino)-2- oxoethyl)-5-((dibenzo[b,d] furan-2-ylmethyl)amino)-6- oxo-1,6-dihydropyrimidin-2- yl)-N-(pyridin-2-yl)benzamide |
| 162 | 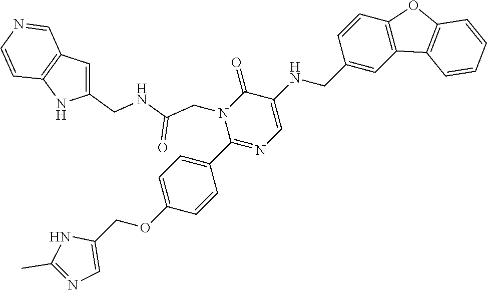 [SMILES: Cc1ncc(COc2ccc(-c3ncc(NCc4ccc5oc6ccccc6c5c4)c(=O)n3CC(=O)NCc3cc4cnccc4[nH]3)cc2)[nH]1] | N-((1H-pyrrolo[3,2-c]pyridin- 2-yl)methyl)-2-(5-((dibenzo [b,d]furan-2-ylmethyl)amino)- 2-(4-((2-methyl-1H-imidazol- 5-yl)methoxy)phenyl)-6-oxo- pyrimidin-1(6H)-yl)acetamide |
| 163 | 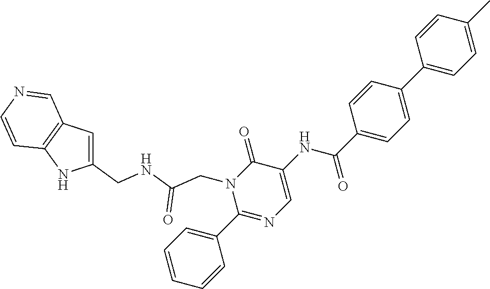 [SMILES: Cc1ccc(-c2ccc(C(=O)Nc3cnc(-c4ccccc4)n(CC(=O)NCc4cc5cnccc5[nH]4)c3=O)cc2)cc1] | N-(1-(2-(((1H-pyrrolo[3,2-c] pyridin-2-yl)methyl)amino)-2- oxoethyl)-6-oxo-2-phenyl- 1,6-dihydropyrimidin-5-yl)- 4′-methyl-[1,1′-biphenyl]-4- carboxamide |
| 164 | 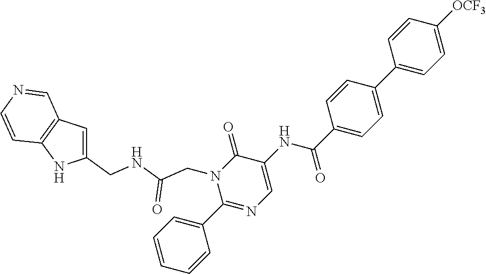 [SMILES: O=C(Cn1c(-c2ccccc2)ncc(NC(=O)c2ccc(-c3ccc(OC(F)(F)F)cc3)cc2)c1=O)NCc1cc2cnccc2[nH]1] | N-(1-(2-(((1H-pyrrolo[3,2-c] pyridin-2-yl)methyl)amino)- 2-oxoethyl)-6-oxo-2-phenyl- 1,6-dihydropyrimidin-5-yl)- 4′-(trifluoromethoxy)-[1,1′- biphenyl]-4-carboxamide |
| 165 | 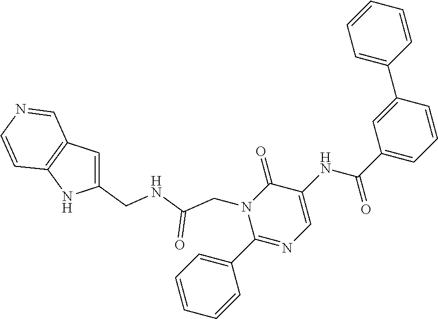 [SMILES: O=C(Cn1c(-c2ccccc2)ncc(NC(=O)c2cccc(-c3ccccc3)c2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-[1,1′- biphenyl]-3-carboxamide |
| 166 | 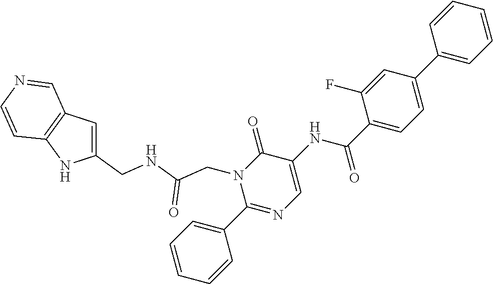 [SMILES: O=C(Cn1c(-c2ccccc2)ncc(NC(=O)c2ccc(-c3ccccc3)cc2F)c1=O)NCc1cc2cnccc2[nH]1] | N-(1-(2-(((1H-pyrrolo[3,2-c] pyridin-2-yl)methyl)amino)- 2-oxoethyl)-6-oxo-2-phenyl- 1,6-dihydropyrimidin-5-yl)- 3-fluoro-[1,1′-biphenyl]-4- carboxamide |
| 167 | 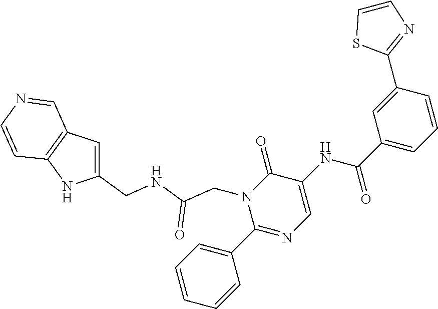 [SMILES: O=C(Cn1c(-c2ccccc2)ncc(NC(=O)c2cccc(-c3nccs3)c2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-3- (thiazol-2-yl)benzamide |
| 168 | 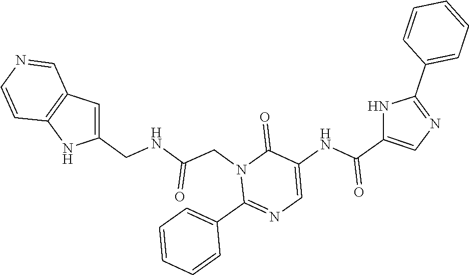 [SMILES: O=C(Cn1c(-c2ccccc2)ncc(NC(=O)c2cnc(-c3ccccc3)[nH]2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2- phenyl-1H-imidazole-5-carbox- amide |
| 169 | 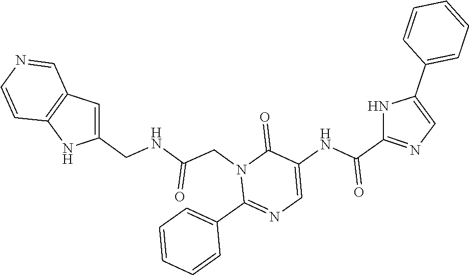 [SMILES: O=C(Cn1c(-c2ccccc2)ncc(NC(=O)c2ncc(-c3ccccc3)[nH]2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- yl-1H-imidazole-2-carbox- amide |
| 170 | 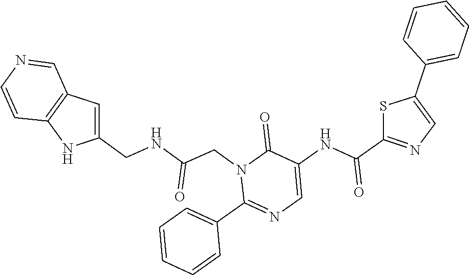 [SMILES: O=C(Cn1c(-c2ccccc2)ncc(NC(=O)c2ncc(-c3ccccc3)s2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- ylthiazole-2-carboxamide |
| 171 | 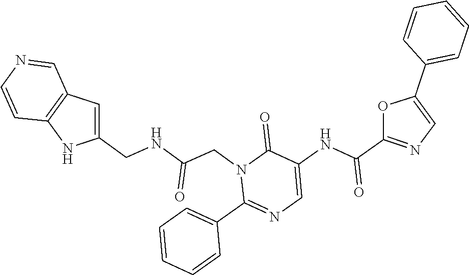 [SMILES: O=C(Cn1c(-c2ccccc2)ncc(NC(=O)c2ncc(-c3ccccc3)o2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- yloxazole-2-carboxamide |
| 172 | 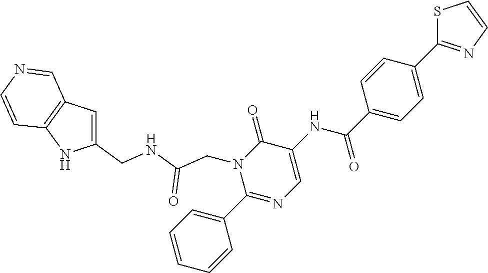 [SMILES: O=C(Cn1c(-c2ccccc2)ncc(NC(=O)c2ccc(-c3nccs3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4- (thiazol-2-yl)benzamide |
| 173 | 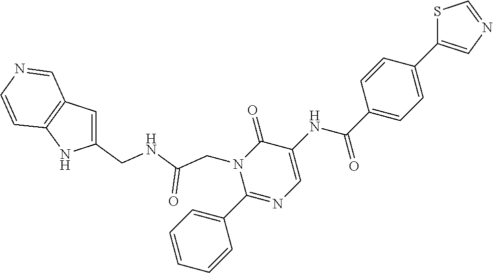 [SMILES: O=C(Cn1c(-c2ccccc2)ncc(NC(=O)c2ccc(-c3cncs3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4- (thiazol-5-yl)benzamide |
| 174 | 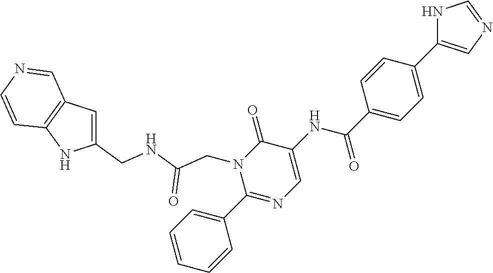 [SMILES: O=C(Cn1c(-c2ccccc2)ncc(NC(=O)c2ccc(-c3cnc[nH]3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(1H- imidazol-5-yl)benzamide |
| 175 | 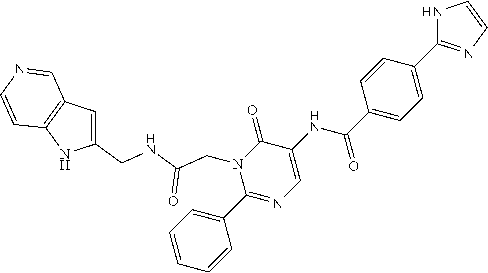 [SMILES: O=C(Cn1c(-c2ccccc2)ncc(NC(=O)c2ccc(-c3ncc[nH]3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-(1H- imidazol-2-yl)benzamide |
| 176 | 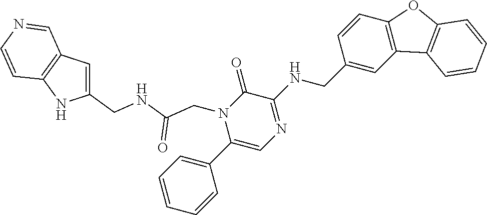 [SMILES: O=C(Cn1c(-c2ccccc2)cnc(NCc2ccc3oc4ccccc4c3c2)c1=O)NCc1cc2cnccc2[nH]1] | N-((1H-pyrrolo[3,2-c]pyridin- 2-yl)methyl)-2-(3-((dibenzo [b,d]furan-2-ylmethyl)amino)- 2-oxo-6-phenylpyrazin-1(2H)- yl)acetamide |
| 177 | 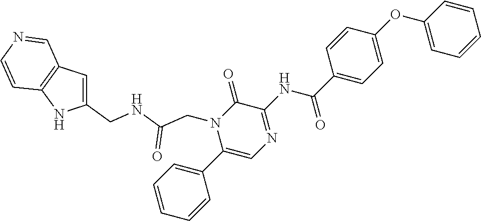 [SMILES: O=C(Cn1c(-c2ccccc2)cnc(NC(=O)c2ccc(Oc3ccccc3)cc2)c1=O)NCc1cc2cnccc2[nH]1] | N-(4-(2-(((1H-pyrrolo[3,2-c] pyridin-2-yl)methyl)amino)-2- oxoethyl)-3-oxo-5-phenyl-3,4- dihydropyrazin-2-yl)-4-phen- oxybenzamide |
| 178 | 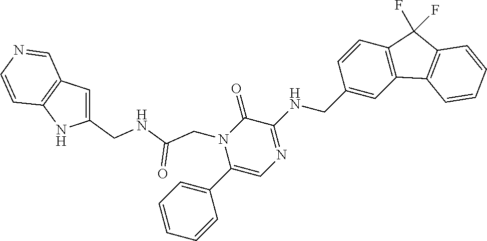 [SMILES: O=C(Cn1c(-c2ccccc2)cnc(NCc2ccc3c(c2)-c2ccccc2C3(F)F)c1=O)NCc1cc2cnccc2[nH]1] | N-((1H-pyrrolo[3,2-c]pyridin- 2-yl)methyl)-2-(3-(((9,9- difluoro-9H-fluoren-3-yl)- methyl)amino)-2-oxo-6-phen- ylpyrazin-1(2H)-yl)acetamide |
| 179 | 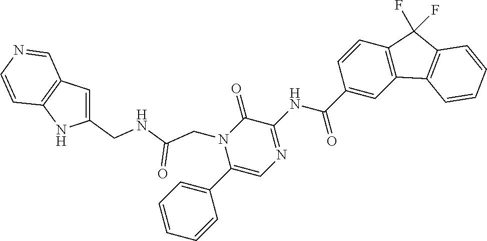 [SMILES: O=C(Cn1c(-c2ccccc2)cnc(NC(=O)c2ccc3c(c2)-c2ccccc2C3(F)F)c1=O)NCc1cc2cnccc2[nH]1] | N-(4-(2-(((1H-pyrrolo[3,2-c] pyridin-2-yl)methyl)amino)- 2-oxoethyl)-3-oxo-5-phenyl- 3,4-dihydropyrazin-2-yl)-9,9- difluoro-9H-fluorene-3-carbox- amide |
| 180 | 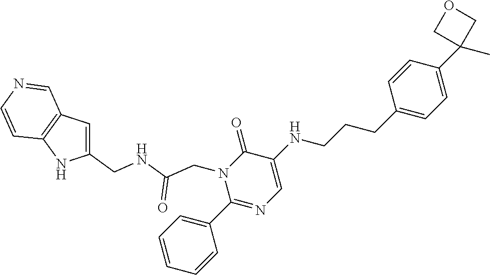 [SMILES: CC1(c2ccc(CCCNc3cnc(-c4ccccc4)n(CC(=O)NCc4cc5cnccc5[nH]4)c3=O)cc2)COC1] | N-((1H-pyrrolo[3,2-c]pyridin- 2-yl)methyl)-2-(5-((3-(4-(3- methyloxetan-3-yl)phenyl)- propyl)amino)-6-oxo-2-phenyl- pyrimidin-1(6H)-yl)acetamide |
| 181 | 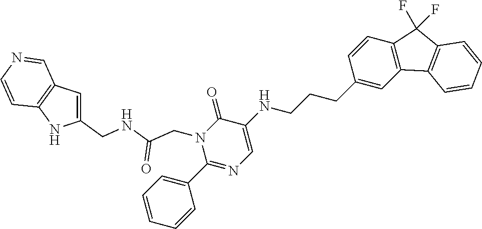 [SMILES: O=C(Cn1c(-c2ccccc2)ncc(NCCCc2ccc3c(c2)-c2ccccc2C3(F)F)c1=O)NCc1cc2cnccc2[nH]1] | N-((1H-pyrrolo[3,2-c]pyridin- 2-yl)methyl)-2-(5-((3-(9,9- difluoro-9H-fluoren-3-yl)prop- yl)amino)-6-oxo-2-phenylpyr- imidin-1(6H)-yl)acetamide |
| 182 | 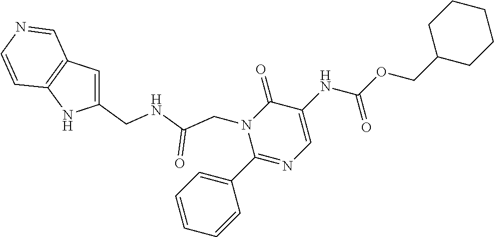 [SMILES: O=C(Cn1c(-c2ccccc2)ncc(NC(=O)OCC2CCCCC2)c1=O)NCc1cc2cnccc2[nH]1] | Cyclohexylmethyl (1-(2-(((1H- pyrrolo[3,2-c]pyridin-2-yl)- methyl)amino)-2-oxoethyl)-6- oxo-2-phenyl-1,6-dihydropyr- imidin-5-yl)carbamate |
| 183 | 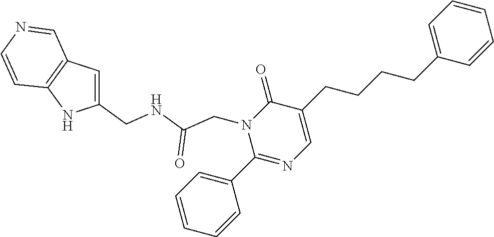 [SMILES: O=C(Cn1c(-c2ccccc2)ncc(CCCCc2ccccc2)c1=O)NCc1cc2cnccc2[nH]1] | N-((1H-pyrrolo[3,2-c]pyridin- 2-yl)methyl)-2-(6-oxo-2-phen- yl-5-(4-phenylbutyl)pyrimidin- 1(6H)-yl)acetamide |
| 184 | 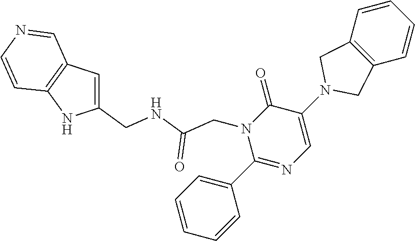 [SMILES: O=C(Cn1c(-c2ccccc2)ncc(N2Cc3ccccc3C2)c1=O)NCc1cc2cnccc2[nH]1] | N-((1H-pyrrolo[3,2-c]pyridin- 2-yl)methyl)-2-(5-(isoindolin- 2-yl)-6-oxo-2-phenylpyrimi- din-1(6H)-yl)acetamide |
| 185 | 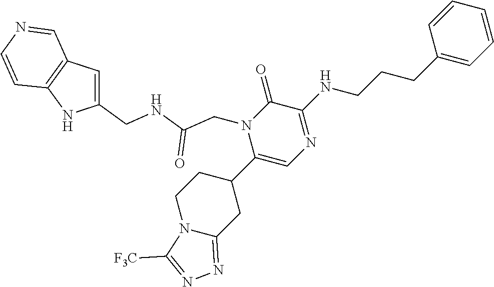 [SMILES: O=C(Cn1c(C2CCn3c(nnc3C(F)(F)F)C2)cnc(NCCCc2ccccc2)c1=O)NCc1cc2cnccc2[nH]1] | N-((1H-pyrrolo[3,2-c]pyridin- 2-yl)methyl)-2-(2-oxo-3-((3- phenylpropyl)amino)-6-(3- (trifluoromethyl)-5,6-dihydro- [1,2,4]triazolo[4,3-a]pyrazin- 7(8H)-yl)pyrazin-1(2H)-yl)- acetamide |
| 186 | 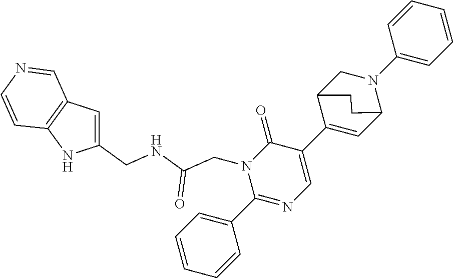 [SMILES: O=C(Cn1c(-c2ccccc2)ncc(C2=CC3CCC2CN3c2ccccc2)c1=O)NCc1cc2cnccc2[nH]1] | N-((1H-pyrrolo[3,2-c]pyridin- 2-yl)methyl)-2-(6-oxo-2-phen- yl-5-(2-phenyl-2-azabicyclo [2.2.2]oct-5-en-5-yl)pyrimidin- 1(6H)-yl)acetamide |
| 187 | 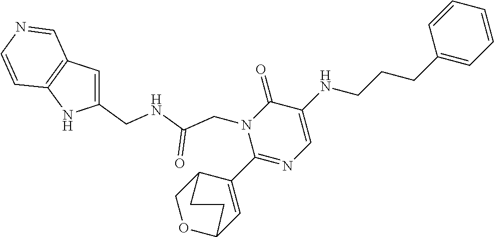 [SMILES: O=C(Cn1c(C2=CC3CCC2CO3)ncc(NCCCc2ccccc2)c1=O)NCc1cc2cnccc2[nH]1] | N-((1H-pyrrolo[3,2-c]pyridin- 2-yl)methyl)-2-(2-(2-oxabi- cyclo[2.2.2]oct-5-en-5-yl)- 6-oxo-5-((3-phenylpropyl)- amino)-pyrimidin-1(6H)-yl)- acetamide |
| 188 | 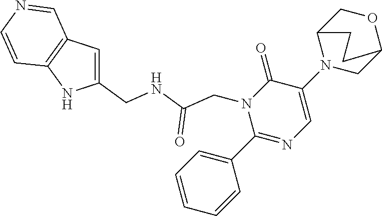 [SMILES: O=C(Cn1c(-c2ccccc2)ncc(N2CC3CCC2CO3)c1=O)NCc1cc2cnccc2[nH]1] | N-((1H-pyrrolo[3,2-c]pyridin- 2-yl)methyl)-2-(5-(2-oxa-5- azabicyclo[2.2.2]octan-5-yl)- 6-oxo-2-phenylpyrimidin- 1(6H)-yl)acetamide |
| 189 | 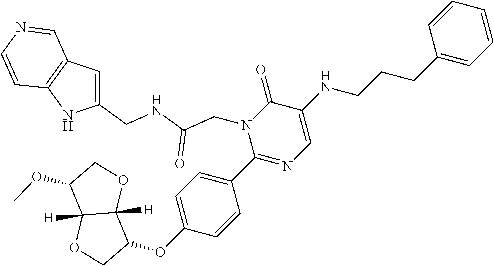 | N-((1H-pyrrolo[3,2-c]pyridin- 2-yl)methyl)-2-(2-(4-(((3R, 3aR,6R,6aR)-6-methoxyhexa- hydrofuro[3,2-b]furan-3-yl)- oxy)phenyl)-6-oxo-5-((3-phen- ylpropyl)amino)pyrimidin- 1(6H)-yl)acetamide |
| 190 | 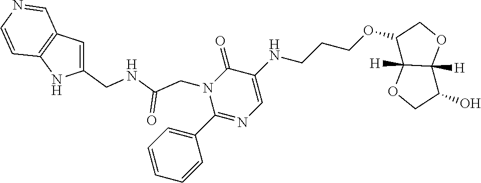 | N-((1H-pyrrolo[3,2-c]pyridin- 2-yl)methyl)-2-(5-((3-(((3R, 3aR,6R,6aR)-6-hydroxyhexa- hydrofuro[3,2-b]furan-3-yl)- oxy)propyl)amino)-6-oxo-2- phenylpyrimidin-1(6H)-yl)- acetamide |
| 191 | 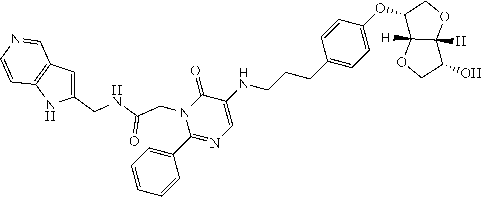 [SMILES: O=C(Cn1c(-c2ccccc2)ncc(NCCCc2ccc(O[C@@H]3CO[C@H]4[C@@H]3OC[C@H]4O)cc2)c1=O)NCc1cc2cnccc2[nH]1] | N-((1H-pyrrolo[3,2-c]pyridin- 2-yl)methyl)-2-(5-((3-(4-(((3R, 3aR,6R,6aR)-6-hydroxyhexa- hydrofuro[3,2-b]furan-3-yl)- oxy)phenyl)propyl)amino)-6- oxo-2-phenylpyrimidin-1(6H)- yl)acetamide |
| 192 | 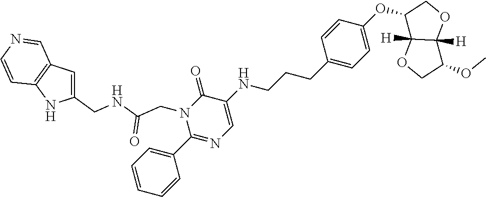 [SMILES: CO[C@@H]1CO[C@H]2[C@@H]1OC[C@H]2Oc1ccc(CCCNc2cnc(-c3ccccc3)n(CC(=O)NCc3cc4cnccc4[nH]3)c2=O)cc1] | N-((1H-pyrrolo[3,2-c]pyridin- 2-yl)methyl)-2-(5-((3-(4-(((3R, 3aR,6R,6aR)-6-methoxyhexa- hydrofuro[3,2-b]furan-3-yl)- oxy)phenyl)propyl)amino)-6- oxo-2-phenylpyrimidin-1(6H)- yl)acetamide |
| 193 | 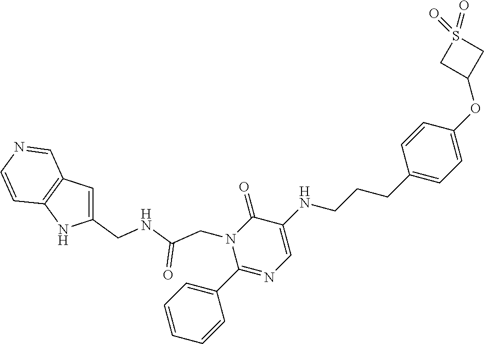 [SMILES: O=C(Cn1c(-c2ccccc2)ncc(NCCCc2ccc(OC3CS(=O)(=O)C3)cc2)c1=O)NCc1cc2cnccc2[nH]1] | N-((1H-pyrrolo[3,2-c]pyridin- 2-yl)methyl)-2-(5-((3-(4-((1,1- dioxidothietan-3-yl)oxy)phen- yl)propyl)amino)-6-oxo-2- phenylpyrimidin-1(6H)-yl)- acetamide |
| 194 | 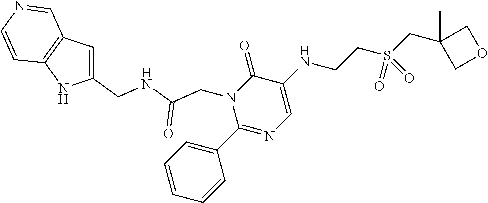 [SMILES: CC1(CS(=O)(=O)CCNc2cnc(-c3ccccc3)n(CC(=O)NCc3cc4cnccc4[nH]3)c2=O)COC1] | N-((1H-pyrrolo[3,2-c]pyridin- 2-yl)methyl)-2-(5-((2-(((3- methyloxetan-3-yl)methyl)- sulfonyl)ethyl)amino)-6-oxo- 2-phenylpyrimidin-1(6H)-yl)- acetamide |
| 195 | 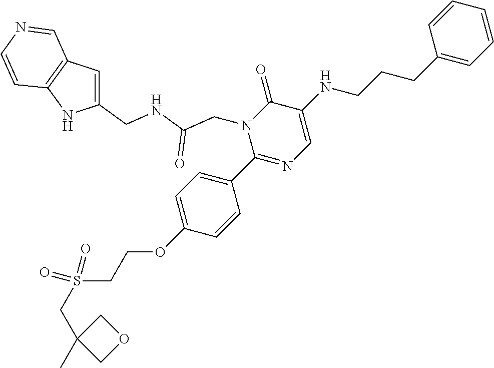 [SMILES: CC1(CS(=O)(=O)CCOc2ccc(-c3ncc(NCCCc4ccccc4)c(=O)n3CC(=O)NCc3cc4cnccc4[nH]3)cc2)COC1] | N-((1H-pyrrolo[3,2-c]pyridin- 2-yl)methyl)-2-(2-(4-(2-(((3- methyloxetan-3-yl)methyl)- sulfonyl)ethoxy)phenyl)-6-oxo- 5-((3-phenylpropyl)amino)pyr- imidin-1(6H)-yl)acetamide |
| 196 | 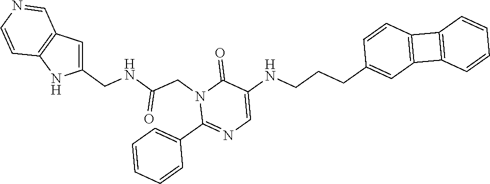 [SMILES: O=C(Cn1c(-c2ccccc2)ncc(NCCCc2ccc3c(c2)-c2ccccc2-3)c1=O)NCc1cc2cnccc2[nH]1] | N-((1H-pyrrolo[3,2-c]pyridin- 2-yl)methyl)-2-(5-((3-(biphen- ylen-2-yl)propyl)amino)-6-oxo- 2-phenylpyrimidin-1(6H)-yl)- acetamide |
| 197 | 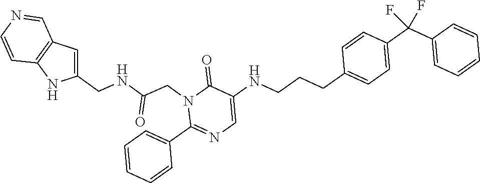 [SMILES: O=C(Cn1c(-c2ccccc2)ncc(NCCCc2ccc(C(F)(F)c3ccccc3)cc2)c1=O)NCc1cc2cnccc2[nH]1] | N-((1H-pyrrolo[3,2-c]pyridin- 2-yl)methyl)-2-(5-((3-(4- (difluoro(phenyl)methyl)- phenyl)propyl)amino)-6-oxo- 2-phenylpyrimidin-1(6H)-yl)- acetamide |
| 198 | 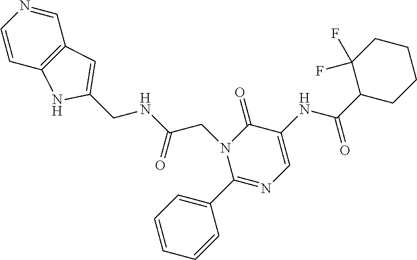 [SMILES: O=C(Cn1c(-c2ccccc2)ncc(NC(=O)C2CCCCC2(F)F)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2,2- difluorocyclohexane-1-carbox- amide |
| 199 | 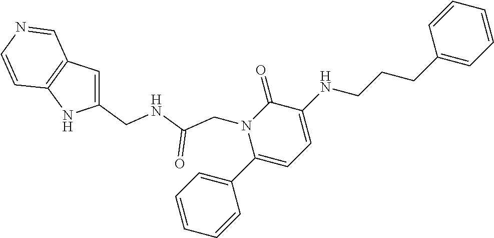 [SMILES: O=C(Cn1c(-c2ccccc2)ccc(NCCCc2ccccc2)c1=O)NCc1cc2cnccc2[nH]1] | N-((1H-pyrrolo[3,2-c]pyridin- 2-yl)methyl)-2-(2-oxo-6-phen- yl-3-((3-phenylpropyl)amino)- pyridin-1(2H)-yl)acetamide |
| 200 | 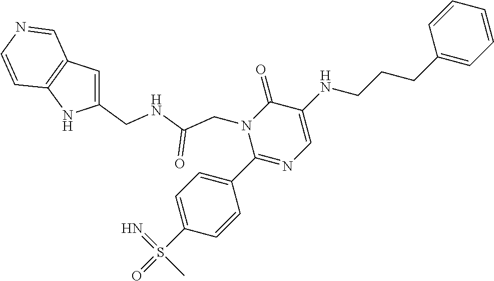 [SMILES: CS(=N)(=O)c1ccc(-c2ncc(NCCCc3ccccc3)c(=O)n2CC(=O)NCc2cc3cnccc3[nH]2)cc1] | N-((1H-pyrrolo[3,2-c]pyridin- 2-yl)methyl)-2-(2-(4-(S-meth- ylsulfonimidoyl)phenyl)-6-oxo- 5-((3-phenylpropyl)amino)pyr- imidin-1(6H)-yl)acetamide |
| 201 | 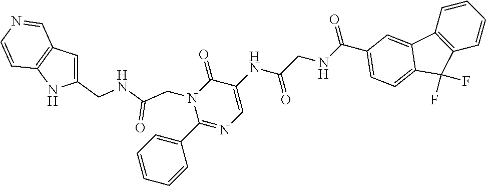 [SMILES: O=C(Cn1c(-c2ccccc2)ncc(NC(=O)CNC(=O)c2ccc3c(c2)-c2ccccc2C3(F)F)c1=O)NCc1cc2cnccc2[nH]1] | N-(2-((1-(2-(((1H-pyrrolo[3,2- c]pyridin-2-yl)methyl)amino)- 2-oxoethyl)-6-oxo-2-phenyl- 1,6-dihydropyrimidin-5-yl)- amino)-2-oxoethyl)-9,9- difluoro-9H-fluorene-3-car- boxamide |
| 202 | 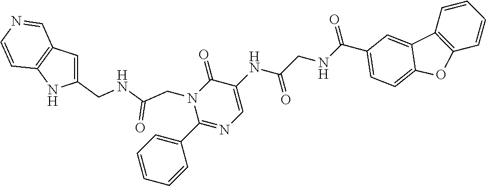 [SMILES: O=C(Cn1c(-c2ccccc2)ncc(NC(=O)CNC(=O)c2ccc3oc4ccccc4c3c2)c1=O)NCc1cc2cnccc2[nH]1] | N-(2-((1-(2-(((1H-pyrrolo[3,2- c]pyridin-2-yl)methyl)amino)- 2-oxoethyl)-6-oxo-2-phenyl- 1,6-dihydropyrimidin-5-yl)- amino)-2-oxoethyl)dibenzo [b,d]furan-2-carboxamide |
| 203 | 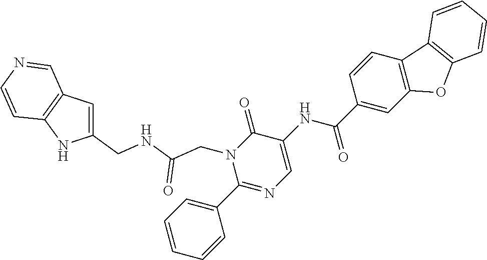 [SMILES: O=C(Cn1c(-c2ccccc2)ncc(NC(=O)c2ccc3c(c2)oc2ccccc23)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)dibenzo [b,d]furan-3-carboxamide |
| 204 | 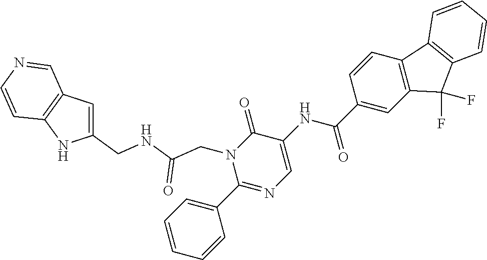 [SMILES: O=C(Cn1c(-c2ccccc2)ncc(NC(=O)c2ccc3c(c2)C(F)(F)c2ccccc2-3)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-9,9- difluoro-9H-fluorene-2-carbox- amide |
| 205 | 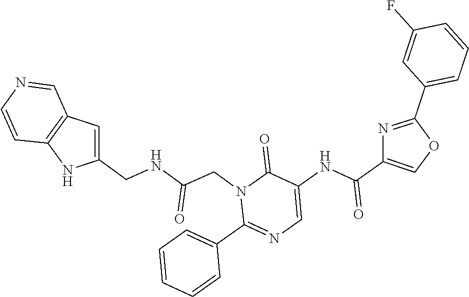 [SMILES: O=C(Cn1c(-c2ccccc2)ncc(NC(=O)c2coc(-c3cccc(F)c3)n2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2-(3- fluorophenyl)oxazole-4-carbox- amide |
| 206 | 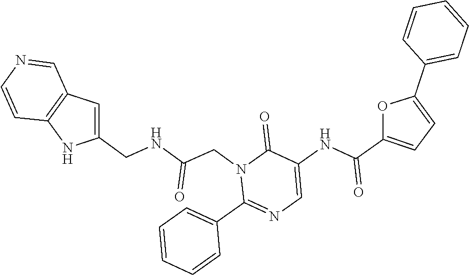 [SMILES: O=C(Cn1c(-c2ccccc2)ncc(NC(=O)c2ccc(-c3ccccc3)o2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- ylfuran-2-carboxamide |
| 207 | 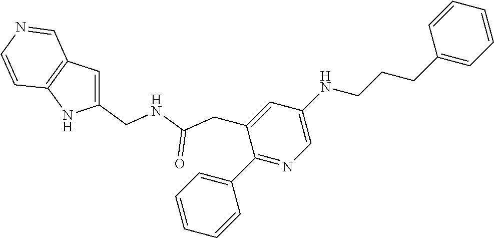 [SMILES: O=C(Cc1cc(NCCCc2ccccc2)cnc1-c1ccccc1)NCc1cc2cnccc2[nH]1] | N-((1H-pyrrolo[3,2-c]pyridin- 2-yl)methyl)-2-(2-phenyl-5-((3- phenylpropyl)amino)pyridin-3- yl)acetamide |
| 208 | 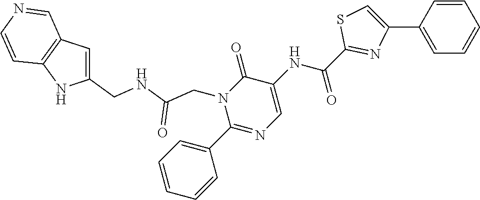 [SMILES: O=C(Cn1c(-c2ccccc2)ncc(NC(=O)c2nc(-c3ccccc3)cs2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-phen- ylthiazole-2-carboxamide |
| 209 | 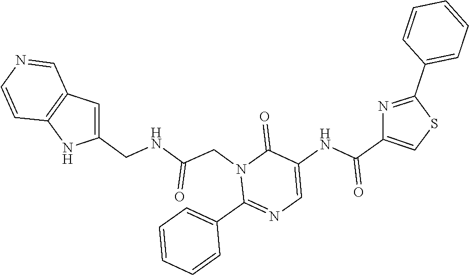 [SMILES: O=C(Cn1c(-c2ccccc2)ncc(NC(=O)c2csc(-c3ccccc3)n2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2-phen- ylthiazole-4-carboxamide |
| 210 | 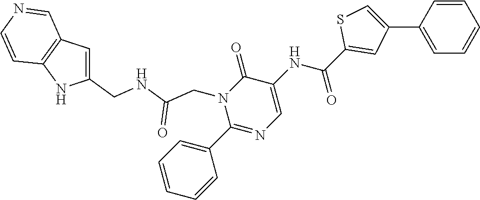 [SMILES: O=C(Cn1c(-c2ccccc2)ncc(NC(=O)c2cc(-c3ccccc3)cs2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-phen- ylthiophene-2-carboxamide |
| 211 | 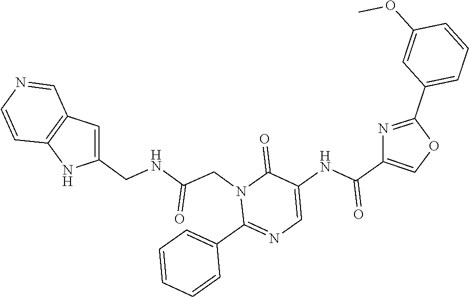 [SMILES: COc1cccc(-c2nc(C(=O)Nc3cnc(-c4ccccc4)n(CC(=O)NCc4cc5cnccc5[nH]4)c3=O)co2)c1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2-(3- methoxyphenyl)oxazole-4- carboxamide |
| 212 | 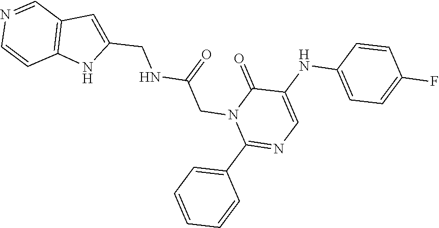 [SMILES: O=C(Cn1c(-c2ccccc2)ncc(Nc2ccc(F)cc2)c1=O)NCc1cc2cnccc2[nH]1] | N-((1H-pyrrolo[3,2-c]pyridin- 2-yl)methyl)-2-(5-((4-fluoro- phenyl)amino)-6-oxo-2-phen- ylpyrimidin-1(6H)-yl)acet- amide |
| 213 | 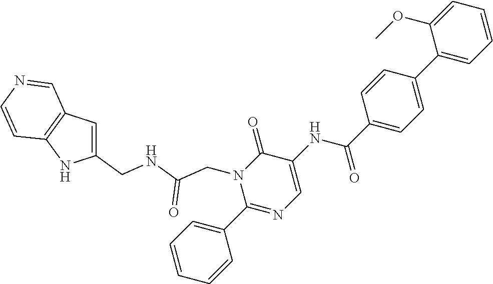 [SMILES: COc1ccccc1-c1ccc(C(=O)Nc2cnc(-c3ccccc3)n(CC(=O)NCc3cc4cnccc4[nH]3)c2=O)cc1] | N-(1-(2-(((1H-pyrrolo[3,2-c] pyridin-2-yl)methyl)amino)-2- oxoethyl)-6-oxo-2-phenyl- 1,6-dihydropyrimidin-5-yl)- 2′-methoxy-[1,1′-biphenyl]-4- carboxamide |
| 214 | 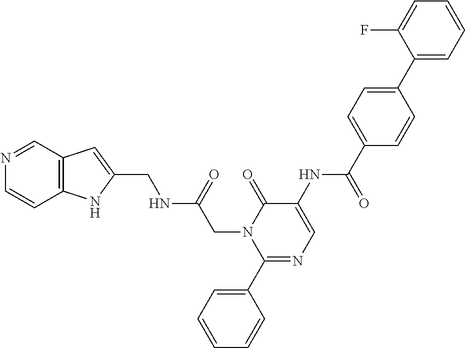 [SMILES: O=C(Cn1c(-c2ccccc2)ncc(NC(=O)c2ccc(-c3ccccc3F)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl- 1,6-dihydropyrimidin-5-yl)- 2′-fluoro-[1,1′-biphenyl]-4- carboxamide |
| 215 | 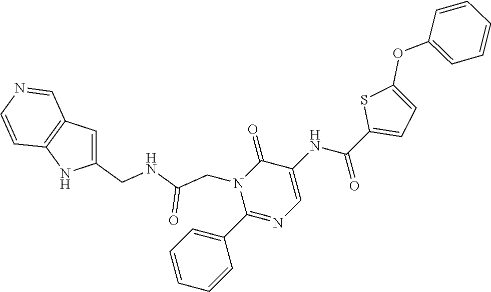 [SMILES: O=C(Cn1c(-c2ccccc2)ncc(NC(=O)c2ccc(Oc3ccccc3)s2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- oxythiophene-2-carboxamide |
| 216 | 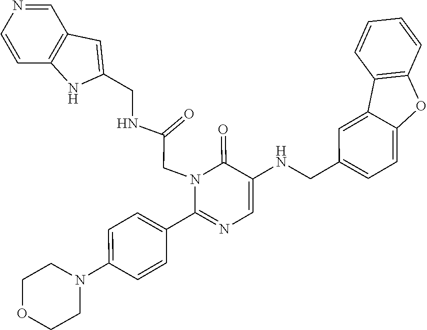 [SMILES: O=C(Cn1c(-c2ccc(N3CCOCC3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(4-morpholinophenyl)-6-oxo- pyrimidin-1(6H)-yl)acetamide |
| 217 | 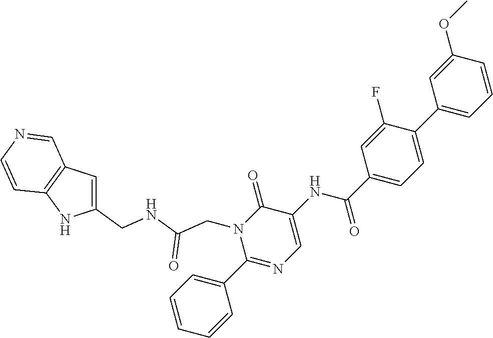 [SMILES: COc1cccc(-c2ccc(C(=O)Nc3cnc(-c4ccccc4)n(CC(=O)NCc4cc5cnccc5[nH]4)c3=O)cc2F)c1] | N-(1-(2-(((1H-pyrrolo[3,2-c] pyridin-2-yl)methyl)amino)- 2-oxoethyl)-6-oxo-2-phenyl- 1,6-dihydropyrimidin-5-yl)- 2-fluoro-3′-methoxy-[1,1′- biphenyl]-4-carboxamide |
| 218 | 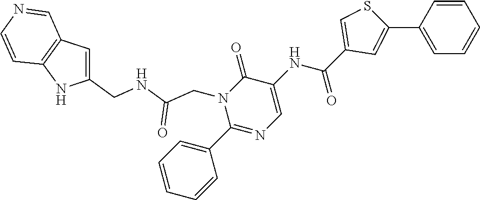 [SMILES: O=C(Cn1c(-c2ccccc2)ncc(NC(=O)c2csc(-c3ccccc3)c2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- ylthiophene-3-carboxamide |
| 219 | 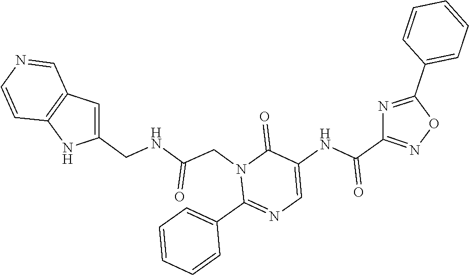 [SMILES: O=C(Cn1c(-c2ccccc2)ncc(NC(=O)c2noc(-c3ccccc3)n2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- yl-1,2,4-oxadiazole-3-carbox- amide |
| 220 | 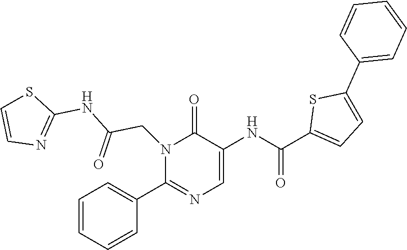 [SMILES: O=C(Cn1c(-c2ccccc2)ncc(NC(=O)c2ccc(-c3ccccc3)s2)c1=O)Nc1nccs1] | N-(6-oxo-1-(2-oxo-2-(thiazol- 2-ylamino)ethyl)-2-phenyl-1,6- dihydropyrimidin-5-yl)-5-phen- ylthiophene-2-carboxamide |
| 221 | 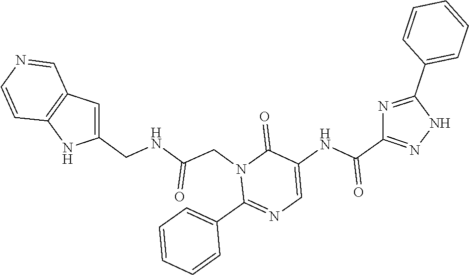 [SMILES: O=C(Cn1c(-c2ccccc2)ncc(NC(=O)c2n[nH]c(-c3ccccc3)n2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- yl-1H-1,2,4-triazole-3-carbox- amide |
| 222 | 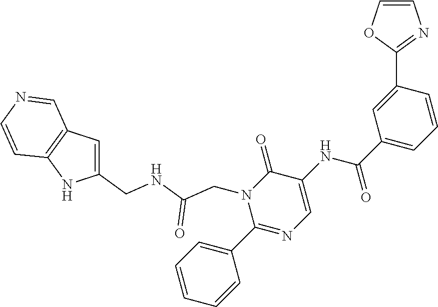 [SMILES: O=C(Cn1c(-c2ccccc2)ncc(NC(=O)c2cccc(-c3ncco3)c2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-3- (oxazol-2-yl)benzamide |
| 223 | 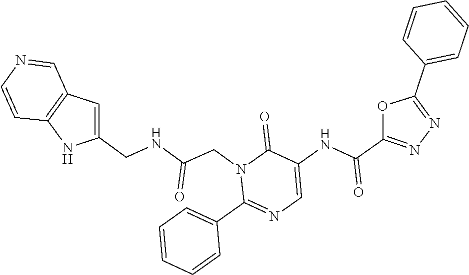 [SMILES: O=C(Cn1c(-c2ccccc2)ncc(NC(=O)c2nnc(-c3ccccc3)o2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-5-phen- yl-1,3,4-oxadiazole-2-carbox- amide |
| 224 | 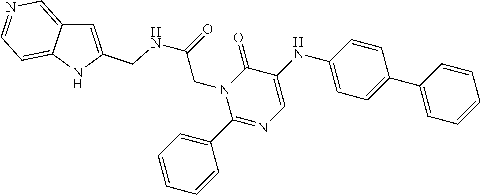 [SMILES: O=C(Cn1c(-c2ccccc2)ncc(Nc2ccc(-c3ccccc3)cc2)c1=O)NCc1cc2cnccc2[nH]1] | N-((1H-pyrrolo[3,2-c]pyridin- 2-yl)methyl)-2-(5-([1,1′- biphenyl]-4-ylamino)-6-oxo- 2-phenylpyrimidin-1(6H)-yl)- acetamide |
| 225 | 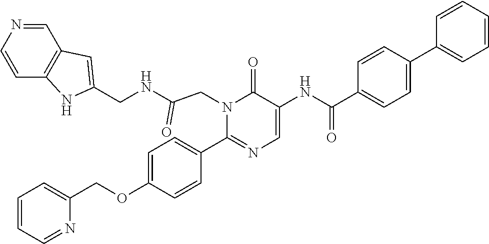 [SMILES: O=C(Cn1c(-c2ccc(OCc3ccccn3)cc2)ncc(NC(=O)c2ccc(-c3ccccc3)cc2)c1=O)NCc1cc2cnccc2[nH]1] | N-(1-(2-(((1H-pyrrolo[3,2-c] pyridin-2-yl)methyl)amino)-2- oxoethyl)-6-oxo-2-(4-(pyridin- 2-ylmethoxy)phenyl)-1,6- dihydropyrimidin-5-yl)-[1,1′- biphenyl]-4-carboxamide |
| 226 | 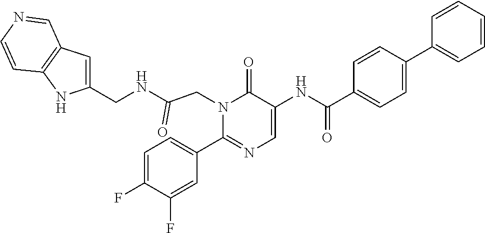 [SMILES: O=C(Cn1c(-c2ccc(F)c(F)c2)ncc(NC(=O)c2ccc(-c3ccccc3)cc2)c1=O)NCc1cc2cnccc2[nH]1] | N-(1-(2-(((1H-pyrrolo[3,2-c] pyridin-2-yl)methyl)amino)-2- oxoethyl)-2-(3,4-difluorophen- yl)-6-oxo-1,6-dihydropyrimi- din-5-yl)-[1,1′-biphenyl]-4- carboxamide |
| 227 | 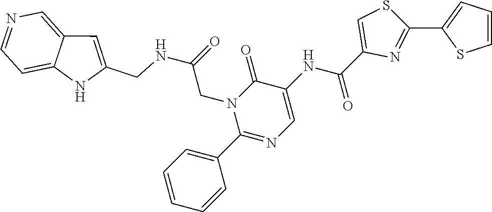 [SMILES: O=C(Cn1c(-c2ccccc2)ncc(NC(=O)c2csc(-c3cccs3)n2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-2- (thiophen-2-yl)thiazole-4- carboxamide |
| 228 | 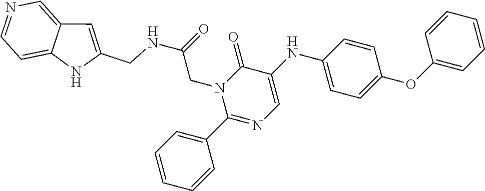 [SMILES: O=C(Cn1c(-c2ccccc2)ncc(Nc2ccc(Oc3ccccc3)cc2)c1=O)NCc1cc2cnccc2[nH]1] | N-((1H-pyrrolo[3,2-c]pyridin- 2-yl)methyl)-2-(6-oxo-5-((4- phenoxyphenyl)amino)-2- phenylpyrimidin-1(6H)-yl)- acetamide |
| 229 | 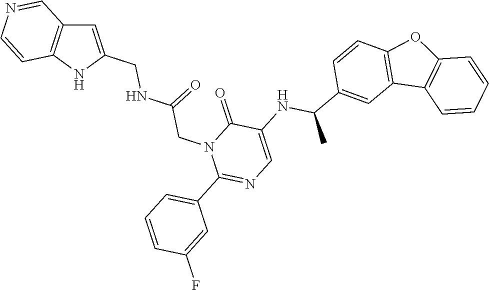 [SMILES: C[C@@H](Nc1cnc(-c2cccc(F)c2)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(5-((1- (dibenzo[b,d]furan-2-yl)ethyl)- amino)-2-(3-fluorophenyl)-6- oxopyrimidin-1(6H)-yl)acet- amide |
| 230 | 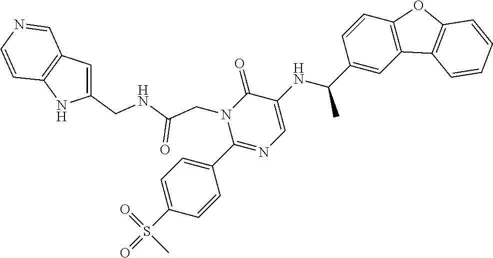 [SMILES: C[C@@H](Nc1cnc(-c2ccc(S(C)(=O)=O)cc2)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(5-((1- (dibenzo[b,d]furan-2-yl)ethyl)- amino)-2-(4-(methylsulfonyl)- phenyl)-6-oxopyrimidin-1(6H)- yl)acetamide |
| 231 | 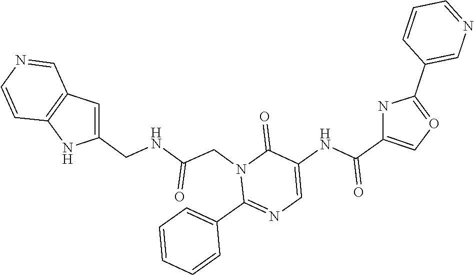 | N-(1-(2-(((1H-pyrrolo[3,2-c] pyridin-2-yl)methyl)amino)-2- oxoethyl)-6-oxo-2-phenyl-1,6- dihydropyrimidin-5-yl)-2-(pyr- idin-3-yl)oxazole-4-carbox- amide |
| 232 | 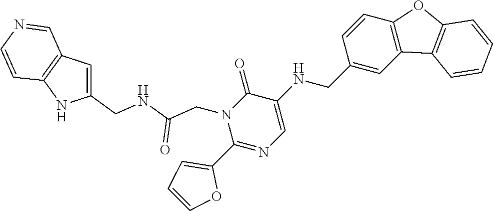 [SMILES: O=C(Cn1c(-c2ccco2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(furan-2-yl)-6-oxopyrimidin- 1(6H)-yl)acetamide |
| 233 | 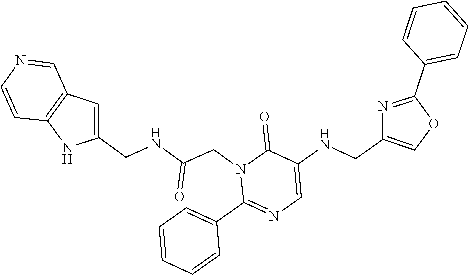 [SMILES: O=C(Cn1c(-c2ccccc2)ncc(NCc2coc(-c3ccccc3)n2)c1=O)NCc1cc2cnccc2[nH]1] | N-((1H-pyrrolo[3,2-c]pyridin- 2-yl)methyl)-2-(6-oxo-2-phen- yl-5-(((2-phenyloxazol-4-yl)- methyl)amino)pyrimidin- 1(6H)-yl)acetamide |
| 234 | 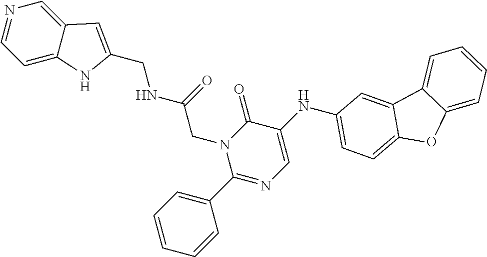 [SMILES: O=C(Cn1c(-c2ccccc2)ncc(Nc2ccc3oc4ccccc4c3c2)c1=O)NCc1cc2cnccc2[nH]1] | N-((1H-pyrrolo[3,2-c]pyridin- 2-yl)methyl)-2-(5-(dibenzo [b,d]furan-2-ylamino)-6-oxo- 2-phenylpyrimidin-1(6H)-yl)- acetamide |
| 235 | 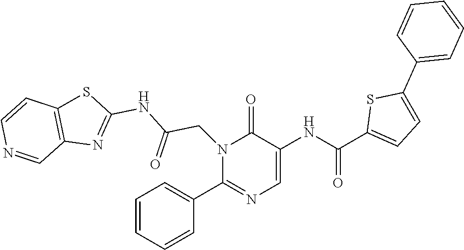 [SMILES: O=C(Cn1c(-c2ccccc2)ncc(NC(=O)c2ccc(-c3ccccc3)s2)c1=O)Nc1nc2cnccc2s1] | N-(6-ox0-1-(2-oxo-2-(thiazolo [4,5-c]pyridin-2-ylamino)- ethyl)-2-phenyl-1,6-dihydro- pyrimidin-5-yl)-5-phenylthio- phene-2-carboxamide |
| 236 | 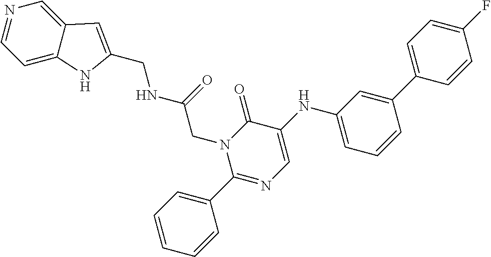 [SMILES: O=C(Cn1c(-c2ccccc2)ncc(Nc2cccc(-c3ccc(F)cc3)c2)c1=O)NCc1cc2cnccc2[nH]1] | N-((1H-pyrrolo[3,2-c]pyridin- 2-yl)methyl)-2-(5-((4′-fluoro- [1,1′-biphenyl]-3-yl)amino)-6- oxo-2-phenylpyrimidin-1(6H)- yl)acetamide |
| 237 | 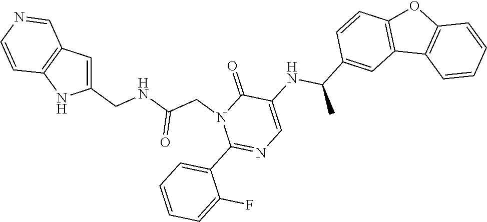 [SMILES: C[C@@H](Nc1cnc(-c2ccccc2F)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(5-((1- (dibenzo[b,d]furan-2-yl)ethyl)- amino)-2-(2-fluorophenyl)-6- oxopyrimidin-1(6H)-yl)acet- amide |
| 238 | 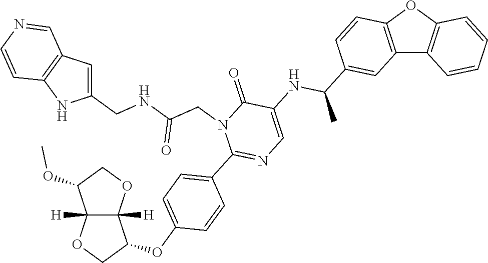 [SMILES: CO[C@@H]1CO[C@H]2[C@@H]1OC[C@H]2Oc1ccc(-c2ncc(N[C@H](C)c3ccc4oc5ccccc5c4c3)c(=O)n2CC(=O)NCc2cc3cnccc3[nH]2)cc1] | N-((1H-pyrrolo[3,2-c]pyridin- 2-yl)methyl)-2-(5-(((R)-1- (dibenzo[b,d]furan-2-yl)ethyl)- amino)-2-(4-(((3R,3aR,6R, 6aR)-6-methoxyhexahydrofuro [3,2-b]furan-3-yl)oxy)phenyl)- 6-oxopyrimidin-1(6H)-yl)acet- amide |
| 239 | 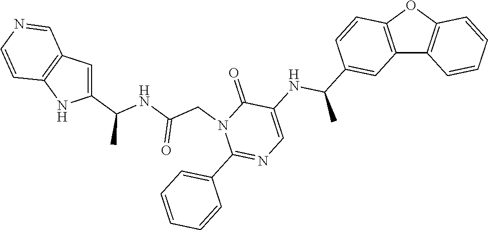 [SMILES: C[C@H](NC(=O)Cn1c(-c2ccccc2)ncc(N[C@H](C)c2ccc3oc4ccccc4c3c2)c1=O)c1cc2cnccc2[nH]1] | N-((S)-1-(1H-pyrrolo[3,2-c] pyridin-2-yl)ethyl)-2-(5-(((R)- 1-(dibenzo[b,d]furan-2-yl)- ethyl)amino)-6-oxo-2-phenyl- pyrimidin-1(6H)-yl)acetamide |
| 240 | 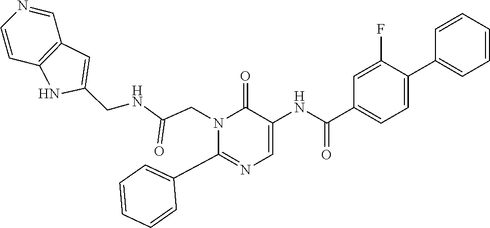 [SMILES: O=C(Cn1c(-c2ccccc2)ncc(NC(=O)c2ccc(-c3ccccc3)c(F)c2)c1=O)NCc1cc2cnccc2[nH]1] | N-(1-(2-(((1H-pyrrolo[3,2-c] pyridin-2-yl)methyl)amino)- 2-oxoethyl)-6-oxo-2-phenyl- 1,6-dihydropyrimidin-5-yl)- 2-fluoro-[1,1′-biphenyl]-4- carboxamide |
| 241 | 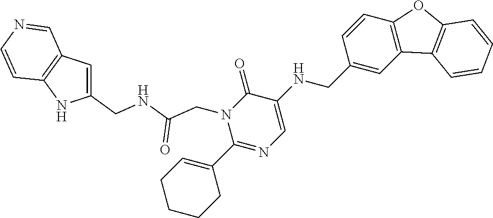 [SMILES: O=C(Cn1c(C2=CCCCC2)ncc(NCc2ccc3oc4ccccc4c3c2)c1=O)NCc1cc2cnccc2[nH]1] | N-((1H-pyrrolo[3,2-c]pyridin- 2-yl)methyl)-2-(2-(cyclohex- 1-en-1-yl)-5-((dibenzo[b,d] furan-2-ylmethyl)amino)-6- oxopyrimidin-1(6H)-yl)acet- amide |
| 242 | 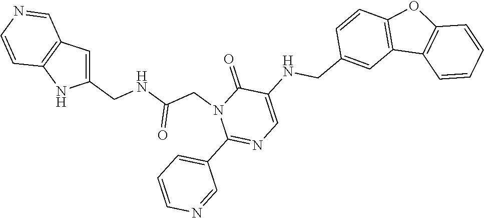 [SMILES: O=C(Cn1c(-c2cccnc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(pyridin-3-yl)pyrimi- din-1(6H)-yl)acetamide |
| 243 | 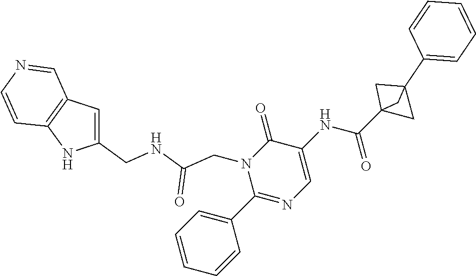 [SMILES: O=C(Cn1c(-c2ccccc2)ncc(NC(=O)C23CC(c4ccccc4)(C2)C3)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-3- phenylbicyclo[1.1.1]pentane- 1-carboxamide |
| 244 | 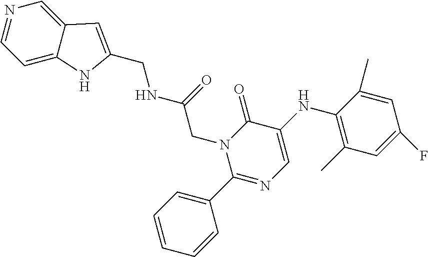 [SMILES: Cc1cc(F)cc(C)c1Nc1cnc(-c2ccccc2)n(CC(=O)NCc2cc3cnccc3[nH]2)c1=O] | N-((1H-pyrrolo[3,2-c]pyridin- 2-yl)methyl)-2-(5-((4-fluoro- 2,6-dimethylphenyl)amino)-6- oxo-2-phenylpyrimidin-1(6H)- yl)acetamide |
| 245 | 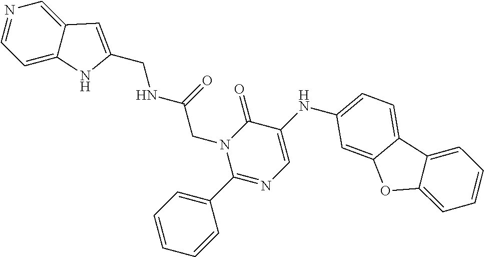 [SMILES: O=C(Cn1c(-c2ccccc2)ncc(Nc2ccc3c(c2)oc2ccccc23)c1=O)NCc1cc2cnccc2[nH]1] | N-((1H-pyrrolo[3,2-c]pyridin- 2-yl)methyl)-2-(5-dibenzo[b,d] furan-3-ylamino)-6-oxo-2- phenylpyrimidin-1(6H)-yl)- acetamide |
| 246 | 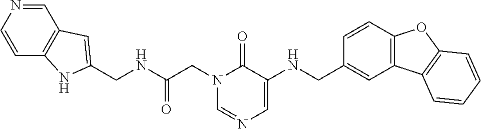 [SMILES: O=C(Cn1cncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxopyrimidin-1(6H)-yl)acet- amide |
| 247 | 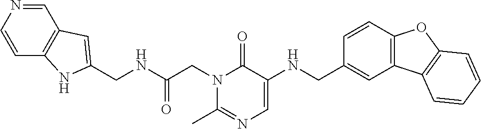 [SMILES: Cc1ncc(NCc2ccc3oc4ccccc4c3c2)c(=O)n1CC(=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-methyl-6-oxopyrimidin- 1(6H)-yl)acetamide |
| 248 | 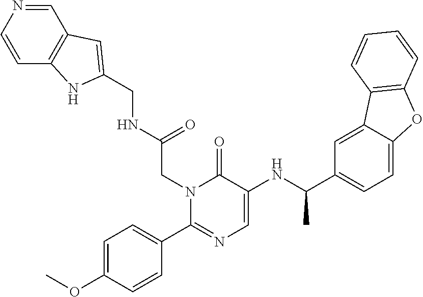 [SMILES: COc1ccc(-c2ncc(N[C@H](C)c3ccc4oc5ccccc5c4c3)c(=O)n2CC(=O)NCc2cc3cnccc3[nH]2)cc1] | (R)-N-((1H-pyrrolo[3,2-c]pyr- idin-2-yl)methyl)-2-(5-((1- (dibenzo[b,d]furan-2-yl)ethyl)- amino)-2-(4-methoxyphenyl)- 6-oxopyrimidin-1(6H)-yl)- acetamide |
| 249 | 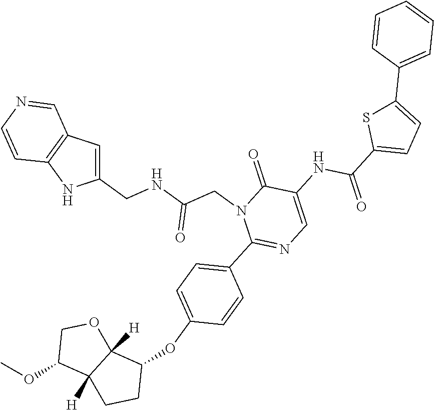 [SMILES: CO[C@@H]1CO[C@H]2[C@@H]1CC[C@H]2Oc1ccc(-c2ncc(NC(=O)c3ccc(-c4ccccc4)s3)c(=O)n2CC(=O)NCc2cc3cnccc3[nH]2)cc1] | N-(1-(2-(((1H-pyrrolo[3,2-c] pyridin-2-yl)methyl)amino)-2- oxoethyl)-2-(4-(((3R,3aR,6R, 6aR)-6-methoxyhexahydrofuro [3,2-b]furan-3-yl)oxy)phenyl)- 6-oxo-1,6-dihydropyrimidin-5- yl)-5-phenylthiophene-2- carboxamide |
| 250 | 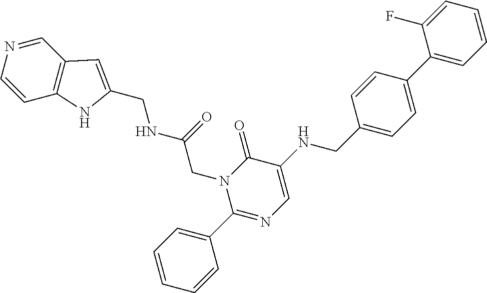 [SMILES: O=C(Cn1c(-c2ccccc2)ncc(NCc2ccc(-c3ccccc3F)cc2)c1=O)NCc1cc2cnccc2[nH]1] | N-((1H-pyrrolo[3,2-c]pyridin- 2-yl)methyl)-2-(5-(((2′-fluoro- [1,1′-biphenyl]-4-yl)methyl)- amino)-6-oxo-2-phenylpyrimi- din-1(6H)-yl)acetamide |
| 251 | 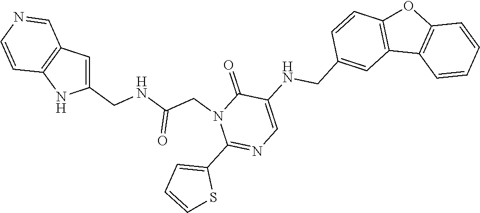 [SMILES: O=C(Cn1c(-c2cccs2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(thiophen-2-yl)pyrimi- din-1(6H)-yl)acetamide |
| 252 | 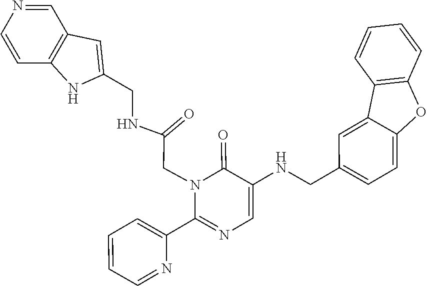 [SMILES: O=C(Cn1c(-c2ccccn2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(pyridin-2-yl)pyrimi- din-1(6H)-yl)acetamide |
| 253 | 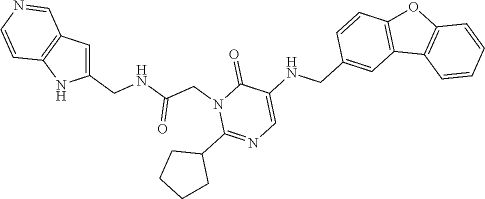 [SMILES: O=C(Cn1c(C2CCCC2)ncc(NCc2ccc3oc4ccccc4c3c2)c1=O)NCc1cc2cnccc2[nH]1] | N-((1H-pyrrolo[3,2-c]pyridin- 2-yl)methyl)-2-(2-cyclopentyl- 5-((dibenzo[b,d]furan-2-yl- methyl)amino)-6-oxopyrimi- din-1(6H)-yl)acetamide |
| 254 | 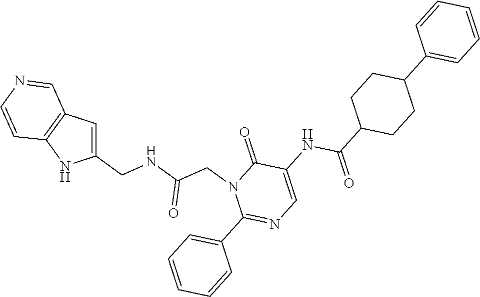 [SMILES: O=C(Cn1c(-c2ccccc2)ncc(NC(=O)C2CCC(c3ccccc3)CC2)c1=O)NCc1cc2cnccc2[nH]1] | N-(1-(2-(((1H-pyrrolo[3,2-c] pyridin-2-yl)methyl)amino)-2- oxoethyl)-6-oxo-2-phenyl-1,6- dihydropyrimidin-5-yl)-4-phen- ylcyclohexane-1-carboxamide |
| 255 | 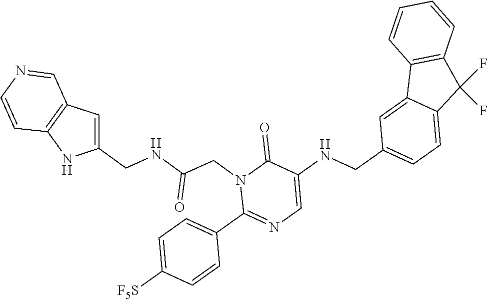 | N-((1H-pyrrolo[3,2-c]pyridin- 2-yl)methyl)-2-(5-(((9,9- difluoro-9H-fluoren-3-yl)meth- yl)amino)-6-oxo-2-(4-(penta- fluoro-lambda6-sulfaneyl)- phenyl)pyrimidin-1(6H)-yl)- acetamide |
| 256 | 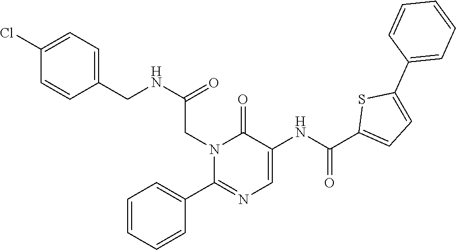 [SMILES: O=C(Cn1c(-c2ccccc2)ncc(NC(=O)c2ccc(-c3ccccc3)s2)c1=O)NCc1ccc(Cl)cc1] | N-(1-(2-((4-chlorobenzyl)- amino)-2-oxoethyl)-6-oxo-2- phenyl-1,6-dihydropyrimidin- 5-yl)-5-phenylthiophene-2- carboxamide |
| 257 | 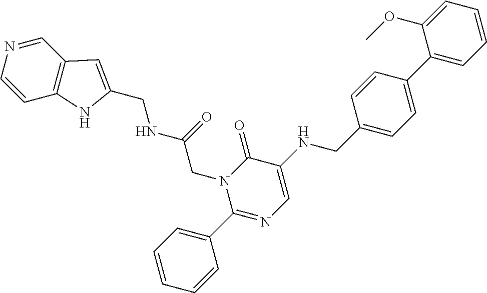 [SMILES: COc1ccccc1-c1ccc(CNc2cnc(-c3ccccc3)n(CC(=O)NCc3cc4cnccc4[nH]3)c2=O)cc1] | N-((1H-pyrrolo[3,2-c]pyridin- 2-yl)methyl)-2-(5-(((2′-meth- oxy-[1,1′-biphenyl]-4-yl)meth- yl)amino)-6-oxo-2-phenylpyr- imidin-1(6H)-yl)acetamide |
| 258 | 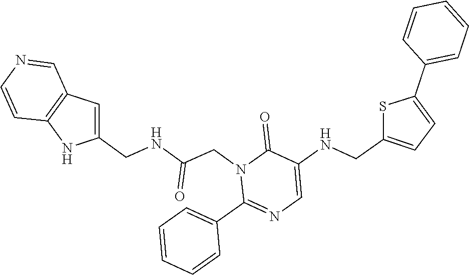 [SMILES: O=C(Cn1c(-c2ccccc2)ncc(NCc2ccc(-c3ccccc3)s2)c1=O)NCc1cc2cnccc2[nH]1] | N-((1H-pyrrolo[3,2-c]pyridin- 2-yl)methyl)-2-(6-oxo-2-phen- yl-5-(((5-phenylthiophen-2- yl)methyl)amino)pyrimidin- 1(6H)-yl)acetamide |
| 259 | 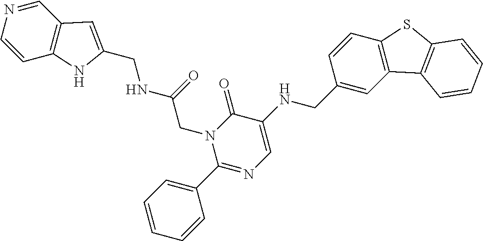 [SMILES: O=C(Cn1c(-c2ccccc2)ncc(NCc2ccc3sc4ccccc4c3c2)c1=O)NCc1cc2cnccc2[nH]1] | N-((1H-pyrrolo[3,2-c]pyridin- 2-yl)methyl)-2-(5-((dibenzo [b,d]thiophen-2-ylmethyl)- amino)-6-oxo-2-phenylpyrimi- din-1(6H)-yl)acetamide |
| 260 | 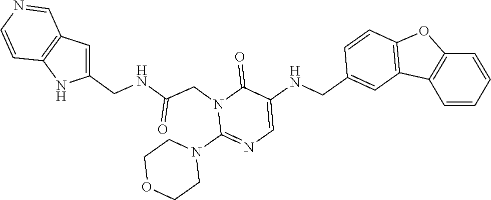 [SMILES: O=C(Cn1c(N2CCOCC2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-morpholino-6-oxopyrimidin- 1(6H)-yl)acetamide |
| 261 | 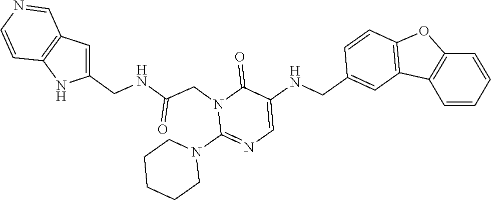 [SMILES: O=C(Cn1c(N2CCCCC2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(piperidin-1-yl)pyrimi- din-1(6H)-yl)acetamide |
| 262 | 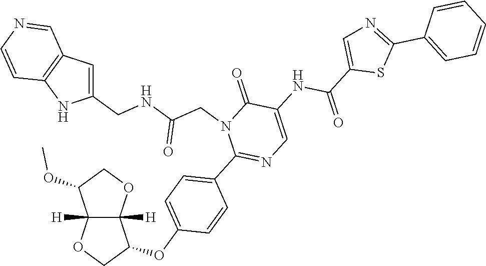 [SMILES: CO[C@@H]1CO[C@H]2[C@@H]1OC[C@H]2Oc1ccc(-c2ncc(NC(=O)c3cnc(-c4ccccc4)s3)c(=O)n2CC(=O)NCc2cc3cnccc3[nH]2)cc1] | N-(1-(2-(((1H-pyrrolo[3,2-c] pyridin-2-yl)methyl)amino)-2- oxoethyl)-2-(4-(((3R,3aR,6R, 6aR)-6-methoxyhexahydrofuro [3,2-b]furan-3-yl)oxy)phenyl)- 6-oxo-1,6-dihydropyrimidin-5- yl)-2-phenylthiazole-5-carbox- amide |
| 263 | 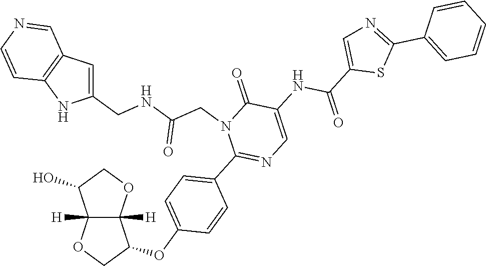 [SMILES: O=C(Cn1c(-c2ccc(O[C@@H]3CO[C@H]4[C@@H]3OC[C@H]4O)cc2)ncc(NC(=O)c2cnc(-c3ccccc3)s2)c1=O)NCc1cc2cnccc2[nH]1] | N-(1-(2-(((1H-pyrrolo[3,2-c] pyridin-2-yl)methyl)amino)-2- oxoethyl)-2-(4-(((3R,3aR,6R, 6aR)-6-hydroxyhexahydrofuro [3,2-b]furan-3-yl)oxy)phenyl)- 6-oxo-1,6-dihydropyrimidin-5- yl)-2-phenylthiazole-5-carbox- amide |
| 264 | 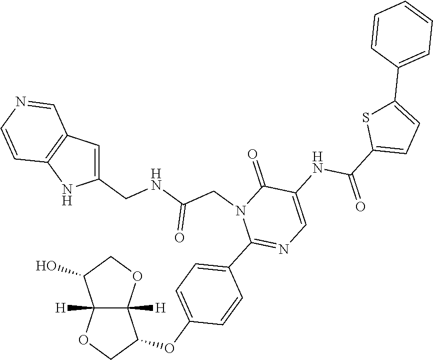 [SMILES: O=C(Cn1c(-c2ccc(O[C@@H]3CO[C@H]4[C@@H]3OC[C@H]4O)cc2)ncc(NC(=O)c2ccc(-c3ccccc3)s2)c1=O)NCc1cc2cnccc2[nH]1] | N-(1-(2-(((1H-pyrrolo[3,2-c] pyridin-2-yl)methyl)amino)-2- oxoethyl)-2-(4-(((3R,3aR,6R, 6aR)-6-hydroxyhexahydrofuro [3,2-b]furan-3-yl)oxy)phenyl)- 6-oxo-1,6-dihydropyrimidin- 5-yl)-5-phenylthiophene-2- carboxamide |
| 265 | 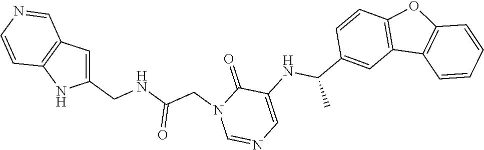 [SMILES: C[C@H](Nc1cncn(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-6-oxopyrimidin-1(6H)- yl)acetamide |
| 266 | 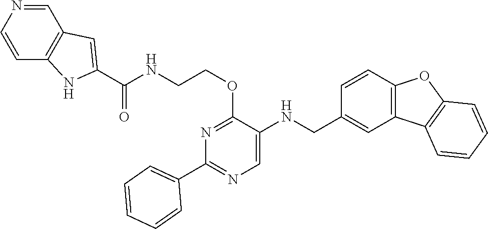 [SMILES: O=C(NCCOc1nc(-c2ccccc2)ncc1NCc1ccc2oc3ccccc3c2c1)c1cc2cnccc2[nH]1] | N-(2-((5-((dibenzo[b,d]furan- 2-ylmethyl)amino)-2-phenyl- pyrimidin-4-yl)oxy)ethyl)- 1H-pyrrolo[3,2-c]pyridine-2- carboxamide |
| 267 | 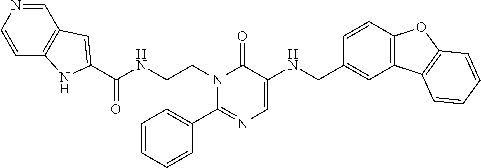 [SMILES: O=C(NCCn1c(-c2ccccc2)ncc(NCc2ccc3oc4ccccc4c3c2)c1=O)c1cc2cnccc2[nH]1] | N-(2-(5-((dibenzo[b,d]furan- 2-ylmethyl)amino)-6-oxo-2- phenylpyrimidin-1(6H)-yl)- ethyl)-1H-pyrrolo[3,2-c]pyr- idine-2-carboxamide |
| 268 | 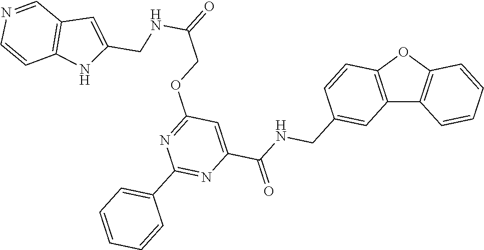 [SMILES: O=C(COc1cc(C(=O)NCc2ccc3oc4ccccc4c3c2)nc(-c2ccccc2)n1)NCc1cc2cnccc2[nH]1] | 6-(2-(((1H-pyrrolo[3,2-c]pyr- idin-2-yl)methyl)amino)-2- oxoethoxy)-N-(dibenzo[b,d] furan-2-ylmethyl)-2-phenyl- pyrimidine-4-carboxamide |
| 269 | 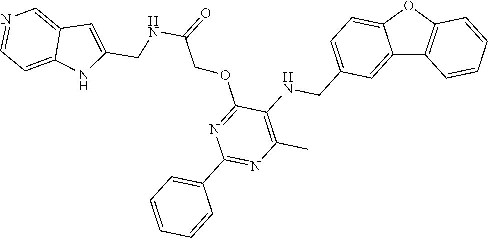 [SMILES: Cc1nc(-c2ccccc2)nc(OCC(=O)NCc2cc3cnccc3[nH]2)c1NCc1ccc2oc3ccccc3c2c1] | N-((1H-pyrrolo[3,2-c]pyridin- 2-yl)methyl)-2-((5-((dibenzo [b,d]furan-2-ylmethyl)amino)- 6-methyl-2-phenylpyrimidin-4- yl)oxy)acetamide |
| 270 | 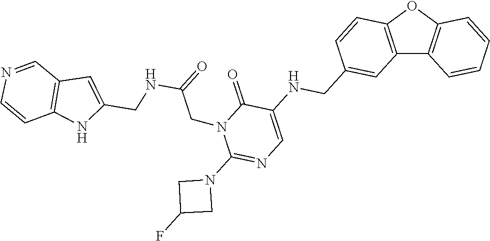 [SMILES: O=C(Cn1c(N2CC(F)C2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(3-fluoroazetidin-1-yl)-6- oxopyrimidin-1(6H)-yl)acet- amide |
| 271 | 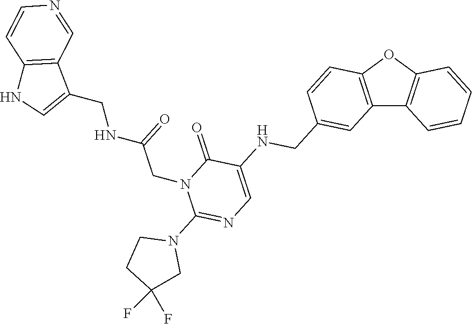 [SMILES: O=C(Cn1c(N2CCC(F)(F)C2)ncc(NCc2ccc3oc4ccccc4c3c2)c1=O)NCc1c[nH]c2ccncc12] | N-((1H-pyrrolo[3,2-c]pyridin- 3-yl)methyl)-2-(5-((dibenzo [b,d]furan-2-ylmethyl)amino)- 2-(3,3-difluoropyrrolidin-1-yl)- 6-oxopyrimidin-1(6H)-yl)acet- amide |
| 272 | 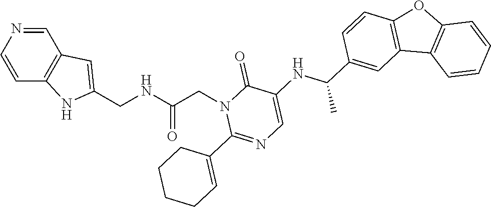 [SMILES: C[C@H](Nc1cnc(C2=CCCCC2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(2-(cyclo- hex-1-en-1-yl)-5-((1-(dibenzo [b,d]furan-2-yl)ethyl)amino)- 6-oxopyrimidin-1(6H)-yl)acet- amide |
| 273 | 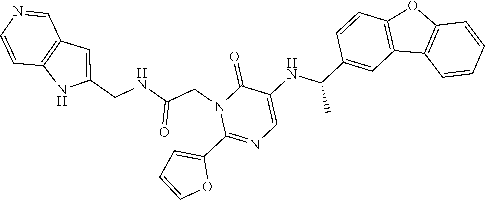 [SMILES: C[C@H](Nc1cnc(-c2ccco2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-2-(furan-2-yl)-6-oxo- pyrimidin-1(6H)-yl)acetamide |
| 274 | 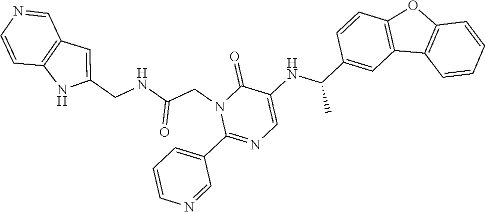 [SMILES: C[C@H](Nc1cnc(-c2cccnc2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-6-oxo-2-(pyridin-3-yl)- pyrimidin-1(6H)-yl)acetamide |
| 275 | 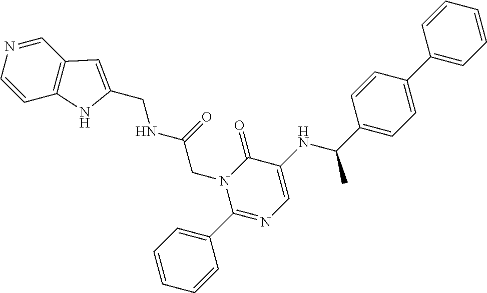 [SMILES: C[C@@H](Nc1cnc(-c2ccccc2)n(CC(=O)NCc2cc3cnccc3[nH]2)c1=O)c1ccc(-c2ccccc2)cc1] | (R)-N-((1H-pyrrolo[3,2-c]pyr- idin-2-yl)methyl)-2-(5-((1- ([1,1′-biphenyl]-4-yl)ethyl)- amino)-6-oxo-2-phenylpyrimi- din-1(6H)-yl)acetamide |
| 276 | 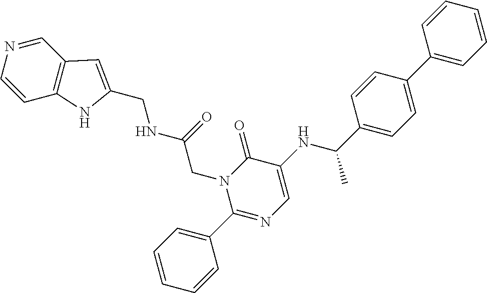 [SMILES: C[C@H](Nc1cnc(-c2ccccc2)n(CC(=O)NCc2cc3cnccc3[nH]2)c1=O)c1ccc(-c2ccccc2)cc1] | (S)-N-((1H-pyrrolo[3,2-c]pyr- idin-2-yl)methyl)-2-(5-((1- ([1,1′-biphenyl]-4-yl)ethyl)- amino)-6-oxo-2-phenylpyrimi- din-1(6H)-yl)acetamide |
| 277 | 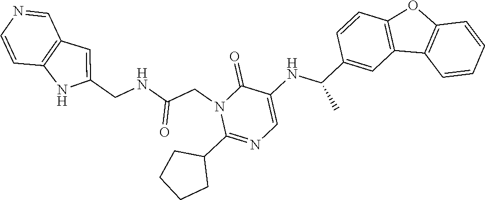 [SMILES: C[C@H](Nc1cnc(C2CCCC2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(2-cyclo- pentyl-5-((1-(dibenzo[b,d] furan-2-yl)ethyl)amino)-6-oxo- pyrimidin-1(6H)-yl)acetamide |
| 278 | 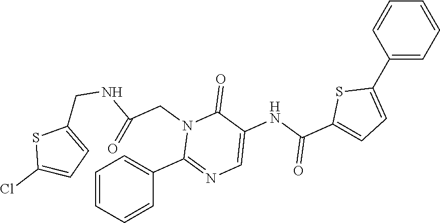 [SMILES: O=C(Cn1c(-c2ccccc2)ncc(NC(=O)c2ccc(-c3ccccc3)s2)c1=O)NCc1ccc(Cl)s1] | N-(1-(2-(((5-chlorothiophen-2- yl)methyl)amino)-2-oxoethyl)- 6-oxo-2-phenyl-1,6-dihydro- pyrimidin-5-yl)-5-phenylthio- phene-2-carboxamide |
| 279 | 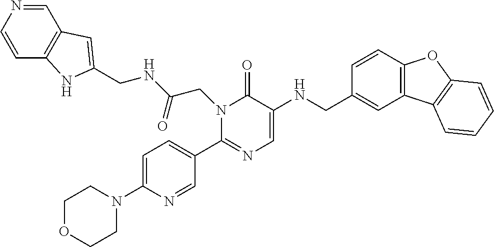 [SMILES: O=C(Cn1c(-c2ccc(N3CCOCC3)nc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(6-morpholinopyridin-3-yl)- 6-oxopyrimidin-1(6H)-yl)- acetamide |
| 280 | 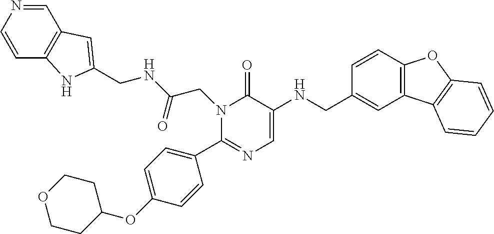 [SMILES: O=C(Cn1c(-c2ccc(OC3CCOCC3)cc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(4-((tetrahydro-2H- pyran-4-yl)oxy)phenyl)pyrimi- din-1(6H)-yl)acetamide |
| 281 | 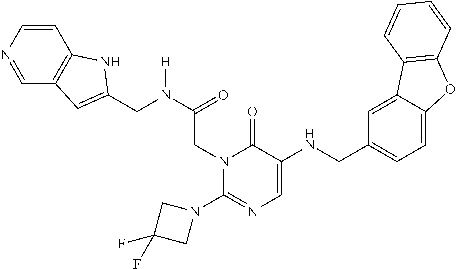 [SMILES: O=C(Cn1c(N2CC(F)(F)C2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 2-(3,3-difluoroazetidin-1-yl)-6- oxopyrimidin-1(6H)-yl)acet- amide |
| 282 | 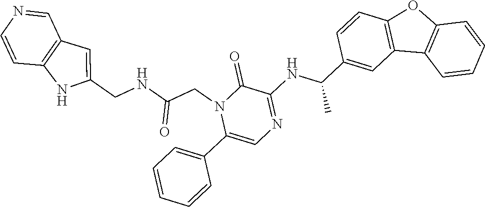 [SMILES: C[C@H](Nc1ncc(-c2ccccc2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(3-((1- (dibenzo[b,d]furan-2-yl)ethyl)- amino)-2-oxo-6-phenylpyrazin- 1(2H)-yl)acetamide |
| 283 | 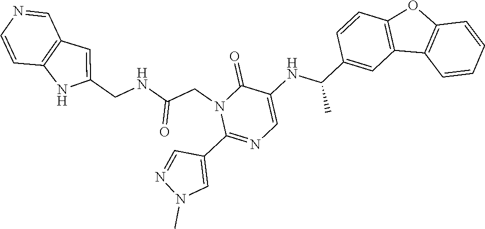 [SMILES: C[C@H](Nc1cnc(-c2cnn(C)c2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-2-(1-methyl-1H-pyr- azol-4-yl)-6-oxopyrimidin- 1(6H)-yl)acetamide |
| 284 | 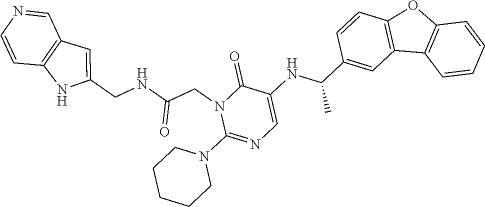 [SMILES: C[C@H](Nc1cnc(N2CCCCC2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-6-oxo-2-(piperidin-1- yl)pyrimidin-1(6H)-yl)acet- amide |
| 285 | 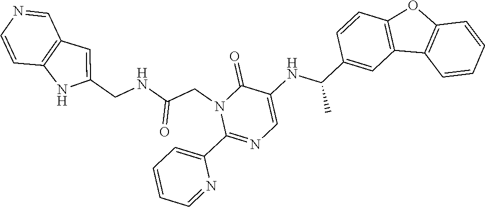 [SMILES: C[C@H](Nc1cnc(-c2ccccn2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-6-oxo-2-(pyridin-2-yl)- pyrimidin-1(6H)-yl)acetamide |
| 286 | 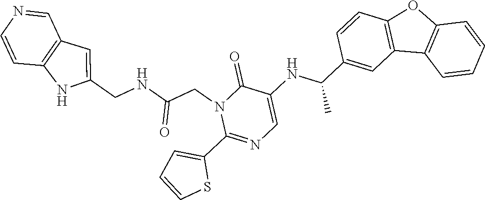 [SMILES: C[C@H](Nc1cnc(-c2cccs2)n(CC(=O)NCc2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-6-oxo-2-(thiophen-2- yl)pyrimidin-1(6H)-yl)acet- amide |
| 287 | 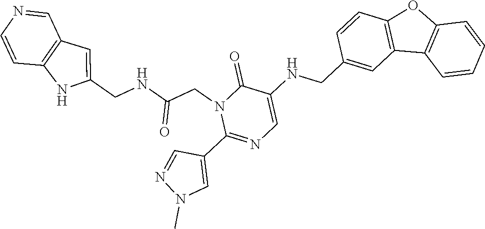 [SMILES: Cn1cc(-c2ncc(NCc3ccc4oc5ccccc5c4c3)c(=O)n2CC(=O)NCc2cc3cnccc3[nH]2)cn1] | N-((1H-pyrrolo[3,2-c]pyridin- 2-yl)methyl)-2-(5-((dibenzo [b,d]furan-2-ylmethyl)amino)- 2-(1-methyl-1H-pyrazol-4-yl)- 6-oxopyrimidin-1(6H)-yl)- acetamide |
| 288 | 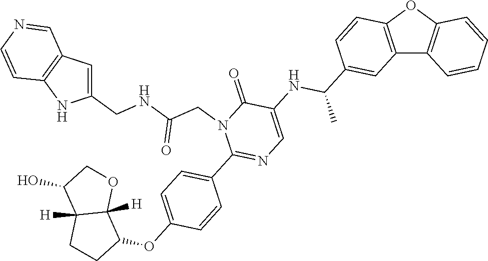 | N-((1H-pyrrolo[3,2-c]pyridin- 2-yl)methyl)-2-(5-(((S)-1- (dibenzo[b,d]furan-2-yl)ethyl)- amino)-2-(4-(((3R,3aR,6R, 6aR)-6-hydroxyhexahydrofuro [3,2-b]furan-3-yl)oxy)phenyl)- 6-oxopyrimidin-1(6H)-yl)acet- amide |
| 289 | 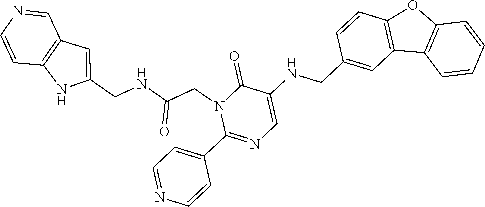 [SMILES: O=C(Cn1c(-c2ccncc2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(pyridin-4-yl)pyrimi- din-1(6H)-yl)acetamide |
| 290 | 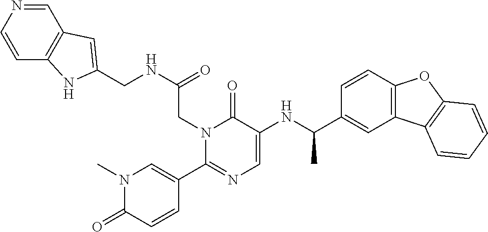 [SMILES: C[C@@H](Nc1cnc(-c2ccc(=O)n(C)c2)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(5-((1- (dibenzo[b,d]furan-2-yl)ethyl)- amino)-2-(1-methyl-6-oxo-1,6- dihydropyridin-3-yl)-6-oxopyr- imidin-1(6H)-yl)acetamide |
| 291 | 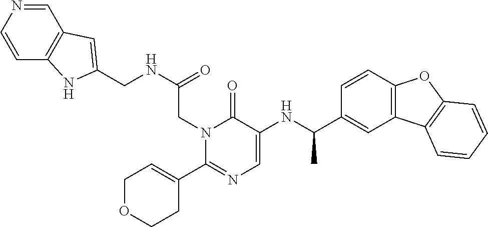 [SMILES: C[C@@H](Nc1cnc(C2=CCOCC2)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(5-((1- (dibenzo[b,d]furan-2-yl)ethyl)- amino)-2-(3,6-dihydro-2H- pyran-4-yl)-6-oxopyrimidin- 1(6H)-yl)acetamide |
| 292 | 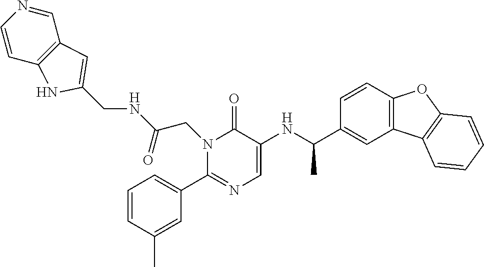 [SMILES: Cc1cccc(-c2ncc(N[C@H](C)c3ccc4oc5ccccc5c4c3)c(=O)n2CC(=O)NCc2cc3cnccc3[nH]2)c1] | (R)-N-((1H-pyrrolo[3,2-c]pyr- idin-2-yl)methyl)-2-(5-((1- (dibenzo[b,d]furan-2-yl)ethyl)- amino)-6-oxo-2-(m-tolyl)pyr- imidin-1(6H)-yl)acetamide |
| 293 | 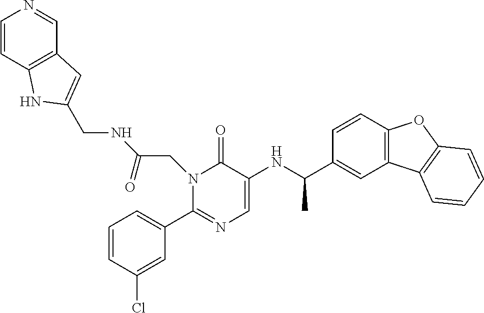 [SMILES: C[C@@H](Nc1cnc(-c2cccc(Cl)c2)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(2-(3- chlorophenyl)-5-((1-(dibenzo [b,d]furan-2-yl)ethyl)amino)- 6-oxopyrimidin-1(6H)-yl)acet- amide |
| 294 | 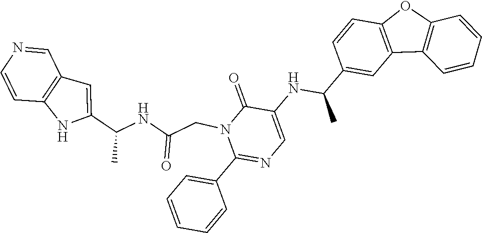 [SMILES: C[C@@H](Nc1cnc(-c2ccccc2)n(CC(=O)N[C@H](C)c2cc3cnccc3[nH]2)c1=O)c1ccc2oc3ccccc3c2c1] | N-((R)-1-(1H-pyrrolo[3,2-c] pyridin-2-yl)ethyl)-2-(5-(((R)- 1-(dibenzo[b,d]furan-2-l)ethyl)- amino)-6-oxo-2-phenylpyrimi- din-1(6H)-yl)acetamide |
| 295 | 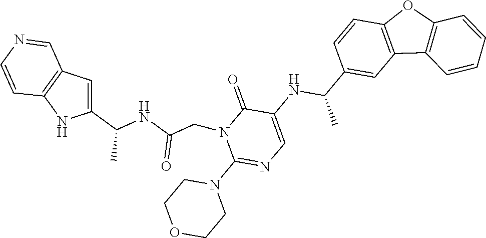 [SMILES: C[C@H](Nc1cnc(N2CCOCC2)n(CC(=O)N[C@H](C)c2cc3cnccc3[nH]2)c1=O)c1ccc2oc3ccccc3c2c1] | (S)-N-((1H-pyrrolo[3,2-c]pyr- idin-2-yl)methyl)-2-(5-((1- (dibenzo[b,d]furan-2-yl)ethyl)- amino)-2-morpholino-6-oxo- pyrimidin-1(6H)-yl)acetamide |
| 296 | 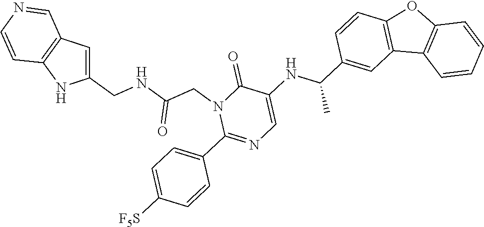 | (S)-N-((1H-pyrrolo[3,2-c] pyridin-2-yl)methyl)-2-(5-((1- (dibenzo[b,d]furan-2-yl)ethyl)- amino)-6-oxo-2-(4-(penta- fluoro-lambda6-sulfaneyl)- phenyl)pyrimidin-1(6H)-yl)- acetamide |
| 297 | 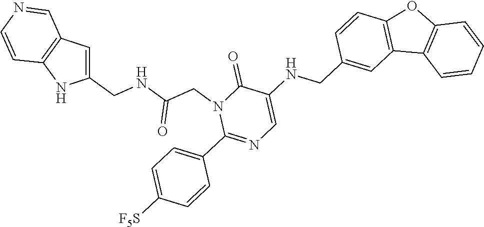 | N-((1H-pyrrolo[3,2-c]pyridin- 2-yl)methyl)-2-(5-((dibenzo [b,d]furan-2-ylmethyl)amino)- 6-oxo-2-(4-(pentafluoro- lambda6-sulfaneyl)phenyl)pyr- imidin-1(6H)-yl)acetamide |
| 298 | 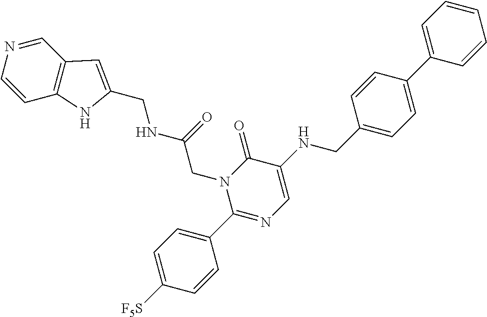 | N-((1H-pyrrolo[3,2-c]pyridin- 2-yl)methyl)-2-(5-(([1,1′- biphenyl]-4-ylmethyl)amino)- 6-oxo-2-(4-(pentafluoro- lambda6-sulfaneyl)phenyl)pyr- imidin-1(6H)-yl)acetamide |
| 299 | 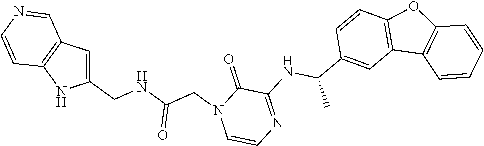 [SMILES: C[C@H](Nc1nccn(CC(=O)NCc2cc3cnccc3[nH]2)c1=O)c1ccc2oc3ccccc3c2c1] | (S)-N-((1H-pyrrolo[3,2-c] pyridin-2-yl)methyl)-2-(3-((1- (dibenzo[b,d]furan-2-yl)ethyl)- amino)-2-oxopyrazin-1(2H)- yl)acetamide |
| 300 | 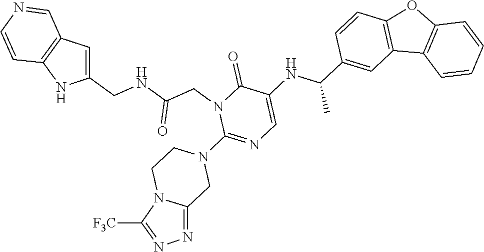 [SMILES: C[C@H](Nc1cnc(N2CCn3c(nnc3C(F)(F)F)C2)n(CC(=O)NCc2cc3cnccc3[nH]2)c1=O)c1ccc2oc3ccccc3c2c1] | (S)-N-((1H-pyrrolo[3,2-c] pyridin-2-yl)methyl)-2-(5-((1- (dibenzo[b,d]furan-2-yl)ethyl)- amino)-6-oxo-2-(3-(trifluoro- methyl)-5,6-dihydro-[1,2,4] triazolo[4,3-a]pyrazin-7(8H)- yl)pyrimidin-1(6H)-yl)acet- amide |
| 301 | 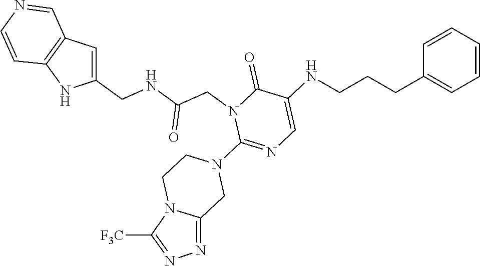 [SMILES: O=C(Cn1c(N2CCn3c(nnc3C(F)(F)F)C2)ncc(NCCCc2ccccc2)c1=O)NCc1cc2cnccc2[nH]1] | N-((1H-pyrrolo[3,2-c]pyridin- 2-yl)methyl)-2-(6-oxo-5-((3- phenylpropyl)amino)-2-(3- (trifluoromethyl)-5,6-dihydro- [1,2,4]triazolo[4,3-a]pyrazin- 7(8H)-yl)pyrimidin-1(6H)-yl)- acetamide |
| 302 | 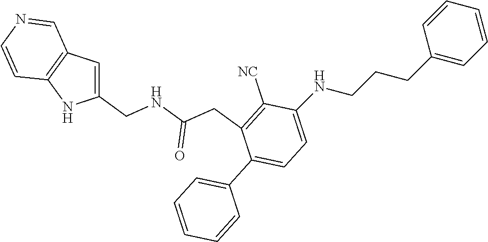 [SMILES: N#Cc1c(NCCCc2ccccc2)ccc(-c2ccccc2)c1CC(=O)NCc1cc2cnccc2[nH]1] | N-((1H-pyrrolo[3,2-c]pyridin- 2-yl)methyl)-2-(3-cyano-4-((3- phenylpropyl)amino)-[1,1′- biphenyl]-2-yl)acetamide |
| 303 | 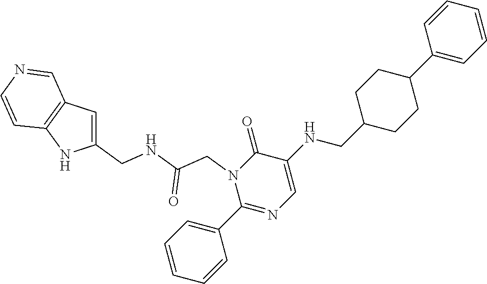 [SMILES: O=C(Cn1c(-c2ccccc2)ncc(NCC2CCC(c3ccccc3)CC2)c1=O)NCc1cc2cnccc2[nH]1] | N-((1H-pyrrolo[3,2-c]pyridin- 2-yl)methyl)-2-(6-oxo-2-phen- yl-5-(((4-phenylcyclohexyl)- methyl)amino)pyrimidin- 1(6H)-yl)acetamide |
| 304 | 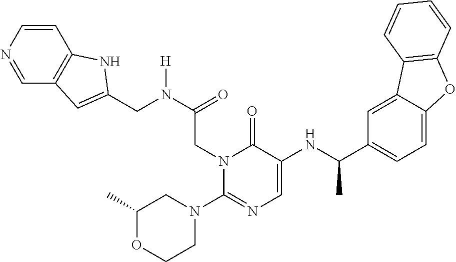 [SMILES: C[C@@H]1CN(c2ncc(N[C@H](C)c3ccc4oc5ccccc5c4c3)c(=O)n2CC(=O)NCc2cc3cnccc3[nH]2)CCO1] | N-((1H-pyrrolo[3,2-c]pyridin- 2-yl)methyl)-2-(5-(((R)-1- (dibenzo[b,d]furan-2-yl)ethyl)- amino)-2-((R)-2-methylmorph- olino)-6-oxopyrimidin-1(6H)- yl)acetamide |
| 305 | 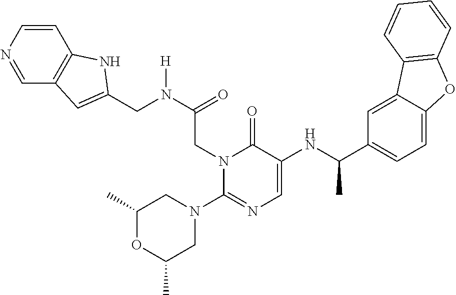 [SMILES: C[C@@H]1CN(c2ncc(N[C@H](C)c3ccc4oc5ccccc5c4c3)c(=O)n2CC(=O)NCc2cc3cnccc3[nH]2)C[C@H](C)O1] | N-((1H-pyrrolo[3,2-c]pyridin- 2-yl)methyl)-2-(5-(((R)-1- (dibenzo[b,d]furan-2-yl)ethyl)- amino)-2-((2S,6R)-2,6-dimeth- ylmorpholino)-6-oxopyrimidin- 1(6H)-yl)acetamide |
| 306 | 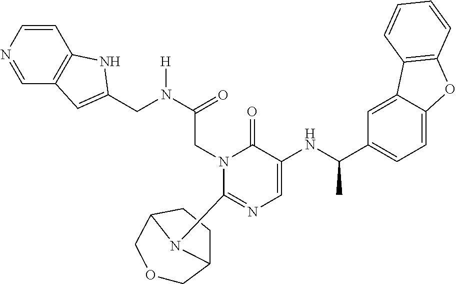 [SMILES: C[C@@H](Nc1cnc(N2C3CCC2COC3)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(2-(3-oxa-8- azabicyclo[3.2.1]octan-8-yl)-5- (((R)-1-(dibenzo[b,d]furan-2- yl)ethyl)amino)-6-oxopyrimi- din-1(6H)-yl)acetamide |
| 307 | 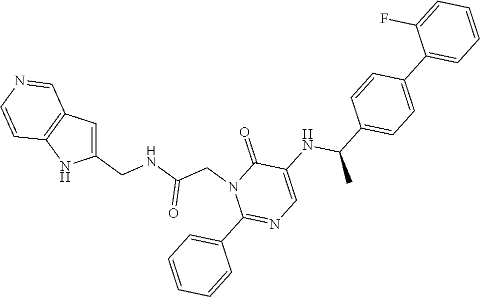 [SMILES: C[C@@H](Nc1cnc(-c2ccccc2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(5-((1- (2′-fluoro-[1,1′-biphenyl]-4- yl)ethyl)amino)-6-oxo-2-phen- ylpyrimidin-1(6H)-yl)acet- amide |
| 308 | 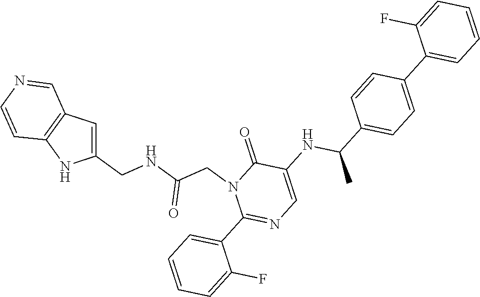 [SMILES: C[C@@H](Nc1cnc(-c2ccccc2F)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(5-((1- (2′-fluoro-[1,1′-biphenyl]-4-yl)- ethyl)amino)-2-(2-fluorophen- yl)-6-oxopyrimidin-1(6H)-yl)- acetamide |
| 309 | 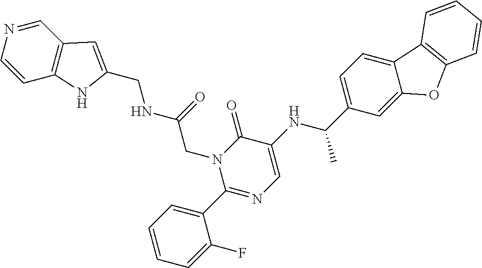 [SMILES: C[C@H](Nc1cnc(-c2ccccc2F)n(CC(=O)NCc2cc3cnccc3[nH]2)c1=O)c1ccc2c(c1)oc1ccccc12] | (S)-N-((1H-pyrrolo[3,2-c] pyridin-2-yl)methyl)-2-(5-((1- (dibenzo[b,d]furan-3-yl)ethyl)- amino)-2-(2-fluorophenyl)-6- oxopyrimidin-1(6H)-yl)acet- amide |
| 310 | 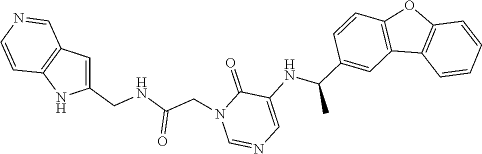 [SMILES: C[C@@H](Nc1cncn(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxopyrimidin-1(6H)- yl)acetamide |
| 311 | 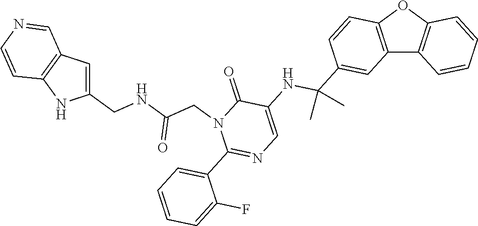 [SMILES: CC(C)(Nc1cnc(-c2ccccc2F)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(5-((2-(dibenzo [b,d]furan-2-yl)propan-2-yl)- amino)-2-(2-fluorophenyl)-6- oxopyrimidin-1(6H)-yl)acet- amide |
| 312 | 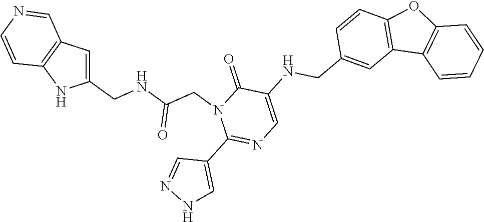 [SMILES: O=C(Cn1c(-c2cn[nH]c2)ncc(NCc2ccc3oc4ccccc4c3c2)c1=O)NCc1cc2cnccc2[nH]1] | N-((1H-pyrrolo[3,2-c]pyridin- 2-yl)methyl)-2-(5-((dibenzo [b,d]furan-2-ylmethyl)amino)- 6-oxo-2-(1H-pyrazol-4-yl)pyr- imidin-1(6H)-yl)acetamide |
| 313 | 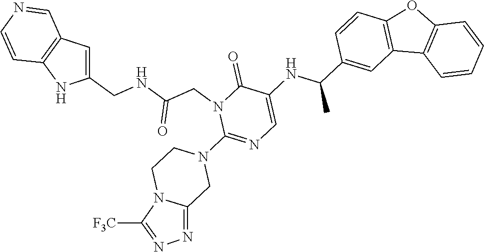 [SMILES: C[C@@H](Nc1cnc(N2CCn3c(nnc3C(F)(F)F)C2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(3-(trifluoro- methyl)-5,6-dihydro-[1,2,4] triazolo[4,3-a]pyrazin-7(8H)- yl)pyrimidin-1(6H)-yl)acet- amide |
| 314 | 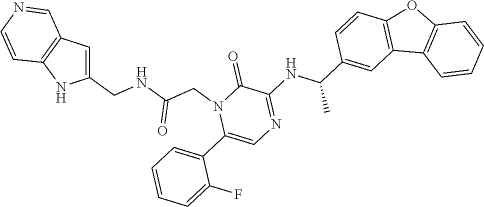 [SMILES: C[C@H](Nc1ncc(-c2ccccc2F)n(CC(=O)NCc2cc3cnccc3[nH]2)c1=O)c1ccc2oc3ccccc3c2c1] | (S)-N-((1H-pyrrolo[3,2-c] pyridin-2-yl)methyl)-2-(3-((1- (dibenzo[b,d]furan-2-yl)ethyl)- amino)-6-(2-fluorophenyl)-2- oxopyrazin-1(2H)-yl)acetamide |
| 315 | 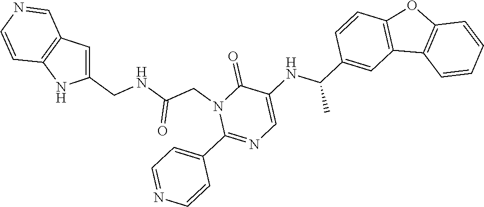 [SMILES: C[C@H](Nc1cnc(-c2ccncc2)n(CC(=O)NCc2cc3cnccc3[nH]2)c1=O)c1ccc2oc3ccccc3c2c1] | (S)-N-((1H-pyrrolo[3,2-c] pyridin-2-yl)methyl)-2-(5-((1- (dibenzo[b,d]furan-2-yl)ethyl)- amino)-6-oxo-2-(pyridin-4-yl)- pyrimidin-1(6H)-yl)acetamide |
| 316 | 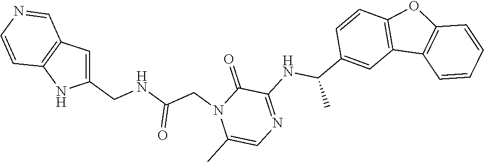 [SMILES: Cc1cnc(N[C@@H](C)c2ccc3oc4ccccc4c3c2)c(=O)n1CC(=O)NCc1cc2cnccc2[nH]1] | (S)-N-((1H-pyrrolo[3,2-c] pyridin-2-yl)methyl)-2-(3-((1- (dibenzo[b,d]furan-2-yl)ethyl)- amino)-6-methyl-2-oxopyrazin- 1(2H)-yl)acetamide |
| 317 | 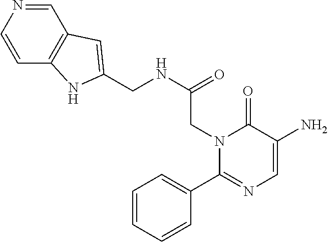 [SMILES: Nc1cnc(-c2ccccc2)n(CC(=O)NCc2cc3cnccc3[nH]2)c1=O] | N-((1H-pyrrolo[3,2-c]pyridin- 2-yl)methyl)-2-(5-amino-6-oxo- 2-phenylpyrimidin-1(6H)-yl)- acetamide |
| 318 | 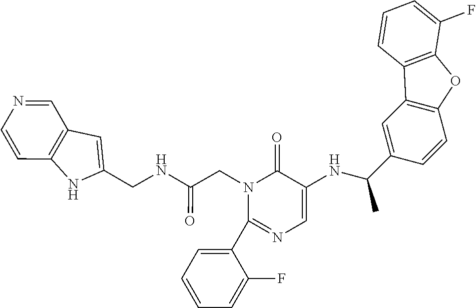 [SMILES: C[C@@H](Nc1cnc(-c2ccccc2F)n(CC(=O)NCc2cc3cnccc3[nH]2)c1=O)c1ccc2oc3c(F)cccc3c2c1] | (R)-N-((1H-pyrrolo[3,2-c]pyr- idin-2-yl)methyl)-2-(5-((1-(6- fluorodibenzo[b,d]furan-2-yl)- ethyl)amino)-2-(2-fluorophen- yl)-6-oxopyrimidin-1(6H)-yl)- acetamide |
| 319 | 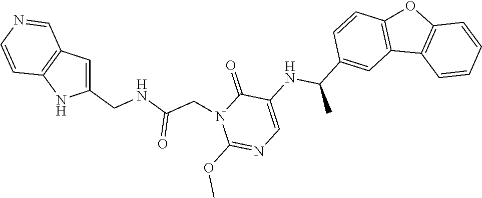 [SMILES: COc1ncc(N[C@H](C)c2ccc3oc4ccccc4c3c2)c(=O)n1CC(=O)NCc1cc2cnccc2[nH]1] | (R)-N-((1H-pyrrolo[3,2-c] pyridin-2-yl)methyl)-2-(5-((1- (dibenzo[b,d]furan-2-yl)ethyl)- amino)-2-methoxy-6-oxopyr- imidin-1(6H)-yl)acetamide |
| 320 | 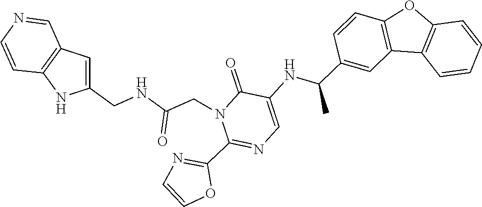 [SMILES: C[C@@H](Nc1cnc(-c2ncco2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(oxazol-2-yl)-6-oxo- pyrimidin-1(6H)-yl)acetamide |
| 321 | 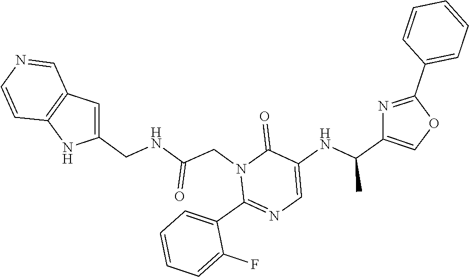 [SMILES: C[C@@H](Nc1cnc(-c2ccccc2F)n(CC(=O)NCc2cc3cnccc3[nH]2)c1=O)c1coc(-c2ccccc2)n1] | (R)-N-((1H-pyrrolo[3,2-c] pyridin-2-yl)methyl)-2-(2-(2- fluorophenyl)-6-oxo-5-((1-(2- phenyloxazol-4-yl)ethyl)- amino)pyrimidin-1(6H)-yl)- acetamide |
| 322 | 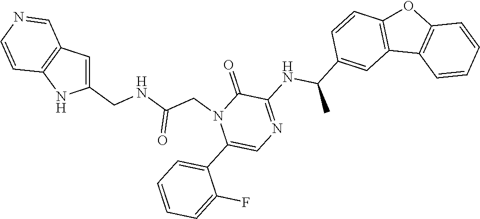 [SMILES: C[C@@H](Nc1ncc(-c2ccccc2F)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-6-(2-fluorophenyl)-2- oxopyrazin-1(2H)-yl)acetamide |
| 323 | 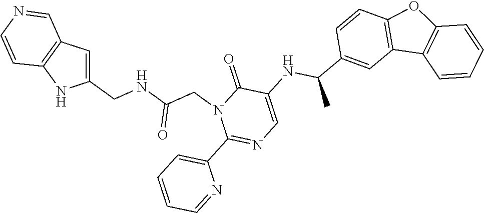 [SMILES: C[C@@H](Nc1cnc(-c2ccccn2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(pyridin-2-yl)- pyrimidin-1(6H)-yl)acetamide |
| 324 | 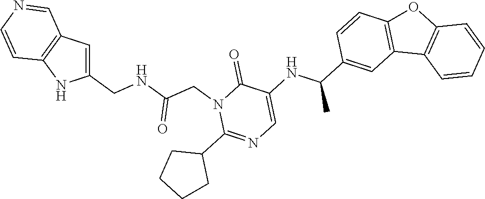 [SMILES: C[C@@H](Nc1cnc(C2CCCC2)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(2-cyclo- pentyl-5-((1-(dibenzo[b,d] furan-2-yl)ethyl)amino)-6-oxo- pyrimidin-1(6H)-yl)acetamide |
| 325 | 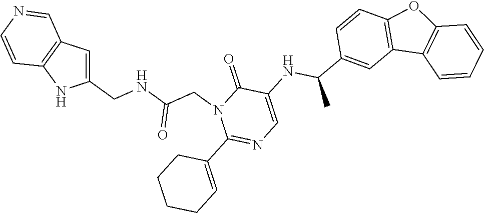 [SMILES: C[C@@H](Nc1cnc(C2=CCCCC2)n(CC(=O)NCc2cc3cnccc3[nH]2)c1=O)c1ccc2oc3ccccc3c2c1] | (R)-N-((1H-pyrrolo[3,2-c]pyr- idin-2-yl)methyl)-2-(2-(cyclo- hex-1-en-1-yl)-5-((1-(dibenzo [b,d]furan-2-yl)ethyl)amino)- 6-oxopyrimidin-1(6H)-yl)acet- amide |
| 326 | 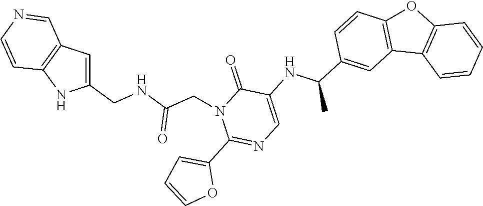 [SMILES: C[C@@H](Nc1cnc(-c2ccco2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(furan-2-yl)-6-oxo- pyrimidin-1(6H)-yl)acetamide |
| 327 | 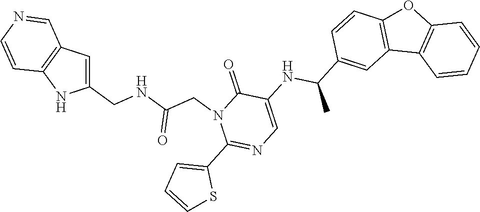 [SMILES: C[C@@H](Nc1cnc(-c2cccs2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(thiophen-2- yl)pyrimidin-1(6H)-yl)acet- amide |
| 328 | 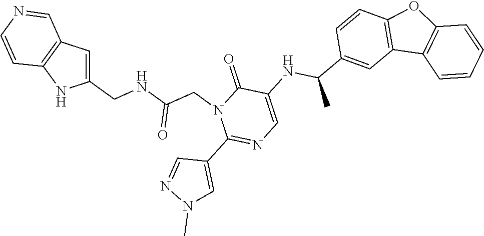 [SMILES: C[C@@H](Nc1cnc(-c2cnn(C)c2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(1-methyl-1H-pyr- azol-4-yl)-6-oxopyrimidin- 1(6H)-yl)acetamide |
| 329 | 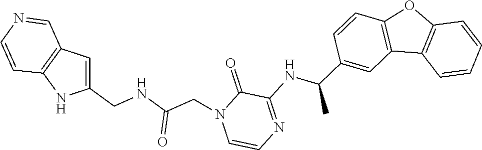 [SMILES: C[C@@H](Nc1nccn(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxopyrazin-1(2H)- yl)acetamide |
| 330 | 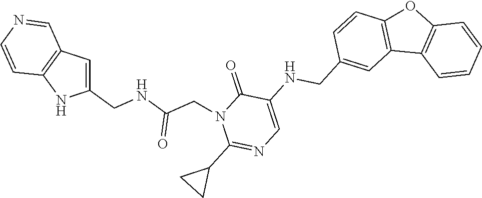 [SMILES: O=C(Cn1c(C2CC2)ncc(NCc2ccc3oc4ccccc4c3c2)c1=O)NCc1cc2cnccc2[nH]1] | N-((1H-pyrrolo[3,2-c]pyridin- 2-yl)methyl)-2-(2-cyclopropyl- 5-((dibenzo[b,d]furan-2-yl- methyl)amino)-6-oxopyrimi- din-1(6H)-yl)acetamide |
| 331 | 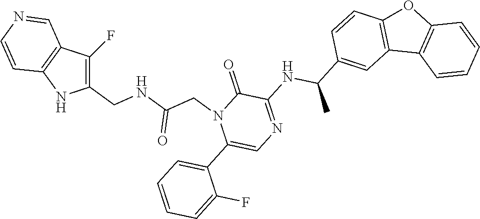 [SMILES: C[C@@H](Nc1ncc(-c2ccccc2F)n(CC(=O)NCc2[nH]c3ccncc3c2F)c1=O)c1ccc2oc3ccccc3c2c1] | (R)-2-(3-((1-(dibenzo[b,d] furan-2-yl)ethyl)amino)-6-(2- fluorophenyl)-2-oxopyrazin- 1(2H)-yl)-N-((3-fluoro-1H- pyrrolo[3,2-c]pyridin-2-yl)- methyl)acetamide |
| 332 | 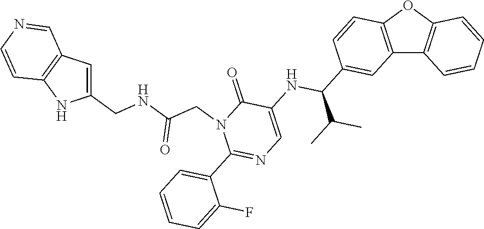 [SMILES: CC(C)[C@@H](Nc1cnc(-c2ccccc2F)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)-2- methylpropyl)amino)-2-(2- fluorophenyl)-6-oxopyrimidin- 1(6H)-yl)acetamide |
| 333 | 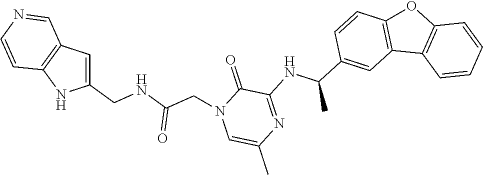 [SMILES: Cc1cn(CC(=O)NCc2cc3cnccc3[nH]2)c(=O)c(N[C@H](C)c2ccc3oc4ccccc4c3c2)n1] | (R)-N-((1H-pyrrolo[3,2-c] pyridin-2-yl)methyl)-2-(3-((1- (dibenzo[b,d]furan-2-yl)ethyl)- amino)-5-methyl-2-oxopyrazin- 1(2H)-yl)acetamide |
| 334 | 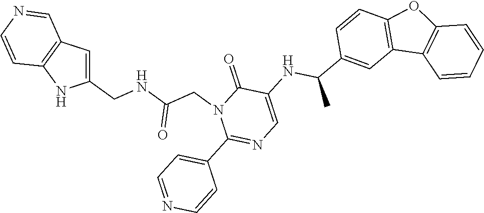 [SMILES: C[C@@H](Nc1cnc(-c2ccncc2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(pyridin-4-yl)- pyrimidin-1(6H)-yl)acetamide |
| 335 | 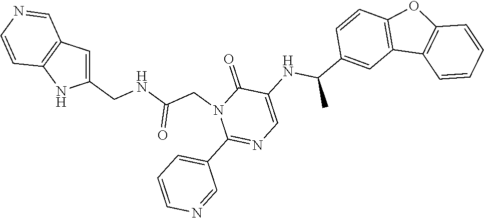 [SMILES: C[C@@H](Nc1cnc(-c2cccnc2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(pyridin-3-yl)- pyrimidin-1(6H)-yl)acetamide |
| 336 | 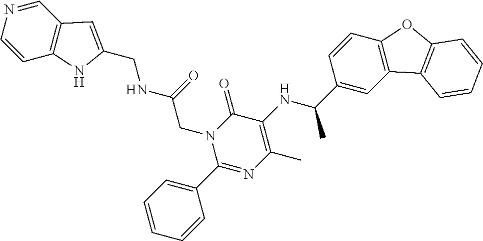 [SMILES: Cc1nc(-c2ccccc2)n(CC(=O)NCc2cc3cnccc3[nH]2)c(=O)c1N[C@H](C)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-4-methyl-6-oxo-2-phen- ylpyrimidin-1(6H)-yl)-acet- amide |
| 337 | 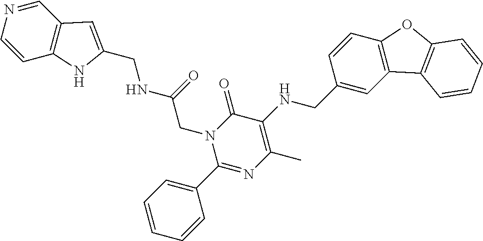 [SMILES: Cc1nc(-c2ccccc2)n(CC(=O)NCc2cc3cnccc3[nH]2)c(=O)c1NCc1ccc2oc3ccccc3c2c1] | N-((1H-pyrrolo[3,2-c]pyridin- 2-yl)methyl)-2-(5-((dibenzo [b,d]furan-2-ylmethyl)amino)- 4-methyl-6-oxo-2-phenylpyr- imidin-1(6H)-yl)acetamide |
| 338 | 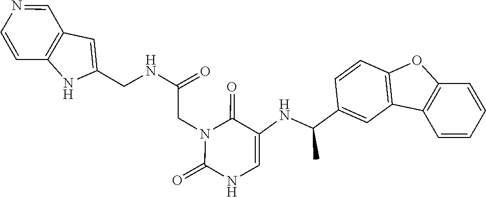 [SMILES: C[C@@H](Nc1c[nH]c(=O)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2,6-dioxo-3,6-dihydro- pyrimidin-1(2H)-yl)acetamide |
| 339 | 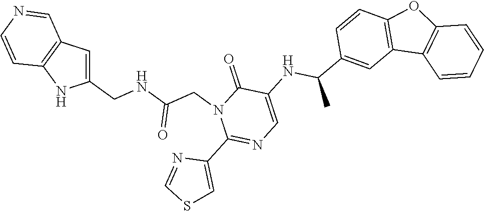 [SMILES: C[C@@H](Nc1cnc(-c2cscn2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(thiazol-4-yl)- pyrimidin-1(6H)-yl)acetamide |
| 340 | 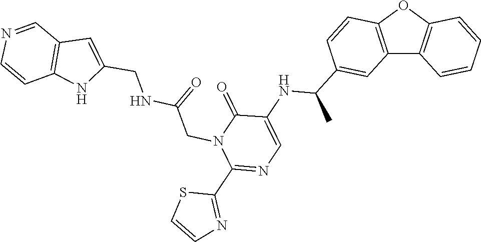 [SMILES: C[C@@H](Nc1cnc(-c2nccs2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(thiazol-2-yl)- pyrimidin-1(6H)-yl)acetamide |
| 341 | 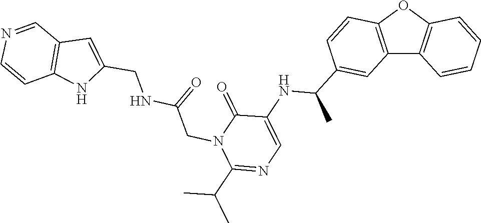 [SMILES: CC(C)c1ncc(N[C@H](C)c2ccc3oc4ccccc4c3c2)c(=O)n1CC(=O)NCc1cc2cnccc2[nH]1] | (R)-N-((1H-pyrrolo[3,2-c] pyridin-2-yl)methyl)-2-(5-((1- (dibenzo[b,d]furan-2-yl)ethyl)- amino)-2-isopropyl-6-oxopyr- imidin-1(6H)-yl)acetamide |
| 342 | 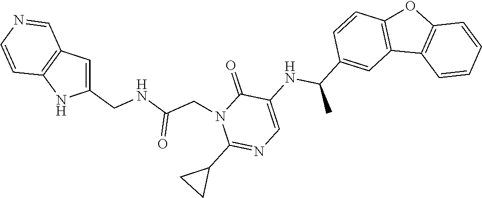 [SMILES: C[C@@H](Nc1cnc(C2CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(2- cyclopropyl-5-((1-(dibenzo [b,d]furan-2-yl)ethyl)amino)- 6-oxo-pyrimidin-1(6H)-yl)- acetamide |
| 343 | 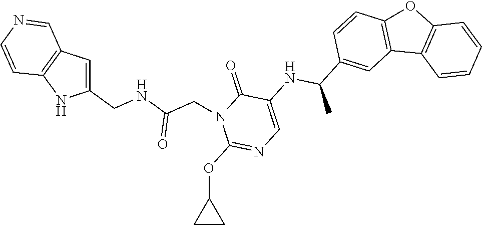 [SMILES: C[C@@H](Nc1cnc(OC2CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(2- cyclopropoxy-5-((1-(dibenzo [b,d]furan-2-yl)ethyl)amino)- 6-oxopyrimidin-1(6H)-yl)acet- amide |
| 344 | 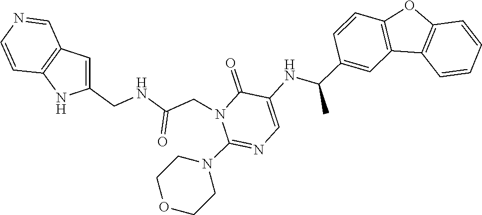 [SMILES: C[C@@H](Nc1cnc(N2CCOCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-morpholino-6-oxo- pyrimidin-1(6H)-yl)acetamide |
| 345 | 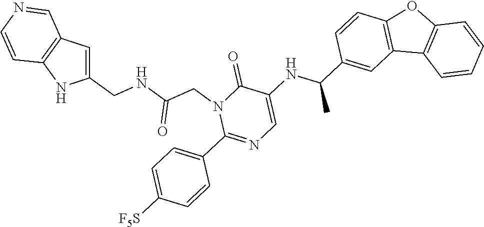 | (R)-N-((1H-pyrrolo[3,2-c] pyridin-2-yl)methyl)-2-(5-((1- (dibenzo[b,d]furan-2-yl)ethyl)- amino)-6-oxo-2-(4-(penta- fluoro-lambda6-sulfaneyl)- phenyl)pyrimidin-1(6H)-yl)- acetamide |
| 346 | 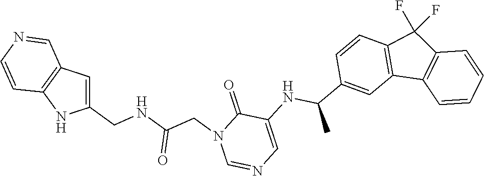 [SMILES: C[C@@H](Nc1cncn(CC(=O)NCc2cc3cnccc3[nH]2)c1=O)c1ccc2c(c1)-c1ccccc1C2(F)F] | (R)-N-((1H-pyrrolo[3,2-c] pyridin-2-yl)methyl)-2-(5-((1- (9,9-difluoro-9H-fluoren-3-yl)- ethyl)amino)-6-oxopyrimidin- 1(6H)-yl)acetamide |
| 347 | 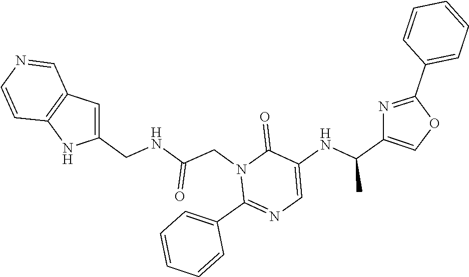 [SMILES: C[C@@H](Nc1cnc(-c2ccccc2)n(CC(=O)NCc2cc3cnccc3[nH]2)c1=O)c1coc(-c2ccccc2)n1] | (R)-N-((1H-pyrrolo[3,2-c] pyridin-2-yl)methyl)-2-(6-oxo- 2-phenyl-5-((1-(2-phenyloxa- zol-4-yl)ethyl)amino)pyrimi- din-1(6H)-yl)acetamide |
| 348 | 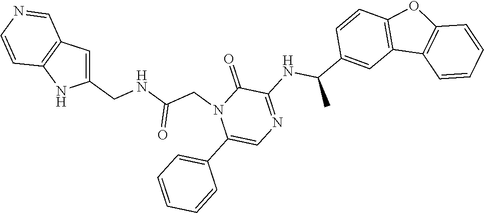 [SMILES: C[C@@H](Nc1ncc(-c2ccccc2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxo-6-phenylpyr- azin-1(2H)-yl)acetamide |
| 349 | 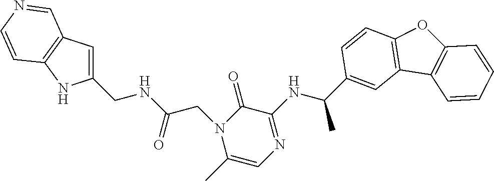 [SMILES: Cc1cnc(N[C@H](C)c2ccc3oc4ccccc4c3c2)c(=O)n1CC(=O)NCc1cc2cnccc2[nH]1] | (R)-N-((1H-pyrrolo[3,2-c] pyridin-2-yl)methyl)-2-(3-((1- (dibenzo[b,d]furan-2-yl)ethyl)- amino)-6-methyl-2-oxopyr- azin-1(2H)-yl)acetamide |
| 350 | 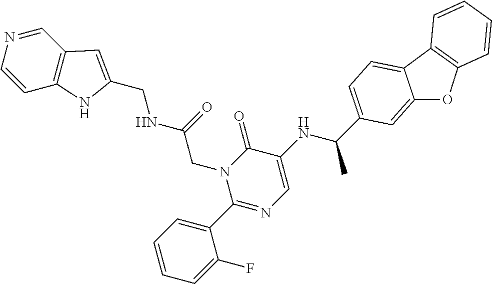 [SMILES: C[C@@H](Nc1cnc(-c2ccccc2F)n(CC(=O)NCc2cc3cnccc3[nH]2)c1=O)c1ccc2c(c1)oc1ccccc12] | (R)-N-((1H-pyrrolo[3,2-c] pyridin-2-yl)methyl)-2-(5-((1- (dibenzo[b,d]furan-3-yl)ethyl)- amino)-2-(2-fluorophenyl)-6- oxopyrimidin-1(6H)-yl)acet- amide |
| 351 | 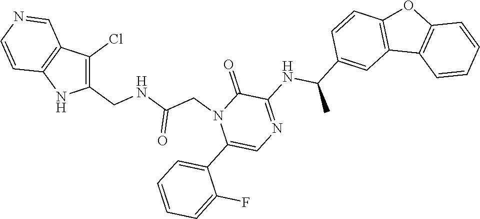 [SMILES: C[C@@H](Nc1ncc(-c2ccccc2F)n(CC(=O)NCc2[nH]c3ccncc3c2Cl)c1=O)c1ccc2oc3ccccc3c2c1] | (R)-N-((3-chloro-1H-pyrrolo [3,2-c]pyridin-2-yl)methyl)-2- (3-((1-(dibenzo[b,d]furan-2- yl)ethyl)amino)-6-(2-fluoro- phenyl)-2-oxopyrazin-1(2H)- yl)acetamide |
| 352 | 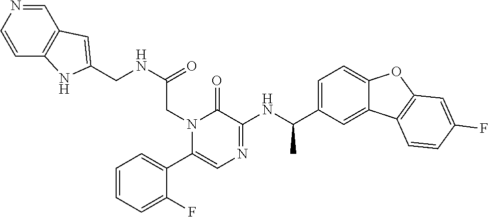 [SMILES: C[C@@H](Nc1ncc(-c2ccccc2F)n(CC(=O)NCc2cc3cnccc3[nH]2)c1=O)c1ccc2oc3cc(F)ccc3c2c1] | (R)-N-((1H-pyrrolo[3,2-c] pyridin-2-yl)methyl)-2-(5-((1- (7-fluorodibenzo[b,d]furan-2- yl)ethyl)amino)-2-(2-fluoro- phenyl)-6-oxopyrimidin-1(6H)- yl)acetamide |
| 353 | 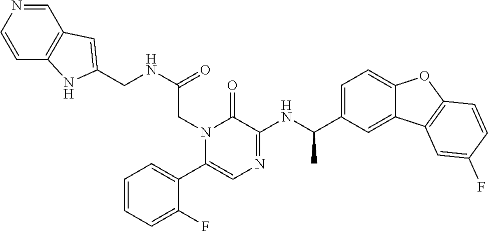 [SMILES: C[C@@H](Nc1ncc(-c2ccccc2F)n(CC(=O)NCc2cc3cnccc3[nH]2)c1=O)c1ccc2oc3ccc(F)cc3c2c1] | (R)-N-((1H-pyrrolo[3,2-c] pyridin-2-yl)methyl)-2-(5-((1- (8-fluorodibenzo[b,d]furan-2- yl)ethyl)amino)-2-(2-fluoro- phenyl)-6-oxopyrimidin-1(6H)- yl)acetamide |
| 354 | 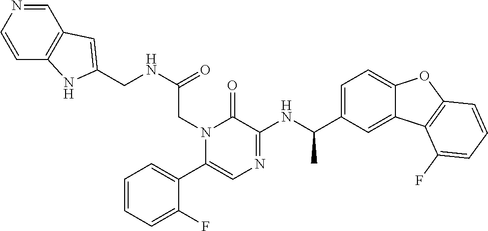 [SMILES: C[C@@H](Nc1ncc(-c2ccccc2F)n(CC(=O)NCc2cc3cnccc3[nH]2)c1=O)c1ccc2oc3cccc(F)c3c2c1] | (R)-N-((1H-pyrrolo[3,2-c] pyridin-2-yl)methyl)-2-(5-((1- (9-fluorodibenzo[b,d]furan-2- yl)ethyl)amino)-2-(2-fluoro- phenyl)-6-oxopyrimidin-1(6H)- yl)acetamide |
| 355 | 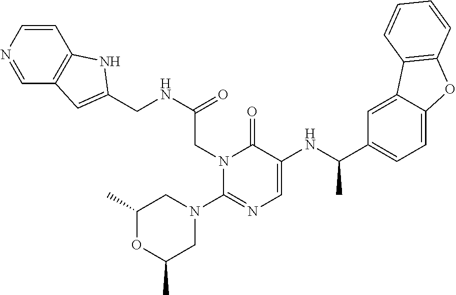 [SMILES: C[C@@H]1CN(c2ncc(N[C@H](C)c3ccc4oc5ccccc5c4c3)c(=O)n2CC(=O)NCc2cc3cnccc3[nH]2)C[C@@H](C)O1] | N-((1H-pyrrolo[3,2-c]pyridin- 2-yl)methyl)-2-(5-(((R)-1- (dibenzo[b,d]furan-2-yl)ethyl)- amino)-2-((2R,6R)-2,6-dimeth- ylmorpholino)-6-oxopyrimidin- 1(6H)-yl)acetamide |
| 356 | 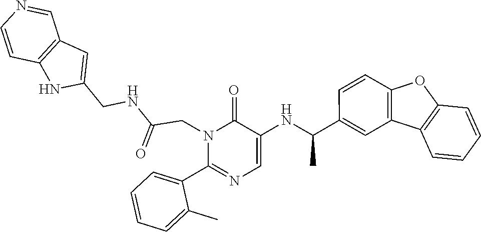 [SMILES: Cc1ccccc1-c1ncc(N[C@H](C)c2ccc3oc4ccccc4c3c2)c(=O)n1CC(=O)NCc1cc2cnccc2[nH]1] | (R)-N-((1H-pyrrolo[3,2-c] pyridin-2-yl)methyl)-2-(5-((1- (dibenzo[b,d]furan-2-yl)ethyl)- amino)-6-oxo-2-(o-tolyl)pyr- imidin-1(6H)-yl)acetamide |
| 357 | 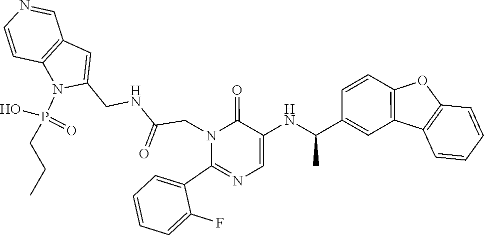 [SMILES: CCCP(=O)(O)n1c(CNC(=O)Cn2c(-c3ccccc3F)ncc(N[C@H](C)c3ccc4oc5ccccc5c4c3)c2=O)cc2cnccc21] | (2-((2-(5-(((R)-1-(dibenzo[b,d] furan-2-yl)ethyl)amino)-2-(2- fluorophenyl)-6-oxopyrimidin- 1(6H)-yl)acetamido)methyl)- 1H-pyrrolo[3,2-c]pyridin-1-yl)- (propyl)phosphinic acid |
| 358 | 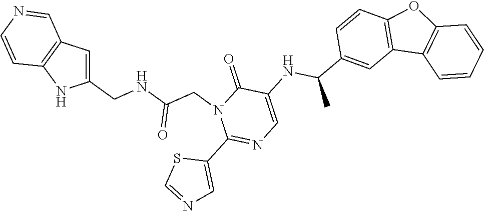 [SMILES: C[C@@H](Nc1cnc(-c2cncs2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(thiazol-5-yl)- pyrimidin-1(6H)-yl)acetamide |
| 359 | 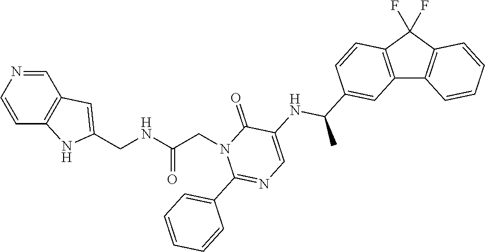 [SMILES: C[C@@H](Nc1cnc(-c2ccccc2)n(CC(=O)NCc2cc3cnccc3[nH]2)c1=O)c1ccc2c(c1)-c1ccccc1C2(F)F] | (R)-N-((1H-pyrrolo[3,2-c] pyridin-2-yl)methyl)-2-(5-((1- (9,9-difluoro-9H-fluoren-3-yl)- ethyl)amino)-6-oxo-2-phenyl- pyrimidin-1(6H)-yl)acetamide |
| 360 | 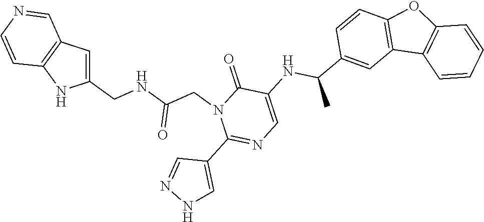 [SMILES: C[C@@H](Nc1cnc(-c2cn[nH]c2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(1H-pyrazol- 4-yl)pyrimidin-1(6H)-yl)acet- amide |
| 361 | 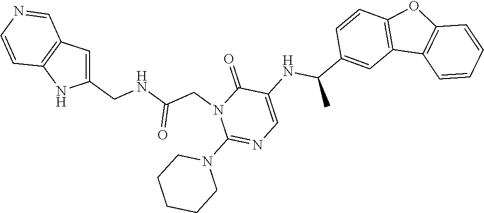 [SMILES: C[C@@H](Nc1cnc(N2CCCCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(piperidin-1- yl)pyrimidin-1(6H)-yl)acet- amide |
| 362 | 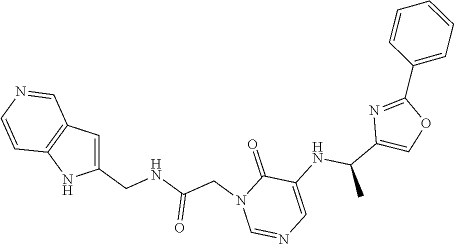 [SMILES: C[C@@H](Nc1cncn(CC(=O)NCc2cc3cnccc3[nH]2)c1=O)c1coc(-c2ccccc2)n1] | (R)-N-((1H-pyrrolo[3,2-c] pyridin-2-yl)methyl)-2-(6-oxo- 5-((1-(2-phenyloxazol-4-yl)- ethyl)amino)pyrimidin-1(6H)- yl)acetamide |
| 363 | 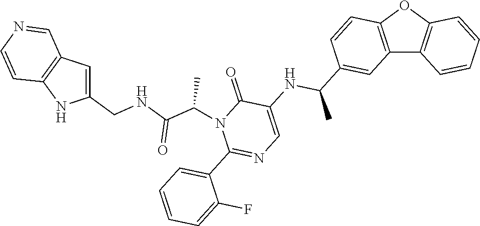 [SMILES: C[C@@H](Nc1cnc(-c2ccccc2F)n([C@@H](C)C(=O)NCc2cc3cnccc3[nH]2)c1=O)c1ccc2oc3ccccc3c2c1] | (S)-N-((1H-pyrrolo[3,2-c] pyridin-2-yl)methyl)-2-(5- (((R)-1-(dibenzo[b,d]furan-2- yl)ethyl)amino)-2-(2-fluoro- phenyl)-6-oxopyrimidin-1(6H)- yl)propanamide |
| 364 | 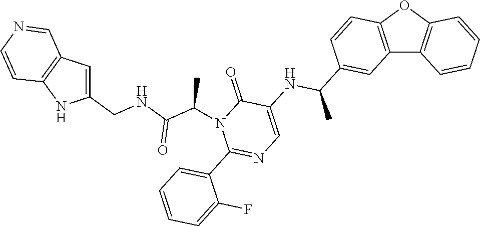 [SMILES: C[C@H](C(=O)NCc1cc2cnccc2[nH]1)n1c(-c2ccccc2F)ncc(N[C@H](C)c2ccc3oc4ccccc4c3c2)c1=O] | (R)-N-((1H-pyrrolo[3,2-c] pyridin-2-yl)methyl)-2-(5- (((R)-1-(dibenzo[b,d]furan-2- yl)ethyl)amino)-2-(2-fluoro- phenyl)-6-oxopyrimidin-1(6H)- yl)propanamide |
| 365 | 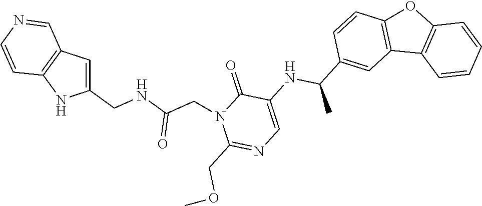 [SMILES: COCc1ncc(N[C@H](C)c2ccc3oc4ccccc4c3c2)c(=O)n1CC(=O)NCc1cc2cnccc2[nH]1] | (R)-N-((1H-pyrrolo[3,2-c] pyridin-2-yl)methyl)-2-(5-((1- (dibenzo[b,d]furan-2-yl)ethyl)- amino)-2-(methoxymethyl)-6- oxopyrimidin-1(6H)-yl)acet- amide |
| 366 | 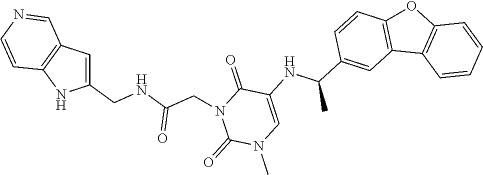 [SMILES: C[C@@H](Nc1cn(C)c(=O)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-3-methyl-2,6-dioxo- 3,6-dihydropyrimidin-1(2H)- yl)acetamide |
| 367 | 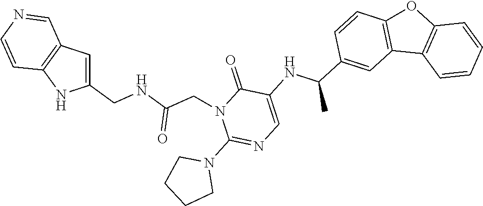 [SMILES: C[C@@H](Nc1cnc(N2CCCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(pyrrolidin-1- yl)pyrimidin-1(6H)-yl)acet- amide |
| 368 | 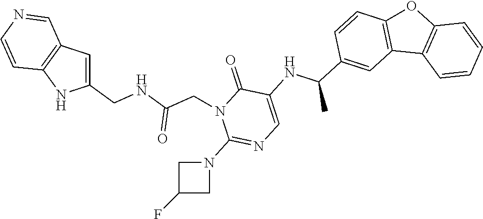 [SMILES: C[C@@H](Nc1cnc(N2CC(F)C2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(3-fluoroazetidin-1- yl)-6-oxopyrimidin-1(6H)-yl)- acetamide |
| 369 | 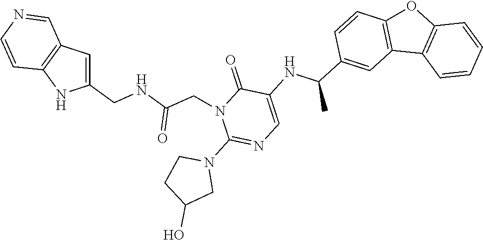 [SMILES: C[C@@H](Nc1cnc(N2CCC(O)C2)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(5-(((R)-1- (dibenzo[b,d]furan-2-yl)ethyl)- amino)-2-(3-hydroxypyrroli- din-1-yl)-6-oxopyrimidin- 1(6H)-yl)acetamide |
| 370 | 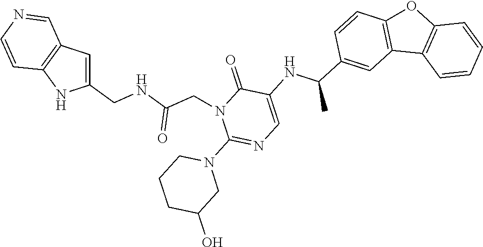 [SMILES: C[C@@H](Nc1cnc(N2CCCC(O)C2)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(5-(((R)-1- (dibenzo[b,d]furan-2-yl)ethyl)- amino)-2-(3-hydroxypiperidin- 1-yl)-6-oxopyrimidin-1(6H)- yl)acetamide |
| 371 | 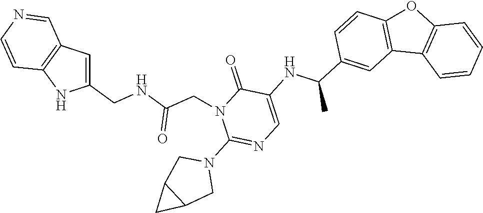 [SMILES: C[C@@H](Nc1cnc(N2CC3CC3C2)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(2-(3-azabicyclo [3.1.0]hexan-3-yl)-5-(((R)-1- (dibenzo[b,d]furan-2-yl)ethyl)- amino)-6-oxopyrimidin-1(6H)- yl)acetamide |
| 372 | 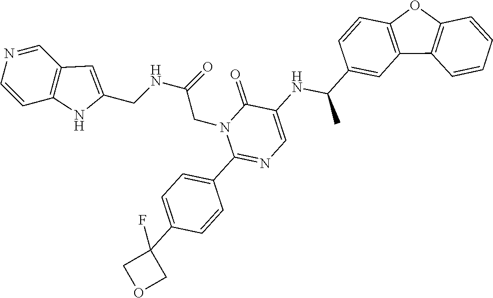 [SMILES: C[C@@H](Nc1cnc(-c2ccc(C3(F)COC3)cc2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(4-(3-fluorooxetan- 3-yl)phenyl)-6-oxopyrimidin- 1(6H)-yl)acetamide |
| 373 | 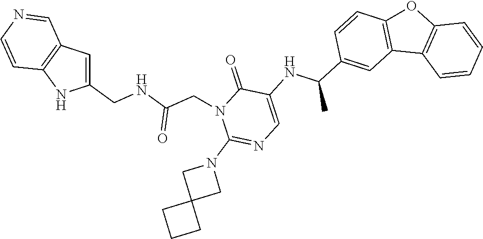 [SMILES: C[C@@H](Nc1cnc(N2CC3(CCC3)C2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(2-azaspiro [3.3]heptan-2-yl)pyrimidin- 1(6H)-yl)acetamide |
| 374 | 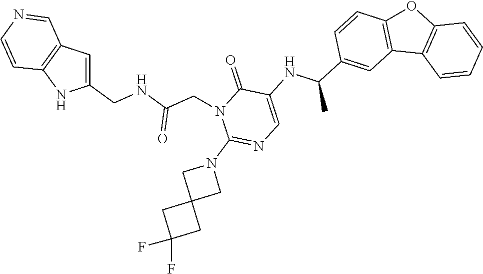 [SMILES: C[C@@H](Nc1cnc(N2CC3(C2)CC(F)(F)C3)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(6,6-difluoro-2-aza- spiro[3.3]heptan-2-yl)-6-oxo- pyrimidin-1(6H)-yl)acetamide |
| 375 | 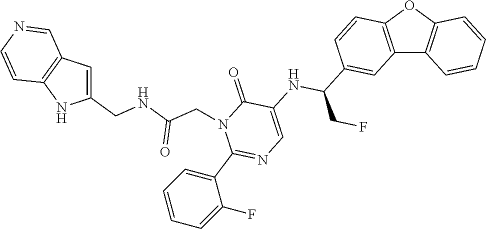 [SMILES: O=C(Cn1c(-c2ccccc2F)ncc(N[C@H](CF)c2ccc3oc4ccccc4c3c2)c1=O)NCc1cc2cnccc2[nH]1] | (S)-N-((1H-pyrrolo[3,2-c] pyridin-2-yl)methyl)-2-(5-((1- (dibenzo[b,d]furan-2-yl)-2- fluoroethyl)amino)-2-(2-fluoro- phenyl)-6-oxopyrimidin-1(6H)- yl)acetamide |
| 376 | 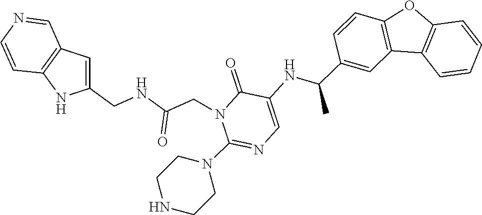 [SMILES: C[C@@H](Nc1cnc(N2CCNCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(piperazin-1- yl)pyrimidin-1(6H)-yl)acet- amide |
| 377 | 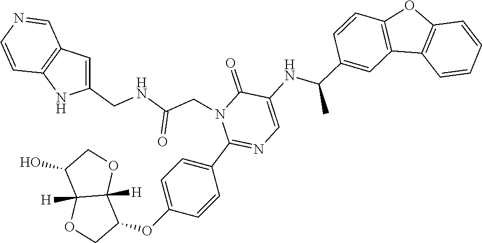 [SMILES: C[C@@H](Nc1cnc(-c2ccc(O[C@@H]3CO[C@H]4[C@@H]3OC[C@H]4O)cc2)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(5-(((R)-1- (dibenzo[b,d]furan-2-yl)ethyl)- amino)-2-(4-(((3R,3aR,6R, 6aR)-6-hydroxyhexahydrofuro [3,2-b]furan-3-yl)oxy)phenyl)- 6-oxopyrimidin-1(6H)-yl)- acetamide |
| 378 | 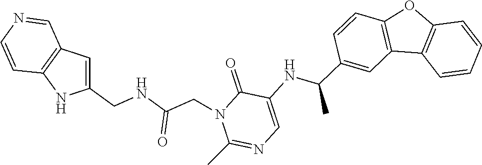 [SMILES: Cc1ncc(N[C@H](C)c2ccc3oc4ccccc4c3c2)c(=O)n1CC(=O)NCc1cc2cnccc2[nH]1] | (R)-N-((1H-pyrrolo[3,2-c] pyridin-2-yl)methyl)-2-(5-((1- (dibenzo[b,d]furan-2-yl)ethyl)- amino)-2-methyl-6-oxopyrimi- din-1(6H)-yl)acetamide |
| 379 | 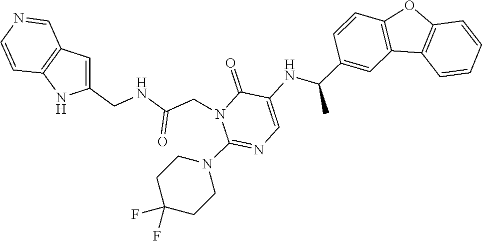 [SMILES: C[C@@H](Nc1cnc(N2CCC(F)(F)CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(4,4-difluoropiperi- din-1-yl)-6-oxopyrimidin- 1(6H)-yl)acetamide |
| 380 | 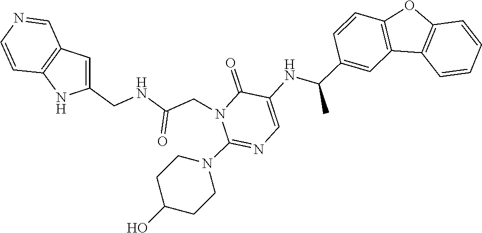 [SMILES: C[C@@H](Nc1cnc(N2CCC(O)CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(4-hydroxypiperidin- 1-yl)-6-oxopyrimidin-1(6H)- yl)acetamide |
| 381 | 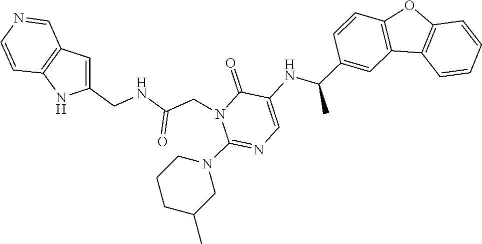 [SMILES: CC1CCCN(c2ncc(N[C@H](C)c3ccc4oc5ccccc5c4c3)c(=O)n2CC(=O)NCc2cc3cnccc3[nH]2)C1] | N-((1H-pyrrolo[3,2-c]pyridin- 2-yl)methyl)-2-(5-(((R)-1- (dibenzo[b,d]furan-2-yl)ethyl)- amino)-2-(3-methylpiperidin- 1-yl)-6-oxopyrimidin-1(6H)- yl)acetamide |
| 382 | 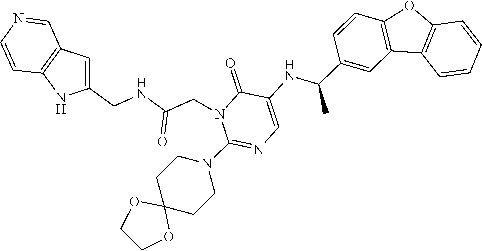 [SMILES: C[C@@H](Nc1cnc(N2CCC3(CC2)OCCO3)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(1,4-dioxa- 8-azaspiro[4.5]decan-8-yl)pyr- imidin-1(6H)-yl)acetamide |
| 383 | 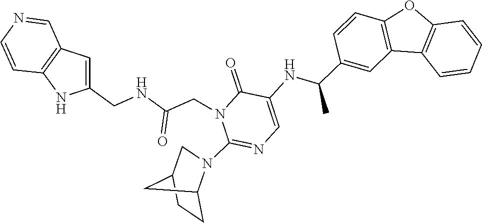 [SMILES: C[C@@H](Nc1cnc(N2CC3CCC2C3)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(2-(2-azabicyclo [2.2.1]heptan-2-yl)-5-(((R)-1- (dibenzo[b,d]furan-2-yl)ethyl)- amino)-6-oxopyrimidin-1(6H)- yl)acetamide |
| 384 | 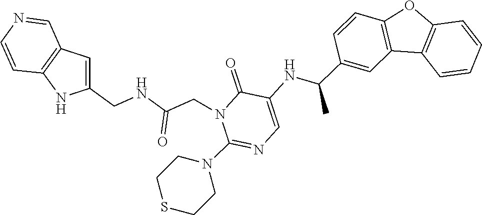 [SMILES: C[C@@H](Nc1cnc(N2CCSCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-thiomorpho- linopyrimidin-1(6H)-yl)acet- amide |
| 385 | 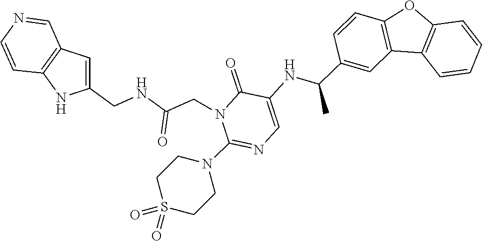 [SMILES: C[C@@H](Nc1cnc(N2CCS(=O)(=O)CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(1,1-dioxidothio- morpholino)-6-oxopyrimidin- 1(6H)-yl)acetamide |
| 386 | 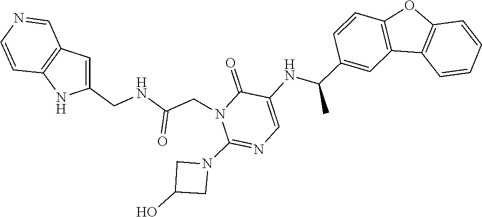 [SMILES: C[C@@H](Nc1cnc(N2CC(O)C2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(3-hydroxyazetidin- 1-yl)-6-oxopyrimidin-1(6H)- yl)acetamide |
| 387 | 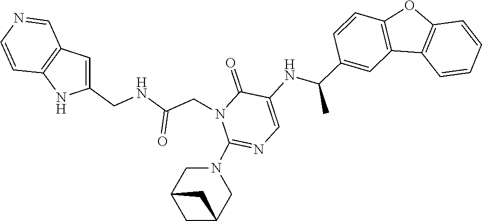 [SMILES: C[C@@H](Nc1cnc(N2C[C@H]3C[C@@H](C2)C3)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(2-((1S,5S)-3- azabicyclo[3.1.1]heptan-3-yl)- 5-(((R)-1-(dibenzo[b,d]furan- 2-yl)ethyl)amino)-6-oxopyr- imidin-1(6H)-yl)acetamide |
| 388 | 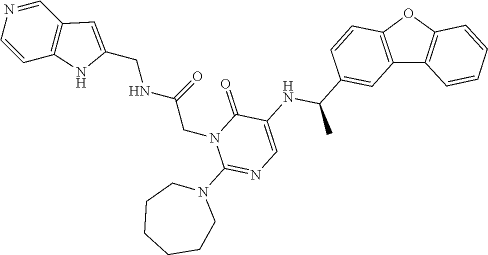 [SMILES: C[C@@H](Nc1cnc(N2CCCCCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(2- (azepan-1-yl)-5-((1-(dibenzo [b,d]furan-2-yl)ethyl)amino)- 6-oxopyrimidin-1(6H)-yl)- acetamide |
| 389 | 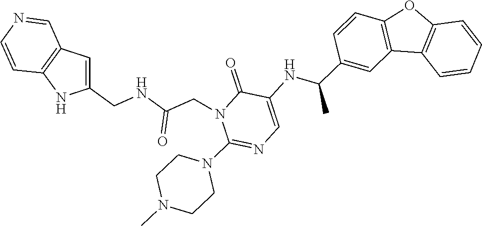 [SMILES: C[C@@H](Nc1cnc(N2CCN(C)CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(4-methylpiperazin- 1-yl)-6-oxopyrimidin-1(6H)- yl)acetamide |
| 390 | 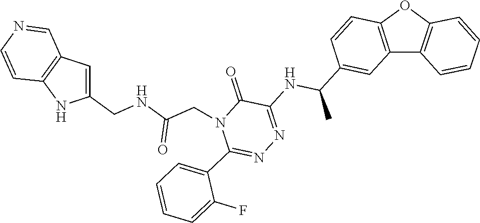 [SMILES: C[C@@H](Nc1nnc(-c2ccccc2F)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(6-((1- (dibenzo[b,d]furan-2-yl)ethyl)- amino)-3-(2-fluorophenyl)-5- oxo-1,2,4-triazin-4(5H)-yl)- acetamide |
| 391 | 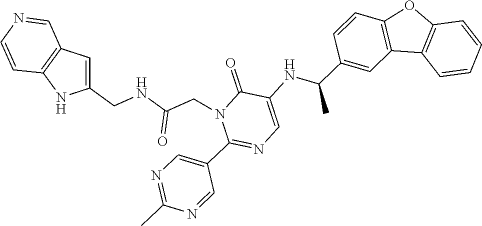 [SMILES: Cc1ncc(-c2ncc(N[C@H](C)c3ccc4oc5ccccc5c4c3)c(=O)n2CC(=O)NCc2cc3cnccc3[nH]2)cn1] | (R)-N-((1H-pyrrolo[3,2-c] pyridin-2-yl)methyl)-2-(5-((1- (dibenzo[b,d]furan-2-yl)ethyl)- amino)-2′-methyl-6-oxo-[2,5′- bipyrimidin]-1(6H)-yl)acet- amide |
| 392 | 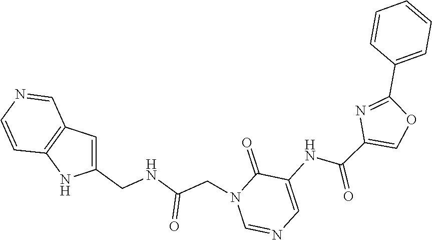 [SMILES: O=C(Cn1cncc(NC(=O)c2coc(-c3ccccc3)n2)c1=O)NCc1cc2cnccc2[nH]1] | N-(1-(2-(((1H-pyrrolo[3,2-c] pyridin-2-yl)methyl)amino)-2- oxoethyl)-6-oxo-1,6-dihydro- pyrimidin-5-yl)-2-phenyl- oxazole-4-carboxamide |
| 393 | 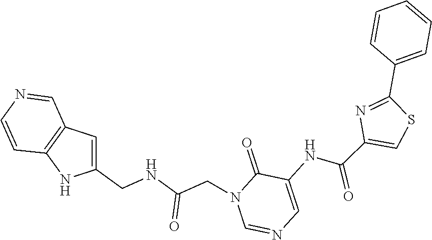 [SMILES: O=C(Cn1cncc(NC(=O)c2csc(-c3ccccc3)n2)c1=O)NCc1cc2cnccc2[nH]1] | N-(1-(2-(((1H-pyrrolo[3,2-c] pyridin-2-yl)methyl)amino)-2- oxoethyl)-6-oxo-1,6-dihydro- pyrimidin-5-yl)-2-phenyl- thiazole-4-carboxamide |
| 394 | 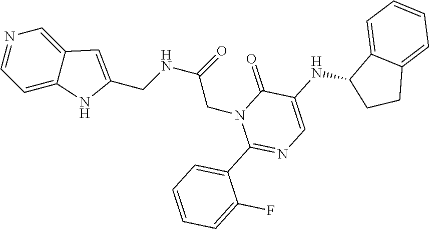 [SMILES: O=C(Cn1c(-c2ccccc2F)ncc(N[C@H]2CCc3ccccc32)c1=O)NCc1cc2cnccc2[nH]1] | (S)-N-((1H-pyrrolo[3,2-c] pyridin-2-yl)methyl)-2-(5- ((2,3-dihydro-1H-inden-1-yl)- amino)-2-(2-fluorophenyl)-6- oxopyrimidin-1(6H)-yl)acet- amide |
| 395 | 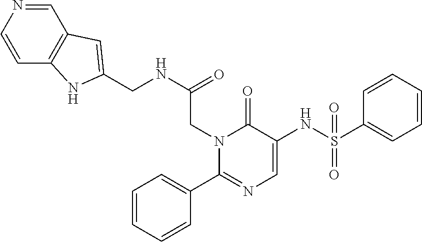 [SMILES: O=C(Cn1c(-c2ccccc2)ncc(NS(=O)(=O)c2ccccc2)c1=O)NCc1cc2cnccc2[nH]1] | N-((1H-pyrrolo[3,2-c]pyridin- 2-yl)methyl)-2-(6-oxo-2-phen- yl-5-(phenylsulfonamido)- pyrimidin-1(6H)-yl)acetamide |
| 396 | 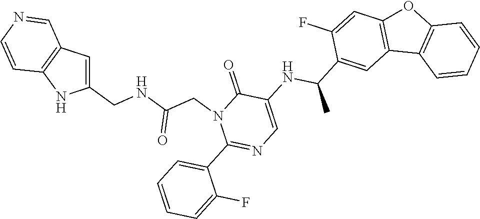 [SMILES: C[C@@H](Nc1cnc(-c2ccccc2F)n(CC(=O)NCc2cc3cnccc3[nH]2)c1=O)c1cc2c(cc1F)oc1ccccc12] | (R)-N-((1H-pyrrolo[3,2-c] pyridin-2-yl)methyl)-2-(5-((1- (3-fluorodibenzo[b,d]furan-2- yl)ethyl)amino)-2-(2-fluoro- phenyl)-6-oxopyrimidin-1(6H)- yl)acetamide |
| 397 | 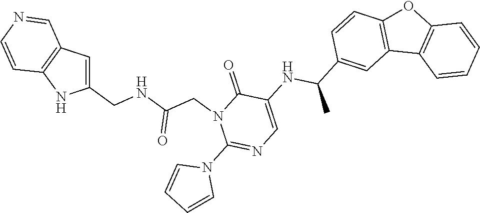 [SMILES: C[C@@H](Nc1cnc(-n2cccc2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(1H-pyrrol-1- yl)pyrimidin-1(6H)-yl)acet- amide |
| 398 | 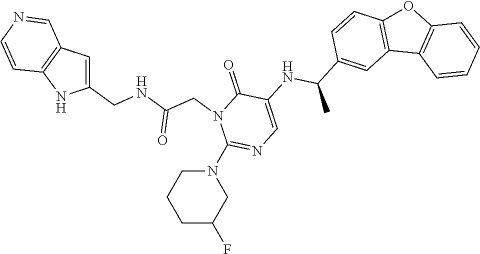 [SMILES: C[C@@H](Nc1cnc(N2CCCC(F)C2)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(5-(((R)-1- (dibenzo[b,d]furan-2-yl)ethyl)- amino)-2-(3-fluoropiperidin-1- yl)-6-oxopyrimidin-1(6H)-yl)- acetamide |
| 399 | 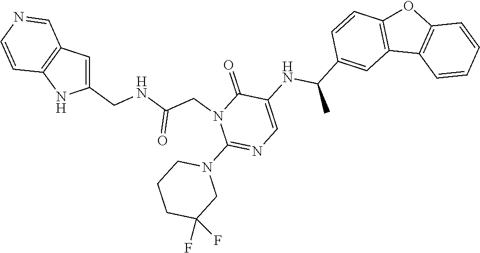 [SMILES: C[C@@H](Nc1cnc(N2CCCC(F)(F)C2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(3,3-difluoropiperi- din-1-yl)-6-oxopyrimidin- 1(6H)-yl)acetamide |
| 400 | 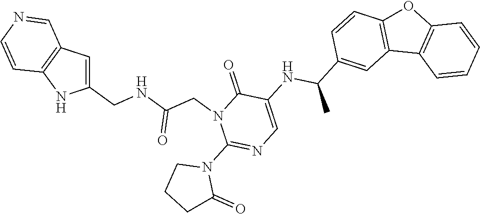 [SMILES: C[C@@H](Nc1cnc(N2CCCC2=O)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-6-oxo-2-(2-oxopyr- rolidin-1-yl)pyrimidin-1(6H)- yl)acetamide |
| 401 | 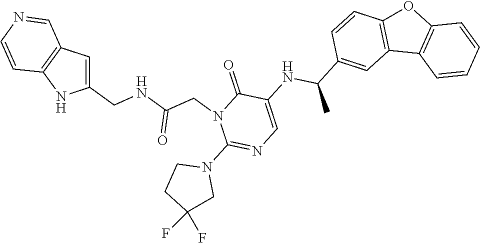 [SMILES: C[C@@H](Nc1cnc(N2CCC(F)(F)C2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(5-((1- (dibenzo[b,d]furan-2-yl)ethyl)- amino)-2-(3,3-difluoropyrroli- din-1-yl)-6-oxopyrimidin- 1(6H)-yl)acetamide |
| 402 | 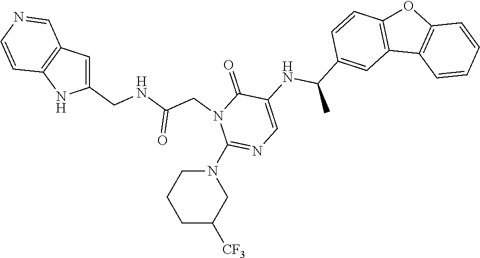 [SMILES: C[C@@H](Nc1cnc(N2CCCC(C(F)(F)F)C2)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(5-(((R)-1- (dibenzo[b,d]furan-2-yl)ethyl)- amino)-6-oxo-2-(3-(trifluoro- methyl)piperidin-1-yl)pyrimi- din-1(6H)-yl)acetamide |
| 403 | 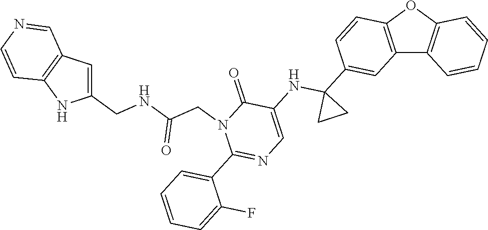 [SMILES: O=C(Cn1c(-c2ccccc2F)ncc(NC2(c3ccc4oc5ccccc5c4c3)CC2)c1=O)NCc1cc2cnccc2[nH]1] | N-((1H-pyrrolo[3,2-c]pyridin- 2-yl)methyl)-2-(5-((1-(dibenzo [b,d]furan-2-yl)cyclopropyl)- amino)-2-(2-fluorophenyl)-6- oxopyrimidin-1(6H)-yl)acet- amide |
| 404 | 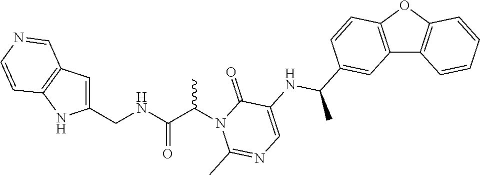 [SMILES: Cc1ncc(N[C@H](C)c2ccc3oc4ccccc4c3c2)c(=O)n1C(C)C(=O)NCc1cc2cnccc2[nH]1] | N-((1H-pyrrolo[3,2-c]pyridin- 2-yl)methyl)-2-(5-(((R)-1- (dibenzo[b,d]furan-2-yl)ethyl)- amino)-2-methyl-6-oxopyrimi- din-1(6H)-yl)propanamide |
| 405 | 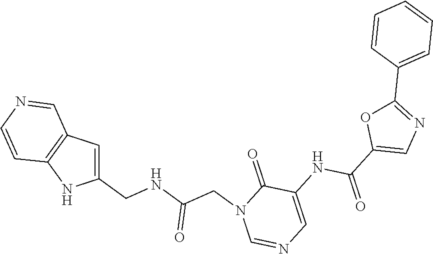 [SMILES: O=C(Cn1cncc(NC(=O)c2cnc(-c3ccccc3)o2)c1=O)NCc1cc2cnccc2[nH]1] | N-(1-(2-(((1H-pyrrolo[3,2-c] pyridin-2-yl)methyl)amino)-2- oxoethyl)-6-oxo-1,6-dihydro- pyrimidin-5-yl)-2-phenyl- oxazole-5-carboxamide |
| 406 | 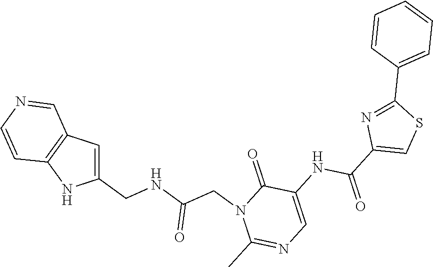 [SMILES: Cc1ncc(NC(=O)c2csc(-c3ccccc3)n2)c(=O)n1CC(=O)NCc1cc2cnccc2[nH]1] | N-(1-(2-(((1H-pyrrolo[3,2-c] pyridin-2-yl)methyl)amino)-2- oxoethyl)-2-methyl-6-oxo-1,6- dihydropyrimidin-5-yl)-2- phenylthiazole-4-carboxamide |
| 407 | 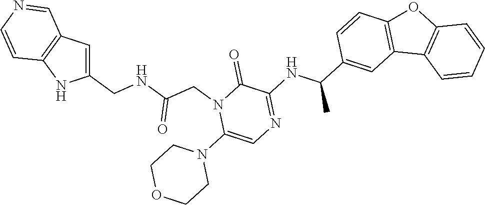 [SMILES: C[C@@H](Nc1ncc(N2CCOCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-6-morpholino-2-oxo- pyrazin-1(2H)-yl)acetamide |
| 408 | 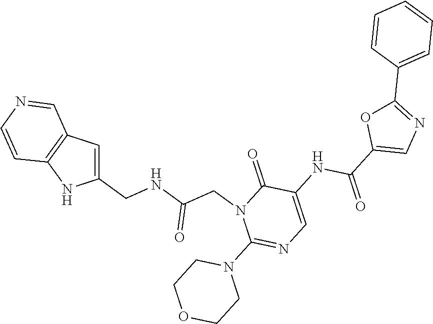 [SMILES: O=C(Cn1c(N2CCOCC2)ncc(NC(=O)c2cnc(-c3ccccc3)o2)c1=O)NCc1cc2cnccc2[nH]1] | N-(1-(2-(((1H-pyrrolo[3,2-c] pyridin-2-yl)methyl)amino)-2- oxoethyl)-2-morpholino-6- oxo-1,6-dihydropyrimidin-5- yl)-2-phenyloxazole-5-carbox- amide |
| 409 | 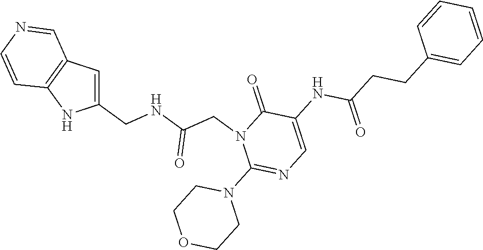 [SMILES: O=C(Cn1c(N2CCOCC2)ncc(NC(=O)CCc2ccccc2)c1=O)NCc1cc2cnccc2[nH]1] | N-(1-(2-(((1H-pyrrolo[3,2-c] pyridin-2-yl)methyl)amino)- 2-oxoethyl)-2-morpholino-6- oxo-1,6-dihydropyrimidin-5- yl)-3-phenylpropanamide |
| 410 | 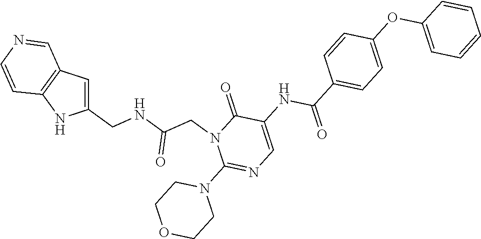 [SMILES: O=C(Cn1c(N2CCOCC2)ncc(NC(=O)c2ccc(Oc3ccccc3)cc2)c1=O)NCc1cc2cnccc2[nH]1] | N-(1-(2-(((1H-pyrrolo[3,2-c] pyridin-2-yl)methyl)amino)-2- oxoethyl)-2-morpholino-6- oxo-1,6-dihydropyrimidin-5- yl)-4-phenoxybenzamide |
| 411 | 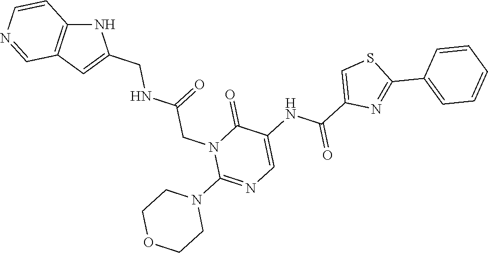 [SMILES: O=C(Cn1c(N2CCOCC2)ncc(NC(=O)c2csc(-c3ccccc3)n2)c1=O)NCc1cc2cnccc2[nH]1] | N-(1-(2-(((1H-pyrrolo[3,2-c] pyridin-2-yl)methyl)amino)- 2-oxoethyl)-2-morpholino-6- oxo-1,6-dihydropyrimidin-5- yl)-2-phenylthiazole-4-carbox- amide |
| 412 | 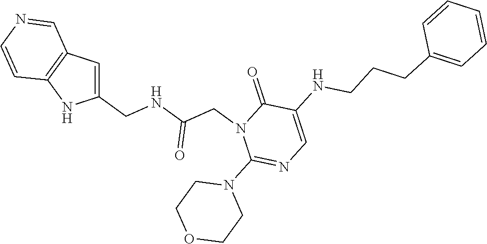 [SMILES: O=C(Cn1c(N2CCOCC2)ncc(NCCCc2ccccc2)c1=O)NCc1cc2cnccc2[nH]1] | N-((1H-pyrrolo[3,2-c]pyridin- 2-yl)methyl)-2-(2-morpholino- 6-oxo-5-((3-phenylpropyl)- amino)pyrimidin-1(6H)-yl)- acetamide |
| 413 | 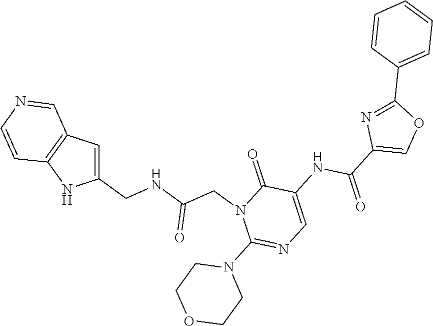 [SMILES: O=C(Cn1c(N2CCOCC2)ncc(NC(=O)c2coc(-c3ccccc3)n2)c1=O)NCc1cc2cnccc2[nH]1] | N-(1-(2-(((1H-pyrrolo[3,2-c] pyridin-2-yl)methyl)amino)- 2-oxoethyl)-2-morpholino-6- oxo-1,6-dihydropyrimidin-5- yl)-2-phenyloxazole-4-carbox- amide |
| 414 | 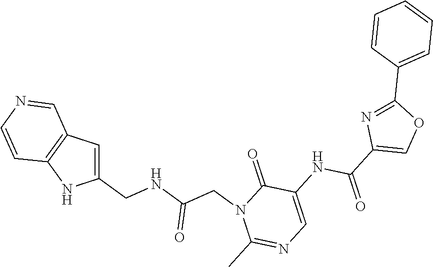 [SMILES: Cc1ncc(NC(=O)c2coc(-c3ccccc3)n2)c(=O)n1CC(=O)NCc1cc2cnccc2[nH]1] | N-(1-(2-(((1H-pyrrolo[3,2-c] pyridin-2-yl)methyl)amino)-2- oxoethyl)-2-methyl-6-oxo-1,6- dihydropyrimidin-5-yl)-2- phenyloxazole-4-carboxamide |
| 415 | 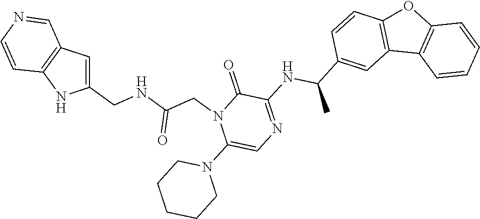 [SMILES: C[C@@H](Nc1ncc(N2CCCCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxo-6-(piperidin-1- yl)pyrazin-1(2H)-yl)acetamide |
| 416 | 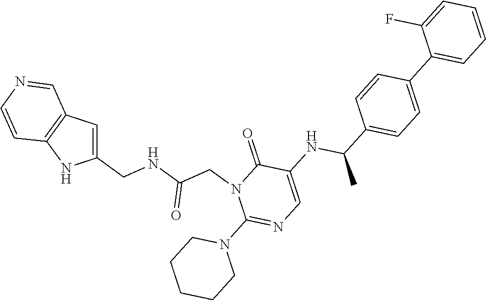 [SMILES: C[C@@H](Nc1cnc(N2CCCCC2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(5-((1- (2′-fluoro-[1,1′-biphenyl]-4- yl)ethyl)amino)-6-oxo-2- (piperidin-1-yl)pyrimidin- 1(6H)-yl)acetamide |
| 417 | 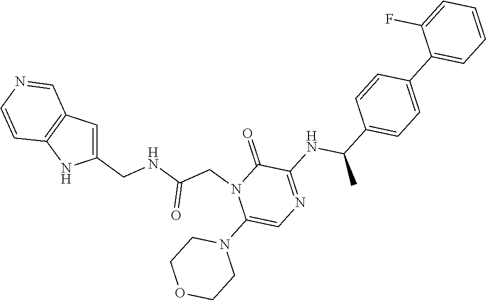 [SMILES: C[C@@H](Nc1ncc(N2CCOCC2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(3-((1- (2′-fluoro-[1,1′-biphenyl]-4- yl)ethyl)amino)-6-morpholino- 2-oxopyrazin-1(2H)-yl)acet- amide |
| 418 | 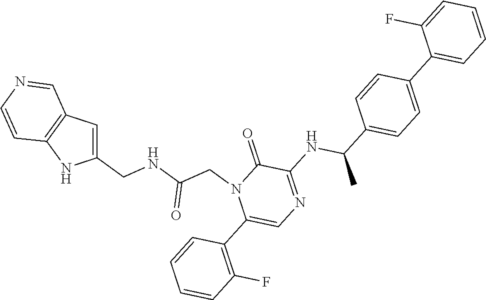 [SMILES: C[C@@H](Nc1ncc(-c2ccccc2F)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(3-((1- (2′-fluoro-[1,1′-biphenyl]-4- yl)ethyl)amino)-6-(2-fluoro- phenyl)-2-oxopyrazin-1(2H)- yl)acetamide |
| 419 | 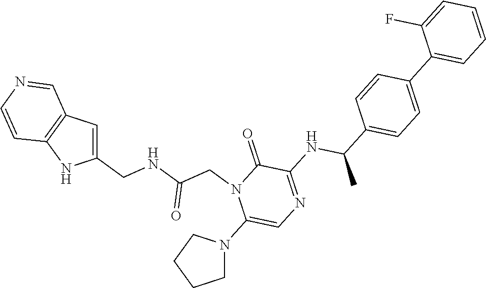 [SMILES: C[C@@H](Nc1ncc(N2CCCC2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(3-((1- (2′-fluoro-[1,1′-biphenyl]-4- yl)ethyl)amino)-2-oxo-6-(pyr- rolidin-1-yl)pyrazin-1(2H)- yl)acetamide |
| 420 | 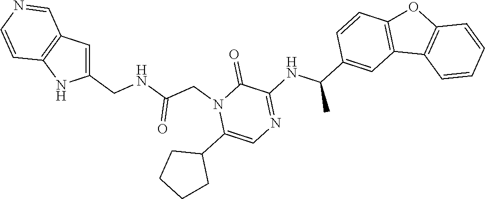 [SMILES: C[C@@H](Nc1ncc(C2CCCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(6- cyclopentyl-3-((1-(dibenzo [b,d]furan-2-yl)ethyl)amino)- 2-oxopyrazin-1(2H)-yl)acet- amide |
| 421 | 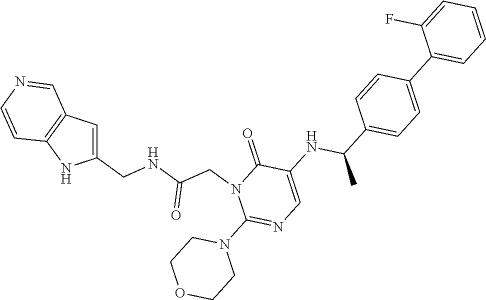 [SMILES: C[C@@H](Nc1cnc(N2CCOCC2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(5-((1- (2′-fluoro-[1,1′-biphenyl]-4-yl)- ethyl)amino)-2-morpholino-6- oxopyrimidin-1(6H)-yl)acet- amide |
| 422 | 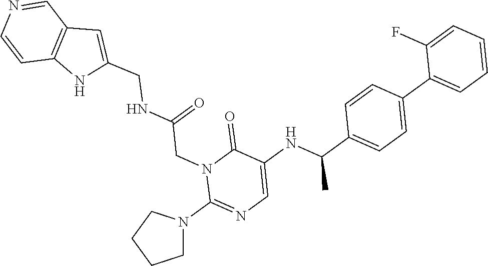 [SMILES: C[C@@H](Nc1cnc(N2CCCC2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(5-((1- (2′-fluoro-[1,1′-biphenyl]-4- yl)ethyl)amino)-6-oxo-2-(pyr- rolidin-1-yl)pyrimidin-1(6H)- yl)acetamide |
| 423 | 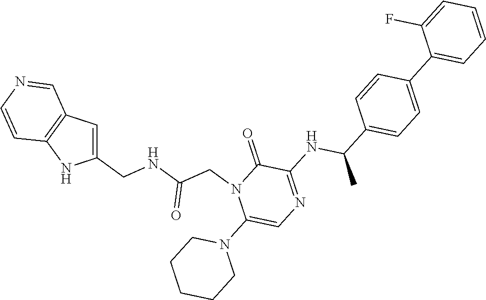 [SMILES: C[C@@H](Nc1ncc(N2CCCCC2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(3- ((1-(2′-fluoro-[1,1′-biphenyl]- 4-yl)ethyl)amino)-2-oxo-6- (piperidin-1-yl)pyrazin-1(2H)- yl)acetamide |
| 424 | 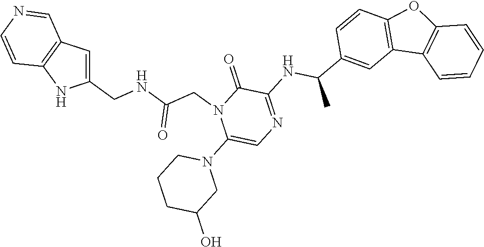 [SMILES: C[C@@H](Nc1ncc(N2CCCC(O)C2)n(CC(=O)NCc2cc3cnccc3[nH]2)c1=O)c1ccc2oc3ccccc3c2c1] | N-((1H-pyrrolo[3,2-c]pyridin- 2-yl)methyl)-2-(3-(((R)-1- (dibenzo[b,d]furan-2-yl)ethyl)- amino)-6-(3-hydroxypiperidin- 1-yl)-2-oxopyrazin-1(2H)-yl)- acetamide |
| 425 | 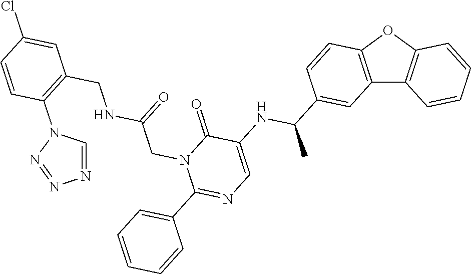 [SMILES: C[C@@H](Nc1cnc(-c2ccccc2)n(CC(=O)NCc2cc(Cl)ccc2-n2cnnn2)c1=O)c1ccc2oc3ccccc3c2c1] | (R)-N-(5-chloro-2-(1H-tetra- zol-1-yl)benzyl)-2-(5-((1- (dibenzo[b,d]furan-2-yl)ethyl)- amino)-6-oxo-2-phenylpyrimi- din-1(6H)-yl)acetamide |
| 426 | 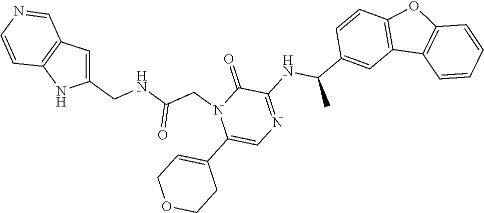 [SMILES: C[C@@H](Nc1ncc(C2=CCOCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-6-(3,6-dihydro-2H- pyran-4-yl)-2-oxopyrazin- 1(2H)-yl)acetamide |
| 427 | 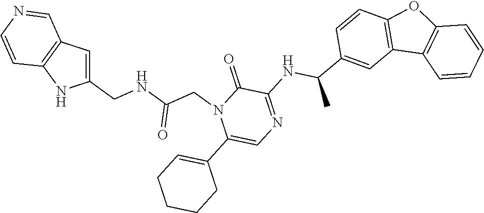 [SMILES: C[C@@H](Nc1ncc(C2=CCCCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(6- (cyclohex-1-en-1-yl)-3-((1- (dibenzo[b,d]furan-2-yl)ethyl)- amino)-2-oxopyrazin-1(2H)- yl)acetamide |
| 428 | 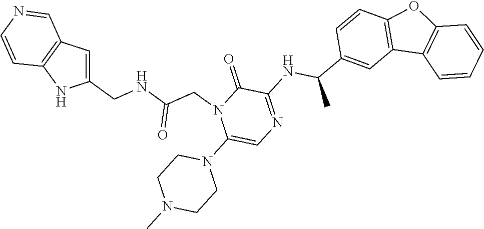 [SMILES: C[C@@H](Nc1ncc(N2CCN(C)CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-6-(4-methylpiperazin- 1-yl)-2-oxopyrazin-1(2H)-yl)- acetamide |
| 429 | 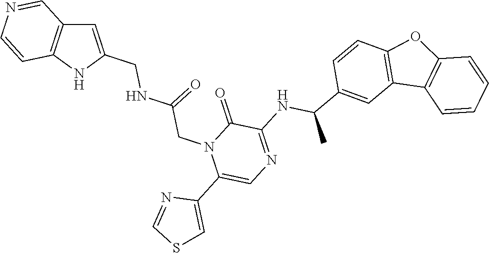 [SMILES: C[C@@H](Nc1ncc(-c2cscn2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxo-6-(thiazol-4-yl)- pyrazin-1(2H)-yl)acetamide |
| 430 | 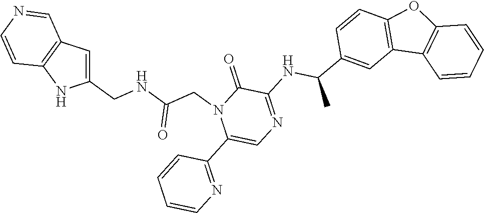 [SMILES: C[C@@H](Nc1ncc(-c2ccccn2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxo-6-(pyridin-2-yl)- pyrazin-1(2H)-yl)acetamide |
| 431 | 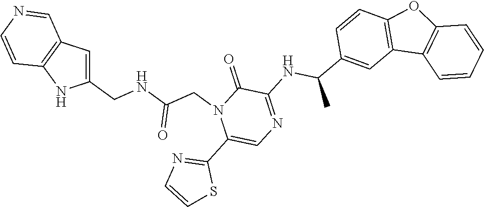 [SMILES: C[C@@H](Nc1ncc(-c2nccs2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxo-6-(thiazol-2-yl)- pyrazin-1(2H)-yl)acetamide |
| 432 | 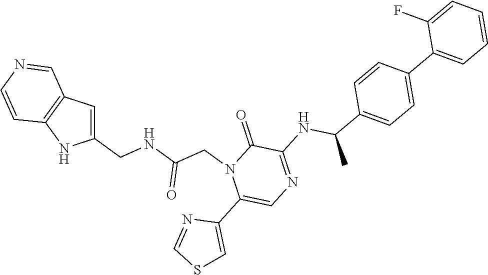 [SMILES: C[C@@H](Nc1ncc(-c2cscn2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(3-((1- (2′-fluoro-[1,1′-biphenyl]-4- yl)ethyl)amino)-2-oxo-6- (thiazol-4-yl)pyrazin-1(2H)- yl)acetamide |
| 433 | 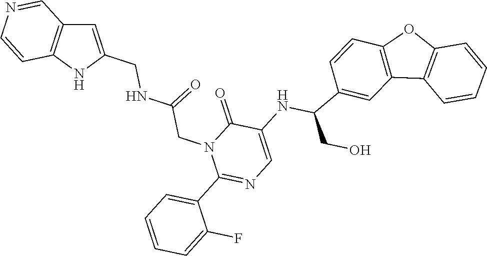 [SMILES: O=C(Cn1c(-c2ccccc2F)ncc(N[C@H](CO)c2ccc3oc4ccccc4c3c2)c1=O)NCc1cc2cnccc2[nH]1] | (S)-N-((1H-pyrrolo[3,2-c] pyridin-2-yl)methyl)-2-(5- ((1-(dibenzo[b,d]furan-2-yl)- 2-hydroxyethyl)amino)-2-(2- fluorophenyl)-6-oxopyrimi- din-1(6H)-yl)acetamide |
| 434 | 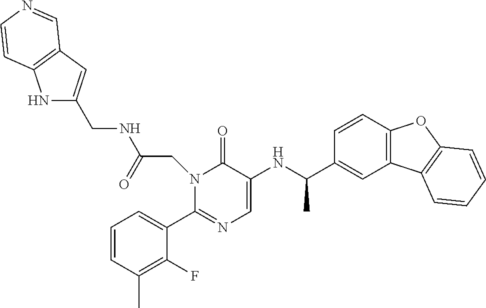 [SMILES: Cc1cccc(-c2ncc(N[C@H](C)c3ccc4oc5ccccc5c4c3)c(=O)n2CC(=O)NCc2cc3cnccc3[nH]2)c1F] | (R)-N-((1H-pyrrolo[3,2-c] pyridin-2-yl)methyl)-2-(5- ((1-(dibenzo[b,d]furan-2- yl)ethyl)amino)-2-(2-fluoro- 3-methylphenyl)-6-oxopyr- imidin-1(6H)-yl)acetamide |
| 435 | 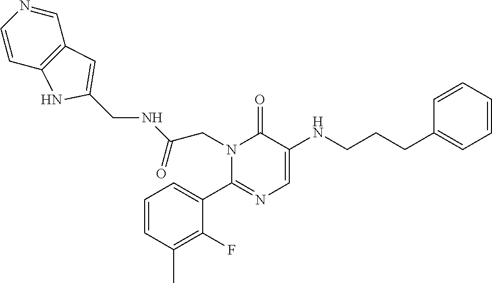 [SMILES: Cc1cccc(-c2ncc(NCCCc3ccccc3)c(=O)n2CC(=O)NCc2cc3cnccc3[nH]2)c1F] | N-((1H-pyrrolo[3,2-c]pyridin- 2-yl)methyl)-2-(2-(2-fluoro-3- methylphenyl)-6-oxo-5-((3- phenylpropyl)amino)pyrimi- din-1(6H)-yl)acetamide |
| 436 | 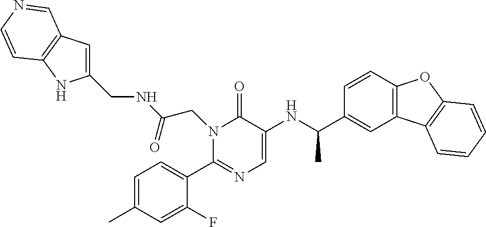 [SMILES: Cc1ccc(-c2ncc(N[C@H](C)c3ccc4oc5ccccc5c4c3)c(=O)n2CC(=O)NCc2cc3cnccc3[nH]2)c(F)c1] | (R)-N-((1H-pyrrolo[3,2-c] pyridin-2-yl)methyl)-2-(5-((1- (dibenzo[b,d]furan-2-yl)ethyl)- amino)-2-(2-fluoro-4-methyl- phenyl)-6-oxopyrimidin-1(6H)- yl)acetamide |
| 437 | 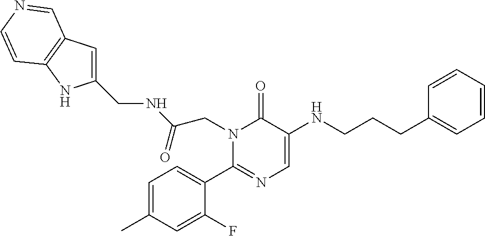 [SMILES: Cc1ccc(-c2ncc(NCCCc3ccccc3)c(=O)n2CC(=O)NCc2cc3cnccc3[nH]2)c(F)c1] | N-((1H-pyrrolo[3,2-c]pyridin- 2-yl)methyl)-2-(2-(2-fluoro-4- methylphenyl)-6-oxo-5-((3- phenylpropyl)amino)pyrimi- din-1(6H)-yl)acetamide |
| 438 | 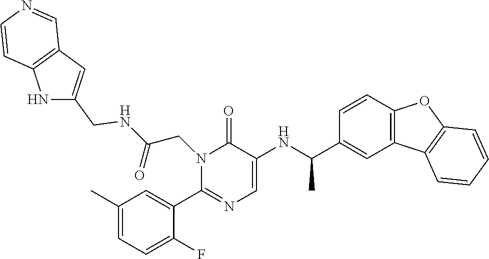 [SMILES: Cc1ccc(F)c(-c2ncc(N[C@H](C)c3ccc4oc5ccccc5c4c3)c(=O)n2CC(=O)NCc2cc3cnccc3[nH]2)c1] | (R)-N-((1H-pyrrolo[3,2-c] pyridin-2-yl)methyl)-2-(5-((1- (dibenzo[b,d]furan-2-yl)ethyl)- amino)-2-(2-fluoro-5-methyl- phenyl)-6-oxopyrimidin-1(6H)- yl)acetamide |
| 439 | 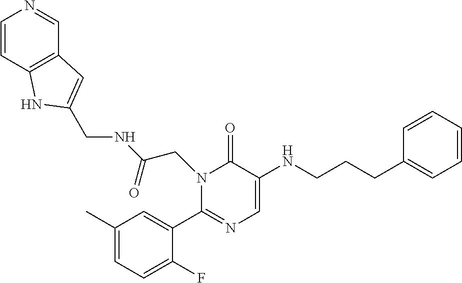 [SMILES: Cc1ccc(F)c(-c2ncc(NCCCc3ccccc3)c(=O)n2CC(=O)NCc2cc3cnccc3[nH]2)c1] | N-((1H-pyrrolo[3,2-c]pyridin- 2-yl)methyl)-2-(2-(2-fluoro-5- methylphenyl)-6-oxo-5-((3- phenylpropyl)amino)pyrimi- din-1(6H)-yl)acetamide |
| 440 | 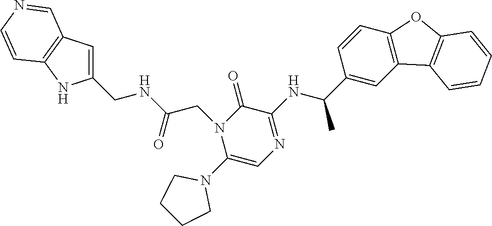 [SMILES: C[C@@H](Nc1ncc(N2CCCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxo-6-(pyrrolidin-1- yl)pyrazin-1(2H)-yl)acetamide |
| 441 | 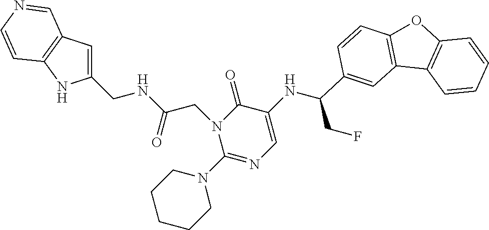 [SMILES: O=C(Cn1c(N2CCCCC2)ncc(N[C@H](CF)c2ccc3oc4ccccc4c3c2)c1=O)NCc1cc2cnccc2[nH]1] | rel-(R)-N-((1H-pyrrolo[3,2-c] pyridin-2-yl)methyl)-2-(5-((1- (dibenzo[b,d]furan-2-yl)-2- fluoroethyl)amino)-6-oxo-2- (piperidin-1-yl)pyrimidin- 1(6H)-yl)acetamide |
| 442 | 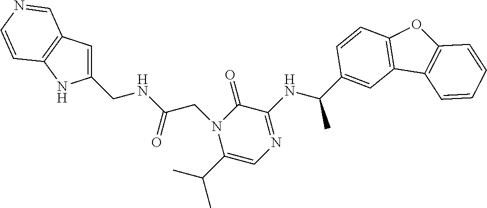 [SMILES: CC(C)c1cnc(N[C@H](C)c2ccc3oc4ccccc4c3c2)c(=O)n1CC(=O)NCc1cc2cnccc2[nH]1] | (R)-N-((1H-pyrrolo[3,2-c] pyridin-2-yl)methyl)-2-(3-((1- (dibenzo[b,d]furan-2-yl)ethyl)- amino)-6-isopropyl-2-oxopyr- azin-1(2H)-yl)acetamide |
| 443 | 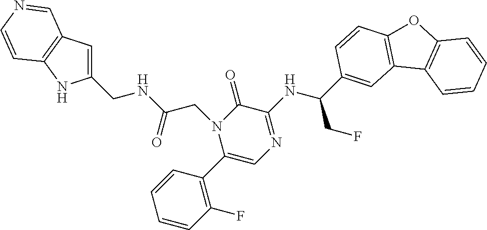 [SMILES: O=C(Cn1c(-c2ccccc2F)cnc(N[C@H](CF)c2ccc3oc4ccccc4c3c2)c1=O)NCc1cc2cnccc2[nH]1] | (S)-N-((1H-pyrrolo[3,2-c] pyridin-2-yl)methyl)-2-(3-((1- (dibenzo[b,d]furan-2-yl)-2- fluoroethyl)amino)-6-(2- fluorophenyl)-2-oxopyrazin- 1(2H)-yl)acetamide |
| 444 | 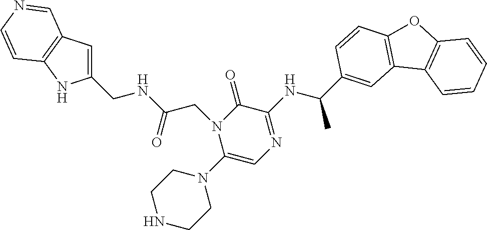 [SMILES: C[C@@H](Nc1ncc(N2CCNCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxo-6-(piperazin-1- yl)pyrazin-1(2H)-yl)acetamide |
| 445 | 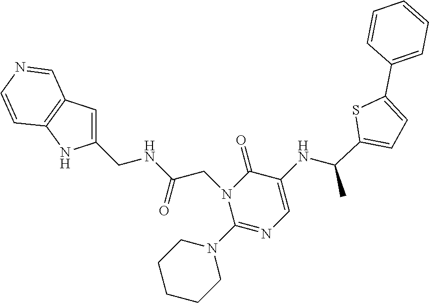 [SMILES: C[C@@H](Nc1cnc(N2CCCCC2)n(CC(=O)NCc2cc3cnccc3[nH]2)c1=O)c1ccc(-c2ccccc2)s1] | (R)-N-((1H-pyrrolo[3,2-c] pyridin-2-yl)methyl)-2-(6- ox0-5-((1-(5-phenylthiophen- 2-yl)ethyl)amino)-2-(piperi- din-1-yl)pyrimidin-1(6H)-yl)- acetamide |
| 446 | 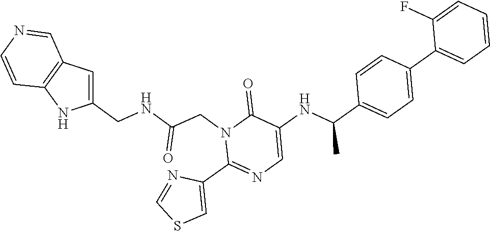 [SMILES: C[C@@H](Nc1cnc(-c2cscn2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(5- ((1-(2′-fluoro-[1,1′-biphenyl]- 4-yl)ethyl)amino)-6-oxo-2- (thiazol-4-yl)pyrimidin-1(6H)- yl)acetamide |
| 447 | 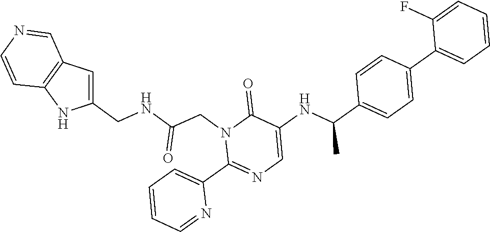 [SMILES: C[C@@H](Nc1cnc(-c2ccccn2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(5- ((1-(2′-fluoro-[1,1′-biphenyl]- 4-yl)ethyl)amino)-6-oxo-2- (pyridin-2-yl)pyrimidin-1(6H)- yl)acetamide |
| 448 | 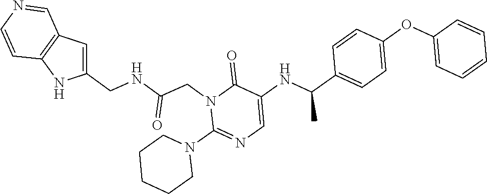 [SMILES: C[C@@H](Nc1cnc(N2CCCCC2)n(CC(=O)NCc2cc3cnccc3[nH]2)c1=O)c1ccc(Oc2ccccc2)cc1] | (R)-N-((1H-pyrrolo[3,2-c] pyridin-2-yl)methyl)-2-(6- oxo-5-((1-(4-phenoxyphenyl)- ethyl)amino)-2-(piperidin-1- yl)pyrimidin-1(6H)-yl)acet- amide |
| 449 | 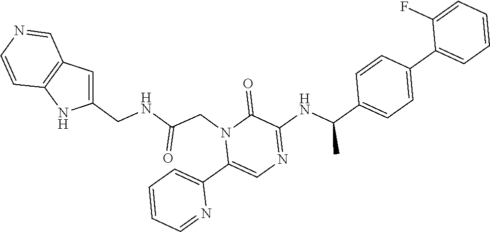 [SMILES: C[C@@H](Nc1ncc(-c2ccccn2)n(CC(=O)NCc2cc3cnccc3[nH]2)c1=O)c1ccc(-c2ccccc2F)cc1] | (R)-N-((1H-pyrrolo[3,2-c] pyridin-2-yl)methyl)-2-(3- ((1-(2′-fluoro-[1,1′-biphenyl]- 4-yl)ethyl)amino)-2-oxo-6- (pyridin-2-yl)pyrazin-1(2H)- yl)acetamide |
| 450 | 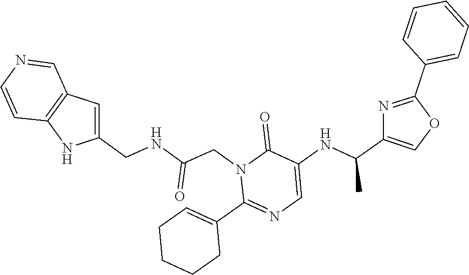 [SMILES: C[C@@H](Nc1cnc(C2=CCCCC2)n(CC(=O)NCc2cc3cnccc3[nH]2)c1=O)c1coc(-c2ccccc2)n1] | (R)-N-((1H-pyrrolo[3,2-c] pyridin-2-yl)methyl)-2-(2- (cyclohex-1-en-1-yl)-6-oxo- 5-((1-(2-phenyloxazol-4-yl)- ethyl)amino)pyrimidin-1(6H)- yl)acetamide |
| 451 | 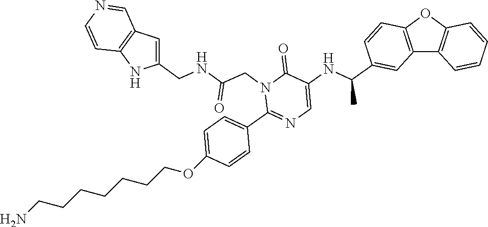 [SMILES: C[C@@H](Nc1cnc(-c2ccc(OCCCCCCCN)cc2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(2-(4- ((7-aminoheptyl)oxy)phenyl)- 5-((1-(dibenzo[b,d]furan-2- yl)ethyl)amino)-6-oxopyrimi- din-1(6H)-yl)acetamide |
| 452 | 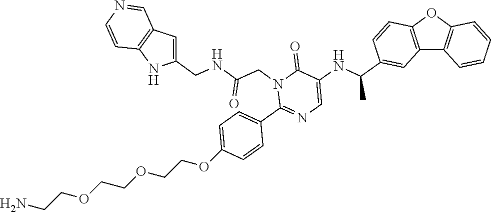 [SMILES: C[C@@H](Nc1cnc(-c2ccc(OCCOCCOCCN)cc2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(2-(4- (2-(2-(2-aminoethoxy)ethoxy)- ethoxy)phenyl)-5-((1-(dibenzo [b,d]furan-2-yl)ethyl)amino)- 6-oxopyrimidin-1(6H)-yl)- acetamide |
| 453 | 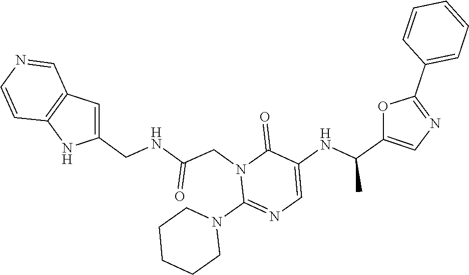 [SMILES: C[C@@H](Nc1cnc(N2CCCCC2)n(CC(=O)NCc2cc3cnccc3[nH]2)c1=O)c1cnc(-c2ccccc2)o1] | (R)-N-((1H-pyrrolo[3,2-c] pyridin-2-yl)methyl)-2-(6-oxo- 5-((1-(2-phenyloxazol-5-yl)- ethyl)amino)-2-(piperidin-1- yl)pyrimidin-1(6H)-yl)acet- amide |
| 454 | 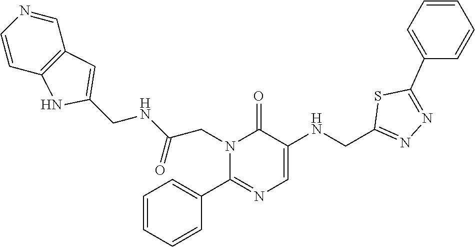 [SMILES: O=C(Cn1c(-c2ccccc2)ncc(NCc2nnc(-c3ccccc3)s2)c1=O)NCc1cc2cnccc2[nH]1] | N-((1H-pyrrolo[3,2-c]pyridin- 2-yl)methyl)-2-(6-oxo-2-phen- yl-5-(((5-phenyl-1,3,4-thiadia- zol-2-yl)methyl)amino)pyr- imidin-1(6H)-yl)acetamide |
| 455 | 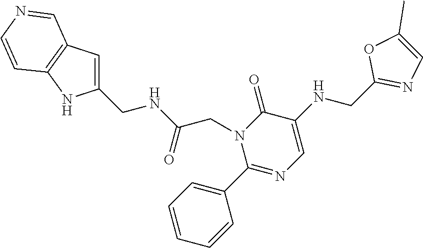 [SMILES: Cc1cnc(CNc2cnc(-c3ccccc3)n(CC(=O)NCc3cc4cnccc4[nH]3)c2=O)o1] | N-((1H-pyrrolo[3,2-c]pyridin- 2-yl)methyl)-2-(5-(((5-methyl- oxazol-2-yl)methyl)amino)- 6-oxo-2-phenylpyrimidin- 1(6H)-yl)acetamide |
| 456 | 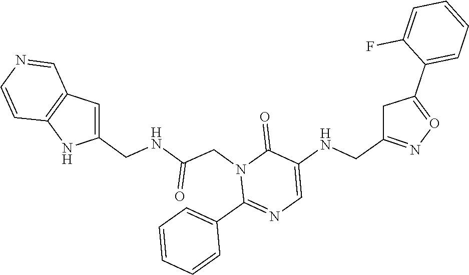 | N-((1H-pyrrolo[3,2-c]pyridin- 2-yl)methyl)-2-(5-(((5-(2- fluorophenyl)isoxazol-3-yl)- methyl)amino)-6-oxo-2-phen- ylpyrimidin-1(6H)-yl)acet- amide |
| 457 | 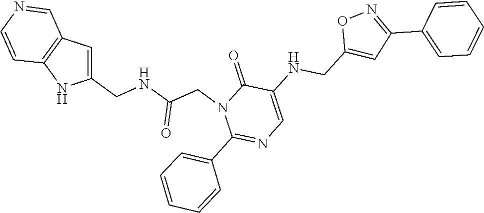 [SMILES: O=C(Cn1c(-c2ccccc2)ncc(NCc2cc(-c3ccccc3)no2)c1=O)NCc1cc2cnccc2[nH]1] | N-((1H-pyrrolo[3,2-c]pyridin- 2-yl)methyl)-2-(6-oxo-2-phen- yl-5-(((3-phenylisoxazol-5- yl)methyl)amino)pyrimidin- 1(6H)-yl)acetamide |
| 458 | 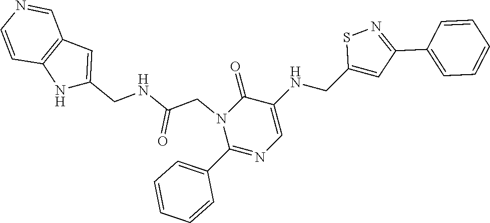 [SMILES: O=C(Cn1c(-c2ccccc2)ncc(NCc2cc(-c3ccccc3)ns2)c1=O)NCc1cc2cnccc2[nH]1] | N-((1H-pyrrolo[3,2-c]pyridin- 2-yl)methyl)-2-(6-oxo-2-phen- yl-5-(((3-phenylisothiazol-5- yl)methyl)amino)pyrimidin- 1(6H)-yl)acetamide |
| 459 | 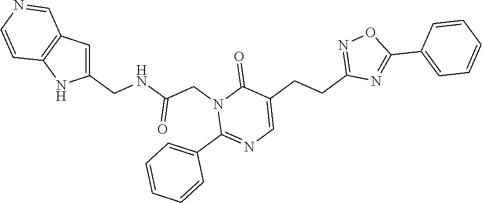 [SMILES: O=C(Cn1c(-c2ccccc2)ncc(CCc2noc(-c3ccccc3)n2)c1=O)NCc1cc2cnccc2[nH]1] | N-((1H-pyrrolo[3,2-c]pyridin- 2-yl)methyl)-2-(6-oxo-2-phen- yl-5-(((5-phenyl-1,2,4-oxadia- zol-3-yl)methyl)amino)pyr- rimidin-1(6H)-yl)acetamide |
| 460 | 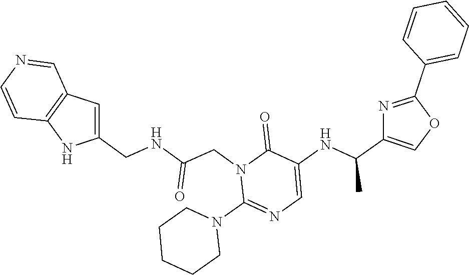 [SMILES: C[C@@H](Nc1cnc(N2CCCCC2)n(CC(=O)NCc2cc3cnccc3[nH]2)c1=O)c1coc(-c2ccccc2)n1] | (R)-N-((1H-pyrrolo[3,2-c] pyridin-2-yl)methyl)-2-(6-oxo- 5-((1-(2-phenyloxazol-4-yl)- ethyl)amino)-2-(piperidin-1- yl)pyrimidin-1(6H)-yl)acet- amide |
| 461 | 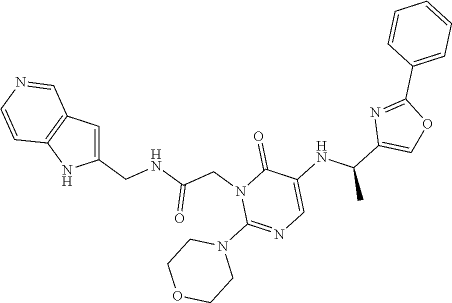 [SMILES: C[C@@H](Nc1cnc(N2CCOCC2)n(CC(=O)NCc2cc3cnccc3[nH]2)c1=O)c1coc(-c2ccccc2)n1] | (R)-N-((1H-pyrrolo[3,2-c] pyridin-2-yl)methyl)-2-(2- morpholino-6-oxo-5-((1-(2- phenyloxazol-4-yl)ethyl)- amino)pyrimidin-1(6H)- yl)acetamide |
| 462 | 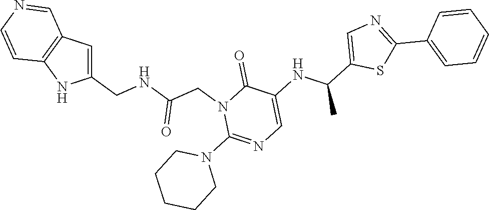 [SMILES: C[C@@H](Nc1cnc(N2CCCCC2)n(CC(=O)NCc2cc3cnccc3[nH]2)c1=O)c1cnc(-c2ccccc2)s1] | (R)-N-((1H-pyrrolo[3,2-c] pyridin-2-yl)methyl)-2-(6- oxo-5-((1-(2-phenylthiazol-5- yl)ethyl)amino)-2-(piperidin- 1-yl)pyrimidin-1(6H)-yl)acet- amide |
| 463 | 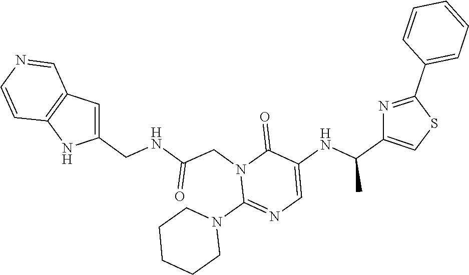 [SMILES: C[C@@H](Nc1cnc(N2CCCCC2)n(CC(=O)NCc2cc3cnccc3[nH]2)c1=O)c1csc(-c2ccccc2)n1] | (R)-N-((1H-pyrrolo[3,2-c] pyridin-2-yl)methyl)-2-(6-oxo- 5-((1-(2-phenylthiazol-4-yl)- ethyl)amino)-2-(piperidin-1-yl)- pyrimidin-1(6H)-yl)acetamide |
| 464 | 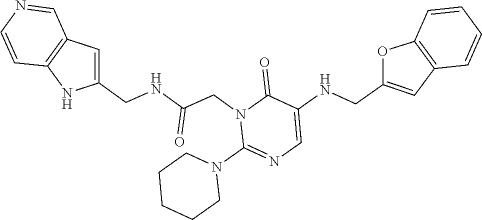 [SMILES: O=C(Cn1c(N2CCCCC2)ncc(NCc2cc3ccccc3o2)c1=O)NCc1cc2cnccc2[nH]1] | N-((1H-pyrrolo[3,2-c]pyridin- 2-yl)methyl)-2-(5-((benzofuran- 2-ylmethyl)amino)-6-oxo-2- phenylpyrimidin-1(6H)-yl)acet- amide |
| 465 | 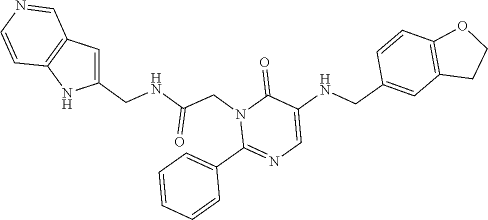 [SMILES: O=C(Cn1c(-c2ccccc2)ncc(NCc2ccc3c(c2)CCO3)c1=O)NCc1cc2cnccc2[nH]1] | N-((1H-pyrrolo[3,2-c]pyridin- 2-yl)methyl)-2-(5-((benzofuran- 5-ylmethyl)amino)-6-oxo-2- phenylpyrimidin-1(6H)-yl)- acetamide |
| 466 | 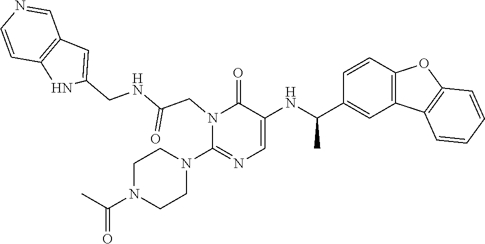 | (R)-N-((1H-pyrrolo[3,2-c] pyridin-2-yl)methyl)-2-(2-(4- acetylpiperazin-1-yl)-5-((1- (dibenzo[b,d]furan-2-yl)ethyl)- amino)-6-oxopyrimidin-1(6H)- yl)acetamide |
| 467 | 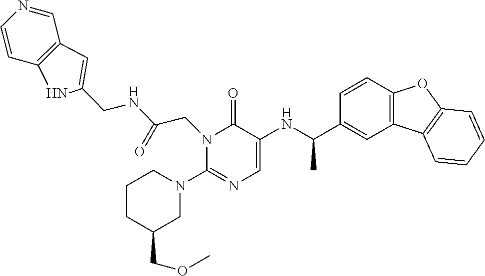 | N-((1H-pyrrolo[3,2-c]pyridin- 2-yl)methyl)-2-(5-(((R)-1- (dibenzo[b,d]furan-2-yl)ethyl)- amino)-2-((S)-3-(methoxy- methyl)piperidin-1-yl)-6-oxo- pyrimidin-1(6H)-yl)acetamide |
| 468 | 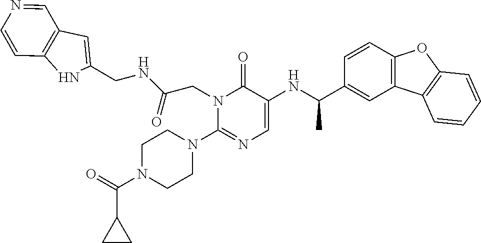 [SMILES: C[C@@H](Nc1cnc(N2CCN(C(=O)C3CC3)CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(2-(4- (cyclopropanecarbonyl)piper- azin-1-yl)-5-((1-(dibenzo[b,d] furan-2-yl)ethyl)amino)-6- oxopyrimidin-1(6H)-yl)acet- amide |
| 469 | 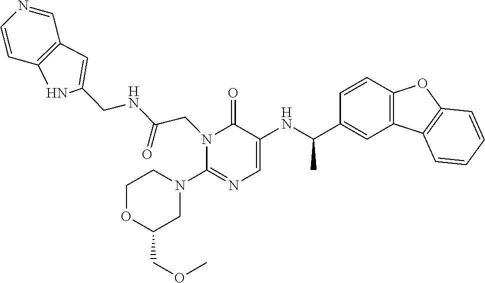 | N-((1H-pyrrolo[3,2-c]pyridin- 2-yl)methyl)-2-(5-(((R)-1- (dibenzo[b,d]furan-2-yl)ethyl)- amino)-2-((R)-2-(methoxy- methyl)morpholino)-6-oxo- pyrimidin-1(6H)-yl)acetamide |
| 470 | 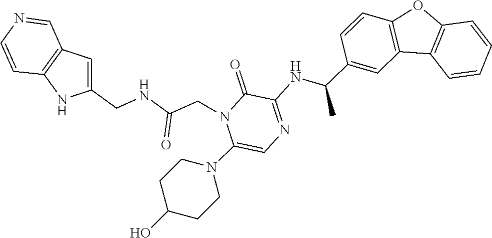 [SMILES: C[C@@H](Nc1ncc(N2CCC(O)CC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-6-(4-hydroxypiperidin- 1-yl)-2-oxopyrazin-1(2H)-yl)- acetamide |
| 471 | 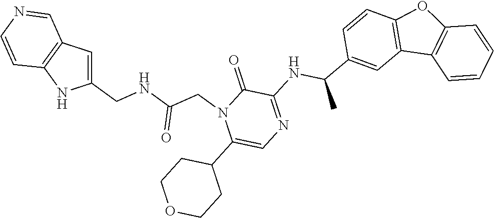 [SMILES: C[C@@H](Nc1ncc(C2CCOCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(3-((1- (dibenzo[b,d]furan-2-yl)ethyl)- amino)-2-oxo-6-(tetrahydro- 2H-pyran-4-yl)pyrazin-1(2H)- yl)acetamide |
| 472 | 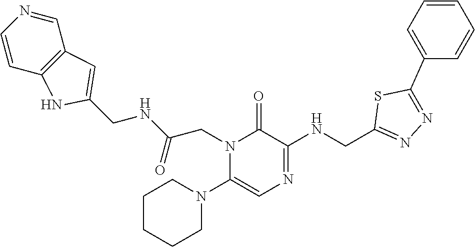 [SMILES: O=C(Cn1c(N2CCCCC2)cnc(NCc2nnc(-c3ccccc3)s2)c1=O)NCc1cc2cnccc2[nH]1] | N-((1H-pyrrolo[3,2-c]pyridin- 2-yl)methyl)-2-(2-oxo-3-(((5- phenyl-1,3,4-thiadiazol-2-yl)- methyl)amino)-6-(piperidin-1- yl) pyrazin-1(2H)-yl)acetamide |
| 473 | 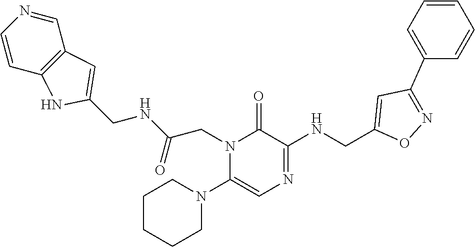 [SMILES: O=C(Cn1c(N2CCCCC2)cnc(NCc2cc(-c3ccccc3)no2)c1=O)NCc1cc2cnccc2[nH]1] | N-((1H-pyrrolo[3,2-c]pyridin- 2-yl)methyl)-2-(2-oxo-3-(((3- phenylisoxazol-5-yl)methyl)- amino)-6-(piperidin-1-yl)pyr- azin-1(2H)-yl)acetamide |
| 474 | 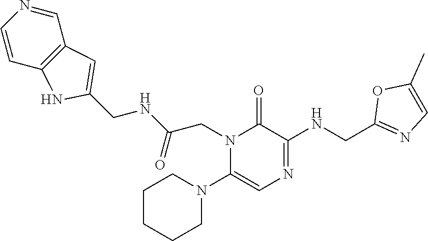 [SMILES: Cc1cnc(CNc2ncc(N3CCCCC3)n(CC(=O)NCc3cc4cnccc4[nH]3)c2=O)o1] | N-((1H-pyrrolo[3,2-c]pyridin- 2-yl)methyl)-2-(3-(((5-methyl- oxazol-2-yl)methyl)amino)- 2-oxo-6-(piperidin-1-yl)pyr- azin-1(2H)-yl)acetamide |
| 475 | 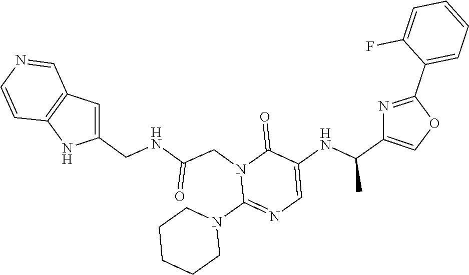 [SMILES: C[C@@H](Nc1cnc(N2CCCCC2)n(CC(=O)NCc2cc3cnccc3[nH]2)c1=O)c1coc(-c2ccccc2F)n1] | (R)-N-((1H-pyrrolo[3,2-c] pyridin-2-yl)methyl)-2-(5-((1- (2-(2-fluorophenyl)oxazol-4- yl)ethyl)amino)-6-oxo-2- (piperidin-1-yl)pyrimidin- 1(6H)-yl)acetamide |
| 476 | 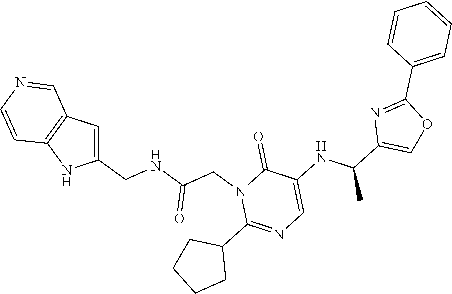 [SMILES: C[C@@H](Nc1cnc(C2CCCC2)n(CC(=O)NCc2cc3cnccc3[nH]2)c1=O)c1coc(-c2ccccc2)n1] | (R)-N-((1H-pyrrolo[3,2-c] pyridin-2-yl)methyl)-2-(2- cyclopentyl-6-oxo-5-((1-(2- phenyloxazol-4-yl)ethyl)- amino)pyrimidin-1(6H)-yl)- acetamide |
| 477 | 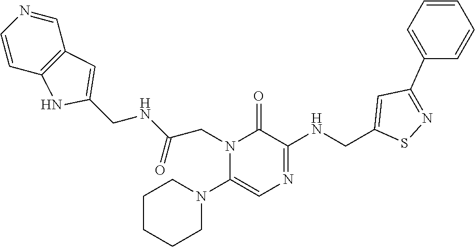 [SMILES: O=C(Cn1c(N2CCCCC2)cnc(NCc2cc(-c3ccccc3)ns2)c1=O)NCc1cc2cnccc2[nH]1] | N-((1H-pyrrolo[3,2-c]pyridin- 2-yl)methyl)-2-(2-oxo-3-(((3- phenylisothiazol-5-yl)methyl)- amino)-6-(piperidin-1-yl)pyr- azin-1(2H)-yl)acetamide |
| 478 | 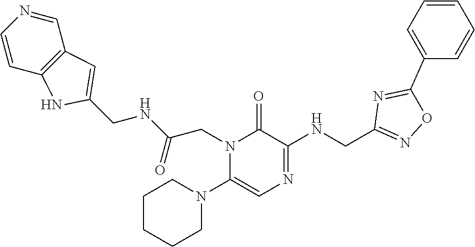 [SMILES: O=C(Cn1c(N2CCCCC2)cnc(NCc2noc(-c3ccccc3)n2)c1=O)NCc1cc2cnccc2[nH]1] | N-((1H-pyrrolo[3,2-c]pyridin- 2-yl)methyl)-2-(2-oxo-3-(((5- phenyl-1,2,4-oxadiazol-3-yl)- methyl)amino)-6-(piperidin-1- yl)pyrazin-1(2H)-yl)acetamide |
| 479 | 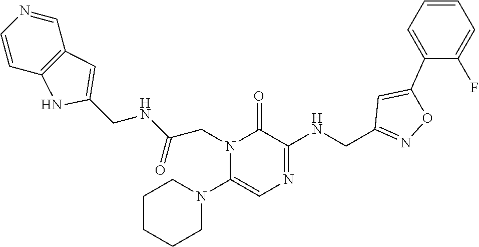 [SMILES: O=C(Cn1c(N2CCCCC2)cnc(NCc2cc(-c3ccccc3F)on2)c1=O)NCc1cc2cnccc2[nH]1] | N-((1H-pyrrolo[3,2-c]pyridin- 2-yl)methyl)-2-(3-(((5-(2- fluorophenyl)isoxazol-3-yl)- methyl)amino)-2-oxo-6-(piper- idin-1-yl)pyrazin-1(2H)-yl)- acetamide |
| 480 | 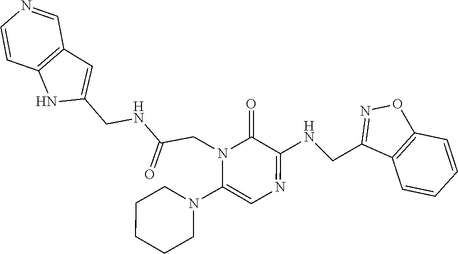 [SMILES: O=C(Cn1c(N2CCCCC2)cnc(NCc2noc3ccccc23)c1=O)NCc1cc2cnccc2[nH]1] | N-((1H-pyrrolo[3,2-c]pyridin- 2-yl)methyl)-2-(3-((benzo[d] isoxazol-3-ylmethyl)amino)-2- oxo-6-(piperidin-1-yl)pyrazin- 1(2H)-yl)acetamide |
| 481 | 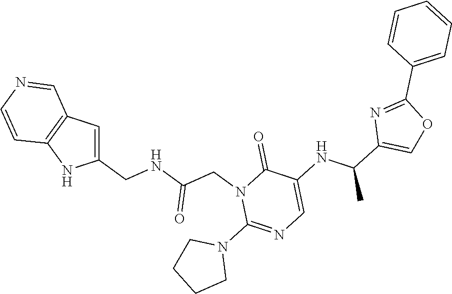 [SMILES: C[C@@H](Nc1cnc(N2CCCC2)n(CC(=O)NCc2cc3cnccc3[nH]2)c1=O)c1coc(-c2ccccc2)n1] | (R)-N-((1H-pyrrolo[3,2-c] pyridin-2-yl)methyl)-2-(6-oxo- 5-((1-(2-phenyloxazol-4-yl)- ethyl)amino)-2-(pyrrolidin-1- yl)pyrimidin-1(6H)-yl)acet- mide |
| 482 | 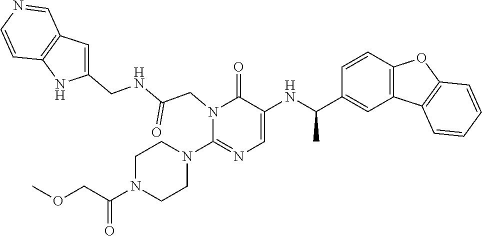 | (R)-N-((1H-pyrrolo[3,2-c] pyridin-2-yl)methyl)-2-(5-((1- (dibenzo[b,d]furan-2-yl)ethyl)- amino)-2-(4-(2-methoxyacet- yl)piperazin-1-yl)-6-oxopyr- imidin-1(6H)-yl)acetamide |
| 483 | 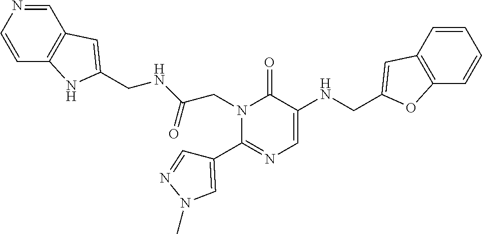 [SMILES: Cn1cc(-c2ncc(NCc3cc4ccccc4o3)c(=O)n2CC(=O)NCc2cc3cnccc3[nH]2)cn1] | N-((1H-pyrrolo[3,2-c]pyridin- 2-yl)methyl)-2-(5-((benzo- furan-2-ylmethyl)amino)-2- (1-methyl-1H-pyrazol-4-yl)- 6-oxopyrimidin-1(6H)-yl)- acetamide |
| 484 | 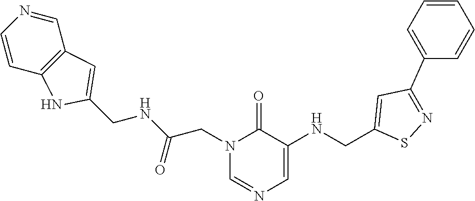 [SMILES: O=C(Cn1cncc(NCc2cc(-c3ccccc3)ns2)c1=O)NCc1cc2cnccc2[nH]1] | N-((1H-pyrrolo[3,2-c]pyridin- 2-yl)methyl)-2-(6-oxo-5-(((3- phenylisothiazol-5-yl)methyl)- amino)pyrimidin-1(6H)-yl)- acetamide |
| 485 | 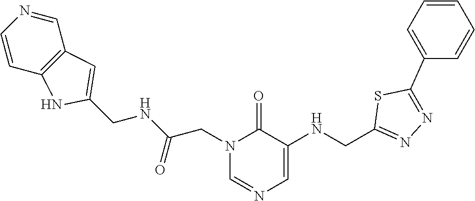 [SMILES: O=C(Cn1cncc(NCc2nnc(-c3ccccc3)s2)c1=O)NCc1cc2cnccc2[nH]1] | N-((1H-pyrrolo[3,2-c]pyridin- 2-yl)methyl)-2-(6-oxo-5-(((5- phenyl-1,3,4-thiadiazol-2-yl)- methyl)amino)pyrimidin- 1(6H)-yl)acetamide |
| 486 | 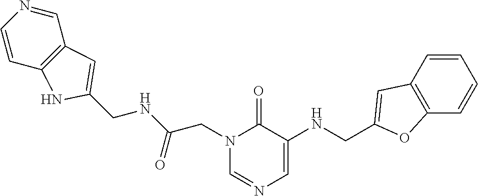 [SMILES: O=C(Cn1cncc(NCc2cc3ccccc3o2)c1=O)NCc1cc2cnccc2[nH]1] | N-((1H-pyrrolo[3,2-c]pyridin- 2-yl)methyl)-2-(5-((benzofuran- 2-ylmethyl)amino)-6-oxopyr- imidin-1(6H)-yl)acetamide |
| 487 | 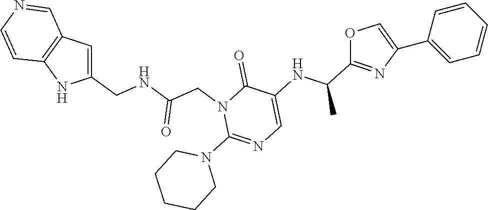 [SMILES: C[C@@H](Nc1cnc(N2CCCCC2)n(CC(=O)NCc2cc3cnccc3[nH]2)c1=O)c1nc(-c2ccccc2)co1] | (R)-N-((1H-pyrrolo[3,2-c] pyridin-2-yl)methyl)-2-(6-oxo- 5-((1-(4-phenyloxazol-2-yl)- ethyl)amino)-2-(piperidin-1-yl)- pyrimidin-1(6H)-yl)acetamide |
| 488 | 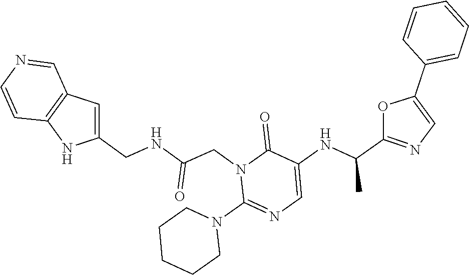 [SMILES: C[C@@H](Nc1cnc(N2CCCCC2)n(CC(=O)NCc2cc3cnccc3[nH]2)c1=O)c1ncc(-c2ccccc2)o1] | (R)-N-((1H-pyrrolo[3,2-c] pyridin-2-yl)methyl)-2-(6-oxo- 5-((1-(5-phenyloxazol-2-yl)- ethyl)amino)-2-(piperidin-1- yl)pyrimidin-1(6H)-yl)acet- amide |
| 489 | 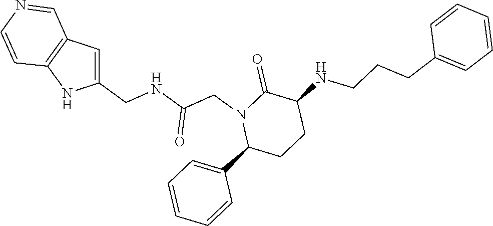 [SMILES: O=C(CN1C(=O)[C@@H](NCCCc2ccccc2)CC[C@H]1c1ccccc1)NCc1cc2cnccc2[nH]1] | N-((1H-pyrrolo[3,2-c]pyridin- 2-yl)methyl)-2-((3S,6S)-2-oxo- 6-phenyl-3-((3-phenylpropyl)- amino)piperidin-1-yl)acetamide |
| 490 | 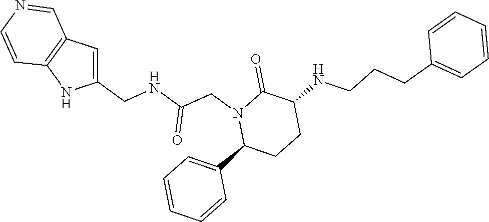 [SMILES: O=C(CN1C(=O)[C@H](NCCCc2ccccc2)CC[C@H]1c1ccccc1)NCc1cc2cnccc2[nH]1] | N-((1H-pyrrolo[3,2-c]pyridin- 2-yl)methyl)-2-((3R,6S)-2-oxo- 6-phenyl-3-((3-phenylpropyl)- amino)piperidin-1-yl)acetamide |
| 491 | 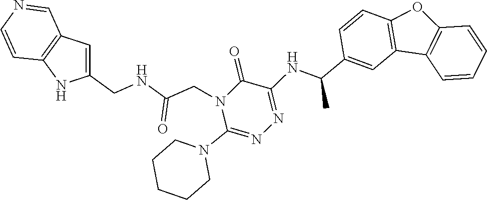 [SMILES: C[C@@H](Nc1nnc(N2CCCCC2)n(CC(=O)NCc2cc3cnccc3[nH]2)c1=O)c1ccc2oc3ccccc3c2c1] | (R)-N-((1H-pyrrolo[3,2-c] pyridin-2-yl)methyl)-2-(6-((1- (dibenzo[b,d]furan-2-yl)ethyl)- amino)-5-oxo-3-(piperidin-1- yl)-1,2,4-triazin-4(5H)-yl)- acetamide |
| 492 | 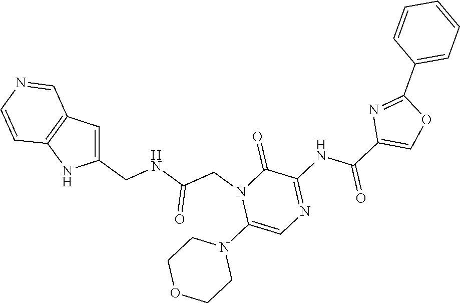 [SMILES: O=C(Cn1c(N2CCOCC2)cnc(NC(=O)c2coc(-c3ccccc3)n2)c1=O)NCc1cc2cnccc2[nH]1] | N-(4-(2-(((1H-pyrrolo[3,2-c] pyridin-2-yl)methyl)amino)- 2-oxoethyl)-5-morpholino-3- oxo-3,4-dihydropyrazin-2-yl)- 2-phenyloxazole-4-carbox- amide |
| 493 | 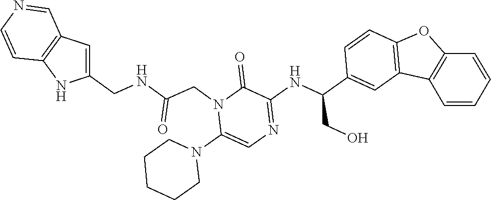 [SMILES: O=C(Cn1c(N2CCCCC2)cnc(N[C@H](CO)c2ccc3oc4ccccc4c3c2)c1=O)NCc1cc2cnccc2[nH]1] | (S)-N-((1H-pyrrolo[3,2-c] pyridin-2-yl)methyl)-2-(3-((1- (dibenzo[b,d]furan-2-yl)-2- hydroxyethyl)amino)-2-oxo- 6-(piperidin-1-yl)pyrazin- 1(2H)-yl)acetamide |
| 494 | 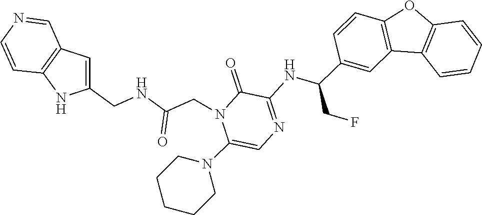 [SMILES: O=C(Cn1c(N2CCCCC2)cnc(N[C@H](CF)c2ccc3oc4ccccc4c3c2)c1=O)NCc1cc2cnccc2[nH]1] | (S)-N-((1H-pyrrolo[3,2-c] pyridin-2-yl)methyl)-2-(3-((1- (dibenzo[b,d]furan-2-yl)-2- fluoroethyl)amino)-2-oxo-6- (piperidin-1-yl)pyrazin-1(2H)- yl)acetamide |
| 495 | 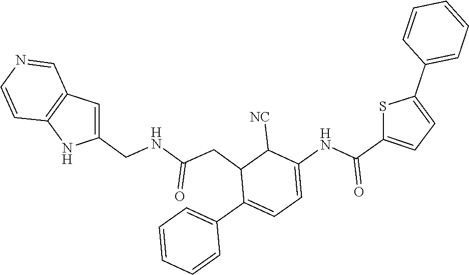 [SMILES: N#CC1C(NC(=O)c2ccc(-c3ccccc3)s2)=CC=C(c2ccccc2)C1CC(=O)NCc1cc2cnccc2[nH]1] | N-(2-(2-(((1H-pyrrolo[3,2-c] pyridin-2-yl)methyl)amino)- 2-oxoethyl)-3-cyano-[1,1′- biphenyl]-4-yl)-5-phenylthio- phene-2-carboxamide |

### Example 3. Human C1s Enzyme Assay

The 1050 values of the compounds of Table 1 were determined with the human C1s enzyme assay described below. Other standard complement assays are also available.

Human complement C1s enzyme (purified from human serum, Complement Technology, Inc.) at 1.16 nM final concentration was incubated with test compound at various concentrations for 5 min at room temperature in 50 mM Tris, 1 M NaCl, pH 7.5. A synthetic substrate Z-L-Lys-SBzl and DTNB (Ellman's reagent) were added to final concentrations of 100 μM each. Absorbance at 405 nm (A405) was recorded at 30 second intervals for 30 md using a microplate spectrophotometer. IC values were calculated by nonlinear regression of complement C1s reaction rates as a function of test compound concentration.

Table 2 shows the IC₅₀ values of the compounds obtained from the above-described human Cis enzyme assay. Three ***s are used to denote compounds with an IC₅₀ less than 100 nanomolar; two ** indicates a compound with an IC₅₀ greater than 100 nanomolar and less than 1 micromolar, one * denotes compounds with an IC₅₀ greater than 1 micromolar. ND indicates a compound for which the IC₅₀ value is not determined.

TABLE 2
C1s Inhibiting Activity of Compounds of the Present Disclosure

| Cmpd | IC₅₀ | Cmpd | IC₅₀ | Cmpd | IC₅₀ | Cmpd | IC₅₀ | Cmpd | IC₅₀ |
|---|---|---|---|---|---|---|---|---|---|
| # | (Stars) | # | (Stars) | # | (Stars) | # | (Stars) | # | (Stars) |
| 1 | *** | 2 | *** | 3 | *** | 4 | *** | 5 | *** |
| 6 | *** | 7 | *** | 8 | *** | 9 | * | 10 | *** |
| 11 | *** | 12 | ** | 13 | ** | 14 | * | 15 | * |
| 16 | * | 17 | *** | 18 | *** | 19 | *** | 20 | *** |
| 21 | *** | 22 | *** | 23 | *** | 24 | *** | 25 | * |
| 26 | *** | 27 | *** | 28 | *** | 29 | *** | 30 | * |
| 31 | * | 32 | *** | 33 | *** | 34 | *** | 35 | *** |
| 36 | *** | 37 | ** | 38 | *** | 39 | *** | 40 | *** |
| 41 | *** | 42 | *** | 43 | * | 44 | ** | 45 | *** |
| 46 | *** | 47 | * | 48 | *** | 49 | *** | 50 | *** |
| 51 | *** | 52 | *** | 53 | *** | 54 | ND | 55 | *** |
| 56 | *** | 57 | *** | 58 | *** | 59 | *** | 60 | *** |
| 61 | *** | 62 | *** | 63 | *** | 64 | *** | 65 | ** |
| 66 | *** | 67 | *** | 68 | *** | 69 | *** | 70 | * |
| 71 | ** | 72 | *** | 73 | *** | 74 | *** | 75 | *** |
| 76 | ** | 77 | *** | 78 | *** | 79 | ** | 80 | * |
| 81 | * | 82 | * | 83 | * | 84 | *** | 85 | * |
| 86 | *** | 87 | ** | 88 | *** | 89 | *** | 90 | *** |
| 91 | *** | 92 | *** | 93 | *** | 94 | *** | 95 | *** |
| 96 | *** | 97 | *** | 98 | *** | 99 | *** | 100 | *** |
| 101 | *** | 102 | *** | 103 | *** | 104 | *** | 105 | *** |
| 106 | *** | 107 | *** | 108 | *** | 109 | *** | 110 | *** |
| 111 | *** | 112 | *** | 113 | *** | 114 | *** | 115 | *** |
| 116 | *** | 117 | *** | 118 | *** | 119 | *** | 120 | *** |
| 121 | ND | 122 | ND | 123 | *** | 124 | *** | 125 | *** |
| 126 | *** | 127 | *** | 128 | ND | 129 | * | 130 | *** |
| 131 | ND | 132 | ND | 133 | ND | 134 | *** | 135 | *** |
| 136 | *** | 137 | ND | 138 | *** | 139 | ND | 140 | ND |
| 141 | *** | 142 | *** | 143 | *** | 144 | * | 145 | ND |
| 146 | ND | 147 | *** | 148 | ND | 149 | ND | 150 | ND |
| 151 | ND | 152 | ND | 153 | *** | 154 | *** | 155 | *** |
| 156 | ND | 157 | ND | 158 | ND | 159 | ND | 160 | ND |
| 161 | ND | 162 | ND | 163 | ND | 164 | *** | 165 | *** |
| 166 | *** | 167 | *** | 168 | *** | 169 | *** | 170 | *** |
| 171 | *** | 172 | *** | 173 | ND | 174 | *** | 175 | *** |
| 176 | *** | 177 | *** | 178 | ND | 179 | *** | 180 | *** |
| 181 | ND | 182 | ND | 183 | ND | 184 | * | 185 | ND |
| 186 | ND | 187 | ND | 188 | ND | 189 | *** | 190 | *** |
| 191 | ND | 192 | *** | 193 | *** | 194 | ND | 195 | ND |
| 196 | ND | 197 | ND | 198 | ND | 199 | ND | 200 | *** |
| 201 | ND | 202 | ND | 203 | *** | 204 | *** | 205 | *** |
| 206 | *** | 207 | * | 208 | *** | 209 | *** | 210 | *** |
| 211 | *** | 212 | *** | 213 | *** | 214 | *** | 215 | *** |
| 216 | *** | 217 | *** | 218 | *** | 219 | *** | 220 | *** |
| 221 | *** | 222 | * | 223 | *** | 224 | ** | 225 | *** |
| 226 | *** | 227 | *** | 228 | *** | 229 | *** | 230 | *** |
| 231 | *** | 232 | *** | 233 | *** | 234 | *** | 235 | * |
| 236 | *** | 237 | *** | 238 | *** | 239 | * | 240 | *** |
| 241 | *** | 242 | *** | 243 | ** | 244 | *** | 245 | *** |
| 246 | *** | 247 | *** | 248 | *** | 249 | *** | 250 | *** |
| 251 | *** | 252 | *** | 253 | *** | 254 | *** | 255 | *** |
| 256 | * | 257 | *** | 258 | *** | 259 | *** | 260 | *** |
| 261 | *** | 262 | *** | 263 | *** | 264 | *** | 265 | *** |
| 266 | * | 267 | * | 268 | * | 269 | * | 270 | *** |
| 271 | *** | 272 | *** | 273 | *** | 274 | *** | 275 | *** |
| 276 | *** | 277 | *** | 278 | * | 279 | *** | 280 | *** |
| 281 | *** | 282 | *** | 283 | *** | 284 | *** | 285 | *** |
| 286 | *** | 287 | *** | 288 | *** | 289 | *** | 290 | ** |
| 291 | *** | 292 | *** | 293 | *** | 294 | ** | 295 | *** |
| 296 | *** | 297 | *** | 298 | *** | 299 | *** | 300 | *** |
| 301 | *** | 302 | *** | 303 | *** | 304 | ** | 305 | ** |
| 306 | ** | 307 | *** | 308 | *** | 309 | *** | 310 | ** |
| 311 | ** | 312 | *** | 313 | ** | 314 | *** | 315 | *** |
| 316 | *** | 317 | *** | 318 | *** | 319 | * | 320 | *** |
| 321 | *** | 322 | *** | 323 | *** | 324 | ** | 325 | *** |
| 326 | *** | 327 | *** | 328 | *** | 329 | ** | 330 | *** |
| 331 | * | 332 | * | 333 | *** | 334 | *** | 335 | *** |
| 336 | *** | 337 | *** | 338 | ** | 339 | *** | 340 | ** |
| 341 | ** | 342 | ** | 343 | ** | 344 | *** | 345 | *** |
| 346 | ** | 347 | *** | 348 | *** | 349 | *** | 350 | *** |
| 351 | ** | 352 | ** | 353 | *** | 354 | *** | 355 | ** |
| 356 | * | 357 | ** | 358 | *** | 359 | *** | 360 | *** |
| 361 | *** | 362 | ** | 363 | * | 364 | * | 365 | ** |
| 366 | * | 367 | * | 368 | * | 369 | ** | 370 | ** |
| 371 | * | 372 | *** | 373 | * | 374 | * | 375 | *** |
| 376 | *** | 377 | *** | 378 | * | 379 | * | 380 | ** |
| 381 | * | 382 | *** | 383 | * | 384 | ** | 385 | * |
| 386 | * | 387 | ** | 388 | ** | 389 | *** | 390 | *** |
| 391 | ** | 392 | *** | 393 | ** | 394 | *** | 395 | * |
| 396 | *** | 397 | *** | 398 | *** | 399 | *** | 400 | *** |
| 401 | ** | 402 | ** | 403 | *** | 404 | * | 405 | *** |
| 406 | ** | 407 | *** | 408 | *** | 409 | *** | 410 | *** |
| 411 | *** | 412 | *** | 413 | *** | 414 | *** | 415 | *** |
| 416 | *** | 417 | *** | 418 | *** | 419 | ND | 420 | *** |
| 421 | *** | 422 | *** | 423 | *** | 424 | *** | 425 | * |
| 426 | *** | 427 | *** | 428 | *** | 429 | *** | 430 | *** |
| 431 | *** | 432 | *** | 433 | *** | 434 | *** | 435 | *** |
| 436 | *** | 437 | *** | 438 | *** | 439 | *** | 440 | ** |
| 441 | *** | 442 | *** | 443 | *** | 444 | *** | 445 | *** |
| 446 | *** | 447 | *** | 448 | ** | 449 | *** | 450 | *** |
| 451 | *** | 452 | *** | 453 | *** | 454 | *** | 455 | *** |
| 456 | *** | 457 | *** | 458 | *** | 459 | *** | 460 | ** |
| 461 | *** | 462 | *** | 463 | ** | 464 | *** | 465 | *** |
| 466 | *** | 467 | * | 468 | ** | 469 | ** | 470 | *** |
| 471 | *** | 472 | *** | 473 | *** | 474 | *** | 475 | ** |
| 476 | ** | 477 | *** | 478 | *** | 479 | *** | 480 | *** |
| 481 | ** | 482 | *** | 483 | *** | 484 | *** | 485 | *** |
| 486 | *** | 487 | *** | 488 | *** | 489 | * | 490 | * |
| 491 | *** | 492 | *** | 493 | * | 494 | * | 495 | ND |

### Example 4. Hemolysis Assay

The hemolysis assay was previously described by Dodds, A. W. and Sim, R. B. (1997); Morgan, B. P. (2000). Prior to the assay, the optimum concentration of Normal Human Serum (NHS) needed to achieve 100% lysis of antibody sensitized sheep erythrocytes (EA) is determined by titration. EA are sheep erythrocytes with rabbit IgM anti-sheep erythrocyte antibodies bound to their surface. In the assay, NHS (Complement Technology) is diluted in GVB++ Buffer (0.1% gelatin, 5 mM Veronal, 145 mM NaCl, 0.025% NaN₃, pH 7.3, 0.15 mM calcium chloride and 0.5 mM magnesium chloride, Complement Technology) and incubated with test compound at various concentrations for 2 min at room temperature. EA (Complement Technology) freshly suspended in GVB++ are added to a final concentration of 1×10⁸ cells/mL and reactions are incubated for 60 min at 37° C. Positive control reactions (100% lysis) consist of GVB++ with NHS and EA but without test compound; negative control reactions (0% lysis) consist of GVB++ with EA only. Samples are centrifuged at 2000 g for 3 min and supernatants collected. Absorbance at 405 nm (A405) is recorded using a microplate spectrophotometer. IC₅₀ values are calculated by nonlinear regression from the percentage of hemolysis as a function of test compound concentration.

### Example 5. Microsomal Stability Assay

The microsomal stability of compounds of the disclosure are analyzed as described below.

### Materials

| Chemicals and Reagents: | Product Number |
|---|---|
| Deionized Water [H₂O] | |
| Acetonitrile [ACN] (HPLC Grade) | Sigma Aldrich #34998 |
| Dimethyl Sulfoxide [DMSO] | Sigma Aldrich #D8418 |
| Wistar-Han Rat Liver Microsomes [RLM] | XenoTech #R6000 |
| Beagle Dog Liver Microsomes [DLM] | XenoTech #D1000 |
| Cynomolgus Monkey Liver Microsomes [MLM] | XenoTech #P2000 |
| Human Liver Microsomes [HLM] | XenoTech #H2610 |
| β-Nicotinamide adenine dinucleotide | Sigma Aldrich #N3886 |
| phosphate sodium salt [NADP+] | |
| Glucose-6-phosphate | Sigma Aldrich #G7879 |
| Glucose-6-phosphate dehydrogenase | Sigma Aldrich #G6378 |
| Magnesium Chloride [MgCl₂] | Sigma Aldrich #M8266 |
| Phosphate Buffer [0.5 mM] | Corning Life Sciences #451201 |

### Methodology

The Phosphate Buffer solution is prepared by mixing 15.2 mL of deionized water with 1.9 mL of 0.5 M Phosphate Buffer and 1.9 mL of 33 mM MgCl₂. The Microsomal Working solution is prepared by mixing 14.655 mL of deionized water with 1.9 mL of 0.5 mM Phosphate Buffer, 1.9 mL of 33 mM MgCl₂, and 545 μL of 20 mg/mL of liver microsomes (species specific). The Cofactor Solution is prepared by mixing 8.12 mL of deionized water with 1.45 mL of 0.5 mM Phosphate Buffer, 1.45 mL of 33 mM MgCl₂, 1.16 mL of 100 mM NADP+ solution, 1.16 mL of 100 mM Glucose-6-Phosphate solution, and 1.16 mL of 100 U/mL Glucose-6-Phosphate Dehydrogenase solution. Final assay reagent concentrations are 0.1 μM Test Article, 0.5 mg/mL liver microsomes, 1 mM NADP+, 5 mM Glucose-6-Phosphate, 1 U/mL Glucose-6-phosphate dehydrogenase, 50 mM Potassium Phosphate Buffer (pH 7.4), 3.3 mM MgCl₂, and 0.2% DMSO.

The assay reaction mixture is prepared by taking 436 μL of Microsomal Working Solution and mixing it with 63 μL of Cofactor Solution in a 1.5 mL 96-well plate and preincubated at 37° C. for 5 minutes. The assay was initiated by the addition of 1 μL of 5 0 μM solution of a test compound in DMSO.

At 0 minutes, 30 minutes, and 120 minutes, 100 μL aliquots of the incubation mixture are removed and mixed with 200 μL of cold acetonitrile containing 0.2 μM Tolbutamide (internal standard) in a separate 96-well plate. At the conclusion of the assay, the sample collection plate are vortexed for thirty seconds, and centrifuged at 3000×g and 4° C. for 10 minutes. The supernatants were transferred to clean 96-well bioanalysis plates for analysis and stored at 4° C.

### Sample Bioanalysis

The test compound concentrations in liver microsome samples are analyzed by a LC-MS/MS method on a Sciex API 5500 Q-Trap mass spectrometer using turbo ion spray with MRM monitoring in the positive mode. Analyst® software (Version 1.6.2) was used to capture the LC-MS/MS data and integrate the peak areas.

### Example 6. Hepatic Clearance Assay

The hepatic clearance of compounds of the present disclosure are analyzed as described below.

### Thawing Media and Test Article Preparation

The thawing media is warmed in a water bath 37° C. The test compounds are diluted 200× from 20 mM to 100 μM in DMSO, then to 0.2 μM by adding 3 μL of test compound solution into 1.5 mL of Williams E medium. Midazolam (1 mM) and 7-ethoxycoumarin (100 mM) controls are thawed, and 3 μL of each control is added to 1.5 mL Williams E medium. Cold acetonitrile (150 μL) with internal standards are then added to a 96-well plate.

### Cell Viability Determination

Cryopreserved hepatocytes (human, rat, dog, or monkey) are thawed in water bath for ˜90 s, transferred into warm thawing media, and centrifuged at 100 g for 5 min. The supernatant fluid is then aspirated, and the pellets are resuspended in 5 mL Williams E medium. The cell suspension (50 μL) is mixed with Trypan blue, then an aliquot is placed on a hemacytometer for cell viability determination. If the cell viability is determined to be over 70%, the cell concentration in the hepatocyte medium is then adjusted to 10⁶/mL by adding the appropriate amount of Williams E medium.

### Incubation

The hepatocyte suspension (500 μL) is added to each well of 24-well plate. The test compound solution (500 μL) is then added. At 0 min, 75 μL of the sample is taken and quenched with 150 μL cold acetonitrile in the 96-well plate. Samples are taken and quenched at 30 min, 50 min, and 120 min (15 min and 120 min for 7-ethoxycoumarin). After incubation and quenching, the samples are stored at −20° C. overnight.

### LC-MS/MS Analysis

After the mass spectrometer is tuned for each compound using a 2 mL solution of 0.05 MS tuning for each compound 0.05 μM of the test compound in 1:1 Water/Acetonitrile, the 96-well plate containing the incubation samples and quench solutions is centrifuged at 25° C., 3000 rpm for 15 min. The samples (160 μL) are transferred to deep well pates and queued in LC-MS/MS for analysis. To determine the Intrinsic clearance and hepatic clearance (% Qh) of the test compounds, the in peak area ratio (compound peak area/internal standard peak area) is plotted against time and the gradient of the line determined.

### Example 7. Caco-2 Permeability Assay

The bidirectional permeability and efflux probability of compounds of the present disclosure in Caco-2 cell monolayers with and without efflux inhibitors are determined following the procedure described below.

### Materials and Reagents

Caco-2 cells are obtained from the American type culture collection (ATCC), and the ATCC® Number is HTB-37. Hepes, Penicillin, Streptomycin, Trypsin/EDTA and DMSO are purchased from Solarbio. Bovine Serum Albumin (BSA) is purchased from Solarbio Biotechnology Co., Ltd. Fetal bovine serum, Hank's balanced salt solution (HBSS) and Non-essential amino acids (NEAA) are all purchased from Gibco by Thermo Fisher Scientific. Dulbecco's Modified Eagle's Medium (DMEM) is purchased from Corning Corporation or Hyclone. HTS Transwell-96 Well (Cat. No. 3391) Permeable Supports and other sterile plastic ware are purchased from Corning Corporation. Millicell Epithelial Volt-Ohm measuring system is purchased from Millipore. Cellometer® Vision is purchased from Nexcelom Bioscience LLC. Infinite 200 PRO microplate reader is purchased from Tecan. MTS2/4 orbital shaker is purchased from IKA Labortechnik.

Transport buffer: HBSS containing 25 mM HEPES, pH 7.4 and 3% BSA: to prepare the HBSS containing 25 mM HEPES, pH 7.4, accurately weigh 5.958 g of HEPES and 0.35 g sodium hydrogen carbonate and add into 900 mL of pure water, then sonicate to dissolve the content. Transfer 100 mL of 10×HBSS into the solution, and place the solution on a stirrer, slowly adjust pH with sodium hydrate to 7.4, following with filtering and add 3% BSA.

Transport buffer with efflux inhibitors: HBSS containing 25 mM HEPES, pH 7.4 and 3% BSA: to prepare the HBSS containing 25 mM HEPES, pH 7.4, accurately weigh 5.958 g of HEPES and 0.35 g sodium hydrogen carbonate and add into 900 mL of pure water, then sonicate to dissolve the content. Transfer 100 mL of 10×HBSS into the solution, and place the solution on a stirrer, slowly adjust pH with sodium hydrate to 7.4, following with filtering. Before the experiment, add the 10 mM zosuquidar, 30 mM benzbromarone and 2 mM KO-143 into the HBSS (25 mM HEPES, pH 7.4) with 1000-fold dilution and 3% BSA at a final concentration of 1 μM zosuquidar, 30 μM benzbromarone, and 2 μM KO-143.

### Preparation for Cell Seeding

1. Prepare transport buffer (HBSS containing 25 mM HEPES, pH 7.4): accurately weigh 5.958 g of HEPES and 0.35 g sodium hydrogen carbonate and add into 900 mL of pure water, then sonicate to dissolve the content. Transfer 100 mL of 10×HBSS into the solution, and place the solution on a stirrer, slowly adjust pH with sodium hydroxide to 7.4, following with filtering.
2. Prepare Caco-2 cell culture medium consisting of Dulbecco's Modified Eagle's Medium (DMEM) with high glucose and L-glutamine supplemented with 10% FBS, 0.1 mg/mL of streptomycin, 100 units of penicillin, 0.6 μg/mL of Kanamycin sulfate and 1× non-essential amino acids (NEAA).
3. Add 50 μL of culture medium to each well of the Transwell insert. Remove Transwell insert from reservoir and add 25 mL of culture medium.
4. Incubate at 37° C., 5% CO₂ for 1 hour. Plates are ready for cell seeding.
5. Cultivate cells in T-75 flasks in a cell culture incubator set at 37° C., 5% CO₂, 95% relative humidity. Allow cells to reach 80-90% confluence before detaching and splitting.
6. Rinse cultivated cells in T-75 flasks with 5 mL PBS. Aspirate off, add 1.5 mL trypsin/EDTA, and incubate at 37° C. for approximately 5 to 10 minutes or until the cells detach and float. Inactivate trypsin/EDTA by adding excess medium containing serum.
7. Transfer cell suspension to a conical tube and pellet cells by centrifugation at 120×g for 10 minutes.
8. Resuspend cells in seeding medium at a density of 6.86×10⁵ cells/mL. This cell concentration can be used to seed 2.4×10⁵ cells/cm².
   Seeding and Feeding of Caco-2 Cells into Transwell Plates
1. Add 50 μL of above cell suspension to each well of a previously prepared Transwell plate.
2. Incubate the plate for 14-18 days. Replace the medium every other day, beginning no sooner than 48 hours after initial plating. Medium must be replaced on the day before conducting the experiment.
3. The procedure for medium changes is as follows. Remove the plate from incubator and place in hood. Aspirate the medium from reservoir and each Transwell insert. Add 100 μL of culture medium to each well of Transwell inserts and 25 mL of culture medium to reservoir tray. Return plate to incubator.

### Assessment of Cell Monolayer Integrity

1. When the 14-day cultured Caco-2 cells have reached confluence and are differentiated, they are ready to be used for transport studies.
2. Measure the electrical resistance across the monolayer using Millicell Epithelial Volt-Ohm measuring system (Millipore).
3. Record the electrical resistance for each well.
4. Once all wells have been measured, return plate to incubator.
5. Calculation of TEER values: TEER measurement (ohms)×Area of membrane (cm²)=TEER value (ohm-cm²). Any monolayer with a TEER value <230 ohms-cm², indicating poor monolayer formation, will be discarded.

### Performing the Drug Transport Assay

1. Remove the Caco-2 plate from the incubator. Next, wash the monolayer, exchange the volume two times using pre-warmed transport buffer. Then, incubate the plate at 37° C. for 30 minutes.
2. For test compounds and control compound digoxin, prepare stock solutions in DMSO at 2 mM and dilute by transport buffer with or without efflux Inhibitors to get 10 μM working solution. For control compound metoprolol, prepare stock solutions in DMSO at 2 mM and dilute by HBSS without 3% BSA to get 10 μM working solution. The final DMSO concentration is 0.8%.
3. Prepare bulk donor solutions with 597 μL transport buffer (with or without efflux inhibitors) and 3 μL 2 mM test compound or control compound.
4. Prepare bulk receiver solutions with 597 μL transport buffer (with or without efflux inhibitors) and 3 μL DMSO.
5. To determine the rate of drug transport in the apical to basolateral direction. Add 108 μL of the donor solution to the Transwell insert (apical compartment), and transfer 8 μL sample immediately from the apical compartment to 72 μL transport buffer and 240 μL quenching solvents in a new 96-well plate as the initial donor sample (A-B). Shake the plate at 1000 rpm for 5 minutes. Fill the wells in the receiver plate (basolateral compartment) with 300 μL of receiver solution.
6. To determine the rate of drug transport in the basolateral to apical direction. Add 308 μL of the donor solution to the receiver plate wells (basolateral compartment), and transfer 8 μL sample immediately from the basolateral compartment to 72 μL transport buffer and 240 μL quenching solvents in a new 96-well plate as the initial donor sample (B-A). Fill the Transwell insert (apical compartment) with 100 μL of receiver solution. Shake the plate at 1000 rpm 5 minutes.
7. Incubate at 37° C. for 2 hours. At the end of the transport period, transfer 8 μL of samples from donor sides (apical compartment for Ap→BI flux, and basolateral compartment for BI→Ap flux) to 72 μL transport buffer and 240 μL quenching solvents in a new 96-well plate. Remove 80 μL directly from receiver sides (basolateral compartment for Ap→BI flux, and apical compartment for BI→Ap flux) and transfer to new 96-well plates with 240 μL quenching solvents. Vortex at 1000 rpm for 10 minutes. Centrifuge the quenching plates of each time point for 30 minutes at 4,000 rpm and 4° C. Transfer 100 μL of supernatant of each compound into a 96-well analysis plate containing 100 μL of pure water in the corresponding wells. Shake the analysis plate at 1,000 rpm for 2 minutes and the sample is analyzed by LC-MS/MS. All incubations are performed in duplicate.
8. To determine the Lucifer yellow leakage after 2-hour transport period, prepare stock solutions of Lucifer yellow in water and dilute with transport buffer to reach the final concentration of 100 μM. Add 100 μL of the Lucifer yellow solution to the Transwell insert (apical compartment). Fill the wells in the receiver plate (basolateral compartment) with 300 μL of transport buffer. Incubate at 37° C. for 30 minutes. Remove 80 μL directly from the apical and basolateral wells (using the basolateral access holes) and transfer to new 96 wells plates. Measure Lucifer Yellow fluorescence (to monitor monolayer integrity) in a fluorescence plate reader at 485 nm excitation and 530 nm emission.

### Example 8. Pharmacokinetic Profile following IV and PO Administration in Rats

The pharmacokinetic profile of compounds of the present disclosure following intravenous (IV) or oral (PO) administration in male Wistar Han rats at various dosages is assessed based on the general procedure described below.

### Exemplary Dosing Information

### IV Dosing:

For Male Wistar Han rats previously fitted with a jugular vein catheter (JVC) for blood collection, the animals will receive a single IV dose of a test compound (e.g., at a dose level of 0.5 mg/kg or more). Sampling will be done from the cannulated carotid artery.

### PO Dosing:

For Male Wistar Han rats previously fitted with a jugular vein catheter for blood collection animals will receive a single oral dose of compound, e.g., at a dose level of 5 mg/kg or more.

### Animal Feeding Control:

All animals for IV and PO administration will be fasted overnight and fed after 4 hours collection post dosing.

### Dose Formulation Processing During Dosing:

The dose formulations will be kept at room temperature and administered within 2 hours after preparation.

### Exemplary Pharmacokinetic (PK) Schedule:

### IV Dosing:

Plasma: Pre-dose, 0.33, 0.083, 0.25, 0.5, 1, 2, 4, 8, and 24 hours pose dose.

### PO Dosing:

Plasma: Pre-dose, 0.25, 0.5, 1, 2, 4, 8, and 24 hours post dose.

### PK Samples Analyses

Concentrations of test compounds in the plasma samples will be analyzed using a LC-MS/MS method. WinNonlin (Phoenix™, version 8.3) or other a similar software will be used for pharmacokinetic calculations. The following pharmacokinetic parameters will be calculated, whenever possible, from the plasma concentrations versus time data:
IV administration: T_{1/2}, C₀, AUCₗₐₛₜ, AUC_{inf}, MRT, Cl, Vss
PO administration: T_{1/2}, Cₘₐₓ, Tₘₐₓ, AUC_{inf}, AUCₗₐₛₜ, F %

### Example 9. MASP-2 Protease Assay

Compounds of the present disclosure are assessed with a MASP-2 protease assay.

### Materials

| | |
|---|---|
| Buffer | 20 mM Hepes, 140 mM NaCl, pH 7.4 |
| Enzyme | MASP-2 (CCP2-SP) (Charles River) |
| Substrate | Z-Lys-SBzl (Sigma Aldrich) |
| Detection | DTNB Ellman's Reagent (Sigma Aldrich) |

### Method

The test compounds and/or DMSO are preincubated with enzyme in buffer for 5 minutes at Room temperature (RT), and also without enzyme as a control. The reaction is initiated by addition of substrate and DTNB mix. In this assay, the concentrations of the enzyme, substrate, and DTNB concentration are 16 nM, 200 μM and 200 μM, respectively. The absorbance is measured on a microplate reader with 405 nm (A₄₀₅) in a kinetic mode every 30 second for at least 30 minutes at RT. The test compounds are screened at a concentration of 0.1 μM or in an 8 point half log dilution series with a starting concentration of 10 μM. The compound, (2R,4S)—N—((S)-1-(((1H-pyrrolo[3,2-c]pyridin-2-yl)methyl)amino)-1-oxopropan-2-yl)-4-phenylpiperidine-2-carboxamide is included in each assay in an 8 point half log dilution series with a starting concentration of 1 μM.

### OTHER EMBODIMENTS

This specification has been described with reference to various specific embodiments. However, one of ordinary skill in the art appreciates that various modifications and changes can be made without departing from the scope of the invention as set forth in the claims. Accordingly, the specification is to be regarded in an illustrative rather than a restrictive sense, and all such modifications are intended to be included within the scope of the claims.

We claim:

1. A compound of formula (I): 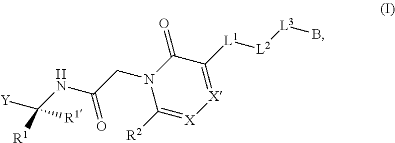
or a pharmaceutically acceptable salt thereof,
wherein
each of R¹ and R^{1′} is independently H or optionally substituted C₁-C₆ alkyl;
X is CR³ or N;
X is CR⁴ or N;
R² is H, C₁-C₆ alkyl, optionally substituted C₆-C₁₄ aryl, optionally substituted C₃-C₈ carbocyclyl, optionally substituted 5- to 10-membered heteroaryl, optionally substituted 4- to 10-membered heterocyclyl, or optionally substituted (4- to 10-membered heterocyclyl)oxy, or optionally substituted (5- to 9-membered heteroaryl)oxy;
each of R³ and R⁴ is independently H, halo, or optionally substituted C₁-C₆ alkyl;
L¹ is a bond; NH; NHC(O); NHC(O)O; NHC(O)NH, or NHS(O)₂;
L² is a bond or optionally substituted C₁-C₆ alkylene;
L³ is a bond, NH, NHC(O), C(O), O, S(O)₂CH₂;
B is halo, optionally substituted C₆-C₁₄ aryl, optionally substituted C₃-C₁₄ carbocyclyl, optionally substituted 5- to 14-membered heterocyclyl; or optionally substituted 5- to 10-membered heteroaryl; and
Y is 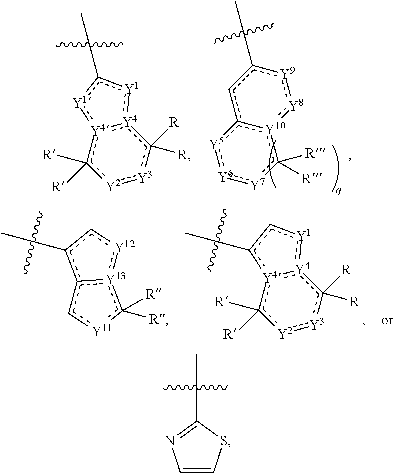
wherein
Y¹ is O, S, NR^{d}, wherein each R^{d} is independently absent, H, or C₁-C₆ alkyl;
Y¹ is O, S, NR^{d}, or C(R^{d})₂;
each of Y² and Y³ is independently NR^{e} or C(R^{e})₂, wherein each R^{e} is independently absent; H; optionally substituted C₁-C₆ alkyl; halo; or N(R^{g})₂, wherein each R^{g} is independently H or C₁-C₆ alkyl; or both R^{e} combine to form oxo;
each of Y⁴, Y^{4′}, Y¹⁰, and Y¹³ is independently CR^{e} or N;
each of Y⁵, Y⁶, and Y⁷ is independently O, S, NR^{f} or C(R¹)₂, wherein each R^{f} is independently absent; H; optionally substituted C₁-C₆ alkyl; halo; or N(R^{g})₂; or both R^{f} combine to form oxo;
each of Y⁸ and Y⁹ is independently C(R¹)₂ or NR^{f};
each of Y¹¹ and Y¹² is independently NR^{e}, C(R^{e})₂, S, or O;
each [symbol] is independently a single bond or a double bond;
each of R, R′, R″, and R′″ is independently absent, H, optionally substituted C₁-C₆ alkyl, halo, or N(R^{g})₂; or
both R combine to form oxo; or
both R′ combine to form oxo; and
q is 0 or 1;
provided that at least one of the following is true:
(i) -L¹-L²-L³- do not combine to form 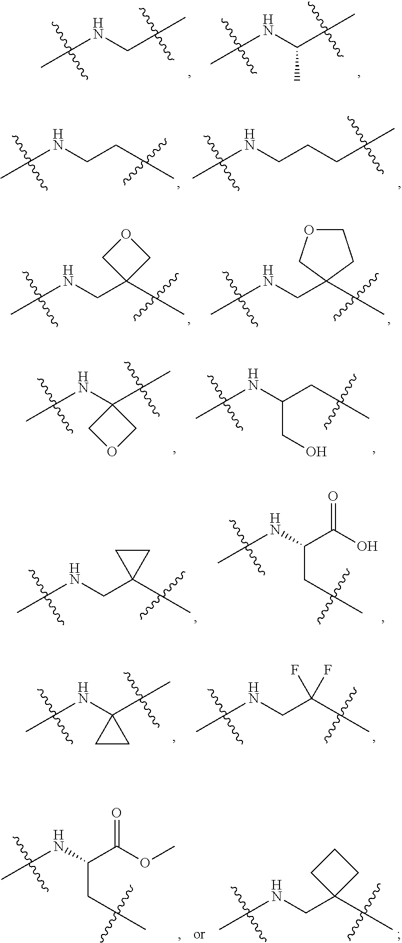
(ii) R² is not C₁-C₆ alkyl, 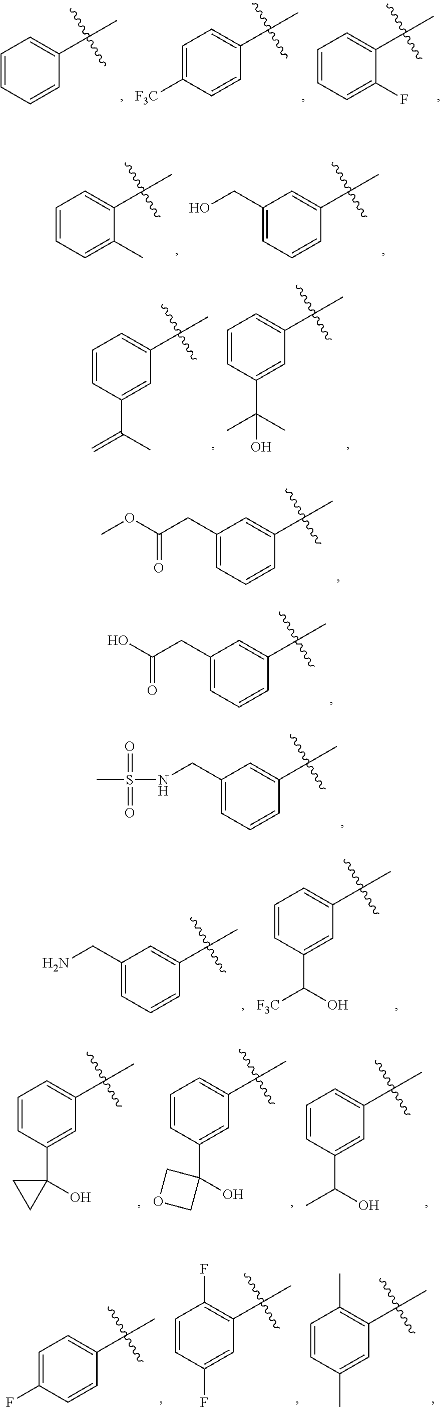 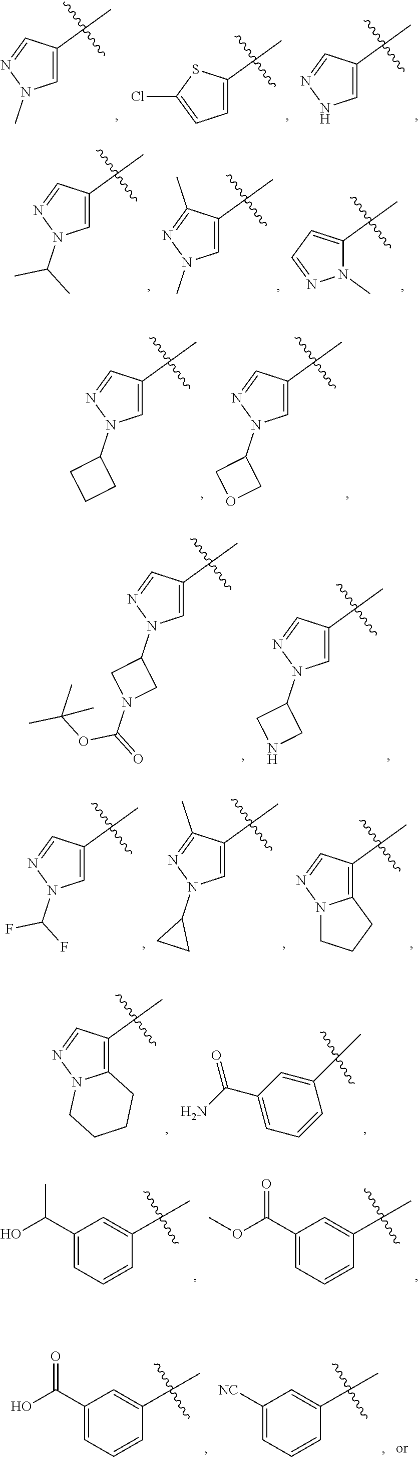 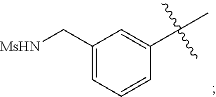
(iii) B is not 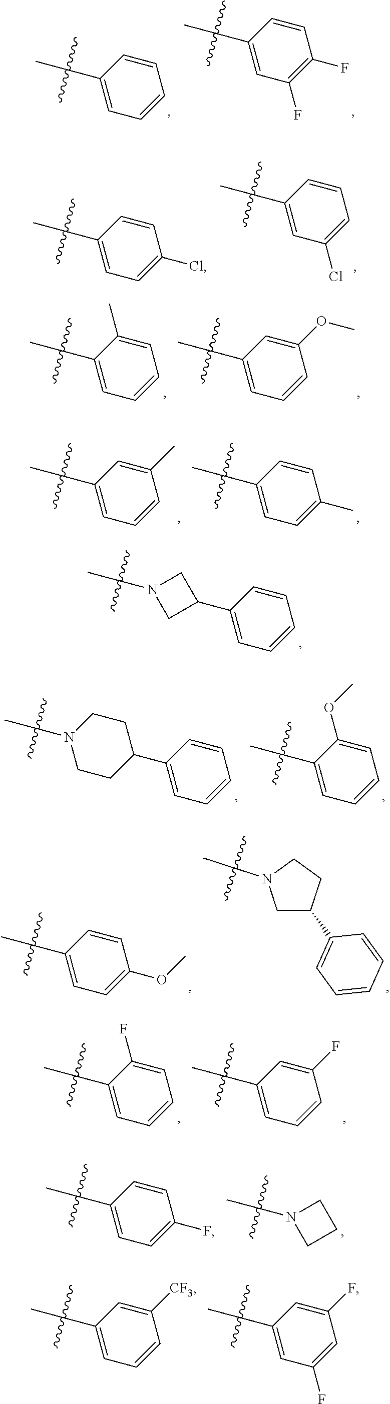 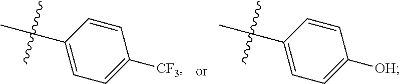 [SMILES: *c1ccc(C(F)(F)F)cc1 ; *c1ccc(O)cc1]
(iv) at least one of R¹ and R^{1′} is not H;
(v) X is N;
(vi) X is CR⁴, and
(vii) Y is not

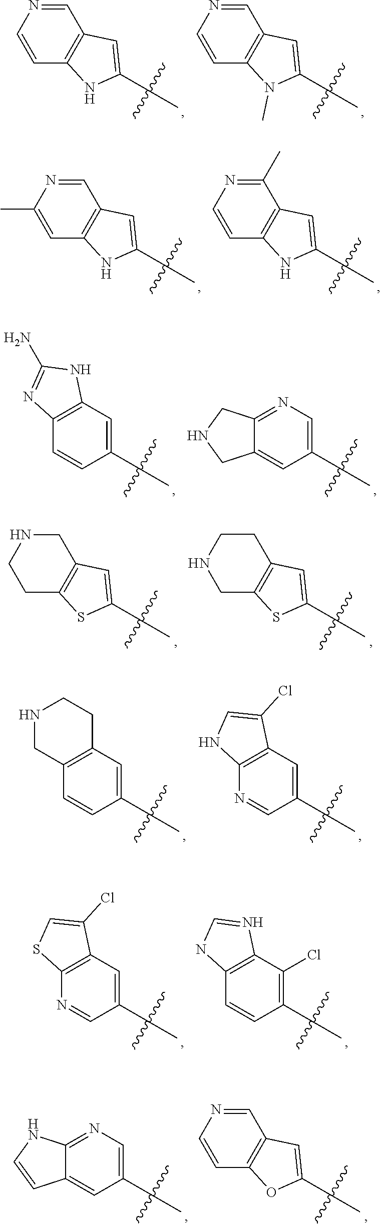

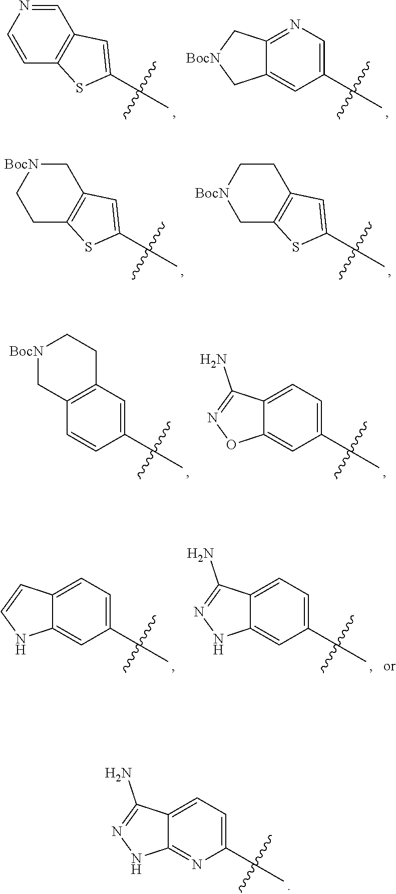

2. The compound of claim 1, wherein:
R¹ is H; and/or
R¹ is H or methyl; and/or
X is CH, CCH₃, or N; and/or
X′ is CH or N.

3-9. (canceled)

10. The compound of claim 1, wherein R² is H, CH₃, optionally substituted phenyl, optionally substituted (4- to 10-membered heterocyclyl)oxy, optionally substituted 4- to 10-membered heterocyclyl, optionally substituted 5- to 10-membered heteroaryl, C₃-C₈ cycloalkyl, or optionally substituted C₃-C₈ cycloalkenyl.

11. (canceled)

12. The compound of claim 10, wherein R² is

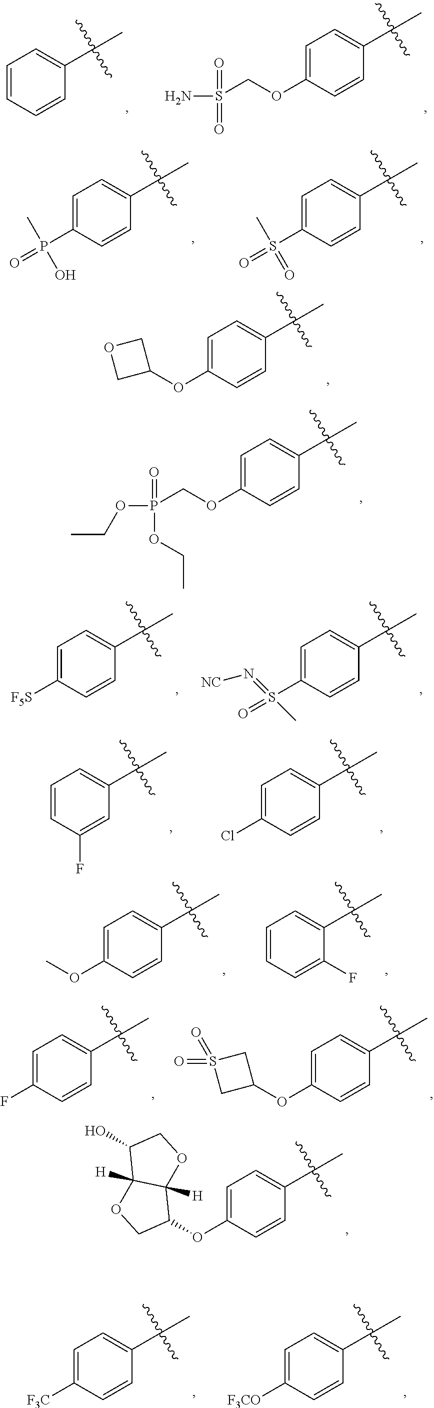

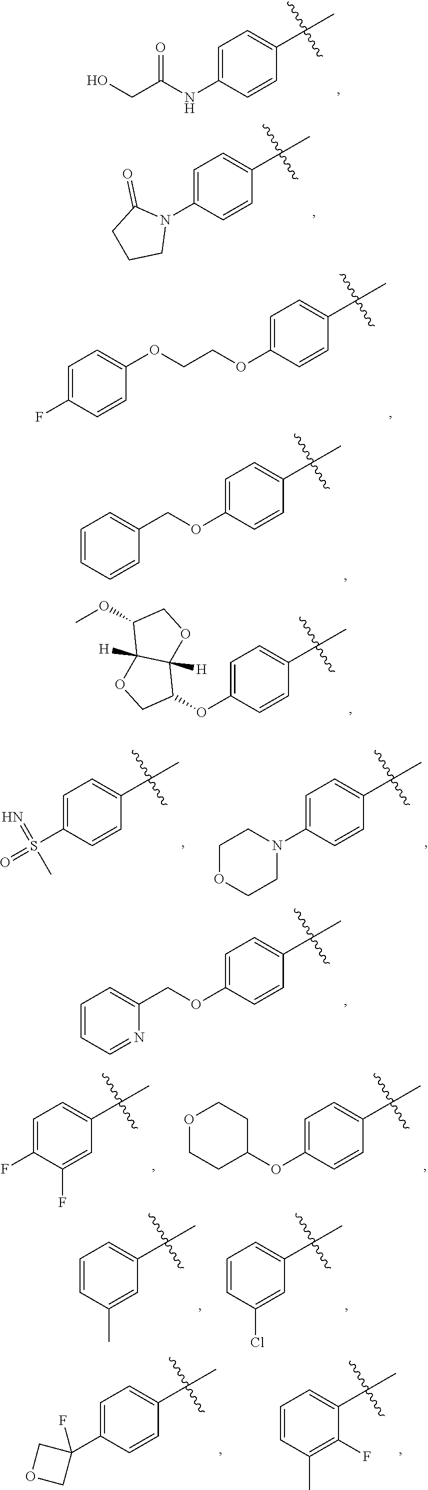

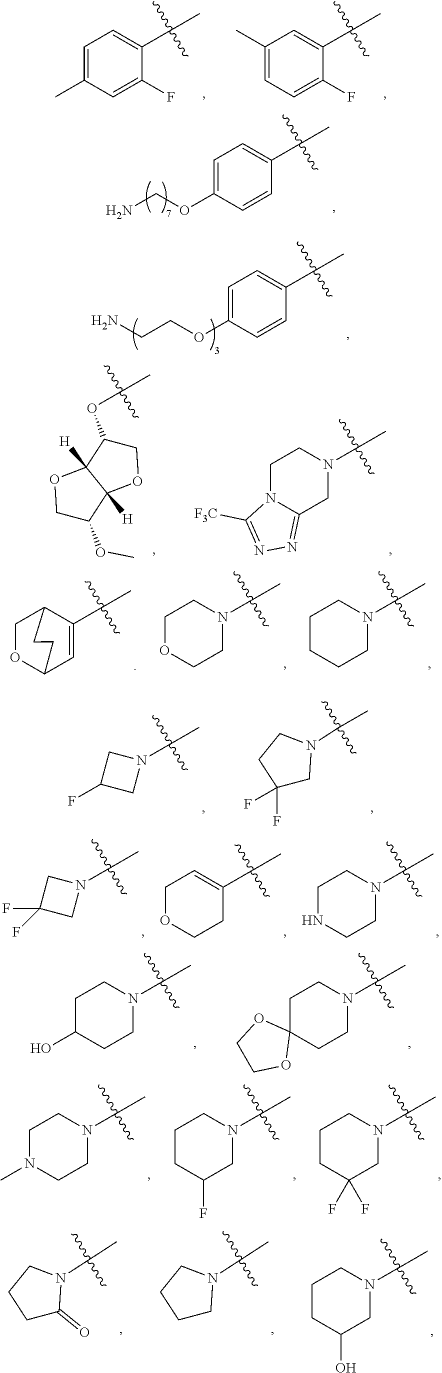

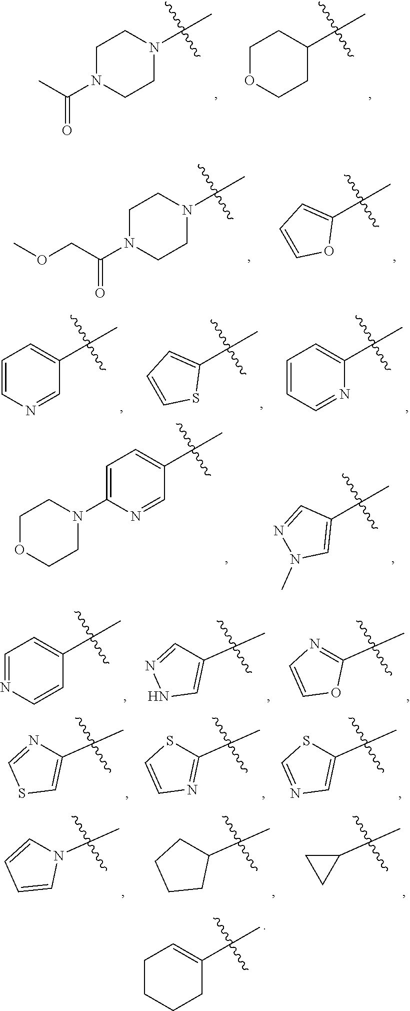

13-24. (canceled)

25. The compound of claim 1, wherein:
L¹ is a bond, NH, or NHC(O); and/or
L² is a bond, —CH₂—, —(CH₂)₂—, —(CH₂)₃—, —(CH₂)₄—, —(CH₂)₅—, 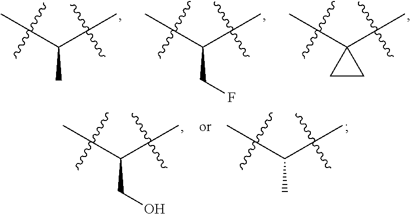
and/or
L³ is a bond, NHC(O), or C(O).

26-34. (canceled)

35. The compound of claim 1, where B is optionally substituted C₆-C₁₄ aryl or optionally 5- to 10-membered heteroaryl.

36. The compound of claim 35, wherein the compound is:
(A) a compound of Formula (II): 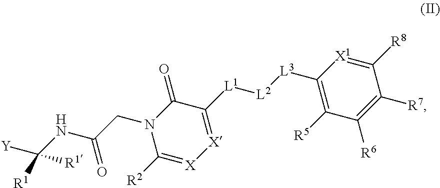
or a pharmaceutically acceptable salt thereof, wherein
X¹ is CR⁹ or N;
each of R⁵, R⁶, and R⁹ is independently selected from H, halo, CN, SF₅, optionally substituted C₁-C₆ alkyl, optionally substituted C₁-C₆ alkoxy, S(O)(NH)CH₃, S(O)₂CH₃, and S(O)(NCN)CH₃; and
each of R⁷ and R⁸ is independently H, halo, CN, SF₅, optionally substituted C₁-C₆ alkyl, optionally substituted C₁-C₆ alkoxy, optionally substituted amino, S(O)(NH)CH₃, S(O)₂CH₃, S(O)(NCN)CH₃, optionally substituted C₃-C₈ cycloalkyl, optionally substituted C₆-C₁₄ aryloxy, optionally substituted C₆-C₁₄ aryl, optionally substituted 5- to 10-membered heteroaryl, optionally substituted (5- to 10-membered heteroaryl)oxy, or optionally substituted (4- to 10-membered heterocyclyl)oxy, provided that no more than one of R⁷ and R⁸ is optionally substituted C₆-C₁₄ aryloxy, optionally substituted C₆-C₁₄ aryl, optionally substituted 5- to 10-membered heteroaryl, optionally substituted (5- to 10-membered heteroaryl)oxy, or optionally substituted (4- to 10-membered heterocyclyl)oxy; or
R⁷ and R⁸, together with the atoms to which each is attached, form optionally substituted 5- to 6-membered heterocyclyl, optionally substituted 5- to 10-membered heteroaryl, or optionally substituted C₆-C₁₄ aryl; or
R⁵ and A combine to form R⁷ is optionally substituted phenyl optionally substituted C₁-C₂ alkylene;
R⁶ and R⁹ combine to form (C₂-C₆alkylene)(C₆-C₁₄arylene)(C₂-C₆alkylene), and each of R₅, R₇, and R₈ is H; or
(B) a compound of Formula (III): 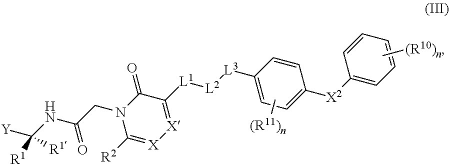
or a Pharmaceutically acceptable salt thereof, wherein
X² is O; C(R^{h})₂, wherein each R^{h} is independently hydrogen, halo, or optionally substituted C₁-C₆ alkyl, or both R^{h} combine to form oxo; S(O)₂, or NR^{h};
m is selected from 0, 1, 2, 3, 4, and 5;
n is selected from 0, 1, 2, 3, and 4; and
each R¹⁰ and R¹¹ is independently halo, CN, optionally substituted C₁-C₆ alkyl, optionally substituted C₁-C₆ alkoxy, or optionally substituted C₃-C₈ cycloalkyl; or
(C) a compound of Formula (IV) 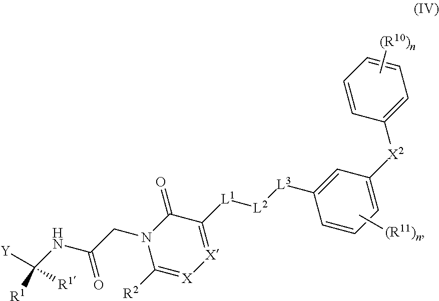
or a pharmaceutically acceptable salt thereof, wherein
X² is O; C(R^{h})₂, wherein each R^{h} is independently hydrogen, halo, or optionally substituted C₁-C₆ alkyl, or both R^{h} combine to form oxo; S(O)₂, or NR^{h};
m is selected from 0, 1, 2, 3, 4, and 5;
n is selected from 0, 1, 2, 3, and 4; and
each R¹⁰ and R¹¹ is independently halo, CN, optionally substituted C₁-C₆ alkyl, optionally substituted C₁-C₆ alkoxy, or optionally substituted C₃-C₈ cycloalkyl.

37. (canceled)

38. The compound of claim 36, wherein the compound is a compound of Formula (II) or a pharmaceutically acceptable salt thereof, wherein:
X¹ is CH; and/or
R⁵ is H; and/or
R⁶ is H, optionally substituted C₁-C₆ alkoxy, or halo; and/or
R⁸ is H and R⁷ is H, optionally substituted phenyl, optionally substituted 5- to 10-membered heteroaryl, optionally substituted (5- to 10-membered heteroaryl)oxy, optionally substituted (4- to 10-membered heterocyclyl)oxy, optionally substituted C₃-C₈ cycloalkyl, or optionally substituted 4- to 10-membered heterocyclyl; or R⁷ is H and R⁸ is optionally substituted phenyl, or optionally substituted 5- to 10-membered heteroaryl.

39-40. (canceled)

41. The compound of claim 38, wherein:
R⁶ is H, OCH₃, or F; and/or
R⁸ is H and R⁷ is 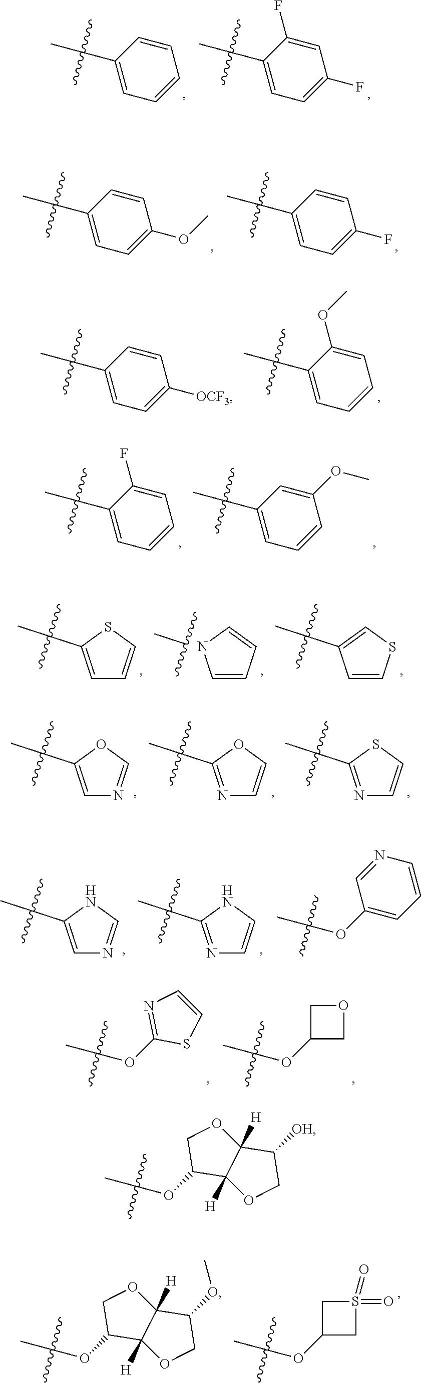 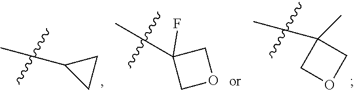
R⁷ is H and R⁸ is

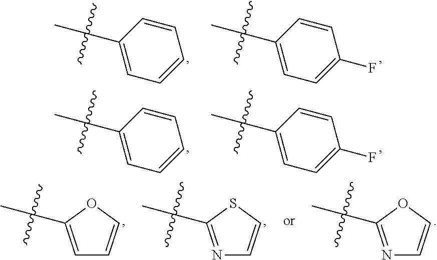

42-67. (canceled)

68. The compound of claim 36, wherein the compound is a compound of Formula (III) or Formula (IV), or a pharmaceutically acceptable salt thereof, wherein X² is O or C(O).

69-71. (canceled)

72. The compound of claim 1, wherein B is optionally substituted C₃-C₁₄ carbocyclyl or optionally substituted 5- to 14-membered heterocyclyl.

73. The compound of claim 72, wherein the compound is a compound of Formula (V) or Formula (VI): 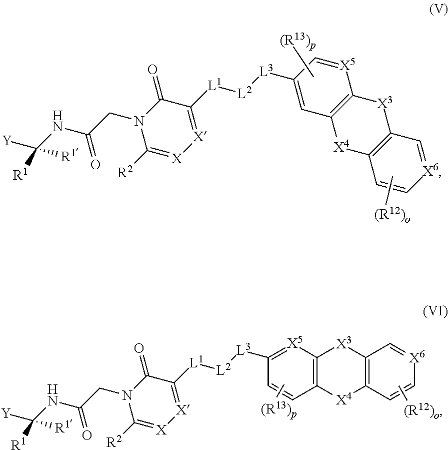
or a pharmaceutically acceptable salt thereof, wherein
each of X³ and X⁴ is independently a bond; 0; S; C(R^{d})₂, wherein each R^{d} is independently H, OH, halo, optionally substituted C₁-C₆ alkyl, or optionally substituted C₁-C₆ alkoxy, or both R^{d} combine to form oxo; NR^{e}, wherein R^{e} is H or C₁-C₆ alkyl; or SO₂;
X⁵ is CH, CR¹³, or N;
X⁶ is CH, CR¹², or N;
o is selected from 0, 1, 2, and 3;
p is selected from 0, 1, and 2; and
each R¹² and R¹³ is independently halo, optionally substituted C₁-C₆ alkyl, optionally substituted C₁-C₆ alkoxy, or optionally substituted C₃-C₈ cycloalkyl.

74. (canceled)

75. The compound of claim 73, wherein X³ is O, S, or CF₂; and/or X⁴ is a bond.

76-81. (canceled)

82. The compound of claim 1, wherein B is

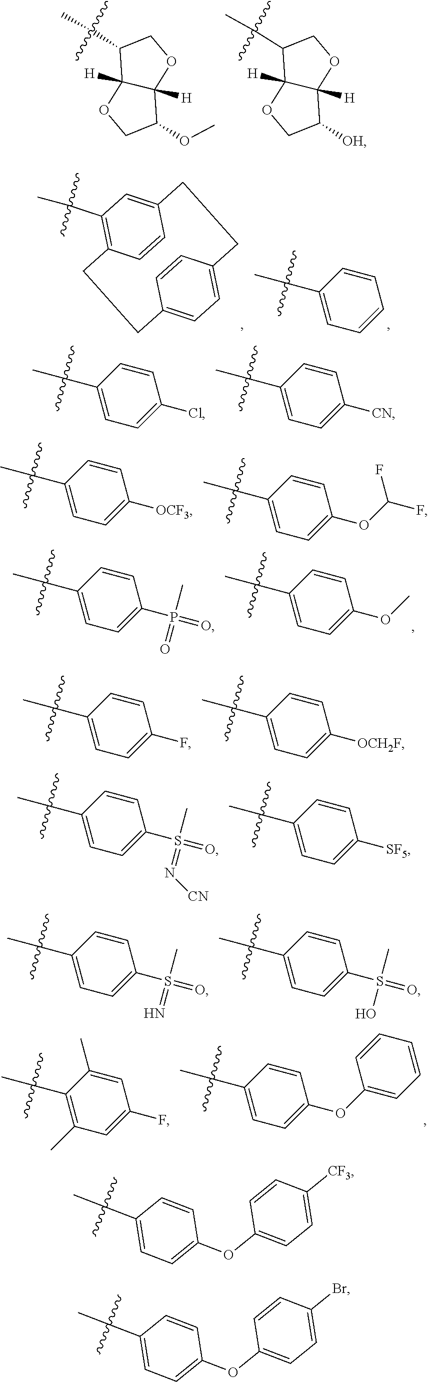

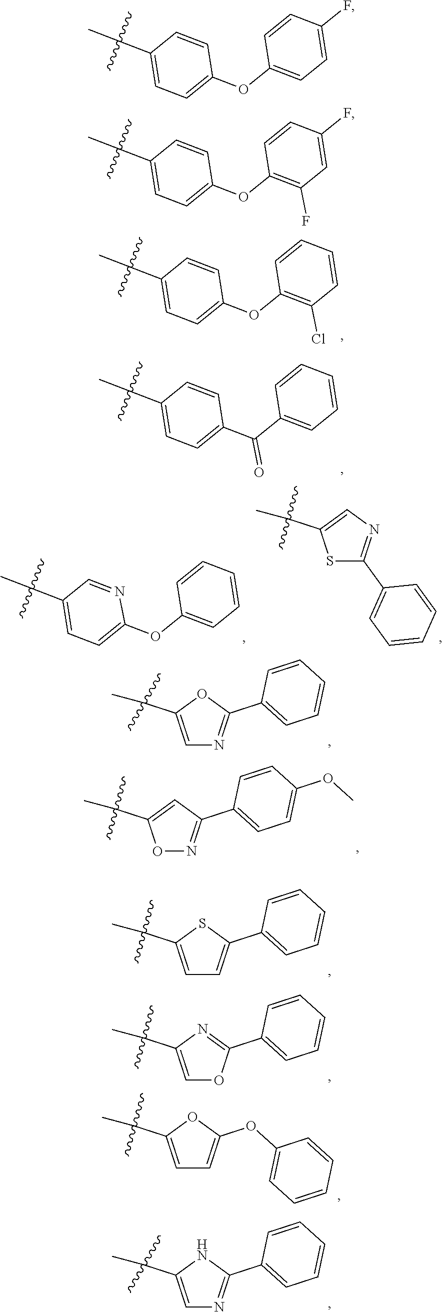

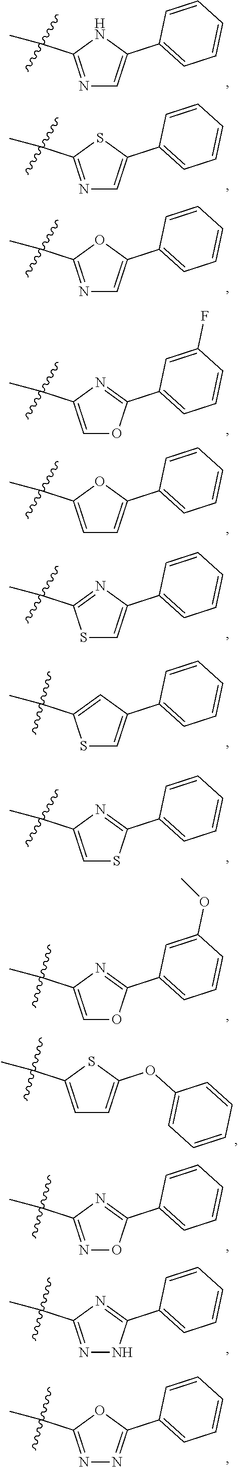

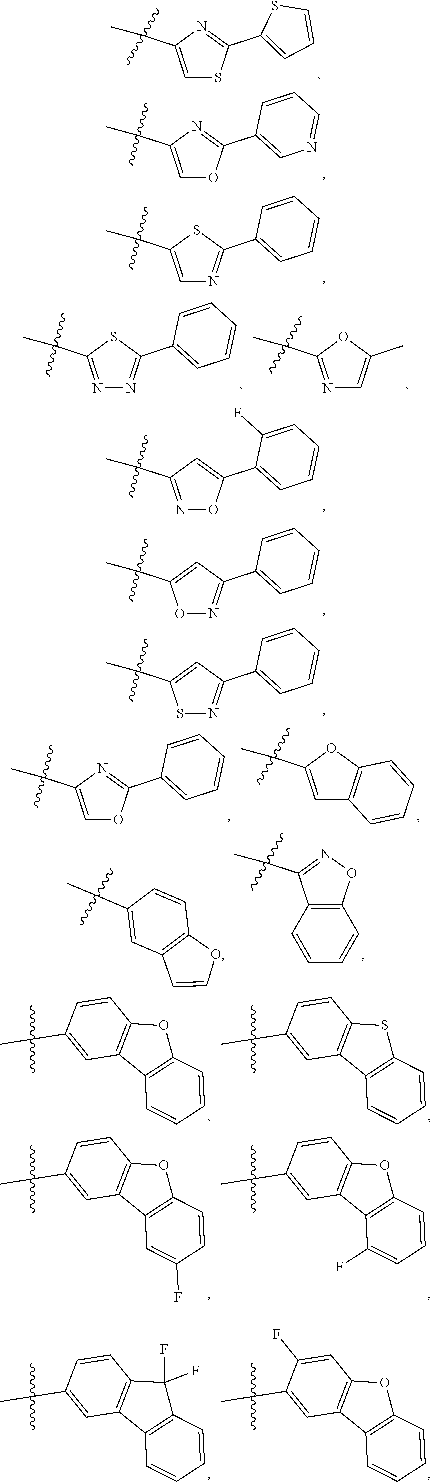

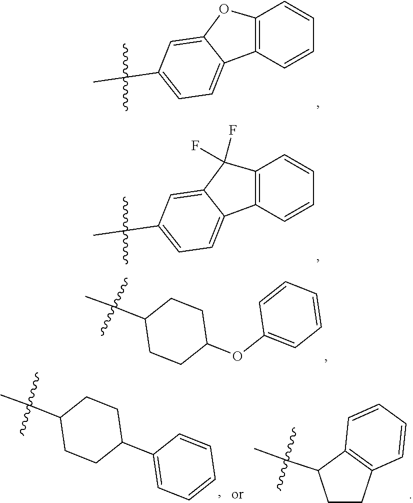

83. The compound of claim 1, wherein Y is

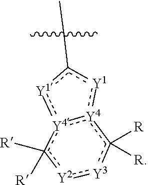

84. The compound of claim 83, wherein:
(A) Y is 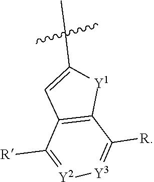
wherein:
Y² is N or CH; and/or
Y³ is N or CH; and/or
Y¹ is NH or S; and/or
R is H; and/or
R′ is H; or
(B) Y is 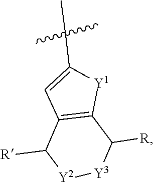
wherein:
Y¹ is S; and/or
Y² is NH or CH₂; and/or
Y³ is NH or CH₂; and/or
R is H; and/or
R′ is H.

85-92. (canceled)

93. The compound claim 1, wherein Y is

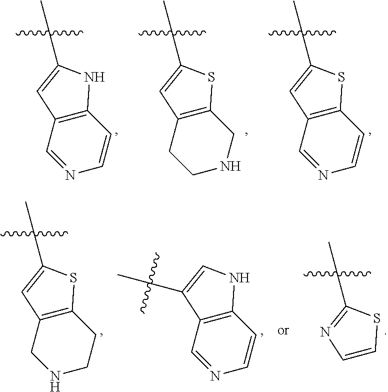 [SMILES: *c1cc2cnccc2[nH]1 ; *c1cc2c(s1)CNCC2 ; *c1cc2cnccc2s1 ; *c1cc2c(s1)CCNC2 ; *c1c[nH]c2ccncc12 ; *c1nccs1]

94. A compound selected from: 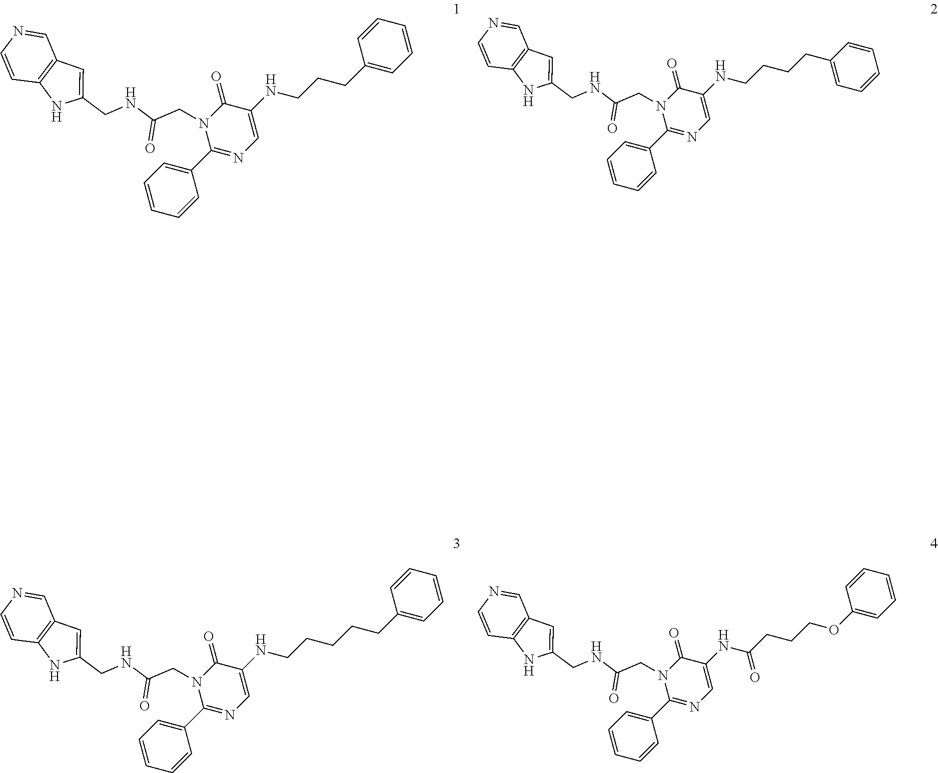 [SMILES: O=C(Cn1c(-c2ccccc2)ncc(NCCCc2ccccc2)c1=O)NCc1cc2cnccc2[nH]1 ; O=C(Cn1c(-c2ccccc2)ncc(NCCCCc2ccccc2)c1=O)NCc1cc2cnccc2[nH]1 ; O=C(Cn1c(-c2ccccc2)ncc(NCCCCCc2ccccc2)c1=O)NCc1cc2cnccc2[nH]1 ; O=C(Cn1c(-c2ccccc2)ncc(NC(=O)CCCOc2ccccc2)c1=O)NCc1cc2cnccc2[nH]1] 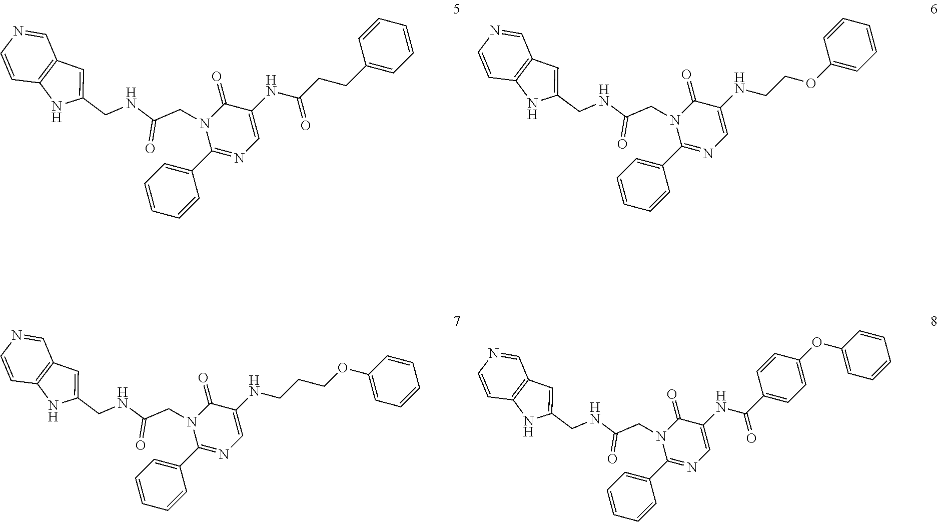 [SMILES: O=C(Cn1c(-c2ccccc2)ncc(NC(=O)CCc2ccccc2)c1=O)NCc1cc2cnccc2[nH]1 ; O=C(Cn1c(-c2ccccc2)ncc(NCCOc2ccccc2)c1=O)NCc1cc2cnccc2[nH]1 ; O=C(Cn1c(-c2ccccc2)ncc(NCCCOc2ccccc2)c1=O)NCc1cc2cnccc2[nH]1 ; O=C(Cn1c(-c2ccccc2)ncc(NC(=O)c2ccc(Oc3ccccc3)cc2)c1=O)NCc1cc2cnccc2[nH]1] 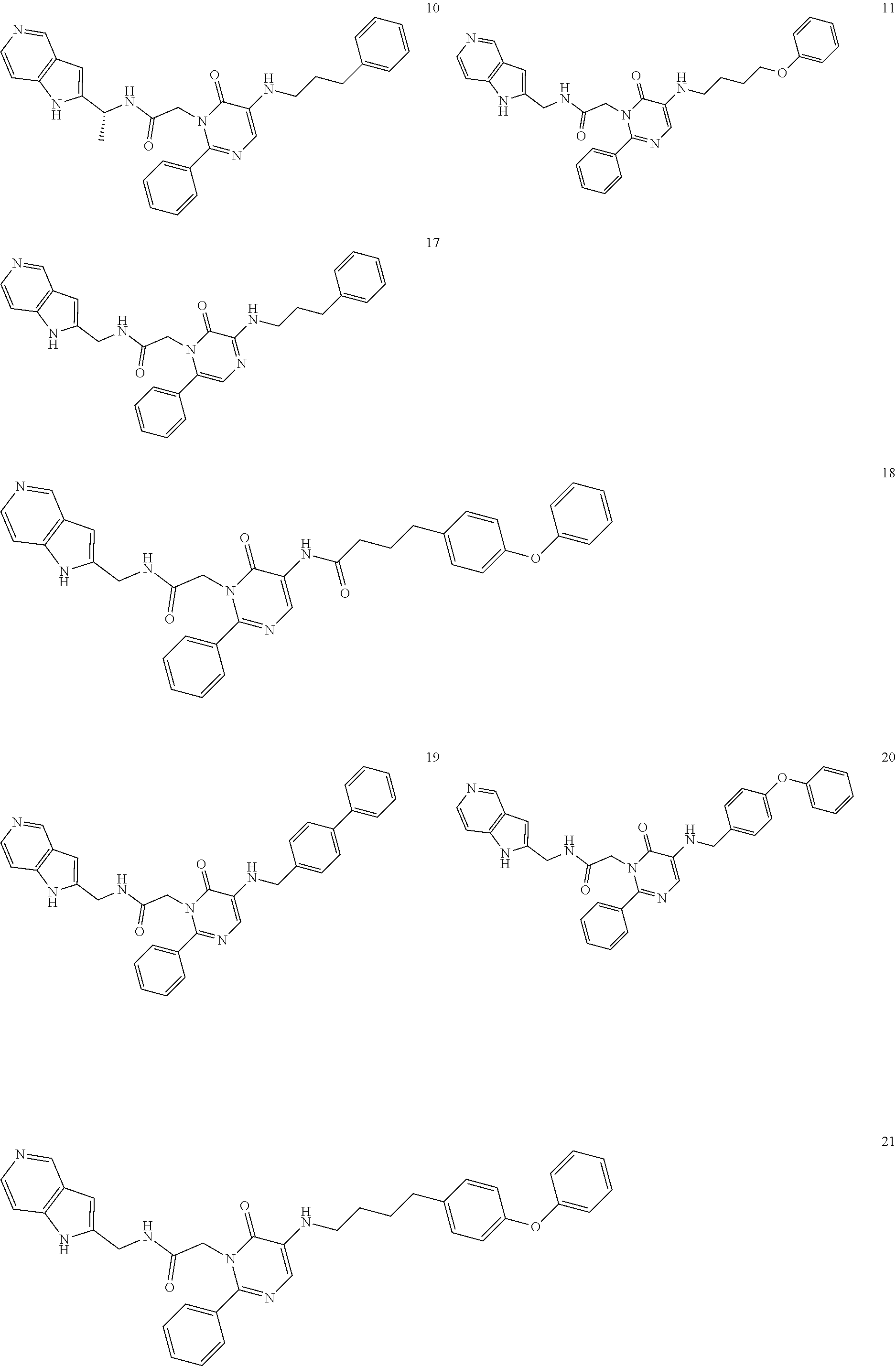 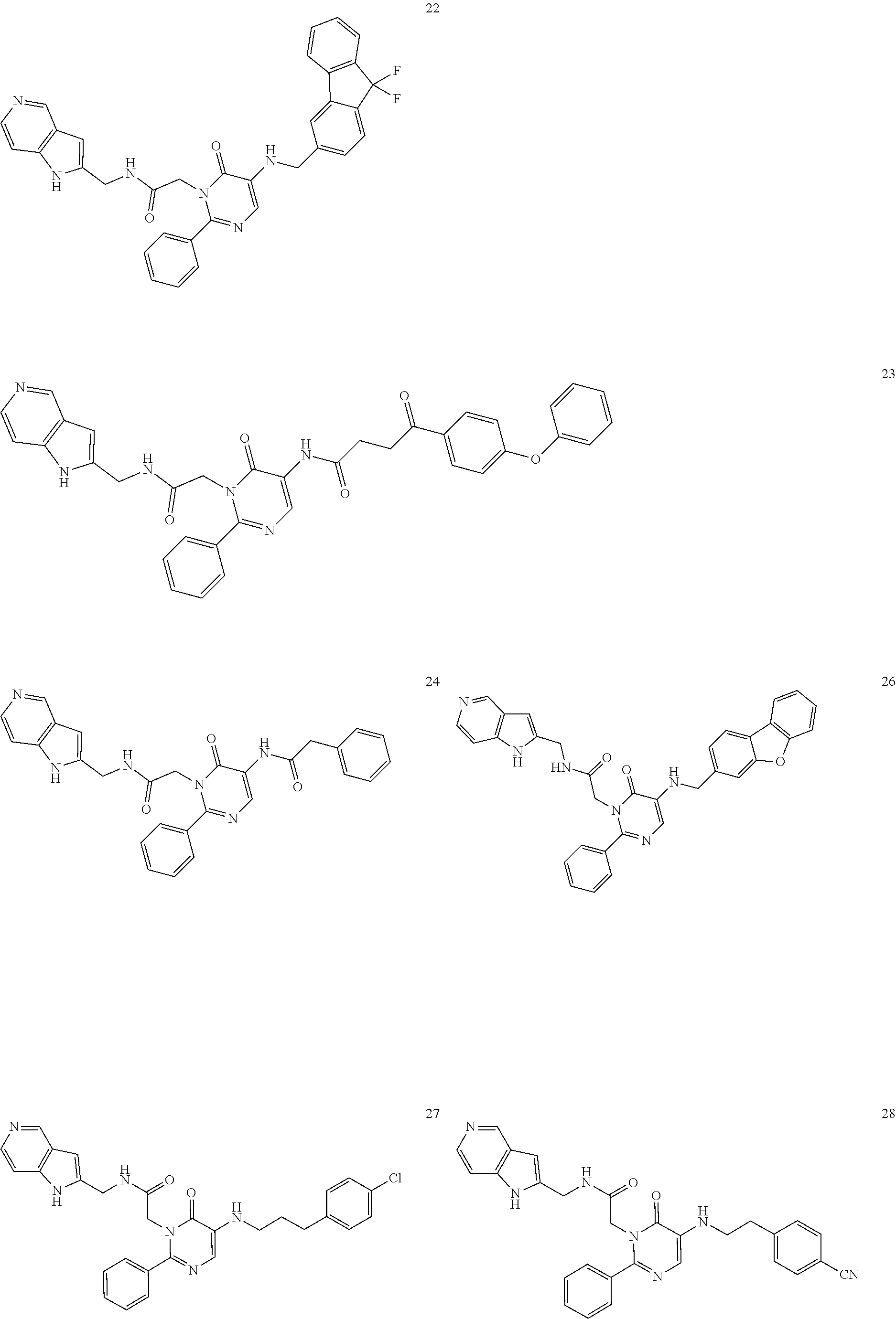 [SMILES: O=C(Cn1c(-c2ccccc2)ncc(NCc2ccc3c(c2)-c2ccccc2C3(F)F)c1=O)NCc1cc2cnccc2[nH]1 ; O=C(Cn1c(-c2ccccc2)ncc(NC(=O)CCC(=O)c2ccc(Oc3ccccc3)cc2)c1=O)NCc1cc2cnccc2[nH]1 ; O=C(Cn1c(-c2ccccc2)ncc(NC(=O)Cc2ccccc2)c1=O)NCc1cc2cnccc2[nH]1 ; O=C(Cn1c(-c2ccccc2)ncc(NCc2ccc3c(c2)oc2ccccc23)c1=O)NCc1cc2cnccc2[nH]1 ; O=C(Cn1c(-c2ccccc2)ncc(NCCCc2ccc(Cl)cc2)c1=O)NCc1cc2cnccc2[nH]1 ; N#Cc1ccc(CCNc2cnc(-c3ccccc3)n(CC(=O)NCc3cc4cnccc4[nH]3)c2=O)cc1] 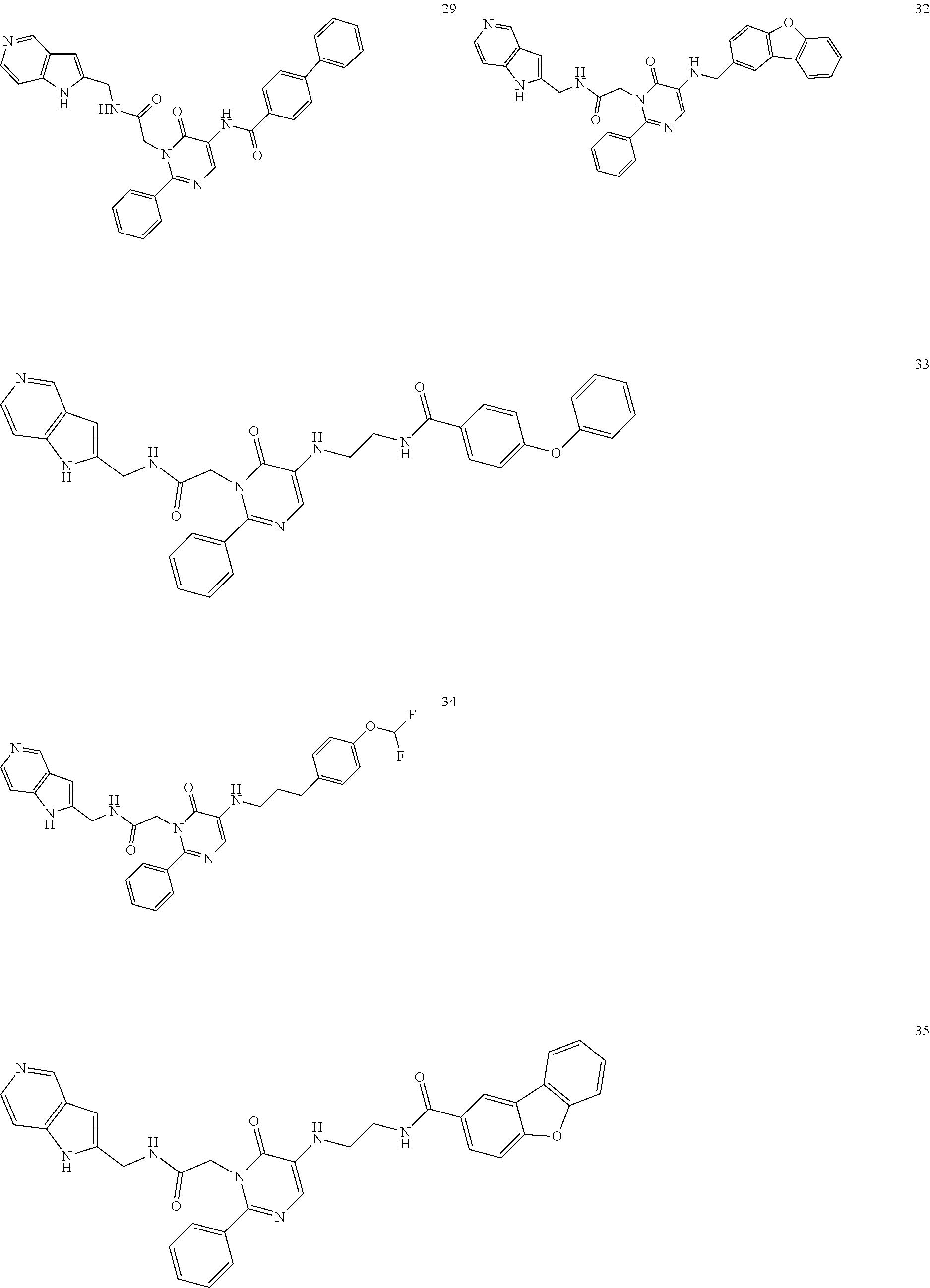 [SMILES: O=C(Cn1c(-c2ccccc2)ncc(NC(=O)c2ccc(-c3ccccc3)cc2)c1=O)NCc1cc2cnccc2[nH]1 ; O=C(Cn1c(-c2ccccc2)ncc(NCc2ccc3oc4ccccc4c3c2)c1=O)NCc1cc2cnccc2[nH]1 ; O=C(Cn1c(-c2ccccc2)ncc(NCCNC(=O)c2ccc(Oc3ccccc3)cc2)c1=O)NCc1cc2cnccc2[nH]1 ; O=C(Cn1c(-c2ccccc2)ncc(NCCCc2ccc(OC(F)F)cc2)c1=O)NCc1cc2cnccc2[nH]1 ; O=C(Cn1c(-c2ccccc2)ncc(NCCNC(=O)c2ccc3oc4ccccc4c3c2)c1=O)NCc1cc2cnccc2[nH]1] 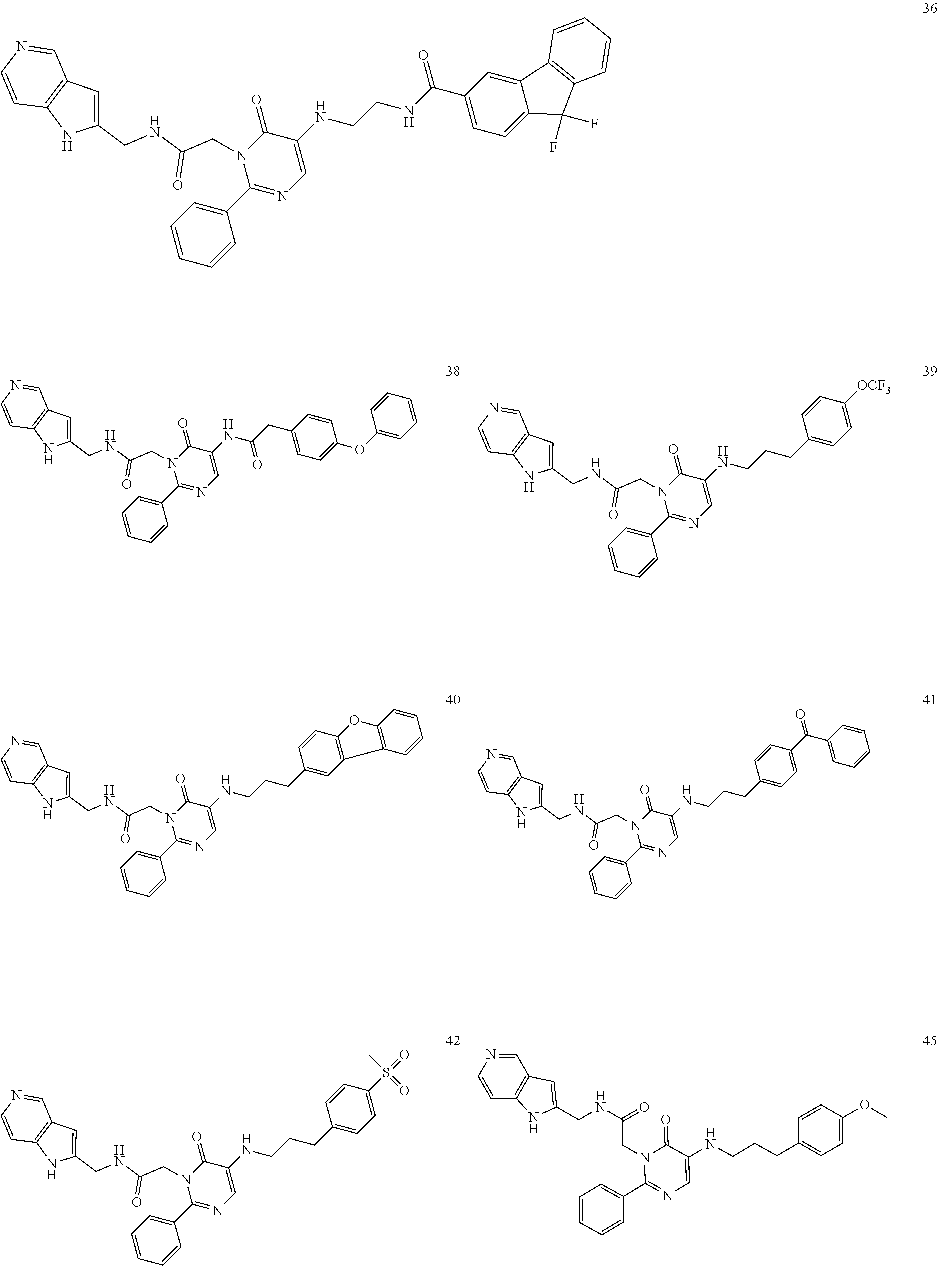 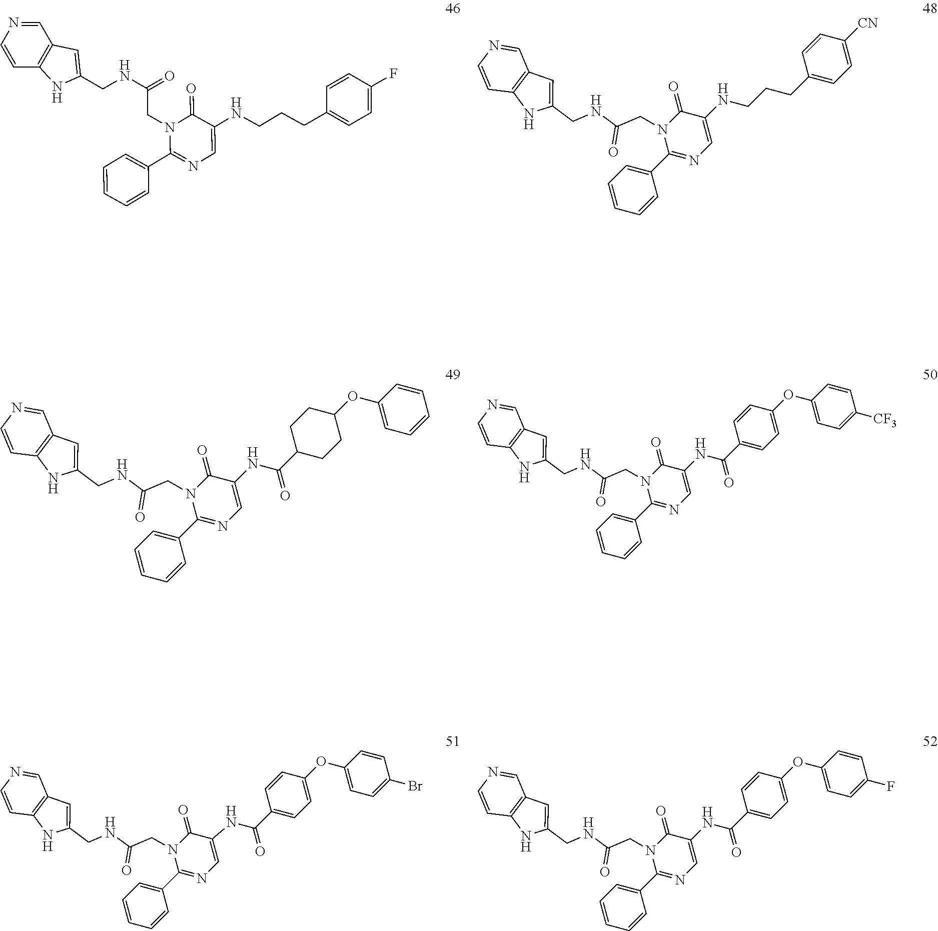 [SMILES: O=C(Cn1c(-c2ccccc2)ncc(NCCCc2ccc(F)cc2)c1=O)NCc1cc2cnccc2[nH]1 ; N#Cc1ccc(CCCNc2cnc(-c3ccccc3)n(CC(=O)NCc3cc4cnccc4[nH]3)c2=O)cc1 ; O=C(Cn1c(-c2ccccc2)ncc(NC(=O)C2CCC(Oc3ccccc3)CC2)c1=O)NCc1cc2cnccc2[nH]1 ; O=C(Cn1c(-c2ccccc2)ncc(NC(=O)c2ccc(Oc3ccc(C(F)(F)F)cc3)cc2)c1=O)NCc1cc2cnccc2[nH]1 ; O=C(Cn1c(-c2ccccc2)ncc(NC(=O)c2ccc(Oc3ccc(Br)cc3)cc2)c1=O)NCc1cc2cnccc2[nH]1 ; O=C(Cn1c(-c2ccccc2)ncc(NC(=O)c2ccc(Oc3ccc(F)cc3)cc2)c1=O)NCc1cc2cnccc2[nH]1] 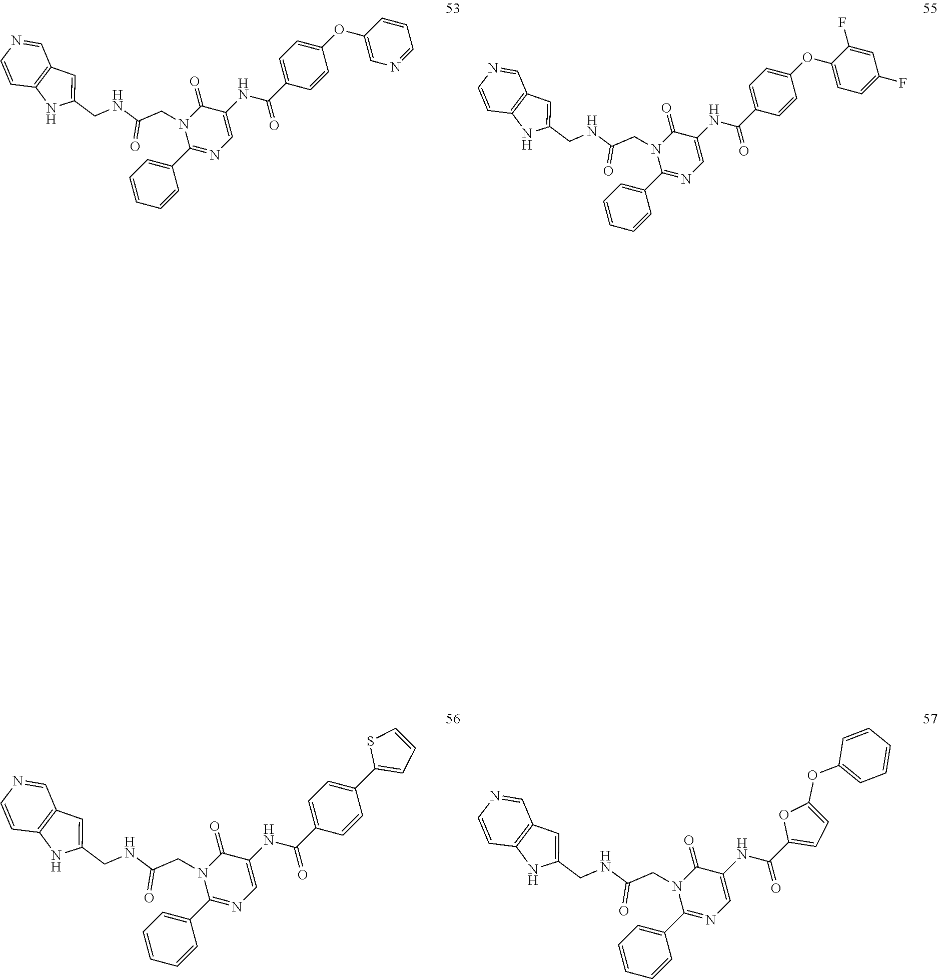 [SMILES: O=C(Cn1c(-c2ccccc2)ncc(NC(=O)c2ccc(Oc3cccnc3)cc2)c1=O)NCc1cc2cnccc2[nH]1 ; O=C(Cn1c(-c2ccccc2)ncc(NC(=O)c2ccc(Oc3ccc(F)cc3F)cc2)c1=O)NCc1cc2cnccc2[nH]1 ; O=C(Cn1c(-c2ccccc2)ncc(NC(=O)c2ccc(-c3cccs3)cc2)c1=O)NCc1cc2cnccc2[nH]1 ; O=C(Cn1c(-c2ccccc2)ncc(NC(=O)c2ccc(Oc3ccccc3)o2)c1=O)NCc1cc2cnccc2[nH]1] 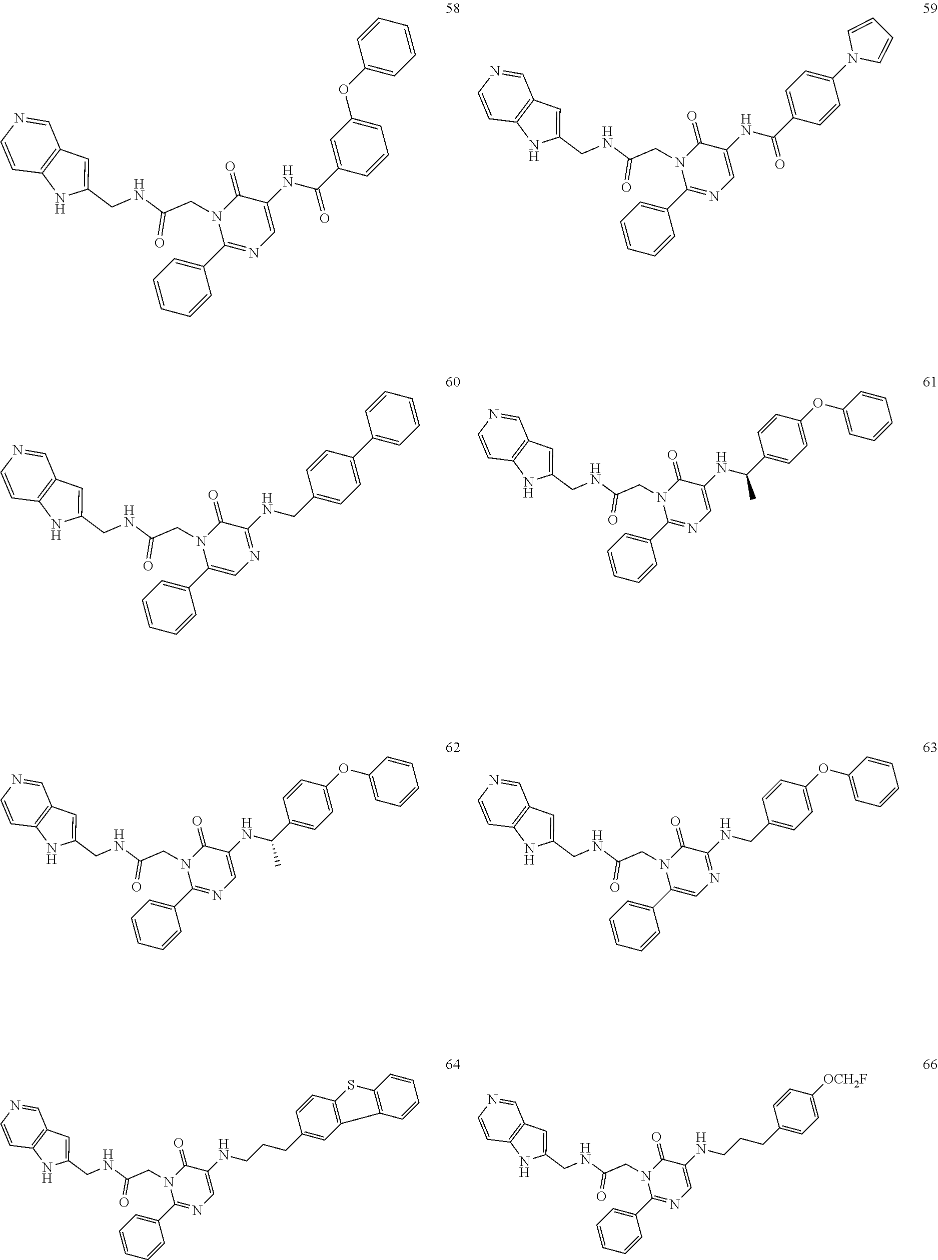 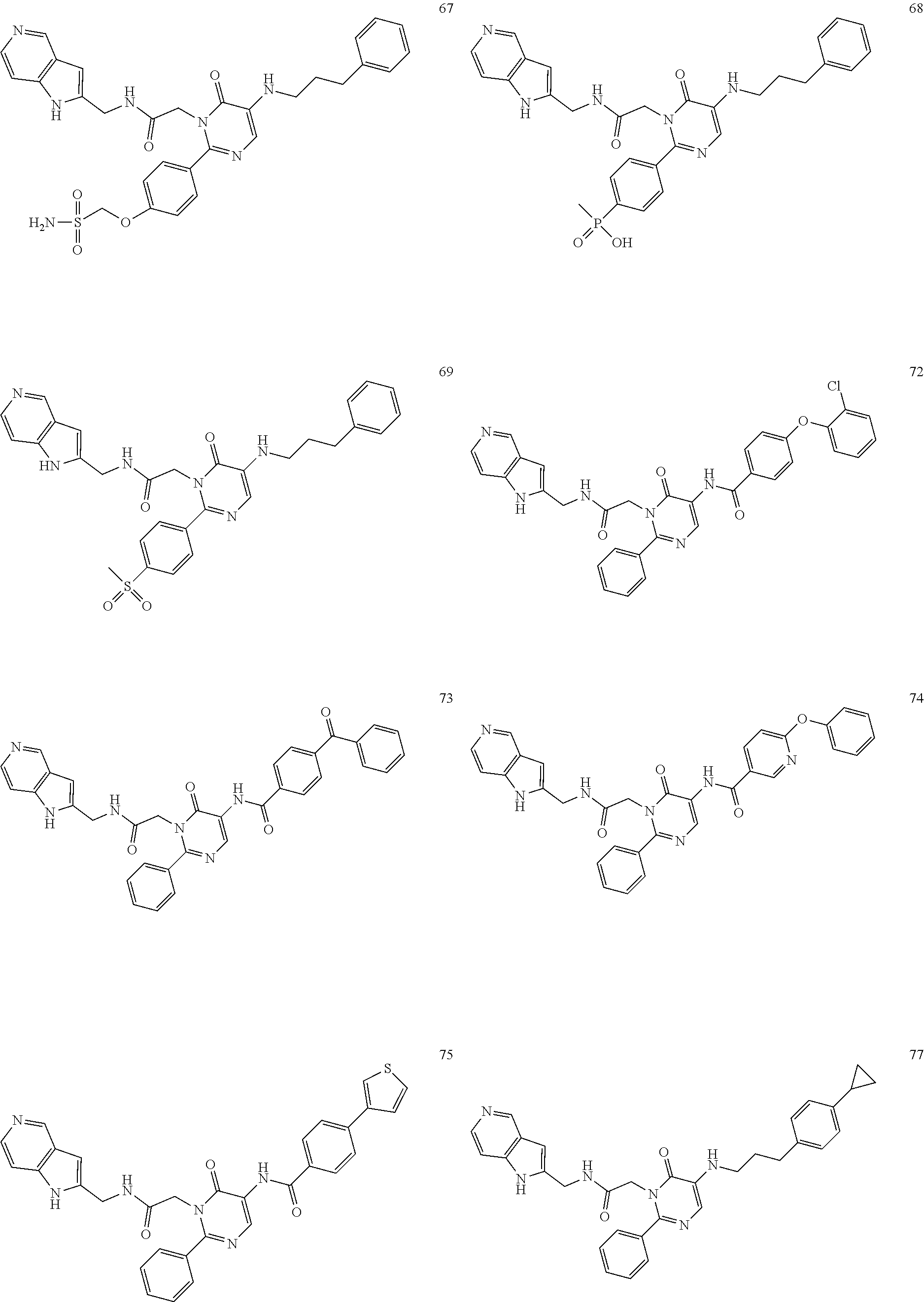 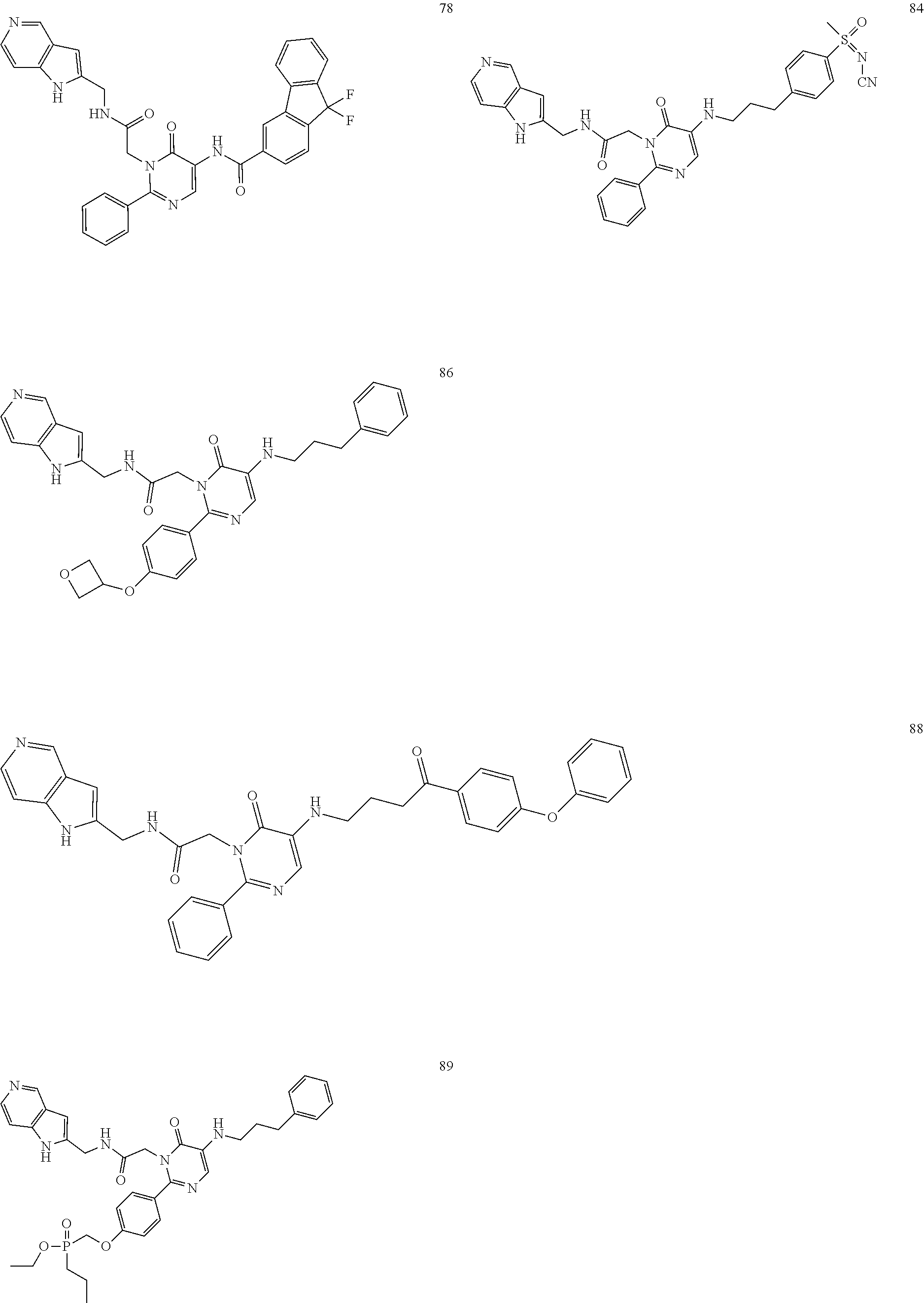 [SMILES: O=C(Cn1c(-c2ccccc2)ncc(NC(=O)c2ccc3c(c2)-c2ccccc2C3(F)F)c1=O)NCc1cc2cnccc2[nH]1 ; CS(=O)(=NC#N)c1ccc(CCCNc2cnc(-c3ccccc3)n(CC(=O)NCc3cc4cnccc4[nH]3)c2=O)cc1 ; O=C(Cn1c(-c2ccc(OC3COC3)cc2)ncc(NCCCc2ccccc2)c1=O)NCc1cc2cnccc2[nH]1 ; O=C(Cn1c(-c2ccccc2)ncc(NCCCC(=O)c2ccc(Oc3ccccc3)cc2)c1=O)NCc1cc2cnccc2[nH]1 ; CCCP(=O)(COc1ccc(-c2ncc(NCCCc3ccccc3)c(=O)n2CC(=O)NCc2cc3cnccc3[nH]2)cc1)OCC] 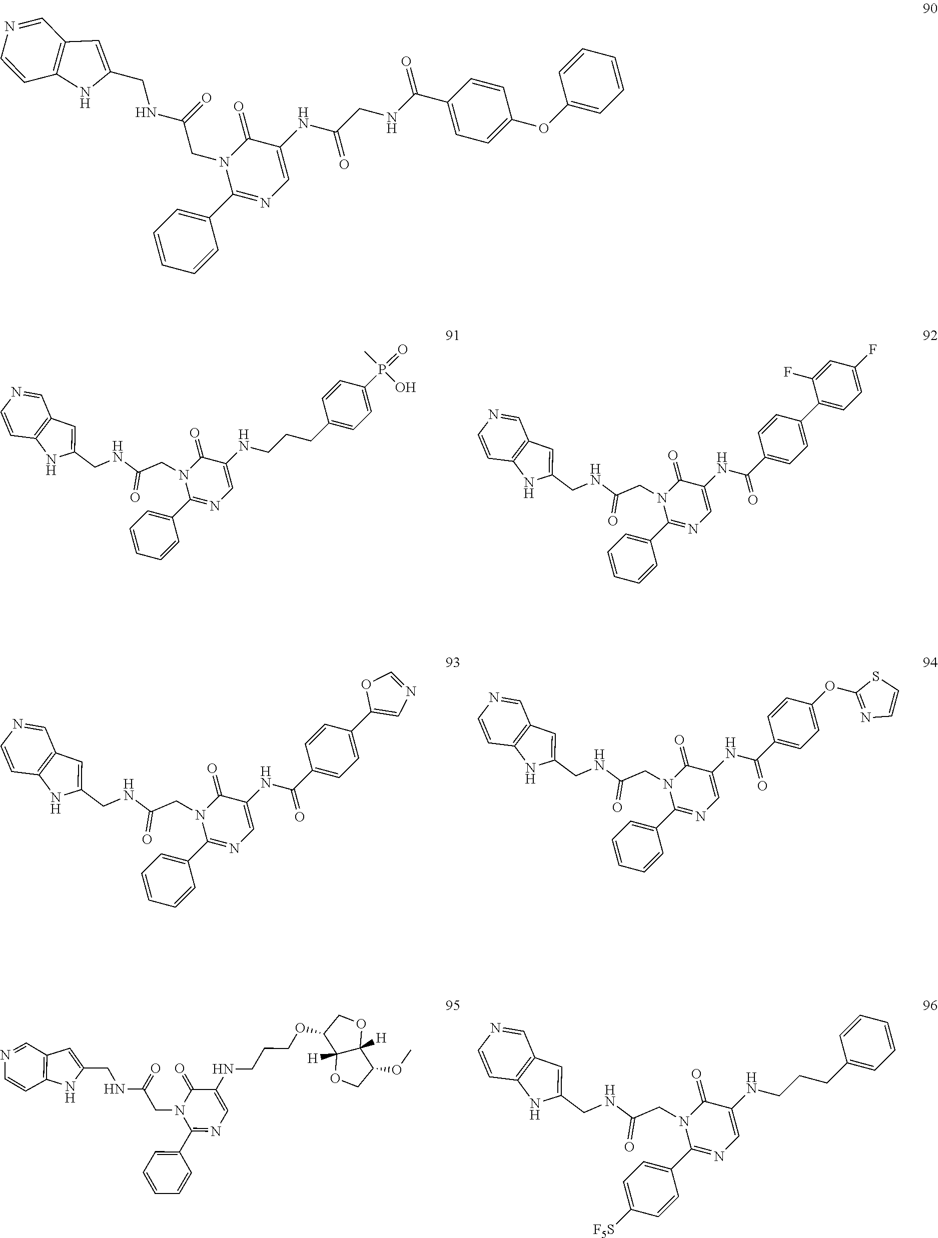 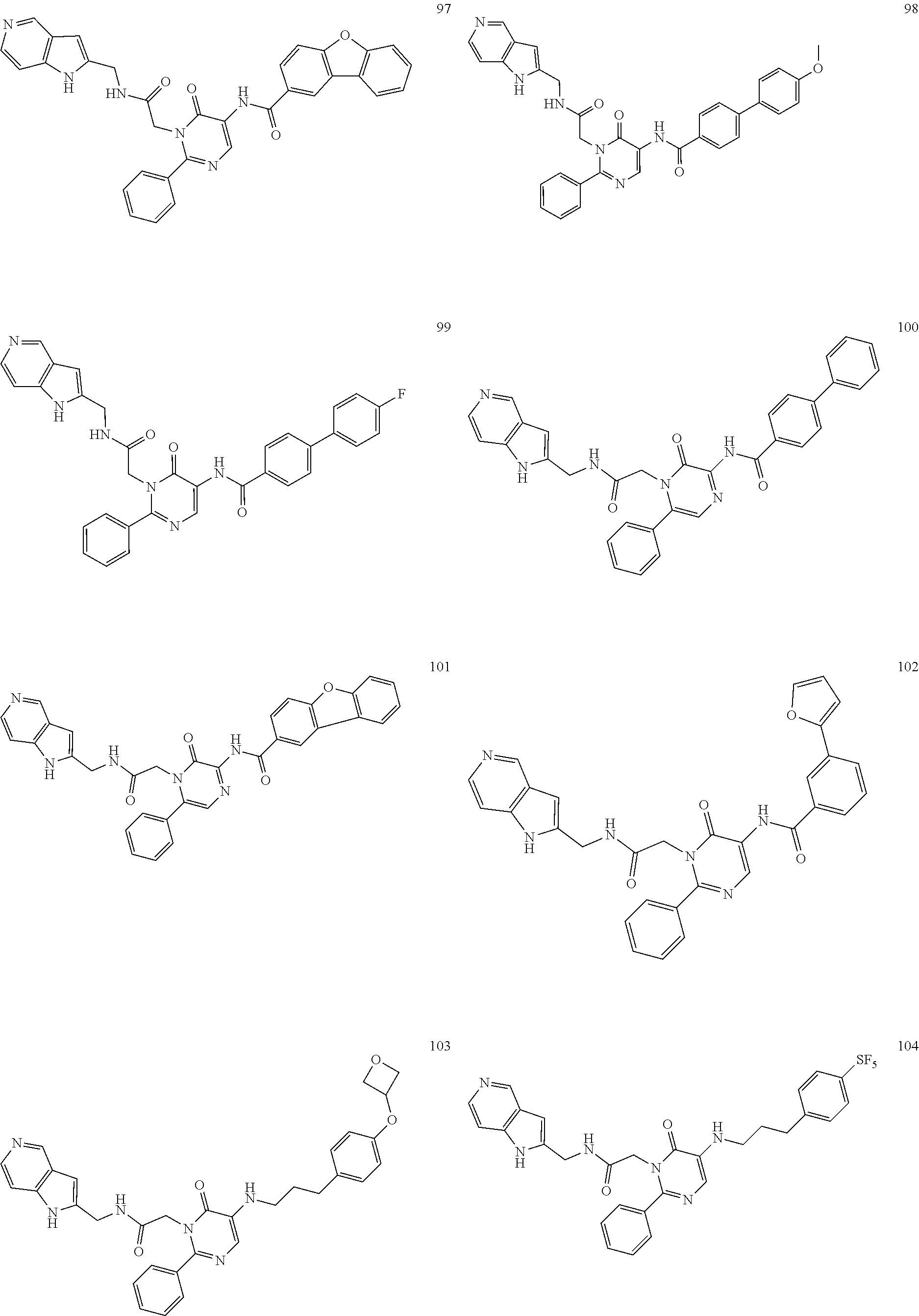 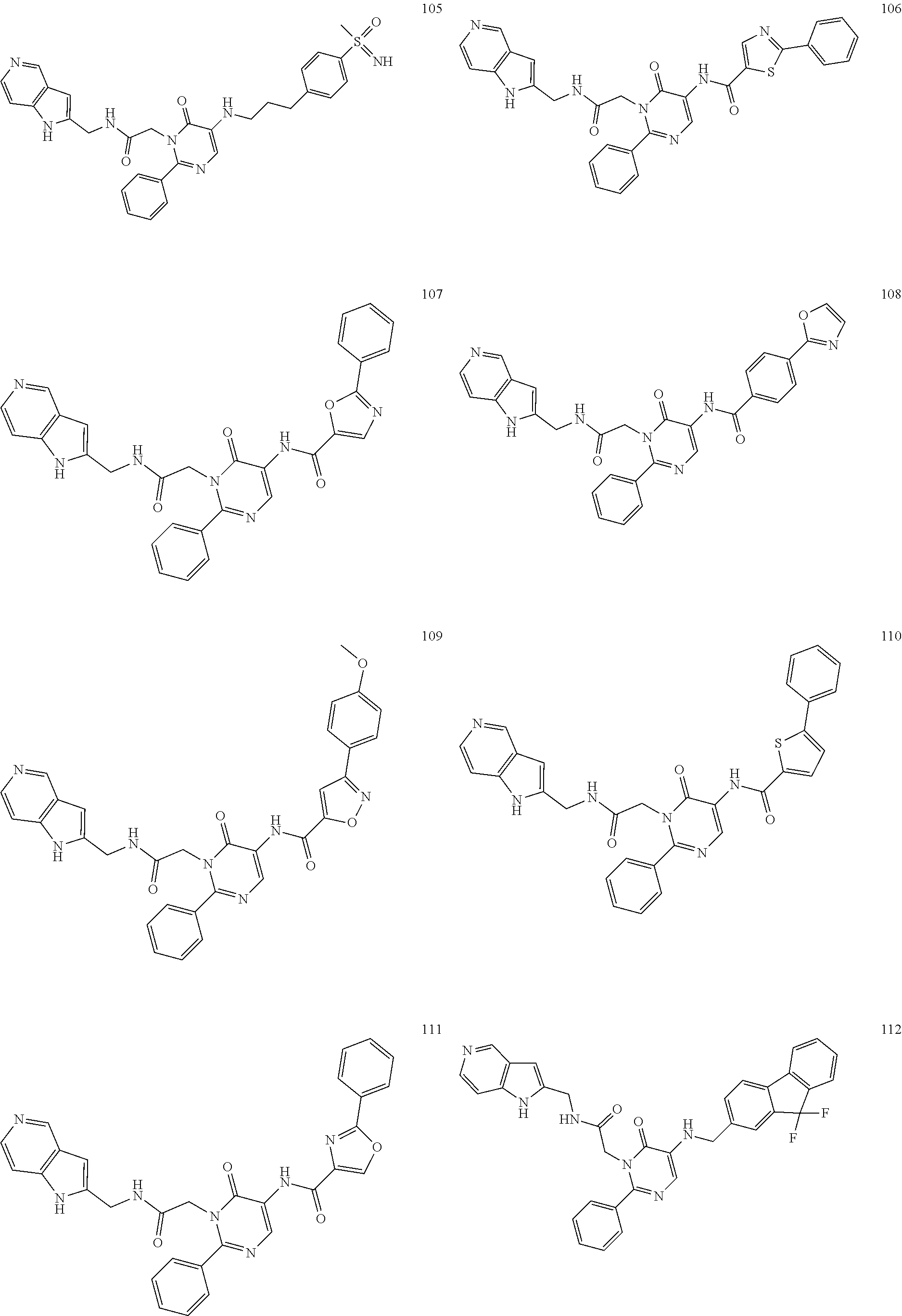 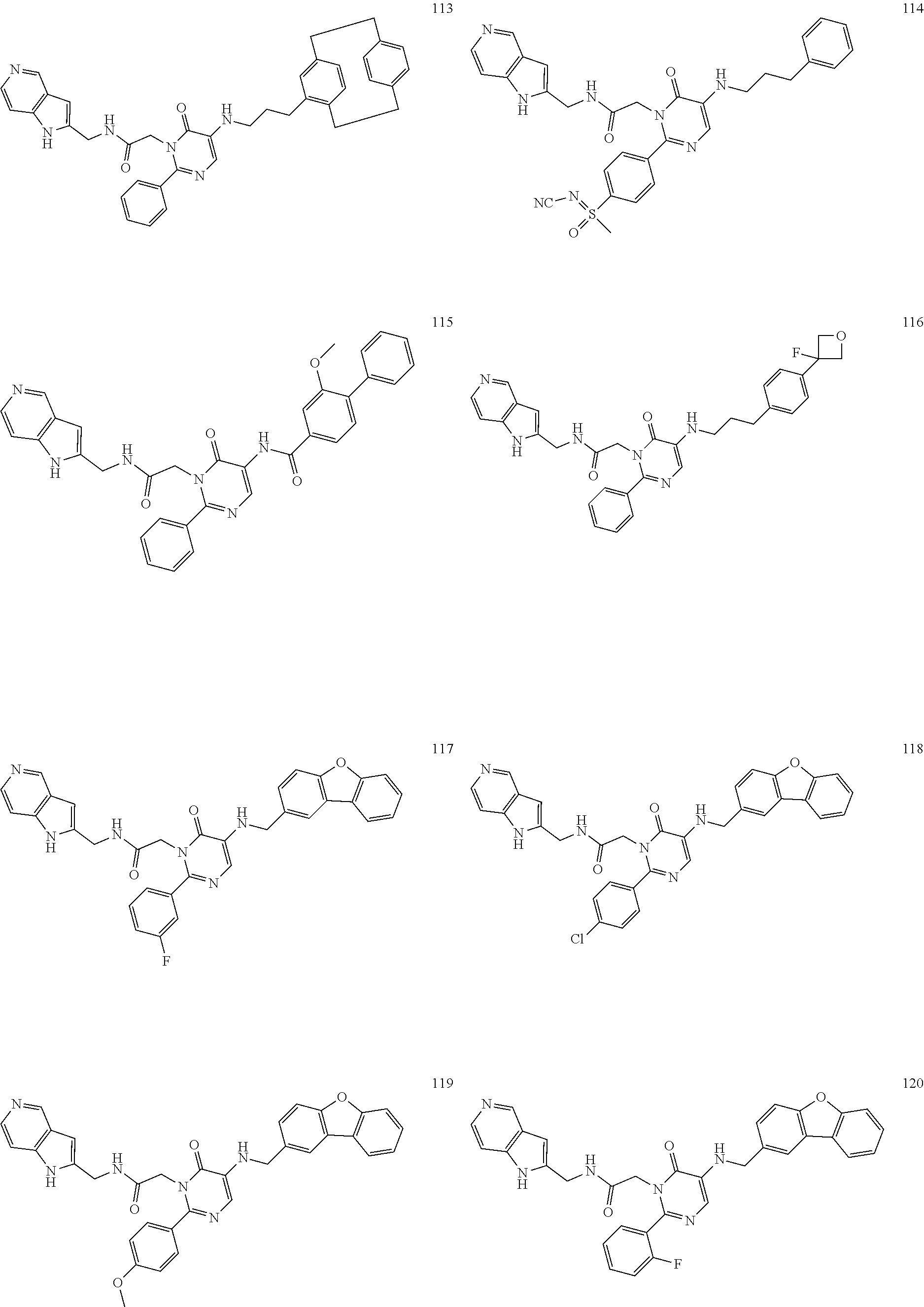 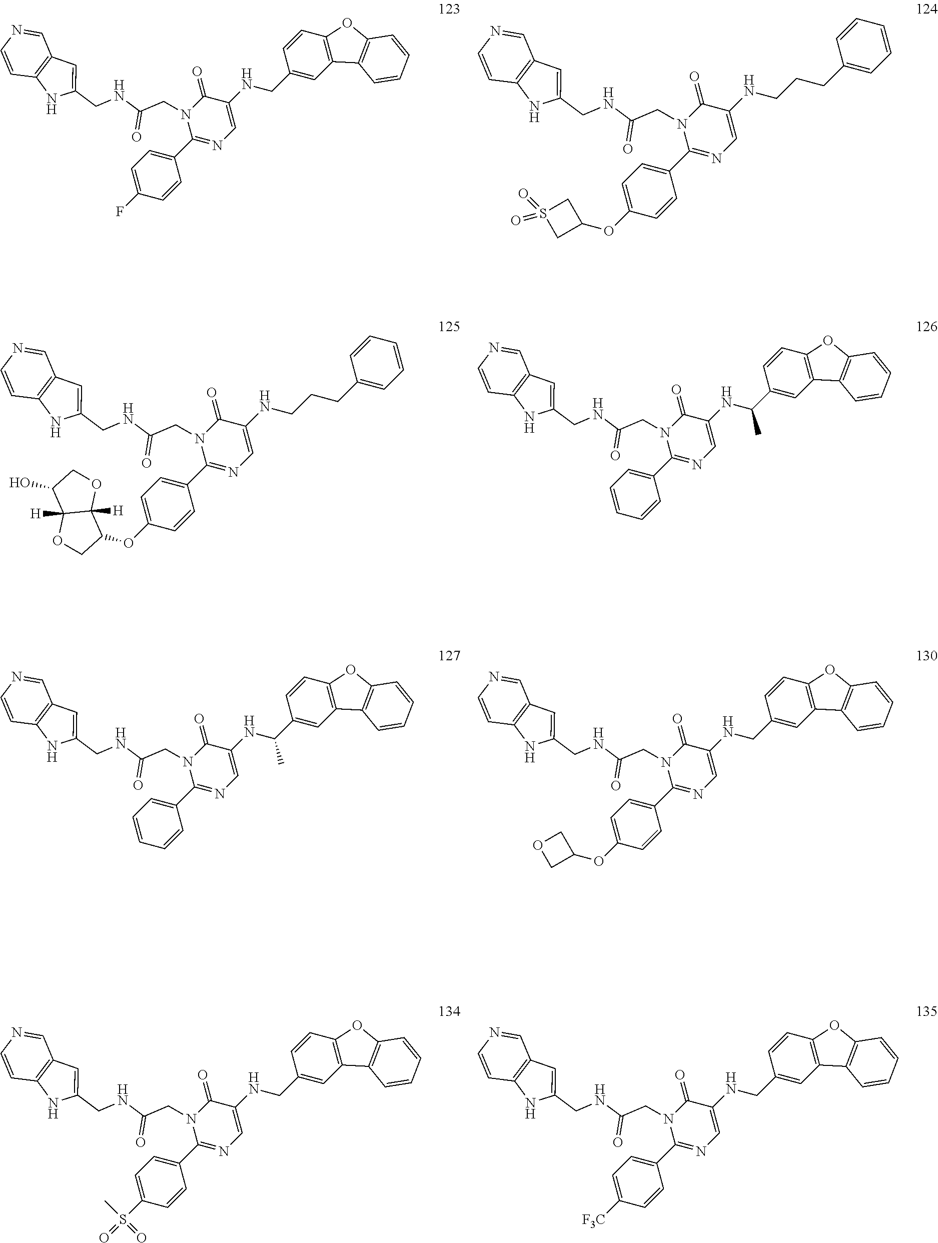 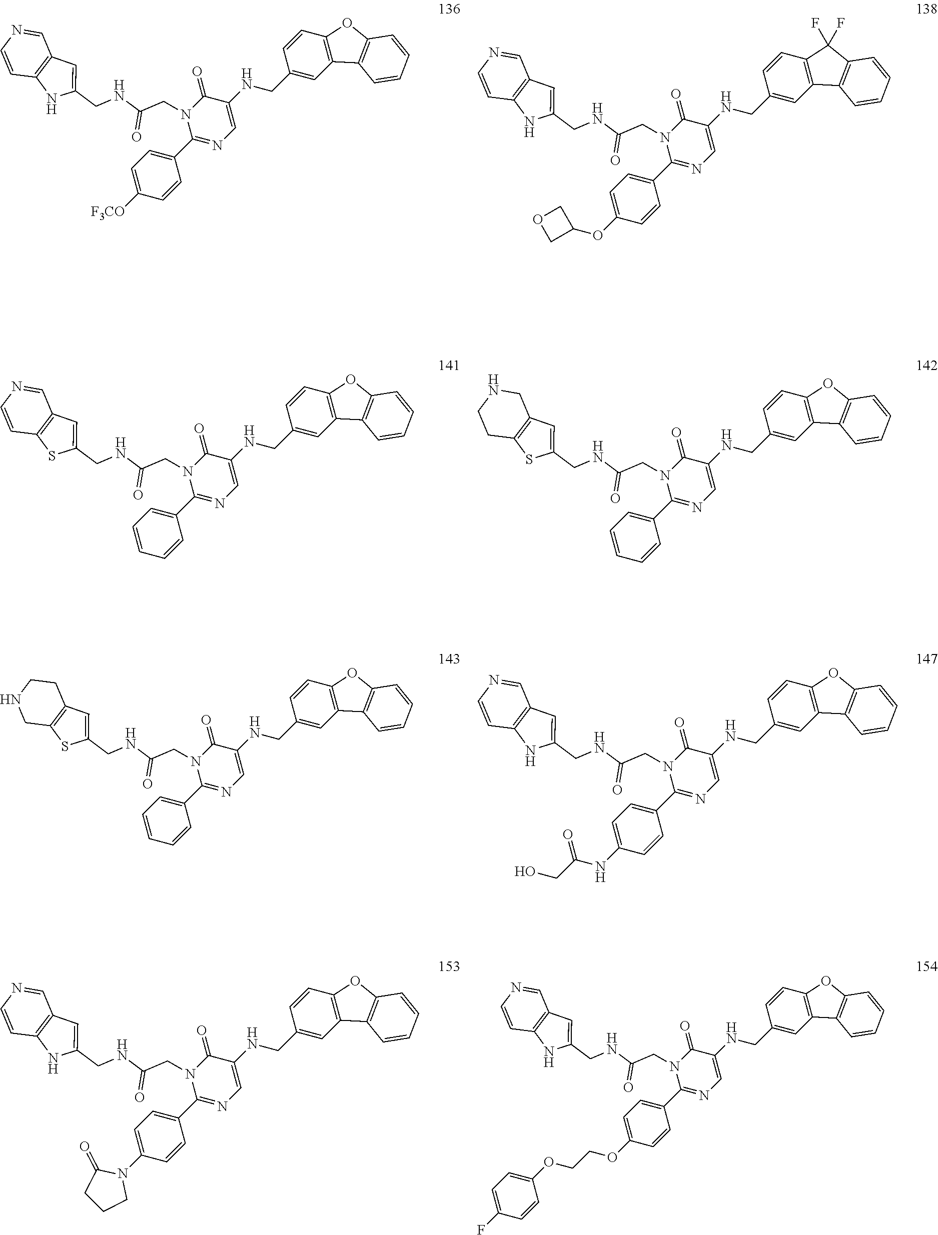 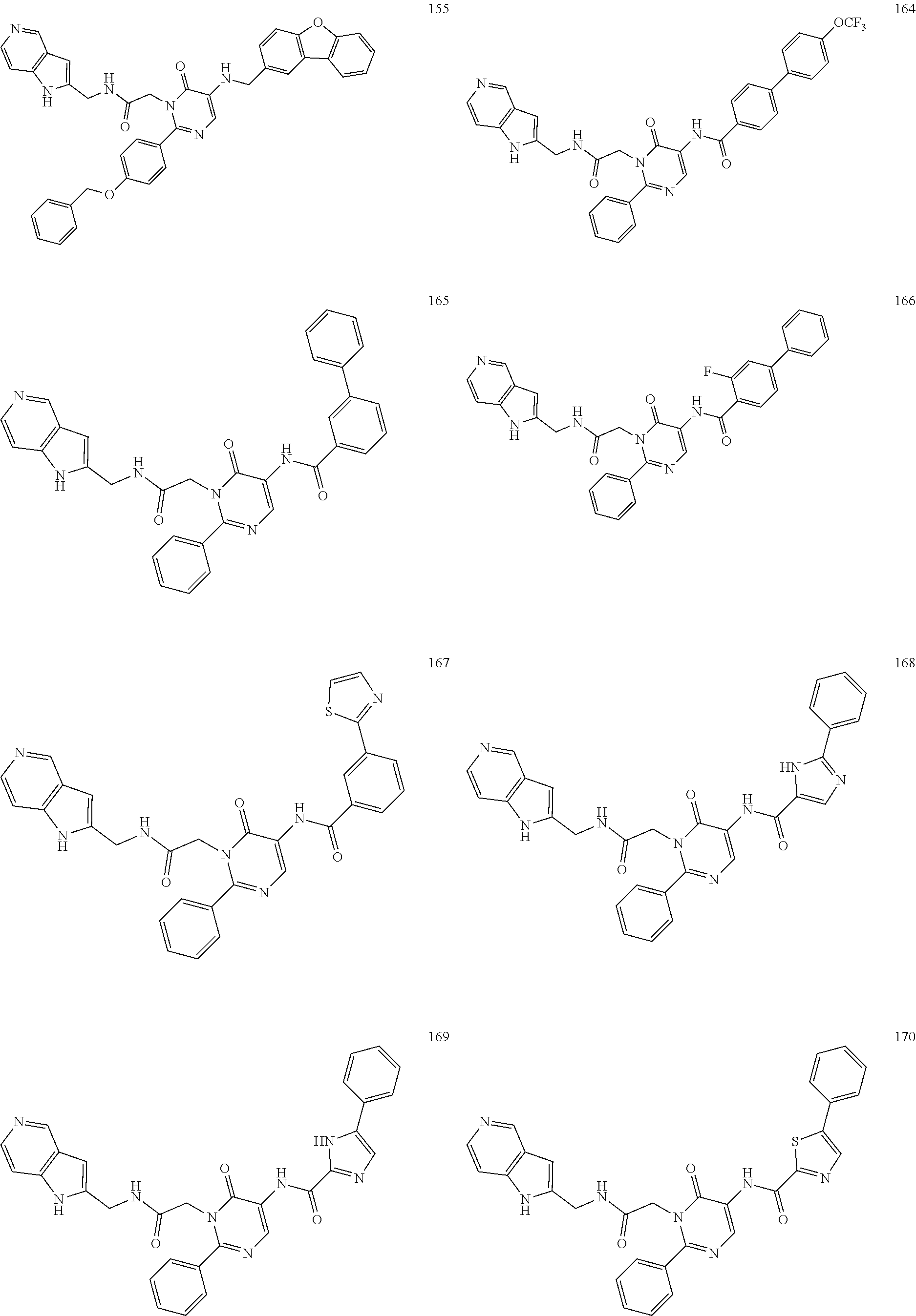 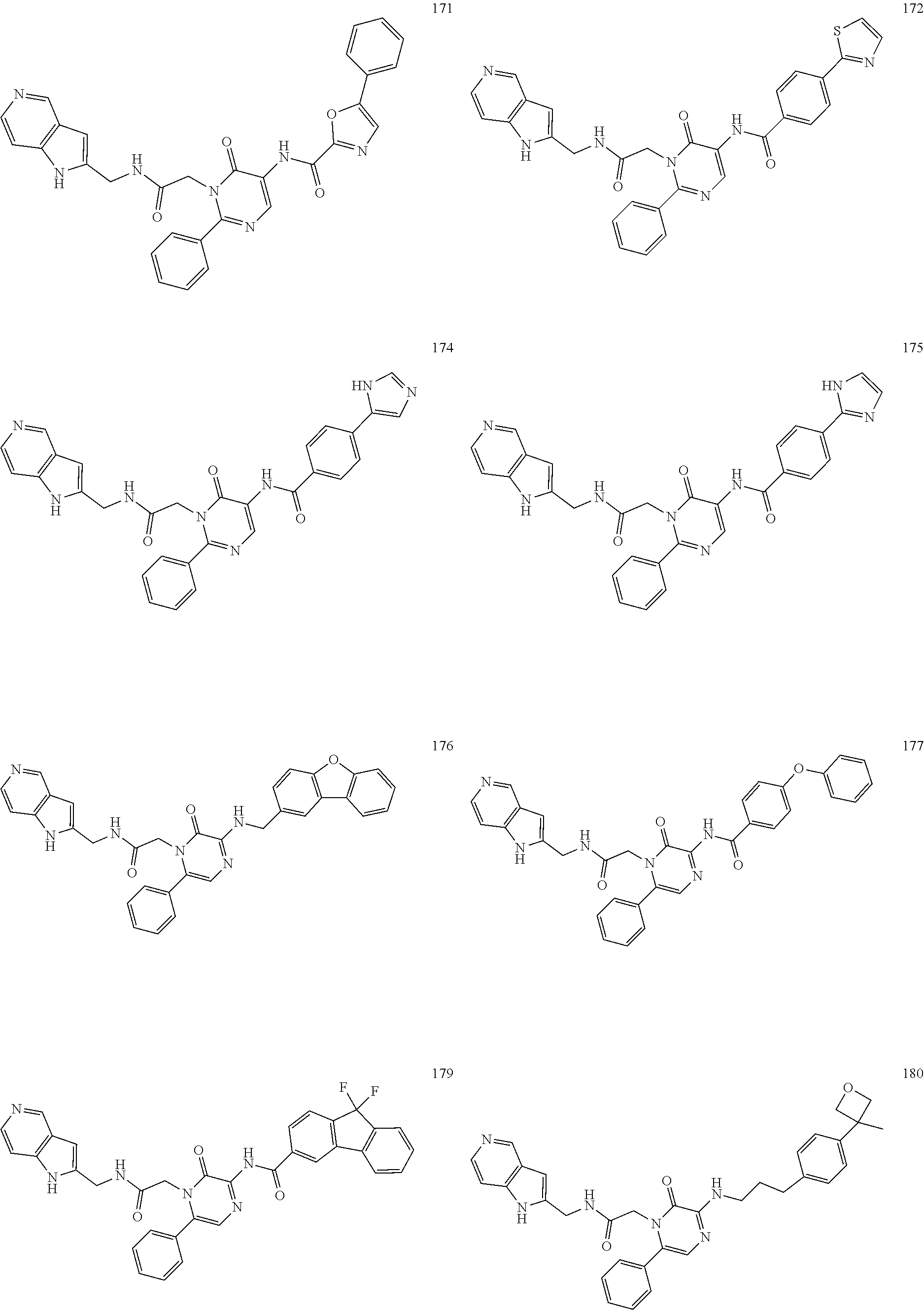 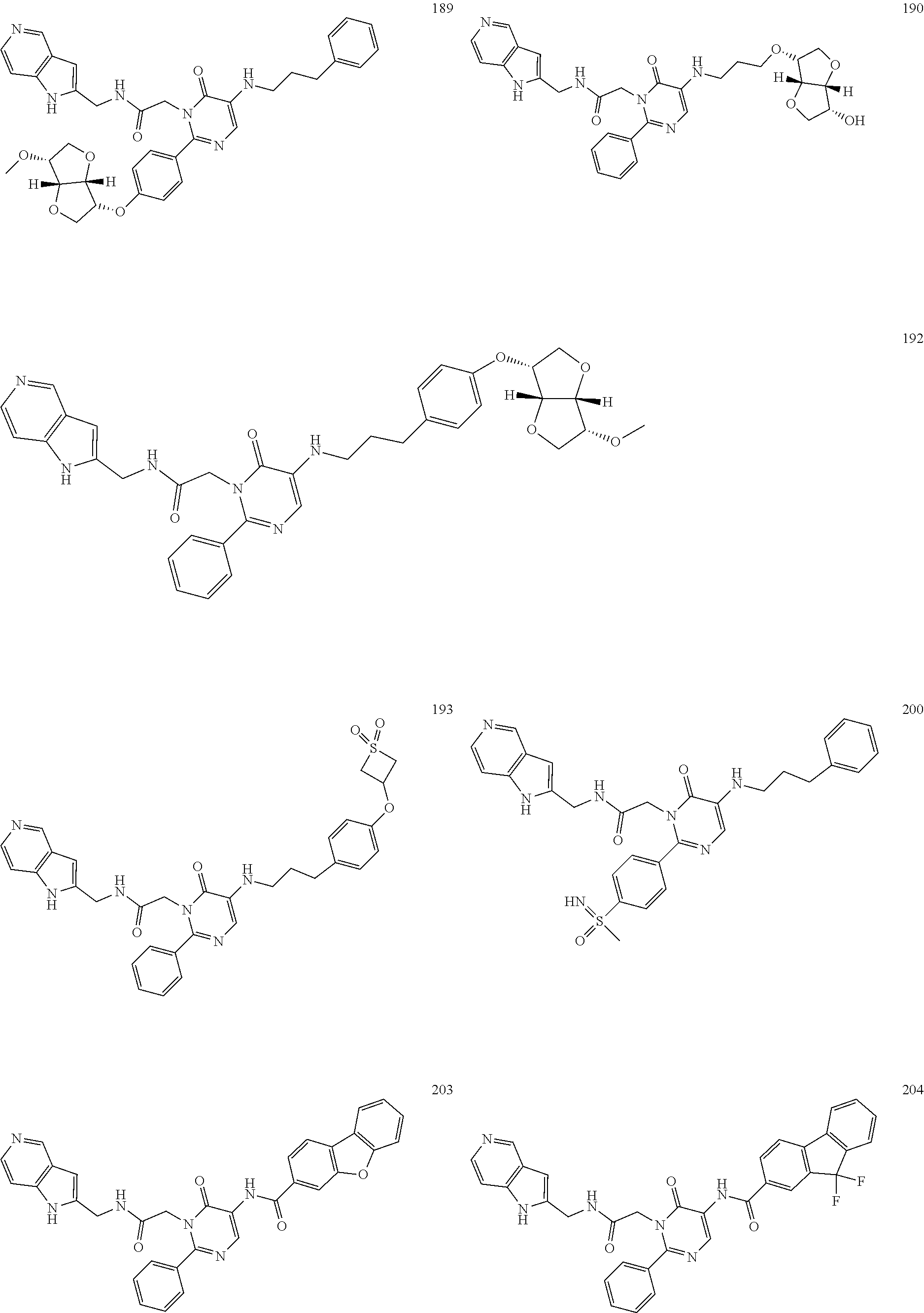 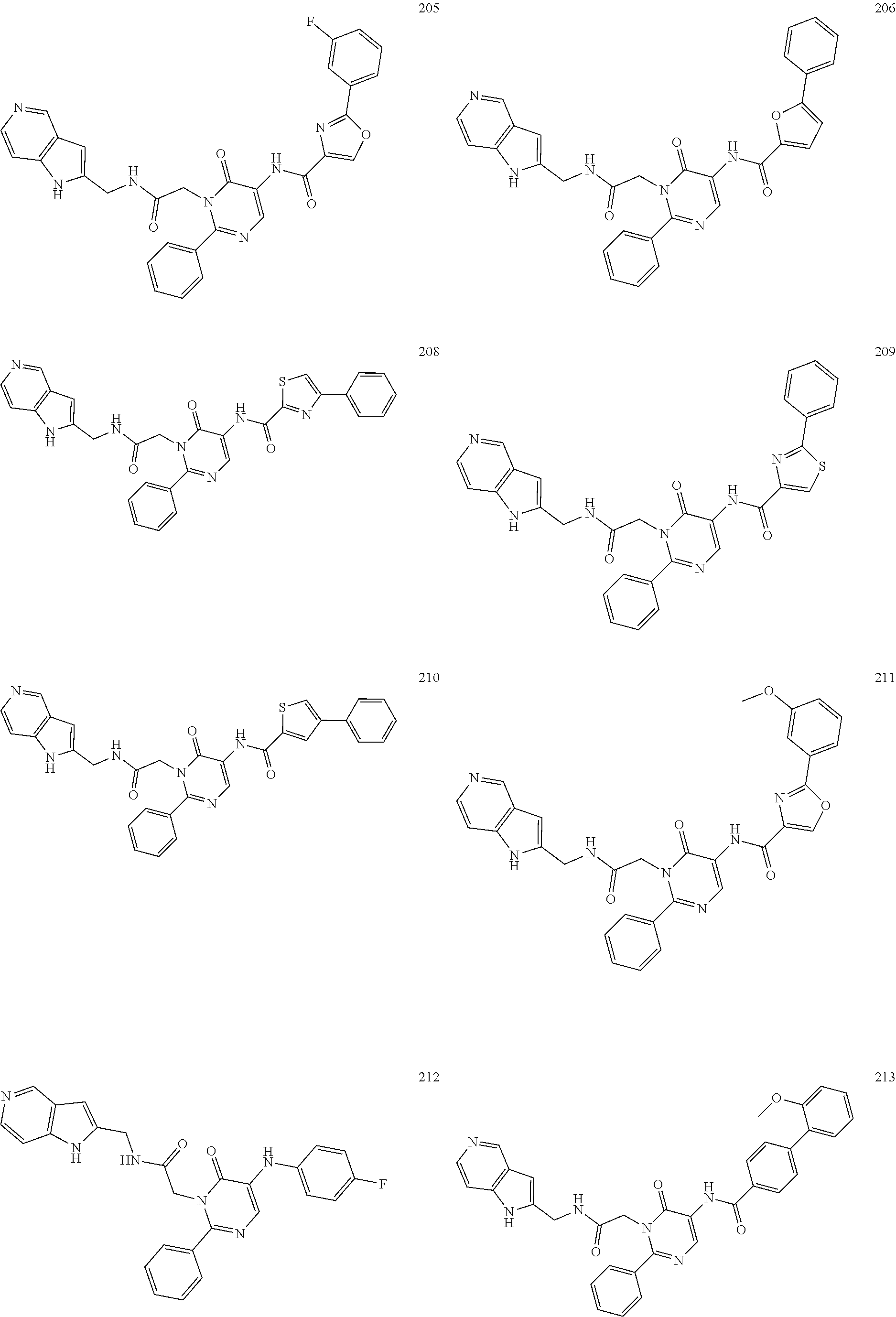 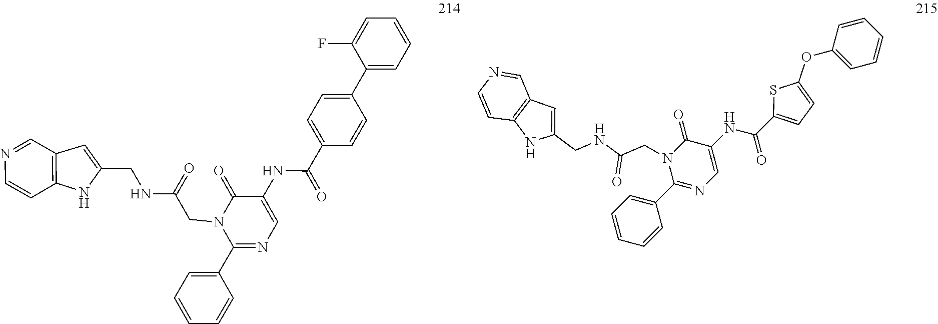 [SMILES: O=C(Cn1c(-c2ccccc2)ncc(NC(=O)c2ccc(-c3ccccc3F)cc2)c1=O)NCc1cc2cnccc2[nH]1 ; O=C(Cn1c(-c2ccccc2)ncc(NC(=O)c2ccc(Oc3ccccc3)s2)c1=O)NCc1cc2cnccc2[nH]1] 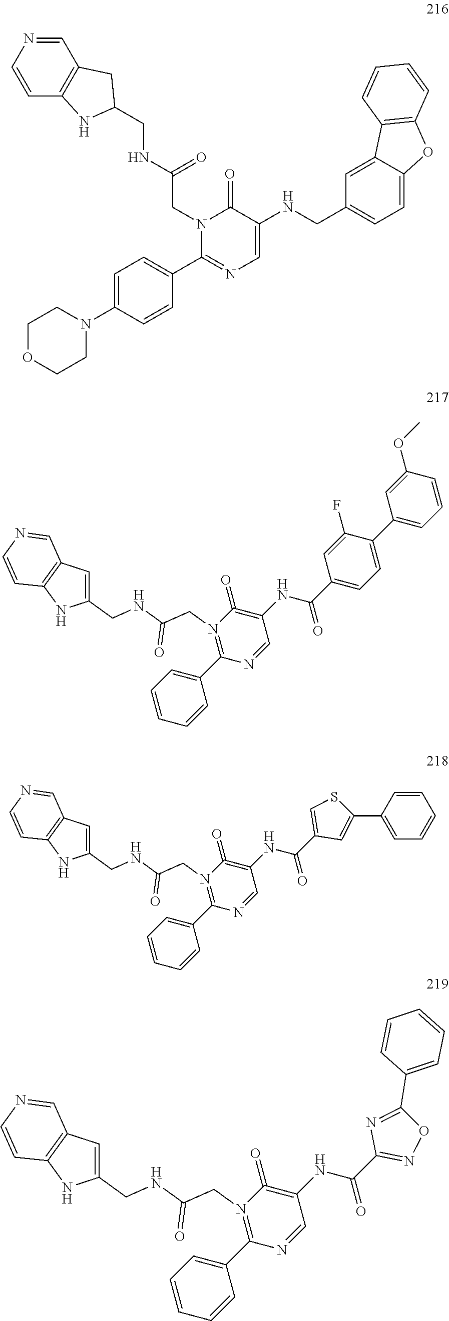 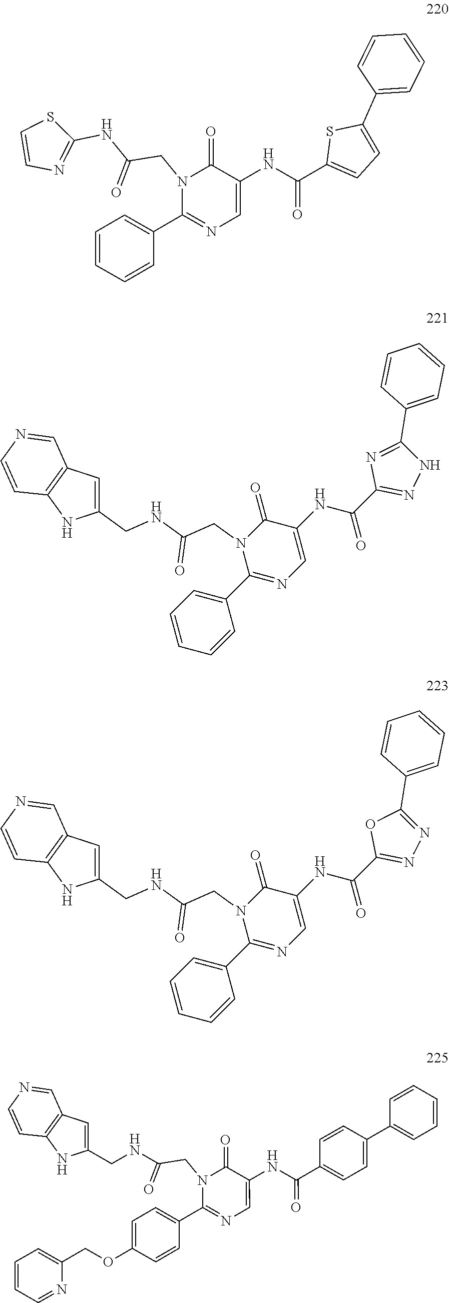 [SMILES: O=C(Cn1c(-c2ccccc2)ncc(NC(=O)c2ccc(-c3ccccc3)s2)c1=O)Nc1nccs1 ; O=C(Cn1c(-c2ccccc2)ncc(NC(=O)c2n[nH]c(-c3ccccc3)n2)c1=O)NCc1cc2cnccc2[nH]1 ; O=C(Cn1c(-c2ccccc2)ncc(NC(=O)c2nnc(-c3ccccc3)o2)c1=O)NCc1cc2cnccc2[nH]1 ; O=C(Cn1c(-c2ccc(OCc3ccccn3)cc2)ncc(NC(=O)c2ccc(-c3ccccc3)cc2)c1=O)NCc1cc2cnccc2[nH]1] 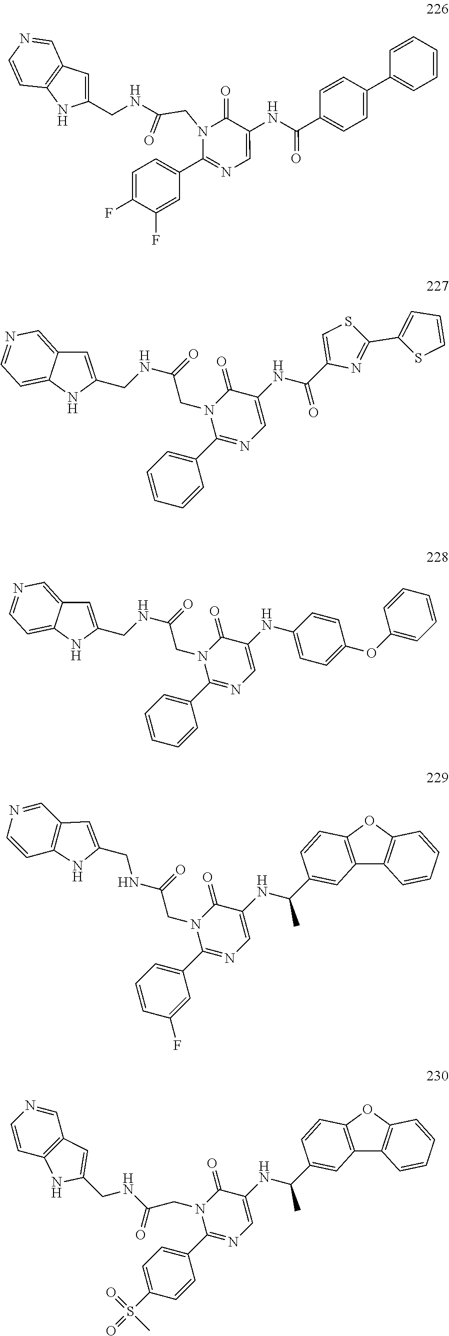 [SMILES: O=C(Cn1c(-c2ccc(F)c(F)c2)ncc(NC(=O)c2ccc(-c3ccccc3)cc2)c1=O)NCc1cc2cnccc2[nH]1 ; O=C(Cn1c(-c2ccccc2)ncc(NC(=O)c2csc(-c3cccs3)n2)c1=O)NCc1cc2cnccc2[nH]1 ; O=C(Cn1c(-c2ccccc2)ncc(Nc2ccc(Oc3ccccc3)cc2)c1=O)NCc1cc2cnccc2[nH]1 ; C[C@@H](Nc1cnc(-c2cccc(F)c2)n(CC(=O)NCc2cc3cnccc3[nH]2)c1=O)c1ccc2oc3ccccc3c2c1 ; C[C@@H](Nc1cnc(-c2ccc(S(C)(=O)=O)cc2)n(CC(=O)NCc2cc3cnccc3[nH]2)c1=O)c1ccc2oc3ccccc3c2c1] 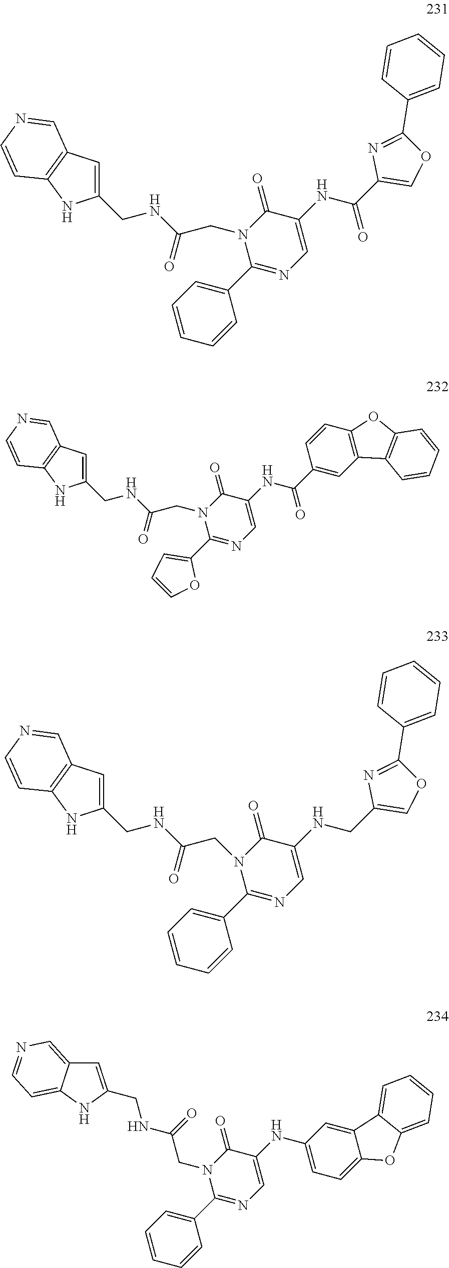 [SMILES: O=C(Cn1c(-c2ccccc2)ncc(NC(=O)c2coc(-c3ccccc3)n2)c1=O)NCc1cc2cnccc2[nH]1 ; O=C(Cn1c(-c2ccco2)ncc(NC(=O)c2ccc3oc4ccccc4c3c2)c1=O)NCc1cc2cnccc2[nH]1 ; O=C(Cn1c(-c2ccccc2)ncc(NCc2coc(-c3ccccc3)n2)c1=O)NCc1cc2cnccc2[nH]1 ; O=C(Cn1c(-c2ccccc2)ncc(Nc2ccc3oc4ccccc4c3c2)c1=O)NCc1cc2cnccc2[nH]1] 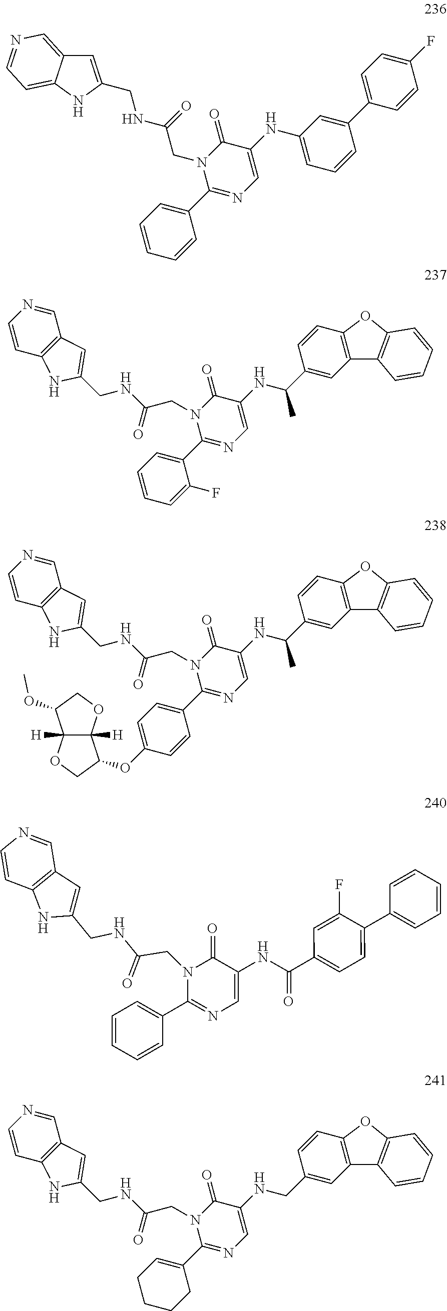 [SMILES: O=C(Cn1c(-c2ccccc2)ncc(Nc2cccc(-c3ccc(F)cc3)c2)c1=O)NCc1cc2cnccc2[nH]1 ; C[C@@H](Nc1cnc(-c2ccccc2F)n(CC(=O)NCc2cc3cnccc3[nH]2)c1=O)c1ccc2oc3ccccc3c2c1 ; CO[C@@H]1CO[C@H]2[C@@H]1OC[C@H]2Oc1ccc(-c2ncc(N[C@H](C)c3ccc4oc5ccccc5c4c3)c(=O)n2CC(=O)NCc2cc3cnccc3[nH]2)cc1 ; O=C(Cn1c(-c2ccccc2)ncc(NC(=O)c2ccc(-c3ccccc3)c(F)c2)c1=O)NCc1cc2cnccc2[nH]1 ; O=C(Cn1c(C2=CCCCC2)ncc(NCc2ccc3oc4ccccc4c3c2)c1=O)NCc1cc2cnccc2[nH]1] 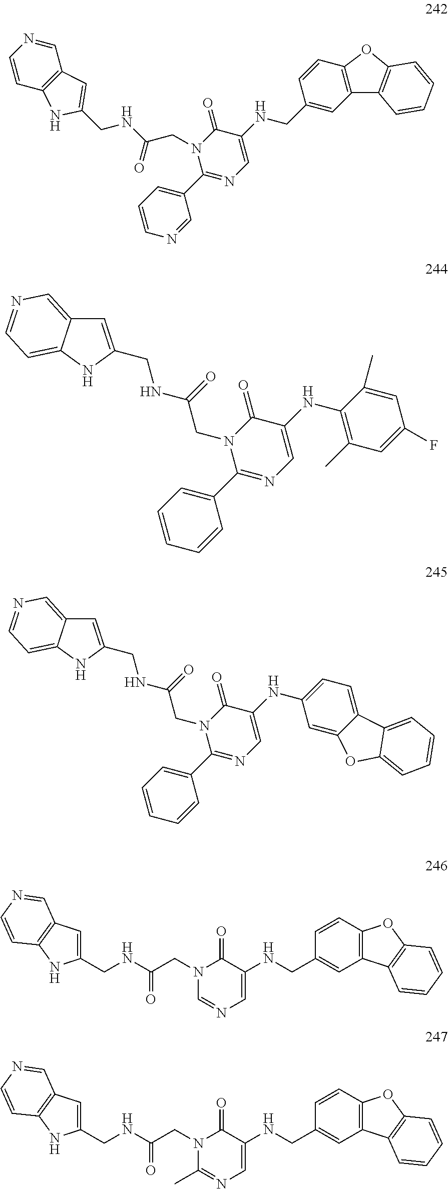 [SMILES: O=C(Cn1c(-c2cccnc2)ncc(NCc2ccc3oc4ccccc4c3c2)c1=O)NCc1cc2cnccc2[nH]1 ; Cc1cc(F)cc(C)c1Nc1cnc(-c2ccccc2)n(CC(=O)NCc2cc3cnccc3[nH]2)c1=O ; O=C(Cn1c(-c2ccccc2)ncc(Nc2ccc3c(c2)oc2ccccc23)c1=O)NCc1cc2cnccc2[nH]1 ; O=C(Cn1cncc(NCc2ccc3oc4ccccc4c3c2)c1=O)NCc1cc2cnccc2[nH]1 ; Cc1ncc(NCc2ccc3oc4ccccc4c3c2)c(=O)n1CC(=O)NCc1cc2cnccc2[nH]1] 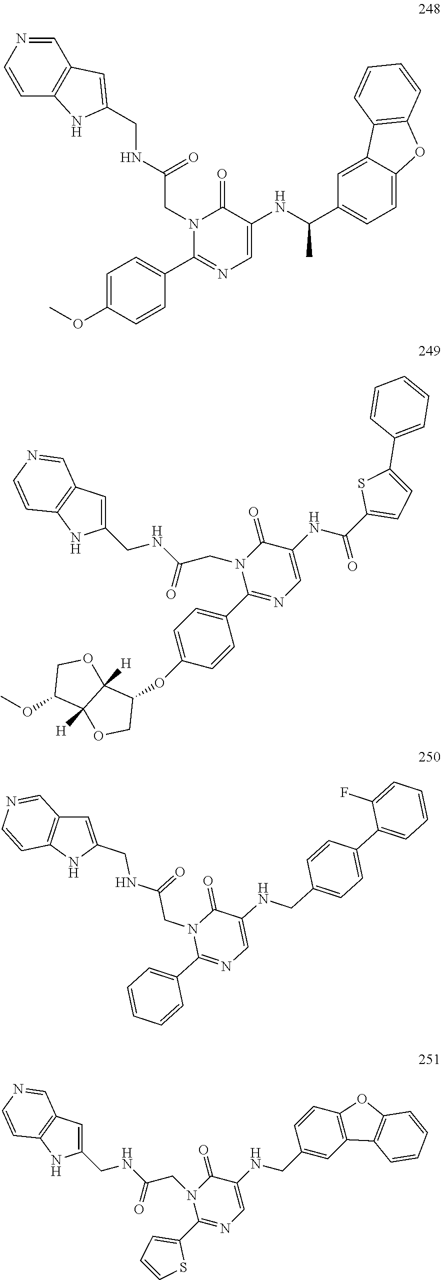 [SMILES: COc1ccc(-c2ncc(N[C@H](C)c3ccc4oc5ccccc5c4c3)c(=O)n2CC(=O)NCc2cc3cnccc3[nH]2)cc1 ; CO[C@@H]1CO[C@H]2[C@@H]1OC[C@H]2Oc1ccc(-c2ncc(NC(=O)c3ccc(-c4ccccc4)s3)c(=O)n2CC(=O)NCc2cc3cnccc3[nH]2)cc1 ; O=C(Cn1c(-c2ccccc2)ncc(NCc2ccc(-c3ccccc3F)cc2)c1=O)NCc1cc2cnccc2[nH]1 ; O=C(Cn1c(-c2cccs2)ncc(NCc2ccc3oc4ccccc4c3c2)c1=O)NCc1cc2cnccc2[nH]1] 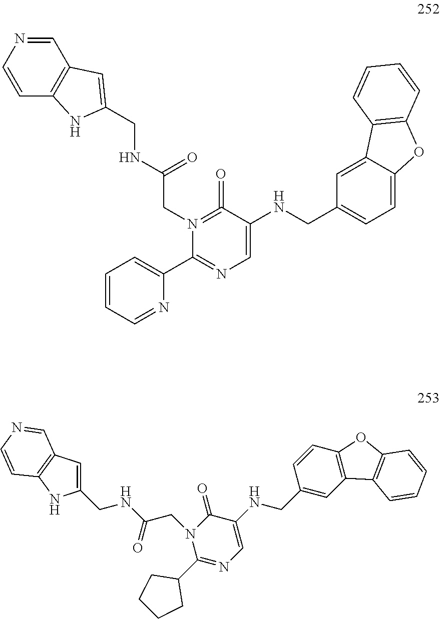 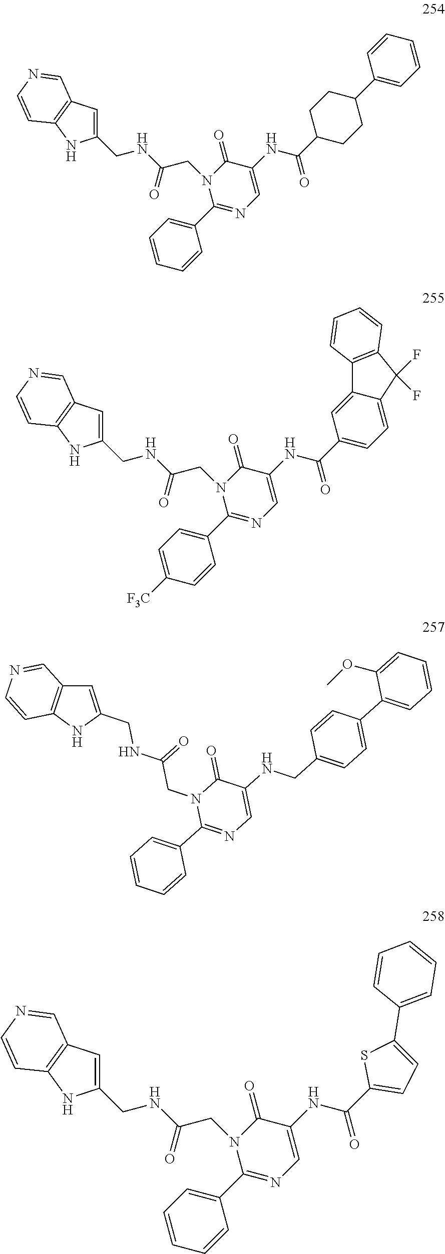 [SMILES: O=C(Cn1c(-c2ccccc2)ncc(NC(=O)C2CCC(c3ccccc3)CC2)c1=O)NCc1cc2cnccc2[nH]1 ; O=C(Cn1c(-c2ccc(C(F)(F)F)cc2)ncc(NC(=O)c2ccc3c(c2)-c2ccccc2C3(F)F)c1=O)NCc1cc2cnccc2[nH]1 ; COc1ccccc1-c1ccc(CNc2cnc(-c3ccccc3)n(CC(=O)NCc3cc4cnccc4[nH]3)c2=O)cc1 ; O=C(Cn1c(-c2ccccc2)ncc(NC(=O)c2ccc(-c3ccccc3)s2)c1=O)NCc1cc2cnccc2[nH]1] 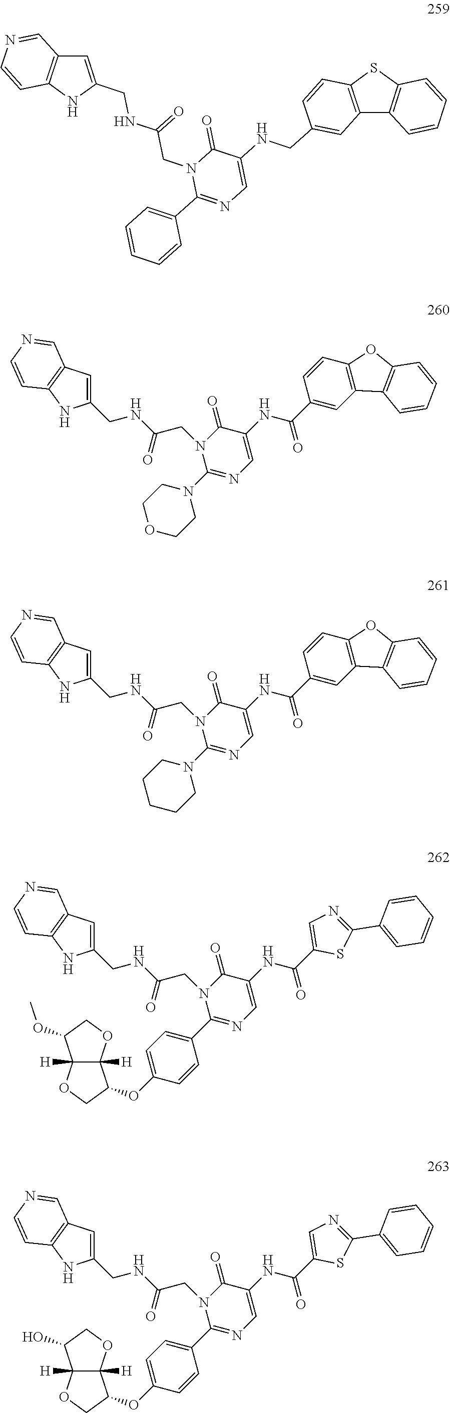 [SMILES: O=C(Cn1c(-c2ccccc2)ncc(NCc2ccc3sc4ccccc4c3c2)c1=O)NCc1cc2cnccc2[nH]1 ; O=C(Cn1c(N2CCOCC2)ncc(NC(=O)c2ccc3oc4ccccc4c3c2)c1=O)NCc1cc2cnccc2[nH]1 ; O=C(Cn1c(N2CCCCC2)ncc(NC(=O)c2ccc3oc4ccccc4c3c2)c1=O)NCc1cc2cnccc2[nH]1 ; CO[C@@H]1CO[C@H]2[C@@H]1OC[C@H]2Oc1ccc(-c2ncc(NC(=O)c3cnc(-c4ccccc4)s3)c(=O)n2CC(=O)NCc2cc3cnccc3[nH]2)cc1 ; O=C(Cn1c(-c2ccc(O[C@@H]3CO[C@H]4[C@@H]3OC[C@H]4O)cc2)ncc(NC(=O)c2cnc(-c3ccccc3)s2)c1=O)NCc1cc2cnccc2[nH]1] 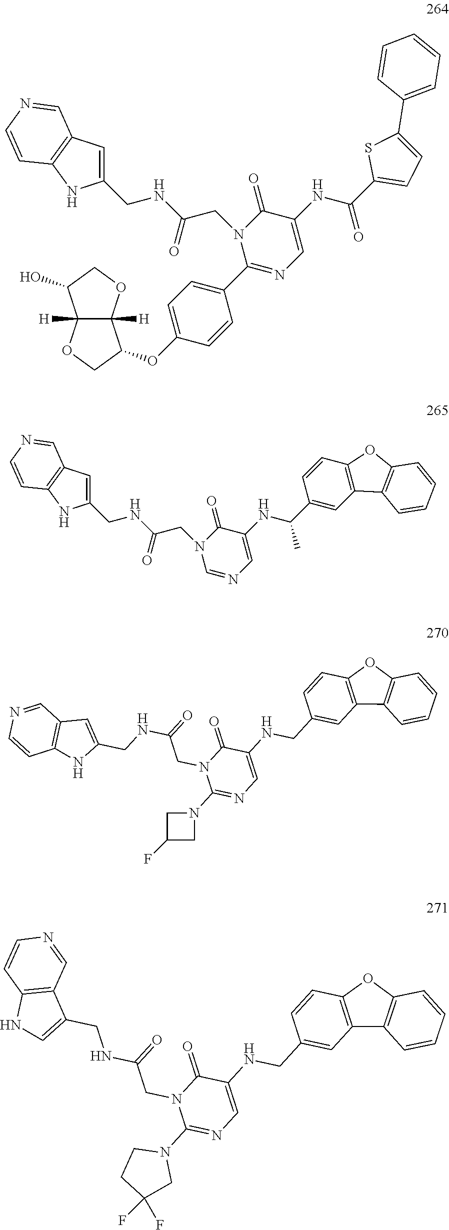 [SMILES: O=C(Cn1c(-c2ccc(O[C@@H]3CO[C@H]4[C@@H]3OC[C@H]4O)cc2)ncc(NC(=O)c2ccc(-c3ccccc3)s2)c1=O)NCc1cc2cnccc2[nH]1 ; C[C@H](Nc1cncn(CC(=O)NCc2cc3cnccc3[nH]2)c1=O)c1ccc2oc3ccccc3c2c1 ; O=C(Cn1c(N2CC(F)C2)ncc(NCc2ccc3oc4ccccc4c3c2)c1=O)NCc1cc2cnccc2[nH]1 ; O=C(Cn1c(N2CCC(F)(F)C2)ncc(NCc2ccc3oc4ccccc4c3c2)c1=O)NCc1c[nH]c2ccncc12] 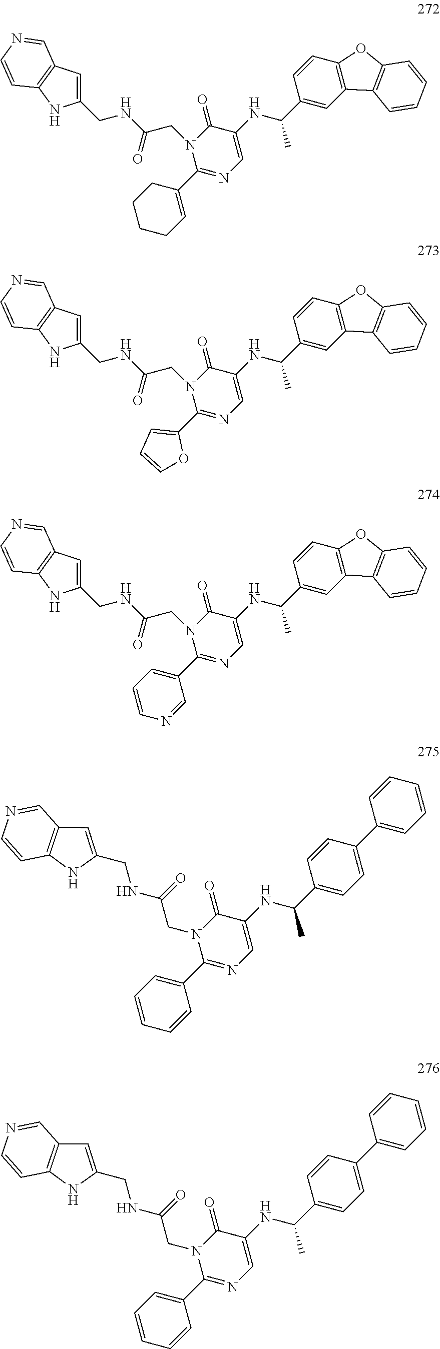 [SMILES: C[C@H](Nc1cnc(C2=CCCCC2)n(CC(=O)NCc2cc3cnccc3[nH]2)c1=O)c1ccc2oc3ccccc3c2c1 ; C[C@H](Nc1cnc(-c2ccco2)n(CC(=O)NCc2cc3cnccc3[nH]2)c1=O)c1ccc2oc3ccccc3c2c1 ; C[C@H](Nc1cnc(-c2cccnc2)n(CC(=O)NCc2cc3cnccc3[nH]2)c1=O)c1ccc2oc3ccccc3c2c1 ; C[C@@H](Nc1cnc(-c2ccccc2)n(CC(=O)NCc2cc3cnccc3[nH]2)c1=O)c1ccc(-c2ccccc2)cc1 ; C[C@H](Nc1cnc(-c2ccccc2)n(CC(=O)NCc2cc3cnccc3[nH]2)c1=O)c1ccc(-c2ccccc2)cc1] 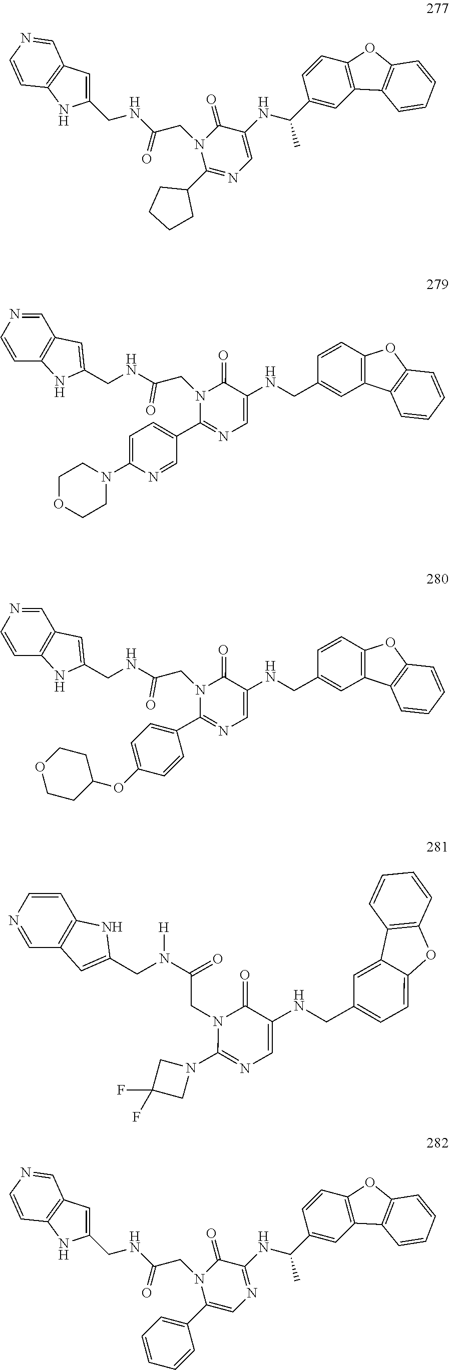 [SMILES: C[C@H](Nc1cnc(C2CCCC2)n(CC(=O)NCc2cc3cnccc3[nH]2)c1=O)c1ccc2oc3ccccc3c2c1 ; O=C(Cn1c(-c2ccc(N3CCOCC3)nc2)ncc(NCc2ccc3oc4ccccc4c3c2)c1=O)NCc1cc2cnccc2[nH]1 ; O=C(Cn1c(-c2ccc(OC3CCOCC3)cc2)ncc(NCc2ccc3oc4ccccc4c3c2)c1=O)NCc1cc2cnccc2[nH]1 ; O=C(Cn1c(N2CC(F)(F)C2)ncc(NCc2ccc3oc4ccccc4c3c2)c1=O)NCc1cc2cnccc2[nH]1 ; C[C@H](Nc1ncc(-c2ccccc2)n(CC(=O)NCc2cc3cnccc3[nH]2)c1=O)c1ccc2oc3ccccc3c2c1] 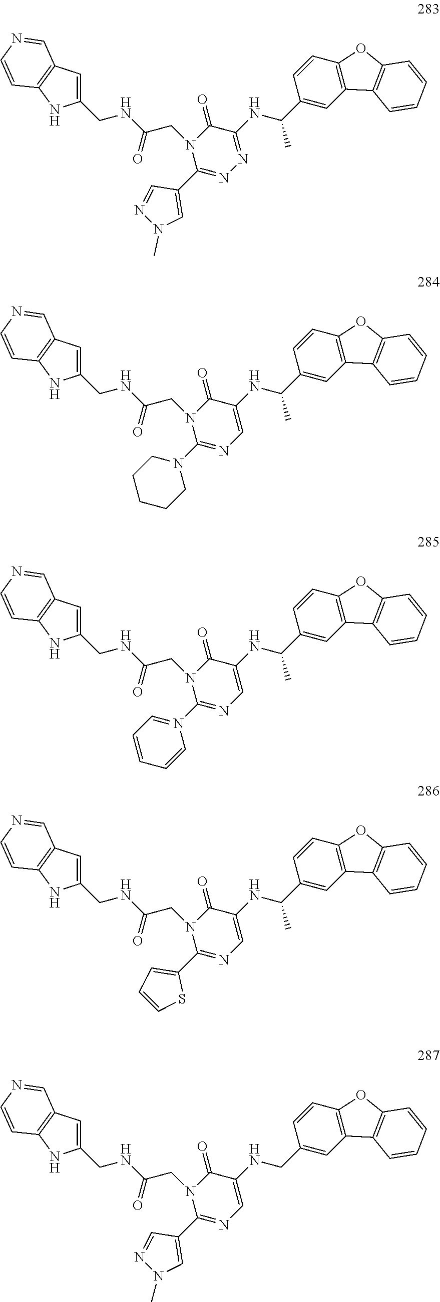 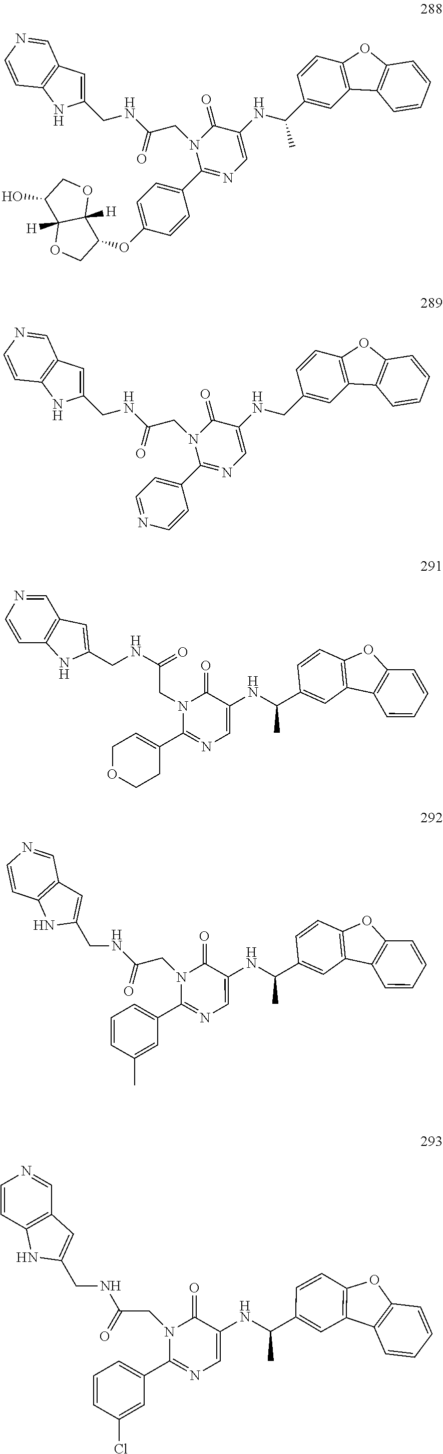 [SMILES: C[C@H](Nc1cnc(-c2ccc(O[C@@H]3CO[C@H]4[C@@H]3OC[C@H]4O)cc2)n(CC(=O)NCc2cc3cnccc3[nH]2)c1=O)c1ccc2oc3ccccc3c2c1 ; O=C(Cn1c(-c2ccncc2)ncc(NCc2ccc3oc4ccccc4c3c2)c1=O)NCc1cc2cnccc2[nH]1 ; C[C@@H](Nc1cnc(C2=CCOCC2)n(CC(=O)NCc2cc3cnccc3[nH]2)c1=O)c1ccc2oc3ccccc3c2c1 ; Cc1cccc(-c2ncc(N[C@H](C)c3ccc4oc5ccccc5c4c3)c(=O)n2CC(=O)NCc2cc3cnccc3[nH]2)c1 ; C[C@@H](Nc1cnc(-c2cccc(Cl)c2)n(CC(=O)NCc2cc3cnccc3[nH]2)c1=O)c1ccc2oc3ccccc3c2c1] 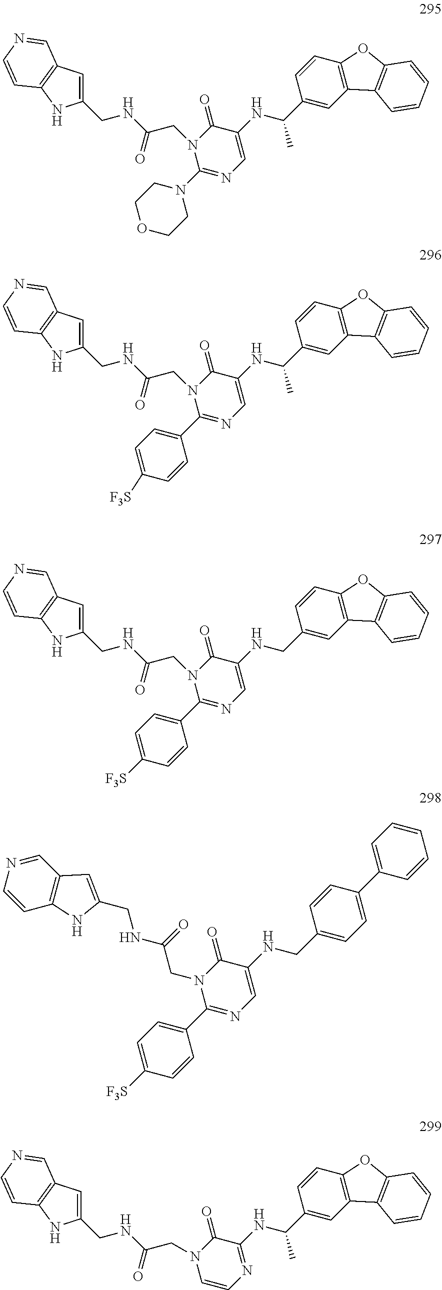 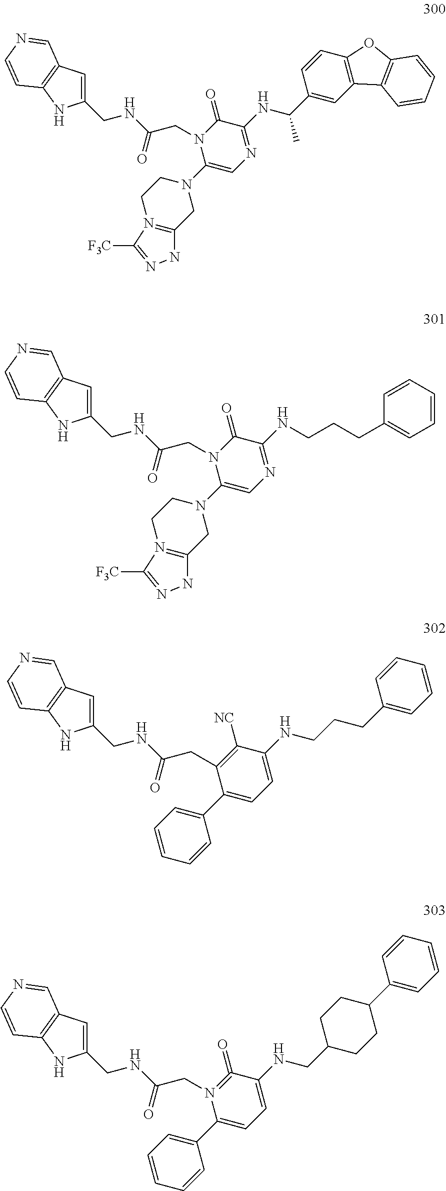 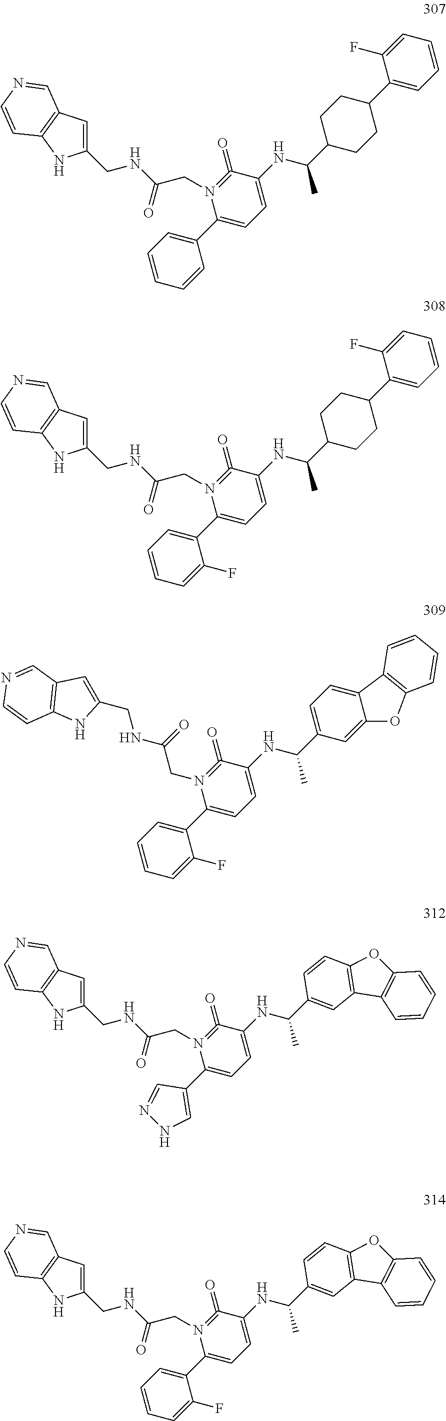 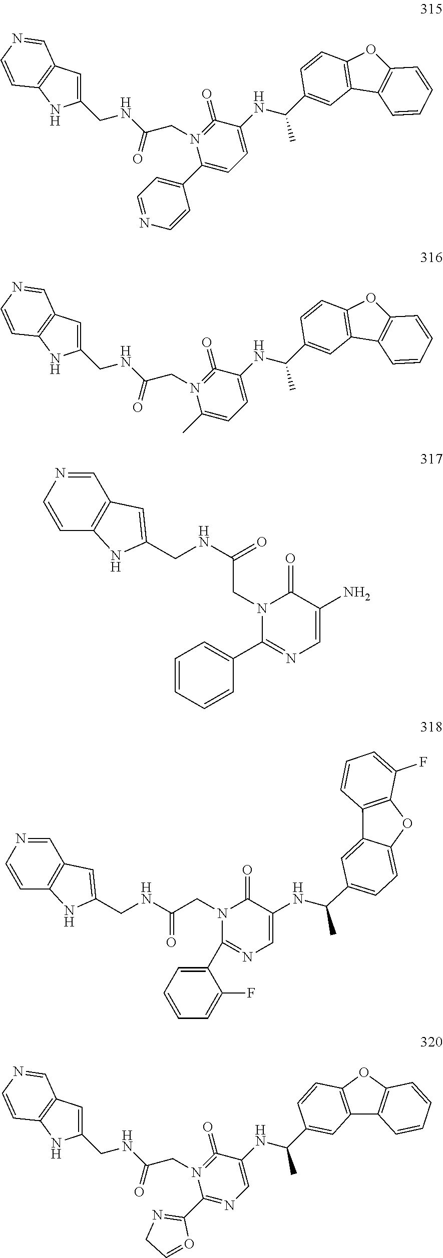 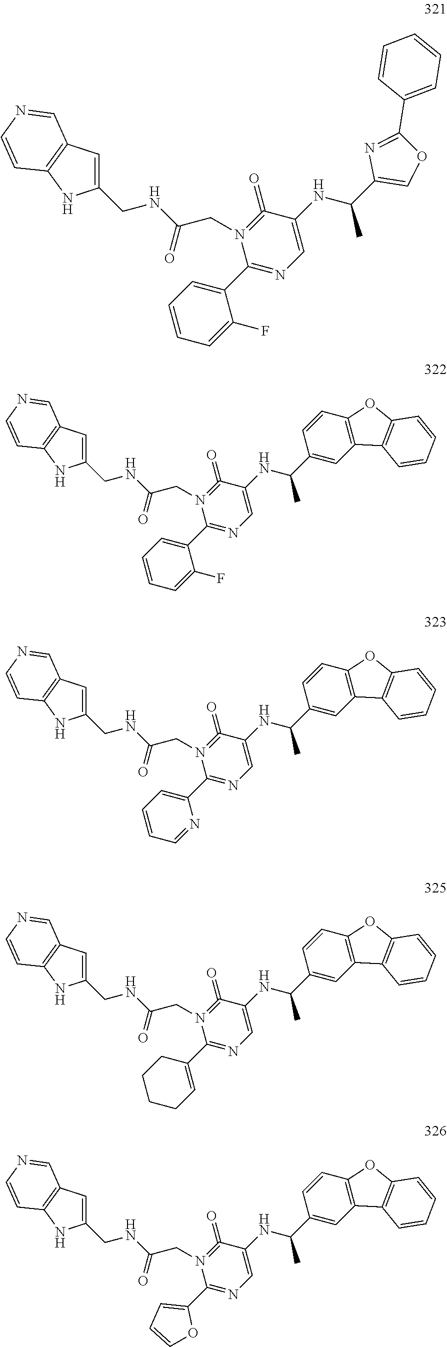 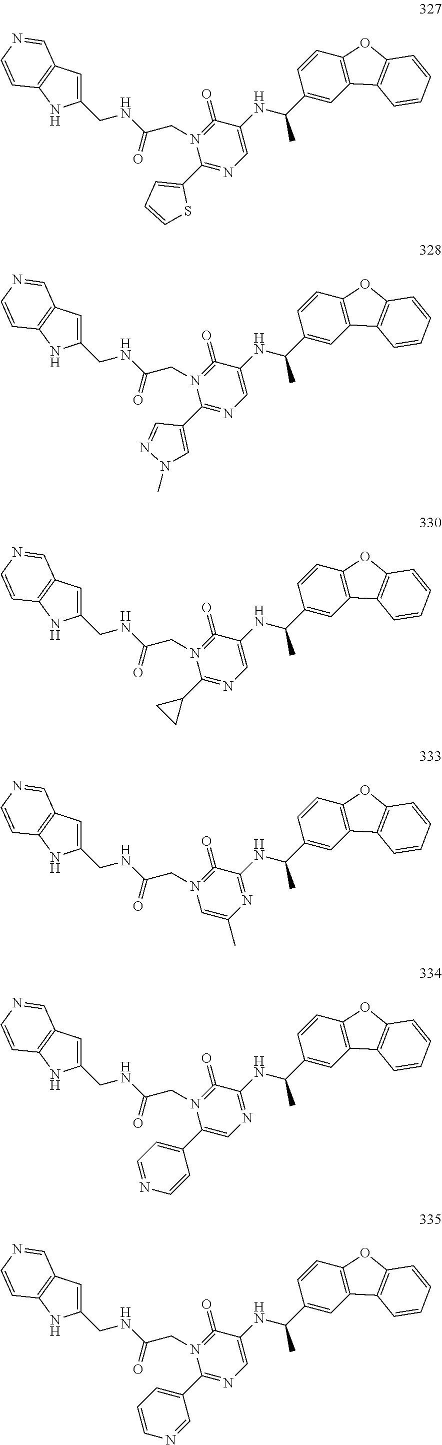 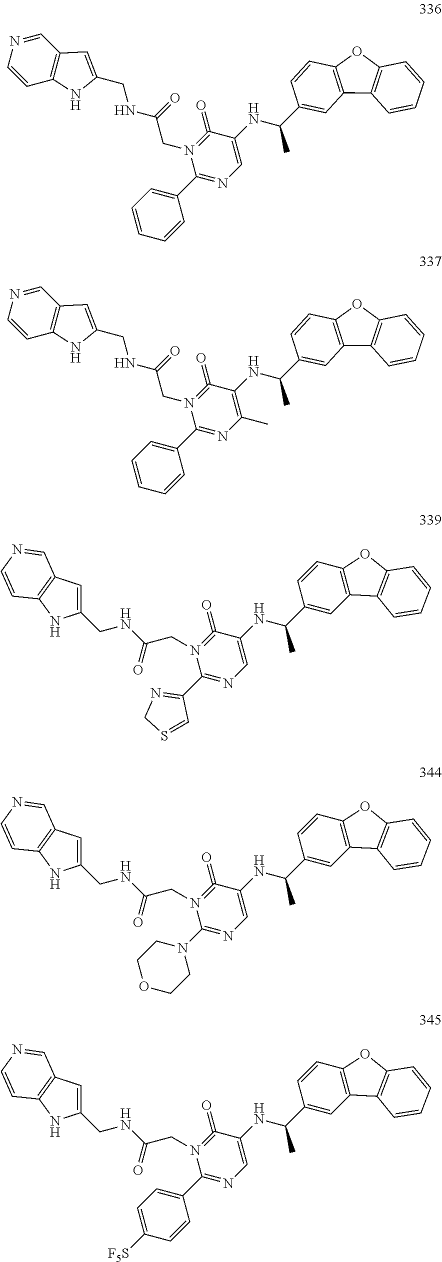 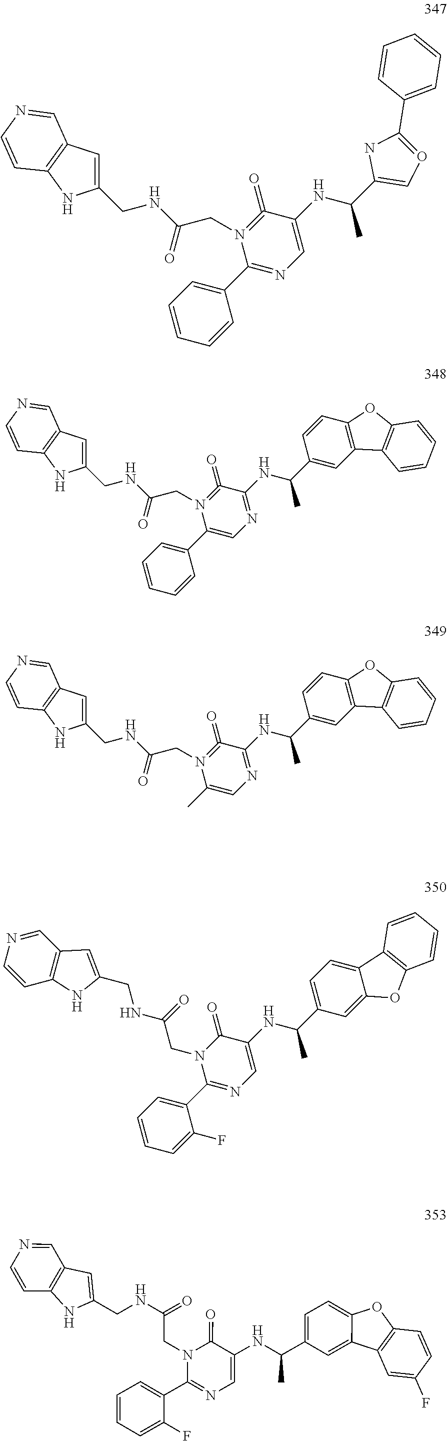 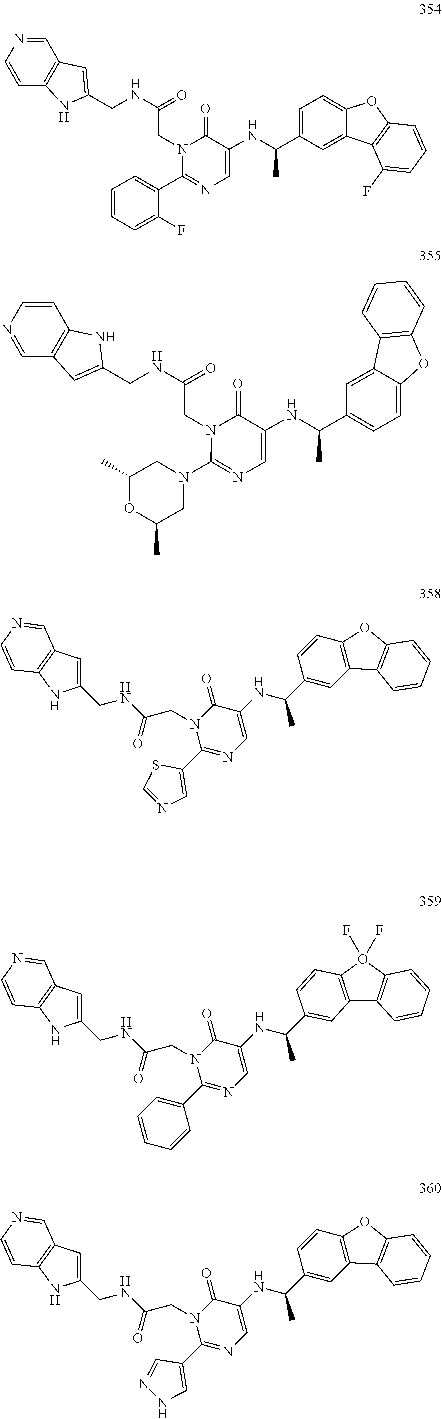 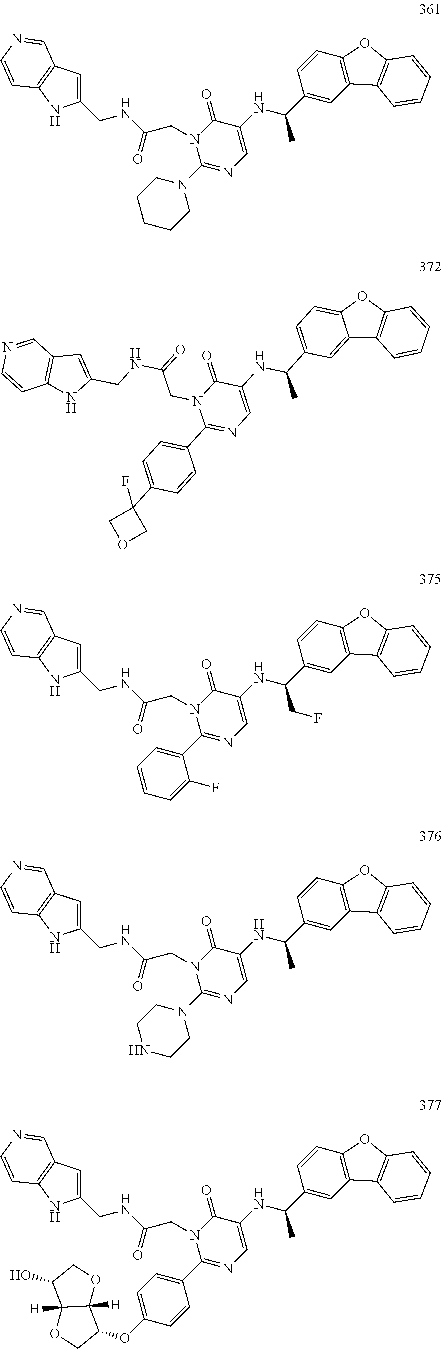 [SMILES: C[C@@H](Nc1cnc(N2CCCCC2)n(CC(=O)NCc2cc3cnccc3[nH]2)c1=O)c1ccc2oc3ccccc3c2c1 ; C[C@@H](Nc1cnc(-c2ccc(C3(F)COC3)cc2)n(CC(=O)NCc2cc3cnccc3[nH]2)c1=O)c1ccc2oc3ccccc3c2c1 ; O=C(Cn1c(-c2ccccc2F)ncc(N[C@H](CF)c2ccc3oc4ccccc4c3c2)c1=O)NCc1cc2cnccc2[nH]1 ; C[C@@H](Nc1cnc(N2CCNCC2)n(CC(=O)NCc2cc3cnccc3[nH]2)c1=O)c1ccc2oc3ccccc3c2c1 ; C[C@@H](Nc1cnc(-c2ccc(O[C@@H]3CO[C@H]4[C@@H]3OC[C@H]4O)cc2)n(CC(=O)NCc2cc3cnccc3[nH]2)c1=O)c1ccc2oc3ccccc3c2c1] 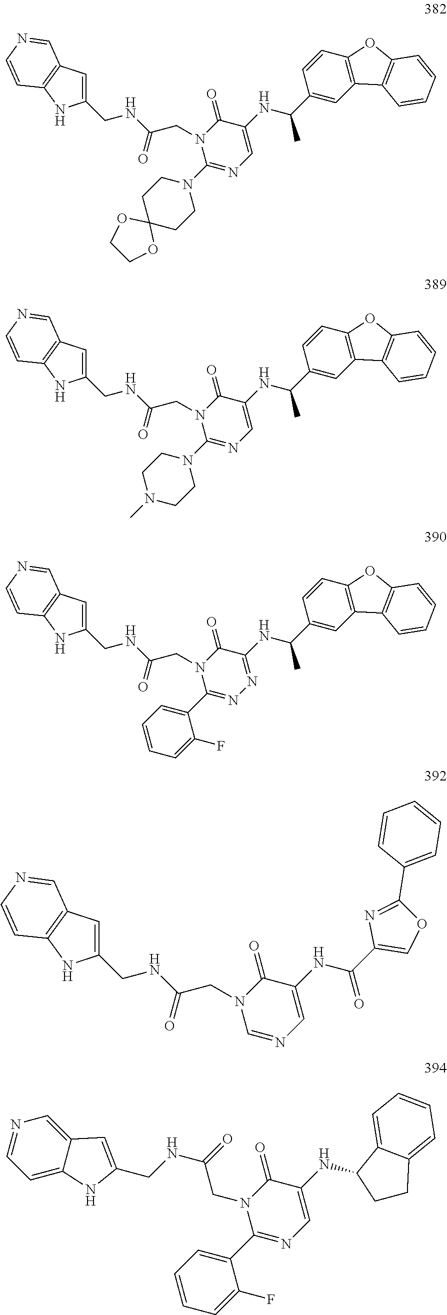 [SMILES: C[C@@H](Nc1cnc(N2CCC3(CC2)OCCO3)n(CC(=O)NCc2cc3cnccc3[nH]2)c1=O)c1ccc2oc3ccccc3c2c1 ; C[C@@H](Nc1cnc(N2CCN(C)CC2)n(CC(=O)NCc2cc3cnccc3[nH]2)c1=O)c1ccc2oc3ccccc3c2c1 ; C[C@@H](Nc1nnc(-c2ccccc2F)n(CC(=O)NCc2cc3cnccc3[nH]2)c1=O)c1ccc2oc3ccccc3c2c1 ; O=C(Cn1cncc(NC(=O)c2coc(-c3ccccc3)n2)c1=O)NCc1cc2cnccc2[nH]1 ; O=C(Cn1c(-c2ccccc2F)ncc(N[C@H]2CCc3ccccc32)c1=O)NCc1cc2cnccc2[nH]1] 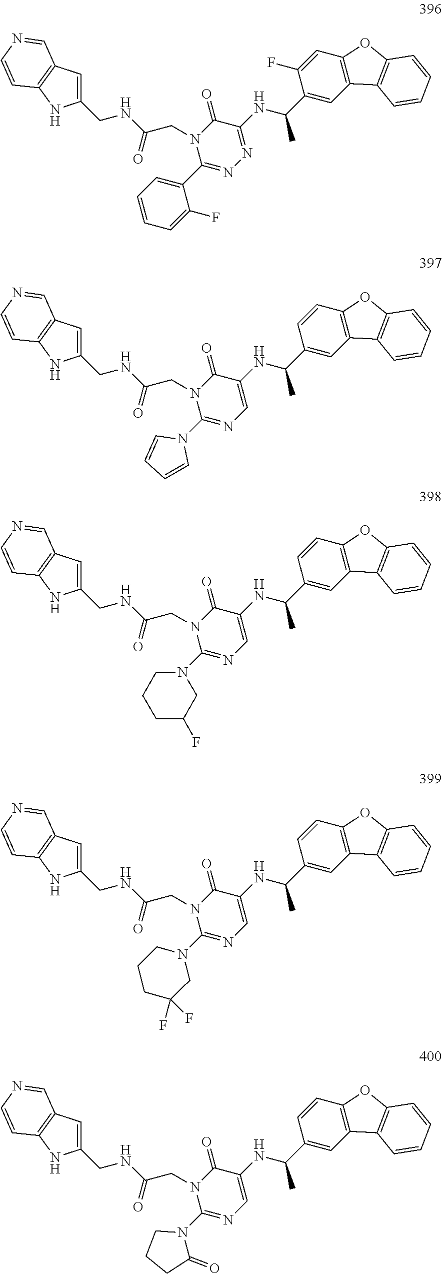 [SMILES: C[C@@H](Nc1nnc(-c2ccccc2F)n(CC(=O)NCc2cc3cnccc3[nH]2)c1=O)c1cc2c(cc1F)oc1ccccc12 ; C[C@@H](Nc1cnc(-n2cccc2)n(CC(=O)NCc2cc3cnccc3[nH]2)c1=O)c1ccc2oc3ccccc3c2c1 ; C[C@@H](Nc1cnc(N2CCCC(F)C2)n(CC(=O)NCc2cc3cnccc3[nH]2)c1=O)c1ccc2oc3ccccc3c2c1 ; C[C@@H](Nc1cnc(N2CCCC(F)(F)C2)n(CC(=O)NCc2cc3cnccc3[nH]2)c1=O)c1ccc2oc3ccccc3c2c1 ; C[C@@H](Nc1cnc(N2CCCC2=O)n(CC(=O)NCc2cc3cnccc3[nH]2)c1=O)c1ccc2oc3ccccc3c2c1] 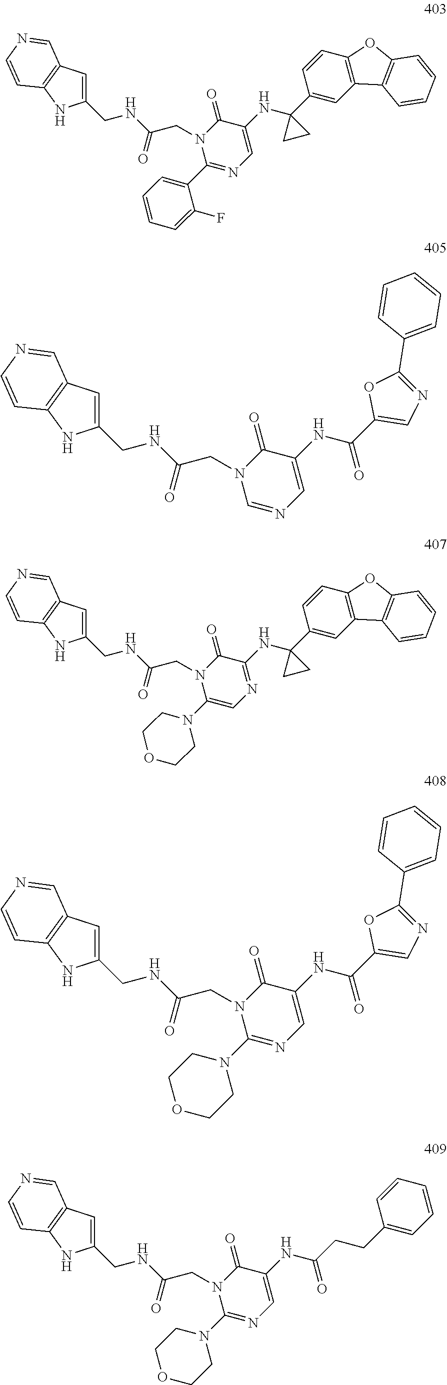 [SMILES: O=C(Cn1c(-c2ccccc2F)ncc(NC2(c3ccc4oc5ccccc5c4c3)CC2)c1=O)NCc1cc2cnccc2[nH]1 ; O=C(Cn1cncc(NC(=O)c2cnc(-c3ccccc3)o2)c1=O)NCc1cc2cnccc2[nH]1 ; O=C(Cn1c(N2CCOCC2)cnc(NC2(c3ccc4oc5ccccc5c4c3)CC2)c1=O)NCc1cc2cnccc2[nH]1 ; O=C(Cn1c(N2CCOCC2)ncc(NC(=O)c2cnc(-c3ccccc3)o2)c1=O)NCc1cc2cnccc2[nH]1 ; O=C(Cn1c(N2CCOCC2)ncc(NC(=O)CCc2ccccc2)c1=O)NCc1cc2cnccc2[nH]1] 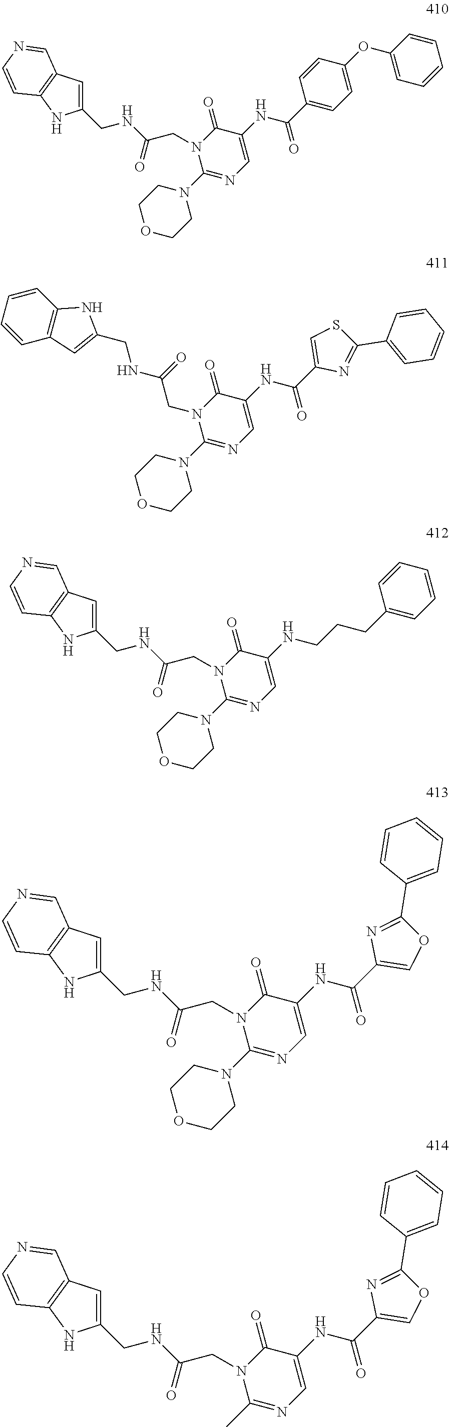 [SMILES: O=C(Cn1c(N2CCOCC2)ncc(NC(=O)c2ccc(Oc3ccccc3)cc2)c1=O)NCc1cc2cnccc2[nH]1 ; O=C(Cn1c(N2CCOCC2)ncc(NC(=O)c2csc(-c3ccccc3)n2)c1=O)NCc1cc2ccccc2[nH]1 ; O=C(Cn1c(N2CCOCC2)ncc(NCCCc2ccccc2)c1=O)NCc1cc2cnccc2[nH]1 ; O=C(Cn1c(N2CCOCC2)ncc(NC(=O)c2coc(-c3ccccc3)n2)c1=O)NCc1cc2cnccc2[nH]1 ; Cc1ncc(NC(=O)c2coc(-c3ccccc3)n2)c(=O)n1CC(=O)NCc1cc2cnccc2[nH]1] 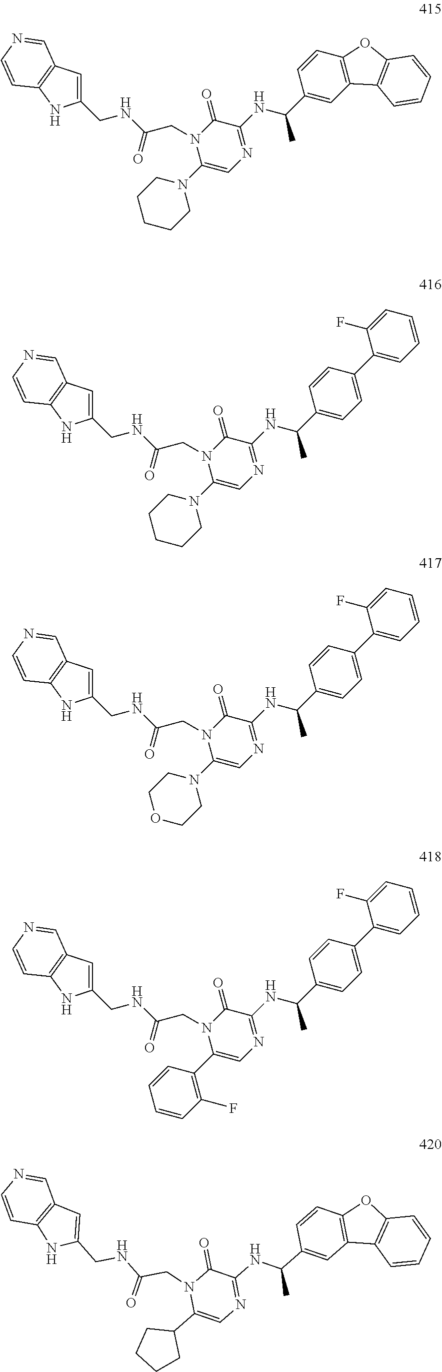 [SMILES: C[C@@H](Nc1ncc(N2CCCCC2)n(CC(=O)NCc2cc3cnccc3[nH]2)c1=O)c1ccc2oc3ccccc3c2c1 ; C[C@@H](Nc1ncc(N2CCCCC2)n(CC(=O)NCc2cc3cnccc3[nH]2)c1=O)c1ccc(-c2ccccc2F)cc1 ; C[C@@H](Nc1ncc(N2CCOCC2)n(CC(=O)NCc2cc3cnccc3[nH]2)c1=O)c1ccc(-c2ccccc2F)cc1 ; C[C@@H](Nc1ncc(-c2ccccc2F)n(CC(=O)NCc2cc3cnccc3[nH]2)c1=O)c1ccc(-c2ccccc2F)cc1 ; C[C@@H](Nc1ncc(C2CCCC2)n(CC(=O)NCc2cc3cnccc3[nH]2)c1=O)c1ccc2oc3ccccc3c2c1] 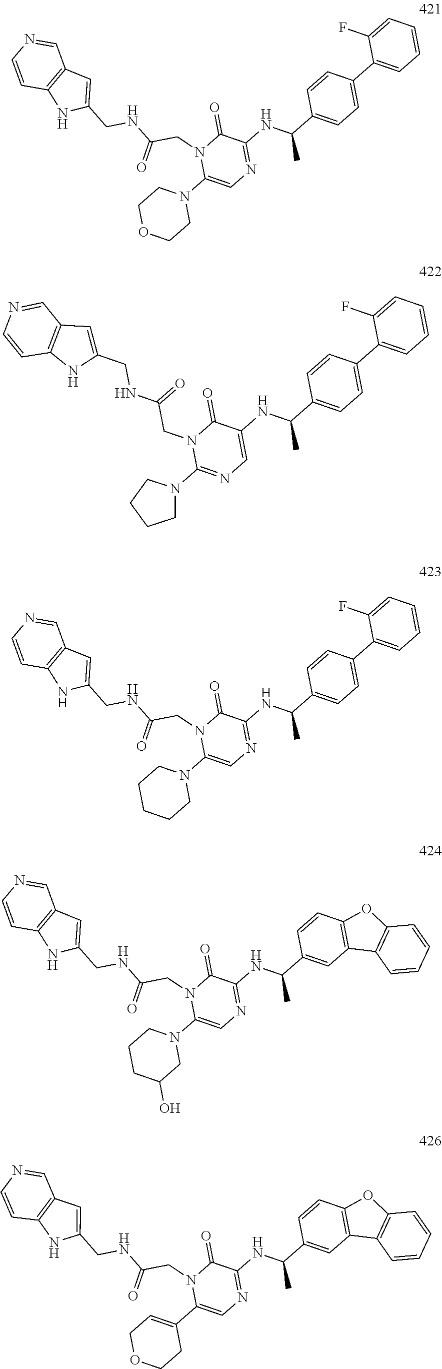 [SMILES: C[C@@H](Nc1ncc(N2CCOCC2)n(CC(=O)NCc2cc3cnccc3[nH]2)c1=O)c1ccc(-c2ccccc2F)cc1 ; C[C@@H](Nc1cnc(N2CCCC2)n(CC(=O)NCc2cc3cnccc3[nH]2)c1=O)c1ccc(-c2ccccc2F)cc1 ; C[C@@H](Nc1ncc(N2CCCCC2)n(CC(=O)NCc2cc3cnccc3[nH]2)c1=O)c1ccc(-c2ccccc2F)cc1 ; C[C@@H](Nc1ncc(N2CCCC(O)C2)n(CC(=O)NCc2cc3cnccc3[nH]2)c1=O)c1ccc2oc3ccccc3c2c1 ; C[C@@H](Nc1ncc(C2=CCOCC2)n(CC(=O)NCc2cc3cnccc3[nH]2)c1=O)c1ccc2oc3ccccc3c2c1] 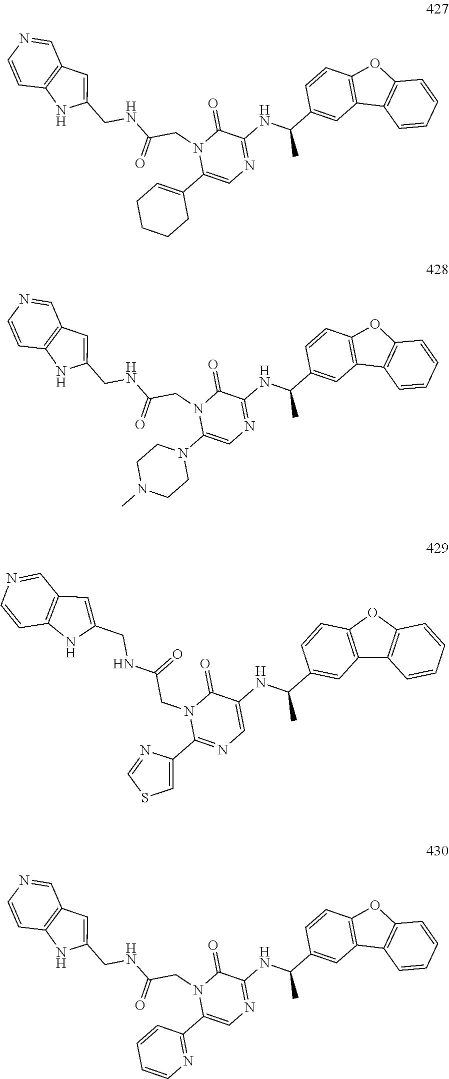 [SMILES: C[C@@H](Nc1ncc(C2=CCCCC2)n(CC(=O)NCc2cc3cnccc3[nH]2)c1=O)c1ccc2oc3ccccc3c2c1 ; C[C@@H](Nc1ncc(N2CCN(C)CC2)n(CC(=O)NCc2cc3cnccc3[nH]2)c1=O)c1ccc2oc3ccccc3c2c1 ; C[C@@H](Nc1cnc(-c2cscn2)n(CC(=O)NCc2cc3cnccc3[nH]2)c1=O)c1ccc2oc3ccccc3c2c1 ; C[C@@H](Nc1ncc(-c2ccccn2)n(CC(=O)NCc2cc3cnccc3[nH]2)c1=O)c1ccc2oc3ccccc3c2c1] 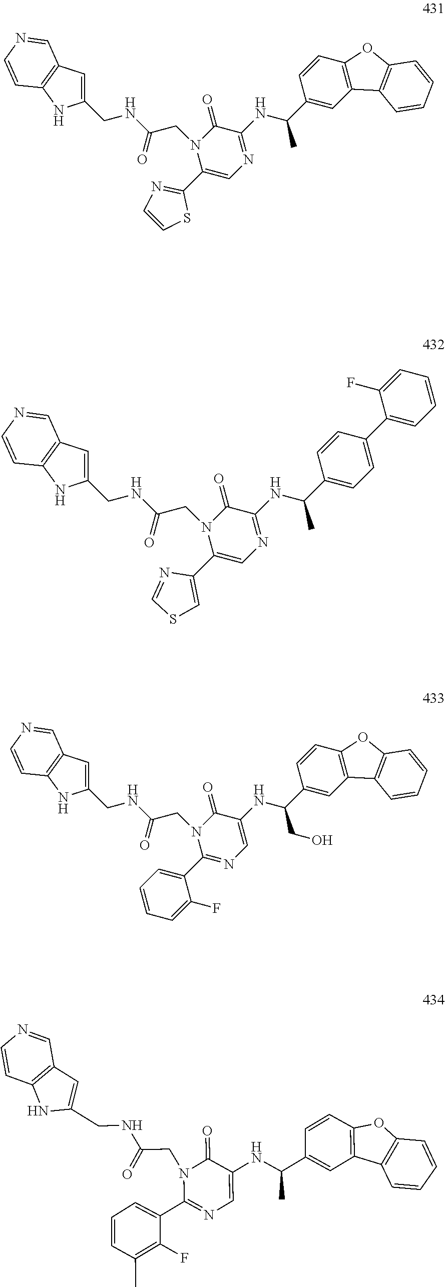 [SMILES: C[C@@H](Nc1ncc(-c2nccs2)n(CC(=O)NCc2cc3cnccc3[nH]2)c1=O)c1ccc2oc3ccccc3c2c1 ; C[C@@H](Nc1ncc(-c2cscn2)n(CC(=O)NCc2cc3cnccc3[nH]2)c1=O)c1ccc(-c2ccccc2F)cc1 ; O=C(Cn1c(-c2ccccc2F)ncc(N[C@H](CO)c2ccc3oc4ccccc4c3c2)c1=O)NCc1cc2cnccc2[nH]1 ; Cc1cccc(-c2ncc(N[C@H](C)c3ccc4oc5ccccc5c4c3)c(=O)n2CC(=O)NCc2cc3cnccc3[nH]2)c1F] 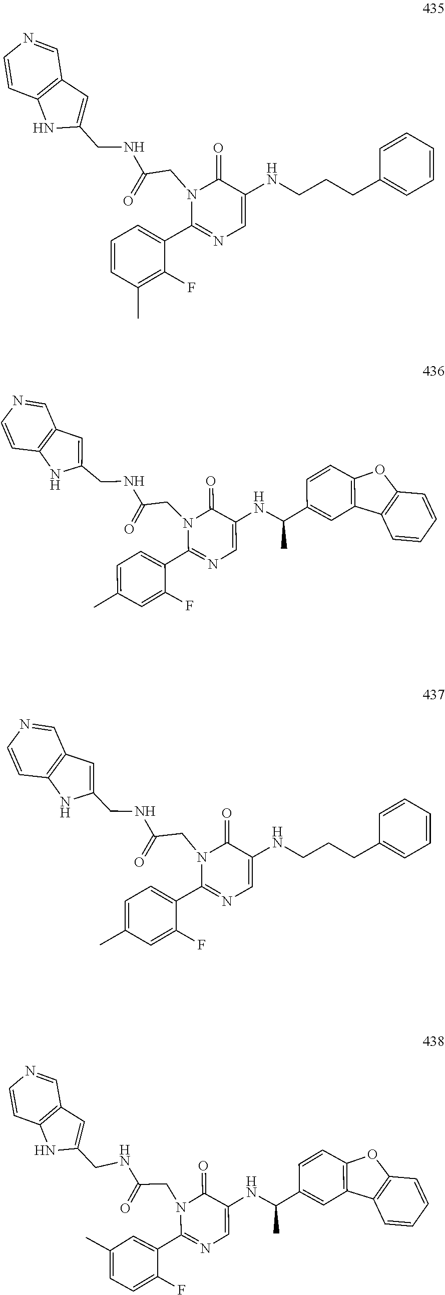 [SMILES: Cc1cccc(-c2ncc(NCCCc3ccccc3)c(=O)n2CC(=O)NCc2cc3cnccc3[nH]2)c1F ; Cc1ccc(-c2ncc(N[C@H](C)c3ccc4oc5ccccc5c4c3)c(=O)n2CC(=O)NCc2cc3cnccc3[nH]2)c(F)c1 ; Cc1ccc(-c2ncc(NCCCc3ccccc3)c(=O)n2CC(=O)NCc2cc3cnccc3[nH]2)c(F)c1 ; Cc1ccc(F)c(-c2ncc(N[C@H](C)c3ccc4oc5ccccc5c4c3)c(=O)n2CC(=O)NCc2cc3cnccc3[nH]2)c1] 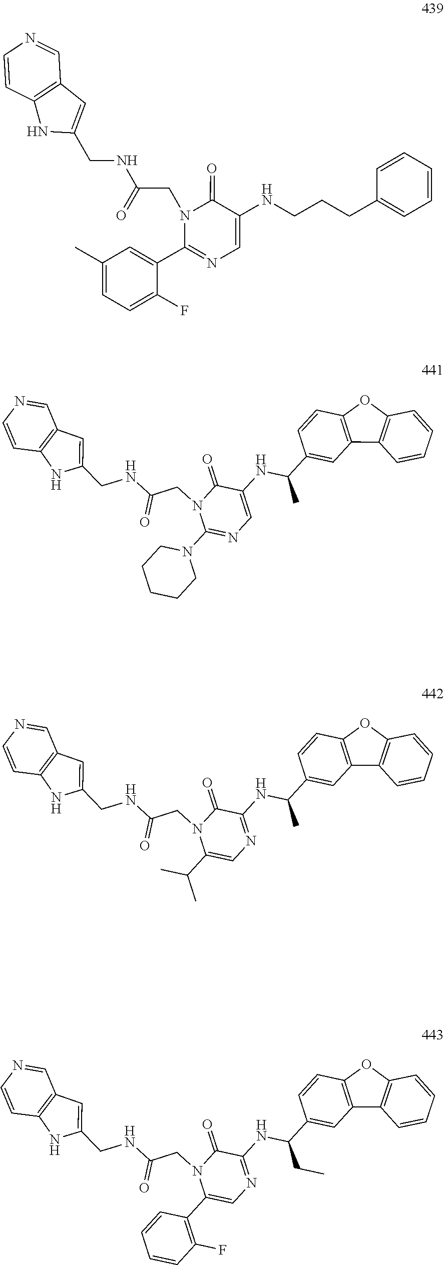 [SMILES: Cc1ccc(F)c(-c2ncc(NCCCc3ccccc3)c(=O)n2CC(=O)NCc2cc3cnccc3[nH]2)c1 ; C[C@@H](Nc1cnc(N2CCCCC2)n(CC(=O)NCc2cc3cnccc3[nH]2)c1=O)c1ccc2oc3ccccc3c2c1 ; CC(C)c1cnc(N[C@H](C)c2ccc3oc4ccccc4c3c2)c(=O)n1CC(=O)NCc1cc2cnccc2[nH]1 ; CC[C@@H](Nc1ncc(-c2ccccc2F)n(CC(=O)NCc2cc3cnccc3[nH]2)c1=O)c1ccc2oc3ccccc3c2c1] 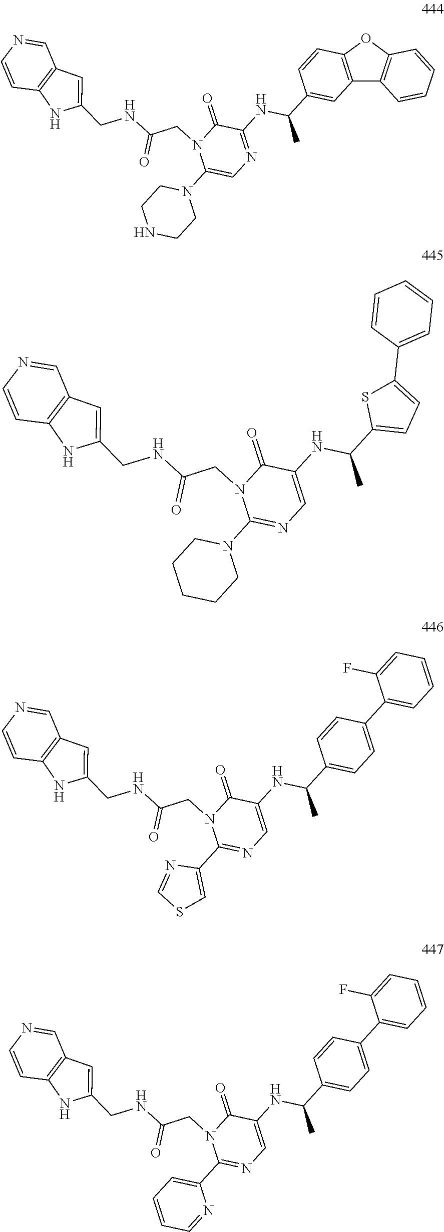 [SMILES: C[C@@H](Nc1ncc(N2CCNCC2)n(CC(=O)NCc2cc3cnccc3[nH]2)c1=O)c1ccc2oc3ccccc3c2c1 ; C[C@@H](Nc1cnc(N2CCCCC2)n(CC(=O)NCc2cc3cnccc3[nH]2)c1=O)c1ccc(-c2ccccc2)s1 ; C[C@@H](Nc1cnc(-c2cscn2)n(CC(=O)NCc2cc3cnccc3[nH]2)c1=O)c1ccc(-c2ccccc2F)cc1 ; C[C@@H](Nc1cnc(-c2ccccn2)n(CC(=O)NCc2cc3cnccc3[nH]2)c1=O)c1ccc(-c2ccccc2F)cc1] 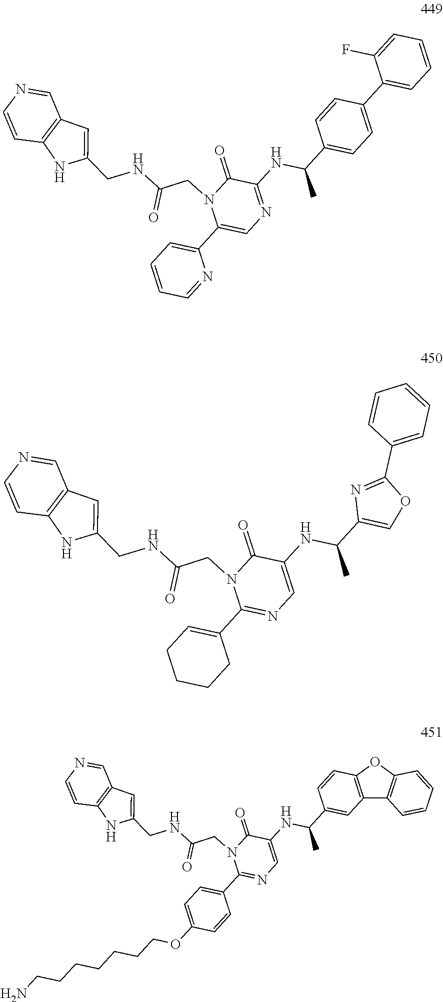 [SMILES: C[C@@H](Nc1ncc(-c2ccccn2)n(CC(=O)NCc2cc3cnccc3[nH]2)c1=O)c1ccc(-c2ccccc2F)cc1 ; C[C@@H](Nc1cnc(C2=CCCCC2)n(CC(=O)NCc2cc3cnccc3[nH]2)c1=O)c1coc(-c2ccccc2)n1 ; C[C@@H](Nc1cnc(-c2ccc(OCCCCCCCN)cc2)n(CC(=O)NCc2cc3cnccc3[nH]2)c1=O)c1ccc2oc3ccccc3c2c1] 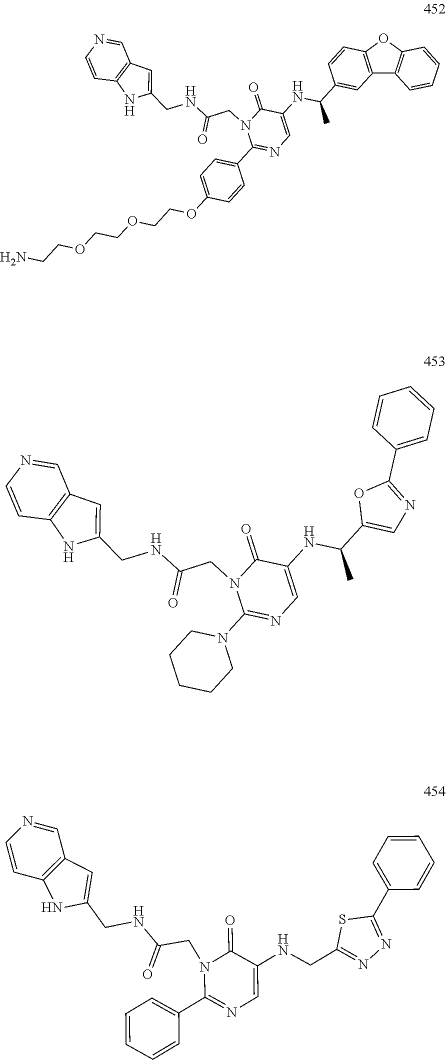 [SMILES: C[C@@H](Nc1cnc(-c2ccc(OCCOCCOCCN)cc2)n(CC(=O)NCc2cc3cnccc3[nH]2)c1=O)c1ccc2oc3ccccc3c2c1 ; C[C@@H](Nc1cnc(N2CCCCC2)n(CC(=O)NCc2cc3cnccc3[nH]2)c1=O)c1cnc(-c2ccccc2)o1 ; O=C(Cn1c(-c2ccccc2)ncc(NCc2nnc(-c3ccccc3)s2)c1=O)NCc1cc2cnccc2[nH]1] 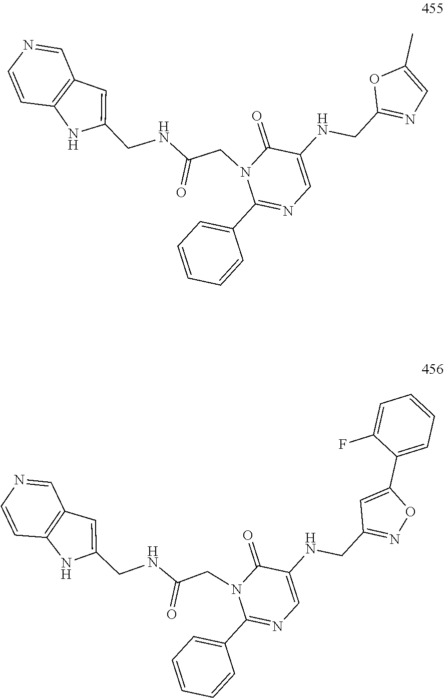 [SMILES: Cc1cnc(CNc2cnc(-c3ccccc3)n(CC(=O)NCc3cc4cnccc4[nH]3)c2=O)o1 ; O=C(Cn1c(-c2ccccc2)ncc(NCc2cc(-c3ccccc3F)on2)c1=O)NCc1cc2cnccc2[nH]1] 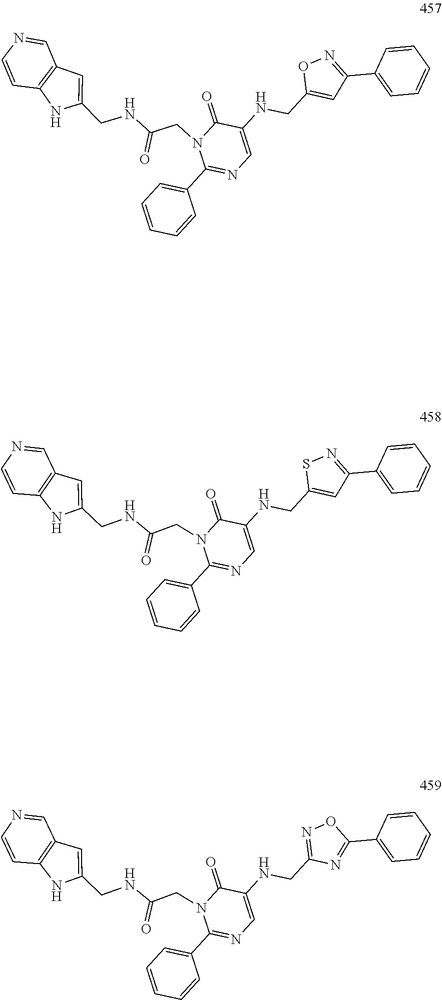 [SMILES: O=C(Cn1c(-c2ccccc2)ncc(NCc2cc(-c3ccccc3)no2)c1=O)NCc1cc2cnccc2[nH]1 ; O=C(Cn1c(-c2ccccc2)ncc(NCc2cc(-c3ccccc3)ns2)c1=O)NCc1cc2cnccc2[nH]1 ; O=C(Cn1c(-c2ccccc2)ncc(NCc2noc(-c3ccccc3)n2)c1=O)NCc1cc2cnccc2[nH]1] 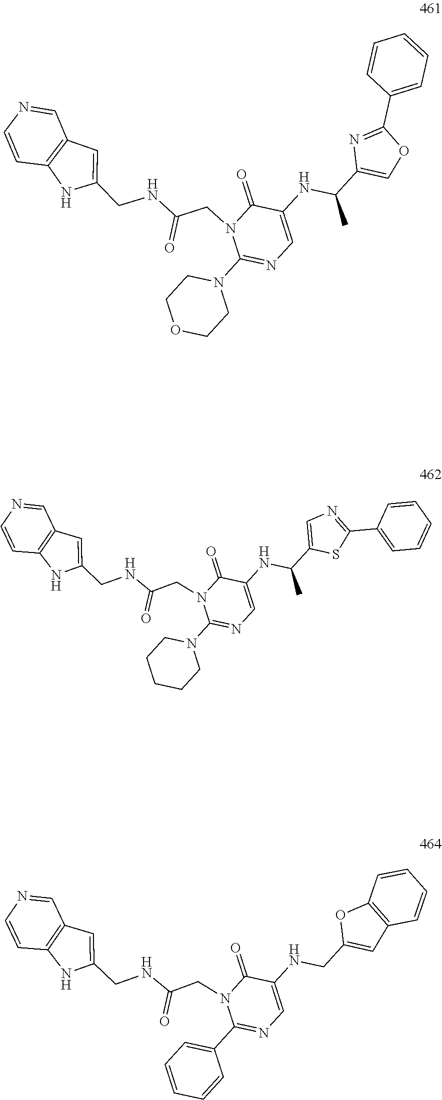 [SMILES: C[C@@H](Nc1cnc(N2CCOCC2)n(CC(=O)NCc2cc3cnccc3[nH]2)c1=O)c1coc(-c2ccccc2)n1 ; C[C@@H](Nc1cnc(N2CCCCC2)n(CC(=O)NCc2cc3cnccc3[nH]2)c1=O)c1cnc(-c2ccccc2)s1 ; O=C(Cn1c(-c2ccccc2)ncc(NCc2cc3ccccc3o2)c1=O)NCc1cc2cnccc2[nH]1] 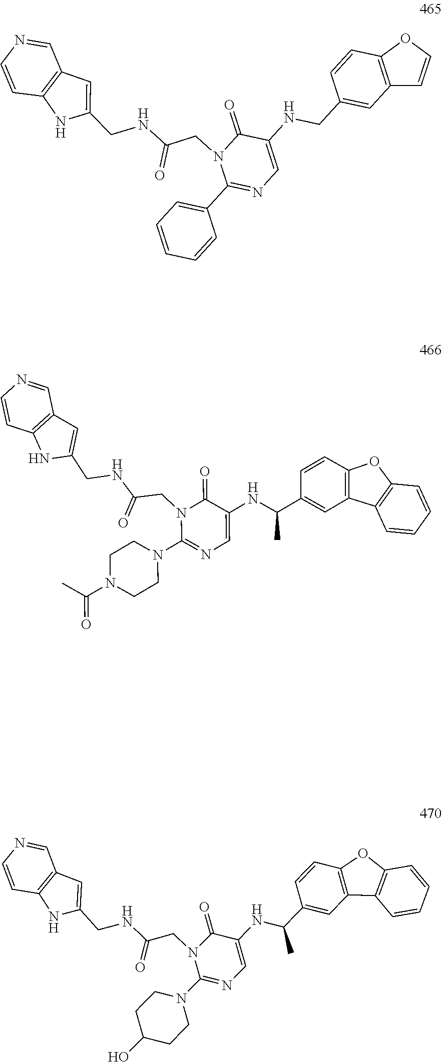 [SMILES: O=C(Cn1c(-c2ccccc2)ncc(NCc2ccc3occc3c2)c1=O)NCc1cc2cnccc2[nH]1 ; CC(=O)N1CCN(c2ncc(N[C@H](C)c3ccc4oc5ccccc5c4c3)c(=O)n2CC(=O)NCc2cc3cnccc3[nH]2)CC1 ; C[C@@H](Nc1cnc(N2CCC(O)CC2)n(CC(=O)NCc2cc3cnccc3[nH]2)c1=O)c1ccc2oc3ccccc3c2c1] 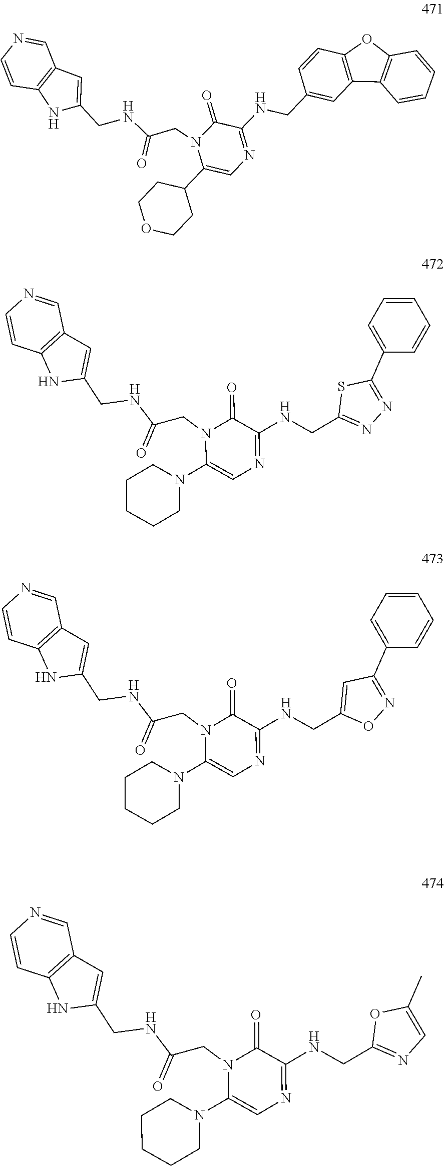 [SMILES: O=C(Cn1c(C2CCOCC2)cnc(NCc2ccc3oc4ccccc4c3c2)c1=O)NCc1cc2cnccc2[nH]1 ; O=C(Cn1c(N2CCCCC2)cnc(NCc2nnc(-c3ccccc3)s2)c1=O)NCc1cc2cnccc2[nH]1 ; O=C(Cn1c(N2CCCCC2)cnc(NCc2cc(-c3ccccc3)no2)c1=O)NCc1cc2cnccc2[nH]1 ; Cc1cnc(CNc2ncc(N3CCCCC3)n(CC(=O)NCc3cc4cnccc4[nH]3)c2=O)o1] 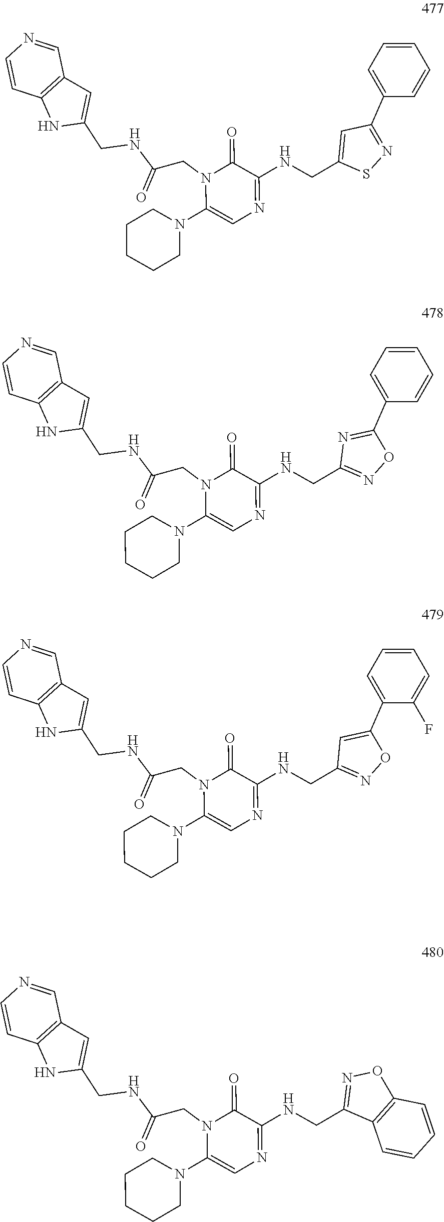 [SMILES: O=C(Cn1c(N2CCCCC2)cnc(NCc2cc(-c3ccccc3)ns2)c1=O)NCc1cc2cnccc2[nH]1 ; O=C(Cn1c(N2CCCCC2)cnc(NCc2noc(-c3ccccc3)n2)c1=O)NCc1cc2cnccc2[nH]1 ; O=C(Cn1c(N2CCCCC2)cnc(NCc2cc(-c3ccccc3F)on2)c1=O)NCc1cc2cnccc2[nH]1 ; O=C(Cn1c(N2CCCCC2)cnc(NCc2noc3ccccc23)c1=O)NCc1cc2cnccc2[nH]1] 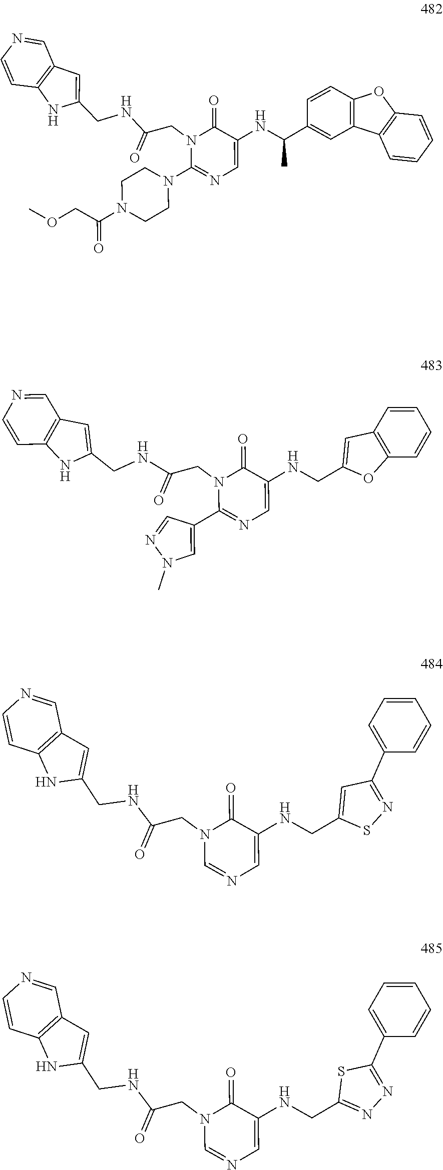 [SMILES: COCC(=O)N1CCN(c2ncc(N[C@H](C)c3ccc4oc5ccccc5c4c3)c(=O)n2CC(=O)NCc2cc3cnccc3[nH]2)CC1 ; Cn1cc(-c2ncc(NCc3cc4ccccc4o3)c(=O)n2CC(=O)NCc2cc3cnccc3[nH]2)cn1 ; O=C(Cn1cncc(NCc2cc(-c3ccccc3)ns2)c1=O)NCc1cc2cnccc2[nH]1 ; O=C(Cn1cncc(NCc2nnc(-c3ccccc3)s2)c1=O)NCc1cc2cnccc2[nH]1] 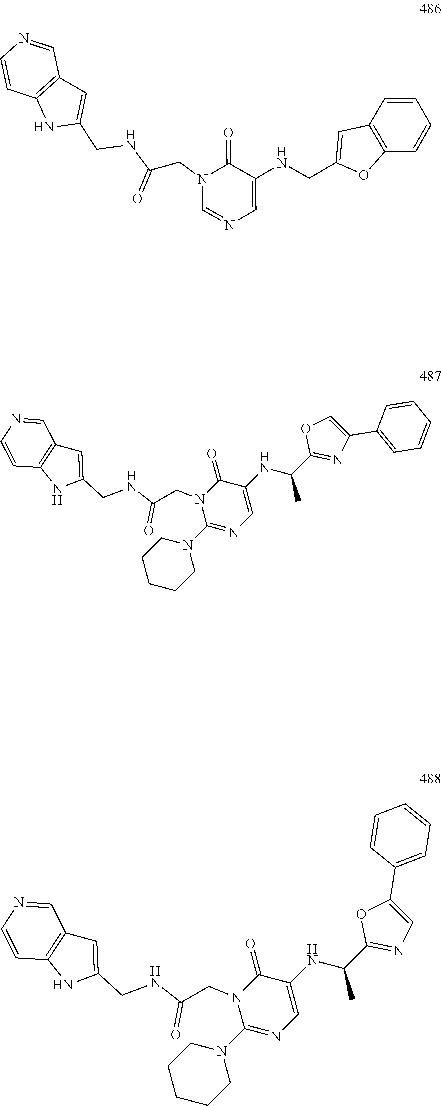 [SMILES: O=C(Cn1cncc(NCc2cc3ccccc3o2)c1=O)NCc1cc2cnccc2[nH]1 ; C[C@@H](Nc1cnc(N2CCCCC2)n(CC(=O)NCc2cc3cnccc3[nH]2)c1=O)c1nc(-c2ccccc2)co1 ; C[C@@H](Nc1cnc(N2CCCCC2)n(CC(=O)NCc2cc3cnccc3[nH]2)c1=O)c1ncc(-c2ccccc2)o1] 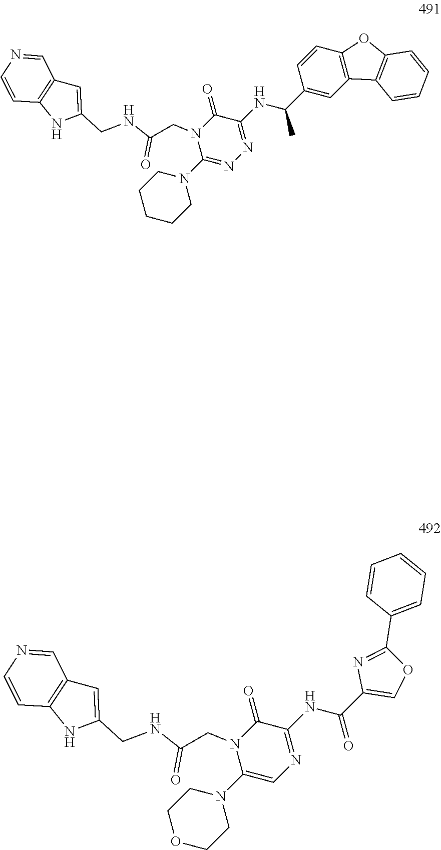 [SMILES: C[C@@H](Nc1nnc(N2CCCCC2)n(CC(=O)NCc2cc3cnccc3[nH]2)c1=O)c1ccc2oc3ccccc3c2c1 ; O=C(Cn1c(N2CCOCC2)cnc(NC(=O)c2coc(-c3ccccc3)n2)c1=O)NCc1cc2cnccc2[nH]1]
or a pharmaceutically acceptable salt thereof.

95. (canceled)

96. A pharmaceutical composition, comprising a compound of claim 1 or a pharmaceutically acceptable salt thereof, and a pharmaceutically acceptable excipient.

97. A method of treating a C1s mediated disorder, comprising administering to a human subject in need thereof a therapeutically effective amount of a compound of Formula (I′): 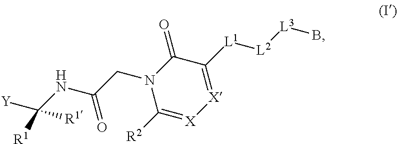
or a pharmaceutically acceptable salt thereof,
wherein
each of R¹ and R^{1′} is independently H or optionally substituted C₁-C₆ alkyl;
X is CR³ or N;
X is CR⁴ or N;
R² is H, optionally substituted C₁-C₆ alkyl, optionally substituted C₆-C₁₄ aryl, optionally substituted 5- to 10-membered heteroaryl, optionally substituted C₃-C₈ carbocyclyl, optionally substituted 4- to 10-membered heterocyclyl, or optionally substituted (4- to 10-membered heterocyclyl)oxy, or optionally substituted (5- to 9-membered heteroaryl)oxy;
each R³ and R⁴ is independently H, halo, or optionally substituted C₁-C₆ alkyl;
L¹ is a bond; NH; NHC(O); NHC(O)O; NHC(O)NH, or NHS(O)₂;
L² is a bond or optionally substituted C₁-C₆ alkylene;
L³ is a bond, NH, NHC(O), C(O), O, S(O)₂CH₂;
B is halo, optionally substituted C₆-C₁₄ aryl; optionally substituted C₃-C₁₄ carbocyclyl; optionally substituted 5- to 14-membered heterocyclyl; or optionally substituted 5- to 10-membered heteroaryl; and
Y is 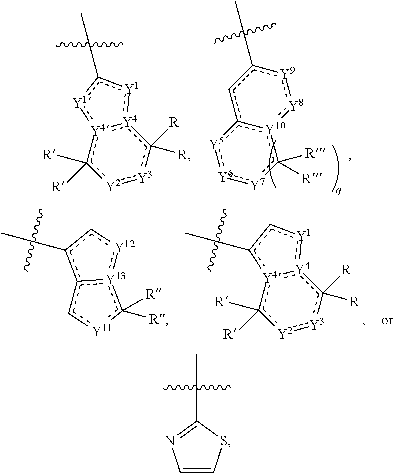
wherein
Y¹ is O, S, NR^{d}, wherein each R^{d} is independently absent, H, or C₁-C₆ alkyl;
Y^{1′} is O, S, NR^{d}, or C(R^{d})₂;
each of Y² and Y³ is independently NR^{e} or C(R^{e})₂, wherein each R^{e} is independently absent; H; optionally substituted C₁-C₆ alkyl; halo; or N(R^{g})₂, wherein each R^{g} is independently H or C₁-C₆ alkyl; or both R^{e} combine to form oxo;
each of Y⁴, Y^{4′}, Y¹⁰, and Y¹³ is independently CR^{e} or N;
each of Y⁵, Y⁶, and Y⁷ is independently O, S, NR^{f} or C(R^{f})₂, wherein each R^{f} is independently absent; H; optionally substituted C₁-C₆ alkyl; halo; or N(R^{g})₂; or both R^{f} combine to form oxo;
each of Y⁸ and Y⁹ is independently C(R^{f})₂ or NR^{f};
each of Y¹¹ and Y¹² is independently NR^{e}, C(R^{e})₂, S, or O;
each [symbol] is independently a single bond or a double bond;
each of R, R′, R″, and R′″ is independently absent, H, optionally substituted C₁-C₆ alkyl, halo, or N(R^{g})₂; or
both R combine to form oxo; or
both R′ combine to form oxo; and
q is 0 or 1;
wherein the disorder is acute antibody-mediated rejection, amyotrophic lateral sclerosis, autoimmune blistering disease, bullous pemphigoid, chronic inflammatory demyelinating polyneuropathy, geographic atrophy, Guillain-Barre Syndrome, Huntington's Disease, immune thrombocytopenia purpura, lupus nephritis, multifocal motor neuropathy, rheumatoid arthritis, traumatic brain injury, and warm autoimmune hemolytic anemia.

98-100. (canceled)
